(19) United States
(12) Patent Application Publication
(10) Pub. No.: US 2023/0123669 A1
(43) Pub. Date: Apr. 20, 2023

(54) BASE EDITOR PREDICTIVE ALGORITHM AND METHOD OF USE

(71) Applicants: The Broad Institute, Inc., Cambridge, MA (US); President and Fellows of Harvard College, Cambridge, MA (US); Massachusetts Institute of Technology, Cambridge, MA (US); The Brigham and Women's Hospital, Inc., Boston, MA (US)

(72) Inventors: David R. Liu, Cambridge, MA (US); Mandana Arbab, Cambridge, MA (US); Max Walt Shen, Cambridge, MA (US); Christopher Cassa, Boston, MA (US)

(73) Assignees: The Broad Institute, Inc., Cambridge, MA (US); President and Fellows of Harvard College, Cambridge, MA (US); Massachusetts Institute of Technology, Cambridge, MA (US); The Brigham and Woman's Hospital, Inc., Boston, MA (US)

(21) Appl. No.: 17/797,697

(22) PCT Filed: Feb. 5, 2021
(86) PCT No.: PCT/US2021/016924

§ 371 Date: Aug. 4, 2022

Related U.S. Application Data
(60) Provisional application No. 63/038,691, filed on Jun. 12, 2020.
(60) Provisional application No. 62/970,684, filed on Feb. 5, 2020.

(51) Int. Cl. G16B 40/00; G16B 20/50; C12N 15/11

(52) U.S. Cl. CPC G16B 40/00; G16B 20/50; C12N 15/11

(57) ABSTRACT

The present disclosure provides a novel machine learning model capable of assisting those of ordinary skill in the art to conduct base editing by, inter alia, facilitating the selection of an appropriate guide RNA and base editor combination which are capable of conducting base editing at a certain level of efficiency and specificity on a given input target DNA sequence desired to be edited to produce an outcome genotype of interest. The disclosure also provides base editors (e.g., ABEs and CBEs), napDNAbps, cytidine deaminases, adenosine deaminases, nucleic acid sequences encoding base editors and components thereof, vectors, and cells. In addition, the disclosure provides methods of making biological or experimental training and/or validation data for training and/or validating the machine learning computational models, as well as, vectors, libraries, and nucleic acid sequences for use in obtaining said experimental training and/or validation data.

### RELATED APPLICATIONS

This PCT application claims the benefit under 35 U.S.C. § 119(e) of U.S. Provisional Application No. 62/970,684, filed Feb. 5, 2020, and to U.S. Provisional Application No. 63/038,691, filed Jun. 12, 2020. The entire contents of each of the above-indicated applications are incorporated herein by reference in their entireties.

### GOVERNMENT SUPPORT

This invention was made with government support under AI142756, HG009490, EB022376, GM118062, HG010372, and HG010391 awarded by the National Institutes of Health; and HR0011-17-2-0049, awarded by the Defense Advanced Research Projects Agency. The government has certain rights in the invention.

### BACKGROUND OF THE INVENTION

Programmable editing of single nucleotides in genomic DNA is a key capability for both research and therapeutic applications (Adli, 2018; Anzalone et al., 2019; Doench et al., 2016; Doudna and Knott, 2018; Pérez-Palma et al., 2019; Rees and Liu, 2018; Shen et al., 2018). Single-nucleotide variants (SNVs) represent approximately half of known pathogenic alleles (Landrum et al., 2016; Stenson et al., 2014), and thus targeted installation of point mutations can facilitate the study or potential treatment of genetic disorders. Previously, cytosine deaminases were developed, and laboratory-evolved adenine deaminase enzymes fused to catalytically impaired CRISPR-Cas proteins to enable cytosine and adenine base editing in living cells in a programmable fashion without requiring a DNA double-strand break or a donor DNA template (Gaudelli et al., 2017; Gehrke et al., 2018; Huang et al., 2019; Komor et al., 2016; Nishida et al., 2016; Thuronyi et al., 2019; Yeh et al., 2018). Cytosine base editors (CBEs) and adenine base editors (ABEs) together enable all four transition point mutations (C→T, T→C, A→G, and G→A) and routinely achieve high ratios of desired sequence substitutions relative to undesired insertions and deletions (indels) (Lin et al., 2014; Paquet et al., 2016). Base editing has been applied in a wide range of organisms ranging from bacteria to plants to primates (Rees and Liu, 2018), and has already been used to correct pathogenic mutations in animal models, in some cases with phenotypic rescue (Chadwick et al., 2017; Liang et al., 2017; Min et al., 2019; Ryu et al., 2018; Song et al., 2019; Villiger et al., 2018; Yeh et al., 2018; Zeng et al., 2018), establishing its potential for clinical applications.

The utility of base editing has inspired the development of many cytosine and adenine base editor variants with distinct editing properties (Adli, 2018; Molla and Yang, 2019; Rees and Liu, 2018). To date, these properties have been gleaned by analyzing base editing outcomes at a modest number of genomic sites, often chosen to align with previous genome editing studies (Gaudelli et al., 2017; Gehrke et al., 2018; Huang et al., 2019; Komor et al., 2016; Thuronyi et al., 2019). The interplay between base editor and target sequence, however, influences base editing outcomes in complex and occasionally unintuitive ways (Gehrke et al., 2018; Huang et al., 2019; Tan et al., 2019; Thuronyi et al., 2019; Villiger et al., 2018). As a result, obtaining a desired genotype with useful efficiencies often requires empirical optimization of base editor and single guide RNA (sgRNA) choice for each target. Likewise, some viable targets that do not fit canonical guidelines for base editing use may be overlooked since simple guidelines for target selection likely do not fully capture the scope of base editing.

A predictive tool that facilitates the selection of appropriate base editors and/or guide RNAs to achieve any given desired genotype outcome for a given target site through base editing would be a significant advancement in the art.

### SUMMARY OF THE INVENTION

The inventors have determined that base editing outcomes are highly dependent on both the particular base editor and the target sequence context and cannot be reliably predicted from the target locus and known base editor characteristics by simple inspection. The abundance of base editors designed for the same basic task complicates selection of the optimal tool for precision editing at a locus of interest. Through a comprehensive and systematic analysis of sequence and base editor determinants of base editing outcomes as described herein (e.g., in the Examples), the inventors have built of a suite of machine learning models for predicting genome outcomes in base editing, and for facilitating the selection of appropriate base conditions (e.g., the particular base editor employed and guide RNA used) for any given genomic locus and desired genotype outcome.

Accordingly, the present disclosure provides novel machine learning models capable of assisting those of ordinary skill in the art to conduct base editing by, inter alia, facilitating the selection of an appropriate guide RNA and base editor combination which are capable of conducting base editing at a certain level of efficiency and specificity on a given input target DNA sequence desired to be edited to produce an outcome genotype of interest. The novel machine learning algorithm described and claimed herein can be referred to as “BE-Hive.” The disclosure further provides a graphical user interface that implements BE-Hive, allowing a user to input various features, including a desired target DNA sequence, an appropriate guide RNA (or associated CRISPR protospacer), a base editor, and a cell in which base editing is to take place, and to predict base editing efficiencies and bystander editing patterns for the selected features.

The disclosure provides systematic and comprehensive predictive tools (e.g., one or more machine learning models) that facilitate the selection of appropriate base editors and/or guide RNAs to achieve any given desired predicted genotype outcome for a given target site through base editing. In another aspect, the predictive tools (e.g., machine learning models) disclosed herein may also be used to discover or identify previously unknown base editor properties (e.g., previously unknown preferences, such as a base editor's preference to make a transversion edit instead of a transition edit), which may facilitate the design of novel base editors with new capabilities. In various aspects, the herein disclosed machine learning models for selecting base editing components (e.g., selecting an appropriate base editor and/or a guide RNA) to achieve a desired genotype outcome may involve the consideration of one or more determinants of base editing, which can include, but are not limited to, the choice of the napDNAbp of the base editing system; the choice of the deaminase of the base editing system; the choice of base editor; the target nucleotide sequence (e.g., guide RNA binding sites); the target genomic location; the transcriptional state of the target genomic location; locus-dependent activity of the choice napDNAbp; cell-type; transcriptional state of DNA repair proteins; and base editor modifications.

The disclosure also provides machine learning models for predicting genotype outcomes based on one or more inputs, such as a base editor and/or other determinants of base editing, which include, but are not limited to, the choice of the napDNAbp of the base editing system; the choice of the deaminase of the base editing system; the choice of base editor; the target nucleotide sequence (e.g., guide RNA binding sites); the target genomic location; the transcriptional state of the target genomic location; locus-dependent activity of the choice napDNAbp; cell-type; transcriptional state of DNA repair proteins; and base editor modifications.

In addition, the disclosure provides methods of training the machine learning models used herein to be able to predict desired genotype outcomes based on one or more inputs, such as a base editor and/or other determinants of base editing, which include, but are not limited to, the choice of the napDNAbp of the base editing system; the choice of the deaminase of the base editing system; the choice of base editor; the target nucleotide sequence (e.g., guide RNA binding sites); the target genomic location; the transcriptional state of the target genomic location; locus-dependent activity of the choice napDNAbp; cell-type; transcriptional state of DNA repair proteins; and base editor modifications.

In certain other aspects, the disclosure provides training methods for the herein disclosed machine learning models. In certain aspects, the training methods comprises obtaining training data for training the machine learning models. The training data, in some aspects, may comprising sequencing information generated from a plurality of base editing reactions conducted in cells comprising a base editor, a guide RNA, and an editing target, wherein sequencing the DNA in the edited cells produces sequencing data that may be analyzed to identify the nucleotide edits made for a particular base editor.

The disclosure further provides base editors (e.g., ABEs and CBEs), napDNAbps, cytidine deaminases, adenosine deaminases, guide RNAs, nucleic acid sequences encoding base editors and components thereof, nucleic acid sequences encoding guide RNAs, vectors that encode base editors and/or guide RNAs and/or target sites of interest, training libraries comprising a plurality of vectors for generating sequencing data of actual genotype outcomes of base editing reactions for use in training the computation models described herein, and cells comprising said vectors and training libraries, all of which may be used in connection with the machine learning models described herein to predict desired genotype outcomes of a target site of interest.

In one aspect, the disclosure provides a method of using at least one machine learning model to identify a guide RNA for use in a base editing system for introducing a target change into a nucleotide sequence, the base editing system comprising a napDNAbp and a deaminase, the method comprising: using software executing on at least one computer hardware processor to perform: obtaining input data indicative of the nucleotide sequence and a set of one or more guide RNAs; generating first input features from the input data; applying a first machine learning model to the first input features to obtain first output data indicative, for each one guide RNA in the set of guide RNAs, of a base editing efficiency, at one or multiple locations in the nucleotide sequence, of the base editing system when using the each one guide RNA; generating second input features from the input data; applying a second machine learning model to the second input features to obtain second output data indicative, for each one guide RNA in the set of guide RNAs, of bystander editing activity, at one or multiple locations in the nucleotide sequence, by the base editing system when using the each one guide RNA; and identifying, using the first output data and the second output data, the guide RNA for use in the base editing system for introducing the target change into the nucleotide sequence.

In certain embodiments, the first machine learning model comprises a non-linear machine learning model selected from the group consisting of a random forest model, a logistic regression model, a support vector machine model, a generalized linear model, a hierarchical Bayesian model, and neural network model. In other embodiments, the first machine learning model can comprise a random forest model.

The set of guide RNAs can include a first guide RNA, and wherein generating the first input features comprises generating multiple features to include in the first input features, the multiple features including: features encoding at least some nucleotides in a protospacer sequence or spacer sequence associated with the first guide RNA; and features encoding at least some nucleotides, in the nucleotide sequence, located within a threshold number of nucleotides of the protospacer sequence associated with the first guide RNA.

In various embodiments, the multiple features further include one or more of the following features: features encoding at least some dinucleotides at neighboring positions in the protospacer sequence; features representing melting temperature of the first guide RNA; one or more features representing a total number of G, C, A, and/or T nucleotides in the protospacer sequence; and a feature representing an average base editing efficiency of the base editing system.

In certain embodiments, the set of guide RNAs includes a first guide RNA, wherein the first output data is indicative of a fraction of sequence reads containing at least one base edit at any nucleotide in a target window about a protospacer sequence associated with the first guide RNA, among all sequence reads.

In other embodiments, the second first machine learning model comprises a non-linear machine learning model selected from the group consisting of a random forest model, a logistic regression model, a support vector machine model, a generalized linear model, a hierarchical Bayesian model, and neural network model. In yet other embodiments, the second machine learning model comprises a deep neural network model. The neural network model can comprise a conditional autoregressive neural network model. The conditional autoregressive neural network model can include: an encoder neural network mapping input data to a latent representation; and a decoder neural network mapping the latent representation to output data, wherein the decoder neural network has an autoregressive structure. The encoder neural network can comprise a multi-layer fully connected network with residual connections. The decoder neural network can generate a distribution over base editing outcomes at each nucleotide while conditioning on previously-generated outcomes. The neural network model can include parameters representing a position-wise bias toward producing an unedited outcome.

The set of guide RNAs can include a first guide RNA, and wherein generating the second input features can comprise generating multiple features to include in the second input features, the multiple features including: features encoding at least some nucleotides in a protospacer sequence or spacer sequence associated with the first guide RNA; and features encoding at least some nucleotides, in the nucleotide sequence, located within a threshold number of nucleotides of the protospacer sequence associated with the first guide RNA.

In other embodiments, the second output data can be indicative of frequencies of occurrence of base editing outcomes each of which includes edits to nucleotides at multiple positions. The second output data can be indicative of a frequency distribution on combinations of base editing outcomes.

In various embodiments, the set of guide RNAs can include a first guide RNA, wherein, for a specific combination of base edits, the second output data is indicative of a frequency of occurrence of the specific combination of base edits among all sequenced reads containing at least one base edit at any nucleotide in a target window about a protospacer sequence associated with the first guide RNA.

In other embodiments, the set of guide RNAs can include a first guide RNA, wherein the first output data includes a first base editing efficiency value for the first guide RNA, wherein the second output data includes a first bystander editing value for the first guide RNA, and wherein identifying the guide RNA using the first output data and the second output data, comprises multiplying the first base editing efficiency value by the first bystander editing value.

In certain embodiments, the first machine learning model comprises a first plurality of values for a respective first plurality of parameters, the first plurality of values used by the at least one computer hardware processor to obtain the first output data from the first input features. The first plurality of parameters can comprise at least one thousand parameters. The first plurality of parameters can comprise between one thousand and ten thousand parameters.

In various embodiments, the first machine learning model can comprise a random forest model comprising at least 100 decision trees, each of the at least 100 decision trees having at least a depth of D, and wherein processing the input data using the random forest model comprises performing 100*D comparisons. The random forest model can comprise at least 500 decision trees. In certain embodiments, depth of D can be greater than or equal to five, wherein processing the input data using the random forest model comprises performing at least 2500 comparisons.

In other embodiments, the second machine learning model can comprise a second plurality of values for a respective second plurality of parameters, the second plurality of values used by the at least one computer hardware processor to obtain the second output data from the second input features. The second plurality of parameters can comprise at least ten thousand parameters, or between 25,000 and 100,000 parameters, or between 30,000 and 40,000 parameters.

In other embodiments, the disclosure provides a method of manufacturing the identified guide RNA for use in the base editing system for introducing the target change into the nucleotide sequence.

In other aspects, the disclosure provides a method for training the first machine learning model of any of the above aspects comprising: (i) preparing a library comprising a plurality of nucleic acid molecules each encoding a nucleotide target sequence and a cognate guide RNA; (ii) introducing the library into a plurality of host cells; (iii) contacting the library in the host cells with a Cas-based genome editing system to produce a plurality of genomic repair products; (iv) determining the sequences of the genomic repair products; and (v) training the first machine learning model with training data that comprises at least the sequences of the genomic repair products and the cognate guide RNA.

In still other embodiments, the disclosure provides a method for training the second machine learning model of any of the above aspects comprising: (i) preparing a library comprising a plurality of nucleic acid molecules each encoding a nucleotide target sequence and a cognate guide RNA; (ii) introducing the library into a plurality of host cells; (iii) contacting the library in the host cells with a Cas-based genome editing system to produce a plurality of genomic repair products; (iv) determining the sequences of the genomic repair products; and (v) training the second machine learning model with training data that comprises at least the sequences of the genomic repair products and the cognate guide RNA.

The disclosure also provides for a computer readable storage medium storing processor executable instructions that, when executed by at least one computer hardware processor, cause the at least one computer hardware processor to perform a method of using at least one machine learning model to identify a guide RNA for use in a base editing system for introducing a target change into a nucleotide sequence, the base editing system comprising a napDNAbp and a deaminase, the method comprising: obtaining input data indicative of the nucleotide sequence and a set of one or more guide RNAs; generating first input features from the input data; applying a first machine learning model to the first input features to obtain first output data indicative, for each one guide RNA in the set of guide RNAs, of a base editing efficiency, at one or multiple locations in the nucleotide sequence, of the base editing system when using the each one guide RNA; generating second input features from the input data; applying a second machine learning model to the second input features to obtain second output data indicative, for each one guide RNA in the set of guide RNAs, of bystander editing activity, at one or multiple locations in the nucleotide sequence, by the base editing system when using the each one guide RNA; and identifying, using the first output data and the second output data, the guide RNA for use in the base editing system for introducing the target change into the nucleotide sequence.

In another aspect, the disclosure provides a system comprising: at least one computer hardware processor; and at least one computer readable storage medium storing processor executable instructions that, when executed by the at least one computer hardware processor, cause the at least one computer hardware processor to perform a method of using at least one machine learning model to identify a guide RNA for use in a base editing system for introducing a target change into a nucleotide sequence, the base editing system comprising a napDNAbp and a deaminase, the method comprising: obtaining input data indicative of the nucleotide sequence and a set of one or more guide RNAs; generating first input features from the input data; applying a first machine learning model to the first input features to obtain first output data indicative, for each one guide RNA in the set of guide RNAs, of a base editing efficiency, at one or multiple locations in the nucleotide sequence, of the base editing system when using the each one guide RNA; generating second input features from the input data; applying a second machine learning model to the second input features to obtain second output data indicative, for each one guide RNA in the set of guide RNAs, of bystander editing activity, at one or multiple locations in the nucleotide sequence, by the base editing system when using the each one guide RNA; and identifying, using the first output data and the second output data, the guide RNA for use in the base editing system for introducing the target change into the nucleotide sequence.

In other aspects, the disclosure provides a method, comprising: using software executing on at least one computer hardware processor to perform: receiving input data indicative of a selection of: a nucleotide sequence; a base editing system comprising a napDNAbp and a deaminase; and a first guide RNA; applying a first machine learning model to the first input features, generated from the input data, to obtain first output data indicative of a base editing efficiency, at a target location in the nucleotide sequence, of the base editing system when using the first guide RNA; applying a second machine learning model to the second input features, generated from the input data, to obtain second output data indicative of bystander editing activity, at one or multiple locations in the nucleotide sequence, by the base editing system when using the first guide RNA; and determining, using the first output data and the second output data, a likelihood of whether the first guide RNA and the base editing system, when used in combination, will result in introduce a target change to the nucleotide sequence in a cell.

The disclosure also provides at least one computer-readable storage medium storing processor-executable instructions that, when executed by at least one computer hardware processor, cause the at least one computer processor to perform: receiving input data indicative of a selection of: a nucleotide sequence; a base editing system comprising a napDNAbp and a deaminase; and a first guide RNA; applying a first machine learning model to the first input features, generated from the input data, to obtain first output data indicative of a base editing efficiency, at a target location in the nucleotide sequence, of the base editing system when using the first guide RNA; applying a second machine learning model to the second input features, generated from the input data, to obtain second output data indicative of bystander editing activity, at one or multiple locations in the nucleotide sequence, by the base editing system when using the first guide RNA; and determining, using the first output data and the second output data, a likelihood of whether the first guide RNA and the base editing system, when used in combination, will result in introduce a target change to the nucleotide sequence in a cell.

In other aspects, the disclosure provides a system, comprising: at least one computer hardware processor; and at least one computer-readable storage medium storing processor-executable instructions that, when executed by the at least one computer hardware processor, cause the at least one computer processor to perform: receiving input data indicative of a selection of: a nucleotide sequence; a base editing system comprising a napDNAbp and a deaminase; and a first guide RNA; applying a first machine learning model to the first input features, generated from the input data, to obtain first output data indicative of a base editing efficiency, at a target location in the nucleotide sequence, of the base editing system when using the first guide RNA; applying a second machine learning model to the second input features, generated from the input data, to obtain second output data indicative of bystander editing activity, at one or multiple locations in the nucleotide sequence, by the base editing system when using the first guide RNA; and determining, using the first output data and the second output data, a likelihood of whether the first guide RNA and the base editing system, when used in combination, will result in introduce a target change to the nucleotide sequence in a cell.

In one aspect, the present disclosure provides a machine learning algorithm capable of assisting those of ordinary skill in the art to conduct base editing by, inter alia, facilitating the selection of an appropriate guide RNA and base editor combination which are capable of conducting base editing at a certain level of efficiency and specificity on a given input target DNA sequence desired to be edited to produce an outcome genotype of interest. The machine learning algorithm considers various inputs, including the sequence of the target DNA sequence to be edited, the napDNAbp options, the deaminase options, the guide RNA options, the spacer and/or protospacer sequence associated with the RNA options, dinucleotide composition at neighboring positions in the protospacers, guide RNA melting temperatures, and the total number of G, C, A, and/or T nucleotides in the protospacer sequence, among other features. In addition, other features that may be considered as input to the machine learning algorithm. Such features may include, but are not limited to, the transcriptional state of the target genomic location, cell-type in which the base editing is taking place, transcriptional state of the target DNA being edited, and any epigenetic modifications of the target DNA being edited.

In other aspects, the machine learning model can include or be based solely on a base editing efficiency machine learning model, for example, a method identifying a guide RNA for use in a base editing system for introducing a target change into a nucleotide sequence, the base editing system comprising a napDNAbp and a deaminase, the method comprising: using software executing on at least one computer hardware processor to perform: obtaining input data indicative of the nucleotide sequence and a set of one or more guide RNAs; generating first input features from the input data; applying a first machine learning model to the first input features to obtain first output data indicative, for each one guide RNA in the set of guide RNAs, of a base editing efficiency, at one or multiple locations in the nucleotide sequence, of the base editing system when using the each one guide RNA; and identifying, using the first output data, the guide RNA for use in the base editing system for introducing the target change into the nucleotide sequence.

Nevertheless, in such aspects, the machine learning model can further comprise a bystander model, comprising generating second input features from the input data; applying a second machine learning model to the second input features to obtain second output data indicative, for each one guide RNA in the set of guide RNAs, of bystander editing activity, at one or multiple locations in the nucleotide sequence, by the base editing system when using the each one guide RNA, wherein identifying the guide RNA is performed using the first output data and the second output data.

The disclosure also provides at least one computer readable storage medium storing processor executable instructions that, when executed by at least one computer hardware processor, cause the at least one computer hardware processor to perform a method of identifying a guide RNA for use in a base editing system for introducing a target change into a nucleotide sequence, the base editing system comprising a napDNAbp and a deaminase, the method comprising: obtaining input data indicative of the nucleotide sequence and a set of one or more guide RNAs; generating first input features from the input data; applying a first machine learning model to the first input features to obtain first output data indicative, for each one guide RNA in the set of guide RNAs, of a base editing efficiency, at one or multiple locations in the nucleotide sequence, of the base editing system when using the each one guide RNA; and identifying, using the first output data, the guide RNA for use in the base editing system for introducing the target change into the nucleotide sequence.

In other aspects, the disclosure provides a system, comprising: at least one computer hardware processor; and at least one computer readable storage medium storing processor executable instructions that, when executed by the at least one computer hardware processor, cause the at least one computer hardware processor to perform a method of identifying a guide RNA for use in a base editing system for introducing a target change into a nucleotide sequence, the base editing system comprising a napDNAbp and a deaminase, the method comprising: obtaining input data indicative of the nucleotide sequence and a set of one or more guide RNAs; generating first input features from the input data; applying a first machine learning model to the first input features to obtain first output data indicative, for each one guide RNA in the set of guide RNAs, of a base editing efficiency, at one or multiple locations in the nucleotide sequence, of the base editing system when using the each one guide RNA; and identifying, using the first output data, the guide RNA for use in the base editing system for introducing the target change into the nucleotide sequence.

Thus, in various aspects, the machine learning model can include or be based solely on a bystander machine learning model, comprising a method of identifying a guide RNA for use in a base editing system for introducing a target change into a nucleotide sequence, the base editing system comprising a napDNAbp and a deaminase, the method comprising: using software executing on at least one computer hardware processor to perform: obtaining input data indicative of the nucleotide sequence and a set of one or more guide RNAs; generating first input features from the input data; applying a first machine learning model to the first input features to obtain first output data indicative, for each one guide RNA in the set of guide RNAs, of bystander editing activity, at one or multiple locations in the nucleotide sequence, by the base editing system when using the each one guide RNA; and identifying, using the first output data, the guide RNA for use in the base editing system for introducing the target change into the nucleotide sequence.

Such a method may further comprise an efficiency machine learning model, comprising generating second input features from the input data; applying a second machine learning model to the second input features to obtain second output data indicative, for each one guide RNA in the set of guide RNAs, of a base editing efficiency, at one or multiple locations in the nucleotide sequence, of the base editing system when using the each one guide RNA, wherein identifying the guide RNA is performed using the first output data and the second output data.

In other aspects, the disclosure provides at least one computer readable storage medium storing processor executable instructions that, when executed by at least one computer hardware processor, cause the at least one computer hardware processor to perform a method of identifying a guide RNA for use in a base editing system for introducing a target change into a nucleotide sequence, the base editing system comprising a napDNAbp and a deaminase, the method comprising: obtaining input data indicative of the nucleotide sequence and a set of one or more guide RNAs; generating first input features from the input data; applying a first machine learning model to the first input features to obtain first output data indicative, for each one guide RNA in the set of guide RNAs, of bystander editing activity, at one or multiple locations in the nucleotide sequence, by the base editing system when using the each one guide RNA; and identifying, using the first output data, the guide RNA for use in the base editing system for introducing the target change into the nucleotide sequence.

In still other aspects, the disclosure provides a system, comprising: at least one computer hardware processor; and at least one computer readable storage medium storing processor executable instructions that, when executed by at least one computer hardware processor, cause the at least one computer hardware processor to perform a method of identifying a guide RNA for use in a base editing system for introducing a target change into a nucleotide sequence, the base editing system comprising a napDNAbp and a deaminase, the method comprising: obtaining input data indicative of the nucleotide sequence and a set of one or more guide RNAs; generating first input features from the input data; applying a first machine learning model to the first input features to obtain first output data indicative, for each one guide RNA in the set of guide RNAs, of bystander editing activity, at one or multiple locations in the nucleotide sequence, by the base editing system when using the each one guide RNA; and identifying, using the first output data, the guide RNA for use in the base editing system for introducing the target change into the nucleotide sequence.

The novel machine learning algorithm described and claimed herein can be referred to as “BE-Hive.” The disclosure further provides a graphical user interface that implements BE-Hive, allowing a user to input various features, including a desired target DNA sequence, an appropriate guide RNA (or associated CRISPR protospacer), a base editor, and a cell in which base editing is to take place, and to predict base editing efficiencies and bystander editing patterns for the selected features.

Accordingly, the present disclosure provides a novel machine learning algorithm capable of assisting those of ordinary skill in the art to conduct base editing by, inter alia, facilitating the selection of an appropriate guide RNA and base editor combination which are capable of conducting base editing at a certain level of efficiency and specificity on a given input target DNA sequence desired to be edited to produce an outcome genotype of interest. The novel machine learning algorithm described and claimed herein can be referred to as “BE-Hive.” The disclosure further provides a graphical user interface that implements BE-Hive, allowing a user to input various features, including a desired target DNA sequence, an appropriate guide RNA (or associated CRISPR protospacer), a base editor, and a cell in which base editing is to take place, and to predict base editing efficiencies and bystander editing patterns for the selected features.

It should be appreciated that the foregoing concepts, and additional concepts discussed below, may be arranged in any suitable combination, as the present disclosure is not limited in this respect. Further, other advantages and novel features of the present disclosure will become apparent from the following detailed description of various non-limiting embodiments when considered in conjunction with the accompanying Figures.

### BRIEF DESCRIPTION OF THE DRAWINGS

The following Figures form part of the present specification and are included to further demonstrate certain aspects of the present disclosure, which can be better understood by reference to one or more of these drawings in combination with the detailed description of specific embodiments presented herein.

FIGS. 1A-1I show the systematic characterization of base editing activity at thousands of target sites. FIG. 1A provides an overview of genome-integrated target library assay. Pairs of thousands of sgRNAs and corresponding target sites are integrated into mammalian cells and treated with base editors. Edited cells are enriched by antibiotic selection, and library cassettes are amplified for high-throughput sequencing. FIGS. 1B-1I show base editor activity profiles. Values reflect editing efficiencies of the outcomes specified at the bottom of each heat map, normalized to a maximum of 100, at the protospacer positions shown at each row. Column 3 (C to T) indicates canonical base editing activity (C to T for CBEs and A to G for ABEs), Columns 1-2 indicate other mutation activity at the canonical substrate nucleotide (C for CBEs and A for ABEs), and Columns 4-5 indicates other rare mutations. In the first Column from the left, positions with values ≥50% of maximum are outlined in a box and ≥30% of maximum are shaded.

FIGS. 2A-2I show sequence motifs for base editing outcomes and characterization of indels. FIGS. 2A-2F show sequence motifs for various base editing activities from logistic regression models. The sign of each learned weight indicates a contribution above (positive sign) or below (negative sign) the mean activity. Logo opacity is proportional to the motif's Pearson's R or AUC on held-out sequence contexts. FIG. 2G shows base editing:indel ratio distributions. The table lists geometric mean and interquartile range (IQR). FIG. 2H is a heat map of indel frequencies among edited reads by position and length. Frequencies are normalized (divided) by indel length. FIG. 2I is a heat map of insertion frequencies among all insertions by insert length and number of repeats.

FIGS. 3A-3G show models of base editing efficiency and outcomes. FIG. 3A shows a decision tree for base editing experiment design. To achieve a goal phenotype, such as correcting a pathogenic SNV, a user may enumerate all possible genomic edits, base editors, and sgRNAs that may induce the goal phenotype, and may prioritize these choices with assistance from models that predict base editing efficiency and the frequency of bystander editing patterns that induce the desired phenotype. FIG. 3B shows a model design for predicting base editing efficiency. The input target sequence is featurized and provided to gradient-boosted regression trees which predict a base editing efficiency z-score with an approximately normal distribution centered at 0 with standard deviation 1. Optionally, the user can calibrate the predicted z-score into a predicted fraction of sequenced reads with base editing activity by providing a small amount of data from the user's experimental system. FIG. 3C provides a comparison of predicted versus observed base editing efficiencies at held-out target sites. FIG. 3D shows the design of a deep conditional autoregressive model, a general approach for learning bystander base editing patterns from experimental data. Given a target sequence, sgRNA, base editor, and cell-type, the model generates a combination of editing outcomes at all substrate nucleotides in the target sequence from a probability distribution learned from data. To generate this combination of editing outcomes, the model performs a single generative step per substrate nucleotide, wherein the model generates a predicted editing outcome using the local sequence context around the substrate nucleotide and all previously generated editing outcomes. Once the model has been trained, the model can be queried with any combination of editing outcomes to obtain a predicted frequency among edited reads. FIG. 3E shows the bystander editing model performance at N≥614 held-out target sites. FIG. 3F provides a comparison of predicted versus observed disequilibrium scores, which reflect the tendency of substrate nucleotide pairs to be edited together or separately. Disequilibrium scores equal the predicted or observed probability of both substrate nucleotides edited divided by the probability under the assumption of independent editing events. FIG. 3G shows a diagram of the interactive web application for BE-Hive, which predicts the frequency of bystander editing patterns in the DNA sequence (top) or translated amino acid sequence (bottom). The interactive web application also predicts base editing efficiency.

FIGS. 4A-4H show precise base editing correction of pathogenic alleles. FIG. 4A provides a comparison of predicted versus observed correction precision of disease-related SNVs in mES cells. Trend line depicts rolling mean and standard deviation. FIGS. 4B-4H show the observed frequency of correcting disease-related SNVs to their wild-type genotype among edited reads among varying groups of disease-related SNVs. FIG. 4B shows disease-related SNVs with at least two substrate nucleotides, or any number of substrate nucleotides, in the editing window of each base editor. Error bars depict standard error of the mean. Distribution plot depicts the protospacer positions of SNVs. FIG. 4C shows disease-related SNVs with bystander nucleotides in the editing window of each base editor. FIG. 4D shows disease-related SNVs positioned at C6 with no other bystander nucleotides in the editing window and edited by BE4 in mES cells. FIGS. 4E-4F show disease-related SNVs edited by BE4 (FIG. 4E) and ABE (FIG. 4F). For each subfigure, targets have identical positions of the disease-related SNV and bystander substrate nucleotides in protospacer positions 2-10. Scatter plots compare predicted to observed correction precisions. B=C, G, or T; and D=A, G, or T. FIGS. 4G-4H show disease-related SNVs edited by various base editors. For each subfigure, targets have identical positions of the disease-related SNV and bystander substrate nucleotides in protospacer positions 2-10. Scatter plots compare observed to predicted correction precisions. D=A, G, or T.

FIGS. 5A-5F show sequence determinants of CBE-mediated transversions. FIG. 5A shows sequence motifs for the purity of C editing to A, G, and T. Logo opacity is proportional to the motif's Pearson's R or AUC on held-out sequence contexts. FIG. 5B provides a comparison of average cytosine transversion product purity in mES cells at minimally biased targets versus targets predicted by BE-Hive to be enriched for transversion edits. Error bars depict the standard error of the mean. FIG. 5C shows the relationship between BE:indel ratio and cytosine transversion purity in mES cells. Trend line depicts rolling mean and standard deviation. FIG. 5D shows the relationship between correction precision among edited genotypes and edited amino acid sequences in mES cells. FIG. 5E shows the observed correction precision of disease-related transversion SNVs among edited DNA (lower curve) or edited amino acid sequences (upper curve) in mES cells. FIG. 5F provides a comparison of predicted vs observed correction precision of disease-related transversion mutations by cytosine base editing among edited DNA (left) or edited amino acid sequences (right) in mES cells. Trend lines and shading show the rolling mean and standard deviation, respectively.

FIGS. 6A-6F show that mutations to conserved APOBEC residues increase cytosine transversion purity. FIG. 6A is an evolutionary tree of adenine and cytosine deaminase families. FIG. 6B shows the structural alignment of AID, A3A and homology model of the APOBEC1 deaminase domains by the Theseus software package. Amino acids structurally homologous to T27 or S38 in AID are marked with arrows. FIG. 6C provides a comparison of average transversion purity by eA3A-BE4 and mutant variants and target sequence groups. Error bars show the standard error of the mean. FIG. 6D provides a comparison of average editing efficiency between eA3A-BE4 and mutant variants. Error bars depict standard error of the mean. FIG. 6E shows the observed correction precision of disease-related transversion SNVs among edited DNA (lower curve) or edited amino acid sequences (upper curve) in mES cells. FIG. 6F provides a comparison of predicted versus observed correction precision of disease-related transversion mutations by cytosine base editing among edited DNA (left) or edited amino acid sequences (right) in mES cells. Trend lines and shading show the rolling mean and standard deviation, respectively.

FIGS. 7A-7I show that mutations to conserved APOBEC residues increase CBE product purity. FIGS. 7A-7H show the characterization of EA-BE4 compared to BE4 (FIGS. 7A-7C) and eA3A-BE5 compared to eA3A-BE4 (FIGS. 7D-7F). FIG. 7A and FIG. 7E provide a comparison of transversion frequency by base editor variants with mutations at conserved deaminase residues in BE4 and eA3A-BE4. Error bars depict standard error of the mean. In FIG. 7A, * P<0.02; ** P=2.0×10⁻⁶, N=3,636 and 1,208 substrate nucleotides. 95% CI: 18-35% reduction. In FIG. 7D, * P<0.07; ** P=2.5×10⁵, Welch's T-test, N=1,837 and 685 substrate nucleotides. 95% CI: 17-36% reduction. Welch's T-test was used for each significance test. FIG. 7B and FIG. 7F show base editor mutation activity profiles in HEK293T cells. Values are mean editing efficiencies normalized to a maximum of 100. Protospacer positions with values ≥50% of maximum are outlined and ≥30% of maximum are shaded. FIG. 7C and FIG. 7G show sequence motifs for base editing efficiency in HEK293T cells. FIG. 7D and FIG. 7H provide a comparison of base editing efficiency between BE4 and the EA-BE4 variant, and between eA3A-BE4 and eA3A-BE5. Error bars depict the standard error of the mean. FIG. 7I shows a Pareto frontier depicting the empirical tradeoff between average cytosine transversion purity and editing window size by base editor. Scatter plot densities show bootstrap samples of the mean. Single-nucleotide base editing precision was simulated by choosing the substrate nucleotide closest to the position with maximum base editing efficiency as the target substrate for each base editor. Distribution plot depicts the protospacer position of target nucleotides used in the simulated precision task.

FIGS. 8A-8H show that a genome-integrated library assay is replicable and consistent with endogenous data. FIGS. 8A-8B show average base editing efficiencies by experimental conditions. FIG. 8C shows the consistency of base editing outcome frequencies between biological replicates of the library assay at matched target sites. FIG. 8D shows the consistency of base editing outcome frequencies between data from the library assay versus data from endogenous sites at matched sgRNA-target pairs. FIGS. 8E-8H show base editor mutation activity profiles in HEK293T cells. Values are normalized to a maximum of 100. In the first Column from left, protospacer positions with values ≥50% of maximum are outlined and ≥30% of maximum are shaded.

FIGS. 9A-9L show base editor activity profiles. FIGS. 9A-9L show base editor activity profiles in HEK293T (FIGS. 9A-9D) and U2OS (FIGS. 9E-9L) cells. Values are normalized to a maximum of 100. In the first Column from left, positions with values ≥50% of maximum are outlined and ≥30% of maximum are shaded.

FIGS. 10A-10C show base editing efficiency sequence motifs. FIGS. 10A-10B show sequence motifs for base editing efficiency from logistic regression models. Logo opacity is proportional to the motif's Pearson's R or AUC on held-out sequence contexts. FIG. 10C is a heat map representation of sequence motifs for cytosine base editing efficiency from logistic regression models. Rows depict individual experimental replicates across cell-types and base editors.

FIGS. 11A-11E show the characterization of rare base editing outcomes. FIG. 11A is a heat map representation of sequence motifs for cytosine transversion purity from logistic regression models. Rows depict individual experimental replicates across cell-types and base editors. FIG. 11B shows a fraction of 1-bp indels among all indels, represented by box plots depicting median and interquartile range for various groups of data. Library gold standard conditions were manually defined. FIG. 11C shows a frequency of 1-bp indels by protospacer position. Gold standard conditions have a bimodal distribution peaking at positions 6 and 18, while other library conditions are similar to untreated library conditions with a mostly uniform distribution. FIG. 11D shows the learned parameters from two-way ANOVA performed for adjusting batch effects in observed BE:indel ratios, grouped by cell-type. Horizontal lines indicate the geometric mean. FIG. 11E shows a table of BE:indel ratio statistics with and without 1-bp indel adjustment.

FIGS. 12A-12I show the characterization of base editing indels and modeling of editing outcomes, FIG. 12A is a heat map of indel frequencies among edited reads by position and length. Frequencies are normalized (divided) by indel length. FIG. 12B is a heat map of insertion frequencies among all insertions by insertion length and repeat length. FIG. 12C shows sequence motifs for BE:indel ratios from logistic regression models. Logo opacity is proportional to the motif's Pearson's R or AUC on held-out sequence contexts. Positive logo weights are correlated with higher BE:indel ratios and therefore a lower indel frequency relative to base editing activity. FIG. 12D provides a comparison of BE:indel ratios between experimental replicates of the library assay at matched target sites in mES cells. FIG. 12E shows sequence motifs for base editing efficiency from logistic regression models. Logo opacity is proportional to the motif's Pearson's R or AUC on held-out sequence contexts. Positive logo weights are correlated with higher BE:indel ratios and therefore a lower indel frequency relative to base editing activity. FIGS. 12F-12G show the performance of the gradient-boosted regression tree model at predicting base editing efficiency. Each dot represents a distinct random splitting of data into training and test sets. FIG. 12F shows the performance by training vs test set for each base editor in mES and HEK293T cells. FIG. 12G shows the performance by fraction of training set used, with and without hyperparameter optimization, in mES cells. Trend line is from a lowess model which performs locally weighted linear regression. Trend line was manually extended to “100% with hyperparameter optimization”. FIGS. 12H-12I show the performance of the deep conditional autoregressive model at predicting bystander editing patterns. Each dot represents a distinct random splitting of data into training and test sets. FIG. 12H shows the performance by training versus test set for each base editor in mES and HEK293T cells. FIG. 12I shows the performance by fraction of training set used. Trend line is from a lowess model which performs locally weighted linear regression.

FIGS. 13A-13G show bystander editing model performance. FIG. 13A shows the performance of the deep conditional autoregressive model at predicting bystander editing patterns by the number of substrate nucleotides in protospacer positions 1-12 across all base editors in mES cells. FIG. 13B shows the consistency of observed bystander editing patterns between experimental library replicates at matched target sites by the number of substrate nucleotides in protospacer positions 1-12 across all base editors in mES cells. FIG. 13C shows the observed disequilibrium scores between pairs of substrate nucleotides by the nucleotide distance in mES cells. Disequilibrium scores equal the predicted or observed probability of both substrate nucleotides edited divided by the probability under the assumption of independent editing events. FIG. 13D shows the comparison between observed disequilibrium scores and predicted disequilibrium scores from the deep conditional autoregressive model in mES cells. FIG. 13E shows a comparison of predicted versus observed correction precision of disease-related SNVs in mES cells. Trend line depicts rolling mean and standard deviation. FIGS. 13F-13G show a comparison of predicted versus observed correction precision of disease-related SNVs in HEK293T cells. Trend line depicts rolling mean and standard deviation.

FIGS. 14A-14E show editing outcomes on the transversion-enriched SNV library. FIG. 14A shows the consistency of bystander editing patterns between 35-nt and 61-nt matched target sites by eA3A-BE4 in mES cells. FIG. 14B is a table showing the observed base editing purity of C to A among edited reads by eA3A-BE4 at synthetically optimized target sites in mES cells. FIG. 14C shows sequence motifs for the purity of cytosine editing to adenine, guanine, and thymine by eA3A-BE4, T31A from logistic regression models. Logo opacity is proportional to the motif's Pearson's R or AUC on held-out sequence contexts. Positive logo weights are correlated with higher BE:indel ratios and therefore a lower indel frequency relative to base editing activity. FIG. 14D shows base editing to indel ratio distributions comparing BE4 to EA-BE4. FIG. 14E shows base editing to indel ratio distributions comparing eA3A-BE4 to eA3A-BE5.

FIG. 15 shows adenine base editing at 12,000 sequences in a library context in mESCs.

FIGS. 16A-16C show base editing activity profiles.

FIG. 17 shows base editing preference motifs.

FIG. 18 shows adenine base editing of the SMN2 disease causing SNV in SMA mESCs. Editors denoted below x-axis with PAM sequence in parentheses, and protospacer position of the target nucleotide assuming a 20nt protospacer where the PAM is at position 21-23.

FIG. 19 shows a gel electrophoresis image of SMN cDNA PCR amplification spanning exon 6 to exon 8, depicting bands that include or that have skipped exon 7 in pre-mRNA splicing in SMA mESCs treated with the indicated ABE8-fusion base editors.

FIG. 20 is a graph showing bodyweight in grams of ASO and AAV+ASO treated animals compared to wild type controls (ASO n=3, AAV+ASO n=3, WT n=8).

FIG. 21 is a survival curve of ASO (mean survival 22 days) and AAV+ASO treated animals compared to wild type controls. At time of writing (Jan. 15, 2019) a single AAV treated mouse is still alive at p40.

FIG. 22 shows the time to right after inversion measured in seconds, with a maximum of 30 seconds. Datapoints are averaged across 3 measurements per animal.

FIGS. 23A-23C show open field tests tracing voluntary movement path of wild type (FIGS. 23A-23B) and AAV+ASO treated mutant (FIG. 23C) mice, measured over 20 minutes in light cycle.

FIG. 24A-J provides a series of images (screen shots) of a graphical user interface (GUI) implementation of the machine learning algorithm described herein and referred to as “BE-Hive” and which utilizes only the base editing efficiency machine learning model, as described herein.

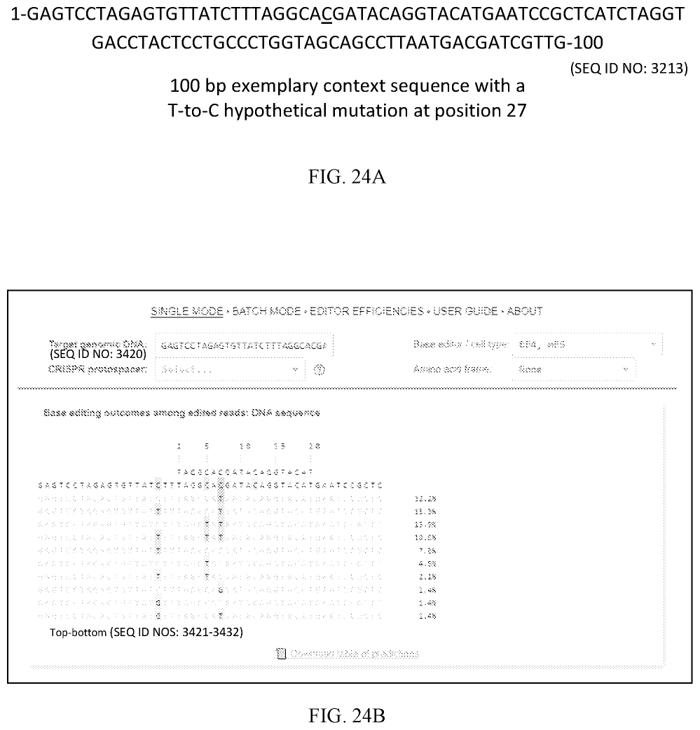

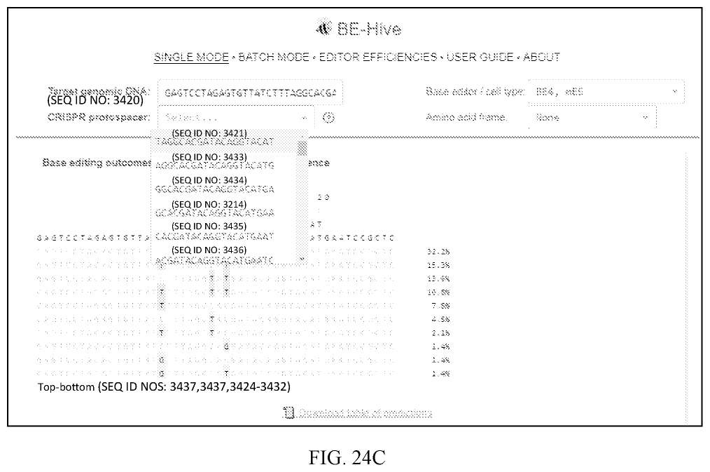

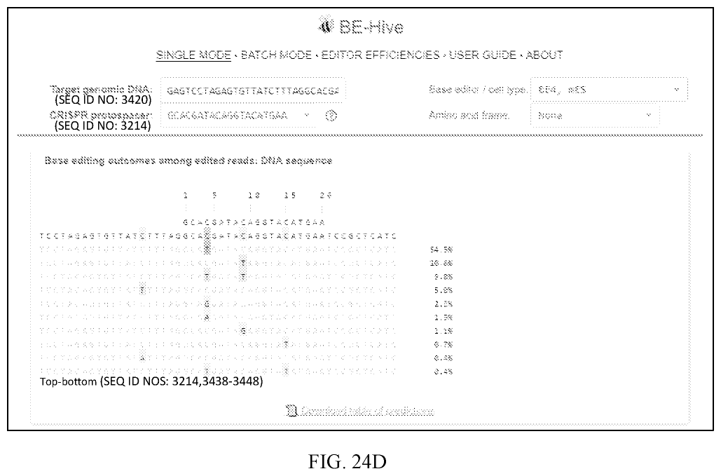

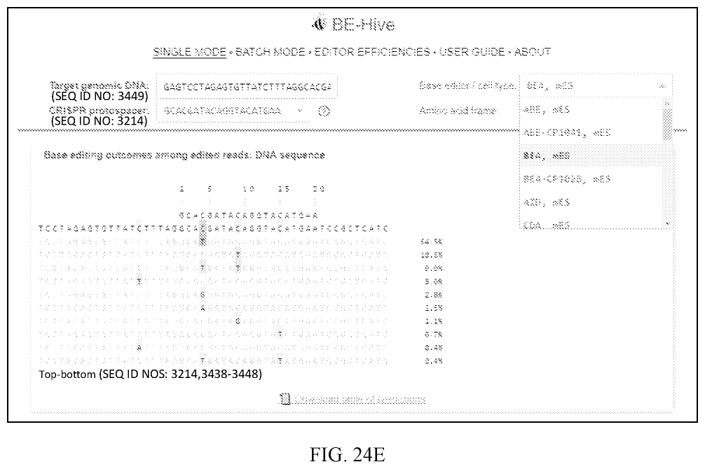

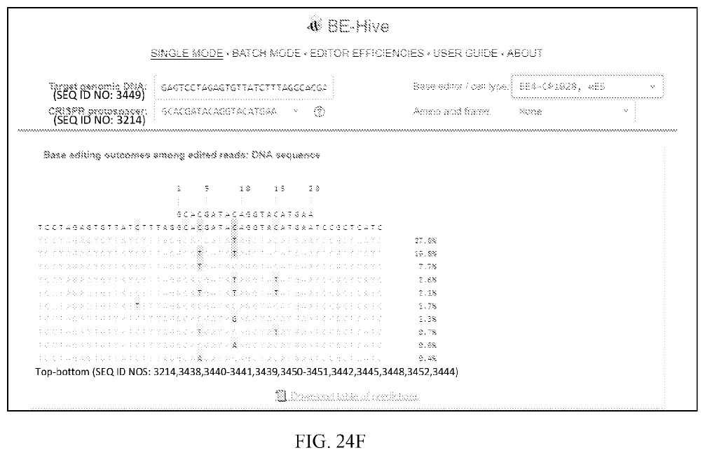

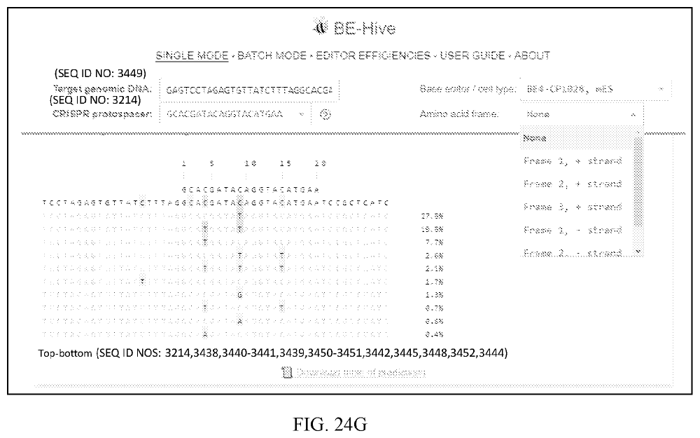

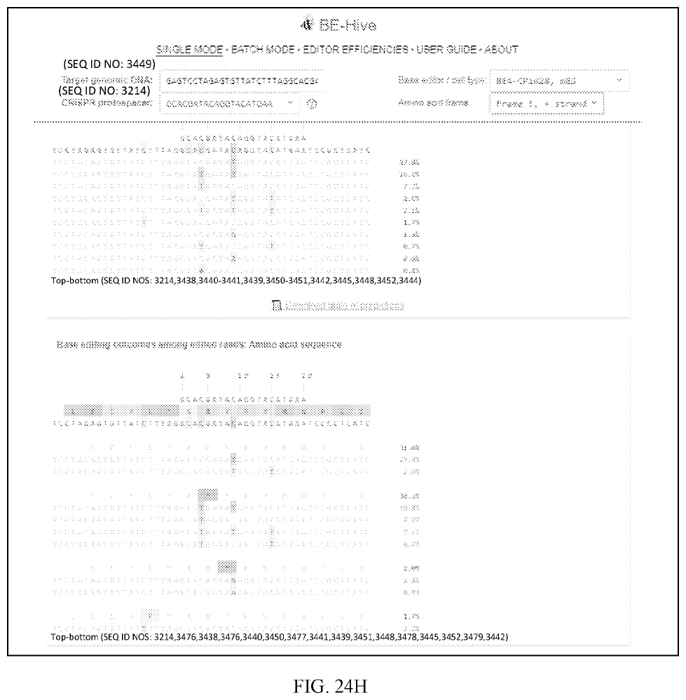

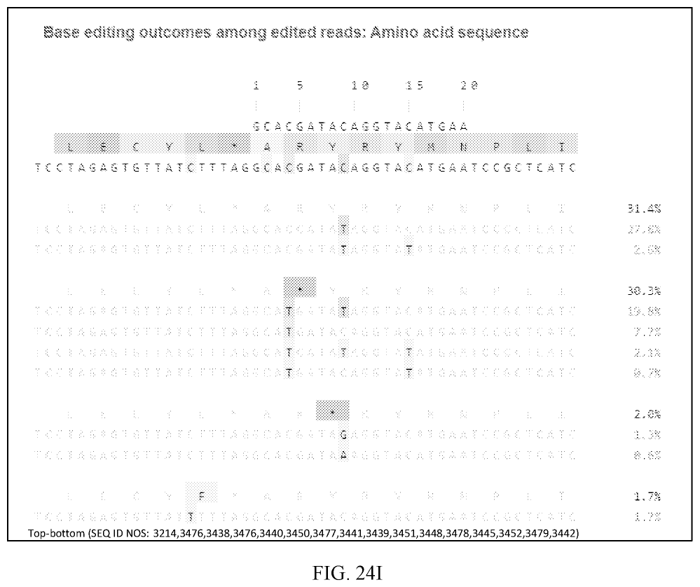

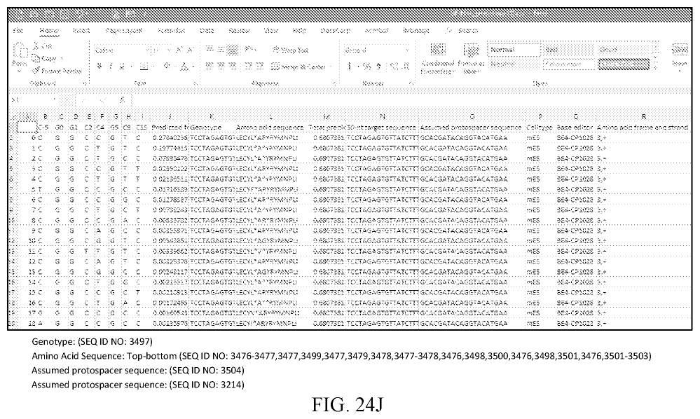

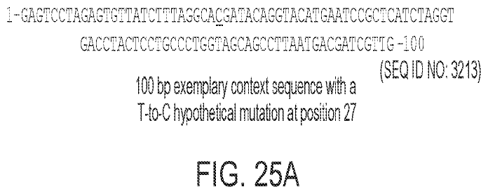

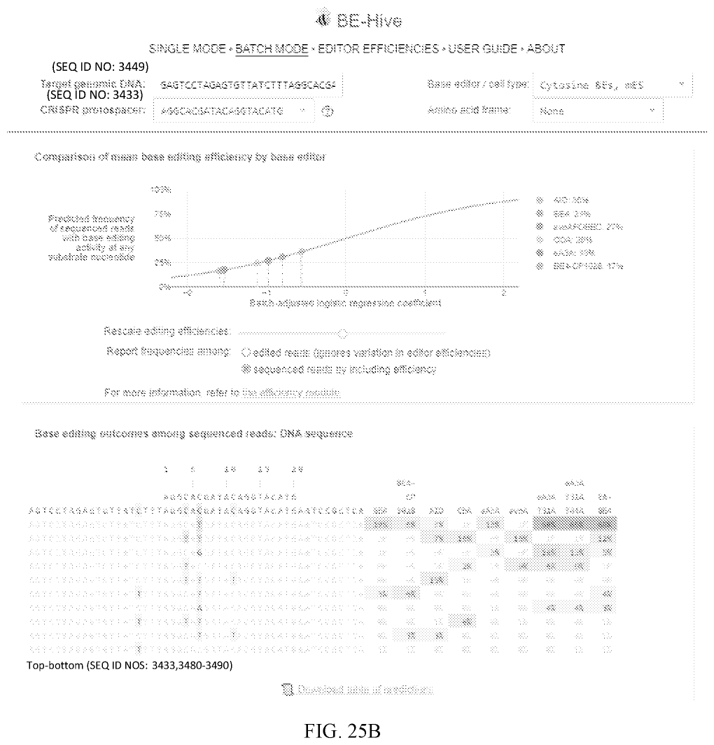

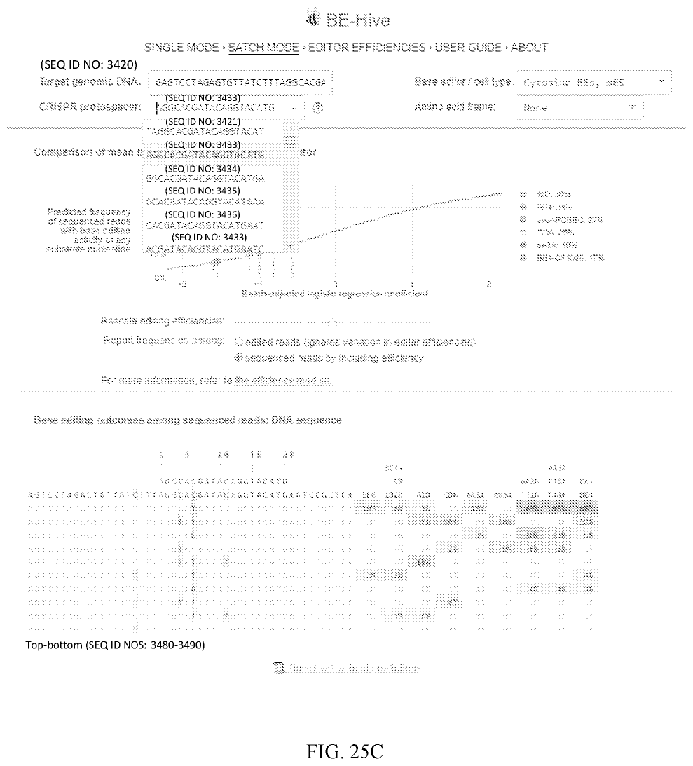

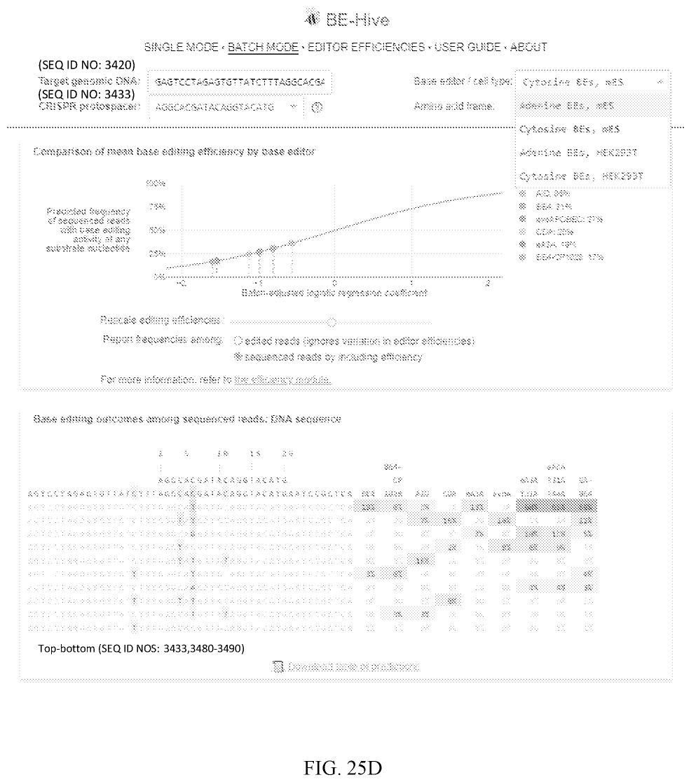

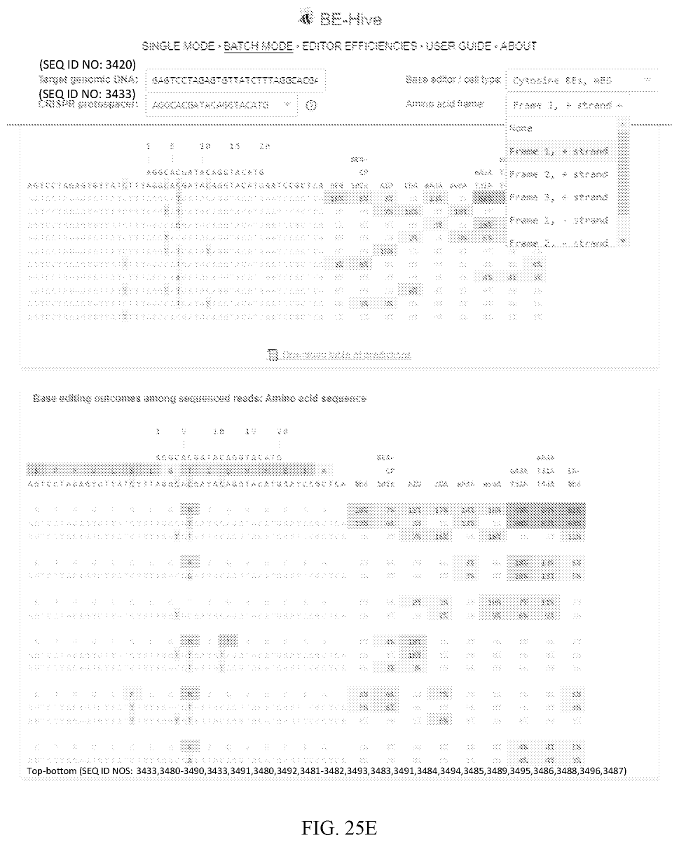

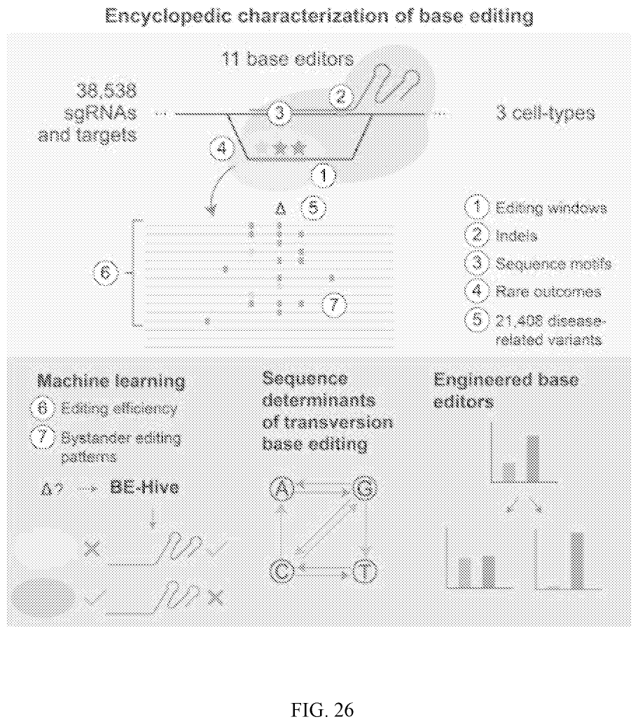

The GUI and underlying algorithm accessed by the GUI assists one of ordinary skill in the art to conduct base editing on a context target sequence of interest. In particular, the embodiment of BE-Hive of FIG. 24A-J utilizes only the base editing efficiency machine learning model. FIG. 24A provides an exemplary context sequence of 100 nucleotides (shown in the 5′-to-3′ direction) and having the sequence GAGTCCTAG AGTGTTATCTTTAGGCACGATACAGGTACATGAATCCGCTCATCTAGGTGACCTA CTCCTGCCCTGGTAGCAGCCTTAATGACGATCGTTG (SEQ ID NO: 3213). The underlined “C” designates a hypothetical T-to-C mutation at position 27, which is desired to be converted back to a T through base editing to eliminate the mutation.

Using a web browser, a user navigates to www.crisprbehive.design and selects “single mode,” as an example of other modes. As shown in FIG. 24B, the user first enters the exemplary context sequence (SEQ ID NO: 3213) into the cell identified as “Target genomic DNA.” The software then populates a set of possible CRISPR protospacers which run along the length of the context sequence as a 20-nt window, beginning at each successive nucleotide position from the 5′-to-3′ direction. FIG. 24C displays the populated set of possible CRISPR protospacers that are generated from the context sequence input as drop-down menu format. The drop-down menu format allows the user to select any specific one protospacer as an input to performing the BE-Hive algorithm. Next, as shown in FIG. 24D and FIG. 24E, the user may also select from a second drop down menu a combination of base editor and cell type. The combination of groups that may be selected are: (1) ABE+mES cells; (2) ABE-CP1041+mES cells; (3) BE4+mES cells; (4) BE4-CP1028+mES cells; (5) AID+mES cells; (6) CDA+mES cells; (7) eA3A+mES cells; (8) evoAPOBEC+mES cells; (9) ABE+HEK293T cells; (10) ABE-CP1041+HEK293T cells; (11) BE4+HEK293T cells; (12) BE4-CP1028+HEK293T cells; (13) AID+HEK293T cells; (14) CDA+HEK293T cells; (15) eA3A+HEK293T cells; (16) evoAPOBEC+HEK293T cells; (17) eA3A-T44DS45A+HEK293T cells; (18) EA-BE4+HEK293T cells; (19) eA3A-T31A+mES cells; (20) eA3A-T31AT44A+mES cells; and (21) EA-BE4+mES cells.

The amino acid sequences of each of the base editor options are provided herein in the Detailed Description. FIG. 24F shows the results for a CRISPR protospacer of GCACGATACAGGTACATGAA (SEQ ID NO: 3214), a base editor of BE4-CP1028, and cell type of mES. The results show the predicted outcomes (ranked as percent efficiencies) of various genotype changes to the target genomic DNA that are possible for the selected combination of the guide RNA (i.e, the protospacer) and the base editor, as predicted by BE-Hive. Thus, in this example, the desired edit of the “C” at position 27 to a “T”, without any bystander changes, only has a predicted efficiency of 7.7%. However, as seen in FIG. 24D, choosing the BE4 base editor in mES cells is predicted to make the desired edit of the “C” at position 27 to a “T” with a 54.5% efficiency. Thus, in this instance, a user would be more inclined—which the particular protospacer choice—to select using the BE4 editor, rather than BE4-CP1028 circular permutant variant.

FIG. 24G permits the user to also input the amino acid frame, which then leads to the prediction by BE-Hive (as shown in FIG. 24H) of base editing outcomes among edited amino acid coding reads present in the context sequence. Thus, with the selection of the BE4-CP1028 editor, a change of a C-to-T at position 27 is predicted to produce a stop codon with a 30.3% efficiency (based on the sum of the individual efficiencies of those genotype outcomes that include said conversion). FIG. 24I is merely a magnified version of the edited amino acid reads. FIG. 24J is the resulting output of the BE-Hive predictions in table form based on the selected inputs.

FIGS. 25A-E provides a series of images (screen shots) of a graphical user interface (GUI) implementation of the machine learning algorithm described and claimed herein and referred to as “BE-Hive” and which utilizes both the base editing efficiency machine learning model and the bystander efficiency machine learning model, as described herein. The GUI and underlying algorithm accessed by the GUI assists one of ordinary skill in the art to conduct base editing on a context target sequence of interest.

FIG. 25A provides an exemplary context sequence of 100 nucleotides (shown in the 5′-to-3′ direction) and having the sequence GAGTCCTAG AGTGTTATCTTTAGGCACGATACAGGTACATGAATCCGCTCATCTAGGTGACCTA CTCCTGCCCTGGTAGCAGCCTTAATGACGATCGTTG (SEQ ID NO: 3213). The underlined “C” designates a hypothetical T-to-C mutation at position 27, which is desired to be converted back to a T through base editing to eliminate the mutation. Using a web browser, a user navigates to www.crisprbehive.design and selects “batch mode.”

As shown in FIG. 25B, the user first enters the exemplary context sequence (SEQ ID NO: 3213) into the cell identified as “Target genomic DNA.” The software then populates a set of possible CRISPR protospacers which run along the length of the context sequence as a 20-nt window, beginning at each successive nucleotide position from the 5′-to-3′ direction. FIG. 25C displays the populated set of possible CRISPR protospacers that are generated from the context sequence input as drop-down menu format. The drop-down menu format allows the user to select any specific one protospacer as an input to performing the BE-Hive algorithm.

Next, as shown in FIG. 25D, the user may also select from a second drop-down menu a combination of base editor and cell type. The combination of groups that may be selected are grouped into four categories: (1) adenine base editors in mES cells; (2) cytosine base editors in mES cells; (3) adenine base editors in HEK293T cells; and (4) cytosine base editors in HEK293T cells.

Once selected, the BE-Hive algorithm processes the inputs (the selected protospacer and the selected base editor/cell type) and displays the output in the form of a table entitled “Base editing outcomes among sequenced reads: DNA sequence.” This table displays the selected protospacer at the top row and the Target genomic DNA sequence in the second row from the top. The protospacer is aligned over its corresponding position in the Target genomic DNA sequence. The remaining rows each display a corresponding genotype outcome, and shows with yellow highlighting those nucleotide changes that would result by base editing with said inputs. At the rightmost side are two columns, each displaying the percentage of efficiency of introducing the designated edit in yellow highlighting, wherein each column provides the efficiency data for each of the available base editors in the selected category. For example, in the selected category of “Adenine BEs, mES)” in the drop-down menu, the output columns of base editors include, from left to right, ABE and ABE CP1041.

In the selected category of “Cytosine BEs, mES” in the drop-down menu, the output columns of base editors include, from left to right, BE4, BE4 CP1028, AID, CDA, eA3A, evoA, eA3A T31A, eA3A T31A T44A, and EA-BE4 (as shown in FIG. 25D). In addition, as shown in FIG. 25D, for each genotype outcome, the percent efficiency for each specific base editor is shown. To demonstrate, for the first genotype outcome-which makes the desired C-to-T conversion at position 27 of the Target genomic DNA—the base editor, BE4, has a predicted efficiency of 19%. By contrast, AID only has a predicted efficiency of 3%. And, the eA3A T31A and eA3A T31AT44A editors each have a higher predicted efficiency of 68% and 65%, respectively.

In addition, as shown in FIG. 25E, the user may also focus the prediction of the algorithm on predicting the efficiency of producing certain amino acid residue outcomes within each of the six possible reading frames along the length of the Target genomic DNA. For example, the first row of amino acid sequence showing a Met (“M”) in place of the Thr (“T”) in the starting amino acid sequence (top row) represents the first possible modified amino acid sequence outcome. This outcome is associated with two different possible genotype outcomes, including one which converts the target C to a T at position 27 of the Target genomic DNA. The columns at the right most side provide the predicted efficiency of converting a Thr (“T”) to an Met (“M”) the indicate position for each of the listed base editors (in this case, the cytosine base editors).

FIG. 26 provides a schematic that represents the use of BE-Hive to facilitate base editing.

### DEFINITIONS

As used herein and in the claims, the singular forms “a,” “an,” and “the” include the singular and the plural reference unless the context clearly indicates otherwise. Thus, for example, a reference to “an agent” includes a single agent and a plurality of such agents.

### AAV

An “adeno-associated virus” or “AAV” is a virus which infects humans and some other primate species. The wild-type AAV genome is a single-stranded deoxyribonucleic acid (ssDNA), either positive- or negative-sensed. The genome comprises two inverted terminal repeats (ITRs), one at each end of the DNA strand, and two open reading frames (ORFs): rep and cap between the ITRs. The rep ORF comprises four overlapping genes encoding Rep proteins required for the AAV life cycle. The cap ORF comprises overlapping genes encoding capsid proteins: VP1, VP2 and VP3, which interact together to form the viral capsid. VP1, VP2 and VP3 are translated from one mRNA transcript, which can be spliced in two different manners: either a longer or shorter intron can be excised resulting in the formation of two isoforms of mRNAs: a ˜2.3 kb- and a ˜2.6 kb-long mRNA isoform. The capsid forms a supramolecular assembly of approximately 60 individual capsid protein subunits into a non-enveloped, T-1 icosahedral lattice capable of protecting the AAV genome. The mature capsid is composed of VP1, VP2, and VP3 (molecular masses of approximately 87, 73, and 62 kDa respectively) in a ratio of about 1:1:10.

rAAV particles may comprise a nucleic acid vector (e.g., a recombinant genome), which may comprise at a minimum: (a) one or more heterologous nucleic acid regions comprising a sequence encoding a protein or polypeptide of interest (e.g., a split Cas9 or split nucleobase) or an RNA of interest (e.g., a gRNA), or one or more nucleic acid regions comprising a sequence encoding a Rep protein; and (b) one or more regions comprising inverted terminal repeat (ITR) sequences (e.g., wild-type ITR sequences or engineered ITR sequences) flanking the one or more nucleic acid regions (e.g., heterologous nucleic acid regions). In some embodiments, the nucleic acid vector is between 4 kb and 5 kb in size (e.g., 4.2 to 4.7 kb in size). In some embodiments, the nucleic acid vector further comprises a region encoding a Rep protein. In some embodiments, the nucleic acid vector is circular. In some embodiments, the nucleic acid vector is single-stranded. In some embodiments, the nucleic acid vector is double-stranded. In some embodiments, a double-stranded nucleic acid vector may be, for example, a self-complimentary vector that contains a region of the nucleic acid vector that is complementary to another region of the nucleic acid vector, initiating the formation of the double-strandedness of the nucleic acid vector.

### Adenosine Deaminase (or Adenine Deaminase)

As used herein, the term “adenosine deaminase” or “adenosine deaminase domain” refers to a protein or enzyme that catalyzes a deamination reaction of an adenosine (or adenine). The terms “adenosine” and “adenine” are used interchangeably for purposes of the present disclosure. For example, for purposes of the disclosure, reference to an “adenine base editor” (ABE) refers to the same entity as an “adenosine base editor” (ABE). Similarly, for purposes of the disclosure, reference to an “adenine deaminase” refers to the same entity as an “adenosine deaminase.” However, the person having ordinary skill in the art will appreciate that “adenine” refers to the purine base whereas “adenosine” refers to the larger nucleoside molecule that includes the purine base (adenine) and sugar moiety (e.g., either ribose or deoxyribose). In certain embodiments, the disclosure provides base editor fusion proteins comprising one or more adenosine deaminase domains. For instance, an adenosine deaminase domain may comprise a heterodimer of a first adenosine deaminase and a second deaminase domain, connected by a linker. Adenosine deaminases (e.g., engineered adenosine deaminases or evolved adenosine deaminases) provided herein may be enzymes that convert adenine (A) to inosine (I) in DNA or RNA. Such adenosine deaminase can lead to an A:T to G:C base pair conversion. In some embodiments, the deaminase is a variant of a naturally-occurring deaminase from an organism. In some embodiments, the deaminase does not occur in nature. For example, in some embodiments, the deaminase is at least 50%, at least 55%, at least 60%, at least 65%, at least 70%, at least 75% at least 80%, at least 85%, at least 90%, at least 95%, at least 96%, at least 97%, at least 98%, at least 99%, or at least 99.5% identical to a naturally-occurring deaminase.

In some embodiments, the adenosine deaminase is derived from a bacterium, such as, E. coli, S. aureus, S. typhi, S. putrefaciens, H. influenzae, or C. crescentus. In some embodiments, the adenosine deaminase is a TadA deaminase. In some embodiments, the TadA deaminase is an E. coli TadA deaminase (ecTadA). In some embodiments, the TadA deaminase is a truncated E. coli TadA deaminase. For example, the truncated ecTadA may be missing one or more N-terminal amino acids relative to a full-length ecTadA. In some embodiments, the truncated ecTadA may be missing 1, 2, 3, 4, 5, 6, 7, 8, 9, 10, 11, 12, 13, 14, 15, 6, 17, 18, 19, or 20 N-terminal amino acid residues relative to the full length ecTadA. In some embodiments, the truncated ecTadA may be missing 1, 2, 3, 4, 5, 6, 7, 8, 9, 10, 11, 12, 13, 14, 15, 6, 17, 18, 19, or 20 C-terminal amino acid residues relative to the full length ecTadA. In some embodiments, the ecTadA deaminase does not comprise an N-terminal methionine. Reference is made to U.S. Patent Publication No. 2018/0073012, published Mar. 15, 2018, which is incorporated herein by reference.

### Antisense Strand

In genetics, the “antisense” strand of a segment within double-stranded DNA is the template strand, and which is considered to run in the 3′ to 5′ orientation. By contrast, the “sense” strand is the segment within double-stranded DNA that runs from 5′ to 3′, and which is complementary to the antisense strand of DNA, or template strand, which runs from 3′ to 5′. In the case of a DNA segment that encodes a protein, the sense strand is the strand of DNA that has the same sequence as the mRNA, which takes the antisense strand as its template during transcription, and eventually undergoes (typically, not always) translation into a protein. The antisense strand is thus responsible for the RNA that is later translated to protein, while the sense strand possesses a nearly identical makeup to that of the mRNA. Note that for each segment of dsDNA, there will possibly be two sets of sense and antisense, depending on which direction one reads (since sense and antisense is relative to perspective). It is ultimately the gene product, or mRNA, that dictates which strand of one segment of dsDNA is referred to as sense or antisense.

### Base Editing

“Base editing” refers to genome editing technology that involves the conversion of a specific nucleic acid base into another at a targeted genomic locus. In certain embodiments, this can be achieved without requiring double-stranded DNA breaks (DSB), or single stranded breaks (i.e., nicking). To date, other genome editing techniques, including CRISPR-based systems, begin with the introduction of a DSB at a locus of interest. Subsequently, cellular DNA repair enzymes mend the break, commonly resulting in random insertions or deletions (indels) of bases at the site of the DSB. However, when the introduction or correction of a point mutation at a target locus is desired rather than stochastic disruption of the entire gene, these genome editing techniques are unsuitable, as correction rates are low (e.g. typically 0.1% to 5%), with the major genome editing products being indels. In order to increase the efficiency of gene correction without simultaneously introducing random indels, the present inventors previously modified the CRISPR/Cas9 system to directly convert one DNA base into another without DSB formation. See, Komor, A. C., et al., Programmable editing of a target base in genomic DNA without double-stranded DNA cleavage. Nature 533, 420-424 (2016), the entire contents of which is incorporated by reference herein.

### Base Editor

The term “base editor (BE)” as used herein, refers to an agent comprising a polypeptide that is capable of making a modification to a base (e.g., A, T, C, G, or U) within a nucleic acid sequence (e.g., DNA or RNA) that converts one base to another (e.g., A to G, A to C, A to T, C to T, C to G, C to A, G to A, G to C, G to T, T to A, T to C, T to G). In some embodiments, the base editor is capable of deaminating a base within a nucleic acid such as a base within a DNA molecule. In the case of an adenine base editor, the base editor is capable of deaminating an adenine (A) in DNA. Such base editors may include a nucleic acid programmable DNA binding protein (napDNAbp) fused to an adenosine deaminase. Some base editors include CRISPR-mediated fusion proteins that are utilized in the base editing methods described herein. In some embodiments, the base editor comprises a nuclease-inactive Cas9 (dCas9) fused to a deaminase which binds a nucleic acid in a guide RNA-programmed manner via the formation of an R-loop, but does not cleave the nucleic acid. For example, the dCas9 domain of the fusion protein may include a D10A and a H840A mutation (which renders Cas9 capable of cleaving only one strand of a nucleic acid duplex), as described in PCT/US2016/058344, which published as WO 2017/070632 on Apr. 27, 2017, and is incorporated herein by reference in its entirety. The DNA cleavage domain of S. pyogenes Cas9 includes two subdomains, the HNH nuclease subdomain and the RuvC1 subdomain. The HNH subdomain cleaves the strand complementary to the gRNA (the “targeted strand”, or the strand in which editing or deamination occurs), whereas the RuvC1 subdomain cleaves the non-complementary strand containing the PAM sequence (the “non-edited strand”). The RuvC1 mutant D10A generates a nick in the targeted strand, while the HNH mutant H840A generates a nick on the non-edited strand (see Jinek et al., Science, 337:816-821(2012); Qi et al., Cell. 28; 152(5):1173-83 (2013)).

In some embodiments, a nucleobase editor is a macromolecule or macromolecular complex that results primarily (e.g., more than 80%, more than 85%, more than 90%, more than 95%, more than 99%, more than 99.9%, or 100%) in the conversion of a nucleobase in a polynucleic acid sequence into another nucleobase (i.e., a transition or transversion) using a combination of 1) a nucleotide-, nucleoside-, or nucleobase-modifying enzyme; and 2) a nucleic acid binding protein that can be programmed to bind to a specific nucleic acid sequence.

In some embodiments, the nucleobase editor comprises a DNA binding domain (e.g., a programmable DNA binding domain such as a dCas9 or nCas9) that directs it to a target sequence. In some embodiments, the nucleobase editor comprises a nucleobase modifying enzyme fused to a programmable DNA binding domain (e.g., a dCas9 or nCas9). A “nucleobase modifying enzyme” is an enzyme that can modify a nucleobase and convert one nucleobase to another (e.g., a deaminase such as a cytidine deaminase or a adenosine deaminase). In some embodiments, the nucleobase editor may target cytosine (C) bases in a nucleic acid sequence and convert the C to thymine (T) base. In some embodiments, the C to T editing is carried out by a deaminase, e.g., a cytidine deaminase. Base editors that can carry out other types of base conversions (e.g., adenosine (A) to guanine (G), C to G) are also contemplated.

Nucleobase editors that convert a C to T, in some embodiments, comprise a cytidine deaminase. A “cytidine deaminase” refers to an enzyme that catalyzes the chemical reaction “cytosine+H₂O→uracil+NH₃” or “5-methyl-cytosine+H₂O→thymine+NH₃.” As it may be apparent from the reaction formula, such chemical reactions result in a C to U/T nucleobase change. In the context of a gene, such a nucleotide change, or mutation, may in turn lead to an amino acid change in the protein, which may affect the protein's function, e.g., loss-of-function or gain-of-function. In some embodiments, the C to T nucleobase editor comprises a dCas9 or nCas9 fused to a cytidine deaminase. In some embodiments, the cytidine deaminase domain is fused to the N-terminus of the dCas9 or nCas9. In some embodiments, the nucleobase editor further comprises a domain that inhibits uracil glycosylase, and/or a nuclear localization signal. Such nucleobase editors have been described in the art, e.g., in Rees & Liu, Nat Rev Genet. 2018; 19(12):770-788 and Koblan et al., Nat Biotechnol. 2018; 36(9):843-846; as well as. U.S. Patent Publication No. 2018/0073012, published Mar. 15, 2018, which issued as U.S. Pat. No. 10,113,163; on Oct. 30, 2018; U.S. Patent Publication No. 2017/0121693, published May 4, 2017, which issued as U.S. Pat. No. 10,167,457 on Jan. 1, 2019; International Publication No. WO 2017/070633, published Apr. 27, 2017; U.S. Patent Publication No. 2015/0166980, published Jun. 18, 2015; U.S. Pat. No. 9,840,699, issued Dec. 12, 2017; U.S. Pat. No. 10,077,453, issued Sep. 18, 2018; International Publication No. WO 2019/023680, published Jan. 31, 2019; International Publication No. WO 2018/0176009, published Sep. 27, 2018, International Application No PCT/US2019/033848, filed May 23, 2019, International Application No. PCT/US2019/47996, filed Aug. 23, 2019; International Application No. PCT/US2019/049793, filed Sep. 5, 2019; U.S. Provisional Application No. 62/835,490, filed Apr. 17, 2019; International Application No. PCT/US2019/61685, filed Nov. 15, 2019; International Application No. PCT/US2019/57956, filed Oct. 24, 2019; U.S. Provisional Application No. 62/858,958, filed Jun. 7, 2019; International Publication No. PCT/US2019/58678, filed Oct. 29, 2019, the contents of each of which are incorporated herein by reference in their entireties.

In some embodiments, a nucleobase editor converts an A to G. In some embodiments, the nucleobase editor comprises an adenosine deaminase. An “adenosine deaminase” is an enzyme involved in purine metabolism. It is needed for the breakdown of adenosine from food and for the turnover of nucleic acids in tissues. Its primary function in humans is the development and maintenance of the immune system. An adenosine deaminase catalyzes hydrolytic deamination of adenosine (forming inosine, which base pairs as G) in the context of DNA. There are no known adenosine deaminases that act on DNA. Instead, known adenosine deaminase enzymes only act on RNA (tRNA or mRNA). Evolved deoxyadenosine deaminase enzymes that accept DNA substrates and deaminate dA to deoxyinosine have been described, e.g., in PCT Application PCT/US2017/045381, filed Aug. 3, 2017, which published as WO 2018/027078, and PCT Application No. PCT/US2019/033848, which published as WO 2019/226953, each of which is herein incorporated by reference by reference.

Exemplary adenine and cytosine base editors are also described in Rees & Liu, Base editing: precision chemistry on the genome and transcriptome of living cells, Nat. Rev. Genet. 2018; 19(12):770-788; as well as U.S. Patent Publication No. 2018/0073012, published Mar. 15, 2018, which issued as U.S. Pat. No. 10,113,163, on Oct. 30, 2018; U.S. Patent Publication No. 2017/0121693, published May 4, 2017, which issued as U.S. Pat. No. 10,167,457 on Jan. 1, 2019; International Publication No. WO 2017/070633, published Apr. 27, 2017; U.S. Patent Publication No. 2015/0166980, published Jun. 18, 2015; U.S. Pat. No. 9,840,699, issued Dec. 12, 2017; and U.S. Pat. No. 10,077,453, issued Sep. 18, 2018, the contents of each of which are incorporated herein by reference in their entireties.

The term “evolved base editor” or “evolved base editor variant” refers to a base editor formed as a result of mutagenizing a reference or starting-point base editor. The term refers to embodiments in which the nucleotide modification domain is evolved or a separate domain is evolved. Mutagenizing a reference (or starting-point) base editor may comprise mutagenizing an adenosine deaminase. Amino acid sequence variations may include one or more mutated residues within the amino acid sequence of a reference base editor, e.g., as a result of a change in the nucleotide sequence encoding the base editor that results in a change in the codon at any particular position in the coding sequence, the deletion of one or more amino acids (e.g., a truncated protein), the insertion of one or more amino acids, or any combination of the foregoing. The evolved base editor may include variants in one or more components or domains of the base editor (e.g., mutations introduced into one or more adenosine deaminases).

### Cas9

The term “Cas9” or “Cas9 nuclease” refers to an RNA-guided nuclease comprising a Cas9 domain, or a fragment thereof (e.g., a protein comprising an active or inactive DNA cleavage domain of Cas9, and/or the gRNA binding domain of Cas9). A “Cas9 domain” as used herein, is a protein fragment comprising an active or inactive cleavage domain of Cas9 and/or the gRNA binding domain of Cas9. A “Cas9 protein” is a full length Cas9 protein. A Cas9 nuclease is also referred to sometimes as a casn1 nuclease or a CRISPR (Clustered Regularly Interspaced Short Palindromic Repeat)-associated nuclease. CRISPR is an adaptive immune system that provides protection against mobile genetic elements (viruses, transposable elements, and conjugative plasmids). CRISPR clusters contain spacers, sequences complementary to antecedent mobile elements, and target invading nucleic acids. CRISPR clusters are transcribed and processed into CRISPR RNA (crRNA). In type II CRISPR systems correct processing of pre-crRNA requires a trans-encoded small RNA (tracrRNA), endogenous ribonuclease 3 (rnc) and a Cas9 domain. The tracrRNA serves as a guide for ribonuclease 3-aided processing of pre-crRNA. Subsequently, Cas9/crRNA/tracrRNA endonucleolytically cleaves linear or circular dsDNA target complementary to the spacer. The target strand not complementary to crRNA is first cut endonucleolytically, then trimmed 3′-5′ exonucleolytically. In nature, DNA-binding and cleavage typically requires protein and both RNAs. However, single guide RNAs (“sgRNA”, or simply “gNRA”) can be engineered so as to incorporate aspects of both the crRNA and tracrRNA into a single RNA species. See, e.g., Jinek M., Chylinski K., Fonfara I., Hauer M., Doudna J. A., Charpentier E. Science 337:816-821(2012), the entire contents of which are hereby incorporated by reference. Cas9 recognizes a short motif in the CRISPR repeat sequences (the PAM or protospacer adjacent motif) to help distinguish self versus non-self. Cas9 nuclease sequences and structures are well known to those of skill in the art (see, e.g., “Complete genome sequence of an M1 strain of Streptococcus pyogenes.” Ferretti et al., J. J., McShan W. M., Ajdic D. J., Savic D. J., Savic G., Lyon K., Primeaux C., Sezate S., Suvorov A. N., Kenton S., Lai H. S., Lin S. P., Qian Y., Jia H. G., Najar F. Z., Ren Q., Zhu H., Song L., White J., Yuan X., Clifton S. W., Roe B. A., McLaughlin R E., Proc. Natl. Acad. Sci. U.S.A. 98:4658-4663(2001); “CRISPR RNA maturation by trans-encoded small RNA and host factor RNase III.” Deltcheva E., Chylinski K., Sharma C. M., Gonzales K., Chao Y., Pirzada Z. A., Eckert M. R., Vogel J., Charpentier E., Nature 471:602-607(2011); and “A programmable dual-RNA-guided DNA endonuclease in adaptive bacterial immunity.” Jinek M., Chylinski K., Fonfara I., Hauer M., Doudna J. A., Charpentier E. Science 337:816-821(2012), the entire contents of each of which are incorporated herein by reference). Cas9 orthologs have been described in various species, including, but not limited to, S. pyogenes and S. thermophilus. Additional suitable Cas9 nucleases and sequences will be apparent to those of skill in the art based on this disclosure, and such Cas9 nucleases and sequences include Cas9 sequences from the organisms and loci disclosed in Chylinski, Rhun, and Charpentier, “The tracrRNA and Cas9 families of type II CRISPR-Cas immunity systems” (2013) RNA Biology 10:5, 726-737; the entire contents of which are incorporated herein by reference. In some embodiments, a Cas9 nuclease comprises one or more mutations that partially impair or inactivate the DNA cleavage domain.

A nuclease-inactivated Cas9 domain may interchangeably be referred to as a “dCas9” protein (for nuclease-“dead” Cas9). Methods for generating a Cas9 domain (or a fragment thereof) having an inactive DNA cleavage domain are known (see, e.g., Jinek et al., Science. 337:816-821(2012); Qi et al., “Repurposing CRISPR as an RNA-Guided Platform for Sequence-Specific Control of Gene Expression” (2013) Cell. 28; 152(5):1173-83, the entire contents of each of which are incorporated herein by reference). For example, the DNA cleavage domain of Cas9 is known to include two subdomains, the HNH nuclease subdomain and the RuvC1 subdomain. The HNH subdomain cleaves the strand complementary to the gRNA, whereas the RuvC1 subdomain cleaves the non-complementary strand. Mutations within these subdomains can silence the nuclease activity of Cas9. For example, the mutations D10A and H840A completely inactivate the nuclease activity of S. pyogenes Cas9 (Jinek et al., Science. 337:816-821(2012); Qi et al., Cell. 28; 152(5):1173-83 (2013)). In some embodiments, proteins comprising fragments of Cas9 are provided. For example, in some embodiments, a protein comprises one of two Cas9 domains: (1) the gRNA binding domain of Cas9; or (2) the DNA cleavage domain of Cas9. In some embodiments, proteins comprising Cas9 or fragments thereof are referred to as “Cas9 variants.” A Cas9 variant shares homology to Cas9, or a fragment thereof. For example, a Cas9 variant is at least about 70% identical, at least about 80% identical, at least about 90% identical, at least about 95% identical, at least about 96% identical, at least about 97% identical, at least about 98% identical, at least about 99% identical, at least about 99.5% identical, at least about 99.8% identical, or at least about 99.9% identical to wild type Cas9 (e.g., SpCas9 of SEQ ID NO: 5). In some embodiments, the Cas9 variant may have 1, 2, 3, 4, 5, 6, 7, 8, 9, 10, 11, 12, 13, 14, 15, 16, 17, 18, 19, 20, 21, 22, 21, 24, 25, 26, 27, 28, 29, 30, 31, 32, 33, 34, 35, 36, 37, 38, 39, 40, 41, 42, 43, 44, 45, 46, 47, 48, 49, 50, or more amino acid changes compared to wild type Cas9 (e.g., SpCas9 of SEQ ID NO: 5). In some embodiments, the Cas9 variant comprises a fragment of Cas9 (e.g., a gRNA binding domain or a DNA-cleavage domain), such that the fragment is at least about 70% identical, at least about 80% identical, at least about 90% identical, at least about 95% identical, at least about 96% identical, at least about 97% identical, at least about 98% identical, at least about 99% identical, at least about 99.5% identical, or at least about 99.9% identical to the corresponding fragment of wild type Cas9 (e.g., SpCas9 of SEQ ID NO: 5). In some embodiments, the fragment is at least 30%, at least 35%, at least 40%, at least 45%, at least 50%, at least 55%, at least 60%, at least 65%, at least 70%, at least 75%, at least 80%, at least 85%, at least 90%, at least 95% identical, at least 96%, at least 97%, at least 98%, at least 99%, or at least 99.5% of the amino acid length of a corresponding wild type Cas9 (e.g., SpCas9 of SEQ ID NO: 5).

As used herein, the term “nCas9” or “Cas9 nickase” refers to a Cas9 or a variant thereof, which cleaves or nicks only one of the strands of a target cut site thereby introducing a nick in a double strand DNA molecule rather than creating a double strand break. This can be achieved by introducing appropriate mutations in a wild-type Cas9 which inactivates one of the two endonuclease activities of the Cas9. Any suitable mutation which inactivates one Cas9 endonuclease activity but leaves the other intact is contemplated, such as one of D10A or H840A mutations in the wild-type S. pyogenes Cas9 amino acid sequence, or a D10A mutation in the wild-type S. aureus Cas9 amino acid sequence, may be used to form the nCas9.

cDNA

The term “cDNA” refers to a strand of DNA copied from an RNA template. cDNA is complementary to the RNA template.

### Circular Permutant

As used herein, the term “circular permutant” refers to a protein or polypeptide (e.g., a Cas9) comprising a circular permutation, which is change in the protein's structural configuration involving a change in order of amino acids appearing in the protein's amino acid sequence. In other words, circular permutants are proteins that have altered N- and C-termini as compared to a wild-type counterpart, e.g., the wild-type C-terminal half of a protein becomes the new N-terminal half. Circular permutation (or CP) is essentially the topological rearrangement of a protein's primary sequence, connecting its N- and C-terminus, often with a peptide linker, while concurrently splitting its sequence at a different position to create new, adjacent N- and C-termini. The result is a protein structure with different connectivity, but which often can have the same overall similar three-dimensional (3D) shape, and possibly include improved or altered characteristics, including, reduced proteolytic susceptibility, improved catalytic activity, altered substrate or ligand binding, and/or improved thermostability. Circular permutant proteins can occur in nature (e.g., concanavalin A and lectin). In addition, circular permutation can occur as a result of posttranslational modifications or may be engineered using recombinant techniques (e.g., see, Oakes et al., “Protein Engineering of Cas9 for enhanced function,” Methods Enzymol, 2014, 546: 491-511 and Oakes et al., “CRISPR-Cas9 Circular Permutants as Programmable Scaffolds for Genome Modification,” Cell, Jan. 10, 2019, 176: 254-267, each of are incorporated herein by reference).

Circularly Permuted napDNAbp

The term “circularly permuted napDNAbp” refers to any napDNAbp protein, or variant thereof (e.g., SpCas9), that occurs as or engineered as a circular permutant, whereby its N- and C-termini have been topically rearranged. Such circularly permuted proteins (“CP-napDNAbp”, such as “CP-Cas9” in the case of Cas9), or variants thereof, retain the ability to bind DNA when complexed with a guide RNA (gRNA). See, Oakes et al., “Protein Engineering of Cas9 for enhanced function,” Methods Enzymol, 2014, 546: 491-511 and Oakes et al., “CRISPR-Cas9 Circular Permutants as Programmable Scaffolds for Genome Modification,” Cell, Jan. 10, 2019, 176: 254-267, each of are incorporated herein by reference. The instant disclosure contemplates any previously known CP-Cas9 or use a new CP-Cas9 so long as the resulting circularly permuted protein retains the ability to bind DNA when complexed with a guide RNA (gRNA).

### Cytidine Deaminase (or Cytosine Deaminase)

As used herein, the term “cytidine deaminase” or “cytidine deaminase domain” refers to a protein or enzyme that catalyzes a deamination reaction of a cytidine or cytosine. The terms “cytidine” and “cytosine” are used interchangeably for purposes of the present disclosure. For example, for purposes of the disclosure, reference to an “cytidine base editor” (CBE) refers to the same entity as an “cytosine base editor” (CBE). Similarly, for purposes of the disclosure, reference to an “cytidine deaminase” refers to the same entity as an “cytosine deaminase.” However, the person having ordinary skill in the art will appreciate that “cytosine” refers to the pyrimidine base whereas “cytidine” refers to the larger nucleoside molecule that includes the pyrimidine base (cytosine) and sugar moiety (e.g., either ribose or deoxyribose). A cytidine deaminase is encoded by the CDA gene and is an enzyme that catalyzes the removal of an amine group from cytidine (i.e., the base cytosine when attached to a ribose ring, i.e., the nucleoside referred to as cytidine) to uridine (C to U) and deoxycytidine to deoxyuridine (C to U). A non-limiting example of a cytidine deaminase is APOBEC1 (“apolipoprotein B mRNA editing enzyme, catalytic polypeptide 1”). Another example is AID (“activation-induced cytidine deaminase”). Under standard Watson-Crick hydrogen bond pairing, a cytosine base hydrogen bonds to a guanine base. When cytidine is converted to uridine (or deoxycytidine is converted to deoxyuridine), the uridine (or the uracil base of uridine) undergoes hydrogen bond pairing with the base adenine. Thus, a conversion of “C” to uridine (“U”) by cytidine deaminase will cause the insertion of “A” instead of a “G” during cellular repair and/or replication processes. Since the adenine “A” pairs with thymine “T”, the cytidine deaminase in coordination with DNA replication causes the conversion of an C G pairing to a T A pairing in the double-stranded DNA molecule.

### CRISPR

CRISPR is a family of DNA sequences (i.e., CRISPR clusters) in bacteria and archaea that represent snippets of prior infections by a virus that have invaded the prokaryote. The snippets of DNA are used by the prokaryotic cell to detect and destroy DNA from subsequent attacks by similar viruses and effectively compose, along with an array of CRISPR-associated proteins (including Cas9 and homologs thereof) and CRISPR-associated RNA, a prokaryotic immune defense system. In nature, CRISPR clusters are transcribed and processed into CRISPR RNA (crRNA). In certain types of CRISPR systems (e.g., type II CRISPR systems), correct processing of pre-crRNA requires a trans-encoded small RNA (tracrRNA), endogenous ribonuclease 3 (rnc) and a Cas9 protein. The tracrRNA serves as a guide for ribonuclease 3-aided processing of pre-crRNA. Subsequently, Cas9/crRNA/tracrRNA endonucleolytically cleaves linear or circular dsDNA target complementary to the RNA. Specifically, the target strand not complementary to crRNA is first cut endonucleolytically, then trimmed 3′-5′ exonucleolytically. In nature, DNA-binding and cleavage typically requires protein and both RNAs. However, single guide RNAs (“sgRNA”, or simply “gRNA”) can be engineered so as to incorporate aspects of both the crRNA and tracrRNA into a single RNA species—the guide RNA. See, e.g., Jinek M., Chylinski K., Fonfara I., Hauer M., Doudna J. A., Charpentier E. Science 337:816-821(2012), the entire contents of which is hereby incorporated by reference. Cas9 recognizes a short motif in the CRISPR repeat sequences (the PAM or protospacer adjacent motif) to help distinguish self versus non-self CRISPR biology, as well as Cas9 nuclease sequences and structures are well known to those of skill in the art (see, e.g., “Complete genome sequence of an M1 strain of Streptococcus pyogenes.” Ferretti et al., J. J., McShan W. M., Ajdic D. J., Savic D. J., Savic G., Lyon K., Primeaux C., Sezate S., Suvorov A. N., Kenton S., Lai H. S., Lin S. P., Qian Y., Jia H. G., Najar F. Z., Ren Q., Zhu H., Song L., White J., Yuan X., Clifton S. W., Roe B. A., McLaughlin R. E., Proc. Natl. Acad. Sci. U.S.A. 98:4658-4663(2001); “CRISPR RNA maturation by trans-encoded small RNA and host factor RNase III.” Deltcheva E., Chylinski K., Sharma C. M., Gonzales K., Chao Y., Pirzada Z. A., Eckert M. R., Vogel J., Charpentier E., Nature 471:602-607(2011); and “A programmable dual-RNA-guided DNA endonuclease in adaptive bacterial immunity.” Jinek M., Chylinski K., Fonfara I., Hauer M., Doudna J. A., Charpentier E. Science 337:816-821(2012), the entire contents of each of which are incorporated herein by reference). Cas9 orthologs have been described in various species, including, but not limited to, S. pyogenes and S. thermophilus. Additional suitable Cas9 nucleases and sequences will be apparent to those of skill in the art based on this disclosure, and such Cas9 nucleases and sequences include Cas9 sequences from the organisms and loci disclosed in Chylinski, Rhun, and Charpentier, “The tracrRNA and Cas9 families of type II CRISPR-Cas immunity systems” (2013) RNA Biology 10:5, 726-737; the entire contents of which are incorporated herein by reference.

### Deaminase

The term “deaminase” or “deaminase domain” refers to a protein or enzyme that catalyzes a deamination reaction. In some embodiments, the deaminase is an adenosine (or adenine) deaminase, which catalyzes the hydrolytic deamination of adenine or adenosine. In some embodiments, the adenosine deaminase catalyzes the hydrolytic deamination of adenine or adenosine in deoxyribonucleic acid (DNA) to inosine. In other embodiments, the deminase is a cytidine (or cytosine) deaminase, which catalyzes the hydrolytic deamination of cytidine or cytosine.

The deaminases provided herein may be from any organism, such as a bacterium. In some embodiments, the deaminase or deaminase domain is a variant of a naturally-occurring deaminase from an organism. In some embodiments, the deaminase or deaminase domain does not occur in nature. For example, in some embodiments, the deaminase or deaminase domain is at least 50%, at least 55%, at least 60%, at least 65%, at least 70%, at least 75% at least 80%, at least 85%, at least 90%, at least 95%, at least 96%, at least 97%, at least 98%, at least 99%, or at least 99.5% identical to a naturally-occurring deaminase.

### DNA Binding Protein

As used herein, the term “DNA binding protein” or “DNA binding protein domain” refers to any protein that localizes to and binds a specific target DNA nucleotide sequence (e.g. a gene locus of a genome). This term embraces RNA-programmable proteins, which associate (e.g. form a complex) with one or more nucleic acid molecules (i.e., which includes, for example, guide RNA in the case of Cas systems) that direct or otherwise program the protein to localize to a specific target nucleotide sequence (e.g., DNA sequence) that is complementary to the one or more nucleic acid molecules (or a portion or region thereof) associated with the protein. Exemplary RNA-programmable proteins are CRISPR-Cas9 proteins, as well as Cas9 equivalents, homologs, orthologs, or paralogs, whether naturally occurring or non-naturally occurring (e.g. engineered or modified), and may include a Cas9 equivalent from any type of CRISPR system (e.g. type II, V, VI), including Cpf1 (a type-V CRISPR-Cas systems), C2c1 (a type V CRISPR-Cas system), C2c2 (a type VI CRISPR-Cas system), C2c3 (a type V CRISPR-Cas system), dCas9, GeoCas9, CjCas9, Cas12a, Cas12b, Cas12c, Cas12d, Cas12g, Cas12h, Cas12i, Cas13d, Cas14, Argonaute, and nCas9. Further Cas-equivalents are described in Makarova et al., “C2c2 is a single-component programmable RNA-guided RNA-targeting CRISPR effector,” Science 2016; 353(6299), the contents of which are incorporated herein by reference.

### DNA Editing Efficiency

The term “DNA editing efficiency,” as used herein, refers to the number or proportion of intended base pairs that are edited. For example, if a base editor edits 10% of the base pairs that it is intended to target (e.g., within a cell or within a population of cells), then the base editor can be described as being 10% efficient. Some aspects of editing efficiency embrace the modification (e.g. deamination) of a specific nucleotide within DNA, without generating a large number or percentage of insertions or deletions (i.e., indels). It is generally accepted that editing while generating less than 5% indels (as measured over total target nucleotide substrates) is high editing efficiency. The generation of more than 20% indels is generally accepted as poor or low editing efficiency. Indel formation may be measured by techniques known in the art, including high-throughput screening of sequencing reads.

### Downstream

As used herein, the terms “upstream” and “downstream” are terms of relativety that define the linear position of at least two elements located in a nucleic acid molecule (whether single or double-stranded) that is orientated in a 5′-to-3′ direction. In particular, a first element is upstream of a second element in a nucleic acid molecule where the first element is positioned somewhere that is 5′ to the second element. For example, a SNP is upstream of a Cas9-induced nick site if the SNP is on the 5′ side of the nick site. Conversely, a first element is downstream of a second element in a nucleic acid molecule where the first element is positioned somewhere that is 3′ to the second element. For example, a SNP is downstream of a Cas9-induced nick site if the SNP is on the 3′ side of the nick site. The nucleic acid molecule can be a DNA (double or single stranded). RNA (double or single stranded), or a hybrid of DNA and RNA. The analysis is the same for single strand nucleic acid molecule and a double strand molecule since the terms upstream and downstream are in reference to only a single strand of a nucleic acid molecule, except that one needs to select which strand of the double stranded molecule is being considered. Often, the strand of a double stranded DNA which can be used to determine the positional relativity of at least two elements is the “sense” or “coding” strand. In genetics, a “sense” strand is the segment within double-stranded DNA that runs from 5′ to 3′, and which is complementary to the antisense strand of DNA, or template strand, which runs from 3′ to 5′. Thus, as an example, a SNP nucleobase is “downstream” of a promoter sequence in a genomic DNA (which is double-stranded) if the SNP nucleobase is on the 3′ side of the promoter on the sense or coding strand.

### Effective Amount

The term “effective amount,” as used herein, refers to an amount of a biologically active agent that is sufficient to elicit a desired biological response. For example, in some embodiments, an effective amount of a base editor may refer to the amount of the editor that is sufficient to edit a target site nucleotide sequence, e.g., a genome. In some embodiments, an effective amount of a base editor provided herein, e.g., of a fusion protein comprising a nickase Cas9 domain and a guide RNA may refer to the amount of the fusion protein that is sufficient to induce editing of a target site specifically bound and edited by the fusion protein. As will be appreciated by the skilled artisan, the effective amount of an agent, e.g., a fusion protein, a nuclease, a hybrid protein, a protein dimer, a complex of a protein (or protein dimer) and a polynucleotide, or a polynucleotide, may vary depending on various factors as, for example, on the desired biological response, e.g., on the specific allele, genome, or target site to be edited, on the cell or tissue being targeted, and on the agent being used.

### Functional Equivalent

The term “functional equivalent” refers to a second biomolecule that is equivalent in function, but not necessarily equivalent in structure to a first biomolecule. For example, a “Cas9 equivalent” refers to a protein that has the same or substantially the same functions as Cas9, but not necessarily the same amino acid sequence. In the context of the disclosure, the specification refers throughout to “a protein X, or a functional equivalent thereof” In this context, a “functional equivalent” of protein X embraces any homolog, paralog, fragment, naturally occurring, engineered, circular permutant, mutated, or synthetic version of protein X which bears an equivalent function.

### Fusion Protein

The term “fusion protein” as used herein refers to a hybrid polypeptide which comprises protein domains from at least two different proteins. One protein may be located at the amino-terminal (N-terminal) portion of the fusion protein or at the carboxy-terminal (C-terminal) protein thus forming an “amino-terminal fusion protein” or a “carboxy-terminal fusion protein,” respectively. A protein may comprise different domains, for example, a nucleic acid binding domain (e.g., the gRNA binding domain of Cas9 that directs the binding of the protein to a target site) and a nucleic acid cleavage domain or a catalytic domain of a nucleic-acid editing protein. Another example includes a Cas9 or equivalent thereof fused to an adenosine deaminae. Any of the proteins provided herein may be produced by any method known in the art. For example, the proteins provided herein may be produced via recombinant protein expression and purification, which is especially suited for fusion proteins comprising a peptide linker. Methods for recombinant protein expression and purification are well known, and include those described by Green and Sambrook, Molecular Cloning: A Laboratory Manual (4^{th} ed., Cold Spring Harbor Laboratory Press, Cold Spring Harbor, N.Y. (2012)), the entire contents of which are incorporated herein by reference.

### Guide Nucleic Acid

The term “guide nucleic acid” or “napDNAbp-programming nucleic acid molecule” or equivalently “guide sequence” refers the one or more nucleic acid molecules which associate with and direct or otherwise program a napDNAbp protein to localize to a specific target nucleotide sequence (e.g., a gene locus of a genome) that is complementary to the one or more nucleic acid molecules (or a portion or region thereof) associated with the protein, thereby causing the napDNAbp protein to bind to the nucleotide sequence at the specific target site. A non-limiting example is a guide RNA of a Cas protein of a CRISPR-Cas genome editing system.

Guide RNA is a particular type of guide nucleic acid which is mostly commonly associated with a Cas protein of a CRISPR-Cas9 and which associates with Cas9, directing the Cas9 protein to a specific sequence in a DNA molecule that includes complementarity to protospace sequence of the guide RNA. As used herein, a “guide RNA” refers to a synthetic fusion of the endogenous bacterial crRNA and tracrRNA that provides both targeting specificity and scaffolding and/or binding ability for Cas9 nuclease to a target DNA. This synthetic fusion does not exist in nature and is also commonly referred to as an sgRNA. However, this term also embraces the equivalent guide nucleic acid molecules that associate with Cas9 equivalents, homologs, orthologs, or paralogs, whether naturally occurring or non-naturally occurring (e.g., engineered or recombinant), and which otherwise program the Cas9 equivalent to localize to a specific target nucleotide sequence. The Cas9 equivalents may include other napDNAbp from any type of CRISPR system (e.g., type II, V, VI), including Cpf1 (a type-V CRISPR-Cas systems), C2c1 (a type V CRISPR-Cas system), C2c2 (a type VI CRISPR-Cas system) and C2c3 (a type V CRISPR-Cas system). Further Cas-equivalents are described in Makarova et al., “C2c2 is a single-component programmable RNA-guided RNA-targeting CRISPR effector,” Science 2016; 353(6299), the contents of which are incorporated herein by reference. Exemplary sequences are and structures of guide RNAs are provided herein. In addition, methods for designing appropriate guide RNA sequences are provided herein.

Guide RNA (“gRNA”)

As used herein, the term “guide RNA” is a particular type of guide nucleic acid which is mostly commonly associated with a Cas protein of a CRISPR-Cas9 and which associates with Cas9, directing the Cas9 protein to a specific sequence in a DNA molecule that includes complementarity to protospace sequence of the guide RNA. However, this term also embraces the equivalent guide nucleic acid molecules that associate with Cas9 equivalents, homologs, orthologs, or paralogs, whether naturally occurring or non-naturally occurring (e.g., engineered or recombinant), and which otherwise program the Cas9 equivalent to localize to a specific target nucleotide sequence. The Cas9 equivalents may include other napDNAbp from any type of CRISPR system (e.g., type II, V, VI), including Cpf1 (a type-V CRISPR-Cas systems), C2c1 (a type V CRISPR-Cas system), C2c2 (a type VI CRISPR-Cas system) and C2c3 (a type V CRISPR-Cas system). Further Cas-equivalents are described in Makarova et al., “C2c2 is a single-component programmable RNA-guided RNA-targeting CRISPR effector,” Science 2016; 353(6299), the contents of which are incorporated herein by reference. Exemplary sequences are and structures of guide RNAs are provided herein.

Guide RNAs may comprise various structural elements that include, but are not limited to (a) a spacer sequence—the sequence in the guide RNA (having ˜20 nts in length) which binds to a complementary strand of the target DNA (and has the same sequence as the protospacer of the DNA) and (b) a gRNA core (or gRNA scaffold or backbone sequence)—refers to the sequence within the gRNA that is responsible for Cas9 binding, it does not include the ˜20 bp spacer sequence that is used to guide Cas9 to target DNA.

### Guide RNA Target Sequence

As used herein, the “guide RNA target sequence” refers to the ˜20 nucleotides that are complementary to the protospacer sequence in the PAM strand. The target sequence is the sequence that anneals to or is targeted by the spacer sequence of the guide RNA. The spacer sequence of the guide RNA and the protospacer have the same sequence (except the spacer sequence is RNA and the protospacer is DNA).

### Guide RNA Scaffold Sequence

As used herein, the “guide RNA scaffold sequence” refers to the sequence within the gRNA that is responsible for Cas9 binding, it does not include the 20 bp spacer/targeting sequence that is used to guide Cas9 to target DNA.

### Host Cell

The term “host cell,” as used herein, refers to a cell that can host, replicate, and transfer a phage vector useful for a continuous evolution process as provided herein. In embodiments where the vector is a viral vector, a suitable host cell is a cell that may be infected by the viral vector, can replicate it, and can package it into viral particles that can infect fresh host cells. A cell can host a viral vector if it supports expression of genes of viral vector, replication of the viral genome, and/or the generation of viral particles. One criterion to determine whether a cell is a suitable host cell for a given viral vector is to determine whether the cell can support the viral life cycle of a wild-type viral genome that the viral vector is derived from. For example, if the viral vector is a modified M13 phage genome, as provided in some embodiments described herein, then a suitable host cell would be any cell that can support the wild-type M13 phage life cycle. Suitable host cells for viral vectors useful in continuous evolution processes are well known to those of skill in the art, and the disclosure is not limited in this respect. In some embodiments, the viral vector is a phage and the host cell is a bacterial cell. In some embodiments, the host cell is an E. coli cell. Suitable E. coli host strains will be apparent to those of skill in the art, and include, but are not limited to, New England Biolabs (NEB) Turbo, Top10F′, DH12S, ER2738, ER2267, and XL1-Blue MRF′. These strain names are art recognized and the genotype of these strains has been well characterized. It should be understood that the above strains are exemplary only and that the invention is not limited in this respect. The term “fresh,” as used herein interchangeably with the terms “non-infected” or “uninfected” in the context of host cells, refers to a host cell that has not been infected by a viral vector comprising a gene of interest as used in a continuous evolution process provided herein. A fresh host cell can, however, have been infected by a viral vector unrelated to the vector to be evolved or by a vector of the same or a similar type but not carrying the gene of interest.

In some embodiments, the host cell is a prokaryotic cell, for example, a bacterial cell. In some embodiments, the host cell is an E. coli cell. In some embodiments, the host cell is a eukaryotic cell, for example, a yeast cell, an insect cell, or a mammalian cell. The type of host cell, will, of course, depend on the viral vector employed, and suitable host cell/viral vector combinations will be readily apparent to those of skill in the art.

### Inteins and Split-Inteins

As used herein, the term “intein” refers to auto-processing polypeptide domains found in organisms from all domains of life. An intein (intervening protein) carries out a unique auto-processing event known as protein splicing in which it excises itself out from a larger precursor polypeptide through the cleavage of two peptide bonds and, in the process, ligates the flanking extein (external protein) sequences through the formation of a new peptide bond. This rearrangement occurs post-translationally (or possibly co-translationally), as intein genes are found embedded in frame within other protein-coding genes. Furthermore, intein-mediated protein splicing is spontaneous; it requires no external factor or energy source, only the folding of the intein domain. This process is also known as cis-protein splicing, as opposed to the natural process of trans-protein splicing with “split inteins.”

Split inteins are a sub-category of inteins. Unlike the more common contiguous inteins, split inteins are transcribed and translated as two separate polypeptides, the N-intein and C-intein, each fused to one extein. Upon translation, the intein fragments spontaneously and non-covalently assemble into the canonical intein structure to carry out protein splicing in trans.

Inteins and split inteins are the protein equivalent of the self-splicing RNA introns (see Perler et al., Nucleic Acids Res. 22:1125-1127 (1994)), which catalyze their own excision from a precursor protein with the concomitant fusion of the flanking protein sequences, known as exteins (reviewed in Perler et al., Curr. Opin. Chem. Biol. 1:292-299 (1997); Perler, F. B. Cell 92(1):1-4 (1998); Xu et al., EMBO J. 15(19):5146-5153 (1996)).

As used herein, the term “protein splicing” refers to a process in which an interior region of a precursor protein (an intein) is excised and the flanking regions of the protein (exteins) are ligated to form the mature protein. This natural process has been observed in numerous proteins from both prokaryotes and eukaryotes (Perler, F. B., Xu, M. Q., Paulus, H. Current Opinion in Chemical Biology 1997, 1, 292-299; Perler, F. B. Nucleic Acids Research 1999, 27, 346-347). The intein unit contains the necessary components needed to catalyze protein splicing and often contains an endonuclease domain that participates in intein mobility (Perler, F. B., Davis, E. O., Dean, G. E., Gimble, F. S., Jack, W. E., Neff, N., Noren, C. J., Thomer, J., Belfort, M. Nucleic Acids Research 1994, 22, 1127-1127). The resulting proteins are linked, however, not expressed as separate proteins. Protein splicing may also be conducted in trans with split inteins expressed on separate polypeptides spontaneously combine to form a single intein which then undergoes the protein splicing process to join to separate proteins.

The elucidation of the mechanism of protein splicing has led to a number of intein-based applications (Comb, et al., U.S. Pat. No. 5,496,714; Comb, et al., U.S. Pat. No. 5,834,247; Camarero and Muir, J. Amer. Chem. Soc., 121:5597-5598 (1999); Chong, et al., Gene, 192:271-281 (1997), Chong, et al., Nucleic Acids Res., 26:5109-5115 (1998); Chong, et al., J. Biol. Chem., 273:10567-10577 (1998); Cotton, et al. J. Am. Chem. Soc., 121:1100-1101 (1999); Evans, et al., J. Biol. Chem., 274:18359-18363 (1999); Evans, et al., J. Biol. Chem., 274:3923-3926 (1999); Evans, et al., Protein Sci., 7:2256-2264 (1998); Evans, et al., J. Biol. Chem., 275:9091-9094 (2000); Iwai and Pluckthun, FEBS Lett. 459:166-172 (1999); Mathys, et al., Gene, 231:1-13 (1999); Mills, et al., Proc. Natl. Acad. Sci. USA 95:3543-3548 (1998); Muir, et al., Proc. Natl. Acad. Sci. USA 95:6705-6710 (1998); Otomo, et al., Biochemistry 38:16040-16044 (1999); Otomo, et al., J. Biolmol. NMR 14:105-114 (1999); Scott, et al., Proc. Natl. Acad. Sci. USA 96:13638-13643 (1999); Severinov and Muir, J. Biol. Chem., 273:16205-16209 (1998); Shingledecker, et al., Gene, 207:187-195 (1998); Southworth, et al., EMBO J. 17:918-926 (1998); Southworth, et al., Biotechniques, 27:110-120 (1999); Wood, et al., Nat. Biotechnol., 17:889-892 (1999); Wu, et al., Proc. Natl. Acad. Sci. USA 95:9226-9231 (1998a); Wu, et al., Biochim Biophys Acta 1387:422-432 (1998b); Xu, et al., Proc. Natl. Acad. Sci. USA 96:388-393 (1999); Yamazaki, et al., J. Am. Chem. Soc., 120:5591-5592 (1998)). Each reference is incorporated herein by reference.

### Ligand-Dependent Intein

The term “ligand-dependent intein,” as used herein refers to an intein that comprises a ligand-binding domain. Typically, the ligand-binding domain is inserted into the amino acid sequence of the intein, resulting in a structure intein (N)-ligand-binding domain-intein (C). Typically, ligand-dependent inteins exhibit no or only minimal protein splicing activity in the absence of an appropriate ligand, and a marked increase of protein splicing activity in the presence of the ligand. In some embodiments, the ligand-dependent intein does not exhibit observable splicing activity in the absence of ligand but does exhibit splicing activity in the presence of the ligand. In some embodiments, the ligand-dependent intein exhibits an observable protein splicing activity in the absence of the ligand, and a protein splicing activity in the presence of an appropriate ligand that is at least 5 times, at least 10 times, at least 50 times, at least 100 times, at least 150 times, at least 200 times, at least 250 times, at least 500 times, at least 1000 times, at least 1500 times, at least 2000 times, at least 2500 times, at least 5000 times, at least 10000 times, at least 20000 times, at least 25000 times, at least 50000 times, at least 100000 times, at least 500000 times, or at least 1000000 times greater than the activity observed in the absence of the ligand. In some embodiments, the increase in activity is dose dependent over at least 1 order of magnitude, at least 2 orders of magnitude, at least 3 orders of magnitude, at least 4 orders of magnitude, or at least 5 orders of magnitude, allowing for fine-tuning of intein activity by adjusting the concentration of the ligand. Suitable ligand-dependent inteins are known in the art, and in include those provided below and those described in published U.S. Patent Application U.S. 2014/0065711 A1; Mootz et al., “Protein splicing triggered by a small molecule.” J. Am. Chem. Soc. 2002; 124, 9044-9045; Mootz et al., “Conditional protein splicing: a new tool to control protein structure and function in vitro and in vivo.” J. Am. Chem. Soc. 2003; 125, 10561-10569; Buskirk et al., Proc. Natl. Acad. Sci. USA. 2004; 101, 10505-10510); Skretas & Wood, “Regulation of protein activity with small-molecule-controlled inteins.” Protein Sci. 2005; 14, 523-532; Schwartz, et al., “Post-translational enzyme activation in an animal via optimized conditional protein splicing.” Nat. Chem. Biol. 2007; 3, 50-54; Peck et al., Chem. Biol. 2011; 18 (5), 619-630; the entire contents of each are hereby incorporated by reference. Exemplary sequences are as follows:

| NAME | SEQUENCE OF LIGAND-DEPENDENT INTEIN |
|---|---|
| 2-4 INTEIN: | CLAEGTRIFDPVTGTTHRIEDVVDGRKPIHVVAAAKDGTLLARPVVSWFDQGTRDVIGLRIAGGAIV |
| | WATPDHKVLTEYGWRAAGELRKGDRVAGPGGSGNSLALSLTADQMVSALLDAEPPILYSEYDPTSPF |
| | SEASMMGLLTNLADRELVHMINWAKRVPGFVDLTLHDQAHLLECAWLEILMIGLVWRSMEHPGKLLF |
| | APNLLLDRNQGKCVEGMVEIFDMLLATSSRFRMMNLQGEEFVCLKSIILLNSGVYTFLSSTLKSLEE |
| | KDHIHRALDKITDTLIHLMAKAGLTLQQQHQRLAQLLLILSHIRHMSNKGMEHLYSMKYKNVVPLYD |
| | LLLEMLDAHRLHAGGSGASRVQAFADALDDKFLHDMLAEELRYSVIREVLPTRRARTFDLEVEELHT |
| | LVAEGVVVHNC (SEQ ID NO: 164) |
| 3-2 INTEIN | CLAEGTRIFDPVTGTTHRIEDVVDGRKPIHVVAVAKDGTLLARPVVSWFDQGTRDVIGLRIAGGAIV |
| | WATPDHKVLTEYGWRAAGELRKGDRVAGPGGSGNSLALSLTADQMVSALLDAEPPILYSEYDPTSPF |
| | SEASMMGLLTNLADRELVHMINWAKRVPGFVDLTLHDQAHLLECAWLEILMIGLVWRSMEHPGKLLF |
| | APNLLLDRNQGKCVEGMVEIFDMLLATSSRFRMMNLQGEEFVCLKSIILLNSGVYTFLSSTLKSLEE |
| | KDHIHRALDKITDTLIHLMAKAGLTLQQQHQRLAQLLLILSHIRHMSNKGMEHLYSMKYTNVVPLYD |
| | LLLEMLDAHRLHAGGSGASRVQAFADALDDKFLHDMLAEELRYSVIREVLPTRRARTFDLEVEELHT |
| | LVAEGVVVHNC (SEQ ID NO: 165) |
| 30R3-1 INTEIN | CLAEGTRIFDPVTGTTHRIEDVVDGRKPIHVVAAAKDGTLLARPVVSWFDQGTRDVIGLRIAGGATV |
| | WATPDHKVLTEYGWRAAGELRKGDRVAGPGGSGNSLALSLTADQMVSALLDAEPPIPYSEYDPTSPF |
| | SEASMMGLLTNLADRELVHMINWAKRVPGFVDLTLHDQAHLLECAWLEILMIGLVWRSMEHPGKLLF |
| | APNLLLDRNQGKCVEGMVEIFDMLLATSSRFRMMNLQGEEFVCLKSIILLNSGVYTFLSSTLKSLEE |
| | KDHIHRALDKITDTLIHLMAKAGLTLQQQHQRLAQLLLILSHIRHMSNKGMEHLYSMKYKNVVPLYD |
| | LLLEMLDAHRLHAGGSGASRVQAFADALDDKFLHDMLAEGLRYSVIREVLPTRRARTFDLEVEELHT |
| | LVAEGVVVHNC (SEQ ID NO: 166) |
| 30R3-2 INTEIN | CLAEGTRIFDPVTGTTHRIEDVVDGRKPIHVVAAAKDGTLLARPVVSWFDQGTRDVIGLRIAGGATV |
| | WATPDHKVLTEYGWRAAGELRKGDRVAGPGGSGNSLALSLTADQMVSALLDAEPPILYSEYDPTSPF |
| | SEASMMGLLTNLADRELVHMINWAKRVPGFVDLTLHDQAHLLECAWLEILMIGLVWRSMEHPGKLLF |
| | APNLLLDRNQGKCVEGMVEIFDMLLATSSRFRMMNLQGEEFVCLKSIILLNSGVYTFLSSTLKSLEE |
| | KDHIHRALDKITDTLIHLMAKAGLTLQQQHQRLAQLLLILSHIRHMSNKGMEHLYSMKYKNVVPLYD |
| | LLLEMLDAHRLHAGGSGASRVQAFADALDDKFLHDMLAEELRYSVIREVLPTRRARTFDLEVEELHT |
| | LVAEGVVVHNC (SEQ ID NO: 167) |
| 30R3-3 INTEIN | CLAEGTRIFDPVTGTTHRIEDVVDGRKPIHVVAAAKDGTLLARPVVSWFDQGTRDVIGLRIAGGATV |
| | WATPDHKVLTEYGWRAAGELRKGDRVAGPGGSGNSLALSLTADQMVSALLDAEPPIPYSEYDPTSPF |
| | SEASMMGLLTNLADRELVHMINWAKRVPGFVDLTLHDQAHLLECAWLEILMIGLVWRSMEHPGKLLF |
| | APNLLLDRNQGKCVEGMVEIFDMLLATSSRFRMMNLQGEEFVCLKSIILLNSGVYTFLSSTLKSLEE |
| | KDHIHRALDKITDTLIHLMAKAGLTLQQQHQRLAQLLLILSHIRHMSNKGMEHLYSMKYKNVVPLYD |
| | LLLEMLDAHRLHAGGSGASRVQAFADALDDKFLHDMLAEELRYSVIREVLPTRRARTFDLEVEELHT |
| | LVAEGVVVHNC (SEQ ID NO: 168) |
| 37R3-1 INTEIN | CLAEGTRIFDPVTGTTHRIEDVVDGRKPIHVVAAAKDGTLLARPVVSWFDQGTRDVIGLRIAGGATV |
| | WATPDHKVLTEYGWRAAGELRKGDRVAGPGGSGNSLALSLTADQMVSALLDAEPPILYSEYNPTSPF |
| | SEASMMGLLTNLADRELVHMINWAKRVPGFVDLTLHDQAHLLERAWLEILMIGLVWRSMEHPGKLLF |
| | APNLLLDRNQGKCVEGMVEIFDMLLATSSRFRMMNLQGEEFVCLKSIILLNSGVYTFLSSTLKSLEE |
| | KDHIHRALDKITDTLIHLMAKAGLTLQQQHQRLAQLLLILSHIRHMSNKGMEHLYSMKYKNVVPLYD |
| | LLLEMLDAHRLHAGGSGASRVQAFADALDDKFLHDMLAEGLRYSVIREVLPTRRARTFDLEVEELHT |
| | LVAEGVVVHNC ((SEQ ID NO: 169) |
| 37R3-2 INTEIN | CLAEGTRIFDPVTGTTHRIEDVVDGRKPIHVVAAAKDGTLLARPVVSWFDQGTRDVIGLRIAGGAIV |
| | WATPDHKVLTEYGWRAAGELRKGDRVAGPGGSGNSLALSLTADQMVSALLDAEPPILYSEYDPTSPF |
| | SEASMMGLLTNLADRELVHMINWAKRVPGFVDLTLHDQAHLLERAWLEILMIGLVWRSMEHPGKLLF |
| | APNLLLDRNQGKCVEGMVEIFDMLLATSSRFRMMNLQGEEFVCLKSIILLNSGVYTFLSSTLKSLEE |
| | KDHIHRALDKITDTLIHLMAKAGLTLQQQHQRLAQLLLILSHIRHMSNKGMEHLYSMKYKNVVPLYD |
| | LLLEMLDAHRLHAGGSGASRVQAFADALDDKFLHDMLAEGLRYSVIREVLPTRRARTFDLEVEELHT |
| | LVAEGVVVHNC (SEQ ID NO: 170) |
| 37R3-3 INTEIN | CLAEGTRIFDPVTGTTHRIEDVVDGRKPIHVVAVAKDGTLLARPVVSWFDQGTRDVIGLRIAGGATV |
| | WATPDHKVLTEYGWRAAGELRKGDRVAGPGGSGNSLALSLTADQMVSALLDAEPPILYSEYDPTSPF |
| | SEASMMGLLTNLADRELVHMINWAKRVPGFVDLTLHDQAHLLERAWLEILMIGLVWRSMEHPGKLLF |
| | APNLLLDRNQGKCVEGMVEIFDMLLATSSRFRMMNLQGEEFVCLKSIILLNSGVYTFLSSTLKSLEE |
| | KDHIHRALDKITDTLIHLMAKAGLTLQQQHQRLAQLLLILSHIRHMSNKGMEHLYSMKYKNVVPLYD |
| | LLLEMLDAHRLHAGGSGASRVQAFADALDDKFLHDMLAEELRYSVIREVLPTRRARTFDLEVEELHT |
| | LVAEGVVVHNC (SEQ ID NO: 171) |

### Linker

The term “linker,” as used herein, refers to a chemical group or a molecule linking two molecules or domains, e.g. dCas9 and a deaminase. Typically, the linker is positioned between, or flanked by, two groups, molecules, or other domains and connected to each one via a covalent bond, thus connecting the two. In some embodiments, the linker is an amino acid or a plurality of amino acids (e.g. a peptide or protein). In some embodiments, the linker is an organic molecule, group, polymer, or chemical domain. Chemical groups include, but are not limited to, disulfide, hydrazone, and azide domains. In some embodiments, the linker is 5-100 amino acids in length, for example, 5, 6, 7, 8, 9, 10, 11, 12, 13, 14, 15, 16, 17, 18, 19, 20, 21, 22, 23, 24, 25, 26, 27, 28, 29, 30, 30-35, 35-40, 40-45, 45-50, 50-60, 60-70, 70-80, 80-90, 90-100, 100-150, or 150-200 amino acids in length. Longer or shorter linkers are also contemplated. In some embodiments, the linker is an XTEN linker. In some embodiments, the linker is a 32-amino acid linker. In other embodiments, the linker is a 30-, 31-, 33- or 34-amino acid linker.

### Mutation

The term “mutation,” as used herein, refers to a substitution of a residue within a sequence, e.g. a nucleic acid or amino acid sequence, with another residue; a deletion or insertion of one or more residues within a sequence; or a substitution of a residue within a sequence of a genome in a subject to be corrected. Mutations are typically described herein by identifying the original residue followed by the position of the residue within the sequence and by the identity of the newly substituted residue. Various methods for making the amino acid substitutions (mutations) provided herein are well known in the art, and are provided by, for example, Green and Sambrook, Molecular Cloning: A Laboratory Manual (4^{th} ed., Cold Spring Harbor Laboratory Press, Cold Spring Harbor, N.Y. (2012)). Mutations can include a variety of categories, such as single base polymorphisms, microduplication regions, indel, and inversions, and is not meant to be limiting in any way. Mutations can include “loss-of-function” mutations which are mutations that reduce or abolish a protein activity. Most loss-of-function mutations are recessive, because in a heterozygote the second chromosome copy carries an unmutated version of the gene coding for a fully functional protein whose presence compensates for the effect of the mutation. There are some exceptions where a loss-of-function mutation is dominant, one example being haploinsufficiency, where the organism is unable to tolerate the approximately 50% reduction in protein activity suffered by the heterozygote. This is the explanation for a few genetic diseases in humans, including Marfan syndrome, which results from a mutation in the gene for the connective tissue protein called fibrillin. Mutations also embrace “gain-of-function” mutations, which is one which confers an abnormal activity on a protein or cell that is otherwise not present in a normal condition. Many gain-of-function mutations are in regulatory sequences rather than in coding regions, and can therefore have a number of consequences. For example, a mutation might lead to one or more genes being expressed in the wrong tissues, these tissues gaining functions that they normally lack. Alternatively the mutation could lead to overexpression of one or more genes involved in control of the cell cycle, thus leading to uncontrolled cell division and hence to cancer. Because of their nature, gain-of-function mutations are usually dominant.

### On-Target Editing

The term “on-target editing,” as used herein, refers to the introduction of intended modifications (e.g., deaminations) to nucleotides (e.g., adenine) in a target sequence, such as using the base editors described herein. The term “off-target DNA editing,” as used herein, refers to the introduction of unintended modifications (e.g. deaminations) to nucleotides (e.g. adenine) in a sequence outside the canonical base editor binding window (i.e., from one protospacer position to another, typically 2 to 8 nucleotides long). Off-target DNA editing can result from weak or non-specific binding of the gRNA sequence to the target sequence.

### Off-Target Editing

The term “off-target editing” or “Cas9-dependent off-target editing” refers to the introduction of unintended modifications that result from weak or non-specific binding of a napDNAbp-gRNA complex (e.g., a complex between a gRNA and the base editor's napDNAbp domain) to nucleic acid sites that have fairly high (e.g. more than 60%, or having fewer than 6 mismatches relative to) sequence identity to a target sequence. In contrast, the term “Cas9-independent off-target editing” refers to the introduction of unintended modifications that result from weak associations of a base editor (e.g., the nucleotide modification domain) to nucleic acid sites that do not have high sequence identity (about 60% or less, or having 6-8 or more mismatches relative to) to a target sequence. Because these associations occur independent of any hybridization between the Cas9-gRNA complex and the relevant nucleic acid site, they are referred to as “Cas9-independent.”

The term “off-target editing frequency,” as used herein, refers to the number or proportion of unintended base pairs that are edited. On-target and off-target editing frequencies may be measured by the methods and assays described herein, further in view of techniques known in the art, including high-throughput sequencing reads. As used herein, high-throughput sequencing involves the hybridization of nucleic acid primers (e.g., DNA primers) with complementarity to nucleic acid (e.g., DNA) regions just upstream or downstream of the target sequence or off-target sequence of interest. Because the DNA target sequence and the Cas9-independent off-target sequences are known apriori in the methods disclosed herein, nucleic acid primers with sufficient complementarity to regions upstream or downstream of the target sequence and Cas9-independent off-target sequences of interest may be designed using techniques known in the art, such as the PhusionU PCR kit (Life Technologies), Phusion HS II kit (Life Technologies), and Illumina MiSeq kit. Since many of the Cas9-dependent off-target sites have high sequence identity to the target site of interest, nucleic acid primers with sufficient complementarity to regions upstream or downstream of the Cas9-dependent off-target site may likewise be designed using techniques and kits known in the art. These kits make use of polymerase chain reaction (PCR) amplification, which produces amplicons as intermediate products. The target and off-target sequences may comprise genomic loci that further comprise protospacers and PAMs. Accordingly, the term “amplicons,” as used herein, may refer to nucleic acid molecules that constitute the aggregates of genomic loci, protospacers and PAMs. High-throughput sequencing techniques used herein may further include Sanger sequencing and/or whole genome sequencing (WGS).

napDNAbp

The term “napDNAb” which stand for “nucleic acid programmable DNA binding protein” refers to any protein that may associate (e.g., form a complex) with one or more nucleic acid molecules (i.e., which may broadly be referred to as a “napDNAbp-programming nucleic acid molecule” and includes, for example, guide RNA in the case of Cas systems) which direct or otherwise program the protein to localize to a specific target nucleotide sequence (e.g., a gene locus of a genome) that is complementary to the one or more nucleic acid molecules (or a portion or region thereof) associated with the protein, thereby causing the protein to bind to the nucleotide sequence at the specific target site. This term napDNAbp embraces CRISPR-Cas9 proteins, as well as Cas9 equivalents, homologs, orthologs, or paralogs, whether naturally occurring or non-naturally occurring (e.g., engineered or modified), and may include a Cas9 equivalent from any type of CRISPR system (e.g., type II, V, VI), including Cpf1 (a type-V CRISPR-Cas systems), C2c1 (a type V CRISPR-Cas system), C2c2 (a type VI CRISPR-Cas system), C2c3 (a type V CRISPR-Cas system), dCas9, GeoCas9, CjCas9, Cas12a, Cas12b, Cas12c, Cas12d, Cas12g, Cas12h, Cas12i, Cas13d, Cas14, Argonaute, and nCas9. Further Cas-equivalents are described in Makarova et al., “C2c2 is a single-component programmable RNA-guided RNA-targeting CRISPR effector,” Science 2016; 353 (6299), the contents of which are incorporated herein by reference. However, the nucleic acid programmable DNA binding protein (napDNAbp) that may be used in connection with this invention are not limited to CRISPR-Cas systems. The invention embraces any such programmable protein, such as the Argonaute protein from Natronobacterium gregoryi (NgAgo) which may also be used for DNA-guided genome editing. NgAgo-guide DNA system does not require a PAM sequence or guide RNA molecules, which means genome editing can be performed simply by the expression of generic NgAgo protein and introduction of synthetic oligonucleotides on any genomic sequence. See Gao et al., DNA-guided genome editing using the Natronobacterium gregoryi Argonaute. Nature Biotechnology 2016; 34(7):768-73, which is incorporated herein by reference.

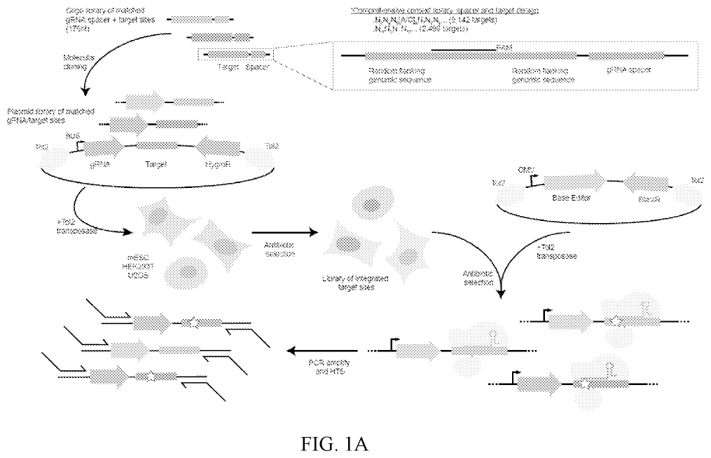

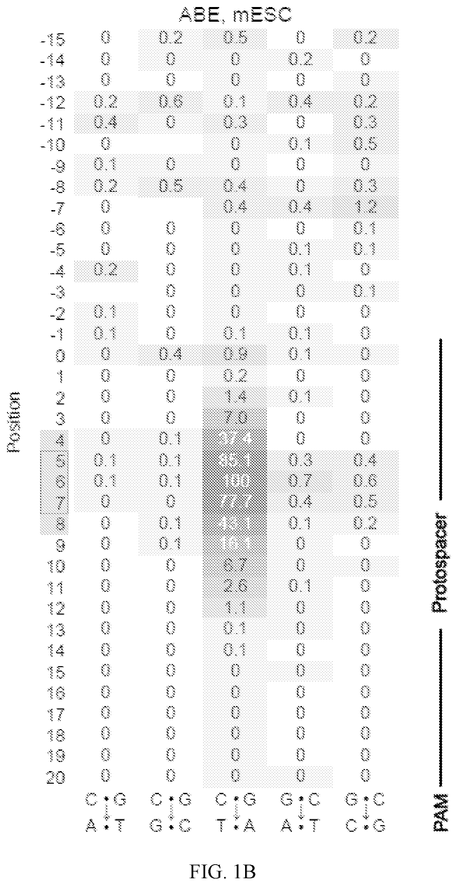

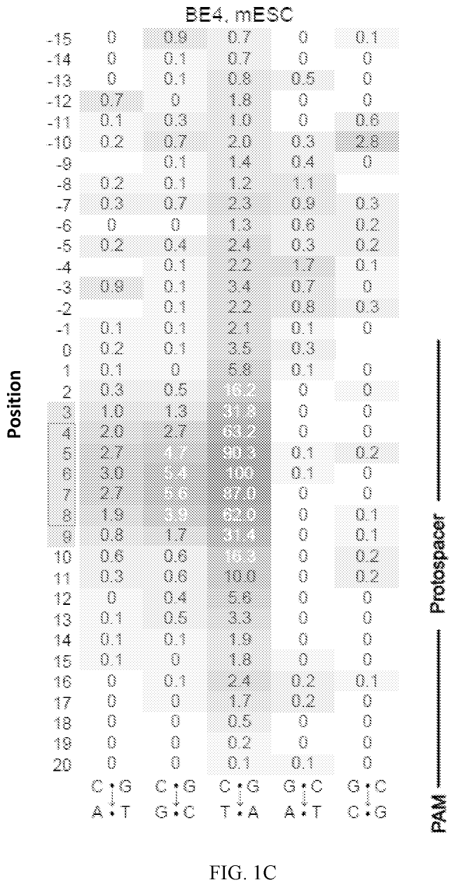

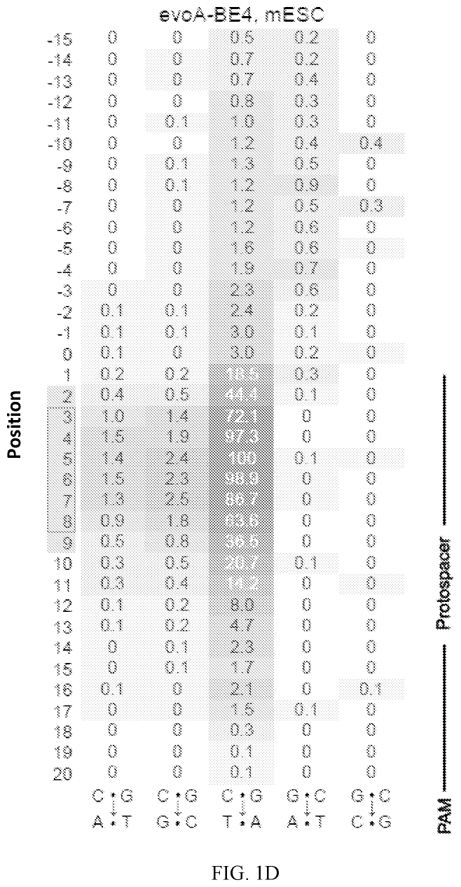

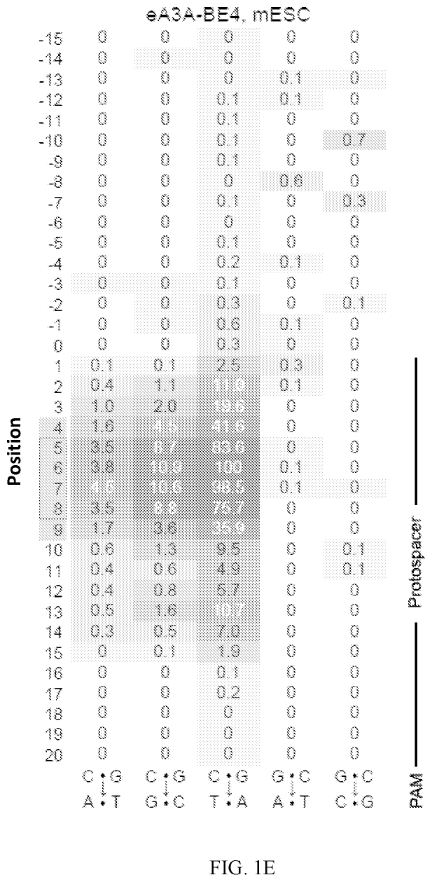

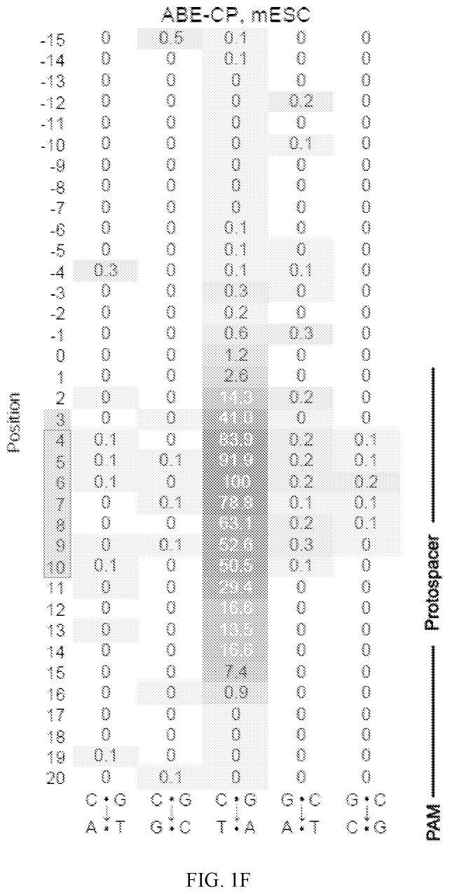

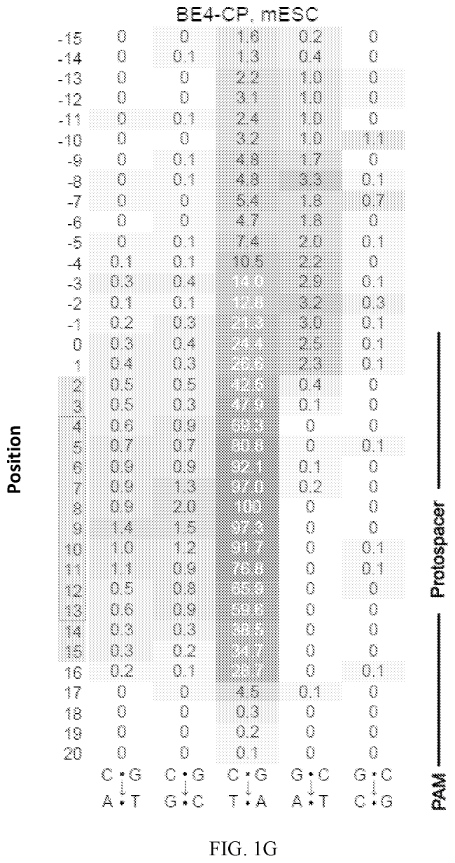

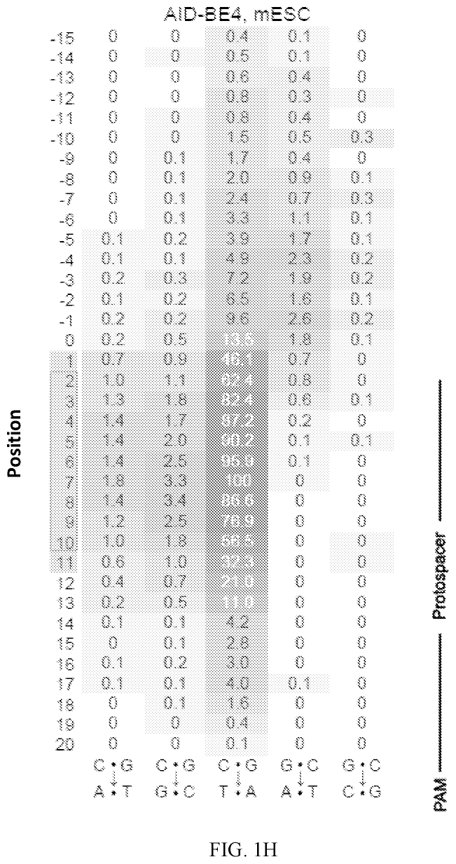

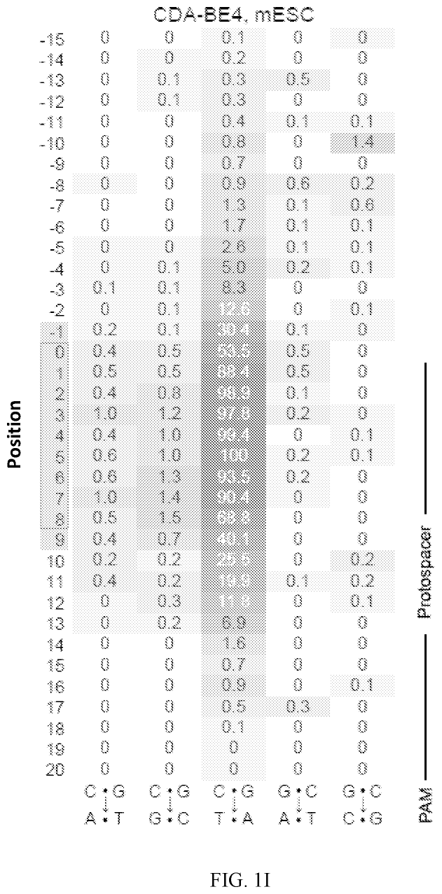

In some embodiments, the napDNAbp is a RNA-programmable nuclease, when in a complex with an RNA, may be referred to as a nuclease:RNA complex. Typically, the bound RNA(s) is referred to as a guide RNA (gRNA). gRNAs can exist as a complex of two or more RNAs, or as a single RNA molecule. gRNAs that exist as a single RNA molecule may be referred to as single-guide RNAs (sgRNAs), though “gRNA” is used interchangeably to refer to guide RNAs that exist as either single molecules or as a complex of two or more molecules. Typically, gRNAs that exist as single RNA species comprise two domains: (1) a domain that shares homology to a target nucleic acid (e.g., and directs binding of a Cas9 (or equivalent) complex to the target); and (2) a domain that binds a Cas9 protein. In some embodiments, domain (2) corresponds to a sequence known as a tracrRNA, and comprises a stem-loop structure. For example, in some embodiments, domain (2) is homologous to a tracrRNA as depicted in FIG. 1E of Jinek et al., Science 337:816-821(2012), the entire contents of which is incorporated herein by reference. Other examples of gRNAs (e.g., those including domain 2) can be found in U.S. Pat. No. 9,340,799, entitled “mRNA-Sensing Switchable gRNAs,” and International Patent Application No. PCT/US2014/054247, filed Sep. 6, 2013, published as WO 2015/035136 and entitled “Delivery System For Functional Nucleases,” the entire contents of each are herein incorporated by reference. In some embodiments, a gRNA comprises two or more of domains (1) and (2), and may be referred to as an “extended gRNA.” For example, an extended gRNA will, e.g., bind two or more Cas9 proteins and bind a target nucleic acid at two or more distinct regions, as described herein. The gRNA comprises a nucleotide sequence that complements a target site, which mediates binding of the nuclease/RNA complex to said target site, providing the sequence specificity of the nuclease:RNA complex. In some embodiments, the RNA-programmable nuclease is the (CRISPR-associated system) Cas9 endonuclease, for example Cas9 (Csn1) from Streptococcus pyogenes (see, e.g., “Complete genome sequence of an M1 strain of Streptococcus pyogenes.” Ferretti J. J. et al., Proc. Natl. Acad. Sci. U.S.A. 98:4658-4663(2001); “CRISPR RNA maturation by trans-encoded small RNA and host factor RNase III.” Deltcheva E. et al., Nature 471:602-607(2011); and “A programmable dual-RNA-guided DNA endonuclease in adaptive bacterial immunity.” Jinek M. et al., Science 337:816-821(2012), the entire contents of each of which are incorporated herein by reference.

The napDNAbp nucleases (e.g., Cas9) use RNA:DNA hybridization to target DNA cleavage sites, these proteins are able to be targeted, in principle, to any sequence specified by the guide RNA. Methods of using napDNAbp nucleases, such as Cas9, for site-specific cleavage (e.g., to modify a genome) are known in the art (see e.g., Cong, L. et al. Multiplex genome engineering using CRISPR/Cas systems. Science 339, 819-823 (2013); Mali, P. et al. RNA-guided human genome engineering via Cas9. Science 339, 823-826 (2013); Hwang, W. Y. et al. Efficient genome editing in zebrafish using a CRISPR-Cas system. Nature Biotechnology 31, 227-229 (2013); Jinek, M. et al. RNA-programmed genome editing in human cells. eLife 2, e00471 (2013); Dicarlo, J. E. et al., Genome engineering in Saccharomyces cerevisiae using CRISPR-Cas systems. Nucleic Acid Res. (2013); Jiang, W. et al. RNA-guided editing of bacterial genomes using CRISPR-Cas systems. Nature Biotechnology 31, 233-239 (2013); the entire contents of each of which are incorporated herein by reference).

### Nickase

The term “nickase” refers to a napDNAbp having only a single nuclease activity that cuts only one strand of a target DNA, rather than both strands. Thus, a nickase type napDNAbp does not leave a double-strand break.

### Nuclear Localization Signal

A nuclear localization signal or sequence (NLS) is an amino acid sequence that tags, designates, or otherwise marks a protein for import into the cell nucleus by nuclear transport. Typically, this signal consists of one or more short sequences of positively charged lysines or arginines exposed on the protein surface. Different nuclear localized proteins may share the same NLS. An NLS has the opposite function of a nuclear export signal (NES), which targets proteins out of the nucleus. Thus, a single nuclear localization signal can direct the entity with which it is associated to the nucleus of a cell. Such sequences may be of any size and composition, for example more than 25, 25, 15, 12, 10, 8, 7, 6, 5, or 4 amino acids, but will preferably comprise at least a four to eight amino acid sequence known to function as a nuclear localization signal (NLS).

### Nucleic Acid Molecule

The term “nucleic acid molecule” as used herein, refers to RNA as well as single and/or double-stranded DNA. Nucleic acid molecules may be naturally occurring, for example, in the context of a genome, a transcript, an mRNA, tRNA, rRNA, siRNA, snRNA, a plasmid, cosmid, chromosome, chromatid, or other naturally occurring nucleic acid molecule. On the other hand, a nucleic acid molecule may be a non-naturally occurring molecule, e.g. a recombinant DNA or RNA, an artificial chromosome, an engineered genome, or fragment thereof, or a synthetic DNA, RNA, DNA/RNA hybrid, or including non-naturally occurring nucleotides or nucleosides. Furthermore, the terms “nucleic acid,” “DNA,” “RNA,” and/or similar terms include nucleic acid analogs, e.g. analogs having other than a phosphodiester backbone. Nucleic acids may be purified from natural sources, produced using recombinant expression systems and optionally purified, chemically synthesized, etc. Where appropriate, e.g. in the case of chemically synthesized molecules, nucleic acids may comprise nucleoside analogs such as analogs having chemically modified bases or sugars, and backbone modifications. A nucleic acid sequence is presented in the 5′ to 3′ direction unless otherwise indicated. In some embodiments, a nucleic acid is or comprises natural nucleosides (e.g. adenosine, thymidine, guanosine, cytidine, uridine, deoxyadenosine, deoxythymidine, deoxyguanosine, and deoxycytidine); nucleoside analogs (e.g. 2-aminoadenosine, 2-thiothymidine, inosine, pyrrolo-pyrimidine, 3-methyl adenosine, 5-methylcytidine, 2-aminoadenosine, C5-bromouridine, C5-fluorouridine, C5-iodouridine, C5-propynyl-uridine, C5-propynyl-cytidine, C5-methylcytidine, 2-aminoadenosine, 7-deazaadenosine, 7-deazaguanosine, inosinedenosine, 8-oxoguanosine, O(6)-methylguanine, and 2-thiocytidine); chemically modified bases; biologically modified bases (e.g. methylated bases); intercalated bases; modified sugars (e.g. 2′-fluororibose, ribose, 2′-deoxyribose, arabinose, and hexose); and/or modified phosphate groups (e.g. phosphorothioates and 5′-N-phosphoramidite linkages).

### PACE

The term “phage-assisted continuous evolution (PACE),” as used herein, refers to continuous evolution that employs phage as viral vectors. The general concept of PACE technology has been described, for example, in International PCT Application, PCT/US2009/056194, filed Sep. 8, 2009, published as WO 2010/028347 on Mar. 11, 2010; International PCT Application, PCT/US2011/066747, filed Dec. 22, 2011, published as WO 2012/088381 on Jun. 28, 2012; U.S. application, U.S. Pat. No. 9,023,594, issued May 5, 2015, International PCT Application, PCT/US2015/012022, filed Jan. 20, 2015, published as WO 2015/134121 on Sep. 11, 2015, and International PCT Application, PCT/US2016/027795, filed Apr. 15, 2016, published as WO 2016/168631 on Oct. 20, 2016, the entire contents of each of which are incorporated herein by reference.

### Promoter

The term “promoter” is art-recognized and refers to a nucleic acid molecule with a sequence recognized by the cellular transcription machinery and able to initiate transcription of a downstream gene. A promoter may be constitutively active, meaning that the promoter is always active in a given cellular context, or conditionally active, meaning that the promoter is only active in the presence of a specific condition. For example, a conditional promoter may only be active in the presence of a specific protein that connects a protein associated with a regulatory element in the promoter to the basic transcriptional machinery, or only in the absence of an inhibitory molecule. A subclass of conditionally active promoters is inducible promoters that require the presence of a small molecule “inducer” for activity. Examples of inducible promoters include, but are not limited to, arabinose-inducible promoters, Tet-on promoters, and tamoxifen-inducible promoters. A variety of constitutive, conditional, and inducible promoters are well known to the skilled artisan, and the skilled artisan will be able to ascertain a variety of such promoters useful in carrying out the instant invention, which is not limited in this respect. In various embodiments, the disclosure provides vectors with appropriate promoters for driving expression of the nucleic acid sequences encoding the fusion proteins (or one or more individual components thereof).

### Protein, Peptide, and Polypeptide

The terms “protein,” “peptide,” and “polypeptide” are used interchangeably herein, and refer to a polymer of amino acid residues linked together by peptide (amide) bonds. The terms refer to a protein, peptide, or polypeptide of any size, structure, or function. Typically, a protein, peptide, or polypeptide will be at least three amino acids long. A protein, peptide, or polypeptide may refer to an individual protein or a collection of proteins. One or more of the amino acids in a protein, peptide, or polypeptide may be modified, for example, by the addition of a chemical entity such as a carbohydrate group, a hydroxyl group, a phosphate group, a farnesyl group, an isofarnesyl group, a fatty acid group, a linker for conjugation, functionalization, or other modification, etc. A protein, peptide, or polypeptide may also be a single molecule or may be a multi-molecular complex. A protein, peptide, or polypeptide may be just a fragment of a naturally occurring protein or peptide. A protein, peptide, or polypeptide may be naturally occurring, recombinant, or synthetic, or any combination thereof. The term “fusion protein” as used herein refers to a hybrid polypeptide which comprises protein domains from at least two different proteins. One protein may be located at the amino-terminal (N-terminal) portion of the fusion protein or at the carboxy-terminal (C-terminal) protein thus forming an “amino-terminal fusion protein” or a “carboxy-terminal fusion protein,” respectively. A protein may comprise different domains, for example, a nucleic acid binding domain (e.g., the gRNA binding domain of Cas9 that directs the binding of the protein to a target site) and a nucleic acid cleavage domain or a catalytic domain of a recombinase. In some embodiments, a protein comprises a proteinaceous part, e.g., an amino acid sequence constituting a nucleic acid binding domain, and an organic compound, e.g., a compound that can act as a nucleic acid cleavage agent. In some embodiments, a protein is in a complex with, or is in association with, a nucleic acid, e.g., RNA. Any of the proteins provided herein may be produced by any method known in the art. For example, the proteins provided herein may be produced via recombinant protein expression and purification, which is especially suited for fusion proteins comprising a peptide linker. Methods for recombinant protein expression and purification are well known, and include those described by Green and Sambrook, Molecular Cloning: A Laboratory Manual (4th ed., Cold Spring Harbor Laboratory Press, Cold Spring Harbor, N.Y. (2012)), the entire contents of which are incorporated herein by reference. It should be appreciated that the disclosure provides any of the polypeptide sequences provided herein without an N-terminal methionine (M) residue.

### RNA-Protein Recruitment System

In various embodiments, two separate protein domains (e.g., a Cas9 domain and a cytidine deaminase domain) may be colocalized to one another to form a functional complex (akin to the function of a fusion protein comprising the two separate protein domains) by using an “RNA-protein recruitment system,” such as the “MS2 tagging technique.” Such systems generally tag one protein domain with an “RNA-protein interaction domain” (aka “RNA-protein recruitment domain”) and the other with an “RNA-binding protein” that specifically recognizes and binds to the RNA-protein interaction domain, e.g., a specific hairpin structure. These types of systems can be leveraged to colocalize the domains of a base editor, as well as to recruitment additional functionalities to a base editor, such as a UGI domain. In one example, the MS2 tagging technique is based on the natural interaction of the MS2 bacteriophage coat protein (“MCP” or “MS2cp”) with a stem-loop or hairpin structure present in the genome of the phage, i.e., the “MS2 hairpin.” In the case of the MS2 hairpin, it is recognized and bound by the MS2 bacteriophage coat protein (MCP). Thus, in one exemplary scenario a deaminase-MS2 fusion can recruit a Cas9-MCP fusion.

A review of other modular RNA-protein interaction domains are described in the art, for example, in Johansson et al., “RNA recognition by the MS2 phage coat protein,” Sem Virol., 1997, Vol. 8(3): 176-185; Delebecque et al., “Organization of intracellular reactions with rationally designed RNA assemblies,” Science, 2011, Vol. 333: 470-474; Mali et al., “Cas9 transcriptional activators for target specificity screening and paired nickases for cooperative genome engineering,” Nat. Biotechnol., 2013, Vol. 31: 833-838; and Zalatan et al., “Engineering complex synthetic transcriptional programs with CRISPR RNA scaffolds,” Cell, 2015, Vol. 160: 339-350, each of which are incorporated herein by reference in their entireties. Other systems include the PP7 hairpin, which specifically recruits the PCP protein, and the “com” hairpin, which specifically recruits the Com protein. See Zalatan et al.

The nucleotide sequence of the MS2 hairpin (or equivalently referred to as the “MS2 aptamer”) is: GCCAACATGAGGATCACCCATGTCTGCAGGGCC (SEQ ID NO: 172).

The amino acid sequence of the MCP or MS2cp is:

(SEQ ID NO: 173)
GSASNFTQFVLVDNGGTGDVTVAPSNFANGVAEWISSNSRSQAYKVTCSV
RQSSAQNRKYTIKVEVPKVATQTVGGEELPVAGWRSYLNMELTIPIFATN
SDCELIVKAMQGLLKDGNPIPSAIAANSGIY.

### Sense Strand

In genetics, a “sense” strand is the segment within double-stranded DNA that runs from 5′ to 3′, and which is complementary to the antisense strand of DNA, or template strand, which runs from 3′ to 5′. In the case of a DNA segment that encodes a protein, the sense strand is the strand of DNA that has the same sequence as the mRNA, which takes the antisense strand as its template during transcription, and eventually undergoes (typically, not always) translation into a protein. The antisense strand is thus responsible for the RNA that is later translated to protein, while the sense strand possesses a nearly identical makeup to that of the mRNA. Note that for each segment of dsDNA, there will possibly be two sets of sense and antisense, depending on which direction one reads (since sense and antisense is relative to perspective). It is ultimately the gene product, or mRNA, that dictates which strand of one segment of dsDNA is referred to as sense or antisense.

In the context of a PEgRNA, the first step is the synthesis of a single-strand complementary DNA (i.e., the 3′ ssDNA flap, which becomes incorporated) oriented in the 5′ to 3′ direction which is templated off of the PEgRNA extension arm. Whether the 3′ ssDNA flap should be regarded as a sense or antisense strand depends on the direction of transcription since it well accepted that both strands of DNA may serve as a template for transcription (but not at the same time). Thus, in some embodiments, the 3′ ssDNA flap (which overall runs in the 5′ to 3′ direction) will serve as the sense strand because it is the coding strand. In other embodiments, the 3′ ssDNA flap (which overall runs in the 5′ to 3′ direction) will serve as the antisense strand and thus, the template for transcription.

### Subject

The term “subject,” as used herein, refers to an individual organism, for example, an individual mammal. In some embodiments, the subject is a human. In some embodiments, the subject is a non-human mammal. In some embodiments, the subject is a non-human primate. In some embodiments, the subject is a rodent. In some embodiments, the subject is a sheep, a goat, a cattle, a cat, or a dog. In some embodiments, the subject is a vertebrate, an amphibian, a reptile, a fish, an insect, a fly, or a nematode. In some embodiments, the subject is a research animal. In some embodiments, the subject is genetically engineered, e.g., a genetically engineered non-human subject. The subject may be of either sex and at any stage of development.

### Target Site

The term “target site” refers to a sequence within a nucleic acid molecule that is edited by a fusion protein (e.g. a dCas9-deaminase fusion protein provided herein). The target site further refers to the sequence within a nucleic acid molecule to which a complex of the fusion protein and gRNA binds.

### Transcription Terminator

A “transcriptional terminator” is a nucleic acid sequence that causes transcription to stop. A transcriptional terminator may be unidirectional or bidirectional. It is comprised of a DNA sequence involved in specific termination of an RNA transcript by an RNA polymerase. A transcriptional terminator sequence prevents transcriptional activation of downstream nucleic acid sequences by upstream promoters. A transcriptional terminator may be necessary in vivo to achieve desirable expression levels or to avoid transcription of certain sequences. A transcriptional terminator is considered to be “operably linked to” a nucleotide sequence when it is able to terminate the transcription of the sequence it is linked to.

The most commonly used type of terminator is a forward terminator. When placed downstream of a nucleic acid sequence that is usually transcribed, a forward transcriptional terminator will cause transcription to abort. In some embodiments, bidirectional transcriptional terminators are provided, which usually cause transcription to terminate on both the forward and reverse strand. In some embodiments, reverse transcriptional terminators are provided, which usually terminate transcription on the reverse strand only.

In prokaryotic systems, terminators usually fall into two categories (1) rho-independent terminators and (2) rho-dependent terminators. Rho-independent terminators are generally composed of palindromic sequence that forms a stem loop rich in G-C base pairs followed by several T bases. Without wishing to be bound by theory, the conventional model of transcriptional termination is that the stem loop causes RNA polymerase to pause, and transcription of the poly-A tail causes the RNA:DNA duplex to unwind and dissociate from RNA polymerase.

In eukaryotic systems, the terminator region may comprise specific DNA sequences that permit site-specific cleavage of the new transcript so as to expose a polyadenylation site. This signals a specialized endogenous polymerase to add a stretch of about 200 A residues (polyA) to the 3′ end of the transcript. RNA molecules modified with this polyA tail appear to more stable and are translated more efficiently. Thus, in some embodiments involving eukaryotes, a terminator may comprise a signal for the cleavage of the RNA. In some embodiments, the terminator signal promotes polyadenylation of the message. The terminator and/or polyadenylation site elements may serve to enhance output nucleic acid levels and/or to minimize read through between nucleic acids.

Terminators for use in accordance with the present disclosure include any terminator of transcription described herein or known to one of ordinary skill in the art. Examples of terminators include, without limitation, the termination sequences of genes such as, for example, the bovine growth hormone terminator, and viral termination sequences such as, for example, the SV40 terminator, spy, yejM, secG-leuU, thrLABC, rrnB T1, hisLGDCBHAFI, metZWV, rrnC, xapR, aspA and arcA terminator. In some embodiments, the termination signal may be a sequence that cannot be transcribed or translated, such as those resulting from a sequence truncation.

### Transition

As used herein, “transitions” refer to the interchange of purine nucleobases (A↔G) or the interchange of pyrimidine nucleobases (C↔T). This class of interchanges involves nucleobases of similar shape. The compositions and methods disclosed herein are capable of inducing one or more transitions in a target DNA molecule. The compositions and methods disclosed herein are also capable of inducing both transitions and transversion in the same target DNA molecule. These changes involve A↔G, G↔A, C↔T, or T↔C. In the context of a double-strand DNA with Watson-Crick paired nucleobases, transversions refer to the following base pair exchanges: A:T↔G:C, G:G↔A:T, C:G↔T:A, or T:A↔C:G. The compositions and methods disclosed herein are capable of inducing one or more transitions in a target DNA molecule. The compositions and methods disclosed herein are also capable of inducing both transitions and transversion in the same target DNA molecule, as well as other nucleotide changes, including deletions and insertions.

### Transversion

As used herein, “transversions” refer to the interchange of purine nucleobases for pyrimidine nucleobases, or in the reverse and thus, involve the interchange of nucleobases with dissimilar shape. These changes involve T↔A, T↔G, C↔G, C↔A, A↔T, A↔C, G↔C, and G↔T. In the context of a double-strand DNA with Watson-Crick paired nucleobases, transversions refer to the following base pair exchanges: T:A↔A:T, T:A↔G:C, C:G↔G:C, C:G↔A:T, A:T↔T:A, A:T↔C:G, G:C↔C:G, and G:C↔T:A. The compositions and methods disclosed herein are capable of inducing one or more transversions in a target DNA molecule. The compositions and methods disclosed herein are also capable of inducing both transitions and transversion in the same target DNA molecule, as well as other nucleotide changes, including deletions and insertions.

### Treatment

The terms “treatment,” “treat,” and “treating,” refer to a clinical intervention aimed to reverse, alleviate, delay the onset of, or inhibit the progress of a disease or disorder, or one or more symptoms thereof, as described herein. As used herein, the terms “treatment,” “treat,” and “treating” refer to a clinical intervention aimed to reverse, alleviate, delay the onset of, or inhibit the progress of a disease or disorder, or one or more symptoms thereof, as described herein. In some embodiments, treatment may be administered after one or more symptoms have developed and/or after a disease has been diagnosed. In other embodiments, treatment may be administered in the absence of symptoms, e.g., to prevent or delay onset of a symptom or inhibit onset or progression of a disease. For example, treatment may be administered to a susceptible individual prior to the onset of symptoms (e.g., in light of a history of symptoms and/or in light of genetic or other susceptibility factors). Treatment may also be continued after symptoms have resolved, for example, to prevent or delay their recurrence.

### Upstream

As used herein, the terms “upstream” and “downstream” are terms of relativety that define the linear position of at least two elements located in a nucleic acid molecule (whether single or double-stranded) that is orientated in a 5′-to-3′ direction. In particular, a first element is upstream of a second element in a nucleic acid molecule where the first element is positioned somewhere that is 5′ to the second element. For example, a SNP is upstream of a Cas9-induced nick site if the SNP is on the 5′ side of the nick site. Conversely, a first element is downstream of a second element in a nucleic acid molecule where the first element is positioned somewhere that is 3′ to the second element. For example, a SNP is downstream of a Cas9-induced nick site if the SNP is on the 3′ side of the nick site. The nucleic acid molecule can be a DNA (double or single stranded). RNA (double or single stranded), or a hybrid of DNA and RNA. The analysis is the same for single strand nucleic acid molecule and a double strand molecule since the terms upstream and downstream are in reference to only a single strand of a nucleic acid molecule, except that one needs to select which strand of the double stranded molecule is being considered. Often, the strand of a double stranded DNA which can be used to determine the positional relativity of at least two elements is the “sense” or “coding” strand. In genetics, a “sense” strand is the segment within double-stranded DNA that runs from 5′ to 3′, and which is complementary to the antisense strand of DNA, or template strand, which runs from 3′ to 5′. Thus, as an example, a SNP nucleobase is “downstream” of a promoter sequence in a genomic DNA (which is double-stranded) if the SNP nucleobase is on the 3′ side of the promoter on the sense or coding strand.

### Uracil Glycosylase Inhibitor

The term “uracil glycosylase inhibitor” or “UGI,” as used herein, refers to a protein that is capable of inhibiting a uracil-DNA glycosylase base-excision repair enzyme. In some embodiments, a UGI domain comprises a wild-type UGI or a UGI as set forth in SEQ ID NO: 163. In some embodiments, the UGI proteins provided herein include fragments of UGI and proteins homologous to a UGI or a UGI fragment. For example, in some embodiments, a UGI domain comprises a fragment of the amino acid sequence set forth in SEQ ID NO: 163. In some embodiments, a UGI fragment comprises an amino acid sequence that comprises at least 60%, at least 65%, at least 70%, at least 75%, at least 80%, at least 85%, at least 90%, at least 95%, at least 96%, at least 97%, at least 98%, at least 99%, or at least 99.5% of the amino acid sequence as set forth in SEQ ID NO: 163. In some embodiments, a UGI comprises an amino acid sequence homologous to the amino acid sequence set forth in SEQ ID NO: 163, or an amino acid sequence homologous to a fragment of the amino acid sequence set forth in SEQ ID NO: 163. In some embodiments, proteins comprising UGI or fragments of UGI or homologs of UGI or UGI fragments are referred to as “UGI variants.” A UGI variant shares homology to UGI, or a fragment thereof. For example a UGI variant is at least 70% identical, at least 75% identical, at least 80% identical, at least 85% identical, at least 90% identical, at least 95% identical, at least 96% identical, at least 97% identical, at least 98% identical, at least 99% identical, at least 99.5% identical, or at least 99.9% identical to a wild type UGI or a UGI as set forth in SEQ ID NO: 163. In some embodiments, the UGI variant comprises a fragment of UGI, such that the fragment is at least 70% identical, at least 80% identical, at least 90% identical, at least 95% identical, at least 96% identical, at least 97% identical, at least 98% identical, at least 99% identical, at least 99.5% identical, or at least 99.9% to the corresponding fragment of wild-type UGI or a UGI as set forth in SEQ ID NO: 163. In some embodiments, the UGI comprises the following amino acid sequence:

(SEQ ID NO: 163)
MTNLSDIIEKETGKQLVIQESILMLPEEVEEVIGNKPESDILVHTAYDES
TDENVMLLTSDAPEYKPWALVIQDSNGENKIKML
(P14739|UNGI_BPPB2 Uracil-DNA glycosylase
inhibitor).

### Variant

As used herein, the term “variant” refers to a protein having characteristics that deviate from what occurs in nature that retains at least one functional i.e. binding, interaction, or enzymatic ability and/or therapeutic property thereof. A “variant” is at least about 70% identical, at least about 80% identical, at least about 90% identical, at least about 95% identical, at least about 96% identical, at least about 97% identical, at least about 98% identical, at least about 99% identical, at least about 99.5% identical, or at least about 99.9% identical to the wild type protein. For instance, a variant of Cas9 may comprise a Cas9 that has one or more changes in amino acid residues as compared to a wild type Cas9 amino acid sequence. As another example, a variant of a deaminase may comprise a deaminase that has one or more changes in amino acid residues as compared to a wild type deaminase amino acid sequence, e.g. following ancestral sequence reconstruction of the deaminase. These changes include chemical modifications, including substitutions of different amino acid residues truncations, covalent additions (e.g. of a tag), and any other mutations. The term also encompasses circular permutants, mutants, truncations, or domains of a reference sequence, and which display the same or substantially the same functional activity or activities as the reference sequence. This term also embraces fragments of a wild type protein.

The level or degree of which the property is retained may be reduced relative to the wild type protein but is typically the same or similar in kind. Generally, variants are overall very similar, and in many regions, identical to the amino acid sequence of the protein described herein. A skilled artisan will appreciate how to make and use variants that maintain all, or at least some, of a functional ability or property.

The variant proteins may comprise, or alternatively consist of, an amino acid sequence which is at least 80%, 85%, 90%, 95%, 96%, 97%, 98%, 99%, or 100%, identical to, for example, the amino acid sequence of a wild-type protein, or any protein provided herein (e.g. SMN protein).

By a polypeptide having an amino acid sequence at least, for example, 95% “identical” to a query amino acid sequence, it is intended that the amino acid sequence of the subject polypeptide is identical to the query sequence except that the subject polypeptide sequence may include up to five amino acid alterations per each 100 amino acids of the query amino acid sequence. In other words, to obtain a polypeptide having an amino acid sequence at least 95% identical to a query amino acid sequence, up to 5% of the amino acid residues in the subject sequence may be inserted, deleted, or substituted with another amino acid. These alterations of the reference sequence may occur at the amino- or carboxy-terminal positions of the reference amino acid sequence or anywhere between those terminal positions, interspersed either individually among residues in the reference sequence or in one or more contiguous groups within the reference sequence.

As a practical matter, whether any particular polypeptide is at least 80%, 85%, 90%, 95%, 96%, 97%, 98%, or 99% identical to, for instance, the amino acid sequence of a protein such as a SMN protein, can be determined conventionally using known computer programs. A preferred method for determining the best overall match between a query sequence (a sequence of the present invention) and a subject sequence, also referred to as a global sequence alignment, can be determined using the FASTDB computer program based on the algorithm of Brutlag et al. (Comp. App. Biosci. 6:237-245 (1990)). In a sequence alignment the query and subject sequences are either both nucleotide sequences or both amino acid sequences. The result of said global sequence alignment is expressed as percent identity. Preferred parameters used in a FASTDB amino acid alignment are: Matrix=PAM 0, k-tuple=2, Mismatch Penalty=1, Joining Penalty=20, Randomization Group Length=0, Cutoff Score=1, Window Size=sequence length, Gap Penalty=5, Gap Size Penalty=0.05, Window Size=500 or the length of the subject amino acid sequence, whichever is shorter.

If the subject sequence is shorter than the query sequence due to N- or C-terminal deletions, not because of internal deletions, a manual correction must be made to the results. This is because the FASTDB program does not account for N- and C-terminal truncations of the subject sequence when calculating global percent identity. For subject sequences truncated at the N- and C-termini, relative to the query sequence, the percent identity is corrected by calculating the number of residues of the query sequence that are N- and C-terminal of the subject sequence, which are not matched/aligned with a corresponding subject residue, as a percent of the total bases of the query sequence. Whether a residue is matched/aligned is determined by results of the FASTDB sequence alignment. This percentage is then subtracted from the percent identity, calculated by the above FASTDB program using the specified parameters, to arrive at a final percent identity score. This final percent identity score is what is used for the purposes of the present invention. Only residues to the N- and C-termini of the subject sequence, which are not matched/aligned with the query sequence, are considered for the purposes of manually adjusting the percent identity score. That is, only query residue positions outside the farthest N- and C-terminal residues of the subject sequence.

### Vector

The term “vector,” as used herein, refers to a nucleic acid that can be modified to encode a gene of interest and that is able to enter into a host cell, mutate and replicate within the host cell, and then transfer a replicated form of the vector into another host cell. Exemplary suitable vectors include viral vectors, such as retroviral vectors or bacteriophages and filamentous phage, and conjugative plasmids. Additional suitable vectors will be apparent to those of skill in the art based on the instant disclosure.

### Wild Type

As used herein the term “wild type” is a term of the art understood by skilled persons and means the typical form of an organism, strain, gene or characteristic as it occurs in nature as distinguished from mutant or variant forms.

These and other exemplary substituents are described in more detail in the Detailed Description, Examples, and claims.

### DETAILED DESCRIPTION OF CERTAIN EMBODIMENTS

The present disclosure provides a novel machine learning algorithm capable of assisting those of ordinary skill in the art to conduct base editing by, inter alia, facilitating the selection of an appropriate guide RNA and base editor combination which are capable of conducting base editing at a certain level of efficiency and specificity on a given input target DNA sequence desired to be edited to produce an outcome genotype of interest. The novel machine learning algorithm described and claimed herein can be referred to as “BE-Hive.” The disclosure further provides a graphical user interface that implements BE-Hive, allowing a user to input various features, including a desired target DNA sequence, an appropriate guide RNA (or associated CRISPR protospacer), a base editor, and a cell in which base editing is to take place, and to predict base editing efficiencies and bystander editing patterns for the selected features.

The utility of base editing has inspired the development of many cytosine and adenine base editor variants with distinct editing properties (Adli, 2018; Molla and Yang, 2019; Rees and Liu, 2018). To date, these properties have been gleaned by analyzing base editing outcomes at a modest number of genomic sites, often chosen to align with previous genome editing studies (Gaudelli et al., 2017; Gehrke et al., 2018; Huang et al., 2019; Komor et al., 2016; Thuronyi et al., 2019). The interplay between base editor and target sequence, however, influences base editing outcomes in complex and occasionally unintuitive ways (Gehrke et al., 2018; Huang et al., 2019; Tan et al., 2019; Thuronyi et al., 2019; Villiger et al., 2018). As a result, obtaining a desired genotype with useful efficiencies often requires empirical optimization of base editor and single guide RNA (sgRNA) choice for each target. Likewise, some viable targets that do not fit canonical guidelines for base editing use may be overlooked since simple guidelines for target selection likely do not fully capture the scope of base editing. A systematic and comprehensive analysis of sequence and deaminase determinants of base editing thus would enhance the understanding of base editors, facilitate their use in precision editing applications, and guide development of new base editors with enhanced abilities to induce or prevent rare base editing outcomes.

As described herein in certain embodiments, libraries of 38,538 total pairs of sgRNAs and target sequences were developed and integrated into three mammalian cell types to comprehensively characterize base editing outcomes and sequence-activity relationships for eight popular cytosine and adenine base editors in living cells. The roles of deaminases, sequence context, and cell type in determining genotypes that result from base editing were analyzed, and a machine learning algorithm was developed that accurately predicts base editing outcomes, including many previously unpredictable features, at any target site of interest. Using the resulting information, a variety of base editors were applied, including newly engineered variants, to precisely correct 3,388 genotypes and 2,399 coding sequences of disease-associated SNVs to wild-type with ≥90% precision among edited products, including by previously poorly understood non-canonical base editing outcomes. The herein disclosed and claimed machine learning algorithm facilitates the selection of an appropriate guide RNA and base editor combination which are capable of conducting base editing at a certain level of efficiency and specificity on a given input target DNA sequence desired to be edited to produce an outcome genotype of interest.

In various aspects, the instant specification describes machine learning algorithms for selecting guide RNAs for base editing based on a particular base editor and other determinants of base editing, which include, but are not limited to the choice of the napDNAbp of the base editing system; the choice of the deaminase of the base editing system; the nucleotide sequence; the target genomic location; the transcriptional state of the target genomic location; locus-dependent activity of the choice napDNAbp; cell-type; transcriptional state of DNA repair proteins; and base editor modifications. The disclosure also provides machine learning algorithms for predicting genotype outcomes based on a particular base editor and other determinants of base editing, which include, but are not limited to the choice of the napDNAbp of the base editing system; the choice of the deaminase of the base editing system; the nucleotide sequence; the target genomic location; the transcriptional state of the target genomic location; locus-dependent activity of the choice napDNAbp; cell-type; transcriptional state of DNA repair proteins; and base editor modifications. The disclosure further provides base editors (e.g., ABEs and CBEs), napDNAbps, cytidine deaminases, adenosine deaminases, nucleic acid sequences encoding base editors and components thereof, vectors, and cells. In addition, the disclosure provides methods of making biological or experimental training and/or validation data for training and/or validating the machine learning computational models, as well as, vectors, libraries, and nucleic acid sequences for use in obtaining said experimental training and/or validation data, as well as the experimental training data and/or validation data itself.

The machine learning algorithm considers various inputs, including the sequence of the target DNA sequence to be edited, the napDNAbp options, the deaminase options, the guide RNA options, the spacer and/or protospacer sequence associated with the RNA options, dinucleotide composition at neighboring positions in the protospacers, guide RNA melting temperatures, and the total number of G, C, A, and/or T nucleotides in the protospacer sequence, among other features. In addition, other features that may be considered as input to the machine learning algorithm. Such features may include, but are not limited to, the transcriptional state of the target genomic location, cell-type in which the base editing is taking place, transcriptional state of the target DNA being edited, and any epigenetic modifications of the target DNA being edited.

The disclosure further provides base editors (e.g., ABEs and CBEs), napDNAbps, cytidine deaminases, adenosine deaminases, nucleic acid sequences encoding base editors and/or guide RNAs, vectors, and cells. In other aspects, the disclosure provides guide RNA sequences (and/or spacer sequences or protospacer sequences associated therewith) that can be selected and/or identified by the machine learning algorithm described herein, as well as compositions comprising said guide RNA sequences and a base editor for editing a target DNA sequence (e.g., correcting a point mutation). In addition, the disclosure provides methods of making biological or experimental training and/or validation data for training and/or validating the machine learning algorithms described herein, as well as, vectors, libraries, and nucleic acid sequences for use in obtaining said experimental training and/or validation data, as well as the experimental training data and/or validation data itself.

In one aspect, the disclosure provides a method of using at least one machine learning model to identify a guide RNA for use in a base editing system for introducing a target change into a nucleotide sequence, the base editing system comprising a napDNAbp and a deaminase, the method comprising using software executing on at least one computer hardware processor to perform: obtaining input data indicative of the nucleotide sequence and a set of one or more guide RNAs; generating first input features from the input data; applying a first machine learning model to the first input features to obtain first output data indicative, for each one guide RNA in the set of guide RNAs, of a base editing efficiency, at one or multiple locations in the nucleotide sequence, of the base editing system when using the each one guide RNA; generating second input features from the input data; applying a second machine learning model to the second input features to obtain second output data indicative, for each one guide RNA in the set of guide RNAs, of bystander editing activity, at one or multiple locations in the nucleotide sequence, by the base editing system when using the each one guide RNA; and identifying, using the first output data and the second output data, the guide RNA for use in the base editing system for introducing the target change into the nucleotide sequence.

In certain embodiments, the set of guide RNAs includes a first guide RNA, and wherein, the input data includes first data indicative of at least a part of a nucleotide sequence associated with the first guide RNA.

The first data can specify a spacer or a protospacer sequence associated with the first guide RNA.

The step of obtaining the data indicative of the nucleotide sequence and the set of guide RNAs, can comprise: obtaining, by the software and from at least one source external to the software, the data indicative of the nucleotide sequence and the set of guide RNAs.

The step of obtaining the data indicative of the nucleotide sequence and the set of guide RNAs, comprises: obtaining, by the software and from at least one source external to the software, first data indicative of the nucleotide sequence; and generating, from the first data indicative of the nucleotide sequence, data indicative of the set of guide RNAs.

In certain embodiments, the first machine learning model comprises a non-linear machine learning model selected from the group consisting of a random forest model, a logistic regression model, a support vector machine model, a generalized linear model, a hierarchical Bayesian model, and neural network model.

In other embodiments, the first machine learning model can comprise a random forest model.

The set of guide RNAs can include a first guide RNA, and wherein generating the first input features comprises generating multiple features to include in the first input features, the multiple features including: features encoding at least some nucleotides in a protospacer sequence or spacer sequence associated with the first guide RNA; and features encoding at least some nucleotides, in the nucleotide sequence, located within a threshold number of nucleotides of the protospacer sequence associated with the first guide RNA.

The step of generating the features encoding the at least some nucleotides in the protospacer sequence comprises generating a one-hot encoding of the at least some nucleotides in the protospacer sequence.

In various embodiments, the multiple features further include one or more of the following features: features encoding at least some dinucleotides at neighboring positions in the protospacer sequence; features representing melting temperature of the first guide RNA; one or more features representing a total number of G, C, A, and/or T nucleotides in the protospacer sequence; and a feature representing an average base editing efficiency of the base editing system.

In certain embodiments, the set of guide RNAs includes a first guide RNA, wherein the first output data is indicative of a fraction of sequence reads containing at least one base edit at any nucleotide in a target window about a protospacer sequence associated with the first guide RNA, among all sequence reads.

In other embodiments, the second first machine learning model comprises a non-linear machine learning model selected from the group consisting of a random forest model, a logistic regression model, a support vector machine model, a generalized linear model, a hierarchical Bayesian model, and neural network model.

In yet other embodiments, the second machine learning model comprises a deep neural network model.

The neural network model can comprise a conditional autoregressive neural network model.

The conditional autoregressive neural network model can include: an encoder neural network mapping input data to a latent representation; and a decoder neural network mapping the latent representation to output data, wherein the decoder neural network has an autoregressive structure.

The encoder neural network can comprise a multi-layer fully connected network with residual connections.

The decoder neural network can generate a distribution over base editing outcomes at each nucleotide while conditioning on previously-generated outcomes.

The neural network model can include parameters representing a position-wise bias toward producing an unedited outcome.

The set of guide RNAs can include a first guide RNA, and wherein generating the second input features can comprise generating multiple features to include in the second input features, the multiple features including: features encoding at least some nucleotides in a protospacer sequence or spacer sequence associated with the first guide RNA; and features encoding at least some nucleotides, in the nucleotide sequence, located within a threshold number of nucleotides of the protospacer sequence associated with the first guide RNA.

In other embodiments, the second output data can be indicative of frequencies of occurrence of base editing outcomes each of which includes edits to nucleotides at multiple positions.

The second output data can be indicative of a frequency distribution on combinations of base editing outcomes.

In various embodiments, the set of guide RNAs can include a first guide RNA, wherein, for a specific combination of base edits, the second output data is indicative of a frequency of occurrence of the specific combination of base edits among all sequenced reads containing at least one base edit at any nucleotide in a target window about a protospacer sequence associated with the first guide RNA.

In other embodiments, the set of guide RNAs can include a first guide RNA, wherein the first output data includes a first base editing efficiency value for the first guide RNA, wherein the second output data includes a first bystander editing value for the first guide RNA, and wherein identifying the guide RNA using the first output data and the second output data, comprises multiplying the first base editing efficiency value by the first bystander editing value.

In certain embodiments, the first machine learning model comprises a first plurality of values for a respective first plurality of parameters, the first plurality of values used by the at least one computer hardware processor to obtain the first output data from the first input features.

The first plurality of parameters can comprise at least one thousand parameters.

The first plurality of parameters can comprise between one thousand and ten thousand parameters.

In various embodiments, the first machine learning model can comprise a random forest model comprising at least 100 decision trees, each of the at least 100 decision trees having at least a depth of D, and wherein processing the input data using the random forest model comprises performing 100*D comparisons.

The random forest model can comprise at least 500 decision trees.

In certain embodiments, depth of D can be greater than or equal to five, wherein processing the input data using the random forest model comprises performing at least 2500 comparisons.

In other embodiments, the second machine learning model can comprise a second plurality of values for a respective second plurality of parameters, the second plurality of values used by the at least one computer hardware processor to obtain the second output data from the second input features.

The second plurality of parameters can comprise at least ten thousand parameters, or between 25,000 and 100,000 parameters, or between 30,000 and 40,000 parameters.

In other embodiments, the disclosure provides a method of manufacturing the identified guide RNA for use in the base editing system for introducing the target change into the nucleotide sequence.

In other aspects, the disclosure provides a method for training the first machine learning model of any of the above aspects comprising: (i) preparing a library comprising a plurality of nucleic acid molecules each encoding a nucleotide target sequence and a cognate guide RNA; (ii) introducing the library into a plurality of host cells; (iii) contacting the library in the host cells with a Cas-based genome editing system to produce a plurality of genomic repair products; (iv) determining the sequences of the genomic repair products; and (v) training the first machine learning model with training data that comprises at least the sequences of the genomic repair products and the cognate guide RNA.

In still other embodiments, the disclosure provides a method for training the second machine learning model of any of the above aspects comprising: (i) preparing a library comprising a plurality of nucleic acid molecules each encoding a nucleotide target sequence and a cognate guide RNA; (ii) introducing the library into a plurality of host cells; (iii) contacting the library in the host cells with a Cas-based genome editing system to produce a plurality of genomic repair products; (iv) determining the sequences of the genomic repair products; and (v) training the second machine learning model with training data that comprises at least the sequences of the genomic repair products and the cognate guide RNA.

The disclosure also provides for a computer readable storage medium storing processor executable instructions that, when executed by at least one computer hardware processor, cause the at least one computer hardware processor to perform a method of using at least one machine learning model to identify a guide RNA for use in a base editing system for introducing a target change into a nucleotide sequence, the base editing system comprising a napDNAbp and a deaminase, the method comprising: obtaining input data indicative of the nucleotide sequence and a set of one or more guide RNAs; generating first input features from the input data; applying a first machine learning model to the first input features to obtain first output data indicative, for each one guide RNA in the set of guide RNAs, of a base editing efficiency, at one or multiple locations in the nucleotide sequence, of the base editing system when using the each one guide RNA; generating second input features from the input data; applying a second machine learning model to the second input features to obtain second output data indicative, for each one guide RNA in the set of guide RNAs, of bystander editing activity, at one or multiple locations in the nucleotide sequence, by the base editing system when using the each one guide RNA; and identifying, using the first output data and the second output data, the guide RNA for use in the base editing system for introducing the target change into the nucleotide sequence.

In another aspect, the disclosure provides a system comprising: at least one computer hardware processor; and at least one computer readable storage medium storing processor executable instructions that, when executed by the at least one computer hardware processor, cause the at least one computer hardware processor to perform a method of using at least one machine learning model to identify a guide RNA for use in a base editing system for introducing a target change into a nucleotide sequence, the base editing system comprising a napDNAbp and a deaminase, the method comprising: obtaining input data indicative of the nucleotide sequence and a set of one or more guide RNAs; generating first input features from the input data; applying a first machine learning model to the first input features to obtain first output data indicative, for each one guide RNA in the set of guide RNAs, of a base editing efficiency, at one or multiple locations in the nucleotide sequence, of the base editing system when using the each one guide RNA; generating second input features from the input data; applying a second machine learning model to the second input features to obtain second output data indicative, for each one guide RNA in the set of guide RNAs, of bystander editing activity, at one or multiple locations in the nucleotide sequence, by the base editing system when using the each one guide RNA; and identifying, using the first output data and the second output data, the guide RNA for use in the base editing system for introducing the target change into the nucleotide sequence.

In other aspects, the machine learning model can be based solely on the base editing efficiency machine learning model, for example, a method identifying a guide RNA for use in a base editing system for introducing a target change into a nucleotide sequence, the base editing system comprising a napDNAbp and a deaminase, the method comprising: using software executing on at least one computer hardware processor to perform: obtaining input data indicative of the nucleotide sequence and a set of one or more guide RNAs; generating first input features from the input data; applying a first machine learning model to the first input features to obtain first output data indicative, for each one guide RNA in the set of guide RNAs, of a base editing efficiency, at one or multiple locations in the nucleotide sequence, of the base editing system when using the each one guide RNA; and identifying, using the first output data, the guide RNA for use in the base editing system for introducing the target change into the nucleotide sequence.

Nevertheless, in such aspects, the machine learning model can further comprise a bystander model, comprising generating second input features from the input data; applying a second machine learning model to the second input features to obtain second output data indicative, for each one guide RNA in the set of guide RNAs, of bystander editing activity, at one or multiple locations in the nucleotide sequence, by the base editing system when using the each one guide RNA, wherein identifying the guide RNA is performed using the first output data and the second output data.

The disclosure also provides at least one computer readable storage medium storing processor executable instructions that, when executed by at least one computer hardware processor, cause the at least one computer hardware processor to perform a method of identifying a guide RNA for use in a base editing system for introducing a target change into a nucleotide sequence, the base editing system comprising a napDNAbp and a deaminase, the method comprising: obtaining input data indicative of the nucleotide sequence and a set of one or more guide RNAs; generating first input features from the input data; applying a first machine learning model to the first input features to obtain first output data indicative, for each one guide RNA in the set of guide RNAs, of a base editing efficiency, at one or multiple locations in the nucleotide sequence, of the base editing system when using the each one guide RNA; and identifying, using the first output data, the guide RNA for use in the base editing system for introducing the target change into the nucleotide sequence.

In other aspects, the disclosure provides a system, comprising: at least one computer hardware processor; and at least one computer readable storage medium storing processor executable instructions that, when executed by the at least one computer hardware processor, cause the at least one computer hardware processor to perform a method of identifying a guide RNA for use in a base editing system for introducing a target change into a nucleotide sequence, the base editing system comprising a napDNAbp and a deaminase, the method comprising: obtaining input data indicative of the nucleotide sequence and a set of one or more guide RNAs; generating first input features from the input data; applying a first machine learning model to the first input features to obtain first output data indicative, for each one guide RNA in the set of guide RNAs, of a base editing efficiency, at one or multiple locations in the nucleotide sequence, of the base editing system when using the each one guide RNA; and identifying, using the first output data, the guide RNA for use in the base editing system for introducing the target change into the nucleotide sequence.

In other aspects, the machine learning model can be based solely on the bystander machine learning model, comprising a method of identifying a guide RNA for use in a base editing system for introducing a target change into a nucleotide sequence, the base editing system comprising a napDNAbp and a deaminase, the method comprising: using software executing on at least one computer hardware processor to perform: obtaining input data indicative of the nucleotide sequence and a set of one or more guide RNAs; generating first input features from the input data; applying a first machine learning model to the first input features to obtain first output data indicative, for each one guide RNA in the set of guide RNAs, of bystander editing activity, at one or multiple locations in the nucleotide sequence, by the base editing system when using the each one guide RNA; and identifying, using the first output data, the guide RNA for use in the base editing system for introducing the target change into the nucleotide sequence.

Such a method may further comprise an efficiency machine learning model, comprising generating second input features from the input data; applying a second machine learning model to the second input features to obtain second output data indicative, for each one guide RNA in the set of guide RNAs, of a base editing efficiency, at one or multiple locations in the nucleotide sequence, of the base editing system when using the each one guide RNA, wherein identifying the guide RNA is performed using the first output data and the second output data.

In other aspects, the disclosure provides at least one computer readable storage medium storing processor executable instructions that, when executed by at least one computer hardware processor, cause the at least one computer hardware processor to perform a method of identifying a guide RNA for use in a base editing system for introducing a target change into a nucleotide sequence, the base editing system comprising a napDNAbp and a deaminase, the method comprising: obtaining input data indicative of the nucleotide sequence and a set of one or more guide RNAs; generating first input features from the input data; applying a first machine learning model to the first input features to obtain first output data indicative, for each one guide RNA in the set of guide RNAs, of bystander editing activity, at one or multiple locations in the nucleotide sequence, by the base editing system when using the each one guide RNA; and identifying, using the first output data, the guide RNA for use in the base editing system for introducing the target change into the nucleotide sequence.

In still other aspects, the disclosure provides a system, comprising: at least one computer hardware processor; and at least one computer readable storage medium storing processor executable instructions that, when executed by at least one computer hardware processor, cause the at least one computer hardware processor to perform a method of identifying a guide RNA for use in a base editing system for introducing a target change into a nucleotide sequence, the base editing system comprising a napDNAbp and a deaminase, the method comprising: obtaining input data indicative of the nucleotide sequence and a set of one or more guide RNAs; generating first input features from the input data; applying a first machine learning model to the first input features to obtain first output data indicative, for each one guide RNA in the set of guide RNAs, of bystander editing activity, at one or multiple locations in the nucleotide sequence, by the base editing system when using the each one guide RNA; and identifying, using the first output data, the guide RNA for use in the base editing system for introducing the target change into the nucleotide sequence.

In other aspects, the disclosure provides a method, comprising: using software executing on at least one computer hardware processor to perform: receiving input data indicative of a selection of: a nucleotide sequence; a base editing system comprising a napDNAbp and a deaminase; and a first guide RNA; applying a first machine learning model to the first input features, generated from the input data, to obtain first output data indicative of a base editing efficiency, at a target location in the nucleotide sequence, of the base editing system when using the first guide RNA; applying a second machine learning model to the second input features, generated from the input data, to obtain second output data indicative of bystander editing activity, at one or multiple locations in the nucleotide sequence, by the base editing system when using the first guide RNA; and determining, using the first output data and the second output data, a likelihood of whether the first guide RNA and the base editing system, when used in combination, will result in introduce a target change to the nucleotide sequence in a cell.

The disclosure also provides at least one computer-readable storage medium storing processor-executable instructions that, when executed by at least one computer hardware processor, cause the at least one computer processor to perform: receiving input data indicative of a selection of: a nucleotide sequence; a base editing system comprising a napDNAbp and a deaminase; and a first guide RNA; applying a first machine learning model to the first input features, generated from the input data, to obtain first output data indicative of a base editing efficiency, at a target location in the nucleotide sequence, of the base editing system when using the first guide RNA; applying a second machine learning model to the second input features, generated from the input data, to obtain second output data indicative of bystander editing activity, at one or multiple locations in the nucleotide sequence, by the base editing system when using the first guide RNA; and determining, using the first output data and the second output data, a likelihood of whether the first guide RNA and the base editing system, when used in combination, will result in introduce a target change to the nucleotide sequence in a cell.

In other aspects, the disclosure provides a system, comprising: at least one computer hardware processor; and at least one computer-readable storage medium storing processor-executable instructions that, when executed by the at least one computer hardware processor, cause the at least one computer processor to perform: receiving input data indicative of a selection of: a nucleotide sequence; a base editing system comprising a napDNAbp and a deaminase; and a first guide RNA; applying a first machine learning model to the first input features, generated from the input data, to obtain first output data indicative of a base editing efficiency, at a target location in the nucleotide sequence, of the base editing system when using the first guide RNA; applying a second machine learning model to the second input features, generated from the input data, to obtain second output data indicative of bystander editing activity, at one or multiple locations in the nucleotide sequence, by the base editing system when using the first guide RNA; and determining, using the first output data and the second output data, a likelihood of whether the first guide RNA and the base editing system, when used in combination, will result in introduce a target change to the nucleotide sequence in a cell.

Accordingly, the present disclosure relates, at least to, but not limited by, the following numbered aspects:
1. A computational method of selecting a guide RNA for use in a base editing system comprising a napDNAbp and a deaminase, said base editing system being capable of introducing a genetic change into a nucleotide sequence of a target genomic location to achieve a goal genotype outcome, the method comprising:
   (a) accessing first data indicative of:
      the goal genotype outcome; and
      a plurality of sets of candidate base editing determinates;
   (b) processing the first data using a first computational model to determine second data indicative of a base editing efficiency at the target genomic location for each set of candidate base editing determinates;
   (c) processing the first data using a second computational model to determine third data indicative of a bystander precision for each set of candidate base editing determinates; and
   (d) analyzing the second data and third data to identify a guide RNA capable of achieving the goal genotype outcome.
2. The computational method of aspect 1, wherein the base editing system comprises a base editor that comprises a fusion protein.
3. The computational method of aspect 2, wherein the fusion protein comprises a nucleic acid programmable DNA binding protein (napDNAbp) coupled to a deaminase.
4. The computational method of aspect 3, wherein the deaminase is a cytidine deaminase.
5. The computational method of aspect 3, wherein the deaminase is a adenosine deaminase.
6. The computational method of aspect 4, wherein the cytidine deaminase comprises an amino acid sequence selected from the group consisting of: SEQ ID NOs: 92-134, or a polypeptide having an amino acid sequence having at least 85% sequence identity with SEQ ID NOs: 92-134.
7. The computational method of aspect 5, wherein the adenosine deaminase comprises an amino acid sequence selected from the group consisting of: SEQ ID NOs: 78-91, or a polypeptide having an amino acid sequence having at least 85% sequence identity with SEQ ID NOs: 78-91.
8. The computational method of aspect 3, wherein the napDNAbp is a Cas9 domain.
9. The computational method of aspect 8, wherein the Cas9 domain comprises an amino acid sequence selected from the group consisting of: SEQ ID NOs: 5, 8, 10, 12, and 13-77, or a polypeptide having an amino acid sequence having at least 85% sequence identity with SEQ ID NOs: 5, 8, 10, 12, and 13-77.
10. The computational method of aspect 2, wherein the fusion protein comprises an amino acid sequence selected from the group consisting of: SEQ ID NOs: 174-222, 463-476, or 223-248, or a polypeptide having an amino acid sequence having at least 85% sequence identity with SEQ ID NOs: 174-222, 463-476, or 223-248.
11. The computational method of aspect 1, wherein the base editing determinates comprise one or more of:
   (i) the choice of the napDNAbp of the base editing system;
   (ii) the choice of the deaminase of the base editing system;
   (iii) the nucleotide sequence;
   (iv) the target genomic location;
   (v) the transcriptional state of the target genomic location;
   (vi) locus-dependent activity of the choice napDNAbp;
   (vii) cell-type;
   (viii) transcriptional state of DNA repair proteins; or
   (ix) base editor modifications.
12. The method of aspect 1, wherein the genetic change is to a genetic mutation.
13. The method of aspect 12, wherein the genetic mutation is a single-nucleotide polymorphism, a deletion mutation, an insertion mutation, or a microduplication error.
14. The method of aspect 12, wherein the genetic mutation causes a disease or a risk of a disease.
15. The method of aspect 14, wherein the disease is a monogenic disease.
16. The method of aspect 15, wherein the monogenic disease is sickle cell disease, cystic fibrosis, polycystic kidney disease, Tay-Sachs disease, achondroplasia, beta-thalassemia, Hurler syndrome, severe combined immunodeficiency, hemophilia, glycogen storage disease Ia, and Duchenne muscular dystrophy.
17. The method of aspect 1, wherein the first and second computational models are deep learning computational models.
18. The method of aspect 1, wherein the first and second computational models are neural network models having one or more hidden layers.
19. The method of aspect 1, wherein the computational model is trained with experimental base editing data.
20. A method of introducing a goal genotype outcome in the genome of a cell with a desired base editing system comprising:
   (i) selecting a guide RNA for use in the desired base editing system in accordance with the method of any of aspects 1-19; and
   (ii) contacting the genome of the cell with the guide RNA and the desired base editing system, thereby introducing the goal genotype outcome.
21. The method of aspect 20, wherein the method is conducted ex vivo, in vivo, or ex vivo.
22. The method of aspect 1, wherein the goal genotype outcome restores the function of a gene.
23. The method of aspect 1, wherein the goal genotype outcome restores the function of a disease-causing mutation.
24. A library for training the computational method of aspect 1, comprising a plurality of vectors each comprising a first nucleotide sequence of a target genomic location having a target site to be edited, and a second nucleotide sequence encoding a cognate guide RNA capable of directing the base editing system to carry out base editing at the target genomic location to achieve the goal genotype outcome.
25. A method for training a computational model of any of aspects 1-23, comprising: (i) preparing a library comprising a plurality of nucleic acid molecules each encoding a nucleotide target sequence and a cognate guide RNA; (ii) introducing the library into a plurality of host cells; (iii) contacting the library in the host cells with a Cas-based genome editing system to produce a plurality of genomic repair products; (iv) determining the sequences of the genomic repair products; and (iv) training the computational model with input data that comprises at least the sequences of the genomic repair products and the cognate guide RNA.

I. Machine Learning Algorithm for Base Editing (BE-Hive)

The present disclosure provides a novel machine learning algorithm capable of assisting those of ordinary skill in the art to conduct base editing by, inter alia, facilitating the selection of an appropriate guide RNA and base editor combination which are capable of conducting base editing at a certain level of efficiency and specificity on a given input target DNA sequence desired to be edited to produce an outcome genotype of interest. The novel machine learning algorithm described and claimed herein can be referred to as “BE-Hive.” The disclosure further provides a graphical user interface that implements BE-Hive, allowing a user to input various features, including a desired target DNA sequence, an appropriate guide RNA (or associated CRISPR protospacer), a base editor, and a cell in which base editing is to take place, and to predict base editing efficiencies and bystander editing patterns for the selected features.

In various aspects, the instant specification describes machine learning algorithms for selecting guide RNAs for base editing based on a particular base editor and other determinants of base editing, which include, but are not limited to the choice of the napDNAbp of the base editing system; the choice of the deaminase of the base editing system; the nucleotide sequence; the target genomic location; the transcriptional state of the target genomic location; locus-dependent activity of the choice napDNAbp; cell-type; transcriptional state of DNA repair proteins; and base editor modifications. The disclosure also provides machine learning algorithms for predicting genotype outcomes based on a particular base editor and other determinants of base editing, which include, but are not limited to the choice of the napDNAbp of the base editing system; the choice of the deaminase of the base editing system; the nucleotide sequence; the target genomic location; the transcriptional state of the target genomic location; locus-dependent activity of the choice napDNAbp; cell-type; transcriptional state of DNA repair proteins; and base editor modifications. The disclosure further provides base editors (e.g., ABEs and CBEs), napDNAbps, cytidine deaminases, adenosine deaminases, nucleic acid sequences encoding base editors and components thereof, vectors, and cells. In addition, the disclosure provides methods of making biological or experimental training and/or validation data for training and/or validating the machine learning computational models, as well as, vectors, libraries, and nucleic acid sequences for use in obtaining said experimental training and/or validation data, as well as the experimental training data and/or validation data itself.

The machine learning algorithm considers various inputs, including the sequence of the target DNA sequence to be edited, the napDNAbp options, the deaminase options, the guide RNA options, the spacer and/or protospacer sequence associated with the RNA options, dinucleotide composition at neighboring positions in the protospacers, guide RNA melting temperatures, and the total number of G, C, A, and/or T nucleotides in the protospacer sequence, among other features. In addition, other features that may be considered as input to the machine learning algorithm. Such features may include, but are not limited to, the transcriptional state of the target genomic location, cell-type in which the base editing is taking place, transcriptional state of the target DNA being edited, and any epigenetic modifications of the target DNA being edited.

The disclosure further provides base editors (e.g., ABEs and CBEs), napDNAbps, cytidine deaminases, adenosine deaminases, nucleic acid sequences encoding base editors and/or guide RNAs, vectors, and cells. In other aspects, the disclosure provides guide RNA sequences (and/or spacer sequences or protospacer sequences associated therewith) that can be selected and/or identified by the machine learning algorithm described herein, as well as compositions comprising said guide RNA sequences and a base editor for editing a target DNA sequence (e.g., correcting a point mutation). In addition, the disclosure provides methods of making biological or experimental training and/or validation data for training and/or validating the machine learning algorithms described herein, as well as, vectors, libraries, and nucleic acid sequences for use in obtaining said experimental training and/or validation data, as well as the experimental training data and/or validation data itself.

In one aspect, the disclosure provides a method of using at least one machine learning model to identify a guide RNA for use in a base editing system for introducing a target change into a nucleotide sequence, the base editing system comprising a napDNAbp and a deaminase, the method comprising: using software executing on at least one computer hardware processor to perform: obtaining input data indicative of the nucleotide sequence and a set of one or more guide RNAs; generating first input features from the input data; applying a first machine learning model to the first input features to obtain first output data indicative, for each one guide RNA in the set of guide RNAs, of a base editing efficiency, at one or multiple locations in the nucleotide sequence, of the base editing system when using the each one guide RNA; generating second input features from the input data; applying a second machine learning model to the second input features to obtain second output data indicative, for each one guide RNA in the set of guide RNAs, of bystander editing activity, at one or multiple locations in the nucleotide sequence, by the base editing system when using the each one guide RNA; and identifying, using the first output data and the second output data, the guide RNA for use in the base editing system for introducing the target change into the nucleotide sequence.

In certain embodiments, the set of guide RNAs includes a first guide RNA, and wherein, the input data includes first data indicative of at least a part of a nucleotide sequence associated with the first guide RNA.

The first data can specify a spacer or a protospacer sequence associated with the first guide RNA.

The step of obtaining the data indicative of the nucleotide sequence and the set of guide RNAs, can comprise: obtaining, by the software and from at least one source external to the software, the data indicative of the nucleotide sequence and the set of guide RNAs.

The step of obtaining the data indicative of the nucleotide sequence and the set of guide RNAs, comprises: obtaining, by the software and from at least one source external to the software, first data indicative of the nucleotide sequence; and generating, from the first data indicative of the nucleotide sequence, data indicative of the set of guide RNAs.

In certain embodiments, the first machine learning model comprises a non-linear machine learning model selected from the group consisting of a random forest model, a logistic regression model, a support vector machine model, a generalized linear model, a hierarchical Bayesian model, and neural network model.

In other embodiments, the first machine learning model can comprise a random forest model.

The set of guide RNAs can include a first guide RNA, and wherein generating the first input features comprises generating multiple features to include in the first input features, the multiple features including: features encoding at least some nucleotides in a protospacer sequence or spacer sequence associated with the first guide RNA; and features encoding at least some nucleotides, in the nucleotide sequence, located within a threshold number of nucleotides of the protospacer sequence associated with the first guide RNA.

The step of generating the features encoding the at least some nucleotides in the protospacer sequence comprises generating a one-hot encoding of the at least some nucleotides in the protospacer sequence.

In various embodiments, the multiple features further include one or more of the following features: features encoding at least some dinucleotides at neighboring positions in the protospacer sequence; features representing melting temperature of the first guide RNA; one or more features representing a total number of G, C, A, and/or T nucleotides in the protospacer sequence; and a feature representing an average base editing efficiency of the base editing system.

In certain embodiments, the set of guide RNAs includes a first guide RNA, wherein the first output data is indicative of a fraction of sequence reads containing at least one base edit at any nucleotide in a target window about a protospacer sequence associated with the first guide RNA, among all sequence reads.

In other embodiments, the second first machine learning model comprises a non-linear machine learning model selected from the group consisting of a random forest model, a logistic regression model, a support vector machine model, a generalized linear model, a hierarchical Bayesian model, and neural network model.

In yet other embodiments, the second machine learning model comprises a deep neural network model.

The neural network model can comprise a conditional autoregressive neural network model.

The conditional autoregressive neural network model can include: an encoder neural network mapping input data to a latent representation; and a decoder neural network mapping the latent representation to output data, wherein the decoder neural network has an autoregressive structure.

The encoder neural network can comprise a multi-layer fully connected network with residual connections.

The decoder neural network can generate a distribution over base editing outcomes at each nucleotide while conditioning on previously-generated outcomes.

The neural network model can include parameters representing a position-wise bias toward producing an unedited outcome.

The set of guide RNAs can include a first guide RNA, and wherein generating the second input features can comprise generating multiple features to include in the second input features, the multiple features including: features encoding at least some nucleotides in a protospacer sequence or spacer sequence associated with the first guide RNA; and features encoding at least some nucleotides, in the nucleotide sequence, located within a threshold number of nucleotides of the protospacer sequence associated with the first guide RNA.

In other embodiments, the second output data can be indicative of frequencies of occurrence of base editing outcomes each of which includes edits to nucleotides at multiple positions.

The second output data can be indicative of a frequency distribution on combinations of base editing outcomes. In various embodiments, the set of guide RNAs can include a first guide RNA, wherein, for a specific combination of base edits, the second output data is indicative of a frequency of occurrence of the specific combination of base edits among all sequenced reads containing at least one base edit at any nucleotide in a target window about a protospacer sequence associated with the first guide RNA.

In other embodiments, the set of guide RNAs can include a first guide RNA, wherein the first output data includes a first base editing efficiency value for the first guide RNA, wherein the second output data includes a first bystander editing value for the first guide RNA, and wherein identifying the guide RNA using the first output data and the second output data, comprises multiplying the first base editing efficiency value by the first bystander editing value.

In certain embodiments, the first machine learning model comprises a first plurality of values for a respective first plurality of parameters, the first plurality of values used by the at least one computer hardware processor to obtain the first output data from the first input features.

The first plurality of parameters can comprise at least one thousand parameters. The first plurality of parameters can comprise between one thousand and ten thousand parameters. In various embodiments, the first machine learning model can comprise a random forest model comprising at least 100 decision trees, each of the at least 100 decision trees having at least a depth of D, and wherein processing the input data using the random forest model comprises performing 100*D comparisons. The random forest model can comprise at least 500 decision trees. In certain embodiments, depth of D can be greater than or equal to five, wherein processing the input data using the random forest model comprises performing at least 2500 comparisons.

In other embodiments, the second machine learning model can comprise a second plurality of values for a respective second plurality of parameters, the second plurality of values used by the at least one computer hardware processor to obtain the second output data from the second input features.

The second plurality of parameters can comprise at least ten thousand parameters, or between 25,000 and 100,000 parameters, or between 30,000 and 40,000 parameters.

In other embodiments, the disclosure provides a method of manufacturing the identified guide RNA for use in the base editing system for introducing the target change into the nucleotide sequence.

In other aspects, the disclosure provides a method for training the first machine learning model of any of the above aspects comprising: (i) preparing a library comprising a plurality of nucleic acid molecules each encoding a nucleotide target sequence and a cognate guide RNA; (ii) introducing the library into a plurality of host cells; (iii) contacting the library in the host cells with a Cas-based genome editing system to produce a plurality of genomic repair products; (iv) determining the sequences of the genomic repair products; and (v) training the first machine learning model with training data that comprises at least the sequences of the genomic repair products and the cognate guide RNA.

In still other embodiments, the disclosure provides a method for training the second machine learning model of any of the above aspects comprising: (i) preparing a library comprising a plurality of nucleic acid molecules each encoding a nucleotide target sequence and a cognate guide RNA; (ii) introducing the library into a plurality of host cells; (iii) contacting the library in the host cells with a Cas-based genome editing system to produce a plurality of genomic repair products; (iv) determining the sequences of the genomic repair products; and (v) training the second machine learning model with training data that comprises at least the sequences of the genomic repair products and the cognate guide RNA.

The disclosure also provides for a computer readable storage medium storing processor executable instructions that, when executed by at least one computer hardware processor, cause the at least one computer hardware processor to perform a method of using at least one machine learning model to identify a guide RNA for use in a base editing system for introducing a target change into a nucleotide sequence, the base editing system comprising a napDNAbp and a deaminase, the method comprising: obtaining input data indicative of the nucleotide sequence and a set of one or more guide RNAs; generating first input features from the input data; applying a first machine learning model to the first input features to obtain first output data indicative, for each one guide RNA in the set of guide RNAs, of a base editing efficiency, at one or multiple locations in the nucleotide sequence, of the base editing system when using the each one guide RNA; generating second input features from the input data; applying a second machine learning model to the second input features to obtain second output data indicative, for each one guide RNA in the set of guide RNAs, of bystander editing activity, at one or multiple locations in the nucleotide sequence, by the base editing system when using the each one guide RNA; and identifying, using the first output data and the second output data, the guide RNA for use in the base editing system for introducing the target change into the nucleotide sequence.

In another aspect, the disclosure provides a system comprising: at least one computer hardware processor; and at least one computer readable storage medium storing processor executable instructions that, when executed by the at least one computer hardware processor, cause the at least one computer hardware processor to perform a method of using at least one machine learning model to identify a guide RNA for use in a base editing system for introducing a target change into a nucleotide sequence, the base editing system comprising a napDNAbp and a deaminase, the method comprising: obtaining input data indicative of the nucleotide sequence and a set of one or more guide RNAs; generating first input features from the input data; applying a first machine learning model to the first input features to obtain first output data indicative, for each one guide RNA in the set of guide RNAs, of a base editing efficiency, at one or multiple locations in the nucleotide sequence, of the base editing system when using the each one guide RNA; generating second input features from the input data; applying a second machine learning model to the second input features to obtain second output data indicative, for each one guide RNA in the set of guide RNAs, of bystander editing activity, at one or multiple locations in the nucleotide sequence, by the base editing system when using the each one guide RNA; and identifying, using the first output data and the second output data, the guide RNA for use in the base editing system for introducing the target change into the nucleotide sequence.

In other aspects, the disclosure provides a method, comprising: using software executing on at least one computer hardware processor to perform: receiving input data indicative of a selection of: a nucleotide sequence; a base editing system comprising a napDNAbp and a deaminase; and a first guide RNA; applying a first machine learning model to the first input features, generated from the input data, to obtain first output data indicative of a base editing efficiency, at a target location in the nucleotide sequence, of the base editing system when using the first guide RNA; applying a second machine learning model to the second input features, generated from the input data, to obtain second output data indicative of bystander editing activity, at one or multiple locations in the nucleotide sequence, by the base editing system when using the first guide RNA; and determining, using the first output data and the second output data, a likelihood of whether the first guide RNA and the base editing system, when used in combination, will result in introduce a target change to the nucleotide sequence in a cell.

The disclosure also provides at least one computer-readable storage medium storing processor-executable instructions that, when executed by at least one computer hardware processor, cause the at least one computer processor to perform: receiving input data indicative of a selection of: a nucleotide sequence; a base editing system comprising a napDNAbp and a deaminase; and a first guide RNA; applying a first machine learning model to the first input features, generated from the input data, to obtain first output data indicative of a base editing efficiency, at a target location in the nucleotide sequence, of the base editing system when using the first guide RNA; applying a second machine learning model to the second input features, generated from the input data, to obtain second output data indicative of bystander editing activity, at one or multiple locations in the nucleotide sequence, by the base editing system when using the first guide RNA; and determining, using the first output data and the second output data, a likelihood of whether the first guide RNA and the base editing system, when used in combination, will result in introduce a target change to the nucleotide sequence in a cell.

In other aspects, the disclosure provides a system, comprising: at least one computer hardware processor; and at least one computer-readable storage medium storing processor-executable instructions that, when executed by the at least one computer hardware processor, cause the at least one computer processor to perform: receiving input data indicative of a selection of: a nucleotide sequence; a base editing system comprising a napDNAbp and a deaminase; and a first guide RNA; applying a first machine learning model to the first input features, generated from the input data, to obtain first output data indicative of a base editing efficiency, at a target location in the nucleotide sequence, of the base editing system when using the first guide RNA; applying a second machine learning model to the second input features, generated from the input data, to obtain second output data indicative of bystander editing activity, at one or multiple locations in the nucleotide sequence, by the base editing system when using the first guide RNA; and determining, using the first output data and the second output data, a likelihood of whether the first guide RNA and the base editing system, when used in combination, will result in introduce a target change to the nucleotide sequence in a cell.

In one aspect, the present disclosure provides a machine learning algorithm capable of assisting those of ordinary skill in the art to conduct base editing by, inter alia, facilitating the selection of an appropriate guide RNA and base editor combination which are capable of conducting base editing at a certain level of efficiency and specificity on a given input target DNA sequence desired to be edited to produce an outcome genotype of interest. The machine learning algorithm considers various inputs, including the sequence of the target DNA sequence to be edited, the napDNAbp options, the deaminase options, the guide RNA options, the spacer and/or protospacer sequence associated with the RNA options, dinucleotide composition at neighboring positions in the protospacers, guide RNA melting temperatures, and the total number of G, C, A, and/or T nucleotides in the protospacer sequence, among other features. In addition, other features that may be considered as input to the machine learning algorithm. Such features may include, but are not limited to, the transcriptional state of the target genomic location, cell-type in which the base editing is taking place, transcriptional state of the target DNA being edited, and any epigenetic modifications of the target DNA being edited.

The disclosure further provides a graphical user interface that implements BE-Hive, allowing a user to input various features, including a desired target DNA sequence, an appropriate guide RNA (or associated CRISPR protospacer), a base editor, and a cell in which base editing is to take place, and to predict base editing efficiencies and bystander editing patterns for the selected features.

In certain embodiments, the disclosure provides machine learning computational models for selecting guide RNAs for base editing based on a particular base editor and other determinants of base editing, which include, but are not limited to the choice of the napDNAbp of the base editing system; the choice of the deaminase of the base editing system; the nucleotide sequence; the target genomic location; the transcriptional state of the target genomic location; locus-dependent activity of the choice napDNAbp; cell-type; transcriptional state of DNA repair proteins; and base editor modifications. The disclosure also provides machine learning computational models for predicting genotype outcomes based on a particular base editor and other determinants of base editing, which include, but are not limited to the choice of the napDNAbp of the base editing system; the choice of the deaminase of the base editing system; the nucleotide sequence; the target genomic location; the transcriptional state of the target genomic location; locus-dependent activity of the choice napDNAbp; cell-type; transcriptional state of DNA repair proteins; and base editor modifications. The disclosure further provides base editors (e.g., ABEs and CBEs), napDNAbps, cytidine deaminases, adenosine deaminases, nucleic acid sequences encoding base editors and components thereof, vectors, and cells. In addition, the disclosure provides methods of making biological or experimental training and/or validation data for training and/or validating the machine learning computational models, as well as, vectors, libraries, and nucleic acid sequences for use in obtaining said experimental training and/or validation data, as well as the experimental training data and/or validation data itself.

In one embodiment, the disclosure provides a computational method of selecting a guide RNA for use in a base editing system comprising a napDNAbp and a deaminase, said base editing system being capable of introducing a genetic change into a nucleotide sequence of a target genomic location to achieve a goal genotype outcome, the method comprising: (a) accessing first data indicative of: the goal genotype outcome; and a plurality of sets of candidate base editing determinates; (b) processing the first data using a first computational model to determine second data indicative of a base editing efficiency at the target genomic location for each set of candidate base editing determinates; (c) processing the first data using a second computational model to determine third data indicative of a bystander precision for each set of candidate base editing determinates; and (d) analyzing the second data and third data to identify a guide RNA capable of achieving the goal genotype outcome.

In one embodiment, the computational method comprises a (1) base editing efficiency model together with (2) a bystander editing model.

### Base Editing Efficiency Model

The machine learning algorithm described herein (e.g., BE-Hive) can comprise a base efficiency machine learning model.

Base editing efficiency varies by experimental batch. To combine replicates across batches, first a mean centering and logit transformation was performed at up to 10,638 gRNA-target pairs in each experimental condition separately from the 12kChar library which includes all 4-mers surrounding A or C from protospacer positions 1 to 11. Data was discarded at target sites with fewer than 100 total reads, then averaged values at matched target sites across experimental replicates. Values of negative or positive infinity (resulting from logit of 0 or 1) were discarded. The data were randomly split into training and test sets at a ratio of 90:10. Each target site had a single output value corresponding to the mean logit fraction of sequenced reads with any base editing activity. Data points comprising a single replicate were assigned weight=0.5. Data points comprising multiple replicates were assigned a weight of the median logit variance divided by the logit variance at that data point, or 1, whichever value was smaller. In this manner, exactly half of the data points comprising multiple replicates were assigned a weight of 1, and those with higher variance were assigned a lower weight.

Features from each target sequence were obtained using protospacer positions −9 to 21. Features included one-hot encoded single nucleotide identities at each position, one-hot encoded dinucleotides at neighboring positions, the melting temperature of the sequence and various subsequences, the total number of each nucleotide in the sequence, and the total number of G or C nucleotides in the sequence. Gradient-boosted regression trees from the python package scikit-learn were used, and trained with tuples of (x, y, weights) using the training data. Hyperparameter optimization was performed by varying the number of estimators between {100, 250, 500}, the minimum samples per leaf in {2, 5}, and the maximum tree depth in {2, 3, 4, 5}. A 5-fold cross-validation was performed by splitting the training set into a training and validation set at a ratio of 8:1 and retained the combination of hyperparameters with the strongest average cross-validation performance as the final model. Models were trained in this manner for each combination of cell-type and base editor. Models were evaluated on the test set which was not used during hyperparameter optimization.

In other aspects, the machine learning model can include or be based solely on a base editing efficiency machine learning model, for example, a method identifying a guide RNA for use in a base editing system for introducing a target change into a nucleotide sequence, the base editing system comprising a napDNAbp and a deaminase, the method comprising: using software executing on at least one computer hardware processor to perform: obtaining input data indicative of the nucleotide sequence and a set of one or more guide RNAs; generating first input features from the input data; applying a first machine learning model to the first input features to obtain first output data indicative, for each one guide RNA in the set of guide RNAs, of a base editing efficiency, at one or multiple locations in the nucleotide sequence, of the base editing system when using the each one guide RNA; and identifying, using the first output data, the guide RNA for use in the base editing system for introducing the target change into the nucleotide sequence.

Nevertheless, in such aspects, the machine learning model can further comprise a bystander model, comprising generating second input features from the input data; applying a second machine learning model to the second input features to obtain second output data indicative, for each one guide RNA in the set of guide RNAs, of bystander editing activity, at one or multiple locations in the nucleotide sequence, by the base editing system when using the each one guide RNA, wherein identifying the guide RNA is performed using the first output data and the second output data.

The disclosure also provides at least one computer readable storage medium storing processor executable instructions that, when executed by at least one computer hardware processor, cause the at least one computer hardware processor to perform a method of identifying a guide RNA for use in a base editing system for introducing a target change into a nucleotide sequence, the base editing system comprising a napDNAbp and a deaminase, the method comprising: obtaining input data indicative of the nucleotide sequence and a set of one or more guide RNAs; generating first input features from the input data; applying a first machine learning model to the first input features to obtain first output data indicative, for each one guide RNA in the set of guide RNAs, of a base editing efficiency, at one or multiple locations in the nucleotide sequence, of the base editing system when using the each one guide RNA; and identifying, using the first output data, the guide RNA for use in the base editing system for introducing the target change into the nucleotide sequence.

In other aspects, the disclosure provides a system, comprising: at least one computer hardware processor; and at least one computer readable storage medium storing processor executable instructions that, when executed by the at least one computer hardware processor, cause the at least one computer hardware processor to perform a method of identifying a guide RNA for use in a base editing system for introducing a target change into a nucleotide sequence, the base editing system comprising a napDNAbp and a deaminase, the method comprising: obtaining input data indicative of the nucleotide sequence and a set of one or more guide RNAs; generating first input features from the input data; applying a first machine learning model to the first input features to obtain first output data indicative, for each one guide RNA in the set of guide RNAs, of a base editing efficiency, at one or multiple locations in the nucleotide sequence, of the base editing system when using the each one guide RNA; and identifying, using the first output data, the guide RNA for use in the base editing system for introducing the target change into the nucleotide sequence.

### Bystander Editing Model

The machine learning algorithm described herein (e.g., BE-Hive) can comprise a bystander editing machine learning model.

A dataset was assembled where each gRNA-target pair was matched with a table of observed base editing genotypes and their frequencies among reads with edited outcomes. Data points with fewer than 100 edited reads were discarded. Edited genotypes occurring at higher than 2.5% frequency with no edits at any substrate nucleotides (defined as C for CBEs and A for ABEs) in positions 1-10 were also discarded. Data from multiple experimental replicates were combined by summing read counts for each observed genotype.

Briefly, a deep conditional autoregressive model was designed and implemented that used an input target sequence surrounding a protospacer and PAM to output a frequency distribution on combinations of base editing outcomes in the python package pytorch. The model predicts substitutions at cytosines and guanines for CBEs and adenines and cytosines for ABEs from protospacer positions −10 to 20. The model transforms each substrate nucleotide and its local context using a shared encoder into a deep representation, then applies an autoregressive decoder that iteratively generates a distribution over base editing outcomes at each substrate nucleotide while conditioning on all previous generated outcomes. The encoder and decoder are coupled with a learned position-wise bias towards producing an unedited outcome. The model is trained on observed data by minimizing the KL divergence. Importantly, the conditional autoregressive design is sufficiently expressive to learn any possible joint distribution in the output space, thereby representing a powerful and general method for learning the editing tendencies of any base editor from data.

Input features were obtained by one-hot encoding each substrate nucleotide and the 5 nucleotides (where 5 is a hyperparameter) on either side of it and concatenating this with a one-hot encoding of the position of the substrate nucleotide within positions −9 to 20. Additional features considered but found to detract from model performance during hyperparameter optimization included concatenating a one-hot encoding of the full sequence context. Hyperparameter optimization on the radii of nucleotides surrounding the substrate nucleotide considered values in {3, 5, 7, 9}, and found 5 to be optimal when averaged across hyperparameter optimization rounds that included simultaneous changes in other hyperparameters. Each substrate nucleotide within the editing range were featurized in this manner for each target sequence.

The model uses two neural networks: an encoder with two hidden layers of 64 neurons and a decoder with five hidden layers of 64 neurons. The networks are fully connected, use ReLU activations, and contain residual connections between neighboring pairs of layers that have equal shape. A dropout frequency of 5.0% was used and tuned by hyperparameter optimization. An architecture search in hyperparameter optimization was included and found that these shapes were a local optimum in the surrounding neighborhood varying the number of neurons per layer and the number of layers in each network.

During a forward pass of the model at a single target site, the shapes of relevant variables are:
x.shape=(n.edit.b, x_dim)
y_mask.shape=(n.uniq.e+1, n.edit.b, y_mask_dim)
target.shape=(n.uniq.e+1, n.edit.b, 4, 1)
obs_freq.shape=(n.uniq.e)
   where:
‘x’ is the featurized input
‘y_mask’ is used to provide previously observed outcomes to the decoder while masking future outcomes, in a conditional autoregressive manner
‘target’ is a one-hot encoding of each unique edited genotype
‘obs_freq’ contains the observed frequencies for each edited genotype
n.uniq.e=the number of unique observed edited genotypes for a target site
n.edit.b=the number of editable bases in the target sequence
x_dim=the number of features for a single substrate nucleotide in a single target sequence.

The shape n.uniq.e+1 is used to indicate the inclusion of a row for the wild-type outcome. The model was run on this outcome and the result was used to adjust all predicted probabilities to obtain a denominator equal to 1−p(wild-type).

The tensor ‘y_mask’ was used to provide previously observed outcomes to the decoder while masking future outcomes in a conditional autoregressive fashion. Previously observed unedited nucleotides are encoded as [1/3, 1/3, 1/3], while editable nucleotides are encoded as [0, 0, 0] if unedited, and otherwise are a one-hot encoding of the nucleotide resulting from the base edit. Future nucleotides are encoded as [−1, −1, −1].

The following shape transformations occur during a forward pass.
1. Model encodes x: (n.edit.b, x_dim)→(n.edit.b, x_enc_dim)
2. Expanding and concatenating with y_mask→(n.uniq.e+1, n.edit.b, x_enc_dim+y_mask_dim).
3. Decode→(n.uniq.e+1, n.edit.b, 1, 4)
4. Add unedited bias, then log softmax→(n.uniq.e+1, n.edit.b, 1, 4)
5. Matrix multiplication with target one-hot-encoding→(n.uniq.e+1, n.edit.b, 1, 1), reshape→(n.uniq.e+1, n.edit.b)
6. Sum log likelihoods→(n.uniq.e+1)
7. Adjust all likelihoods by (1−wild-type) denominator→(n.uniq.e). The wild-type outcome is encoded at the last position.

The resulting (n.uniq.e) shape vector contains a number corresponding to the predicted frequency of each unique observed genotype (totaling n.uniq.e). To obtain a loss during training, the KL divergence between the predicted frequency distribution and the observed frequency distribution is used.

A learnable bias toward unedited outcomes is a part of the model. This component uses an input shape of (n.uniq.e+1, n.edit.b, 1, 4) and outputs a tensor with equivalent shape: (n.uniq.e+1, n.edit.b, 1, 4). Its parameters correspond to a single value for each position and substrate nucleotide representing a bias towards producing an unedited outcome. One important aspect of the structure of the data is that most dimensions of the input and output tensors vary by target site. Batches comprised of groups of target sites. Empirically, it was observed that this property caused minimal speed gains when training the model on CPUs vs GPUs.

Thus, in various aspects, the machine learning model can include or be based solely on a bystander machine learning model, comprising a method of identifying a guide RNA for use in a base editing system for introducing a target change into a nucleotide sequence, the base editing system comprising a napDNAbp and a deaminase, the method comprising: using software executing on at least one computer hardware processor to perform: obtaining input data indicative of the nucleotide sequence and a set of one or more guide RNAs; generating first input features from the input data; applying a first machine learning model to the first input features to obtain first output data indicative, for each one guide RNA in the set of guide RNAs, of bystander editing activity, at one or multiple locations in the nucleotide sequence, by the base editing system when using the each one guide RNA; and identifying, using the first output data, the guide RNA for use in the base editing system for introducing the target change into the nucleotide sequence.

Such a method may further comprise an efficiency machine learning model, comprising generating second input features from the input data; applying a second machine learning model to the second input features to obtain second output data indicative, for each one guide RNA in the set of guide RNAs, of a base editing efficiency, at one or multiple locations in the nucleotide sequence, of the base editing system when using the each one guide RNA, wherein identifying the guide RNA is performed using the first output data and the second output data.

In other aspects, the disclosure provides at least one computer readable storage medium storing processor executable instructions that, when executed by at least one computer hardware processor, cause the at least one computer hardware processor to perform a method of identifying a guide RNA for use in a base editing system for introducing a target change into a nucleotide sequence, the base editing system comprising a napDNAbp and a deaminase, the method comprising: obtaining input data indicative of the nucleotide sequence and a set of one or more guide RNAs; generating first input features from the input data; applying a first machine learning model to the first input features to obtain first output data indicative, for each one guide RNA in the set of guide RNAs, of bystander editing activity, at one or multiple locations in the nucleotide sequence, by the base editing system when using the each one guide RNA; and identifying, using the first output data, the guide RNA for use in the base editing system for introducing the target change into the nucleotide sequence.

In still other aspects, the disclosure provides a system, comprising: at least one computer hardware processor; and at least one computer readable storage medium storing processor executable instructions that, when executed by at least one computer hardware processor, cause the at least one computer hardware processor to perform a method of identifying a guide RNA for use in a base editing system for introducing a target change into a nucleotide sequence, the base editing system comprising a napDNAbp and a deaminase, the method comprising: obtaining input data indicative of the nucleotide sequence and a set of one or more guide RNAs; generating first input features from the input data; applying a first machine learning model to the first input features to obtain first output data indicative, for each one guide RNA in the set of guide RNAs, of bystander editing activity, at one or multiple locations in the nucleotide sequence, by the base editing system when using the each one guide RNA; and identifying, using the first output data, the guide RNA for use in the base editing system for introducing the target change into the nucleotide sequence.

Model Training and/or Validation

Various aspects of the disclosure also relate to methods and compositions (e.g., vector libraries, nucleic acid sequences, base editors, guide RNAs, etc.) for generating biological training data (e.g., actual base editing experimental results from a known input target DNA with the output being sequencing data of the resulting genotype post-editing), which can also be used as validation data when in the context of evaluating an already-trained computational model. The following aspects relate to such methods and compositions for training and/or validating the machine learning computational models. Such aspects include library cloning, cloning, cell culture, deep sequencing, and statistical methods.

### (A) Library Cloning

In one embodiment, model training and/or validation involves the preparation of a library of target sequences for contacting with one or more candidate base editors. In one embodiment, library cloning is as reported in Shen et al. 2018, with minor changes. In brief, the process involves ordering a library of 2,000 to 12,000 oligonucleotides pairing an sgRNA protospacer with its 35-nt, 56-nt or 61-nt target site, centered on an NGG or NG PAM, as specified. Pools were amplified with NEBNext Ultra II Q5 Master Mix (New England Biolabs) with initial denaturation and extension times extended to 2 minutes per cycle for all PCR reactions to prevent skewing towards GC-rich sequences. To insert the sgRNA hairpin between the sgRNA protospacer and the target site, the library undergoes an intermediate Gibson Assembly circularization step, restriction enzyme linearization and Gibson Assembly into a plasmid backbone containing a U6 promoter to facilitate sgRNA expression, a hygromycin resistance cassette and flanking Tol2 transposon sites to facilitate integration into the genome. Purified plasmids were transformed into NEB10beta (New England Biolabs) electrocompetent cells. Following recovery, a small dilution series was plated to assess transformation efficiency and the remainder was grown in liquid culture in DRM medium overnight at 37° C. with 100 ug/mL ampicillin. The plasmid library was isolated by Midiprep plasmid purification (Qiagen). Library integrity was verified by restriction digest with SapI (New England Biolabs) for 1 hour at 37° C., and sequence diversity was validated by deep sequencing as described below.

### (B) Cloning

In other embodiments, model training and/or validation involves cloning. Base editor plasmids were constructed by inserting a blasticidin resistance expression cassette from a p2T-CAG-SpCas9-BlastR plasmid (107190) (Arbab et al., 2015) downstream of the bGH-polyA terminator into a BE4 plasmid (100802) (Komor et al., 2017). Tol2-transposase sites from p2T-CAG-SpCas9-BlastR were cloned to flank the base editor and antibiotic selection cassettes. All editors described in this Example were cloned between the N-terminal and C-terminal NLS sequences flanking BE4. The full sequence of the p2T-CAG-BE4max-BlastR plasmid and editor sequences for all editors used in this Example is appended in the ‘Sequences’ section.

Individual SpCas9 sgRNAs were cloned as a pool into a Tol2-transposon-containing gRNA expression plasmid (Addgene 71485) using BbsI plasmid digest and Gibson Assembly (New England Biolabs). Protospacer sequences and gene specific primers used for amplification followed by HTS are listed in the Primers Table.

### (C) Cell Culture

In still other embodiments, model training and/or validation involves cell culture. mESC lines used have been described previously and were cultured as described previously (Sherwood et al., 2014). HEK293T and U20S cells were purchased from ATCC and cultured as recommended by ATCC. Cell lines were authenticated by the suppliers and tested negative for Mycoplasma.

For stable Tol2 transposon library integration, cells were transfected using Lipofectamine 3000 (Thermo Fisher) following standard protocols with equimolar amounts of Tol2 transposase plasmid (a gift from K. Kawakami) and transposon-containing plasmid. For library applications, 15-cm plates with >10⁷ initial cells were used, and for single sgRNA targeting, 48-well plates with >10⁵ initial cells were used. To generate library cell lines with stable Tol2-mediated genomic integration, cells were selected with hygromycin starting the day after transfection at an empirically defined concentration and continued for >2 weeks. In cases where sequential plasmid integration was performed such as integrating library and then base editor, cells were transfected with Tol2 transposase plasmid using Lipofectamine 3000 and selected with blasticidin starting the day after transfection for 4 days before harvesting.

### (D) Deep Sequencing

In yet other embodiments, model training and/or validation involves deep sequence, e.g., sequencing of experimental base editing genotype results. Genomic DNA was collected from cells 5 days after transfection, after 4 days of antibiotic selection. For library samples, 16 μg gDNA was used for each sample; for individual locus samples and untreated cell library samples, 2 μg gDNA was used; for plasmid library verification, 0.5 μg purified plasmid DNA was used. For individual locus samples, the locus surrounding CRISPR-Cas9 mutation was PCR-amplified in two steps using primers >50-bp from the Cas9 target site. PCR1 was performed to amplify the endogenous locus or library cassette using the primers specified below. PCR2 was performed to add full-length Illumina sequencing adapters using the NEBNext Index Primer Sets 1 and 2 (New England Biolabs) or internally ordered primers with equivalent sequences. All PCRs were performed using NEBNext Ultra II Q5 Master Mix. Extension time for all PCR reactions was extended to 2 minutes per cycle to prevent skewing towards GC-rich sequences. Samples were pooled using Tape Station (Agilent) and quantified using a KAPA Library Quantification Kit (KAPA Biosystems). The pooled samples were sequenced using NextSeq or MiSeq (Illumina).

### (E) Library Names

Supplementary figures, tables, and deposited data use different names for designed libraries than the manuscript for convenience. The “comprehensive context library” is referred to as “12kChar” and contains 12,000 target sites designed with all 4-mers surrounding a substrate nucleotide at protospacer positions 1-11 and all 6-mers surrounding an adenine or cytosine at position 6. Three disease-associated libraries called “CBE precision editing SNV library”, “ABE precision editing SNV library”, and “transversion-enriched SNV library” in the manuscript are referred to as “CtoT”, “AtoG”, and “CtoGA”, indicating the base editing event that corrects the disease-related variants included in each library.

### (F) Sequence Motif Models

For prediction tasks where the target variable is continuous and has range in (0, 1), a logistic transformation to the data was applied, and then linear regression was used. For continuous data representing fractions, values equal to 0 or 1 were discarded. For classification tasks, the target variables were either 0 or 1 indicating absence or presence of activity, and logistic regression was used. Target variables included the efficiency of C⋅G-to-T⋅A editing by CBEs, A⋅T-to-G⋅C editing by ABEs, the presence or absence of cytosine editing by ABEs and of guanine editing by CBEs, and the purity of cytosine transversions by CBEs. Each of these statistics involves calculating a denominator corresponding to the total number of reads at a target sequence, or the total number of edited reads at a target sequence. Target sequences with fewer than 100 reads in the denominator were discarded to ensure the accuracy of estimated statistics in the training and testing data. Features were obtained by one-hot-encoding nucleotides per position relative to a substrate nucleotide or to the protospacer. The data were randomly split into training and test sets at an 80:20 ratio. Sequence motifs described by these regression models consider each position independently and are intended primarily for visualization.

### (G) Sequence Alignment and Data Processing

Sequencing reads were assigned to designed library target sites by locality sensitive hashing). Target contexts that were intentionally designed to be highly similar to each other were designed barcodes to assist accurate assignment. Sequence alignment was performed using Smith-Waterman with the parameters: match +1, mismatch −1, indel start −5, indel extend 0. Nucleotides with PHRED score below 30 were assumed to be the reference nucleotide.

For base editing analysis, aligned reads with no indels were retained for analysis and events were defined as the combination of all possible substitutions at all substrate nucleotides in the target site in a read, where a single sequencing read corresponds to an observation of a single event. Substrate nucleotides were defined as C and G for CBEs and A and C for ABEs. For indel analysis, reads containing indels with at least one indel position occurring between protospacer positions −6 to 26 were retained, where position 1 is the 5′-most nucleotide of the protospacer, and 0 is used to refer to the position between −1 and 1. Reads containing indels without at least six nucleotides with at least 90% match frequency on both sides of each indel were discarded. Events were defined as indels identified by position, length, and inserted nucleotides occurring in a read. Combination indels were either not observed at all or only at exceedingly low frequencies in endogenous data and were therefore excluded from consideration when analyzing library data.

### (H) Quantifying Base Editing Profiles

The frequencies of each single-nucleotide mutation were tabulated at each position in each designed target sequence from the sequence alignments. Then, the following steps were applied to adjust treatment data by control data, adjust batch effects and identify base editing mutations that occur at frequencies above background.

The first step was to filter control mutations in control data occurring at or above a 5.0% frequency threshold. As control conditions do not undergo a second selection step (90-95% cell death then expansion), control mutations that are relatively common are highly likely to expand in frequency in treatment data. Since the resulting treatment population frequency (before editing) has high variance (due to the 90-95% cell death then expansion), it is very difficult to de-confound this factor from mutations occurring due to base editing.

The second step was to filter treatment mutations that could be explained by control mutations. The probability of treatment mutations occurring from a binomial distribution parameterized by the observed mutation frequency in the control population and filter mutations was determined at FDR=0.05.

The third step was to filter mutations occurring in both control and treatment conditions, subtract control frequencies from treatment frequencies.

The fourth step was to filter treatment mutations that could be explained by Illumina sequencing errors. The probability of treatment mutations was determined under a binomial distribution parameterized by the lowest quality (>Q30) sequencing call at that position and filter at FDR=0.05. The empirical determined lowest quality is often Q32 or Q36, which correspond to error thresholds of 6e-4 and 2e-4 respectively.

The fifth step was to filter treatment mutations that could be explained by batch effects (comparing treatment vs. treatment). Summary statistics of the mean mutation rate were calculated across all target site with a given substrate nucleotide at a particular position to another nucleotide, yielding an L×12 matrix for each condition, where L=55, 56, or 61. Then, perform one-way ANOVA was performed using the batches defined on the first slide and filter mutations at Bonferroni-corrected p-value threshold of 0.005.

The sixth step was to identify treatment mutations that were consistent by editors across conditions, especially rare ones, while filtering background mutations (comparing treatment vs. treatment). On the batch-effect-corrected L×12 matrix per condition, group by editors, calculate normalized rankings of each mutation within each condition. Perform robust rank aggregation on each mutation to obtain an upper bound on the p-value.

Based on the above analysis, editing profiles were empirically designed for denoising and filtering base editing outcomes. To ensure high sensitivity, these profiles were designed to be broad to minimize the possibility of excluding reads with legitimate base editing activity. For CBEs, base editing activity was defined as C to A, G, or T at positions −9 to 20 and G to A or C at positions −9 to 5. For ABEs, base editing activity was defined as A to G at positions −5 to 20, A to C or T at positions 1 to 10, and C to G or T at positions 1 to 10. For all analysis described herein that required tabulating reads with base editing activity, reads were discarded that did not have base editing activity according to these broad profiles.

(I) Selection of Variants from Disease Databases

Disease variants were selected from the NCBI ClinVar database and the Human Gene Mutation Database (HGMD) for computational screening and subsequent experimental correction using versions of both database that were up to date as of September of 2018. Variants from ClinVar that were designated by at least one lab as ‘pathogenic’ or ‘likely pathogenic’ were retained. Variants from HGMD with a disease association of ‘DM’ or disease-causing mutation were retained.

SpCas9 gRNAs were enumerated for each disease allele. Using a previous version of BE-Hive, predicted correction precisions were predicted for each gRNA-allele combination and used to prioritize the design of libraries. Two libraries of 12,000 gRNA-target pairs were designed called ‘AtoG’ and ‘CtoT’. The ‘AtoG’ library contained 11,585 unique pathogenic variants while ‘CtoT’ contained 7,444 unique pathogenic variants. A third library ‘CtoGA’ with 3,800 gRNA-target pairs targeting pathogenic variants was designed with 2,668 unique pathogenic variants.

### (J) Quantifying the Ratio of Base Editing to Indel Activity

Target sites with greater than 1000 reads and with at least one indel read were retained (to avoid division by zero). Notably, no pseudocounts were used. To calculate BE:indel ratios, library target sites without a substrate nucleotide within the typical base editing window were filtered. These target sites resulted from the library design choices that prioritized diversity and exploration, but these target sites are unlikely to be selected for editing in common user applications. The geometric mean was selected as a summary statistic because BE:indel ratios were distributed roughly log-normal, and the statistic summarizes more of the data than the median.

### (K) Adjusting for Noise in 1-Bp Indels

To characterize rare indels from base editing outcomes, endogenous data (with large sequencing depth, in HEK293T cells) was used and designed certain library conditions were designed (with high editing efficiency and deep sequencing coverage) as gold standards to denoise the other library datasets. In both endogenous data and gold-standard library conditions, the fraction of 1-bp indels was observed to be 5-30% of all indels. In contrast, in many treatment library conditions, the fraction was as high as 80-95%, similar to those in untreated library controls. In addition, these background 1-bp indels appeared to occur nearly uniformly across the target site, while in the “gold standard” conditions, 1-bp indels are concentrated near the HNH nick and typical base editing window. Based on these sets of observations, it was reasoned that the conservative adjustment of treatment conditions by control conditions (by subtracting the frequency of indels at matching target sites, with matching indel start position and length) did not completely adjust noise from treatment data. To enable a more accurate calculation of base editing to indel ratios, an additional quality control step was applied where the frequencies of 1-bp indels in library target sites were decreased uniformly such that the global (across the entire library of sequence contexts) frequency of 1-bp indels was at most 30% of all indels.

### (L) Adjusting for Batch Effects in Base Editing to Indel Ratios

Some batch effects in calculated BE:indel ratios were observed. To adjust for batch effects, two-way ANOVA was applied, crossing experimental batch with base editor, on the geometric mean BE:indel ratio for all library experiments. As instructed by the experimental protocol, the batch must be distinct for each combination of cell-type and library. For this analysis, all point mutants of base editors were dinned with their wild-type versions since small differences in BE:indel ratios were observed that were dominated by differences by experimental batch and by base editor. The average coefficient across all experimental batches was added to the learned coefficient for each base editor to obtain a batch-adjusted coefficient for each base editor. An adjustment factor was obtained as the difference between the average geometric mean BE:indel ratio across experiments for a given base editor and the batch-adjusted coefficient for that base editor. Adjustment factors were used to adjust the BE:indel ratio at individual target sites for analysis requiring such resolution.

### (M) Definition of Disequilibrium Score

Disequilibrium scores are calculated for a given pair of substrate nucleotides as the ratio between the observed joint editing probability and the probability of both nucleotides being edited together assuming statistical independence. Calculating a valid log disequilibrium score from observed data requires non-zero frequencies for p(first nucleotide is edited), p(second nucleotide is edited), and p(first and second nucleotide are edited). Disequilibrium score values above one indicate a tendency for both or neither to be edited together (positive log disequilibrium score), while values below one indicate a tendency for only one or the other to be edited (negative log disequilibrium score).

### (N) Data and Code Availability

The sequencing data generated herein are available at the NCBI Sequence Read Archive database under PRJNA591007. Processed data have been deposited under the following DOIs: 10.6084/m9.figshare.10673816 and 10.6084/m9.figshare.10678097. The code used for data processing and analysis are available at github.com/maxwshen/lib-dataprocessing and github.com/maxwshen/lib-analysis.

### BE-Hive Graphical User Interface (GUI)

In other aspects, the disclosure further provides a graphical user interface that implements BE-Hive, allowing a user to input various features, including a desired target DNA sequence, an appropriate guide RNA (or associated CRISPR protospacer), a base editor, and a cell in which base editing is to take place, and to predict base editing efficiencies and bystander editing patterns for the selected features.

The GUI is available at www.crisprbehive.desing, the contents of which are incorporated herein by reference. In addition, exemplary screen shots of the GUI are provided in FIGS. 24A-24J and explained herein in the Brief Description of the Drawings. As outlined on the above web site, which is incorporated by reference, BE-Hive predicts base editing efficiency and bystander editing patterns for various base editors using machine learning models trained on observed base editing outcomes from up to 10,638 sgRNA-target sequence pairs integrated into the genomes of mouse embryonic stem cells and human HEK293T cells using SpCas9 and Cas9-NG base editors. These sgRNA-target pairs were designed to be minimally biased and maximally cover possible sequence space. Models for different base editors and cell-types were trained separately.

The input to a BE-Hive model is a genomic target sequence and an sgRNA sequence. The user selects which base editor and cell-type, which selects which machine learning models to use.

The editing efficiency model predicts the Z-score relative to the “average” sgRNA-target pair (across our dataset of highly diverse sgRNA-target pairs that cover sequence space with minimal bias). These Z-scores can be converted to the fraction of sequenced reads that have any base editing activity at any nucleotide in the base editing window among all sequenced reads, including unedited wild-type sequenced reads. (See calibration section below). The bystander editing model predicts the frequency of a specific combination of base editing outcomes across all nucleotides in the base editing window among all sequenced reads that have any base editing activity at any nucleotide in the base editing window. The single mode outputs predictions using the above units.

Predictions from the two models can be combined by simple multiplication since the units in the bystander editing model's denominator and the editing efficiency model's numerator are the same. The units of the combined prediction are the frequency of a specific combination of base editing outcomes across all nucleotides in the base editing window among all sequenced reads, including unedited wild-type sequenced reads. Our batch mode combines predictions in this manner when the toggle “Report frequencies among: sequenced reads by including efficiency” is on.

5′G sgRNA Design

The base editing data used for training the models can add a 5′G to a 20-nt protospacer when the first nucleotide is not a G.

We have observed that the base editing window changes depending on whether the protospacer is 20 nt or 21 nt and if the added 5′G is a match or mismatch to the genome. Specifically, when a 21 nt protospacer is used and the 5′G does match the genome, the base editing window is shifted by about 0.5 nucleotides 5′ relative to the window with a 20-nt protospacer.

The BE-Hive models have automatically learned these properties from the training data. If an sgRNA without a 5′G is used where the design rule would otherwise add it, and it would match the genome, it should noted that your base editing window will be shifted 3′ by about 0.5 nucleotides relative to the BE-Hive predictions.

It is possible to artificially adjust for this behavior in a manner that can make BE-Hive predictions slightly more accurate for an application. Specifically, if protospacer position 1 is not a G, and the design rule would prepend a 5′G but it is desired not to, and protospacer position 0 is a G, then one can change the G0 to another nucleotide to effectively “trick” the models into using a 20-nt protospacer. It is recommended not to change G0 to a base editing substrate nucleotide and avoiding strong motifs such as TC for CBEs. With these suggestions in mind, it would be typical to use A0 for CBEs and C0 for ABEs.

### Calibrating Editing Efficiency Predictions

Base editing efficiency depends on cell-type, delivery strategy, and other conditions unique to each experiment. To account for these factors, our base editing efficiency model outputs Z-scores by default, and allows users to provide experiment-specific information to convert the Z-score predictions to the units of the fraction of sequenced reads that have any base editing activity at any nucleotide in the base editing window among all sequenced reads, including unedited wild-type sequenced reads.

The simplest strategy is to provide the “average” editing efficiency observed in your experimental system, where the average is taken over the theoretical set of all sgRNA-target pairs with all possible sequence contexts. Since most base editing experiments avoid sequence contexts known to have poor efficiency (such as those without centrally located cytosines when using cytosine base editors), simply averaging your previous base editing data is likely to overestimate this quantity.

### What is Total Predicted Probability?

In tables of predictions provided by the bystander editing model, there is a column called total predicted probability.

The set of all possible combinations of editing outcomes grows exponentially with the number of substrate nucleotides in a base editing window (denote this as N). At a first glance, this number may appear to be 2\hat{ }N when considering only two possibilities: that each single nucleotide is either edited or not (C or T, in the case of cytosine base editors). However, our work identifies uncommon and rare base editing outcomes including C→G, C→A, G→A conversions by CBEs. Thus when considering cytosine base editing, the possibility space scales as 4\hat{ }N. When N is large, it can take a prohibitive number of forward model evaluations to predict the probability of all exponentially many editing combinations, which would sum to 1.

However, it is known that some editing combinations are more likely than others. To provide predictions in an efficient and expedient manner, greedy heuristics were used to minimize the number of forward model evaluations while maximizing the total probability accounted for. Since we only query the model on a subset of all possible sequences, the total probability observed must be less than 1.

In typical cases, the total predicted probability is 0.95 or greater. For downstream applications, the conservative assumption that the remaining probability are allocated to the least desirable editing outcome possible is recommended.

How is BE-Hive Used with Other Cell-Types?

It is anticipated that base editing activity is generally similar across mammalian cell-types that share similar DNA repair systems. Selecting between mES and HEK293T models by similarity of DNA repair systems is recommended.

How is BE-Hive Used with Other Cas Variants?

The web app does not explicitly filter protospacers by PAM. If the selected Cas variant has similar base editing activity as SpCas9 or Cas9-NG base editors, but has a different PAM, the appropriate protospacers can be selected from the drop-down menus in the web app.

If the Cas variant base editor has different activity than SpCas9 or Cas9-NG base editors, including SaCas9 and Cas12a (Cpf1), please refer to our manuscript and supplementary information which discuss using BE-Hive trained on SpCas9/Cas9-NG base editing data on these Cas variants. The base editing window tends to shift and sometimes widen or narrow when modifying the Cas variant, but deaminase-specific sequence preferences do not change substantially (as one would expect).

II. napDNAbp (Cas9 Domains)

In one aspect, the methods and base editor compositions described herein involve a nucleic acid programmable DNA binding protein (napDNAbp). Each napDNAbp is associated with at least one guide nucleic acid (e.g., guide RNA), which localizes the napDNAbp to a DNA sequence that comprises a DNA strand (i.e., a target strand) that is complementary to the guide nucleic acid, or a portion thereof (e.g., the protospacer of a guide RNA). In other words, the guide nucleic-acid “programs” the napDNAbp (e.g., Cas9 or equivalent) to localize and bind to a complementary sequence. In various embodiments, the napDNAbp can be fused to a herein disclosed adenosine deaminase or cytidine deaminase.

Without being bound by any particular theory, the binding mechanism of a napDNAbp-guide RNA complex, in general, includes the step of forming an R-loop whereby the napDNAbp induces the unwinding of a double-strand DNA target, thereby separating the strands in the region bound by the napDNAbp. The guide RNA protospacer then hybridizes to the “target strand.” This displaces a “non-target strand” that is complementary to the target strand, which forms the single strand region of the R-loop. In some embodiments, the napDNAbp includes one or more nuclease activities, which then cut the DNA leaving various types of lesions. For example, the napDNAbp may comprises a nuclease activity that cuts the non-target strand at a first location, and/or cuts the target strand at a second location. Depending on the nuclease activity, the target DNA can be cut to form a “double-stranded break” whereby both strands are cut. In other embodiments, the target DNA can be cut at only a single site, i.e., the DNA is “nicked” on one strand. Exemplary napDNAbp with different nuclease activities include “Cas9 nickase” (“nCas9”) and a deactivated Cas9 having no nuclease activities (“dead Cas9” or “dCas9”).

The below description of various napDNAbps which can be used in connection with the presently disclose base editors is not meant to be limiting in any way. The base editors may comprise the canonical SpCas9, or any ortholog Cas9 protein, or any variant Cas9 protein—including any naturally occurring variant, mutant, or otherwise engineered version of Cas9—that is known or which can be made or evolved through a directed evolutionary or otherwise mutagenic process. In various embodiments, the Cas9 or Cas9 variants have a nickase activity, i.e., only cleave of strand of the target DNA sequence. In other embodiments, the Cas9 or Cas9 variants have inactive nucleases, i.e., are “dead” Cas9 proteins. Other variant Cas9 proteins that may be used are those having a smaller molecular weight than the canonical SpCas9 (e.g., for easier delivery) or having modified or rearranged primary amino acid structure (e.g., the circular permutant formats). The base editors described herein may also comprise Cas9 equivalents, including Cas12a/Cpf1 and Cas12b proteins which are the result of convergent evolution. The napDNAbps used herein (e.g., SpCas9, Cas9 variant, or Cas9 equivalents) may also contain various modifications that alter/enhance their PAM specifities. Lastly, the application contemplates any Cas9, Cas9 variant, or Cas9 equivalent which has at least 70%, at least 75%, at least 80%, at least 85%, at least 90%, at least 91%, at least 92%, at least 93%, at least 94%, at least 95%, at least 96%, at least 97%, at least 98%, at least 99%, or at least 99.9% sequence identity to a reference Cas9 sequence, such as a references SpCas9 canonical sequence or a reference Cas9 equivalent (e.g., Cas12a/Cpf1).

The napDNAbp can be a CRISPR (clustered regularly interspaced short palindromic repeat)-associated nuclease. As outlined above, CRISPR is an adaptive immune system that provides protection against mobile genetic elements (viruses, transposable elements and conjugative plasmids). CRISPR clusters contain spacers, sequences complementary to antecedent mobile elements, and target invading nucleic acids. CRISPR clusters are transcribed and processed into CRISPR RNA (crRNA). In type II CRISPR systems correct processing of pre-crRNA requires a trans-encoded small RNA (tracrRNA), endogenous ribonuclease 3 (rnc) and a Cas9 protein. The tracrRNA serves as a guide for ribonuclease 3-aided processing of pre-crRNA. Subsequently, Cas9/crRNA/tracrRNA endonucleolytically cleaves linear or circular dsDNA target complementary to the spacer. The target strand not complementary to crRNA is first cut endonucleolytically, then trimmed 3′-5′ exonucleolytically. In nature, DNA-binding and cleavage typically requires protein and both RNAs. However, single guide RNAs (“sgRNA”, or simply “gNRA”) can be engineered so as to incorporate aspects of both the crRNA and tracrRNA into a single RNA species. See, e.g., Jinek M. et al., Science 337:816-821(2012), the entire contents of which is hereby incorporated by reference.

In some embodiments, the napDNAbp directs cleavage of one or both strands at the location of a target sequence, such as within the target sequence and/or within the complement of the target sequence. In some embodiments, the napDNAbp directs cleavage of one or both strands within about 1, 2, 3, 4, 5, 6, 7, 8, 9, 10, 15, 20, 25, 50, 100, 200, 500, or more base pairs from the first or last nucleotide of a target sequence. In some embodiments, a vector encodes a napDNAbp that is mutated to with respect to a corresponding wild-type enzyme such that the mutated napDNAbp lacks the ability to cleave one or both strands of a target polynucleotide containing a target sequence. For example, an aspartate-to-alanine substitution (D10A) in the RuvC I catalytic domain of Cas9 from S. pyogenes converts Cas9 from a nuclease that cleaves both strands to a nickase (cleaves a single strand). Other examples of mutations that render Cas9 a nickase include, without limitation, H840A, N854A, and N863A in reference to the canonical SpCas9 sequence, or to equivalent amino acid positions in other Cas9 variants or Cas9 equivalents.

As used herein, the term “Cas protein” refers to a full-length Cas protein obtained from nature, a recombinant Cas protein having a sequences that differs from a naturally occurring Cas protein, or any fragment of a Cas protein that nevertheless retains all or a significant amount of the requisite basic functions needed for the disclosed methods, i.e., (i) possession of nucleic-acid programmable binding of the Cas protein to a target DNA, and (ii) ability to nick the target DNA sequence on one strand. The Cas proteins contemplated herein embrace CRISPR Cas 9 proteins, as well as Cas9 equivalents, variants (e.g., Cas9 nickase (nCas9) or nuclease inactive Cas9 (dCas9)) homologs, orthologs, or paralogs, whether naturally occurring or non-naturally occurring (e.g., engineered or recombinant), and may include a Cas9 equivalent from any type of CRISPR system (e.g., type II, V, VI), including Cpf1 (a type-V CRISPR-Cas systems), C2c1 (a type V CRISPR-Cas system), C2c2 (a type VI CRISPR-Cas system) and C2c3 (a type V CRISPR-Cas system). Further Cas-equivalents are described in Makarova et al., “C2c2 is a single-component programmable RNA-guided RNA-targeting CRISPR effector,” Science 2016; 353(6299), the contents of which are incorporated herein by reference.

The terms “Cas9” or “Cas9 nuclease” or “Cas9 moiety” or “Cas9 domain” embrace any naturally occurring Cas9 from any organism, any naturally-occurring Cas9 equivalent or functional fragment thereof, any Cas9 homolog, ortholog, or paralog from any organism, and any mutant or variant of a Cas9, naturally-occurring or engineered. The term Cas9 is not meant to be particularly limiting and may be referred to as a “Cas9 or equivalent.” Exemplary Cas9 proteins are further described herein and/or are described in the art and are incorporated herein by reference. The present disclosure is unlimited with regard to the particular Cas9 that is employed in the base editor (PE) of the invention.

As noted herein, Cas9 nuclease sequences and structures are well known to those of skill in the art (see, e.g., “Complete genome sequence of an M1 strain of Streptococcus pyogenes.” Ferretti et al., J. J., McShan W. M., Ajdic D. J., Savic D. J., Savic G., Lyon K., Primeaux C., Sezate S., Suvorov A. N., Kenton S., Lai H. S., Lin S. P., Qian Y., Jia H. G., Najar F. Z., Ren Q., Zhu H., Song L., White J., Yuan X., Clifton S. W., Roe B. A., McLaughlin R. E., Proc. Natl. Acad. Sci. U.S.A. 98:4658-4663(2001); “CRISPR RNA maturation by trans-encoded small RNA and host factor RNase III.” Deltcheva E., Chylinski K., Sharma C. M., Gonzales K., Chao Y., Pirzada Z. A., Eckert M. R., Vogel J., Charpentier E., Nature 471:602-607(2011); and “A programmable dual-RNA-guided DNA endonuclease in adaptive bacterial immunity.” Jinek M., Chylinski K., Fonfara I., Hauer M., Doudna J. A., Charpentier E. Science 337:816-821(2012), the entire contents of each of which are incorporated herein by reference).

Examples of Cas9 and Cas9 equivalents are provided as follows; however, these specific examples are not meant to be limiting. The base editor fusions of the present disclosure may use any suitable napDNAbp, including any suitable Cas9 or Cas9 equivalent.

(1) Wild Type SpCas9

In one embodiment, the base editor constructs described herein may comprise the “canonical SpCas9” nuclease from S. pyogenes, which has been widely used as a tool for genome engineering. This Cas9 protein is a large, multi-domain protein containing two distinct nuclease domains. Point mutations can be introduced into Cas9 to abolish one or both nuclease activities, resulting in a nickase Cas9 (nCas9) or dead Cas9 (dCas9), respectively, that still retains its ability to bind DNA in a sgRNA-programmed manner. In principle, when fused to another protein or domain, Cas9 or variant thereof (e.g., nCas9) can target that protein to virtually any DNA sequence simply by co-expression with an appropriate sgRNA. As used herein, the canonical SpCas9 protein refers to the wild type protein from Streptococcus pyogenes having the following amino acid sequence:

| Description | Sequence | SEQ ID NO: |
|---|---|---|
| SpCas9 | MDKKYSIGLDIGTNSVGWAVITDEYKVPSKKFKVLGNTDRHSIKKNLIGALLFDS | 5 |
| Streptococcus | GETAEATRLKRTARRRYTRRKNRICYLQEIFSNEMAKVDDSFFHRLEESFLVEED | |
| pyogenes | KKHERHPIFGNIVDEVAYHEKYPTIYHLRKKLVDSTDKADLRLIYLALAHMIKFR | |
| M1 | GHFLIEGDLNPDNSDVDKLFIQLVQTYNQLFEENPINASGVDAKAILSARLSKSR | |
| SwissProt | RLENLIAQLPGEKKNGLFGNLIALSLGLTPNFKSNFDLAEDAKLQLSKDTYDDDL | |
| Accession | DNLLAQIGDQYADLFLAAKNLSDAILLSDILRVNTEITKAPLSASMIKRYDEHHQ | |
| No. Q99ZW2 | DLTLLKALVRQQLPEKYKEIFFDQSKNGYAGYIDGGASQEEFYKFIKPILEKMDG | |
| Wild type | TEELLVKLNREDLLRKQRTFDNGSIPHQIHLGELHAILRRQEDFYPFLKDNREKI | |
| | EKILTFRIPYYVGPLARGNSRFAWMTRKSEETITPWNFEEVVDKGASAQSFIERM | |
| | TNFDKNLPNEKVLPKHSLLYEYFTVYNELTKVKYVTEGMRKPAFLSGEQKKAIVD | |
| | LLFKTNRKVTVKQLKEDYFKKIECFDSVEISGVEDRFNASLGTYHDLLKIIKDKD | |
| | FLDNEENEDILEDIVLTLTLFEDREMIEERLKTYAHLFDDKVMKQLKRRRYTGWG | |
| | RLSRKLINGIRDKQSGKTILDFLKSDGFANRNFMQLIHDDSLTFKEDIQKAQVSG | |
| | QGDSLHEHIANLAGSPAIKKGILQTVKVVDELVKVMGRHKPENIVIEMARENQTT | |
| | QKGQKNSRERMKRIEEGIKELGSQILKEHPVENTQLQNEKLYLYYLQNGRDMYVD | |
| | QELDINRLSDYDVDHIVPQSFLKDDSIDNKVLTRSDKNRGKSDNVPSEEVVKKMK | |
| | NYWRQLLNAKLITQRKFDNLTKAERGGLSELDKAGFIKRQLVETRQITKHVAQIL | |
| | DSRMNTKYDENDKLIREVKVITLKSKLVSDFRKDFQFYKVREINNYHHAHDAYLN | |
| | AVVGTALIKKYPKLESEFVYGDYKVYDVRKMIAKSEQEIGKATAKYFFYSNIMNF | |
| | FKTEITLANGEIRKRPLIETNGETGEIVWDKGRDFATVRKVLSMPQVNIVKKTEV | |
| | QTGGFSKESILPKRNSDKLIARKKDWDPKKYGGFDSPTVAYSVLVVAKVEKGKSK | |
| | KLKSVKELLGITIMERSSFEKNPIDFLEAKGYKEVKKDLIIKLPKYSLFELENGR | |
| | KRMLASAGELQKGNELALPSKYVNFLYLASHYEKLKGSPEDNEQKQLFVEQHKHY | |
| | LDEIIEQISEFSKRVILADANLDKVLSAYNKHRDKPIREQAENIIHLFTLTNLGA | |
| | PAAFKYFDTTIDRKRYTSTKEVLDATLIHQSITGLYETRIDLSQLGGD | |
| SpCas9 | ATGGATAAAAAATATAGCATTGGCCTGGATATTGGCACCAACAGCGTGGGCTGGG | 6 |
| Reverse | CGGTGATTACCGATGAATATAAAGTGCCGAGCAAAAAATTTAAAGTGCTGGGCAA | |
| translation | CACCGATCGCCATAGCATTAAAAAAAACCTGATTGGCGCGCTGCTGTTTGATAGC | |
| of | GGCGAAACCGCGGAAGCGACCCGCCTGAAACGCACCGCGCGCCGCCGCTATACCC | |
| SwissProt | GCCGCAAAAACCGCATTTGCTATCTGCAGGAAATTTTTAGCAACGAAATGGCGAA | |
| Accession | AGTGGATGATAGCTTTTTTCATCGCCTGGAAGAAAGCTTTCTGGTGGAAGAAGAT | |
| No. Q99ZW2 | AAAAAACATGAACGCCATCCGATTTTTGGCAACATTGTGGATGAAGTGGCGTATC | |
| Streptococcus | ATGAAAAATATCCGACCATTTATCATCTGCGCAAAAAACTGGTGGATAGCACCGA | |
| pyogenes | TAAAGCGGATCTGCGCCTGATTTATCTGGCGCTGGCGCATATGATTAAATTTCGC | |
| | GGCCATTTTCTGATTGAAGGCGATCTGAACCCGGATAACAGCGATGTGGATAAAC | |
| | TGTTTATTCAGCTGGTGCAGACCTATAACCAGCTGTTTGAAGAAAACCCGATTAA | |
| | CGCGAGCGGCGTGGATGCGAAAGCGATTCTGAGCGCGCGCCTGAGCAAAAGCCGC | |
| | CGCCTGGAAAACCTGATTGCGCAGCTGCCGGGCGAAAAAAAAAACGGCCTGTTTG | |
| | GCAACCTGATTGCGCTGAGCCTGGGCCTGACCCCGAACTTTAAAAGCAACTTTGA | |
| | TCTGGCGGAAGATGCGAAACTGCAGCTGAGCAAAGATACCTATGATGATGATCTG | |
| | GATAACCTGCTGGCGCAGATTGGCGATCAGTATGCGGATCTGTTTCTGGCGGCGA | |
| | AAAACCTGAGCGATGCGATTCTGCTGAGCGATATTCTGCGCGTGAACACCGAAAT | |
| | TACCAAAGCGCCGCTGAGCGCGAGCATGATTAAACGCTATGATGAACATCATCAG | |
| | GATCTGACCCTGCTGAAAGCGCTGGTGCGCCAGCAGCTGCCGGAAAAATATAAAG | |
| | AAATTTTTTTTGATCAGAGCAAAAACGGCTATGCGGGCTATATTGATGGCGGCGC | |
| | GAGCCAGGAAGAATTTTATAAATTTATTAAACCGATTCTGGAAAAAATGGATGGC | |
| | ACCGAAGAACTGCTGGTGAAACTGAACCGCGAAGATCTGCTGCGCAAACAGCGCA | |
| | CCTTTGATAACGGCAGCATTCCGCATCAGATTCATCTGGGCGAACTGCATGCGAT | |
| | TCTGCGCCGCCAGGAAGATTTTTATCCGTTTCTGAAAGATAACCGCGAAAAAATT | |
| | GAAAAAATTCTGACCTTTCGCATTCCGTATTATGTGGGCCCGCTGGCGCGCGGCA | |
| | ACAGCCGCTTTGCGTGGATGACCCGCAAAAGCGAAGAAACCATTACCCCGTGGAA | |
| | CTTTGAAGAAGTGGTGGATAAAGGCGCGAGCGCGCAGAGCTTTATTGAACGCATG | |
| | ACCAACTTTGATAAAAACCTGCCGAACGAAAAAGTGCTGCCGAAACATAGCCTGC | |
| | TGTATGAATATTTTACCGTGTATAACGAACTGACCAAAGTGAAATATGTGACCGA | |
| | AGGCATGCGCAAACCGGCGTTTCTGAGCGGCGAACAGAAAAAAGCGATTGTGGAT | |
| | CTGCTGTTTAAAACCAACCGCAAAGTGACCGTGAAACAGCTGAAAGAAGATTATT | |
| | TTAAAAAAATTGAATGCTTTGATAGCGTGGAAATTAGCGGCGTGGAAGATCGCTT | |
| | TAACGCGAGCCTGGGCACCTATCATGATCTGCTGAAAATTATTAAAGATAAAGAT | |
| | TTTCTGGATAACGAAGAAAACGAAGATATTCTGGAAGATATTGTGCTGACCCTGA | |
| | CCCTGTTTGAAGATCGCGAAATGATTGAAGAACGCCTGAAAACCTATGCGCATCT | |
| | GTTTGATGATAAAGTGATGAAACAGCTGAAACGCCGCCGCTATACCGGCTGGGGC | |
| | CGCCTGAGCCGCAAACTGATTAACGGCATTCGCGATAAACAGAGCGGCAAAACCA | |
| | TTCTGGATTTTCTGAAAAGCGATGGCTTTGCGAACCGCAACTTTATGCAGCTGAT | |
| | TCATGATGATAGCCTGACCTTTAAAGAAGATATTCAGAAAGCGCAGGTGAGCGGC | |
| | CAGGGCGATAGCCTGCATGAACATATTGCGAACCTGGCGGGCAGCCCGGCGATTA | |
| | AAAAAGGCATTCTGCAGACCGTGAAAGTGGTGGATGAACTGGTGAAAGTGATGGG | |
| | CCGCCATAAACCGGAAAACATTGTGATTGAAATGGCGCGCGAAAACCAGACCACC | |
| | CAGAAAGGCCAGAAAAACAGCCGCGAACGCATGAAACGCATTGAAGAAGGCATTA | |
| | AAGAACTGGGCAGCCAGATTCTGAAAGAACATCCGGTGGAAAACACCCAGCTGCA | |
| | GAACGAAAAACTGTATCTGTATTATCTGCAGAACGGCCGCGATATGTATGTGGAT | |
| | CAGGAACTGGATATTAACCGCCTGAGCGATTATGATGTGGATCATATTGTGCCGC | |
| | AGAGCTTTCTGAAAGATGATAGCATTGATAACAAAGTGCTGACCCGCAGCGATAA | |
| | AAACCGCGGCAAAAGCGATAACGTGCCGAGCGAAGAAGTGGTGAAAAAAATGAAA | |
| | AACTATTGGCGCCAGCTGCTGAACGCGAAACTGATTACCCAGCGCAAATTTGATA | |
| | ACCTGACCAAAGCGGAACGCGGCGGCCTGAGCGAACTGGATAAAGCGGGCTTTAT | |
| | TAAACGCCAGCTGGTGGAAACCCGCCAGATTACCAAACATGTGGCGCAGATTCTG | |
| | GATAGCCGCATGAACACCAAATATGATGAAAACGATAAACTGATTCGCGAAGTGA | |
| | AAGTGATTACCCTGAAAAGCAAACTGGTGAGCGATTTTCGCAAAGATTTTCAGTT | |
| | TTATAAAGTGCGCGAAATTAACAACTATCATCATGCGCATGATGCGTATCTGAAC | |
| | GCGGTGGTGGGCACCGCGCTGATTAAAAAATATCCGAAACTGGAAAGCGAATTTG | |
| | TGTATGGCGATTATAAAGTGTATGATGTGCGCAAAATGATTGCGAAAAGCGAACA | |
| | GGAAATTGGCAAAGCGACCGCGAAATATTTTTTTTATAGCAACATTATGAACTTT | |
| | TTTAAAACCGAAATTACCCTGGCGAACGGCGAAATTCGCAAACGCCCGCTGATTG | |
| | AAACCAACGGCGAAACCGGCGAAATTGTGTGGGATAAAGGCCGCGATTTTGCGAC | |
| | CGTGCGCAAAGTGCTGAGCATGCCGCAGGTGAACATTGTGAAAAAAACCGAAGTG | |
| | CAGACCGGCGGCTTTAGCAAAGAAAGCATTCTGCCGAAACGCAACAGCGATAAAC | |
| | TGATTGCGCGCAAAAAAGATTGGGATCCGAAAAAATATGGCGGCTTTGATAGCCC | |
| | GACCGTGGCGTATAGCGTGCTGGTGGTGGCGAAAGTGGAAAAAGGCAAAAGCAAA | |
| | AAACTGAAAAGCGTGAAAGAACTGCTGGGCATTACCATTATGGAACGCAGCAGCT | |
| | TTGAAAAAAACCCGATTGATTTTCTGGAAGCGAAAGGCTATAAAGAAGTGAAAAA | |
| | AGATCTGATTATTAAACTGCCGAAATATAGCCTGTTTGAACTGGAAAACGGCCGC | |
| | AAACGCATGCTGGCGAGCGCGGGCGAACTGCAGAAAGGCAACGAACTGGCGCTGC | |
| | CGAGCAAATATGTGAACTTTCTGTATCTGGCGAGCCATTATGAAAAACTGAAAGG | |
| | CAGCCCGGAAGATAACGAACAGAAACAGCTGTTTGTGGAACAGCATAAACATTAT | |
| | CTGGATGAAATTATTGAACAGATTAGCGAATTTAGCAAACGCGTGATTCTGGCGG | |
| | ATGCGAACCTGGATAAAGTGCTGAGCGCGTATAACAAACATCGCGATAAACCGAT | |
| | TCGCGAACAGGCGGAAAACATTATTCATCTGTTTACCCTGACCAACCTGGGCGCG | |
| | CCGGCGGCGTTTAAATATTTTGATACCACCATTGATCGCAAACGCTATACCAGCA | |
| | CCAAAGAAGTGCTGGATGCGACCCTGATTCATCAGAGCATTACCGGCCTGTATGA | |
| | AACCCGCATTGATCTGAGCCAGCTGGGCGGCGAT | |

The base editors described herein may include canonical SpCas9, or any variant thereof having at least 80%, at least 85%, at least 90%, at least 95%, or at least 99% sequence identity with a wild type Cas9 sequence provided above. These variants may include SpCas9 variants containing one or more mutations, including any known mutation reported with the SwissProt Accession No. Q99ZW2 entry, which include:

| SpCas9 mutation (relative to | Function/Characteristic (as reported) |
|---|---|
| the amino acid sequence | (see UniProtKB - Q99ZW2 |
| of the canonical SpCas9 | (CAS9_STRPT1) entry - |
| sequence, SEQ ID NO: 5) | incorporated herein by reference) |
| D10A | Nickase mutant which cleaves the |
| | protospacer strand (but no cleavage of |
| | non-protospacer strand) |
| S15A | Decreased DNA cleavage activity |
| R66A | Decreased DNA cleavage activity |
| R70A | No DNA cleavage |
| R74A | Decreased DNA cleavage |
| R78A | Decreased DNA cleavage |
| 97-150 deletion | No nuclease activity |
| R165A | Decreased DNA cleavage |
| 175-307 deletion | About 50% decreased DNA cleavage |
| 312-409 deletion | No nuclease activity |
| E762A | Nickase |
| H840A | Nickase mutant which cleaves the non- |
| | protospacer strand but does not cleave |
| | the protospacer strand |
| N854A | Nickase |
| N863A | Nickase |
| H982A | Decreased DNA cleavage |
| D986A | Nickase |
| 1099-1368 deletion | No nuclease activity |
| R1333A | Reduced DNA binding |

Other wild type SpCas9 sequences that may be used in the present disclosure, include:

| Description | Sequence | SEQ ID NO: |
|---|---|---|
| SpCas9 | ATGGATAAGAAATACTCAATAGGCTTAGATATCGGCACAAATAGCGTCGGATGGGCG | 7 |
| Streptococcus | GTGATCACTGATGATTATAAGGTTCCGTCTAAAAAGTTCAAGGTTCTGGGAAATACA | |
| pyogenes | GACCGCCACAGTATCAAAAAAAATCTTATAGGGGCTCTTTTATTTGGCAGTGGAGAG | |
| MGAS1882 | ACAGCGGAAGCGACTCGTCTCAAACGGACAGCTCGTAGAAGGTATACACGTCGGAAG | |
| wild type | AATCGTATTTGTTATCTACAGGAGATTTTTTCAAATGAGATGGCGAAAGTAGATGAT | |
| NC_017053.1 | AGTTTCTTTCATCGACTTGAAGAGTCTTTTTTGGTGGAAGAAGACAAGAAGCATGAA | |
| | CGTCATCCTATTTTTGGAAATATAGTAGATGAAGTTGCTTATCATGAGAAATATCCA | |
| | ACTATCTATCATCTGCGAAAAAAATTGGCAGATTCTACTGATAAAGCGGATTTGCGC | |
| | TTAATCTATTTGGCCTTAGCGCATATGATTAAGTTTCGTGGTCATTTTTTGATTGAG | |
| | GGAGATTTAAATCCTGATAATAGTGATGTGGACAAACTATTTATCCAGTTGGTACAA | |
| | ATCTACAATCAATTATTTGAAGAAAACCCTATTAACGCAAGTAGAGTAGATGCTAAA | |
| | GCGATTCTTTCTGCACGATTGAGTAAATCAAGACGATTAGAAAATCTCATTGCTCAG | |
| | CTCCCCGGTGAGAAGAGAAATGGCTTGTTTGGGAATCTCATTGCTTTGTCATTGGGA | |
| | TTGACCCCTAATTTTAAATCAAATTTTGATTTGGCAGAAGATGCTAAATTACAGCTT | |
| | TCAAAAGATACTTACGATGATGATTTAGATAATTTATTGGCGCAAATTGGAGATCAA | |
| | TATGCTGATTTGTTTTTGGCAGCTAAGAATTTATCAGATGCTATTTTACTTTCAGAT | |
| | ATCCTAAGAGTAAATAGTGAAATAACTAAGGCTCCCCTATCAGCTTCAATGATTAAG | |
| | CGCTACGATGAACATCATCAAGACTTGACTCTTTTAAAAGCTTTAGTTCGACAACAA | |
| | CTTCCAGAAAAGTATAAAGAAATCTTTTTTGATCAATCAAAAAACGGATATGCAGGT | |
| | TATATTGATGGGGGAGCTAGCCAAGAAGAATTTTATAAATTTATCAAACCAATTTTA | |
| | GAAAAAATGGATGGTACTGAGGAATTATTGGTGAAACTAAATCGTGAAGATTTGCTG | |
| | CGCAAGCAACGGACCTTTGACAACGGCTCTATTCCCCATCAAATTCACTTGGGTGAG | |
| | CTGCATGCTATTTTGAGAAGACAAGAAGACTTTTATCCATTTTTAAAAGACAATCGT | |
| | GAGAAGATTGAAAAAATCTTGACTTTTCGAATTCCTTATTATGTTGGTCCATTGGCG | |
| | CGTGGCAATAGTCGTTTTGCATGGATGACTCGGAAGTCTGAAGAAACAATTACCCCA | |
| | TGGAATTTTGAAGAAGTTGTCGATAAAGGTGCTTCAGCTCAATCATTTATTGAACGC | |
| | ATGACAAACTTTGATAAAAATCTTCCAAATGAAAAAGTACTACCAAAACATAGTTTG | |
| | CTTTATGAGTATTTTACGGTTTATAACGAATTGACAAAGGTCAAATATGTTACTGAG | |
| | GGAATGCGAAAACCAGCATTTCTTTCAGGTGAACAGAAGAAAGCCATTGTTGATTTA | |
| | CTCTTCAAAACAAATCGAAAAGTAACCGTTAAGCAATTAAAAGAAGATTATTTCAAA | |
| | AAAATAGAATGTTTTGATAGTGTTGAAATTTCAGGAGTTGAAGATAGATTTAATGCT | |
| | TCATTAGGCGCCTACCATGATTTGCTAAAAATTATTAAAGATAAAGATTTTTTGGAT | |
| | AATGAAGAAAATGAAGATATCTTAGAGGATATTGTTTTAACATTGACCTTATTTGAA | |
| | GATAGGGGGATGATTGAGGAAAGACTTAAAACATATGCTCACCTCTTTGATGATAAG | |
| | GTGATGAAACAGCTTAAACGTCGCCGTTATACTGGTTGGGGACGTTTGTCTCGAAAA | |
| | TTGATTAATGGTATTAGGGATAAGCAATCTGGCAAAACAATATTAGATTTTTTGAAA | |
| | TCAGATGGTTTTGCCAATCGCAATTTTATGCAGCTGATCCATGATGATAGTTTGACA | |
| | TTTAAAGAAGATATTCAAAAAGCACAGGTGTCTGGACAAGGCCATAGTTTACATGAA | |
| | CAGATTGCTAACTTAGCTGGCAGTCCTGCTATTAAAAAAGGTATTTTACAGACTGTA | |
| | AAAATTGTTGATGAACTGGTCAAAGTAATGGGGCATAAGCCAGAAAATATCGTTATT | |
| | GAAATGGCACGTGAAAATCAGACAACTCAAAAGGGCCAGAAAAATTCGCGAGAGCGT | |
| | ATGAAACGAATCGAAGAAGGTATCAAAGAATTAGGAAGTCAGATTCTTAAAGAGCAT | |
| | CCTGTTGAAAATACTCAATTGCAAAATGAAAAGCTCTATCTCTATTATCTACAAAAT | |
| | GGAAGAGACATGTATGTGGACCAAGAATTAGATATTAATCGTTTAAGTGATTATGAT | |
| | GTCGATCACATTGTTCCACAAAGTTTCATTAAAGACGATTCAATAGACAATAAGGTA | |
| | CTAACGCGTTCTGATAAAAATCGTGGTAAATCGGATAACGTTCCAAGTGAAGAAGTA | |
| | GTCAAAAAGATGAAAAACTATTGGAGACAACTTCTAAACGCCAAGTTAATCACTCAA | |
| | CGTAAGTTTGATAATTTAACGAAAGCTGAACGTGGAGGTTTGAGTGAACTTGATAAA | |
| | GCTGGTTTTATCAAACGCCAATTGGTTGAAACTCGCCAAATCACTAAGCATGTGGCA | |
| | CAAATTTTGGATAGTCGCATGAATACTAAATACGATGAAAATGATAAACTTATTCGA | |
| | GAGGTTAAAGTGATTACCTTAAAATCTAAATTAGTTTCTGACTTCCGAAAAGATTTC | |
| | CAATTCTATAAAGTACGTGAGATTAACAATTACCATCATGCCCATGATGCGTATCTA | |
| | AATGCCGTCGTTGGAACTGCTTTGATTAAGAAATATCCAAAACTTGAATCGGAGTTT | |
| | GTCTATGGTGATTATAAAGTTTATGATGTTCGTAAAATGATTGCTAAGTCTGAGCAA | |
| | GAAATAGGCAAAGCAACCGCAAAATATTTCTTTTACTCTAATATCATGAACTTCTTC | |
| | AAAACAGAAATTACACTTGCAAATGGAGAGATTCGCAAACGCCCTCTAATCGAAACT | |
| | AATGGGGAAACTGGAGAAATTGTCTGGGATAAAGGGCGAGATTTTGCCACAGTGCGC | |
| | AAAGTATTGTCCATGCCCCAAGTCAATATTGTCAAGAAAACAGAAGTACAGACAGGC | |
| | GGATTCTCCAAGGAGTCAATTTTACCAAAAAGAAATTCGGACAAGCTTATTGCTCGT | |
| | AAAAAAGACTGGGATCCAAAAAAATATGGTGGTTTTGATAGTCCAACGGTAGCTTAT | |
| | TCAGTCCTAGTGGTTGCTAAGGTGGAAAAAGGGAAATCGAAGAAGTTAAAATCCGTT | |
| | AAAGAGTTACTAGGGATCACAATTATGGAAAGAAGTTCCTTTGAAAAAAATCCGATT | |
| | GACTTTTTAGAAGCTAAAGGATATAAGGAAGTTAAAAAAGACTTAATCATTAAACTA | |
| | CCTAAATATAGTCTTTTTGAGTTAGAAAACGGTCGTAAACGGATGCTGGCTAGTGCC | |
| | GGAGAATTACAAAAAGGAAATGAGCTGGCTCTGCCAAGCAAATATGTGAATTTTTTA | |
| | TATTTAGCTAGTCATTATGAAAAGTTGAAGGGTAGTCCAGAAGATAACGAACAAAAA | |
| | CAATTGTTTGTGGAGCAGCATAAGCATTATTTAGATGAGATTATTGAGCAAATCAGT | |
| | GAATTTTCTAAGCGTGTTATTTTAGCAGATGCCAATTTAGATAAAGTTCTTAGTGCA | |
| | TATAACAAACATAGAGACAAACCAATACGTGAACAAGCAGAAAATATTATTCATTTA | |
| | TTTACGTTGACGAATCTTGGAGCTCCCGCTGCTTTTAAATATTTTGATACAACAATT | |
| | GATCGTAAACGATATACGTCTACAAAAGAAGTTTTAGATGCCACTCTTATCCATCAA | |
| | TCCATCACTGGTCTTTATGAAACACGCATTGATTTGAGTCAGCTAGGAGGTGACTGA | |
| SpCas9 | MDKKYSIGLDIGTNSVGWAVITDDYKVPSKKFKVLGNTDRHSIKKNLIGALLFGSGE | 8 |
| Streptococcus | TAEATRLKRTARRRYTRRKNRICYLQEIFSNEMAKVDDSFFHRLEESFLVEEDKKHE | |
| pyogenes | RHPIFGNIVDEVAYHEKYPTIYHLRKKLADSTDKADLRLIYLALAHMIKFRGHFLIE | |
| MGAS1882 | GDLNPDNSDVDKLFIQLVQIYNQLFEENPINASRVDAKAILSARLSKSRRLENLIAQ | |
| wild type | LPGEKRNGLFGNLIALSLGLTPNFKSNFDLAEDAKLQLSKDTYDDDLDNLLAQIGDQ | |
| NC_017053.1 | YADLFLAAKNLSDAILLSDILRVNSEITKAPLSASMIKRYDEHHQDLTLLKALVRQQ | |
| | LPEKYKEIFFDQSKNGYAGYIDGGASQEEFYKFIKPILEKMDGTEELLVKLNREDLL | |
| | RKQRTFDNGSIPHQIHLGELHAILRRQEDFYPFLKDNREKIEKILTFRIPYYVGPLA | |
| | RGNSRFAWMTRKSEETITPWNFEEVVDKGASAQSFIERMTNFDKNLPNEKVLPKHSL | |
| | LYEYFTVYNELTKVKYVTEGMRKPAFLSGEQKKAIVDLLFKTNRKVTVKQLKEDYFK | |
| | KIECFDSVEISGVEDRFNASLGAYHDLLKIIKDKDFLDNEENEDILEDIVLTLTLFE | |
| | DRGMIEERLKTYAHLFDDKVMKQLKRRRYTGWGRLSRKLINGIRDKQSGKTILDFLK | |
| | SDGFANRNFMQLIHDDSLTFKEDIQKAQVSGQGHSLHEQIANLAGSPAIKKGILQTV | |
| | KIVDELVKVMGHKPENIVIEMARENQTTQKGQKNSRERMKRIEEGIKELGSQILKEH | |
| | PVENTQLQNEKLYLYYLQNGRDMYVDQELDINRLSDYDVDHIVPQSFIKDDSIDNKV | |
| | LTRSDKNRGKSDNVPSEEVVKKMKNYWRQLLNAKLITQRKFDNLTKAERGGLSELDK | |
| | AGFIKRQLVETRQITKHVAQILDSRMNTKYDENDKLIREVKVITLKSKLVSDFRKDF | |
| | QFYKVREINNYHHAHDAYLNAVVGTALIKKYPKLESEFVYGDYKVYDVRKMIAKSEQ | |
| | EIGKATAKYFFYSNIMNFFKTEITLANGEIRKRPLIETNGETGEIVWDKGRDFATVR | |
| | KVLSMPQVNIVKKTEVQTGGFSKESILPKRNSDKLIARKKDWDPKKYGGFDSPTVAY | |
| | SVLVVAKVEKGKSKKLKSVKELLGITIMERSSFEKNPIDFLEAKGYKEVKKDLIIKL | |
| | PKYSLFELENGRKRMLASAGELQKGNELALPSKYVNFLYLASHYEKLKGSPEDNEQK | |
| | QLFVEQHKHYLDEIIEQISEFSKRVILADANLDKVLSAYNKHRDKPIREQAENIIHL | |
| | FTLTNLGAPAAFKYFDTTIDRKRYTSTKEVLDATLIHQSITGLYETRIDLSQLGGD | |
| SpCas9 | ATGGATAAAAAGTATTCTATTGGTTTAGACATCGGCACTAATTCCGTTGGATGGGCT | 9 |
| Streptococcus | GTCATAACCGATGAATACAAAGTACCTTCAAAGAAATTTAAGGTGTTGGGGAACACA | |
| pyogenes | GACCGTCATTCGATTAAAAAGAATCTTATCGGTGCCCTCCTATTCGATAGTGGCGAA | |
| wild type | ACGGCAGAGGCGACTCGCCTGAAACGAACCGCTCGGAGAAGGTATACACGTCGCAAG | |
| SWBC2D7W014 | AACCGAATATGTTACTTACAAGAAATTTTTAGCAATGAGATGGCCAAAGTTGACGAT | |
| | TCTTTCTTTCACCGTTTGGAAGAGTCCTTCCTTGTCGAAGAGGACAAGAAACATGAA | |
| | CGGCACCCCATCTTTGGAAACATAGTAGATGAGGTGGCATATCATGAAAAGTACCCA | |
| | ACGATTTATCACCTCAGAAAAAAGCTAGTTGACTCAACTGATAAAGCGGACCTGAGG | |
| | TTAATCTACTTGGCTCTTGCCCATATGATAAAGTTCCGTGGGCACTTTCTCATTGAG | |
| | GGTGATCTAAATCCGGACAACTCGGATGTCGACAAACTGTTCATCCAGTTAGTACAA | |
| | ACCTATAATCAGTTGTTTGAAGAGAACCCTATAAATGCAAGTGGCGTGGATGCGAAG | |
| | GCTATTCTTAGCGCCCGCCTCTCTAAATCCCGACGGCTAGAAAACCTGATCGCACAA | |
| | TTACCCGGAGAGAAGAAAAATGGGTTGTTCGGTAACCTTATAGCGCTCTCACTAGGC | |
| | CTGACACCAAATTTTAAGTCGAACTTCGACTTAGCTGAAGATGCCAAATTGCAGCTT | |
| | AGTAAGGACACGTACGATGACGATCTCGACAATCTACTGGCACAAATTGGAGATCAG | |
| | TATGCGGACTTATTTTTGGCTGCCAAAAACCTTAGCGATGCAATCCTCCTATCTGAC | |
| | ATACTGAGAGTTAATACTGAGATTACCAAGGCGCCGTTATCCGCTTCAATGATCAAA | |
| | AGGTACGATGAACATCACCAAGACTTGACACTTCTCAAGGCCCTAGTCCGTCAGCAA | |
| | CTGCCTGAGAAATATAAGGAAATATTCTTTGATCAGTCGAAAAACGGGTACGCAGGT | |
| | TATATTGACGGCGGAGCGAGTCAAGAGGAATTCTACAAGTTTATCAAACCCATATTA | |
| | GAGAAGATGGATGGGACGGAAGAGTTGCTTGTAAAACTCAATCGCGAAGATCTACTG | |
| | CGAAAGCAGCGGACTTTCGACAACGGTAGCATTCCACATCAAATCCACTTAGGCGAA | |
| | TTGCATGCTATACTTAGAAGGCAGGAGGATTTTTATCCGTTCCTCAAAGACAATCGT | |
| | GAAAAGATTGAGAAAATCCTAACCTTTCGCATACCTTACTATGTGGGACCCCTGGCC | |
| | CGAGGGAACTCTCGGTTCGCATGGATGACAAGAAAGTCCGAAGAAACGATTACTCCA | |
| | TGGAATTTTGAGGAAGTTGTCGATAAAGGTGCGTCAGCTCAATCGTTCATCGAGAGG | |
| | ATGACCAACTTTGACAAGAATTTACCGAACGAAAAAGTATTGCCTAAGCACAGTTTA | |
| | CTTTACGAGTATTTCACAGTGTACAATGAACTCACGAAAGTTAAGTATGTCACTGAG | |
| | GGCATGCGTAAACCCGCCTTTCTAAGCGGAGAACAGAAGAAAGCAATAGTAGATCTG | |
| | TTATTCAAGACCAACCGCAAAGTGACAGTTAAGCAATTGAAAGAGGACTACTTTAAG | |
| | AAAATTGAATGCTTCGATTCTGTCGAGATCTCCGGGGTAGAAGATCGATTTAATGCG | |
| | TCACTTGGTACGTATCATGACCTCCTAAAGATAATTAAAGATAAGGACTTCCTGGAT | |
| | AACGAAGAGAATGAAGATATCTTAGAAGATATAGTGTTGACTCTTACCCTCTTTGAA | |
| | GATCGGGAAATGATTGAGGAAAGACTAAAAACATACGCTCACCTGTTCGACGATAAG | |
| | GTTATGAAACAGTTAAAGAGGCGTCGCTATACGGGCTGGGGACGATTGTCGCGGAAA | |
| | CTTATCAACGGGATAAGAGACAAGCAAAGTGGTAAAACTATTCTCGATTTTCTAAAG | |
| | AGCGACGGCTTCGCCAATAGGAACTTTATGCAGCTGATCCATGATGACTCTTTAACC | |
| | TTCAAAGAGGATATACAAAAGGCACAGGTTTCCGGACAAGGGGACTCATTGCACGAA | |
| | CATATTGCGAATCTTGCTGGTTCGCCAGCCATCAAAAAGGGCATACTCCAGACAGTC | |
| | AAAGTAGTGGATGAGCTAGTTAAGGTCATGGGACGTCACAAACCGGAAAACATTGTA | |
| | ATCGAGATGGCACGCGAAAATCAAACGACTCAGAAGGGGCAAAAAAACAGTCGAGAG | |
| | CGGATGAAGAGAATAGAAGAGGGTATTAAAGAACTGGGCAGCCAGATCTTAAAGGAG | |
| | CATCCTGTGGAAAATACCCAATTGCAGAACGAGAAACTTTACCTCTATTACCTACAA | |
| | AATGGAAGGGACATGTATGTTGATCAGGAACTGGACATAAACCGTTTATCTGATTAC | |
| | GACGTCGATCACATTGTACCCCAATCCTTTTTGAAGGACGATTCAATCGACAATAAA | |
| | GTGCTTACACGCTCGGATAAGAACCGAGGGAAAAGTGACAATGTTCCAAGCGAGGAA | |
| | GTCGTAAAGAAAATGAAGAACTATTGGCGGCAGCTCCTAAATGCGAAACTGATAACG | |
| | CAAAGAAAGTTCGATAACTTAACTAAAGCTGAGAGGGGTGGCTTGTCTGAACTTGAC | |
| | AAGGCCGGATTTATTAAACGTCAGCTCGTGGAAACCCGCCAAATCACAAAGCATGTT | |
| | GCACAGATACTAGATTCCCGAATGAATACGAAATACGACGAGAACGATAAGCTGATT | |
| | CGGGAAGTCAAAGTAATCACTTTAAAGTCAAAATTGGTGTCGGACTTCAGAAAGGAT | |
| | TTTCAATTCTATAAAGTTAGGGAGATAAATAACTACCACCATGCGCACGACGCTTAT | |
| | CTTAATGCCGTCGTAGGGACCGCACTCATTAAGAAATACCCGAAGCTAGAAAGTGAG | |
| | TTTGTGTATGGTGATTACAAAGTTTATGACGTCCGTAAGATGATCGCGAAAAGCGAA | |
| | CAGGAGATAGGCAAGGCTACAGCCAAATACTTCTTTTATTCTAACATTATGAATTTC | |
| | TTTAAGACGGAAATCACTCTGGCAAACGGAGAGATACGCAAACGACCTTTAATTGAA | |
| | ACCAATGGGGAGACAGGTGAAATCGTATGGGATAAGGGCCGGGACTTCGCGACGGTG | |
| | AGAAAAGTTTTGTCCATGCCCCAAGTCAACATAGTAAAGAAAACTGAGGTGCAGACC | |
| | GGAGGGTTTTCAAAGGAATCGATTCTTCCAAAAAGGAATAGTGATAAGCTCATCGCT | |
| | CGTAAAAAGGACTGGGACCCGAAAAAGTACGGTGGCTTCGATAGCCCTACAGTTGCC | |
| | TATTCTGTCCTAGTAGTGGCAAAAGTTGAGAAGGGAAAATCCAAGAAACTGAAGTCA | |
| | GTCAAAGAATTATTGGGGATAACGATTATGGAGCGCTCGTCTTTTGAAAAGAACCCC | |
| | ATCGACTTCCTTGAGGCGAAAGGTTACAAGGAAGTAAAAAAGGATCTCATAATTAAA | |
| | CTACCAAAGTATAGTCTGTTTGAGTTAGAAAATGGCCGAAAACGGATGTTGGCTAGC | |
| | GCCGGAGAGCTTCAAAAGGGGAACGAACTCGCACTACCGTCTAAATACGTGAATTTC | |
| | CTGTATTTAGCGTCCCATTACGAGAAGTTGAAAGGTTCACCTGAAGATAACGAACAG | |
| | AAGCAACTTTTTGTTGAGCAGCACAAACATTATCTCGACGAAATCATAGAGCAAATT | |
| | TCGGAATTCAGTAAGAGAGTCATCCTAGCTGATGCCAATCTGGACAAAGTATTAAGC | |
| | GCATACAACAAGCACAGGGATAAACCCATACGTGAGCAGGCGGAAAATATTATCCAT | |
| | TTGTTTACTCTTACCAACCTCGGCGCTCCAGCCGCATTCAAGTATTTTGACACAACG | |
| | ATAGATCGCAAACGATACACTTCTACCAAGGAGGTGCTAGACGCGACACTGATTCAC | |
| | CAATCCATCACGGGATTATATGAAACTCGGATAGATTTGTCACAGCTTGGGGGTGAC | |
| | GGATCCCCCAAGAAGAAGAGGAAAGTCTCGAGCGACTACAAAGACCATGACGGTGAT | |
| | TATAAAGATCATGACATCGATTACAAGGATGACGATGACAAGGCTGCAGGA | |
| SpCas9 | MDKKYSIGLDIGTNSVGWAVITDEYKVPSKKFKVLGNTDRHSIKKNLIGALLFDSGE | 10 |
| Streptococcus | TAEATRLKRTARRRYTRRKNRICYLQEIFSNEMAKVDDSFFHRLEESFLVEEDKKHE | |
| pyogenes | RHPIFGNIVDEVAYHEKYPTIYHLRKKLVDSTDKADLRLIYLALAHMIKFRGHFLIE | |
| wild type | GDLNPDNSDVDKLFIQLVQTYNQLFEENPINASGVDAKAILSARLSKSRRLENLIAQ | |
| Encoded | LPGEKKNGLFGNLIALSLGLTPNFKSNFDLAEDAKLQLSKDTYDDDLDNLLAQIGDQ | |
| product of | YADLFLAAKNLSDAILLSDILRVNTEITKAPLSASMIKRYDEHHQDLTLLKALVRQQ | |
| SWBC2D7W014 | LPEKYKEIFFDQSKNGYAGYIDGGASQEEFYKFIKPILEKMDGTEELLVKLNREDLL | |
| | RKQRTFDNGSIPHQIHLGELHAILRRQEDFYPFLKDNREKIEKILTFRIPYYVGPLA | |
| | RGNSRFAWMTRKSEETITPWNFEEVVDKGASAQSFIERMTNFDKNLPNEKVLPKHSL | |
| | LYEYFTVYNELTKVKYVTEGMRKPAFLSGEQKKAIVDLLFKTNRKVTVKQLKEDYFK | |
| | KIECFDSVEISGVEDRFNASLGTYHDLLKIIKDKDFLDNEENEDILEDIVLTLTLFE | |
| | DREMIEERLKTYAHLFDDKVMKQLKRRRYTGWGRLSRKLINGIRDKQSGKTILDFLK | |
| | SDGFANRNFMQLIHDDSLTFKEDIQKAQVSGQGDSLHEHIANLAGSPAIKKGILQTV | |
| | KVVDELVKVMGRHKPENIVIEMARENQTTQKGQKNSRERMKRIEEGIKELGSQILKE | |
| | HPVENTQLQNEKLYLYYLQNGRDMYVDQELDINRLSDYDVDHIVPQSFLKDDSIDNK | |
| | VLTRSDKNRGKSDNVPSEEVVKKMKNYWRQLLNAKLITQRKFDNLTKAERGGLSELD | |
| | KAGFIKRQLVETRQITKHVAQILDSRMNTKYDENDKLIREVKVITLKSKLVSDFRKD | |
| | FQFYKVREINNYHHAHDAYLNAVVGTALIKKYPKLESEFVYGDYKVYDVRKMIAKSE | |
| | QEIGKATAKYFFYSNIMNFFKTEITLANGEIRKRPLIETNGETGEIVWDKGRDFATV | |
| | RKVLSMPQVNIVKKTEVQTGGFSKESILPKRNSDKLIARKKDWDPKKYGGFDSPTVA | |
| | YSVLVVAKVEKGKSKKLKSVKELLGITIMERSSFEKNPIDFLEAKGYKEVKKDLIIK | |
| | LPKYSLFELENGRKRMLASAGELQKGNELALPSKYVNFLYLASHYEKLKGSPEDNEQ | |
| | KQLFVEQHKHYLDEIIEQISEFSKRVILADANLDKVLSAYNKHRDKPIREQAENIIH | |
| | LFTLTNLGAPAAFKYFDTTIDRKRYTSTKEVLDATLIHQSITGLYETRIDLSQLGGD | |
| | GSPKKKRKVSSDYKDHDGDYKDHDIDYKDDDDKAAG | |
| SpCas9 | ATGGATAAGAAATACTCAATAGGCTTAGATATCGGCACAAATAGCGTCGGATGGGCG | 11 |
| Streptococcus | GTGATCACTGATGAATATAAGGTTCCGTCTAAAAAGTTCAAGGTTCTGGGAAATACA | |
| pyogenes | GACCGCCACAGTATCAAAAAAAATCTTATAGGGGCTCTTTTATTTGACAGTGGAGAG | |
| M1GAS wild | ACAGCGGAAGCGACTCGTCTCAAACGGACAGCTCGTAGAAGGTATACACGTCGGAAG | |
| type | AATCGTATTTGTTATCTACAGGAGATTTTTTCAAATGAGATGGCGAAAGTAGATGAT | |
| NC002737.2 | AGTTTCTTTCATCGACTTGAAGAGTCTTTTTTGGTGGAAGAAGACAAGAAGCATGAA | |
| | CGTCATCCTATTTTTGGAAATATAGTAGATGAAGTTGCTTATCATGAGAAATATCCA | |
| | ACTATCTATCATCTGCGAAAAAAATTGGTAGATTCTACTGATAAAGCGGATTTGCGC | |
| | TTAATCTATTTGGCCTTAGCGCATATGATTAAGTTTCGTGGTCATTTTTTGATTGAG | |
| | GGAGATTTAAATCCTGATAATAGTGATGTGGACAAACTATTTATCCAGTTGGTACAA | |
| | ACCTACAATCAATTATTTGAAGAAAACCCTATTAACGCAAGTGGAGTAGATGCTAAA | |
| | GCGATTCTTTCTGCACGATTGAGTAAATCAAGACGATTAGAAAATCTCATTGCTCAG | |
| | CTCCCCGGTGAGAAGAAAAATGGCTTATTTGGGAATCTCATTGCTTTGTCATTGGGT | |
| | TTGACCCCTAATTTTAAATCAAATTTTGATTTGGCAGAAGATGCTAAATTACAGCTT | |
| | TCAAAAGATACTTACGATGATGATTTAGATAATTTATTGGCGCAAATTGGAGATCAA | |
| | TATGCTGATTTGTTTTTGGCAGCTAAGAATTTATCAGATGCTATTTTACTTTCAGAT | |
| | ATCCTAAGAGTAAATACTGAAATAACTAAGGCTCCCCTATCAGCTTCAATGATTAAA | |
| | CGCTACGATGAACATCATCAAGACTTGACTCTTTTAAAAGCTTTAGTTCGACAACAA | |
| | CTTCCAGAAAAGTATAAAGAAATCTTTTTTGATCAATCAAAAAACGGATATGCAGGT | |
| | TATATTGATGGGGGAGCTAGCCAAGAAGAATTTTATAAATTTATCAAACCAATTTTA | |
| | GAAAAAATGGATGGTACTGAGGAATTATTGGTGAAACTAAATCGTGAAGATTTGCTG | |
| | CGCAAGCAACGGACCTTTGACAACGGCTCTATTCCCCATCAAATTCACTTGGGTGAG | |
| | CTGCATGCTATTTTGAGAAGACAAGAAGACTTTTATCCATTTTTAAAAGACAATCGT | |
| | GAGAAGATTGAAAAAATCTTGACTTTTCGAATTCCTTATTATGTTGGTCCATTGGCG | |
| | CGTGGCAATAGTCGTTTTGCATGGATGACTCGGAAGTCTGAAGAAACAATTACCCCA | |
| | TGGAATTTTGAAGAAGTTGTCGATAAAGGTGCTTCAGCTCAATCATTTATTGAACGC | |
| | ATGACAAACTTTGATAAAAATCTTCCAAATGAAAAAGTACTACCAAAACATAGTTTG | |
| | CTTTATGAGTATTTTACGGTTTATAACGAATTGACAAAGGTCAAATATGTTACTGAA | |
| | GGAATGCGAAAACCAGCATTTCTTTCAGGTGAACAGAAGAAAGCCATTGTTGATTTA | |
| | CTCTTCAAAACAAATCGAAAAGTAACCGTTAAGCAATTAAAAGAAGATTATTTCAAA | |
| | AAAATAGAATGTTTTGATAGTGTTGAAATTTCAGGAGTTGAAGATAGATTTAATGCT | |
| | TCATTAGGTACCTACCATGATTTGCTAAAAATTATTAAAGATAAAGATTTTTTGGAT | |
| | AATGAAGAAAATGAAGATATCTTAGAGGATATTGTTTTAACATTGACCTTATTTGAA | |
| | GATAGGGAGATGATTGAGGAAAGACTTAAAACATATGCTCACCTCTTTGATGATAAG | |
| | GTGATGAAACAGCTTAAACGTCGCCGTTATACTGGTTGGGGACGTTTGTCTCGAAAA | |
| | TTGATTAATGGTATTAGGGATAAGCAATCTGGCAAAACAATATTAGATTTTTTGAAA | |
| | TCAGATGGTTTTGCCAATCGCAATTTTATGCAGCTGATCCATGATGATAGTTTGACA | |
| | TTTAAAGAAGACATTCAAAAAGCACAAGTGTCTGGACAAGGCGATAGTTTACATGAA | |
| | CATATTGCAAATTTAGCTGGTAGCCCTGCTATTAAAAAAGGTATTTTACAGACTGTA | |
| | AAAGTTGTTGATGAATTGGTCAAAGTAATGGGGCGGCATAAGCCAGAAAATATCGTT | |
| | ATTGAAATGGCACGTGAAAATCAGACAACTCAAAAGGGCCAGAAAAATTCGCGAGAG | |
| | CGTATGAAACGAATCGAAGAAGGTATCAAAGAATTAGGAAGTCAGATTCTTAAAGAG | |
| | CATCCTGTTGAAAATACTCAATTGCAAAATGAAAAGCTCTATCTCTATTATCTCCAA | |
| | AATGGAAGAGACATGTATGTGGACCAAGAATTAGATATTAATCGTTTAAGTGATTAT | |
| | GATGTCGATCACATTGTTCCACAAAGTTTCCTTAAAGACGATTCAATAGACAATAAG | |
| | GTCTTAACGCGTTCTGATAAAAATCGTGGTAAATCGGATAACGTTCCAAGTGAAGAA | |
| | GTAGTCAAAAAGATGAAAAACTATTGGAGACAACTTCTAAACGCCAAGTTAATCACT | |
| | CAACGTAAGTTTGATAATTTAACGAAAGCTGAACGTGGAGGTTTGAGTGAACTTGAT | |
| | AAAGCTGGTTTTATCAAACGCCAATTGGTTGAAACTCGCCAAATCACTAAGCATGTG | |
| | GCACAAATTTTGGATAGTCGCATGAATACTAAATACGATGAAAATGATAAACTTATT | |
| | CGAGAGGTTAAAGTGATTACCTTAAAATCTAAATTAGTTTCTGACTTCCGAAAAGAT | |
| | TTCCAATTCTATAAAGTACGTGAGATTAACAATTACCATCATGCCCATGATGCGTAT | |
| | CTAAATGCCGTCGTTGGAACTGCTTTGATTAAGAAATATCCAAAACTTGAATCGGAG | |
| | TTTGTCTATGGTGATTATAAAGTTTATGATGTTCGTAAAATGATTGCTAAGTCTGAG | |
| | CAAGAAATAGGCAAAGCAACCGCAAAATATTTCTTTTACTCTAATATCATGAACTTC | |
| | TTCAAAACAGAAATTACACTTGCAAATGGAGAGATTCGCAAACGCCCTCTAATCGAA | |
| | ACTAATGGGGAAACTGGAGAAATTGTCTGGGATAAAGGGCGAGATTTTGCCACAGTG | |
| | CGCAAAGTATTGTCCATGCCCCAAGTCAATATTGTCAAGAAAACAGAAGTACAGACA | |
| | GGCGGATTCTCCAAGGAGTCAATTTTACCAAAAAGAAATTCGGACAAGCTTATTGCT | |
| | CGTAAAAAAGACTGGGATCCAAAAAAATATGGTGGTTTTGATAGTCCAACGGTAGCT | |
| | TATTCAGTCCTAGTGGTTGCTAAGGTGGAAAAAGGGAAATCGAAGAAGTTAAAATCC | |
| | GTTAAAGAGTTACTAGGGATCACAATTATGGAAAGAAGTTCCTTTGAAAAAAATCCG | |
| | ATTGACTTTTTAGAAGCTAAAGGATATAAGGAAGTTAAAAAAGACTTAATCATTAAA | |
| | CTACCTAAATATAGTCTTTTTGAGTTAGAAAACGGTCGTAAACGGATGCTGGCTAGT | |
| | GCCGGAGAATTACAAAAAGGAAATGAGCTGGCTCTGCCAAGCAAATATGTGAATTTT | |
| | TTATATTTAGCTAGTCATTATGAAAAGTTGAAGGGTAGTCCAGAAGATAACGAACAA | |
| | AAACAATTGTTTGTGGAGCAGCATAAGCATTATTTAGATGAGATTATTGAGCAAATC | |
| | AGTGAATTTTCTAAGCGTGTTATTTTAGCAGATGCCAATTTAGATAAAGTTCTTAGT | |
| | GCATATAACAAACATAGAGACAAACCAATACGTGAACAAGCAGAAAATATTATTCAT | |
| | TTATTTACGTTGACGAATCTTGGAGCTCCCGCTGCTTTTAAATATTTTGATACAACA | |
| | ATTGATCGTAAACGATATACGTCTACAAAAGAAGTTTTAGATGCCACTCTTATCCAT | |
| | CAATCCATCACTGGTCTTTATGAAACACGCATTGATTTGAGTCAGCTAGGAGGTGAC | |
| | TGA | |
| SpCas9 | MDKKYSIGLDIGTNSVGWAVITDEYKVPSKKFKVLGNTDRHSIKKNLIGALLFDSGE | 12 |
| Streptococcus | TAEATRLKRTARRRYTRRKNRICYLQEIFSNEMAKVDDSFFHRLEESFLVEEDKKHE | |
| pyogenes | RHPIFGNIVDEVAYHEKYPTIYHLRKKLVDSTDKADLRLIYLALAHMIKFRGHFLIE | |
| M1GAS wild | GDLNPDNSDVDKLFIQLVQTYNQLFEENPINASGVDAKAILSARLSKSRRLENLIAQ | |
| type | LPGEKKNGLFGNLIALSLGLTPNFKSNFDLAEDAKLQLSKDTYDDDLDNLLAQIGDQ | |
| Encoded | YADLFLAAKNLSDAILLSDILRVNTEITKAPLSASMIKRYDEHHQDLTLLKALVRQQ | |
| product of | LPEKYKEIFFDQSKNGYAGYIDGGASQEEFYKFIKPILEKMDGTEELLVKLNREDLL | |
| NC_002737.2 | RKQRTFDNGSIPHQIHLGELHAILRRQEDFYPFLKDNREKIEKILTFRIPYYVGPLA | |
| (100% | RGNSRFAWMTRKSEETITPWNFEEVVDKGASAQSFIERMTNFDKNLPNEKVLPKHSL | |
| identical to | LYEYFTVYNELTKVKYVTEGMRKPAFLSGEQKKAIVDLLFKTNRKVTVKQLKEDYFK | |
| the | KIECFDSVEISGVEDRFNASLGTYHDLLKIIKDKDFLDNEENEDILEDIVLTLTLFE | |
| canonical | DREMIEERLKTYAHLFDDKVMKQLKRRRYTGWGRLSRKLINGIRDKQSGKTILDFLK | |
| Q99ZW2 | SDGFANRNFMQLIHDDSLTFKEDIQKAQVSGQGDSLHEHIANLAGSPAIKKGILQTV | |
| wild type) | KVVDELVKVMGRHKPENIVIEMARENQTTQKGQKNSRERMKRIEEGIKELGSQILKE | |
| | HPVENTQLQNEKLYLYYLQNGRDMYVDQELDINRLSDYDVDHIVPQSFLKDDSIDNK | |
| | VLTRSDKNRGKSDNVPSEEVVKKMKNYWRQLLNAKLITQRKFDNLTKAERGGLSELD | |
| | KAGFIKRQLVETRQITKHVAQILDSRMNTKYDENDKLIREVKVITLKSKLVSDFRKD | |
| | FQFYKVREINNYHHAHDAYLNAVVGTALIKKYPKLESEFVYGDYKVYDVRKMIAKSE | |
| | QEIGKATAKYFFYSNIMNFFKTEITLANGEIRKRPLIETNGETGEIVWDKGRDFATV | |
| | RKVLSMPQVNIVKKTEVQTGGFSKESILPKRNSDKLIARKKDWDPKKYGGFDSPTVA | |
| | YSVLVVAKVEKGKSKKLKSVKELLGITIMERSSFEKNPIDFLEAKGYKEVKKDLIIK | |
| | LPKYSLFELENGRKRMLASAGELQKGNELALPSKYVNFLYLASHYEKLKGSPEDNEQ | |
| | KQLFVEQHKHYLDEIIEQISEFSKRVILADANLDKVLSAYNKHRDKPIREQAENIIH | |
| | LFTLTNLGAPAAFKYFDTTIDRKRYTSTKEVLDATLIHQSITGLYETRIDLSQLGGD | |

The base editors described herein may include any of the above SpCas9 sequences, or any variant thereof having at least 80%, at least 85%, at least 90%, at least 95%, or at least 99% sequence identity thereto.

(2) Wild Type Cas9 Orthologs

In other embodiments, the Cas9 protein can be a wild type Cas9 ortholog from another bacterial species. For example, the following Cas9 orthologs can be used in connection with the base editor constructs described in this specification. In addition, any variant Cas9 orthologs having at least 80%, at least 85%, at least 90%, at least 95% or at least 99% sequence identity to any of the below orthologs may also be used with the present base editors.

| Description | Sequence |
|---|---|
| LfCas9 | MKEYHIGLDIGTSSIGWAVTDSQFKLMRIKGKTAIGVRLFEEGKTAAERRTFRTTRRRLKRRKWRLHYLDEIFAPHLQEVD |
| Lactobacillus | ENFLRRLKQSNIHPEDPTKNQAFIGKLLFPDLLKKNERGYPTLIKMRDELPVEQRAHYPVMNIYKLREAMINEDRQFDLRE |
| fermentum | VYLAVHHIVKYRGHFLNNASVDKFKVGRIDFDKSFNVLNEAYEELQNGEGSFTIEPSKVEKIGQLLLDTKMRKLDRQKAVA |
| wild type | KLLEVKVADKEETKRNKQIATAMSKLVLGYKADFATVAMANGNEWKIDLSSETSEDEIEKFREELSDAQNDILTEITSLFS |
| GenBank: | QIMLNEIVPNGMSISESMMDRYWTHERQLAEVKEYLATQPASARKEFDQVYNKYIGQAPKERGFDLEKGLKKILSKKENWK |
| SNX31424.11 | EIDELLKAGDFLPKQRTSANGVIPHQMHQQELDRIIEKQAKYYPWLATENPATGERDRHQAKYELDQLVSFRIPYYVGPLV |
| | TPEVQKATSGAKFAWAKRKEDGEITPWNLWDKIDRAESAEAFIKRMTVKDTYLLNEDVLPANSLLYQKYNVLNELNNVRVN |
| | GRRLSVGIKQDIYTELFKKKKTVKASDVASLVMAKTRGVNKPSVEGLSDPKKFNSNLATYLDLKSIVGDKVDDNRYQTDLE |
| | NIIEWRSVFEDGEIFADKLTEVEWLTDEQRSALVKKRYKGWGRLSKKLLTGIVDENGQRIIDLMWNTDQNFKEIVDQPVFK |
| | EQIDQLNQKAITNDGMTLRERVESVLDDAYTSPQNKKAIWQVVRVVEDIVKAVGNAPKSISIEFARNEGNKGEITRSRRTQ |
| | LQKLFEDQAHELVKDTSLTEELEKAPDLSDRYYFYFTQGGKDMYTGDPINFDEISTKYDIDHILPQSFVKDNSLDNRVLTS |
| | RKENNKKSDQVPAKLYAAKMKPYWNQLLKQGLITQRKFENLTKDVDQNIKYRSLGFVKRQLVETRQVIKLTANILGSMYQE |
| | AGTEIIETRAGLTKQLREEFDLPKVREVNDYHHAVDAYLTTFAGQYLNRRYPKLRSFFVYGEYMKFKHGSDLKLRNFNFFH |
| | ELMEGDKSQGKVVDQQTGELITTRDEVAKSFDRLLNMKYMLVSKEVHDRSDQLYGATIVTAKESGKLTSPIEIKKNRLVDL |
| | YGAYTNGTSAFMTIIKFTGNKPKYKVIGIPTTSAASLKRAGKPGSESYNQELHRIIKSNPKVKKGFEIVVPHVSYGQLIVD |
| | GDCKFTLASPTVQHPATQLVLSKKSLETISSGYKILKDKPAIANERLIRVFDEVVGQMNRYFTIFDQRSNRQKVADARDKF |
| | LSLPTESKYEGAKKVQVGKTEVITNLLMGLHANATQGDLKVLGLATFGFFQSTTGLSLSEDTMIVYQSPTGLFERRICLKD |
| | I(SEQ ID NO: 13) |
| SaCas9 | MDKKYSIGLDIGTNSVGWAVITDEYKVPSKKFKVLGNTDRHSIKKNLIGALLFDSGETAEATRLKRTARRRYTRRKNRICY |
| Staphylococcus | LQEIFSNEMAKVDDSFFHRLEESFLVEEDKKHERHPIFGNIVDEVAYHEKYPTIYHLRKKLVDSTDKADLRLIYLALAHMI |
| aureus | KFRGHFLIEGDLNPDNSDVDKLFIQLVQTYNQLFEENPINASGVDAKAILSARLSKSRRLENLIAQLPGEKKNGLFGNLIA |
| wild type | LSLGLTPNFKSNFDLAEDAKLQLSKDTYDDDLDNLLAQIGDQYADLFLAAKNLSDAILLSDILRVNTEITKAPLSASMIKR |
| GenBank: | YDEHHQDLTLLKALVRQQLPEKYKEIFFDQSKNGYAGYIDGGASQEEFYKFIKPILEKMDGTEELLVKLNREDLLRKQRTF |
| AYD60528.1 | DNGSIPHQIHLGELHAILRRQEDFYPFLKDNREKIEKILTFRIPYYVGPLARGNSRFAWMTRKSEETITPWNFEEVVDKGA |
| | SAQSFIERMTNFDKNLPNEKVLPKHSLLYEYFTVYNELTKVKYVTEGMRKPAFLSGEQKKAIVDLLFKTNRKVTVKQLKED |
| | YFKKIECFDSVEISGVEDRFNASLGTYHDLLKIIKDKDFLDNEENEDILEDIVLTLTLFEDREMIEERLKTYAHLFDDKVM |
| | KQLKRRRYTGWGRLSRKLINGIRDKQSGKTILDFLKSDGFANRNFMQLIHDDSLTFKEDIQKAQVSGQGDSLHEHIANLAG |
| | SPAIKKGILQTVKVVDELVKVMGRHKPENIVIEMARENQTTQKGQKNSRERMKRIEEGIKELGSQILKEHPVENTQLQNEK |
| | LYLYYLQNGRDMYVDQELDINRLSDYDVDHIVPQSFLKDDSIDNKVLTRSDKNRGKSDNVPSEEVVKKMKNYWRQLLNAKL |
| | ITQRKFDNLTKAERGGLSELDKAGFIKRQLVETRQITKHVAQILDSRMNTKYDENDKLIREVKVITLKSKLVSDFRKDFQF |
| | YKVREINNYHHAHDAYLNAVVGTALIKKYPKLESEFVYGDYKVYDVRKMIAKSEQEIGKATAKYFFYSNIMNFFKTEITLA |
| | NGEIRKRPLIETNGETGEIVWDKGRDFATVRKVLSMPQVNIVKKTEVQTGGFSKESILPKRNSDKLIARKKDWDPKKYGGF |
| | DSPTVAYSVLVVAKVEKGKSKKLKSVKELLGITIMERSSFEKNPIDFLEAKGYKEVKKDLIIKLPKYSLFELENGRKRMLA |
| | SAGELQKGNELALPSKYVNFLYLASHYEKLKGSPEDNEQKQLFVEQHKHYLDEIIEQISEFSKRVILADANLDKVLSAYNK |
| | HRDKPIREQAENIIHLFTLTNLGAPAAFKYFDTTIDRKRYTSTKEVLDATLIHQSITGLYETRIDLSQLGGD(SEQ ID |
| | NO: 14) |
| SaCas9 | MGKRNYILGLDIGITSVGYGIIDYETRDVIDAGVRLFKEANVENNEGRRSKRGARRLKRRRRHRIQRVKKLLFDYNLLTDH |
| Staphylococcus | SELSGINPYEARVKGLSQKLSEEEFSAALLHLAKRRGVHNVNEVEEDTGNELSTKEQISRNSKALEEKYVAELQLERLKKD |
| aureus | GEVRGSINRFKTSDYVKEAKQLLKVQKAYHQLDQSFIDTYIDLLETRRTYYEGPGEGSPFGWKDIKEWYEMLMGHCTYFPE |
| | ELRSVKYAYNADLYNALNDLNNLVITRDENEKLEYYEKFQIIENVFKQKKKPTLKQIAKEILVNEEDIKGYRVTSTGKPEF |
| | TNLKVYHDIKDITARKEIIENAELLDQIAKILTIYQSSEDIQEELTNLNSELTQEEIEQISNLKGYTGTHNLSLKAINLIL |
| | DELWHTNDNQIAIFNRLKLVPKKVDLSQQKEIPTTLVDDFILSPVVKRSFIQSIKVINAIIKKYGLPNDIIIELAREKNSK |
| | DAQKMINEMQKRNRQTNERIEEIIRTTGKENAKYLIEKIKLHDMQEGKCLYSLEAIPLEDLLNNPFNYEVDHIIPRSVSFD |
| | NSFNNKVLVKQEENSKKGNRTPFQYLSSSDSKISYETFKKHILNLAKGKGRISKTKKEYLLEERDINRFSVQKDFINRNLV |
| | DTRYATRGLMNLLRSYFRVNNLDVKVKSINGGFTSFLRRKWKFKKERNKGYKHHAEDALIIANADFIFKEWKKLDKAKKVM |
| | ENQMFEEKQAESMPEIETEQEYKEIFITPHQIKHIKDFKDYKYSHRVDKKPNRKLINDTLYSTRKDDKGNTLIVNNLNGLY |
| | DKDNDKLKKLINKSPEKLLMYHHDPQTYQKLKLIMEQYGDEKNPLYKYYEETGNYLTKYSKKDNGPVIKKIKYYGNKLNAH |
| | LDITDDYPNSRNKVVKLSLKPYRFDVYLDNGVYKFVTVKNLDVIKKENYYEVNSKCYEEAKKLKKISNQAEFIASFYKNDL |
| | IKINGELYRVIGVNNDLLNRIEVNMIDITYREYLENMNDKRPPHIIKTIASKTQSIKKYSTDILGNLYEVKSKKHPQIIKK |
| | (SEQ ID NO: 15) |
| StCas9 | MLFNKCIIISINLDFSNKEKCMTKPYSIGLDIGTNSVGWAVITDNYKVPSKKMKVLGNTSKKYIKKNLLGVLLFDSGITAE |
| Staphylococcus | GRRLKRTARRRYTRRRNRILYLQEIFSTEMATLDDAFFQRLDDSFLVPDDKRDSKYPIFGNLVEEKVYHDEFPTIYHLRKY |
| thermophilus | LADSTKKADLRLVYLALAHMIKYRGHFLIEGEFNSKNNDIQKNFQDFLDTYNAIFESDLSLENSKQLEEIVKDKISKLEKK |
| UniProtKB/ | DRILKLFPGEKNSGIFSEFLKLIVGNQADFRKCFNLDEKASLHFSKESYDEDLETLLGYIGDDYSDVFLKAKKLYDAILLS |
| Swiss-Prot: | GFLTVTDNETEAPLSSAMIKRYNEHKEDLALLKEYIRNISLKTYNEVFKDDTKNGYAGYIDGKTNQEDFYVYLKNLLAEFE |
| G3ECR1.2 | GADYFLEKIDREDFLRKQRTFDNGSIPYQIHLQEMRAILDKQAKFYPFLAKNKERIEKILTFRIPYYVGPLARGNSDFAWS |
| Wild type | IRKRNEKITPWNFEDVIDKESSAEAFINRMTSFDLYLPEEKVLPKHSLLYETFNVYNELTKVRFIAESMRDYQFLDSKQKK |
| | DIVRLYFKDKRKVTDKDIIEYLHAIYGYDGIELKGIEKQFNSSLSTYHDLLNIINDKEFLDDSSNEAIIEEIIHTLTIFED |
| | REMIKQRLSKFENIFDKSVLKKLSRRHYTGWGKLSAKLINGIRDEKSGNTILDYLIDDGISNRNFMQLIHDDALSFKKKIQ |
| | KAQIIGDEDKGNIKEVVKSLPGSPAIKKGILQSIKIVDELVKVMGGRKPESIVVEMARENQYTNQGKSNSQQRLKRLEKSL |
| | KELGSKILKENIPAKLSKIDNNALQNDRLYLYYLQNGKDMYTGDDLDIDRLSNYDIDHIIPQAFLKDNSIDNKVLVSSASN |
| | RGKSDDFPSLEVVKKRKTFWYQLLKSKLISQRKFDNLTKAERGGLLPEDKAGFIQRQLVETRQITKHVARLLDEKFNNKKD |
| | ENNRAVRTVKIITLKSTLVSQFRKDFELYKVREINDFHHAHDAYLNAVIASALLKKYPKLEPEFVYGDYPKYNSFRERKSA |
| | TEKVYFYSNIMNIFKKSISLADGRVIERPLIEVNEETGESVWNKESDLATVRRVLSYPQVNVVKKVEEQNHGLDRGKPKGL |
| | FNANLSSKPKPNSNENLVGAKEYLDPKKYGGYAGISNSFAVLVKGTIEKGAKKKITNVLEFQGISILDRINYRKDKLNFLL |
| | EKGYKDIELIIELPKYSLFELSDGSRRMLASILSTNNKRGEIHKGNQIFLSQKFVKLLYHAKRISNTINENHRKYVENHKK |
| | EFEELFYYILEFNENYVGAKKNGKLLNSAFQSWQNHSIDELCSSFIGPTGSERKGLFELTSRGSAADFEFLGVKIPRYRDY |
| | TPSSLLKDATLIHQSVTGLYETRIDLAKLGEG(SEQ ID NO: 16) |
| LcCas9 | MKIKNYNLALTPSTSAVGHVEVDDDLNILEPVHHQKAIGVAKFGEGETAEARRLARSARRTTKRRANRINHYFNEIMKPEI |
| Lactobacillus | DKVDPLMFDRIKQAGLSPLDERKEFRTVIFDRPNIASYYHNQFPTIWHLQKYLMITDEKADIRLIYWALHSLLKHRGHFFN |
| crispatus | TTPMSQFKPGKLNLKDDMLALDDYNDLEGLSFAVANSPEIEKVIKDRSMHKKEKIAELKKLIVNDVPDKDLAKRNNKIITQ |
| NCBI | IVNAIMGNSFHLNFIFDMDLDKLTSKAWSFKLDDPELDTKFDAISGSMTDNQIGIFETLQKIYSAISLLDILNGSSNVVDA |
| Reference | KNALYDKHKRDLNLYFKFLNTLPDEIAKTLKAGYTLYIGNRKKDLLAARKLLKVNVAKNFSQDDFYKLINKELKSIDKQGL |
| Sequence: | QTRFSEKVGELVAQNNFLPVQRSSDNVFIPYQLNAITFNKILENQGKYYDFLVKPNPAKKDRKNAPYELSQLMQFTIPYYV |
| WP_133478044.1 | GPLVTPEEQVKSGIPKTSRFAWMVRKDNGAITPWNFYDKVDIEATADKFIKRSIAKDSYLLSELVLPKHSLLYEKYEVFNE |
| Wild type | LSNVSLDGKKLSGGVKQILFNEVFKKTNKVNTSRILKALAKHNIPGSKITGLSNPEEFTSSLQTYNAWKKYFPNQIDNFAY |
| | QQDLEKMIEWSTVFEDHKILAKKLDEIEWLDDDQKKFVANTRLRGWGRLSKRLLTGLKDNYGKSIMQRLETTKANFQQIVY |
| | KPEFREQIDKISQAAAKNQSLEDILANSYTSPSNRKAIRKTMSVVDEYIKLNHGKEPDKIFLMFQRSEQEKGKQTEARSKQ |
| | LNRILSQLKADKSANKLFSKQLADEFSNAIKKSKYKLNDKQYFYFQQLGRDALTGEVIDYDELYKYTVLHIIPRSKLTDDS |
| | QNNKVLTKYKIVDGSVALKFGNSYSDALGMPIKAFWTELNRLKLIPKGKLLNLTTDFSTLNKYQRDGYIARQLVETQQIVK |
| | LLATIMQSRFKHTKIIEVRNSQVANIRYQFDYFRIKNLNEYYRGFDAYLAAVVGTYLYKVYPKARRLFVYGQYLKPKKTNQ |
| | ENQDMHLDSEKKSQGFNFLWNLLYGKQDQIFVNGTDVIAFNRKDLITKMNTVYNYKSQKISLAIDYHNGAMFKATLFPRND |
| | RDTAKTRKLIPKKKDYDTDIYGGYTSNVDGYMLLAEIIKRDGNKQYGFYGVPSRLVSELDTLKKTRYTEYEEKLKEIIKPE |
| | LGVDLKKIKKIKILKNKVPFNQVIIDKGSKFFITSTSYRWNYRQLILSAESQQTLMDLVVDPDFSNHKARKDARKNADERL |
| | IKVYEEILYQVKNYMPMFVELHRCYEKLVDAQKTFKSLKISDKAMVLNQILILLHSNATSPVLEKLGYHTRFTLGKKHNLI |
| | SENAVLVTQSITGLKENHVSIKQML (SEQ ID NO: 17) |
| PdCas9 | MTNEKYSIGLDIGTSSIGFAVVNDNNRVIRVKGKNAIGVRLFDEGKAAADRRSFRTTRRSFRTTRRRLSRRRWRLKLLREI |
| Pedicoccus | FDAYITPVDEAFFIRLKESNLSPKDSKKQYSGDILFNDRSDKDFYEKYPTIYHLRNALMTEHRKFDVREIYLAIHHIMKFR |
| damnosus | GHFLNATPANNFKVGRLNLEEKFEELNDIYQRVFPDESIEFRTDNLEQIKEVLLDNKRSRADRQRTLVSDIYQSSEDKDIE |
| NCBI | KRNKAVATEILKASLGNKAKLNVITNVEVDKEAAKEWSITFDSESIDDDLAKIEGQMTDDGHEIIEVLRSLYSGITLSAIV |
| Reference | PENHTLSQSMVAKYDLHKDHLKLFKKLINGMTDTKKAKNLRAAYDGYIDGVKGKVLPQEDFYKQVQVNLDDSAEANEIQTY |
| Sequence: | IDQDIFMPKQRTKANGSIPHQLQQQELDQIIENQKAYYPWLAELNPNPDKKRQQLAKYKLDELVTFRVPYYVGPMITAKDQ |
| WP_062913273.1 | KNQSGAEFAWMIRKEPGNITPWNFDQKVDRMATANQFIKRMTTTDTYLLGEDVLPAQSLLYQKFEVLNELNKIRIDHKPIS |
| Wild type | IEQKQQIFNDLFKQFKNVTIKHLQDYLVSQGQYSKRPLIEGLADEKRFNSSLSTYSDLCGIFGAKLVEENDRQEDLEKIIE |
| | WSTIFEDKKIYRAKLNDLTWLTDDQKEKLATKRYQGWGRLSRKLLVGLKNSEHRNIMDILWITNENFMQIQAEPDFAKLVT |
| | DANKGMLEKTDSQDVINDLYTSPQNKKAIRQILLVVHDIQNAMHGQAPAKIHVEFARGEERNPRRSVQRQRQVEAAYEKVS |
| | NELVSAKVRQEFKEAINNKRDFKDRLFLYFMQGGIDIYTGKQLNIDQLSSYQIDHILPQAFVKDDSLTNRVLTNENQVKAD |
| | SVPIDIFGKKMLSVWGRMKDQGLISKGKYRNLTMNPENISAHTENGFINRQLVETRQVIKLAVNILADEYGDSTQIISVKA |
| | DLSHQMREDFELLKNRDVNDYHHAFDAYLAAFIGNYLLKRYPKLESYFVYGDFKKFTQKETKMRRFNFIYDLKHCDQVVNK |
| | ETGEILWTKDEDIKYIRHLFAYKKILVSHEVREKRGALYNQTIYKAKDDKGSGQESKKLIRIKDDKETKIYGGYSGKSLAY |
| | MTIVQITKKNKVSYRVIGIPTLALARLNKLENDSTENNGELYKIIKPQFTHYKVDKKNGEIIETTDDFKIVVSKVRFQQLI |
| | DDAGQFFMLASDTYKNNAQQLVISNNALKAINNTNITDCPRDDLERLDNLRLDSAFDEIVKKMDKYFSAYDANNFREKIRN |
| | SNLIFYQLPVEDQWENNKITELGKRTVLTRILQGLHANATTTDMSIFKIKTPFGQLRQRSGISLSENAQLIYQSPTGLFER |
| | RVQLNKIK (SEQ ID NO: 18) |
| FnCas9 | MKKQKFSDYYLGFDIGTNSVGWCVTDLDYNVLRFNKKDMWGSRLFEEAKTAAERRVQRNSRRRLKRRKWRLNLLEEIFSNE |
| Fusobaterium | ILKIDSNFFRRLKESSLWLEDKSSKEKFTLFNDDNYKDYDFYKQYPTIFHLRNELIKNPEKKDIRLVYLAIHSIFKSRGHF |
| nucleatum | LFEGQNLKEIKNFETLYNNLIAFLEDNGINKIIDKNNIEKLEKIVCDSKKGLKDKEKEFKEIFNSDKQLVAIFKLSVGSSV |
| NCBI | SLNDLFDTDEYKKGEVEKEKISFREQIYEDDKPIYYSILGEKIELLDIAKTFYDFMVLNNILADSQYISEAKVKLYEEHKK |
| Reference | DLKNLKYIIRKYNKGNYDKLFKDKNENNYSAYIGLNKEKSKKEVIEKSRLKIDDLIKNIKGYLPKVEEIEEKDKAIFNKIL |
| Sequence: | NKIELKTILPKQRISDNGTLPYQIHEAELEKILENQSKYYDFLNYEENGIITKDKLLMTFKFRIPYYVGPLNSYHKDKGGN |
| WP_060798984.1 | SWIVRKEEGKILPWNFEQKVDIEKSAEEFIKRMTNKCTYLNGEDVIPKDTFLYSEYVILNELNKVQVNDEFLNEENKRKII |
| | DELFKENKKVSEKKFKEYLLVKQIVDGTIELKGVKDSFNSNYISYIRFKDIFGEKLNLDIYKEISEKSILWKCLYGDDKKI |
| | FEKKIKNEYGDILTKDEIKKINTFKFNNWGRLSEKLLTGIEFINLETGECYSSVMDALRRTNYNLMELLSSKFTLQESINN |
| | ENKEMNEASYRDLIEESYVSPSLKRAIFQTLKIYEEIRKITGRVPKKVFIEMARGGDESMKNKKIPARQEQLKKLYDSCGN |
| | DIANFSIDIKEMKNSLISYDNNSLRQKKLYLYYLQFGKCMYTGREIDLDRLLQNNDTYDIDHIYPRSKVIKDDSFDNLVLV |
| | LKNENAEKSNEYPVKKEIQEKMKSFWRFLKEKNFISDEKYKRLTGKDDFELRGFMARQLVNVRQTTKEVGKILQQIEPEIK |
| | IVYSKAEIASSFREMFDFIKVRELNDTHHAKDAYLNIVAGNVYNTKFTEKPYRYLQEIKENYDVKKIYNYDIKNAWDKENS |
| | LEIVKKNMEKNTVNITRFIKEKKGQLFDLNPIKKGETSNEIISIKPKVYNGKDDKLNEKYGYYKSLNPAYFLYVEHKEKNK |
| | RIKSFERVNLVDVNNIKDEKSLVKYLIENKKLVEPRVIKKVYKRQVILINDYPYSIVTLDSNKLMDFENLKPLFLENKYEK |
| | ILKNVIKFLEDNQGKSEENYKFIYLKKKDRYEKNETLESVKDRYNLEFNEMYDKFLEKLDSKDYKNYMNNKKYQELLDVKE |
| | KFIKLNLFDKAFTLKSFLDLFNRKTMADFSKVGLTKYLGKIQKISSNVLSKNELYLLEESVTGLFVKKIKL (SEQ ID |
| | NO: 19) |
| EcCas9 | MNKYYLGLDMGSASVGWAVTDENYHLVRRKGKDLWGVRTFDVAQTAKERRITRGNRRRQDRRKQRIQILQELLGEEVLKTD |
| Enterococcus | PGFFHRMKESRYVVEDKRTLDGKQVELPYALFVDKDYTDKEYYKQFPTINHLIVYLMTTSDTPDIRLVYLALHYYMKNRGN |
| cecorum | FLHSGDINNVKDINDILEQLDNVLETFLDGWNLKLKSYVEDIKNIYNRDLGRGERKKAFVNTLGAKTKAEKAFCSLISGGS |
| NCBI | TNLAELFDDSSLKEIETPKIEFASSSLEDKIDGIQEALEDRFAVIEAAKRLYDWKTLTDILGDSSSLAEARVNSYQMHHEQ |
| Reference | LLELKSLVKEYLDRKVFQEVFVSLNVANNYPAYIGHTKINGKKKELEVKRTKRNDFYSYVKKQVIEPIKKKVSDEAVLTKL |
| Sequence: | SEIESLIEVDKYLPLQVNSDNGVIPYQVKLNELTRIFDNLENRIPVLRENRDKIIKTFKFRIPYYVGSLNGVVKNGKCTNW |
| WP_047338501.1 | MVRKEEGKIYPWNFEDKVDLEASAEQFIRRMTNKCTYLVNEDVLPKYSLLYSKYLVLSELNNLRIDGRPLDVKIKQDIYEN |
| Wild type | VFKKNRKVTLKKIKKYLLKEGIITDDDELSGLADDVKSSLTAYRDFKEKLGHLDLSEAQMENIILNITLFGDDKKLLKKRL |
| | AALYPFIDDKSLNRIATLNYRDWGRLSERFLSGITSVDQETGELRTIIQCMYETQANLMQLLAEPYHFVEAIEKENPKVDL |
| | ESISYRIVNDLYVSPAVKRQIWQTLLVIKDIKQVMKHDPERIFIEMAREKQESKKTKSRKQVLSEVYKKAKEYEHLFEKLN |
| | SLTEEQLRSKKIYLYFTQLGKCMYSGEPIDFENLVSANSNYDIDHIYPQSKTIDDSFNNIVLVKKSLNAYKSNHYPIDKNI |
| | RDNEKVKTLWNTLVSKGLITKEKYERLIRSTPFSDEELAGFIARQLVETRQSTKAVAEILSNWFPESEIVYSKAKNVSNFR |
| | QDFEILKVRELNDCHHAHDAYLNIVVGNAYHTKFTNSPYRFIKNKANQEYNLRKLLQKVNKIESNGVVAWVGQSENNPGTI |
| | ATVKKVIRRNTVLISRMVKEVDGQLFDLTLMKKGKGQVPIKSSDERLTDISKYGGYNKATGAYFTFVKSKKRGKVVRSFEY |
| | VPLHLSKQFENNNELLKEYIEKDRGLTDVEILIPKVLINSLFRYNGSLVRITGRGDTRLLLVHEQPLYVSNSFVQQLKSVS |
| | SYKLKKSENDNAKLTKTATEKLSNIDELYDGLLRKLDLPIYSYWFSSIKEYLVESRTKYIKLSIEEKALVIFEILHLFQSD |
| | AQVPNLKILGLSTKPSRIRIQKNLKDTDKMSIIHQSPSGIFEHEIELTSL (SEQ ID NO: 20) |
| AhCas9 | MQNGFLGITVSSEQVGWAVTNPKYELERASRKDLWGVRLFDKAETAEDRRMFRTNRRLNQRKKNRIHYLRDIFHEEVNQKD |
| Anaerostipes | PNFFQQLDESNFCEDDRTVEFNFDTNLYKNQFPTVYHLRKYLMETKDKPDIRLVYLAFSKFMKNRGHFLYKGNLGEVMDFE |
| hadrus | NSMKGFCESLEKFNIDFPTLSDEQVKEVRDILCDHKIAKTVKKKNIITITKVKSKTAKAWIGLFCGCSVPVKVLFQDIDEE |
| NCBI | IVTDPEKISFEDASYDDYIANIEKGVGIYYEAIVSAKMLFDWSILNEILGDHQLLSDAMIAEYNKHHDDLKRLQKIIKGTG |
| Reference | SRELYQDIFINDVSGNYVCYVGHAKTMSSADQKQFYTFLKNRLKNVNGISSEDAEWIDTEIKNGTLLPKQTKRDNSVIPHQ |
| Sequence: | LQLREFELILDNMQEMYPFLKENREKLLKIFNFVIPYYVGPLKGVVRKGESTNWMVPKKDGVIHPWNFDEMVDKEASAECF |
| WP_044924278.1 | ISRMTGNCSYLFNEKVLPKNSLLYETFEVLNELNPLKINGEPISVELKQRIYEQLFLTGKKVTKKSLTKYLIKNGYDKDIE |
| Wild type | LSGIDNEFHSNLKSHIDFEDYDNLSDEEVEQIILRITVFEDKQLLKDYLNREFVKLSEDERKQICSLSYKGWGNLSEMLLN |
| | GITVTDSNGVEVSVMDMLWNTNLNLMQILSKKYGYKAEIEHYNKEHEKTIYNREDLMDYLNIPPAQRRKVNQLITIVKSLK |
| | KTYGVPNKIFFKISREHQDDPKRTSSRKEQLKYLYKSLKSEDEKHLMKELDELNDHELSNDKVYLYFLQKGRCIYSGKKLN |
| | LSRLRKSNYQNDIDYIYPLSAVNDRSMNNKVLTGIQENRADKYTYFPVDSEIQKKMKGFWMELVLQGFMTKEKYFRLSREN |
| | DFSKSELVSFIEREISDNQQSGRMIASVLQYYFPESKIVFVKEKLISSFKRDFHLISSYGHNHLQAAKDAYITIVVGNVYH |
| | TKFTMDPAIYFKNHKRKDYDLNRLFLENISRDGQIAWESGPYGSIQTVRKEYAQNHIAVTKRVVEVKGGLFKQMPLKKGHG |
| | EYPLKTNDPRFGNIAQYGGYTNVTGSYFVLVESMEKGKKRISLEYVPVYLHERLEDDPGHKLLKEYLVDHRKLNHPKILLA |
| | KVRKNSLLKIDGFYYRLNGRSGNALILTNAVELIMDDWQTKTANKISGYMKRRAIDKKARVYQNEFHIQELEQLYDFYLDK |
| | LKNGVYKNRKNNQAELIHNEKEQFMELKTEDQCVLLTEIKKLFVCSPMQADLTLIGGSKHTGMIAMSSNVTKADFAVIAED |
| | PLGLRNKVIYSHKGEK (SEQ ID NO: 21) |
| KvCas9 | MSQNNNKIYNIGLDIGDASVGWAVVDEHYNLLKRHGKHMWGSRLFTQANTAVERRSSRSTRRRYNKRRERIRLLREIMEDM |
| Kandleria | VLDVDPTFFIRLANVSFLDQEDKKDYLKENYHSNYNLFIDKDFNDKTYYDKYPTIYHLRKHLCESKEKEDPRLIYLALHHI |
| vitulina | VKYRGNFLYEGQKFSMDVSNIEDKMIDVLRQFNEINLFEYVEDRKKIDEVLNVLKEPLSKKHKAEKAFALFDTTKDNKAAY |
| NCBI | KELCAALAGNKFNVTKMLKEAELHDEDEKDISFKFSDATFDDAFVEKQPLLGDCVEFIDLLHDIYSWVELQNILGSAHTSE |
| Reference | PSISAAMIQRYEDHKNDLKLLKDVIRKYLPKKYFEVFRDEKSKKNNYCNYINHPSKTPVDEFYKYIKKLIEKIDDPDVKTI |
| Sequence: | LNKIELESFMLKQNSRTNGAVPYQMQLDELNKILENQSVYYSDLKDNEDKIRSILTFRIPYYFGPLNITKDRQFDWIIKKE |
| WP_031589969.1 | GKENERILPWNANEIVDVDKTADEFIKRMRNFCTYFPDEPVMAKNSLTVSKYEVLNEINKLRINDHLIKRDMKDKMLHTLF |
| Wild type | MDHKSISANAMKKWLVKNQYFSNTDDIKIEGFQKENACSTSLTPWIDFTKIFGKINESNYDFIEKIIYDVTVFEDKKILRR |
| | RLKKEYDLDEEKIKKILKLKYSGWSRLSKKLLSGIKTKYKDSTRTPETVLEVMERTNMNLMQVINDEKLGFKKTIDDANST |
| | SVSGKFSYAEVQELAGSPAIKRGIWQALLIVDEIKKIMKHEPAHVYIEFARNEDEKERKDSFVNQMLKLYKDYDFEDETEK |
| | EANKHLKGEDAKSKIRSERLKLYYTQMGKCMYTGKSLDIDRLDTYQVDHIVPQSLLKDDSIDNKVLVLSSENQRKLDDLVI |
| | PSSIRNKMYGFWEKLFNNKIISPKKFYSLIKTEFNEKDQERFINRQIVETRQITKHVAQIIDNHYENTKVVTVRADLSHQF |
| | RERYHIYKNRDINDFHHAHDAYIATILGTYIGHRFESLDAKYIYGEYKRIFRNQKNKGKEMKKNNDGFILNSMRNIYADKD |
| | TGEIVWDPNYIDRIKKCFYYKDCFVTKKLEENNGTFFNVTVLPNDTNSDKDNTLATVPVNKYRSNVNKYGGFSGVNSFIVA |
| | IKGKKKKGKKVIEVNKLTGIPLMYKNADEEIKINYLKQAEDLEEVQIGKEILKNQLIEKDGGLYYIVAPTEIINAKQLILN |
| | ESQTKLVCEIYKAMKYKNYDNLDSEKIIDLYRLLINKMELYYPEYRKQLVKKFEDRYEQLKVISIEEKCNIIKQILATLHC |
| | NSSIGKIMYSDFKISTTIGRLNGRTISLDDISFIAESPTGMYSKKYKL (SEQ ID NO: 22) |
| EfCas9 | MRLFEEGHTAEDRRLKRTARRRISRRRNRLRYLQAFFEEAMTDLDENFFARLQESFLVPEDKKWHRHPIFAKLEDEVAYHE |
| Enterococcus | TYPTIYHLRKKLADSSEQADLRLIYLALAHIVKYRGHFLIEGKLSTENTSVKDQFQQFMVIYNQTFVNGESRLVSAPLPES |
| faecalis | VLIEEELTEKASRTKKSEKVLQQFPQEKANGLFGQFLKLMVGNKADFKKVFGLEEEAKITYASESYEEDLEGILAKVGDEY |
| NCBI | SDVFLAAKNVYDAVELSTILADSDKKSHAKLSSSMIVRFTEHQEDLKKFKRFIRENCPDEYDNLFKNEQKDGYAGYIAHAG |
| Reference | KVSQLKFYQYVKKIIQDIAGAEYFLEKIAQENFLRKQRTFDNGVIPHQIHLAELQAIIHRQAAYYPFLKENQEKIEQLVTF |
| Sequence: | RIPYYVGPLSKGDASTFAWLKRQSEEPIRPWNLQETVDLDQSATAFIERMTNFDTYLPSEKVLPKHSLLYEKFMVFNELTK |
| WP_016631044.1 | ISYTDDRGIKANFSGKEKEKIFDYLFKTRRKVKKKDIIQFYRNEYNTEIVTLSGLEEDQFNASFSTYQDLLKCGLTRAELD |
| Wild type | HPDNAEKLEDIIKILTIFEDRQRIRTQLSTFKGQFSAEVLKKLERKHYTGWGRLSKKLINGIYDKESGKTILDYLVKDDGV |
| | SKHYNRNFMQLINDSQLSFKNAIQKAQSSEHEETLSETVNELAGSPAIKKGIYQSLKIVDELVAIMGYAPKRIVVEMAREN |
| | QTTSTGKRRSIQRLKIVEKAMAEIGSNLLKEQPTTNEQLRDTRLFLYYMQNGKDMYTGDELSLHRLSHYDIDHIIPQSFMK |
| | DDSLDNLVLVGSTENRGKSDDVPSKEVVKDMKAYWEKLYAAGLISQRKFQRLTKGEQGGLTLEDKAHFIQRQLVETRQITK |
| | NVAGILDQRYNAKSKEKKVQIITLKASLTSQFRSIFGLYKVREVNDYHHGQDAYLNCVVATTLLKVYPNLAPEFVYGEYPK |
| | FQTFKENKATAKAIIYTNLLRFFTEDEPRFTKDGEILWSNSYLKTIKKELNYHQMNIVKKVEVQKGGFSKESIKPKGPSNK |
| | LIPVKNGLDPQKYGGFDSPVVAYTVLFTHEKGKKPLIKQEILGITIMEKTRFEQNPILFLEEKGFLRPRVLMKLPKYTLYE |
| | FPEGRRRLLASAKEAQKGNQMVLPEHLLTLLYHAKQCLLPNQSESLAYVEQHQPEFQEILERVVDFAEVHTLAKSKVQQIV |
| | KLFEANQTADVKEIAASFIQLMQFNAMGAPSTFKFFQKDIERARYTSIKEIFDATIIYQSPTGLYETRRKVVD (SEQ ID |
| | NO: 23) |
| Staphylococcus | KRNYILGLDIGITSVGYGIIDYETRDVIDAGVRLFKEANVENNEGRRSKRGARRLKRRRRHRIQRVKKLLFDYNLLTDHSE |
| aureus | LSGINPYEARVKGLSQKLSEEEFSAALLHLAKRRGVHNVNEVEEDTGNELSTKEQISRNSKALEEKYVAELQLERLKKDGE |
| Cas9 | VRGSINRFKTSDYVKEAKQLLKVQKAYHQLDQSFIDTYIDLLETRRTYYEGPGEGSPFGWKDIKEWYEMLMGHCTYFPEEL |
| | RSVKYAYNADLYNALNDLNNLVITRDENEKLEYYEKFQIIENVFKQKKKPTLKQIAKEILVNEEDIKGYRVTSTGKPEFTN |
| | LKVYHDIKDITARKEIIENAELLDQIAKILTIYQSSEDIQEELTNLNSELTQEEIEQISNLKGYTGTHNLSLKAINLILDE |
| | LWHTNDNQIAIFNRLKLVPKKVDLSQQKEIPTTLVDDFILSPVVKRSFIQSIKVINAIIKKYGLPNDIIIELAREKNSKDA |
| | QKMINEMQKRNRQTNERIEEIIRTTGKENAKYLIEKIKLHDMQEGKCLYSLEAIPLEDLLNNPFNYEVDHIIPRSVSFDNS |
| | FNNKVLVKQEENSKKGNRTPFQYLSSSDSKISYETFKKHILNLAKGKGRISKTKKEYLLEERDINRFSVQKDFINRNLVDT |
| | RYATRGLMNLLRSYFRVNNLDVKVKSINGGFTSFLRRKWKFKKERNKGYKHHAEDALIIANADFIFKEWKKLDKAKKVMEN |
| | QMFEEKQAESMPEIETEQEYKEIFITPHQIKHIKDFKDYKYSHRVDKKPNRELINDTLYSTRKDDKGNTLIVNNLNGLYDK |
| | DNDKLKKLINKSPEKLLMYHHDPQTYQKLKLIMEQYGDEKNPLYKYYEETGNYLTKYSKKDNGPVIKKIKYYGNKLNAHLD |
| | ITDDYPNSRNKVVKLSLKPYRFDVYLDNGVYKFVTVKNLDVIKKENYYEVNSKCYEEAKKLKKISNQAEFIASFYNNDLIK |
| | INGELYRVIGVNNDLLNRIEVNMIDITYREYLENMNDKRPPRIIKTIASKTQSIKKYSTDILGNLYEVKSKKHPQIIKKG |
| | (SEQ ID NO: 24) |
| Geobacillus | MKYKIGLDIGITSIGWAVINLDIPRIEDLGVRIFDRAENPKTGESLALPRRLARSARRRLRRRKHRLERIRRLFVREGILT |
| thermodenitrifleans | KEELNKLFEKKHEIDVWQLRVEALDRKLNNDELARILLHLAKRRGFRSNRKSERTNKENSTMLKHIEENQSILSSYRTVAE |
| Cas9 | MVVKDPKFSLHKRNKEDNYTNTVARDDLEREIKLIFAKQREYGNIVCTEAFEHEYISIWASQRPFASKDDIEKKVGFCTFE |
| | PKEKRAPKATYTFQSFTVWEHINKLRLVSPGGIRALTDDERRLIYKQAFHKNKITFHDVRTLLNLPDDTRFKGLLYDRNTT |
| | LKENEKVRFLELGAYHKIRKAIDSVYGKGAAKSFRPIDFDTFGYALTMFKDDTDIRSYLRNEYEQNGKRMENLADKVYDEE |
| | LIEELLNLSFSKFGHLSLKALRNILPYMEQGEVYSTACERAGYTFTGPKKKQKTVLLPNIPPIANPVVMRALTQARKVVNA |
| | IIKKYGSPVSIHIELARELSQSFDERRKMQKEQEGNRKKNETAIRQLVEYGLTLNPTGLDIVKFKLWSEQNGKCAYSLQPI |
| | EIERLLEPGYTEVDHVIPYSRSLDDSYTNKVLVLTKENREKGNRTPAEYLGLGSERWQQFETFVLTNKQFSKKKRDRLLRL |
| | HYDENEENEFKNRNLNDTRYISRFLANFIREHLKFADSDDKQKVYTVNGRITAHLRSRWNFNKNREESNLHHAVDAAIVAC |
| | TTPSDIARVTAFYQRREQNKELSKKTDPQFPQPWPHFADELQARLSKNPKESIKALNLGNYDNEKLESLQPVFVSRMPKRS |
| | ITGAAHQETLRRYIGIDERSGKIQTVVKKKLSEIQLDKTGHFPMYGKESDPRTYEAIRQRLLEHNNDPKKAFQEPLYKPKK |
| | NGELGPIIRTIKIIDTTNQVIPLNDGKTVAYNSNIVRVDVFEKDGKYYCVPIYTIDMMKGILPNKAIEPNKPYSEWKEMTE |
| | DYTFRFSLYPNDLIRIEFPREKTIKTAVGEEIKIKDLFAYYQTIDSSNGGLSLVSHDNNFSLRSIGSRTLKRFEKYQVDVL |
| | GNIYKVRGEKRVGVASSSHSKAGETIRPL |
| | (SEQ ID NO: 25) |
| ScCas9 | MEKKYSIGLDIGTNSVGWAVITDDYKVPSKKFKVLGNTNRKSIKKNLMGALLFDSGETAEATRLKRTARRRYTRRKNRIRY |
| S. canis | LQEIFANEMAKLDDSFFQRLEESFLVEEDKKNERHPIFGNLADEVAYHRNYPTIYHLRKKLADSPEKADLRLIYLALAHII |
| 1375 AA | KFRGHFLIEGKLNAENSDVAKLFYQLIQTYNQLFEESPLDEIEVDAKGILSARLSKSKRLEKLIAVFPNEKKNGLFGNIIA |
| 159.2 kDa | LALGLTPNFKSNFDLTEDAKLQLSKDTYDDDLDELLGQIGDQYADLFSAAKNLSDAILLSDILRSNSEVTKAPLSASMVKR |
| | YDEHHQDLALLKTLVRQQFPEKYAEIFKDDTKNGYAGYVGIGIKHRKRTTKLATQEEFYKFIKPILEKMDGAEELLAKLNR |
| | DDLLRKQRTFDNGSIPHQIHLKELHAILRRQEEFYPFLKENREKIEKILTFRIPYYVGPLARGNSRFAWLTRKSEEAITPW |
| | NFEEVVDKGASAQSFIERMTNFDEQLPNKKVLPKHSLLYEYFTVYNELTKVKYVTERMRKPEFLSGEQKKAIVDLLFKTNR |
| | KVTVKQLKEDYFKKIECFDSVEIIGVEDRFNASLGTYHDLLKIIKDKDFLDNEENEDILEDIVLTLTLFEDREMIEERLKT |
| | YAHLFDDKVMKQLKRRHYTGWGRLSRKMINGIRDKQSGKTILDFLKSDGFSNRNFMQLIHDDSLTFKEEIEKAQVSGQGDS |
| | LHEQIADLAGSPAIKKGILQTVKIVDELVKVMGHKPENIVIEMARENQTTTKGLQQSRERKKRIEEGIKELESQILKENPV |
| | ENTQLQNEKLYLYYLQNGRDMYVDQELDINRLSDYDVDHIVPQSFIKDDSIDNKVLTRSVENRGKSDNVPSEEVVKKMKNY |
| | WRQLLNAKLITQRKFDNLTKAERGGLSEADKAGFIKRQLVETRQITKHVARILDSRMNTKRDKNDKPIREVKVITLKSKLV |
| | SDFRKDFQLYKVRDINNYHHAHDAYLNAVVGTALIKKYPKLESEFVYGDYKVYDVRKMIAKSEQEIGKATAKRFFYSNIMN |
| | FFKTEVKLANGEIRKRPLIETNGETGEVVWNKEKDFATVRKVLAMPQVNIVKKTEVQTGGFSKESILSKRESAKLIPRKKG |
| | WDTRKYGGFGSPTVAYSILVVAKVEKGKAKKLKSVKVLVGITIMEKGSYEKDPIGFLEAKGYKDIKKELIFKLPKYSLFEL |
| | ENGRRRMLASATELQKANELVLPQHLVRLLYYTQNISATTGSNNLGYIEQHREEFKEIFEKIIDFSEKYILKNKVNSNLKS |
| | SFDEQFAVSDSILLSNSFVSLLKYTSFGASGGFTFLDLDVKQGRLRYQTVTEVLDATLIYQSITGLYETRTDLSQLGGD |
| | (SEQ ID NO: 26) |

The base editors described herein may include any of the above Cas9 ortholog sequences, or any variants thereof having at least 80%, at least 85%, at least 90%, at least 95%, or at least 99% sequence identity thereto.

The napDNAbp may include any suitable homologs and/or orthologs or naturally occurring enzymes, such as, Cas9. Cas9 homologs and/or orthologs have been described in various species, including, but not limited to, S. pyogenes and S. thermophilus. Preferably, the Cas moiety is configured (e.g., mutagenized, recombinantly engineered, or otherwise obtained from nature) as a nickase, i.e., capable of cleaving only a single strand of the target doubpdditional suitable Cas9 nucleases and sequences will be apparent to those of skill in the art based on this disclosure, and such Cas9 nucleases and sequences include Cas9 sequences from the organisms and loci disclosed in Chylinski, Rhun, and Charpentier, “The tracrRNA and Cas9 families of type II CRISPR-Cas immunity systems” (2013) RNA Biology 10:5, 726-737; the entire contents of which are incorporated herein by reference. In some embodiments, a Cas9 nuclease has an inactive (e.g., an inactivated) DNA cleavage domain, that is, the Cas9 is a nickase. In some embodiments, the Cas9 protein comprises an amino acid sequence that is at least 80% identical to the amino acid sequence of a Cas9 protein as provided by any one of the variants of Table 3. In some embodiments, the Cas9 protein comprises an amino acid sequence that is at least 85%, at least 90%, at least 92%, at least 95%, at least 96%, at least 97%, at least 98%, at least 99%, or at least 99.5% identical to the amino acid sequence of a Cas9 protein as provided by any one of the Cas9 orthologs in the above tables.

(3) Dead Cas9 Variant

In certain embodiments, the base editors described herein may include a dead Cas9, e.g., dead SpCas9, which has no nuclease activity due to one or more mutations that inactive both nuclease domains of Cas9, namely the RuvC domain (which cleaves the non-protospacer DNA strand) and HNH domain (which cleaves the protospacer DNA strand). The nuclease inactivation may be due to one or mutations that result in one or more substitutions and/or deletions in the amino acid sequence of the encoded protein, or any variants thereof having at least 80%, at least 85%, at least 90%, at least 95%, or at least 99% sequence identity thereto.

As used herein, the term “dCas9” refers to a nuclease-inactive Cas9 or nuclease-dead Cas9, or a functional fragment thereof, and embraces any naturally occurring dCas9 from any organism, any naturally-occurring dCas9 equivalent or functional fragment thereof, any dCas9 homolog, ortholog, or paralog from any organism, and any mutant or variant of a dCas9, naturally-occurring or engineered. The term dCas9 is not meant to be particularly limiting and may be referred to as a “dCas9 or equivalent.” Exemplary dCas9 proteins and method for making dCas9 proteins are further described herein and/or are described in the art and are incorporated herein by reference.

In other embodiments, dCas9 corresponds to, or comprises in part or in whole, a Cas9 amino acid sequence having one or more mutations that inactivate the Cas9 nuclease activity. In other embodiments, Cas9 variants having mutations other than D10A and H840A are provided which may result in the full or partial inactivate of the endogenous Cas9 nuclease activity (e.g., nCas9 or dCas9, respectively). Such mutations, by way of example, include other amino acid substitutions at D10 and H820, or other substitutions within the nuclease domains of Cas9 (e.g., substitutions in the HNH nuclease subdomain and/or the RuvC1 subdomain) with reference to a wild type sequence such as Cas9 from Streptococcus pyogenes (NCBI Reference Sequence: NC_017053.1). In some embodiments, variants or homologues of Cas9 (e.g., variants of Cas9 from Streptococcus pyogenes (NCBI Reference Sequence: NC_017053.1)) are provided which are at least about 70% identical, at least about 80% identical, at least about 90% identical, at least about 95% identical, at least about 98% identical, at least about 99% identical, at least about 99.5% identical, or at least about 99.9% identical to NCBI Reference Sequence: NC_017053.1. In some embodiments, variants of dCas9 (e.g., variants of NCBI Reference Sequence: NC_017053.1) are provided having amino acid sequences which are shorter, or longer than NC_017053.1 by about 5 amino acids, by about 10 amino acids, by about 15 amino acids, by about 20 amino acids, by about 25 amino acids, by about 30 amino acids, by about 40 amino acids, by about 50 amino acids, by about 75 amino acids, by about 100 amino acids or more.

In one embodiment, the dead Cas9 may be based on the canonical SpCas9 sequence of Q99ZW2 and may have the following sequence, which comprises a D10A and an H810A substitutions (underlined and bolded), or a variant be variant of SEQ ID NO: 27 having at least 80%, at least 85%, at least 90%, at least 95%, or at least 99% sequence identity thereto:

| Description | Sequence | SEQ ID NO: |
|---|---|---|
| dead Cas9 or | MDKKYSIGL [symbol] IGTNSVGWAVITDEYKVPSKKFKVLGNTDRHSIKKNLIGALLFDSGETA | 27 |
| dCas9 | EATRLKRTARRRYTRRKNRICYLQEIFSNEMAKVDDSFFHRLEESFLVEEDKKHERHPI | |
| Streptococcus | FGNIVDEVAYHEKYPTIYHLRKKLVDSTDKADLRLIYLALAHMIKFRGHFLIEGDLNPD | |
| pyogenes | NSDVDKLFIQLVQTYNQLFEENPINASGVDAKAILSARLSKSRRLENLIAQLPGEKKNG | |
| Q992W2 Cas9 | LFGNLIALSLGLTPNFKSNFDLAEDAKLQLSKDTYDDDLDNLLAQIGDQYADLFLAAKN | |
| with D10X | LSDAILLSDILRVNTEITKAPLSASMIKRYDEHHQDLTLLKALVRQQLPEKYKEIFFDQ | |
| and H810 [symbol] | SKNGYAGYIDGGASQEEFYKFIKPILEKMDGTEELLVKLNREDLLRKQRTFDNGSIPHQ | |
| Where “X” is | IHLGELHAILRRQEDFYPFLKDNREKIEKILTFRIPYYVGPLARGNSRFAWMTRKSEET | |
| any amino | ITPWNFEEVVDKGASAQSFIERMTNFDKNLPNEKVLPKHSLLYEYFTVYNELTKVKYVT | |
| acid | EGMRKPAFLSGEQKKAIVDLLFKTNRKVTVKQLKEDYFKKIECFDSVEISGVEDRFNAS | |
| | LGTYHDLLKIIKDKDFLDNEENEDILEDIVLTLTLFEDREMIEERLKTYAHLFDDKVMK | |
| | QLKRRRYTGWGRLSRKLINGIRDKQSGKTILDFLKSDGFANRNFMQLIHDDSLTFKEDI | |
| | QKAQVSGQGDSLHEHIANLAGSPAIKKGILQTVKVVDELVKVMGRHKPENIVIEMAREN | |
| | QTTQKGQKNSRERMKRIEEGIKELGSQILKEHPVENTQLQNEKLYLYYLQNGRDMYVDQ | |
| | ELDINRLSDYDVD [symbol] IVPQSFLKDDSIDNKVLTRSDKNRGKSDNVPSEEVVKKMKNYWRQ | |
| | LLNAKLITQRKFDNLTKAERGGLSELDKAGFIKRQLVETRQITKHVAQILDSRMNTKYD | |
| | ENDKLIREVKVITLKSKLVSDFRKDFQFYKVREINNYHHAHDAYLNAVVGTALIKKYPK | |
| | LESEFVYGDYKVYDVRKMIAKSEQEIGKATAKYFFYSNIMNFFKTEITLANGEIRKRPL | |
| | IETNGETGEIVWDKGRDFATVRKVLSMPQVNIVKKTEVQTGGFSKESILPKRNSDKLIA | |
| | RKKDWDPKKYGGFDSPTVAYSVLVVAKVEKGKSKKLKSVKELLGITIMERSSFEKNPID | |
| | FLEAKGYKEVKKDLIIKLPKYSLFELENGRKRMLASAGELQKGNELALPSKYVNFLYLA | |
| | SHYEKLKGSPEDNEQKQLFVEQHKHYLDEIIEQISEFSKRVILADANLDKVLSAYNKHR | |
| | DKPIREQAENIIHLFTLTNLGAPAAFKYFDTTIDRKRYTSTKEVLDATLIHQSITGLYE | |
| | TRIDLSQLGGD | |
| dead Cas9 or | MDKKYSIGL [symbol] IGTNSVGWAVITDEYKVPSKKFKVLGNTDRHSIKKNLIGALLFDSGETA | 28 |
| dCas9 | EATRLKRTARRRYTRRKNRICYLQEIFSNEMAKVDDSFFHRLEESFLVEEDKKHERHPI | |
| Streptococcus | FGNIVDEVAYHEKYPTIYHLRKKLVDSTDKADLRLIYLALAHMIKFRGHFLIEGDLNPD | |
| pyogenes | NSDVDKLFIQLVQTYNQLFEENPINASGVDAKAILSARLSKSRRLENLIAQLPGEKKNG | |
| Q992W2 Cas9 | LFGNLIALSLGLTPNFKSNFDLAEDAKLQLSKDTYDDDLDNLLAQIGDQYADLFLAAKN | |
| with D10 [symbol] | LSDAILLSDILRVNTEITKAPLSASMIKRYDEHHQDLTLLKALVRQQLPEKYKEIFFDQ | |
| and H810 [symbol] | SKNGYAGYIDGGASQEEFYKFIKPILEKMDGTEELLVKLNREDLLRKQRTFDNGSIPHQ | |
| | IHLGELHAILRRQEDFYPFLKDNREKIEKILTFRIPYYVGPLARGNSRFAWMTRKSEET | |
| | ITPWNFEEVVDKGASAQSFIERMTNFDKNLPNEKVLPKHSLLYEYFTVYNELTKVKYVT | |
| | EGMRKPAFLSGEQKKAIVDLLFKTNRKVTVKQLKEDYFKKIECFDSVEISGVEDRFNAS | |
| | LGTYHDLLKIIKDKDFLDNEENEDILEDIVLTLTLFEDREMIEERLKTYAHLFDDKVMK | |
| | QLKRRRYTGWGRLSRKLINGIRDKQSGKTILDFLKSDGFANRNFMQLIHDDSLTFKEDI | |
| | QKAQVSGQGDSLHEHIANLAGSPAIKKGILQTVKVVDELVKVMGRHKPENIVIEMAREN | |
| | QTTQKGQKNSRERMKRIEEGIKELGSQILKEHPVENTQLQNEKLYLYYLQNGRDMYVDQ | |
| | ELDINRLSDYDVD [symbol] IVPQSFLKDDSIDNKVLTRSDKNRGKSDNVPSEEVVKKMKNYWRQ | |
| | LLNAKLITQRKFDNLTKAERGGLSELDKAGFIKRQLVETRQITKHVAQILDSRMNTKYD | |
| | ENDKLIREVKVITLKSKLVSDFRKDFQFYKVREINNYHHAHDAYLNAVVGTALIKKYPK | |
| | LESEFVYGDYKVYDVRKMIAKSEQEIGKATAKYFFYSNIMNFFKTEITLANGEIRKRPL | |
| | IETNGETGEIVWDKGRDFATVRKVLSMPQVNIVKKTEVQTGGFSKESILPKRNSDKLIA | |
| | RKKDWDPKKYGGFDSPTVAYSVLVVAKVEKGKSKKLKSVKELLGITIMERSSFEKNPID | |
| | FLEAKGYKEVKKDLIIKLPKYSLFELENGRKRMLASAGELQKGNELALPSKYVNFLYLA | |
| | SHYEKLKGSPEDNEQKQLFVEQHKHYLDEIIEQISEFSKRVILADANLDKVLSAYNKHR | |
| | DKPIREQAENIIHLFTLTNLGAPAAFKYFDTTIDRKRYTSTKEVLDATLIHQSITGLYE | |
| | TRIDLSQLGGD | |

(4) Cas9 Nickase Variant

In one embodiment, the base editors described herein comprise a Cas9 nickase. The term “Cas9 nickase” of “nCas9” refers to a variant of Cas9 which is capable of introducing a single-strand break in a double strand DNA molecule target. In some embodiments, the Cas9 nickase comprises only a single functioning nuclease domain. The wild type Cas9 (e.g., the canonical SpCas9) comprises two separate nuclease domains, namely, the RuvC domain (which cleaves the non-protospacer DNA strand) and HNH domain (which cleaves the protospacer DNA strand). In one embodiment, the Cas9 nickase comprises a mutation in the RuvC domain which inactivates the RuvC nuclease activity. For example, mutations in aspartate (D) 10, histidine (H) 983, aspartate (D) 986, or glutamate (E) 762, have been reported as loss-of-function mutations of the RuvC nuclease domain and the creation of a functional Cas9 nickase (e.g., Nishimasu et al., “Crystal structure of Cas9 in complex with guide RNA and target DNA,” Cell 156(5), 935-949, which is incorporated herein by reference). Thus, nickase mutations in the RuvC domain could include D10X, H983X, D986X, or E762X, wherein X is any amino acid other than the wild type amino acid. In certain embodiments, the nickase could be D10A, of H983A, or D986A, or E762A, or a combination thereof.

In various embodiments, the Cas9 nickase can having a mutation in the RuvC nuclease domain and have one of the following amino acid sequences, or a variant thereof having an amino acid sequence that has at least 80%, at least 85%, at least 90%, at least 95%, or at least 99% sequence identity thereto.

| Description | Sequence | SEQ ID NO: |
|---|---|---|
| Cas9 nickase | MDKKYSIGL [symbol] IGTNSVGWAVITDEYKVPSKKFKVLGNTDRHSIKKNLIGALLFDSGETA | 29 |
| Streptococcus | EATRLKRTARRRYTRRKNRICYLQEIFSNEMAKVDDSFFHRLEESFLVEEDKKHERHPI | |
| pyogenes | FGNIVDEVAYHEKYPTIYHLRKKLVDSTDKADLRLIYLALAHMIKFRGHFLIEGDLNPD | |
| Q99ZW2 Cas9 | NSDVDKLFIQLVQTYNQLFEENPINASGVDAKAILSARLSKSRRLENLIAQLPGEKKNG | |
| with D10 [symbol] , | LFGNLIALSLGLTPNFKSNFDLAEDAKLQLSKDTYDDDLDNLLAQIGDQYADLFLAAKN | |
| wherein X is | LSDAILLSDILRVNTEITKAPLSASMIKRYDEHHQDLTLLKALVRQQLPEKYKEIFFDQ | |
| any | SKNGYAGYIDGGASQEEFYKFIKPILEKMDGTEELLVKLNREDLLRKQRTFDNGSIPHQ | |
| alternate | IHLGELHAILRRQEDFYPFLKDNREKIEKILTFRIPYYVGPLARGNSRFAWMTRKSEET | |
| amino acid | ITPWNFEEVVDKGASAQSFIERMTNFDKNLPNEKVLPKHSLLYEYFTVYNELTKVKYVT | |
| | EGMRKPAFLSGEQKKAIVDLLFKTNRKVTVKQLKEDYFKKIECFDSVEISGVEDRFNAS | |
| | LGTYHDLLKIIKDKDFLDNEENEDILEDIVLTLTLFEDREMIEERLKTYAHLFDDKVMK | |
| | QLKRRRYTGWGRLSRKLINGIRDKQSGKTILDFLKSDGFANRNFMQLIHDDSLTFKEDI | |
| | QKAQVSGQGDSLHEHIANLAGSPAIKKGILQTVKVVDELVKVMGRHKPENIVIEMAREN | |
| | QTTQKGQKNSRERMKRIEEGIKELGSQILKEHPVENTQLQNEKLYLYYLQNGRDMYVDQ | |
| | ELDINRLSDYDVDHIVPQSFLKDDSIDNKVLTRSDKNRGKSDNVPSEEVVKKMKNYWRQ | |
| | LLNAKLITQRKFDNLTKAERGGLSELDKAGFIKRQLVETRQITKHVAQILDSRMNTKYD | |
| | ENDKLIREVKVITLKSKLVSDFRKDFQFYKVREINNYHHAHDAYLNAVVGTALIKKYPK | |
| | LESEFVYGDYKVYDVRKMIAKSEQEIGKATAKYFFYSNIMNFFKTEITLANGEIRKRPL | |
| | IETNGETGEIVWDKGRDFATVRKVLSMPQVNIVKKTEVQTGGFSKESILPKRNSDKLIA | |
| | RKKDWDPKKYGGFDSPTVAYSVLVVAKVEKGKSKKLKSVKELLGITIMERSSFEKNPID | |
| | FLEAKGYKEVKKDLIIKLPKYSLFELENGRKRMLASAGELQKGNELALPSKYVNFLYLA | |
| | SHYEKLKGSPEDNEQKQLFVEQHKHYLDEIIEQISEFSKRVILADANLDKVLSAYNKHR | |
| | DKPIREQAENIIHLFTLTNLGAPAAFKYFDTTIDRKRYTSTKEVLDATLIHQSITGLYE | |
| | TRIDLSQLGGD | |
| Cas9 nickase | MDKKYSIGLDIGTNSVGWAVITDEYKVPSKKFKVLGNTDRHSIKKNLIGALLFDSGETA | 30 |
| Streptococcus | EATRLKRTARRRYTRRKNRICYLQEIFSNEMAKVDDSFFHRLEESFLVEEDKKHERHPI | |
| pyogenes | FGNIVDEVAYHEKYPTIYHLRKKLVDSTDKADLRLIYLALAHMIKFRGHFLIEGDLNPD | |
| Q99ZW2 Cas9 | NSDVDKLFIQLVQTYNQLFEENPINASGVDAKAILSARLSKSRRLENLIAQLPGEKKNG | |
| with E762X, | LFGNLIALSLGLTPNFKSNFDLAEDAKLQLSKDTYDDDLDNLLAQIGDQYADLFLAAKN | |
| wherein X is | LSDAILLSDILRVNTEITKAPLSASMIKRYDEHHQDLTLLKALVRQQLPEKYKEIFFDQ | |
| any | SKNGYAGYIDGGASQEEFYKFIKPILEKMDGTEELLVKLNREDLLRKQRTFDNGSIPHQ | |
| alternate | IHLGELHAILRRQEDFYPFLKDNREKIEKILTFRIPYYVGPLARGNSRFAWMTRKSEET | |
| amino acid | ITPWNFEEVVDKGASAQSFIERMTNFDKNLPNEKVLPKHSLLYEYFTVYNELTKVKYVT | |
| | EGMRKPAFLSGEQKKAIVDLLFKTNRKVTVKQLKEDYFKKIECFDSVEISGVEDRFNAS | |
| | LGTYHDLLKIIKDKDFLDNEENEDILEDIVLTLTLFEDREMIEERLKTYAHLFDDKVMK | |
| | QLKRRRYTGWGRLSRKLINGIRDKQSGKTILDFLKSDGFANRNFMQLIHDDSLTFKEDI | |
| | QKAQVSGQGDSLHEHIANLAGSPAIKKGILQTVKVVDELVKVMGRHKPENIVI [symbol] MAREN | |
| | QTTQKGQKNSRERMKRIEEGIKELGSQILKEHPVENTQLQNEKLYLYYLQNGRDMYVDQ | |
| | ELDINRLSDYDVDHIVPQSFLKDDSIDNKVLTRSDKNRGKSDNVPSEEVVKKMKNYWRQ | |
| | LLNAKLITQRKFDNLTKAERGGLSELDKAGFIKRQLVETRQITKHVAQILDSRMNTKYD | |
| | ENDKLIREVKVITLKSKLVSDFRKDFQFYKVREINNYHHAHDAYLNAVVGTALIKKYPK | |
| | LESEFVYGDYKVYDVRKMIAKSEQEIGKATAKYFFYSNIMNFFKTEITLANGEIRKRPL | |
| | IETNGETGEIVWDKGRDFATVRKVLSMPQVNIVKKTEVQTGGFSKESILPKRNSDKLIA | |
| | RKKDWDPKKYGGFDSPTVAYSVLVVAKVEKGKSKKLKSVKELLGITIMERSSFEKNPID | |
| | FLEAKGYKEVKKDLIIKLPKYSLFELENGRKRMLASAGELQKGNELALPSKYVNFLYLA | |
| | SHYEKLKGSPEDNEQKQLFVEQHKHYLDEIIEQISEFSKRVILADANLDKVLSAYNKHR | |
| | DKPIREQAENIIHLFTLTNLGAPAAFKYFDTTIDRKRYTSTKEVLDATLIHQSITGLYE | |
| | TRIDLSQLGGD | |
| Cas9 nickase | MDKKYSIGLDIGTNSVGWAVITDEYKVPSKKFKVLGNTDRHSIKKNLIGALLFDSGETA | 31 |
| Streptococcus | EATRLKRTARRRYTRRKNRICYLQEIFSNEMAKVDDSFFHRLEESFLVEEDKKHERHPI | |
| pyogenes | FGNIVDEVAYHEKYPTIYHLRKKLVDSTDKADLRLIYLALAHMIKFRGHFLIEGDLNPD | |
| Q992W2 Cas9 | NSDVDKLFIQLVQTYNQLFEENPINASGVDAKAILSARLSKSRRLENLIAQLPGEKKNG | |
| with H983X, | LFGNLIALSLGLTPNFKSNFDLAEDAKLQLSKDTYDDDLDNLLAQIGDQYADLFLAAKN | |
| wherein X is | LSDAILLSDILRVNTEITKAPLSASMIKRYDEHHQDLTLLKALVRQQLPEKYKEIFFDQ | |
| any | SKNGYAGYIDGGASQEEFYKFIKPILEKMDGTEELLVKLNREDLLRKQRTFDNGSIPHQ | |
| alternate | IHLGELHAILRRQEDFYPFLKDNREKIEKILTFRIPYYVGPLARGNSRFAWMTRKSEET | |
| amino acid | ITPWNFEEVVDKGASAQSFIERMTNFDKNLPNEKVLPKHSLLYEYFTVYNELTKVKYVT | |
| | EGMRKPAFLSGEQKKAIVDLLFKTNRKVTVKQLKEDYFKKIECFDSVEISGVEDRFNAS | |
| | LGTYHDLLKIIKDKDFLDNEENEDILEDIVLTLTLFEDREMIEERLKTYAHLFDDKVMK | |
| | QLKRRRYTGWGRLSRKLINGIRDKQSGKTILDFLKSDGFANRNFMQLIHDDSLTFKEDI | |
| | QKAQVSGQGDSLHEHIANLAGSPAIKKGILQTVKVVDELVKVMGRHKPENIVIEMAREN | |
| | QTTQKGQKNSRERMKRIEEGIKELGSQILKEHPVENTQLQNEKLYLYYLQNGRDMYVDQ | |
| | ELDINRLSDYDVDHIVPQSFLKDDSIDNKVLTRSDKNRGKSDNVPSEEVVKKMKNYWRQ | |
| | LLNAKLITQRKFDNLTKAERGGLSELDKAGFIKRQLVETRQITKHVAQILDSRMNTKYD | |
| | ENDKLIREVKVITLKSKLVSDFRKDFQFYKVREINNYH [symbol] AHDAYLNAVVGTALIKKYPK | |
| | LESEFVYGDYKVYDVRKMIAKSEQEIGKATAKYFFYSNTMNFFKTEITLANGEIRKRPL | |
| | IETNGETGEIVWDKGRDFATVRKVLSMPQVNIVKKTEVQTGGFSKESILPKRNSDKLIA | |
| | RKKDWDPKKYGGFDSPTVAYSVLVVAKVEKGKSKKLKSVKELLGITIMERSSFEKNPID | |
| | FLEAKGYKEVKKDLIIKLPKYSLFELENGRKRMLASAGELQKGNELALPSKYVNFLYLA | |
| | SHYEKLKGSPEDNEQKQLFVEQHKHYLDEIIEQISEFSKRVILADANLDKVLSAYNKHR | |
| | DKPIREQAENIIHLFTLTNLGAPAAFKYFDTTIDRKRYTSTKEVLDATLIHQSITGLYE | |
| | TRIDLSQLGGD | |
| Cas9 nickase | MDKKYSIGLDIGTNSVGWAVITDEYKVPSKKFKVLGNTDRHSIKKNLIGALLFDSGETA | 32 |
| Streptococcus | EATRLKRTARRRYTRRKNRICYLQEIFSNEMAKVDDSFFHRLEESFLVEEDKKHERHPI | |
| pyogenes | FGNIVDEVAYHEKYPTIYHLRKKLVDSTDKADLRLIYLALAHMIKFRGHFLIEGDLNPD | |
| Q992W2 Cas9 | NSDVDKLFIQLVQTYNQLFEENPINASGVDAKAILSARLSKSRRLENLIAQLPGEKKNG | |
| with D986X, | LFGNLIALSLGLTPNFKSNFDLAEDAKLQLSKDTYDDDLDNLLAQIGDQYADLFLAAKN | |
| wherein X is | LSDAILLSDILRVNTEITKAPLSASMIKRYDEHHQDLTLLKALVRQQLPEKYKEIFFDQ | |
| any | SKNGYAGYIDGGASQEEFYKFIKPILEKMDGTEELLVKLNREDLLRKQRTFDNGSIPHQ | |
| alternate | IHLGELHAILRRQEDFYPFLKDNREKIEKILTFRIPYYVGPLARGNSRFAWMTRKSEET | |
| amino acid | ITPWNFEEVVDKGASAQSFIERMTNFDKNLPNEKVLPKHSLLYEYFTVYNELTKVKYVT | |
| | EGMRKPAFLSGEQKKAIVDLLFKTNRKVTVKQLKEDYFKKIECFDSVEISGVEDRFNAS | |
| | LGTYHDLLKIIKDKDFLDNEENEDILEDIVLTLTLFEDREMIEERLKTYAHLFDDKVMK | |
| | QLKRRRYTGWGRLSRKLINGIRDKQSGKTILDFLKSDGFANRNFMQLIHDDSLTFKEDI | |
| | QKAQVSGQGDSLHEHIANLAGSPAIKKGILQTVKVVDELVKVMGRHKPENIVIEMAREN | |
| | QTTQKGQKNSRERMKRIEEGIKELGSQILKEHPVENTQLQNEKLYLYYLQNGRDMYVDQ | |
| | ELDINRLSDYDVDHIVPQSFLKDDSIDNKVLTRSDKNRGKSDNVPSEEVVKKMKNYWRQ | |
| | LLNAKLITQRKFDNLTKAERGGLSELDKAGFIKRQLVETRQITKHVAQILDSRMNTKYD | |
| | ENDKLIREVKVITLKSKLVSDFRKDFQFYKVREINNYHHAH [symbol] AYLNAVVGTALIKKYPK | |
| | LESEFVYGDYKVYDVRKMIAKSEQEIGKATAKYFFYSNIMNFFKTEITLANGEIRKRPL | |
| | IETNGETGEIVWDKGRDFATVRKVLSMPQVNIVKKTEVQTGGFSKESILPKRNSDKLIA | |
| | RKKDWDPKKYGGFDSPTVAYSVLVVAKVEKGKSKKLKSVKELLGITIMERSSFEKNPID | |
| | FLEAKGYKEVKKDLIIKLPKYSLFELENGRKRMLASAGELQKGNELALPSKYVNFLYLA | |
| | SHYEKLKGSPEDNEQKQLFVEQHKHYLDEIIEQISEFSKRVILADANLDKVLSAYNKHR | |
| | DKPIREQAENIIHLFTLTNLGAPAAFKYFDTTIDRKRYTSTKEVLDATLIHQSITGLYE | |
| | TRIDLSQLGGD | |
| Cas9 nickase | MDKKYSIGL [symbol] IGTNSVGWAVITDEYKVPSKKFKVLGNTDRHSIKKNLIGALLFDSGETA | 33 |
| Streptococcus | EATRLKRTARRRYTRRKNRICYLQEIFSNEMAKVDDSFFHRLEESFLVEEDKKHERHPI | |
| pyogenes | FGNIVDEVAYHEKYPTIYHLRKKLVDSTDKADLRLIYLALAHMIKFRGHFLIEGDLNPD | |
| Q992W2 Cas9 | NSDVDKLFIQLVQTYNQLFEENPINASGVDAKAILSARLSKSRRLENLIAQLPGEKKNG | |
| with D10 [symbol] | LFGNLIALSLGLTPNFKSNFDLAEDAKLQLSKDTYDDDLDNLLAQIGDQYADLFLAAKN | |
| | LSDAILLSDILRVNTEITKAPLSASMIKRYDEHHQDLTLLKALVRQQLPEKYKEIFFDQ | |
| | SKNGYAGYIDGGASQEEFYKFIKPILEKMDGTEELLVKLNREDLLRKQRTFDNGSIPHQ | |
| | IHLGELHAILRRQEDFYPFLKDNREKIEKILTFRIPYYVGPLARGNSRFAWMTRKSEET | |
| | ITPWNFEEVVDKGASAQSFIERMTNFDKNLPNEKVLPKHSLLYEYFTVYNELTKVKYVT | |
| | EGMRKPAFLSGEQKKAIVDLLFKTNRKVTVKQLKEDYFKKIECFDSVEISGVEDRFNAS | |
| | LGTYHDLLKIIKDKDFLDNEENEDILEDIVLTLTLFEDREMIEERLKTYAHLFDDKVMK | |
| | QLKRRRYTGWGRLSRKLINGIRDKQSGKTILDFLKSDGFANRNFMQLIHDDSLTFKEDI | |
| | QKAQVSGQGDSLHEHIANLAGSPAIKKGILQTVKVVDELVKVMGRHKPENIVIEMAREN | |
| | QTTQKGQKNSRERMKRIEEGIKELGSQILKEHPVENTQLQNEKLYLYYLQNGRDMYVDQ | |
| | ELDINRLSDYDVDHIVPQSFLKDDSIDNKVLTRSDKNRGKSDNVPSEEVVKKMKNYWRQ | |
| | LLNAKLITQRKFDNLTKAERGGLSELDKAGFIKRQLVETRQITKHVAQILDSRMNTKYD | |
| | ENDKLIREVKVITLKSKLVSDFRKDFQFYKVREINNYHHAHDAYLNAVVGTALIKKYPK | |
| | LESEFVYGDYKVYDVRKMIAKSEQEIGKATAKYFFYSNIMNFFKTEITLANGEIRKRPL | |
| | IETNGETGEIVWDKGRDFATVRKVLSMPQVNIVKKTEVQTGGFSKESILPKRNSDKLIA | |
| | RKKDWDPKKYGGFDSPTVAYSVLVVAKVEKGKSKKLKSVKELLGITIMERSSFEKNPID | |
| | FLEAKGYKEVKKDLIIKLPKYSLFELENGRKRMLASAGELQKGNELALPSKYVNFLYLA | |
| | SHYEKLKGSPEDNEQKQLFVEQHKHYLDEIIEQISEFSKRVILADANLDKVLSAYNKHR | |
| | DKPIREQAENIIHLFTLTNLGAPAAFKYFDTTIDRKRYTSTKEVLDATLIHQSITGLYE | |
| | TRIDLSQLGGD | |
| Cas9 nickase | MDKKYSIGLDIGTNSVGWAVITDEYKVPSKKFKVLGNTDRHSIKKNLIGALLFDSGETA | 34 |
| Streptococcus | EATRLKRTARRRYTRRKNRICYLQEIFSNEMAKVDDSFFHRLEESFLVEEDKKHERHPI | |
| pyogenes | FGNIVDEVAYHEKYPTIYHLRKKLVDSTDKADLRLIYLALAHMIKFRGHFLIEGDLNPD | |
| Q992W2 Cas9 | NSDVDKLFIQLVQTYNQLFEENPINASGVDAKAILSARLSKSRRLENLIAQLPGEKKNG | |
| with E762A | LFGNLIALSLGLTPNFKSNFDLAEDAKLQLSKDTYDDDLDNLLAQIGDQYADLFLAAKN | |
| | LSDAILLSDILRVNTEITKAPLSASMIKRYDEHHQDLTLLKALVRQQLPEKYKEIFFDQ | |
| | SKNGYAGYIDGGASQEEFYKFIKPILEKMDGTEELLVKLNREDLLRKQRTFDNGSIPHQ | |
| | IHLGELHAILRRQEDFYPFLKDNREKIEKILTFRIPYYVGPLARGNSRFAWMTRKSEET | |
| | ITPWNFEEVVDKGASAQSFIERMTNFDKNLPNEKVLPKHSLLYEYFTVYNELTKVKYVT | |
| | EGMRKPAFLSGEQKKAIVDLLFKTNRKVTVKQLKEDYFKKIECFDSVEISGVEDRFNAS | |
| | LGTYHDLLKIIKDKDFLDNEENEDILEDIVLTLTLFEDREMIEERLKTYAHLFDDKVMK | |
| | QLKRRRYTGWGRLSRKLINGIRDKQSGKTILDFLKSDGFANRNFMQLIHDDSLTFKEDI | |
| | QKAQVSGQGDSLHEHIANLAGSPAIKKGILQTVKVVDELVKVMGRHKPENIVI [symbol] MAREN | |
| | QTTQKGQKNSRERMKRIEEGIKELGSQILKEHPVENTQLQNEKLYLYYLQNGRDMYVDQ | |
| | ELDINRLSDYDVDHIVPQSFLKDDSIDNKVLTRSDKNRGKSDNVPSEEVVKKMKNYWRQ | |
| | LLNAKLITQRKFDNLTKAERGGLSELDKAGFIKRQLVETRQITKHVAQILDSRMNTKYD | |
| | ENDKLIREVKVITLKSKLVSDFRKDFQFYKVREINNYHHAHDAYLNAVVGTALIKKYPK | |
| | LESEFVYGDYKVYDVRKMIAKSEQEIGKATAKYFFYSNIMNFFKTEITLANGEIRKRPL | |
| | IETNGETGEIVWDKGRDFATVRKVLSMPQVNIVKKTEVQTGGFSKESILPKRNSDKLIA | |
| | RKKDWDPKKYGGFDSPTVAYSVLVVAKVEKGKSKKLKSVKELLGITIMERSSFEKNPID | |
| | FLEAKGYKEVKKDLIIKLPKYSLFELENGRKRMLASAGELQKGNELALPSKYVNFLYLA | |
| | SHYEKLKGSPEDNEQKQLFVEQHKHYLDEIIEQISEFSKRVILADANLDKVLSAYNKHR | |
| | DKPIREQAENIIHLFTLTNLGAPAAFKYFDTTIDRKRYTSTKEVLDATLIHQSITGLYE | |
| | TRIDLSQLGGD | |
| Cas9 nickase | MDKKYSIGLDIGTNSVGWAVITDEYKVPSKKFKVLGNTDRHSIKKNLIGALLFDSGETA | 35 |
| Streptococcus | EATRLKRTARRRYTRRKNRICYLQEIFSNEMAKVDDSFFHRLEESFLVEEDKKHERHPI | |
| pyogenes | FGNIVDEVAYHEKYPTIYHLRKKLVDSTDKADLRLIYLALAHMIKFRGHFLIEGDLNPD | |
| Q99ZW2 Cas9 | NSDVDKLFIQLVQTYNQLFEENPINASGVDAKAILSARLSKSRRLENLIAQLPGEKKNG | |
| with H983A | LFGNLIALSLGLTPNFKSNFDLAEDAKLQLSKDTYDDDLDNLLAQIGDQYADLFLAAKN | |
| | LSDAILLSDILRVNTEITKAPLSASMIKRYDEHHQDLTLLKALVRQQLPEKYKEIFFDQ | |
| | SKNGYAGYIDGGASQEEFYKFIKPILEKMDGTEELLVKLNREDLLRKQRTFDNGSIPHQ | |
| | IHLGELHAILRRQEDFYPFLKDNREKIEKILTFRIPYYVGPLARGNSRFAWMTRKSEET | |
| | ITPWNFEEVVDKGASAQSFIERMTNFDKNLPNEKVLPKHSLLYEYFTVYNELTKVKYVT | |
| | EGMRKPAFLSGEQKKAIVDLLFKTNRKVTVKQLKEDYFKKIECFDSVEISGVEDRFNAS | |
| | LGTYHDLLKIIKDKDFLDNEENEDILEDIVLTLTLFEDREMIEERLKTYAHLFDDKVMK | |
| | QLKRRRYTGWGRLSRKLINGIRDKQSGKTILDFLKSDGFANRNFMQLIHDDSLTFKEDI | |
| | QKAQVSGQGDSLHEHIANLAGSPAIKKGILQTVKVVDELVKVMGRHKPENIVIEMAREN | |
| | QTTQKGQKNSRERMKRIEEGIKELGSQILKEHPVENTQLQNEKLYLYYLQNGRDMYVDQ | |
| | ELDINRLSDYDVDHIVPQSFLKDDSIDNKVLTRSDKNRGKSDNVPSEEVVKKMKNYWRQ | |
| | LLNAKLITQRKFDNLTKAERGGLSELDKAGFIKRQLVETRQITKHVAQILDSRMNTKYD | |
| | ENDKLIREVKVITLKSKLVSDFRKDFQFYKVREINNYH [symbol] AHDAYLNAVVGTALIKKYPK | |
| | LESEFVYGDYKVYDVRKMIAKSEQEIGKATAKYFFYSNTMNFFKTEITLANGEIRKRPL | |
| | IETNGETGEIVWDKGRDFATVRKVLSMPQVNIVKKTEVQTGGFSKESILPKRNSDKLIA | |
| | RKKDWDPKKYGGFDSPTVAYSVLVVAKVEKGKSKKLKSVKELLGITIMERSSFEKNPID | |
| | FLEAKGYKEVKKDLIIKLPKYSLFELENGRKRMLASAGELQKGNELALPSKYVNFLYLA | |
| | SHYEKLKGSPEDNEQKQLFVEQHKHYLDEIIEQISEFSKRVILADANLDKVLSAYNKHR | |
| | DKPIREQAENIIHLFTLTNLGAPAAFKYFDTTIDRKRYTSTKEVLDATLIHQSITGLYE | |
| | TRIDLSQLGGD | |
| Cas9 nickase | MDKKYSIGLDIGTNSVGWAVITDEYKVPSKKFKVLGNTDRHSIKKNLIGALLFDSGETA | 36 |
| Streptococcus | EATRLKRTARRRYTRRKNRICYLQEIFSNEMAKVDDSFFHRLEESFLVEEDKKHERHPI | |
| pyogenes | FGNIVDEVAYHEKYPTIYHLRKKLVDSTDKADLRLIYLALAHMIKFRGHFLIEGDLNPD | |
| Q99ZW2 Cas9 | NSDVDKLFIQLVQTYNQLFEENPINASGVDAKAILSARLSKSRRLENLIAQLPGEKKNG | |
| with D986A | LFGNLIALSLGLTPNFKSNFDLAEDAKLQLSKDTYDDDLDNLLAQIGDQYADLFLAAKN | |
| | LSDAILLSDILRVNTEITKAPLSASMIKRYDEHHQDLTLLKALVRQQLPEKYKEIFFDQ | |
| | SKNGYAGYIDGGASQEEFYKFIKPILEKMDGTEELLVKLNREDLLRKQRTFDNGSIPHQ | |
| | IHLGELHAILRRQEDFYPFLKDNREKIEKILTFRIPYYVGPLARGNSRFAWMTRKSEET | |
| | ITPWNFEEVVDKGASAQSFIERMTNFDKNLPNEKVLPKHSLLYEYFTVYNELTKVKYVT | |
| | EGMRKPAFLSGEQKKAIVDLLFKTNRKVTVKQLKEDYFKKIECFDSVEISGVEDRFNAS | |
| | LGTYHDLLKIIKDKDFLDNEENEDILEDIVLTLTLFEDREMIEERLKTYAHLFDDKVMK | |
| | QLKRRRYTGWGRLSRKLINGIRDKQSGKTILDFLKSDGFANRNFMQLIHDDSLTFKEDI | |
| | QKAQVSGQGDSLHEHIANLAGSPAIKKGILQTVKVVDELVKVMGRHKPENIVIEMAREN | |
| | QTTQKGQKNSRERMKRIEEGIKELGSQILKEHPVENTQLQNEKLYLYYLQNGRDMYVDQ | |
| | ELDINRLSDYDVDHIVPQSFLKDDSIDNKVLTRSDKNRGKSDNVPSEEVVKKMKNYWRQ | |
| | LLNAKLITQRKFDNLTKAERGGLSELDKAGFIKRQLVETRQITKHVAQILDSRMNTKYD | |
| | ENDKLIREVKVITLKSKLVSDFRKDFQFYKVREINNYHHAH [symbol] AYLNAVVGTALIKKYPK | |
| | LESEFVYGDYKVYDVRKMIAKSEQEIGKATAKYFFYSNIMNFFKTEITLANGEIRKRPL | |
| | IETNGETGEIVWDKGRDFATVRKVLSMPQVNIVKKTEVQTGGFSKESILPKRNSDKLIA | |
| | RKKDWDPKKYGGFDSPTVAYSVLVVAKVEKGKSKKLKSVKELLGITIMERSSFEKNPID | |
| | FLEAKGYKEVKKDLIIKLPKYSLFELENGRKRMLASAGELQKGNELALPSKYVNFLYLA | |
| | SHYEKLKGSPEDNEQKQLFVEQHKHYLDEIIEQISEFSKRVILADANLDKVLSAYNKHR | |
| | DKPIREQAENIIHLFTLTNLGAPAAFKYFDTTIDRKRYTSTKEVLDATLIHQSITGLYE | |
| | TRIDLSQLGGD | |

In another embodiment, the Cas9 nickase comprises a mutation in the HNH domain which inactivates the HNH nuclease activity. For example, mutations in histidine (H) 840 or asparagine (R) 863 have been reported as loss-of-function mutations of the HNH nuclease domain and the creation of a functional Cas9 nickase (e.g., Nishimasu et al., “Crystal structure of Cas9 in complex with guide RNA and target DNA,” Cell 156(5), 935-949, which is incorporated herein by reference). Thus, nickase mutations in the HNH domain could include H840X and R863X, wherein X is any amino acid other than the wild type amino acid. In certain embodiments, the nickase could be H840A or R863A or a combination thereof.

In various embodiments, the Cas9 nickase can have a mutation in the HNH nuclease domain and have one of the following amino acid sequences, or a variant thereof having an amino acid sequence that has at least 80%, at least 85%, at least 90%, at least 95%, or at least 99% sequence identity thereto.

| | | SEQ |
|---|---|---|
| | | ID |
| Description | Sequence | NO: |
| Cas9 nickase | MDKKYSIGLDIGTNSVGWAVITDEYKVPSKKFKVLGNTDRHSIKKNLIGALLFDSGETA | 37 |
| Streptococcus | EATRLKRTARRRYTRRKNRICYLQEIFSNEMAKVDDSFFHRLEESFLVEEDKKHERHPI | |
| pyogenes | FGNIVDEVAYHEKYPTIYHLRKKLVDSTDKADLRLIYLALAHMIKFRGHFLIEGDLNPD | |
| Q992W2 Cas9 | NSDVDKLFIQLVQTYNQLFEENPINASGVDAKAILSARLSKSRRLENLIAQLPGEKKNG | |
| with H840 [symbol] , | LFGNLIALSLGLTPNFKSNFDLAEDAKLQLSKDTYDDDLDNLLAQIGDQYADLFLAAKN | |
| wherein X is | LSDAILLSDILRVNTEITKAPLSASMIKRYDEHHQDLTLLKALVRQQLPEKYKEIFFDQ | |
| any | SKNGYAGYIDGGASQEEFYKFIKPILEKMDGTEELLVKLNREDLLRKQRTFDNGSIPHQ | |
| alternate | IHLGELHAILRRQEDFYPFLKDNREKIEKILTFRIPYYVGPLARGNSRFAWMTRKSEET | |
| amino acid | ITPWNFEEVVDKGASAQSFIERMTNFDKNLPNEKVLPKHSLLYEYFTVYNELTKVKYVT | |
| | EGMRKPAFLSGEQKKAIVDLLFKTNRKVTVKQLKEDYFKKIECFDSVEISGVEDRFNAS | |
| | LGTYHDLLKIIKDKDFLDNEENEDILEDIVLTLTLFEDREMIEERLKTYAHLFDDKVMK | |
| | QLKRRRYTGWGRLSRKLINGIRDKQSGKTILDFLKSDGFANRNFMQLIHDDSLTFKEDI | |
| | QKAQVSGQGDSLHEHIANLAGSPAIKKGILQTVKVVDELVKVMGRHKPENIVIEMAREN | |
| | QTTQKGQKNSRERMKRIEEGIKELGSQILKEHPVENTQLQNEKLYLYYLQNGRDMYVDQ | |
| | ELDINRLSDYDVD [symbol] IVPQSFLKDDSIDNKVLTRSDKNRGKSDNVPSEEVVKKMKNYWRQ | |
| | LLNAKLITQRKFDNLTKAERGGLSELDKAGFIKRQLVETRQITKHVAQILDSRMNTKYD | |
| | ENDKLIREVKVITLKSKLVSDFRKDFQFYKVREINNYHHAHDAYLNAVVGTALIKKYPK | |
| | LESEFVYGDYKVYDVRKMIAKSEQEIGKATAKYFFYSNIMNFFKTEITLANGEIRKRPL | |
| | IETNGETGEIVWDKGRDFATVRKVLSMPQVNIVKKTEVQTGGFSKESILPKRNSDKLIA | |
| | RKKDWDPKKYGGFDSPTVAYSVLVVAKVEKGKSKKLKSVKELLGITIMERSSFEKNPID | |
| | FLEAKGYKEVKKDLIIKLPKYSLFELENGRKRMLASAGELQKGNELALPSKYVNFLYLA | |
| | SHYEKLKGSPEDNEQKQLFVEQHKHYLDEIIEQISEFSKRVILADANLDKVLSAYNKHR | |
| | DKPIREQAENIIHLFTLTNLGAPAAFKYFDTTIDRKRYTSTKEVLDATLIHQSITGLYE | |
| | TRIDLSQLGGD | |
| Cas9 nickase | MDKKYSIGLDIGTNSVGWAVITDEYKVPSKKFKVLGNTDRHSIKKNLIGALLFDSGETA | 38 |
| Streptococcus | EATRLKRTARRRYTRRKNRICYLQEIFSNEMAKVDDSFFHRLEESFLVEEDKKHERHPI | |
| pyogenes | FGNIVDEVAYHEKYPTIYHLRKKLVDSTDKADLRLIYLALAHMIKFRGHFLIEGDLNPD | |
| Q99ZW2 Cas9 | NSDVDKLFIQLVQTYNQLFEENPINASGVDAKAILSARLSKSRRLENLIAQLPGEKKNG | |
| with H840 [symbol] , | LFGNLIALSLGLTPNFKSNFDLAEDAKLQLSKDTYDDDLDNLLAQIGDQYADLFLAAKN | |
| wherein X is | LSDAILLSDILRVNTEITKAPLSASMIKRYDEHHQDLTLLKALVRQQLPEKYKEIFFDQ | |
| any | SKNGYAGYIDGGASQEEFYKFIKPILEKMDGTEELLVKLNREDLLRKQRTFDNGSIPHQ | |
| alternate | IHLGELHAILRRQEDFYPFLKDNREKIEKILTFRIPYYVGPLARGNSRFAWMTRKSEET | |
| amino acid | ITPWNFEEVVDKGASAQSFIERMTNFDKNLPNEKVLPKHSLLYEYFTVYNELTKVKYVT | |
| | EGMRKPAFLSGEQKKAIVDLLFKTNRKVTVKQLKEDYFKKIECFDSVEISGVEDRFNAS | |
| | LGTYHDLLKIIKDKDFLDNEENEDILEDIVLTLTLFEDREMIEERLKTYAHLFDDKVMK | |
| | QLKRRRYTGWGRLSRKLINGIRDKQSGKTILDFLKSDGFANRNFMQLIHDDSLTFKEDI | |
| | QKAQVSGQGDSLHEHIANLAGSPAIKKGILQTVKVVDELVKVMGRHKPENIVIEMAREN | |
| | QTTQKGQKNSRERMKRIEEGIKELGSQILKEHPVENTQLQNEKLYLYYLQNGRDMYVDQ | |
| | ELDINRLSDYDVD [symbol] IVPQSFLKDDSIDNKVLTRSDKNRGKSDNVPSEEVVKKMKNYWRQ | |
| | LLNAKLITQRKFDWLTKAERGGLSELDKAGFIKRQLVETRQITKHVAQILDSRMNTKYD | |
| | ENDKLIREVKVITLKSKLVSDFRKDFQFYKVREINNYHHAHDAYLNAVVGTALIKKYPK | |
| | LESEFVYGDYKVYDVRKMIAKSEQEIGKATAKYFFYSNIMNFFKTEITLANGEIRKRPL | |
| | IETNGETGEIVWDKGRDFATVRKVLSMPQVNIVKKTEVQTGGFSKESILPKRNSDKLIA | |
| | RKKDWDPKKYGGFDSPTVAYSVLVVAKVEKGKSKKLKSVKELLGITIMERSSFEKNPID | |
| | FLEAKGYKEVKKDLIIKLPKYSLFELENGRKRMLASAGELQKGNELALPSKYVNFLYLA | |
| | SHYEKLKGSPEDNEQKQLFVEQHKHYLDEIIEQISEFSKRVILADANLDKVLSAYNKHR | |
| | DKPIREQAENIIHLFTLTNLGAPAAFKYFDTTIDRKRYTSTKEVLDATLIHQSITGLYE | |
| | TRIDLSQLGGD | |
| Cas9 nickase | MDKKYSIGLDIGTNSVGWAVITDEYKVPSKKFKVLGNTDRHSIKKNLIGALLFDSGETA | 39 |
| Streptococcus | EATRLKRTARRRYTRRKNRICYLQEIFSNEMAKVDDSFFHRLEESFLVEEDKKHERHPI | |
| pyogenes | FGNIVDEVAYHEKYPTIYHLRKKLVDSTDKADLRLIYLALAHMIKFRGHFLIEGDLNPD | |
| Q99ZW2 Cas9 | NSDVDKLFIQLVQTYNQLFEENPINASGVDAKAILSARLSKSRRLENLIAQLPGEKKNG | |
| with R863X, | LFGNLIALSLGLTPNFKSNFDLAEDAKLQLSKDTYDDDLDNLLAQIGDQYADLFLAAKN | |
| wherein X is | LSDAILLSDILRVNTEITKAPLSASMIKRYDEHHQDLTLLKALVRQQLPEKYKEIFFDQ | |
| any | SKNGYAGYIDGGASQEEFYKFIKPILEKMDGTEELLVKLNREDLLRKQRTFDNGSIPHQ | |
| alternate | IHLGELHAILRRQEDFYPFLKDNREKIEKILTFRIPYYVGPLARGNSRFAWMTRKSEET | |
| amino acid | ITPWNFEEVVDKGASAQSFIERMTNFDKNLPNEKVLPKHSLLYEYFTVYNELTKVKYVT | |
| | EGMRKPAFLSGEQKKAIVDLLFKTNRKVTVKQLKEDYFKKIECFDSVEISGVEDRFNAS | |
| | LGTYHDLLKIIKDKDFLDNEENEDILEDIVLTLTLFEDREMIEERLKTYAHLFDDKVMK | |
| | QLKRRRYTGWGRLSRKLINGIRDKQSGKTILDFLKSDGFANRNFMQLIHDDSLTFKEDI | |
| | QKAQVSGQGDSLHEHIANLAGSPAIKKGILQTVKVVDELVKVMGRHKPENIVIEMAREN | |
| | QTTQKGQKNSRERMKRIEEGIKELGSQILKEHPVENTQLQNEKLYLYYLQNGRDMYVDQ | |
| | ELDINRLSDYDVDHIVPQSFLKDDSIDNKVLTRSDKN [symbol] GKSDNVPSEEVVKKMKNYWRQ | |
| | LLNAKLITQRKFDNLTKAERGGLSELDKAGFIKRQLVETRQITKHVAQILDSRMNTKYD | |
| | ENDKLIREVKVITLKSKLVSDFRKDFQFYKVREINNYHHAHDAYLNAVVGTALIKKYPK | |
| | LESEFVYGDYKVYDVRKMIAKSEQEIGKATAKYFFYSNIMNFFKTEITLANGEIRKRPL | |
| | IETNGETGEIVWDKGRDFATVRKVLSMPQVNIVKKTEVQTGGFSKESILPKRNSDKLIA | |
| | RKKDWDPKKYGGFDSPTVAYSVLVVAKVEKGKSKKLKSVKELLGITIMERSSFEKNPID | |
| | FLEAKGYKEVKKDLIIKLPKYSLFELENGRKRMLASAGELQKGNELALPSKYVNFLYLA | |
| | SHYEKLKGSPEDNEQKQLFVEQHKHYLDEIIEQISEFSKRVILADANLDKVLSAYNKHR | |
| | DKPIREQAENIIHLFTLTNLGAPAAFKYFDTTIDRKRYTSTKEVLDATLIHQSITGLYE | |
| | TRIDLSQLGGD | |
| Cas9 nickase | MDKKYSIGLDIGTNSVGWAVITDEYKVPSKKFKVLGNTDRHSIKKNLIGALLFDSGETA | 40 |
| Streptococcus | EATRLKRTARRRYTRRKNRICYLQEIFSNEMAKVDDSFFHRLEESFLVEEDKKHERHPI | |
| pyogenes | FGNIVDEVAYHEKYPTIYHLRKKLVDSTDKADLRLIYLALAHMIKFRGHFLIEGDLNPD | |
| Q99ZW2 Cas9 | NSDVDKLFIQLVQTYNQLFEENPINASGVDAKAILSARLSKSRRLENLIAQLPGEKKNG | |
| with R863 [symbol] , | LFGNLIALSLGLTPNFKSNFDLAEDAKLQLSKDTYDDDLDNLLAQIGDQYADLFLAAKN | |
| wherein X is | LSDAILLSDILRVNTEITKAPLSASMIKRYDEHHQDLTLLKALVRQQLPEKYKEIFFDQ | |
| any | SKNGYAGYIDGGASQEEFYKFIKPILEKMDGTEELLVKLNREDLLRKQRTFDNGSIPHQ | |
| alternate | IHLGELHAILRRQEDFYPFLKDNREKIEKILTFRIPYYVGPLARGNSRFAWMTRKSEET | |
| amino acid | ITPWNFEEVVDKGASAQSFIERMTNFDKNLPNEKVLPKHSLLYEYFTVYNELTKVKYVT | |
| | EGMRKPAFLSGEQKKAIVDLLFKTNRKVTVKQLKEDYFKKIECFDSVEISGVEDRFNAS | |
| | LGTYHDLLKIIKDKDFLDNEENEDILEDIVLTLTLFEDREMIEERLKTYAHLFDDKVMK | |
| | QLKRRRYTGWGRLSRKLINGIRDKQSGKTILDFLKSDGFANRNFMQLIHDDSLTFKEDI | |
| | QKAQVSGQGDSLHEHIANLAGSPAIKKGILQTVKVVDELVKVMGRHKPENIVIEMAREN | |
| | QTTQKGQKNSRERMKRIEEGIKELGSQILKEHPVENTQLQNEKLYLYYLQNGRDMYVDQ | |
| | ELDINRLSDYDVDHIVPQSFLKDDSIDNKVLTRSDKN [symbol] GKSDNVPSEEVVKKMKNYWRQ | |
| | LLNAKLITQRKFDNLTKAERGGLSELDKAGFIKRQLVETRQITKHVAQILDSRMNTKYD | |
| | ENDKLIREVKVITLKSKLVSDFRKDFQFYKVREINNYHHAHDAYLNAVVGTALIKKYPK | |
| | LESEFVYGDYKVYDVRKMIAKSEQEIGKATAKYFFYSNIMNFFKTEITLANGEIRKRPL | |
| | IETNGETGEIVWDKGRDFATVRKVLSMPQVNIVKKTEVQTGGFSKESILPKRNSDKLIA | |
| | RKKDWDPKKYGGFDSPTVAYSVLVVAKVEKGKSKKLKSVKELLGITIMERSSFEKNPID | |
| | FLEAKGYKEVKKDLIIKLPKYSLFELENGRKRMLASAGELQKGNELALPSKYVNFLYLA | |
| | SHYEKLKGSPEDNEQKQLFVEQHKHYLDEIIEQISEFSKRVILADANLDKVLSAYNKHR | |
| | DKPIREQAENIIHLFTLTNLGAPAAFKYFDTTIDRKRYTSTKEVLDATLIHQSITGLYE | |
| | TRIDLSQLGGD | |

In some embodiments, the N-terminal methionine is removed from a Cas9 nickase, or from any Cas9 variant, ortholog, or equivalent disclosed or contemplated herein. For example, methionine-minus Cas9 nickases include the following sequences, or a variant thereof having an amino acid sequence that has at least 80%, at least 85%, at least 90%, at least 95%, or at least 99% sequence identity thereto.

| Description | Sequence |
|---|---|
| Cas9 nickase | DKKYSIGLDIGTNSVGWAVITDEYKVPSKKFKVLGNTDRHSIKKNLIGALLFDSGETAEATRLKRTARRR |
| (Met minus) | YTRRKNRICYLQEIFSNEMAKVDDSFFHRLEESFLVEEDKKHERHPIFGNIVDEVAYHEKYPTIYHLRKK |
| Streptococcus | LVDSTDKADLRLIYLALAHMIKFRGHFLIEGDLNPDNSDVDKLFIQLVQTYNQLFEENPINASGVDAKAI |
| pyogenes | LSARLSKSRRLENLIAQLPGEKKNGLFGNLIALSLGLTPNFKSNFDLAEDAKLQLSKDTYDDDLDNLLAQ |
| Q992W2 Cas9 | IGDQYADLFLAAKNLSDAILLSDILRVNTEITKAPLSASMIKRYDEHHQDLTLLKALVRQQLPEKYKEIE |
| with H840 [symbol] , | FDQSKNGYAGYIDGGASQEEFYKFIKPILEKMDGTEELLVKLNREDLLRKQRTFDNGSIPHQIHLGELHA |
| wherein X is | ILRRQEDFYPFLKDNREKIEKILTFRIPYYVGPLARGNSRFAWMTRKSEETITPWNFEEVVDKGASAQSF |
| any | IERMTNFDKNLPNEKVLPKHSLLYEYFTVYNELTKVKYVTEGMRKPAFLSGEQKKAIVDLLFKTNRKVTV |
| alternate | KQLKEDYFKKIECFDSVEISGVEDRFNASLGTYHDLLKIIKDKDFLDNEENEDILEDIVLTLTLFEDREM |
| amino acid | IEERLKTYAHLFDDKVMKQLKRRRYTGWGRLSRKLINGIRDKQSGKTILDFLKSDGFANRNFMQLIHDDS |
| | LTFKEDIQKAQVSGQGDSLHEHIANLAGSPAIKKGILQTVKVVDELVKVMGRHKPENIVIEMARENQTTQ |
| | KGQKNSRERMKRIEEGIKELGSQILKEHPVENTQLQNEKLYLYYLQNGRDMYVDQELDINRLSDYDVD [symbol] I |
| | VPQSFLKDDSIDNKVLTRSDKNRGKSDNVPSEEVVKKMKNYWRQLLNAKLITQRKFDNLTKAERGGLSEL |
| | DKAGFIKRQLVETRQITKHVAQILDSRMNTKYDENDKLIREVKVITLKSKLVSDFRKDFQFYKVREINNY |
| | HHAHDAYLNAVVGTALIKKYPKLESEFVYGDYKVYDVRKMIAKSEQEIGKATAKYFFYSNIMNFFKTEIT |
| | LANGEIRKRPLIETNGETGEIVWDKGRDFATVRKVLSMPQVNIVKKTEVQTGGFSKESILPKRNSDKLIA |
| | RKKDWDPKKYGGFDSPTVAYSVLVVAKVEKGKSKKLKSVKELLGITIMERSSFEKNPIDFLEAKGYKEVK |
| | KDLIIKLPKYSLFELENGRKRMLASAGELQKGNELALPSKYVNFLYLASHYEKLKGSPEDNEQKQLFVEQ |
| | HKHYLDEIIEQISEFSKRVILADANLDKVLSAYNKHRDKPIREQAENIIHLFTLTNLGAPAAFKYFDTTI |
| | DRKRYTSTKEVLDATLIHQSITGLYETRIDLSQLGGD (SEQ ID NO: 41) |
| Cas9 nickase | DKKYSIGLDIGTNSVGWAVITDEYKVPSKKFKVLGNTDRHSIKKNLIGALLFDSGETAEATRLKRTARRR |
| (Met minus) | YTRRKNRICYLQEIFSNEMAKVDDSFFHRLEESFLVEEDKKHERHPIFGNIVDEVAYHEKYPTIYHLRKK |
| Streptococcus | LVDSTDKADLRLIYLALAHMIKFRGHFLIEGDLNPDNSDVDKLFIQLVQTYNQLFEENPINASGVDAKAI |
| pyogenes | LSARLSKSRRLENLIAQLPGEKKNGLFGNLIALSLGLTPNFKSNFDLAEDAKLQLSKDTYDDDLDNLLAQ |
| Q992W2 Cas9 | IGDQYADLFLAAKNLSDAILLSDILRVNTEITKAPLSASMIKRYDEHHQDLTLLKALVRQQLPEKYKEIF |
| with H840 [symbol] , | FDQSKNGYAGYIDGGASQEEFYKFIKPILEKMDGTEELLVKLNREDLLRKQRTFDNGSIPHQIHLGELHA |
| wherein X is | ILRRQEDFYPFLKDNREKIEKILTFRIPYYVGPLARGNSRFAWMTRKSEETITPWNFEEVVDKGASAQSE |
| | IERMTNFDKNLPNEKVLPKHSLLYEYFTVYNELTKVKYVTEGMRKPAFLSGEQKKAIVDLLFKTNRKVTV |
| any | KQLKEDYFKKIECFDSVEISGVEDRFNASLGTYHDLLKIIKDKDFLDNEENEDILEDIVLTLTLFEDREM |
| alternate | IEERLKTYAHLFDDKVMKQLKRRRYTGWGRLSRKLINGIRDKQSGKTILDFLKSDGFANRNFMQLIHDDS |
| amino acid | LTFKEDIQKAQVSGQGDSLHEHIANLAGSPAIKKGILQTVKVVDELVKVMGRHKPENIVIEMARENQTTQ |
| | KGQKNSRERMKRIEEGIKELGSQILKEHPVENTQLQNEKLYLYYLQNGRDMYVDQELDINRLSDYDVD [symbol] I |
| | VPQSFLKDDSIDNKVLTRSDKNRGKSDNVPSEEVVKKMKNYWRQLLNAKLITQRKFDNLTKAERGGLSEL |
| | DKAGFIKRQLVETRQITKHVAQTLDSRMNTKYDENDKLIREVKVITLKSKLVSDFRKDFQFYKVREINNY |
| | HHAHDAYLNAVVGTALIKKYPKLESEFVYGDYKVYDVRKMIAKSEQEIGKATAKYFFYSNIMNFFKTEIT |
| | LANGEIRKRPLIETNGETGEIVWDKGRDFATVRKVLSMPQVNIVKKTEVQTGGFSKESILPKRNSDKLIA |
| | RKKDWDPKKYGGFDSPTVAYSVLVVAKVEKGKSKKLKSVKELLGITIMERSSFEKNPIDFLEAKGYKEVK |
| | KDLIIKLPKYSLFELENGRKRMLASAGELQKGNELALPSKYVNFLYLASHYEKLKGSPEDNEQKQLFVEQ |
| | HKHYLDEIIEQISEFSKRVILADANLDKVLSAYNKHRDKPIREQAENIIHLFTLTNLGAPAAFKYFDTTI |
| | DRKRYTSTKEVLDATLIHQSITGLYETRIDLSQLGGD (SEQ ID NO: 42) |
| Cas9 nickase | DKKYSIGLDIGTNSVGWAVITDEYKVPSKKFKVLGNTDRHSIKKNLIGALLFDSGETAEATRLKRTARRR |
| (Met minus) | YTRRKNRICYLQEIFSNEMAKVDDSFFHRLEESFLVEEDKKHERHPIFGNIVDEVAYHEKYPTIYHLRKK |
| Streptococcus | LVDSTDKADLRLIYLALAHMIKFRGHFLIEGDLNPDNSDVDKLFIQLVQTYNQLFEENPINASGVDAKAI |
| pyogenes | LSARLSKSRRLENLIAQLPGEKKNGLFGNLIALSLGLTPNFKSNFDLAEDAKLQLSKDTYDDDLDNLLAQ |
| Q992W2 Cas9 | IGDQYADLFLAAKNLSDAILLSDILRVNTEITKAPLSASMIKRYDEHHQDLTLLKALVRQQLPEKYKEIF |
| with R863X, | FDQSKNGYAGYIDGGASQEEFYKFIKPILEKMDGTEELLVKLNREDLLRKQRTFDNGSIPHQIHLGELHA |
| wherein X is | ILRRQEDFYPFLKDNREKIEKILTFRIPYYVGPLARGNSRFAWMTRKSEETITPWNFEEVVDKGASAQSF |
| any | IERMTNFDKNLPNEKVLPKHSLLYEYFTVYNELTKVKYVTEGMRKPAFLSGEQKKAIVDLLFKTNRKVTV |
| alternate | KQLKEDYFKKIECFDSVEISGVEDRFNASLGTYHDLLKIIKDKDFLDNEENEDILEDIVLTLTLFEDREM |
| amino acid | IEERLKTYAHLFDDKVMKQLKRRRYTGWGRLSRKLINGIRDKQSGKTILDFLKSDGFANRNFMQLIHDDS |
| | LTFKEDIQKAQVSGQGDSLHEHIANLAGSPAIKKGILQTVKVVDELVKVMGRHKPENIVIEMARENQTTQ |
| | KGQKNSRERMKRIEEGIKELGSQILKEHPVENTQLQNEKLYLYYLQNGRDMYVDQELDINRLSDYDVDHI |
| | VPQSFLKDDSIDNKVLTRSDKN [symbol] GKSDNVPSEEVVKKMKNYWRQLLNAKLITQRKFDNLTKAERGGLSEL |
| | DKAGFIKRQLVETRQITKHVAQTLDSRMNTKYDENDKLIREVKVITLKSKLVSDFRKDFQFYKVREINNY |
| | HHAHDAYLNAVVGTALIKKYPKLESEFVYGDYKVYDVRKMIAKSEQEIGKATAKYFFYSNIMNFFKTEIT |
| | LANGEIRKRPLIETNGETGEIVWDKGRDFATVRKVLSMPQVNIVKKTEVQTGGFSKESILPKRNSDKLIA |
| | RKKDWDPKKYGGFDSPTVAYSVLVVAKVEKGKSKKLKSVKELLGITIMERSSFEKNPIDFLEAKGYKEVK |
| | KDLIIKLPKYSLFELENGRKRMLASAGELQKGNELALPSKYVNFLYLASHYEKLKGSPEDNEQKQLFVEQ |
| | HKHYLDEIIEQISEFSKRVILADANLDKVLSAYNKHRDKPIREQAENIIHLFTLTNLGAPAAFKYFDTTI |
| | DRKRYTSTKEVLDATLIHQSITGLYETRIDLSQLGGD(SEQ ID NO: 43) |
| Cas9 nickase | DKKYSIGLDIGTNSVGWAVITDEYKVPSKKFKVLGNTDRHSIKKNLIGALLFDSGETAEATRLKRTARRR |
| (Met minus) | YTRRKNRICYLQEIFSNEMAKVDDSFFHRLEESFLVEEDKKHERHPIFGNIVDEVAYHEKYPTIYHLRKK |
| Streptococcus | LVDSTDKADLRLIYLALAHMIKFRGHFLIEGDLNPDNSDVDKLFIQLVQTYNQLFEENPINASGVDAKAI |
| pyogenes | LSARLSKSRRLENLIAQLPGEKKNGLFGNLIALSLGLTPNFKSNFDLAEDAKLQLSKDTYDDDLDNLLAQ |
| Q992W2 Cas9 | IGDQYADLFLAAKNLSDAILLSDILRVNTEITKAPLSASMIKRYDEHHQDLTLLKALVRQQLPEKYKEIF |
| with R863 [symbol] , | FDQSKNGYAGYIDGGASQEEFYKFIKPILEKMDGTEELLVKLNREDLLRKQRTFDNGSIPHQIHLGELHA |
| wherein X is | ILRRQEDFYPFLKDNREKIEKILTFRIPYYVGPLARGNSRFAWMTRKSEETITPWNFEEVVDKGASAQSE |
| any | IERMTNFDKNLPNEKVLPKHSLLYEYFTVYNELTKVKYVTEGMRKPAFLSGEQKKAIVDLLFKTNRKVTV |
| alternate | KQLKEDYFKKIECFDSVEISGVEDRFNASLGTYHDLLKIIKDKDFLDNEENEDILEDIVLTLTLFEDREM |
| amino acid | IEERLKTYAHLFDDKVMKQLKRRRYTGWGRLSRKLINGIRDKQSGKTILDFLKSDGFANRNFMQLIHDDS |
| | LTFKEDIQKAQVSGQGDSLHEHIANLAGSPAIKKGILQTVKVVDELVKVMGRHKPENIVIEMARENQTTQ |
| | KGQKNSRERMKRIEEGIKELGSQILKEHPVENTQLQNEKLYLYYLQNGRDMYVDQELDINRLSDYDVDHI |
| | VPQSFLKDDSIDNKVLTRSDKN [symbol] GKSDNVPSEEVVKKMKNYWRQLLNAKLITQRKFDNLTKAERGGLSEL |
| | DKAGFIKRQLVETRQITKHVAQTLDSRMNTKYDENDKLIREVKVITLKSKLVSDFRKDFQFYKVREINNY |
| | HHAHDAYLNAVVGTALIKKYPKLESEFVYGDYKVYDVRKMIAKSEQEIGKATAKYFFYSNIMNFFKTEIT |
| | LANGEIRKRPLIETNGETGEIVWDKGRDFATVRKVLSMPQVNIVKKTEVQTGGFSKESILPKRNSDKLIA |
| | RKKDWDPKKYGGFDSPTVAYSVLVVAKVEKGKSKKLKSVKELLGITIMERSSFEKNPIDFLEAKGYKEVK |
| | KDLIIKLPKYSLFELENGRKRMLASAGELQKGNELALPSKYVNFLYLASHYEKLKGSPEDNEQKQLFVEQ |
| | HKHYLDEIIEQISEFSKRVILADANLDKVLSAYNKHRDKPIREQAENIIHLFTLTNLGAPAAFKYFDTTI |
| | DRKRYTSTKEVLDATLIHQSITGLYETRIDLSQLGGD (SEQ ID NO: 44) |

(5) Other Cas9 Variants

Besides dead Cas9 and Cas9 nickase variants, the Cas9 proteins used herein may also include other “Cas9 variants” having at least about 70% identical, at least about 80% identical, at least about 90% identical, at least about 95% identical, at least about 96% identical, at least about 97% identical, at least about 98% identical, at least about 99% identical, at least about 99.5% identical, or at least about 99.9% identical to any reference Cas9 protein, including any wild type Cas9, or mutant Cas9 (e.g., a dead Cas9 or Cas9 nickase), or fragment Cas9, or circular permutant Cas9, or other variant of Cas9 disclosed herein or known in the art. In some embodiments, a Cas9 variant may have 1, 2, 3, 4, 5, 6, 7, 8, 9, 10, 11, 12, 13, 14, 15, 16, 17, 18, 19, 20, 21, 22, 21, 24, 25, 26, 27, 28, 29, 30, 31, 32, 33, 34, 35, 36, 37, 38, 39, 40, 41, 42, 43, 44, 45, 46, 47, 48, 49, 50 or more amino acid changes compared to a reference Cas9. In some embodiments, the Cas9 variant comprises a fragment of a reference Cas9 (e.g., a gRNA binding domain or a DNA-cleavage domain), such that the fragment is at least about 70% identical, at least about 80% identical, at least about 90% identical, at least about 95% identical, at least about 96% identical, at least about 97% identical, at least about 98% identical, at least about 99% identical, at least about 99.5% identical, or at least about 99.9% identical to the corresponding fragment of wild type Cas9. In some embodiments, the fragment is at least 30%, at least 35%, at least 40%, at least 45%, at least 50%, at least 55%, at least 60%, at least 65%, at least 70%, at least 75%, at least 80%, at least 85%, at least 90%, at least 95% identical, at least 96%, at least 97%, at least 98%, at least 99%, or at least 99.5% of the amino acid length of a corresponding wild type Cas9 (e.g., SEQ ID NO: 5).

In some embodiments, the disclosure also may utilize Cas9 fragments which retain their functionality and which are fragments of any herein disclosed Cas9 protein. In some embodiments, the Cas9 fragment is at least 100 amino acids in length. In some embodiments, the fragment is at least 100, 150, 200, 250, 300, 350, 400, 450, 500, 550, 600, 650, 700, 750, 800, 850, 900, 950, 1000, 1050, 1100, 1150, 1200, 1250, or at least 1300 amino acids in length.

In various embodiments, the base editors disclosed herein may comprise one of the Cas9 variants described as follows, or a Cas9 variant thereof having at least about 70% identical, at least about 80% identical, at least about 90% identical, at least about 95% identical, at least about 96% identical, at least about 97% identical, at least about 98% identical, at least about 99% identical, at least about 99.5% identical, or at least about 99.9% identical to any reference Cas9 variants.

(6) Small-Sized Cas9 Variants

In some embodiments, the base editors contemplated herein can include a Cas9 protein that is of smaller molecular weight than the canonical SpCas9 sequence. In some embodiments, the smaller-sized Cas9 variants may facilitate delivery to cells, e.g., by an expression vector, nanoparticle, or other means of delivery.

The canonical SpCas9 protein is 1368 amino acids in length and has a predicted molecular weight of 158 kilodaltons. The term “small-sized Cas9 variant”, as used herein, refers to any Cas9 variant-naturally occurring, engineered, or otherwise—that is less than at least 1300 amino acids, or at least less than 1290 amino acids, or than less than 1280 amino acids, or less than 1270 amino acid, or less than 1260 amino acid, or less than 1250 amino acids, or less than 1240 amino acids, or less than 1230 amino acids, or less than 1220 amino acids, or less than 1210 amino acids, or less than 1200 amino acids, or less than 1190 amino acids, or less than 1180 amino acids, or less than 1170 amino acids, or less than 1160 amino acids, or less than 1150 amino acids, or less than 1140 amino acids, or less than 1130 amino acids, or less than 1120 amino acids, or less than 1110 amino acids, or less than 1100 amino acids, or less than 1050 amino acids, or less than 1000 amino acids, or less than 950 amino acids, or less than 900 amino acids, or less than 850 amino acids, or less than 800 amino acids, or less than 750 amino acids, or less than 700 amino acids, or less than 650 amino acids, or less than 600 amino acids, or less than 550 amino acids, or less than 500 amino acids, but at least larger than about 400 amino acids and retaining the required functions of the Cas9 protein.

In various embodiments, the base editors disclosed herein may comprise one of the small-sized Cas9 variants described as follows, or a Cas9 variant thereof having at least about 70% identical, at least about 80% identical, at least about 90% identical, at least about 95% identical, at least about 96% identical, at least about 97% identical, at least about 98% identical, at least about 99% identical, at least about 99.5% identical, or at least about 99.9% identical to any reference small-sized Cas9 protein.

| | | SEQ |
|---|---|---|
| | | ID |
| Description | Sequence | NO: |
| SaCas 9 | MGKRNYILGLDIGITSVGYGIIDYETRDVIDAGVRLFKEANVENNEGRRSKRGARRLKRR | 45 |
| Staphylococcus | RRHRIQRVKKLLFDYNLLTDHSELSGINPYEARVKGLSQKLSEEEFSAALLHLAKRRGVH | |
| aureus | NVNEVEEDTGNELSTKEQISRNSKALEEKYVAELQLERLKKDGEVRGSINRFKTSDYVKE | |
| 1053 AA | AKQLLKVQKAYHQLDQSFIDTYIDLLETRRTYYEGPGEGSPFGWKDIKEWYEMLMGHCTY | |
| 123 kDa | FPEELRSVKYAYNADLYNALNDLNNLVITRDENEKLEYYEKFQIIENVFKQKKKPTLKQI | |
| | AKEILVNEEDIKGYRVTSTGKPEFTNLKVYHDIKDITARKEIIENAELLDQIAKILTIYQ | |
| | SSEDIQEELTNLNSELTQEEIEQISNLKGYTGTHNLSLKAINLILDELWHTNDNQIAIFN | |
| | RLKLVPKKVDLSQQKEIPTTLVDDFILSPVVKRSFIQSIKVINAIIKKYGLPNDIIIELA | |
| | REKNSKDAQKMINEMQKRNRQTNERIEEIIRTTGKENAKYLIEKIKLHDMQEGKCLYSLE | |
| | AIPLEDLLNNPFNYEVDHIIPRSVSFDNSFNNKVLVKQEENSKKGNRTPFQYLSSSDSKI | |
| | SYETFKKHILNLAKGKGRISKTKKEYLLEERDINRFSVQKDFINRNLVDTRYATRGLMNL | |
| | LRSYFRVNNLDVKVKSINGGFTSFLRRKWKFKKERNKGYKHHAEDALIIANADFIFKEWK | |
| | KLDKAKKVMENQMFEEKQAESMPEIETEQEYKEIFITPHQIKHIKDFKDYKYSHRVDKKP | |
| | NRKLINDTLYSTRKDDKGNTLIVNNLNGLYDKDNDKLKKLINKSPEKLLMYHHDPQTYQK | |
| | LKLIMEQYGDEKNPLYKYYEETGNYLTKYSKKDNGPVIKKIKYYGNKLNAHLDITDDYPN | |
| | SRNKVVKLSLKPYRFDVYLDNGVYKFVTVKNLDVIKKENYYEVNSKCYEEAKKLKKISNQ | |
| | AEFIASFYKNDLIKINGELYRVIGVNNDLLNRIEVNMIDITYREYLENMNDKRPPHIIKT | |
| | IASKTQSIKKYSTDILGNLYEVKSKKHPQIIKK | |
| NmeCas 9 | MAAFKPNSINYILGLDIGIASVGWAMVEIDEEENPIRLIDLGVRVFERAEVPKTGDSLAM | 4b |
| N. | ARRLARSVRRLTRRRAHRLLRTRRLLKREGVLQAANFDENGLIKSLPNTPWQLRAAALDR | |
| meningitidis | KLTPLEWSAVLLHLIKHRGYLSQRKNEGETADKELGALLKGVAGNAHALQTGDFRTPAEL | |
| 1083 AA | ALNKFEKESGHIRNQRSDYSHTFSRKDLQAELILLFEKQKEFGNPHVSGGLKEGIETLLM | |
| 124.5 kDa | TQRPALSGDAVQKMLGHCTFEPAEPKAAKNTYTAERFIWLTKLNNLRILEQGSERPLTDT | |
| | ERATLMDEPYRKSKLTYAQARKLLGLEDTAFFKGLRYGKDNAEASTLMEMKAYHAISRAL | |
| | EKEGLKDKKSPLNLSPELQDEIGTAFSLFKTDEDITGRLKDRIQPEILEALLKHISFDKF | |
| | VOISLKALRRIVPLMEQGKRYDEACAEIYGDHYGKKNTEEKIYLPPIPADEIRNPVVLRA | |
| | LSQARKVINGVVRRYGSPARIHIETAREVGKSFKDRKEIEKRQEENRKDREKAAAKFREY | |
| | FPNFVGEPKSKDILKLRLYEQQHGKCLYSGKEINLGRLNEKGYVEIDAALPFSRTWDDSF | |
| | NNKVLVLGSENQNKGNQTPYEYFNGKDNSREWQEFKARVETSRFPRSKKQRILLQKFDED | |
| | GFKERNLNDTRYVNRFLCQFVADRMRLTGKGKKRVFASNGQITNLLRGFWGLRKVRAEND | |
| | RHHALDAVVVACSTVAMQQKITRFVRYKEMNAFDGKTIDKETGEVLHQKTHFPQPWEFFA | |
| | QEVMIRVFGKPDGKPEFEEADTLEKLRTLLAEKLSSRPEAVHEYVTPLFVSRAPNRKMSG | |
| | QGHMETVKSAKRLDEGVSVLRVPLTQLKLKDLEKMVNREREPKLYEALKARLEAHKDDPA | |
| | KAFAEPFYKYDKAGNRTQQVKAVRVEQVQKTGVWVRNHNGIADNATMVRVDVFEKGDKYY | |
| | LVPIYSWQVAKGILPDRAVVQGKDEEDWQLIDDSFNFKFSLHPNDLVEVITKKARMFGYF | |
| | ASCHRGTGNINIRIHDLDHKIGKNGILEGIGVKTALSFQKYQIDELGKEIRPCRLKKRPP | |
| | VR | |
| Cj Cas 9 | MARILAFDIGISSIGWAFSENDELKDCGVRIFTKVENPKTGESLALPRRLARSARKRLAR | 47 |
| C. jejuni | RKARLNHLKHLIANEFKLNYEDYQSFDESLAKAYKGSLISPYELRFRALNELLSKQDFAR | |
| 984 AA | VILHIAKRRGYDDIKNSDDKEKGAILKAIKQNEEKLANYQSVGEYLYKEYFQKFKENSKE | |
| 114.9 kDa | FTNVRNKKESYERCIAQSFLKDELKLIFKKQREFGFSFSKKFEEEVLSVAFYKRALKDFS | |
| | HLVGNCSFFTDEKRAPKNSPLAFMFVALTRIINLLNNLKNTEGILYTKDDLNALLNEVLK | |
| | NGTLTYKQTKKLLGLSDDYEFKGEKGTYFIEFKKYKEFIKALGEHNLSQDDLNEIAKDIT | |
| | LIKDEIKLKKALAKYDLNQNQIDSLSKLEFKDHLNISFKALKLVTPLMLEGKKYDEACNE | |
| | LNLKVAINEDKKDFLPAFNETYYKDEVTNPVVLRAIKEYRKVLNALLKKYGKVHKINIEL | |
| | AREVGKNHSQRAKIEKEQNENYKAKKDAELECEKLGLKINSKNILKLRLFKEQKEFCAYS | |
| | GEKIKISDLQDEKMLEIDHIYPYSRSFDDSYMNKVLVFTKQNQEKLNQTPFEAFGNDSAK | |
| | WQKIEVLAKNLPTKKQKRILDKNYKDKEQKNFKDRNLNDTRYIARLVLNYTKDYLDFLPL | |
| | SDDENTKLNDTQKGSKVHVEAKSGMLTSALRHTWGFSAKDRNNHLHHAIDAVIIAYANNS | |
| | IVKAFSDFKKEQESNSAELYAKKISELDYKNKRKFFEPFSGFRQKVLDKIDEIFVSKPER | |
| | KKPSGALHEETFRKEEEFYQSYGGKEGVLKALELGKIRKVNGKIVKNGDMFRVDIFKHKK | |
| | TNKFYAVPIYTMDFALKVLPNKAVARSKKGEIKDWILMDENYEFCFSLYKDSLILIQTKD | |
| | MQEPEFVYYNAFTSSTVSLIVSKHDNKFETLSKNQKILFKNANEKEVIAKSIGIQNLKVF | |
| | EKYIVSALGEVTKAEFRQREDFKK | |
| GeoCas 9 | MRYKIGLDIGITSVGWAVMNLDIPRIEDLGVRIFDRAENPQTGESLALPRRLARSARRRL | 48 |
| G. | RRRKHRLERIRRLVIREGILTKEELDKLFEEKHEIDVWQLRVEALDRKLNNDELARVLLH | |
| stearothermophilus | LAKRRGFKSNRKSERSNKENSTMLKHIEENRAILSSYRTVGEMIVKDPKFALHKRNKGEN | |
| 1087 AA | YTNTIARDDLEREIRLIFSKQREFGNMSCTEEFENEYITIWASQRPVASKDDIEKKVGFC | |
| 127 kDa | TFEPKEKRAPKATYTFQSFIAWEHINKLRLISPSGARGLTDEERRLLYEQAFQKNKITYH | |
| | DIRTLLHLPDDTYFKGIVYDRGESRKQNENIRFLELDAYHQIRKAVDKVYGKGKSSSFLP | |
| | IDFDTFGYALTLFKDDADIHSYLRNEYEQNGKRMPNLANKVYDNELIEELLNLSFTKFGH | |
| | LSLKALRSILPYMEQGEVYSSACERAGYTFTGPKKKQKTMLLPNIPPIANPVVMRALTQA | |
| | RKVVNAIIKKYGSPVSIHIELARDLSQTFDERRKTKKEQDENRKKNETAIRQLMEYGLTL | |
| | NPTGHDIVKFKLWSEQNGRCAYSLQPIEIERLLEPGYVEVDHVIPYSRSLDDSYTNKVLV | |
| | LTRENREKGNRIPAEYLGVGTERWQQFETFVLTNKQFSKKKRDRLLRLHYDENEETEFKN | |
| | RNLNDTRYISRFFANFIREHLKFAESDDKQKVYTVNGRVTAHLRSRWEFNKNREESDLHH | |
| | AVDAVIVACTTPSDIAKVTAFYQRREQNKELAKKTEPHFPQPWPHFADELRARLSKHPKE | |
| | SIKALNLGNYDDQKLESLQPVFVSRMPKRSVTGAAHQETLRRYVGIDERSGKIQTVVKTK | |
| | LSEIKLDASGHFPMYGKESDPRTYEAIRQRLLEHNNDPKKAFQEPLYKPKKNGEPGPVIR | |
| | TVKIIDTKNQVIPLNDGKTVAYNSNIVRVDVFEKDGKYYCVPVYTMDIMKGILPNKAIEP | |
| | NKPYSEWKEMTEDYTFRFSLYPNDLIRIELPREKTVKTAAGEEINVKDVFVYYKTIDSAN | |
| | GGLELISHDHRFSLRGVGSRTLKRFEKYQVDVLGNIYKVRGEKRVGLASSAHSKPGKTIR | |
| | PLQSTRD | |
| LbaCasl2a | MSKLEKFTNCYSLSKTLRFKAIPVGKTQENIDNKRLLVEDEKRAEDYKGVKKLLDRYYLS | 49 |
| L. bacterium | FINDVLHSIKLKNLNNYISLFRKKTRTEKENKELENLEINLRKEIAKAFKGNEGYKSLFK | |
| 1228 AA | KDIIETILPEFLDDKDEIALVNSFNGFTTAFTGFFDNRENMFSEEAKSTSIAFRCINENL | |
| 143.9 kDa | TRYISNMDIFEKVDAIFDKHEVQEIKEKILNSDYDVEDFFEGEFFNFVLTQEGIDVYNAI | |
| | IGGFVTESGEKIKGLNEYINLYNQKTKQKLPKFKPLYKQVLSDRESLSFYGEGYTSDEEV | |
| | LEVFRNTLNKNSEIFSSIKKLEKLFKNFDEYSSAGIFVKNGPAISTISKDIFGEWNVIRD | |
| | KWNAEYDDIHLKKKAVVTEKYEDDRRKSFKKIGSFSLEQLQEYADADLSVVEKLKEIIIO | |
| | KVDEIYKVYGSSEKLFDADFVLEKSLKKNDAVVAIMKDLLDSVKSFENYIKAFFGEGKET | |
| | NRDESFYGDFVLAYDILLKVDHIYDAIRNYVTQKPYSKDKFKLYFQNPQFMGGWDKDKET | |
| | DYRATILRYGSKYYLAIMDKKYAKCLQKIDKDDVNGNYEKINYKLLPGPNKMLPKVFFSK | |
| | KWMAYYNPSEDIQKIYKNGTFKKGDMFNLNDCHKLIDFFKDSISRYPKWSNAYDFNFSET | |
| | EKYKDIAGFYREVEEQGYKVSFESASKKEVDKLVEEGKLYMFQIYNKDFSDKSHGTPNLH | |
| | TMYFKLLFDENNHGQIRLSGGAELFMRRASLKKEELVVHPANSPIANKNPDNPKKTTTLS | |
| | YDVYKDKRFSEDQYELHIPIAINKCPKNIFKINTEVRVLLKHDDNPYVIGIDRGERNLLY | |
| | IVVVDGKGNIVEQYSLNEIINNFNGIRIKTDYHSLLDKKEKERFEARQNWTSIENIKELK | |
| | AGYISQVVHKICELVEKYDAVIALEDLNSGFKNSRVKVEKQVYQKFEKMLIDKLNYMVDK | |
| | KSNPCATGGALKGYQITNKFESFKSMSTQNGFIFYIPAWLTSKIDPSTGFVNLLKTKYTS | |
| | LADSKKFISSFDRIMYVPEEDLFEFALDYKNFSRTDADYIKKWKLYSYGNRIRIFRNPKK | |
| | NNVFDWEEVCLTSAYKELFNKYGINYQQGDIRALLCEQSDKAFYSSFMALMSLMLQMRNS | |
| | ITGRTDVDFLISPVKNSDGIFYDSRNYEAQENAILPKNADANGAYNIARKVLWAIGQFKK | |
| | AEDEKLDKVKIAISNKEWLEYAOTSVKH | |
| BhCas12b | MATRSFILKIEPNEEVKKGLWKTHEVLNHGIAYYMNILKLIRQEAIYEHHEQDPKNPKKV | 50 |
| B. hisashii | SKAEIQAELWDFVLKMQKCNSFTHEVDKDEVFNILRELYEELVPSSVEKKGEANQLSNKF | |
| 1108 AA | LYPLVDPNSQSGKGTASSGRKPRWYNLKIAGDPSWEEEKKKWEEDKKKDPLAKILGKLAE | |
| 130.4 kDa | YGLIPLFIPYTDSNEPIVKEIKWMEKSRNQSVRRLDKDMFIQALERFLSWESWNLKVKEE | |
| | YEKVEKEYKTLEERIKEDIQALKALEQYEKERQEQLLRDTLNTNEYRLSKRGLRGWREII | |
| | QKWLKMDENEPSEKYLEVFKDYQRKHPREAGDYSVYEFLSKKENHFIWRNHPEYPYLYAT | |
| | FCEIDKKKKDAKQQATFTLADPINHPLWVRFEERSGSNLNKYRILTEQLHTEKLKKKLTV | |
| | QLDRLIYPTESGGWEEKGKVDIVLLPSRQFYNQIFLDIEEKGKHAFTYKDESIKFPLKGT | |
| | LGGARVQFDRDHLRRYPHKVESGNVGRIYFNMTVNIEPTESPVSKSLKIHRDDFPKVVNF | |
| | KPKELTEWIKDSKGKKLKSGIESLEIGLRVMSIDLGQRQAAAASIFEVVDQKPDIEGKLF | |
| | FPIKGTELYAVHRASFNIKLPGETLVKSREVLRKAREDNLKLMNQKLNFLRNVLHFQQFE | |
| | DITEREKRVTKWISRQENSDVPLVYQDELIQIRELMYKPYKDWVAFLKQLHKRLEVEIGK | |
| | EVKHWRKSLSDGRKGLYGISLKNIDEIDRTRKFLLRWSLRPTEPGEVRRLEPGQRFAIDQ | |
| | LNHLNALKEDRLKKMANTIIMHALGYCYDVRKKKWQAKNPACQIILFEDLSNYNPYEERS | |
| | RFENSKLMKWSRREIPRQVALQGEIYGLQVGEVGAQFSSRFHAKTGSPGIRCSVVTKEKL | |
| | QDNRFFKNLQREGRLTLDKIAVLKEGDLYPDKGGEKFISLSKDRKCVTTHADINAAQNLQ | |
| | KRFWTRTHGFYKVYCKAYQVDGQTVYIPESKDQKQKIIEEFGEGYFILKDGVYEWVNAGK | |
| | LKIKKGSSKQSSSELVDSDILKDSFDLASELKGEKLMLYRDPSGNVFPSDKWMAAGVFFG | |
| | KLERILISKLTNQYSISTIEDDSSKQSM | |

(7) Cas9 Equivalents

In some embodiments, the base editors described herein can include any Cas9 equivalent. As used herein, the term “Cas9 equivalent” is a broad term that encompasses any napDNAbp protein that serves the same function as Cas9 in the present base editors despite that its amino acid primary sequence and/or its three-dimensional structure may be different and/or unrelated from an evolutionary standpoint. Thus, while Cas9 equivalents include any Cas9 ortholog, homolog, mutant, or variant described or embraced herein that are evolutionarily related, the Cas9 equivalents also embrace proteins that may have evolved through convergent evolution processes to have the same or similar function as Cas9, but which do not necessarily have any similarity with regard to amino acid sequence and/or three dimensional structure. The base editors described here embrace any Cas9 equivalent that would provide the same or similar function as Cas9 despite that the Cas9 equivalent may be based on a protein that arose through convergent evolution.

For example, CasX is a Cas9 equivalent that reportedly has the same function as Cas9 but which evolved through convergent evolution. Thus, the CasX protein described in Liu et al., “CasX enzymes comprises a distinct family of RNA-guided genome editors,” Nature, 2019, Vol. 566: 218-223, is contemplated to be used with the base editors described herein. In addition, any variant or modification of CasX is conceivable and within the scope of the present disclosure.

Cas9 is a bacterial enzyme that evolved in a wide variety of species. However, the Cas9 equivalents contemplated herein may also be obtained from archaea, which constitute a domain and kingdom of single-celled prokaryotic microbes different from bacteria.

In some embodiments, Cas9 equivalents may refer to CasX or CasY, which have been described in, for example, Burstein et al., “New CRISPR-Cas systems from uncultivated microbes.” Cell Res. 2017 Feb. 21. doi: 10.1038/cr.2017.21, the entire contents of which is hereby incorporated by reference. Using genome-resolved metagenomics, a number of CRISPR-Cas systems were identified, including the first reported Cas9 in the archaeal domain of life. This divergent Cas9 protein was found in little-studied nanoarchaea as part of an active CRISPR-Cas system. In bacteria, two previously unknown systems were discovered, CRISPR-CasX and CRISPR-CasY, which are among the most compact systems yet discovered. In some embodiments, Cas9 refers to CasX, or a variant of CasX. In some embodiments, Cas9 refers to a CasY, or a variant of CasY. It should be appreciated that other RNA-guided DNA binding proteins may be used as a nucleic acid programmable DNA binding protein (napDNAbp), and are within the scope of this disclosure. Also see Liu et al., “CasX enzymes comprises a distinct family of RNA-guided genome editors,” Nature, 2019, Vol. 566: 218-223. Any of these Cas9 equivalents are contemplated.

In some embodiments, the Cas9 equivalent comprises an amino acid sequence that is at least 85%, at least 90%, at least 91%, at least 92%, at least 93%, at least 94%, at least 95%, at least 96%, at least 97%, at least 98%, at least 99%, or at least 99.5% identical to a naturally-occurring CasX or CasY protein. In some embodiments, the napDNAbp is a naturally-occurring CasX or CasY protein. In some embodiments, the napDNAbp comprises an amino acid sequence that is at least 85%, at least 90%, at least 91%, at least 92%, at least 93%, at least 94%, at least 95%, at least 96%, at least 97%, at least 98%, at least 99%, or at least 99.5% identical to a wild-type Cas moiety or any Cas moiety provided herein.

In various embodiments, the nucleic acid programmable DNA binding proteins include, without limitation, Cas9 (e.g., dCas9 and nCas9), CasX, CasY, Cpf1, C2c1, C2c2, C2C3, Argonaute, Cas12a, and Cas12b. One example of a nucleic acid programmable DNA-binding protein that has different PAM specificity than Cas9 is Clustered Regularly Interspaced Short Palindromic Repeats from Prevotella and Francisella 1 (Cpf1). Similar to Cas9, Cpf1 is also a class 2 CRISPR effector. It has been shown that Cpf1 mediates robust DNA interference with features distinct from Cas9. Cpf1 is a single RNA-guided endonuclease lacking tracrRNA, and it utilizes a T-rich protospacer-adjacent motif (TTN, TTTN, or YTN). Moreover, Cpf1 cleaves DNA via a staggered DNA double-stranded break. Out of 16 Cpf1-family proteins, two enzymes from Acidaminococcus and Lachnospiraceae are shown to have efficient genome-editing activity in human cells. Cpf1 proteins are known in the art and have been described previously, for example Yamano et al., “Crystal structure of Cpf1 in complex with guide RNA and target DNA.” Cell (165) 2016, p. 949-962; the entire contents of which is hereby incorporated by reference. The state of the art may also now refer to Cpf1 enzymes as Cas12a.

In still other embodiments, the Cas protein may include any CRISPR associated protein, including but not limited to, Cas12a, Cas12b, Cas1, Cas1B, Cas2, Cas3, Cas4, Cas5, Cas6, Cas7, Cas8, Cas9 (also known as Csn1 and Csx12), Cas10, Csy1, Csy2, Csy3, Cse1, Cse2, Csc1, Csc2, Csa5, Csn2. Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, homologs thereof, or modified versions thereof, and preferably comprising a nickase mutation (e.g., a mutation corresponding to the D10A mutation of the wild type Cas9 polypeptide of SEQ ID NO: 5).

In various other embodiments, the napDNAbp can be any of the following proteins: a Cas9, a Cpf1, a CasX, a CasY, a C2c1, a C2c2, a C2c3, a GeoCas9, a CjCas9, a Cas12a, a Cas12b, a Cas12g, a Cas12h, a Cas12i, a Cas13b, a Cas13c, a Cas13d, a Cas14, a Csn2, an xCas9, an SpCas9-NG, a circularly permuted Cas9, or an Argonaute (Ago) domain, or a variant thereof.

Exemplary Cas9 equivalent protein sequences can include the following:

| Description | Sequence |
|---|---|
| AsCasl2a | MTQFEGFTNLYQVSKTLRFELIPQGKTLKHIQEQGFIEEDKARNDHYKELKPIIDRIYKTYADQCLQLVQLD |
| (previously | WENLSAAIDSYRKEKTEETRNALIEEQATYRNAIHDYFIGRTDNLTDAINKRHAEIYKGLFKAELFNGKVLK |
| known as | QLGTVTTTEHENALLRSFDKFTTYFSGFYENRKNVFSAEDISTAIPHRIVQDNFPKFKENCHIFTRLITAVP |
| Cpfl) | SLREHFENVKKAIGIFVSTSIEEVFSFPFYNQLLTQTQIDLYNQLLGGISREAGTEKIKGLNEVLNLAIQKN |
| Acidaminococcus | DETAHIIASLPHRFIPLFKQILSDRNTLSFILEEFKSDEEVIQSFCKYKTLLRNENVLETAEALFNELNSID |
| sp. | LTHIFISHKKLETISSALCDHWDTLRNALYERRISELTGKITKSAKEKVQRSLKHEDINLQEIISAAGKELS |
| (strain | EAFKQKTSEILSHAHAALDQPLPTTLKKQEEKEILKSQLDSLLGLYHLLDWFAVDESNEVDPEFSARLTGIK |
| BV3L6) | LEMEPSLSFYNKARNYATKKPYSVEKFKLNFQMPTLASGWDVNKEKNNGAILFVKNGLYYLGIMPKQKGRYK |
| UniProtKB | ALSFEPTEKTSEGFDKMYYDYFPDAAKMIPKCSTQLKAVTAHFQTHTTPILLSNNFIEPLEITKEIYDLNNP |
| U2UMQ6 | EKEPKKFQTAYAKKTGDQKGYREALCKWIDFTRDFLSKYTKTTSIDLSSLRPSSQYKDLGEYYAELNPLLYH |
| | ISFQRIAEKEIMDAVETGKLYLFQIYNKDFAKGHHGKPNLHTLYWTGLFSPENLAKTSIKLNGQAELFYRPK |
| | SRMKRMAHRLGEKMLNKKLKDQKTPIPDTLYQELYDYVNHRLSHDLSDEARALLPNVITKEVSHEIIKDRRF |
| | TSDKFFFHVPITLNYQAANSPSKFNQRVNAYLKEHPETPIIGIDRGERNLIYITVIDSTGKILEQRSLNTIQ |
| | QFDYQKKLDNREKERVAARQAWSVVGTIKDLKQGYLSQVIHEIVDLMIHYQAVVVLENLNFGFKSKRTGIAE |
| | KAVYQQFEKMLIDKLNCLVLKDYPAEKVGGVLNPYQLTDQFTSFAKMGTQSGFLFYVPAPYTSKIDPLTGFV |
| | DPFVWKTIKNHESRKHFLEGFDFLHYDVKTGDFILHFKMNRNLSFQRGLPGFMPAWDIVFEKNETQFDAKGT |
| | PFIAGKRIVPVIENHRFTGRYRDLYPANELIALLEEKGIVFRDGSNILPKLLENDDSHAIDTMVALIRSVLQ |
| | MRNSNAATGEDYINSPVRDLNGVCFDSRFQNPEWPMDADANGAYHIALKGQLLLNHLKESKDLKLQNGISNQ |
| | DWLAYIQELRN (SEQ ID NO: 51) |
| AsCasl2a | MTQFEGFTNLYQVSKTLRFELIPQGKTLKHIQEQGFIEEDKARNDHYKELKPIIDRIYKTYADQCLQLVQLD |
| nickase | WENLSAAIDSYRKEKTEETRNALIEEQATYRNAIHDYFIGRTDNLTDAINKRHAEIYKGLFKAELFNGKVLK |
| (e.g., | QLGTVTTTEHENALLRSFDKFTTYFSGFYENRKNVFSAEDISTAIPHRIVQDNFPKFKENCHIFTRLITAVP |
| R1226A) | SLREHFENVKKAIGIFVSTSIEEVFSFPFYNQLLTQTQIDLYNQLLGGISREAGTEKIKGLNEVLNLAIQKN |
| | DETAHIIASLPHRFIPLFKQILSDRNTLSFILEEFKSDEEVIQSFCKYKTLLRNENVLETAEALFNELNSID |
| | LTHIFISHKKLETISSALCDHWDTLRNALYERRISELTGKITKSAKEKVQRSLKHEDINLQEIISAAGKELS |
| | EAFKQKTSEILSHAHAALDQPLPTTLKKQEEKEILKSQLDSLLGLYHLLDWFAVDESNEVDPEFSARLTGIK |
| | LEMEPSLSFYNKARNYATKKPYSVEKFKLNFQMPTLASGWDVNKEKNNGAILFVKNGLYYLGIMPKQKGRYK |
| | ALSFEPTEKTSEGFDKMYYDYFPDAAKMIPKCSTQLKAVTAHFQTHTTPILLSNNFIEPLEITKEIYDLNNP |
| | EKEPKKFQTAYAKKTGDQKGYREALCKWIDFTRDFLSKYTKTTSIDLSSLRPSSQYKDLGEYYAELNPLLYH |
| | ISFQRIAEKEIMDAVETGKLYLFQIYNKDFAKGHHGKPNLHTLYWTGLFSPENLAKTSIKLNGQAELFYRPK |
| | SRMKRMAHRLGEKMLNKKLKDQKTPIPDTLYQELYDYVNHRLSHDLSDEARALLPNVITKEVSHEIIKDRRF |
| | TSDKFFFHVPITLNYQAANSPSKFNQRVNAYLKEHPETPIIGIDRGERNLIYITVIDSTGKILEQRSLNTIQ |
| | QFDYQKKLDNREKERVAARQAWSVVGTIKDLKQGYLSQVIHEIVDLMIHYQAVVVLENLNFGFKSKRTGIAE |
| | KAVYQQFEKMLIDKLNCLVLKDYPAEKVGGVLNPYQLTDQFTSFAKMGTQSGFLFYVPAPYTSKIDPLTGFV |
| | DPFVWKTIKNHESRKHFLEGFDFLHYDVKTGDFILHFKMNRNLSFQRGLPGFMPAWDIVFEKNETQFDAKGT |
| | PFIAGKRIVPVIENHRFTGRYRDLYPANELIALLEEKGIVFRDGSNILPKLLENDDSHAIDTMVALIRSVLQ |
| | MANSNAATGEDYINSPVRDLNGVCFDSRFQNPEWPMDADANGAYHIALKGQLLLNHLKESKDLKLQNGISNQ |
| | DWLAYIQELRN (SEQ ID NQ: 52) |
| LbCasl2a | MNYKTGLEDFIGKESLSKTLRNALIPTESTKIHMEEMGVIRDDELRAEKQQELKEIMDDYYRTFIEEKLGQI |
| (previousl | QGIQWNSLFQKMEETMEDISVRKDLDKIQNEKRKEICCYFTSDKRFKDLFNAKLITDILPNFIKDNKEYTEE |
| y known as | EKAEKEQTRVLFQRFATAFTNYFNQRRNNFSEDNISTAISFRIVNENSEIHLQNMRAFQRIEQQYPEEVCGM |
| Cpfl) | EEEYKDMLQEWQMKHIYSVDFYDRELTQPGIEYYNGICGKINEHMNQFCQKNRINKNDFRMKKLHKQILCKK |
| Lachnospiraceae | SSYYEIPFRFESDQEVYDALNEFIKTMKKKEIIRRCVHLGQECDDYDLGKIYISSNKYEQISNALYGSWDTI |
| bacterium | RKCIKEEYMDALPGKGEKKEEKAEAAAKKEEYRSIADIDKIISLYGSEMDRTISAKKCITEICDMAGQISID |
| GAM79 | PLVCNSDIKLLQNKEKTTEIKTILDSFLHVYQWGQTFIVSDIIEKDSYFYSELEDVLEDFEGITTLYNHVRS |
| Ref Seq. | YVTQKPYSTVKFKLHFGSPTLANGWSQSKEYDNNAILLMRDQKFYLGIFNVRNKPDKQIIKGHEKEEKGDYK |
| WP_1196233 | KMIYNLLPGPSKMLPKVFITSRSGQETYKPSKHILDGYNEKRHIKSSPKFDLGYCWDLIDYYKECIHKHPDW |
| 82.1 | KNYDFHFSDTKDYEDISGFYREVEMQGYQIKWTYISADEIQKLDEKGQIFLFQIYNKDFSVHSTGKDNLHTM |
| | YLKNLFSEENLKDIVLKLNGEAELFFRKASIKTPIVHKKGSVLVNRSYTQTVGNKEIRVSIPEEYYTEIYNY |
| | LNHIGKGKLSSEAQRYLDEGKIKSFTATKDIVKNYRYCCDHYFLHLPITINFKAKSDVAVNERTLAYIAKKE |
| | DIHIIGIDRGERNLLYISVVDVHGNIREQRSFNIVNGYDYQQKLKDREKSRDAARKNWEEIEKIKELKEGYL |
| | SMVIHYIAQLVVKYNAVVAMEDLNYGFKTGRFKVERQVYQKFETMLIEKLHYLVFKDREVCEEGGVLRGYQL |
| | TYIPESLKKVGKQCGFIFYVPAGYTSKIDPTTGFVNLFSFKNLTNRESRQDFVGKFDEIRYDRDKKMFEFSF |
| | DYNNYIKKGTILASTKWKVYTNGTRLKRIVVNGKYTSQSMEVELTDAMEKMLQRAGIEYHDGKDLKGQIVEK |
| | GIEAEIIDIFRLTVQMRNSRSESEDREYDRLISPVLNDKGEFFDTATADKTLPQDADANGAYCIALKGLYEV |
| | KQIKENWKENEQFPRNKLVQDNKTWFDFMQKKRYL (SEQ ID NO: 53) |
| PcCasl2a - | MAKNFEDFKRLYSLSKTLRFEAKPIGATLDNIVKSGLLDEDEHRAASYVKVKKLIDEYHKVFIDRVLDDGCL |
| previously | PLENKGNNNSLAEYYESYVSRAQDEDAKKKFKEIQQNLRSVIAKKLTEDKAYANLFGNKLIESYKDKEDKKK |
| known at | IIDSDLIQFINTAESTQLDSMSQDEAKELVKEFWGFVTYFYGFFDNRKNMYTAEEKSTGIAYRLVNENLPKF |
| Cpfl | IDNIEAFNRAITRPEIQENMGVLYSDFSEYLNVESIQEMFQLDYYNMLLTQKQIDVYNAIIGGKTDDEHDVK |
| Prevotella | IKGINEYINLYNQQHKDDKLPKLKALFKQILSDRNAISWLPEEFNSDQEVLNAIKDCYERLAENVLGDKVLK |
| copri | SLLGSLADYSLDGIFIRNDLQLTDISQKMFGNWGVIQNAIMQNIKRVAPARKHKESEEDYEKRIAGIFKKAD |
| Ref Seq. | SFSISYINDCLNEADPNNAYFVENYFATFGAVNTPTMQRENLFALVQNAYTEVAALLHSDYPTVKHLAQDKA |
| WP_1192277 | NVSKIKALLDAIKSLQHFVKPLLGKGDESDKDERFYGELASLWAELDTVTPLYNMIRNYMTRKPYSQKKIKL |
| 26.1 | NFENPQLLGGWDANKEKDYATIILRRNGLYYLAIMDKDSRKLLGKAMPSDGECYEKMVYKFFKDVTTMIPKC |
| | STQLKDVQAYFKVNTDDYVLNSKAFNKPLTITKEVFDLNNVLYGKYKKFQKGYLTATGDNVGYTHAVNVWIK |
| | FCMDFLNSYDSTCIYDFSSLKPESYLSLDAFYQDANLLLYKLSFARASVSYINQLVEEGKMYLFQIYNKDFS |
| | EYSKGTPNMHTLYWKALFDERNLADVVYKLNGQAEMFYRKKSIENTHPTHPANHPILNKNKDNKKKESLFDY |
| | DLIKDRRYTVDKFMFHVPITMNFKSVGSENINQDVKAYLRHADDMHIIGIDRGERHLLYLVVIDLQGNIKEQ |
| | YSLNEIVNEYNGNTYHTNYHDLLDVREEERLKARQSWQTIENIKELKEGYLSQVIHKITQLMVRYHAIVVLE |
| | DLSKGFMRSRQKVEKQVYQKFEKMLIDKLNYLVDKKTDVSTPGGLLNAYQLTCKSDSSQKLGKQSGFLFYIP |
| | AWNTSKIDPVTGFVNLLDTHSLNSKEKIKAFFSKFDAIRYNKDKKWFEFNLDYDKFGKKAEDTRTKWTLCTR |
| | GMRIDTFRNKEKNSQWDNQEVDLTTEMKSLLEHYYIDIHGNLKDAISAQTDKAFFTGLLHILKLTLQMRNSI |
| | TGTETDYLVSPVADENGIFYDSRSCGNQLPENADANGAYNIARKGLMLIEQIKNAEDLNNVKFDISNKAWLN |
| | FAQQKPYKNG (SEQ ID NO: 54) |
| ErCasl2a - | MFSAKLISDILPEFVIHNNNYSASEKEEKTQVIKLFSRFATSFKDYFKNRANCFSANDISSSSCHRIVNDNA |
| previously | EIFFSNALVYRRIVKNLSNDDINKISGDMKDSLKEMSLEEIYSYEKYGEFITQEGISFYNDICGKVNLFMNL |
| known at | YCQKNKENKNLYKLRKLHKQILCIADTSYEVPYKFESDEEVYQSVNGFLDNISSKHIVERLRKIGENYNGYN |
| Cpfl | LDKIYIVSKFYESVSQKTYRDWETINTALEIHYNNILPGNGKSKADKVKKAVKNDLQKSITEINELVSNYKL |
| Eubacterium | CPDDNIKAETYIHEISHILNNFEAQELKYNPEIHLVESELKASELKNVLDVIMNAFHWCSVFMTEELVDKDN |
| rectale | NFYAELEEIYDEIYPVISLYNLVRNYVTQKPYSTKKIKLNFGIPTLADGWSKSKEYSNNAIILMRDNLYYLG |
| Ref Seq. | IFNAKNKPDKKIIEGNTSENKGDYKKMIYNLLPGPNKMIPKVFLSSKTGVETYKPSAYILEGYKQNKHLKSS |
| WP_1192236 | KDFDITFCHDLIDYFKNCIAIHPEWKNFGFDFSDTSTYEDISGFYREVELQGYKIDWTYISEKDIDLLQEKG |
| 42.1 | QLYLFQIYNKDFSKKSSGNDNLHTMYLKNLFSEENLKDIVLKLNGEAEIFFRKSSIKNPIIHKKGSILVNRT |
| | YEAEEKDQFGNIQIVRKTIPENIYQELYKYFNDKSDKELSDEAAKLKNVVGHHEAATNIVKDYRYTYDKYFL |
| | HMPITINFKANKTSFINDRILQYIAKEKDLHVIGIDRGERNLIYVSVIDTCGNIVEQKSFNIVNGYDYQIKL |
| | KQQEGARQIARKEWKEIGKIKEIKEGYLSLVIHEISKMVIKYNAIIAMEDLSYGFKKGRFKVERQVYQKFET |
| | MLINKLNYLVFKDISITENGGLLKGYQLTYIPDKLKNVGHQCGCIFYVPAAYTSKIDPTTGFVNIFKFKDLT |
| | VDAKREFIKKFDSIRYDSDKNLFCFTFDYNNFITQNTVMSKSSWSVYTYGVRIKRRFVNGRFSNESDTIDIT |
| | KDMEKTLEMTDINWRDGHDLRQDIIDYEIVQHIFEIFKLTVQMRNSLSELEDRDYDRLISPVLNENNIFYDS |
| | AKAGDALPKDADANGAYCIALKGLYEIKQITENWKEDGKFSRDKLKISNKDWFDFIQNKRYL(SEQ ID |
| | NO: 55) |
| CsCas12a - | MNYKTGLEDFIGKESLSKTLRNALIPTESTKIHMEEMGVIRDDELRAEKQQELKEIMDDYYRAFIEEKLGQI |
| previously | QGIQWNSLFQKMEETMEDISVRKDLDKIQNEKRKEICCYFTSDKRFKDLFNAKLITDILPNFIKDNKEYTEE |
| known at | EKAEKEQTRVLFQRFATAFTNYFNQRRNNFSEDNISTAISFRIVNENSEIHLQNMRAFQRIEQQYPEEVCGM |
| Cpfl | EEEYKDMLQEWQMKHIYLVDFYDRVLTQPGIEYYNGICGKINEHMNQFCQKNRINKNDFRMKKLHKQILCKK |
| Clostridium | SSYYEIPFRFESDQEVYDALNEFIKTMKEKEIICRCVHLGQKCDDYDLGKIYISSNKYEQISNALYGSWDTI |
| sp. | RKCIKEEYMDALPGKGEKKEEKAEAAAKKEEYRSIADIDKIISLYGSEMDRTISAKKCITEICDMAGQISTD |
| AF34-10BH | PLVCNSDIKLLQNKEKTTEIKTILDSFLHVYQWGQTFIVSDIIEKDSYFYSELEDVLEDFEGITTLYNHVRS |
| Ref Seq. | YVTQKPYSTVKFKLHFGSPTLANGWSQSKEYDNNAILLMRDQKFYLGIFNVRNKPDKQIIKGHEKEEKGDYK |
| WP_1185384 | KMIYNLLPGPSKMLPKVFITSRSGQETYKPSKHILDGYNEKRHIKSSPKFDLGYCWDLIDYYKECIHKHPDW |
| 18.1 | KNYDFHFSDTKDYEDISGFYREVEMQGYQIKWTYISADEIQKLDEKGQIFLFQIYNKDFSVHSTGKDNLHTM |
| | YLKNLFSEENLKDIVLKLNGEAELFFRKASIKTPVVHKKGSVLVNRSYTQTVGDKEIRVSIPEEYYTEIYNY |
| | LNHIGRGKLSTEAQRYLEERKIKSFTATKDIVKNYRYCCDHYFLHLPITINFKAKSDIAVNERTLAYIAKKE |
| | DIHIIGIDRGERNLLYISVVDVHGNIREQRSFNIVNGYDYQQKLKDREKSRDAARKNWEEIEKIKELKEGYL |
| | SMVIHYIAQLVVKYNAVVAMEDLNYGFKTGRFKVERQVYQKFETMLIEKLHYLVFKDREVCEEGGVLRGYQL |
| | TYIPESLKKVGKQCGFIFYVPAGYTSKIDPTTGFVNLFSFKNLTNRESRQDFVGKFDEIRYDRDKKMFEFSF |
| | DYNNYIKKGTMLASTKWKVYTNGTRLKRIVVNGKYTSQSMEVELTDAMEKMLQRAGIEYHDGKDLKGQIVEK |
| | GIEAEIIDIFRLTVQMRNSRSESEDREYDRLISPVLNDKGEFFDTATADKTLPQDADANGAYCIALKGLYEV |
| | KOIKENWKENEOFPRNKLVODNKTWFDFMOKKRYL (SEQ ID NO: 56) |
| BhCas12b | MATRSFILKIEPNEEVKKGLWKTHEVLNHGIAYYMNILKLIRQEAIYEHHEQDPKNPKKVSKAEIQAELWDF |
| Bacillus | VLKMQKCNSFTHEVDKDEVFNILRELYEELVPSSVEKKGEANQLSNKFLYPLVDPNSQSGKGTASSGRKPRW |
| hisashii | YNLKIAGDPSWEEEKKKWEEDKKKDPLAKILGKLAEYGLIPLFIPYTDSNEPIVKEIKWMEKSRNQSVRRLD |
| Ref Seq. | KDMFIQALERFLSWESWNLKVKEEYEKVEKEYKTLEERIKEDIQALKALEQYEKERQEQLLRDTLNTNEYRL |
| WP 0951425 | SKRGLRGWREIIQKWLKMDENEPSEKYLEVFKDYQRKHPREAGDYSVYEFLSKKENHFIWRNHPEYPYLYAT |
| 15.1 | FCEIDKKKKDAKQQATFTLADPINHPLWVRFEERSGSNLNKYRILTEQLHTEKLKKKLTVQLDRLIYPTESG |
| | GWEEKGKVDIVLLPSRQFYNQIFLDIEEKGKHAFTYKDESIKFPLKGTLGGARVQFDRDHLRRYPHKVESGN |
| | VGRIYFNMTVNIEPTESPVSKSLKIHRDDFPKVVNFKPKELTEWIKDSKGKKLKSGIESLEIGLRVMSIDLG |
| | QRQAAAASIFEVVDQKPDIEGKLFFPIKGTELYAVHRASFNIKLPGETLVKSREVLRKAREDNLKLMNQKLN |
| | FLRNVLHFQQFEDITEREKRVTKWISRQENSDVPLVYQDELIQIRELMYKPYKDWVAFLKQLHKRLEVEIGK |
| | EVKHWRKSLSDGRKGLYGISLKNIDEIDRTRKFLLRWSLRPTEPGEVRRLEPGQRFAIDQLNHLNALKEDRL |
| | KKMANTIIMHALGYCYDVRKKKWQAKNPACQIILFEDLSNYNPYEERSRFENSKLMKWSRREIPRQVALQGE |
| | IYGLQVGEVGAQFSSRFHAKTGSPGIRCSVVTKEKLQDNRFFKNLQREGRLTLDKIAVLKEGDLYPDKGGEK |
| | FISLSKDRKCVTTHADINAAQNLQKRFWTRTHGFYKVYCKAYQVDGQTVYIPESKDQKQKIIEEFGEGYFIL |
| | KDGVYEWVNAGKLKIKKGSSKQSSSELVDSDILKDSFDLASELKGEKLMLYRDPSGNVFPSDKWMAAGVFFG |
| | KLERILISKLTNQYSISTIEDDSSKQSM (SEQ ID NO: 57) |
| ThCas12b | MSEKTTQRAYTLRLNRASGECAVCQNNSCDCWHDALWATHKAVNRGAKAFGDWLLTLRGGLCHTLVEMEVPA |
| Thermomonas | KGNNPPQRPTDQERRDRRVLLALSWLSVEDEHGAPKEFIVATGRDSADDRAKKVEEKLREILEKRDFQEHEI |
| hydrothermalis | DAWLQDCGPSLKAHIREDAVWVNRRALFDAAVERIKTLTWEEAWDFLEPFFGTQYFAGIGDGKDKDDAEGPA |
| Ref Seq. | RQGEKAKDLVQKAGQWLSARFGIGTGADFMSMAEAYEKIAKWASQAQNGDNGKATIEKLACALRPSEPPTLD |
| WP_0727548 | TVLKCISGPGHKSATREYLKTLDKKSTVTQEDLNQLRKLADEDARNCRKKVGKKGKKPWADEVLKDVENSCE |
| 38 | LTYLQDNSPARHREFSVMLDHAARRVSMAHSWIKKAEQRRRQFESDAQKLKNLQERAPSAVEWLDRFCESRS |
| | MTTGANTGSGYRIRKRAIEGWSYVVOAWAEASCDTEDKRIAAARKVOADPEIEKFGDIQLFEALAADEAICV |
| | WRDQEGTQNPSILIDYVTGKTAEHNQKRFKVPAYRHPDELRHPVFCDFGNSRWSIQFAIHKEIRDRDKGAKQ |
| | DTRQLQNRHGLKMRLWNGRSMTDVNLHWSSKRLTADLALDQNPNPNPTEVTRADRLGRAASSAFDHVKIKNV |
| | FNEKEWNGRLQAPRAELDRIAKLEEQGKTEQAEKLRKRLRWYVSFSPCLSPSGPFIVYAGQHNIQPKRSGQY |
| | APHAQANKGRARLAQLILSRLPDLRILSVDLGHRFAAACAVWETLSSDAFRREIQGLNVLAGGSGEGDLFLH |
| | VEMTGDDGKRRTVVYRRIGPDQLLDNTPHPAPWARLDRQFLIKLQGEDEGVREASNEELWTVHKLEVEVGRT |
| | VPLIDRMVRSGFGKTEKQKERLKKLRELGWISAMPNEPSAETDEKEGEIRSISRSVDELMSSALGTLRLALK |
| | RHGNRARIAFAMTADYKPMPGGQKYYFHEAKEASKNDDETKRRDNQIEFLQDALSLWHDLFSSPDWEDNEAK |
| | KLWQNHIATLPNYQTPEEISAELKRVERNKKRKENRDKLRTAAKALAENDQLRQHLHDTWKERWESDDQQWK |
| | ERLRSLKDWIFPRGKAEDNPSIRHVGGLSITRINTISGLYQILKAFKMRPEPDDLRKNIPQKGDDELENFNR |
| | RLLEARDRLREQRVKQLASRIIEAALGVGRIKIPKNGKLPKRPRTTVDTPCHAVVIESLKTYRPDDLRTRRE |
| | NRQLMQWSSAKVRKYLKEGCELYGLHFLEVPANYTSRQCSRTGLPGIRCDDVPTGDFLKAPWWRRAINTARE |
| | KNGGDAKDRFLVDLYDHLNNLQSKGEALPATVRVPRQGGNLFIAGAQLDDTNKERRAIQADLNAAANIGLRA |
| | LLDPDWRGRWWYVPCKDGTSEPALDRIEGSTAFNDVRSLPTGDNSSRRAPREIENLWRDPSGDSLESGTWSP |
| | TRAYWDTVQSRVIELLRRHAGLPTS (SEQ ID NO: 58) |
| LsCas12b | MSIRSFKLKLKTKSGVNAEQLRRGLWRTHQLINDGIAYYMNWLVLLRQEDLFIRNKETNEIEKRSKEEIQAV |
| Laceyella | LLERVHKQQQRNQWSGEVDEQTLLQALRQLYEEIVPSVIGKSGNASLKARFFLGPLVDPNNKTTKDVSKSGP |
| sacchari | TPKWKKMKDAGDPNWVQEYEKYMAERQTLVRLEEMGLIPLFPMYTDEVGDIHWLPQASGYTRTWDRDMFQQA |
| WP_1322218 | IERLLSWESWNRRVRERRAQFEKKTHDFASRFSESDVQWMNKLREYEAQQEKSLEENAFAPNEPYALTKKAL |
| 94.1 | RGWERVYHSWMRLDSAASEEAYWQEVATCQTAMRGEFGDPAIYQFLAQKENHDIWRGYPERVIDFAELNHLQ |
| | RELRRAKEDATFTLPDSVDHPLWVRYEAPGGTNIHGYDLVQDTKRNLTLILDKFILPDENGSWHEVKKVPFS |
| | LAKSKQFHRQVWLQEEQKQKKREVVFYDYSTNLPHLGTLAGAKLQWDRNFLNKRTQQQIEETGEIGKVFFNI |
| | SVDVRPAVEVKNGRLQNGLGKALTVLTHPDGTKIVTGWKAEQLEKWVGESGRVSSLGLDSLSEGLRVMSIDL |
| | GQRTSATVSVFEITKEAPDNPYKFFYQLEGTEMFAVHQRSFLLALPGENPPQKIKQMREIRWKERNRIKQQV |
| | DQLSAILRLHKKVNEDERIQAIDKLLQKVASWQLNEEIATAWNQALSQLYSKAKENDLQWNQAIKNAHHQLE |
| | PVVGKQISLWRKDLSTGRQGIAGLSLWSIEELEATKKLLTRWSKRSREPGVVKRIERFETFAKQIQHHINQV |
| | KENRLKQLANLIVMTALGYKYDQEQKKWIEVYPACQVVLFENLRSYRFSFERSRRENKKLMEWSHRSIPKLV |
| | QMQGELFGLQVADVYAAYSSRYHGRTGAPGIRCHALTEADLRNETNIIHELIEAGFIKEEHRPYLQQGDLVP |
| | WSGGELFATLQKPYDNPRILTLHADINAAQNIQKRFWHPSMWFRVNCESVMEGEIVTYVPKNKTVHKKQGKT |
| | FRFVKVEGSDVYEWAKWSKNRNKNTFSSITERKPPSSMILFRDPSGTFFKEQEWVEQKTFWGKVQSMIQAYM |
| | KKTIVQRMEE (SEQ ID NO: 59) |
| DtCas12b | MVLGRKDDTAELRRALWTTHEHVNLAVAEVERVLLRCRGRSYWTLDRRGDPVHVPESQVAEDALAMAREAQR |
| Dsulfonatronum | RNGWPVVGEDEEILLALRYLYEQIVPSCLLDDLGKPLKGDAQKIGTNYAGPLFDSDTCRRDEGKDVACCGPE |
| thiodismutans | HEVAGKYLGALPEWATPISKQEFDGKDASHLRFKATGGDDAFFRVSIEKANAWYEDPANQDALKNKAYNKDD |
| WP_0313864 | WKKEKDKGISSWAVKYIQKQLQLGQDPRTEVRRKLWLELGLLPLFIPVFDKTMVGNLWNRLAVRLALAHLLS |
| 37 | WESWNHRAVQDQALARAKRDELAALFLGMEDGFAGLREYELRRNESIKQHAFEPVDRPYVVSGRALRSWTRV |
| | REEWLRHGDTQESRKNICNRLQDRLRGKFGDPDVFHWLAEDGQEALWKERDCVTSFSLLNDADGLLEKRKGY |
| | ALMTFADARLHPRWAMYEAPGGSNLRTYQIRKTENGLWADVVLLSPRNESAAVEEKTFNVRLAPSGQLSNVS |
| | FDQIQKGSKMVGRCRYQSANQQFEGLLGGAEILFDRKRIANEQHGATDLASKPGHVWFKLTLDVRPQAPQGW |
| | LDGKGRPALPPEAKHFKTALSNKSKFADQVRPGLRVLSVDLGVRSFAACSVFELVRGGPDQGTYFPAADGRT |
| | VDDPEKLWAKHERSFKITLPGENPSRKEEIARRAAMEELRSLNGDIRRLKAILRLSVLQEDDPRTEHLRLFM |
| | EAIVDDPAKSALNAELFKGFGDDRFRSTPDLWKQHCHFFHDKAEKVVAERFSRWRTETRPKSSSWQDWRERR |
| | GYAGGKSYWAVTYLEAVRGLILRWNMRGRTYGEVNRQDKKQFGTVASALLHHINQLKEDRIKTGADMIIQAA |
| | RGFVPRKNGAGWVQVHEPCRLILFEDLARYRFRTDRSRRENSRLMRWSHREIVNEVGMQGELYGLHVDTTEA |
| | GFSSRYLASSGAPGVRCRHLVEEDFHDGLPGMHLVGELDWLLPKDKDRTANEARRLLGGMVRPGMLVPWDGG |
| | ELFATLNAASQLHVIHADINAAQNLQRRFWGRCGEAIRIVCNQLSVDGSTRYEMAKAPKARLLGALQQLKNG |
| | DAPFHLTSIPNSQKPENSYVMTPTNAGKKYRAGPGEKSSGEEDELALDIVEQAEELAQGRKTFFRDPSGVEE |
| | APDRWLPSEIYWSRIRRRIWQVTLERNSSGRQERAEMDEMPY (SEQ ID NO: 60) |

The base editors described herein may also comprise Cas12a/Cpf1 (dCpf1) variants that may be used as a guide nucleotide sequence-programmable DNA-binding protein domain. The Cas12a/Cpf1 protein has a RuvC-like endonuclease domain that is similar to the RuvC domain of Cas9 but does not have a HNH endonuclease domain, and the N-terminal of Cpf1 does not have the alfa-helical recognition lobe of Cas9. It was shown in Zetsche et al., Cell, 163, 759-771, 2015 (which is incorporated herein by reference) that, the RuvC-like domain of Cpf1 is responsible for cleaving both DNA strands and inactivation of the RuvC-like domain inactivates Cpf1 nuclease activity.

(8) Cas9 Equivalents with Expanded PAM Sequence

In some embodiments, the napDNAbp is a nucleic acid programmable DNA binding protein that does not require a canonical (NGG) PAM sequence. In some embodiments, the napDNAbp is an argonaute protein. One example of such a nucleic acid programmable DNA binding protein is an Argonaute protein from Natronobacterium gregoryi (NgAgo). NgAgo is a ssDNA-guided endonuclease. NgAgo binds 5′ phosphorylated ssDNA of ˜24 nucleotides (gDNA) to guide it to its target site and will make DNA double-strand breaks at the gDNA site. In contrast to Cas9, the NgAgo-gDNA system does not require a protospacer-adjacent motif (PAM). Using a nuclease inactive NgAgo (dNgAgo) can greatly expand the bases that may be targeted. The characterization and use of NgAgo have been described in Gao et al., Nat Biotechnol., 2016 July; 34(7):768-73. PubMed PMID: 27136078; Swarts et al., Nature. 507(7491) (2014):258-61; and Swarts et al., Nucleic Acids Res. 43(10) (2015):5120-9, each of which is incorporated herein by reference.

In some embodiments, the napDNAbp is a prokaryotic homolog of an Argonaute protein. Prokaryotic homologs of Argonaute proteins are known and have been described, for example, in Makarova K., et al., “Prokaryotic homologs of Argonaute proteins are predicted to function as key components of a novel system of defense against mobile genetic elements”, Biol Direct. 2009 Aug. 25; 4:29. doi: 10.1186/1745-6150-4-29, the entire contents of which is hereby incorporated by reference. In some embodiments, the napDNAbp is a Marinitoga piezophila Argunaute (MpAgo) protein. The CRISPR-associated Marinitoga piezophila Argunaute (MpAgo) protein cleaves single-stranded target sequences using 5′-phosphorylated guides. The 5′ guides are used by all known Argonautes. The crystal structure of an MpAgo-RNA complex shows a guide strand binding site comprising residues that block 5′ phosphate interactions. This data suggests the evolution of an Argonaute subclass with noncanonical specificity for a 5′-hydroxylated guide. See, e.g., Kaya et al., “A bacterial Argonaute with noncanonical guide RNA specificity”, Proc Natl Acad Sci USA. 2016 Apr. 12; 113(15):4057-62, the entire contents of which are hereby incorporated by reference). It should be appreciated that other argonaute proteins may be used, and are within the scope of this disclosure.

In some embodiments, the napDNAbp is a single effector of a microbial CRISPR-Cas system. Single effectors of microbial CRISPR-Cas systems include, without limitation, Cas9, Cpf1, C2c1, C2c2, and C2c3. Typically, microbial CRISPR-Cas systems are divided into Class 1 and Class 2 systems. Class 1 systems have multisubunit effector complexes, while Class 2 systems have a single protein effector. For example, Cas9 and Cpf1 are Class 2 effectors. In addition to Cas9 and Cpf1, three distinct Class 2 CRISPR-Cas systems (C2c1, C2c2, and C2c3) have been described by Shmakov et al., “Discovery and Functional Characterization of Diverse Class 2 CRISPR Cas Systems”, Mol. Cell, 2015 Nov. 5; 60(3): 385-397, the entire contents of which is hereby incorporated by reference. Effectors of two of the systems, C2c1 and C2c3, contain RuvC-like endonuclease domains related to Cpf1. A third system, C2c2 contains an effector with two predicated HEPN RNase domains. Production of mature CRISPR RNA is tracrRNA-independent, unlike production of CRISPR RNA by C2c1. C2c1 depends on both CRISPR RNA and tracrRNA for DNA cleavage. Bacterial C2c2 has been shown to possess a unique RNase activity for CRISPR RNA maturation distinct from its RNA-activated single-stranded RNA degradation activity. These RNase functions are different from each other and from the CRISPR RNA-processing behavior of Cpf1. See, e.g., East-Seletsky, et al., “Two distinct RNase activities of CRISPR-C2c2 enable guide-RNA processing and RNA detection”, Nature, 2016 Oct. 13; 538(7624):270-273, the entire contents of which are hereby incorporated by reference. In vitro biochemical analysis of C2c2 in Leptotrichia shahii has shown that C2c2 is guided by a single CRISPR RNA and can be programed to cleave ssRNA targets carrying complementary protospacers. Catalytic residues in the two conserved HEPN domains mediate cleavage. Mutations in the catalytic residues generate catalytically inactive RNA-binding proteins. See e.g., Abudayyeh et al., “C2c2 is a single-component programmable RNA-guided RNA-targeting CRISPR effector”, Science, 2016 Aug. 5; 353(6299), the entire contents of which are hereby incorporated by reference.

The crystal structure of Alicyclobaccillus acidoterrastris C2c1 (AacC2c1) has been reported in complex with a chimeric single-molecule guide RNA (sgRNA). See e.g., Liu et al., “C2c1-sgRNA Complex Structure Reveals RNA-Guided DNA Cleavage Mechanism”, Mol. Cell, 2017 Jan. 19; 65(2):310-322, the entire contents of which are hereby incorporated by reference. The crystal structure has also been reported in Alicyclobacillus acidoterrestris C2c1 bound to target DNAs as ternary complexes. See e.g., Yang et al., “PAM-dependent Target DNA Recognition and Cleavage by C2C1 CRISPR-Cas endonuclease”, Cell, 2016 Dec. 15; 167(7):1814-1828, the entire contents of which are hereby incorporated by reference. Catalytically competent conformations of AacC2c1, both with target and non-target DNA strands, have been captured independently positioned within a single RuvC catalytic pocket, with C2c1-mediated cleavage resulting in a staggered seven-nucleotide break of target DNA. Structural comparisons between C2c1 ternary complexes and previously identified Cas9 and Cpf1 counterparts demonstrate the diversity of mechanisms used by CRISPR-Cas9 systems.

In some embodiments, the napDNAbp may be a C2c1, a C2c2, or a C2c3 protein. In some embodiments, the napDNAbp is a C2c1 protein. In some embodiments, the napDNAbp is a C2c2 protein. In some embodiments, the napDNAbp is a C2c3 protein. In some embodiments, the napDNAbp comprises an amino acid sequence that is at least 85%, at least 90%, at least 91%, at least 92%, at least 93%, at least 94%, at least 95%, at least 96%, at least 97%, at least 98%, at least 99%, or at least 99.5% identical to a naturally-occurring C2c1, C2c2, or C2c3 protein. In some embodiments, the napDNAbp is a naturally-occurring C2c1, C2c2, or C2c3 protein.

Some aspects of the disclosure provide Cas9 domains that have different PAM specificities. Typically, Cas9 proteins, such as Cas9 from S. pyogenes (spCas9), require a canonical NGG PAM sequence to bind a particular nucleic acid region. This may limit the ability to edit desired bases within a genome. In some embodiments, the base editing fusion proteins provided herein may need to be placed at a precise location, for example where a target base is placed within a 4 base region (e.g., a “editing window”), which is approximately 15 bases upstream of the PAM. See Komor, A. C., et al., “Programmable editing of a target base in genomic DNA without double-stranded DNA cleavage” Nature 533, 420-424 (2016), the entire contents of which are hereby incorporated by reference. Accordingly, in some embodiments, any of the fusion proteins provided herein may contain a Cas9 domain that is capable of binding a nucleotide sequence that does not contain a canonical (e.g., NGG) PAM sequence. Cas9 domains that bind to non-canonical PAM sequences have been described in the art and would be apparent to the skilled artisan. For example, Cas9 domains that bind non-canonical PAM sequences have been described in Kleinstiver, B. P., et al., “Engineered CRISPR-Cas9 nucleases with altered PAM specificities” Nature 523, 481-485 (2015); and Kleinstiver, B. P., et al., “Broadening the targeting range of Staphylococcus aureus CRISPR-Cas9 by modifying PAM recognition” Nature Biotechnology 33, 1293-1298 (2015); the entire contents of each are hereby incorporated by reference.

For example, a napDNAbp domain with altered PAM specificity, such as a domain with at least 80%, at least 85%, at least 90%, at least 95%, or at least 99% sequence identity with wild type Francisella novicida Cpf1 (SEQ ID NO: 61) (D917, E1006, and D1255), which has the following amino acid sequence:

(SEQ ID NO: 61)
MSIYQEFVNKYSLSKTLRFELIPQGKTLENIKARG
LILDDEKRAKDYKKAKQIIDKYHQFFIEEILSSVC
ISEDLLQNYSDVYFKLKKSDDDNLQKDFKSAKDTI
KKQISEYIKDSEKFKNLFNQNLIDAKKGQESDLIL
WLKQSKDNGIELFKANSDITDIDEALEIIKSFKGW
TTYFKGFHENRKNVYSSNDIPTSIIYRIVDDNLPK
FLENKAKYESLKDKAPEAINYEQIKKDLAEELTFD
IDYKTSEVNQRVFSLDEVFEIANFNNYLNQSGITK
FNTIIGGKFVNGENTKRKGINEYINLYSQQINDKT
LKKYKMSVLFKQILSDTESKSFVIDKLEDDSDVVT
TMQSFYEQIAAFKTVEEKSIKETLSLLFDDLKAQK
LDLSKIYFKNDKSLTDLSQQVFDDYSVIGTAVLEY
ITQQIAPKNLDNPSKKEQELIAKKTEKAKYLSLET
IKLALEEFNKHRDIDKQCRFEEILANFAAIPMIFD
EIAQNKDNLAQISIKYQNQGKKDLLQASAEDDVKA
IKDLLDQTNNLLHKLKIFHISQSEDKANILDKDEH
FYLVFEECYFELANIVPLYNKIRNYITQKPYSDEK
FKLNFENSTLANGWDKNKEPDNTAILFIKDDKYYL
GVMNKKNNKIFDDKAIKENKGEGYKKIVYKLLPGA
NKMLPKVFFSAKSIKFYNPSEDILRIRNHSTHTKN
GSPQKGYEKFEFNIEDCRKFIDFYKQSISKHPEWK
DFGFRFSDTQRYNSIDEFYREVENQGYKLTFENIS
ESYIDSVVNQGKLYLFQIYNKDFSAYSKGRPNLHT
LYWKALFDERNLQDVVYKLNGEAELFYRKQSIPKK
ITHPAKEAIANKNKDNPKKESVFEYDLIKDKRFTE
DKFFFHCPITINFKSSGANKFNDEINLLLKEKAND
VHILSIDRGERHLAYYTLVDGKGNIIKQDTFNIIG
NDRMKTNYHDKLAAIEKDRDSARKDWKKINNIKEM
KEGYLSQVVHEIAKLVIEYNAIVVFEDLNFGFKRG
RFKVEKQVYQKLEKMLIEKLNYLVFKDNEFDKTGG
VLRAYQLTAPFETFKKMGKQTGIIYYVPAGFTSKI
CPVTGFVNQLYPKYESVSKSQEFFSKFDKICYNLD
KGYFEFSFDYKNFGDKAAKGKWTIASFGSRLINFR
NSDKNHNWDTREVYPTKELEKLLKDYSIEYGHGEC
IKAAICGESDKKFFAKLTSVLNTILQMRNSKTGTE
LDYLISPVADVNGNFFDSRQAPKNMPQDADANGAY
HIGLKGLMLLGRIKNNQEGKKLNLVIKNEEYFEFV
QNRNN

An additional napDNAbp domain with altered PAM specificity, such as a domain having at least 80%, at least 85%, at least 90%, at least 95%, or at least 99% sequence identity with wild type Geobacillus thermodenitrificans Cas9 (SEQ ID NO: 62), which has the following amino acid sequence:

(SEQ ID NO: 62)
MKYKIGLDIGITSIGWAVINLDIPRIEDLGVRIFD
RAENPKTGESLALPRRLARSARRRLRRRKHRLERI
RRLFVREGILTKEELNKLFEKKHEIDVWQLRVEAL
DRKLNNDELARILLHLAKRRGFRSNRKSERTNKEN
STMLKHIEENQSILSSYRTVAEMVVKDPKFSLHKR
NKEDNYTNTVARDDLEREIKLIFAKQREYGNIVCT
EAFEHEYISIWASQRPFASKDDIEKKVGFCTFEPK
EKRAPKATYTFQSFTVWEHINKLRLVSPGGIRALT
DDERRLIYKQAFHKNKITFHDVRTLLNLPDDTRFK
GLLYDRNTTLKENEKVRFLELGAYHKIRKAIDSVY
GKGAAKSFRPIDFDTFGYALTMFKDDTDIRSYLRN
EYEQNGKRMENLADKVYDEELIEELLNLSFSKFGH
LSLKALRNILPYMEQGEVYSTACERAGYTFTGPKK
KQKTVLLPNIPPIANPVVMRALTQARKVVNAIIKK
YGSPVSIHIELARELSQSFDERRKMQKEQEGNRKK
NETAIRQLVEYGLTLNPTGLDIVKFKLWSEQNGKC
AYSLQPIEIERLLEPGYTEVDHVIPYSRSLDDSYT
NKVLVLTKENREKGNRTPAEYLGLGSERWQQFETF
VLTNKQFSKKKRDRLLRLHYDENEENEFKNRNLND
TRYISRFLANFIREHLKFADSDDKQKVYTVNGRIT
AHLRSRWNFNKNREESNLHHAVDAAIVACTTPSDI
ARVTAFYQRREQNKELSKKTDPQFPQPWPHFADEL
QARLSKNPKESIKALNLGNYDNEKLESLQPVFVSR
MPKRSITGAAHQETLRRYIGIDERSGKIQTVVKKK
LSEIQLDKTGHFPMYGKESDPRTYEAIRQRLLEHN
NDPKKAFQEPLYKPKKNGELGPIIRTIKIIDTTNQ
VIPLNDGKTVAYNSNIVRVDVFEKDGKYYCVPIYT
IDMMKGILPNKAIEPNKPYSEWKEMTEDYTFRFSL
YPNDLIRIEFPREKTIKTAVGEEIKIKDLFAYYQT
IDSSNGGLSLVSHDNNFSLRSIGSRTLKRFEKYQV
DVLGNIYKVRGEKRVGVASSSHSKAGETIRPL

In some embodiments, the nucleic acid programmable DNA binding protein (napDNAbp) is a nucleic acid programmable DNA binding protein that does not require a canonical (NGG) PAM sequence. In some embodiments, the napDNAbp is an argonaute protein. One example of such a nucleic acid programmable DNA binding protein is an Argonaute protein from Natronobacterium gregoryi (NgAgo). NgAgo is a ssDNA-guided endonuclease. NgAgo binds 5′ phosphorylated ssDNA of ˜24 nucleotides (gDNA) to guide it to its target site and will make DNA double-strand breaks at the gDNA site. In contrast to Cas9, the NgAgo-gDNA system does not require a protospacer-adjacent motif (PAM). Using a nuclease inactive NgAgo (dNgAgo) can greatly expand the bases that may be targeted. The characterization and use of NgAgo have been described in Gao et al., Nat Biotechnol., 34(7): 768-73 (2016), PubMed PMID: 27136078; Swarts et al., Nature, 507(7491): 258-61 (2014); and Swarts et al., Nucleic Acids Res. 43(10) (2015): 5120-9, each of which is incorporated herein by reference. The sequence of Natronobacterium gregoryi Argonaute is provided in SEQ ID NO: 63.

The disclosed fusion proteins may comprise a napDNAbp domain having at least 80%, at least 85%, at least 90%, at least 95%, or at least 99% sequence identity with wild type Natronobacterium gregoryi Argonaute (SEQ ID NO: 63), which has the following amino acid sequence:

(SEQ ID NO: 63)
MTVIDLDSTTTADELTSGHTYDISVTLTGVYDNTD
EQHPRMSLAFEQDNGERRYITLWKNTTPKDVFTYD
YATGSTYIFTNIDYEVKDGYENLTATYQTTVENAT
AQEVGTTDEDETFAGGEPLDHHLDDALNETPDDAE
TESDSGHVMTSFASRDQLPEWTLHTYTLTATDGAK
TDTEYARRTLAYTVRQELYTDHDAAPVATDGLMLL
TPEPLGETPLDLDCGVRVEADETRTLDYTTAKDRL
LARELVEEGLKRSLWDDYLVRGIDEVLSKEPVLTC
DEFDLHERYDLSVEVGHSGRAYLHINFRHRFVPKL
TLADIDDDNIYPGLRVKTTYRPRRGHIVWGLRDEC
ATDSLNTLGNQSVVAYHRNNQTPINTDLLDAIEAA
DRRVVETRRQGHGDDAVSFPQELLAVEPNTHQIKQ
FASDGFHQQARSKTRLSASRCSEKAQAFAERLDPV
RLNGSTVEFSSEFFTGNNEQQLRLLYENGESVLTF
RDGARGAHPDETFSKGIVNPPESFEVAVVLPEQQA
DTCKAQWDTMADLLNQAGAPPTRSETVQYDAFSSP
ESISLNVAGAIDPSEVDAAFVVLPPDQEGFADLAS
PTETYDELKKALANMGIYSQMAYFDRFRDAKIFYT
RNVALGLLAAAGGVAFTTEHAMPGDADMFIGIDVS
RSYPEDGASGQINIAATATAVYKDGTILGHSSTRP
QLGEKLQSTDVRDIMKNAILGYQQVTGESPTHIVI
HRDGFMNEDLDPATEFLNEQGVEYDIVEIRKQPQT
RLLAVSDVQYDTPVKSIAAINQNEPRATVATFGAP
EYLATRDGGGLPRPIQIERVAGETDIETLTRQVYL
LSQSHIQVHNSTARLPITTAYADQASTHATKGYLV
QTGAFESNVGFL

(9) Cas9 Circular Permutants

In various embodiments, the base editors disclosed herein may comprise a circular permutant of Cas9.

The term “circularly permuted Cas9” or “circular permutant” of Cas9 or “CP-Cas9”) refers to any Cas9 protein, or variant thereof, that occurs or has been modify to engineered as a circular permutant variant, which means the N-terminus and the C-terminus of a Cas9 protein (e.g., a wild type Cas9 protein) have been topically rearranged. Such circularly permuted Cas9 proteins, or variants thereof, retain the ability to bind DNA when complexed with a guide RNA (gRNA). See, Oakes et al., “Protein Engineering of Cas9 for enhanced function,” Methods Enzymol, 2014, 546: 491-511 and Oakes et al., “CRISPR-Cas9 Circular Permutants as Programmable Scaffolds for Genome Modification,” Cell, Jan. 10, 2019, 176: 254-267, each of are incorporated herein by reference. The instant disclosure contemplates any previously known CP-Cas9 or use a new CP-Cas9 so long as the resulting circularly permuted protein retains the ability to bind DNA when complexed with a guide RNA (gRNA).

Any of the Cas9 proteins described herein, including any variant, ortholog, or naturally occurring Cas9 or equivalent thereof, may be reconfigured as a circular permutant variant.

In various embodiments, the circular permutants of Cas9 may have the following structure: N-terminus-[original C-terminus]-[optional linker]-[original N-terminus]-C-terminus.

As an example, the present disclosure contemplates the following circular permutants of canonical S. pyogenes Cas9 (1368 amino acids of UniProtKB-Q99ZW2 (CAS9_STRP1) (numbering is based on the amino acid position in SEQ ID NO: 5)): N-terminus-[1268-1368]-[optional linker]-[1-1267]-C-terminus; N-terminus-[1168-1368]-[optional linker]-[1-1167]-C-terminus; N-terminus-[1068-1368]-[optional linker]-[1-1067]-C-terminus; N-terminus-[968-1368]-[optional linker]-[1-967]-C-terminus; N-terminus-[868-1368]-[optional linker]-[1-867]-C-terminus; N-terminus-[768-1368]-[optional linker]-[1-767]-C-terminus; N-terminus-[668-1368]-[optional linker]-[1-667]-C-terminus; N-terminus-[568-1368]-[optional linker]-[1-567]-C-terminus; N-terminus-[468-1368]-[optional linker]-[1-467]-C-terminus; N-terminus-[368-1368]-[optional linker]-[1-367]-C-terminus; N-terminus-[268-1368]-[optional linker]-[1-267]-C-terminus; N-terminus-[168-1368]-[optional linker]-[1-167]-C-terminus; N-terminus-[68-1368]-[optional linker]-[1-67]-C-terminus; or N-terminus-[10-1368]-[optional linker]-[1-9]-C-terminus, or the corresponding circular permutants of other Cas9 proteins (including other Cas9 orthologs, variants, etc).

In particular embodiments, the circular permutant Cas9 has the following structure (based on S. pyogenes Cas9 (1368 amino acids of UniProtKB—Q99ZW2 (CAS9_STRP1) (numbering is based on the amino acid position in SEQ ID NO: 5): N-terminus-[102-1368]-[optional linker]-[1-101]-C-terminus; N-terminus-[1028-1368]-[optional linker]-[1-1027]-C-terminus; N-terminus-[1041-1368]-[optional linker]-[1-1043]-C-terminus; N-terminus-[1249-1368]-[optional linker]-[1-1248]-C-terminus; or N-terminus-[1300-1368]-[optional linker]-[1-1299]-C-terminus, or the corresponding circular permutants of other Cas9 proteins (including other Cas9 orthologs, variants, etc).

In still other embodiments, the circular permutant Cas9 has the following structure (based on S. pyogenes Cas9 (1368 amino acids of UniProtKB—Q99ZW2 (CAS9_STRP1) (numbering is based on the amino acid position in SEQ ID NO: 5): N-terminus-[103-1368]-[optional linker]-[1-102]-C-terminus; N-terminus-[1029-1368]-[optional linker]-[1-1028]-C-terminus; N-terminus-[1042-1368]-[optional linker]-[1-1041]-C-terminus; N-terminus-[1250-1368]-[optional linker]-[1-1249]-C-terminus; or N-terminus-[1301-1368]-[optional linker]-[1-1300]-C-terminus, or the corresponding circular permutants of other Cas9 proteins (including other Cas9 orthologs, variants, etc).

In some embodiments, the circular permutant can be formed by linking a C-terminal fragment of a Cas9 to an N-terminal fragment of a Cas9, either directly or by using a linker, such as an amino acid linker. In some embodiments, The C-terminal fragment may correspond to the C-terminal 95% or more of the amino acids of a Cas9 (e.g., amino acids about 1300-1368), or the C-terminal 90%, 85%, 80%, 75%, 70%, 65%, 60%, 55%, 50%, 45%, 40%, 35%, 30%, 25%, 20%, 15%, 10%, or 5% or more of a Cas9 (e.g., any one of SEQ ID NOs: 5, 8, 10, 12-26). The N-terminal portion may correspond to the N-terminal 95% or more of the amino acids of a Cas9 (e.g., amino acids about 1-1300), or the N-terminal 90%, 85%, 80%, 75%, 70%, 65%, 60%, 55%, 50%, 45%, 40%, 35%, 30%, 25%, 20%, 15%, 10%, or 5% or more of a Cas9 (e.g., of SEQ ID NO: 5, 8, 10, 12-26).

In some embodiments, the circular permutant can be formed by linking a C-terminal fragment of a Cas9 to an N-terminal fragment of a Cas9, either directly or by using a linker, such as an amino acid linker. In some embodiments, the C-terminal fragment that is rearranged to the N-terminus, includes or corresponds to the C-terminal 30% or less of the amino acids of a Cas9 (e.g., amino acids 1012-1368 of SEQ ID NO: 5). In some embodiments, the C-terminal fragment that is rearranged to the N-terminus, includes or corresponds to the C-terminal 30%, 29%, 28%, 27%, 26%, 25%, 24%, 23%, 22%, 21%, 20%, 19%, 18%, 17%, 16%, 15%, 14%, 13%, 12%, 11%, 10%, 9%, 8%, 7%, 6%, 5%, 4%, 3%, 2%, or 1% of the amino acids of a Cas9 (e.g., the Cas9 of SEQ ID NO: 5). In some embodiments, the C-terminal fragment that is rearranged to the N-terminus, includes or corresponds to the C-terminal 410 residues or less of a Cas9 (e.g., the Cas9 of SEQ ID NO: 5). In some embodiments, the C-terminal portion that is rearranged to the N-terminus, includes or corresponds to the C-terminal 410, 400, 390, 380, 370, 360, 350, 340, 330, 320, 310, 300, 290, 280, 270, 260, 250, 240, 230, 220, 210, 200, 190, 180, 170, 160, 150, 140, 130, 120, 110, 100, 90, 80, 70, 60, 50, 40, 30, 20, or 10 residues of a Cas9 (e.g., the Cas9 of SEQ ID NO: 5). In some embodiments, the C-terminal portion that is rearranged to the N-terminus, includes or corresponds to the C-terminal 357, 341, 328, 120, or 69 residues of a Cas9 (e.g., the Cas9 of SEQ ID NO: 5).

In other embodiments, circular permutant Cas9 variants may be defined as a topological rearrangement of a Cas9 primary structure based on the following method, which is based on S. pyogenes Cas9 of SEQ ID NO: 5: (a) selecting a circular permutant (CP) site corresponding to an internal amino acid residue of the Cas9 primary structure, which dissects the original protein into two halves: an N-terminal region and a C-terminal region; (b) modifying the Cas9 protein sequence (e.g., by genetic engineering techniques) by moving the original C-terminal region (comprising the CP site amino acid) to precede the original N-terminal region, thereby forming a new N-terminus of the Cas9 protein that now begins with the CP site amino acid residue. The CP site can be located in any domain of the Cas9 protein, including, for example, the helical-II domain, the RuvCIII domain, or the CTD domain. For example, the CP site may be located (relative the S. pyogenes Cas9 of SEQ ID NO: 5) at original amino acid residue 181, 199, 230, 270, 310, 1010, 1016, 1023, 1029, 1041, 1247, 1249, or 1282. Thus, once relocated to the N-terminus, original amino acid 181, 199, 230, 270, 310, 1010, 1016, 1023, 1029, 1041, 1247, 1249, or 1282 would become the new N-terminal amino acid. Nomenclature of these CP-Cas9 proteins may be referred to as Cas9-CP¹⁸¹, Cas9-CP¹⁹⁹, Cas9-CP²³⁰, Cas9-CP²⁷⁰, Cas9-CP³¹⁰, Cas9-CP¹⁰¹⁰, Cas9-CP¹⁰¹⁶, Cas9-CP¹⁰²³, Cas9-CP¹⁰²⁹, Cas9-CP¹⁰⁴¹, Cas9-CP¹²⁴⁷, Cas9-CP¹²⁴⁹, and Cas9-CP¹²⁸², respectively. This description is not meant to be limited to making CP variants from SEQ ID NO: 5, but may be implemented to make CP variants in any Cas9 sequence, either at CP sites that correspond to these positions, or at other CP sites entirely. This description is not meant to limit the specific CP sites in any way. Virtually any CP site may be used to form a CP-Cas9 variant.

Exemplary CP-Cas9 amino acid sequences, based on the Cas9 of SEQ ID NO: 5, are provided below in which linker sequences are indicated by underlining and optional methionine (M) residues are indicated in bold. It should be appreciated that the disclosure provides CP-Cas9 sequences that do not include a linker sequence or that include different linker sequences. It should be appreciated that CP-Cas9 sequences may be based on Cas9 sequences other than that of SEQ ID NO: 5 and any examples provided herein are not meant to be limiting. Exemplary CP-Cas9 sequences are as follows:

| CP name | Sequence | SEQ ID NO: |
|---|---|---|
| CP1012 | DYKVYDVRKMIAKSEQEIGKATAKYFFYSNIMNFFKTEITLANGEIRKRPLIETN | SEQ ID NO: 64 |
| | GETGEIVWDKGRDFATVRKVLSMPQVNIVKKTEVQTGGFSKESILPKRNSDKLIA | |
| | RKKDWDPKKYGGFDSPTVAYSVLVVAKVEKGKSKKLKSVKELLGITIMERSSFEK | |
| | NPIDFLEAKGYKEVKKDLIIKLPKYSLFELENGRKRMLASAGELQKGNELALPSK | |
| | YVNFLYLASHYEKLKGSPEDNEQKQLFVEQHKHYLDEIIEQISEFSKRVILADAN | |
| | LDKVLSAYNKHRDKPIREQAENIIHLFTLTNLGAPAAFKYFDTTIDRKRYTSTKE | |
| | VLDATLIHQSITGLYETRIDLSQLGGDGGSGGSGGSGGSGGSGGSGGDKKYSIGL | |
| | AIGTNSVGWAVITDEYKVPSKKFKVLGNTDRHSIKKNLIGALLFDSGETAEATRL | |
| | KRTARRRYTRRKNRICYLQEIFSNEMAKVDDSFFHRLEESFLVEEDKKHERHPIF | |
| | GNIVDEVAYHEKYPTIYHLRKKLVDSTDKADLRLIYLALAHMIKFRGHFLIEGDL | |
| | NPDNSDVDKLFIQLVQTYNQLFEENPINASGVDAKAILSARLSKSRRLENLIAQL | |
| | PGEKKNGLFGNLIALSLGLTPNFKSNFDLAEDAKLQLSKDTYDDDLDNLLAQIGD | |
| | QYADLFLAAKNLSDAILLSDILRVNTEITKAPLSASMIKRYDEHHQDLTLLKALV | |
| | RQQLPEKYKEIFFDQSKNGYAGYIDGGASQEEFYKFIKPILEKMDGTEELLVKLN | |
| | REDLLRKQRTFDNGSIPHQIHLGELHAILRRQEDFYPFLKDNREKIEKILTFRIP | |
| | YYVGPLARGNSRFAWMTRKSEETITPWNFEEVVDKGASAQSFIERMTNFDKNLPN | |
| | EKVLPKHSLLYEYFTVYNELTKVKYVTEGMRKPAFLSGEQKKAIVDLLFKTNRKV | |
| | TVKQLKEDYFKKIECFDSVEISGVEDRFNASLGTYHDLLKIIKDKDFLDNEENED | |
| | ILEDIVLTLTLFEDREMIEERLKTYAHLFDDKVMKQLKRRRYTGWGRLSRKLING | |
| | IRDKQSGKTILDFLKSDGFANRNFMQLIHDDSLTFKEDIQKAQVSGQGDSLHEHI | |
| | ANLAGSPAIKKGILQTVKVVDELVKVMGRHKPENIVIEMARENQTTQKGQKNSRE | |
| | RMKRIEEGIKELGSQILKEHPVENTQLQNEKLYLYYLQNGRDMYVDQELDINRLS | |
| | DYDVDHIVPQSFLKDDSIDNKVLTRSDKNRGKSDNVPSEEVVKKMKNYWRQLLNA | |
| | KLITQRKFDNLTKAERGGLSELDKAGFIKRQLVETRQITKHVAQILDSRMNTKYD | |
| | ENDKLIREVKVITLKSKLVSDFRKDFQFYKVREINNYHHAHDAYLNAVVGTALIK | |
| | KYPKLESEFVYG | |
| CP1028 | EIGKATAKYFFYSNIMNFFKTEITLANGEIRKRPLIETNGETGEIVWDKGRDFAT | SEQ ID NO: 65 |
| | VRKVLSMPQVNIVKKTEVQTGGFSKESILPKRNSDKLIARKKDWDPKKYGGFDSP | |
| | TVAYSVLVVAKVEKGKSKKLKSVKELLGITIMERSSFEKNPIDFLEAKGYKEVKK | |
| | DLIIKLPKYSLFELENGRKRMLASAGELQKGNELALPSKYVNFLYLASHYEKLKG | |
| | SPEDNEQKQLFVEQHKHYLDEIIEQISEFSKRVILADANLDKVLSAYNKHRDKPI | |
| | REQAENIIHLFTLTNLGAPAAFKYFDTTIDRKRYTSTKEVLDATLIHQSITGLYE | |
| | TRIDLSQLGGDGGSGGSGGSGGSGGSGGSGGMDKKYSIGLAIGTNSVGWAVITDE | |
| | YKVPSKKFKVLGNTDRHSIKKNLIGALLFDSGETAEATRLKRTARRRYTRRKNRI | |
| | CYLQEIFSNEMAKVDDSFFHRLEESFLVEEDKKHERHPIFGNIVDEVAYHEKYPT | |
| | IYHLRKKLVDSTDKADLRLIYLALAHMIKFRGHFLIEGDLNPDNSDVDKLFIQLV | |
| | QTYNQLFEENPINASGVDAKAILSARLSKSRRLENLIAQLPGEKKNGLFGNLIAL | |
| | SLGLTPNFKSNFDLAEDAKLQLSKDTYDDDLDNLLAQIGDQYADLFLAAKNLSDA | |
| | ILLSDILRVNTEITKAPLSASMIKRYDEHHQDLTLLKALVRQQLPEKYKEIFFDQ | |
| | SKNGYAGYIDGGASQEEFYKFIKPILEKMDGTEELLVKLNREDLLRKQRTFDNGS | |
| | IPHQIHLGELHAILRRQEDFYPFLKDNREKIEKILTFRIPYYVGPLARGNSRFAW | |
| | MTRKSEETITPWNFEEVVDKGASAQSFIERMTNFDKNLPNEKVLPKHSLLYEYFT | |
| | VYNELTKVKYVTEGMRKPAFLSGEQKKAIVDLLFKTNRKVTVKQLKEDYFKKIEC | |
| | FDSVEISGVEDRFNASLGTYHDLLKIIKDKDFLDNEENEDILEDIVLTLTLFEDR | |
| | EMIEERLKTYAHLFDDKVMKQLKRRRYTGWGRLSRKLINGIRDKQSGKTILDFLK | |
| | SDGFANRNFMQLIHDDSLTFKEDIQKAQVSGQGDSLHEHIANLAGSPAIKKGILQ | |
| | TVKVVDELVKVMGRHKPENIVIEMARENQTTQKGQKNSRERMKRIEEGIKELGSQ | |
| | ILKEHPVENTQLQNEKLYLYYLQNGRDMYVDQELDINRLSDYDVDHIVPQSFLKD | |
| | DSIDNKVLTRSDKNRGKSDNVPSEEVVKKMKNYWRQLLNAKLITQRKFDNLTKAE | |
| | RGGLSELDKAGFIKRQLVETRQITKHVAQILDSRMNTKYDENDKLIREVKVITLK | |
| | SKLVSDFRKDFQFYKVREINNYHHAHDAYLNAVVGTALIKKYPKLESEFVYGDYK | |
| | VYDVRKMIAKSEQ | |
| CP1041 | NIMNFFKTEITLANGEIRKRPLIETNGETGEIVWDKGRDFATVRKVLSMPQVNIV | SEQ ID NO: 66 |
| | KKTEVQTGGFSKESILPKRNSDKLIARKKDWDPKKYGGFDSPTVAYSVLVVAKVE | |
| | KGKSKKLKSVKELLGITIMERSSFEKNPIDFLEAKGYKEVKKDLIIKLPKYSLFE | |
| | LENGRKRMLASAGELQKGNELALPSKYVNFLYLASHYEKLKGSPEDNEQKQLFVE | |
| | QHKHYLDEIIEQISEFSKRVILADANLDKVLSAYNKHRDKPIREQAENIIHLFTL | |
| | TNLGAPAAFKYFDTTIDRKRYTSTKEVLDATLIHQSITGLYETRIDLSQLGGDGG | |
| | SGGSGGSGGSGGSGGSGGDKKYSIGLAIGTNSVGWAVITDEYKVPSKKFKVLGNT | |
| | DRHSIKKNLIGALLFDSGETAEATRLKRTARRRYTRRKNRICYLQEIFSNEMAKV | |
| | DDSFFHRLEESFLVEEDKKHERHPIFGNIVDEVAYHEKYPTIYHLRKKLVDSTDK | |
| | ADLRLIYLALAHMIKFRGHFLIEGDLNPDNSDVDKLFIQLVQTYNQLFEENPINA | |
| | SGVDAKAILSARLSKSRRLENLIAQLPGEKKNGLFGNLIALSLGLTPNFKSNFDL | |
| | AEDAKLQLSKDTYDDDLDNLLAQIGDQYADLFLAAKNLSDAILLSDILRVNTEIT | |
| | KAPLSASMIKRYDEHHQDLTLLKALVRQQLPEKYKEIFFDQSKNGYAGYIDGGAS | |
| | QEEFYKFIKPILEKMDGTEELLVKLNREDLLRKQRTFDNGSIPHQIHLGELHAIL | |
| | RRQEDFYPFLKDNREKIEKILTFRIPYYVGPLARGNSRFAWMTRKSEETITPWNF | |
| | EEVVDKGASAQSFIERMTNFDKNLPNEKVLPKHSLLYEYFTVYNELTKVKYVTEG | |
| | MRKPAFLSGEQKKAIVDLLFKTNRKVTVKQLKEDYFKKIECFDSVEISGVEDRFN | |
| | ASLGTYHDLLKIIKDKDFLDNEENEDILEDIVLTLTLFEDREMIEERLKTYAHLF | |
| | DDKVMKQLKRRRYTGWGRLSRKLINGIRDKQSGKTILDFLKSDGFANRNFMQLIH | |
| | DDSLTFKEDIQKAQVSGQGDSLHEHIANLAGSPAIKKGILQTVKVVDELVKVMGR | |
| | HKPENIVIEMARENQTTQKGQKNSRERMKRIEEGIKELGSQILKEHPVENTQLQN | |
| | EKLYLYYLQNGRDMYVDQELDINRLSDYDVDHIVPQSFLKDDSIDNKVLTRSDKN | |
| | RGKSDNVPSEEVVKKMKNYWRQLLNAKLITQRKFDNLTKAERGGLSELDKAGFIK | |
| | RQLVETRQITKHVAQILDSRMNTKYDENDKLIREVKVITLKSKLVSDFRKDFQFY | |
| | KVREINNYHHAHDAYLNAVVGTALIKKYPKLESEFVYGDYKVYDVRKMIAKSEQE | |
| | IGKATAKYFFYS | |
| CP1249 | PEDNEQKQLFVEQHKHYLDEIIEQISEFSKRVILADANLDKVLSAYNKHRDKPIR | SEQ ID NO: 67 |
| | EQAENIIHLFTLTNLGAPAAFKYFDTTIDRKRYTSTKEVLDATLIHQSITGLYET | |
| | RIDLSQLGGDGGSGGSGGSGGSGGSGGSGGMDKKYSIGLAIGTNSVGWAVITDEY | |
| | KVPSKKFKVLGNTDRHSIKKNLIGALLFDSGETAEATRLKRTARRRYTRRKNRIC | |
| | YLQEIFSNEMAKVDDSFFHRLEESFLVEEDKKHERHPIFGNIVDEVAYHEKYPTI | |
| | YHLRKKLVDSTDKADLRLIYLALAHMIKFRGHFLIEGDLNPDNSDVDKLFIQLVQ | |
| | TYNQLFEENPINASGVDAKAILSARLSKSRRLENLIAQLPGEKKNGLFGNLIALS | |
| | LGLTPNFKSNFDLAEDAKLQLSKDTYDDDLDNLLAQIGDQYADLFLAAKNLSDAI | |
| | LLSDILRVNTEITKAPLSASMIKRYDEHHQDLTLLKALVRQQLPEKYKEIFFDQS | |
| | KNGYAGYIDGGASQEEFYKFIKPILEKMDGTEELLVKLNREDLLRKQRTFDNGSI | |
| | PHQIHLGELHAILRRQEDFYPFLKDNREKIEKILTFRIPYYVGPLARGNSRFAWM | |
| | TRKSEETITPWNFEEVVDKGASAQSFIERMTNFDKNLPNEKVLPKHSLLYEYFTV | |
| | YNELTKVKYVTEGMRKPAFLSGEQKKAIVDLLFKTNRKVTVKQLKEDYFKKIECF | |
| | DSVEISGVEDRFNASLGTYHDLLKIIKDKDFLDNEENEDILEDIVLTLTLFEDRE | |
| | MIEERLKTYAHLFDDKVMKQLKRRRYTGWGRLSRKLINGIRDKQSGKTILDFLKS | |
| | DGFANRNFMQLIHDDSLTFKEDIQKAQVSGQGDSLHEHIANLAGSPAIKKGILQT | |
| | VKVVDELVKVMGRHKPENIVIEMARENQTTQKGQKNSRERMKRIEEGIKELGSQI | |
| | LKEHPVENTQLQNEKLYLYYLQNGRDMYVDQELDINRLSDYDVDHIVPQSFLKDD | |
| | SIDNKVLTRSDKNRGKSDNVPSEEVVKKMKNYWRQLLNAKLITQRKFDNLTKAER | |
| | GGLSELDKAGFIKRQLVETRQITKHVAQILDSRMNTKYDENDKLIREVKVITLKS | |
| | KLVSDFRKDFQFYKVREINNYHHAHDAYLNAVVGTALIKKYPKLESEFVYGDYKV | |
| | YDVRKMIAKSEQEIGKATAKYFFYSNIMNFFKTEITLANGEIRKRPLIETNGETG | |
| | EIVWDKGRDFATVRKVLSMPQVNIVKKTEVQTGGFSKESILPKRNSDKLIARKKD | |
| | WDPKKYGGFDSPTVAYSVLVVAKVEKGKSKKLKSVKELLGITIMERSSFEKNPID | |
| | FLEAKGYKEVKKDLIIKLPKYSLFELENGRKRMLASAGELQKGNELALPSKYVNF | |
| | LYLASHYEKLKGS | |
| CP1300 | KPIREQAENIIHLFTLTNLGAPAAFKYFDTTIDRKRYTSTKEVLDATLIHQSITG | SEQ ID NO: 68 |
| | LYETRIDLSQLGGDGGSGGSGGSGGSGGSGGSGGDKKYSIGLAIGTNSVGWAVIT | |
| | DEYKVPSKKFKVLGNTDRHSIKKNLIGALLFDSGETAEATRLKRTARRRYTRRKN | |
| | RICYLQEIFSNEMAKVDDSFFHRLEESFLVEEDKKHERHPIFGNIVDEVAYHEKY | |
| | PTIYHLRKKLVDSTDKADLRLIYLALAHMIKFRGHFLIEGDLNPDNSDVDKLFIQ | |
| | LVQTYNQLFEENPINASGVDAKAILSARLSKSRRLENLIAQLPGEKKNGLFGNLI | |
| | ALSLGLTPNFKSNFDLAEDAKLQLSKDTYDDDLDNLLAQIGDQYADLFLAAKNLS | |
| | DAILLSDILRVNTEITKAPLSASMIKRYDEHHQDLTLLKALVRQQLPEKYKEIFF | |
| | DQSKNGYAGYIDGGASQEEFYKFIKPILEKMDGTEELLVKLNREDLLRKQRTFDN | |
| | GSIPHQIHLGELHAILRRQEDFYPFLKDNREKIEKILTFRIPYYVGPLARGNSRF | |
| | AWMTRKSEETITPWNFEEVVDKGASAQSFIERMTNFDKNLPNEKVLPKHSLLYEY | |
| | FTVYNELTKVKYVTEGMRKPAFLSGEQKKAIVDLLFKTNRKVTVKQLKEDYFKKI | |
| | ECFDSVEISGVEDRFNASLGTYHDLLKIIKDKDFLDNEENEDILEDIVLTLTLFE | |
| | DREMIEERLKTYAHLFDDKVMKQLKRRRYTGWGRLSRKLINGIRDKQSGKTILDF | |
| | LKSDGFANRNFMQLIHDDSLTFKEDIQKAQVSGQGDSLHEHIANLAGSPAIKKGI | |
| | LQTVKVVDELVKVMGRHKPENIVIEMARENQTTQKGQKNSRERMKRIEEGIKELG | |
| | SQILKEHPVENTQLQNEKLYLYYLQNGRDMYVDQELDINRLSDYDVDHIVPQSFL | |
| | KDDSIDNKVLTRSDKNRGKSDNVPSEEVVKKMKNYWRQLLNAKLITQRKFDNLTK | |
| | AERGGLSELDKAGFIKRQLVETRQITKHVAQILDSRMNTKYDENDKLIREVKVIT | |
| | LKSKLVSDFRKDFQFYKVREINNYHHAHDAYLNAVVGTALIKKYPKLESEFVYGD | |
| | YKVYDVRKMIAKSEQEIGKATAKYFFYSNIMNFFKTEITLANGEIRKRPLIETNG | |
| | ETGEIVWDKGRDFATVRKVLSMPQVNIVKKTEVQTGGFSKESILPKRNSDKLIAR | |
| | KKDWDPKKYGGFDSPTVAYSVLVVAKVEKGKSKKLKSVKELLGITIMERSSFEKN | |
| | PIDFLEAKGYKEVKKDLIIKLPKYSLFELENGRKRMLASAGELQKGNELALPSKY | |
| | VNFLYLASHYEKLKGSPEDNEQKQLFVEQHKHYLDEIIEQISEFSKRVILADANL | |
| | DKVLSAYNKHRD | |

The Cas9 circular permutants that may be useful in the base editing constructs described herein. Exemplary C-terminal fragments of Cas9, based on the Cas9 of SEQ TD NO: 5, which may be rearranged to an N-terminus of Cas9, are provided below. It should be appreciated that such C-terminal fragments of Cas9 are exemplary and are not meant to be limiting. These exemplary CP-Cas9 fragments have the following sequences:

| CP name | Sequence | SEQ ID NO: |
|---|---|---|
| CP1012 c- | DYKVYDVRKMIAKSEQEIGKATAKYFFYSNIMNFFKTEITLANGEIRKRPLIETN | 69 |
| terminal | GETGEIVWDKGRDFATVRKVLSMPQVNIVKKTEVQTGGFSKESILPKRNSDKLIA | |
| fragment | RKKDWDPKKYGGFDSPTVAYSVLVVAKVEKGKSKKLKSVKELLGITIMERSSFEK | |
| | NPIDFLEAKGYKEVKKDLIIKLPKYSLFELENGRKRMLASAGELQKGNELALPSK | |
| | YVNFLYLASHYEKLKGSPEDNEQKQLFVEQHKHYLDEIIEQISEFSKRVILADAN | |
| | LDKVLSAYNKHRDKPIREQAENIIHLFTLTNLGAPAAFKYFDTTIDRKRYTSTKE | |
| | VLDATLIHQSITGLYETRIDLSQLGGD | |
| CP1028 c- | EIGKATAKYFFYSNIMNFFKTEITLANGEIRKRPLIETNGETGEIVWDKGRDFAT | 70 |
| terminal | VRKVLSMPQVNIVKKTEVQTGGFSKESILPKRNSDKLIARKKDWDPKKYGGFDSP | |
| fragment | TVAYSVLVVAKVEKGKSKKLKSVKELLGITIMERSSFEKNPIDFLEAKGYKEVKK | |
| | DLIIKLPKYSLFELENGRKRMLASAGELQKGNELALPSKYVNFLYLASHYEKLKG | |
| | SPEDNEQKQLFVEQHKHYLDEIIEQISEFSKRVILADANLDKVLSAYNKHRDKPI | |
| | REQAENIIHLFTLTNLGAPAAFKYFDTTIDRKRYTSTKEVLDATLIHQSITGLYE | |
| | TRIDLSQLGGD | |
| CP1041 c- | NIMNFFKTEITLANGEIRKRPLIETNGETGEIVWDKGRDFATVRKVLSMPQVNIV | 71 |
| terminal | KKTEVQTGGFSKESILPKRNSDKLIARKKDWDPKKYGGFDSPTVAYSVLVVAKVE | |
| fragment | KGKSKKLKSVKELLGITIMERSSFEKNPIDFLEAKGYKEVKKDLIIKLPKYSLFE | |
| | LENGRKRMLASAGELQKGNELALPSKYVNFLYLASHYEKLKGSPEDNEQKQLFVE | |
| | QHKHYLDEIIEQISEFSKRVILADANLDKVLSAYNKHRDKPIREQAENIIHLFTL | |
| | TNLGAPAAFKYFDTTIDRKRYTSTKEVLDATLIHQSITGLYETRIDLSQLGGD | |
| CP1249 c- | PEDNEQKQLFVEQHKHYLDEIIEQISEFSKRVILADANLDKVLSAYNKHRDKPIR | 72 |
| terminal | EQAENIIHLFTLTNLGAPAAFKYFDTTIDRKRYTSTKEVLDATLIHQSITGLYET | |
| fragment | RIDLSQLGGD | |
| CP1300 c- | KPIREQAENIIHLFTLTNLGAPAAFKYFDTTIDRKRYTSTKEVLDATLIHQSITG | 73 |
| terminal | LYETRIDLSQLGGD | |
| fragment | | |

(10) Cas9 Variants with Modified PAM Specificities

The base editors of the present disclosure may also comprise Cas9 variants with modified PAM specificities. For example, the base editors described herein may utilize any naturally occurring or engineered variant of SpCas9 having expanded and/or relaxed PAM specificities which are described in the literature, including in Nishimasu et al., “Engineered CRISPR-Cas9 nuclease with expanded targeting space,” Science, 2018, 361: 1259-1262; Chatterjee et al., “Robust Genome Editing of Single-Base PAM Targets with Engineered ScCas9 Variants,” BioRxiv, Apr. 26, 2019 Some aspects of this disclosure provide Cas9 proteins that exhibit activity on a target sequence that does not comprise the canonical PAM (5′-NGG-3′, where N is A, C, G, or T) at its 3′-end. In some embodiments, the Cas9 protein exhibits activity on a target sequence comprising a 5′-NGG-3′ PAM sequence at its 3′-end. In some embodiments, the Cas9 protein exhibits activity on a target sequence comprising a 5′-NNG-3′ PAM sequence at its 3′-end. In some embodiments, the Cas9 protein exhibits activity on a target sequence comprising a 5′-NNA-3′ PAM sequence at its 3′-end. In some embodiments, the Cas9 protein exhibits activity on a target sequence comprising a 5′-NNC-3′ PAM sequence at its 3′-end. In some embodiments, the Cas9 protein exhibits activity on a target sequence comprising a 5′-NNT-3′ PAM sequence at its 3′-end. In some embodiments, the Cas9 protein exhibits activity on a target sequence comprising a 5′-NGT-3′ PAM sequence at its 3′-end. In some embodiments, the Cas9 protein exhibits activity on a target sequence comprising a 5′-NGA-3′ PAM sequence at its 3′-end. In some embodiments, the Cas9 protein exhibits activity on a target sequence comprising a 5′-NGC-3′ PAM sequence at its 3′-end. In some embodiments, the Cas9 protein exhibits activity on a target sequence comprising a 5′-NAA-3′ PAM sequence at its 3′-end. In some embodiments, the Cas9 protein exhibits activity on a target sequence comprising a 5′-NAC-3′ PAM sequence at its 3′-end. In some embodiments, the Cas9 protein exhibits activity on a target sequence comprising a 5′-NAT-3′ PAM sequence at its 3′-end. In still other embodiments, the Cas9 protein exhibits activity on a target sequence comprising a 5′-NAG-3′ PAM sequence at its 3′-end.

It should be appreciated that any of the amino acid mutations described herein, (e.g., A262T) from a first amino acid residue (e.g., A) to a second amino acid residue (e.g., T) may also include mutations from the first amino acid residue to an amino acid residue that is similar to (e.g., conserved) the second amino acid residue. For example, mutation of an amino acid with a hydrophobic side chain (e.g., alanine, valine, isoleucine, leucine, methionine, phenylalanine, tyrosine, or tryptophan) may be a mutation to a second amino acid with a different hydrophobic side chain (e.g., alanine, valine, isoleucine, leucine, methionine, phenylalanine, tyrosine, or tryptophan). For example, a mutation of an alanine to a threonine (e.g., a A262T mutation) may also be a mutation from an alanine to an amino acid that is similar in size and chemical properties to a threonine, for example, serine. As another example, mutation of an amino acid with a positively charged side chain (e.g., arginine, histidine, or lysine) may be a mutation to a second amino acid with a different positively charged side chain (e.g., arginine, histidine, or lysine). As another example, mutation of an amino acid with a polar side chain (e.g., serine, threonine, asparagine, or glutamine) may be a mutation to a second amino acid with a different polar side chain (e.g., serine, threonine, asparagine, or glutamine). Additional similar amino acid pairs include, but are not limited to, the following: phenylalanine and tyrosine; asparagine and glutamine; methionine and cysteine; aspartic acid and glutamic acid; and arginine and lysine. The skilled artisan would recognize that such conservative amino acid substitutions will likely have minor effects on protein structure and are likely to be well tolerated without compromising function. In some embodiments, any amino of the amino acid mutations provided herein from one amino acid to a threonine may be an amino acid mutation to a serine. In some embodiments, any amino of the amino acid mutations provided herein from one amino acid to an arginine may be an amino acid mutation to a lysine. In some embodiments, any amino of the amino acid mutations provided herein from one amino acid to an isoleucine, may be an amino acid mutation to an alanine, valine, methionine, or leucine. In some embodiments, any amino of the amino acid mutations provided herein from one amino acid to a lysine may be an amino acid mutation to an arginine. In some embodiments, any amino of the amino acid mutations provided herein from one amino acid to an aspartic acid may be an amino acid mutation to a glutamic acid or asparagine. In some embodiments, any amino of the amino acid mutations provided herein from one amino acid to a valine may be an amino acid mutation to an alanine, isoleucine, methionine, or leucine. In some embodiments, any amino of the amino acid mutations provided herein from one amino acid to a glycine may be an amino acid mutation to an alanine. It should be appreciated, however, that additional conserved amino acid residues would be recognized by the skilled artisan and any of the amino acid mutations to other conserved amino acid residues are also within the scope of this disclosure.

In some embodiments, the present disclosure may utilize any of the Cas9 variants disclosed in the SEQUENCES section herein.

In some embodiments, the Cas9 protein comprises a combination of mutations that exhibit activity on a target sequence comprising a 5′-NAA-3′ PAM sequence at its 3′-end. In some embodiments, the combination of mutations are present in any one of the clones listed in Table 1. In some embodiments, the combination of mutations are conservative mutations of the clones listed in Table 1. In some embodiments, the Cas9 protein comprises the combination of mutations of any one of the Cas9 clones listed in Table 1.

TABLE 1
NAA PAM Clones
Mutations from wild-type SpCas9 (e.g., SEQ ID NO: 5)

D177N, K218R, D614N, D1135N, P1137S, E1219V, A1320V, A1323D, R1333K
D177N, K218R, D614N, D1135N, E1219V, Q1221H, H1264Y, A1320V, R1333K
A10T, I322V, S409I, E427G, G715C, D1135N, E1219V, Q1221H, H1264Y, A1320V, R1333K
A367T, K710E, R1114G, D1135N, P1137S, E1219V, Q1221H, H1264Y, A1320V, R1333K
A10T, I322V, S409I, E427G, R753G, D861N, D1135N, K1188R, E1219V, Q1221H, H1264H,
A1320V, R1333K
A10T, I322V, S409I, E427G, R654L, V743I, R753G, M1021T, D1135N, D1180G, K1211R,
E1219V, Q1221H, H1264Y, A1320V, R1333K
A10T, I322V, S409I, E427G, V743I, R753G, E762G, D1135N, D1180G, K1211R, E1219V,
Q1221H, H1264Y, A1320V, R1333K
A10T, I322V, S409I, E427G, R753G, D1135N, D1180G, K1211R, E1219V, Q1221H, H1264Y,
S1274R, A1320V, R1333K
A10T, I322V, S409I, E427G, A589S, R753G, D1135N, E1219V, Q1221H, H1264H, A1320V,
R1333K
A10T, I322V, S409I, E427G, R753G, E757K, G865G, D1135N, E1219V, Q1221H, H1264Y,
A1320V, R1333K
A10T, I322V, S409I, E427G, R654L, R753G, E757K, D1135N, E1219V, Q1221H, H1264Y,
A1320V, R1333K
A10T, I322V, S409I, E427G, K599R, M631A, R654L, K673E, V743I, R753G, N758H, E762G,
D1135N, D1180G, E1219V, Q1221H, Q1256R, H1264Y, A1320V, A1323D, R1333K
A10T, I322V, S409I, E427G, R654L, K673E, V743I, R753G, E762G, N869S, N1054D, R1114G,
D1135N, D1180G, E1219V, Q1221H, H1264Y, A1320V, A1323D, R1333K
A10T, I322V, S409I, E427G, R654L, L727I, V743I, R753G, E762G, R859S, N946D, F1134L,
D1135N, D1180G, E1219V, Q1221H, H1264Y, N1317T, A1320V, A1323D, R1333K
A10T, I322V, S409I, E427G, R654L, K673E, V743I, R753G, E762G, N803S, N869S, Y1016D,
G1077D, R1114G, F1134L, D1135N, D1180G, E1219V, Q1221H, H1264Y, V1290G, L1318S,
A1320V, A1323D, R1333K
A10T, I322V, S409I, E427G, R654L, K673E, V743I, R753G, E762G, N803S, N869S, Y1016D,
G1077D, R1114G, F1134L, D1135N, K1151E, D1180G, E1219V, Q1221H, H1264Y, V1290G,
L1318S, A1320V, R1333K
A10T, I322V, S409I, E427G, R654L, K673E, V743I, R753G, E762G, N803S, N869S, Y1016D,
G1077D, R1114G, F1134L, D1135N, D1180G, E1219V, Q1221H, H1264Y, V1290G, L1318S,
A1320V, A1323D, R1333K
A10T, I322V, S409I, E427G, R654L, K673E, F693L, V743I, R753G, E762G, N803S, N869S,
L921P, Y1016D, G1077D, F1080S, R1114G, D1135N, D1180G, E1219V, Q1221H, H1264Y,
L1318S, A1320V, A1323D, R1333K
A10T, I322V, S409I, E427G, E630K, R654L, K673E, V743I, R753G, E762G, Q768H, N803S,
N869S, Y1016D, G1077D, R1114G, F1134L, D1135N, D1180G, E1219V, Q1221H, H1264Y,
L1318S, A1320V, R1333K
A10T, I322V, S409I, E427G, R654L, K673E, F693L, V743I, R753G, E762G, Q768H, N803S,
N869S, Y1016D, G1077D, R1114G, F1134L, D1135N, D1180G, E1219V, Q1221H, G1223S,
H1264Y, L1318S, A1320V, R1333K
A10T, I322V, S409I, E427G, R654L, K673E, F693L, V743I, R753G, E762G, N803S, N869S,
L921P, Y1016D, G1077D, F1801S, R1114G, D1135N, D1180G, E1219V, Q1221H, H1264Y,
L1318S, A1320V, A1323D, R1333K
A10T, I322V, S409I, E427G, R654L, V743I, R753G, M1021T, D1135N, D1180G, K1211R,
E1219V, Q1221H, H1264Y, A1320V, R1333K
A10T, I322V, S409I, E427G, R654L, K673E, V743I, R753G, E762G, M673I, N803S, N869S,
G1077D, R1114G, D1135N, V1139A, D1180G, E1219V, Q1221H, A1320V, R1333K
A10T, I322V, S409I, E427G, R654L, K673E, V743I, R753G, E762G, N803S, N869S, R1114G,
D1135N, E1219V, Q1221H, A1320V, R1333K

In some embodiments, the Cas9 protein comprises an amino acid sequence that is at least 80% identical to the amino acid sequence of a Cas9 protein as provided by any one of the variants of Table 1. In some embodiments, the Cas9 protein comprises an amino acid sequence that is at least 85%, at least 90%, at least 92%, at least 95%, at least 96%, at least 97%, at least 98%, at least 99%, or at least 99.5% identical to the amino acid sequence of a Cas9 protein as provided by any one of the variants of Table 1.

In some embodiments, the Cas9 protein exhibits an increased activity on a target sequence that does not comprise the canonical PAM (5′-NGG-3′) at its 3′ end as compared to Streptococcus pyogenes Cas9 as provided by SEQ ID NO: 5. In some embodiments, the Cas9 protein exhibits an activity on a target sequence having a 3′ end that is not directly adjacent to the canonical PAM sequence (5′-NGG-3′) that is at least 5-fold increased as compared to the activity of Streptococcus pyogenes Cas9 as provided by SEQ ID NO: 5 on the same target sequence. In some embodiments, the Cas9 protein exhibits an activity on a target sequence that is not directly adjacent to the canonical PAM sequence (5′-NGG-3′) that is at least 10-fold, at least 50-fold, at least 100-fold, at least 500-fold, at least 1,000-fold, at least 5,000-fold, at least 10,000-fold, at least 50,000-fold, at least 100,000-fold, at least 500,000-fold, or at least 1,000,000-fold increased as compared to the activity of Streptococcus pyogenes as provided by SEQ ID NO: 5 on the same target sequence. In some embodiments, the 3′ end of the target sequence is directly adjacent to an AAA, GAA, CAA, or TAA sequence. In some embodiments, the Cas9 protein comprises a combination of mutations that exhibit activity on a target sequence comprising a 5′-NAC-3′ PAM sequence at its 3′-end. In some embodiments, the combination of mutations are present in any one of the clones listed in Table 2. In some embodiments, the combination of mutations are conservative mutations of the clones listed in Table 2. In some embodiments, the Cas9 protein comprises the combination of mutations of any one of the Cas9 clones listed in Table 2.

TABLE 2
NAC PAM Clones
MUTATIONS FROM WILD-TYPE SPCAS9 (E.G., SEQ ID NO: 5)

T472I, R753G, K890E, D1332N, R1335Q, T1337N
I1057S, D1135N, P1301S, R1335Q, T1337N
T472I, R753G, D1332N, R1335Q, T1337N
D1135N, E1219V, D1332N, R1335Q, T1337N
T472I, R753G, K890E, D1332N, R1335Q, T1337N
I1057S, D1135N, P1301S, R1335Q, T1337N
T472I, R753G, D1332N, R1335Q, T1337N
T472I, R753G, Q771H, D1332N, R1335Q, T1337N
E627K, T638P, K652T, R753G, N803S, K959N, R1114G, D1135N, E1219V, D1332N, R1335Q,
T1337N
E627K, T638P, K652T, R753G, N803S, K959N, R1114G, D1135N, K1156E, E1219V, D1332N,
R1335Q, T1337N
E627K, T638P, V647I, R753G, N803S, K959N, G1030R, I1055E, R1114G, D1135N, E1219V,
D1332N, R1335Q, T1337N
E627K, E630G, T638P, V647A, G687R, N767D, N803S, K959N, R1114G, D1135N, E1219V,
D1332G, R1335Q, T1337N
E627K, T638P, R753G, N803S, K959N, R1114G, D1135N, E1219V, N1266H, D1332N, R1335Q,
T1337N
E627K, T638P, R753G, N803S, K959N, I1057T, R1114G, D1135N, E1219V, D1332N, R1335Q,
T1337N
E627K, T638P, R753G, N803S, K959N, R1114G, D1135N, E1219V, D1332N, R1335Q, T1337N
E627K, M631I, T638P, R753G, N803S, K959N, Y1036H, R1114G, D1135N, E1219V, D1251G,
D1332G, R1335Q, T1337N
E627K, T638P, R753G, N803S, V875I, K959N, Y1016C, R1114G, D1135N, E1219V, D1251G,
D1332G, R1335Q, T1337N, I1348V
K608R, E627K, T638P, V647I, R654L, R753G, N803S, T804A, K848N, V922A, K959N, R1114G,
D1135N, E1219V, D1332N, R1335Q, T1337N
K608R, E627K, T638P, V647I, R753G, N803S, V922A, K959N, K1014N, V1015A, R1114G,
D1135N, K1156N, E1219V, N1252D, D1332N, R1335Q, T1337N
K608R, E627K, R629G, T638P, V647I, A711T, R753G, K775R, K789E, N803S, K959N, V1015A,
Y1036H, R1114G, D1135N, E1219V, N1286H, D1332N, R1335Q, T1337N
K608R, E627K, T638P, V647I, T740A, R753G, N803S, K948E, K959N, Y1016S, R1114G,
D1135N, E1219V, N1286H, D1332N, R1335Q, T1337N
K608R, E627K, T638P, V647I, T740A, N803S, K948E, K959N, Y1016S, R1114G, D1135N,
E1219V, N1286H, D1332N, R1335Q, T1337N
I670S, K608R, E627K, E630G, T638P, V647I, R653K, R753G, I795L, K797N, N803S, K866R,
K890N, K959N, Y1016C, R1114G, D1135N, E1219V, D1332N, R1335Q, T1337N
K608R, E627K, T638P, V647I, T740A, G752R, R753G, K797N, N803S, K948E, K959N, V1015A,
Y1016S, R1114G, D1135N, E1219V, N1266H, D1332N, R1335Q, T1337N
I570T, A589V, K608R, E627K, T638P, V647I, R654L, Q716R, R753G, N803S, K948E, K959N,
Y1016S, R1114G, D1135N, E1207G, E1219V, N1234D, D1332N, R1335Q, T1337N
K608R, E627K, R629G, T638P, V647I, R654L, Q740R, R753G, N803S, K959N, N990S, T995S,
V1015A, Y1036D, R1114G, D1135N, E1207G, E1219V, N1234D, N1266H, D1332N, R1335Q,
T1337N
I562F, V565D, I570T, K608R, L625S, E627K, T638P, V647I, R654I, G752R, R753G, N803S,
N808D, K959N, M1021L, R1114G, D1135N, N1177S, N1234D, D1332N, R1335Q, T1337N
I562F, I570T, K608R, E627K, T638P, V647I, R753G, E790A, N803S, K959N, V1015A, Y1036H,
R1114G, D1135N, D1180E, A1184T, E1219V, D1332N, R1335Q, T1337N
I570T, K608R, E627K, T638P, V647I, R654H, R753G, E790A, N803S, K959N, V1015A, R1114G,
D1127A, D1135N, E1219V, D1332N, R1335Q, T1337N
I570T, K608R, L625S, E627K, T638P, V647I, R654I, T703P, R753G, N803S, N808D, K959N,
M1021L, R1114G, D1135N, E1219V, D1332N, R1335Q, T1337N
I570S, K608R, E627K, E630G, T638P, V647I, R653K, R753G, I795L, N803S, K866R, K890N,
K959N, Y1016C, R1114G, D1135N, E1219V, D1332N, R1335Q, T1337N
I570T, K608R, E627K, T638P, V647I, R654H, R753G, E790A, N803S, K959N, V1016A, R1114G,
D1135N, E1219V, K1246E, D1332N, R1335Q, T1337N
K608R, E627K, T638P, V647I, R654L, K673E, R753G, E790A, N803S, K948E, K959N, R1114G,
D1127G, D1135N, D1180E, E1219V, N1286H, D1332N, R1335Q, T1337N
K608R, L625S, E627K, T638P, V647I, R654I, I670T, R753G, N803S, N808D, K959N, M1021L,
R1114G, D1135N, E1219V, N1286H, D1332N, R1335Q, T1337N
E627K, M631V, T638P, V647I, K710E, R753G, N803S, N808D, K948E, M1021L, R1114G,
D1135N, E1219V, D1332N, R1335Q, T1337N, S1338T, H1349R

In some embodiments, the Cas9 protein comprises an amino acid sequence that is at least 80% identical to the amino acid sequence of a Cas9 protein as provided by any one of the variants of Table 2. In some embodiments, the Cas9 protein comprises an amino acid sequence that is at least 85%, at least 90%, at least 92%, at least 95%, at least 96%, at least 97%, at least 98%, at least 99%, or at least 99.5% identical to the amino acid sequence of a Cas9 protein as provided by any one of the variants of Table 2.

In some embodiments, the Cas9 protein exhibits an increased activity on a target sequence that does not comprise the canonical PAM (5′-NGG-3′) at its 3′ end as compared to Streptococcus pyogenes Cas9 as provided by SEQ ID NO: 5. In some embodiments, the Cas9 protein exhibits an activity on a target sequence having a 3′ end that is not directly adjacent to the canonical PAM sequence (5′-NGG-3′) that is at least 5-fold increased as compared to the activity of Streptococcus pyogenes Cas9 as provided by SEQ ID NO: 5 on the same target sequence. In some embodiments, the Cas9 protein exhibits an activity on a target sequence that is not directly adjacent to the canonical PAM sequence (5′-NGG-3′) that is at least 10-fold, at least 50-fold, at least 100-fold, at least 500-fold, at least 1,000-fold, at least 5,000-fold, at least 10,000-fold, at least 50,000-fold, at least 100,000-fold, at least 500,000-fold, or at least 1,000,000-fold increased as compared to the activity of Streptococcus pyogenes as provided by SEQ ID NO: 5 on the same target sequence. In some embodiments, the 3′ end of the target sequence is directly adjacent to an AAC, GAC, CAC, or TAC sequence.

In some embodiments, the Cas9 protein comprises a combination of mutations that exhibit activity on a target sequence comprising a 5′-NAT-3′ PAM sequence at its 3′-end. In some embodiments, the combination of mutations are present in any one of the clones listed in Table 3. In some embodiments, the combination of mutations are conservative mutations of the clones listed in Table 3. In some embodiments, the Cas9 protein comprises the combination of mutations of any one of the Cas9 clones listed in Table 3.

TABLE 3
NAT PAM Clones
MUTATIONS FROM WILD-TYPE SPCAS9 (E.G., SEQ ID NO: 5)

K961E, H985Y, D1135N, K1191N, E1219V, Q1221H, A1320A, P1321S, R1335L
D1135N, G1218S, E1219V, Q1221H, P1249S, P1321S, D1322G, R1335L
V743I, R753G, E790A, D1135N, G1218S, E1219V, Q1221H, A1227V, P1249S, N1286K, A1293T,
P1321S, D1322G, R1335L, T1339I
F575S, M631L, R654L, V748I, V743I, R753G, D853E, V922A, R1114G D1135N, G1218S,
E1219V, Q1221H, A1227V, P1249S, N1286K, A1293T, P1321S, D1322G, R1335L, T1339I
F575S, M631L, R654L, R664K, R753G, D853E, V922A, R1114G D1135N, D1180G, G1218S,
E1219V, Q1221H, P1249S, N1286K, P1321S, D1322G, R1335L
M631L, R654L, R753G, K797E, D853E, V922A, D1012A, R1114G D1135N, G1218S, E1219V,
Q1221H, P1249S, N1317K, P1321S, D1322G, R1335L
F575S, M631L, R654L, R664K, R753G, D853E, V922A, R1114G, Y1131C, D1135N, D1180G,
G1218S, E1219V, Q1221H, P1249S, P1321S, D1322G, R1335L
F575S, M631L, R654L, R664K, R753G, D853E, V922A, R1114G, Y1131C, D1135N, D1180G,
G1218S, E1219V, Q1221H, P1249S, P1321S, D1322G, R1335L
F575S, D596Y, M631L, R654L, R664K, R753G, D853E, V922A, R1114G, Y1131C, D1135N,
D1180G, G1218S, E1219V, Q1221H, P1249S, Q1256R, P1321S, D1322G, R1335L
F575S, M631L, R654L, R664K, K710E, V750A, R753G, D853E, V922A, R1114G, Y1131C,
D1135N, D1180G, G1218S, E1219V, Q1221H, P1249S, P1321S, D1322G, R1335L
F575S, M631L, K649R, R654L, R664K, R753G, D853E, V922A, R1114G, Y1131C, D1135N,
K1156E, D1180G, G1218S, E1219V, Q1221H, P1249S, P1321S, D1322G, R1335L
F575S, M631L, R654L, R664K, R753G, D853E, V922A, R1114G, Y1131C, D1135N, D1180G,
G1218S, E1219V, Q1221H, P1249S, P1321S, D1322G, R1335L
F575S, M631L, R654L, R664K, R753G, D853E, V922A, I1057G, R1114G, Y1131C, D1135N,
D1180G, G1218S, E1219V, Q1221H, P1249S, N1308D, P1321S, D1322G, R1335L
M631L, R654L, R753G, D853E, V922A, R1114G, Y1131C, D1135N, E1150V, D1180G, G1218S,
E1219V, Q1221H, P1249S, P1321S, D1332G, R1335L
M631L, R654L, R664K, R753G, D853E, I1057V, Y1131C, D1135N, D1180G, G1218S, E1219V,
Q1221H, P1249S, P1321S, D1332G, R1335L
M631L, R654L, R664K, R753G, I1057V, R1114G, Y1131C, D1135N, D1180G, G1218S, E1219V,
Q1221H, P1249S, P1321S, D1332G, R1335L

(i) The above description of various napDNAbps which can be used in connection with the presently disclose base editors is not meant to be limiting in any way. The base editors may comprise the canonical SpCas9, or any ortholog Cas9 protein, or any variant Cas9 protein—including any naturally occurring variant, mutant, or otherwise engineered version of Cas9—that is known or which can be made or evolved through a directed evolutionary or otherwise mutagenic process. In various embodiments, the Cas9 or Cas9 variants have a nickase activity, i.e., only cleave of strand of the target DNA sequence. In other embodiments, the Cas9 or Cas9 variants have inactive nucleases, i.e., are “dead” Cas9 proteins. Other variant Cas9 proteins that may be used are those having a smaller molecular weight than the canonical SpCas9 (e.g., for easier delivery) or having modified or rearranged primary amino acid structure (e.g., the circular permutant formats). The base editors described herein may also comprise Cas9 equivalents, including Cas12a/Cpf1 and Cas12b proteins which are the result of convergent evolution. The napDNAbps used herein (e.g., SpCas9, Cas9 variant, or Cas9 equivalents) may also may also contain various modifications that alter/enhance their PAM specifities. Lastly, the application contemplates any Cas9, Cas9 variant, or Cas9 equivalent which has at least 70%, at least 75%, at least 80%, at least 85%, at least 90%, at least 91%, at least 92%, at least 93%, at least 94%, at least 95%, at least 96%, at least 97%, at least 98%, at least 99%, or at least 99.9% sequence identity to a reference Cas9 sequence, such as a references SpCas9 canonical sequences or a reference Cas9 equivalent (e.g., Cas12a/Cpf1).

In a particular embodiment, the Cas9 variant having expanded PAM capabilities is SpCas9 (H840A) VRQR, having the following amino acid sequence (with the V, R, Q, R substitutions relative to the SpCas9 (H840A) of SEQ ID NO: 42 show in bold underline. In addition, the methionine residue in SpCas9 (H840) was removed for SpCas9 (H840A) VRQR) (“SpCas9-VRQR”). This SpCas9 variant possesses an altered PAM-specificity which recognizes a PAM of 5′-NGA-3′ instead of the canonical PAM of 5′-NGG-3′:

SpCas9-VRQR
(SEQ ID NO: 74)
DKKYSIGLDIGTNSVGWAVITDEYKVPSKKFKVLGNTDRHSIKKNLIGALLFDSGETAEATRLKRTARRRYTRRKNRICYL
QEIFSNEMAKVDDSFFHRLEESFLVEEDKKHERHPIFGNIVDEVAYHEKYPTIYHLRKKLVDSTDKADLRLIYLALAHMIK
FRGHFLIEGDLNPDNSDVDKLFIQLVQTYNQLFEENPINASGVDAKAILSARLSKSRRLENLIAQLPGEKKNGLFGNLIAL
SLGLTPNFKSNFDLAEDAKLQLSKDTYDDDLDNLLAQIGDQYADLFLAAKNLSDAILLSDILRVNTEITKAPLSASMIKRY
DEHHQDLTLLKALVRQQLPEKYKEIFFDQSKNGYAGYIDGGASQEEFYKFIKPILEKMDGTEELLVKLNREDLLRKQRTFD
NGSIPHQIHLGELHAILRRQEDFYPFLKDNREKIEKILTFRIPYYVGPLARGNSRFAWMTRKSEETITPWNFEEVVDKGAS
AQSFIERMTNFDKNLPNEKVLPKHSLLYEYFTVYNELTKVKYVTEGMRKPAFLSGEQKKAIVDLLFKTNRKVTVKQLKEDY
FKKIECFDSVEISGVEDRFNASLGTYHDLLKIIKDKDFLDNEENEDILEDIVLTLTLFEDREMIEERLKTYAHLFDDKVMK
QLKRRRYTGWGRLSRKLINGIRDKQSGKTILDFLKSDGFANRNFMQLIHDDSLTFKEDIQKAQVSGQGDSLHEHIANLAGS
PAIKKGILQTVKVVDELVKVMGRHKPENIVIEMARENQTTQKGQKNSRERMKRIEEGIKELGSQILKEHPVENTQLQNEKL
YLYYLQNGRDMYVDQELDINRLSDYDVDAIVPQSFLKDDSIDNKVLTRSDKNRGKSDNVPSEEVVKKMKNYWRQLLNAKLI
TQRKFDNLTKAERGGLSELDKAGFIKRQLVETRQITKHVAQILDSRMNTKYDENDKLIREVKVITLKSKLVSDFRKDFQFY
KVREINNYHHAHDAYLNAVVGTALIKKYPKLESEFVYGDYKVYDVRKMIAKSEQEIGKATAKYFFYSNIMNFFKTEITLAN
GEIRKRPLIETNGETGEIVWDKGRDFATVRKVLSMPQVNIVKKTEVQTGGFSKESILPKRNSDKLIARKKDWDPKKYGGFV
SPTVAYSVLVVAKVEKGKSKKLKSVKELLGITIMERSSFEKNPIDFLEAKGYKEVKKDLIIKLPKYSLFELENGRKRMLAS
ARELQKGNELALPSKYVNFLYLASHYEKLKGSPEDNEQKQLFVEQHKHYLDEIIEQISEFSKRVILADANLDKVLSAYNKH
RDKPIREQAENIIHLFTLTNLGAPAAFKYFDTTIDRKQYRSTKEVLDATLIHQSITGLYETRIDLSQLGGD

In another particular embodiment, the Cas9 variant having expanded PAM capabilities is SpCas9 (H840A) VQR, having the following amino acid sequence (with the V, Q, R substitutions relative to the SpCas9 (H840A) of SEQ ID NO: 42 show in bold underline. In addition, the methionine residue in SpCas9 (H840) was removed for SpCas9 (H840A) VRQR) (“SpCas9-VQR”). This SpCas9 variant possesses an altered PAM-specificity which recognizes a PAM of 5′-NGA-3′ instead of the canonical PAM of 5′-NGG-3′:

SpCas9-VQR
(SEQ ID NO: 75)
DKKYSIGLDIGTNSVGWAVITDEYKVPSKKFKVLGNTDRHSIKKNLIGALLFDSGETAEATRLKRTARRRYTRRKNRICYL
QEIFSNEMAKVDDSFFHRLEESFLVEEDKKHERHPIFGNIVDEVAYHEKYPTIYHLRKKLVDSTDKADLRLIYLALAHMIK
FRGHFLIEGDLNPDNSDVDKLFIQLVQTYNQLFEENPINASGVDAKAILSARLSKSRRLENLIAQLPGEKKNGLFGNLIAL
SLGLTPNFKSNFDLAEDAKLQLSKDTYDDDLDNLLAQIGDQYADLFLAAKNLSDAILLSDILRVNTEITKAPLSASMIKRY
DEHHQDLTLLKALVRQQLPEKYKEIFFDQSKNGYAGYIDGGASQEEFYKFIKPILEKMDGTEELLVKLNREDLLRKQRTFD
NGSIPHQIHLGELHAILRRQEDFYPFLKDNREKIEKILTFRIPYYVGPLARGNSRFAWMTRKSEETITPWNFEEVVDKGAS
AQSFIERMTNFDKNLPNEKVLPKHSLLYEYFTVYNELTKVKYVTEGMRKPAFLSGEQKKAIVDLLFKTNRKVTVKQLKEDY
FKKIECFDSVEISGVEDRFNASLGTYHDLLKIIKDKDFLDNEENEDILEDIVLTLTLFEDREMIEERLKTYAHLFDDKVMK
QLKRRRYTGWGRLSRKLINGIRDKQSGKTILDFLKSDGFANRNFMQLIHDDSLTFKEDIQKAQVSGQGDSLHEHIANLAGS
PAIKKGILQTVKVVDELVKVMGRHKPENIVIEMARENQTTQKGQKNSRERMKRIEEGIKELGSQILKEHPVENTQLQNEKL
YLYYLQNGRDMYVDQELDINRLSDYDVDAIVPQSFLKDDSIDNKVLTRSDKNRGKSDNVPSEEVVKKMKNYWRQLLNAKLI
TQRKFDNLTKAERGGLSELDKAGFIKRQLVETRQITKHVAQILDSRMNTKYDENDKLIREVKVITLKSKLVSDFRKDFQFY
KVREINNYHHAHDAYLNAVVGTALIKKYPKLESEFVYGDYKVYDVRKMIAKSEQEIGKATAKYFFYSNIMNFFKTEITLAN
GEIRKRPLIETNGETGEIVWDKGRDFATVRKVLSMPQVNIVKKTEVQTGGFSKESILPKRNSDKLIARKKDWDPKKYGGFV
SPTVAYSVLVVAKVEKGKSKKLKSVKELLGITIMERSSFEKNPIDFLEAKGYKEVKKDLIIKLPKYSLFELENGRKRMLAS
AGELQKGNELALPSKYVNFLYLASHYEKLKGSPEDNEQKQLFVEQHKHYLDEIIEQISEFSKRVILADANLDKVLSAYNKH
RDKPIREQAENIIHLFTLTNLGAPAAFKYFDTTIDRKQYRSTKEVLDATLIHQSITGLYETRIDLSQLGGD

In another particular embodiment, the Cas9 variant having expanded PAM capabilities is SpCas9 (H840A) VRER, having the following amino acid sequence (with the V, R, E, R substitutions relative to the SpCas9 (H840A) of SEQ TD NO: 42 are shown in bold underline. In addition, the methionine residue in SpCas9 (11840) was removed for SpCas9 (H840A) VRER) (“SpCas9-VRER”). This SpCas9 variant possesses an altered PAM-specificity which recognizes a PAM of 5′-NGCG-3′ instead of the canonical PAM of 5′-NGG-3′:

SpCas9-VRER
(SEQ ID NO: 76)
DKKYSIGLDIGTNSVGWAVITDEYKVPSKKFKVLGNTDRHSIKKNLIGALLFDSGETAEATRLKRTARRRYTRRKNRICYL
QEIFSNEMAKVDDSFFHRLEESFLVEEDKKHERHPIFGNIVDEVAYHEKYPTIYHLRKKLVDSTDKADLRLIYLALAHMIK
FRGHFLIEGDLNPDNSDVDKLFIQLVQTYNQLFEENPINASGVDAKAILSARLSKSRRLENLIAQLPGEKKNGLFGNLIAL
SLGLTPNFKSNFDLAEDAKLQLSKDTYDDDLDNLLAQIGDQYADLFLAAKNLSDAILLSDILRVNTEITKAPLSASMIKRY
DEHHQDLTLLKALVRQQLPEKYKEIFFDQSKNGYAGYIDGGASQEEFYKFIKPILEKMDGTEELLVKLNREDLLRKQRTFD
NGSIPHQIHLGELHAILRRQEDFYPFLKDNREKIEKILTFRIPYYVGPLARGNSRFAWMTRKSEETITPWNFEEVVDKGAS
AQSFIERMTNFDKNLPNEKVLPKHSLLYEYFTVYNELTKVKYVTEGMRKPAFLSGEQKKAIVDLLFKTNRKVTVKQLKEDY
FKKIECFDSVEISGVEDRFNASLGTYHDLLKIIKDKDFLDNEENEDILEDIVLTLTLFEDREMIEERLKTYAHLFDDKVMK
QLKRRRYTGWGRLSRKLINGIRDKQSGKTILDFLKSDGFANRNFMQLIHDDSLTFKEDIQKAQVSGQGDSLHEHIANLAGS
PAIKKGILQTVKVVDELVKVMGRHKPENIVIEMARENQTTQKGQKNSRERMKRIEEGIKELGSQILKEHPVENTQLQNEKL
YLYYLQNGRDMYVDQELDINRLSDYDVDAIVPQSFLKDDSIDNKVLTRSDKNRGKSDNVPSEEVVKKMKNYWRQLLNAKLI
TQRKFDNLTKAERGGLSELDKAGFIKRQLVETRQITKHVAQILDSRMNTKYDENDKLIREVKVITLKSKLVSDFRKDFQFY
KVREINNYHHAHDAYLNAVVGTALIKKYPKLESEFVYGDYKVYDVRKMIAKSEQEIGKATAKYFFYSNIMNFFKTEITLAN
GEIRKRPLIETNGETGEIVWDKGRDFATVRKVLSMPQVNIVKKTEVQTGGFSKESILPKRNSDKLIARKKDWDPKKYGGFV
SPTVAYSVLVVAKVEKGKSKKLKSVKELLGITIMERSSFEKNPIDFLEAKGYKEVKKDLIIKLPKYSLFELENGRKRMLAS
ARELQKGNELALPSKYVNFLYLASHYEKLKGSPEDNEQKQLFVEQHKHYLDEIIEQISEFSKRVILADANLDKVLSAYNKH
RDKPIREQAENIIHLFTLTNLGAPAAFKYFDTTIDRKEYRSTKEVLDATLIHQSITGLYETRIDLSQLGGD

In yet particular embodiment, the Cas9 variant having expanded PAM capabilities is SpCas9-NG, as reported in Nishimasu et al., “Engineered CRISPR-Cas9 nuclease with expanded targeting space,” Science, 2018, 361: 1259-1262, which is incorporated herein by reference. SpCas9-NG (VRVRFRR), having the following amino acid sequence substitutions: R1335V, L1111R, D1135V, G1218R, E1219F, A1322R, and T1337R relative to the canonical SpCas9 sequence (SEQ TD NO: 5. This SpCas9 has a relaxed PAM specificity, i.e., with activity on a PAM of NGH (wherein H=A, T, or C). See Nishimasu et al., “Engineered CRISPR-Cas9 nuclease with expanded targeting space,” Science, 2018, 361: 1259-1262, which is incorporated herein by reference.

SpCas9-NG
(SEQ ID NO: 77)
MDKKYSIGLDIGTNSVGWAVITDEYKVPSKKFKVLGNTDRHSIKKNLIGALLFDSGETAEATRLKRTARRRYTRRKNRICY
LQEIFSNEMAKVDDSFFHRLEESFLVEEDKKHERHPIFGNIVDEVAYHEKYPTIYHLRKKLVDSTDKADLRLIYLALAHMI
KFRGHFLIEGDLNPDNSDVDKLFIQLVQTYNQLFEENPINASGVDAKAILSARLSKSRRLENLIAQLPGEKKNGLFGNLIA
LSLGLTPNFKSNFDLAEDAKLQLSKDTYDDDLDNLLAQIGDQYADLFLAAKNLSDAILLSDILRVNTEITKAPLSASMIKR
YDEHHQDLTLLKALVRQQLPEKYKEIFFDQSKNGYAGYIDGGASQEEFYKFIKPILEKMDGTEELLVKLNREDLLRKQRTF
DNGSIPHQIHLGELHAILRRQEDFYPFLKDNREKIEKILTFRIPYYVGPLARGNSRFAWMTRKSEETITPWNFEEVVDKGA
SAQSFIERMTNFDKNLPNEKVLPKHSLLYEYFTVYNELTKVKYVTEGMRKPAFLSGEQKKAIVDLLFKTNRKVTVKQLKED
YFKKIECFDSVEISGVEDRFNASLGTYHDLLKIIKDKDFLDNEENEDILEDIVLTLTLFEDREMIEERLKTYAHLFDDKVM
KQLKRRRYTGWGRLSRKLINGIRDKQSGKTILDFLKSDGFANRNFMQLIHDDSLTFKEDIQKAQVSGQGDSLHEHIANLAG
SPAIKKGILQTVKVVDELVKVMGRHKPENIVIEMARENQTTQKGQKNSRERMKRIEEGIKELGSQILKEHPVENTQLQNEK
LYLYYLQNGRDMYVDQELDINRLSDYDVDHIVPQSFLKDDSIDNKVLTRSDKNRGKSDNVPSEEVVKKMKNYWRQLLNAKL
ITQRKFDNLTKAERGGLSELDKAGFIKRQLVETRQITKHVAQILDSRMNTKYDENDKLIREVKVITLKSKLVSDFRKDFQF
YKVREINNYHHAHDAYLNAVVGTALIKKYPKLESEFVYGDYKVYDVRKMIAKSEQEIGKATAKYFFYSNIMNFFKTEITLA
NGEIRKRPLIETNGETGEIVWDKGRDFATVRKVLSMPQVNIVKKTEVQTGGFSKESIRPKRNSDKLIARKKDWDPKKYGGF
VSPTVAYSVLVVAKVEKGKSKKLKSVKELLGITIMERSSFEKNPIDFLEAKGYKEVKKDLIIKLPKYSLFELENGRKRMLA
SARFLQKGNELALPSKYVNFLYLASHYEKLKGSPEDNEQKQLFVEQHKHYLDEIIEQISEFSKRVILADANLDKVLSAYNK
HRDKPIREQAENIIHLFTLTNLGAPRAFKYFDTTIDRKVYRSTKEVLDATLIHQSITGLYETRIDLSQLGGD

In addition, any available methods may be utilized to obtain or construct a variant or mutant Cas9 protein. The term “mutation,” as used herein, refers to a substitution of a residue within a sequence, e.g., a nucleic acid or amino acid sequence, with another residue, or a deletion or insertion of one or more residues within a sequence. Mutations are typically described herein by identifying the original residue followed by the position of the residue within the sequence and by the identity of the newly substituted residue. Various methods for making the amino acid substitutions (mutations) provided herein are well known in the art, and are provided by, for example, Green and Sambrook, Molecular Cloning: A Laboratory Manual (4th ed., Cold Spring Harbor Laboratory Press, Cold Spring Harbor, N.Y. (2012)). Mutations can include a variety of categories, such as single base polymorphisms, microduplication regions, indel, and inversions, and is not meant to be limiting in any way. Mutations can include “loss-of-function” mutations which is the normal result of a mutation that reduces or abolishes a protein activity. Most loss-of-function mutations are recessive, because in a heterozygote the second chromosome copy carries an unmutated version of the gene coding for a fully functional protein whose presence compensates for the effect of the mutation. Mutations also embrace “gain-of-function” mutations, which is one which confers an abnormal activity on a protein or cell that is otherwise not present in a normal condition. Many gain-of-function mutations are in regulatory sequences rather than in coding regions, and can therefore have a number of consequences. For example, a mutation might lead to one or more genes being expressed in the wrong tissues, these tissues gaining functions that they normally lack. Because of their nature, gain-of-function mutations are usually dominant.

Mutations can be introduced into a reference Cas9 protein using site-directed mutagenesis. Older methods of site-directed mutagenesis known in the art rely on sub-cloning of the sequence to be mutated into a vector, such as an M13 bacteriophage vector, that allows the isolation of single-stranded DNA template. In these methods, one anneals a mutagenic primer (i.e., a primer capable of annealing to the site to be mutated but bearing one or more mismatched nucleotides at the site to be mutated) to the single-stranded template and then polymerizes the complement of the template starting from the 3′ end of the mutagenic primer. The resulting duplexes are then transformed into host bacteria and plaques are screened for the desired mutation. More recently, site-directed mutagenesis has employed PCR methodologies, which have the advantage of not requiring a single-stranded template. In addition, methods have been developed that do not require sub-cloning. Several issues must be considered when PCR-based site-directed mutagenesis is performed. First, in these methods it is desirable to reduce the number of PCR cycles to prevent expansion of undesired mutations introduced by the polymerase. Second, a selection must be employed in order to reduce the number of non-mutated parental molecules persisting in the reaction. Third, an extended-length PCR method is preferred in order to allow the use of a single PCR primer set. And fourth, because of the non-template-dependent terminal extension activity of some thermostable polymerases it is often necessary to incorporate an end-polishing step into the procedure prior to blunt-end ligation of the PCR-generated mutant product.

Mutations may also be introduced by directed evolution processes, such as phage-assisted continuous evolution (PACE) or phage-assisted noncontinuous evolution (PANCE). The term “phage-assisted continuous evolution (PACE),” as used herein, refers to continuous evolution that employs phage as viral vectors. The general concept of PACE technology has been described, for example, in International PCT Application, PCT/US2009/056194, filed Sep. 8, 2009, published as WO 2010/028347 on Mar. 11, 2010; International PCT Application, PCT/US2011/066747, filed Dec. 22, 2011, published as WO 2012/088381 on Jun. 28, 2012; U.S. application, U.S. Pat. No. 9,023,594, issued May 5, 2015, International PCT Application, PCT/US2015/012022, filed Jan. 20, 2015, published as WO 2015/134121 on Sep. 11, 2015, and International PCT Application, PCT/US2016/027795, filed Apr. 15, 2016, published as WO 2016/168631 on Oct. 20, 2016, the entire contents of each of which are incorporated herein by reference. Variant Cas9s may also be obtain by phage-assisted non-continuous evolution (PANCE),” which as used herein, refers to non-continuous evolution that employs phage as viral vectors. PANCE is a simplified technique for rapid in vivo directed evolution using serial flask transfers of evolving ‘selection phage’ (SP), which contain a gene of interest to be evolved, across fresh E. coli host cells, thereby allowing genes inside the host E. coli to be held constant while genes contained in the SP continuously evolve. Serial flask transfers have long served as a widely-accessible approach for laboratory evolution of microbes, and, more recently, analogous approaches have been developed for bacteriophage evolution. The PANCE system features lower stringency than the PACE system.

Any of the references noted above which relate to Cas9 or Cas9 equivalents are hereby incorporated by reference in their entireties, if not already stated so.

III. Adenosine Deaminases (or Adenine Deaminases)

In some embodiments, the disclosure provides base editors that comprise one or more adenosine deaminase domains. In some aspects, any of the disclosed base editors are capable of deaminating adenosine in a nucleic acid sequence (e.g., DNA or RNA). As one example, any of the base editors provided herein may be base editors, (e.g., adenine base editors). Without wishing to be bound by any particular theory, dimerization of adenosine deaminases (e.g., in cis or in trans) may improve the ability (e.g., efficiency) of the base editor to modify a nucleic acid base, for example to deaminate adenine.

Exemplary, non-limiting, embodiments of adenosine deaminases are provided herein. In some embodiments, the adenosine deaminase domain of any of the disclosed base editors comprises a single adenosine deaminase, or a monomer. In some embodiments, the adenosine deaminase domain comprises 2, 3, 4 or 5 adenosine deaminases. In some embodiments, the adenosine deaminase domain comprises two adenosine deaminases, or a dimer. In some embodiments, the deaminase domain comprises a dimer of an engineered (or evolved) deaminase and a wild-type deaminase, such as a wild-type E. coli deaminase. It should be appreciated that the mutations provided herein (e.g., mutations in ecTadA) may be applied to adenosine deaminases in other adenosine base editors, for example those provided in International Publication No. WO 2018/027078, published Aug. 2, 2018; International Application No PCT/US2019/033848, filed May 23, 2019, which published as International Publication No. WO 2019/226593 on Nov. 28, 2019; U.S. Patent Publication No. 2018/0073012, published Mar. 15, 2018, which issued as U.S. Pat. No. 10,113,163, on Oct. 30, 2018; U.S. Patent Publication No. 2017/0121693, published May 4, 2017, which issued as U.S. Pat. No. 10,167,457 on Jan. 1, 2019; International Publication No. WO 2017/070633, published Apr. 27, 2017; U.S. Patent Publication No. 2015/0166980, published Jun. 18, 2015; U.S. Pat. No. 9,840,699, issued Dec. 12, 2017; and U.S. Pat. No. 10,077,453, issued Sep. 18, 2018, and U.S. Provisional Application No. 62/835,490, filed Apr. 17, 2019; all of which are incorporated herein by reference in their entireties.

In some embodiments, any of the adenosine deaminases provided herein are capable of deaminating adenine, e.g., deaminating adenine in a deoxyadenosine residue of DNA. The adenosine deaminase may be derived from any suitable organism (e.g., E. coli). In some embodiments, the adenosine deaminase is a naturally-occurring adenosine deaminase that includes one or more mutations corresponding to any of the mutations provided herein (e.g., mutations in ecTadA). One of skill in the art will be able to identify the corresponding residue in any homologous protein and in the respective encoding nucleic acid by methods well known in the art, e.g., by sequence alignment and determination of homologous residues. Accordingly, one of skill in the art would be able to generate mutations in any naturally-occurring adenosine deaminase (e.g., having homology to ecTadA) that corresponds to any of the mutations described herein, e.g., any of the mutations identified in ecTadA. In some embodiments, the adenosine deaminase is derived from a prokaryote. In some embodiments, the adenosine deaminase is from a bacterium. In some embodiments, the adenosine deaminase is from Escherichia coli, Staphylococcus aureus, Salmonella typhi, Shewanella putrefaciens, Haemophilus influenzae, Caulobacter crescentus, or Bacillus subtilis. In some embodiments, the adenosine deaminase is from E. coli.

In some embodiments, the adenosine deaminase may comprise one or more substitutions that include R26G, V69A, V88A, A109S, T111R, D119N, H122N, Y147D, F149Y, T166I, D167N relative to TadA7.10 (SEQ ID NO: 79), or a substitution at a corresponding amino acid in another adenosine deaminase. In some embodiments, the adenosine deaminase comprises T111R, D119N, and F149Y substitutions in TadA7.10 (SEQ ID NO: 79), or a corresponding mutation in another adenosine deaminase. In particular embodiments, the adenosine deaminase comprises T111R, D119N, and F149Y substitutions, and further comprises at least one substitution selected from R26C, V88A, A109S, H122N, T166I, and D167N, in TadA7.10 (SEQ ID NO: 79), or a corresponding mutation in another adenosine deaminase.

In some embodiments, the adenosine deaminase comprises A109S, T111R, D119N, H122N, F149Y, T166I, and D167N substitutions in TadA7.10 (SEQ ID NO: 79), or a corresponding mutation in another adenosine deaminase. In some embodiments, the adenosine deaminase comprises R26C, D108W, T111R, D119N, and F149Y substitutions in TadA7.10 (SEQ ID NO: 79), or a corresponding mutation in another adenosine deaminase. In some embodiments, the adenosine deaminase comprises V88A, D108W, T111R, D119N, and F149Y substitutions in TadA7.10 (SEQ ID NO: 79), or a corresponding mutation in another adenosine deaminase. In some embodiments, the adenosine deaminase further comprises a Y147D substitution in TadA7.10 (SEQ ID NO: 79), or a corresponding mutation in another adenosine deaminase.

In some embodiments, the adenosine deaminase comprises A109S, T111R, D119N, H122N, Y147D, F149Y, T166I and D167N substitutions in TadA7.10 (SEQ ID NO: 79), or a corresponding mutation in another adenosine deaminase. In some embodiments, the adenosine deaminase comprises TadA-8e. In some embodiments, the adenosine deaminase comprises A109S, T111R, D119N, H122N, Y147D, F149Y, T166I and D167N in TadA7.10 (SEQ ID NO: 79), or a corresponding mutation in another adenosine deaminase. In some embodiments, the adenosine deaminase further comprises at least one substitution selected from K20A, R21A, V82G, and V106W in TadA7.10 (SEQ ID NO: 79), or a corresponding mutation in another adenosine deaminase. In certain embodiments, the adenosine deaminase comprises V106W, A109S, T111R, D119N, H122N, Y147D, F149Y, T166I and D167N substitutions in TadA7.10 (SEQ ID NO: 79), or a corresponding mutation in another adenosine deaminase. In some embodiments, the adenosine deaminase comprises TadA-8e(V106W). It should be appreciated, however, that additional deaminases may similarly be aligned to identify homologous amino acid residues that may be mutated as provided herein.

It should be appreciated that any of the mutations provided herein (e.g., based on the ecTadA amino acid sequence of SEQ ID NO: 78) may be introduced into other adenosine deaminases, such as S. aureus TadA (saTadA), or other adenosine deaminases (e.g., bacterial adenosine deaminases), such as those sequences provided below. It would be apparent to the skilled artisan how to identify amino acid residues from other adenosine deaminases that are homologous to the mutated residues in ecTadA. Thus, any of the mutations identified in ecTadA may be made in other adenosine deaminases that have homologous amino acid residues. It should also be appreciated that any of the mutations provided herein may be made individually or in any combination in ecTadA or another adenosine deaminase.

Exemplary adenosine deaminase variants of the disclosure are described below. In certain embodiments, the adenosine deaminase domain comprises an adenosine deaminase that has a sequence with at least 80%, at least 85%, at least 90%, at least 95%, at least 98%, at least 99%, or at least 99.5% sequence identity to one of the following:

E. coli TadA
(SEQ ID NO: 78)
MSEVEFSHEYWMRHALTLAKRAWDEREVPVGAVLV
HNNRVIGEGWNRPIGRHDPTAHAEIMALRQGGLVM
QNYRLIDATLYVTLEPCVMCAGAMIHSRIGRVVFG
ARDAKTGAAGSLMDVLHHPGMNHRVEITEGILADE
CAALLSDFFRMRRQEIKAQKKAQSSTD
E. coli TadA 7.10
(SEQ ID NO: 79)
MSEVEFSHEYWMRHALTLAKRARDEREVPVGAVLV
LNNRVIGEGWNRAIGLHDPTAHAEIMALRQGGLVM
QNYRLIDATLYVTFEPCVMCAGAMIHSRIGRVVFG
VRNAKTGAAGSLMDVLHYPGMNHRVEITEGILADE
CAALLCYFFRMPRQVFNAQKKAQSSTD
E. coli TadA* 7.10
(SEQ ID NO: 403)
SEVEFSHEYWMRHALTLAKRARDEREVPVGAVLVL
NNRVIGEGWNRAIGLHDPTAHAEIMALRQGGLVMQ
NYRLIDATLYVTFEPCVMCAGAMIHSRIGRVVFGV
RNAKTGAAGSLMDVLHYPGMNHRVEITEGILADEC
AALLCYFFRMPRQVFNAQKKAQSSTD
ABE7.10 TadA* monomer
DNA sequence
(SEQ ID NO: 404)
TCTGAGGTGGAGTTTTCCCACGAGTACTGGATGAG
ACATGCCCTGACCCTGGCCAAGAGGGCACGCGATG
AGAGGGAGGTGCCTGTGGGAGCCGTGCTGGTGCTG
AACAATAGAGTGATCGGCGAGGGCTGGAACAGAGC
CATCGGCCTGCACGACCCAACAGCCCATGCCGAAA
TTATGGCCCTGAGACAGGGCGGCCTGGTCATGCAG
AACTACAGACTGATTGACGCCACCCTGTACGTGAC
ATTCGAGCCTTGCGTGATGTGCGCCGGCGCCATGA
TCCACTCTAGGATCGGCCGCGTGGTGTTTGGCGTG
AGGAACGCAAAAACCGGCGCCGCAGGCTCCCTGAT
GGACGTGCTGCACTACCCCGGCATGAATCACCGCG
TCGAAATTACCGAGGGAATCCTGGCAGATGAATGT
GCCGCCCTGCTGTGCTATTTCTTTCGGATGCCTAG
ACAGGTGTTCAATGCTCAGAAGAAGGCCCAGAGCT
CCACCGAC
E. coli TadA 7.10 (V106W)
(SEQ ID NO: 80)
MSEVEFSHEYWMRHALTLAKRARDEREVPVGAVLV
LNNRVIGEGWNRAIGLHDPTAHAEIMALRQGGLVM
QNYRLIDATLYVTFEPCVMCAGAMIHSRIGRVVFG
WRNAKTGAAGSLMDVLHYPGMNHRVEITEGILADE
CAALLCYFFRMPRQVFNAQKKAQSSTD
Staphylococcus aureus TadA
(SEQ ID NO: 81)
MGSHMTNDIYFMTLAIEEAKKAAQLGEVPIGAIIT
KDDEVIARAHNLRETLQQ
PTAHAEHIAIERAAKVLGSWRLEGCTLYVTLEPCV
MCAGTIVMSRIPRVVYGADDPKGGCSGSLMNLLQQ
SNFNHRAIVDKGVLKEACSTLLTTFFKNLRANKKS
TN
Streptococcus pyogenes (S. pyogenes) TadA
(SEQ ID NO: 3238)
MPYSLEEQTYFMQEALKEAEKSLQKAEIPIGCVIV
KDGEIIGRGHNAREESNQAIMHAEIMAINEANAHE
GNWRLLDTTLFVTIEPCVMCSGAIGLARIPHVIYG
ASNQKFGGADSLYQILTDERLNHRVQVERGLLAAD
CANIMQTFFRQGRERKKIAKHLIKEQSDPFD
Bacillus subtilis TadA
(SEQ ID NO: 82)
MTQDELYMKEAIKEAKKAEEKGEVPIGAVLVINGE
IIARAHNLRETEQRSIAHAEMLVIDEACKALGTWR
LEGATLYVTLEPCPMCAGAVVLSRVEKVVFGAFDP
KGGCSGTLMNLLQEERFNHQAEVVSGVLEEECGGM
LSAFFRELRKKKKAARKNLSE
Salmonella typhimurium TadA
(SEQ ID NO: 83)
MPPAFITGVTSLSDVELDHEYWMRHALTLAKRAWD
EREVPVGAVLVHNHRVIGEGWNRPIGRHDPTAHAE
IMALRQGGLVLQNYRLLDTTLYVTLEPCVMCAGAM
VHSRIGRVVFGARDAKTGAAGSLIDVLHHPGMNHR
VEIIEGVLRDECATLLSDFFRMRRQEIKALKKADR
AEGAGPAV
Shewanella putrefaciens TadA
(SEQ ID NO: 84)
MDEYWMQVAMQMAEKAEAAGEVPVGAVLVKDGQQI
ATGYNLSISQHDPTAHAEILCLRSAGKKLENYRLL
DATLYITLEPCAMCAGAMVHSRIARVVYGARDEKT
GAAGTVVNLLQHPAFNHQVEVTSGVLAEACSAQLS
RFFKRRRDEKKALKLAQRAQQGIE
Haemophilus influenzae F3 031 Tad A
(SEQ ID NO: 85)
MDAAKVRSEFDEKMMRYALELADKAEALGEIPVGA
VLVDDARNIIGEGWNLSIVQSDPTAHAEIIALRNG
AKNIQNYRLLNSTLYVTLEPCTMCAGAILHSRIKR
LVFGASDYKTGAIGSRFHFFDDYKMNHTLEITSGV
LAEECSQKLSTFFQKRREEKKIEKALLKSLSDK
Caulobacter crescentus TadA
(SEQ ID NO: 86)
MRTDESEDQDHRMMRLALDAARAAAEAGETPVGAVI
LDPSTGEVIATAGNGPIAAHDPTAHAEIAAMRAAA
AKLGNYRLTDLTLVVTLEPCAMCAGAISHARIGRV
VFGADDPKGGAVVHGPKFFAQPTCHWRPEVTGGVL
ADESADLLRGFFRARRKAKI
Geobacter sulfurreducens TadA
(SEQ ID NO: 87)
MSSLKKTPIRDDAYWMGKAIREAAKAAARDEVPIG
AVIVRDGAVIGRGHNLREGSNDPSAHAEMIAIRQA
ARRSANWRLTGATLYVTLEPCLMCMGAIILARLER
VVFGCYDPKGAAGSLYDLSADPRLNHQVRLSPGVC
QEECGTMLSDFFRDLRRRKKAKATPALFIDERKVP
PEP

In some embodiments, the adenosine deaminase domain comprises an N-terminal truncated E. coli TadA. In certain embodiments, the adenosine deaminase comprises the amino acid sequence:

(SEQ ID NO: 78)
MSEVEFSHEYWMRHALTLAKRAWDEREVPVGAVLV
HNNRVIGEGWNRPIGRHDPTAHAEIMALRQGGLVM
QNYRLIDATLYVTLEPCVMCAGAMIHSRIGRVVFG
ARDAKTGAAGSLMDVLHHPGMNHRVEITEGILADE
CAALLSDFFRMRRQEIKAQKKAQSSTD.

In some embodiments, the TadA deaminase is a full-length E. coli TadA deaminase (ecTadA). For example, in certain embodiments, the adenosine deaminase domain comprises a deaminase that comprises the amino acid sequence:

(SEQ ID NO: 89)
MRRAFITGVFFLSEVEFSHEYWMRHALTLAKRAWD
EREVPVGAVLVHNNRVIGEGWNRPIGRHDPTAHAE
IMALRQGGLVMQNYRLIDATLYVTLEPCVMCAGAM
IHSRIGRVVFGARDAKTGAAGSLMDVLHHPGMNHR
VEITEGILADECAALLSDFFRMRRQEIKAQKKAQS
STD
ABE8 TadA* monomer
DNA sequence
(SEQ ID NO: 90)
TCTGAGGTGGAGTTTTCCCACGAGTACTGGATGAG
ACATGCCCTGACCCTGGCCAAGAGGGCACGGGATG
AGAGGGAGGTGCCTGTGGGAGCCGTGCTGGTGCTG
AACAATAGAGTGATCGGCGAGGGCTGGAACAGAGC
CATCGGCCTGCACGACCCAACAGCCCATGCCGAAA
TTATGGCCCTGAGACAGGGCGGCCTGGTCATGCAG
AACTACAGACTGATTGACGCCACCCTGTACGTGAC
ATTCGAGCCTTGCGTGATGTGCGCCGGCGCCATGA
TCCACTCTAGGATCGGCCGCGTGGTGTTTGGCGTG
AGGAACTCAAAAAGAGGCGCCGCAGGCTCCCTGAT
GAACGTGCTGAACTACCCCGGCATGAATCACCGCG
TCGAAATTACCGAGGGAATCCTGGCAGATGAATGT
GCCGCCCTGCTGTGCGATTTCTATCGGATGCCTAG
ACAGGTGTTCAATGCTCAGAAGAAGGCCCAGAGCT
CCATCAAC
ABE8 TadA* monomer
Amino Acid Sequence
(SEQ ID NO: 91)
MSEVEFSHEYWMRHALTLAKRARDEREVPVGAVLV
LNNRVIGEGWNRAIGLHDPTAHAEIMALRQGGLVM
QNYRLIDATLYVTFEPCVMCAGAMIHSRIGRVVFG
VRNSKRGAAGSLMNVLNYPGMNHRVEITEGILADE
CAALLCDFYRMPRQVFNAQKKAQSSIN

In other aspects, the disclosure provides adenine base editors with broadened target sequence compatibility. In general, native ecTadA deaminates the adenine in the sequence UAC (e.g., the target sequence) of the anticodon loop of tRNA^{Arg}. Without wishing to be bound by any particular theory, in order to expand the utility of ABEs comprising one or more ecTadA deaminases, such as any of the adenosine deaminases provided herein, the adenosine deaminase proteins were optimized to recognize a wide variety of target sequences within the protospacer sequence without compromising the editing efficiency of the adenosine nucleobase editor complex. In some embodiments, the target sequence is an A in the center of a 5′-NAN-3′ sequence, wherein N is T, C, G, or A. In some embodiments, the target sequence comprises 5′-TAC-3′. In some embodiments, the target sequence comprises 5′-GAA-3′.

Any two or more of the adenosine deaminases described herein may be connected to one another (e.g., by a linker) within an adenosine deaminase domain of the base editors provided herein. For instance, the base editors provided herein may contain only two adenosine deaminases. In some embodiments, the adenosine deaminases are the same. In some embodiments, the adenosine deaminases are any of the adenosine deaminases provided herein. In some embodiments, the adenosine deaminases are different. In some embodiments, the first adenosine deaminase is any of the adenosine deaminases provided herein, and the second adenosine is any of the adenosine deaminases provided herein, but is not identical to the first adenosine deaminase. In some embodiments, the base editor comprises two adenosine deaminases (e.g., a first adenosine deaminase and a second adenosine deaminase). In some embodiments, the base editor comprises a first adenosine deaminase and a second adenosine deaminase. In some embodiments, the first adenosine deaminase is N-terminal to the second adenosine deaminase in the base editor. In some embodiments, the first adenosine deaminase is C-terminal to the second adenosine deaminase in the base editor. In some embodiments, the first adenosine deaminase and the second deaminase are fused directly or via a linker.

In some embodiments, the adenosine deaminase domain comprises an adenosine deaminase that comprises an amino acid sequence that is at least 60%, at least 65%, at least 70%, at least 75%, at least 80%, at least 85%, at least 90%, at least 95%, at least 96%, at least 97%, at least 98%, at least 99%, or at least 99.5% identical to any one of the amino acid sequences set forth in any one of SEQ ID NOs: 78-91, or to any of the adenosine deaminases provided herein. In certain embodiments, the adenosine deaminase comprises an amino acid sequence that is at least 80%, at least 85%, at least 90%, at least 95%, at least 96%, at least 97%, at least 98%, at least 99%, or at least 99.5% identical to the amino acid sequence of TadA7.10 (SEQ ID NO: 403). It should be appreciated that adenosine deaminases provided herein may include one or more mutations (e.g., any of the mutations provided herein). The disclosure provides adenosine deaminases with a certain percent identity plus any of the mutations or combinations thereof described herein. In some embodiments, the adenosine deaminase comprises an amino acid sequence that has 1, 2, 3, 4, 5, 6, 7, 8, 9, 10, 11, 12, 13, 14, 15, 16, 17, 18, 19, 20, 21, 22, 21, 24, 25, 26, 27, 28, 29, 30, 31, 32, 33, 34, 35, 36, 37, 38, 39, 40, 41, 42, 43, 44, 45, 46, 47, 48, 49, 50, or more mutations compared to any one of the amino acid sequences set forth in SEQ ID NOs: 78-91, and 403-404 (e.g., TadA7.10), or any of the adenosine deaminases provided herein. In some embodiments, the adenosine deaminase comprises an amino acid sequence that has at least 5, at least 10, at least 15, at least 20, at least 25, at least 30, at least 35, at least 40, at least 45, at least 50, at least 60, at least 70, at least 80, at least 90, at least 100, at least 110, at least 120, at least 130, at least 140, at least 150, at least 160, or at least 170 identical contiguous amino acid residues as compared to any one of the amino acid sequences set forth in SEQ ID NOs: 78-91, and 403-404 (e.g., TadA7.10), or any of the adenosine deaminases provided herein.

In some embodiments, the adenosine deaminase comprises TadA 7.10, whose sequence is set forth as SEQ ID NO: 79, or a variant thereof. TadA7.10 comprises the following mutations in wild-type ecTadA: W23R, H36L, P48A, R51L, L84F, A106V, D108N, H123Y, S146C, D147Y, R152P, E155V, I156F, and K157N.

In some embodiments, the adenosine deaminase is at least 50%, at least 55%, at least 60%, at least 65%, at least 70%, at least 75% at least 80%, at least 85%, at least 90%, at least 95%, at least 96%, at least 97%, at least 98%, at least 99%, or at least 99.5% identical to a naturally-occurring adenosine deaminase, e.g., E. coli TadA 7.10 of SEQ ID NO: 79. In some embodiments, the adenosine deaminase is from a bacterium, such as, E. coli, S. aureus, S. typhi, S. putrefaciens, H. influenzae, or C. crescentus. In some embodiments, the adenosine deaminase is a TadA deaminase. In some embodiments, the TadA deaminase is an E. coli TadA deaminase (ecTadA). In some embodiments, the TadA deaminase is a truncated E. coli TadA deaminase. For example, the truncated ecTadA may be missing one or more N-terminal or C-terminal amino acids relative to a full-length ecTadA. In some embodiments, the truncated ecTadA may be missing 1, 2, 3, 4, 5, 6, 7, 8, 9, 10, 11, 12, 13, 14, 15, 6, 17, 18, 19, or 20 N-terminal amino acid residues relative to the full length ecTadA. In some embodiments, the truncated ecTadA may be missing 1, 2, 3, 4, 5, 6, 7, 8, 9, 10, 11, 12, 13, 14, 15, 6, 17, 18, 19, or 20 C-terminal amino acid residues relative to the full length ecTadA. In some embodiments, the ecTadA deaminase does not comprise an N-terminal methionine.

In some embodiments, the TadA 7.10 of SEQ ID NO: 79 comprises an N-terminal methionine. It should be appreciated that the amino acid numbering scheme relating to the mutations in TadA 7.10 may be based on the TadA sequence of SEQ ID NO: 78, which contains an N-terminal methionine.

In some embodiments, the adenosine deaminase comprises a D108X mutation in ecTadA SEQ ID NO: 89, or a corresponding mutation in another adenosine deaminase, where X indicates any amino acid other than the corresponding amino acid in the wild-type adenosine deaminase. In some embodiments, the adenosine deaminase comprises a D108G, D108N, D108V, D108A, or D108Y mutation in ecTadA SEQ ID NO: 89, or a corresponding mutation in another adenosine deaminase. In some embodiments, the adenosine deaminase comprises a D108N mutation in ecTadA SEQ ID NO: 89, or a corresponding mutation in another adenosine deaminase. It should be appreciated, however, that additional deaminases may similarly be aligned to identify homologous amino acid residues that can be mutated as provided herein.

In some embodiments, the adenosine deaminase comprises an A106X mutation in ecTadA SEQ ID NO: 78, or a corresponding mutation in another adenosine deaminase, where X indicates any amino acid other than the corresponding amino acid in the wild-type adenosine deaminase. In some embodiments, the adenosine deaminase comprises an A106V mutation in ecTadA SEQ ID NO: 78, or a corresponding mutation in another adenosine deaminase.

In some embodiments, the adenosine deaminase comprises a E155X mutation in ecTadA SEQ ID NO: 78, or a corresponding mutation in another adenosine deaminase, where the presence of X indicates any amino acid other than the corresponding amino acid in the wild-type adenosine deaminase. In some embodiments, the adenosine deaminase comprises a E155D, E155G, or E155V mutation in ecTadA SEQ ID NO: 78, or a corresponding mutation in another adenosine deaminase. In some embodiments, the adenosine deaminase comprises a E155V mutation in ecTadA SEQ ID NO: 78, or a corresponding mutation in another adenosine deaminase).

In some embodiments, the adenosine deaminase comprises a D147X mutation in ecTadA SEQ ID NO: 78, or a corresponding mutation in another adenosine deaminase, where the presence of X indicates any amino acid other than the corresponding amino acid in the wild-type adenosine deaminase. In some embodiments, the adenosine deaminase comprises a D147Y mutation in ecTadA SEQ ID NO: 78, or a corresponding mutation in another adenosine deaminase.

In some embodiments, an adenosine deaminase comprises the following group of mutations (groups of mutations are separated by a “;”) in ecTadA SEQ ID NO: 78, or corresponding mutations in another adenosine deaminase: D108N and A106V; D108N and E155V; D108N and D147Y; A106V and E155V; A106V and D147Y; E155V and D147Y; D108N, A106V, and E55V; D108N, A106V, and D147Y; D108N, E55V, and D147Y; A106V, E55V, and D147Y; and D108N, A106V, E55V, and D147Y. It should be appreciated, however, that any combination of corresponding mutations provided herein may be made in an adenosine deaminase (e.g., ecTadA). In some embodiments, an adenosine deaminase comprises one or more of the mutations provided herein, which identifies individual mutations and combinations of mutations made in ecTadA. In some embodiments, an adenosine deaminase comprises any mutation or combination of mutations provided herein.

In some embodiments, the adenosine deaminase comprises an L84X mutation in ecTadA SEQ ID NO: 78, or a corresponding mutation in another adenosine deaminase, where X indicates any amino acid other than the corresponding amino acid in the wild-type adenosine deaminase. In some embodiments, the adenosine deaminase comprises an L84F mutation in ecTadA SEQ ID NO: 78, or a corresponding mutation in another adenosine deaminase.

In some embodiments, the adenosine deaminase comprises an H123X mutation in ecTadA SEQ ID NO: 78, or a corresponding mutation in another adenosine deaminase, where X indicates any amino acid other than the corresponding amino acid in the wild-type adenosine deaminase. In some embodiments, the adenosine deaminase comprises an H123Y mutation in ecTadA SEQ ID NO: 78, or a corresponding mutation in another adenosine deaminase.

In some embodiments, the adenosine deaminase comprises an I156X mutation in ecTadA SEQ ID NO: 78, or a corresponding mutation in another adenosine deaminase, where X indicates any amino acid other than the corresponding amino acid in the wild-type adenosine deaminase. In some embodiments, the adenosine deaminase comprises an I156F mutation in ecTadA SEQ ID NO: 78, or a corresponding mutation in another adenosine deaminase.

In some embodiments, the adenosine deaminase comprises one, two, three, four, five, six, or seven mutations selected from the group consisting of L84X, A106X, D108X, H123X, D147X, E155X, and I156X in ecTadA SEQ ID NO: 78, or a corresponding mutation or mutations in another adenosine deaminase, where X indicates the presence of any amino acid other than the corresponding amino acid in the wild-type adenosine deaminase.

In some embodiments, the adenosine deaminase comprises one, two, three, four, five, six, or seven mutations selected from the group consisting of L84F, A106V, D108N, H123Y, D147Y, E155V, and I156F in ecTadA SEQ ID NO: 78, or a corresponding mutation or mutations in another adenosine deaminase. In some embodiments, the adenosine deaminase comprises one, two, three, four, five, or six mutations selected from the group consisting of S2A, I49F, A106V, D108N, D147Y, and E155V in ecTadA SEQ ID NO: 78, or a corresponding mutation or mutations in another adenosine deaminase. In some embodiments, the adenosine deaminase comprises one, two, three, four, or five, mutations selected from the group consisting of H8Y, A106T, D108N, N127S, and K160S in ecTadA SEQ ID NO: 78, or a corresponding mutation or mutations in another adenosine deaminase.

In some embodiments, the adenosine deaminase comprises an A142X mutation in ecTadA SEQ ID NO: 78, or a corresponding mutation in another adenosine deaminase, where X indicates any amino acid other than the corresponding amino acid in the wild-type adenosine deaminase. In some embodiments, the adenosine deaminase comprises an A142N, A142D, A142G, mutation in ecTadA SEQ ID NO: 78, or a corresponding mutation in another adenosine deaminase. In some embodiments, the adenosine deaminase comprises an A142N mutation in ecTadA SEQ ID NO: 78, or a corresponding mutation in another adenosine deaminase.

In some embodiments, the adenosine deaminase comprises an H36X mutation in ecTadA SEQ ID NO: 78, or a corresponding mutation in another adenosine deaminase, where X indicates any amino acid other than the corresponding amino acid in the wild-type adenosine deaminase. In some embodiments, the adenosine deaminase comprises an H36L mutation in ecTadA SEQ ID NO: 78, or a corresponding mutation in another adenosine deaminase.

In some embodiments, the adenosine deaminase comprises an N37X mutation in ecTadA SEQ ID NO: 78, or a corresponding mutation in another adenosine deaminase, where X indicates any amino acid other than the corresponding amino acid in the wild-type adenosine deaminase. In some embodiments, the adenosine deaminase comprises an N37T, or N37S mutation in ecTadA SEQ ID NO: 78, or a corresponding mutation in another adenosine deaminase. In some embodiments, the adenosine deaminase comprises a N37S mutation in ecTadA SEQ ID NO: 78, or a corresponding mutation in another adenosine deaminase.

In some embodiments, the adenosine deaminase comprises an P48X mutation in ecTadA SEQ ID NO: 78, or a corresponding mutation in another adenosine deaminase, where X indicates any amino acid other than the corresponding amino acid in the wild-type adenosine deaminase. In some embodiments, the adenosine deaminase comprises an P48T, P48S, P48A, or P48L mutation in ecTadA SEQ ID NO: 78, or a corresponding mutation in another adenosine deaminase. In some embodiments, the adenosine deaminase comprises a P48T mutation in ecTadA SEQ ID NO: 78, or a corresponding mutation in another adenosine deaminase. In some embodiments, the adenosine deaminase comprises a P48S mutation in ecTadA SEQ ID NO: 78, or a corresponding mutation in another adenosine deaminase. In some embodiments, the adenosine deaminase comprises a P48A mutation in ecTadA SEQ ID NO: 78, or a corresponding mutation in another adenosine deaminase.

In some embodiments, the adenosine deaminase comprises an R51X mutation in ecTadA SEQ ID NO: 78, or a corresponding mutation in another adenosine deaminase, where X indicates any amino acid other than the corresponding amino acid in the wild-type adenosine deaminase. In some embodiments, the adenosine deaminase comprises an R51H, or R51L mutation in ecTadA SEQ ID NO: 78, or a corresponding mutation in another adenosine deaminase. In some embodiments, the adenosine deaminase comprises a R51L mutation in ecTadA SEQ ID NO: 78, or a corresponding mutation in another adenosine deaminase.

In some embodiments, the adenosine deaminase comprises an S146X mutation in ecTadA SEQ ID NO: 78, or a corresponding mutation in another adenosine deaminase, where X indicates any amino acid other than the corresponding amino acid in the wild-type adenosine deaminase. In some embodiments, the adenosine deaminase comprises an S146R, or S146C mutation in ecTadA SEQ ID NO: 78, or a corresponding mutation in another adenosine deaminase. In some embodiments, the adenosine deaminase comprises a S146C mutation in ecTadA SEQ ID NO: 78, or a corresponding mutation in another adenosine deaminase.

In some embodiments, the adenosine deaminase comprises an K157X mutation in ecTadA SEQ ID NO: 78, or a corresponding mutation in another adenosine deaminase, where X indicates any amino acid other than the corresponding amino acid in the wild-type adenosine deaminase. In some embodiments, the adenosine deaminase comprises a K157N mutation in ecTadA SEQ ID NO: 78, or a corresponding mutation in another adenosine deaminase.

In some embodiments, the adenosine deaminase comprises an W23X mutation in ecTadA SEQ ID NO: 78, or a corresponding mutation in another adenosine deaminase, where X indicates any amino acid other than the corresponding amino acid in the wild-type adenosine deaminase. In some embodiments, the adenosine deaminase comprises a W23R, or W23L mutation in ecTadA SEQ ID NO: 78, or a corresponding mutation in another adenosine deaminase. In some embodiments, the adenosine deaminase comprises a W23R mutation in ecTadA SEQ ID NO: 78, or a corresponding mutation in another adenosine deaminase. In some embodiments, the adenosine deaminase comprises a W23L mutation in ecTadA SEQ ID NO: 78, or a corresponding mutation in another adenosine deaminase.

In some embodiments, the adenosine deaminase comprises an R152X mutation in ecTadA SEQ ID NO: 78, or a corresponding mutation in another adenosine deaminase, where X indicates any amino acid other than the corresponding amino acid in the wild-type adenosine deaminase. In some embodiments, the adenosine deaminase comprises a R152P, or R52H mutation in ecTadA SEQ ID NO: 78, or a corresponding mutation in another adenosine deaminase. In some embodiments, the adenosine deaminase comprises a R152P mutation in ecTadA SEQ ID NO: 78, or a corresponding mutation in another adenosine deaminase. In some embodiments, the adenosine deaminase comprises a R152H mutation in ecTadA SEQ ID NO: 78, or a corresponding mutation in another adenosine deaminase.

In some embodiments, the adenosine deaminase comprises an R26X mutation in ecTadA SEQ ID NO: 78, or a corresponding mutation in another adenosine deaminase, where X indicates any amino acid other than the corresponding amino acid in the wild-type adenosine deaminase. In some embodiments, the adenosine deaminase comprises a R26G mutation in ecTadA SEQ ID NO: 78, or a corresponding mutation in another adenosine deaminase.

In some embodiments, the adenosine deaminase comprises an I49X mutation in ecTadA SEQ ID NO: 78, or a corresponding mutation in another adenosine deaminase, where X indicates any amino acid other than the corresponding amino acid in the wild-type adenosine deaminase. In some embodiments, the adenosine deaminase comprises a I49V mutation in ecTadA SEQ ID NO: 78, or a corresponding mutation in another adenosine deaminase.

In some embodiments, the adenosine deaminase comprises an N72X mutation in ecTadA SEQ ID NO: 78, or a corresponding mutation in another adenosine deaminase, where X indicates any amino acid other than the corresponding amino acid in the wild-type adenosine deaminase. In some embodiments, the adenosine deaminase comprises a N72D mutation in ecTadA SEQ ID NO: 78, or a corresponding mutation in another adenosine deaminase.

In some embodiments, the adenosine deaminase comprises an S97X mutation in ecTadA SEQ ID NO: 78, or a corresponding mutation in another adenosine deaminase, where X indicates any amino acid other than the corresponding amino acid in the wild-type adenosine deaminase. In some embodiments, the adenosine deaminase comprises a S97C mutation in ecTadA SEQ ID NO: 78, or a corresponding mutation in another adenosine deaminase.

In some embodiments, the adenosine deaminase comprises an G125X mutation in ecTadA SEQ ID NO: 78, or a corresponding mutation in another adenosine deaminase, where X indicates any amino acid other than the corresponding amino acid in the wild-type adenosine deaminase. In some embodiments, the adenosine deaminase comprises a G125A mutation in ecTadA SEQ ID NO: 78, or a corresponding mutation in another adenosine deaminase.

In some embodiments, the adenosine deaminase comprises an K161X mutation in ecTadA SEQ ID NO: 78, or a corresponding mutation in another adenosine deaminase, where X indicates any amino acid other than the corresponding amino acid in the wild-type adenosine deaminase. In some embodiments, the adenosine deaminase comprises a K161T mutation in ecTadA SEQ ID NO: 78, or a corresponding mutation in another adenosine deaminase.

In some embodiments, the adenosine deaminase comprises one or more of a W23X, H36X, N37X, P48X, I49X, R51X, N72X, L84X, S97X, A106X, D108X, H123X, G125X, A142X, S146X, D147X, R152X, E155X, I156X, K157X, and/or K161X mutation in ecTadA SEQ ID NO: 78, or one or more corresponding mutations in another adenosine deaminase, where the presence of X indicates any amino acid other than the corresponding amino acid in the wild-type adenosine deaminase. In some embodiments, the adenosine deaminase comprises one or more of W23L, W23R, H36L, P48S, P48A, R51L, L84F, A106V, D108N, H123Y, A142N, S146C, D147Y, R152P, E155V, I156F, and/or K157N mutation in ecTadA SEQ ID NO: 78, or one or more corresponding mutations in another adenosine deaminase. In some embodiments, the adenosine deaminase comprises one or more of the mutations provided herein corresponding to ecTadA SEQ ID NO: 78, or one or more corresponding mutations in another adenosine deaminase.

In some embodiments, the adenosine deaminase comprises or consists of one or two mutations selected from A106X and D108X in ecTadA SEQ ID NO: 78, or a corresponding mutation or mutations in another adenosine deaminase, where X indicates the presence of any amino acid other than the corresponding amino acid in the wild-type adenosine deaminase. In some embodiments, the adenosine deaminase comprises or consists of one or two mutations selected from A106V and D108N in ecTadA SEQ ID NO: 78, or a corresponding mutation or mutations in another adenosine deaminase.

In some embodiments, the adenosine deaminase comprises or consists of one, two, three, or four mutations selected from A106X, D108X, D147X, and E155X in ecTadA SEQ ID NO: 78, or a corresponding mutation or mutations in another adenosine deaminase, where X indicates the presence of any amino acid other than the corresponding amino acid in the wild-type adenosine deaminase. In some embodiments, the adenosine deaminase comprises or consists of one, two, three, or four mutations selected from A106V, D108N, D147Y, and E155V in ecTadA SEQ ID NO: 78, or a corresponding mutation or mutations in another adenosine deaminase. In some embodiments, the adenosine deaminase comprises or consists of a A106V, D108N, D147Y, and E155V mutation in ecTadA SEQ ID NO: 78, or corresponding mutations in another adenosine deaminase.

In some embodiments, the adenosine deaminase comprises or consists of one, two, three, four, five, six, or seven mutations selected from L84X, A106X, D108X, H123X, D147X, E155X, and I156X in ecTadA SEQ ID NO: 78, or a corresponding mutation or mutations in another adenosine deaminase, where X indicates the presence of any amino acid other than the corresponding amino acid in the wild-type adenosine deaminase. In some embodiments, the adenosine deaminase comprises or consists of one, two, three, four, five, six, or seven mutations selected from L84F, A106V, D108N, H123Y, D147Y, E155V, and I156F in ecTadA SEQ ID NO: 78, or a corresponding mutation or mutations in another adenosine deaminase. In some embodiments, the adenosine deaminase comprises or consists of a L84F, A106V, D108N, H123Y, D147Y, E155V, and I156F mutation in ecTadA SEQ ID NO: 78, or corresponding mutations in another adenosine deaminase.

In some embodiments, the adenosine deaminase comprises or consists of one, two, three, four, five, six, seven, eight, nine, ten, or eleven mutations selected from H36X, R51X, L84X, A106X, D108X, H123X, S146X, D147X, E155X, I156X, and K157X in ecTadA SEQ ID NO: 78, or a corresponding mutation or mutations in another adenosine deaminase, where X indicates the presence of any amino acid other than the corresponding amino acid in the wild-type adenosine deaminase. In some embodiments, the adenosine deaminase comprises or consists of one, two, three, four, five, six, seven, eight, nine, ten, or eleven mutations selected from H36L, R51L, L84F, A106V, D108N, H123Y, S146C, D147Y, E155V, I156F, and K157N in ecTadA SEQ ID NO: 78, or a corresponding mutation or mutations in another adenosine deaminase. In some embodiments, the adenosine deaminase comprises or consists of a H36L, R51L, L84F, A106V, D108N, H123Y, S146C, D147Y, E155V, I156F, and K157N mutation in ecTadA SEQ ID NO: 78, or corresponding mutations in another adenosine deaminase.

In some embodiments, the adenosine deaminase comprises or consists of one, two, three, four, five, six, seven, eight, nine, ten, eleven, or twelve mutations selected from H36X, P48X, R51X, L84X, A106X, D108X, H123X, S146X, D147X, E155X, I156X, and K157X in ecTadA SEQ ID NO: 78, or a corresponding mutation or mutations in another adenosine deaminase, where X indicates the presence of any amino acid other than the corresponding amino acid in the wild-type adenosine deaminase. In some embodiments, the adenosine deaminase comprises or consists of one, two, three, four, five, six, seven, eight, nine, ten, eleven, or twelve mutations selected from H36L, P48S, R51L, L84F, A106V, D108N, H123Y, S146C, D147Y, E155V, I156F, and K157N in ecTadA SEQ ID NO: 78, or a corresponding mutation or mutations in another adenosine deaminase. In some embodiments, the adenosine deaminase comprises or consists of a H36L, P48S, R51L, L84F, A106V, D108N, H123Y, S146C, D147Y, E155V, I156F, and K157N mutation in ecTadA SEQ ID NO: 78, or corresponding mutations in another adenosine deaminase.

In some embodiments, the adenosine deaminase comprises or consists of one, two, three, four, five, six, seven, eight, nine, ten, eleven, twelve, or thirteen mutations selected from H36X, P48X, R51X, L84X, A106X, D108X, H123X, A142X, S146X, D147X, E155X, I156X, and K157X in ecTadA SEQ ID NO: 78, or a corresponding mutation or mutations in another adenosine deaminase, where X indicates the presence of any amino acid other than the corresponding amino acid in the wild-type adenosine deaminase. In some embodiments, the adenosine deaminase comprises or consists of one, two, three, four, five, six, seven, eight, nine, ten, eleven, twelve, or thirteen mutations selected from H36L, P48S, R51L, L84F, A106V, D108N, H123Y, A142N, S146C, D147Y, E155V, I156F, and K157N in ecTadA SEQ ID NO: 78, or a corresponding mutation or mutations in another adenosine deaminase. In some embodiments, the adenosine deaminase comprises or consists of a H36L, P48S, R51L, L84F, A106V, D108N, H123Y, A142N, S146C, D147Y, E155V, I156F, and K157N mutation in ecTadA SEQ ID NO: 78, or corresponding mutations in another adenosine deaminase.

In some embodiments, the adenosine deaminase comprises or consists of one, two, three, four, five, six, seven, eight, nine, ten, eleven, twelve, thirteen, or fourteen mutations selected from W23X, H36X, P48X, R51X, L84X, A106X, D108X, H123X, A142X, S146X, D147X, E155X, I156X, and K157X in ecTadA SEQ ID NO: 78, or a corresponding mutation or mutations in another adenosine deaminase, where X indicates the presence of any amino acid other than the corresponding amino acid in the wild-type adenosine deaminase. In some embodiments, the adenosine deaminase comprises or consists of one, two, three, four, five, six, seven, eight, nine, ten, eleven, twelve, thirteen, or fourteen mutations selected from W23L, H36L, P48A, R51L, L84F, A106V, D108N, H123Y, A142N, S146C, D147Y, E155V, I156F, and K157N in ecTadA SEQ ID NO: 78 or a corresponding mutation or mutations in another adenosine deaminase. In some embodiments, the adenosine deaminase comprises or consists of a W23L, H36L, P48A, R51L, L84F, A106V, D108N, H123Y, A142N, S146C, D147Y, E155V, I156F, and K157N mutation in ecTadA SEQ ID NO: 78, or corresponding mutations in another adenosine deaminase.

In some embodiments, the adenosine deaminase comprises or consists of one, two, three, four, five, six, seven, eight, nine, ten, eleven, twelve, thirteen, or fourteen mutations selected from W23X, H36X, P48X, R51X, L84X, A106X, D108X, H123X, S146X, D147X, R152X, E155X, I156X, and K157X in ecTadA SEQ ID NO: 78, or a corresponding mutation or mutations in another adenosine deaminase, where X indicates the presence of any amino acid other than the corresponding amino acid in the wild-type adenosine deaminase. In some embodiments, the adenosine deaminase comprises or consists of one, two, three, four, five, six, seven, eight, nine, ten, eleven, twelve, thirteen, or fourteen mutations selected from W23R, H36L, P48A, R51L, L84F, A106V, D108N, H123Y, S146C, D147Y, R152P, E155V, I156F, and K157N in ecTadA SEQ ID NO: 78, or a corresponding mutation or mutations in another adenosine deaminase. In some embodiments, the adenosine deaminase comprises or consists of a W23R, H36L, P48A, R51L, L84F, A106V, D108N, H123Y, S146C, D147Y, R152P, E155V, I156F, and K157N mutation in ecTadA SEQ ID NO: 78, or corresponding mutations in another adenosine deaminase.

In some embodiments, the adenosine deaminase comprises or consists of one, two, three, four, five, six, seven, eight, nine, ten, eleven, twelve, thirteen, fourteen, or fifteen mutations selected from W23X, H36X, P48X, R51X, L84X, A106X, D108X, H123X, A142X, S146X, D147X, R152X, E155X, I156X, and K157X in ecTadA SEQ ID NO: 78, or a corresponding mutation or mutations in another adenosine deaminase, where X indicates the presence of any amino acid other than the corresponding amino acid in the wild-type adenosine deaminase. In some embodiments, the adenosine deaminase comprises or consists of one, two, three, four, five, six, seven, eight, nine, ten, eleven, twelve, thirteen, fourteen, or fifteen mutations selected from W23L, H36L, P48A, R51L, L84F, A106V, D108N, H123Y, A142N, S146C, D147Y, R152P, E155V, I156F, and K157N in ecTadA SEQ ID NO: 78, or a corresponding mutation or mutations in another adenosine deaminase. In some embodiments, the adenosine deaminase comprises or consists of a W23L, H36L, P48A, R51L, L84F, A106V, D108N, H123Y, A142N, S146C, D147Y, R152P, E155V, I156F, and K157N mutation in ecTadA SEQ ID NO: 78, or corresponding mutations in another adenosine deaminase.

In some embodiments, the adenosine deaminase comprises one or more of the mutations provided herein corresponding to ecTadA SEQ ID NO: 78, or one or more of the corresponding mutations in another deaminase. In some embodiments, the adenosine deaminase comprises or consists of a variant of ecTadA SEQ ID NO: 78 provided herein, or the corresponding variant in another adenosine deaminase.

It should be appreciated that the adenosine deaminase (e.g., a first or second adenosine deaminase) may comprise one or more of the mutations provided in any of the adenosine deaminases (e.g., ecTadA adenosine deaminases) provided herein. In some embodiments, the adenosine deaminase comprises the combination of mutations of any of the adenosine deaminases (e.g., ecTadA adenosine deaminases) provided herein. For example, the adenosine deaminase may comprise the mutations W23R, H36L, P48A, R51L, L84F, A106V, D108N, H123Y, S146C, D147Y, R152P, E155V, I156F, and K157N (relative to ecTadA SEQ ID NO: 78), which corresponds to ABE7.10 provided herein. In some embodiments, the adenosine deaminase may comprise the mutations H36L, R51L, L84F, A106V, D108N, H123Y, S146C, D147Y, E155V, I156F, and K157N (relative to ecTadA SEQ ID NO: 78).

In some embodiments, the adenosine deaminase comprises any of the following combination of mutations relative to ecTadA SEQ ID NO: 78, where each mutation of a combination is separated by a “_” and each combination of mutations is between parentheses: (A106V_D108N), (R107C_D108N), (H8Y_D108N_S127S_D147Y_Q154H), (H8Y_R24W_D108N_N127S_D147Y_E155V), (D108N_D147Y_E155V), (H8Y_D108N_S127S), (H8Y_D108N_N127S_D147Y_Q154H), (A106V_D108N_D147Y_E155V), (D108Q_D147Y_E155V), (D108M_D147Y_E155V), (D108L_D147Y_E155V), (D108K_D147Y_E155V), (D108I_D147Y_E155V), (D108F_D147Y_E155V), (A106V_D108N_D147Y), (A106V_D108M_D147Y_E155V), (E59A_A106V_D108N_D147Y_E155V), (E59A cat dead_A106V_D108N_D147Y_E155V), (L84F_A106V_D108N_H123Y_D147Y_E155V_I156Y), (L84F_A106V_D108N_H123Y_D147Y_E155V_I156F), (D103A_D014N), (G22P_D103A_D104N), (G22P_D103A_D104N_S138A), (D103A_D104N_S138A), (R26G_L84F_A106V_R107H_D108N_H123Y_A142N_A143D_D147Y_E155V_I156F), (E25G_R26G_L84F_A106V_R107H_D108N_H123Y_A142N_A143D_D147Y_E155V_I156F), (E25D_R26G_L84F_A106V_R107K_D108N_H123Y_A142N_A143G_D147Y_E155V_I156F), (R26Q_L84F_A106V_D108N_H123Y_A142N_D147Y_E155V_I156F), (E25M_R26G_L84F_A106V_R107P_D108N_H123Y_A142N_A143D_D147Y_E155V_I156F), (R26C_L84F_A106V_R107H_D108N_H123Y_A142N_D147Y_E155V_I156F), (L84F_A106V_D108N_H123Y_A142N_A143L_D147Y_E155V_I156F), (R26G_L84F_A106V_D108N_H123Y_A142N_D147Y_E155V_I156F), (E25A_R26G_L84F_A106V_R107N_D108N_H123Y_A142N_A143E_D147Y_E155V_I156F), (R26G_L84F_A106V_R107H_D108N_H123Y_A142N_A143D_D147Y_E155V_I156F), (A106V_D108N_A142N_D147Y_E155V), (R26G_A106V_D108N_A142N_D147Y_E155V), (E25D_R26G_A106V_R107K_D108N_A142N_A143G_D147Y_E155V), (R26G_A106V_D108N_R107H_A142N_A143D_D147Y_E155V), (E25D_R26G_A106V_D108N_A142N_D147Y_E155V), (A106V_R107K_D108N_A142N_D147Y_E155V), (A106V_D108N_A142N_A143G_D147Y_E155V), (A106V_D108N_A142N_A143L_D147Y_E155V), (H36L_R51L_L84F_A106V_D108N_H123Y_S146C_D147Y_E155V_I156F_K157N), (H36L_P48S_R51L_L84F_A106V_D108N_H123Y_S146C_D147Y_E155V_I156F_K157N), (H36L_P48S_R51L_L84F_A106V_D108N_H123Y_A142N_S146C_D147Y_E155V_I156F_K157N), (W23L_H36L_P48A_R51L_L84F_A106V_D108N_H123Y_A142N_S146C_D147Y_E155V_I156F_K157N), (W23L_H36L_P48A_R51L_L84F_A106V_D108N_H123Y_A142N_S146C_D147Y_R152P_E155V_I156F_K157N), (N37T_P48T_M70L_L84F_A106V_D 108N_H123Y_D147Y_I49V_E155V_I156F), (N37S_L84F_A106V_D108N_H123Y_D147Y_E155V_I156F_K161T), (H36L_L84F_A106V_D108N_H123Y_D147Y_Q154H_E155V_I156F), (N72S_L84F_A106V_D108N_H123Y_S146R_D147Y_E155V_I156F), (H36L_P48L_L84F_A106V_D108N_H123Y_E134G_D147Y_E155V_I156F), (H36L_L84F_A106V_D108N_H123Y_D147Y_E155V_I156F_K157N), (H36L_L84F_A106V_D108N_H123Y_S146C_D147Y_E155V_I156F), (L84F_A106V_D108N_H123Y_S146R_D147Y_E155V_I156F_K161 T), (N37S_R51H_D77G_L84F_A106V_D108N_H123Y_D147Y_E155V_I156F), (R51L_L84F_A106V_D108N_H123Y_D147Y_E155V_I156F_K157N), (D24G_Q71R_L84F_H96L_A106V_D108N_H123Y_D147Y_E155V_I156F_K160E), (H36L_G67V_L84F_A106V_D108N_H123Y_S146T_D147Y_E155V_I156F), (Q71L_L84F_A106V_D108N_H123Y_L137M_A143E_D147Y_E155V_I156F), (E25G_L84F_A106V_D108N_H123Y_D147Y_E155V_I156F_Q159L), (L84F_A91T_F104I_A106V_D108N_H123Y_D147Y_E155V_I156F), (N72D_L84F_A106V_D108N_H123Y_G125A_D147Y_E155V_I156F), (P48S_L84F_S97C_A106V_D108N_H123Y_D147Y_E155V_I156F), (W23G_L84F_A106V_D108N_H123Y_D147Y_E155V_I156F), (D24G_P48L_Q71R_L84F_A106V_D108N_H123Y_D147Y_E155V_I156F_Q159L), (L84F_A106V_D108N_H123Y_A142N_D147Y_E155V_I156F), (H36L_R51L_L84F_A106V_D108N_H123Y_A142N_S146C_D147Y_E155V_I156F_K157N), (N37S_L84F_A106V_D108N_H123Y_A142N_D147Y_E155V_I156F_K161T), (L84F_A106V_D108N_D147Y_E155V_I156F), (R51L_L84F_A106V_D108N_H123Y_S146C_D147Y_E155V_I156F_K157N_K161T), (L84F_A106V_D108N_H123Y_S146C_D147Y_E155V_I156F_K161 T), (L84F_A106V_D108N_H123Y_S146C_D147Y_E155V_I156F_K157N_K160E_K161T), (L84F_A106V_D108N_H123Y_S146C_D147Y_E155V_I156F_K157N_K160E), (R74Q L84F_A106V_D108N_H123Y_D147Y_E155V_I156F), (R74A_L84F_A106V_D108N_H123Y_D147Y_E155V_I156F), (L84F_A106V_D108N_H123Y_D147Y_E155V_I156F), (R74Q_L84F_A106V_D108N_H123Y_D147Y_E155V_I156F), (L84F_R98Q_A106V_D108N_H123Y_D147Y_E155V_I156F), (L84F_A106V_D108N_H123Y_R129Q_D147Y_E155V_I156F), (P48S_L84F_A106V_D108N_H123Y_A142N_D147Y_E155V_I156F), (P48S_A142N), (P48T_I49V_L84F_A106V_D108N_H123Y_A142N_D147Y_E155V_I156F_L157N), (P48T_I49V_A142N), (H36L_P48S_R51L_L84F_A106V_D108N_H123Y_S146C_D147Y_E155V_I156F_K157N), (H36L_P48S_R51L_L84F_A106V_D108N_H123Y_S146C_A142N_D147Y_E155V_I156F_K157N), (H36L_P48T_I49V_R51L_L84F_A106V_D108N_H123Y_S146C_D147Y_E155V_I156F_K157N), (H36L_P48T_I49V_R51L_L84F_A106V_D108N_H123Y_A142N_S146C_D147Y_E155V_I156F_K157N), (H36L_P48A_R51L_L84F_A106V_D108N_H123Y_S146C_D147Y_E155V_I156F_K157N), (H36L_P48A_R51L_L84F_A106V_D108N_H123Y_A142N_S146C_D147Y_E155V_I156F_K157N), (H36L_P48A_R51L_L84F_A106V_D108N_H123Y_S146C_A142N_D147Y_E155V_I156F_K157N), (W23L_H36L_P48A_R51L_L84F_A106V_D108N_H123Y_S146C_D147Y_E155V_I156F_K157N), (W23R_H36L_P48A_R51L_L84F_A106V_D108N_H123Y_S146C_D147Y_E155V_I156F_K157N), (W23L_H36L_P48A_R51L_L84F_A106V_D108N_H123Y_S146R_D147Y_E155V_I156F_K161T), (H36L_P48A_R51L_L84F_A106V_D108N_H123Y_S146C_D147Y_R152H_E155V_I156F_K157N), (H36L_P48A_R51L_L84F_A106V_D108N_H123Y_S146C_D147Y_R152P_E155V_I156F_K157N), (W23L_H36L_P48A_R51L_L84F_A106V_D108N_H123Y_S146C_D147Y_R152P_E155V_I156F_K157N), (W23L_H36L_P48A_R51L_L84F_A106V_D108N_H123Y_A142A_S146C_D147Y_E155 V_I156F_K157N), (W23L_H36L_P48A_R51L_L84F_A106V_D108N_H123Y_A142A_S146C_D147Y_R152P_E155V_I156F_K157N), (W23L_H36L_P48A_R51L_L84F_A106V_D108N_H123Y_S146R_D147Y_E155V_I156F_K161T), (W23R_H36L_P48A_R51L_L84F_A106V_D108N_H123Y_S146C_D147Y_R152P_E155V_I156F_K157N), (H36L_P48A_R51L_L84F_A106V_D108N_H123Y_A142N_S146C_D147Y_R152P_E155V_I156F_K157N).

IV. Cytidine Deaminases (or Cytosine Deaminases)

In some embodiments, the disclosure provides base editors that comprise one or more cytidine deaminase domains. In some aspects, any of the disclosed base editors are capable of deaminating cytidine in a nucleic acid sequence (e.g., genomic DNA). As one example, any of the base editors provided herein may be base editors, (e.g., cytidine base editors).

In some embodiments, the cytidine deaminase is an apolipoprotein B mRNA-editing complex (APOBEC) family deaminase. In some embodiments, the cytidine deaminase is an APOBEC1 deaminase, an APOBEC2 deaminase, an APOBEC3A deaminase, an APOBEC3B deaminase, an APOBEC3C deaminase, an APOBEC3D deaminase, an APOBEC3F deaminase, an APOBEC3G deaminase, an APOBEC3H deaminase, or an APOBEC4 deaminase. In some embodiments, the cytidine deaminase is an activation-induced deaminase (AID). In some embodiments, the deaminase is a Lamprey CDA1 (pmCDA1) deaminase. In some embodiments, the cytidine deaminase is from a human, chimpanzee, gorilla, monkey, cow, dog, rat, or mouse. In some embodiments, the deaminase is from a human. In some embodiments the deaminase is from a rat. In some embodiments, the cytidine deaminase is a human APOBEC1 deaminase. In some embodiments, the cytidine deaminase is pmCDA1. In some embodiments, the deaminase is human APOBEC3G. In some embodiments, the deaminase is a human APOBEC3G variant. In some embodiments, the deaminase is at least 80%, at least 85%, at least 90%, at least 92%, at least 95%, at least 96%, at least 97%, at least 98%, at least 99%, or at least 99.5% identical to any one of the APOBEC amino acid sequences set forth herein.

Some exemplary suitable cytidine deaminases domains that can be fused to Cas9 domains according to aspects of this disclosure are provided below. It should be understood that the disclosure also embraces other cytidine deaminases comprising an amino acid sequence having at least 80%, at least 85%, at least 90%, at least 95%, at least 98%, at least 99%, or at least 99.5% sequence identity to one of the following exemplary cytidine deaminases:

Human AID:
(SEQ ID NO: 92)
MDSLLMNRRKFLYQFKNVRWAKGRRETYLCYVVKR
RDSATSFSLDFGYLRNKNGCHVELLFLRYISDWDL
DPGRCYRVTWFTSWSPCYDCARHVADFLRGNPNLS
LRIFTARLYFCEDRKAEPEGLRRLHRAGVQIAIMT
FKDYFYCWNTFVENHERTFKAWEGLHENSVRLSRQ
LRRILLPLYEVDDLRDAFRTLGL
Mouse AID:
(SEQ ID NO: 93)
MDSLLMKQKKFLYHFKNVRWAKGRHETYLCYVVKR
RDSATSCSLDFGHLRNKSGCHVELLFLRYISDWDL
DPGRCYRVTWFTSWSPCYDCARHVAEFLRWNPNLS
LRIFTARLYFCEDRKAEPEGLRRLHRAGVQIGIMT
FKDYFYCWNTFVENRERTFKAWEGLHENSVRLTRQ
LRRILLPLYEVDDLRDAFRMLGF
Dog AID:
(SEQ ID NO: 94)
MDSLLMKQRKFLYHFKNVRWAKGRHETYLCYVVKRR
DSATSFSLDFGHLRNKSGCHVELLFLRYISDWDLD
PGRCYRVTWFTSWSPCYDCARHVADFLRGYPNLSL
RIFAARLYFCEDRKAEPEGLRRLHRAGVQIAIMTF
KDYFYCWNTFVENREKTFKAWEGLHENSVRLSRQL
RRILLPLYEVDDLRDAFRTLGL
Bovine AID:
(SEQ ID NO: 95)
MDSLLKKQRQFLYQFKNVRWAKGRHETYLCYVVKR
RDSPTSFSLDFGHLRNKAGCHVELLFLRYISDWDL
DPGRCYRVTWFTSWSPCYDCARHVADFLRGYPNLS
LRIFTARLYFCDKERKAEPEGLRRLHRAGVQIAIM
TFKDYFYCWNTFVENHERTFKAWEGLHENSVRLSR
QLRRILLPLYEVDDLRDAFRTLGL
Rat AID:
(SEQ ID NO: 96)
MAVGSKPKAALVGPHWERERIWCFLCSTGLGTQQT
GQTSRWLRPAATQDPVSPPRSLLMKQRKFLYHFKN
VRWAKGRHETYLCYVVKRRDSATSFSLDFGYLRNK
SGCHVELLFLRYISDWDLDPGRCYRVTWFTSWSPC
YDCARHVADFLRGNPNLSLRIFTARLTGWGALPAG
LMSPARPSDYFYCWNTFVENHERTFKAWEGLHENS
VRLSRRLRRILLPLYEVDDLRDAFRTLGL
Mouse APOBEC-3:
(SEQ ID NO: 97)
MGPFCLGCSHRKCYSPIRNLISQETFKFHFKNLGY
AKGRKDTFLCYEVTRKDCDSPVSLHHGVFKNKDNI
HAEICFLYWFHDKVLKVLSPREEFKITWYMSWSPC
FECAEQIVRFLATHHNLSLDIFSSRLYNVQDPETQ
QNLCRLVQEGAQVAAMDLYEFKKCWKKFVDNGGRR
FRPWKRLLTNFRYQDSKLQEILRPCYIPVPSSSSS
TLSNICLTKGLPETRFCVEGRRMDPLSEEEFYSQF
YNQRVKHLCYYHRMKPYLCYQLEQFNGQAPLKGCL
LSEKGKQHAEILFLDKIRSMELSQVTITCYLTWSP
CPNCAWQLAAFKRDRPDLILHIYTSRLYFHWKRPF
QKGLCSLWQSGILVDVMDLPQFTDCWTNFVNPKRP
FWPWKGLEIISRRTQRRLRRIKESWGLQDLVNDFG
NLQLGPPMS
Rat APOBEC-3:
(SEQ ID NO: 98)
MGPFCLGCSHRKCYSPIRNLISQETFKFHFKNLRY
AIDRKDTFLCYEVTRKDCDSPVSLHHGVFKNKDNI
HAEICFLYWFHDKVLKVLSPREEFKITWYMSWSPC
FECAEQVLRFLATHHNLSLDIFSSRLYNIRDPENQ
QNLCRLVQEGAQVAAMDLYEFKKCWKKFVDNGGRR
FRPWKKLLTNFRYQDSKLQEILRPCYIPVPSSSSS
TLSNICLTKGLPETRFCVERRRVHLLSEEEFYSQF
YNQRVKHLCYYHGVKPYLCYQLEQFNGQAPLKGCL
LSEKGKQHAEILFLDKIRSMELSQVIITCYLTWSP
CPNCAWQLAAFKRDRPDLILHIYTSRLYFHWKRPF
QKGLCSLWQSGILVDVMDLPQFTDCWTNFVNPKRP
FWPWKGLEIISRRTQRRLHRIKESWGLQDLVNDFG
NLQLGPPMS
Rhesus macaque APOBEC-3G:
(SEQ ID NO: 99)
MVEPMDPRTFVSNFNNRPILSGLNTVWLCCEVKTK
DPSGPPLDAKIFQGKVYSKAKYHPEMRFLRWFHKW
RQLHHDQEYKVTWYVSWSPCTRCANSVATFLAKDP
KVTLTIFVARLYYFWKPDYQQALRILCQKRGGPHA
TMKIMNYNEFQDCWNKFVDGRGKPFKPRNNLPKHY
TLLQATLGELLRHLMDPGTFTSNFNNKPWVSGQHE
TYLCYKVERLHNDTWVPLNQHRGFLRNQAPNIHGF
PKGRHAELCFLDLIPFWKLDGQQYRVTCFTSWSPC
FSCAQEMAKFISNNEHVSLCIFAARIYDDQGRYQE
GLRALHRDGAKIAMMNYSEFEYCWDTFVDRQGRPF
QPWDGLDEHSQALSGRLRAI
(SEQ ID NO: 100)
Chimpanzee APOBEC-3 G:
MKPHFRNPVERMYQDTFSDNFYNRPILSHRNTVWL
CYEVKTKGPSRPPLDAKIFRGQVYSKLKYHPEMRF
FHWFSKWRKLHRDQEYEVTWYISWSPCTKCTRDVA
TFLAEDPKVTLTIFVARLYYFWDPDYQEALRSLCQ
KRDGPRATMKIMNYDEFQHCWSKFVYSQRELFEPW
NNLPKYYILLHIMLGEILRHSMDPPTFTSNFNNEL
WVRGRHETYLCYEVERLHNDTWVLLNQRRGFLCNQ
APHKHGFLEGRHAELCFLDVIPFWKLDLHQDYRVT
CFTSWSPCFSCAQEMAKFISNNKHVSLCIFAARIY
DDQGRCQEGLRTLAKAGAKISIMTYSEFKHCWDTF
VDHQGCPFQPWDGLEEHSQALSGRLRAILQNQGN
Green monkey APOBEC-3G:
(SEQ ID NO: 101)
MNPQIRNMVEQMEPDIFVYYFNNRPILSGRNTVWL
CYEVKTKDPSGPPLDANIFQGKLYPEAKDHPEMKF
LHWFRKWRQLHRDQEYEVTWYVSWSPCTRCANSVA
TFLAEDPKVTLTIFVARLYYFWKPDYQQALRILCQ
ERGGPHATMKIMNYNEFQHCWNEFVDGQGKPFKPR
KNLPKHYTLLHATLGELLRHVMDPGTFTSNFNNKP
WVSGQRETYLCYKVERSHNDTWVLLNQHRGFLRNQ
APDRHGFPKGRHAELCFLDLIPFWKLDDQQYRVTC
FTSWSPCFSCAQKMAKFISNNKHVSLCIFAARIYD
DQGRCQEGLRTLHRDGAKIAVMNYSEFEYCWDTFV
DRQGRPFQPWDGLDEHSQALSGRLRAI
Human APOBEC-3 G:
(SEQ ID NO: 102)
MKPHFRNTVERMYRDTFSYNFYNRPILSRRNTVWL
CYEVKTKGPSRPPLDAKIFRGQVYSELKYHPEMRF
FHWFSKWRKLHRDQEYEVTWYISWSPCTKCTRDMA
TFLAEDPKVTLTIFVARLYYFWDPDYQEALRSLCQ
KRDGPRATMKIMNYDEFQHCWSKFVYSQRELFEPW
NNLPKYYILLHIMLGEILRHSMDPPTFTFNFNNEP
WVRGRHETYLCYEVERMHNDTWVLLNQRRGFLCNQ
APHKHGFLEGRHAELCFLDVIPFWKLDLDQDYRVT
CFTSWSPCFSCAQEMAKFISKNKHVSLCIFTARIY
DDQGRCQEGLRTLAEAGAKISIMTYSEFKHCWDTF
VDHQGCPFQPWDGLDEHSQDLSGRLRAILQNQEN
Human APOBEC-3F:
(SEQ ID NO: 103)
MKPHFRNTVERMYRDTFSYNFYNRPILSRRNTVWL
CYEVKTKGPSRPRLDAKIFRGQVYSQPEHHAEMCF
LSWFCGNQLPAYKCFQITWFVSWTPCPDCVAKLAE
FLAEHPNVTLTISAARLYYYWERDYRRALCRLSQA
GARVKIMDDEEFAYCWENFVYSEGQPFMPWYKFDD
NYAFLHRTLKEILRNPMEAMYPHIFYFHFKNLRKA
YGRNESWLCFTMEVVKHHSPVSWKRGVFRNQVDPE
THCHAERCFLSWFCDDILSPNTNYEVTWYTSWSPC
PECAGEVAEFLARHSNVNLTIFTARLYYFWDTDYQ
EGLRSLSQEGASVEIMGYKDFKYCWENFVYNDDEP
FKPWKGLKYNFLFLDSKLQEILE
Human APOBEC-3B:
(SEQ ID NO: 104)
MNPQIRNPMERMYRDTFYDNFENEPILYGRSYTWL
CYEVKIKRGRSNLLWDTGVFRGQVYFKPQYHAEMC
FLSWFCGNQLPAYKCFQITWFVSWTPCPDCVAKLA
EFLSEHPNVTLTISAARLYYYWERDYRRALCRLSQ
AGARVTIMDYEEFAYCWENFVYNEGQQFMPWYKFD
ENYAFLHRTLKEILRYLMDPDTFTFNFNNDPLVLR
RRQTYLCYEVERLDNGTWVLMDQHMGFLCNEAKNL
LCGFYGRHAELRFLDLVPSLQLDPAQIYRVTWFIS
WSPCFSWGCAGEVRAFLQENTHVRLRIFAARIYDY
DPLYKEALQMLRDAGAQVSIMTYDEFEYCWDTFVY
RQGCPFQPWDGLEEHSQALSGRLRAILQNQGN
Rat APOBEC-3B:
(SEQ ID NO: 105)
MQPQGLGPNAGMGPVCLGCSHRRPYSPIRNPLKKL
YQQTFYFHFKNVRYAWGRKNNFLCYEVNGMDCALP
VPLRQGVFRKQGHIHAELCFIYWFHDKVLRVLSPM
EEFKVTWYMSWSPCSKCAEQVARFLAAHRNLSLAI
FSSRLYYYLRNPNYQQKLCRLIQEGVHVAAMDLPE
FKKCWNKFVDNDGQPFRPWMRLRINFSFYDCKLQE
IFSRMNLLREDVFYLQFNNSHRVKPVQNRYYRRKS
YLCYQLERANGQEPLKGYLLYKKGEQHVEILFLEK
MRSMELSQVRITCYLTWSPCPNCARQLAAFKKDHP
DLILRIYTSRLYFYWRKKFQKGLCTLWRSGIHVDV
MDLPQFADCWTNFVNPQRPFRPWNELEKNSWRIQR
RLRRIKESWGL
Bovine APOBEC-3B:
(SEQ ID NO: 106)
DGWEVAFRSGTVLKAGVLGVSMTEGWAGSGHPGQG
ACVWTPGTRNTMNLLREVLFKQQFGNQPRVPAPYY
RRKTYLCYQLKQRNDLTLDRGCFRNKKQRHAEIRF
IDKINSLDLNPSQSYKIICYITWSPCPNCANELVN
FITRNNHLKLEIFASRLYFHWIKSFKMGLQDLQNA
GISVAVMTHTEFEDCWEQFVDNQSRPFQPWDKLEQ
YSASIRRRLQRILTAPI
Chimpanzee APOBEC-3B:
(SEQ ID NO: 107)
MNPQIRNPMEWMYQRTFYYNFENEPILYGRSYTWL
CYEVKIRRGHSNLLWDTGVFRGQMYSQPEHHAEMC
FLSWFCGNQLSAYKCFQITWFVSWTPCPDCVAKLA
KFLAEHPNVTLTISAARLYYYWERDYRRALCRLSQ
AGARVKIMDDEEFAYCWENFVYNEGQPFMPWYKFD
DNYAFLHRTLKEIIRHLMDPDTFTFNFNNDPLVLR
RHQTYLCYEVERLDNGTWVLMDQHMGFLCNEAKNL
LCGFYGRHAELRFLDLVPSLQLDPAQIYRVTWFIS
WSPCFSWGCAGQVRAFLQENTHVRLRIFAARIYDY
DPLYKEALQMLRDAGAQVSIMTYDEFEYCWDTFVY
RQGCPFQPWDGLEEHSQALSGRLRAILQVRASSLC
MVPHRPPPPPQSPGPCLPLCSEPPLGSLLPTGRPA
PSLPFLLTASFSFPPPASLPPLPSLSLSPGHLPVP
SFHSLTSCSIQPPCSSRIRETEGWASVSKEGRDLG
Human APOBEC-3C:
(SEQ ID NO: 108)
MNPQIRNPMKAMYPGTFYFQFKNLWEANDRNETWL
CFTVEGIKRRSVVSWKTGVFRNQVDSETHCHAERC
FLSWFCDDILSPNTKYQVTWYTSWSPCPDCAGEVA
EFLARHSNVNLTIFTARLYYFQYPCYQEGLRSLSQ
EGVAVEIMDYEDFKYCWENFVYNDNEPFKPWKGLK
TNFRLLKRRLRESLQ
Gorilla APOBEC3C:
(SEQ ID NO: 109)
MNPQIRNPMKAMYPGTFYFQFKNLWEANDRNETWL
CFTVEGIKRRSVVSWKTGVFRNQVDSETHCHAERC
FLSWFCDDILSPNTNYQVTWYTSWSPCPECAGEVA
EFLARHSNVNLTIFTARLYYFQDTDYQEGLRSLSQ
EGVAVKIMDYKDFKYCWENFVYNDDEPFKPWKGLK
YNFRFLKRRLQEILE
Human APOBEC-3 A:
(SEQ ID NO: 110)
MEASPASGPRHLMDPHIFTSNFNNGIGRHKTYLCY
EVERLDNGTSVKMDQHRGFLHNQAKNLLCGFYGRH
AELRFLDLVPSLQLDPAQIYRVTWFISWSPCFSWG
CAGEVRAFLQENTHVRLRIFAARIYDYDPLYKEAL
QMLRDAGAQVSIMTYDEFKHCWDTFVDHQGCPFQP
WDGLDEHSQALSGRLRAILQNQGN
Rhesus macaque APOBEC-3 A:
(SEQ ID NO: 111)
MDGSPASRPRHLMDPNTFTFNFNNDLSVRGRHQTY
LCYEVERLDNGTWVPMDERRGFLCNKAKNVPCGDY
GCHVELRFLCEVPSWQLDPAQTYRVTWFISWSPCF
RRGCAGQVRVFLQENKHVRLRIFAARIYDYDPLYQ
EALRTLRDAGAQVSIMTYEEFKHCWDTFVDRQGRP
FQPWDGLDEHSQALSGRLRAILQNQGN
Bovine APOBEC-3 A:
(SEQ ID NO: 112)
MDEYTFTENFNNQGWPSKTYLCYEMERLDGDATIP
LDEYKGFVRNKGLDQPEKPCHAELYFLGKIHSWNL
DRNQHYRLTCFISWSPCYDCAQKLTTFLKENHHIS
LHILASRIYTHNRFGCHQSGLCELQAAGARITIMT
FEDFKHCWETFVDHKGKPFQPWEGLNVKSQALCTE
LQAILKTQQN
Human APOBEC-3H:
(SEQ ID NO: 113)
MALLTAETFRLQFNNKRRLRRPYYPRKALLCYQLT
PQNGSTPTRGYFENKKKCHAEICFINEIKSMGLDE
TQCYQVTCYLTWSPCSSCAWELVDFIKAHDHLNLG
IFASRLYYHWCKPQQKGLRLLCGSQVPVEVMGFPK
FADCWENFVDHEKPLSFNPYKMLEELDKNSRAIKR
RLERIKIPGVRAQGRYMDILCDAEV
Rhesus macaque APOBEC-3H:
(SEQ ID NO: 114)
MALLTAKTFSLQFNNKRRVNKPYYPRKALLCYQLT
PQNGSTPTRGHLKNKKKDHAEIRFINKIKSMGLDE
TQCYQVTCYLTWSPCPSCAGELVDFIKAHRHLNLR
IFASRLYYHWRPNYQEGLLLLCGSQVPVEVMGLPE
FTDCWENFVDHKEPPSFNPSEKLEELDKNSQAIKR
RLERIKSRSVDVLENGLRSLQLGPVTPSSSIRNSR
Human APOBEC-3D:
(SEQ ID NO: 115)
MNPQIRNPMERMYRDTFYDNFENEPILYGRSYTWL
CYEVKIKRGRSNLLWDTGVFRGPVLPKRQSNHRQE
VYFRFENHAEMCFLSWFCGNRLPANRRFQITWFVS
WNPCLPCVVKVTKFLAEHPNVTLTISAARLYYYRD
RDWRWVLLRLHKAGARVKIMDYEDFAYCWENFVCN
EGQPFMPWYKFDDNYASLHRTLKEILRNPMEAMYP
HIFYFHFKNLLKACGRNESWLCFTMEVTKHHSAVF
RKRGVFRNQVDPETHCHAERCFLSWFCDDILSPNT
NYEVTWYTSWSPCPECAGEVAEFLARHSNVNLTIF
TARLCYFWDTDYQEGLCSLSQEGASVKIMGYKDFV
SCWKNFVYSDDEPFKPWKGLQTNFRLLKRRLREIL
Q
Human APOBEC-1:
(SEQ ID NO: 116)
MTSEKGPSTGDPTLRRRIEPWEFDVFYDPRELRKE
ACLLYEIKWGMSRKIWRSSGKNTTNHVEVNFIKKF
TSERDFHPSMSCSITWFLSWSPCWECSQAIREFLS
RHPGVTLVIYVARLFWHMDQQNRQGLRDLVNSGVT
IQIMRASEYYHCWRNFVNYPPGDEAHWPQYPPLWM
MLYALELHCIILSLPPCLKISRRWQNHLTFFRLHL
QNCHYQTIPPHILLATGLIHPSVAWR
Mouse APOBEC-1:
(SEQ ID NO: 117)
MSSETGPVAVDPTLRRRIEPHEFEVFFDPRELRKE
TCLLYEINWGGRHSVWRHTSQNTSNHVEVNFLEKF
TTERYFRPNTRCSITWFLSWSPCGECSRAITEFLS
RHPYVTLFIYIARLYHHTDQRNRQGLRDLISSGVT
IQIMTEQEYCYCWRNFVNYPPSNEAYWPRYPHLWV
KLYVLELYCIILGLPPCLKILRRKQPQLTFFTITL
QTCHYQRIPPHLLWATGLK
Rat APOBEC-1:
(SEQ ID NO: 118)
MSSETGPVAVDPTLRRRIEPHEFEVFFDPRELRKE
TCLLYEINWGGRHSIWRHTSQNTNKHVEVNFIEKF
TTERYFCPNTRCSITWFLSWSPCGECSRAITEFLS
RYPHVTLFIYIARLYHHADPRNRQGLRDLISSGVT
IQIMTEQESGYCWRNFVNYSPSNEAHWPRYPHLWV
RLYVLELYCIILGLPPCLNILRRKQPQLTFFTIAL
QSCHYQRLPPHILWATGLK
Human APOBEC-2:
(SEQ ID NO: 119)
MAQKEEAAVATEAASQNGEDLENLDDPEKLKELIE
LPPFEIVTGERLPANFFKFQFRNVEYSSGRNKTFL
CYVVEAQGKGGQVQASRGYLEDEHAAAHAEEAFFN
TILPAFDPALRYNVTWYVSSSPCAACADRIIKTLS
KTKNLRLLILVGRLFMWEEPEIQAALKKLKEAGCK
LRIMKPQDFEYVWQNFVEQEEGESKAFQPWEDIQE
NFLYYEEKLADILK
Mouse APOBEC-2:
(SEQ ID NO: 120)
MAQKEEAAEAAAPASQNGDDLENLEDPEKLKELID
LPPFEIVTGVRLPVNFFKFQFRNVEYSSGRNKTFL
CYVVEVQSKGGQAQATQGYLEDEHAGAHAEEAFFN
TILPAFDPALKYNVTWYVSSSPCAACADRILKTLS
KTKNLRLLILVSRLFMWEEPEVQAALKKLKEAGCK
LRIMKPQDFEYIWQNFVEQEEGESKAFEPWEDIQE
NFLYYEEKLADILK
Rat APOBEC-2:
(SEQ ID NO: 121)
MAQKEEAAEAAAPASQNGDDLENLEDPEKLKELID
LPPFEIVTGVRLPVNFFKFQFRNVEYSSGRNKTFL
CYVVEAQSKGGQVQATQGYLEDEHAGAHAEEAFFN
TILPAFDPALKYNVTWYVSSSPCAACADRILKTLS
KTKNLRLLILVSRLFMWEEPEVQAALKKLKEAGCK
LRIMKPQDFEYLWQNFVEQEEGESKAFEPWEDIQE
NFLYYEEKLADILK
Bovine APOBEC-2:
(SEQ ID NO: 122)
MAQKEEAAAAAEPASQNGEEVENLEDPEKLKELIE
LPPFEIVTGERLPAHYFKFQFRNVEYSSGRNKTFL
CYVVEAQSKGGQVQASRGYLEDEHATNHAEEAFFN
SIMPTFDPALRYMVTWYVSSSPCAACADRIVKTLN
KTKNLRLLILVGRLFMWEEPEIQAALRKLKEAGCR
LRIMKPQDFEYIWQNFVEQEEGESKAFEPWEDIQE
NFLYYEEKLADILK
Petromyzon marinus CD Al (pmCDAl):
(SEQ ID NO: 123)
MTDAEYVRIHEKLDIYTFKKQFFNNKKSVSHRCYV
LFELKRRGERRACFWGYAVNKPQSGTERGIHAEIF
SIRKVEEYLRDNPGQFTINWYSSWSPCADCAEKIL
EWYNQELRGNGHTLKIWACKLYYEKNARNQIGLWN
LRDNGVGLNVMVSEHYQCCRKIFIQSSHNQLNENR
WLEKTLKRAEKRRSELSIMIQVKILHTTKSPAV
Human APOBEC3G D316R D317R:
(SEQ ID NO: 124)
MKPHFRNTVERMYRDTFSYNFYNRPILSRRNTVWL
CYEVKTKGPSRPPLDAKIFRGQVYSELKYHPEMRF
FHWFSKWRKLHRDQEYEVTWYISWSPCTKCTRDMA
TFLAEDPKVTLTIFVARLYYFWDPDYQEALRSLCQ
KRDGPRATMKIMNYDEFQHCWSKFVYSQRELFEPW
NNLPKYYILLHIMLGEILRHSMDPPTFTFNFNNEP
WVRGRHETYLCYEVERMHNDTWVLLNQRRGFLCNQ
APHKHGFLEGRHAELCFLDVIPFWKLDLDQDYRVT
CFTSWSPCFSCAQEMAKFISKNKHVSLCIFTARIY
RRQGRCQEGLRTLAEAGAKISIMTYSEFKHCWDTF
VDHQGCPFQPWDGLDEHSQDLSGRLRAILQNQEN
Human APOBEC3G chain A:
(SEQ ID NO: 125)
MDPPTFTFNFNNEPWVRGRHETYLCYEVERMHNDT
WVLLNQRRGFLCNQAPHKHGFLEGRHAELCFLDVI
PFWKLDLDQDYRVTCFTSWSPCFSCAQEMAKFISK
NKHVSLCIFTARIYDDQGRCQEGLRTLAEAGAKIS
IMTYSEFKHCWDTFVDHQGCPFQPWDGLDEHSQDL
SGRLRAILQ
Human APOBEC3G chain A D120R D121R:
(SEQ ID NO: 126)
MDPPTFTFNFNNEPWVRGRHETYLCYEVERMHNDT
WVLLNQRRGFLCNQAPHKHGFLEGRHAELCFLDVI
PFWKLDLDQDYRVTCFTSWSPCFSCAQEMAKFISK
NKHVSLCIFTARIYRRQGRCQEGLRTLAEAGAKIS
IMTYSEFKHCWDTFVDHQGCPFQPWDGLDEHSQDL
SGRLRAILQ

Any of the aforementioned DNA effector domains may be subjected to a continuous evolution process (e.g., PACE) or may be otherwise further evolved using a mutagenesis methodology known in the art.

In some embodiments, the cytidine deaminase is an apolipoprotein B mRNA-editing complex (APOBEC) family deaminase. In some embodiments, the deaminase is an APOBEC1 deaminase. In some embodiments, the deaminase is an APOBEC2 deaminase. In some embodiments, the deaminase is an APOBEC3 deaminase. In some embodiments, the deaminase is an APOBEC3A deaminase. In some embodiments, the deaminase is an APOBEC3B deaminase. In some embodiments, the deaminase is an APOBEC3C deaminase. In some embodiments, the deaminase is an APOBEC3D deaminase. In some embodiments, the deaminase is an APOBEC3E deaminase. In some embodiments, the deaminase is an APOBEC3F deaminase. In some embodiments, the deaminase is an APOBEC3G deaminase. In some embodiments, the deaminase is an APOBEC3H deaminase. In some embodiments, the deaminase is an APOBEC4 deaminase. In some embodiments, the deaminase is an activation-induced deaminase (AID). In some embodiments, the deaminase is a vertebrate deaminase. In some embodiments, the deaminase is an invertebrate deaminase. In some embodiments, the deaminase is a human, chimpanzee, gorilla, monkey, cow, dog, rat, or mouse deaminase. In some embodiments, the deaminase is a human deaminase. In some embodiments, the deaminase is a rat deaminase, e.g., rAPOBEC1.

Some aspects of the disclosure are based on the recognition that modulating the deaminase domain catalytic activity of any of the fusion proteins provided herein, for example by making point mutations in the deaminase domain, affect the processivity of the fusion proteins (e.g., base editors). For example, mutations that reduce, but do not eliminate, the catalytic activity of a deaminase domain within a base editing fusion protein can make it less likely that the deaminase domain will catalyze the deamination of a residue adjacent to a target residue, thereby narrowing the deamination window. The ability to narrow the deamination window may prevent unwanted deamination of residues adjacent of specific target residues, which may decrease or prevent off-target effects.

In some embodiments, any of the fusion proteins provided herein comprise a deaminase domain (e.g., a cytidine deaminase domain) that has reduced catalytic deaminase activity. In some embodiments, any of the fusion proteins provided herein comprise a deaminase domain (e.g., a cytidine deaminase domain) that has a reduced catalytic deaminase activity as compared to an appropriate control. For example, the appropriate control may be the deaminase activity of the deaminase prior to introducing one or more mutations into the deaminase. In other embodiments, the appropriate control may be a wild-type deaminase. In some embodiments, the appropriate control is a wild-type apolipoprotein B mRNA-editing complex (APOBEC) family deaminase. In some embodiments, the appropriate control is an APOBEC1 deaminase, an APOBEC2 deaminase, an APOBEC3A deaminase, an APOBEC3B deaminase, an APOBEC3C deaminase, an APOBEC3D deaminase, an APOBEC3F deaminase, an APOBEC3G deaminase, or an APOBEC3H deaminase. In some embodiments, the appropriate control is an activation induced deaminase (AID). In some embodiments, the appropriate control is a cytidine deaminase 1 from Petromyzon marinus (pmCDA1). In some embodiments, the deaminase domain may be a deaminase domain that has at least 1%, at least 5%, at least 15%, at least 20%, at least 25%, at least 30%, at least 40%, at least 50%, at least 60%, at least 70%, at least 80%, at least 90%, or at least 95% less catalytic deaminase activity as compared to an appropriate control.

The apolipoprotein B mRNA-editing complex (APOBEC) family of cytidine deaminase enzymes encompasses eleven proteins that serve to initiate mutagenesis in a controlled and beneficial manner. One family member, activation-induced cytidine deaminase (AID), is responsible for the maturation of antibodies by converting cytosines in ssDNA to uracils in a transcription-dependent, strand-biased fashion. The apolipoprotein B editing complex 3 (APOBEC3) enzyme provides protection to human cells against a certain HIV-1 strain via the deamination of cytosines in reverse-transcribed viral ssDNA. These proteins all require a Zn2+-coordinating motif (His-X-Glu-X23-26-Pro-Cys-X2-4-Cys; (SEQ ID NO: 402) and bound water molecule for catalytic activity. The Glu residue acts to activate the water molecule to a zinc hydroxide for nucleophilic attack in the deamination reaction. Each family member preferentially deaminates at its own particular “hotspot”, ranging from WRC (W is A or T, R is A or G) for hAID, to TTC for hAPOBEC3F. A recent crystal structure of the catalytic domain of APOBEC3G revealed a secondary structure comprised of a five-stranded β-sheet core flanked by six α-helices, which is believed to be conserved across the entire family. The active center loops have been shown to be responsible for both ssDNA binding and in determining “hotspot” identity. Overexpression of these enzymes has been linked to genomic instability and cancer, thus highlighting the importance of sequence-specific targeting.

Some aspects of this disclosure relate to the recognition that the activity of cytidine deaminase enzymes such as APOBEC enzymes can be directed to a specific site in genomic DNA. Without wishing to be bound by any particular theory, advantages of using Cas9 as a recognition agent include (1) the sequence specificity of Cas9 can be easily altered by simply changing the sgRNA sequence; and (2) Cas9 binds to its target sequence by denaturing the dsDNA, resulting in a stretch of DNA that is single-stranded and therefore a viable substrate for the deaminase. It should be understood that other catalytic domains, or catalytic domains from other deaminases, can also be used to generate fusion proteins with Cas9, and that the disclosure is not limited in this regard.

Some aspects of this disclosure are based on the recognition that Cas9:deaminase fusion proteins can efficiently deaminate nucleotides. In view of the results provided herein regarding the nucleotides that can be targeted by Cas9:deaminase fusion proteins, a person of skill in the art will be able to design suitable guide RNAs to target the fusion proteins to a target sequence that comprises a nucleotide to be deaminated.

In certain embodiments, the reference cytidine deaminase domain comprises a “FERNY” polypeptide having an amino acid sequence according to SEQ ID NO: 127 or an amino acid sequence that is at least 60%, 65%, 70%, 75%, 80%, 85%, 90%, 95, 98%, 99%, or 99.5% identical to SEQ ID NO: 127, as follows:

(SEQ ID NO: 127)
MFERNYDPRELRKETYLLYEIKWGKSGKLWRHWCQ
NNRTQHAEVYFLENIFNARRFNPSTHCSITWYLSW
SPCAECSQKIVDFLKEHPNVNLEIYVARLYYHEDE
RNRQGLRDLVNSGVTIRIMDLPDYNYCWKTFVSDQ
GGDEDYWPGHFAPWIKQYSLKL

In certain other embodiment, the evolved cytidine deaminase domain (i.e., as a result of the continuous evolution process described herein) comprises a “evoFERNY” polypeptide having an amino acid sequence according to SEQ ID NO: 128 or an amino acid sequence that is at least 60%, 65%, 70%, 75%, 80%, 85%, 90%, 95, 98%, 99%, or 99.5% identical to SEQ ID NO: 128, comprising an H102P and D104N substitutions, as follows:

(SEQ ID NO: 128)
MFERNYDPRELRKETYLLYEIKWGKSGKLWRHWCQ
NNRTQHAEVYFLENIFNARRFNPSTHCSITWYLSW
SPCAECSQKIVDFLKEHPNVNLEIYVARLYYPENE
RNRQGLRDLVNSGVTIRIMDLPDYNYCWKTFVSDQ
GGDEDYWPGHFAPWIKQYSLKL

In other embodiments, the reference cytidine deaminase domain comprises a “Rat APOBEC-1” polypeptide having an amino acid sequence according to SEQ ID NO: 129 or an amino acid sequence that is at least 60%, 65%, 70%, 75%, 80%, 85%, 90%, 95, 98%, 99%, or 99.5% identical to SEQ ID NO: 129, as follows:

(SEQ ID NO: 129)
MSSETGPVAVDPTLRRRIEPHEFEVFFDPRELRKE
TCLLYEINWGGRHSIWRHTSQNTNKHVEVNFIEKF
TTERYFCPNTRCSITWFLSWSPCGECSRAITEFLS
RYPHVTLFIYIARLYHHADPRNRQGLRDLISSGVT
IQIMTEQESGYCWRNFVNYSPSNEAHWPRYPHLWV
RLYVLELYCIILGLPPCLNILRRKQPQLTFFTIAL
QSCHYQRLPPHILWATGLK

In certain other embodiment, the evolved cytidine deaminase domain (i.e., as a result of the continuous evolution process described herein) comprises a “evoAPOBEC” polypeptide having an amino acid sequence according to SEQ ID NO: 130 or an amino acid sequence that is at least 60%, 65%, 70%, 75%, 80%, 85%, 90%, 95, 98%, 99%, or 99.5% identical to SEQ ID NO: 130, and comprising substitutions E4K; H109N; H122L; D124N; R154H; A165S; P201S; F205S, as follows:

(SEQ ID NO: 130)
MSSKTGPVAVDPTLRRRIEPHEFEVFFDPRELRKE
TCLLYEINWGGRHSIWRHTSQNTNKHVEVNFIEKF
TTERYFCPNTRCSITWFLSWSPCGECSRAITEFLS
RYPNVTLFIYIARLYHLANPRNRQGLRDLISSGVT
IQIMTEQESGYCWHNFVNYSPSNESHWPRYPHLWV
RLYVLELYCIILGLPPCLNILRRKQSQLTSFTIAL
QSCHYQRLPPHILWATGLK

In still other embodiments, the reference cytidine deaminase domain comprises a “Petromyzon marinus CDA1 (pmCDA1)” polypeptide having an amino acid sequence according to SEQ ID NO: 131 or an amino acid sequence that is at least 60%, 65%, 70%, 75%, 80%, 85%, 90%, 95, 98%, 99%, or 99.5% identical to SEQ ID NO: 131, as follows:

(SEQ ID NO: 131)
MTDAEYVRIHEKLDIYTFKKQFFNNKKSVSHRCYV
LFELKRRGERRACFWGYAVNKPQSGTERGIHAEIF
SIRKVEEYLRDNPGQFTINWYSSWSPCADCAEKIL
EWYNQELRGNGHTLKIWACKLYYEKNARNQIGLWN
LRDNGVGLNVMVSEHYQCCRKIFIQSSHNQLNENR
WLEKTLKRAEKRRSELSIMIQVKILHTTKSPAV

In other embodiment, the evolved cytidine deaminase domain (i.e., as a result of the continuous evolution process described herein) comprises a “evoCDA” polypeptide having an amino acid sequence according to SEQ ID NO: 132 or an amino acid sequence that is at least 60%, 65%, 70%, 75%, 80%, 85%, 90%, 95, 98%, 99%, or 99.5% identical to SEQ ID NO: 132 and comprising substitutions F23S; A123V; I195F, as follows:

(SEQ ID NO: 132)
MTDAEYVRIHEKLDIYTFKKQFSNNKKSVSHRCYV
LFELKRRGERRACFWGYAVNKPQSGTERGIHAEIF
SIRKVEEYLRDNPGQFTINWYSSWSPCADCAEKIL
EWYNQELRGNGHTLKIWVCKLYYEKNARNQIGLWN
LRDNGVGLNVMVSEHYQCCRKIFIQSSHNQLNENR
WLEKTLKRAEKRRSELSIMFQVKILHTTKSPAV

In yet other embodiments, the reference cytidine deaminase domain comprises a “Anc689 APOBEC” polypeptide having an amino acid sequence according to SEQ ID NO: 133 or an amino acid sequence that is at least 60%, 65%, 70%, 75%, 80%, 85%, 90%, 95, 98%, 99%, or 99.5% identical to SEQ ID NO: 133, as follows:

(SEQ ID NO: 133)
MSSETGPVAVDPTLRRRIEPHEFEVFFDPRELRKE
TCLLYEIKWGTSHKIWRHSSKNTTKHVEVNFIEKF
TSERHFCPSTSCSITWFLSWSPCGECSKAITEFLS
QHPNVTLVIYVARLYHHMDQQNRQGLRDLVNSGVT
IQIMTAPEYDYCWRNFVNYPPGKEAHWPRYPPLWM
KLYALELHAGILGLPPCLNILRRKQPQLTFFTIAL
QSCHYQRLPPHILWATGLK

In other embodiments, the evolved cytidine deaminase domain (i.e., as a result of the continuous evolution process described herein) comprises a “evoAnc689 APOBEC” polypeptide having an amino acid sequence according to SEQ ID NO: 134 or an amino acid sequence that is at least 60%, 65%, 70%, 75%, 80%, 85%, 90%, 95, 98%, 99%, or 99.5% identical to SEQ ID NO: 134 and comprising substitutions E4K; H122L; D124N; R154H; A165S; P201S; F205S, as follows:

(SEQ ID NO: 134)
MSSKTGPVAVDPTLRRRIEPHEFEVFFDPRELRKE
TCLLYEIKWGTSHKIWRHSSKNTTKHVEVNFIEKF
TSERHFCPSTSCSITWFLSWSPCGECSKAITEFLS
QHPNVTLVIYVARLYHLMNQQNRQGLRDLVNSGVT
IQIMTAPEYDYCWHNFVNYPPGKESHWPRYPPLWM
KLYALELHAGILGLPPCLNILRRKQSQLTSFTIAL
QSCHYQRLPPHILWATGLK

In some aspects, the specification provides evolved cytidine deaminases which are used to construct base editors that have improved properties. For example, evolved cytidine deaminases, such as those provided herein, are capable of improving base editing efficiency and/or improving the ability of base editors to more efficiently edit bases regardless of the surrounding sequence. For example, in some aspects the disclosure provides evolved APOBEC deaminases (e.g., evolved rAPOBEC1) with improved base editing efficiency in the context of a 5′-G-3′ when it is 5′ to a target base (e.g., C). In some embodiments, the disclosure provides base editors comprising any of the evolved cytidine deaminases provided herein. It should be appreciated that any of the evolved cydidine deaminases provided herein may be used as a deaminase in a base editor protein, such as any of the base editors provided herein. It should also be appreciated that the disclosure contemplates cytidine deaminases having any of the mutations provided herein, for example any of the mutations described in the Examples section.

V. Other Functional Domains

In various embodiments, the base editors and their various components may comprise additional functional moeities, such as, but not limited to, linkers, uracil glycosylase inhibitors, nuclear localization signals, split-intein sequences (to join split proteins, such as split napDNAbps, split adenine deaminases, split cytidine deaminases, split CBEs, or split ABEs), and RNA-protein recruitment domains (such as, MS2 tagging system).

(1) Linkers

In certain embodiments, linkers may be used to link any of the protein or protein domains described herein (e.g., a deaminase domain and a Cas9 domain). The linker may be as simple as a covalent bond, or it may be a polymeric linker many atoms in length. In certain embodiments, the linker is a polypeptide or based on amino acids. In other embodiments, the linker is not peptide-like. In certain embodiments, the linker is a covalent bond (e.g., a carbon-carbon bond, disulfide bond, carbon-heteroatom bond, etc.). In certain embodiments, the linker is a carbon-nitrogen bond of an amide linkage. In certain embodiments, the linker is a cyclic or acyclic, substituted or unsubstituted, branched or unbranched aliphatic or heteroaliphatic linker. In certain embodiments, the linker is polymeric (e.g., polyethylene, polyethylene glycol, polyamide, polyester, etc.). In certain embodiments, the linker comprises a monomer, dimer, or polymer of aminoalkanoic acid. In certain embodiments, the linker comprises an aminoalkanoic acid (e.g., glycine, ethanoic acid, alanine, beta-alanine, 3-aminopropanoic acid, 4-aminobutanoic acid, 5-pentanoic acid, etc.). In certain embodiments, the linker comprises a monomer, dimer, or polymer of aminohexanoic acid (Ahx). In certain embodiments, the linker is based on a carbocyclic moiety (e.g., cyclopentane, cyclohexane). In other embodiments, the linker comprises a polyethylene glycol moiety (PEG). In other embodiments, the linker comprises amino acids. In certain embodiments, the linker comprises a peptide. In certain embodiments, the linker comprises an aryl or heteroaryl moiety. In certain embodiments, the linker is based on a phenyl ring. The linker may include functionalized moieties to facilitate attachment of a nucleophile (e.g., thiol, amino) from the peptide to the linker. Any electrophile may be used as part of the linker. Exemplary electrophiles include, but are not limited to, activated esters, activated amides, Michael acceptors, alkyl halides, aryl halides, acyl halides, and isothiocyanates.

In some embodiments, the linker is an amino acid or a plurality of amino acids (e.g., a peptide or protein). In some embodiments, the linker is a bond e.g., a covalent bond), an organic molecule, group, polymer, or chemical moiety. In some embodiments, the linker is 5-100 amino acids in length, for example, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35-40, 40-45, 45-50, 50-60, 60-70, 70-80, 80-90, 90-100, 100-110, 110-120, 120-130, 130-140, 140-150, or 150-200 amino acids in length. Longer or shorter linkers are also contemplated. In some embodiments, a linker comprises the amino acid sequence SGSETPGTSESATPES (SEQ ID NO: 143), which may also be referred to as the XTEN linker. In some embodiments, the linker is 32 amino acids in length. In some embodiments, the linker comprises the amino acid sequence (SGGS)₂—SGSETPGTSESATPES-(SGGS)₂ (SEQ ID NO: 144), which may also be referred to as (SGGS)₂—XTEN-(SGGS)₂ (SEQ ID NO: 144). In some embodiments, the linker comprises the amino acid sequence, wherein n is 0, 1, 2, 3, 4, 5, 6, 7, 8, 9, or 10. In some embodiments, a linker comprises the amino acid sequence SGGS (SEQ ID NO: 138). In some embodiments, a linker comprises (SGGS)ₙ (SEQ ID NO: 139), (GGGS)ₙ (SEQ ID NO: 140), (GGGGS)ₙ (SEQ ID NO: 141), (G)ₙ (SEQ ID NO: 135), (EAAAK)ₙ (SEQ ID NO: 142), (SGGS)ₙ-SGSETPGTSESATPES-(SGGS)ₙ (SEQ ID NO: 145), (GGS)ₙ (SEQ ID NO: 137), SGSETPGTSESATPES (SEQ ID NO: 143), or (XP)ₙ (SEQ ID NO: 136) motif, or a combination of any of these, wherein n is independently an integer between 1 and 30, and wherein X is any amino acid. In some embodiments, n is 1, 2, 3, 4, 5, 6, 7, 8, 9, 10, 11, 12, 13, 14, or 15. In some embodiments, a linker comprises SGSETPGTSESATPES (SEQ ID NO: 143), and SGGS (SEQ ID NO: 138). In some embodiments, a linker comprises SGGSSGSETPGTSESATPESSGGS (SEQ ID NO: 145). In some embodiments, a linker comprises SGGSSGGSSGSETPGTSESATPESSGGSSGGS (SEQ ID NO: 147). In some embodiments, a linker comprises GGSGGSPGSPAGSPTSTEEGTSESATPESGPGTSTEPSEGSAPGSPAGSPTSTEEGTSTE PSEGSAPGTSTEPSEGSAPGTSESATPESGPGSEPATSGGSGGS (SEQ ID NO: 151). In some embodiments, the linker is 24 amino acids in length. In some embodiments, the linker comprises the amino acid sequence SGGSSGGSSGSETPGTSESATPES (SEQ ID NO: 146). In some embodiments, the linker is 40 amino acids in length. In some embodiments, the linker comprises the amino acid sequence SGGSSGGSSGSETPGTSESATPESSGGSSGGSSGGSSGGS (SEQ ID NO: 148). In some embodiments, the linker is 64 amino acids in length. In some embodiments, the linker comprises the amino acid sequence SGGSSGGSSGSETPGTSESATPESSGGSSGGSSGGSSGGSSGSETPGTSESATPESSGGS SGGS (SEQ ID NO: 149). In some embodiments, the linker is 92 amino acids in length. In some embodiments, the linker comprises the amino acid sequence PGSPAGSPTSTEEGTSESATPESGPGTSTEPSEGSAPGSPAGSPTSTEEGTSTEPSEGSAP GTSTEPSEGSAPGTSESATPESGPGSEPATS (SEQ ID NO: 150). It should be appreciated that any of the linkers provided herein may be used to link a first adenosine deaminase and a second adenosine deaminase; an adenosine deaminase (e.g., a first or a second adenosine deaminase) and a napDNAbp; a napDNAbp and an NLS; or an adenosine deaminase (e.g., a first or a second adenosine deaminase) and an NLS.

In some embodiments, any of the fusion proteins provided herein, comprise an adenosine or a cytidine deaminase and a napDNAbp that are fused to each other via a linker. In some embodiments, any of the fusion proteins provided herein, comprise a first adenosine deaminase and a second adenosine deaminase that are fused to each other via a linker. In some embodiments, any of the fusion proteins provided herein, comprise an NLS, which may be fused to an adenosine deaminase (e.g., a first and/or a second adenosine deaminase), a nucleic acid programmable DNA binding protein (napDNAbp). Various linker lengths and flexibilities between an adenosine deaminase (e.g., an engineered ecTadA) and a napDNAbp (e.g., a Cas9 domain), and/or between a first adenosine deaminase and a second adenosine deaminase can be employed (e.g., ranging from very flexible linkers of the form (GGGGS)ₙ (SEQ ID NO: 141), (GGGGS)ₙ (SEQ ID NO: 141), and (G)ₙ (SEQ ID NO: 135) to more rigid linkers of the form (EAAAK)ₙ (SEQ ID NO: 142), (SGGS)ₙ (SEQ ID NO: 139), SGSETPGTSESATPES (SEQ ID NO: 143) (see, e.g., Guilinger J P, Thompson D B, Liu D R. Fusion of catalytically inactive Cas9 to FokI nuclease improves the specificity of genome modification. Nat. Biotechnol. 2014; 32(6): 577-82; the entire contents are incorporated herein by reference) and (XP)ₙ (SEQ ID NO: 136)) in order to achieve the optimal length for deaminase activity for the specific application. In some embodiments, n is 1, 2, 3, 4, 5, 6, 7, 8, 9, 10, 11, 12, 13, 14, or 15. In some embodiments, the linker comprises a (GGS)ₙ (SEQ ID NO: 137) motif, wherein n is 1, 3, or 7. In some embodiments, the adenosine deaminase and the napDNAbp, and/or the first adenosine deaminase and the second adenosine deaminase of any of the fusion proteins provided herein are fused via a linker comprising the amino acid sequence SGSETPGTSESATPES (SEQ ID NO: 143), SGGS (SEQ ID NO: 138), SGGSSGSETPGTSESATPESSGGS (SEQ ID NO: 145), SGGSSGGSSGSETPGTSESATPESSGGSSGGS (SEQ ID NO: 144), or GGSGGSPGSPAGSPTSTEEGTSESATPESGPGTSTEPSEGSAPGSPAGSPTSTEEGTSTE PSEGSAPGTSTEPSEGSAPGTSESATPESGPGSEPATSGGSGGS (SEQ ID NO: 151). In some embodiments, the linker is 24 amino acids in length. In some embodiments, the linker comprises the amino acid sequence SGGSSGGSSGSETPGTSESATPES (SEQ ID NO: 146). In some embodiments, the linker is 32 amino acids in length. In some embodiments, the linker is 32 amino acids in length. In some embodiments, the linker comprises the amino acid sequence (SGGS)₂—SGSETPGTSESATPES-(SGGS)₂ (SEQ ID NO: 144), which may also be referred to as (SGGS)₂—XTEN-(SGGS)₂ (SEQ ID NO: 144). In some embodiments, the linker comprises the amino acid sequence, wherein n is 0, 1, 2, 3, 4, 5, 6, 7, 8, 9, or 10. In some embodiments, the linker is 40 amino acids in length. In some embodiments, the linker comprises the amino acid sequence SGGSSGGSSGSETPGTSESATPESSGGSSGGSSGGSSGGS (SEQ ID NO: 148). In some embodiments, the linker is 64 amino acids in length. In some embodiments, the linker comprises the amino acid sequence SGGSSGGSSGSETPGTSESATPESSGGSSGGSSGGSSGGSSGSETPGTSESATPESSGGS SGGS (SEQ ID NO: 149). In some embodiments, the linker is 92 amino acids in length. In some embodiments, the linker comprises the amino acid sequence

(SEQ ID NO: 150)
PGSPAGSPTSTEEGTSESATPESGPGTSTEPSEGS
APGSPAGSPTSTEEGTSTEPSEGSAPGTSTEPSEG
SAPGTSESATPESGPGSEPATS.

(2) UGI Domain

In other embodiments, the base editors described herein may comprise one or more uracil glycosylase inhibitors. The term “uracil glycosylase inhibitor” or “UGI,” as used herein, refers to a protein that is capable of inhibiting a uracil-DNA glycosylase base-excision repair enzyme. In some embodiments, a UGI domain comprises a wild-type UGI or a UGI as set forth in SEQ ID NO: 163. In some embodiments, the UGI proteins provided herein include fragments of UGI and proteins homologous to a UGI or a UGI fragment. For example, in some embodiments, a UGI domain comprises a fragment of the amino acid sequence set forth in SEQ ID NO: 163. In some embodiments, a UGI fragment comprises an amino acid sequence that comprises at least 60%, at least 65%, at least 70%, at least 75%, at least 80%, at least 85%, at least 90%, at least 95%, at least 96%, at least 97%, at least 98%, at least 99%, or at least 99.5% of the amino acid sequence as set forth in SEQ ID NO: 163. In some embodiments, a UGI comprises an amino acid sequence homologous to the amino acid sequence set forth in SEQ ID NO: 163, or an amino acid sequence homologous to a fragment of the amino acid sequence set forth in SEQ ID NO: 163. In some embodiments, proteins comprising UGI or fragments of UGI or homologs of UGI or UGI fragments are referred to as “UGI variants.” A UGI variant shares homology to UGI, or a fragment thereof. For example a UGI variant is at least 70% identical, at least 75% identical, at least 80% identical, at least 85% identical, at least 90% identical, at least 95% identical, at least 96% identical, at least 97% identical, at least 98% identical, at least 99% identical, at least 99.5% identical, or at least 99.9% identical to a wild type UGI or a UGI as set forth in SEQ ID NO: 163. In some embodiments, the UGI variant comprises a fragment of UGI, such that the fragment is at least 70% identical, at least 80% identical, at least 90% identical, at least 95% identical, at least 96% identical, at least 97% identical, at least 98% identical, at least 99% identical, at least 99.5% identical, or at least 99.9% to the corresponding fragment of wild-type UGI or a UGI as set forth in SEQ ID NO: 163. In some embodiments, the UGI comprises the following amino acid sequence:

Uracil-DNA glycosylase inhibitor:
>spP14739UNGI_BPPB2
(SEQ ID NO: 163)
MTNLSDIIEKETGKQLVIQESILMLPEEVEEVIGN
KPESDILVHTAYDESTDENVMLLTSDAPEYKPWAL
VIQDSNGENKIKML.

The base editors described herein may comprise more than one UGI domain, which may be separated by one or more linkers as described herein. It will also be understood that in the context of the herein disclosed base editors, the UGI domain may be linked to a deaminase domain or

### (3) NLS Domains

In various embodiments, the PE fusion proteins may comprise one or more nuclear localization sequences (NLS), which help promote translocation of a protein into the cell nucleus. Such sequences are well-known in the art and can include the following examples:

| | | SEQ |
|---|---|---|
| | | ID |
| DESCRIPTION | SEQUENCE | NO: |
| NLS OF SV40 | PKKKRKV | 152 |
| LARGE T-AG | | |
| NLS OF | VSRKRPRP | 153 |
| POLYOMA | | |
| LARGE T-AG | | |
| NLS OF C- | PAAKRVKLD | 154 |
| MYC | | |
| NLS OF TUS- | KLKIKRPVK | 155 |
| PROTEIN | | |
| NLS OF | EGAPPAKRAR | 156 |
| HEPATITIS D | | |
| VIRUS | | |
| ANTIGEN | | |
| NLS OF | PPQPKKKPLDGE | 157 |
| MURINE P53 | | |
| NLS | MKRTADGSEFESPKKKRKV | 158 |
| NLS OF | AVKRPAATKKAGQAKKKKLD | 159 |
| NUCLEOPLASM | | |
| IN | | |
| NLS OF PEI | SGGSKRTADGSEFEPKKKRKV | 160 |
| AND PE2 | | |
| NLS OF EGL- | MSRRRKANPTKLSENAKKLAK | 161 |
| 13 | EVEN | |
| NLS | MDSLLMNRRKFLYQFKNVRW | 162 |
| | AKGRRETYLC | |

The NLS examples above are non-limiting. The PE fusion proteins may comprise any known NLS sequence, including any of those described in Cokol et al., “Finding nuclear localization signals,” EMBO Rep., 2000, 1(5): 411-415 and Freitas et al., “Mechanisms and Signals for the Nuclear Import of Proteins,” Current Genomics, 2009, 10(8): 550-7, each of which are incorporated herein by reference.

### (4) Split-Intein Domains

It will be understood that in some embodiments (e.g., delivery of a base editor in vivo using AAV particles), it may be advantageous to split a polypeptide (e.g., a deaminase or a napDNAbp) or a fusion protein (e.g., a base editor) into an N-terminal half and a C-terminal half, delivery them separately, and then allow their colocalization to reform the complete protein (or fusion protein as the case may be) within the cell. Separate halves of a protein or a fusion protein may each comprise a split-intein tag to facilitate the reformation of the complete protein or fusion protein by the mechanism of protein trans splicing.

Protein trans-splicing, catalyzed by split inteins, provides an entirely enzymatic method for protein ligation. A split-intein is essentially a contiguous intein (e.g. a mini-intein) split into two pieces named N-intein and C-intein, respectively. The N-intein and C-intein of a split intein can associate non-covalently to form an active intein and catalyze the splicing reaction essentially in same way as a contiguous intein does. Split inteins have been found in nature and also engineered in laboratories. As used herein, the term “split intein” refers to any intein in which one or more peptide bond breaks exists between the N-terminal and C-terminal amino acid sequences such that the N-terminal and C-terminal sequences become separate molecules that can non-covalently reassociate, or reconstitute, into an intein that is functional for trans-splicing reactions. Any catalytically active intein, or fragment thereof, may be used to derive a split intein for use in the methods of the invention. For example, in one aspect the split intein may be derived from a eukaryotic intein. In another aspect, the split intein may be derived from a bacterial intein. In another aspect, the split intein may be derived from an archaeal intein. Preferably, the split intein so-derived will possess only the amino acid sequences essential for catalyzing trans-splicing reactions.

As used herein, the “N-terminal split intein (In)” refers to any intein sequence that comprises an N-terminal amino acid sequence that is functional for trans-splicing reactions. An In thus also comprises a sequence that is spliced out when trans-splicing occurs. An In can comprise a sequence that is a modification of the N-terminal portion of a naturally occurring intein sequence. For example, an In can comprise additional amino acid residues and/or mutated residues so long as the inclusion of such additional and/or mutated residues does not render the In non-functional in trans-splicing. Preferably, the inclusion of the additional and/or mutated residues improves or enhances the trans-splicing activity of the In.

As used herein, the “C-terminal split intein (Ic)” refers to any intein sequence that comprises a C-terminal amino acid sequence that is functional for trans-splicing reactions. In one aspect, the Ic comprises 4 to 7 contiguous amino acid residues, at least 4 amino acids of which are from the last β-strand of the intein from which it was derived. An Ic thus also comprises a sequence that is spliced out when trans-splicing occurs. An Ic can comprise a sequence that is a modification of the C-terminal portion of a naturally occurring intein sequence. For example, an Ic can comprise additional amino acid residues and/or mutated residues so long as the inclusion of such additional and/or mutated residues does not render the In non-functional in trans-splicing. Preferably, the inclusion of the additional and/or mutated residues improves or enhances the trans-splicing activity of the Ic.

In some embodiments of the invention, a peptide linked to an Ic or an In can comprise an additional chemical moiety including, among others, fluorescence groups, biotin, polyethylene glycol (PEG), amino acid analogs, unnatural amino acids, phosphate groups, glycosyl groups, radioisotope labels, and pharmaceutical molecules. In other embodiments, a peptide linked to an Ic can comprise one or more chemically reactive groups including, among others, ketone, aldehyde, Cys residues and Lys residues. The N-intein and C-intein of a split intein can associate non-covalently to form an active intein and catalyze the splicing reaction when an “intein-splicing polypeptide (ISP)” is present. As used herein, “intein-splicing polypeptide (ISP)” refers to the portion of the amino acid sequence of a split intein that remains when the Ic, In, or both, are removed from the split intein. In certain embodiments, the In comprises the ISP. In another embodiment, the Ic comprises the ISP. In yet another embodiment, the ISP is a separate peptide that is not covalently linked to In nor to Ic.

Split inteins may be created from contiguous inteins by engineering one or more split sites in the unstructured loop or intervening amino acid sequence between the −12 conserved beta-strands found in the structure of mini-inteins. Some flexibility in the position of the split site within regions between the beta-strands may exist, provided that creation of the split will not disrupt the structure of the intein, the structured beta-strands in particular, to a sufficient degree that protein splicing activity is lost.

In protein trans-splicing, one precursor protein consists of an N-extein part followed by the N-intein, another precursor protein consists of the C-intein followed by a C-extein part, and a trans-splicing reaction (catalyzed by the N- and C-inteins together) excises the two intein sequences and links the two extein sequences with a peptide bond. Protein trans-splicing, being an enzymatic reaction, can work with very low (e.g. micromolar) concentrations of proteins and can be carried out under physiological conditions.

### (5) RNA-Protein Recruitment System

In various embodiments, two separate protein domains (e.g., a Cas9 domain and a cytidine deaminase domain) may be colocalized to one another to form a functional complex (akin to the function of a fusion protein comprising the two separate protein domains) by using an “RNA-protein recruitment system,” such as the “MS2 tagging technique.” Such systems generally tag one protein domain with an “RNA-protein interaction domain” (aka “RNA-protein recruitment domain”) and the other with an “RNA-binding protein” that specifically recognizes and binds to the RNA-protein interaction domain, e.g., a specific hairpin structure. These types of systems can be leveraged to colocalize the domains of a base editor, as well as to recruitment additional functionalities to a base editor, such as a UGI domain. In one example, the MS2 tagging technique is based on the natural interaction of the MS2 bacteriophage coat protein (“MCP” or “MS2cp”) with a stem-loop or hairpin structure present in the genome of the phage, i.e., the “MS2 hairpin.” In the case of the MS2 hairpin, it is recognized and bound by the MS2 bacteriophage coat protein (MCP). Thus, in one exemplary scenario a deaminase-MS2 fusion can recruit a Cas9-MCP fusion.

A review of other modular RNA-protein interaction domains are described in the art, for example, in Johansson et al., “RNA recognition by the MS2 phage coat protein,” Sem Virol., 1997, Vol. 8(3): 176-185; Delebecque et al., “Organization of intracellular reactions with rationally designed RNA assemblies,” Science, 2011, Vol. 333: 470-474; Mali et al., “Cas9 transcriptional activators for target specificity screening and paired nickases for cooperative genome engineering,” Nat. Biotechnol., 2013, Vol. 31: 833-838; and Zalatan et al., “Engineering complex synthetic transcriptional programs with CRISPR RNA scaffolds,” Cell, 2015, Vol. 160: 339-350, each of which are incorporated herein by reference in their entireties. Other systems include the PP7 hairpin, which specifically recruits the PCP protein, and the “com” hairpin, which specifically recruits the Com protein. See Zalatan et al.

The nucleotide sequence of the MS2 hairpin (or equivalently referred to as the “MS2 aptamer”) is: GCCAACATGAGGATCACCCATGTCTGCAGGGCC (SEQ ID NO: 172).

The amino acid sequence of the MCP or MS2cp is:

(SEQ ID NO: 173)
GSASNFTQFVLVDNGGTGDVTVAPSNFANGVAEWISSNSR
SQAYKVTCSVRQSSAQNRKYTIKVEVPKVATQTVGGEELP
VAGWRSYLNMELTIPIFATNSDCELIVKAMQGLLKDGNPI
PSAIAANSGIY.

VI. Base Editors

In various aspects, the instant specification provides base editors and methods of using the same, along with a suitable guide RNA, to edit target DNA in a manner predicted by the herein disclosed computational modes by installing precise nucleobase changes in target sequences.

The state of the art has described numerous base editors as of this filing. It will be understood that the methods and approaches herein described for editing the gene loci may be applied to any previously known base editor, or to base editors that may be developed or evolved in the future.

Exemplary base editors that may be used in accordance with the present disclosure include those described in the following references and/or patent publications, each of which are incorporated by reference in their entireties: (a) PCT/US2014/070038 (published as WO2015/089406, Jun. 18, 2015) and its equivalents in the US or around the world; (b) PCT/US2016/058344 (published as WO2017/070632, Apr. 27, 2017) and its equivalents in the US or around the world; (c) PCT/US2016/058345 (published as WO2017/070633, April 27. 2017) and its equivalent in the US or around the world; (d) PCT/US2017/045381 (published as WO2018/027078, Feb. 8, 2018) and its equivalents in the US or around the world; (e) PCT/US2017/056671 (published as WO2018/071868, Apr. 19, 2018) and its equivalents in the US or around the world; PCT/2017/048390 (WO2017/048390, Mar. 23, 2017) and its equivalents in the US or around the world; (f) PCT/US2017/068114 (not published) and its equivalents in the US or around the world; (g) PCT/US2017/068105 (not published) and its equivalents in the US or around the world; (h) PCT/US2017/046144 (WO2018/031683, Feb. 15, 2018) and its equivalents in the US or around the world; (i) PCT/US2018/024208 (not published) and its equivalents in the US or around the world; (j) PCT/2018/021878 (WO2018/021878, Feb. 1, 2018) and its equivalents in the US and around the world; (k) Komor, A. C., Kim, Y. B., Packer, M. S., Zuris, J. A. & Liu, D. R. Programmable editing of a target base in genomic DNA without double-stranded DNA cleavage. Nature 533, 420-(2016); (1) Gaudelli, N. M. et al. Programmable base editing of A.T to G.C in genomic DNA without DNA cleavage. Nature 551, 464- (2017); (m) any of the references listed in this specification entitled “References” and which reports or describes a base editor known in the art.

In various aspects, the improved or modified base editors described herein have the following generalized structures:
[A]-[B] or [B]-[A],

wherein [A] is a napDNAbp and [B] is nucleic acid effector domain (e.g., an adenosine deaminase, or cytidine deaminase), and “]-[” represents an optional a linker that joins the [A] and [B] domains together, either covalently or non-covalently.

Such base editors may also comprising one or more additional functional moieties, [C], such as UGI domains or NLS domains, joined optionally through a linker to [A] and/or [B].

In some embodiments, the base editors provided herein can be made as a recombinant fusion protein comprising one or more protein domains, thereby generating a base editor. In certain embodiments, the base editors provided herein comprise one or more features that improve the base editing activity (e.g., efficiency, selectivity, and/or specificity) of the base editor proteins. For example, the base editor proteins provided herein may comprise a Cas9 domain that has reduced nuclease activity. In some embodiments, the base editor proteins provided herein may have a Cas9 domain that does not have nuclease activity (dCas9), or a Cas9 domain that cuts one strand of a duplexed DNA molecule, referred to as a Cas9 nickase (nCas9). Without wishing to be bound by any particular theory, the presence of the catalytic residue (e.g., H840) maintains the activity of the Cas9 to cleave the non-edited (e.g., non-deaminated) strand containing a T opposite the targeted A. Mutation of the catalytic residue (e.g., D10 to A10) of Cas9 prevents cleavage of the edited strand containing the targeted A residue. Such Cas9 variants are able to generate a single-strand DNA break (nick) at a specific location based on the gRNA-defined target sequence, leading to repair of the non-edited strand, ultimately resulting in a T to C change on the non-edited strand.

In particular, the disclosure provides adenosine base editors that can be used to correct a mutation or install a genetic change. Exemplary domains used in base editing fusion proteins, including adenosine deaminases, napDNA/RNAbp (e.g., Cas9), and nuclear localization sequences (NLSs) are described in further detail below.

Some aspects of the disclosure provide fusion proteins comprising a nucleic acid programmable DNA binding protein (napDNAbp) and an adenosine deaminase. In some embodiments, any of the fusion proteins provided herein is a base editor. In some embodiments, the napDNAbp is a Cas9 domain, a Cpf1 domain, a CasX domain, a CasY domain, a C2c1 domain, a C2c2 domain, aC2c3 domain, or an Argonaute domain. In some embodiments, the napDNAbp is any napDNAbp provided herein. Some aspects of the disclosure provide fusion proteins comprising a Cas9 domain and an adenosine deaminase. The Cas9 domain may be any of the Cas9 domains or Cas9 proteins (e.g., dCas9 or nCas9) provided herein. In some embodiments, any of the Cas9 domains or Cas9 proteins (e.g., dCas9 or nCas9) provided herein may be fused with any of the deaminases provided herein. In some embodiments, the fusion protein comprises the structure:
NH₂-[deaminase]-[napDNAbp]-COOH; or
NH₂-[napDNAbp]-[deaminase]-COOH

In some embodiments, the fusion proteins comprising an deaminase and a napDNAbp (e.g., Cas9 domain) do not include a linker sequence. In some embodiments, a linker is present between the deaminase domain and the napDNAbp. In some embodiments, the “]-[” used in the general architecture above indicates the presence of an optional linker. In some embodiments, the deaminase and the napDNAbp are fused via any of the linkers provided herein. For example, in some embodiments the deaminase and the napDNAbp are fused via any of the linkers provided below in the section entitled “Linkers”. In some embodiments, the deaminase and the napDNAbp are fused via a linker that comprises between 1 and 200 amino acids. In some embodiments, the adenosine deaminase and the napDNAbp are fused via a linker that comprises from 1 to 5, 1 to 10, 1 to 20, 1 to 30, 1 to 40, 1 to 50, 1 to 60, 1 to 80, 1 to 100, 1 to 150, 1 to 200, 5 to 10, 5 to 20, 5 to 30, 5 to 40, 5 to 60, 5 to 80, 5 to 100, 5 to 150, 5 to 200, 10 to 20, 10 to 30, 10 to 40, 10 to 50, 10 to 60, 10 to 80, 10 to 100, 10 to 150, 10 to 200, 20 to 30, 20 to 40, 20 to 50, 20 to 60, 20 to 80, 20 to 100, 20 to 150, 20 to 200, 30 to 40, 30 to 50, 30 to 60, 30 to 80, 30 to 100, 30 to 150, 30 to 200, 40 to 50, 40 to 60, 40 to 80, 40 to 100, 40 to 150, 40 to 200, 50 to 60 50 to 80, 50 to 100, 50 to 150, 50 to 200, 60 to 80, 60 to 100, 60 to 150, 60 to 200, 80 to 100, 80 to 150, 80 to 200, 100 to 150, 100 to 200, or 150 to 200 amino acids in length. In some embodiments, the adenosine deaminase and the napDNAbp are fused via a linker that comprises 3, 4, 16, 24, 32, 64, 100, or 104 amino acids in length.

In some embodiments, the based editors provided herein further comprise one or more nuclear targeting sequences, for example, a nuclear localization sequence (NLS). In some embodiments, a NLS comprises an amino acid sequence that facilitates the importation of a protein, that comprises an NLS, into the cell nucleus (e.g., by nuclear transport). In some embodiments, any of the fusion proteins provided herein further comprise a nuclear localization sequence (NLS). In some embodiments, the NLS is fused to the N-terminus of the fusion protein. In some embodiments, the NLS is fused to the C-terminus of the fusion protein. In some embodiments, the NLS is fused to the N-terminus of the napDNAbp. In some embodiments, the NLS is fused to the C-terminus of the napDNAbp. In some embodiments, the NLS is fused to the N-terminus of the adenosine deaminase. In some embodiments, the NLS is fused to the C-terminus of the adenosine deaminase. In some embodiments, the NLS is fused to the fusion protein via one or more linkers. In some embodiments, the NLS is fused to the fusion protein without a linker. In some embodiments, the NLS comprises an amino acid sequence of any one of the NLS sequences provided or referenced herein. In some embodiments, the NLS comprises an amino acid sequence as set forth in any one of SEQ ID NOs: 152-162. Additional nuclear localization sequences are known in the art and would be apparent to the skilled artisan. For example, NLS sequences are described in Plank et al., PCT/EP2000/011690, the contents of which are incorporated herein by reference for their disclosure of exemplary nuclear localization sequences.

In some embodiments, the general architecture of exemplary fusion proteins with an deaminase and a napDNAbp comprises any one of the following structures, where NLS is a nuclear localization sequence (e.g., any NLS provided herein), NH₂ is the N-terminus of the fusion protein, and COOH is the C-terminus of the fusion protein. Fusion proteins comprising an adenosine deaminase, a napDNAbp, and a NLS:
NH₂-[NLS]-[deaminase]-[napDNAbp]-COOH;
NH₂-[deaminase]-[NLS]-[napDNAbp]-COOH;
NH₂-[deaminase]-[napDNAbp]-[NLS]-COOH;
NH₂-[NLS]-[napDNAbp]-[deaminase]-COOH;
NH₂-[napDNAbp]-[NLS]-[deaminase]-COOH; and
NH₂-[napDNAbp]-[deaminase]-[NLS]-COOH.

Some aspects of the disclosure provide ABEs (adenine base editors) that comprise a nucleic acid programmable DNA binding protein (napDNAbp) and at least two adenosine deaminase domains. Without wishing to be bound by any particular theory, dimerization of adenosine deaminases (e.g., in cis or in trans) may improve the ability (e.g., efficiency) of the fusion protein to modify a nucleic acid base, for example to deaminate adenine. In some embodiments, any of the fusion proteins may comprise 2, 3, 4 or 5 adenosine deaminase domains. In some embodiments, any of the fusion proteins provided herein comprise two adenosine deaminases. In some embodiments, any of the fusion proteins provided herein contain only two adenosine deaminases. In some embodiments, the adenosine deaminases are the same. In some embodiments, the adenosine deaminases are any of the adenosine deaminases provided herein. In some embodiments, the adenosine deaminases are different. In some embodiments, the first adenosine deaminase is any of the adenosine deaminases provided herein, and the second adenosine is any of the adenosine deaminases provided herein, but is not identical to the first adenosine deaminase. As one example, the fusion protein may comprise a first adenosine deaminase and a second adenosine deaminase that both comprise the amino acid sequence of SEQ ID NO: 91, which contains a W23R; H36L; P48A; R51L; L84F; A106V; D108N; H123Y; S146C; D147Y; R152P; E155V; I156F; and K157N mutation from ecTadA (SEQ ID NO: 89). In some embodiments, the fusion protein may comprise a first adenosine deaminase that comprises the amino acid sequence, e.g., of SEQ ID NO: 89, and a second adenosine deaminase domain that comprises the amino amino acid sequence of TadA7.10 of SEQ ID NO: 79. Additional fusion protein constructs comprising two adenosine deaminase domains are illustrated herein and are provided in the art.

In some embodiments, the fusion protein comprises two adenosine deaminases (e.g., a first adenosine deaminase and a second adenosine deaminase). In some embodiments, the fusion protein comprises a first adenosine deaminase and a second adenosine deaminase. In some embodiments, the first adenosine deaminase is N-terminal to the second adenosine deaminase in the fusion protein. In some embodiments, the first adenosine deaminase is C-terminal to the second adenosine deaminase in the fusion protein. In some embodiments, the first adenosine deaminase and the second deaminase are fused directly or via a linker. In some embodiments, the linker is any of the linkers provided herein, for example, any of the linkers described in the “Linkers” section.

In some embodiments, the first adenosine deaminase is the same as the second adenosine deaminase. In some embodiments, the first adenosine deaminase and the second adenosine deaminase are any of the adenosine deaminases described herein. In some embodiments, the first adenosine deaminase and the second adenosine deaminase are different. In some embodiments, the first adenosine deaminase is any of the adenosine deaminases provided herein. In some embodiments, the second adenosine deaminase is any of the adenosine deaminases provided herein but is not identical to the first adenosine deaminase. In some embodiments, the first adenosine deaminase is an ecTadA adenosine deaminase. In some embodiments, the first adenosine deaminase comprises an amino acid sequence that is at least 60%, at least 65%, at least 70%, at least 75%, at least 80%, at least 85%, at least 90%, at least 95%, at least 96%, at least 97%, at least 98%, at least 99%, or at least 99.5% identical to any one of the amino acid sequences set forth in any one of SEQ ID NOs: 78-91, and 403-404, or to any of the adenosine deaminases provided herein. In some embodiments, the first adenosine deaminase comprises an amino acid sequence, e.g., of SEQ ID NO: 78-91, and 403-404. In some embodiments, the second adenosine deaminase comprises an amino acid sequence that is at least 60%, at least 65%, at least 70%, at least 75%, at least 80%, at least 85%, at least 90%, at least 95%, at least 96%, at least 97%, at least 98%, at least 99%, or at least 99.5% identical to any one of the amino acid sequences set forth in any one of SEQ ID NOs: 78-91, and 403-404, or to any of the deaminases provided herein. The amino acid sequences can be the same or different. In some embodiments, the second adenosine deaminase comprises an amino acid sequence of any one of SEQ ID NOs: 78-91, and 403-404.

In some embodiments, the general architecture of exemplary fusion proteins with a first adenosine deaminase, a second adenosine deaminase, and a napDNAbp comprises any one of the following structures, where NLS is a nuclear localization sequence (e.g., any NLS provided herein), NH₂ is the N-terminus of the fusion protein, and COOH is the C-terminus of the fusion protein.

Thus, in some embodiments, the disclosure provides based editors comprising a first adenosine deaminase, a second adenosine deaminase, and a napDNAbp, such as: NH₂-[first adenosine deaminase]-[second adenosine deaminase]-[napDNAbp]-COOH; NH₂-[first adenosine deaminase]-[napDNAbp]-[second adenosine deaminase]-COOH; NH₂-[napDNAbp]-[first adenosine deaminase]-[second adenosine deaminase]-COOH; NH₂-[second adenosine deaminase]-[first adenosine deaminase]-[napDNAbp]-COOH; NH₂-[second adenosine deaminase]-[napDNAbp]-[first adenosine deaminase]-COOH; NH₂-[napDNAbp]-[second adenosine deaminase]-[first adenosine deaminase]-COOH;

In some embodiments, the fusion proteins provided herein do not comprise a linker. In some embodiments, a linker is present between one or more of the domains or proteins (e.g., first adenosine deaminase, second adenosine deaminase, and/or napDNAbp). In some embodiments, the “-” used in the general architecture above indicates the presence of an optional linker.

In other embodiments, the disclosure provides based editors comprising a first adenosine deaminase, a second adenosine deaminase, a napDNAbp, and an NLS, such as: NH₂-[NLS]-[first adenosine deaminase]-[second adenosine deaminase]-[napDNAbp]-COOH; NH₂-[first adenosine deaminase]-[NLS]-[second adenosine deaminase]-[napDNAbp]-COOH; NH₂-[first adenosine deaminase]-[second adenosine deaminase]-[NLS]-[napDNAbp]-COOH; NH₂-[first adenosine deaminase]-[second adenosine deaminase]-[napDNAbp]-[NLS]-COOH; NH₂-[NLS]-[first adenosine deaminase]-[napDNAbp]-[second adenosine deaminase]-COOH; NH₂-[first adenosine deaminase]-[NLS]-[napDNAbp]-[second adenosine deaminase]-COOH; NH₂-[first adenosine deaminase]-[napDNAbp]-[NLS]-[second adenosine deaminase]-COOH; NH₂-[first adenosine deaminase]-[napDNAbp]-[second adenosine deaminase]-[NLS]-COOH; NH₂-[NLS]-[napDNAbp]-[first adenosine deaminase]-[second adenosine deaminase]-COOH; NH₂-[napDNAbp]-[NLS]-[first adenosine deaminase]-[second adenosine deaminase]-COOH; NH₂-[napDNAbp]-[first adenosine deaminase]-[NLS]-[second adenosine deaminase]-COOH; NH₂-[napDNAbp]-[first adenosine deaminase]-[second adenosine deaminase]-[NLS]-COOH; NH₂-[NLS]-[second adenosine deaminase]-[first adenosine deaminase]-[napDNAbp]-COOH; NH₂-[second adenosine deaminase]-[NLS]-[first adenosine deaminase]-[napDNAbp]-COOH; NH₂-[second adenosine deaminase]-[first adenosine deaminase]-[NLS]-[napDNAbp]-COOH; NH₂-[second adenosine deaminase]-[first adenosine deaminase]-[napDNAbp]-[NLS]-COOH; NH₂-[NLS]-[second adenosine deaminase]-[napDNAbp]-[first adenosine deaminase]-COOH; NH₂-[second adenosine deaminase]-[NLS]-[napDNAbp]-[first adenosine deaminase]-COOH; NH₂-[second adenosine deaminase]-[napDNAbp]-[NLS]-[first adenosine deaminase]-COOH; NH₂-[second adenosine deaminase]-[napDNAbp]-[first adenosine deaminase]-[NLS]-COOH; NH₂-[NLS]-[napDNAbp]-[second adenosine deaminase]-[first adenosine deaminase]-COOH; NH₂-[napDNAbp]-[NLS]-[second adenosine deaminase]-[first adenosine deaminase]-COOH; NH₂-[napDNAbp]-[second adenosine deaminase]-[NLS]-[first adenosine deaminase]-COOH; NH₂-[napDNAbp]-[second adenosine deaminase]-[first adenosine deaminase]-[NLS]-COOH;

In some embodiments, the fusion proteins provided herein do not comprise a linker. In some embodiments, a linker is present between one or more of the domains or proteins (e.g., first adenosine deaminase, second adenosine deaminase, napDNAbp, and/or NLS). In some embodiments, the “-” used in the general architecture above indicates the presence of an optional linker.

It should be appreciated that the fusion proteins of the present disclosure may comprise one or more additional features. For example, in some embodiments, the fusion protein may comprise cytoplasmic localization sequences, export sequences, such as nuclear export sequences, or other localization sequences, as well as sequence tags that are useful for solubilization, purification, or detection of the fusion proteins. Suitable protein tags provided herein include, but are not limited to, biotin carboxylase carrier protein (BCCP) tags, myc-tags, calmodulin-tags, FLAG-tags, hemagglutinin (HA)-tags, polyhistidine tags, also referred to as histidine tags or His-tags, maltose binding protein (MBP)-tags, nus-tags, glutathione-S-transferase (GST)-tags, green fluorescent protein (GFP)-tags, thioredoxin-tags, S-tags, Softags (e.g., Softag 1, Softag 3), strep-tags, biotin ligase tags, FlAsH tags, V5 tags, and SBP-tags. Additional suitable sequences will be apparent to those of skill in the art. In some embodiments, the fusion protein comprises one or more His tags.

Base Editors Used in Training BE-Hive in Connection with Example 1

The following CBEs were used to generate training data for the BE-Hive algorithm of Example 1. Each of the CBEs have the same architecture of [NLS]-[deaminase]-[Cas9]-[UGI]-[UGI]-[NLS] (which is the BE4max architecture) and with interchangeable deaminases.

In addition, Cas-protein components of these editors can include SpCas9, SpCas9 circular permutant 1028, or Cas9-NG. Amino acid sequences are provided for the BE4 (BE4max) construct as an example, and separately amino acid sequences for deaminases and Cas9 proteins are provided below.

| | | SEQ ID |
|---|---|---|
| DESCRIPTION | SEQUENCE | NO: |
| BE4max (or BE4) | MKRTADGSEFESPKKKRKVSSETGPVAVDPTLRRRIEPHEFEVFFDPRELRKETCLLYE | 3200 |
| Cas9 = SpCas9 | INWGGRHSIWRHTSQNTNKHVEVNFIEKFTTERYFCPNTRCSITWFLSWSPCGECSRAI | |
| | TEFLSRYPHVTLFIYIARLYHHADPRNRQGLRDLISSGVTIQIMTEQESGYCWRNFVNY | |
| | SPSNEAHWPRYPHLWVRLYVLELYCIILGLPPCLNILRRKQPQLTFFTIALQSCHYQRL | |
| | PPHILWATGLKSGGSSGGSSGSETPGTSESATPESSGGSSGGDKKYSIGLAIGTNSVGW | |
| | AVITDEYKVPSKKFKVLGNTDRHSIKKNLIGALLFDSGETAEATRLKRTARRRYTRRKN | |
| | RICYLQEIFSNEMAKVDDSFFHRLEESFLVEEDKKHERHPIFGNIVDEVAYHEKYPTIY | |
| | HLRKKLVDSTDKADLRLIYLALAHMIKFRGHFLIEGDLNPDNSDVDKLFIQLVQTYNQL | |
| | FEENPINASGVDAKAILSARLSKSRRLENLIAQLPGEKKNGLFGNLIALSLGLTPNFKS | |
| | NFDLAEDAKLQLSKDTYDDDLDNLLAQIGDQYADLFLAAKNLSDAILLSDILRVNTEIT | |
| | KAPLSASMIKRYDEHHQDLTLLKALVRQQLPEKYKEIFFDQSKNGYAGYIDGGASQEEF | |
| | YKFIKPILEKMDGTEELLVKLNREDLLRKQRTFDNGSIPHQIHLGELHAILRRQEDFYP | |
| | FLKDNREKIEKILTFRIPYYVGPLARGNSRFAWMTRKSEETITPWNFEEVVDKGASAQS | |
| | FIERMTNFDKNLPNEKVLPKHSLLYEYFTVYNELTKVKYVTEGMRKPAFLSGEQKKAIV | |
| | DLLFKTNRKVTVKQLKEDYFKKIECFDSVEISGVEDRFNASLGTYHDLLKIIKDKDFLD | |
| | NEENEDILEDIVLTLTLFEDREMIEERLKTYAHLFDDKVMKQLKRRRYTGWGRLSRKLI | |
| | NGIRDKQSGKTILDFLKSDGFANRNFMQLIHDDSLTFKEDIQKAQVSGQGDSLHEHIAN | |
| | LAGSPAIKKGILQTVKVVDELVKVMGRHKPENIVIEMARENQTTQKGQKNSRERMKRIE | |
| | EGIKELGSQILKEHPVENTQLQNEKLYLYYLQNGRDMYVDQELDINRLSDYDVDHIVPQ | |
| | SFLKDDSIDNKVLTRSDKNRGKSDNVPSEEVVKKMKNYWRQLLNAKLITQRKFDNLTKA | |
| | ERGGLSELDKAGFIKRQLVETRQITKHVAQILDSRMNTKYDENDKLIREVKVITLKSKL | |
| | VSDFRKDFQFYKVREINNYHHAHDAYLNAVVGTALIKKYPKLESEFVYGDYKVYDVRKM | |
| | IAKSEQEIGKATAKYFFYSNIMNFFKTEITLANGEIRKRPLIETNGETGEIVWDKGRDF | |
| | ATVRKVLSMPQVNIVKKTEVQTGGFSKESILPKRNSDKLIARKKDWDPKKYGGFDSPTV | |
| | AYSVLVVAKVEKGKSKKLKSVKELLGITIMERSSFEKNPIDFLEAKGYKEVKKDLIIKL | |
| | PKYSLFELENGRKRMLASAGELQKGNELALPSKYVNFLYLASHYEKLKGSPEDNEQKQL | |
| | FVEQHKHYLDEIIEQISEFSKRVILADANLDKVLSAYNKHRDKPIREQAENIIHLFTLT | |
| | NLGAPAAFKYFDTTIDRKRYTSTKEVLDATLIHQSITGLYETRIDLSQLGGD [symbol] | |
| | [symbol] [symbol] [symbol] [symbol] [symbol] | |
| | [symbol] [symbol] [symbol] [symbol] [symbol] | |
| | [symbol] [symbol] [symbol] [symbol] [symbol] | |
| | [symbol] KRTADGSEFEPKKKRKV | |
| EA-BE4 | MKRTADGSEFESPKKKRKVSSETGPVAVDPTLRRRIEPHEFEVFFDPRELRKETCLLYE | 3201 |
| Cas9 = SpCas9 | INWGGREAIWRHTSQNTNKHVEVNFIEKFTTERYFCPNTRCSITWFLSWSPCGECSRAI | |
| | TEFLSRYPHVTLFIYIARLYHHADPRNRQGLRDLISSGVTIQIMTEQESGYCWRNFVNY | |
| | SPSNEAHWPRYPHLWVRLYVLELYCIILGLPPCLNILRRKQPQLTFFTIALQSCHYQRL | |
| | PPHILWATGLKSGGSSGGSSGSETPGTSESATPESSGGSSGGDKKYSIGLAIGTNSVGW | |
| | AVITDEYKVPSKKFKVLGNTDRHSIKKNLIGALLFDSGETAEATRLKRTARRRYTRRKN | |
| | RICYLQEIFSNEMAKVDDSFFHRLEESFLVEEDKKHERHPIFGNIVDEVAYHEKYPTIY | |
| | HLRKKLVDSTDKADLRLIYLALAHMIKFRGHFLIEGDLNPDNSDVDKLFIQLVQTYNQL | |
| | FEENPINASGVDAKAILSARLSKSRRLENLIAQLPGEKKNGLFGNLIALSLGLTPNFKS | |
| | NFDLAEDAKLQLSKDTYDDDLDNLLAQIGDQYADLFLAAKNLSDAILLSDILRVNTEIT | |
| | KAPLSASMIKRYDEHHQDLTLLKALVRQQLPEKYKEIFFDQSKNGYAGYIDGGASQEEF | |
| | YKFIKPILEKMDGTEELLVKLNREDLLRKQRTFDNGSIPHQIHLGELHAILRRQEDFYP | |
| | FLKDNREKIEKILTFRIPYYVGPLARGNSRFAWMTRKSEETITPWNFEEVVDKGASAQS | |
| | FIERMTNFDKNLPNEKVLPKHSLLYEYFTVYNELTKVKYVTEGMRKPAFLSGEQKKAIV | |
| | DLLFKTNRKVTVKQLKEDYFKKIECFDSVEISGVEDRFNASLGTYHDLLKIIKDKDFLD | |
| | NEENEDILEDIVLTLTLFEDREMIEERLKTYAHLFDDKVMKQLKRRRYTGWGRLSRKLI | |
| | NGIRDKQSGKTILDFLKSDGFANRNFMQLIHDDSLTFKEDIQKAQVSGQGDSLHEHIAN | |
| | LAGSPAIKKGILQTVKVVDELVKVMGRHKPENIVIEMARENQTTQKGQKNSRERMKRIE | |
| | EGIKELGSQILKEHPVENTQLQNEKLYLYYLQNGRDMYVDQELDINRLSDYDVDHIVPQ | |
| | SFLKDDSIDNKVLTRSDKNRGKSDNVPSEEVVKKMKNYWRQLLNAKLITQRKFDNLTKA | |
| | ERGGLSELDKAGFIKRQLVETRQITKHVAQILDSRMNTKYDENDKLIREVKVITLKSKL | |
| | VSDFRKDFQFYKVREINNYHHAHDAYLNAVVGTALIKKYPKLESEFVYGDYKVYDVRKM | |
| | IAKSEQEIGKATAKYFFYSNIMNFFKTEITLANGEIRKRPLIETNGETGEIVWDKGRDF | |
| | ATVRKVLSMPQVNIVKKTEVQTGGFSKESILPKRNSDKLIARKKDWDPKKYGGFDSPTV | |
| | AYSVLVVAKVEKGKSKKLKSVKELLGITIMERSSFEKNPIDFLEAKGYKEVKKDLIIKL | |
| | PKYSLFELENGRKRMLASAGELQKGNELALPSKYVNFLYLASHYEKLKGSPEDNEQKQL | |
| | FVEQHKHYLDEIIEQISEFSKRVILADANLDKVLSAYNKHRDKPIREQAENIIHLFTLT | |
| | NLGAPAAFKYFDTTIDRKRYTSTKEVLDATLIHQSITGLYETRIDLSQLGGDS [symbol] | |
| | [symbol] [symbol] [symbol] [symbol] | |
| | [symbol] [symbol] [symbol] [symbol] | |
| | [symbol] [symbol] [symbol] [symbol] | |
| | [symbol] KRTADGSEFEPKKKRKV | |
| AID-BE4 | MKRTADGSEFESPKKKRKVDSLLMNRRKFLYQFKNVRWAKGRRETYLCYVVKRRDSATS | 3202 |
| Cas9 = SpCas9 | FSLDFGYLRNKNGCHVELLFLRYISDWDLDPGRCYRVTWFTSWSPCYDCARHVADFLRG | |
| | NPNLSLRIFTARLYFCEDRKAEPEGLRRLHRAGVQIAIMTFKDYFYCWNTFVENHERTF | |
| | KAWEGLHENSVRLSRQLRRILLPLYEVDDLRDAFRTLGLSGGSSGGSSGSETPGTSESA | |
| | TPESSGGSSGGDKKYSIGLAIGTNSVGWAVITDEYKVPSKKFKVLGNTDRHSIKKNLIG | |
| | ALLFDSGETAEATRLKRTARRRYTRRKNRICYLQEIFSNEMAKVDDSFFHRLEESFLVE | |
| | EDKKHERHPIFGNIVDEVAYHEKYPTIYHLRKKLVDSTDKADLRLIYLALAHMIKFRGH | |
| | FLIEGDLNPDNSDVDKLFIQLVQTYNQLFEENPINASGVDAKAILSARLSKSRRLENLI | |
| | AQLPGEKKNGLFGNLIALSLGLTPNFKSNFDLAEDAKLQLSKDTYDDDLDNLLAQIGDQ | |
| | YADLFLAAKNLSDAILLSDILRVNTEITKAPLSASMIKRYDEHHQDLTLLKALVRQQLP | |
| | EKYKEIFFDQSKNGYAGYIDGGASQEEFYKFIKPILEKMDGTEELLVKLNREDLLRKQR | |
| | TFDNGSIPHQIHLGELHAILRRQEDFYPFLKDNREKIEKILTFRIPYYVGPLARGNSRF | |
| | AWMTRKSEETITPWNFEEVVDKGASAQSFIERMTNFDKNLPNEKVLPKHSLLYEYFTVY | |
| | NELTKVKYVTEGMRKPAFLSGEQKKAIVDLLFKTNRKVTVKQLKEDYFKKIECFDSVEI | |
| | SGVEDRFNASLGTYHDLLKIIKDKDFLDNEENEDILEDIVLTLTLFEDREMIEERLKTY | |
| | AHLFDDKVMKQLKRRRYTGWGRLSRKLINGIRDKQSGKTILDFLKSDGFANRNFMQLIH | |
| | DDSLTFKEDIQKAQVSGQGDSLHEHIANLAGSPAIKKGILQTVKVVDELVKVMGRHKPE | |
| | NIVIEMARENQTTQKGQKNSRERMKRIEEGIKELGSQILKEHPVENTQLQNEKLYLYYL | |
| | QNGRDMYVDQELDINRLSDYDVDHIVPQSFLKDDSIDNKVLTRSDKNRGKSDNVPSEEV | |
| | VKKMKNYWRQLLNAKLITQRKFDNLTKAERGGLSELDKAGFIKRQLVETRQITKHVAQI | |
| | LDSRMNTKYDENDKLIREVKVITLKSKLVSDFRKDFQFYKVREINNYHHAHDAYLNAVV | |
| | GTALIKKYPKLESEFVYGDYKVYDVRKMIAKSEQEIGKATAKYFFYSNIMNFFKTEITL | |
| | ANGEIRKRPLIETNGETGEIVWDKGRDFATVRKVLSMPQVNIVKKTEVQTGGFSKESIL | |
| | PKRNSDKLIARKKDWDPKKYGGFDSPTVAYSVLVVAKVEKGKSKKLKSVKELLGITIME | |
| | RSSFEKNPIDFLEAKGYKEVKKDLIIKLPKYSLFELENGRKRMLASAGELQKGNELALP | |
| | SKYVNFLYLASHYEKLKGSPEDNEQKQLFVEQHKHYLDEIIEQISEFSKRVILADANLD | |
| | KVLSAYNKHRDKPIREQAENIIHLFTLTNLGAPAAFKYFDTTIDRKRYTSTKEVLDATL | |
| | IHQSITGLYETRIDLSQLGGD [symbol] [symbol] [symbol] | |
| | [symbol] [symbol] [symbol] [symbol] [symbol] | |
| | [symbol] [symbol] [symbol] [symbol] [symbol] | |
| | [symbol] [symbol] [symbol] KRTADGSEFEPKKKRKV | |
| CDA-BE4 (or CDA1- | MKRTADGSEFESPKKKRKVTDAEYVRIHEKLDIYTFKKQFFNNKKSVSHRCYVLFELKR | 3203 |
| BE4max) | RGERRACFWGYAVNKPQSGTERGIHAEIFSIRKVEEYLRDNPGQFTINWYSSWSPCADC | |
| Cas9 = SpCas9 | AEKILEWYNQELRGNGHTLKIWACKLYYEKNARNQIGLWNLRDNGVGLNVMVSEHYQCC | |
| | RKIFIQSSHNQLNENRWLEKTLKRAEKRRSELSIMIQVKILHTTKSPAVSGGSSGGSSG | |
| | SETPGTSESATPESSGGSSGGDKKYSIGLAIGTNSVGWAVITDEYKVPSKKFKVLGNTD | |
| | RHSIKKNLIGALLFDSGETAEATRLKRTARRRYTRRKNRICYLQEIFSNEMAKVDDSFF | |
| | HRLEESFLVEEDKKHERHPIFGNIVDEVAYHEKYPTIYHLRKKLVDSTDKADLRLIYLA | |
| | LAHMIKFRGHFLIEGDLNPDNSDVDKLFIQLVQTYNQLFEENPINASGVDAKAILSARL | |
| | SKSRRLENLIAQLPGEKKNGLFGNLIALSLGLTPNFKSNFDLAEDAKLQLSKDTYDDDL | |
| | DNLLAQIGDQYADLFLAAKNLSDAILLSDILRVNTEITKAPLSASMIKRYDEHHQDLTL | |
| | LKALVRQQLPEKYKEIFFDQSKNGYAGYIDGGASQEEFYKFIKPILEKMDGTEELLVKL | |
| | NREDLLRKQRTFDNGSIPHQIHLGELHAILRRQEDFYPFLKDNREKIEKILTFRIPYYV | |
| | GPLARGNSRFAWMTRKSEETITPWNFEEVVDKGASAQSFIERMTNFDKNLPNEKVLPKH | |
| | SLLYEYFTVYNELTKVKYVTEGMRKPAFLSGEQKKAIVDLLFKTNRKVTVKQLKEDYFK | |
| | KIECFDSVEISGVEDRFNASLGTYHDLLKIIKDKDFLDNEENEDILEDIVLTLTLFEDR | |
| | EMIEERLKTYAHLFDDKVMKQLKRRRYTGWGRLSRKLINGIRDKQSGKTILDFLKSDGF | |
| | ANRNFMQLIHDDSLTFKEDIQKAQVSGQGDSLHEHIANLAGSPAIKKGILQTVKVVDEL | |
| | VKVMGRHKPENIVIEMARENQTTQKGQKNSRERMKRIEEGIKELGSQILKEHPVENTQL | |
| | QNEKLYLYYLQNGRDMYVDQELDINRLSDYDVDHIVPQSFLKDDSIDNKVLTRSDKNRG | |
| | KSDNVPSEEVVKKMKNYWRQLLNAKLITQRKFDNLTKAERGGLSELDKAGFIKRQLVET | |
| | RQITKHVAQILDSRMNTKYDENDKLIREVKVITLKSKLVSDFRKDFQFYKVREINNYHH | |
| | AHDAYLNAVVGTALIKKYPKLESEFVYGDYKVYDVRKMIAKSEQEIGKATAKYFFYSNI | |
| | MNFFKTEITLANGEIRKRPLIETNGETGEIVWDKGRDFATVRKVLSMPQVNIVKKTEVQ | |
| | TGGFSKESILPKRNSDKLIARKKDWDPKKYGGFDSPTVAYSVLVVAKVEKGKSKKLKSV | |
| | KELLGITIMERSSFEKNPIDFLEAKGYKEVKKDLIIKLPKYSLFELENGRKRMLASAGE | |
| | LQKGNELALPSKYVNFLYLASHYEKLKGSPEDNEQKQLFVEQHKHYLDEIIEQISEFSK | |
| | RVILADANLDKVLSAYNKHRDKPIREQAENIIHLFTLTNLGAPAAFKYFDTTIDRKRYT | |
| | STKEVLDATLIHQSITGLYETRIDLSQLGGDS [symbol] [symbol] | |
| | [symbol] [symbol] [symbol] [symbol] | |
| | [symbol] [symbol] [symbol] [symbol] [symbol] | |
| | [symbol] [symbol] [symbol] [symbol] | |
| | [symbol] KRTADGSEFEPKKKRKV | |
| evoA-BE4 (or | MKRTADGSEFESPKKKRKVSKTGPVAVDPTLRRRIEPHEFEVFFDPRELRKETCLLYEI | 3204 |
| evoAPOBEC1-BE4max) | NWGGRHSIWRHTSQNTNKHVEVNFIEKFTTERYFCPNTRCSITWFLSWSPCGECSRAIT | |
| Cas9 = SpCas9 | EFLSRYPNVTLFIYIARLYHLANPRNRQGLRDLISSGVTIQIMTEQESGYCWHNFVNYS | |
| | PSNESHWPRYPHLWVRLYVLELYCIILGLPPCLNILRRKQSQLTSFTIALQSCHYQRLP | |
| | PHILWATGLKSGGSSGGSSGSETPGTSESATPESSGGSSGGDKKYSIGLAIGTNSVGWA | |
| | VITDEYKVPSKKFKVLGNTDRHSIKKNLIGALLFDSGETAEATRLKRTARRRYTRRKNR | |
| | ICYLQEIFSNEMAKVDDSFFHRLEESFLVEEDKKHERHPIFGNIVDEVAYHEKYPTIYH | |
| | LRKKLVDSTDKADLRLIYLALAHMIKFRGHFLIEGDLNPDNSDVDKLFIQLVQTYNQLF | |
| | EENPINASGVDAKAILSARLSKSRRLENLIAQLPGEKKNGLFGNLIALSLGLTPNFKSN | |
| | FDLAEDAKLQLSKDTYDDDLDNLLAQIGDQYADLFLAAKNLSDAILLSDILRVNTEITK | |
| | APLSASMIKRYDEHHQDLTLLKALVRQQLPEKYKEIFFDQSKNGYAGYIDGGASQEEFY | |
| | KFIKPILEKMDGTEELLVKLNREDLLRKQRTFDNGSIPHQIHLGELHAILRRQEDFYPF | |
| | LKDNREKIEKILTFRIPYYVGPLARGNSRFAWMTRKSEETITPWNFEEVVDKGASAQSF | |
| | IERMTNFDKNLPNEKVLPKHSLLYEYFTVYNELTKVKYVTEGMRKPAFLSGEQKKAIVD | |
| | LLFKTNRKVTVKQLKEDYFKKIECFDSVEISGVEDRFNASLGTYHDLLKIIKDKDFLDN | |
| | EENEDILEDIVLTLTLFEDREMIEERLKTYAHLFDDKVMKQLKRRRYTGWGRLSRKLIN | |
| | GIRDKQSGKTILDFLKSDGFANRNFMQLIHDDSLTFKEDIQKAQVSGQGDSLHEHIANL | |
| | AGSPAIKKGILQTVKVVDELVKVMGRHKPENIVIEMARENQTTQKGQKNSRERMKRIEE | |
| | GIKELGSQILKEHPVENTQLQNEKLYLYYLQNGRDMYVDQELDINRLSDYDVDHIVPQS | |
| | FLKDDSIDNKVLTRSDKNRGKSDNVPSEEVVKKMKNYWRQLLNAKLITQRKFDNLTKAE | |
| | RGGLSELDKAGFIKRQLVETRQITKHVAQILDSRMNTKYDENDKLIREVKVITLKSKLV | |
| | SDFRKDFQFYKVREINNYHHAHDAYLNAVVGTALIKKYPKLESEFVYGDYKVYDVRKMI | |
| | AKSEQEIGKATAKYFFYSNIMNFFKTEITLANGEIRKRPLIETNGETGEIVWDKGRDFA | |
| | TVRKVLSMPQVNIVKKTEVQTGGFSKESILPKRNSDKLIARKKDWDPKKYGGFDSPTVA | |
| | YSVLVVAKVEKGKSKKLKSVKELLGITIMERSSFEKNPIDFLEAKGYKEVKKDLIIKLP | |
| | KYSLFELENGRKRMLASAGELQKGNELALPSKYVNFLYLASHYEKLKGSPEDNEQKQLF | |
| | VEQHKHYLDEIIEQISEFSKRVILADANLDKVLSAYNKHRDKPIREQAENIIHLFTLTN | |
| | LGAPAAFKYFDTTIDRKRYTSTKEVLDATLIHQSITGLYETRIDLSQLGGD [symbol] | |
| | [symbol] [symbol] [symbol] [symbol] | |
| | [symbol] [symbol] [symbol] [symbol] | |
| | [symbol] [symbol] [symbol] [symbol] | |
| | [symbol] KRTADGSEFEPKKKRKV | |
| eA3A-BE4 (or APOBEC3A) | MKRTADGSEFESPKKKRKVEASPASGPRHLMDPHIFTSNFNNGIGRHKTYLCYEVERLD | 3205 |
| Cas9 = SpCas9 | NGTSVKMDQHRGFLHGQAKNLLCGFYGRHAELRFLDLVPSLQLDPAQIYRVTWFISWSP | |
| | CFSWGCAGEVRAFLQENTHVRLRIFAARIYDYDPLYKEALQMLRDAGAQVSIMTYDEFK | |
| | HCWDTFVDHQGCPFQPWDGLDEHSQALSGRLRAILQNQGNSGGSSGGSSGSETPGTSES | |
| | ATPESSGGSSGGDKKYSIGLAIGTNSVGWAVITDEYKVPSKKFKVLGNTDRHSIKKNLI | |
| | GALLFDSGETAEATRLKRTARRRYTRRKNRICYLQEIFSNEMAKVDDSFFHRLEESFLV | |
| | EEDKKHERHPIFGNIVDEVAYHEKYPTIYHLRKKLVDSTDKADLRLIYLALAHMIKFRG | |
| | HFLIEGDLNPDNSDVDKLFIQLVQTYNQLFEENPINASGVDAKAILSARLSKSRRLENL | |
| | IAQLPGEKKNGLFGNLIALSLGLTPNFKSNFDLAEDAKLQLSKDTYDDDLDNLLAQIGD | |
| | QYADLFLAAKNLSDAILLSDILRVNTEITKAPLSASMIKRYDEHHQDLTLLKALVRQQL | |
| | PEKYKEIFFDQSKNGYAGYIDGGASQEEFYKFIKPILEKMDGTEELLVKLNREDLLRKQ | |
| | RTFDNGSIPHQIHLGELHAILRRQEDFYPFLKDNREKIEKILTFRIPYYVGPLARGNSR | |
| | FAWMTRKSEETITPWNFEEVVDKGASAQSFIERMTNFDKNLPNEKVLPKHSLLYEYFTV | |
| | YNELTKVKYVTEGMRKPAFLSGEQKKAIVDLLFKTNRKVTVKQLKEDYFKKIECFDSVE | |
| | ISGVEDRFNASLGTYHDLLKIIKDKDFLDNEENEDILEDIVLTLTLFEDREMIEERLKT | |
| | YAHLFDDKVMKQLKRRRYTGWGRLSRKLINGIRDKQSGKTILDFLKSDGFANRNFMQLI | |
| | HDDSLTFKEDIQKAQVSGQGDSLHEHIANLAGSPAIKKGILQTVKVVDELVKVMGRHKP | |
| | ENIVIEMARENQTTQKGQKNSRERMKRIEEGIKELGSQILKEHPVENTQLQNEKLYLYY | |
| | LQNGRDMYVDQELDINRLSDYDVDHIVPQSFLKDDSIDNKVLTRSDKNRGKSDNVPSEE | |
| | VVKKMKNYWRQLLNAKLITQRKFDNLTKAERGGLSELDKAGFIKRQLVETRQITKHVAQ | |
| | ILDSRMNTKYDENDKLIREVKVITLKSKLVSDFRKDFQFYKVREINNYHHAHDAYLNAV | |
| | VGTALIKKYPKLESEFVYGDYKVYDVRKMIAKSEQEIGKATAKYFFYSNIMNFFKTEIT | |
| | LANGEIRKRPLIETNGETGEIVWDKGRDFATVRKVLSMPQVNIVKKTEVQTGGFSKESI | |
| | LPKRNSDKLIARKKDWDPKKYGGFDSPTVAYSVLVVAKVEKGKSKKLKSVKELLGITIM | |
| | ERSSFEKNPIDFLEAKGYKEVKKDLIIKLPKYSLFELENGRKRMLASAGELQKGNELAL | |
| | PSKYVNFLYLASHYEKLKGSPEDNEQKQLFVEQHKHYLDEIIEQISEFSKRVILADANL | |
| | DKVLSAYNKHRDKPIREQAENIIHLFTLTNLGAPAAFKYFDTTIDRKRYTSTKEVLDAT | |
| | LIHQSITGLYETRIDLSQLGGD [symbol] [symbol] | |
| | [symbol] [symbol] [symbol] [symbol] | |
| | [symbol] [symbol] [symbol] | |
| | [symbol] [symbol] [symbol] KRTADGSEFEPKKKRKV | |
| eA3A-T31A | MKRTADGSEFESPKKKRKVEASPASGPRHLMDPHIFTSNFNNGIGRHKAYLCYEVERLD | 3206 |
| Cas9 = SpCas9 | NGTSVKMDQHRGFLHGQAKNLLCGFYGRHAELRFLDLVPSLQLDPAQIYRVTWFISWSP | |
| | CFSWGCAGEVRAFLQENTHVRLRIFAARIYDYDPLYKEALQMLRDAGAQVSIMTYDEFK | |
| | HCWDTFVDHQGCPFQPWDGLDEHSQALSGRLRAILQNQGNSGGSSGGSSGSETPGTSES | |
| | ATPESSGGSSGGDKKYSIGLAIGTNSVGWAVITDEYKVPSKKFKVLGNTDRHSIKKNLI | |
| | GALLFDSGETAEATRLKRTARRRYTRRKNRICYLQEIFSNEMAKVDDSFFHRLEESFLV | |
| | EEDKKHERHPIFGNIVDEVAYHEKYPTIYHLRKKLVDSTDKADLRLIYLALAHMIKFRG | |
| | HFLIEGDLNPDNSDVDKLFIQLVQTYNQLFEENPINASGVDAKAILSARLSKSRRLENL | |
| | IAQLPGEKKNGLFGNLIALSLGLTPNFKSNFDLAEDAKLQLSKDTYDDDLDNLLAQIGD | |
| | QYADLFLAAKNLSDAILLSDILRVNTEITKAPLSASMIKRYDEHHQDLTLLKALVRQQL | |
| | PEKYKEIFFDQSKNGYAGYIDGGASQEEFYKFIKPILEKMDGTEELLVKLNREDLLRKQ | |
| | RTFDNGSIPHQIHLGELHAILRRQEDFYPFLKDNREKIEKILTFRIPYYVGPLARGNSR | |
| | FAWMTRKSEETITPWNFEEVVDKGASAQSFIERMTNFDKNLPNEKVLPKHSLLYEYFTV | |
| | YNELTKVKYVTEGMRKPAFLSGEQKKAIVDLLFKTNRKVTVKQLKEDYFKKIECFDSVE | |
| | ISGVEDRFNASLGTYHDLLKIIKDKDFLDNEENEDILEDIVLTLTLFEDREMIEERLKT | |
| | YAHLFDDKVMKQLKRRRYTGWGRLSRKLINGIRDKQSGKTILDFLKSDGFANRNFMQLI | |
| | HDDSLTFKEDIQKAQVSGQGDSLHEHIANLAGSPAIKKGILQTVKVVDELVKVMGRHKP | |
| | ENIVIEMARENQTTQKGQKNSRERMKRIEEGIKELGSQILKEHPVENTQLQNEKLYLYY | |
| | LQNGRDMYVDQELDINRLSDYDVDHIVPQSFLKDDSIDNKVLTRSDKNRGKSDNVPSEE | |
| | VVKKMKNYWRQLLNAKLITQRKFDNLTKAERGGLSELDKAGFIKRQLVETRQITKHVAQ | |
| | ILDSRMNTKYDENDKLIREVKVITLKSKLVSDFRKDFQFYKVREINNYHHAHDAYLNAV | |
| | VGTALIKKYPKLESEFVYGDYKVYDVRKMIAKSEQEIGKATAKYFFYSNIMNFFKTEIT | |
| | LANGEIRKRPLIETNGETGEIVWDKGRDFATVRKVLSMPQVNIVKKTEVQTGGFSKESI | |
| | LPKRNSDKLIARKKDWDPKKYGGFDSPTVAYSVLVVAKVEKGKSKKLKSVKELLGITIM | |
| | ERSSFEKNPIDFLEAKGYKEVKKDLIIKLPKYSLFELENGRKRMLASAGELQKGNELAL | |
| | PSKYVNFLYLASHYEKLKGSPEDNEQKQLFVEQHKHYLDEIIEQISEFSKRVILADANL | |
| | DKVLSAYNKHRDKPIREQAENIIHLFTLTNLGAPAAFKYFDTTIDRKRYTSTKEVLDAT | |
| | LIHQSITGLYETRIDLSQLGGD [symbol] [symbol] | |
| | [symbol] [symbol] [symbol] [symbol] | |
| | [symbol] [symbol] [symbol] [symbol] | |
| | [symbol] [symbol] KRTADGSEFEPKKKRKV | |
| eA3A-BE5 | MKRTADGSEFESPKKKRKVEASPASGPRHLMDPHIFTSNFNNGIGRHKTYLCYEVERLD | 3207 |
| Cas9 = SpCas9 | NGDAVKMDQHRGFLHGQAKNLLCGFYGRHAELRFLDLVPSLQLDPAQIYRVTWFISWSP | |
| | CFSWGCAGEVRAFLQENTHVRLRIFAARIYDYDPLYKEALQMLRDAGAQVSIMTYDEFK | |
| | HCWDTFVDHQGCPFQPWDGLDEHSQALSGRLRAILQNQGNSGGSSGGSSGSETPGTSES | |
| | ATPESSGGSSGGDKKYSIGLAIGTNSVGWAVITDEYKVPSKKFKVLGNTDRHSIKKNLI | |
| | GALLFDSGETAEATRLKRTARRRYTRRKNRICYLQEIFSNEMAKVDDSFFHRLEESFLV | |
| | EEDKKHERHPIFGNIVDEVAYHEKYPTIYHLRKKLVDSTDKADLRLIYLALAHMIKFRG | |
| | HFLIEGDLNPDNSDVDKLFIQLVQTYNQLFEENPINASGVDAKAILSARLSKSRRLENL | |
| | IAQLPGEKKNGLFGNLIALSLGLTPNFKSNFDLAEDAKLQLSKDTYDDDLDNLLAQIGD | |
| | QYADLFLAAKNLSDAILLSDILRVNTEITKAPLSASMIKRYDEHHQDLTLLKALVRQQL | |
| | PEKYKEIFFDQSKNGYAGYIDGGASQEEFYKFIKPILEKMDGTEELLVKLNREDLLRKQ | |
| | RTFDNGSIPHQIHLGELHAILRRQEDFYPFLKDNREKIEKILTFRIPYYVGPLARGNSR | |
| | FAWMTRKSEETITPWNFEEVVDKGASAQSFIERMTNFDKNLPNEKVLPKHSLLYEYFTV | |
| | YNELTKVKYVTEGMRKPAFLSGEQKKAIVDLLFKTNRKVTVKQLKEDYFKKIECFDSVE | |
| | ISGVEDRFNASLGTYHDLLKIIKDKDFLDNEENEDILEDIVLTLTLFEDREMIEERLKT | |
| | YAHLFDDKVMKQLKRRRYTGWGRLSRKLINGIRDKQSGKTILDFLKSDGFANRNFMQLI | |
| | HDDSLTFKEDIQKAQVSGQGDSLHEHIANLAGSPAIKKGILQTVKVVDELVKVMGRHKP | |
| | ENIVIEMARENQTTQKGQKNSRERMKRIEEGIKELGSQILKEHPVENTQLQNEKLYLYY | |
| | LQNGRDMYVDQELDINRLSDYDVDHIVPQSFLKDDSIDNKVLTRSDKNRGKSDNVPSEE | |
| | VVKKMKNYWRQLLNAKLITQRKFDNLTKAERGGLSELDKAGFIKRQLVETRQITKHVAQ | |
| | ILDSRMNTKYDENDKLIREVKVITLKSKLVSDFRKDFQFYKVREINNYHHAHDAYLNAV | |
| | VGTALIKKYPKLESEFVYGDYKVYDVRKMIAKSEQEIGKATAKYFFYSNIMNFFKTEIT | |
| | LANGEIRKRPLIETNGETGEIVWDKGRDFATVRKVLSMPQVNIVKKTEVQTGGFSKESI | |
| | LPKRNSDKLIARKKDWDPKKYGGFDSPTVAYSVLVVAKVEKGKSKKLKSVKELLGITIM | |
| | ERSSFEKNPIDFLEAKGYKEVKKDLIIKLPKYSLFELENGRKRMLASAGELQKGNELAL | |
| | PSKYVNFLYLASHYEKLKGSPEDNEQKQLFVEQHKHYLDEIIEQISEFSKRVILADANL | |
| | DKVLSAYNKHRDKPIREQAENIIHLFTLTNLGAPAAFKYFDTTIDRKRYTSTKEVLDAT | |
| | LIHQSITGLYETRIDLSQLGGD [symbol] [symbol] | |
| | [symbol] [symbol] [symbol] [symbol] | |
| | [symbol] [symbol] [symbol] [symbol] | |
| | [symbol] [symbol] KRTADGSEFEPKKKRKV | |
| BE4-CP1028 | MKRTADGSEFESPKKKRKVSSETGPVAVDPTLRRRIEPHEFEVFFDPRELRKETCLLYE | 3208 |
| Cas9 = Cas9 CP1028 | INWGGRHSIWRHTSQNTNKHVEVNFIEKFTTERYFCPNTRCSITWFLSWSPCGECSRAI | |
| | TEFLSRYPHVTLFIYIARLYHHADPRNRQGLRDLISSGVTIQIMTEQESGYCWRNFVNY | |
| | SPSNEAHWPRYPHLWVRLYVLELYCIILGLPPCLNILRRKQPQLTFFTIALQSCHYQRL | |
| | PPHILWATGLKSGGSSGGSSGSETPGTSESATPESSGGSSGGEIGKATAKYFFYSNIMN | |
| | FFKTEITLANGEIRKRPLIETNGETGEIVWDKGRDFATVRKVLSMPQVNIVKKTEVQTG | |
| | GFSKESILPKRNSDKLIARKKDWDPKKYGGFDSPTVAYSVLVVAKVEKGKSKKLKSVKE | |
| | LLGITIMERSSFEKNPIDFLEAKGYKEVKKDLIIKLPKYSLFELENGRKRMLASAGELQ | |
| | KGNELALPSKYVNFLYLASHYEKLKGSPEDNEQKQLFVEQHKHYLDEIIEQISEFSKRV | |
| | ILADANLDKVLSAYNKHRDKPIREQAENIIHLFTLTNLGAPAAFKYFDTTIDRKRYTST | |
| | KEVLDATLIHQSITGLYETRIDLSQLGGDGGSGGSGGSGGSGGSGGSGGMDKKYSIGLA | |
| | IGTNSVGWAVITDEYKVPSKKFKVLGNTDRHSIKKNLIGALLFDSGETAEATRLKRTAR | |
| | RRYTRRKNRICYLQEIFSNEMAKVDDSFFHRLEESFLVEEDKKHERHPIFGNIVDEVAY | |
| | HEKYPTIYHLRKKLVDSTDKADLRLIYLALAHMIKFRGHFLIEGDLNPDNSDVDKLFIQ | |
| | LVQTYNQLFEENPINASGVDAKAILSARLSKSRRLENLIAQLPGEKKNGLFGNLIALSL | |
| | GLTPNFKSNFDLAEDAKLQLSKDTYDDDLDNLLAQIGDQYADLFLAAKNLSDAILLSDI | |
| | LRVNTEITKAPLSASMIKRYDEHHQDLTLLKALVRQQLPEKYKEIFFDQSKNGYAGYID | |
| | GGASQEEFYKFIKPILEKMDGTEELLVKLNREDLLRKQRTFDNGSIPHQIHLGELHAIL | |
| | RRQEDFYPFLKDNREKIEKILTFRIPYYVGPLARGNSRFAWMTRKSEETITPWNFEEVV | |
| | DKGASAQSFIERMTNFDKNLPNEKVLPKHSLLYEYFTVYNELTKVKYVTEGMRKPAFLS | |
| | GEQKKAIVDLLFKTNRKVTVKQLKEDYFKKIECFDSVEISGVEDRFNASLGTYHDLLKI | |
| | IKDKDFLDNEENEDILEDIVLTLTLFEDREMIEERLKTYAHLFDDKVMKQLKRRRYTGW | |
| | GRLSRKLINGIRDKQSGKTILDFLKSDGFANRNFMQLIHDDSLTFKEDIQKAQVSGQGD | |
| | SLHEHIANLAGSPAIKKGILQTVKVVDELVKVMGRHKPENIVIEMARENQTTQKGQKNS | |
| | RERMKRIEEGIKELGSQILKEHPVENTQLQNEKLYLYYLQNGRDMYVDQELDINRLSDY | |
| | DVDHIVPQSFLKDDSIDNKVLTRSDKNRGKSDNVPSEEVVKKMKNYWRQLLNAKLITQR | |
| | KFDNLTKAERGGLSELDKAGFIKRQLVETRQITKHVAQILDSRMNTKYDENDKLIREVK | |
| | VITLKSKLVSDFRKDFQFYKVREINNYHHAHDAYLNAVVGTALIKKYPKLESEFVYGDY | |
| | KVYDVRKMIAK [symbol] [symbol] | |
| | [symbol] [symbol] [symbol] [symbol] | |
| | [symbol] [symbol] [symbol] [symbol] | |
| | [symbol] [symbol] KRTADGSEFEPKKKRKV | |
| BE4-Cas9-NG | MKRTADGSEFESPKKKRKVSSETGPVAVDPTLRRRIEPHEFEVFFDPRELRKETCLLYE | 3209 |
| Cas9 = Cas9 NG | INWGGRHSIWRHTSQNTNKHVEVNFIEKFTTERYFCPNTRCSITWFLSWSPCGECSRAI | |
| | TEFLSRYPHVTLFIYIARLYHHADPRNRQGLRDLISSGVTIQIMTEQESGYCWRNFVNY | |
| | SPSNEAHWPRYPHLWVRLYVLELYCIILGLPPCLNILRRKQPQLTFFTIALQSCHYQRL | |
| | PPHILWATGLKSGGSSGGSSGSETPGTSESATPESSGGSSGGDKKYSIGLAIGTNSVGW | |
| | AVITDEYKVPSKKFKVLGNTDRHSIKKNLIGALLFDSGETAEATRLKRTARRRYTRRKN | |
| | RICYLQEIFSNEMAKVDDSFFHRLEESFLVEEDKKHERHPIFGNIVDEVAYHEKYPTIY | |
| | HLRKKLVDSTDKADLRLIYLALAHMIKFRGHFLIEGDLNPDNSDVDKLFIQLVQTYNQL | |
| | FEENPINASGVDAKAILSARLSKSRRLENLIAQLPGEKKNGLFGNLIALSLGLTPNFKS | |
| | NFDLAEDAKLQLSKDTYDDDLDNLLAQIGDQYADLFLAAKNLSDAILLSDILRVNTEIT | |
| | KAPLSASMIKRYDEHHQDLTLLKALVRQQLPEKYKEIFFDQSKNGYAGYIDGGASQEEF | |
| | YKFIKPILEKMDGTEELLVKLNREDLLRKQRTFDNGSIPHQIHLGELHAILRRQEDFYP | |
| | FLKDNREKIEKILTFRIPYYVGPLARGNSRFAWMTRKSEETITPWNFEEVVDKGASAQS | |
| | FIERMTNFDKNLPNEKVLPKHSLLYEYFTVYNELTKVKYVTEGMRKPAFLSGEQKKAIV | |
| | DLLFKTNRKVTVKQLKEDYFKKIECFDSVEISGVEDRFNASLGTYHDLLKIIKDKDFLD | |
| | NEENEDILEDIVLTLTLFEDREMIEERLKTYAHLFDDKVMKQLKRRRYTGWGRLSRKLI | |
| | NGIRDKQSGKTILDFLKSDGFANRNFMQLIHDDSLTFKEDIQKAQVSGQGDSLHEHIAN | |
| | LAGSPAIKKGILQTVKVVDELVKVMGRHKPENIVIEMARENQTTQKGQKNSRERMKRIE | |
| | EGIKELGSQILKEHPVENTQLQNEKLYLYYLQNGRDMYVDQELDINRLSDYDVDHIVPQ | |
| | SFLKDDSIDNKVLTRSDKNRGKSDNVPSEEVVKKMKNYWRQLLNAKLITQRKFDNLTKA | |
| | ERGGLSELDKAGFIKRQLVETRQITKHVAQILDSRMNTKYDENDKLIREVKVITLKSKL | |
| | VSDFRKDFQFYKVREINNYHHAHDAYLNAVVGTALIKKYPKLESEFVYGDYKVYDVRKM | |
| | IAKSEQEIGKATAKYFFYSNIMNFFKTEITLANGEIRKRPLIETNGETGEIVWDKGRDF | |
| | ATVRKVLSMPQVNIVKKTEVQTGGFSKESIRPKRNSDKLIARKKDWDPKKYGGFVSPTV | |
| | AYSVLVVAKVEKGKSKKLKSVKELLGITIMERSSFEKNPIDFLEAKGYKEVKKDLIIKL | |
| | PKYSLFELENGRKRMLASARFLQKGNELALPSKYVNFLYLASHYEKLKGSPEDNEQKQL | |
| | FVEQHKHYLDEIIEQISEFSKRVILADANLDKVLSAYNKHRDKPIREQAENIIHLFTLT | |
| | NLGAPRAFKYFDTTIDRKVYRSTKEVLDATLIHQSITGLYETRIDLSQLGGD [symbol] | |
| | [symbol] [symbol] [symbol] [symbol] [symbol] | |
| | [symbol] [symbol] [symbol] [symbol] [symbol] | |
| | [symbol] [symbol] [symbol] [symbol] | |
| | [symbol] KRTADGSEFEPKKKRKV | |

Key:
NLS (N-terminal) Single underline
APOBEC 1 (BE4) Double underline
Linker Italic
SpCas9 Plain
Linker + 2xUGI Bold underline
NLS (C-terminal) Single underline + italic

The following ABEs were used to generate training data or the BE-Hive algorithm of Example 1. Each of the ABEs have the same architecture of [NLS]-[deaminase]-[Cas9]-[NLS] (which is the ABEmax architecture) and use the same adenine deaminase, ABE7.10, with either the SpCas9 or CP1041 circular permutant variant as the Cas9 component.

| | | SEQ ID |
|---|---|---|
| DESCRIPTION | SEQUENCE | NO: |
| ABEmax (or ABE) | MKRTADGSEFESPKKKRKVSEVEFSHEYWMRHALTLAKRAWDEREVPVGAVLVHNNRVI | 3210 |
| | GEGWNRPIGRHDPTAHAEIMALRQGGLVMQNYRLIDATLYVTLEPCVMCAGAMIHSRIG | |
| | RVVFGARDAKTGAAGSLMDVLHHPGMNHRVEITEGILADECAALLSDFFRMRRQEIKAQ | |
| | KKAQSSTDSGGSSGGSSGSETPGTSESATPESSGGSSGGSSEVEFSHEYWMRHALTLAK | |
| | RARDEREVPVGAVLVLNNRVIGEGWNRAIGLHDPTAHAEIMALRQGGLVMQNYRLIDAT | |
| | LYVTFEPCVMCAGAMIHSRIGRVVFGVRNAKTGAAGSLMDVLHYPGMNHRVEITEGILA | |
| | DECAALLCYFFRMPRQVFNAQKKAQSSTDSGGSSGGSSGSETPGTSESATPESSGGSSG | |
| | GSDKKYSIGLAIGTNSVGWAVITDEYKVPSKKFKVLGNTDRHSIKKNLIGALLFDSGET | |
| | AEATRLKRTARRRYTRRKNRICYLQEIFSNEMAKVDDSFFHRLEESFLVEEDKKHERHP | |
| | IFGNIVDEVAYHEKYPTIYHLRKKLVDSTDKADLRLIYLALAHMIKFRGHFLIEGDLNP | |
| | DNSDVDKLFIQLVQTYNQLFEENPINASGVDAKAILSARLSKSRRLENLIAQLPGEKKN | |
| | GLFGNLIALSLGLTPNFKSNFDLAEDAKLQLSKDTYDDDLDNLLAQIGDQYADLFLAAK | |
| | NLSDAILLSDILRVNTEITKAPLSASMIKRYDEHHQDLTLLKALVRQQLPEKYKEIFFD | |
| | QSKNGYAGYIDGGASQEEFYKFIKPILEKMDGTEELLVKLNREDLLRKQRTFDNGSIPH | |
| | QIHLGELHAILRRQEDFYPFLKDNREKIEKILTFRIPYYVGPLARGNSRFAWMTRKSEE | |
| | TITPWNFEEVVDKGASAQSFIERMTNFDKNLPNEKVLPKHSLLYEYFTVYNELTKVKYV | |
| | TEGMRKPAFLSGEQKKAIVDLLFKTNRKVTVKQLKEDYFKKIECFDSVEISGVEDRFNA | |
| | SLGTYHDLLKIIKDKDFLDNEENEDILEDIVLTLTLFEDREMIEERLKTYAHLFDDKVM | |
| | KQLKRRRYTGWGRLSRKLINGIRDKQSGKTILDFLKSDGFANRNFMQLIHDDSLTFKED | |
| | IQKAQVSGQGDSLHEHIANLAGSPAIKKGILQTVKVVDELVKVMGRHKPENIVIEMARE | |
| | NQTTQKGQKNSRERMKRIEEGIKELGSQILKEHPVENTQLQNEKLYLYYLQNGRDMYVD | |
| | QELDINRLSDYDVDHIVPQSFLKDDSIDNKVLTRSDKNRGKSDNVPSEEVVKKMKNYWR | |
| | QLLNAKLITQRKFDNLTKAERGGLSELDKAGFIKRQLVETRQITKHVAQILDSRMNTKY | |
| | DENDKLIREVKVITLKSKLVSDFRKDFQFYKVREINNYHHAHDAYLNAVVGTALIKKYP | |
| | KLESEFVYGDYKVYDVRKMIAKSEQEIGKATAKYFFYSNIMNFFKTEITLANGEIRKRP | |
| | LIETNGETGEIVWDKGRDFATVRKVLSMPQVNIVKKTEVQTGGFSKESILPKRNSDKLI | |
| | ARKKDWDPKKYGGFDSPTVAYSVLVVAKVEKGKSKKLKSVKELLGITIMERSSFEKNPI | |
| | DFLEAKGYKEVKKDLIIKLPKYSLFELENGRKRMLASAGELQKGNELALPSKYVNFLYL | |
| | ASHYEKLKGSPEDNEQKQLFVEQHKHYLDEIIEQISEFSKRVILADANLDKVLSAYNKH | |
| | RDKPIREQAENIIHLFTLTNLGAPAAFKYFDTTIDRKRYTSTKEVLDATLIHQSITGLY | |
| | ETRIDLSQLGGD [symbol] [symbol] [symbol] [symbol] | |
| | [symbol] [symbol] [symbol] [symbol] | |
| | [symbol] [symbol] [symbol] [symbol] | |
| | [symbol] [symbol] KRTADGSEFEPKKKRKV | |
| ABE-CP1041 (or ABE-CP) | MKRTADGSEFESPKKKRKVSEVEFSHEYWMRHALTLAKRAWDEREVPVGAVLVHNNRVI | 3211 |
| | GEGWNRPIGRHDPTAHAEIMALRQGGLVMQNYRLIDATLYVTLEPCVMCAGAMIHSRIG | |
| | RWFGARDAKTGAAGSLMDVLHHPGMNHRVEITEGILADECAALLSDFFRMRRQEIKAQ | |
| | KKAQSSTDSGGSSGGSSGSETPGTSESATPESSGGSSGGSSEVEFSHEYWMRHALTLAK | |
| | RARDEREVPVGAVLVLNNRVIGEGWNRAIGLHDPTAHAEIMALRQGGLVMQNYRLIDAT | |
| | LYVTFEPCVMCAGAMIHSRIGRVVFGVRNAKTGAAGSLMDVLHYPGMNHRVEITEGILA | |
| | DECAALLCYFFRMPRQVFNAQKKAQSSTDSGGSSGGSSGSETPGTSESATPESSGGSSG | |
| | GSNIMNFFKTEITLANGEIRKRPLIETNGETGEIVWDKGRDFATVRKVLSMPQVNIVKK | |
| | TEVQTGGFSKESILPKRNSDKLIARKKDWDPKKYGGFDSPTVAYSVLVVAKVEKGKSKK | |
| | LKSVKELLGITIMERSSFEKNPIDFLEAKGYKEVKKDLIIKLPKYSLFELENGRKRMLA | |
| | SAGELQKGNELALPSKYVNFLYLASHYEKLKGSPEDNEQKQLFVEQHKHYLDEIIEQIS | |
| | EFSKRVILADANLDKVLSAYNKHRDKPIREQAENIIHLFTLTNLGAPAAFKYFDTTIDR | |
| | KRYTSTKEVLDATLIHQSITGLYETRIDLSQLGGDGGSGGSGGSGGSGGSGGSGGDKKY | |
| | SIGLAIGTNSVGWAVITDEYKVPSKKFKVLGNTDRHSIKKNLIGALLFDSGETAEATRL | |
| | KRTARRRYTRRKNRICYLQEIFSNEMAKVDDSFFHRLEESFLVEEDKKHERHPIFGNIV | |
| | DEVAYHEKYPTIYHLRKKLVDSTDKADLRLIYLALAHMIKFRGHFLIEGDLNPDNSDVD | |
| | KLFIQLVQTYNQLFEENPINASGVDAKAILSARLSKSRRLENLIAQLPGEKKNGLFGNL | |
| | IALSLGLTPNFKSNFDLAEDAKLQLSKDTYDDDLDNLLAQIGDQYADLFLAAKNLSDAI | |
| | LLSDILRVNTEITKAPLSASMIKRYDEHHQDLTLLKALVRQQLPEKYKEIFFDQSKNGY | |
| | AGYIDGGASQEEFYKFIKPILEKMDGTEELLVKLNREDLLRKQRTFDNGSIPHQIHLGE | |
| | LHAILRRQEDFYPFLKDNREKIEKILTFRIPYYVGPLARGNSRFAWMTRKSEETITPWN | |
| | FEEVVDKGASAQSFIERMTNFDKNLPNEKVLPKHSLLYEYFTVYNELTKVKYVTEGMRK | |
| | PAFLSGEQKKAIVDLLFKTNRKVTVKQLKEDYFKKIECFDSVEISGVEDRFNASLGTYH | |
| | DLLKIIKDKDFLDNEENEDILEDIVLTLTLFEDREMIEERLKTYAHLFDDKVMKQLKRR | |
| | RYTGWGRLSRKLINGIRDKQSGKTILDFLKSDGFANRNFMQLIHDDSLTFKEDIQKAQV | |
| | SGQGDSLHEHIANLAGSPAIKKGILQTVKVVDELVKVMGRHKPENIVIEMARENQTTQK | |
| | GQKNSRERMKRIEEGIKELGSQILKEHPVENTQLQNEKLYLYYLQNGRDMYVDQELDIN | |
| | RLSDYDVDHIVPQSFLKDDSIDNKVLTRSDKNRGKSDNVPSEEVVKKMKNYWRQLLNAK | |
| | LITQRKFDNLTKAERGGLSELDKAGFIKRQLVETRQITKHVAQILDSRMNTKYDENDKL | |
| | IREVKVITLKSKLVSDFRKDFQFYKVREINNYHHAHDAYLNAVVGTALIKKYPKLESEF | |
| | VYGDYKVYDVRKMIAKSEQEIGKATAKYFFYS [symbol] [symbol] | |
| | [symbol] [symbol] [symbol] [symbol] | |
| | [symbol] [symbol] [symbol] [symbol] | |
| | [symbol] [symbol] [symbol] [symbol] | |
| | KRTADGSEFEPKKKRKV | |

Key:
NLS (N-terminal) Single underline
APOBEC 1 (BE4) Double underline
Linker Italic
SpCas9 Plain
inker + 2xUGI Bold underline
NLS (C-terminal) Single underline + italic

### Additional Exemplary ABEs

Some aspects of the disclosure provide base editors comprising a base editor comprising a napDNAbp domain (e.g., an nCas9 domain) and one or more adenosine deaminase domains (e.g., a heterodimer of adenosine deaminases). Such fusion proteins can be referred to as adenine base editors (ABEs). In some embodiments, the ABEs have reduced off-target effects. In some embodiments, the base editors comprise adenine base editors for multiplexing applications. In still other embodiments, the base editors comprise ancestrally reconstructed adenine base editors.

The present disclosure provides motifs of newly discovered mutations to TadA 7.10 (SEQ ID NO: 79) (the TadA* used in ABEmax) that yield adenosine deaminase variants and confer broader Cas compatibility to the deaminase. These motifs also confer reduced off-target effects, such as reduced RNA editing activity and off-target DNA editing activity, on the base editor. The base editors of the present disclosure comprise one or more of the disclosed adenosine deaminase variants. In other embodiments, the base editors may comprise one or more adenosine deaminases having two or more such substitutions in combination. In some embodiments, the base editors comprise adenosine deaminases comprising comprises a sequence with at least 80%, 85%, 90%, 95%, 98%, 99%, or 99.5% sequence identity to SEQ ID NO: 91 (TadA-8e).

Exemplary ABEs include, without limitation, the following fusion proteins (for the purposes of clarity, and wherein shown, the adenosine deaminase domain is shown in bold; mutations of the ecTadA deaminase domain are shown in bold underlining; the XTEN linker is shown in italics; the UGI/AAG/EndoV domains are shown in bold italics; and NLS is shown in underlined italics), and any base editors comprise sequences that are at least 85%, at least 90%, at least 95%, at least 98%, at least 99%, or at least 99.5% identical to any of the following amino acid sequences:

ecTadA(wt)-XTEN-nCas9-NLS
(SEQ ID NO: 174)
MSEVEFSHEYWMRHALTLAKRAWDEREVPVGAVLVHNNRVIGEGW
NRPIGRHDPTAHAEIMALRQGGLVMQNYRLIDATLYVTLEPCVMC
AGAMIHSRIGRVVFGARDAKTGAAGSLMDVLHHPGMNHRVEITEG
ILADECAALLSDFFRMRRQEIKAQKKAQSSTDSGSETPGTSESAT
PESDKKYSIGLAIGTNSVGWAVITDEYKVPSKKFKVLGNTDRHSI
KKNLIGALLFDSGETAEATRLKRTARRRYTRRKNRICYLQEIFSN
EMAKVDDSFFHRLEESFLVEEDKKHERHPIFGNIVDEVAYHEKYP
TIYHLRKKLVDSTDKADLRLIYLALAHMIKFRGHFLIEGDLNPDN
SDVDKLFIQLVQTYNQLFEENPINASGVDAKAILSARLSKSRRLE
NLIAQLPGEKKNGLFGNLIALSLGLTPNFKSNFDLAEDAKLQLSK
DTYDDDLDNLLAQIGDQYADLFLAAKNLSDAILLSDILRVNTEIT
KAPLSASMIKRYDEHHQDLTLLKALVRQQLPEKYKEIFFDQSKNG
YAGYIDGGASQEEFYKFIKPILEKMDGTEELLVKLNREDLLRKQR
TFDNGSIPHQIHLGELHAILRRQEDFYPFLKDNREKIEKILTFRI
PYYVGPLARGNSRFAWMTRKSEETITPWNFEEVVDKGASAQSFIE
RMTNFDKNLPNEKVLPKHSLLYEYFTVYNELTKVKYVTEGMRKPA
FLSGEQKKAIVDLLFKTNRKVTVKQLKEDYFKKIECFDSVEISGV
EDRFNASLGTYHDLLKIIKDKDFLDNEENEDILEDIVLTLTLFED
REMIEERLKTYAHLFDDKVMKQLKRRRYTGWGRLSRKLINGIRDK
QSGKTILDFLKSDGFANRNFMQLIHDDSLTFKEDIQKAQVSGQGD
SLHEHIANLAGSPAIKKGILQTVKVVDELVKVMGRHKPENIVIEM
ARENQTTQKGQKNSRERMKRIEEGIKELGSQILKEHPVENTQLQN
EKLYLYYLQNGRDMYVDQELDINRLSDYDVDHIVPQSFLKDDSID
NKVLTRSDKNRGKSDNVPSEEVVKKMKNYWRQLLNAKLITQRKFD
NLTKAERGGLSELDKAGFIKRQLVETRQITKHVAQILDSRMNTKY
DENDKLIREVKVITLKSKLVSDFRKDFQFYKVREINNYHHAHDAY
LNAVVGTALIKKYPKLESEFVYGDYKVYDVRKMIAKSEQEIGKAT
AKYFFYSNIMNFFKTEITLANGEIRKRPLIETNGETGEIVWDKGR
DFATVRKVLSMPQVNIVKKTEVQTGGFSKESILPKRNSDKLIARK
KDWDPKKYGGFDSPTVAYSVLVVAKVEKGKSKKLKSVKELLGITI
MERSSFEKNPIDFLEAKGYKEVKKDLIIKLPKYSLFELENGRKRM
LASAGELQKGNELALPSKYVNFLYLASHYEKLKGSPEDNEQKQLF
VEQHKHYLDEIIEQISEFSKRVILADANLDKVLSAYNKHRDKPIR
EQAENIIHLFTLTNLGAPAAFKYFDTTIDRKRYTSTKEVLDATLI
HQSITGLYETRIDLSQLGGDSGGSPKKKRKV
ecTadA(D108N)-XTEN-nCas9-NLS
(mammalian construct, active on DNA,
A to G editing):
(SEQ ID NO: 175)
MSEVEFSHEYWMRHALTLAKRAWDEREVPVGAVLVHNNRVIGEGW
NRPIGRHDPTAHAEIMALRQGGLVMQNYRLIDATLYVTLEPCVMC
AGAMIHSRIGRVVFGARNAKTGAAGSLMDVLHHPGMNHRVEITEG
ILADECAALLSDFFRMRRQEIKAQKKAQSSTDSGSETPGTSESAT
PESDKKYSIGLAIGTNSVGWAVITDEYKVPSKKFKVLGNTDRHSI
KKNLIGALLFDSGETAEATRLKRTARRRYTRRKNRICYLQEIFSN
EMAKVDDSFFHRLEESFLVEEDKKHERHPIFGNIVDEVAYHEKYP
TIYHLRKKLVDSTDKADLRLIYLALAHMIKFRGHFLIEGDLNPDN
SDVDKLFIQLVQTYNQLFEENPINASGVDAKAILSARLSKSRRLE
NLIAQLPGEKKNGLFGNLIALSLGLTPNFKSNFDLAEDAKLQLSK
DTYDDDLDNLLAQIGDQYADLFLAAKNLSDAILLSDILRVNTEIT
KAPLSASMIKRYDEHHQDLTLLKALVRQQLPEKYKEIFFDQSKNG
YAGYIDGGASQEEFYKFIKPILEKMDGTEELLVKLNREDLLRKQR
TFDNGSIPHQIHLGELHAILRRQEDFYPFLKDNREKIEKILTFRI
PYYVGPLARGNSRFAWMTRKSEETITPWNFEEVVDKGASAQSFIE
RMTNFDKNLPNEKVLPKHSLLYEYFTVYNELTKVKYVTEGMRKPA
FLSGEQKKAIVDLLFKTNRKVTVKQLKEDYFKKIECFDSVEISGV
EDRFNASLGTYHDLLKIIKDKDFLDNEENEDILEDIVLTLTLFED
REMIEERLKTYAHLFDDKVMKQLKRRRYTGWGRLSRKLINGIRDK
QSGKTILDFLKSDGFANRNFMQLIHDDSLTFKEDIQKAQVSGQGD
SLHEHIANLAGSPAIKKGILQTVKVVDELVKVMGRHKPENIVIEM
ARENQTTQKGQKNSRERMKRIEEGIKELGSQILKEHPVENTQLQN
EKLYLYYLQNGRDMYVDQELDINRLSDYDVDHIVPQSFLKDDSID
NKVLTRSDKNRGKSDNVPSEEVVKKMKNYWRQLLNAKLITQRKFD
NLTKAERGGLSELDKAGFIKRQLVETRQITKHVAQILDSRMNTKY
DENDKLIREVKVITLKSKLVSDFRKDFQFYKVREINNYHHAHDAY
LNAVVGTALIKKYPKLESEFVYGDYKVYDVRKMIAKSEQEIGKAT
AKYFFYSNIMNFFKTEITLANGEIRKRPLIETNGETGEIVWDKGR
DFATVRKVLSMPQVNIVKKTEVQTGGFSKESILPKRNSDKLIARK
KDWDPKKYGGFDSPTVAYSVLVVAKVEKGKSKKLKSVKELLGITI
MERSSFEKNPIDFLEAKGYKEVKKDLIIKLPKYSLFELENGRKRM
LASAGELQKGNELALPSKYVNFLYLASHYEKLKGSPEDNEQKQLF
VEQHKHYLDEIIEQISEFSKRVILADANLDKVLSAYNKHRDKPIR
EQAENIIHLFTLTNLGAPAAFKYFDTTIDRKRYTSTKEVLDATLI
HQSITGLYETRIDLSQLGGDSGGSPKKKRKV
ecTadA(D108G)-XTEN-nCas9-NLS
(mammalian construct, active on DNA,
A to G editing):
(SEQ ID NO: 176)
MSEVEFSHEYWMRHALTLAKRAWDEREVPVGAVLVHNNRVIGEGW
NRPIGRHDPTAHAEIMALRQGGLVMQNYRLIDATLYVTLEPCVMC
AGAMIHSRIGRVVFGARGAKTGAAGSLMDVLHHPGMNHRVEITEG
ILADECAALLSDFFRMRRQEIKAQKKAQSSTDSGSETPGTSESAT
PESDKKYSIGLAIGTNSVGWAVITDEYKVPSKKFKVLGNTDRHSI
KKNLIGALLFDSGETAEATRLKRTARRRYTRRKNRICYLQEIFSN
EMAKVDDSFFHRLEESFLVEEDKKHERHPIFGNIVDEVAYHEKYP
TIYHLRKKLVDSTDKADLRLIYLALAHMIKFRGHFLIEGDLNPDN
SDVDKLFIQLVQTYNQLFEENPINASGVDAKAILSARLSKSRRLE
NLIAQLPGEKKNGLFGNLIALSLGLTPNFKSNFDLAEDAKLQLSK
DTYDDDLDNLLAQIGDQYADLFLAAKNLSDAILLSDILRVNTEIT
KAPLSASMIKRYDEHHQDLTLLKALVRQQLPEKYKEIFFDQSKNG
YAGYIDGGASQEEFYKFIKPILEKMDGTEELLVKLNREDLLRKQR
TFDNGSIPHQIHLGELHAILRRQEDFYPFLKDNREKIEKILTFRI
PYYVGPLARGNSRFAWMTRKSEETITPWNFEEVVDKGASAQSFIE
RMTNFDKNLPNEKVLPKHSLLYEYFTVYNELTKVKYVTEGMRKPA
FLSGEQKKAIVDLLFKTNRKVTVKQLKEDYFKKIECFDSVEISGV
EDRFNASLGTYHDLLKIIKDKDFLDNEENEDILEDIVLTLTLFED
REMIEERLKTYAHLFDDKVMKQLKRRRYTGWGRLSRKLINGIRDK
QSGKTILDFLKSDGFANRNFMQLIHDDSLTFKEDIQKAQVSGQGD
SLHEHIANLAGSPAIKKGILQTVKVVDELVKVMGRHKPENIVIEM
ARENQTTQKGQKNSRERMKRIEEGIKELGSQILKEHPVENTQLQN
EKLYLYYLQNGRDMYVDQELDINRLSDYDVDHIVPQSFLKDDSID
NKVLTRSDKNRGKSDNVPSEEVVKKMKNYWRQLLNAKLITQRKFD
NLTKAERGGLSELDKAGFIKRQLVETRQITKHVAQILDSRMNTKY
DENDKLIREVKVITLKSKLVSDFRKDFQFYKVREINNYHHAHDAY
LNAVVGTALIKKYPKLESEFVYGDYKVYDVRKMIAKSEQEIGKAT
AKYFFYSNIMNFFKTEITLANGEIRKRPLIETNGETGEIVWDKGR
DFATVRKVLSMPQVNIVKKTEVQTGGFSKESILPKRNSDKLIARK
KDWDPKKYGGFDSPTVAYSVLVVAKVEKGKSKKLKSVKELLGITI
MERSSFEKNPIDFLEAKGYKEVKKDLIIKLPKYSLFELENGRKRM
LASAGELQKGNELALPSKYVNFLYLASHYEKLKGSPEDNEQKQLF
VEQHKHYLDEIIEQISEFSKRVILADANLDKVLSAYNKHRDKPIR
EQAENIIHLFTLTNLGAPAAFKYFDTTIDRKRYTSTKEVLDATLI
HQSITGLYETRIDLSQLGGDSGGSPKKKRKV
ecTadA(D108V)-XTEN-nCas9-NLS
(mammalian construct, active on DNA,
A to G editing):
(SEQ ID NO: 177)
MSEVEFSHEYWMRHALTLAKRAWDEREVPVGAVLVHNNRVIGEGW
NRPIGRHDPTAHAEIMALRQGGLVMQNYRLIDATLYVTLEPCVMC
AGAMIHSRIGRVVFGARVAKTGAAGSLMDVLHHPGMNHRVEITEG
ILADECAALLSDFFRMRRQEIKAQKKAQSSTDSGSETPGTSESAT
PESDKKYSIGLAIGTNSVGWAVITDEYKVPSKKFKVLGNTDRHSI
KKNLIGALLFDSGETAEATRLKRTARRRYTRRKNRICYLQEIFSN
EMAKVDDSFFHRLEESFLVEEDKKHERHPIFGNIVDEVAYHEKYP
TIYHLRKKLVDSTDKADLRLIYLALAHMIKFRGHFLIEGDLNPDN
SDVDKLFIQLVQTYNQLFEENPINASGVDAKAILSARLSKSRRLE
NLIAQLPGEKKNGLFGNLIALSLGLTPNFKSNFDLAEDAKLQLSK
DTYDDDLDNLLAQIGDQYADLFLAAKNLSDAILLSDILRVNTEIT
KAPLSASMIKRYDEHHQDLTLLKALVRQQLPEKYKEIFFDQSKNG
YAGYIDGGASQEEFYKFIKPILEKMDGTEELLVKLNREDLLRKQR
TFDNGSIPHQIHLGELHAILRRQEDFYPFLKDNREKIEKILTFRI
PYYVGPLARGNSRFAWMTRKSEETITPWNFEEVVDKGASAQSFIE
RMTNFDKNLPNEKVLPKHSLLYEYFTVYNELTKVKYVTEGMRKPA
FLSGEQKKAIVDLLFKTNRKVTVKQLKEDYFKKIECFDSVEISGV
EDRFNASLGTYHDLLKIIKDKDFLDNEENEDILEDIVLTLTLFED
REMIEERLKTYAHLFDDKVMKQLKRRRYTGWGRLSRKLINGIRDK
QSGKTILDFLKSDGFANRNFMQLIHDDSLTFKEDIQKAQVSGQGD
SLHEHIANLAGSPAIKKGILQTVKVVDELVKVMGRHKPENIVIEM
ARENQTTQKGQKNSRERMKRIEEGIKELGSQILKEHPVENTQLQN
EKLYLYYLQNGRDMYVDQELDINRLSDYDVDHIVPQSFLKDDSID
NKVLTRSDKNRGKSDNVPSEEVVKKMKNYWRQLLNAKLITQRKFD
NLTKAERGGLSELDKAGFIKRQLVETRQITKHVAQILDSRMNTKY
DENDKLIREVKVITLKSKLVSDFRKDFQFYKVREINNYHHAHDAY
LNAVVGTALIKKYPKLESEFVYGDYKVYDVRKMIAKSEQEIGKAT
AKYFFYSNIMNFFKTEITLANGEIRKRPLIETNGETGEIVWDKGR
DFATVRKVLSMPQVNIVKKTEVQTGGFSKESILPKRNSDKLIARK
KDWDPKKYGGFDSPTVAYSVLVVAKVEKGKSKKLKSVKELLGITI
MERSSFEKNPIDFLEAKGYKEVKKDLIIKLPKYSLFELENGRKRM
LASAGELQKGNELALPSKYVNFLYLASHYEKLKGSPEDNEQKQLF
VEQHKHYLDEIIEQISEFSKRVILADANLDKVLSAYNKHRDKPIR
EQAENIIHLFTLTNLGAPAAFKYFDTTIDRKRYTSTKEVLDATLI
HQSITGLYETRIDLSQLGGDSGGSPKKKRKV
ecTadA(H8Y_D108N_N127S)-XTEN-dCas9
(variant resulting from first round of
evolution in bacteria):
(SEQ ID NO: 178)
MSEVEFSYEYWMRHALTLAKRAWDEREVPVGAVLVHNNRVIGEGW
NRPIGRHDPTAHAEIMALRQGGLVMQNYRLIDATLYVTLEPCVMC
AGAMIHSRIGRVVFGARNAKTGAAGSLMDVLHHPGMSHRVEITEG
ILADECAALLSDFFRMRRQEIKAQKKAQSSTDSGSETPGTSESAT
PESDKKYSIGLAIGTNSVGWAVITDEYKVPSKKFKVLGNTDRHSI
KKNLIGALLFDSGETAEATRLKRTARRRYTRRKNRICYLQEIFSN
EMAKVDDSFFHRLEESFLVEEDKKHERHPIFGNIVDEVAYHEKYP
TIYHLRKKLVDSTDKADLRLIYLALAHMIKFRGHFLIEGDLNPDN
SDVDKLFIQLVQTYNQLFEENPINASGVDAKAILSARLSKSRRLE
NLIAQLPGEKKNGLFGNLIALSLGLTPNFKSNFDLAEDAKLQLSK
DTYDDDLDNLLAQIGDQYADLFLAAKNLSDAILLSDILRVNTEIT
KAPLSASMIKRYDEHHQDLTLLKALVRQQLPEKYKEIFFDQSKNG
YAGYIDGGASQEEFYKFIKPILEKMDGTEELLVKLNREDLLRKQR
TFDNGSIPHQIHLGELHAILRRQEDFYPFLKDNREKIEKILTFRI
PYYVGPLARGNSRFAWMTRKSEETITPWNFEEVVDKGASAQSFIE
RMTNFDKNLPNEKVLPKHSLLYEYFTVYNELTKVKYVTEGMRKPA
FLSGEQKKAIVDLLFKTNRKVTVKQLKEDYFKKIECFDSVEISGV
EDRFNASLGTYHDLLKIIKDKDFLDNEENEDILEDIVLTLTLFED
REMIEERLKTYAHLFDDKVMKQLKRRRYTGWGRLSRKLINGIRDK
QSGKTILDFLKSDGFANRNFMQLIHDDSLTFKEDIQKAQVSGQGD
SLHEHIANLAGSPAIKKGILQTVKVVDELVKVMGRHKPENIVIEM
ARENQTTQKGQKNSRERMKRIEEGIKELGSQILKEHPVENTQLQN
EKLYLYYLQNGRDMYVDQELDINRLSDYDVDAIVPQSFLKDDSID
NKVLTRSDKNRGKSDNVPSEEVVKKMKNYWRQLLNAKLITQRKFD
NLTKAERGGLSELDKAGFIKRQLVETRQITKHVAQILDSRMNTKY
DENDKLIREVKVITLKSKLVSDFRKDFQFYKVREINNYHHAHDAY
LNAVVGTALIKKYPKLESEFVYGDYKVYDVRKMIAKSEQEIGKAT
AKYFFYSNIMNFFKTEITLANGEIRKRPLIETNGETGEIVWDKGR
DFATVRKVLSMPQVNIVKKTEVQTGGFSKESILPKRNSDKLIARK
KDWDPKKYGGFDSPTVAYSVLVVAKVEKGKSKKLKSVKELLGITI
MERSSFEKNPIDFLEAKGYKEVKKDLIIKLPKYSLFELENGRKRM
LASAGELQKGNELALPSKYVNFLYLASHYEKLKGSPEDNEQKQLF
VEQHKHYLDEIIEQISEFSKRVILADANLDKVLSAYNKHRDKPIR
EQAENIIHLFTLTNLGAPAAFKYFDTTIDRKRYTSTKEVLDATLI
HQSITGLYETRIDLSQLGGD
(H8Y_D108N_N127S_E155X)-XTEN-dCas9;
X = D, G or V
(Enriched variants from second round of
evolution (in bacteria) ecTadA):
(SEQ ID NO: 179)
MSEVEFS [symbol] EYWMRHALTLAKRAWDEREVPVGAVLVHNNRVIGE
GWNRPIGRHDPTAHAEIMALRQGGLVMQNYRLIDATLYVTLEP
CVMCAGAMIHSRIGRVVFGAR [symbol] AKTGAAGSLMDVLHHPGM [symbol]
HRVEITEGILADECAALLSDFFRMRRQ [symbol] IKAQKKAQSSTD
SGSETPGTSESATPESDKKYSIGLAIGTNSVGWAVITDEYKVPSK
KFKVLGNTDRHSIKKNLIGALLFDSGETAEATRLKRTARRRYTRR
KNRICYLQEIFSNEMAKVDDSFFHRLEESFLVEEDKKHERHPIFG
NIVDEVAYHEKYPTIYHLRKKLVDSTDKADLRLIYLALAHMIKFR
GHFLIEGDLNPDNSDVDKLFIQLVQTYNQLFEENPINASGVDAKA
ILSARLSKSRRLENLIAQLPGEKKNGLFGNLIALSLGLTPNFKSN
FDLAEDAKLQLSKDTYDDDLDNLLAQIGDQYADLFLAAKNLSDAI
LLSDILRVNTEITKAPLSASMIKRYDEHHQDLTLLKALVRQQLPE
KYKEIFFDQSKNGYAGYIDGGASQEEFYKFIKPILEKMDGTEELL
VKLNREDLLRKQRTFDNGSIPHQIHLGELHAILRRQEDFYPFLKD
NREKIEKILTFRIPYYVGPLARGNSRFAWMTRKSEETITPWNFEE
VVDKGASAQSFIERMTNFDKNLPNEKVLPKHSLLYEYFTVYNELT
KVKYVTEGMRKPAFLSGEQKKAIVDLLFKTNRKVTVKQLKEDYFK
KIECFDSVEISGVEDRFNASLGTYHDLLKIIKDKDFLDNEENEDI
LEDIVLTLTLFEDREMIEERLKTYAHLFDDKVMKQLKRRRYTGWG
RLSRKLINGIRDKQSGKTILDFLKSDGFANRNFMQLIHDDSLTFK
EDIQKAQVSGQGDSLHEHIANLAGSPAIKKGILQTVKVVDELVKV
MGRHKPENIVIEMARENQTTQKGQKNSRERMKRIEEGIKELGSQI
LKEHPVENTQLQNEKLYLYYLQNGRDMYVDQELDINRLSDYDVDA
IVPQSFLKDDSIDNKVLTRSDKNRGKSDNVPSEEVVKKMKNYWRQ
LLNAKLITQRKFDNLTKAERGGLSELDKAGFIKRQLVETRQITKH
VAQILDSRMNTKYDENDKLIREVKVITLKSKLVSDFRKDFQFYKV
REINNYHHAHDAYLNAVVGTALIKKYPKLESEFVYGDYKVYDVRK
MIAKSEQEIGKATAKYFFYSNIMNFFKTEITLANGEIRKRPLIET
NGETGEIVWDKGRDFATVRKVLSMPQVNIVKKTEVQTGGFSKESI
LPKRNSDKLIARKKDWDPKKYGGFDSPTVAYSVLVVAKVEKGKSK
KLKSVKELLGITIMERSSFEKNPIDFLEAKGYKEVKKDLIKLPKY
SLFELENGRKRMLASAGELQKGNELALPSKYVNFLYLASHYEKLK
GSPEDNEQKQLFVEQHKHYLDEIIEQISEFSKRVILADANLDKVL
SAYNKHRDKPIREQAENIIHLFTLTNLGAPAAFKYFDTTIDRKRY
TSTKEVLDATLIHQSITGLYETRIDLSQLGGD
ABE7.7
ecTadA_{(wild-type)}-(SGGS)₂₋XTEN-(SGGS)₂-
ecTadA_{(W23L}_H36L_P48A_R51L_L84F_A106V_D108N_
_{H123Y}_S146C_D147Y_R152P_ E155V_1156F KIS7N)-(SGGS)₂-
XTEN-(SGGS)₂_nCas9_SGGS_NLS
(SEQ ID NO: 180)
MSEVEFSHEYWMRHALTLAKRAWDEREVPVGAVLVHNNRVIGEGW
NRPIGRHDPTAHAEIMALRQGGLVMQNYRLIDATLYVTLEPCVMC
AGAMIHSRIGRVVFGARDAKTGAAGSLMDVLHHPGMNHRVEITEG
ILADECAALLSDFFRMRRQEIKAQKKAQSSTDSGGSSGGSSGSET
PGTSESATPESSGGSSGGSSEVEFSHEYWMRHALTLAKRALDERE
VPVGAVLVLNNRVIGEGWNRAIGLHDPTAHAEIMALRQGGLVMQN
YRLIDATLYVTFEPCVMCAGAMIHSRIGRVVFGVRNAKTGAAGSL
MDVLHYPGMNHRVEITEGILADECAALLCYFFRMPRQVFNAQKKA
QSSTDSGGSSGGSSGSETPGTSESATPESSGGSSGGSD
KKYSIGLAIGTNSVGWAVITDEYKVPSKKFKVLGNTDRHSIKKNL
IGALLFDSGETAEATRLKRTARRRYTRRKNRICYLQEIFSNEMAK
VDDSFFHRLEESFLVEEDKKHERHPIFGNIVDEVAYHEKYPTIYH
LRKKLVDSTDKADLRLIYLALAHMIKFRGHFLIEGDLNPDNSDVD
KLFIQLVQTYNQLFEENPINASGVDAKAILSARLSKSRRLENLIA
QLPGEKKNGLFGNLIALSLGLTPNFKSNFDLAEDAKLQLSKDTYD
DDLDNLLAQIGDQYADLFLAAKNLSDAILLSDILRVNTEITKAPL
SASMIKRYDEHHQDLTLLKALVRQQLPEKYKEIFFDQSKNGYAGY
IDGGASQEEFYKFIKPILEKMDGTEELLVKLNREDLLRKQRTFDN
GSIPHQIHLGELHAILRRQEDFYPFLKDNREKIEKILTFRIPYYV
GPLARGNSRFAWMTRKSEETITPWNFEEVVDKGASAQSFIERMTN
FDKNLPNEKVLPKHSLLYEYFTVYNELTKVKYVTEGMRKPAFLSG
EQKKAIVDLLFKTNRKVTVKQLKEDYFKKIECFDSVEISGVEDRF
NASLGTYHDLLKIIKDKDFLDNEENEDILEDIVLTLTLFEDREMI
EERLKTYAHLFDDKVMKQLKRRRYTGWGRLSRKLINGIRDKQSGK
TILDFLKSDGFANRNFMQLIHDDSLTFKEDIQKAQVSGQGDSLHE
HIANLAGSPAIKKGILQTVKVVDELVKVMGRHKPENIVIEMAREN
QTTQKGQKNSRERMKRIEEGIKELGSQILKEHPVENTQLQNEKLY
LYYLQNGRDMYVDQELDINRLSDYDVDHIVPQSFLKDDSIDNKVL
TRSDKNRGKSDNVPSEEVVKKMKNYWRQLLNAKLITQRKFDNLTK
AERGGLSELDKAGFIKRQLVETRQITKHVAQILDSRMNTKYDEND
KLIREVKVITLKSKLVSDFRKDFQFYKVREINNYHHAHDAYLNAV
VGTALIKKYPKLESEFVYGDYKVYDVRKMIAKSEQEIGKATAKYF
FYSNIMNFFKTEITLANGEIRKRPLIETNGETGEIVWDKGRDFAT
VRKVLSMPQVNIVKKTEVQTGGFSKESILPKRNSDKLIARKKDWD
PKKYGGFDSPTVAYSVLVVAKVEKGKSKKLKSVKELLGITIMERS
SFEKNPIDFLEAKGYKEVKKDLIIKLPKYSLFELENGRKRMLASA
GELQKGNELALPSKYVNFLYLASHYEKLKGSPEDNEQKQLFVEQH
KHYLDEHIEQISEFSKRVILADANLDKVLSAYNKHRDKPIREQAE
NIIHLFTLTNLGAPAAFKYFDTTIDRKRYTSTKEVLDATLIHQSI
TGLYETRIDLSQLGGDSGGSPKKKRKV
pNMG-624
ecTadA_{(wild-type)}-32 a.a. linker-
ecTadA_{(W23R}_H36L_P48A_R51L_L84F_A106V_D108N_H123Y_
_{S146C}_Di47Y_Ri52P_Ei55v_ii56F_Ki57N)-24 a.a.
linker nCas9 SGGS _NLS
(SEQ ID NO: 181)
MSEVEFSHEYWMRHALTLAKRAWDEREVPVGAVLVHNNRVIGEGW
NRPIGRHDPTAHAEIMALRQGGLVMQNYRLIDATLYVTLEPCVMC
AGAMIHSRIGRVVFGARDAKTGAAGSLMDVLHHPGMNHRVEITEG
ILADECAALLSDFFRMRRQEIKAQKKAQSSTDSGGSSGGSSGSET
PGTSESATPESSGGSSGGSSEVEFSHEYWMRHALTLAKRARDERE
VPVGAVLVLNNRVIGEGWNRAIGLHDPTAHAEIMALRQGGLVMQN
YRLIDATLYVTFEPCVMCAGAMIHSRIGRVVFGVRNAKTGAAGSL
MDVLHYPGMNHRVEITEGILADECAALLCYFFRMPRQVFNAQKKA
QSSTDSGGSSGGSSGSETPGTSESATPESDKKYSIGLAIGTNSVG
WAVITDEYKVPSKKFKVLGNTDRHSIKKNLIGALLFDSGETAEAT
RLKRTARRRYTRRKNRICYLQEIFSNEMAKVDDSFFHRLEESFLV
EEDKKHERHPIFGNIVDEVAYHEKYPTIYHLRKKLVDSTDKADLR
LIYLALAHMIKFRGHFLIEGDLNPDNSDVDKLFIQLVQTYNQLFE
ENPINASGVDAKAILSARLSKSRRLENLIAQLPGEKKNGLFGNLI
ALSLGLTPNFKSNFDLAEDAKLQLSKDTYDDDLDNLLAQIGDQYA
DLFLAAKNLSDAILLSDILRVNTEITKAPLSASMIKRYDEHHQDL
TLLKALVRQQLPEKYKEIFFDQSKNGYAGYIDGGASQEEFYKFIK
PILEKMDGTEELLVKLNREDLLRKQRTFDNGSIPHQIHLGELHAI
LRRQEDFYPFLKDNREKIEKILTFRIPYYVGPLARGNSRFAWMTR
KSEETITPWNFEEVVDKGASAQSFIERMTNFDKNLPNEKVLPKHS
LLYEYFTVYNELTKVKYVTEGMRKPAFLSGEQKKAIVDLLFKTNR
KVTVKQLKEDYFKKIECFDSVEISGVEDRFNASLGTYHDLLKIIK
DKDFLDNEENEDILEDIVLTLTLFEDREMIEERLKTYAHLFDDKV
MKQLKRRRYTGWGRLSRKLINGIRDKQSGKTILDFLKSDGFANRN
FMQLIHDDSLTFKEDIQKAQVSGQGDSLHEHIANLAGSPAIKKGI
LQTVKVVDELVKVMGRHKPENIVIEMARENQTTQKGQKNSRERMK
RIEEGIKELGSQILKEHPVENTQLQNEKLYLYYLQNGRDMYVDQE
LDINRLSDYDVDHIVPQSFLKDDSIDNKVLTRSDKNRGKSDNVPS
EEVVKKMKNYWRQLLNAKLITQRKFDNLTKAERGGLSELDKAGFI
KRQLVETRQITKHVAQILDSRMNTKYDENDKLIREVKVITLKSKL
VSDFRKDFQFYKVREINNYHHAHDAYLNAVVGTALIKKYPKLESE
FVYGDYKVYDVRKMIAKSEQEIGKATAKYFFYSNIMNFFKTEITL
ANGEIRKRPLIETNGETGEIVWDKGRDFATVRKVLSMPQVNIVKK
TEVQTGGFSKESILPKRNSDKLIARKKDWDPKKYGGFDSPTVAYS
VLVVAKVEKGKSKKLKSVKELLGITIMERSSFEKNPIDFLEAKGY
KEVKKDLIIKLPKYSLFELENGRKRMLASAGELQKGNELALPSKY
VNFLYLASHYEKLKGSPEDNEQKQLFVEQHKHYLDEIIEQISEFS
KRVILADANLDKVLSAYNKHRDKPIREQAENIIHLFTLTNLGAPA
AFKYFDTTIDRKRYTSTKEVLDATLIHQSITGLYETRIDLSQLGG
DSGGSPKKKRKV
ABE3.2
ecTadA_{(wild-type)}-(SGGS)₂-XTEN-(SGGS)₂-
ecTadA_{(L84F}_A106V_D108N_H123Y_D147Y_E155V_1156F)-
(SGGS)₂-XTEN-(SGGS)₂_nCas9_SGGS_NLS
(SEQ ID NO: 182)
MSEVEFSHEYWMRHALTLAKRAWDEREVPVGAVLVHNNRVIGEGW
NRPIGRHDPTAHAEIMALRQGGLVMQNYRLIDATLYVTLEPCVMC
AGAMIHSRIGRVVFGARDAKTGAAGSLMDVLHHPGMNHRVEITEG
ILADECAALLSDFFRMRRQEIKAQKKAQSSTDSGGSSGGSSGSET
PGTSESATPESSGGSSGGSSEVEFSHEYWMRHALTLAKRAWDERE
VPVGAVLVHNNRVIGEGWNRPIGRHDPTAHAEIMALRQGGLVMQN
YRLIDATLYVTFEPCVMCAGAMIHSRIGRVVFGVRNAKTGAAGSL
MDVLHYPGMNHRVEITEGILADECAALLSYFFRMRRQVFKAQKKA
QSSTDSGGSSGGSSGSETPGTSESATPESSGGSSGGSDKKYSIGL
AIGTNSVGWAVITDEYKVPSKKFKVLGNTDRHSIKKNLIGALLFD
SGETAEATRLKRTARRRYTRRKNRICYLQEIFSNEMAKVDDSFFH
RLEESFLVEEDKKHERHPIFGNIVDEVAYHEKYPTIYHLRKKLVD
STDKADLRLIYLALAHMIKFRGHFLIEGDLNPDNSDVDKLFIQLV
QTYNQLFEENPINASGVDAKAILSARLSKSRRLENLIAQLPGEKK
NGLFGNLIALSLGLTPNFKSNFDLAEDAKLQLSKDTYDDDLDNLL
AQIGDQYADLFLAAKNLSDAILLSDILRVNTEITKAPLSASMIKR
YDEHHQDLTLLKALVRQQLPEKYKEIFFDQSKNGYAGYIDGGASQ
EEFYKFIKPILEKMDGTEELLVKLNREDLLRKQRTFDNGSIPHQI
HLGELHAILRRQEDFYPFLKDNREKIEKILTFRIPYYVGPLARGN
SRFAWMTRKSEETITPWNFEEVVDKGASAQSFIERMTNFDKNLPN
EKVLPKHSLLYEYFTVYNELTKVKYVTEGMRKPAFLSGEQKKAIV
DLLFKTNRKVTVKQLKEDYFKKIECFDSVEISGVEDRFNASLGTY
HDLLKIIKDKDFLDNEENEDILEDIVLTLTLFEDREMIEERLKTY
AHLFDDKVMKQLKRRRYTGWGRLSRKLINGIRDKQSGKTILDFLK
SDGFANRNFMQLIHDDSLTFKEDIQKAQVSGQGDSLHEHIANLAG
SPAIKKGILQTVKVVDELVKVMGRHKPENIVIEMARENQTTQKGQ
KNSRERMKRIEEGIKELGSQILKEHPVENTQLQNEKLYLYYLQNG
RDMYVDQELDINRLSDYDVDHIVPQSFLKDDSIDNKVLTRSDKNR
GKSDNVPSEEVVKKMKNYWRQLLNAKLITQRKFDNLTKAERGGLS
ELDKAGFIKRQLVETRQITKHVAQILDSRMNTKYDENDKLIREVK
VITLKSKLVSDFRKDFQFYKVREINNYHHAHDAYLNAVVGTALIK
KYPKLESEFVYGDYKVYDVRKMIAKSEQEIGKATAKYFFYSNIMN
FFKTEITLANGEIRKRPLIETNGETGEIVWDKGRDFATVRKVLSM
PQVNIVKKTEVQTGGFSKESILPKRNSDKLIARKKDWDPKKYGGF
DSPTVAYSVLVVAKVEKGKSKKLKSVKELLGITIMERSSFEKNPI
DFLEAKGYKEVKKDLIIKLPKYSLFELENGRKRMLASAGELQKGN
ELALPSKYVNFLYLASHYEKLKGSPEDNEQKQLFVEQHKHYLDEH
IEQISEFSKRVILADANLDKVLSAYNKHRDKPIREQAENIIHLFT
LTNLGAPAAFKYFDTTIDRKRYTSTKEVLDATLIHQSITGLYETR
IDLSQLGGDSGGSPKKKRKV
ABE5.3
ecTadA_{(wild-type)}-(SGGS)₂-XTEN-(SGGS)₂-
ecTadA_{(H36L}_R51L_L84F_A106V_D108N_H123Y_S146C_
_{D147Y}_E155V_I156F_K157N)-(SGGS)₂-XTEN-(SGGS)_
nCas9_SGGS_NLS
(SEQ ID NO: 183)
MSEVEFSHEYWMRHALTLAKRAWDEREVPVGAVLVHNNRVIGEGW
NRPIGRHDPTAHAEIMALRQGGLVMQNYRLIDATLYVTLEPCVMC
AGAMIHSRIGRVVFGARDAKTGAAGSLMDVLHHPGMNHRVEITEG
ILADECAALLSDFFRMRRQEIKAQKKAQSSTDSGGSSGGSSGSET
PGTSESATPESSGGSSGGSSEVEFSHEYWMRHALTLAKRAWDERE
VPVGAVLVLNNRVIGEGWNRPIGLHDPTAHAEIMALRQGGLVMQN
YRLIDATLYVTFEPCVMCAGAMIHSRIGRVVFGVRNAKTGAAGSL
MDVLHYPGMNHRVEITEGILADECAALLCYFFRMRRQVFNAQKKA
QSSTDSGGSSGGSSGSETPGTSESATPESSGGSSGGSDKKYSIGL
AIGTNSVGWAVITDEYKVPSKKFKVLGNTDRHSIKKNLIGALLFD
SGETAEATRLKRTARRRYTRRKNRICYLQEIFSNEMAKVDDSFFH
RLEESFLVEEDKKHERHPIFGNIVDEVAYHEKYPTIYHLRKKLVD
STDKADLRLIYLALAHMIKFRGHFLIEGDLNPDNSDVDKLFIQLV
QTYNQLFEENPINASGVDAKAILSARLSKSRRLENLIAQLPGEKK
NGLFGNLIALSLGLTPNFKSNFDLAEDAKLQLSKDTYDDDLDNLL
AQIGDQYADLFLAAKNLSDAILLSDILRVNTEITKAPLSASMIKR
YDEHHQDLTLLKALVRQQLPEKYKEIFFDQSKNGYAGYIDGGASQ
EEFYKFIKPILEKMDGTEELLVKLNREDLLRKQRTFDNGSIPHQI
HLGELHAILRRQEDFYPFLKDNREKIEKILTFRIPYYVGPLARGN
SRFAWMTRKSEETITPWNFEEVVDKGASAQSFIERMTNFDKNLPN
EKVLPKHSLLYEYFTVYNELTKVKYVTEGMRKPAFLSGEQKKAIV
DLLFKTNRKVTVKQLKEDYFKKIECFDSVEISGVEDRFNASLGTY
HDLLKIIKDKDFLDNEENEDILEDIVLTLTLFEDREMIEERLKTY
AHLFDDKVMKQLKRRRYTGWGRLSRKLINGIRDKQSGKTILDFLK
SDGFANRNFMQLIHDDSLTFKEDIQKAQVSGQGDSLHEHIANLAG
SPAIKKGILQTVKVVDELVKVMGRHKPENIVIEMARENQTTQKGQ
KNSRERMKRIEEGIKELGSQILKEHPVENTQLQNEKLYLYYLQNG
RDMYVDQELDINRLSDYDVDHIVPQSFLKDDSIDNKVLTRSDKNR
GKSDNVPSEEVVKKMKNYWRQLLNAKLITQRKFDNLTKAERGGLS
ELDKAGFIKRQLVETRQITKHVAQILDSRMNTKYDENDKLIREVK
VITLKSKLVSDFRKDFQFYKVREINNYHHAHDAYLNAVVGTALIK
KYPKLESEFVYGDYKVYDVRKMIAKSEQEIGKATAKYFFYSNIMN
FFKTEITLANGEIRKRPLIETNGETGEIVWDKGRDFATVRKVLSM
PQVNIVKKTEVQTGGFSKESILPKRNSDKLIARKKDWDPKKYGGF
DSPTVAYSVLVVAKVEKGKSKKLKSVKELLGITIMERSSFEKNPI
DFLEAKGYKEVKKDLIIKLPKYSLFELENGRKRMLASAGELQKGN
ELALPSKYVNFLYLASHYEKLKGSPEDNEQKQLFVEQHKHYLDEH
IEQISEFSKRVILADANLDKVLSAYNKHRDKPIREQAENIIHLFT
LTNLGAPAAFKYFDTTIDRKRYTSTKEVLDATLIHQSITGLYETR
IDLSQLGGD
SGGSPKKKRKV
pNMG-558
ecTadA(wild-type)- 32 a.a. linker-
ecTadA_{(H36L}_R51L_L84F_A106V_D108N_H123Y_S146C_D147Y_ E155V_1156F
_K157N)⁻ 24 a.a. linker nCas9 SGGS NLS
(SEQ ID NO: 184)
MSEVEFSHEYWMRHALTLAKRAWDEREVPVGAVLVHNNRVIGEGW
NRPIGRHDPTAHAEIMALRQGGLVMQNYRLIDATLYVTLEPCVMC
AGAMIHSRIGRVVFGARDAKTGAAGSLMDVLHHPGMNHRVEITEG
ILADECAALLSDFFRMRRQEIKAQKKAQSSTDSGGSSGGSSGSET
PGTSESATPESSGGSSGGSSEVEFSHEYWMRHALTLAKRAWDERE
VPVGAVLVLNNRVIGEGWNRPIGLHDPTAHAEIMALRQGGLVMQN
YRLIDATLYVTFEPCVMCAGAMIHSRIGRVVFGVRNAKTGAAGSL
MDVLHYPGMNHRVEITEGILADECAALLCYFFRMRRQVFNAQKKA
QSSTDSGGSSGGSSGSETPGTSESATPESDKKYSIGLAIGTNSVG
WAVITDEYKVPSKKFKVLGNTDRHSIKKNLIGALLFDSGETAEAT
RLKRTARRRYTRRKNRICYLQEIFSNEMAKVDDSFFHRLEESFLV
EEDKKHERHPIFGNIVDEVAYHEKYPTIYHLRKKLVDSTDKADLR
LIYLALAHMIKFRGHFLIEGDLNPDNSDVDKLFIQLVQTYNQLFE
ENPINASGVDAKAILSARLSKSRRLENLIAQLPGEKKNGLFGNLI
ALSLGLTPNFKSNFDLAEDAKLQLSKDTYDDDLDNLLAQIGDQYA
DLFLAAKNLSDAILLSDILRVNTEITKAPLSASMIKRYDEHHQDL
TLLKALVRQQLPEKYKEIFFDQSKNGYAGYIDGGASQEEFYKFIK
PILEKMDGTEELLVKLNREDLLRKQRTFDNGSIPHQIHLGELHAI
LRRQEDFYPFLKDNREKIEKILTFRIPYYVGPLARGNSRFAWMTR
KSEETITPWNFEEVVDKGASAQSFIERMTNFDKNLPNEKVLPKHS
LLYEYFTVYNELTKVKYVTEGMRKPAFLSGEQKKAIVDLLFKTNR
KVTVKQLKEDYFKKIECFDSVEISGVEDRFNASLGTYHDLLKIIK
DKDFLDNEENEDILEDIVLTLTLFEDREMIEERLKTYAHLFDDKV
MKQLKRRRYTGWGRLSRKLINGIRDKQSGKTILDFLKSDGFANRN
FMQLIHDDSLTFKEDIQKAQVSGQGDSLHEHIANLAGSPAIKKGI
LQTVKVVDELVKVMGRHKPENIVIEMARENQTTQKGQKNSRERMK
RIEEGIKELGSQILKEHPVENTQLQNEKLYLYYLQNGRDMYVDQE
LDINRLSDYDVDHIVPQSFLKDDSIDNKVLTRSDKNRGKSDNVPS
EEVVKKMKNYWRQLLNAKLITQRKFDNLTKAERGGLSELDKAGFI
KRQLVETRQITKHVAQILDSRMNTKYDENDKLIREVKVITLKSKL
VSDFRKDFQFYKVREINNYHHAHDAYLNAVVGTALIKKYPKLESE
FVYGDYKVYDVRKMIAKSEQEIGKATAKYFFYSNIMNFFKTEITL
ANGEIRKRPLIETNGETGEIVWDKGRDFATVRKVLSMPQVNIVKK
TEVQTGGFSKESILPKRNSDKLIARKKDWDPKKYGGFDSPTVAYS
VLVVAKVEKGKSKKLKSVKELLGITIMERSSFEKNPIDFLEAKGY
KEVKKDLIIKLPKYSLFELENGRKRMLASAGELQKGNELALPSKY
VNFLYLASHYEKLKGSPEDNEQKQLFVEQHKHYLDEIIEQISEFS
KRVILADANLDKVLSAYNKHRDKPIREQAENIIHLFTLTNLGAPA
AFKYFDTTIDRKRYTSTKEVLDATLIHQSITGLYETRIDLSQLGG
DSGGSPKKKRKV
pNMG-576
ecTadA(wild-type)-(SGGS)₂-XTEN-(SGGS)₂-
ecTadA_{(H36L}_P48S_R51L_L84F_A106V_D108N_
_{H123Y}_S146C_D147Y_E155V_I156F_K157N)-
(SGGS)₂-XTEN-(SGGS)_nCas9_GGSNLS
(SEQ ID NO: 185)
MSEVEFSHEYWMRHALTLAKRAWDEREVPVGAVLVHNNRVIGEGW
NRPIGRHDPTAHAEIMALRQGGLVMQNYRLIDATLYVTLEPCVMC
AGAMIHSRIGRVVFGARDAKTGAAGSLMDVLHHPGMNHRVEITEG
ILADECAALLSDFFRMRRQEIKAQKKAQSSTDSGGSSGGSSGSET
PGTSESATPESSGGSSGGSSEVEFSHEYWMRHALTLAKRAWDERE
VPVGAVLVLNNRVIGEGWNRSIGLHDPTAHAEIMALRQGGLVMQN
YRLIDATLYVTFEPCVMCAGAMIHSRIGRVVFGVRNAKTGAAGSL
MDVLHYPGMNHRVEITEGILADECAALLCYFFRMRRQVFNAQKKA
QSSTDSGGSSGGSSGSETPGTSESATPESSGGSSGGSDKKYSIGL
AIGTNSVGWAVITDEYKVPSKKFKVLGNTDRHSIKKNLIGALLFD
SGETAEATRLKRTARRRYTRRKNRICYLQEIFSNEMAKVDDSFFH
RLEESFLVEEDKKHERHPIFGNIVDEVAYHEKYPTIYHLRKKLVD
STDKADLRLIYLALAHMIKFRGHFLIEGDLNPDNSDVDKLFIQLV
QTYNQLFEENPINASGVDAKAILSARLSKSRRLENLIAQLPGEKK
NGLFGNLIALSLGLTPNFKSNFDLAEDAKLQLSKDTYDDDLDNLL
AQIGDQYADLFLAAKNLSDAILLSDILRVNTEITKAPLSASMIKR
YDEHHQDLTLLKALVRQQLPEKYKEIFFDQSKNGYAGYIDGGASQ
EEFYKFIKPILEKMDGTEELLVKLNREDLLRKQRTFDNGSIPHQI
HLGELHAILRRQEDFYPFLKDNREKIEKILTFRIPYYVGPLARGN
SRFAWMTRKSEETITPWNFEEVVDKGASAQSFIERMTNFDKNLPN
EKVLPKHSLLYEYFTVYNELTKVKYVTEGMRKPAFLSGEQKKAIV
DLLFKTNRKVTVKQLKEDYFKKIECFDSVEISGVEDRFNASLGTY
HDLLKIIKDKDFLDNEENEDILEDIVLTLTLFEDREMIEERLKTY
AHLFDDKVMKQLKRRRYTGWGRLSRKLINGIRDKQSGKTILDFLK
SDGFANRNFMQLIHDDSLTFKEDIQKAQVSGQGDSLHEHIANLAG
SPAIKKGILQTVKVVDELVKVMGRHKPENIVIEMARENQTTQKGQ
KNSRERMKRIEEGIKELGSQILKEHPVENTQLQNEKLYLYYLQNG
RDMYVDQELDINRLSDYDVDHIVPQSFLKDDSIDNKVLTRSDKNR
GKSDNVPSEEVVKKMKNYWRQLLNAKLITQRKFDNLTKAERGGLS
ELDKAGFIKRQLVETRQITKHVAQILDSRMNTKYDENDKLIREVK
VITLKSKLVSDFRKDFQFYKVREINNYHHAHDAYLNAVVGTALIK
KYPKLESEFVYGDYKVYDVRKMIAKSEQEIGKATAKYFFYSNIMN
FFKTEITLANGEIRKRPLIETNGETGEIVWDKGRDFATVRKVLSM
PQVNIVKKTEVQTGGFSKESILPKRNSDKLIARKKDWDPKKYGGF
DSPTVAYSVLVVAKVEKGKSKKLKSVKELLGITIMERSSFEKNPI
DFLEAKGYKEVKKDLIIKLPKYSLFELENGRKRMLASAGELQKGN
ELALPSKYVNFLYLASHYEKLKGSPEDNEQKQLFVEQHKHYLDEH
IEQISEFSKRVILADANLDKVLSAYNKHRDKPIREQAENIIHLFT
LTNLGAPAAFKYFDTTIDRKRYTSTKEVLDATLIHQSITGLYETR
IDLSQLGGD
SGGSPKKKRKV
pNMG-577
ecTadA(wild-type)-(SGGS)₂-XTEN-(SGGS)₂-
ecTadA_{(H36L}_P48S_R51L_L84F_A106V_D108N_H123Y_
_{A142N}_S146C_D147Y_E155V_I156F_K157N)-(SGGS)-XTEN-
(SGGS)₂_nCas9GGS_NLS
(SEQ ID NO: 186)
MSEVEFSHEYWMRHALTLAKRAWDEREVPVGAVLVHNNRVIGEGW
NRPIGRHDPTAHAEIMALRQGGLVMQNYRLIDATLYVTLEPCVMC
AGAMIHSRIGRVVFGARDAKTGAAGSLMDVLHHPGMNHRVEITEG
ILADECAALLSDFFRMRRQEIKAQKKAQSSTDSGGSSGGSSGSET
PGTSESATPESSGGSSGGSSEVEFSHEYWMRHALTLAKRAWDERE
VPVGAVLVLNNRVIGEGWNRSIGLHDPTAHAEIMALRQGGLVMQN
YRLIDATLYVTFEPCVMCAGAMIHSRIGRVVFGVRNAKTGAAGSL
MDVLHYPGMNHRVEITEGILADECNALLCYFFRMRRQVFNAQKKA
QSSTDSGGSSGGSSGSETPGTSESATPESSGGSSGGSDKKYSIGL
AIGTNSVGWAVITDEYKVPSKKFKVLGNTDRHSIKKNLIGALLFD
SGETAEATRLKRTARRRYTRRKNRICYLQEIFSNEMAKVDDSFFH
RLEESFLVEEDKKHERHPIFGNIVDEVAYHEKYPTIYHLRKKLVD
STDKADLRLIYLALAHMIKFRGHFLIEGDLNPDNSDVDKLFIQLV
QTYNQLFEENPINASGVDAKAILSARLSKSRRLENLIAQLPGEKK
NGLFGNLIALSLGLTPNFKSNFDLAEDAKLQLSKDTYDDDLDNLL
AQIGDQYADLFLAAKNLSDAILLSDILRVNTEITKAPLSASMIKR
YDEHHQDLTLLKALVRQQLPEKYKEIFFDQSKNGYAGYIDGGASQ
EEFYKFIKPILEKMDGTEELLVKLNREDLLRKQRTFDNGSIPHQI
HLGELHAILRRQEDFYPFLKDNREKIEKILTFRIPYYVGPLARGN
SRFAWMTRKSEETITPWNFEEVVDKGASAQSFIERMTNFDKNLPN
EKVLPKHSLLYEYFTVYNELTKVKYVTEGMRKPAFLSGEQKKAIV
DLLFKTNRKVTVKQLKEDYFKKIECFDSVEISGVEDRFNASLGTY
HDLLKIIKDKDFLDNEENEDILEDIVLTLTLFEDREMIEERLKTY
AHLFDDKVMKQLKRRRYTGWGRLSRKLINGIRDKQSGKTILDFLK
SDGFANRNFMQLIHDDSLTFKEDIQKAQVSGQGDSLHEHIANLAG
SPAIKKGILQTVKVVDELVKVMGRHKPENIVIEMARENQTTQKGQ
KNSRERMKRIEEGIKELGSQILKEHPVENTQLQNEKLYLYYLQNG
RDMYVDQELDINRLSDYDVDHIVPQSFLKDDSIDNKVLTRSDKNR
GKSDNVPSEEVVKKMKNYWRQLLNAKLITQRKFDNLTKAERGGLS
ELDKAGFIKRQLVETRQITKHVAQILDSRMNTKYDENDKLIREVK
VITLKSKLVSDFRKDFQFYKVREINNYHHAHDAYLNAVVGTALIK
KYPKLESEFVYGDYKVYDVRKMIAKSEQEIGKATAKYFFYSNIMN
FFKTEITLANGEIRKRPLIETNGETGEIVWDKGRDFATVRKVLSM
PQVNIVKKTEVQTGGFSKESILPKRNSDKLIARKKDWDPKKYGGF
DSPTVAYSVLVVAKVEKGKSKKLKSVKELLGITIMERSSFEKNPI
DFLEAKGYKEVKKDLIIKLPKYSLFELENGRKRMLASAGELQKGN
ELALPSKYVNFLYLASHYEKLKGSPEDNEQKQLFVEQHKHYLDEH
IEQISEFSKRVILADANLDKVLSAYNKHRDKPIREQAENIIHLFT
LTNLGAPAAFKYFDTTIDRKRYTSTKEVLDATLIHQSITGLYETR
IDLSQLGGD
SGGSPKKKRKV
pNMG-586
ecTadA(wild-type)-(SGGS)₂-XTEN-(SGGS)₂-
ecTadA_{(H36L}_P48A_R51L_L84F_A106V_D108N_
_{H123Y}_S146C_D147Y_E155V_I156F_K157N)⁻
(SGGS)₂-XTEN-(SGGS)₂_nCas9_GGS_NLS
(SEQ ID NO: 187)
MSEVEFSHEYWMRHALTLAKRAWDEREVPVGAVLVHNNRVIGEGW
NRPIGRHDPTAHAEIMALRQGGLVMQNYRLIDATLYVTLEPCVMC
AGAMIHSRIGRVVFGARDAKTGAAGSLMDVLHHPGMNHRVEITEG
ILADECAALLSDFFRMRRQEIKAQKKAQSSTDSGGSSGGSSGSET
PGTSESATPESSGGSSGGSSEVEFSHEYWMRHALTLAKRAWDERE
VPVGAVLVLNNRVIGEGWNRAIGLHDPTAHAEIMALRQGGLVMQN
YRLIDATLYVTFEPCVMCAGAMIHSRIGRVVFGVRNAKTGAAGSL
MDVLHYPGMNHRVEITEGILADECAALLCYFFRMRRQVFNAQKKA
QSSTDSGGSSGGSSGSETPGTSESATPESSGGSSGGSDKKYSIGL
AIGTNSVGWAVITDEYKVPSKKFKVLGNTDRHSIKKNLIGALLFD
SGETAEATRLKRTARRRYTRRKNRICYLQEIFSNEMAKVDDSFFH
RLEESFLVEEDKKHERHPIFGNIVDEVAYHEKYPTIYHLRKKLVD
STDKADLRLIYLALAHMIKFRGHFLIEGDLNPDNSDVDKLFIQLV
QTYNQLFEENPINASGVDAKAILSARLSKSRRLENLIAQLPGEKK
NGLFGNLIALSLGLTPNFKSNFDLAEDAKLQLSKDTYDDDLDNLL
AQIGDQYADLFLAAKNLSDAILLSDILRVNTEITKAPLSASMIKR
YDEHHQDLTLLKALVRQQLPEKYKEIFFDQSKNGYAGYIDGGASQ
EEFYKFIKPILEKMDGTEELLVKLNREDLLRKQRTFDNGSIPHQI
HLGELHAILRRQEDFYPFLKDNREKIEKILTFRIPYYVGPLARGN
SRFAWMTRKSEETITPWNFEEVVDKGASAQSFIERMTNFDKNLPN
EKVLPKHSLLYEYFTVYNELTKVKYVTEGMRKPAFLSGEQKKAIV
DLLFKTNRKVTVKQLKEDYFKKIECFDSVEISGVEDRFNASLGTY
HDLLKIIKDKDFLDNEENEDILEDIVLTLTLFEDREMIEERLKTY
AHLFDDKVMKQLKRRRYTGWGRLSRKLINGIRDKQSGKTILDFLK
SDGFANRNFMQLIHDDSLTFKEDIQKAQVSGQGDSLHEHIANLAG
SPAIKKGILQTVKVVDELVKVMGRHKPENIVIEMARENQTTQKGQ
KNSRERMKRIEEGIKELGSQILKEHPVENTQLQNEKLYLYYLQNG
RDMYVDQELDINRLSDYDVDHIVPQSFLKDDSIDNKVLTRSDKNR
GKSDNVPSEEVVKKMKNYWRQLLNAKLITQRKFDNLTKAERGGLS
ELDKAGFIKRQLVETRQITKHVAQILDSRMNTKYDENDKLIREVK
VITLKSKLVSDFRKDFQFYKVREINNYHHAHDAYLNAVVGTALIK
KYPKLESEFVYGDYKVYDVRKMIAKSEQEIGKATAKYFFYSNIMN
FFKTEITLANGEIRKRPLIETNGETGEIVWDKGRDFATVRKVLSM
PQVNIVKKTEVQTGGFSKESILPKRNSDKLIARKKDWDPKKYGGF
DSPTVAYSVLVVAKVEKGKSKKLKSVKELLGITIMERSSFEKNPI
DFLEAKGYKEVKKDLIIKLPKYSLFELENGRKRMLASAGELQKGN
ELALPSKYVNFLYLASHYEKLKGSPEDNEQKQLFVEQHKHYLDEH
IEQISEFSKRVILADANLDKVLSAYNKHRDKPIREQAENIIHLFT
LTNLGAPAAFKYFDTTIDRKRYTSTKEVLDATLIHQSITGLYETR
IDLSQLGGD
SGGSPKKKRKV
ABE7.2
ecTadA(wild-type)-(SGGS)₂-XTEN-(SGGS)₂-
ecTadA_{(H36L}_P48A_R51L_L84F_A106V_D108N_
_{H123Y}_A142N_S146C_D147Y_E155V_I156F K157N)-
(SGGS)₂-XTEN-(SGGS)₂_nCas9_GGS_NLS
(SEQ ID NO: 188)
MSEVEFSHEYWMRHALTLAKRAWDEREVPVGAVLVHNNRVIGEGW
NRPIGRHDPTAHAEIMALRQGGLVMQNYRLIDATLYVTLEPCVMC
AGAMIHSRIGRVVFGARDAKTGAAGSLMDVLHHPGMNHRVEITEG
ILADECAALLSDFFRMRRQEIKAQKKAQSSTDSGGSSGGSSGSET
PGTSESATPESSGGSSGGSSEVEFSHEYWMRHALTLAKRAWDERE
VPVGAVLVLNNRVIGEGWNRAIGLHDPTAHAEIMALRQGGLVMQN
YRLIDATLYVTFEPCVMCAGAMIHSRIGRVVFGVRNAKTGAAGSL
MDVLHYPGMNHRVEITEGILADECNALLCYFFRMRRQVFNAQKKA
QSSTDSGGSSGGSSGSETPGTSESATPESSGGSSGGSDKKYSIGL
AIGTNSVGWAVITDEYKVPSKKFKVLGNTDRHSIKKNLIGALLFD
SGETAEATRLKRTARRRYTRRKNRICYLQEIFSNEMAKVDDSFFH
RLEESFLVEEDKKHERHPIFGNIVDEVAYHEKYPTIYHLRKKLVD
STDKADLRLIYLALAHMIKFRGHFLIEGDLNPDNSDVDKLFIQLV
QTYNQLFEENPINASGVDAKAILSARLSKSRRLENLIAQLPGEKK
NGLFGNLIALSLGLTPNFKSNFDLAEDAKLQLSKDTYDDDLDNLL
AQIGDQYADLFLAAKNLSDAILLSDILRVNTEITKAPLSASMIKR
YDEHHQDLTLLKALVRQQLPEKYKEIFFDQSKNGYAGYIDGGASQ
EEFYKFIKPILEKMDGTEELLVKLNREDLLRKQRTFDNGSIPHQI
HLGELHAILRRQEDFYPFLKDNREKIEKILTFRIPYYVGPLARGN
SRFAWMTRKSEETITPWNFEEVVDKGASAQSFIERMTNFDKNLPN
EKVLPKHSLLYEYFTVYNELTKVKYVTEGMRKPAFLSGEQKKAIV
DLLFKTNRKVTVKQLKEDYFKKIECFDSVEISGVEDRFNASLGTY
HDLLKIIKDKDFLDNEENEDILEDIVLTLTLFEDREMIEERLKTY
AHLFDDKVMKQLKRRRYTGWGRLSRKLINGIRDKQSGKTILDFLK
SDGFANRNFMQLIHDDSLTFKEDIQKAQVSGQGDSLHEHIANLAG
SPAIKKGILQTVKVVDELVKVMGRHKPENIVIEMARENQTTQKGQ
KNSRERMKRIEEGIKELGSQILKEHPVENTQLQNEKLYLYYLQNG
RDMYVDQELDINRLSDYDVDHIVPQSFLKDDSIDNKVLTRSDKNR
GKSDNVPSEEVVKKMKNYWRQLLNAKLITQRKFDNLTKAERGGLS
ELDKAGFIKRQLVETRQITKHVAQILDSRMNTKYDENDKLIREVK
VITLKSKLVSDFRKDFQFYKVREINNYHHAHDAYLNAVVGTALIK
KYPKLESEFVYGDYKVYDVRKMIAKSEQEIGKATAKYFFYSNIMN
FFKTEITLANGEIRKRPLIETNGETGEIVWDKGRDFATVRKVLM
PQVNIVKKTEVQTGGFSKESILPKRNSDKLIARKKDWDPKKYGGF
DSPTVAYSVLVVAKVEKGKSKKLKSVKELLGITIMERSSFEKNPI
DFLEAKGYKEVKKDLIIKLPKYSLFELENGRKRMLASAGELQKGN
ELALPSKYVNFLYLASHYEKLKGSPEDNEQKQLFVEQHKHYLDEI
IEQISEFSKRVILADANLDKVLSAYNKHRDKPIREQAENIIHLFT
LTNLGAPAAFKYFDTTIDRKRYTSTKEVLDATLIHQSITGLYETR
IDLSQLGGD
SGGSPKKKRKV
pNMG-620
ecTadA_{(wild-type)}-(SGGS)₂-XTEN-(SGGS)₂-
ecTadA_{(W23R}_H36L_P48A_R51L_L84F_A106V_D108N_
_{H123Y}_S146C_D147Y_R152P_E155V_I156F K157N)-
(SGGS)-XTEN-(SGGS)₂_nCas9GGS_NLS
(SEQ ID NO: 189)
MSEVEFSHEYWMRHALTLAKRAWDEREVPVGAVLVHNNRVIGEGW
NRPIGRHDPTAHAEIMALRQGGLVMQNYRLIDATLYVTLEPCVMC
AGAMIHSRIGRVVFGARDAKTGAAGSLMDVLHHPGMNHRVEITEG
ILADECAALLSDFFRMRRQEIKAQKKAQSSTDSGGSSGGSSGSET
PGTSESATPESSGGSSGGSSEVEFSHEYWMRHALTLAKRARDERE
VPVGAVLVLNNRVIGEGWNRAIGLHDPTAHAEIMALRQGGLVMQN
YRLIDATLYVTFEPCVMCAGAMIHSRIGRVVFGVRNAKTGAAGSL
MDVLHYPGMNHRVEITEGILADECAALLCYFFRMPRQVFNAQKKA
QSSTDSGGSSGGSSGSETPGTSESATPESSGGSSGGSDKKYSIGL
AIGTNSVGWAVITDEYKVPSKKFKVLGNTDRHSIKKNLIGALLFD
SGETAEATRLKRTARRRYTRRKNRICYLQEIFSNEMAKVDDSFFH
RLEESFLVEEDKKHERHPIFGNIVDEVAYHEKYPTIYHLRKKLVD
STDKADLRLIYLALAHMIKFRGHFLIEGDLNPDNSDVDKLFIQLV
QTYNQLFEENPINASGVDAKAILSARLSKSRRLENLIAQLPGEKK
NGLFGNLIALSLGLTPNFKSNFDLAEDAKLQLSKDTYDDDLDNLL
AQIGDQYADLFLAAKNLSDAILLSDILRVNTEITKAPLSASMIKR
YDEHHQDLTLLKALVRQQLPEKYKEIFFDQSKNGYAGYIDGGASQ
EEFYKFIKPILEKMDGTEELLVKLNREDLLRKQRTFDNGSIPHQI
HLGELHAILRRQEDFYPFLKDNREKIEKILTFRIPYYVGPLARGN
SRFAWMTRKSEETITPWNFEEVVDKGASAQSFIERMTNFDKNLPN
EKVLPKHSLLYEYFTVYNELTKVKYVTEGMRKPAFLSGEQKKAIV
DLLFKTNRKVTVKQLKEDYFKKIECFDSVEISGVEDRFNASLGTY
HDLLKIIKDKDFLDNEENEDILEDIVLTLTLFEDREMIEERLKTY
AHLFDDKVMKQLKRRRYTGWGRLSRKLINGIRDKQSGKTILDFLK
SDGFANRNFMQLIHDDSLTFKEDIQKAQVSGQGDSLHEHIANLAG
SPAIKKGILQTVKVVDELVKVMGRHKPENIVIEMARENQTTQKGQ
KNSRERMKRIEEGIKELGSQILKEHPVENTQLQNEKLYLYYLQNG
RDMYVDQELDINRLSDYDVDHIVPQSFLKDDSIDNKVLTRSDKNR
GKSDNVPSEEVVKKMKNYWRQLLNAKLITQRKFDNLTKAERGGLS
ELDKAGFIKRQLVETRQITKHVAQILDSRMNTKYDENDKLIREVK
VITLKSKLVSDFRKDFQFYKVREINNYHHAHDAYLNAVVGTALIK
KYPKLESEFVYGDYKVYDVRKMIAKSEQEIGKATAKYFFYSNIMN
FFKTEITLANGEIRKRPLIETNGETGEIVWDKGRDFATVRKVLSM
PQVNIVKKTEVQTGGFSKESILPKRNSDKLIARKKDWDPKKYGGF
DSPTVAYSVLVVAKVEKGKSKKLKSVKELLGITIMERSSFEKNPI
DFLEAKGYKEVKKDLIIKLPKYSLFELENGRKRMLASAGELQKGN
ELALPSKYVNFLYLASHYEKLKGSPEDNEQKQLFVEQHKHYLDEI
IEQISEFSKRVILADANLDKVLSAYNKHRDKPIREQAENIIHLFT
LTNLGAPAAFKYFDTTIDRKRYTSTKEVLDATLIHQSITGLYETR
IDLSQLGGD
SGGSPKKKRKV
pNMG-617
ecTadA_{(wild-type)}-(SGGS)₂-XTEN-(SGGS)₂-
ecTadA_{(W23L}_H36L_P48A_R51L_L84F_A1Q6V_D1Q8N_
_{H123Y}_A142A_S146C_D147Y_E155V_I156F K157N)-
(SGGS)-XTEN-(SGGS)₂_nCas9GGS_NLS
(SEQ ID NO: 190)
MSEVEFSHEYWMRHALTLAKRAWDEREVPVGAVLVHNNRVIGEGW
NRPIGRHDPTAHAEIMALRQGGLVMQNYRLIDATLYVTLEPCVMC
AGAMIHSRIGRVVFGARDAKTGAAGSLMDVLHHPGMNHRVEITEG
ILADECAALLSDFFRMRRQEIKAQKKAQSSTDSGGSSGGSSGSET
PGTSESATPESSGGSSGGSSEVEFSHEYWMRHALTLAKRALDERE
VPVGAVLVLNNRVIGEGWNRAIGLHDPTAHAEIMALRQGGLVMQN
YRLIDATLYVTFEPCVMCAGAMIHSRIGRVVFGVRNAKTGAAGSL
MDVLHYPGMNHRVEITEGILADECNALLCYFFRMRRQVFNAQKKA
QSSTDSGGSSGGSSGSETPGTSESATPESSGGSSGGSDKKYSIGL
AIGTNSVGWAVITDEYKVPSKKFKVLGNTDRHSIKKNLIGALLFD
SGETAEATRLKRTARRRYTRRKNRICYLQEIFSNEMAKVDDSFFH
RLEESFLVEEDKKHERHPIFGNIVDEVAYHEKYPTIYHLRKKLVD
STDKADLRLIYLALAHMIKFRGHFLIEGDLNPDNSDVDKLFIQLV
QTYNQLFEENPINASGVDAKAILSARLSKSRRLENLIAQLPGEKK
NGLFGNLIALSLGLTPNFKSNFDLAEDAKLQLSKDTYDDDLDNLL
AQIGDQYADLFLAAKNLSDAILLSDILRVNTEITKAPLSASMIKR
YDEHHQDLTLLKALVRQQLPEKYKEIFFDQSKNGYAGYIDGGASQ
EEFYKFIKPILEKMDGTEELLVKLNREDLLRKQRTFDNGSIPHQI
HLGELHAILRRQEDFYPFLKDNREKIEKILTFRIPYYVGPLARGN
SRFAWMTRKSEETITPWNFEEVVDKGASAQSFIERMTNFDKNLPN
EKVLPKHSLLYEYFTVYNELTKVKYVTEGMRKPAFLSGEQKKAIV
DLLFKTNRKVTVKQLKEDYFKKIECFDSVEISGVEDRFNASLGTY
HDLLKIIKDKDFLDNEENEDILEDIVLTLTLFEDREMIEERLKTY
AHLFDDKVMKQLKRRRYTGWGRLSRKLINGIRDKQSGKTILDFLK
SDGFANRNFMQLIHDDSLTFKEDIQKAQVSGQGDSLHEHIANLAG
SPAIKKGILQTVKVVDELVKVMGRHKPENIVIEMARENQTTQKGQ
KNSRERMKRIEEGIKELGSQILKEHPVENTQLQNEKLYLYYLQNG
RDMYVDQELDINRLSDYDVDHIVPQSFLKDDSIDNKVLTRSDKNR
GKSDNVPSEEVVKKMKNYWRQLLNAKLITQRKFDNLTKAERGGLS
ELDKAGFIKRQLVETRQITKHVAQILDSRMNTKYDENDKLIREVK
VITLKSKLVSDFRKDFQFYKVREINNYHHAHDAYLNAVVGTALIK
KYPKLESEFVYGDYKVYDVRKMIAKSEQEIGKATAKYFFYSNIMN
FFKTEITLANGEIRKRPLIETNGETGEIVWDKGRDFATVRKVLSM
PQVNTVKKTEVQTGGFSKESILPKRNSDKLIARKKDWDPKKYGGF
DSPTVAYSVLVVAKVEKGKSKKLKSVKELLGITIMERSSFEKNPI
DFLEAKGYKEVKKDLIIKLPKYSLFELENGRKRMLASAGELQKGN
ELALPSKYVNFLYLASHYEKLKGSPEDNEQKQLFVEQHKHYLDEH
IEQISEFSKRVILADANLDKVLSAYNKHRDKPIREQAENIIHLFT
LTNLGAPAAFKYFDTTIDRKRYTSTKEVLDATLIHQSITGLYETR
IDLSQLGGD
SGGSPKKKRKV
pNMG-618
ecTadA_{(wild-type)}-(SGGS)₂-XTEN-(SGGS)₂-
ecTadA_{(W23L}_H36L_P48A_R51L_L84F_A106V_D108N_
(SEQ ID NO: 191)
_{H123Y}_A142A_S146C_D147Y R152P E155V
_{I156F K157N)}-(SGGS)₂-XTEN-(SGGS)₂ nCas9_GGS_NLS
MSEVEFSHEYWMRHALTLAKRAWDEREVPVGAVLVHNNRVIGEGW
NRPIGRHDPTAHAEIMALRQGGLVMQNYRLIDATLYVTLEPCVMC
AGAMIHSRIGRVVFGARDAKTGAAGSLMDVLHHPGMNHRVEITEG
ILADECAALLSDFFRMRRQEIKAQKKAQSSTDSGGSSGGSSGSET
PGTSESATPESSGGSSGGSSEVEFSHEYWMRHALTLAKRALDERE
VPVGAVLVLNNRVIGEGWNRAIGLHDPTAHAEIMALRQGGLVMQN
YRLIDATLYVTFEPCVMCAGAMIHSRIGRVVFGVRNAKTGAAGSL
MDVLHYPGMNHRVEITEGILADECNALLCYFFRMPRQVFNAQKKA
QSSTDSGGSSGGSSGSETPGTSESATPESSGGSSGGSDKKYSIGL
AIGTNSVGWAVITDEYKVPSKKFKVLGNTDRHSIKKNLIGALLFD
SGETAEATRLKRTARRRYTRRKNRICYLQEIFSNEMAKVDDSFFH
RLEESFLVEEDKKHERHPIFGNIVDEVAYHEKYPTIYHLRKKLVD
STDKADLRLIYLALAHMIKFRGHFLIEGDLNPDNSDVDKLFIQLV
QTYNQLFEENPINASGVDAKAILSARLSKSRRLENLIAQLPGEKK
NGLFGNLIALSLGLTPNFKSNFDLAEDAKLQLSKDTYDDDLDNLL
AQIGDQYADLFLAAKNLSDAILLSDILRVNTEITKAPLSASMIKR
YDEHHQDLTLLKALVRQQLPEKYKEIFFDQSKNGYAGYIDGGASQ
EEFYKFIKPILEKMDGTEELLVKLNREDLLRKQRTFDNGSIPHQI
HLGELHAILRRQEDFYPFLKDNREKIEKILTFRIPYYVGPLARGN
SRFAWMTRKSEETITPWNFEEVVDKGASAQSFIERMTNFDKNLPN
EKVLPKHSLLYEYFTVYNELTKVKYVTEGMRKPAFLSGEQKKAIV
DLLFKTNRKVTVKQLKEDYFKKIECFDSVEISGVEDRFNASLGTY
HDLLKIIKDKDFLDNEENEDILEDIVLTLTLFEDREMIEERLKTY
AHLFDDKVMKQLKRRRYTGWGRLSRKLINGIRDKQSGKTILDFLK
SDGFANRNFMQLIHDDSLTFKEDIQKAQVSGQGDSLHEHIANLAG
SPAIKKGILQTVKVVDELVKVMGRHKPENIVIEMARENQTTQKGQ
KNSRERMKRIEEGIKELGSQILKEHPVENTQLQNEKLYLYYLQNG
RDMYVDQELDINRLSDYDVDHIVPQSFLKDDSIDNKVLTRSDKNR
GKSDNVPSEEVVKKMKNYWRQLLNAKLITQRKFDNLTKAERGGLS
ELDKAGFIKRQLVETRQITKHVAQILDSRMNTKYDENDKLIREVK
VITLKSKLVSDFRKDFQFYKVREINNYHHAHDAYLNAVVGTALIK
KYPKLESEFVYGDYKVYDVRKMIAKSEQEIGKATAKYFFYSNIMN
FFKTEITLANGEIRKRPLIETNGETGEIVWDKGRDFATVRKVLSM
PQVNIVKKTEVQTGGFSKESILPKRNSDKLIARKKDWDKKYGGF
DSPTVAYSVLVVAKVEKGKSKKLKSVKELLGITIMERSSFEKNPI
DFLEAKGYKEVKKDLIIKLPKYSLFELENGRKRMLASAGELQKGN
ELALPSKYVNFLYLASHYEKLKGSPEDNEQKQLFVEQHKHYLDEH
IEQISEFSKRVILADANLDKVLSAYNKHRDKPIREQAENIIHLFT
LTNLGAPAAFKYFDTTIDRKRYTSTKEVLDATLIHQSITGLYETR
IDLSQLGGD
SGGSPKKKRKV
pNMG-620
ecTadA _{(wild-type)}-(SGGS)₂-XTEN-(SGGS)₂-
ecTadA_{(W23R}_H36L_P48A_R51L_L84F_
_{A106V}_D108N_
_{H123Y}_S146C_D147Y_R152P_E155V_I156F K157N)-
(SGGS)-XTEN-(SGGS)₂_nCas9GGS_NLS
(SEQ ID NO: 192)
MSEVEFSHEYWMRHALTLAKRAWDEREVPVGAVLVHNNRVIGEGW
NRPIGRHDPTAHAEIMALRQGGLVMQNYRLIDATLYVTLEPCVMC
AGAMIHSRIGRVVFGARDAKTGAAGSLMDVLHHPGMNHRVEITEG
ILADECAALLSDFFRMRRQEIKAQKKAQSSTDSGGSSGGSSGSET
PGTSESATPESSGGSSGGSSEVEFSHEYWMRHALTLAKRARDERE
VPVGAVLVLNNRVIGEGWNRAIGLHDPTAHAEIMALRQGGLVMQN
YRLIDATLYVTFEPCVMCAGAMIHSRIGRVVFGVRNAKTGAAGSL
MDVLHYPGMNHRVEITEGILADECAALLCYFFRMPRQVFNAQKKA
QSSTDSGGSSGGSSGSETPGTSESATPESSGGSSGGSDKKYSIGL
AIGTNSVGWAVITDEYKVPSKKFKVLGNTDRHSIKKNLIGALLFD
SGETAEATRLKRTARRRYTRRKNRICYLQEIFSNEMAKVDDSFFH
RLEESFLVEEDKKHERHPIFGNIVDEVAYHEKYPTIYHLRKKLVD
STDKADLRLIYLALAHMIKFRGHFLIEGDLNPDNSDVDKLFIQLV
QTYNQLFEENPINASGVDAKAILSARLSKSRRLENLIAQLPGEKK
NGLFGNLIALSLGLTPNFKSNFDLAEDAKLQLSKDTYDDDLDNLL
AQIGDQYADLFLAAKNLSDAILLSDILRVNTEITKAPLSASMIKR
YDEHHQDLTLLKALVRQQLPEKYKEIFFDQSKNGYAGYIDGGASQ
EEFYKFIKPILEKMDGTEELLVKLNREDLLRKQRTFDNGSIPHQI
HLGELHAILRRQEDFYPFLKDNREKIEKILTFRIPYYVGPLARGN
SRFAWMTRKSEETITPWNFEEVVDKGASAQSFIERMTNFDKNLPN
EKVLPKHSLLYEYFTVYNELTKVKYVTEGMRKPAFLSGEQKKAIV
DLLFKTNRKVTVKQLKEDYFKKIECFDSVEISGVEDRFNASLGTY
HDLLKIIKDKDFLDNEENEDILEDIVLTLTLFEDREMIEERLKTY
AHLFDDKVMKQLKRRRYTGWGRLSRKLINGIRDKQSGKTILDFLK
SDGFANRNFMQLIHDDSLTFKEDIQKAQVSGQGDSLHEHIANLAG
SPAIKKGILQTVKVVDELVKVMGRHKPENTVIEMARENQTTQKGQ
KNSRERMKRIEEGIKELGSQILKEHPVENTQLQNEKLYLYYLQNG
RDMYVDQELDINRLSDYDVDHIVPQSFLKDDSIDNKVLTRSDKNR
GKSDNVPSEEVVKKMKNYWRQLLNAKLITQRKFDNLTKAERGGLS
ELDKAGFIKRQLVETRQITKHVAQILDSRMNTKYDENDKLIREVK
VITLKSKLVSDFRKDFQFYKVREINNYHHAHDAYLNAVVGTALIK
KYPKLESEFVYGDYKVYDVRKMIAKSEQEIGKATAKYFFYSNIMN
FFKTEITLANGEIRKRPLIETNGETGEIVWDKGRDFATVRKVLSM
PQVNIVKKTEVQTGGFSKESILPKRNSDKLIARKKDWDPKKYGGF
DSPTVAYSVLVVAKVEKGKSKKLKSVKELLGITIMERSSFEKNPI
DFLEAKGYKEVKKDLIIKLPKYSLFELENGRKRMLASAGELQKGN
ELALPSKYVNFLYLASHYEKLKGSPEDNEQKQLFVEQHKHYLDEH
IEQISEFSKRVILADANLDKVLSAYNKHRDKPIREQAENIIHLFT
LTNLGAPAAFKYFDTTIDRKRYTSTKEVLDATLIHQSITGLYETR
IDLSQLGGD
SGGSPKKKRKV
pNMG-621
ecTadA_{(wild-type)}- 32 a.a. linker-
ecTadA_{(H36L}_P48A_R51L_L84F_
_{A106V}_D108N_H123Y_
_{S146C}_D147Y_R152P_E155V_H56F_K157N)-
24 a.a. linker nCas9 GGS NLS
(SEQ ID NO:193)
MSEVEFSHEYWMRHALTLAKRAWDEREVPVGAVLVHNNRVIGEGW
NRPIGRHDPTAHAEIMALRQGGLVMQNYRLIDATLYVTLEPCVMC
AGAMIHSRIGRVVFGARDAKTGAAGSLMDVLHHPGMNHRVEITEG
ILADECAALLSDFFRMRRQEIKAQKKAQSSTDSGGSSGGSSGSET
PGTSESATPESSGGSSGGSSEVEFSHEYWMRHALTLAKRAWDERE
VPVGAVLVLNNRVIGEGWNRAIGLHDPTAHAEIMALRQGGLVMQN
YRLIDATLYVTFEPCVMCAGAMIHSRIGRVVFGVRNAKTGAAGSL
MDVLHYPGMNHRVEITEGILADECAALLCYFFRMPRQVFNAQKKA
QSSTDSGGSSGGSSGSETPGTSESATPESDKKYSIGLAIGTNSVG
WAVITDEYKVPSKKFKVLGNTDRHSIKKNLIGALLFDSGETAEAT
RLKRTARRRYTRRKNRICYLQEIFSNEMAKVDDSFFHRLEESFLV
EEDKKHERHPIFGNIVDEVAYHEKYPTIYHLRKKLVDSTDKADLR
LIYLALAHMIKFRGHFLIEGDLNPDNSDVDKLFIQLVQTYNQLFE
ENPINASGVDAKAILSARLSKSRRLENLIAQLPGEKKNGLFGNLI
ALSLGLTPNFKSNFDLAEDAKLQLSKDTYDDDLDNLLAQIGDQYA
DLFLAAKNLSDAILLSDILRVNTEITKAPLSASMIKRYDEHHQDL
TLLKALVRQQLPEKYKEIFFDQSKNGYAGYIDGGASQEEFYKFIK
PILEKMDGTEELLVKLNREDLLRKQRTFDNGSIPHQIHLGELHAI
LRRQEDFYPFLKDNREKIEKILTFRIPYYVGPLARGNSRFAWMTR
KSEETITPWNFEEVVDKGASAQSFIERMTNFDKNLPNEKVLPKHS
LLYEYFTVYNELTKVKYVTEGMRKPAFLSGEQKKAIVDLLFKTNR
KVTVKQLKEDYFKKIECFDSVEISGVEDRFNASLGTYHDLLKIIK
DKDFLDNEENEDILEDIVLTLTLFEDREMIEERLKTYAHLFDDKV
MKQLKRRRYTGWGRLSRKLINGIRDKQSGKTILDFLKSDGFANRN
FMQLIHDDSLTFKEDIQKAQVSGQGDSLHEHIANLAGSPAIKKGI
LQTVKVVDELVKVMGRHKPENIVIEMARENQTTQKGQKNSRERMK
RIEEGIKELGSQILKEHPVENTQLQNEKLYLYYLQNGRDMYVDQE
LDINRLSDYDVDHIVPQSFLKDDSIDNKVLTRSDKNRGKSDNVPS
EEVVKKMKNYWRQLLNAKLITQRKFDNLTKAERGGLSELDKAGFI
KRQLVETRQITKHVAQILDSRMNTKYDENDKLIREVKVITLKSKL
VSDFRKDFQFYKVREINNYHHAHDAYLNAVVGTALIKKYPKLESE
FVYGDYKVYDVRKMIAKSEQEIGKATAKYFFYSNIMNFFKTEITL
ANGEIRKRPLIETNGETGEIVWDKGRDFATVRKVLSMPQVNIVKK
TEVQTGGFSKESILPKRNSDKLIARKKDWDPKKYGGFDSPTVAYS
VLVVAKVEKGKSKKLKSVKELLGITIMERSSFEKNPIDFLEAKGY
KEVKKDLIIKLPKYSLFELENGRKRMLASAGELQKGNELALPSKY
VNFLYLASHYEKLKGSPEDNEQKQLFVEQHKHYLDEIIEQISEFS
KRVILADANLDKVLSAYNKHRDKPIREQAENIIHLFTLTNLGAPA
AFKYFDTTIDRKRYTSTKEVLDATLIHQSITGLYETRIDLSQLGG
DSGGSPKKKRKV
pNMG-622
ecTadA_{(wild-type)}- 32 a.a. linker-
ecTadA_{(H36L}_P48A_R51L_L84F_A106V_
_{D108N}_H123Y_A142N_
_{S146C}_D147Y_R152P_E155V_H56F_K157N)-
24 a.a. linker nCas9_GGS_NLS
(SEQ ID NO: 194)
MSEVEFSHEYWMRHALTLAKRAWDEREVPVGAVLVHNNRVIGEGW
NRPIGRHDPTAHAEIMALRQGGLVMQNYRLIDATLYVTLEPCVMC
AGAMIHSRIGRVVFGARDAKTGAAGSLMDVLHHPGMNHRVEITEG
ILADECAALLSDFFRMRRQEIKAQKKAQSSTDSGGSSGGSSGSET
PGTSESATPESSGGSSGGSSEVEFSHEYWMRHALTLAKRAWDERE
VPVGAVLVLNNRVIGEGWNRAIGLHDPTAHAEIMALRQGGLVMQN
YRLIDATLYVTFEPCVMCAGAMIHSRIGRVVFGVRNAKTGAAGSL
MDVLHYPGMNHRVEITEGILADECNALLCYFFRMPRQVFNAQKKA
QSSTDSGGSSGGSSGSETPGTSESATPESDKKYSIGLAIGTNSVG
WAVITDEYKVPSKKFKVLGNTDRHSIKKNLIGALLFDSGETAEAT
RLKRTARRRYTRRKNRICYLQEIFSNEMAKVDDSFFHRLEESFLV
EEDKKHERHPIFGNIVDEVAYHEKYPTIYHLRKKLVDSTDKADLR
LIYLALAHMIKFRGHFLIEGDLNPDNSDVDKLFIQLVQTYNQLFE
ENPINASGVDAKAILSARLSKSRRLENLIAQLPGEKKNGLFGNLI
ALSLGLTPNFKSNFDLAEDAKLQLSKDTYDDDLDNLLAQIGDQYA
DLFLAAKNLSDAILLSDILRVNTEITKAPLSASMIKRYDEHHQDL
TLLKALVRQQLPEKYKEIFFDQSKNGYAGYIDGGASQEEFYKFIK
PILEKMDGTEELLVKLNREDLLRKQRTFDNGSIPHQIHLGELHAI
LRRQEDFYPFLKDNREKIEKILTFRIPYYVGPLARGNSRFAWMTR
KSEETITPWNFEEVVDKGASAQSFIERMTNFDKNLPNEKVLPKHS
LLYEYFTVYNELTKVKYVTEGMRKPAFLSGEQKKAIVDLLFKTNR
KVTVKQLKEDYFKKIECFDSVEISGVEDRFNASLGTYHDLLKIIK
DKDFLDNEENEDILEDIVLTLTLFEDREMIEERLKTYAHLFDDKV
MKQLKRRRYTGWGRLSRKLINGIRDKQSGKTILDFLKSDGFANRN
FMQLIHDDSLTFKEDIQKAQVSGQGDSLHEHIANLAGSPAIKKGI
LQTVKVVDELVKVMGRHKPENIVIEMARENQTTQKGQKNSRERMK
RIEEGIKELGSQILKEHPVENTQLQNEKLYLYYLQNGRDMYVDQE
LDINRLSDYDVDHIVPQSFLKDDSIDNKVLTRSDKNRGKSDNVPS
EEVVKKMKNYWRQLLNAKLITQRKFDNLTKAERGGLSELDKAGFI
KRQLVETRQITKHVAQILDSRMNTKYDENDKLIREVKVITLKSKL
VSDFRKDFQFYKVREINNYHHAHDAYLNAVVGTALIKKYPKLESE
FVYGDYKVYDVRKMIAKSEQEIGKATAKYFFYSNIMNFFKTEITL
ANGEIRKRPLIETNGETGEIVWDKGRDFATVRKVLSMPQVNIVKK
TEVQTGGFSKESILPKRNSDKLIARKKDWDPKKYGGFDSPTVAYS
VLVVAKVEKGKSKKLKSVKELLGITIMERSSFEKNPIDFLEAKGY
KEVKKDLIIKLPKYSLFELENGRKRMLASAGELQKGNELALPSKY
VNFLYLASHYEKLKGSPEDNEQKQLFVEQHKHYLDEIIEQISEFS
KRVILADANLDKVLSAYNKHRDKPIREQAENIIHLFTLTNLGAPA
AFKYFDTTIDRKRYTSTKEVLDATLIHQSITGLYETRIDLSQLGG
DSGGSPKKKRKV
pNMG-623
ecTadA_{(wild-type)}-32 a.a. linker-
ecTadA_{(W23L}_H36L_P48A_R51L_L84F_A106V_
_{D108N}_H123Y_S146C_
_{D147Y}_R152PE155V_1156F_K157N)-
24 a.a. linker nCas9 GGS _NLS
(SEQ ID NO: 195)
MSEVEFSHEYWMRHALTLAKRAWDEREVPVGAVLVHNNRVIGEGW
NRPIGRHDPTAHAEIMALRQGGLVMQNYRLIDATLYVTLEPCVMC
AGAMIHSRIGRVVFGARDAKTGAAGSLMDVLHHPGMNHRVEITEG
ILADECAALLSDFFRMRRQEIKAQKKAQSSTDSGGSSGGSSGSET
PGTSESATPESSGGSSGGSSEVEFSHEYWMRHALTLAKRALDERE
VPVGAVLVLNNRVIGEGWNRAIGLHDPTAHAEIMALRQGGLVMQN
YRLIDATLYVTFEPCVMCAGAMIHSRIGRVVFGVRNAKTGAAGSL
MDVLHYPGMNHRVEITEGILADECAALLCYFFRMPRQVFNAQKKA
QSSTDSGGSSGGSSGSETPGTSESATPESDKKYSIGLAIGTNSVG
WAVITDEYKVPSKKFKVLGNTDRHSIKKNLIGALLFDSGETAEAT
RLKRTARRRYTRRKNRICYLQEIFSNEMAKVDDSFFHRLEESFLV
EEDKKHERHPIFGNIVDEVAYHEKYPTIYHLRKKLVDSTDKADLR
LIYLALAHMIKFRGHFLIEGDLNPDNSDVDKLFIQLVQTYNQLFE
ENPINASGVDAKAILSARLSKSRRLENLIAQLPGEKKNGLFGNLI
ALSLGLTPNFKSNFDLAEDAKLQLSKDTYDDDLDNLLAQIGDQYA
DLFLAAKNLSDAILLSDILRVNTEITKAPLSASMIKRYDEHHQDL
TLLKALVRQQLPEKYKEIFFDQSKNGYAGYIDGGASQEEFYKFIK
PILEKMDGTEELLVKLNREDLLRKQRTFDNGSIPHQIHLGELHAI
LRRQEDFYPFLKDNREKIEKILTFRIPYYVGPLARGNSRFAWMTR
KSEETITPWNFEEVVDKGASAQSFIERMTNFDKNLPNEKVLPKHS
LLYEYFTVYNELTKVKYVTEGMRKPAFLSGEQKKAIVDLLFKTNR
KVTVKQLKEDYFKKIECFDSVEISGVEDRFNASLGTYHDLLKIIK
DKDFLDNEENEDILEDIVLTLTLFEDREMIEERLKTYAHLFDDKV
MKQLKRRRYTGWGRLSRKLINGIRDKQSGKTILDFLKSDGFANRN
FMQLIHDDSLTFKEDIQKAQVSGQGDSLHEHIANLAGSPAIKKGI
LQTVKVVDELVKVMGRHKPENIVIEMARENQTTQKGQKNSRERMK
RIEEGIKELGSQILKEHPVENTQLQNEKLYLYYLQNGRDMYVDQE
LDINRLSDYDVDHIVPQSFLKDDSIDNKVLTRSDKNRGKSDNVPS
EEVVKKMKNYWRQLLNAKLITQRKFDNLTKAERGGLSELDKAGFI
KRQLVETRQITKHVAQILDSRMNTKYDENDKLIREVKVITLKSKL
VSDFRKDFQFYKVREINNYHHAHDAYLNAVVGTALIKKYPKLESE
FVYGDYKVYDVRKMIAKSEQEIGKATAKYFFYSNIMNFFKTEITL
ANGEIRKRPLIETNGETGEIVWDKGRDFATVRKVLSMPQVNIVKK
TEVQTGGFSKESILPKRNSDKLIARKKDWDPKKYGGFDSPTVAYS
VLVVAKVEKGKSKKLKSVKELLGITIMERSSFEKNPIDFLEAKGY
KEVKKDLIIKLPKYSLFELENGRKRMLASAGELQKGNELALPSKY
VNFLYLASHYEKLKGSPEDNEQKQLFVEQHKHYLDEIIEQISEFS
KRVILADANLDKVLSAYNKHRDKPIREQAENIIHLFTLTNLGAPA
AFKYFDTTIDRKRYTSTKEVLDATLIHQSITGLYETRIDLSQLGG
DSGGSPKKKRKV
ABE6.3
ecTadA_{(wild-type)}-(SGGS)₂-XTEN-(SGGS)₂-
ecTadA_{(H36L}_P48S_R51L_L84F_A106V_
_{D108N}_H123Y_S146C_D147Y_E155V_I156F_K157N)-
(SGGS)₂-XTEN-(SGGS)₂_nCas9_SGGS_NLS
(SEQ ID NO: 196)
MSEVEFSHEYWMRHALTLAKRAWDEREVPVGAVLVHNNRVIGEGW
NRPIGRHDPTAHAEIMALRQGGLVMQNYRLIDATLYVTLEPCVMC
AGAMIHSRIGRVVFGARDAKTGAAGSLMDVLHHPGMNHRVEITEG
ILADECAALLSDFFRMRRQEIKAQKKAQSSTDSGGSSGGSSGSET
PGTSESATPESSGGSSGGSSEVEFSHEYWMRHALTLAKRAWDERE
VPVGAVLVLNNRVIGEGWNRSIGLHDPTAHAEIMALRQGGLVMQN
YRLIDATLYVTFEPCVMCAGAMIHSRIGRVVFGVRNAKTGAAGSL
MDVLHYPGMNHRVEITEGILADECAALLCYFFRMRRQVFNAQKKA
QSSTDSGGSSGGSSGSETPGTSESATPESSGGSSGGSDKKYSIGL
AIGTNSVGWAVITDEYKVPSKKFKVLGNTDRHSIKKNLIGALLFD
SGETAEATRLKRTARRRYTRRKNRICYLQEIFSNEMAKVDDSFFH
RLEESFLVEEDKKHERHPIFGNIVDEVAYHEKYPTIYHLRKKLVD
STDKADLRLIYLALAHMIKFRGHFLIEGDLNPDNSDVDKLFIQLV
QTYNQLFEENPINASGVDAKAILSARLSKSRRLENLIAQLPGEKK
NGLFGNLIALSLGLTPNFKSNFDLAEDAKLQLSKDTYDDDLDNLL
AQIGDQYADLFLAAKNLSDAILLSDILRVNTEITKAPLSASMIKR
YDEHHQDLTLLKALVRQQLPEKYKEIFFDQSKNGYAGYIDGGASQ
EEFYKFIKPILEKMDGTEELLVKLNREDLLRKQRTFDNGSIPHQI
HLGELHAILRRQEDFYPFLKDNREKIEKILTFRIPYYVGPLARGN
SRFAWMTRKSEETITPWNFEEVVDKGASAQSFIERMTNFDKNLPN
EKVLPKHSLLYEYFTVYNELTKVKYVTEGMRKPAFLSGEQKKAIV
DLLFKTNRKVTVKQLKEDYFKKIECFDSVEISGVEDRFNASLGTY
HDLLKIIKDKDFLDNEENEDILEDIVLTLTLFEDREMIEERLKTY
AHLFDDKVMKQLKRRRYTGWGRLSRKLINGIRDKQSGKTILDFLK
SDGFANRNFMQLIHDDSLTFKEDIQKAQVSGQGDSLHEHIANLAG
SPAIKKGILQTVKVVDELVKVMGRHKPENIVIEMARENQTTQKGQ
KNSRERMKRIEEGIKELGSQILKEHPVENTQLQNEKLYLYYLQNG
RDMYVDQELDINRLSDYDVDHIVPQSFLKDDSIDNKVLTRSDKNR
GKSDNVPSEEVVKKMKNYWRQLLNAKLITQRKFDNLTKAERGGLS
ELDKAGFIKRQLVETRQITKHVAQILDSRMNTKYDENDKLIREVK
VITLKSKLVSDFRKDFQFYKVREINNYHHAHDAYLNAVVGTALIK
KYPKLESEFVYGDYKVYDVRKMIAKSEQEIGKATAKYFFYSNIMN
FFKTEITLANGEIRKRPLIETNGETGEIVWDKGRDFATVRKVLSM
PQVNIVKKTEVQTGGFSKESILPKRNSDKLIARKKDWDPKKYGGF
DSPTVAYSVLVVAKVEKGKSKKLKSVKELLGITIMERSSFEKNPI
DFLEAKGYKEVKKDLIIKLPKYSLFELENGRKRMLASAGELQKGN
ELALPSKYVNFLYLASHYEKLKGSPEDNEQKQLFVEQHKHYLDEH
IEQISEFSKRVILADANLDKVLSAYNKHRDKPIREQAENIIHLFT
LTNLGAPAAFKYFDTTIDRKRYTSTKEVLDATLIHQSITGLYETR
IDLSQLGGD
SGGSPKKKRKV
ABE6.4
ecTadA_{(wild-type)}-(SGGS)₂-XTEN-(SGGS)₂-
ecTadA_{(H36L}_P48S_R51L_L84F_A106V_
_{D108N}_H123Y_A142N_S146C_D147Y_E155V_
_{I156F}_K157N)⁻(SGGS)₂-XTEN-(SGGS)₂_
nCas9_SGGS_NLS
(SEQ ID NO: 197)
MSEVEFSHEYWMRHALTLAKRAWDEREVPVGAVLVHNNRVIGEGW
NRPIGRHDPTAHAEIMALRQGGLVMQNYRLIDATLYVTLEPCVMC
AGAMIHSRIGRVVFGARDAKTGAAGSLMDVLHHPGMNHRVEITEG
ILADECAALLSDFFRMRRQEIKAQKKAQSSTDSGGSSGGSSGSET
PGTSESATPESSGGSSGGSSEVEFSHEYWMRHALTLAKRAWDERE
VPVGAVLVLNNRVIGEGWNRSIGLHDPTAHAEIMALRQGGLVMQN
YRLIDATLYVTFEPCVMCAGAMIHSRIGRVVFGVRNAKTGAAGSL
MDVLHYPGMNHRVEITEGILADECNALLCYFFRMRRQVFNAQKKA
QSSTDSGGSSGGSSGSETPGTSESATPESSGGSSGGSDKKYSIGL
AIGTNSVGWAVITDEYKVPSKKFKVLGNTDRHSIKKNLIGALLFD
SGETAEATRLKRTARRRYTRRKNRICYLQEIFSNEMAKVDDSFFH
RLEESFLVEEDKKHERHPIFGNIVDEVAYHEKYPTIYHLRKKLVD
STDKADLRLIYLALAHMIKFRGHFLIEGDLNPDNSDVDKLFIQLV
QTYNQLFEENPINASGVDAKAILSARLSKSRRLENLIAQLPGEKK
NGLFGNLIALSLGLTPNFKSNFDLAEDAKLQLSKDTYDDDLDNLL
AQIGDQYADLFLAAKNLSDAILLSDILRVNTEITKAPLSASMIKR
YDEHHQDLTLLKALVRQQLPEKYKEIFFDQSKNGYAGYIDGGASQ
EEFYKFIKPILEKMDGTEELLVKLNREDLLRKQRTFDNGSIPHQI
HLGELHAILRRQEDFYPFLKDNREKIEKILTFRIPYYVGPLARGN
SRFAWMTRKSEETITPWNFEEVVDKGASAQSFIERMTNFDKNLPN
EKVLPKHSLLYEYFTVYNELTKVKYVTEGMRKPAFLSGEQKKAIV
DLLFKTNRKVTVKQLKEDYFKKIECFDSVEISGVEDRFNASLGTY
HDLLKIIKDKDFLDNEENEDILEDIVLTLTLFEDREMIEERLKTY
AHLFDDKVMKQLKRRRYTGWGRLSRKLINGIRDKQSGKTILDFLK
SDGFANRNFMQLIHDDSLTFKEDIQKAQVSGQGDSLHEHIANLAG
SPAIKKGILQTVKVVDELVKVMGRHKPENIVIEMARENQTTQKGQ
KNSRERMKRIEEGIKELGSQILKEHPVENTQLQNEKLYLYYLQNG
RDMYVDQELDINRLSDYDVDHIVPQSFLKDDSIDNKVLTRSDKNR
GKSDNVPSEEVVKKMKNYWRQLLNAKLITQRKFDNLTKAERGGLS
ELDKAGFIKRQLVETRQITKHVAQILDSRMNTKYDENDKLIREVK
VITLKSKLVSDFRKDFQFYKVREINNYHHAHDAYLNAVVGTALIK
KYPKLESEFVYGDYKVYDVRKMIAKSEQEIGKATAKYFFYSNIMN
FFKTEITLANGEIRKRPLIETNGETGEIVWDKGRDFATVRKVLSM
PQVNIVKKTEVQTGGFSKESILPKRNSDKLIARKKDWDPKKYGGF
DSPTVAYSVLVVAKVEKGKSKKLKSVKELLGITIMERSSFEKNPI
DFLEAKGYKEVKKDLIIKLPKYSLFELENGRKRMLASAGELQKGN
ELALPSKYVNFLYLASHYEKLKGSPEDNEQKQLFVEQHKHYLDEH
IEQISEFSKRVILADANLDKVLSAYNKHRDKPIREQAENIIHLFT
LTNLGAPAAFKYFDTTIDRKRYTSTKEVLDATLIHQSITGLYETR
IDLSQLGGDSGGSPKKKRKV
ABE7.8
ecTadA_{(wild-type)}-(SGGS)₂-XTEN-(SGGS)₂-
ecTadA_{(W23L}_H36L_P48A_R51L_L84F_A106V_D108N_
_{H123Y}_A142N_S146C_D147Y_E155V_I156F_K157N)-
(SGGS)-XTEN-(SGGS)₂_nCas9_SGGS_NLS
(SEQ ID NO: 198)
MSEVEFSHEYWMRHALTLAKRAWDEREVPVGAVLVHNNRVIGEGW
NRPIGRHDPTAHAEIMALRQGGLVMQNYRLIDATLYVTLEPCVMC
AGAMIHSRIGRVVFGARDAKTGAAGSLMDVLHHPGMNHRVEITEG
ILADECAALLSDFFRMRRQEIKAQKKAQSSTDSGGSSGGSSGSET
PGTSESATPESSGGSSGGSSEVEFSHEYWMRHALTLAKRALDERE
VPVGAVLVLNNRVIGEGWNRAIGLHDPTAHAEIMALRQGGLVMQN
YRLIDATLYVTFEPCVMCAGAMIHSRIGRVVFGVRNAKTGAAGSL
MDVLHYPGMNHRVEITEGILADECNALLCYFFRMRRQVFNAQKKA
QSSTDSGGSSGGSSGSETPGTSESATPESSGGSSGGSDKKYSIGL
AIGTNSVGWAVITDEYKVPSKKFKVLGNTDRHSIKKNLIGALLFD
SGETAEATRLKRTARRRYTRRKNRICYLQEIFSNEMAKVDDSFFH
RLEESFLVEEDKKHERHPIFGNIVDEVAYHEKYPTIYHLRKKLVD
STDKADLRLIYLALAHMIKFRGHFLIEGDLNPDNSDVDKLFIQLV
QTYNQLFEENPINASGVDAKAILSARLSKSRRLENLIAQLPGEKK
NGLFGNLIALSLGLTPNFKSNFDLAEDAKLQLSKDTYDDDLDNLL
AQIGDQYADLFLAAKNLSDAILLSDILRVNTEITKAPLSASMIKR
YDEHHQDLTLLKALVRQQLPEKYKEIFFDQSKNGYAGYIDGGASQ
EEFYKFIKPILEKMDGTEELLVKLNREDLLRKQRTFDNGSIPHQI
HLGELHAILRRQEDFYPFLKDNREKIEKILTFRIPYYVGPLARGN
SRFAWMTRKSEETITPWNFEEVVDKGASAQSFIERMTNFDKNLPN
EKVLPKHSLLYEYFTVYNELTKVKYVTEGMRKPAFLSGEQKKAIV
DLLFKTNRKVTVKQLKEDYFKKIECFDSVEISGVEDRFNASLGTY
HDLLKIIKDKDFLDNEENEDILEDIVLTLTLFEDREMIEERLKTY
AHLFDDKVMKQLKRRRYTGWGRLSRKLINGIRDKQSGKTILDFLK
SDGFANRNFMQLIHDDSLTFKEDIQKAQVSGQGDSLHEHIANLAG
SPAIKKGILQTVKVVDELVKVMGRHKPENIVIEMARENQTTQKGQ
KNSRERMKRIEEGIKELGSQILKEHPVENTQLQNEKLYLYYLQNG
RDMYVDQELDINRLSDYDVDHIVPQSFLKDDSIDNKVLTRSDKNR
GKSDNVPSEEVVKKMKNYWRQLLNAKLITQRKFDNLTKAERGGLS
ELDKAGFIKRQLVETRQITKHVAQILDSRMNTKYDENDKLIREVK
VITLKSKLVSDFRKDFQFYKVREINNYHHAHDAYLNAVVGTALIK
KYPKLESEFVYGDYKVYDVRKMIAKSEQEIGKATAKYFFYSNIMN
FFKTEITLANGEIRKRPLIETNGETGEIVWDKGRDFATVRKVLSM
PQVNIVKKTEVQTGGFSKESILPKRNSDKLIARKKDWDPKKYGGF
DSPTVAYSVLVVAKVEKGKSKKLKSVKELLGITIMERSSFEKNPI
DFLEAKGYKEVKKDLIIKLPKYSLFELENGRKRMLASAGELQKGN
ELALPSKYVNFLYLASHYEKLKGSPEDNEQKQLFVEQHKHYLDEI
IEQISEFSKRVILADANLDKVLSAYNKHRDKPIREQAENIIHLFT
LTNLGAPAAFKYFDTTIDRKRYTSTKEVLDATLIHQSITGLYETR
IDLSQLGGDSGGSPKKKRKVc
ABE7.9
ecTadA_{(wild-type)}-(SGGS)₂-XTEN-(SGGS)₂-
ecTadA_{(W23L}_H36L_P48A_R51L_L84F_A106V_
_{D108N}_H123Y_A142N_S146C_D147Y_R152P_
_{E155V}_1156F_K157N)-(SGGS)₂-XTEN-
(SGGS)₂_nCas9_SGGS_NLS
(SEQ ID NO: 199)
MSEVEFSHEYWMRHALTLAKRAWDEREVPVGAVLVHNNRVIGEGW
NRPIGRHDPTAHAEIMALRQGGLVMQNYRLIDATLYVTLEPCVMC
AGAMIHSRIGRVVFGARDAKTGAAGSLMDVLHHPGMNHRVEITEG
ILADECAALLSDFFRMRRQEIKAQKKAQSSTDSGGSSGGSSGSET
PGTSESATPESSGGSSGGSSEVEFSHEYWMRHALTLAKRALDERE
VPVGAVLVLNNRGEGWNRAIGLHDPTAHAEIMALRQGGLVMQNYR
LIDATLYVTFEPCVMCAGAMIHSRIGRVVFGVRNAKTGAAGSLMD
VLHYPGMNHRVEITEGILADECNALLCYFFRMPRQVFNAQKKAQS
STDSGGSSGGSSGSETPGTSESATPESSGGSSGGSDKKYSIGLAI
GTNSVGWAVITDEYKVPSKKFKVLGNTDRHSIKKNLIGALLFDSG
ETAEATRLKRTARRRYTRRKNRICYLQEIFSNEMAKVDDSFFHRL
EESFLVEEDKKHERHPIFGNIVDEVAYHEKYPTIYHLRKKLVDST
DKADLRLIYLALAHMIKFRGHFLIEGDLNPDNSDVDKLFIQLVQT
YNQLFEENPINASGVDAKAILSARLSKSRRLENLIAQLPGEKKNG
LFGNLIALSLGLTPNFKSNFDLAEDAKLQLSKDTYDDDLDNLLAQ
IGDQYADLFLAAKNLSDAILLSDILRVNTEITKAPLSASMIKRYD
EHHQDLTLLKALVRQQLPEKYKEIFFDQSKNGYAGYIDGGASQEE
FYKFIKPILEKMDGTEELLVKLNREDLLRKQRTFDNGSIPHQIHL
GELHAILRRQEDFYPFLKDNREKIEKILTFRIPYYVGPLARGNSR
FAWMTRKSEETITPWNFEEVVDKGASAQSFIERMTNFDKNLPNEK
VLPKHSLLYEYFTVYNELTKVKYVTEGMRKPAFLSGEQKKAIVDL
LFKTNRKVTVKQLKEDYFKKIECFDSVEISGVEDRFNASLGTYHD
LLKIIKDKDFLDNEENEDILEDIVLTLTLFEDREMIEERLKTYAH
LFDDKVMKQLKRRRYTGWGRLSRKLINGIRDKQSGKTILDFLKSD
GFANRNFMQLIHDDSLTFKEDIQKAQVSGQGDSLHEHIANLAGSP
AIKKGILQTVKVVDELVKVMGRHKPENIVIEMARENQTTQKGQKN
SRERMKRIEEGIKELGSQILKEHPVENTQLQNEKLYLYYLQNGRD
MYVDQELDINRLSDYDVDHIVPQSFLKDDSIDNKVLTRSDKNRGK
SDNVPSEEVVKKMKNYWRQLLNAKLITQRKFDNLTKAERGGLSEL
DKAGFIKRQLVETRQITKHVAQILDSRMNTKYDENDKLIREVKVI
TLKSKLVSDFRKDFQFYKVREINNYHHAHDAYLNAVVGTALIKKY
PKLESEFVYGDYKVYDVRKMIAKSEQEIGKATAKYFFYSNIMNFF
KTEITLANGEIRKRPLIETNGETGEIVWDKGRDFATVRKVLSMPQ
VNIVKKTEVQTGGFSKESILPKRNSDKLIARKKDWDPKKYGGFDS
PTVAYSVLVVAKVEKGKSKKLKSVKELLGITIMERSSFEKNPIDF
LEAKGYKEVKKDLIIKLPKYSLFELENGRKRMLASAGELQKGNEL
ALPSKYVNFLYLASHYEKLKGSPEDNEQKQLFVEQHKHYLDEIIE
QISEFSKRVILADANLDKVLSAYNKHRDKPIREQAENIIHLFTLT
NLGAPAAFKYFDTTIDRKRYTSTKEVLDATLIHQSITGLYETRID
LSQLGGDSGGSPKKKRKV
ABE7.10
ecTadA_{(wild-type)}-(SGGS)₂-XTEN-(SGGS)₂-
ecTadA_{(W23R}_H36L_P48A_R51L_L84F_A106V_
_{D108N}_H123Y_S146C_D147Y_R152P_E155V_
_{I156F}_K157N)-(SGGS)₂-XTEN-(SGGS)₂_
nCas9_SGGS_NLS
(SEQ ID NO: 200)
MSEVEFSHEYWMRHALTLAKRAWDEREVPVGAVLVHNNRVIGEGW
NRPIGRHDPTAHAEIMALRQGGLVMQNYRLIDATLYVTLEPCVMC
AGAMIHSRIGRVVFGARDAKTGAAGSLMDVLHHPGMNHRVEITEG
ILADECAALLSDFFRMRRQEIKAQKKAQSSTDSGGSSGGSSGSET
PGTSESATPESSGGSSGGSSEVEFSHEYWMRHALTLAKRARDERE
VPVGAVLVLNNRVIGEGWNRAIGLHDPTAHAEIMALRQGGLVMQN
YRLIDATLYVTFEPCVMCAGAMIHSRIGRVVFGVRNAKTGAAGSL
MDVLHYPGMNHRVEITEGILADECAALLCYFFRMPRQVFNAQKKA
QSSTDSGGSSGGSSGSETPGTSESATPESSGGSSGGSDKKYSIGL
AIGTNSVGWAVITDEYKVPSKKFKVLGNTDRHSIKKNLIGALLFD
SGETAEATRLKRTARRRYTRRKNRICYLQEIFSNEMAKVDDSFFH
RLEESFLVEEDKKHERHPIFGNIVDEVAYHEKYPTIYHLRKKLVD
STDKADLRLIYLALAHMIKFRGHFLIEGDLNPDNSDVDKLFIQLV
QTYNQLFEENPINASGVDAKAILSARLSKSRRLENLIAQLPGEKK
NGLFGNLIALSLGLTPNFKSNFDLAEDAKLQLSKDTYDDDLDNLL
AQIGDQYADLFLAAKNLSDAILLSDILRVNTEITKAPLSASMIKR
YDEHHQDLTLLKALVRQQLPEKYKEIFFDQSKNGYAGYIDGGASQ
EEFYKFIKPILEKMDGTEELLVKLNREDLLRKQRTFDNGSIPHQI
HLGELHAILRRQEDFYPFLKDNREKIEKILTFRIPYYVGPLARGN
SRFAWMTRKSEETITPWNFEEVVDKGASAQSFIERMTNFDKNLPN
EKVLPKHSLLYEYFTVYNELTKVKYVTEGMRKPAFLSGEQKKAIV
DLLFKTNRKVTVKQLKEDYFKKIECFDSVEISGVEDRFNASLGTY
HDLLKIIKDKDFLDNEENEDILEDIVLTLTLFEDREMIEERLKTY
AHLFDDKVMKQLKRRRYTGWGRLSRKLINGIRDKQSGKTILDFLK
SDGFANRNFMQLIHDDSLTFKEDIQKAQVSGQGDSLHEHIANLAG
SPAIKKGILQTVKVVDELVKVMGRHKPENIVIEMARENQTTQKGQ
KNSRERMKRIEEGIKELGSQILKEHPVENTQLQNEKLYLYYLQNG
RDMYVDQELDINRLSDYDVDHIVPQSFLKDDSIDNKVLTRSDKNR
GKSDNVPSEEVVKKMKNYWRQLLNAKLITQRKFDNLTKAERGGLS
ELDKAGFIKRQLVETRQITKHVAQILDSRMNTKYDENDKLIREVK
VITLKSKLVSDFRKDFQFYKVREINNYHHAHDAYLNAVVGTALIK
KYPKLESEFVYGDYKVYDVRKMIAKSEQEIGKATAKYFFYSNIMN
FFKTEITLANGEIRKRPLIETNGETGEIVWDKGRDFATVRKVLSM
PQVNTVKKTEVQTGGFSKESILPKRNSDKLIARKKDWDPKKYGGF
DSPTVAYSVLVVAKVEKGKSKKLKSVKELLGITIMERSSFEKNPI
DFLEAKGYKEVKKDLIIKLPKYSLFELENGRKRMLASAGELQKGN
ELALPSKYVNFLYLASHYEKLKGSPEDNEQKQLFVEQHKHYLDEH
IEQISEFSKRVILADANLDKVLSAYNKHRDKPIREQAENIIHLFT
LTNLGAPAAFKYFDTTIDRKRYTSTKEVLDATLIHQSITGLYETR
IDLSQLGGDSGGSPKKKRKV
ABEmax(7.10)
NLS_ecTadA_{(wild-type)}-(SGGS)-XTEN-(SGGS)₂-
ecTadA7.10_{(W23R H36L}_P48A_R51L_
_{L84F}_A106V_D108N_H123Y_S146C_D147Y_R152P_
_{E155V}_I156F_K157N)-(SGGS)₂-XTEN-(SGGS)₂_nCas9
VRQR_SGGS_NLS
(SEQ ID NO: 201)
MKRTADGSEFESPKKKRKVSEVEFSHEYWMRHALTLAKRAWDERE
VPVGAVLVHNNRVIGEGWNRPIGRHDPTAHAEIMALRQGGLVMQN
YRLIDATLYVTLEPCVMCAGAMIHSRIGRVVFGARDAKTGAAGSL
MDVLHHPGMNHRVEITEGILADECAALLSDFFRMRRQEIKAQKKA
QSSTDSGGSSGGSSGSETPGTSESATPESSGGSSGGSSEVEFSHE
YWMRHALTLAKRARDEREVPVGAVLVLNNRVIGEGWNRAIGLHDP
TAHAEIMALRQGGLVMQNYRLIDATLYVTFEPCVMCAGAMIHSRI
GRVVFGVRNAKTGAAGSLMDVLHYPGMNHRVEITEGILADECAAL
LCYFFRMPRQVFNAQKKAQSSTDSGGSSGGSSGSETPGTSESATP
ESSGGSSGGSDKKYSIGLAIGTNSVGWAVITDEYKVPSKKFKVLG
NTDRHSIKKNLIGALLFDSGETAEATRLKRTARRRYTRRKNRICY
LQEIFSNEMAKVDDSFFHRLEESFLVEEDKKHERHPIFGNIVDEV
AYHEKYPTIYHLRKKLVDSTDKADLRLIYLALAHMIKFRGHFLIE
GDLNPDNSDVDKLFIQLVQTYNQLFEENPINASGVDAKAILSARL
SKSRRLENLIAQLPGEKKNGLFGNLIALSLGLTPNFKSNFDLAED
AKLQLSKDTYDDDLDNLLAQIGDQYADLFLAAKNLSDAILLSDIL
RVNTEITKAPLSASMIKRYDEHHQDLTLLKALVRQQLPEKYKEIF
FDQSKNGYAGYIDGGASQEEFYKFIKPILEKMDGTEELLVKLNRE
DLLRKQRTFDNGSIPHQIHLGELHAILRRQEDFYPFLKDNREKIE
KILTFRIPYYVGPLARGNSRFAWMTRKSEETITPWNFEEVVDKGA
SAQSFIERMTNFDKNLPNEKVLPKHSLLYEYFTVYNELTKVKYVT
EGMRKPAFLSGEQKKAIVDLLFKTNRKVTVKQLKEDYFKKIECFD
SVEISGVEDRFNASLGTYHDLLKIIKDKDFLDNEENEDILEDIVL
TLTLFEDREMIEERLKTYAHLFDDKVMKQLKRRRYTGWGRLSRKL
INGIRDKQSGKTILDFLKSDGFANRNFMQLIHDDSLTFKEDIQKA
QVSGQGDSLHEHIANLAGSPAIKKGILQTVKVVDELVKVMGRHKP
ENIVIEMARENQTTQKGQKNSRERMKRIEEGIKELGSQILKEHPV
ENTQLQNEKLYLYYLQNGRDMYVDQELDINRLSDYDVDHIVPQSF
LKDDSIDNKVLTRSDKNRGKSDNVPSEEVVKKMKNYWRQLLNAKL
ITQRKFDNLTKAERGGLSELDKAGFIKRQLVETRQITKHVAQILD
SRMNTKYDENDKLIREVKVITLKSKLVSDFRKDFQFYKVREINNY
HHAHDAYLNAVVGTALIKKYPKLESEFVYGDYKVYDVRKMIAKSE
QEIGKATAKYFFYSNIMNFFKTEITLANGEIRKRPLIETNGETGE
IVWDKGRDFATVRKVLSMPQVNIVKKTEVQTGGFSKESILPKRNS
DKLIARKKDWDPKKYGGFVSPTVAYSVLVVAKVEKGKSKKLKSVK
ELLGITIMERSSFEKNPIDFLEAKGYKEVKKDLIIKLPKYSLFEL
ENGRKRMLASARELQKGNELALPSKYVNFLYLASHYEKLKGSPED
NEQKQLFVEQHKHYLDEIIEQISEFSKRVILADANLDKVLSAYNK
HRDKPIREQAENIIHLFTLTNLGAPAAFKYFDTTIDRKQYRSTKE
VLDATLIHQSITGLYETRIDLSQLGGD
SGGSKRTADGSEFEPKKKRKV
Exemplary base editors comprise sequences
that are at least least 85%, at least 90%,
at least 95%, at least 98%, at least 99%,
or at least 99.5% identical to any of the
following amino acid sequences:
ABEZe
(SEQ ID NO: 202)
MKRTADGSEFESPKKKRKVSEVEFSHEYWMRHALTLAKRARDERE
VPVGAVLVLNNRVIGEGWNRAIGLHDPTAHAEIMALRPGGLVMPN
YRLIDATLYVTFEPCVMCAGAMIHSRIGRVVFGVRNSKRGAAGSL
MNVLNYPGMNHRVEITEGILADECAALLCDFYRMPRPVFNAPKKA
PSSINSGGSSGGSSGSETPGTSESATPESSGGSSGGSDKKYSIGL
AIGTNSVGWAVITDEYKVPSKKFKVLGNTDRHSIKKNLIGALLFD
SGETAEATRLKRTARRRYTRRKNRICYLPEIFSNEMAKVDDSFFH
RLEESFLVEEDKKHERHPIFGNIVDEVAYHEKYPTIYHLRKKLVD
STDKADLRLIYLALAHMIKFRGHFLIEGDLNPDNSDVDKLFIPLV
PTYNPLFEENPINASGVDAKAILSARLSKSRRLENLIAPLPGEKK
NGLFGNLIALSLGLTPNFKSNFDLAEDAKLPLSKDTYDDDLDNLL
APIGDPYADLFLAAKNLSDAILLSDILRVNTEITKAPLSASMIKR
YDEHHPDLTLLKALVRPPLPEKYKEIFFDPSKNGYAGYIDGGASP
EEFYKFIKPILEKMDGTEELLVKLNREDLLRKPRTFDNGSIPHPI
HLGELHAILRRPEDFYPFLKDNREKIEKILTFRIPYYVGPLARGN
SRFAWMTRKSEETITPWNFEEVVDKGASAPSFIERMTNFDKNLPN
EKVLPKHSLLYEYFTVYNELTKVKYVTEGMRKPAFLSGEPKKAIV
DLLFKTNRKVTVKPLKEDYFKKIECFDSVEISGVEDRFNASLGTY
HDLLKIIKDKDFLDNEENEDILEDIVLTLTLFEDREMIEERLKTY
AHLFDDKVMKPLKRRRYTGWGRLSRKLINGIRDKPSGKTILDFLK
SDGFANRNFMPLIHDDSLTFKEDIPKAPVSGPGDSLHEHIANLAG
SPAIKKGILPTVKVVDELVKVMGRHKPENIVIEMARENPTTPKGP
KNSRERMKRIEEGIKELGSPILKEHPVENTPLPNEKLYLYYLQNG
RDMYVDQELDINRLSDYDVDHTVPQSFLKDDSIDNKVLTRSDKNR
GKSDNVPSEEVVKKMKNYWRQLLNAKLITQRKFDNLTKAERGGLS
ELDKAGFIKRQLVETRQITKHVAQILDSRMNTKYDENDKLIREVK
VITLKSKLVSDFRKDFQFYKVREINNYHHAHDAYLNAVVGTALIK
KYPKLESEFVYGDYKVYDVRKMIAKSEQEIGKATAKYFFYSNIMN
FFKTEITLANGEIRKRPLIETNGETGEIVWDKGRDFATVRKVLSM
PQVNIVKKTEVQTGGFSKESILPKRNSDKLIARKKDWDPKKYGGF
DSPTVAYSVLVVAKVEKGKSKKLKSVKELLGITIMERSSFEKNPI
DFLEAKGYKEVKKDLIIKLPKYSLFELENGRKRMLASAGELQKGN
ELALPSKYVNFLYLASHYEKLKGSPEDNEQKQLFVEQHKHYLDEI
IEQISEFSKRVILADANLDKVLSAYNKHRDKPIREQAENIIHLFT
LTNLGAPAAFKYFDTTIDRKRYTSTKEVLDATLIHQSITGLYETR
IDLSQLGGDSGGSKRTADGSEFEPKKKRKV
ABESe-dimer
(SEQ ID NO: 203)
MKRTADGSEFESPKKKRKVSEVEFSHEYWMRHALTLAKRAWDERE
VPVGAVLVHNNRVIGEGWNRPIGRHDPTAHAEIMALRQGGLVMQN
YRLIDATLYVTLEPCVMCAGAMIHSRIGRVVFGARDAKTGAAGSL
MDVLHHPGMNHRVEITEGILADECAALLSDFFRMRRQEIKAQKKA
QSSTDSGGSSGGSSGSETPGTSESATPESSGGSSGGSSEVEFSHE
YWMRHALTLAKRARDEREVPVGAVLVLNNRVIGEGWNRAIGLHDP
TAHAEIMALRQGGLVMQNYRLIDATLYVTFEPCVMCAGAMIHSRI
GRVVFGVRNSKRGAAGSLMNVLNYPGMNHRVEITEGILADECAAL
LCDFYRMPRQVFNAQKKAQSSINSGGSSGGSSGSETPGTSESATP
ESSGGSSGGSDKKYSIGLAIGTNSVGWAVITDEYKVPSKKFKVLG
NTDRHSIKKNLIGALLFDSGETAEATRLKRTARRRYTRRKNRICY
LQEIFSN
EMAKVDDSFFHRLEESFLVEEDKKHERHPIFGNIVDEVAYHEKYP
TIYHLRKKLVDSTDKADLRLIYLALAHMIKFRGHFLIEGDLNPDN
SDVDKLFIQLVQTYNQLFEENPINASGVDAKAILSARLSKSRRLE
NLIAQLPGEKKNGLFGNLIALSLGLTPNFKSNFDLAEDAKLQLSK
DTYDDDLDNLLAQIGDQYADLFLAAKNLSDAILLSDILRVNTEIT
KAPLSASMIKRYDEHHQDLTLLKALVRQQLPEKYKEIFFDQSKNG
YAGYIDGGASQEEFYKFIKPILEKMDGTEELLVKLNREDLLRKQR
TFDNGSIPHQIHLGELHAILRRQEDFYPFLKDNREKIEKILTFRI
PYYVGPLARGNSRFAWMTRKSEETITPWNFEEVVDKGASAQSFIE
RMTNFDKNLPNEKVLPKHSLLYEYFTVYNELTKVKYVTEGMRKPA
FLSGEQKKAIVDLLFKTNRKVTVKQLKEDYFKKIECFDSVEISGV
EDRFNASLGTYHDLLKIIKDKDFLDNEENEDILEDIVLTLTLFED
REMIEERLKTYAHLFDDKVMKQLKRRRYTGWGRLSRKLINGIRDK
QSGKTILDFLKSDGFANRNFMQLIHDDSLTFKEDIQKAQVSGQGD
SLHEHIANLAGSPAIKKGILQTVKVVDELVKVMGRHKPENIVIEM
ARENQTTQKGQKNSRERMKRIEEGIKELGSQILKEHPVENTQLQN
EKLYLYYLQNGRDMYVDQELDINRLSDYDVDHIVPQSFLKDDSID
NKVLTRSDKNRGKSDNVPSEEVVKKMKNYWRQLLNAKLITQRKFD
NLTKAERGGLSELDKAGFIKRQLVETRQITKHVAQILDSRMNTKY
DENDKLIREVKVITLKSKLVSDFRKDFQFYKVREINNYHHAHDAY
LNAVVGTALIKKYPKLESEFVYGDYKVYDVRKMIAKSEQEIGKAT
AKYFFYSNIMNFFKTEITLANGEIRKRPLIETNGETGEIVWDKGR
DFATVRKVLSMPQVNIVKKTEVQTGGFSKESILPKRNSDKLIARK
KDWDPKKYGGFDSPTVAYSVLVVAKVEKGKSKKLKSVKELLGITI
MERSSFEKNPIDFLEAKGYKEVKKDLIIKLPKYSLFELENGRKRM
LASAGELQKGNELALPSKYVNFLYLASHYEKLKGSPEDNEQKQLF
VEQHKHYLDEIIEQISEFSKRVILADANLDKVLSAYNKHRDKPIR
EQAENIIHLFTLTNLGAPAAFKYFDTTIDRKRYTSTKEVLDATLI
HQSITGLYETRI
DLSQLGGDSGGSKRTADGSEFEPKKKRKV
SaABE8e
(SEQ ID NO: 204)
MKRTADGSEFESPKKKRKVSEVEFSHEYWMRHALTLAKRARDERE
VPVGAVLVLNNRVIGEGWNRAIGLHDPTAHAEIMALRQGGLVMQN
YRLIDATLYVTFEPCVMCAGAMIHSRIGRVVFGVRNSKRGAAGSL
MNVLNYPGMNHRVEITEGILADECAALLCDFYRMPRQVFNAQKKA
QSSINSGGSSGGSSGSETPGTSESATPESSGGSSGGSGKRNYILG
LAIGITSVGYGIIDYETRDVIDAGVRLFKEANVENNEGRRSKRGA
RRLKRRRRHRIQRVKKLLFDYNLLTDHSELSGINPYEARVKGLSQ
KLSEEEFSAALLHLAKRRGVHNVNEVEEDTGNELSTKEQISRNSK
ALEEKYVAELQLERLKKDGEVRGSINRFKTSDYVKEAKQLLKVQK
AYHQLDQSFIDTYIDLLETRRTYYEGPGEGSPFGWKDIKEWYEML
MGHCTYFPEELRSVKYAYNADLYNALNDLNNLVITRDENEKLEYY
EKFQIIENVFKQKKKPTLKQIAKEILVNEEDIKGYRVTSTGKPEF
TNLKVYHDIKDITARKEIIENAELLDQIAKILTIYQSSEDIQEEL
TNLNSELTQEEIEQISNLKGYTGTHNLSLKAINLILDELWHTNDN
QIAIFNRLKLVPKKVDLSQQKEIPTTLVDDFILSPVVKRSFIQSI
KVINAIIKKYGLPNDIIIELAREKNSKDAQKMINEMQKRNRQTNE
RIEEIIRTTGKENAKYLI
EKIKLHDMQEGKCLYSLEAIPLEDLLNNPFNYEVDHIIPRSVSFD
NSFNNKVLVKQEENSKKGNRTPFQYLSSSDSKISYETFKKHILNL
AKGKGRISKTKKEYLLEERDINRFSVQKDFINRNLVDTRYATRGL
MNLLRSYFRVNNLDVKVKSINGGFTSFLRRKWKFKKERNKGYKHH
AEDALIIANADFIFKEWKKLDKAKKVMENQMFEEKQAESMPEIET
EQEYKEIFITPHQIKHIKDFKDYKYSHRVDKKPNRELINDTLYST
RKDDKGNTLIVNNLNGLYDKDNDKLKKLINKSPEKLLMYHHDPQT
YQKLKLIMEQYGDEKNPLYKYYEETGNYLTKYSKKDNGPVIKKIK
YYGNKLNAHLDITDDYPNSRNKVVKLSLKPYRFDVYLDNGVYKFV
TVKNLDVIKKENYYEVNSKCYEEAKKLKKISNQAEFIASFYNNDL
IKINGELYRVIGVNNDLLNRIEVNMIDITYREYLENMNDKRPPRI
IKTIASKTQSIKKYSTDILGNLYEVKSKKHPQI
IKKGSGGSKRTADGSEFEPKKKRKV
SaABESe-dimer
(SEQ ID NO: 205)
MKRTADGSEFESPKKKRKVSEVEFSHEYWMRHALTLAKRAWDERE
VPVGAVLVHNNRVIGEGWNRPIGRHDPTAHAEIMALRQGGLVMQN
YRLIDATLYVTLEPCVMCAGAMIHSRIGRVVFGARDAKTGAAGSL
MDVLHHPGMNHRVEITEGILADECAALLSDFFRMRRQEIKAQKKA
QSSTDSGGSSGGSSGSETPGTSESATPESSGGSSGGSSEVEFSHE
YWMRHALTLAKRARDEREVPVGAVLVLNNRVIGEGWNRAIGLHDP
TAHAEIMALRQGGLVMQNYRLIDATLYVTFEPCVMCAGAMIHSRI
GRVVFGVRNSKRGAAGSLMNVLNYPGMNHRVEITEGILADECAAL
LCDFYRMPRQVFNAQKKAQSSINSGGSSGGSSGSETPGTSESATP
ESSGGSSGGSGKRNYILGLAIGITSVGYGIIDYETRDVIDAGVRL
FKEANVENNEGRRSKRGARRLKRRRRHRIQRVKKLLFDYNLLTDH
SELSGINPYEARVKGLSQKLSEEEFSAALLHLAKRRGVHNVNEVE
EDTGNELSTKEQISRNSKALEEKYVAELQLERLKKDGEVRGSINR
FKTSDYVKEAKQLLKVQKAYHQLDQSFIDTYIDLLETRRTYYEGP
GEGSPFGWKDIKEWYEMLMGHCTYFPEELRSVKYAYNADLYNALN
DLNNLVITRDENEKLEYYEKFQIIENVFKQKKKPTLKQIAKEILV
NEEDIKGYRVTSTGKPEFTNLKVYHDIKDITARKEIIENAELLDQ
IAKILTIYQSSEDIQEELTNLNSELTQEEIEQISNLKGYTGTHNL
SLKAINLILDELWHTNDNQIAIFNRLKLVPKKVDLSQQKEIPTTL
VDDFILSPVVKRSFIQSIKVINAIIKKYGLPNDIIIELAREKNSK
DAQKMINEMQKRNRQTNERIEEIIRTTGKENAKYLIEKIKLHDMQ
EGKCLYSLEAIPLEDLLNNPFNYEVDHIIPRSVSFDNSFNNKVLV
KQEENSKKGNRTPFQYLSSSDSKISYETFKKHILNLAKGKGRISK
TKKEYLLEERDINRFSVQKDFINRNLVDTRYATRGLMNLLRSYFR
VNNLDVKVKSINGGFTSFLRRKWKFKKERNKGYKHHAEDALIIAN
ADFIFKEWKKLDKAKKVMENQMFEEKQAESMPEIETEQEYKEIFI
TPHQIKHIKDFKDYKYSHRVDKKPNRELINDTLYSTRKDDKGNTL
IVNNLNGLYDKDNDKLKKLINKSPEKLLMYHHDPQTYQKLKLIME
QYGDEKNPLYKYYEETGNYLTKYSKKDNGPVIKKIKYYGNKLNAH
LDITDDYPNSRNKVVKLSLKPYRFDVYLDNGVYKFVTVKNLDVIK
KENYYEVNSKCYEEAKKLKKISNQAEFIASFYNNDLIKINGELYR
VIGVNNDLLNRIEVNMIDITYREYLENMNDKRPPRIIKTIASKTQ
SIKKYSTDILGNLYEVKSKKHPQIIKKGSGGSKRTADGSEFEPKK
KRKV
LbABE8e
(SEQ ID NO: 206)
MKRTADGSEFESPKKKRKVSEVEFSHEYWMRHALTLAKRARDERE
VPVGAVLVLNNRVIGEGWNRAIGLHDPTAHAEIMALRQGGLVMQN
YRLIDATLYVTFEPCVMCAGAMIHSRIGRVVFGVRNSKRGAAGSL
MNVLNYPGMNHRVEITEGILADECAALLCDFYRMPRQVFNAQKKA
QSSINSGGSSGGSSGSETPGTSESATPESSGGSSGGSSKLEKFTN
CYSLSKTLRFKAIPVGKTQENIDNKRLLVEDEKRAEDYKGVKKLL
DRYYLSFINDVLHSIKLKNLNNYISLFRKKTRTEKENKELENLEI
NLRKEIAKAFKGNEGYKSLFKKDIIETILPEFLDDKDEIALVNSF
NGFTTAFTGFFDNRENMFSEEAKSTSIAFRCINENLTRYISNMDI
FEKVDAIFDKHEVQEIKEKILNSDYDVEDFFEGEFFNFVLTQEGI
DVYNAIIGGFVTESGEKIKGLNEYINLYNQKTKQKLPKFKPLYKQ
VLSDRESLSFYGEGYTSDEEVLEVFRNTLNKNSEIFSSIKKLEKL
FKNFDEYSSAGIFVKNGPAISTISKDIFGEWNVIRDKWNAEYDDI
HLKKKAVVTEKYEDDRRKSFKKIGSFSLEQLQEYADADLSVVEKL
KEIIIQKVDEIYKVYGSSEKLFDADFVLEKSLKKNDAVVAIMKDL
LDSVKSFENYIKAFFGEGKETNRDESFYGDFVLAYDILLKVDHIY
DAIRNYVTQKPYSKDKFKLYFQNPQFMGGWDKDKETDYRATILRY
GSKYYLAIMDKKYAKCLQKIDKDDVNGNYEKINYKLLPGPNKMLP
KVFFSKKWMAYYNPSEDIQKIYKNGTFKKGDMFNLNDCHKLIDFF
KDSISRYPKWSNAYDFNFSETEKYKDIAGFYREVEEQGYKVSFES
ASKKEVDKLVEEGKLYMFQIYNKDFSDKSHGTPNLHTMYFKLLFD
ENNHGQIRLSGGAELFMRRASLKKEELVVHPANSPIANKNPDNPK
KTTTLSYDVYKDKRFSEDQYELHIPIAINKCPKNIFKINTEVRVL
LKHDDNPYVIGIARGERNLLYIVVVDGKGNIVEQYSLNEIINNFN
GIRIKTDYHSLLDKKEKERFEARQNWTSIENIKELKAGYISQVVH
KICELVEKYDAVIALEDLNSGFKNSRVKVEKQVYQKFEKMLIDKL
NYMVDKKSNPCATGGALKGYQITNKFESFKSMSTQNGFIFYIPAW
LTSKIDPSTGFVNLLKTKYTSIADSKKFISSFDRIMYVPEEDLFE
FALDYKNFSRTDADYIKKWKLYSYGNRIRIFRNPKKNNVFDWEEV
CLTSAYKELFNKYGINYQQGDIRALLCEQSDKAFYSSFMALMSLM
LQMRNSITGRTDVDFLISPVKNSDGIFYDSRNYEAQENAILPKNA
DANGAYNIARKVLWAIGQFKKAEDEKLDKVKIAISNKEWLEYAQT
SVKSGGSKRTADGSEFEPKKKRKV
LbABE8e-dimer
(SEQ ID NO: 207)
MKRTADGSEFESPKKKRKVSEVEFSHEYWMRHALTLAKRAWDERE
VPVGAVLVHNNRVIGEGWNRPIGRHDPTAHAEIMALRQGGLVMQN
YRLIDATLYVTLEPCVMCAGAMIHSRIGRVVFGARDAKTGAAGSL
MDVLHHPGMNHRVEITEGILADECAALLSDFFRMRRQEIKAQKKA
QSSTDSGGSSGGSSGSETPGTSESATPESSGGSSGGSSEVEFSHE
YWMRHALTLAKRARDEREVPVGAVLVLNNRVIGEGWNRAIGLHDP
TAHAEIMALRQGGLVMQNYRLIDATLYVTFEPCVMCAGAMIHSRI
GRVVFGVRNSKRGAAGSLMNVLNYPGMNHRVEITEGILADECAAL
LCDFYRMPRQVFNAQKKAQSSINSGGSSGGSSGSETPGTSESATP
ESSGGSSGGSSKLEKFTNCYSLSKTLRFKAIPVGKTQENIDNKRL
LVEDEKRAEDYKGVKKLLDRYYLSFINDVLHSIKLKNLNNYISLF
RKKTRTEKENKELENLEINLRKEIAKAFKGNEGYKSLFKKDIIET
ILPEFLDDKDEIALVNSFNGFTTAFTGFFDNRENMFSEEAKSTSI
AFRCINENLTRYISNMDIFEKVDAIFDKHEVQEIKEKILNSDYDV
EDFFEGEFFNFVLTQEGIDVYNAIIGGFVTESGEKIKGLNEYINL
YNQKTKQKLPKFKPLYKQVLSDRESLSFYGEGYTSDEEVLEVFRN
TLNKNSEIFSSIKKLEKLFKNFDEYSSAGIFVKNGPAISTISKDI
FGEWNVIRDKWNAEYDDIHLKKKAVVTEKYEDDRRKSFKKIGSFS
LEQLQEYADADLSVVEKLKEIIIQKVDEIYKVYGSSEKLFDADFV
LEKSLKKNDAVVAIMKDLLDSVKSFENYIKAFFGEGKETNRDESF
YGDFVLAYDILLKVDHIYDAIRNYVTQKPYSKDKFKLYFQNPQFM
GGWDKDKETDYRATILRYGSKYYLAIMDKKYAKCLQKIDKDDVNG
NYEKINYKLLPGPNKMLPKVFFSKKWMAYYNPSEDIQKIYKNGTF
KKGDMFNLNDCHKLIDFFKDSISRYPKWSNAYDFNFSETEKYKDI
AGFYREVEEQGYKVSFESASKKEVDKLVEEGKLYMFQIYNKDFSD
KSHGTPNLHTMYFKLLFDENNHGQIRLSGGAELFMRRASLKKEEL
VVHPANSPLVNKNPDNPKKTTTLSYDVYKDKRFSEDQYELHIPIA
INKCPKNIFKINTEVRVLLKHDDNPYVIGIARGERNLLYIVVVDG
KGNIVEQYSLNEIINNFNGIRIKTDYHSLLDKKEKERFEARQNWT
SIENIKELKAGYISQVVHKICELVEKYDAVIALEDLNSGFKNSRV
KVEKQVYQKFEKMLIDKLNYMVDKKSNPCATGGALKGYQITNKFE
SFKSMSTQNGFIFYIPAWLTSKIDPSTGFVNLLKTKYTSIADSKK
FISSFDRIMYVPEEDLFEFALDYKNFSRTDADYIKKWKLYSYGNR
IRIFRNPKKNNVFDWEEVCLTSAYKELFNKYGINYQQGDIRALLC
EQSDKAFYSSFMALMSLMLQMRNSITGRTDVDFLISPVKNSDGIF
YDSRNYEAQENAILPKNADANGAYNIARKVLWAIGQFKKAEDEKL
DKVKIAISNKEWLEYAQTSVKSGGSKRTADGSEFEPKKKR
KV
LbABE7.10
(SEQ ID NO: 208)
MKRTADGSEFESPKKKRKVSEVEFSHEYWMRHALTLAKRAWDERE
VPVGAVLVHNNRVIGEGWNRPIGRHDPTAHAEIMALRQGGLVMQN
YRLIDATLYVTLEPCVMCAGAMIHSRIGRVVFGARDAKTGAAGSL
MDVLHHPGMNHRVEITEGILADECAALLSDFFRMRRQEIKAQKKA
QSSTDSGGSSGGSSGSETPGTSESATPESSGGSSGGSSEVEFSHE
YWMRHALTLAKRARDEREVPVGAVLVLNNRVIGEGWNRAIGLHDP
TAHAEIMALRQGGLVMQNYRLIDATLYVTFEPCVMCAGAMIHSRI
GRVVFGVRNAKTGAAGSLMDVLHYPGMNHRVEITEGILADECAAL
LCYFFRMPRQVFNAQKKAQSSTDSGGSSGGSSGSETPGTSESATP
ESSGGSSGGSSKLEKFTNCYSLSKTLRFKAIPVGKTQENIDNKRL
LVEDEKRAEDYKGVKKLLDRYYLSFINDVLHSIKLKNLNNYISLF
RKKTRTEKENKELENLEINLRKEIAKAFKGNEGYKSLFKKDIIET
ILPEFLDDKDEIALVNSFNGFTTAFTGFFDNRENMFSEEAKSTSI
AFRCINENLTRYISNMDIFEKVDAIFDKHEVQEIKEKILNSDYDV
EDFFEGEFFNFVLTQEGIDVYNAIIGGFVTESGEKIKGLNEYINL
YNQKTKQKLPKFKPLYKQVLSDRESLSFYGEGYTSDEEVLEVFRN
TLNKNSEIFSSIKKLEKLFKNFDEYSSAGIFVKNGPAISTISKDI
FGEWNVIRDKWNAEYDDIHLKKKAVVTEKYEDDRRKSFKKIGSFS
LEQLQEYADADLSVVEKLKEIIIQKVDEIYKVYGSSEKLFDADFV
LEKSLKKNDAVVAIMKDLLDSVKSFENYIKAFFGEGKETNRDESF
YGDFVLAYDILLKVDHIYDAIRNYVTQKPYSKDKFKLYFQNPQFM
GGWDKDKETDYRATILRYGSKYYLAIMDKKYAKCLQKIDKDDVNG
NYEKINYKLLPGPNKMLPKVFFSKKWMAYYNPSEDIQKIYKNGTF
KKGDMFNLNDCHKLIDFFKDSISRYPKWSNAYDFNFSETEKYKDI
AGFYREVEEQGYKVSFESASKKEVDKLVEEGKLYMFQIYNKDFSD
KSHGTPNLHTMYFKLLFDENNHGQIRLSGGAELFMRRASLKKEEL
VVHPANSPIANKNPDNPKKTTTLSYDVYKDKRFSEDQYELHIPIA
INKCPKNIFKINTEVRVLLKHDDNPYVIGIARGERNLLYIVVVDG
KGNIVEQYSLNEIINNFNGIRIKTDYHSLLDKKEKERFEARQNWT
SIENIKELKAGYISQVVHKICELVEKYDAVIALEDLNSGFKNSRV
KVEKQVYQKFEKMLIDKLNYMVDKKSNPCATGGALKGYQITNKFE
SFKSMSTQNGFIFYIPAWLTSKIDPSTGFVNLLKTKYTSIADSKK
FISSFDRIMYVPEEDLFEFALDYKNFSRTDADYIKKWKLYSYGNR
IRIFRNPKKNNVFDWEEVCLTSAYKELFNKYGINYQQGDIRALLC
EQSDKAFYSSFMALMSLMLQMRNSITGRTDVDFLISPVKNSDGIF
YDSRNYEAQENAILPKNADANGAYNIARKVLWAIGQFKKAEDEKL
DKVKIAISNKEWLEYAQTSVKSGGSKRTADGSEFEPKKKR
KV
enAsABE8e
(SEQ ID NO: 209)
MKRTADGSEFESPKKKRKVSEVEFSHEYWMRHALTLAKRARDERE
VPVGAVLVLNNRVIGEGWNRAIGLHDPTAHAEIMALRQGGLVMQN
YRLIDATLYVTFEPCVMCAGAMIHSRIGRVVFGVRNSKRGAAGSL
MNVLNYPGMNHRVEITEGILADECAALLCDFYRMPRQVFNAQKKA
QSSINSGGSSGGSSGSETPGTSESATPESSGGSSGGSMTQFEGFT
NLYQVSKTLRFELIPQGKTLKHIQEQGFIEEDKARNDHYKELKPI
IDRIYKTYADQCLQLVQLDWENLSAAIDSYRKEKTEETRNALIEE
QATYRNAIHDYFIGRTDNLTDAINKRHAEIYKGLFKAELFNGKVL
KQLGTVTTTEHENALLRSFDKFTTYFSGFYRNRKNVFSAEDISTA
IPHRIVQDNFPKFKENCHIFTRLITAVPSLREHFENVKKAIGIFV
STSIEEVFSFPFYNQLLTQTQIDLYNQLLGGISREAGTEKIKGLN
EVLNLAIQKNDETAHIIASLPHRFIPLFKQILSDRNTLSFILEEF
KSDEEVIQSFCKYKTLLRNENVLETAEALFNELNSIDLTHIFISH
KKLETISSALCDHWDTLRNALYERRISELTGKITKSAKEKVQRSL
KHEDINLQEIISAAGKELSEAFKQKTSEILSHAHAALDQPLPTTL
KKQEEKEILK
SQLDSLLGLYHLLDWFAVDESNEVDPEFSARLTGIKLEMEPSLSF
YNKARNYATKKPYSVEKFKLNFQMPTLARGWDVNREKNNGAILFV
KNGLYYLGIMPKQKGRYKALSFEPTEKTSEGFDKMYYDYFPDAAK
MIPKCSTQLKAVTAHFQTHTTPILLSNNFIEPLEITKEIYDLNNP
EKEPKKFQTAYAKKTGDQKGYREALCKWIDFTRDFLSKYTKTTSI
DLSSLRPSSQYKDLGEYYAELNPLLYHISFQRIAEKEIMDAVETG
KLYLFQIYNKDFAKGHHGKPNLHTLYWTGLFSPENLAKTSIKLNG
QAELFYRPKSRMKRMAHRLGEKMLNKKLKDQKTPIPDTLYQELYD
YVNHRLSHDLSDEARALLPNVITKEVSHEIIKDRRFTSDKFFFHV
PITLNYQAANSPSKFNQRVNAYLKEHPETPIIGIARGERNLIYIT
VIDSTGKILEQRSLNTIQQFDYQKKLDNREKERVAARQAWSVVGT
IKDLKQGYLSQVIHEIVDLMIHYQAVVVLENLNFGFKSKRTGIAE
KAVYQQFEKMLIDKLNCLVLKDYPAEKVGGVLNPYQLTDQFTSFA
KMGTQSGFLFYVPAPYTSKIDPLTGFVDPFVWKTIKNHESRKHFL
EGFDFLHYDVKTGDFILHFKMNRNLSFQRGLPGFMPAWDIVFEKN
ETQFDAKGTPFIAGKRIVPVIENHRFTGRYRDLYPANELIALLEE
KGIVFRDGSNILPKLLENDDSHAIDTMVALIRSVLQMRNSNAATG
EDYINSPVRDLNGVCFDSRFQNPEWPMDADANGAYHIALKGQLLL
NHLKESKDLKLQNGISNQDWLAYIQELRNSGGSKRTADGSEFEPK
KKRKV
enAsABESe-dimer
(SEQ ID NO: 210)
MKRTADGSEFESPKKKRKVSEVEFSHEYWMRHALTLAKRAWDERE
VPVGAVLVHNNRVIGEGWNRPIGRHDPTAHAEIMALRQGGLVMQN
YRLIDATLYVTLEPCVMCAGAMIHSRIGRVVFGARDAKTGAAGSL
MDVLHHPGMNHRVEITEGILADECAALLSDFFRMRRQEIKAQKKA
QSSTDSGGSSGGSSGSETPGTSESATPESSGGSSGGSSEVEFSHE
YWMRHALTLAKRARDEREVPVGAVLVLNNRVIGEGWNRAIGLHDP
TAHAEIMALRQGGLVMQNYRLIDATLYVTFEPCVMCAGAMIHSRI
GRVVFGVRNSKRGAAGSLMNVLNYPGMNHRVEITEGILADECAAL
LCDFYRMPRQVFNAQKKAQSSINSGGSSGGSSGSETPGTSESATP
ESSGGSSGGSMTQFEGFTNLYQVSKTLRFELIPQGKTLKHIQEQG
FIEEDKARNDHYKELKPIIDRIYKTYADQCLQLVQLDWENLSAAI
DSYRKEKTEETRNALIEEQATYRNAIHDYFIGRTDNLTDAINKRH
AEIYKGLFKAELFNGKVLKQLGTVTTTEHENALLRSFDKFTTYFS
GFYRNRKNVFSAEDISTAIPHRIVQDNFPKFKENCHIFTRLITAV
PSLREHFENVKKAIGIFVSTSIEEVFSFPFYNQLLTQTQIDLYNQ
LLGGISREAGTEKIKGLNEVLNLAIQKNDETAHIIASLPHRFIPL
FKQILSDRNTLSFILEEFKSDEEVIQSFCKYKTLLRNENVLETAE
ALFNELNSIDLTHIFISHKKLETISSALCDHWDTLRNALYERRIS
ELTGKITKSAKEKVQRSLKHEDINLQEIISAAGKELSEAFKQKTS
EILSHAHAALDQPLPTTLKKQEEKEILKSQLDSLLGLYHLLDWFA
VDESNEVDPEFSARLTGIKLEMEPSLSFYNKARNYATKKPYSVEK
FKLNFQMPTLARGWDVNREKNNGAILFVKNGLYYLGIMPKQKGRY
KALSFEPTEKTSEGFDKMYYDYFPDAAKMIPKCSTQLKAVTAHFQ
THTTPILLSNNFIEPLEITKEIYDLNNPEKEPKKFQTAYAKKTGD
QKGYREALCKWIDFTRDFLSKYTKTTSIDLSSLRPSSQYKDLGEY
YAELNPLLYHISFQRIAEKEIMDAVETGKLYLFQIYNKDFAKGHH
GKPNLHTLYWTGLFSPENLAKTSIKLNGQAELFYRPKSRMKRMAH
RLGEKMLNKKLKDQKTPIPDTLYQELYDYVNHRLSHDLSDEARAL
LPNVITKEVSHEIIKDRRFTSDKFFFHVPITLNYQAANSPSKFNQ
RVNAYLKEHPETPIIGIARGERNLIYITVIDSTGKILEQRSLNTI
QQFDYQKKLDNREKERVAARQAWSVVGTIKDLKQGYLSQVIHEIV
DLMIHYQAVVVLENLNFGFKSKRTGIAEKAVYQQFEKMLIDKLNC
LVLKDYPAEKVGGVLNPYQLTDQFTSFAKMGTQSGFLFYVPAPYT
SKIDPLTGFVDPFVWKTIKNHESRKHFLEGFDFLHYDVKTGDFIL
HFKMNRNLSFQRGLPGFMPAWDIVFEKNETQFDAKGTPFIAGKRI
VPVIENHRFTGRYRDLYPANELIALLEEKGIVFRDGSNILPKLLE
NDDSHAIDTMVALIRSVLQMRNSNAATGEDYINSPVRDLNGVCFD
SRFQNPEWPMDADANGAYHIALKGQLLLNHLKESKDLKLQNGISN
QDWLAYIQELRNSGGSKRTADGSEFEPKKKRKV
enAsABE7.10
(SEQ ID NO: 211)
MKRTADGSEFESPKKKRKVSEVEFSHEYWMRHALTLAKRAWDERE
VPVGAVLVHNNRVIGEGWNRPIGRHDPTAHAEIMALRQGGLVMQN
YRLIDATLYVTLEPCVMCAGAMIHSRIGRVVFGARDAKTGAAGSL
MDVLHHPGMNHRVEITEGILADECAALLSDFFRMRRQEIKAQKKA
QSSTDSGGSSGGSSGSETPGTSESATPESSGGSSGGSSEVEFSHE
YWMRHALTLAKRARDEREVPVGAVLVLNNRVIGEGWNRAIGLHDP
TAHAEIMALRQGGLVMQNYRLIDATLYVTFEPCVMCAGAMIHSRI
GRVVFGVRNAKTGAAGSLMDVLHYPGMNHRVEITEGILADECAAL
LCYFFRMPRQVFNAQKKAQSSTDSGGSSGGSSGSETPGTSESATP
ESSGGSSGGSMTQFEGFTNLYQVSKTLRFELIPQGKTLKHIQEQG
FIEEDKARNDHYKELKPIIDRIYKTYADQCLQLVQLDWENLSAAI
DSYRKEKTEETRNALIEEQATYRNAIHDYFIGRTDNLTDAINKRH
AEIYKGLFKAELFNGKVLKQLGTVTTTEHENALLRSFDKFTTYFS
GFYRNRKNVFSAEDISTAIPHRIVQDNFPKFKENCHIFTRLITAV
PSLREHFENVKKAIGIFVSTSIEEVFSFPFYNQLLTQTQIDLYNQ
LLGGISREAGTEKIKGLNEVLNLAIQKNDETAHIIASLPHRFIPL
FKQILSDRNTLSFILEEFKSDEEVIQSFCKYKTLLRNENVLETAE
ALFNELNSIDLTHIFISHKKLETISSALCDHWDTLRNALYERRIS
ELTGKITKSAKEKVQRSLKHEDINLQEIISAAGKELSEAFKQKTS
EILSHAHAALDQPLPTTLKKQEEKEILKSQLDSLLGLYHLLDWFA
VDESNEVDPEFSARLTGIKLEMEPSLSFYNKARNYATKKPYSVEK
FKLNFQMPTLARGWDVNREKNNGAILFVKNGLYYLGIMPKQKGRY
KALSFEPTEKTSEGFDKMYYDYFPDAAKMIPKCSTQLKAVTAHFQ
THTTPILLSNNFIEPLEITKEIYDLNNPEKEPKKFQTAYAKKTGD
QKGYREALCKWIDFTRDFLSKYTKTTSIDLSSLRPSSQYKDLGEY
YAELNPLLYHISFQRIAEKEIMDAVETGKLYLFQIYNKDFAKGHH
GKPNLHTLYWTGLFSPENLAKTSIKLNGQAELFYRPKSRMKRMAH
RLGEKMLNKKLKDQKTPIPDTLYQELYDYVNHRLSHDLSDEARAL
LPNVITKEVSHEIIKDRRFTSDKFFFHVPITLNYQAANSPSKFNQ
RVNAYLKEHPETPIIGIARGERNLIYITVIDSTGKILEQRSLNTI
QQFDYQKKLDNREKERVAARQAWSVVGTIKDLKQGYLSQVIHEIV
DLMIHYQAVVVLENLNFGFKSKRTGIAEKAVYQQFEKMLIDKLNC
LVLKDYPAEKVGGVLNPYQLTDQFTSFAKMGTQSGFLFYVPAPYT
SKIDPLTGFVDPFVWKTIKNHESRKHFLEGFDFLHYDVKTGDFIL
HFKMNRNLSFQRGLPGFMPAWDIVFEKNETQFDAKGTPFIAGKRI
VPVIENHRFTGRYRDLYPANELIALLEEKGIVFRDGSNILPKLLE
NDDSHAIDTMVALIRSVLQMRNSNAATGEDYINSPVRDLNGVCFD
SRFQNPEWPMDADANGAYHIALKGQLLLNHLKESKDLKLQNGISN
QDWLAYIQELRNSGGSKRTADGSEFEPKKKRKV
SpCas9NG-ABE8e (“NG-ABEZe”)
(SEQ ID NO: 212)
MKRTADGSEFESPKKKRKVSEVEFSHEYWMRHALTLAKRARDERE
VPVGAVLVLNNRVIGEGWNRAIGLHDPTAHAEIMALRQGGLVMQN
YRLIDATLYVTFEPCVMCAGAMIHSRIGRVVFGVRNSKRGAAGSL
MNVLNYPGMNHRVEITEGILADECAALLCDFYRMPRQVFNAQKKA
QSSINSGGSSGGSSGSETPGTSESATPESSGGSSGGSDKKYSIGL
AIGTNSVGWAVITDEYKVPSKKFKVLGNTDRHSIKKNLIGALLFD
SGETAEATRLKRTARRRYTRRKNRICYLQEIFSNEMAKVDDSFFH
RLEESFLVEEDKKHERHPIFGNIVDEVAYHEKYPTIYHLRKKLVD
STDKADLRLIYLALAHMIKFRGHFLIEGDLNPDNSDVDKLFIQLV
QTYNQLFEENPINASGVDAKAILSARLSKSRRLENLIAQLPGEKK
NGLFGNLIALSLGLTPNFKSNFDLAEDAKLQLSKDTYDDDLDNLL
AQIGDQYADLFLAAKNLSDAILLSDILRVNTEITKAPLSASMIKR
YDEHHQDLTLLKALVRQQLPEKYKEIFFDQSKNGYAGYIDGGASQ
EEFYKFIKPILEKMDGTEELLVKLNREDLLRKQRTFDNGSIPHQI
HLGELHAILRRQEDFYPFLKDNREKIEKILTFRIPYYVGPLARGN
SRFAWMTRKSEETITPWNFEEVVDKGASAQSFIERMTNFDKNLPN
EKVLPKHSLLYEYFTVYNELTKVKYVTEGMRKPAFLSGEQKKAIV
DLLFKTNRKVTVKQLKEDYFKKIECFDSVEISGVEDRFNASLGTY
HDLLKIIKDKDFLDNEENEDILEDIVLTLTLFEDREMIEERLKTY
AHLFDDKVMKQLKRRRYTGWGRLSRKLINGIRDKQSGKTILDFLK
SDGFANRNFMQLIHDDSLTFKEDIQKAQVSGQGDSLHEHIANLAG
SPAIKKGILQTVKVVDELVKVMGRHKPENIVIEMARENQTTQKGQ
KNSRERMKRIEEGIKELGSQILKEHPVENTQLQNEKLYLYYLQNG
RDMYVDQELDINRLSDYDVDHIVPQSFLKDDSIDNKVLTRSDKNR
GKSDNVPSEEVVKKMKNYWRQLLNAKLITQRKFDNLTKAERGGLS
ELDKAGFIKRQLVETRQITKHVAQILDSRMNTKYDENDKLIREVK
VITLKSKLVSDFRKDFQFYKVREINNYHHAHDAYLNAVVGTALIK
KYPKLESEFVYGDYKVYDVRKMIAKSEQEIGKATAKYFFYSNIMN
FFKTEITLANGEIRKRPLIETNGETGEIVWDKGRDFATVRKVLSM
PQVNIVKKTEVQTGGFSKESIRPKRNSDKLIARKKDWDPKKYGGF
VSPTVAYSVLVVAKVEKGKSKKLKSVKELLGITIMERSSFEKNPI
DFLEAKGYKEVKKDLIIKLPKYSLFELENGRKRMLASARFLQKGN
ELALPSKYVNFLYLASHYEKLKGSPEDNEQKQLFVEQHKHYLDEI
IEQISEFSKRVILADANLDKVLSAYNKHRDKPIREQAENIIHLFT
LTNLGAPRAFKYFDTTIDRKVYRSTKEVLDATLIHQSITGLYETR
IDLSQLGGDSGGSKRTADGSEFEPKKKRKV
NG-ABE8e-dimer
(SEQ ID NO: 213)
MKRTADGSEFESPKKKRKVSEVEFSHEYWMRHALTLAKRAWDERE
VPVGAVLVHNNRVIGEGWNRPIGRHDPTAHAEIMALRQGGLVMQN
YRLIDATLYVTLEPCVMCAGAMIHSRIGRVVFGARDAKTGAAGSL
MDVLHHPGMNHRVEITEGILADECAALLSDFFRMRRQEIKAQKKA
QSSTDSGGSSGGSSGSETPGTSESATPESSGGSSGGSSEVEFSHE
YWMRHALTLAKRARDEREVPVGAVLVLNNRVIGEGWNRAIGLHDP
TAHAEIMALRQGGLVMQNYRLIDATLYVTFEPCVMCAGAMIHSRI
GRVVFGVRNSKRGAAGSLMNVLNYPGMNHRVEITEGILADECAAL
LCDFYRMPRQVFNAQKKAQSSINSGGSSGGSSGSETPGTSESATP
ESSGGSSGGSDKKYSIGLAIGTNSVGWAVITDEYKVPSKKFKVLG
NTDRHSIKKNLIGALLFDSGETAEATRLKRTARRRYTRRKNRICY
LQEIFSN
EMAKVDDSFFHRLEESFLVEEDKKHERHPIFGNIVDEVAYHEKYP
TIYHLRKKLVDSTDKADLRLIYLALAHMIKFRGHFLIEGDLNPDN
SDVDKLFIQLVQTYNQLFEENPINASGVDAKAILSARLSKSRRLE
NLIAQLPGEKKNGLFGNLIALSLGLTPNFKSNFDLAEDAKLQLSK
DTYDDDLDNLLAQIGDQYADLFLAAKNLSDAILLSDILRVNTEIT
KAPLSASMIKRYDEHHQDLTLLKALVRQQLPEKYKEIFFDQSKNG
YAGYIDGGASQEEFYKFIKPILEKMDGTEELLVKLNREDLLRKQR
TFDNGSIPHQIHLGELHAILRRQEDFYPFLKDNREKIEKILTFRI
PYYVGPLARGNSRFAWMTRKSEETITPWNFEEVVDKGASAQSFIE
RMTNFDKNLPNEKVLPKHSLLYEYFTVYNELTKVKYVTEGMRKPA
FLSGEQKKAIVDLLFKTNRKVTVKQLKEDYFKKIECFDSVEISGV
EDRFNASLGTYHDLLKIIKDKDFLDNEENEDILEDIVLTLTLFED
REMIEERLKTYAHLFDDKVMKQLKRRRYTGWGRLSRKLINGIRDK
QSGKTILDFLKSDGFANRNFMQLIHDDSLTFKEDIQKAQVSGQGD
SLHEHIANLAGSPAIKKGILQTVKVVDELVKVMGRHKPENIVIEM
ARENQTTQKGQKNSRERMKRIEEGIKELGSQILKEHPVENTQLQN
EKLYLYYLQNGRDMYVDQELDINRLSDYDVDHIVPQSFLKDDSID
NKVLTRSDKNRGKSDNVPSEEVVKKMKNYWRQLLNAKLITQRKFD
NLTKAERGGLSELDKAGFIKRQLVETRQITKHVAQILDSRMNTKY
DENDKLIREVKVITLKSKLVSDFRKDFQFYKVREINNYHHAHDAY
LNAVVGTALIKKYPKLESEFVYGDYKVYDVRKMIAKSEQEIGKAT
AKYFFYSNIMNFFKTEITLANGEIRKRPLIETNGETGEIVWDKGR
DFATVRKVLSMPQVNIVKKTEVQTGGFSKESIRPKRNSDKLIARK
KDWDPKKYGGFVSPTVAYSVLVVAKVEKGKSKKLKSVKELLGITI
MERSSFEKNPIDFLEAKGYKEVKKDLIIKLPKYSLFELENGRKRM
LASARFLQKGNELALPSKYVNFLYLASHYEKLKGSPEDNEQKQLF
VEQHKHYLDEIIEQISEFSKRVILADANLDKVLSAYNKHRDKPIR
EQAENIIHLFTLTNLGAPRAFKYFDTTIDRKVYRSTKEVLDATLI
HQSITGLYETRI
DLSQLGGDSGGSKRTADGSEFEPKKKRKV
SaKKH-ABESe (“KKH-ABE8e”)
(SEQ ID NO: 214)
MKRTADGSEFESPKKKRKVSEVEFSHEYWMRHALTLAKRARDERE
VPVGAVLVLNNRVIGEGWNRAIGLHDPTAHAEIMALRQGGLVMQN
YRLIDATLYVTFEPCVMCAGAMIHSRIGRVVFGVRNSKRGAAGSL
MNVLNYPGMNHRVEITEGILADECAALLCDFYRMPRQVFNAQKKA
QSSINSGGSSGGSSGSETPGTSESATPESSGGSSGGSGKRNYILG
LAIGITSVGYGIIDYETRDVIDAGVRLFKEANVENNEGRRSKRGA
RRLKRRRRHRIQRVKKLLFDYNLLTDHSELSGINPYEARVKGLSQ
KLSEEEFSAALLHLAKRRGVHNVNEVEEDTGNELSTKEQISRNSK
ALEEKYVAELQLERLKKDGEVRGSINRFKTSDYVKEAKQLLKVQK
AYHQLDQSFIDTYIDLLETRRTYYEGPGEGSPFGWKDIKEWYEML
MGHCTYFPEELRSVKYAYNADLYNALNDLNNLVITRDENEKLEYY
EKFQIIENVFKQKKKPTLKQIAKEILVNEEDIKGYRVTSTGKPEF
TNLKVYHDIKDITARKEIIENAELLDQIAKILTIYQSSEDIQEEL
TNLNSELTQEEIEQISNLKGYTGTHNLSLKAINLILDELWHTNDN
QIAIFNRLKLVPKKVDLSQQKEIPTTLVDDFILSPVVKRSFIQSI
KVINAIIKKYGLPNDIIIELAREKNSKDAQKMINEMQKRNRQTNE
RIEEIIRTTGKENAKYLIEKIKLHDMQEGKCLYSLEAIPLEDLLN
NPFNYEVDHIIPRSVSFDNSFNNKVLVKQEENSKKGNRTPFQYLS
SSDSKISYETFKKHILNLAKGKGRISKTKKEYLLEERDINRFSVQ
KDFINRNLVDTRYATRGLMNLLRSYFRVNNLDVKVKSINGGFTSF
LRRKWKFKKERNKGYKHHAEDALIIANADFIFKEWKKLDKAKKVM
ENQMFEEKQAESMPEIETEQEYKEIFITPHQIKHIKDFKDYKYSH
RVDKKPNRELINDTLYSTRKDDKGNTLIVNNLNGLYDKDNDKLKK
LINKSPEKLLMYHHDPQTYQKLKLIMEQYGDEKNPLYKYYEETGN
YLTKYSKKDNGPVIKKIKYYGNKLNAHLDITDDYPNSRNKVVKLS
LKPYRFDVYLDNGVYKFVTVKNLDVIKKENYYEVNSKCYEEAKKL
KKISNQAEFIASFYNNDLIKINGELYRVIGVNNDLLNRIEVNMID
ITYREYLENMNDKRPPRIIKTIASKTQSIKKYSTDILGNLYEVKS
KKHPQI
IKKGSGGSKRTADGSEFEPKKKRKV
SaKKH-ABESe-dimer
(SEQ ID NO: 215)
MKRTADGSEFESPKKKRKVSEVEFSHEYWMRHALTLAKRAWDERE
VPVGAVLVHNNRVIGEGWNRPIGRHDPTAHAEIMALRQGGLVMQN
YRLIDATLYVTLEPCVMCAGAMIHSRIGRVVFGARDAKTGAAGSL
MDVLHHPGMNHRVEITEGILADECAALLSDFFRMRRQEIKAQKKA
QSSTDSGGSSGGSSGSETPGTSESATPESSGGSSGGSSEVEFSHE
YWMRHALTLAKRARDEREVPVGAVLVLNNRVIGEGWNRAIGLHDP
TAHAEIMALRQGGLVMQNYRLIDATLYVTFEPCVMCAGAMIHSRI
GRVVFGVRNSKRGAAGSLMNVLNYPGMNHRVEITEGILADECAAL
LCDFYRMPRQVFNAQKKAQSSINSGGSSGGSSGSETPGTSESATP
ESSGGSSGGSGKRNYILGLAIGITSVGYGIIDYETRDVIDAGVRL
FKEANVENNEGRRSKRGARRLKRRRRHRIQRVKKLLFDYNLLTDH
SELSGINPYEARVKGLSQKLSEEEFSAALLHLAKRRGVHNVNEVE
EDTGNELSTKEQISRNSKALEEKYVAELQLERLKKDGEVRGSINR
FKTSDYVKEAKQLLKVQKAYHQLDQSFIDTYIDLLETRRTYYEGP
GEGSPFGWKDIKEWYEMLMGHCTYFPEELRSVKYAYNADLYNALN
DLNNLVITRDENEKLEYYEKFQIIENVFKQKKKPTLKQIAKEILV
NEEDIKGYRVTSTGKPEFTNLKVYHDIKDITARKEIIENAELLDQ
IAKILTIYQSSEDIQEELTNLNSELTQEEIEQISNLKGYTGTHNL
SLKAINLILDELWHTNDNQIAIFNRLKLVPKKVDLSQQKEIPTTL
VDDFILSPVVKRSFIQSIKVINAIIKKYGLPNDIIIELAREKNSK
DAQKMINEMQKRNRQTNERIEEIIRTTGKENAKYLIEKIKLHDMQ
EGKCLYSLEAIPLEDLLNNPFNYEVDHIIPRSVSFDNSFNNKVLV
KQEENSKKGNRTPFQYLSSSDSKISYETFKKHILNLAKGKGRISK
TKKEYLLEERDINRFSVQKDFINRNLVDTRYATRGLMNLLRSYFR
VNNLDVKVKSINGGFTSFLRRKWKFKKERNKGYKHHAEDALIIAN
ADFIFKEWKKLDKAKKVMENQMFEEKQAESMPEIETEQEYKEIFI
TPHQIKHIKDFKDYKYSHRVDKKPNRELINDTLYSTRKDDKGNTL
IVNNLNGLYDKDNDKLKKLINKSPEKLLMYHHDPQTYQKLKLIME
QYGDEKNPLYKYYEETGNYLTKYSKKDNGPVIKKIKYYGNKLNAH
LDITDDYPNSRNKVVKLSLKPYRFDVYLDNGVYKFVTVKNLDVIK
KENYYEVNSKCYEEAKKLKKISNQAEFIASFYNNDLIKINGELYR
VIGVNNDLLNRIEVNMIDITYREYLENMNDKRPPRIIKTIASKTQ
SIKKYSTDILGNLYEVKSKKHPQIIKKGSGGSKRTADGSEFEPKK
KRKV
CP1028-ABE8e
(SEQ ID NO: 216)
MKRTADGSEFESPKKKRKVSEVEFSHEYWMRHALTLAKRARDERE
VPVGAVLVLNNRVIGEGWNRAIGLHDPTAHAEIMALRQGGLVMQN
YRLIDATLYVTFEPCVMCAGAMIHSRIGRVVFGVRNSKRGAAGSL
MNVLNYPGMNHRVEITEGILADECAALLCDFYRMPRQVFNAQKKA
QSSINSGGSSGGSSGSETPGTSESATPESSGGSSGGSEIGKATAK
YFFYSNIMNFFKTEITLANGEIRKRPLIETNGETGEIVWDKGRDF
ATVRKVLSMPQVNIVKKTEVQTGGFSKESILPKRNSDKLIARKKD
WDPKKYGGFDSPTVAYSVLVVAKVEKGKSKKLKSVKELLGITIME
RSSFEKNPIDFLEAKGYKEVKKDLIIKLPKYSLFELENGRKRMLA
SAGELQKGNELALPSKYVNFLYLASHYEKLKGSPEDNEQKQLFVE
QHKHYLDEIIEQISEFSKRVILADANLDKVLSAYNKHRDKPIREQ
AENIIHLFTLTNLGAPAAFKYFDTTIDRKRYTSTKEVLDATLIHQ
SITGLYETRIDLSQLGGDGGSGGSGGSGGSGGSGGSGGMDKKYSI
GLAIGTNSVGWAVITDEYKVPSKKFKVLGNTDRHSIKKNLIGALL
FDSGETAEATRLKRTARRRYTRRKNRICYLQEIFSNEMAKVDDSF
FHRLEESFLVEEDKKHERHPIFGNIVDEVAYHEKYPTIYHLRKKL
VDSTDKADLRLIYLALAHMIKFRGHFLIEGDLNPDNSDVDKLFIQ
LVQTYNQLFEENPINASGVDAKAILSARLSKSRRLENLIAQLPGE
KKNGLFGNLIALSLGLTPNFKSNFDLAEDAKLQLSKDTYDDDLDN
LLAQIGDQYADLFLAAKNLSDAILLSDILRVNTEITKAPLSASMI
KRYDEHHQDLTLLKALVRQQLPEKYKEIFFDQSKNGYAGYIDGGA
SQEEFYKFIKPILEKMDGTEELLVKLNREDLLRKQRTFDNGSIPH
QIHLGELHAILRRQEDFYPFLKDNREKIEKILTFRIPYYVGPLAR
GNSRFAWMTRKSEETITPWNFEEVVDKGASAQSFIERMTNFDKNL
PNEKVLPKHSLLYEYFTVYNELTKVKYVTEGMRKPAFLSGEQKKA
IVDLLFKTNRKVTVKQLKEDYFKKIECFDSVEISGVEDRFNASLG
TYHDLLKIIKDKDFLDNEENEDILEDIVLTLTLFEDREMIEERLK
TYAHLFDDKVMKQLKRRRYTGWGRLSRKLINGIRDKQSGKTILDF
LKSDGFANRNFMQLIHDDSLTFKEDIQKAQVSGQGDSLHEHIANL
AGSPAIKKGILQTVKVVDELVKVMGRHKPENIVIEMARENQTTQK
GQKNSRERMKRIEEGIKELGSQILKEHPVENTQLQNEKLYLYYLQ
NGRDMYVDQELDINRLSDYDVDHIVPQSFLKDDSIDNKVLTRSDK
NRGKSDNVPSEEVVKKMKNYWRQLLNAKLITQRKFDNLTKAERGG
LSELDKAGFIKRQLVETRQITKHVAQILDSRMNTKYDENDKLIRE
VKVITLKSKLVSDFRKDFQFYKVREINNYHHAHDAYLNAVVGTAL
IKKYPKLESEFVYGDYKVYDVRKMIAKSEQ
SGGSKRTADGSEFEPKKKRKV
CP1028-ABE8e-dimer
(SEQ ID NO: 217)
MKRTADGSEFESPKKKRKVSEVEFSHEYWMRHALTLAKRAWDERE
VPVGAVLVHNNRVIGEGWNRPIGRHDPTAHAEIMALRQGGLVMQN
YRLIDATLYVTLEPCVMCAGAMIHSRIGRVVFGARDAKTGAAGSL
MDVLHHPGMNHRVEITEGILADECAALLSDFFRMRRQEIKAQKKA
QSSTDSGGSSGGSSGSETPGTSESATPESSGGSSGGSSEVEFSHE
YWMRHALTLAKRARDEREVPVGAVLVLNNRVIGEGWNRAIGLHDP
TAHAEIMALRQGGLVMQNYRLIDATLYVTFEPCVMCAGAMIHSRI
GRVVFGVRNSKRGAAGSLMNVLNYPGMNHRVEITEGILADECAAL
LCDFYRMPRQVFNAQKKAQSSINSGGSSGGSSGSETPGTSESATP
ESSGGSSGGSEIGKATAKYFFYSNIMNFFKTEITLANGEIRKRPL
IETNGETGEIVWDKGRDFATVRKVLSMPQVNIVKKTEVQTGGFSK
ESILPKRNSDKLIARKKDWDPKKYGGFDSPTVAYSVLVVAKVEKG
KSKKLKSVKELLGITIMERSSFEKNPIDFLEAKGYKEVKKDLIIK
LPKYSLFELENGRKRMLASAGELQKGNELALPSKYVNFLYLASHY
EKLKGSPEDNEQKQLFVEQHKHYLDEIIEQISEFSKRVILADANL
DKVLSAYNKHRDKPIREQAENIIHLFTLTNLGAPAAFKYFDTTID
RKRYTSTKEVLDATLIHQSITGLYETRIDLSQLGGDGGSGGSGGS
GGSGGSGGSGGMDKKYSIGLAIGTNSVGWAVITDEYKVPSKKFKV
LGNTDRHSIKKNLIGALLFDSGETAEATRLKRTARRRYTRRKNRI
CYLQEIFSNEMAKVDDSFFHRLEESFLVEEDKKHERHPIFGNIVD
EVAYHEKYPTIYHLRKKLVDSTDKADLRLIYLALAHMIKFRGHFL
IEGDLNPDNSDVDKLFIQLVQTYNQLFEENPINASGVDAKAILSA
RLSKSRRLENLIAQLPGEKKNGLFGNLIALSLGLTPNFKSNFDLA
EDAKLQLSKDTYDDDLDNLLAQIGDQYADLFLAAKNLSDAILLSD
ILRVNTEITKAPLSASMIKRYDEHHQDLTLLKALVRQQLPEKYKE
IFFDQSKNGYAGYIDGGASQEEFYKFIKPILEKMDGTEELLVKLN
REDLLRKQRTFDNGSIPHQIHLGELHAILRRQEDFYPFLKDNREK
IEKILTFRIPYYVGPLARGNSRFAWMTRKSEETITPWNFEEVVDK
GASAQSFIERMTNFDKNLPNEKVLPKHSLLYEYFTVYNELTKVKY
VTEGMRKPAFLSGEQKKAIVDLLFKTNRKVTVKQLKEDYFKKIEC
FDSVEISGVEDRFNASLGTYHDLLKIIKDKDFLDNEENEDILEDI
VLTLTLFEDREMIEERLKTYAHLFDDKVMKQLKRRRYTGWGRLSR
KLINGIRDKQSGKTILDFLKSDGFANRNFMQLIHDDSLTFKEDIQ
KAQVSGQGDSLHEHIANLAGSPAIKKGILQTVKVVDELVKVMGRH
KPENIVIEMARENQTTQKGQKNSRERMKRIEEGIKELGSQILKEH
PVENTQLQNEKLYLYYLQNGRDMYVDQELDINRLSDYDVDHIVPQ
SFLKDDSIDNKVLTRSDKNRGKSDNVPSEEVVKKMKNYWRQLLNA
KLITQRKFDNLTKAERGGLSELDKAGFIKRQLVETRQITKHVAQI
LDSRMNTKYDENDKLIREVKVITLKSKLVSDFRKDFQFYKVREIN
NYHHAHDAYLNAVVGTALIKKYPKLESEFVYGDYKVYDVRKMIAK
SEQSGGSKRTADGSEFEPKKKRKV
CP1041-ABE8e
(SEQ ID NO: 218)
MKRTADGSEFESPKKKRKVSEVEFSHEYWMRHALTLAKRARDERE
VPVGAVLVLNNRVIGEGWNRAIGLHDPTAHAEIMALRQGGLVMQN
YRLIDATLYVTFEPCVMCAGAMIHSRIGRVVFGVRNSKRGAAGSL
MNVLNYPGMNHRVEITEGILADECAALLCDFYRMPRQVFNAQKKA
QSSINSGGSSGGSSGSETPGTSESATPESSGGSSGGSNIMNFFKT
EITLANGEIRKRPLIETNGETGEIVWDKGRDFATVRKVLSMPQVN
IVKKTEVQTGGFSKESILPKRNSDKLIARKKDWDPKKYGGFDSPT
VAYSVLVVAKVEKGKSKKLKSVKELLGITIMERSSFEKNPIDFLE
AKGYKEVKKDLIIKLPKYSLFELENGRKRMLASAGELQKGNELAL
PSKYVNFLYLASHYEKLKGSPEDNEQKQLFVEQHKHYLDEIIEQI
SEFSKRVILADANLDKVLSAYNKHRDKPIREQAENIIHLFTLTNL
GAPAAFKYFDTTIDRKRYTSTKEVLDATLIHQSITGLYETRIDLS
QLGGDGGSGGSGGSGGSGGSGGSGGDKKYSIGLAIGTNSVGWAVI
TDEYKVPSKKFKVLGNTDRHSIKKNLIGALLFDSGETAEATRLKR
TARRRYTRRKNRICYLQEIFSNEMAKVDDSFFHRLEESFLVEEDK
KHERHPIFGNIVDEVAYHEKYPTIYHLRKKLVDSTDKADLRLIYL
ALAHMIKFRGHFLIEGDLNPDNSDVDKLFIQLVQTYNQLFEENPI
NASGVDAKAILSARLSKSRRLENLIAQLPGEKKNGLFGNLIALSL
GLTPNFKSNFDLAEDAKLQLSKDTYDDDLDNLLAQIGDQYADLFL
AAKNLSDAILLSDILRVNTEITKAPLSASMIKRYDEHHQDLTLLK
ALVRQQLPEKYKEIFFDQSKNGYAGYIDGGASQEEFYKFIKPILE
KMDGTEELLVKLNREDLLRKQRTFDNGSIPHQIHLGELHAILRRQ
EDFYPFLKDNREKIEKILTFRIPYYVGPLARGNSRFAWMTRKSEE
TITPWNFEEVVDKGASAQSFIERMTNFDKNLPNEKVLPKHSLLYE
YFTVYNELTKVKYVTEGMRKPAFLSGEQKKAIVDLLFKTNRKVTV
KQLKEDYFKKIECFDSVEISGVEDRFNASLGTYHDLLKIIKDKDF
LDNEENEDILEDIVLTLTLFEDREMIEERLKTYAHLFDDKVMKQL
KRRRYTGWGRLSRKLINGIRDKQSGKTILDFLKSDGFANRNFMQL
IHDDSLTFKEDIQKAQVSGQGDSLHEHIANLAGSPAIKKGILQTV
KVVDELVKVMGRHKPENIVIEMARENQTTQKGQKNSRERMKRIEE
GIKELGSQILKEHPVENTQLQNEKLYLYYLQNGRDMYVDQELDIN
RLSDYDVDHIVPQSFLKDDSIDNKVLTRSDKNRGKSDNVPSEEVV
KKMKNYWRQLLNAKLITQRKFDNLTKAERGGLSELDKAGFIKRQL
VETRQITKHVAQILDSRMNTKYDENDKLIREVKVITLKSKLVSDF
RKDFQFYKVREINNYHHAHDAYLNAVVGTALIKKYPKLESEFVYG
DYKVYDVRKMIAKSEQEIGKATAKYFFYS
SGGSKRTADGSEFEPKKKRKV
ABE8e (TadA-8e V82G)
(SEQ ID NO: 219)
MKRTADGSEFESPKKKRKVSEVEFSHEYWMRHALTLAKRARDERE
VPVGAVLVLNNRVIGEGWNRAIGLHDPTAHAEIMALRQGGLVMQN
YRLIDATLYVTFEPCVMCAGAMIHSRIGRVVFGVRNSKRGAAGSL
MNVLNYPGMNHRVEITEGILADECAALLCDFYRMPRQVFNAQKKA
QSSINSGGSSGGSSGSETPGTSESATPESSGGSSGGSDKKYSIGL
AIGTNSVGWAVITDEYKVPSKKFKVLGNTDRHSIKKNLIGALLFD
SGETAEATRLKRTARRRYTRRKNRICYLQEIFSNEMAKVDDSFFH
RLEESFLVEEDKKHERHPIFGNIVDEVAYHEKYPTIYHLRKKLVD
STDKADLRLIYLALAHMIKFRGHFLIEGDLNPDNSDVDKLFIQLV
QTYNQLFEENPINASGVDAKAILSARLSKSRRLENLIAQLPGEKK
NGLFGNLIALSLGLTPNFKSNFDLAEDAKLQLSKDTYDDDLDNLL
AQIGDQYADLFLAAKNLSDAILLSDILRVNTEITKAPLSASMIKR
YDEHHQDLTLLKALVRQQLPEKYKEIFFDQSKNGYAGYIDGGASQ
EEFYKFIKPILEKMDGTEELLVKLNREDLLRKQRTFDNGSIPHQI
HLGELHAILRRQEDFYPFLKDNREKIEKILTFRIPYYVGPLARGN
SRFAWMTRKSEETITPWNFEEVVDKGASAQSFIERMTNFDKNLPN
EKVLPKHSLLYEYFTVYNELTKVKYVTEGMRKPAFLSGEQKKAIV
DLLFKTNRKVTVKQLKEDYFKKIECFDSVEISGVEDRFNASLGTY
HDLLKIIKDKDFLDNEENEDILEDIVLTLTLFEDREMIEERLKTY
AHLFDDKVMKQLKRRRYTGWGRLSRKLINGIRDKQSGKTILDFLK
SDGFANRNFMQLIHDDSLTFKEDIQKAQVSGQGDSLHEHIANLAG
SPAIKKGILQTVKVVDELVKVMGRHKPENIVIEMARENQTTQKGQ
KNSRERMKRIEEGIKELGSQILKEHPVENTQLQNEKLYLYYLQNG
RDMYVDQELDINRLSDYDVDHIVPQSFLKDDSIDNKVLTRSDKNR
GKSDNVPSEEVVKKMKNYWRQLLNAKLITQRKFDNLTKAERGGLS
ELDKAGFIKRQLVETRQITKHVAQILDSRMNTKYDENDKLIREVK
VITLKSKLVSDFRKDFQFYKVREINNYHHAHDAYLNAVVGTALIK
KYPKLESEFVYGDYKVYDVRKMIAKSEQEIGKATAKYFFYSNIMN
FFKTEITLANGEIRKRPLIETNGETGEIVWDKGRDFATVRKVLSM
PQVNIVKKTEVQTGGFSKESILPKRNSDKLIARKKDWDPKKYGGF
DSPTVAYSVLVVAKVEKGKSKKLKSVKELLGITIMERSSFEKNPI
DFLEAKGYKEVKKDLIKLPKYSLFELENGRKRMLASAGELQKGNE
LALPSKYVNFLYLASHYEKLKGSPEDNEQKQLFVEQHKHYLDEII
EQISEFSKRVILADANLDKVLSAYNKHRDKPIREQAENIIHLFTL
TNLGAPAAFKYFDTTIDRKRYTSTKEVLDATLIHQSITGLYETRI
DLSQLGGDSGGSKRTADGSEFEPKKKRKV
ABE8e(TadA-8e K20AR21A)
(SEQ ID NO: 220)
MKRTADGSEFESPKKKRKVSEVEFSHEYWMRHALTLAKRARDERE
VPVGAVLVLNNRVIGEGWNRAIGLHDPTAHAEIMALRQGGLVMQN
YRLIDATLYVTFEPCVMCAGAMIHSRIGRVVFGVRNSKRGAAGSL
MNVLNYPGMNHRVEITEGILADECAALLCDFYRMPRQVFNAQKKA
QSSINSGGSSGGSSGSETPGTSESATPESSGGSSGGSDKKYSIGL
AIGTNSVGWAVITDEYKVPSKKFKVLGNTDRHSIKKNLIGALLFD
SGETAEATRLKRTARRRYTRRKNRICYLQEIFSNEMAKVDDSFFH
RLEESFLVEEDKKHERHPIFGNIVDEVAYHEKYPTIYHLRKKLVD
STDKADLRLIYLALAHMIKFRGHFLIEGDLNPDNSDVDKLFIQLV
QTYNQLFEENPINASGVDAKAILSARLSKSRRLENLIAQLPGEKK
NGLFGNLIALSLGLTPNFKSNFDLAEDAKLQLSKDTYDDDLDNLL
AQIGDQYADLFLAAKNLSDAILLSDILRVNTEITKAPLSASMIKR
YDEHHQDLTLLKALVRQQLPEKYKEIFFDQSKNGYAGYIDGGASQ
EEFYKFIKPILEKMDGTEELLVKLNREDLLRKQRTFDNGSIPHQI
HLGELHAILRRQEDFYPFLKDNREKIEKILTFRIPYYVGPLARGN
SRFAWMTRKSEETITPWNFEEVVDKGASAQSFIERMTNFDKNLPN
EKVLPKHSLLYEYFTVYNELTKVKYVTEGMRKPAFLSGEQKKAIV
DLLFKTNRKVTVKQLKEDYFKKIECFDSVEISGVEDRFNASLGTY
HDLLKIIKDKDFLDNEENEDILEDIVLTLTLFEDREMIEERLKTY
AHLFDDKVMKQLKRRRYTGWGRLSRKLINGIRDKQSGKTILDFLK
SDGFANRNFMQLIHDDSLTFKEDIQKAQVSGQGDSLHEHIANLAG
SPAIKKGILQTVKVVDELVKVMGRHKPENIVIEMARENQTTQKGQ
KNSRERMKRIEEGIKELGSQILKEHPVENTQLQNEKLYLYYLQNG
RDMYVDQELDINRLSDYDVDHIVPQSFLKDDSIDNKVLTRSDKNR
GKSDNVPSEEVVKKMKNYWRQLLNAKLITQRKFDNLTKAERGGLS
ELDKAGFIKRQLVETRQITKHVAQILDSRMNTKYDENDKLIREVK
VITLKSKLVSDFRKDFQFYKVREINNYHHAHDAYLNAVVGTALIK
KYPKLESEFVYGDYKVYDVRKMIAKSEQEIGKATAKYFFYSNIMN
FFKTEITLANGEIRKRPLIETNGETGEIVWDKGRDFATVRKVLSM
PQVNIVKKTEVQTGGFSKESILPKRNSDKLIARKKDWDPKKYGGF
DSPTVAYSVLVVAKVEKGKSKKLKSVKELLGITIMERSSFEKNPI
DFLEAKGYKEVKKDLIIKLPKYSLFELENGRKRMLASAGELQKGN
ELALPSKYVNFLYLASHYEKLKGSPEDNEQKQLFVEQHKHYLDEI
IEQISEFSKRVILADANLDKVLSAYNKHRDKPIREQAENIIHLFT
LTNLGAPAAFKYFDTTIDRKRYTSTKEVLDATLIHQSITGLYETR
IDLSQLGGDSGGSKRTADGSEFEPKKKRKV
ABE8e(TadA-8e V106W)
(SEQ ID NO: 3239)
MKRTADGSEFESPKKKRKVSEVEFSHEYWMRHALTLAKRARDERE
VPVGAVLVLNNRVIGEGWNRAIGLHDPTAHAEIMALRQGGLVMQN
YRLIDATLYVTFEPCVMCAGAMIHSRIGRVVFGWRNSKRGAAGSL
MNVLNYPGMNHRVEITEGILADECAALLCDFYRMPRQVFNAQKKA
QSSINSGGSSGGSSGSETPGTSESATPESSGGSSGGSDKKYSIGL
AIGTNSVGWAVITDEYKVPSKKFKVLGNTDRHSIKKNLIGALLFD
SGETAEATRLKRTARRRYTRRKNRICYLQEIFSNEMAKVDDSFFH
RLEESFLVEEDKKHERHPIFGNIVDEVAYHEKYPTIYHLRKKLVD
STDKADLRLIYLALAHMIKFRGHFLIEGDLNPDNSDVDKLFIQLV
QTYNQLFEENPINASGVDAKAILSARLSKSRRLENLIAQLPGEKK
NGLFGNLIALSLGLTPNFKSNFDLAEDAKLQLSKDTYDDDLDNLL
AQIGDQYADLFLAAKNLSDAILLSDILRVNTEITKAPLSASMIKR
YDEHHQDLTLLKALVRQQLPEKYKEIFFDQSKNGYAGYIDGGASQ
EEFYKFIKPILEKMDGTEELLVKLNREDLLRKQRTFDNGSIPHQI
HLGELHAILRRQEDFYPFLKDNREKIEKILTFRIPYYVGPLARGN
SRFAWMTRKSEETITPWNFEEVVDKGASAQSFIERMTNFDKNLPN
EKVLPKHSLLYEYFTVYNELTKVKYVTEGMRKPAFLSGEQKKAIV
DLLFKTNRKVTVKQLKEDYFKKIECFDSVEISGVEDRFNASLGTY
HDLLKIIKDKDFLDNEENEDILEDIVLTLTLFEDREMIEERLKTY
AHLFDDKVMKQLKRRRYTGWGRLSRKLINGIRDKQSGKTILDFLK
SDGFANRNFMQLIHDDSLTFKEDIQKAQVSGQGDSLHEHIANLAG
SPAIKKGILQTVKVVDELVKVMGRHKPENIVIEMARENQTTQKGQ
KNSRERMKRIEEGIKELGSQILKEHPVENTQLQNEKLYLYYLQNG
RDMYVDQELDINRLSDYDVDHIVPQSFLKDDSIDNKVLTRSDKNR
GKSDNVPSEEVVKKMKNYWRQLLNAKLITQRKFDNLTKAERGGLS
ELDKAGFIKRQLVETRQITKHVAQILDSRMNTKYDENDKLIREVK
VITLKSKLVSDFRKDFQFYKVREINNYHHAHDAYLNAVVGTALIK
KYPKLESEFVYGDYKVYDVRKMIAKSEQEIGKATAKYFFYSNIMN
FFKTEITLANGEIRKRPLIETNGETGEIVWDKGRDFATVRKVLSM
PQVNIVKKTEVQTGGFSKESILPKRNSDKLIARKKDWDPKKYGGF
DSPTVAYSVLVVAKVEKGKSKKLKSVKELLGITIMERSSFEKNPI
DFLEAKGYKEVKKDLIIKLPKYSLFELENGRKRMLASAGELQKGN
ELALPSKYVNFLYLASHYEKLKGSPEDNEQKQLFVEQHKHYLDEI
IEQISEFSKRVILADANLDKVLSAYNKHRDKPIREQAENIIHLFT
LTNLGAPAAFKYFDTTIDRKRYTSTKEVLDATLIHQSITGLYETR
IDLSQLGGDSGGSKRTADGSEFEPKKKRKV
ABE8e-NRTH dimer editor: NLS, wtTadA,
linker, TadA*, SpCas9-NRTH
(SEQ ID NO: 3240)
MKRTADGSEFESPKKKRKVSEVEFSHEYWMRHALTLAKRAWDERE
VPVGAVLVHNNRVIGEGWNRPIGRHDPTAHAEIMALRQGGLVMQN
YRLIDATLYVTLEPCVMCAGAMIHSRIGRVVFGARDAKTGAAGSL
MDVLHHPGMNHRVEITEGILADECAALLSDFFRMRRQEIKAQKKA
QSSTDSGGSSGGSSGSETPGTSESATPESSGGSSGGSSEVEFSHE
YWMRHALTLAKRARDEREVPVGAVLVLNNRVIGEGWNRAIGLHDP
TAHAEIMALRQGGLVMQNYRLIDATLYVTFEPCVMCAGAMIHSRI
GRWFGVRNSKRGAAGSEMNVLNYPGMNHRVEITEGILADECAALL
CDFYRMPRQVFNAQKKAQSSINSGGSSGGSSGSETPGESESATPE
SSGGSSGGSDKKYSIGLAIGTNSVGWAVITDEYKVPSKKFKVLGN
TDRHSIKKNLIGALLFDSGETAEATRLKRTARRRYTRRKNRICYL
QEIFSNEMAKVDDSFFHRLEESFLVEEDKKHERHPIFGNIVDEVA
YHEKYPTIYHLRKKLVDSTDKADLRLIYLALAHMIKFRGHFLIEG
DLNPDNSDVDKLFIQLVQTYNQLFEENPINASGVDAKAILSARLS
KSRRLENLIAQLPGEKKNGLFGNLIALSLGLTPNFKSNFDLAEDA
KLQLSKDTYDDDLDNLLAQIGDQYADLFLAAKNLSDAILLSDILR
VNTEITKAPLSASMVKRYDEHHQDLTLLKALVRQQLPEKYKEIFF
DQSKNGYAGYIDGGASQEEFYKFIKPILEKMDGTEELLVKLNRED
LLRKQRTFDNGIIPHQIHLGELHAILRRQGDFYPFLKDNREKIEK
ILTFRIPYYVGPLARGNSRFAWMTRKSEETITPWNFEEVVDKGAS
AQSFIERMTNFDKNLPNEKVLPKHSLLYEYFTVYNELTKVKYVTE
GMRKPAFLSGEQKKAIVDLLFKTNRKVTVKQLKEDYFKKIECFDS
VEISGVEDRFNASLGTYHDLLKIIKDKDFLDNEENEDILEDIVLT
LTLFEDREMIEERLKTYAHLFDDKVMKQLKRLRYTGWGRLSRKLI
NGIRDKQSGKTILDFLKSDGFANRNFMQLIHDDSLTFKEDIQKAQ
VSCQGDSLHEHIANLAGSPAIKKGILQTVKWDELIKVMGGHKPEN
IVIEMARENQTTQKGQKNSRERMKRIEEGIKELGSQILKEHPVEN
TQLQNEKLYLYYLQNGRDMYVDQELDINRLSDYDVDHIVPQSFLK
DDSIENKVLTRSDKNRGKSDNVPSEEWKKMKNYWRQLLNAKLITQ
RKFDNLTKAERGGLSELDKAGFIKRQLAETRQITKHVAQILDSRM
NTKYDENDKLIREVKVITLKSKLVSDFRKDFQFYKVREINNYHHA
HDAYLNAWGTALIKKYPKLESEFVYGDYKVYDVRKMIAKSEQEIG
KATAKYFFYSNIMNFFKTEITLANGEIRKRPLIETNGETGEIVWD
KGRDFATVRKVLSMPQVNIVKKTEVQTGGFSKESILPKGNSDKLI
ARKKDWDPKKYGGFNSPTVAYSVLVVAKVEKGKSKKLKSVKELLG
ITIMERSSFEKNPIGFLEAKGYKEVKKDLIIKLPKYSLFELENGR
KRMLASASVLHKGNELALPSKYVNFLYLASHYEKLKGSSEDNKQK
QLFVEQHKHYLDEIIEQISEFSKRVILADANLDKVLSAYNKHRDK
PIREQAENIIHLFTLTNLGASAAFKYFDTTIGRKLYTSTKEVLDA
TLIHQSITGLYETRIDLSQLGGDSGGSKRTADGSEFEPKKKRKV
ABE8e-NRTH monomer editor: NLS, linker,
TadA*, SpCas9-NRTH
(SEQ ID NO: 3241)
MKRTADGSEFESPKKKRKVSEVEFSHEYWMRHALTLAKRARDERE
VPVGAVLVLNNRVIGEGWNRAIGLHDPTAHAEIMALRQGGLVMQN
YRLIDATLYVTFEPCVMCAGAMIHSRIGRVVFGVRNSKRGAAGSL
MNVLNYPGMNHRVEITEGILADECAALLCDFYRMPRQVFNAQKKA
QSSINSGGSSGGSSGSETPGTSESATPESSGGSSGGSDKKYSIGL
AIGTNSVGWAVITDEYKVPSKKFKVLGNTDRHSIKKNLIGALLFD
SGETAEATRLKRTARRRYTRRKNRICYLQEIFSNEMAKVDDSFFH
RLEESFLVEEDKKHERHPIFGNIVDEVAYHEKYPTIYHLRKKLVD
STDKADLRLIYLALAHMIKFRGHFLIEGDLNPDNSDVDKLFIQLV
QTYNQLFEENPINASGVDAKAILSARLSKSRRLENLIAQLPGEKK
NGLFGNLIALSLGLTPNFKSNFDLAEDAKLQLSKDTYDDDLDNLL
AQIGDQYADLFLAAKNLSDAILLSDILRVNTEITKAPLSASMVKR
YDEHHQDLTLLKALVRQQLPEKYKEIFFDQSKNGYAGYIDGGASQ
EEFYKFIKPILEKMDGTEELLVKLNREDLLRKQRTFDNGIIPHQI
HLGELHAILRRQGDFYPFLKDNREKIEKILTFRIPYYVGPLARGN
SRFAWMTRKSEETITPWNFEEVVDKGASAQSFIERMTNFDKNLPN
EKVLPKHSLLYEYFTVYNELTKVKYVTEGMRKPAFLSGEQKKAIV
DLLFKTNRKVTVKQLKEDYFKKIECFDSVEISGVEDRFNASLGTY
HDLLKIIKDKDFLDNEENEDILEDIVLTLTLFEDREMIEERLKTY
AHLFDDKVMKQLKRLRYTGWGRLSRKLINGIRDKQSGKTILDFLK
SDGFANRNFMQLIHDDSLTFKEDIQKAQVSCQGDSLHEHIANLAG
SPAIKKGILQTVKVVDELIKVMGGHKPENIVIEMARENQTTQKGQ
KNSRERMKRIEEGIKELGSQILKEHPVENTQLQNEKLYLYYLQNG
RDMYVDQELDINRLSDYDVDHIVPQSFLKDDSIENKVLTRSDKNR
GKSDNVPSEEVVKKMKNYWRQLLNAKLITQRKFDNLTKAERGGLS
ELDKAGFIKRQLAETRQITKHVAQILDSRMNTKYDENDKLIREVK
VITLKSKLVSDFRKDFQFYKVREINNYHHAHDAYLNAVVGTALIK
KYPKLESEFVYGDYKVYDVRKMIAKSEQEIGKATAKYFFYSNIMN
FFKTEITLANGEIRKRPLIETNGETGEIVWDKGRDFATVRKVLSM
PQVNIVKKTEVQTGGFSKESILPKGNSDKLIARKKDWDPKKYGGF
NSPTVAYSVLVVAKVEKGKSKKLKSVKELLGITIMERSSFEKNPI
GFLEAKGYKEVKKDLIIKLPKYSLFELENGRKRMLASASVLHKGN
ELALPSKYVNFLYLASHYEKLKGSSEDNKQKQLFVEQHKHYLDEI
IEQISEFSKRVILADANLDKVLSAYNKHRDKPIREQAENIIHLFT
LTNLGASAAFKYFDTTIGRKLYTSTKEVLDATLIHQSITGLYETR
IDLSQLGGD
SGGSKRTADGSEFEPKKKRKV
ABE8e-SpyMac dimer editor: NLS, wtTadA,
linker, TadA*, SpCas9-SpyMac
(SEQ ID NO: 3242)
MKRTADGSEFESPKKKRKVSEVEFSHEYWMRHALTLAKRAWDERE
VPVGAVLVHNNRVIGEGWNRPIGRHDPTAHAEIMALRQGGLVMQN
YRLIDATLYVTLEPCVMCAGAMIHSRIGRVVFGARDAKTGAAGSL
MDVLHHPGMNHRVEITEGILADECAALLSDFFRMRRQEIKAQKKA
QSSTDSGGSSGGSSGSETPGTSESATPESSGGSSGGSSEVEFSHE
YWMRHALTLAKRARDEREVPVGAVLVLNNRVIGEGWNRAIGLHDP
TAHAEIMALRQGGLVMQNYRLIDATLYVTFEPCVMCAGAMIHSRI
GRWFGVRNSKRGAAGSEMNVLNYPGMNHRVEITEGILADECAALL
CDFYRMPRQVFNAQKKAQSSINSGGSSGGSSGSETPGESESATPE
SSGGSSGGSDKKYSIGLAIGTNSVGWAVITDEYKVPSKKFKVLGN
TDRHSIKKNLIGALLFDSGETAEATRLKRTARRRYTRRKNRICYL
QEIFSNEMAKVDDSFFHRLEESFLVEEDKKHERHPIFGNIVDEVA
YHEKYPTIYHLRKKLVDSTDKADLRLIYLALAHMIKFRGHFLIEG
DLNPDNSDVDKLFIQLVQTYNQLFEENPINASGVDAKAILSARLS
KSRRLENLIAQLPGEKKNGLFGNLIALSLGLTPNFKSNFDLAEDA
KLQLSKDTYDDDLDNLLAQIGDQYADLFLAAKNLSDAILLSDILR
VNTEITKAPLSASMIKRYDEHHQDLTLLKALVRQQLPEKYKEIFF
DQSKNGYAGYIDGGASQEEFYKFIKPILEKMDGTEELLVKLNRED
LLRKQRTFDNGSIPHQIHLGELHAILRRQEDFYPFLKDNREKIEK
ILTFRIPYYVGPLARGNSRFAWMTRKSEETITPWNFEEVVDKGAS
AQSFIERMTNFDKNLPNEKVLPKHSLLYEYFTVYNELTKVKYVTE
GMRKPAFLSGEQKKAIVDLLFKTNRKVTVKQLKEDYFKKIECFDS
VEISGVEDRFNASLGTYHDLLKIIKDKDFLDNEENEDILEDIVLT
LTLFEDREMIEERLKTYAHLFDDKVMKQLKRRRYTGWGRLSRKLI
NGIRDKQSGKTILDFLKSDGFANRNFMQLIHDDSLTFKEDIQKAQ
VSGQGDSLHEHIANLAGSPAIKKGILQTVKWDELVKVMGRHKPEN
IVIEMARENQTTQKGQKNSRERMKRIEEGIKELGSQILKEHPVEN
TQLQNEKLYLYYLQNGRDMYVDQELDINRLSDYDVDHIVPQSFLK
DDSIDNKVLTRSDKNRGKSDNVPSEEWKKMKNYWRQLLNAKLITQ
RKFDNLTKAERGGLSELDKAGFIKRQLVETRQITKHVAQILDSRM
NTKYDENDKLIREVKVITLKSKLVSDFRKDFQFYKVREINNYHHA
HDAYLNAWGTALIKKYPKLESEFVYGDYKVYDVRKMIAKSEQEIG
KATAKYFFYSNIMNFFKTEITLANGEIRKRPLIETNGETGEIVWD
KGRDFATVRKVLSMPQVNIVKKTEIQTVGQNGGLFDDNPKSPLEV
TPSKLVPLKKELNPKKYGGYQKPTTAYPVLLITDTKQLIPISVMN
KKQFEQNPVKFLRDRGYQQVGKNDFIKLPKYTLVDIGDGIKRLWA
SSKEIHKGNQLWSKKSQILLYHAHHLDSDLSNDYLQNHNQQFDVL
FNEIISFSKKCKLGKEHIQKIENVYSNKKNSASIEELAESFIKLL
GFTQLGATSPFNFLGVKLNQKQYKGKKDYILPCTEGTLIRQSITG
LYETRVDLSKIGEDSGGSKETNDGSEEEEKKKRKV
ABE8e-SpyMac monomer editor: NLS, wtTadA,
linker, TadA*, SpCas9-SpyMac
(SEQ ID NO: 3243)
MKRTADGSEFESPKKKRKVSEVEFSHEYWMRHALTLAKRARDERE
VPVGAVLVLNNRVIGEGWNRAIGLHDPTAHAEIMALRQGGLVMQN
YRLIDATLYVTFEPCVMCAGAMIHSRIGRVVFGVRNSKRGAAGSL
MNVLNYPGMNHRVEITEGILADECAALLCDFYRMPRQVFNAQKKA
QSSINSGGSSGGSSGSETPGTSESATPESSGGSSGGSDKKYSIGL
AIGTNSVGWAVITDEYKVPSKKFKVLGNTDRHSIKKNLIGALLFD
SGETAEATRLKRTARRRYTRRKNRICYLQEIFSNEMAKVDDSFFH
RLEESFLVEEDKKHERHPIFGNIVDEVAYHEKYPTIYHLRKKLVD
STDKADLRLIYLALAHMIKFRGHFLIEGDLNPDNSDVDKLFIQLV
QTYNQLFEENPINASGVDAKAILSARLSKSRRLENLIAQLPGEKK
NGLFGNLIALSLGLTPNFKSNFDLAEDAKLQLSKDTYDDDLDNLL
AQIGDQYADLFLAAKNLSDAILLSDILRVNTEITKAPLSASMIKR
YDEHHQDLTLLKALVRQQLPEKYKEIFFDQSKNGYAGYIDGGASQ
EEFYKFIKPILEKMDGTEELLVKLNREDLLRKQRTFDNGSIPHQI
HLGELHAILRRQEDFYPFLKDNREKIEKILTFRIPYYVGPLARGN
SRFAWMTRKSEETITPWNFEEVVDKGASAQSFIERMTNFDKNLPN
EKVLPKHSLLYEYFTVYNELTKVKYVTEGMRKPAFLSGEQKKAIV
DLLFKTNRKVTVKQLKEDYFKKIECFDSVEISGVEDRFNASLGTY
HDLLKIIKDKDFLDNEENEDILEDIVLTLTLFEDREMIEERLKTY
AHLFDDKVMKQLKRRRYTGWGRLSRKLINGIRDKQSGKTILDFLK
SDGFANRNFMQLIHDDSLTFKEDIQKAQVSGQGDSLHEHIANLAG
SPAIKKGILQTVKVVDELVKVMGRHKPENIVIEMARENQTTQKGQ
KNSRERMKRIEEGIKELGSQILKEHPVENTQLQNEKLYLYYLQNG
RDMYVDQELDINRLSDYDVDHIVPQSFLKDDSIDNKVLTRSDKNR
GKSDNVPSEEVVKKMKNYWRQLLNAKLITQRKFDNLTKAERGGLS
ELDKAGFIKRQLVETRQITKHVAQILDSRMNTKYDENDKLIREVK
VITLKSKLVSDFRKDFQFYKVREINNYHHAHDAYLNAVVGTALIK
KYPKLESEFVYGDYKVYDVRKMIAKSEQEIGKATAKYFFYSNIMN
FFKTEITLANGEIRKRPLIETNGETGEIVWDKGRDFATVRKVLSM
PQVNIVKKTEIQTVGQNGGLFDDNPKSPLEVTPSKLVPLKKELNP
KKYGGYQKPTTAYPVLLITDTKQLIPISVMNKKQFEQNPVKFLRD
RGYQQVGKNDFIKLPKYTLVDIGDGIKRLWASSKEIHKGNQLVVS
KKSQILLYHAHHLDSDLSNDYLQNHNQQFDVLFNEIISFSKKCKL
GKEHIQKIENVYSNKKNSASIEELAESFIKLLGFTQLGATSPFNF
LGVKLNQKQYKGKKDYILPCTEGTLIRQSITGLYETRVDLSKIGE
DSGGSKRTADGSEFEPKKKRKV
ABE8e-VRQR-CP1041 dimer: NLS, wtTadA,
linker, TadA*, SpCas9-VRQR-
CP1041
(SEQ ID NO: 3244)
MKRTADGSEFESPKKKRKVSEVEFSHEYWMRHALTLAKRAWDERE
VPVGAVLVHNNRVIGEGWNRPIGRHDPTAHAEIMALRQGGLVMQN
YRLIDATLYVTLEPCVMCAGAMIHSRIGRVVFGARDAKTGAAGSL
MDVLHHPGMNHRVEITEGILADECAALLSDFFRMRRQEIKAQKKA
QSSTDSGGSSGGSSGSETPGTSESATPESSGGSSGGSSEVEFSHE
YWMRHALTLAKRARDEREVPVGAVLVLNNRVIGEGWNRAIGLHDP
TAHAEIMALRQGGLVMQNYRLIDATLYVTFEPCVMCAGAMIHSRI
GRWFGVRNSKRGAAGSEMNVLNYPGMNHRVEITEGILADECAALL
CDFYRMPRQVFNAQKKAQSSINSGGSSGGSSGSETPGTSESATPE
SSGGSSGGSNIMNFFKTEITLANGEIRKRPLIETNGETGEIVWDK
GRDFATVRKVLSMPQVNIVKKTEVQTGGFSKESIRPKRNSDKLIA
RKKDWDPKKYGGFVSPTVAYSVLVVAKVEKGKSKKLKSVKELLGI
TIMERSSFEKNPIDFLEAKGYKEVKKDLIIKLPKYSLFELENGRK
RMLASARFLQKGNELALPSKYVNFLYLASHYEKLKGSPEDNEQKQ
LFVEQHKHYLDEIIEQISEFSKRVILADANLDKVLSAYNKHRDKP
IREQAENIIHLFTLTNLGAPRAFKYFDTTIDRKVYRSTKEVLDAT
LIHQSITGLYETRIDLSQLGGDGGSGGSGGSGGSGGSGGSGGDKK
YSIGLAIGTNSVGWAVITDEYKVPSKKFKVLGNTDRHSIKKNLIG
ALLFDSGETAEATRLKRTARRRYTRRKNRICYLQEIFSNEMAKVD
DSFFHRLEESFLVEEDKKHERHPIFGNIVDEVAYHEKYPTIYHLR
KKLVDSTDKADLRLIYLALAHMIKFRGHFLIEGDLNPDNSDVDKL
FIQLVQTYNQLFEENPINASGVDAKAILSARLSKSRRLENLIAQL
PGEKKNGLFGNLIALSLGLTPNFKSNFDLAEDAKLQLSKDTYDDD
LDNLLAQIGDQYADLFLAAKNLSDAILLSDILRVNTEITKAPLSA
SMIKRYDEHHQDLTLLKALVRQQLPEKYKEIFFDQSKNGYAGYID
GGASQEEFYKFIKPILEKMDGTEELLVKLNREDLLRKQRTFDNGS
IPHQIHLGELHAILRRQEDFYPFLKDNREKIEKILTFRIPYYVGP
LARGNSRFAWMTRKSEETITPWNFEEVVDKGASAQSFIERMTNFD
KNLPNEKVLPKHSLLYEYFTVYNELTKVKYVTEGMRKPAFLSGEQ
KKAIVDLLFKTNRKVTVKQLKEDYFKKIECFDSVEISGVEDRFNA
SLGTYHDLLKIIKDKDFLDNEENEDILEDIVLTLTLFEDREMIEE
RLKTYAHLFDDKVMKQLKRRRYTGWGRLSRKLINGIRDKQSGKTI
LDFLKSDGFANRNFMQLIHDDSLTFKEDIQKAQVSGQGDSLHEHI
ANLAGSPAIKKGILQTVKWDELVKVMGRHKPENIVIEMARENQTT
QKGQKNSRERMKRIEEGIKELGSQILKEHPVENTQLQNEKLYLYY
LQNGRDMYVDQELDINRLSDYDVDHIVPQSFLKDDSIDNKVLTRS
DKNRGKSDNVPSEEWKKMKNYWRQLLNAKLITQRKFDNLTKAERG
GLSELDKAGFIKRQLVETRQITKHVAQILDSRMNTKYDENDKLIR
EVKVITLKSKLVSDFRKDFQFYKVREINNYHHAHDAYLNAWGTAL
IKKYPKLESEFVYGDYKVYDVRKMIAKSEQEIGKATAKYFFYSSG
GSKSRTADGSEFEPKKKRKV
ABE8e-VRQR-CP1041 monomer: NLS, linker,
TadA*, SpCas9-VRQR-CP1041
(SEQ ID NO: 3245)
MKRTADGSEFESPKKKRKVSEVEFSHEYWMRHALTLAKRARDERE
VPVGAVLVLNNRVIGEGWNRAIGLHDPTAHAEIMALRQGGLVMQN
YRLIDATLYVTFEPCVMCAGAMIHSRIGRVVFGVRNSKRGAAGSL
MNVLNYPGMNHRVEITEGILADECAALLCDFYRMPRQVFNAQKKA
QSSINSGGSSGGSSGSETPGTSESATPESSGGSSGGSNIMNFFKT
EITLANGEIRKRPLIETNGETGEIVWDKGRDFATVRKVLSMPQVN
IVKKTEVQTGGFSKESIRPKRNSDKLIARKKDWDPKKYGGFVSPT
VAYSVLVVAKVEKGKSKKLKSVKELLGITIMERSSFEKNPIDFLE
AKGYKEVKKDLIIKLPKYSLFELENGRKRMLASARFLQKGNELAL
PSKYVNFLYLASHYEKLKGSPEDNEQKQLFVEQHKHYLDEIIEQI
SEFSKRVILADANLDKVLSAYNKHRDKPIREQAENIIHLFTLTNL
GAPRAFKYFDTTIDRKVYRSTKEVLDATLIHQSITGLYETRIDLS
QLGGDGGSGGSGGSGGSGGSGGSGGDKKYSIGLAIGTNSVGWAVI
TDEYKVPSKKFKVLGNTDRHSIKKNLIGALLFDSGETAEATRLKR
TARRRYTRRKNRICYLQEIFSNEMAKVDDSFFHRLEESFLVEEDK
KHERHPIFGNIVDEVAYHEKYPTIYHLRKKLVDSTDKADLRLIYL
ALAHMIKFRGHFLIEGDLNPDNSDVDKLFIQLVQTYNQLFEENPI
NASGVDAKAILSARLSKSRRLENLIAQLPGEKKNGLFGNLIALSL
GLTPNFKSNFDLAEDAKLQLSKDTYDDDLDNLLAQIGDQYADLFL
AAKNLSDAILLSDILRVNTEITKAPLSASMIKRYDEHHQDLTLLK
ALVRQQLPEKYKEIFFDQSKNGYAGYIDGGASQEEFYKFIKPILE
KMDGTEELLVKLNREDLLRKQRTFDNGSIPHQIHLGELHAILRRQ
EDFYPFLKDNREKIEKILTFRIPYYVGPLARGNSRFAWMTRKSEE
TITPWNFEEVVDKGASAQSFIERMTNFDKNLPNEKVLPKHSLLYE
YFTVYNELTKVKYVTEGMRKPAFLSGEQKKAIVDLLFKTNRKVTV
KQLKEDYFKKIECFDSVEISGVEDRFNASLGTYHDLLKIIKDKDF
LDNEENEDILEDIVLTLTLFEDREMIEERLKTYAHLFDDKVMKQL
KRRRYTGWGRLSRKLINGIRDKQSGKTILDFLKSDGFANRNFMQL
IHDDSLTFKEDIQKAQVSGQGDSLHEHIANLAGSPAIKKGILQTV
KVVDELVKVMGRHKPENIVIEMARENQTTQKGQKNSRERMKRIEE
GIKELGSQILKEHPVENTQLQNEKLYLYYLQNGRDMYVDQELDIN
RLSDYDVDHIVPQSFLKDDSIDNKVLTRSDKNRGKSDNVPSEEVV
KKMKNYWRQLLNAKLITQRKFDNLTKAERGGLSELDKAGFIKRQL
VETRQITKHVAQILDSRMNTKYDENDKLIREVKVITLKSKLVSDF
RKDFQFYKVREINNYHHAHDAYLNAVVGTALIKKYPKLESEFVYG
DYKVYDVRKMIAKSEQEIGKATAKYFFYS
SGGSKRTADGSEFEPKKKRKV
ABE8e-SaCas9 dimer editor: NLS, wtTadA,
linker, TadA*, SaCas9
(SEQ ID NO: 205)
MKRTADGSEFESPKKKRKVSEVEFSHEYWMRHALTLAKRAWDERE
VPVGAVLVHNNRVIGEGWNRPIGRHDPTAHAEIMALRQGGLVMQN
YRLIDATLYVTLEPCVMCAGAMIHSRIGRVVFGARDAKTGAAGSL
MDVLHHPGMNHRVEITEGILADECAALLSDFFRMRRQEIKAQKKA
QSSTDSGGSSGGSSGSETPGTSESATPESSGGSSGGSSEVEFSHE
YWMRHALTLAKRARDEREVPVGAVLVLNNRVIGEGWNRAIGLHDP
TAHAEIMALRQGGLVMQNYRLIDATLYVTFEPCVMCAGAMIHSRI
GRWFGVRNSKRGAAGSEMNVLNYPGMNHRVEITEGILADECAALL
CDFYRMPRQVFNAQKKAQSSINSGGSSGGSSGSETPGTSESATPE
SSGGSSGGSGKRNYILGLAIGITSVGYGIIDYETRDVIDAGVRLF
KEANVENNEGRRSKRGARRLKRRRRHRIQRVKKLLFDYNLLTDHS
ELSGINPYEARVKGLSQKLSEEEFSAALLHLAKRRGVHNVNEVEE
DTGNELSTKEQISRNSKALEEKYVAELQLERLKKDGEVRGSINRF
KTSDYVKEAKQLLKVQKAYHQLDQSFIDTYIDLLETRRTYYEGPG
EGSPFGWKDIKEWYEMEMGHCTYFPEELRSVKYAYNADLYNALND
LNNLVITRDENEKLEYYEKFQIIENVFKQKKKPTLKQIAKEILVN
EEDIKGYRVTSTGKPEFTNLKVYHDIKDITARKEIIENAELLDQI
AKILTIYQSSEDIQEELTNLNSELTQEEIEQISNLKGYTGTHNLS
LKAINLILDELWHTNDNQIAIFNRLKLVPKKVDLSQQKEIPTTLV
DDFILSPWKRSFIQSIKVINAIIKKYGLPNDIIIELAREKNSKDA
QKMINEMQKRNRQTNERIEEIIRTTGKENAKYLIEKIKLHDMQEG
KCLYSLEAIPLEDLLNNPFNYEVDHIIPRSVSFDNSFNNKVLVKQ
EENSKKGNRTPFQYLSSSDSKISYETFKKHILNLAKGKGRISKTK
KEYLLEERDINRFSVQKDFINRNLVDTRYATRGEMNLLRSYFRVN
NLDVKVKSINGGFTSFLRRKWKFKKERNKGYKHHAEDALIIANAD
FIFKEWKKLDKAKKVMENQMFEEKQAESMPEIETEQEYKEIFITP
HQIKHIKDFKDYKYSHRVDKKPNRELINDTLYSTRKDDKGNTLIV
NNLNGLYDKDNDKLKKLINKSPEKLEMYHHDPQTYQKLKLIMEQY
GDEKNPLYKYYEETGNYLTKYSKKDNGPVIKKIKYYGNKLNAHLD
ITDDYPNSRNKWKLSLKPYRFDVYLDNGVYKFVTVKNLDVIKKEN
YYEVNSKCYEEAKKLKKISNQAEFIASFYNNDLIKINGELYRVIG
VNNDLLNRIEVNMIDITYREYLENMNDKRPPRIIKTIASKTQSIK
KYSTDILGNLYEVKSKKHPQIIKKGSGGSKRTADGSEFEPKKKRK
V
ABE8e-SaCas9 monomer editor: NLS, linker,
TadA*, SaCas9
(SEQ ID NO: 204)
MKRTADGSEFESPKKKRKVSEVEFSHEYWMRHALTLAKRARDERE
VPVGAVLVLNNRVIGEGWNRAIGLHDPTAHAEIMALRQGGLVMQN
YRLIDATLYVTFEPCVMCAGAMIHSRIGRVVFGVRNSKRGAAGSL
MNVLNYPGMNHRVEITEGILADECAALLCDFYRMPRQVFNAQKKA
QSSINSGGSSGGSSGSETPGTSESATPESSGGSSGGSGKRNYILG
LAIGITSVGYGIIDYETRDVIDAGVRLFKEANVENNEGRRSKRGA
RRLKRRRRHRIQRVKKLLFDYNLLTDHSELSGINPYEARVKGLSQ
KLSEEEFSAALLHLAKRRGVHNVNEVEEDTGNELSTKEQISRNSK
ALEEKYVAELQLERLKKDGEVRGSINRFKTSDYVKEAKQLLKVQK
AYHQLDQSFIDTYIDLLETRRTYYEGPGEGSPFGWKDIKEWYEML
MGHCTYFPEELRSVKYAYNADLYNALNDLNNLVITRDENEKLEYY
EKFQIIENVFKQKKKPTLKQIAKEILVNEEDIKGYRVTSTGKPEF
TNLKVYHDIKDITARKEIIENAELLDQIAKILTIYQSSEDIQEEL
TNLNSELTQEEIEQISNLKGYTGTHNLSLKAINLILDELWHTNDN
QIAIFNRLKLVPKKVDLSQQKEIPTTLVDDFILSPVVKRSFIQSI
KVINAIIKKYGLPNDIIIELAREKNSKDAQKMINEMQKRNRQTNE
RIEEIIRTTGKENAKYLIEKIKLHDMQEGKCLYSLEAIPLEDLLN
NPFNYEVDHIIPRSVSFDNSFNNKVLVKQEENSKKGNRTPFQYLS
SSDSKISYETFKKHILNLAKGKGRISKTKKEYLLEERDINRFSVQ
KDFINRNLVDTRYATRGLMNLLRSYFRVNNLDVKVKSINGGFTSF
LRRKWKFKKERNKGYKHHAEDALIIANADFIFKEWKKLDKAKKVM
ENQMFEEKQAESMPEIETEQEYKEIFITPHQIKHIKDFKDYKYSH
RVDKKPNRELINDTLYSTRKDDKGNTLIVNNLNGLYDKDNDKLKK
LINKSPEKLLMYHHDPQTYQKLKLIMEQYGDEKNPLYKYYEETGN
YLTKYSKKDNGPVIKKIKYYGNKLNAHLDITDDYPNSRNKVVKLS
LKPYRFDVYLDNGVYKFVTVKNLDVIKKENYYEVNSKCYEEAKKL
KKISNQAEFIASFYNNDLIKINGELYRVIGVNNDLLNRIEVNMID
ITYREYLENMNDKRPPRIIKTIASKTQSIKKYSTDILGNLYEVKS
KKHPQIIKK
GSGGSKRTADGSEFEPKKKRKV
ABESe-NRCH dimer editor: NLS, wtTadA,
linker, TadA*, SpCas9-NRCH
(SEQ ID NO: 3246)
MKRTADGSEFESPKKKRKVSEVEFSHEYWMRHALTLAKRAWDERE
VPVGAVLVHNNRVIGEGWNRPIGRHDPTAHAEIMALRQGGLVMQN
YRLIDATLYVTLEPCVMCAGAMIHSRIGRVVFGARDAKTGAAGSL
MDVLHHPGMNHRVEITEGILADECAALLSDFFRMRRQEIKAQKKA
QSSTDSGGSSGGSSGSETPGTSESATPESSGGSSGGSSEVEFSHE
YWMRHALTLAKRARDEREVPVGAVLVLNNRVIGEGWNRAIGLHDP
TAHAEIMALRQGGLVMQNYRLIDATLYVTFEPCVMCAGAMIHSRI
GRWFGVRNSKRGAAGSEMNVLNYPGMNHRVEITEGILADECAALL
CDFYRMPRQVFNAQKKAQSSINSGGSSGGSSGSETPGESESATPE
SSGGSSGGSDKKYSIGLAIGTNSVGWAVITDEYKVPSKKFKVLGN
TDRHSIKKNLIGALLFDSGETAEATRLKRTARRRYTRRKNRICYL
QEIFSNEMAKVDDSFFHRLEESFLVEEDKKHERHPIFGNIVDEVA
YHEKYPTIYHLRKKLVDSTDKADLRLIYLALAHMIKFRGHFLIEG
DLNPDNSDVDKLFIQLVQTYNQLFEENPINASGVDAKAILSARLS
KSRRLENLIAQLPGEKKNGLFGNLIALSLGLTPNFKSNFDLAEDA
KLQLSKDTYDDDLDNLLAQIGDQYADLFLAAKNLSDAILLSDILR
VNTEITKAPLSASMVKRYDEHHQDLTLLKALVRQQLPEKYKEIFF
DQSKNGYAGYIDGGASQEEFYKFIKPILEKMDGTEELLVKLNRED
LLRKQRTFDNGIIPHQIHLGELHAILRRQGDFYPFLKDNREKIEK
ILTFRIPYYVGPLARGNSRFAWMTRKSEETITPWNFEEVVDKGAS
AQSFIERMTNFDKNLPNEKVLPKHSLLYEYFTVYNELTKVKYVTE
GMRKPAFLSGEQKKAIVDLLFKTNRKVTVKQLKEDYFKKIECFDS
VEISGVEDRFNASLGTYHDLLKIIKDKDFLDNEENEDILEDIVLT
LTLFEDREMIEERLKTYAHLFDDKVMKQLKRLRYTGWGRLSRKLI
NGIRDKQSGKTILDFLKSDGFANRNFMQLIHDDSLTFKEDIQKAQ
VSCQGDSLHEHIANLAGSPAIKKGILQTVKWDELIKVMGGHKPEN
IVIEMARENQTTQKGQKNSRERMKRIEEGIKELGSQILKEHPVEN
TQLQNEKLYLYYLQNGRDMYVDQELDINRLSDYDVDHIVPQSFLK
DDSIENKVLTRSDKNRGKSDNVPSEEWKKMKNYWRQLLNAKLITQ
RKFDNLTKAERGGLSELDKAGFIKRQLAETRQITKHVAQILDSRM
NTKYDENDKLIREVKVITLKSKLVSDFRKDFQFYKVREINNYHHA
HDAYLNAWGTALIKKYPKLESEFVYGDYKVYDVRKMIAKSEQEIG
KATAKYFFYSNIMNFFKTEITLANGEIRKRPLIETNGETGEIVWD
KGRDFATVRKVLSMPQVNIVKKTEVQTGGFSKESILPKGNSDKLI
ARKKDWDPKKYGGFNSPTVAYSVLVVAKVEKGKSKKLKSVKELLG
ITIMERSSFEKNPIDFLEAKGYKEVKKDLIIKLPKYSLFELENGR
KRMLASAGVLQKGNELALPSKYVNFLYLASHYEKLKGSPEDNEQK
QLFVEQHKHYLDEIIEQISEFSKRVILADANLDKVLSAYNKHRDK
PIREQAENIIHLFTLTNLGAPAAFKYFDTTINRKQYNTTKEVLDA
TLIRQSITGLYETRIDLSQLGGDSGGSKRTADGSYYYYKKKRKN
ABEZe-NRCH monomer editor: NLS, linker,
TadA*, SpCas9-NRCH
(SEQ ID NO: 3247)
MKRTADGSEFESPKKKRKVSEVEFSHEYWMRHALTLAKRARDERE
VPVGAVLVLNNRVIGEGWNRAIGLHDPTAHAEIMALRQGGLVMQN
YRLIDATLYVTFEPCVMCAGAMIHSRIGRVVFGVRNSKRGAAGSL
MNVLNYPGMNHRVEITEGILADECAALLCDFYRMPRQVFNAQKKA
QSSINSGGSSGGSSGSETPGTSESATPESSGGSSGGSDKKYSIGL
AIGTNSVGWAVITDEYKVPSKKFKVLGNTDRHSIKKNLIGALLFD
SGETAEATRLKRTARRRYTRRKNRICYLQEIFSNEMAKVDDSFFH
RLEESFLVEEDKKHERHPIFGNIVDEVAYHEKYPTIYHLRKKLVD
STDKADLRLIYLALAHMIKFRGHFLIEGDLNPDNSDVDKLFIQLV
QTYNQLFEENPINASGVDAKAILSARLSKSRRLENLIAQLPGEKK
NGLFGNLIALSLGLTPNFKSNFDLAEDAKLQLSKDTYDDDLDNLL
AQIGDQYADLFLAAKNLSDAILLSDILRVNTEITKAPLSASMVKR
YDEHHQDLTLLKALVRQQLPEKYKEIFFDQSKNGYAGYIDGGASQ
EEFYKFIKPILEKMDGTEELLVKLNREDLLRKQRTFDNGIIPHQI
HLGELHAILRRQGDFYPFLKDNREKIEKILTFRIPYYVGPLARGN
SRFAWMTRKSEETITPWNFEEVVDKGASAQSFIERMTNFDKNLPN
EKVLPKHSLLYEYFTVYNELTKVKYVTEGMRKPAFLSGEQKKAIV
DLLFKTNRKVTVKQLKEDYFKKIECFDSVEISGVEDRFNASLGTY
HDLLKIIKDKDFLDNEENEDILEDIVLTLTLFEDREMIEERLKTY
AHLFDDKVMKQLKRLRYTGWGRLSRKLINGIRDKQSGKTILDFLK
SDGFANRNFMQLIHDDSLTFKEDIQKAQVSCQGDSLHEHIANLAG
SPAIKKGILQTVKVVDELIKVMGGHKPENIVIEMARENQTTQKGQ
KNSRERMKRIEEGIKELGSQILKEHPVENTQLQNEKLYLYYLQNG
RDMYVDQELDINRLSDYDVDHIVPQSFLKDDSIENKVLTRSDKNR
GKSDNVPSEEVVKKMKNYWRQLLNAKLITQRKFDNLTKAERGGLS
ELDKAGFIKRQLAETRQITKHVAQILDSRMNTKYDENDKLIREVK
VITLKSKLVSDFRKDFQFYKVREINNYHHAHDAYLNAVVGTALIK
KYPKLESEFVYGDYKVYDVRKMIAKSEQEIGKATAKYFFYSNIMN
FFKTEITLANGEIRKRPLIETNGETGEIVWDKGRDFATVRKVLSM
PQVNIVKKTEVQTGGFSKESILPKGNSDKLIARKKDWDPKKYGGF
NSPTVAYSVLVVAKVEKGKSKKLKSVKELLGITIMERSSFEKNPI
DFLEAKGYKEVKKDLIIKLPKYSLFELENGRKRMLASAGVLQKGN
ELALPSKYVNFLYLASHYEKLKGSPEDNEQKQLFVEQHKHYLDEI
IEQISEFSKRVILADANLDKVLSAYNKHRDKPIREQAENIIHLFT
LTNLGAPAAFKYFDTTINRKQYNTTKEVLDATLIRQSITGLYETR
IDLSQLGGD
SGGSKRTADGSEFEPKKKRKV
ABE8e-NRRHdimer editor: NLS, wtTadA, linker,
TadA*, SpCas9-NRRH
(SEQ ID NO: 3248)
MKRTADGSEFESPKKKRKVSEVEFSHEYWMRHALTLAKRAWDERE
VPVGAVLVHNNRVIGEGWNRPIGRHDPTAHAEIMALRQGGLVMQN
YRLIDATLYVTLEPCVMCAGAMIHSRIGRVVFGARDAKTGAAGSL
MDVLHHPGMNHRVEITEGILADECAALLSDFFRMRRQEIKAQKKA
QSSTDSGGSSGGSSGSETPGTSESATPESSGGSSGGSSEVEFSHE
YWMRHALTLAKRARDEREVPVGAVLVLNNRVIGEGWNRAIGLHDP
TAHAEIMALRQGGLVMQNYRLIDATLYVTFEPCVMCAGAMIHSRI
GRWFGVRNSKRGAAGSEMNVLNYPGMNHRVEITEGILADECAALL
CDFYRMPRQVFNAQKKAQSSINSGGSSGGSSGSETPGTSESATPE
SSGGSSGGSDKKYSIGLAIGTNSVGWAVITDEYKVPSKKFKVLGN
TDRHSIKKNLIGALLFDSGETAEATRLKRTARRRYTRRKNRICYL
QEIFSNEMAKVDDSFFHRLEESFLVEEDKKHERHPIFGNIVDEVA
YHEKYPTIYHLRKKLVDSTDKADLRLIYLALAHMIKFRGHFLIEG
DLNPDNSDVDKLFIQLVQTYNQLFEENPINASGVDAKAILSARLS
KSRRLENLIAQLPGEKKNGLFGNLIALSLGLTPNFKSNFDLAEDA
KLQLSKDTYDDDLDNLLAQIGDQYADLFLAAKNLSDAILLSDILR
VNTEITKAPLSASMVKRYDEHHQDLTLLKALVRQQLPEKYKEIFF
DQSKNGYAGYIDGGASQEEFYKFIKPILEKMDGTEELLVKLNRED
LLRKQRTFDNGIIPHQIHLGELHAILRRQGDFYPFLKDNREKIEK
ILTFRIPYYVGPLARGNSRFAWMTRKSEETITPWNFEEVVDKGAS
AQSFIERMTNFDKNLPNEKVLPKHSLLYEYFTVYNELTKVKYVTE
GMRKPAFLSGEQKKAIVDLLFKTNRKVTVKQLKEDYFKKIECFDS
VEISGVEDRFNASLGTYHDLLKIIKDKDFLDNEENEDILEDIVLT
LTLFEDREMIEERLKTYAHLFDDKVMKQLKRLRYTGWGRLSRKLI
NGIRDKQSGKTILDFLKSDGFANRNFMQLIHDDSLTFKEDIQKAQ
VSCQGDSLHEHIANLAGSPAIKKGILQTVKWDELIKVMGGHKPEN
IVIEMARENQTTQKGQKNSRERMKRIEEGIKELGSQILKEHPVEN
TQLQNEKLYLYYLQNGRDMYVDQELDINRLSDYDVDHIVPQSFLK
DDSIENKVLTRSDKNRGKSDNVPSEEWKKMKNYWRQLLNAKLITQ
RKFDNLTKAERGGLSELDKAGFIKRQLAETRQITKHVAQILDSRM
NTKYDENDKLIREVKVITLKSKLVSDFRKDFQFYKVREINNYHHA
HDAYLNAWGTALIKKYPKLESEFVYGDYKVYDVRKMIAKSEQEIG
KATAKYFFYSNIMNFFKTEITLANGEIRKRPLIETNGETGEIVWD
KGRDFATVRKVLSMPQVNIVKKTEVQTGGFSKESILPKGNSDKLI
ARKKDWDPKKYGGFNSPTAAYSVLVVAKVEKGKSKKLKSVKELLG
ITIMERSSFEKNPIGFLEAKGYKEVKKDLIIKLPKYSLFELENGR
KRMLASAGVLHKGNELALPSKYVNFLYLASHYEKLKGSPEDNEQK
QLFVEQHKHYLDEIIEQISEFSKRVILADANLDKVLSAYNKHRDK
PIREQAENIIHLFTLTNLGVPAAFKYFDTTIDKKRYTSTKEVLDA
TLIHQSITGLYETRIDLSQLGGDSGGSKWYNDGSYYPPKKKRKN
ABE8e-NRRH monomer editor: NLS, linker,
TadA*, SpCas9-NRRH
(SEQ ID NO: 3249)
MKRTADGSEFESPKKKRKVSEVEFSHEYWMRHALTLAKRARDERE
VPVGAVLVLNNRVIGEGWNRAIGLHDPTAHAEIMALRQGGLVMQN
YRLIDATLYVTFEPCVMCAGAMIHSRIGRVVFGVRNSKRGAAGSL
MNVLNYPGMNHRVEITEGILADECAALLCDFYRMPRQVFNAQKKA
QSSINSGGSSGGSSGSETPGTSESATPESSGGSSGGSDKKYSIGL
AIGTNSVGWAVITDEYKVPSKKFKVLGNTDRHSIKKNLIGALLFD
SGETAEATRLKRTARRRYTRRKNRICYLQEIFSNEMAKVDDSFFH
RLEESFLVEEDKKHERHPIFGNIVDEVAYHEKYPTIYHLRKKLVD
STDKADLRLIYLALAHMIKFRGHFLIEGDLNPDNSDVDKLFIQLV
QTYNQLFEENPINASGVDAKAILSARLSKSRRLENLIAQLPGEKK
NGLFGNLIALSLGLTPNFKSNFDLAEDAKLQLSKDTYDDDLDNLL
AQIGDQYADLFLAAKNLSDAILLSDILRVNTEITKAPLSASMVKR
YDEHHQDLTLLKALVRQQLPEKYKEIFFDQSKNGYAGYIDGGASQ
EEFYKFIKPILEKMDGTEELLVKLNREDLLRKQRTFDNGIIPHQI
HLGELHAILRRQGDFYPFLKDNREKIEKILTFRIPYYVGPLARGN
SRFAWMTRKSEETITPWNFEEVVDKGASAQSFIERMTNFDKNLPN
EKVLPKHSLLYEYFTVYNELTKVKYVTEGMRKPAFLSGEQKKAIV
DLLFKTNRKVTVKQLKEDYFKKIECFDSVEISGVEDRFNASLGTY
HDLLKIIKDKDFLDNEENEDILEDIVLTLTLFEDREMIEERLKTY
AHLFDDKVMKQLKRLRYTGWGRLSRKLINGIRDKQSGKTILDFLK
SDGFANRNFMQLIHDDSLTFKEDIQKAQVSCQGDSLHEHIANLAG
SPAIKKGILQTVKVVDELIKVMGGHKPENIVIEMARENQTTQKGQ
KNSRERMKRIEEGIKELGSQILKEHPVENTQLQNEKLYLYYLQNG
RDMYVDQELDINRLSDYDVDHIVPQSFLKDDSIENKVLTRSDKNR
GKSDNVPSEEVVKKMKNYWRQLLNAKLITQRKFDNLTKAERGGLS
ELDKAGFIKRQLAETRQITKHVAQILDSRMNTKYDENDKLIREVK
VITLKSKLVSDFRKDFQFYKVREINNYHHAHDAYLNAVVGTALIK
KYPKLESEFVYGDYKVYDVRKMIAKSEQEIGKATAKYFFYSNIMN
FFKTEITLANGEIRKRPLIETNGETGEIVWDKGRDFATVRKVLSM
PQVNIVKKTEVQTGGFSKESILPKGNSDKLIARKKDWDPKKYGGF
NSPTAAYSVLVVAKVEKGKSKKLKSVKELLGITIMERSSFEKNPI
GFLEAKGYKEVKKDLIIKLPKYSLFELENGRKRMLASAGVLHKGN
ELALPSKYVNFLYLASHYEKLKGSPEDNEQKQLFVEQHKHYLDEI
IEQISEFSKRVILADANLDKVLSAYNKHRDKPIREQAENIIHLFT
LTNLGVPAAFKYFDTTIDKKRYTSTKEVLDATLIHQSITGLYETR
IDLSQLGGDSGGSKRTADGSEFEPKKKRKV
SaKKH-ABE8e dimer editor: NLS, wtTadA,
linker, TadA*, SaKKH
(SEQ ID NO: 3250)
MKRTADGSEFESPKKKRKVSEVEFSHEYWMRHALTLAKRAWDERE
VPVGAVLVHNNRVIGEGWNRPIGRHDPTAHAEIMALRQGGLVMQN
YRLIDATLYVTLEPCVMCAGAMIHSRIGRVVFGARDAKTGAAGSL
MDVLHHPGMNHRVEITEGILADECAALLSDFFRMRRQEIKAQKKA
QSSTDSGGSSGGSSGSETPGTSESATPESSGGSSGGSSEVEFSHE
YWMRHALTLAKRARDEREVPVGAVLVLNNRVIGEGWNRAIGLHDP
TAHAEIMALRQGGLVMQNYRLIDATLYVTFEPCVMCAGAMIHSRI
GRVVFGVRNSKRGAAGSEMNVLNYPGMNHRVEITEGILADECAAL
LCDFYRMPRQVFNAQKKAQSSINSGGSSGGSSGSETPGTSESATP
ESSGGSSGGSKRNYILGLAIGITSVGYGIIDYETRDVIDAGV
RLFKEANVENNEGRRSKRGARRLKRRRRHRIQRVKKLLFDYNLLT
DHSELSGINPYEARVKGLSQKLSEEEFSAALLHLAKRRGVHNVNE
VEEDTGNELSTKEQISRNSKALEEKYVAELQLERLKKDGEVRGSI
NRFKTSDYVKEAKQLLKVQKAYHQLDQSFIDTYIDLLETRRTYYE
GPGEGSPFGWKDIKEWYEMEMGHCTYFPEELRSVKYAYNADLYNA
LNDLNNLVITRDENEKLEYYEKFQIIENVFKQKKKPTLKQIAKEI
LVNEEDIKGYRVTSTGKPEFTNLKVYHDIKDITARKEIIENAELL
DQIAKILTIYQSSEDIQEELTNLNSELTQEEIEQISNLKGYTGTH
NLSLKAINLILDELWHTNDNQIAIFNRLKLVPKKVDLSQQKEIPT
TLVDDFILSPWKRSFIQSIKVINAIIKKYGLPNDIIIELAREKNS
KDAQKMINEMQKRNRQTNERIEEIIRTTGKENAKYLIEKIKLHDM
QEGKCLYSLEAIPLEDLLNNPFNYEVDHIIPRSVSFDNSFNNKVL
VKQEENSKKGNRTPFQYLSSSDSKISYETFKKHILNLAKGKGRIS
KTKKEYLLEERDINRFSVQKDFINRNLVDTRYATRGEMNLLRSYF
RVNNLDVKVKSINGGFTSFLRRKWKFKKERNKGYKHHAEDALIIA
NADFIFKEWKKLDKAKKVMENQMFEEKQAESMPEIETEQEYKEIF
ITPHQIKHIKDFKDYKYSHRVDKKPNRKLINDTLYSTRKDDKGNT
LIVNNLNGLYDKDNDKLKKLINKSPEKLLMYHHDPQTYQKLKLIM
EQYGDEKNPLYKYYEETGNYLTKYSKKDNGPVIKKIKYYGNKLNA
HLDITDDYPNSRNKWKLSLKPYRFDVYLDNGVYKFVTVKNLDVIK
KENYYEVNSKCYEEAKKLKKISNQAEFIASFYKNDLIKINGELYR
VIGVNNDLLNRIEVNMIDITYREYLENMNDKRPPHIIKTIASKTQ
SIKKYSTDILGNLYEVKSKKHPQIIKKGSGGSKRTADGSEFEPKK
KRKV
SaKKH-ABESe monomer editor: NLS, linker,
TadA*, SaKKH
(SEQ ID NO: 3251)
MKRTADGSEFESPKKKRKVSEVEFSHEYWMRHALTLAKRARDERE
VPVGAVLVLNNRVIGEGWNRAIGLHDPTAHAEIMALRQGGLVMQN
YRLIDATLYVTFEPCVMCAGAMIHSRIGRVVFGVRNSKRGAAGSL
MNVLNYPGMNHRVEITEGILADECAALLCDFYRMPRQVFNAQKKA
QSSINSGGSSGGSSGSETPGTSESATPESSGGSSGGSGKRNYILG
LAIGITSVGYGIIDYETRDVIDAGVRLFKEANVENNEGRRSKRGA
RRLKRRRRHRIQRVKKLLFDYNLLTDHSELSGINPYEARVKGLSQ
KLSEEEFSAALLHLAKRRGVHNVNEVEEDTGNELSTKEQISRNSK
ALEEKYVAELQLERLKKDGEVRGSINRFKTSDYVKEAKQLLKVQK
AYHQLDQSFIDTYIDLLETRRTYYEGPGEGSPFGWKDIKEWYEML
MGHCTYFPEELRSVKYAYNADLYNALNDLNNLVITRDENEKLEYY
EKFQIIENVFKQKKKPTLKQIAKEILVNEEDIKGYRVTSTGKPEF
TNLKVYHDIKDITARKEIIENAELLDQIAKILTIYQSSEDIQEEL
TNLNSELTQEEIEQISNLKGYTGTHNLSLKAINLILDELWHTNDN
QIAIFNRLKLVPKKVDLSQQKEIPTTLVDDFILSPVVKRSFIQSI
KVINAIIKKYGLPNDIIIELAREKNSKDAQKMINEMQKRNRQTNE
RIEEIIRTTGKENAKYLIEKIKLHDMQEGKCLYSLEAIPLEDLLN
NPFNYEVDHIIPRSVSFDNSFNNKVLVKQEENSKKGNRTPFQYLS
SSDSKISYETFKKHILNLAKGKGRISKTKKEYLLEERDINRFSVQ
KDFINRNLVDTRYATRGLMNLLRSYFRVNNLDVKVKSINGGFTSF
LRRKWKFKKERNKGYKHHAEDALIIANADFIFKEWKKLDKAKKVM
ENQMFEEKQAESMPEIETEQEYKEIFITPHQIKHIKDFKDYKYSH
RVDKKPNRKLINDTLYSTRKDDKGNTLIVNNLNGLYDKDNDKLKK
LINKSPEKLLMYHHDPQTYQKLKLIMEQYGDEKNPLYKYYEETGN
YLTKYSKKDNGPVIKKIKYYGNKLNAHLDITDDYPNSRNKVVKLS
LKPYRFDVYLDNGVYKFVTVKNLDVIKKENYYEVNSKCYEEAKKL
KKISNQAEFIASFYKNDLIKINGELYRVIGVNNDLLNRIEVNMID
ITYREYLENMNDKRPPHIIKTIASKTQSIKKYSTDILGNLYEVKS
KKHPQIIKKG
SGGSKRTADGSEFEPKKKRKV
ABESe-NG dimer editor: NLS, wtTadA, linker,
TadA*, SpCas9-NG
(SEQ ID NO: 213)
MKRTADGSEFESPKKKRKVSEVEFSHEYWMRHALTLAKRAWDERE
VPVGAVLVHNNRVIGEGWNRPIGRHDPTAHAEIMALRQGGLVMQN
YRLIDATLYVTLEPCVMCAGAMIHSRIGRVVFGARDAKTGAAGSL
MDVLHHPGMNHRVEITEGILADECAALLSDFFRMRRQEIKAQKKA
QSSTDSGGSSGGSSGSETPGTSESATPESSGGSSGGSSEVEFSHE
YWMRHALTLAKRARDEREVPVGAVLVLNNRVIGEGWNRAIGLHDP
TAHAEIMALRQGGLVMQNYRLIDATLYVTFEPCVMCAGAMIHSRI
GRVVFGVRNSKRGAAGSEMNVLNYPGMNHRVEITEGILADECAAL
LCDFYRMPRQVFNAQKKAQSSINSGGSSGGSSGSETPGTSESATP
ESSGGSSGGSDKKYSIGLAIGTNSVGWAVITDEYKVPSKKFKVLG
NTDRHSIKKNLIGALLFDSGETAEATRLKRTARRRYTRRKNRICY
LQEIFSNEMAKVDDSFFHRLEESFLVEEDKKHERHPIFGNIVDEV
AYHEKYPTIYHLRKKLVDSTDKADLRLIYLALAHMIKFRGHFLIE
GDLNPDNSDVDKLFIQLVQTYNQLFEENPINASGVDAKAILSARL
SKSRRLENLIAQLPGEKKNGLFGNLIALSLGLTPNFKSNFDLAED
AKLQLSKDTYDDDLDNLLAQIGDQYADLFLAAKNLSDAILLSDIL
RVNTEITKAPLSASMIKRYDEHHQDLTLLKALVRQQLPEKYKEIF
FDQSKNGYAGYIDGGASQEEFYKFIKPILEKMDGTEELLVKLNRE
DLLRKQRTFDNGSIPHQIHLGELHAILRRQEDFYPFLKDNREKIE
KILTFRIPYYVGPLARGNSRFAWMTRKSEETITPWNFEEVVDKGA
SAQSFIERMTNFDKNLPNEKVLPKHSLLYEYFTVYNELTKVKYVT
EGMRKPAFLSGEQKKAIVDLLFKTNRKVTVKQLKEDYFKKIECFD
SVEISGVEDRFNASLGTYHDLLKIIKDKDFLDNEENEDILEDIVL
TLTLFEDREMIEERLKTYAHLFDDKVMKQLKRRRYTGWGRLSRKL
INGIRDKQSGKTILDFLKSDGFANRNFMQLIHDDSLTFKEDIQKA
QVSGQGDSLHEHIANLAGSPAIKKGILQTVKWDELVKVMGRHKPE
NIVIEMARENQTTQKGQKNSRERMKRIEEGIKELGSQILKEHPVE
NTQLQNEKLYLYYLQNGRDMYVDQELDINRLSDYDVDHIVPQSFL
KDDSIDNKVLTRSDKNRGKSDNVPSEEWKKMKNYWRQLLNAKLIT
QRKFDNLTKAERGGLSELDKAGFIKRQLVETRQITKHVAQILDSR
MNTKYDENDKLIREVKVITLKSKLVSDFRKDFQFYKVREINNYHH
AHDAYLNAWGTALIKKYPKLESEFVYGDYKVYDVRKMIAKSEQEI
GKATAKYFFYSNIMNFFKTEITLANGEIRKRPLIETNGETGEIVW
DKGRDFATVRKVLSMPQVNIVKKTEVQTGGFSKESIRPKRNSDKL
IARKKDWDPKKYGGFVSPTVAYSVLWAKVEKGKSKKLKSVKELLG
ITIMERSSFEKNPIDFLEAKGYKEVKKDLIIKLPKYSLFELENGR
KRMLASARFLQKGNELALPSKYVNFLYLASHYEKLKGSPEDNEQK
QLFVEQHKHYLDEIIEQISEFSKRVILADANLDKVLSAYNKHRDK
PIREQAENIIHLFTLTNLGAPRAFKYFDTTIDRKVYRSTKEVLDA
TLIHQSITGLYETRIDLSQLGGDSGGSKRTADGSEFEPKKKRKV
ABEZe-NG monomer editor: NLS, linker,
TadA*, SpCas9-NG (“NG-ABEZe”)
MKRTADGSEFESPKKKRKVSEVEFSHEYWMRHALTLAKRARDERE
(SEQ ID NO: 212)
VPVGAVLVLNNRVIGEGWNRAIGLHDPTAHAEIMALRQGGLVMQN
YRLIDATLYVTFEPCVMCAGAMIHSRIGRVVFGVRNSKRGAAGSL
MNVLNYPGMNHRVEITEGILADECAALLCDFYRMPRQVFNAQKKA
QSSINSGGSSGGSSGSETPGTSESATPESSGGSSGGSDKKYSIGL
AIGTNSVGWAVITDEYKVPSKKFKVLGNTDRHSIKKNLIGALLFD
SGETAEATRLKRTARRRYTRRKNRICYLQEIFSNEMAKVDDSFFH
RLEESFLVEEDKKHERHPIFGNIVDEVAYHEKYPTIYHLRKKLVD
STDKADLRLIYLALAHMIKFRGHFLIEGDLNPDNSDVDKLFIQLV
QTYNQLFEENPINASGVDAKAILSARLSKSRRLENLIAQLPGEKK
NGLFGNLIALSLGLTPNFKSNFDLAEDAKLQLSKDTYDDDLDNLL
AQIGDQYADLFLAAKNLSDAILLSDILRVNTEITKAPLSASMIKR
YDEHHQDLTLLKALVRQQLPEKYKEIFFDQSKNGYAGYIDGGASQ
EEFYKFIKPILEKMDGTEELLVKLNREDLLRKQRTFDNGSIPHQI
HLGELHAILRRQEDFYPFLKDNREKIEKILTFRIPYYVGPLARGN
SRFAWMTRKSEETITPWNFEEVVDKGASAQSFIERMTNFDKNLPN
EKVLPKHSLLYEYFTVYNELTKVKYVTEGMRKPAFLSGEQKKAIV
DLLFKTNRKVTVKQLKEDYFKKIECFDSVEISGVEDRFNASLGTY
HDLLKIIKDKDFLDNEENEDILEDIVLTLTLFEDREMIEERLKTY
AHLFDDKVMKQLKRRRYTGWGRLSRKLINGIRDKQSGKTILDFLK
SDGFANRNFMQLIHDDSLTFKEDIQKAQVSGQGDSLHEHIANLAG
SPAIKKGILQTVKVVDELVKVMGRHKPENIVIEMARENQTTQKGQ
KNSRERMKRIEEGIKELGSQILKEHPVENTQLQNEKLYLYYLQNG
RDMYVDQELDINRLSDYDVDHIVPQSFLKDDSIDNKVLTRSDKNR
GKSDNVPSEEVVKKMKNYWRQLLNAKLITQRKFDNLTKAERGGLS
ELDKAGFIKRQLVETRQITKHVAQILDSRMNTKYDENDKLIREVK
VITLKSKLVSDFRKDFQFYKVREINNYHHAHDAYLNAVVGTALIK
KYPKLESEFVYGDYKVYDVRKMIAKSEQEIGKATAKYFFYSNIMN
FFKTEITLANGEIRKRPLIETNGETGEIVWDKGRDFATVRKVLSM
PQVNIVKKTEVQTGGFSKESIRPKRNSDKLIARKKDWDPKKYGGF
VSPTVAYSVLVVAKVEKGKSKKLKSVKELLGITIMERSSFEKNPI
DFLEAKGYKEVKKDLIIKLPKYSLFELENGRKRMLASARFLQKGN
ELALPSKYVNFLYLASHYEKLKGSPEDNEQKQLFVEQHKHYLDEI
IEQISEFSKRVILADANLDKVLSAYNKHRDKPIREQAENIIHLFT
LTNLGAPRAFKYFDTTIDRKVYRSTKEVLDATLIHQSITGLYETR
IDLSQLGGD
SGGSKRTADGSEFEPKKKRKV
ABE8e-CP 1041 dimer editor: NLS, wtTadA,
linker, TadA*, CP 1041
(SEQ ID NO: 3252)
MKRTADGSEFESPKKKRKVSEVEFSHEYWMRHALTLAKRAWDERE
VPVGAVLVHNNRVIGEGWNRPIGRHDPTAHAEIMALRQGGLVMQN
YRLIDATLYVTLEPCVMCAGAMIHSRIGRVVFGARDAKTGAAGSL
MDVLHHPGMNHRVEITEGILADECAALLSDFFRMRRQEIKAQKKA
QSSTDSGGSSGGSSGSETPGTSESATPESSGGSSGGSSEVEFSHE
YWMRHALTLAKRARDEREVPVGAVLVLNNRVIGEGWNRAIGLHDP
TAHAEIMALRQGGLVMQNYRLIDATLYVTFEPCVMCAGAMIHSRI
GRVVFGVRNSKRGAAGSEMNVLNYPGMNHRVEITEGILADECAAL
LCDFYRMPRQVFNAQKKAQSSINSGGSSGGSSGSYYYGYSPSKYY
PSSGGSSGGSNIMNFFKTEITLANGEIRKRPLIETNGETGEIVWD
KGRDFATVRKVLSMPQVNIVKKTEVQTGGFSKESILPKRNSDKLI
ARKKDWDPKKYGGFDSPTVAYSVLVVAKVEKGKSKKLKSVKELLG
ITIMERSSFEKNPIDFLEAKGYKEVKKDLIIKLPKYSLFELENGR
KRMLASAGELQKGNELALPSKYVNFLYLASHYEKLKGSPEDNEQK
QLFVEQHKHYLDEIIEQISEFSKRVILADANLDKVLSAYNKHRDK
PIREQAENIIHLFTLTNLGAPAAFKYFDTTIDRKRYTSTKEVLDA
TLIHQSITGLYETRIDLSQLGGDGGSGGSGGSGGSGGSGGSGGDK
KYSIGLAIGTNSVGWAVITDEYKVPSKKFKVLGNTDRHSIKKNLI
GALLFDSGETAEATRLKRTARRRYTRRKNRJCYLQEIFSNEMAKV
DDSFFHRLEESFLVEEDKKHERHPIFGNIVDEVAYHEKYPTIYHL
RKKLVDSTDKADLRLIYLALAHMIKFRGHFLIEGDLNPDNSDVDK
LFIQLVQTYNQLFEENPINASGVDAKAILSARLSKSRRLENLIAQ
LPGEKKNGLFGNLIALSLGLTPNFKSNFDLAEDAKLQLSKDTYDD
DLDNLLAQIGDQYADLFLAAKNLSDAILLSDILRVNTEITKAPLS
ASMIKRYDEHHQDLTLLKALVRQQLPEKYKEIFFDQSKNGYAGYI
DGGASQEEFYKFIKPILEKMDGTEELLVKLNREDLLRKQRTFDNG
SIPHQIHLGELHAILRRQEDFYPFLKDNREKIEKILTFRIPYYVG
PLARGNSRFAWMTRKSEETITPWNFEEVVDKGASAQSFIERMTNF
DKNLPNEKVLPKHSLLYEYFTVYNELTKVKYVTEGMRKPAFLSGE
QKKAIVDLLFKTNRKVTVKQLKEDYFKKIECFDSVEISGVEDRFN
ASLGTYHDLLKIIKDKDFLDNEENEDILEDIVLTLTLFEDREMIE
ERLKTYAHLFDDKVMKQLKRRRYTGWGRLSRKLINGIRDKQSGKT
ILDFLKSDGFANRNFMQLIHDDSLTFKEDIQKAQVSGQGDSLHEH
IANLAGSPAIKKGILQTVKWDELVKVMGRHKPENIVIEMARENQT
TQKGQKNSRERMKRIEEGIKELGSQILKEHPVENTQLQNEKLYLY
YLQNGRDMYVDQELDINRLSDYDVDHIVPQSFLKDDSIDNKVLTR
SDKNRGKSDNVPSEEWKKMKNYWRQLLNAKLITQRKFDNLTKAER
GGLSELDKAGFIKRQLVETRQITKHVAQILDSRMNTKYDENDKLI
REVKVITLKSKLVSDFRKDFQFYKVREINNYHHAHDAYLNAVVGT
ALIKKYPKLESEFVYGDYKVYDVRKMIAKSEQEIGKATAKYFFYS
SGGSKRANDGSEFEPKKKRKV
ABE8e-CP1041 monomer editor: NLS, linker,
TadA*, CP1041
(SEQ ID NO: 218)
MKRTADGSEFESPKKKRKVSEVEFSHEYWMRHALTLAKRARDERE
VPVGAVLVLNNRVIGEGWNRAIGLHDPTAHAEIMALRQGGLVMQN
YRLIDATLYVTFEPCVMCAGAMIHSRIGRVVFGVRNSKRGAAGSL
MNVLNYPGMNHRVEITEGILADECAALLCDFYRMPRQVFNAQKKA
QSSINSGGSSGGSSGSETPGTSESATPESSGGSSGGSNIMNFFKT
EITLANGEIRKRPLIETNGETGEIVWDKGRDFATVRKVLSMPQVN
IVKKTEVQTGGFSKESILPKRNSDKLIARKKDWDPKKYGGFDSPT
VAYSVLVVAKVEKGKSKKLKSVKELLGITIMERSSFEKNPIDFLE
AKGYKEVKKDLIIKLPKYSLFELENGRKRMLASAGELQKGNELAL
PSKYVNFLYLASHYEKLKGSPEDNEQKQLFVEQHKHYLDEIIEQI
SEFSKRVILADANLDKVLSAYNKHRDKPIREQAENIIHLFTLTNL
GAPAAFKYFDTTIDRKRYTSTKEVLDATLIHQSITGLYETRIDLS
QLGGDGGSGGSGGSGGSGGSGGSGGDKKYSIGLAIGTNSVGWAVI
TDEYKVPSKKFKVLGNTDRHSIKKNLIGALLFDSGETAEATRLKR
TARRRYTRRKNRICYLQEIFSNEMAKVDDSFFHRLEESFLVEEDK
KHERHPIFGNIVDEVAYHEKYPTIYHLRKKLVDSTDKADLRLIYL
ALAHMIKFRGHFLIEGDLNPDNSDVDKLFIQLVQTYNQLFEENPI
NASGVDAKAILSARLSKSRRLENLIAQLPGEKKNGLFGNLIALSL
GLTPNFKSNFDLAEDAKLQLSKDTYDDDLDNLLAQIGDQYADLFL
AAKNLSDAILLSDILRVNTEITKAPLSASMIKRYDEHHQDLTLLK
ALVRQQLPEKYKEIFFDQSKNGYAGYIDGGASQEEFYKFIKPILE
KMDGTEELLVKLNREDLLRKQRTFDNGSIPHQIHLGELHAILRRQ
EDFYPFLKDNREKIEKILTFRIPYYVGPLARGNSRFAWMTRKSEE
TITPWNFEEVVDKGASAQSFIERMTNFDKNLPNEKVLPKHSLLYE
YFTVYNELTKVKYVTEGMRKPAFLSGEQKKAIVDLLFKTNRKVTV
KQLKEDYFKKIECFDSVEISGVEDRFNASLGTYHDLLKIIKDKDF
LDNEENEDILEDIVLTLTLFEDREMIEERLKTYAHLFDDKVMKQL
KRRRYTGWGRLSRKLINGIRDKQSGKTILDFLKSDGFANRNFMQL
IHDDSLTFKEDIQKAQVSGQGDSLHEHIANLAGSPAIKKGILQTV
KVVDELVKVMGRHKPENIVIEMARENQTTQKGQKNSRERMKRIEE
GIKELGSQILKEHPVENTQLQNEKLYLYYLQNGRDMYVDQELDIN
RLSDYDVDHIVPQSFLKDDSIDNKVLTRSDKNRGKSDNVPSEEVV
KKMKNYWRQLLNAKLITQRKFDNLTKAERGGLSELDKAGFIKRQL
VETRQITKHVAQILDSRMNTKYDENDKLIREVKVITLKSKLVSDF
RKDFQFYKVREINNYHHAHDAYLNAVVGTALIKKYPKLESEFVYG
DYKVYDVRKMIAKSEQEIGKATAKYFFYSSGGSKRTADGSEFEPK
KKRKV
ABE8e-CP1028 dimer editor: NLS, wtTadA,
linker, TadA*, CP 1028
(SEQ ID NO: 217)
MKRTADGSEFESPKKKRKVSEVEFSHEYWMRHALTLAKRAWDERE
VPVGAVLVHNNRVIGEGWNRPIGRHDPTAHAEIMALRQGGLVMQN
YRLIDATLYVTLEPCVMCAGAMIHSRIGRVVFGARDAKTGAAGSL
MDVLHHPGMNHRVEITEGILADECAALLSDFFRMRRQEIKAQKKA
QSSTDSGGSSGGSSGSETPGTSESATPESSGGSSGGSSEVEFSHE
YWMRHALTLAKRARDEREVPVGAVLVLNNRVIGEGWNRAIGLHDP
TAHAEIMALRQGGLVMQNYRLIDATLYVTFEPCVMCAGAMIHSRI
GRVVFGVRNSKRGAAGSEMNVLNYPGMNHRVEITEGILADECAAL
LCDFYRMPRQVFNAQKKAQSSINSGGSSGGSSGSEEPGESESATP
ESSGGSSGGSEIGKATAKYFFYSNIMNFFKTEITLANGEIRKRPL
IETNGETGEIVWDKGRDFATVRKVLSMPQVNIVKKTEVQTGGFSK
ESILPKRNSDKLIARKKDWDPKKYGGFDSPTVAYSVLWAKVEKGK
SKKLKSVKELLGITIMERSSFEKNPIDFLEAKGYKEVKKDLIIKL
PKYSLFELENGRKRMLASAGELQKGNELALPSKYVNFLYLASHYE
KLKGSPEDNEQKQLFVEQHKHYLDEIIEQISEFSKRVILADANLD
KVLSAYNKHRDKPIREQAENIIHLFTLTNLGAPAAFKYFDTTIDR
KRYTSTKEVLDATLIHQSITGLYETRIDLSQLGGDGGSGGSGGSG
GSGGSGGSGGMDKKYSIGLAIGTNSVGWAVITDEYKVPSKKFKVL
GNTDRHSIKKNLIGALLFDSGETAEATRLKRTARRRYTRRKNRIC
YLQEIFSNEMAKVDDSFFHRLEESFLVEEDKKHERHPIFGNIVDE
VAYHEKYPTIYHLRKKLVDSTDKADLRLIYLALAHMIKFRGHFLI
EGDLNPDNSDVDKLFIQLVQTYNQLFEENPINASGVDAKAILSAR
LSKSRRLENLIAQLPGEKKNGLFGNLIALSLGLTPNFKSNFDLAE
DAKLQLSKDTYDDDLDNLLAQIGDQYADLFLAAKNLSDAILLSDI
LRVNTEITKAPLSASMIKRYDEHHQDLTLLKALVRQQLPEKYKEI
FFDQSKNGYAGYIDGGASQEEFYKFIKPILEKMDGTEELLVKLNR
EDLLRKQRTFDNGSIPHQIHLGELHAILRRQEDFYPFLKDNREKI
EKILTFRIPYYVGPLARGNSRFAWMTRKSEETITPWNFEEVVDKG
ASAQSFIERMTNFDKNLPNEKVLPKHSLLYEYFTVYNELTKVKYV
TEGMRKPAFLSGEQKKAIVDLLFKTNRKVTVKQLKEDYFKKIECF
DSVEISGVEDRFNASLGTYHDLLKIIKDKDFLDNEENEDILEDIV
LTLTLFEDREMIEERLKTYAHLFDDKVMKQLKRRRYTGWGRLSRK
LINGIRDKQSGKTILDFLKSDGFANRNFMQLIHDDSLTFKEDIQK
AQVSGQGDSLHEHIANLAGSPAIKKGILQTVKWDELVKVMGRHKP
ENIVIEMARENQTTQKGQKNSRERMKRIEEGIKELGSQILKEHPV
ENTQLQNEKLYLYYLQNGRDMYVDQELDINRLSDYDVDHIVPQSF
LKDDSIDNKVLTRSDKNRGKSDNVPSEEWKKMKNYWRQLLNAKLI
TQRKFDNLTKAERGGLSELDKAGFIKRQLVETRQITKHVAQILDS
RMNTKYDENDKLIREVKVITLKSKLVSDFRKDFQFYKVREINNYH
HAHDAYLNAWGTALIKKYPKLESEFVYGDYKVYDVRKMIAKSEQ
SGGSKRTADGSEFEPKKKRKV
ABE8e-CP1028 monomer editor: NLS, linker,
TadA*, CP 1028
(SEQ ID NO: 216)
MKRTADGSEFESPKKKRKVSEVEFSHEYWMRHALTLAKRARDERE
VPVGAVLVLNNRVIGEGWNRAIGLHDPTAHAEIMALRQGGLVMQN
YRLIDATLYVTFEPCVMCAGAMIHSRIGRVVFGVRNSKRGAAGSL
MNVLNYPGMNHRVEITEGILADECAALLCDFYRMPRQVFNAQKKA
QSSINSGGSSGGSSGSETPGTSESATPESSGGSSGGSEIGKATAK
YFFYSNIMNFFKTEITLANGEIRKRPLIETNGE
TGEIVWDKGRDFATVRKVLSMPQVNIVKKTEVQTGGFSKESILPK
RNSDKLIARKKDWDPKKYGGFDSPTVAYSVLVVAKVEKGKSKKLK
SVKELLGITIMERSSFEKNPIDFLEAKGYKEVKKDLIKLPKYSLF
ELENGRKRMLASAGELQKGNELALPSKYVNFLYLASHYEKLKGSP
EDNEQKQLFVEQHKHYLDEHIEQISEFSKRVILADANLDKVLSAY
NKHRDKPIREQAENIIHLFTLTNLGAPAAFKYFDTTIDRKRYTST
KEVLDATLIHQSITGLYETRIDLSQLGGDGGSGGSGGSGGSGGSG
GSGGMDKKYSIGLAIGTNSVGWAVITDEYKVPSKKFKVLGNTDRH
SIKKNLIGALLFDSGETAEATRLKRTARRRYTRRKNRICYLQEIF
SNEMAKVDDSFFHRLEESFLVEEDKKHERHPIFGNIVDEVAYHEK
YPTIYHLRKKLVDSTDKADLRLIYLALAHMIKFRGHFLIEGDLNP
DNSDVDKLFIQLVQTYNQLFEENPINASGVDAKAILSARLSKSRR
LENLIAQLPGEKKNGLFGNLIALSLGLTPNFKSNFDLAEDAKLQL
SKDTYDDDLDNLLAQIGDQYADLFLAAKNLSDAILLSDILRVNTE
ITKAPLSASMIKRYDEHHQDLTLLKALVRQQLPEKYKEIFFDQSK
NGYAGYIDGGASQEEFYKFIKPILEKMDGTEELLVKLNREDLLRK
QRTFDNGSIPHQIHLGELHAILRRQEDFYPFLKDNREKIEKILTF
RIPYYVGPLARGNSRFAWMTRKSEETITPWNFEEVVDKGASAQSF
IERMTNFDKNLPNEKVLPKHSLLYEYFTVYNELTKVKYVTEGMRK
PAFLSGEQKKAIVDLLFKTNRKVTVKQLKEDYFKKIECFDSVEIS
GVEDRFNASLGTYHDLLKIIKDKDFLDNEENEDILEDIVLTLTLF
EDREMIEERLKTYAHLFDDKVMKQLKRRRYTGWGRLSRKLINGIR
DKQSGKTILDFLKSDGFANRNFMQLIHDDSLTFKEDIQKAQVSGQ
GDSLHEHIANLAGSPAIKKGILQTVKVVDELVKVMGRHKPENIVI
EMARENQTTQKGQKNSRERMKRIEEGIKELGSQILKEHPVENTQL
QNEKLYLYYLQNGRDMYVDQELDINRLSDYDVDHIVPQSFLKDDS
IDNKVLTRSDKNRGKSDNVPSEEVVKKMKNYWRQLLNAKLITQRK
FDNLTKAERGGLSELDKAGFIKRQLVETRQITKHVAQILDSRMNT
KYDENDKLIREVKVITLKSKLVSDFRKDFQFYKVREINNYHHAHD
AYLNAVVGTALIKKYPKLESEFVYGDYKVYDVRKMIAKSEQSGGS
KRTADGSEFEPKKKRKV
ABE8e-VRQR dimer editor: NLS, wtTadA,
linker, TadA*, SpCas9-VRQR
(SEQ ID NO: 3253)
MKRTADGSEFESPKKKRKVSEVEFSHEYWMRHALTLAKRAWDERE
VPVGAVLVHNNRVIGEGWNRPIGRHDPTAHAEIMALRQGGLVMQN
YRLIDATLYVTLEPCVMCAGAMIHSRIGRVVFGARDAKTGAAGSL
MDVLHHPGMNHRVEITEGILADECAALLSDFFRMRRQEIKAQKKA
QSSTDSGGSSGGSSGSETPGTSESATPESSGGSSGGSSEVEFSHE
YWMRHALTLAKRARDEREVPVGAVLVLNNRVIGEGWNRAIGLHDP
TAHAEIMALRQGGLVMQNYRLIDATLYVTFEPCVMCAGAMIHSRI
GRVVFGVRNSKRGAAGSEMNVLNYPGMNHRVEITEGILADECAAL
LCDFYRMPRQVFNAQKKAQSSINSGGSSGGSSGSETPGTSESATP
ESSGGSSGGSDKKYSIGLAIGTNSVGWAVITDEYKVPSKKFKVLG
NTDRHSIKKNLIGALLFDSGETAEATRLKRTARRRYTRRKNRICY
LQEIFSNEMAKVDDSFFHRLEESFLVEEDKKHERHPIFGNIVDEV
AYHEKYPTIYHLRKKLVDSTDKADLRLIYLALAHMIKFRGHFLIE
GDLNPDNSDVDKLFIQLVQTYNQLFEENPINASGVDAKAILSARL
SKSRRLENLIAQLPGEKKNGLFGNLIALSLGLTPNFKSNFDLAED
AKLQLSKDTYDDDLDNLLAQIGDQYADLFLAAKNLSDAILLSDIL
RVNTEITKAPLSASMIKRYDEHHQDLTLLKALVRQQLPEKYKEIF
FDQSKNGYAGYIDGGASQEEFYKFIKPILEKMDGTEELLVKLNRE
DLLRKQRTFDNGSIPHQIHLGELHAILRRQEDFYPFLKDNREKIE
KILTFRIPYYVGPLARGNSRFAWMTRKSEETITPWNFEEVVDKGA
SAQSFIERMTNFDKNLPNEKVLPKHSLLYEYFTVYNELTKVKYVT
EGMRKPAFLSGEQKKAIVDLLFKTNRKVTVKQLKEDYFKKIECFD
SVEISGVEDRFNASLGTYHDLLKIIKDKDFLDNEENEDILEDIVL
TLTLFEDREMIEERLKTYAHLFDDKVMKQLKRRRYTGWGRLSRKL
INGIRDKQSGKTILDFLKSDGFANRNFMQLIHDDSLTFKEDIQKA
QVSGQGDSLHEHIANLAGSPAIKKGILQTVKWDELVKVMGRHKPE
NIVIEMARENQTTQKGQKNSRERMKRIEEGIKELGSQILKEHPVE
NTQLQNEKLYLYYLQNGRDMYVDQELDINRLSDYDVDHIVPQSFL
KDDSIDNKVLTRSDKNRGKSDNVPSEEWKKMKNYWRQLLNAKLIT
QRKFDNLTKAERGGLSELDKAGFIKRQLVETRQITKHVAQILDSR
MNTKYDENDKLIREVKVITLKSKLVSDFRKDFQFYKVREINNYHH
AHDAYLNAWGTALIKKYPKLESEFVYGDYKVYDVRKMIAKSEQEI
GKATAKYFFYSNIMNFFKTEITLANGEIRKRPLIETNGETGEIVW
DKGRDFATVRKVLSMPQVNIVKKTEVQTGGFSKESILPKRNSDKL
IARKKDWDPKKYGGFVSPTVAYSVLVVAKVEKGKSKKLKSVKELL
GITIMERSSFEKNPIDFLEAKGYKEVKKDLIIKLPKYSLFELENG
RKRMLASARELQKGNELALPSKYVNFLYLASHYEKLKGSPEDNEQ
KQLFVEQHKHYLDEIIEQISEFSKRVILADANLDKVLSAYNKHRD
KPIREQAENIIHLFTLTNLGAPAAFKYFDTTIDRKQYRSTKEVLD
ATLIHQSITGLYETRIDLSQLGGDSGGSKRTADGSEFEPKKKRKV
ABE8e-VRQR monomer editor: NLS, linker,
TadA*, SpCas9-VRQR
(SEQ ID NO: 3254)
MKRTADGSEFESPKKKRKVSEVEFSHEYWMRHALTLAKRARDERE
VPVGAVLVLNNRVIGEGWNRAIGLHDPTAHAEIMALRQGGLVMQN
YRLIDATLYVTFEPCVMCAGAMIHSRIGRVVFGVRNSKRGAAGSL
MNVLNYPGMNHRVEITEGILADECAALLCDFYRMPRQVFNAQKKA
QSSINSGGSSGGSSGSETPGTSESATPESSGGSSGGSDKKYSIGL
AIGTNSVGWAVITDEYKVPSKKFKVLGNTDRHSIKKNLIGALLFD
SGETAEATRLKRTARRRYTRRKNRICYLQEIFSNEMAKVDDSFFH
RLEESFLVEEDKKHERHPIFGNIVDEVAYHEKYPTIYHLRKKLVD
STDKADLRLIYLALAHMIKFRGHFLIEGDLNPDNSDVDKLFIQLV
QTYNQLFEENPINASGVDAKAILSARLSKSRRLENLIAQLPGEKK
NGLFGNLIALSLGLTPNFKSNFDLAEDAKLQLSKDTYDDDLDNLL
AQIGDQYADLFLAAKNLSDAILLSDILRVNTEITKAPLSASMIKR
YDEHHQDLTLLKALVRQQLPEKYKEIFFDQSKNGYAGYIDGGASQ
EEFYKFIKPILEKMDGTEELLVKLNREDLLRKQRTFDNGSIPHQI
HLGELHAILRRQEDFYPFLKDNREKIEKILTFRIPYYVGPLARGN
SRFAWMTRKSEETITPWNFEEVVDKGASAQSFIERMTNFDKNLPN
EKVLPKHSLLYEYFTVYNELTKVKYVTEGMRKPAFLSGEQKKAIV
DLLFKTNRKVTVKQLKEDYFKKIECFDSVEISGVEDRFNASLGTY
HDLLKIIKDKDFLDNEENEDILEDIVLTLTLFEDREMIEERLKTY
AHLFDDKVMKQLKRRRYTGWGRLSRKLINGIRDKQSGKTILDFLK
SDGFANRNFMQLIHDDSLTFKEDIQKAQVSGQGDSLHEHIANLAG
SPAIKKGILQTVKVVDELVKVMGRHKPENIVIEMARENQTTQKGQ
KNSRERMKRIEEGIKELGSQILKEHPVENTQLQNEKLYLYYLQNG
RDMYVDQELDINRLSDYDVDHIVPQSFLKDDSIDNKVLTRSDKNR
GKSDNVPSEEVVKKMKNYWRQLLNAKLITQRKFDNLTKAERGGLS
ELDKAGFIKRQLVETRQITKHVAQILDSRMNTKYDENDKLIREVK
VITLKSKLVSDFRKDFQFYKVREINNYHHAHDAYLNAVVGTALIK
KYPKLESEFVYGDYKVYDVRKMIAKSEQEIGKATAKYFFYSNIMN
FFKTEITLANGEIRKRPLIETNGETGEIVWDKGRDFATVRKVLSM
PQVNIVKKTEVQTGGFSKESILPKRNSDKLIARKKDWDPKKYGGF
VSPTVAYSVLVVAKVEKGKSKKLKSVKELLGITIMERSSFEKNPI
DFLEAKGYKEVKKDLIIKLPKYSLFELENGRKRMLASARELQKGN
ELALPSKYVNFLYLASHYEKLKGSPEDNEQKQLFVEQHKHYLDEI
IEQISEFSKRVILADANLDKVLSAYNKHRDKPIREQAENIIHLFT
LTNLGAPAAFKYFDTTIDRKQYRSTKEVLDATLIHQSITGLYETR
IDLSQLGGDSGGSKRTADGSEFEPKKKRKV
ABE8e-NG-CP1041 dimer editor: NLS, wtTadA,
linker, TadA*, SpCas9-NG-
CP1041
(SEQ ID NO: 3244)
MKRTADGSEFESPKKKRKVSEVEFSHEYWMRHALTLAKRAWDERE
VPVGAVLVHNNRVIGEGWNRPIGRHDPTAHAEIMALRQGGLVMQN
YRLIDATLYVTLEPCVMCAGAMIHSRIGRVVFGARDAKTGAAGSL
MDVLHHPGMNHRVEITEGILADECAALLSDFFRMRRQEIKAQKKA
QSSTDSGGSSGGSSGSETPGTSESATPESSGGSSGGSSEVEFSHE
YWMRHALTLAKRARDEREVPVGAVLVLNNRVIGEGWNRAIGLHDP
TAHAEIMALRQGGLVMQNYRLIDATLYVTFEPCVMCAGAMIHSRI
GRVVFGVRNSKRGAAGSEMNVLNYPGMNHRVEITEGILADECAAL
LCDFYRMPRQVFNAQKKAQSSINSGGSSGGSSGSETPGESESATP
ESSGGSSGGSNIMNFFKTEITLANGEIRKRPLIETNGETGEIVWD
KGRDFATVRKVLSMPQVNIVKKTEVQTGGFSKESIRPKRNSDKLI
ARKKDWDPKKYGGFVSPTVAYSVLVVAKVEKGKSKKLKSVKELLG
ITIMERSSFEKNPIDFLEAKGYKEVKKDLIIKLPKYSLFELENGR
KRMLASARFLQKGNELALPSKYVNFLYLASHYEKLKGSPEDNEQK
QLFVEQHKHYLDEIIEQISEFSKRVILADANLDKVLSAYNKHRDK
PIREQAENIIHLFTLTNLGAPRAFKYFDTTIDRKVYRSTKEVLDA
TLIHQSITGLYETRIDLSQLGGDGGSGGSGGSGGSGGSGGSGGDK
KYSIGLAIGTNSVGWAVITDEYKVPSKKFKVLGNTDRHSIKKNLI
GALLFDSGETAEATRLKRTARRRYTRRKNRICYLQEIFSNEMAKV
DDSFFHRLEESFLVEEDKKHERHPIFGNIVDEVAYHEKYPTIYHL
RKKLVDSTDKADLRLIYLALAHMIKFRGHFLIEGDLNPDNSDVDK
LFIQLVQTYNQLFEENPINASGVDAKAILSARLSKSRRLENLIAQ
LPGEKKNGLFGNLIALSLGLTPNFKSNFDLAEDAKLQLSKDTYDD
DLDNLLAQIGDQYADLFLAAKNLSDAILLSDILRVNTEITKAPLS
ASMIKRYDEHHQDLTLLKALVRQQLPEKYKEIFFDQSKNGYAGYI
DGGASQEEFYKFIKPILEKMDGTEELLVKLNREDLLRKQRTFDNG
SIPHQIHLGELHAILRRQEDFYPFLKDNREKIEKILTFRIPYYVG
PLARGNSRFAWMTRKSEETITPWNFEEVVDKGASAQSFIERMTNF
DKNLPNEKVLPKHSLLYEYFTVYNELTKVKYVTEGMRKPAFLSGE
QKKAIVDLLFKTNRKVTVKQLKEDYFKKIECFDSVEISGVEDRFN
ASLGTYHDLLKIIKDKDFLDNEENEDILEDIVLTLTLFEDREMIE
ERLKTYAHLFDDKVMKQLKRRRYTGWGRLSRKLINGIRDKQSGKT
ILDFLKSDGFANRNFMQLIHDDSLTFKEDIQKAQVSGQGDSLHEH
IANLAGSPAIKKGILQTVKWDELVKVMGRHKPENIVIEMARENQT
TQKGQKNSRERMKRIEEGIKELGSQILKEHPVENTQLQNEKLYLY
YLQNGRDMYVDQELDINRLSDYDVDHIVPQSFLKDDSIDNKVLTR
SDKNRGKSDNVPSEEWKKMKNYWRQLLNAKLITQRKFDNLTKAER
GGLSELDKAGFIKRQLVETRQITKHVAQILDSRMNTKYDENDKLI
REVKVITLKSKLVSDFRKDFQFYKVREINNYHHAHDAYLNAWGTA
LIKKYPKLESEFVYGDYKVYDVRKMIAKSEQEIGKATAKYFFYSS
GGSKRTADGSEFEPKKKRKV
ABE8e-NG-CP1041 monomer editor: NLS,
linker, TadA*, SpCas9-NG-CP1041
(SEQ ID NO: 3245)
MKRTADGSEFESPKKKRKVSEVEFSHEYWMRHALTLAKRARDERE
VPVGAVLVLNNRVIGEGWNRAIGLHDPTAHAEIMALRQGGLVMQN
YRLIDATLYVTFEPCVMCAGAMIHSRIGRVVFGVRNSKRGAAGSL
MNVLNYPGMNHRVEITEGILADECAALLCDFYRMPRQVFNAQKKA
QSSINSGGSSGGSSGSETPGTSESATPESSGGSSGGSNIMNFFKT
EITLANGEIRKRPLIETNGETGEIVWDKGRDFATVRKVLSMPQVN
IVKKTEVQTGGFSKESIRPKRNSDKLIARKKDWDPKKYGGFVSPT
VAYSVLVVAKVEKGKSKKLKSVKELLGITIMERSSFEKNPIDFLE
AKGYKEVKKDLIIKLPKYSLFELENGRKRMLASARFLQKGNELAL
PSKYVNFLYLASHYEKLKGSPEDNEQKQLFVEQHKHYLDEIIEQI
SEFSKRVILADANLDKVLSAYNKHRDKPIREQAENIIHLFTLTNL
GAPRAFKYFDTTIDRKVYRSTKEVLDATLIHQSITGLYETRIDLS
QLGGDGGSGGSGGSGGSGGSGGSGGDKKYSIGLAIGTNSVGWAVI
TDEYKVPSKKFKVLGNTDRHSIKKNLIGALLFDSGETAEATRLKR
TARRRYTRRKNRICYLQEIFSNEMAKVDDSFFHRLEESFLVEEDK
KHERHPIFGNIVDEVAYHEKYPTIYHLRKKLVDSTDKADLRLIYL
ALAHMIKFRGHFLIEGDLNPDNSDVDKLFIQLVQTYNQLFEENPI
NASGVDAKAILSARLSKSRRLENLIAQLPGEKKNGLFGNLIALSL
GLTPNFKSNFDLAEDAKLQLSKDTYDDDLDNLLAQIGDQYADLFL
AAKNLSDAILLSDILRVNTEITKAPLSASMIKRYDEHHQDLTLLK
ALVRQQLPEKYKEIFFDQSKNGYAGYIDGGASQEEFYKFIKPILE
KMDGTEELLVKLNREDLLRKQRTFDNGSIPHQIHLGELHAILRRQ
EDFYPFLKDNREKIEKILTFRIPYYVGPLARGNSRFAWMTRKSEE
TITPWNFEEVVDKGASAQSFIERMTNFDKNLPNEKVLPKHSLLYE
YFTVYNELTKVKYVTEGMRKPAFLSGEQKKAIVDLLFKTNRKVTV
KQLKEDYFKKIECFDSVEISGVEDRFNASLGTYHDLLKIIKDKDF
LDNEENEDILEDIVLTLTLFEDREMIEERLKTYAHLFDDKVMKQL
KRRRYTGWGRLSRKLINGIRDKQSGKTILDFLKSDGFANRNFMQL
IHDDSLTFKEDIQKAQVSGQGDSLHEHIANLAGSPAIKKGILQTV
KVVDELVKVMGRHKPENIVIEMARENQTTQKGQKNSRERMKRIEE
GIKELGSQILKEHPVENTQLQNEKLYLYYLQNGRDMYVDQELDIN
RLSDYDVDHIVPQSFLKDDSIDNKVLTRSDKNRGKSDNVPSEEVV
KKMKNYWRQLLNAKLITQRKFDNLTKAERGGLSELDKAGFIKRQL
VETRQITKHVAQILDSRMNTKYDENDKLIREVKVITLKSKLVSDF
RKDFQFYKVREINNYHHAHDAYLNAVVGTALIKKYPKLESEFVYG
DYKVYDVRKMIAKSEQEIGKATAKYFFYSSGGSKRTADGSEFEPK
KKRKV
ABE8e-iSpyMac dimer editor: NLS, wtTadA,
linker, TadA*, SpCas9-iSpyMac
(SEQ ID NO: 3255)
MKRTADGSEFESPKKKRKVSEVEFSHEYWMRHALTLAKRAWDERE
VPVGAVLVHNNRVIGEGWNRPIGRHDPTAHAEIMALRQGGLVMQN
YRLIDATLYVTLEPCVMCAGAMIHSRIGRVVFGARDAKTGAAGSL
MDVLHHPGMNHRVEITEGILADECAALLSDFFRMRRQEIKAQKKA
QSSTDSGGSSGGSSGSETPGTSESATPESSGGSSGGSSEVEFSHE
YWMRHALTLAKRARDEREVPVGAVLVLNNRVIGEGWNRAIGLHDP
TAHAEIMALRQGGLVMQNYRLIDATLYVTFEPCVMCAGAMIHSRI
GRVVFGVRNSKRGAAGSEMNVLNYPGMNHRVEITEGILADECAAL
LCDFYRMPRQVFNAQKKAQSSINSGGSSGGSSGSETPGTSESATP
ESSGGSSGGSDKKYSIGLDIGTNSVGWAVITDEYKVPSKKFKVLG
NTDRHSIKKNLIGALLFDSGETAEATRLKRTARRRYTRRKNRICY
LQEIFSNEMAKVDDSFFHRLEESFLVEEDKKHERHPIFGNIVDEV
AYHEKYPTIYHLRKKLVDSTDKADLRLIYLALAHMIKFRGHFLIE
GDLNPDNSDVDKLFIQLVQTYNQLFEENPINASGVDAKAILSARL
SKSRRLENLIAQLPGEKKNGLFGNLIALSLGLTPNFKSNFDLAED
AKLQLSKDTYDDDLDNLLAQIGDQYADLFLAAKNLSDAILLSDIL
RVNTEITKAPLSASMIKRYDEHHQDLTLLKALVRQQLPEKYKEIF
FDQSKNGYAGYIDGGASQEEFYKFIKPILEKMDGTEELLVKLNRE
DLLRKQRTFDNGSIPHQIHLGELHAILRRQEDFYPFLKDNREKIE
KILTFRIPYYVGPLARGNSRFAWMTRKSEETITPWNFEEVVDKGA
SAQSFIERMTNFDKNLPNEKVLPKHSLLYEYFTVYNELTKVKYVT
EGMRKPAFLSGEQKKAIVDLLFKTNRKVTVKQLKEDYFKKIECFD
SVEISGVEDRFNASLGTYHDLLKIIKDKDFLDNEENEDILEDIVL
TLTLFEDREMIEERLKTYAHLFDDKVMKQLKRRRYTGWGRLSRKL
INGIRDKQSGKTILDFLKSDGFANRNFMQLIHDDSLTFKEDIQKA
QVSGQGDSLHEHIANLAGSPAIKKGILQTVKWDELVKVMGRHKPE
NIVIEMARENQTTQKGQKNSRERMKRIEEGIKELGSQILKEHPVE
NTQLQNEKLYLYYLQNGRDMYVDQELDINRLSDYDVDHIVPQSFL
KDDSIDNKVLTRSDKNRGKSDNVPSEEWKKMKNYWRQLLNAKLIT
QRKFDNLTKAERGGLSELDKAGFIKRQLVETRQITKHVAQILDSR
MNTKYDENDKLIREVKVITLKSKLVSDFRKDFQFYKVREINNYHH
AHDAYLNAWGTALIKKYPKLESEFVYGDYKVYDVRKMIAKSEQEI
GKATAKYFFYSNIMNFFKTEITLANGEIRKRPLIETNGETGEIVW
DKGRDFATVRKVLSMPQVNIVKKTESGGSKRTADGSEFEPKKKRK
V
ABE8e-iSpyMac monomer editor: NLS, linker,
TadA*, SpCas9-iSpyMac
(SEQ ID NO: 3256)
MKRTADGSEFESPKKKRKVSEVEFSHEYWMRHALTLAKRARDERE
VPVGAVLVLNNRVIGEGWNRAIGLHDPTAHAEIMALRQGGLVMQN
YRLIDATLYVTFEPCVMCAGAMIHSRIGRVVFGVRNSKRGAAGSL
MNVLNYPGMNHRVEITEGILADECAALLCDFYRMPRQVFNAQKKA
QSSINSGGSSGGSSGSETPGTSESATPESSGGSSGGSDKKYSIGL
DIGTNSVGWAVITDEYKVPSKKFKVLGNTDRHSIKKNLIGALLFD
SGETAEATRLKRTARRRYTRRKNRICYLQEIFSNEMAKVDDSFFH
RLEESFLVEEDKKHERHPIFGNIVDEVAYHEKYPTIYHLRKKLVD
STDKADLRLIYLALAHMIKFRGHFLIEGDLNPDNSDVDKLFIQLV
QTYNQLFEENPINASGVDAKAILSARLSKSRRLENLIAQLPGEKK
NGLFGNLIALSLGLTPNFKSNFDLAEDAKLQLSKDTYDDDLDNLL
AQIGDQYADLFLAAKNLSDAILLSDILRVNTEITKAPLSASMIKR
YDEHHQDLTLLKALVRQQLPEKYKEIFFDQSKNGYAGYIDGGASQ
EEFYKFIKPILEKMDGTEELLVKLNREDLLRKQRTFDNGSIPHQI
HLGELHAILRRQEDFYPFLKDNREKIEKILTFRIPYYVGPLARGN
SRFAWMTRKSEETITPWNFEEVVDKGASAQSFIERMTNFDKNLPN
EKVLPKHSLLYEYFTVYNELTKVKYVTEGMRKPAFLSGEQKKAIV
DLLFKTNRKVTVKQLKEDYFKKIECFDSVEISGVEDRFNASLGTY
HDLLKIIKDKDFLDNEENEDILEDIVLTLTLFEDREMIEERLKTY
AHLFDDKVMKQLKRRRYTGWGRLSRKLINGIRDKQSGKTILDFLK
SDGFANRNFMQLIHDDSLTFKEDIQKAQVSGQGDSLHEHIANLAG
SPAIKKGILQTVKVVDELVKVMGRHKPENIVIEMARENQTTQKGQ
KNSRERMKRIEEGIKELGSQILKEHPVENTQLQNEKLYLYYLQNG
RDMYVDQELDINRLSDYDVDHIVPQSFLKDDSIDNKVLTRSDKNR
GKSDNVPSEEVVKKMKNYWRQLLNAKLITQRKFDNLTKAERGGLS
ELDKAGFIKRQLVETRQITKHVAQILDSRMNTKYDENDKLIREVK
VITLKSKLVSDFRKDFQFYKVREINNYHHAHDAYLNAVVGTALIK
KYPKLESEFVYGDYKVYDVRKMIAKSEQEIGKATAKYFFYSNIMN
FFKTEITLANGEIRKRPLIETNGETGEIVWDKGRDFATVRKVLSM
PQVNIVKKTESGGSKRTADGSEFEPKKKRKV

### Additional Exemplary CBEs

In various embodiments, the present disclosure provides novel cytosine base editors (CBEs) comprising a napDNAbp domain and a cytosine deaminase domain that enzymatically deaminates a cytosine nucleobase of a C:G nucleobase pair to a uracil. The uracil may be subsequently converted to a thymine (T) by the cell's DNA repair and replication machinery. The mismatched guanine (G) on the opposite strand may subsequently be converted to an adenine (A) by the cell's DNA repair and replication machinery. In this manner, a target C:G nucleobase pair is ultimately converted to a T:A nucleobase pair.

The disclosed novel cytosine base editors exhibit increased on-target editing scope while maintaining minimized off-target DNA editing relative to existing CBEs. The CBEs described herein provide ˜10- to ˜100-fold lower average Cas9-independent off-target DNA editing, while maintaining efficient on-target editing at most positions targetable by existing CBEs. The disclosed CBEs comprise combinations of mutant cytosine deaminases, such as the YE1, YE2, YEE, and R33A deaminases, and Cas9 domains, and/or novel combinations of mutant cytosine deaminases, Cas9 domains, uracil glycosylase inhibitor (UGI) domains and nuclear localizations sequence (NLS) domains, relative to existing base editors. Existing base editors include BE3, which comprises the structure NH₂-[NLS]-[rAPOBEC1 deaminase]-[Cas9 nickase (D10A)]-[UGI domain]-[NLS]-COOH; BE4, which comprises the structure NH₂-[NLS]-[rAPOBEC1 deaminase]-[Cas9 nickase (D10A)]-[UGI domain]-[UGI domain]-[NLS]-COOH; and BE4max, which is a version of BE4 for which the codons of the base editor-encoding construct has been codon-optimized for expression in human cells.

Zuo et al. recently reported that, when overexpressed in mouse embryos and rice, BE3, the original CBE, induces an average random C:G-to-T:A mutation frequency of 5×10⁻⁸ per bp and 1.7×10⁻³ per bp, respectively. See “Cytosine base editor generates substantial off-target single-nucleotide variants in mouse embryos.” Science 364, 289-292 (2019), herein incorporated by reference. Editing was observed in sequences that had little to no similarity to the target sequences. These off-target edits may have arisen from the intrinsic DNA affinity of BE3's deaminase domain, independent of the guide RNA-programmed DNA binding of Cas9. See also Jin et al., Cytosine, but not adenine, base editors induce genome-wide off-target mutations in rice. Science 364, (2019), herein incorporated by reference.

Zuo et al. also found that Cas9-independent off-target editing events were enriched in transcribed regions of the genome, particularly in highly-expressed genes. Some of these were tumor suppressor genes. Accordingly, there is a need in the art to develop base editors that possess low off-target editing frequencies that may avoid undesired activation or inactivation of genes associated with diseases or disorders, such as cancer, and assays that rapidly measure the off-target editing frequencies of these base editors.

Exemplary CBEs may provide an off-target editing frequency of less than 2.0% after being contacted with a nucleic acid molecule comprising a target sequence, e.g., a target nucleobase pair. Further exemplary CBEs provide an off-target editing frequency of less than 1.5% after being contacted with a nucleic acid molecule comprising a target sequence comprising a target nucleobase pair. Further exemplary CBEs may provide an off-target editing frequency of less than 1.25%, less than 1.1%, less than 1%, less than 0.75%, less than 0.5%, less than 0.4%, less than 0.25%, less than 0.2%, less than 0.15%, less than 0.1%, less than 0.05%, or less than 0.025%, after being contacted with a nucleic acid molecule comprising a target sequence.

For instance, the cytosine base editors YE1-BE4, YE1-CP1028, YE1-SpCas9-NG (also referred to herein as YE1-NG), R33A-BE4, and R33A+K34A-BE4-CP1028, which are described below, may exhibit off-target editing frequencies of less than 0.75% (e.g., about 0.4% or less) while maintaining on-target editing efficiencies of about 60% or more, in target sequences in mammalian cells. Each of these base editors comprises modified cytosine deaminases (e.g., YE1, R33A, or R33A+K34A) and may further comprise a modified napDNAbp domain such as a circularly permuted Cas9 domain (e.g., CP1028) or a Cas9 domain with an expanded PAM window (e.g., SpCas9-NG). These five base editors may be the most preferred for applications in which off-target editing, and in particular Cas9-independent off-target editing, must be minimized. In particular, base editors comprising a YE1 deaminase domain provide efficient on-target editing with greatly decreased Cas9-independent editing, as confirmed by whole-genome sequencing.

Exemplary CBEs may further possess an on-target editing efficiency of more than 50% after being contacted with a nucleic acid molecule comprising a target sequence. Further exemplary CBEs possess an on-target editing efficiency of more than 60% after being contacted with a nucleic acid molecule comprising a target sequence. Further exemplary CBEs possess an on-target editing efficiency of more than 65%, more than 70%, more than 75%, more than 80%, more than 82.5%, or more than 85% after being contacted with a nucleic acid molecule comprising a target sequence.

The disclosed CBEs may exhibit indel frequencies of less than 0.75%, less than 0.6%, less than 0.5%, less than 0.4%, less than 0.3%, or less than 0.2% after being contacted with a nucleic acid molecule containing a target sequence. The disclosed CBEs may further exhibit reduced RNA off-target editing relative to existing CBEs. The disclosed CBEs may further result in increased product purity after being contacted with a nucleic acid molecule containing a target sequence relative to existing CBEs.

The disclosed CBEs may further comprise one or more nuclear localization signals (NLSs) and/or two or more uracil glycosylase inhibitor (UGI) domains. Thus, the base editors may comprise the structure: NH₂-[first nuclear localization sequence]-[cytosine deaminase domain]-[napDNAbp domain]-[first UGI domain]-[second UGI domain]-[second nuclear localization sequence]-COOH, wherein each instance of “]-[” indicates the presence of an optional linker sequence. Exemplary CBEs may have a structure that comprises the “BE4max” architecture, with an NH₂-[NLS]-[cytosine deaminase]-[Cas9 nickase]-[UGI domain]-[UGI domain]-[NLS]-COOH structure, having optimized nuclear localization signals and wherein the napDNAbp domain comprises a Cas9 nickase. This BE4max structure was reported to have optimized codon usage for expression in human cells, as reported in Koblan et al., Nat Biotechnol. 2018; 36(9):843-846, herein incorporated by reference.

In other embodiments, exemplary CBEs may have a structure that comprises a modified BE4max architecture that contains a napDNAbp domain comprising a Cas9 variant other than Cas9 nickase, such as SpCas9-NG, xCas9, or circular permutant CP1028. Accordingly, exemplary CBEs may comprise the structure: NH₂-[NLS]-[cytosine deaminase]-[CP1028]-[UGI domain]-[UGI domain]-[NLS]-COOH; NH₂-[NLS]-[cytosine deaminase]-[xCas9]-[UGI domain]-[UGI domain]-[NLS]-COOH; or NH₂-[NLS]-[cytosine deaminase]-[SpCas9-NG]-[UGI domain]-[UGI domain]-[NLS]-COOH, wherein each instance of “]-[” indicates the presence of an optional linker sequence.

The disclosed CBEs may comprise modified (or evolved) cytosine deaminase domains, such as deaminase domains that recognize an expanded PAM sequence, have improved efficiency of deaminating 5′-GC targets, and/or make edits in a narrower target window. In some embodiments, the disclosed cytosine base editors comprise evolved nucleic acid programmable DNA binding proteins (napDNAbp), such as an evolved Cas9.

Exemplary cytosine base editors comprise sequences that are at least 85%, at least 90%, at least 95%, at least 98%, at least 99%, or at least 99.5% identical to the following amino acid sequences, SEQ ID NOs: 223-248.

Where indicated, “—BE4” refers to the BE4max architecture, or NH₂-[first nuclear localization sequence]-[cytosine deaminase domain]-[32aa linker]-[SpCas9 nickase (nCas9, or nSpCas9) domain]-[9aa linker]-[first UGI domain]-[9aa-linker]-[second UGI domain]-[second nuclear localization sequence]-COOH. Where indicated, “BE4max, modified with SpCas9-NG” and “—SpCas9-NG” refer to a modified BE4max architecture in which the SpCas9 nickase domain has been replaced with an SpCas9-NG, i.e., NH₂-[first nuclear localization sequence]-[cytosine deaminase domain]-[32aa linker]-[SpCas9-NG]-[9aa linker]-[first UGI domain]-[9aa-linker]-[second UGI domain]-[second nuclear localization sequence]-COOH. And where indicated, “BE4-CP1028” refers to a modified BE4max architecture in which the Cas9 nickase domain has been replaced with a S. pyogenes CP1028, i.e., NH₂-[first nuclear localization sequence]-[cytosine deaminase domain]-[32aa linker]-[CP1028]-[9aa linker]-[first UGI domain]-[9aa-linker]-[second UGI domain]-[second nuclear localization sequence]-COOH.

As discussed above, preferred base editors comprise modified cytosine deaminases (e.g., YE1, R33A, or R33A+K34A) and may further comprise a modified napDNAbp domain such as a circularly permuted Cas9 domain (e.g., CP1028) or a Cas9 domain with an expanded PAM window (e.g., SpCas9-NG). The napDNAbp domains in the following amino acid sequences are indicated in italics.

BE4max
(SEQ ID NO: 223)
MKRTADGSEFESPKKKRKVSSETGPVAVDPTLRRRIEPHEFEVFFDPRELRKETCLLY
EINWGGRHSIWRHTSQNTNKHVEVNFIEKFTTERYFCPNTRCSITWFLSWSPCGECSR
AITEFLSRYPHVTLFIYIARLYHHADPRNRQGLRDLISSGVTIQIMTEQESGYCWRNFV
NYSPSNEAHWPRYPHLWVRLYVLELYCIILGLPPCLNILRRKQPQLTFFTIALQSCHY
QRLPPHILWATGLKSGGSSGGSSGSETPGTSESATPESSGGSSGGSDKKYSIGLAIGTNS
VGWAVITDEYKVPSKKFKVLGNTDRHSIKKNLIGALLFDSGETAEATRLKRTARRRYTRRK
NRICYLQEIFSNEMAKVDDSFFHRLEESFLVEEDKKHERHPIFGNIVDEVAYHEKYPTIYHL
RKKLVDSTDKADLRLIYLALAHMIKFRGHFLIEGDLNPDNSDVDKLFIQLVQTYNQLFEEN
PINASGVDAKAILSARLSKSRRLENLIAQLPGEKKNGLFGNLIALSLGLTPNFKSNFDLAED
AKLQLSKDTYDDDLDNLLAQIGDQYADLFLAAKNLSDAILLSDILRVNTEITKAPLSASMIK
RYDEHHQDLTLLKALVRQQLPEKYKEIFFDQSKNGYAGYIDGGASQEEFYKFIKPILEKM
DGTEELLVKLNREDLLRKQRTFDNGSIPHQIHLGELHAILRRQEDFYPFLKDNREKIEKIL
TFRIPYYVGPLARGNSRFAWMTRKSEETITPWNFEEWDKGASAQSFIERMTNFDKNLPNE
KVLPKHSLLYEYFTVYNELTKVKYVTEGMRKPAFLSGEQKKAIVDLLFKTNRKVTVKQLKE
DYFKKIECFDSVEISGVEDRFNASLGTYHDLLKIIKDKDFLDNEENEDILEDIVLTLTLFED
REMIEERLKTYAHLFDDKVMKQLKRRRYTGWGRLSRKLINGIRDKQSGKTILDFLKSDGF
ANRNFMQLIHDDSLTFKEDIQKAQVSGQGDSLHEHIANLAGSPAIKKGILQTVKWDELVK
VMGRHKPENIVIEMARENQTTQKGQKNSRERMKRIEEGIKELGSQILKEHPVENTQLQNE
KLYLYYLQNGRDMYVDQELDINRLSDYDVDHIVPQSFLKDDSIDNKVLTRSDKNRGKSDN
VPSEEWKKMKNYWRQLLNAKLITQRKFDNLTKAERGGLSELDKAGFIKRQLVETRQITK
HVAQILDSRMNTKYDENDKLIREVKVITLKSKLVSDFRKDFQFYKVREINNYHHAHDAYLN
AWGTALIKKYPKLESEFVYGDYKVYDVRKMIAKSEQEIGKATAKYFFYSNIMNFFKTEITL
ANGEIRKRPLIETNGETGEIVWDKGRDFATVRKVLSMPQVNIVKKTEVQTGGFSKESILPK
RNSDKLIARKKDWDPKKYGGFDSPTVAYSVLWAKVEKGKSKKLKSVKELLGITIMERSSF
EKNPIDFLEAKGYKEVKKDLIIKLPKYSLFELENGRKRMLASAGELQKGNELALPSKYVNF
LYLASHYEKLKGSPEDNEQKQLFVEQHKHYLDEIIEQISEFSKRVILADANLDKVLSAYNKH
RDKPIREQAENIIHLFTLTNLGAPAAFKYFDTTIDRKRYTSTKEVLDATLIHQSITGLYETRID
LSQLGGDSGGSGGSGGSTNLSDIIEKETGKQLVIQESILMLPEEVEEVIGNKPESDILVH
TAYDESTDENVMLLTSDAPEYKPWALVIQDSNGENKIKMLSGGSGGSGGSTNLSDII
EKETGKQLVIQESILMLPEEVEEVIGNKPESDILVHTAYDESTDENVMLLTSDAPEYK
PWALVIQDSNGENKIKMLSGGSKRTADGSEFEPKKKRKV
YE1-BE4
(SEQ ID NO: 224)
MKRTADGSEFESPKKKRKVSSETGPVAVDPTLRRRIEPHEFEVFFDPRELRKETCLLY
EINWGGRHSIWRHTSQNTNKHVEVNFIEKFTTERYFCPNTRCSITWFLSYSPCGECSR
AITEFLSRYPHVTLFIYIARLYHHADPENRQGLRDLISSGVTIQIMTEQESGYCWRNFV
NYSPSNEAHWPRYPHLWVRLYVLELYCIILGLPPCLNILRRKQPQLTFFTIALQSCHY
QRLPPHILWATGLKSGGSSGGSSGSETPGTSESATPESSGGSSGGSDKKYSIGLAIGTNS
VGWAVITDEYKVPSKKFKVLGNTDRHSIKKNLIGALLFDSGETAEATRLKRTARRRYTRRK
NRICYLQEIFSNEMAKVDDSFFHRLEESFLVEEDKKHERHPIFGNIVDEVAYHEKYPTIYHL
RKKLVDSTDKADLRLIYLALAHMIKFRGHFLIEGDLNPDNSDVDKLFIQLVQTYNQLFEEN
PINASGVDAKAILSARLSKSRRLENLIAQLPGEKKNGLFGNLIALSLGLTPNFKSNFDLAED
AKLQLSKDTYDDDLDNLLAQIGDQYADLFLAAKNLSDAILLSDILRVNTEITKAPLSASMIK
RYDEHHQDLTLLKALVRQQLPEKYKEIFFDQSKNGYAGYIDGGASQEEFYKFIKPILEKM
DGTEELLVKLNREDLLRKQRTFDNGSIPHQIHLGELHAILRRQEDFYPFLKDNREKIEKIL
TFRIPYYVGPLARGNSRFAWMTRKSEETITPWNFEEWDKGASAQSFIERMTNFDKNLPNE
KVLPKHSLLYEYFTVYNELTKVKYVTEGMRKPAFLSGEQKKAIVDLLFKTNRKVTVKQLKE
DYFKKIECFDSVEISGVEDRFNASLGTYHDLLKIIKDKDFLDNEENEDILEDIVLTLTLFED
REMIEERLKTYAHLFDDKVMKQLKRRRYTGWGRLSRKLINGIRDKQSGKTILDFLKSDGF
ANRNFMQLIHDDSLTFKEDIQKAQVSGQGDSLHEHIANLAGSPAIKKGILQTVKWDELVK
VMGRHKPENIVIEMARENQTTQKGQKNSRERMKRIEEGIKELGSQILKEHPVENTQLQNE
KLYLYYLQNGRDMYVDQELDINRLSDYDVDHIVPQSFLKDDSIDNKVLTRSDKNRGKSDN
VPSEEWKKMKNYWRQLLNAKLITQRKFDNLTKAERGGLSELDKAGFIKRQLVETRQITK
HVAQILDSRMNTKYDENDKLIREVKVITLKSKLVSDFRKDFQFYKVREINNYHHAHDAYLN
AWGTALIKKYPKLESEFVYGDYKVYDVRKMIAKSEQEIGKATAKYFFYSNIMNFFKTEITL
ANGEIRKRPLIETNGETGEIVWDKGRDFATVRKVLSMPQVNIVKKTEVQTGGFSKESILPK
RNSDKLIARKKDWDPKKYGGFDSPTVAYSVLWAKVEKGKSKKLKSVKELLGITIMERSSF
EKNPIDFLEAKGYKEVKKDLIIKLPKYSLFELENGRKRMLASAGELQKGNELALPSKYVNF
LYLASHYEKLKGSPEDNEQKQLFVEQHKHYLDEIIEQISEFSKRVILADANLDKVLSAYNKH
RDKPIREQAENIIHLFTLTNLGAPAAFKYFDTTIDRKRYTSTKEVLDATLIHQSITGLYETRID
LSQLGGDSGGSGGSGGSTNLSDIIEKETGKQLVIQESILMLPEEVEEVIGNKPESDILVH
TAYDESTDENVMLLTSDAPEYKPWALVIQDSNGENKIKMLSGGSGGSGGSTNLSDII
EKETGKQLVIQESILMLPEEVEEVIGNKPESDILVHTAYDESTDENVMLLTSDAPEYK
PWALVIQDSNGENKIKMLSGGSKRTADGSEFEPKKKRKV
YE2-BE4
(SEQ ID NO: 225)
MKRTADGSEFESPKKKRKVSSETGPVAVDPTLRRRIEPHEFEVFFDPRELRKETCLLY
EINWGGRHSIWRHTSQNTNKHVEVNFIEKFTTERYFCPNTRCSITWFLSYSPCGECSR
AITEFLSRYPHVTLFIYIARLYHHADPRNRQGLEDLISSGVTIQIMTEQESGYCWRNFV
NYSPSNEAHWPRYPHLWVRLYVLELYCIILGLPPCLNILRRKQPQLTFFTIALQSCHY
QRLPPHILWATGLKSGGSSGGSSGSETPGTSESATPESSGGSSGGSDKKYSIGLAIGTNS
VGWAVITDEYKVPSKKFKVLGNTDRHSIKKNLIGALLFDSGETAEATRLKRTARRRYTRRK
NRICYLQEIFSNEMAKVDDSFFHRLEESFLVEEDKKHERHPIFGNIVDEVAYHEKYPTIYHL
RKKLVDSTDKADLRLIYLALAHMIKFRGHFLIEGDLNPDNSDVDKLFIQLVQTYNQLFEEN
PINASGVDAKAILSARLSKSRRLENLIAQLPGEKKNGLFGNLIALSLGLTPNFKSNFDLAED
AKLQLSKDTYDDDLDNLLAQIGDQYADLFLAAKNLSDAILLSDILRVNTEITKAPLSASMIK
RYDEHHQDLTLLKALVRQQLPEKYKEIFFDQSKNGYAGYIDGGASQEEFYKFIKPILEKM
DGTEELLVKLNREDLLRKQRTFDNGSIPHQIHLGELHAILRRQEDFYPFLKDNREKIEKIL
TFRIPYYVGPLARGNSRFAWMTRKSEETITPWNFEEWDKGASAQSFIERMTNFDKNLPNE
KVLPKHSLLYEYFTVYNELTKVKYVTEGMRKPAFLSGEQKKAIVDLLFKTNRKVTVKQLKE
DYFKKIECFDSVEISGVEDRFNASLGTYHDLLKIIKDKDFLDNEENEDILEDIVLTLTLFED
REMIEERLKTYAHLFDDKVMKQLKRRRYTGWGRLSRKLINGIRDKQSGKTILDFLKSDGF
ANRNFMQLIHDDSLTFKEDIQKAQVSGQGDSLHEHIANLAGSPAIKKGILQTVKWDELVK
VMGRHKPENIVIEMARENQTTQKGQKNSRERMKRIEEGIKELGSQILKEHPVENTQLQNE
KLYLYYLQNGRDMYVDQELDINRLSDYDVDHIVPQSFLKDDSIDNKVLTRSDKNRGKSDN
VPSEEWKKMKNYWRQLLNAKLITQRKFDNLTKAERGGLSELDKAGFIKRQLVETRQITK
HVAQILDSRMNTKYDENDKLIREVKVITLKSKLVSDFRKDFQFYKVREINNYHHAHDAYLN
AWGTALIKKYPKLESEFVYGDYKVYDVRKMIAKSEQEIGKATAKYFFYSNIMNFFKTEITL
ANGEIRKRPLIETNGETGEIVWDKGRDFATVRKVLSMPQVNIVKKTEVQTGGFSKESILPK
RNSDKLIARKKDWDPKKYGGFDSPTVAYSVLWAKVEKGKSKKLKSVKELLGITIMERSSF
EKNPIDFLEAKGYKEVKKDLIIKLPKYSLFELENGRKRMLASAGELQKGNELALPSKYVNF
LYLASHYEKLKGSPEDNEQKQLFVEQHKHYLDEIIEQISEFSKRVILADANLDKVLSAYNKH
RDKPIREQAENIIHLFTLTNLGAPAAFKYFDTTIDRKRYTSTKEVLDATLIHQSITGLYETRID
LSQLGGDSGGSGGSGGSTNLSDIIEKETGKQLVIQESILMLPEEVEEVIGNKPESDILVH
TAYDESTDENVMLLTSDAPEYKPWALVIQDSNGENKIKMLSGGSGGSGGSTNLSDII
EKETGKQLVIQESILMLPEEVEEVIGNKPESDILVHTAYDESTDENVMLLTSDAPEYK
PWALVIQDSNGENKIKMLSGGSKRTADGSEFEPKKKRKV
YEE-BE4
(SEQ ID NO: 226)
MKRTADGSEFESPKKKRKVSSETGPVAVDPTLRRRIEPHEFEVFFDPRELRKETCLLY
EINWGGRHSIWRHTSQNTNKHVEVNFIEKFTTERYFCPNTRCSITWFLSYSPCGECSR
AITEFLSRYPHVTLFIYIARLYHHADPENRQGLEDLISSGVTIQIMTEQESGYCWRNFV
NYSPSNEAHWPRYPHLWVRLYVLELYCIILGLPPCLNILRRKQPQLTFFTIALQSCHY
QRLPPHILWATGLKSGGSSGGSSGSETPGTSESATPESSGGSSGGSDKKYSIGLAIGTNS
VGWAVITDEYKVPSKKFKVLGNTDRHSIKKNLIGALLFDSGETAEATRLKRTARRRYTRRK
NRICYLQEIFSNEMAKVDDSFFHRLEESFLVEEDKKHERHPIFGNIVDEVAYHEKYPTIYHL
RKKLVDSTDKADLRLIYLALAHMIKFRGHFLIEGDLNPDNSDVDKLFIQLVQTYNQLFEEN
PINASGVDAKAILSARLSKSRRLENLIAQLPGEKKNGLFGNLIALSLGLTPNFKSNFDLAED
AKLQLSKDTYDDDLDNLLAQIGDQYADLFLAAKNLSDAILLSDILRVNTEITKAPLSASMIK
RYDEHHQDLTLLKALVRQQLPEKYKEIFFDQSKNGYAGYIDGGASQEEFYKFIKPILEKM
DGTEELLVKLNREDLLRKQRTFDNGSIPHQIHLGELHAILRRQEDFYPFLKDNREKIEKIL
TFRIPYYVGPLARGNSRFAWMTRKSEETITPWNFEEWDKGASAQSFIERMTNFDKNLPNE
KVLPKHSLLYEYFTVYNELTKVKYVTEGMRKPAFLSGEQKKAIVDLLFKTNRKVTVKQLKE
DYFKKIECFDSVEISGVEDRFNASLGTYHDLLKIIKDKDFLDNEENEDILEDIVLTLTLFED
REMIEERLKTYAHLFDDKVMKQLKRRRYTGWGRLSRKLINGIRDKQSGKTILDFLKSDGF
ANRNFMQLIHDDSLTFKEDIQKAQVSGQGDSLHEHIANLAGSPAIKKGILQTVKWDELVK
VMGRHKPENIVIEMARENQTTQKGQKNSRERMKRIEEGIKELGSQILKEHPVENTQLQNE
KLYLYYLQNGRDMYVDQELDINRLSDYDVDHIVPQSFLKDDSIDNKVLTRSDKNRGKSDN
VPSEEWKKMKNYWRQLLNAKLITQRKFDNLTKAERGGLSELDKAGFIKRQLVETRQITK
HVAQILDSRMNTKYDENDKLIREVKVITLKSKLVSDFRKDFQFYKVREINNYHHAHDAYLN
AWGTALIKKYPKLESEFVYGDYKVYDVRKMIAKSEQEIGKATAKYFFYSNIMNFFKTEITL
ANGEIRKRPLIETNGETGEIVWDKGRDFATVRKVLSMPQVNIVKKTEVQTGGFSKESILPK
RNSDKLIARKKDWDPKKYGGFDSPTVAYSVLWAKVEKGKSKKLKSVKELLGITIMERSSF
EKNPIDFLEAKGYKEVKKDLIIKLPKYSLFELENGRKRMLASAGELQKGNELALPSKYVNF
LYLASHYEKLKGSPEDNEQKQLFVEQHKHYLDEIIEQISEFSKRVILADANLDKVLSAYNKH
RDKPIREQAENIIHLFTLTNLGAPAAFKYFDTTIDRKRYTSTKEVLDATLIHQSITGLYETRID
LSQLGGDSGGSGGSGGSTNLSDIIEKETGKQLVIQESILMLPEEVEEVIGNKPESDILVH
TAYDESTDENVMLLTSDAPEYKPWALVIQDSNGENKIKMLSGGSGGSGGSTNLSDII
EKETGKQLVIQESILMLPEEVEEVIGNKPESDILVHTAYDESTDENVMLLTSDAPEYK
PWALVIQDSNGENKIKMLSGGSKRTADGSEFEPKKKRKV
EE-BE4
(SEQ ID NO: 227)
MKRTADGSEFESPKKKRKVSSETGPVAVDPTLRRRIEPHEFEVFFDPRELRKETCLLY
EINWGGRHSIWRHTSQNTNKHVEVNFIEKFTTERYFCPNTRCSITWFLSWSPCGECSR
AITEFLSRYPHVTLFIYIARLYHHADPENRQGLEDLISSGVTIQIMTEQESGYCWRNFV
NYSPSNEAHWPRYPHLWVRLYVLELYCIILGLPPCLNILRRKQPQLTFFTIALQSCHY
QRLPPHILWATGLKSGGSSGGSSGSETPGTSESATPESSGGSSGGSDKKYSIGLAIGTNS
VGWAVITDEYKVPSKKFKVLGNTDRHSIKKNLIGALLFDSGETAEATRLKRTARRRYTRRK
NRICYLQEIFSNEMAKVDDSFFHRLEESFLVEEDKKHERHPIFGNIVDEVAYHEKYPTIYHL
RKKLVDSTDKADLRLIYLALAHMIKFRGHFLIEGDLNPDNSDVDKLFIQLVQTYNQLFEEN
PINASGVDAKAILSARLSKSRRLENLIAQLPGEKKNGLFGNLIALSLGLTPNFKSNFDLAED
AKLQLSKDTYDDDLDNLLAQIGDQYADLFLAAKNLSDAILLSDILRVNTEITKAPLSASMIK
RYDEHHQDLTLLKALVRQQLPEKYKEIFFDQSKNGYAGYIDGGASQEEFYKFIKPILEKM
DGTEELLVKLNREDLLRKQRTFDNGSIPHQIHLGELHAILRRQEDFYPFLKDNREKIEKIL
TFRIPYYVGPLARGNSRFAWMTRKSEETITPWNFEEWDKGASAQSFIERMTNFDKNLPNE
KVLPKHSLLYEYFTVYNELTKVKYVTEGMRKPAFLSGEQKKAIVDLLFKTNRKVTVKQLKE
DYFKKIECFDSVEISGVEDRFNASLGTYHDLLKIIKDKDFLDNEENEDILEDIVLTLTLFED
REMIEERLKTYAHLFDDKVMKQLKRRRYTGWGRLSRKLINGIRDKQSGKTILDFLKSDGF
ANRNFMQLIHDDSLTFKEDIQKAQVSGQGDSLHEHIANLAGSPAIKKGILQTVKWDELVK
VMGRHKPENIVIEMARENQTTQKGQKNSRERMKRIEEGIKELGSQILKEHPVENTQLQNE
KLYLYYLQNGRDMYVDQELDINRLSDYDVDHIVPQSFLKDDSIDNKVLTRSDKNRGKSDN
VPSEEWKKMKNYWRQLLNAKLITQRKFDNLTKAERGGLSELDKAGFIKRQLVETRQITK
HVAQILDSRMNTKYDENDKLIREVKVITLKSKLVSDFRKDFQFYKVREINNYHHAHDAYLN
AWGTALIKKYPKLESEFVYGDYKVYDVRKMIAKSEQEIGKATAKYFFYSNIMNFFKTEITL
ANGEIRKRPLIETNGETGEIVWDKGRDFATVRKVLSMPQVNIVKKTEVQTGGFSKESILPK
RNSDKLIARKKDWDPKKYGGFDSPTVAYSVLWAKVEKGKSKKLKSVKELLGITIMERSSF
EKNPIDFLEAKGYKEVKKDLIIKLPKYSLFELENGRKRMLASAGELQKGNELALPSKYVNF
LYLASHYEKLKGSPEDNEQKQLFVEQHKHYLDEIIEQISEFSKRVILADANLDKVLSAYNKH
RDKPIREQAENIIHLFTLTNLGAPAAFKYFDTTIDRKRYTSTKEVLDATLIHQSITGLYETRID
LSQLGGDSGGSGGSGGSTNLSDIIEKETGKQLVIQESILMLPEEVEEVIGNKPESDILVH
TAYDESTDENVMLLTSDAPEYKPWALVIQDSNGENKIKMLSGGSGGSGGSTNLSDII
EKETGKQLVIQESILMLPEEVEEVIGNKPESDILVHTAYDESTDENVMLLTSDAPEYK
PWALVIQDSNGENKIKMLSGGSKRTADGSEFEPKKKRKV
R33A-BE4
(SEQ ID NO: 228)
MKRTADGSEFESPKKKRKVSSETGPVAVDPTLRRRIEPHEFEVFFDPRELAKETCLLY
EINWGGRHSIWRHTSQNTNKHVEVNFIEKFTTERYFCPNTRCSITWFLSWSPCGECSR
AITEFLSRYPHVTLFIYIARLYHHADPRNRQGLRDLISSGVTIQIMTEQESGYCWRNFV
NYSPSNEAHWPRYPHLWVRLYVLELYCIILGLPPCLNILRRKQPQLTFFTIALQSCHY
QRLPPHILWATGLKSGGSSGGSSGSETPGTSESATPESSGGSSGGSDKKYSIGLAIGTNS
VGWAVITDEYKVPSKKFKVLGNTDRHSIKKNLIGALLFDSGETAEATRLKRTARRRYTRRK
NRICYLQEIFSNEMAKVDDSFFHRLEESFLVEEDKKHERHPIFGNIVDEVAYHEKYPTIYHL
RKKLVDSTDKADLRLIYLALAHMIKFRGHFLIEGDLNPDNSDVDKLFIQLVQTYNQLFEEN
PINASGVDAKAILSARLSKSRRLENLIAQLPGEKKNGLFGNLIALSLGLTPNFKSNFDLAED
AKLQLSKDTYDDDLDNLLAQIGDQYADLFLAAKNLSDAILLSDILRVNTEITKAPLSASMIK
RYDEHHQDLTLLKALVRQQLPEKYKEIFFDQSKNGYAGYIDGGASQEEFYKFIKPILEKM
DGTEELLVKLNREDLLRKQRTFDNGSIPHQIHLGELHAILRRQEDFYPFLKDNREKIEKIL
TFRIPYYVGPLARGNSRFAWMTRKSEETITPWNFEEWDKGASAQSFIERMTNFDKNLPNE
KVLPKHSLLYEYFTVYNELTKVKYVTEGMRKPAFLSGEQKKAIVDLLFKTNRKVTVKQLKE
DYFKKIECFDSVEISGVEDRFNASLGTYHDLLKIIKDKDFLDNEENEDILEDIVLTLTLFED
REMIEERLKTYAHLFDDKVMKQLKRRRYTGWGRLSRKLINGIRDKQSGKTILDFLKSDGF
ANRNFMQLIHDDSLTFKEDIQKAQVSGQGDSLHEHIANLAGSPAIKKGILQTVKWDELVK
VMGRHKPENIVIEMARENQTTQKGQKNSRERMKRIEEGIKELGSQILKEHPVENTQLQNE
KLYLYYLQNGRDMYVDQELDINRLSDYDVDHIVPQSFLKDDSIDNKVLTRSDKNRGKSDN
VPSEEWKKMKNYWRQLLNAKLITQRKFDNLTKAERGGLSELDKAGFIKRQLVETRQITK
HVAQILDSRMNTKYDENDKLIREVKVITLKSKLVSDFRKDFQFYKVREINNYHHAHDAYLN
AWGTALIKKYPKLESEFVYGDYKVYDVRKMIAKSEQEIGKATAKYFFYSNIMNFFKTEITL
ANGEIRKRPLIETNGETGEIVWDKGRDFATVRKVLSMPQVNIVKKTEVQTGGFSKESILPK
RNSDKLIARKKDWDPKKYGGFDSPTVAYSVLWAKVEKGKSKKLKSVKELLGITIMERSSF
EKNPIDFLEAKGYKEVKKDLIIKLPKYSLFELENGRKRMLASAGELQKGNELALPSKYVNF
LYLASHYEKLKGSPEDNEQKQLFVEQHKHYLDEIIEQISEFSKRVILADANLDKVLSAYNKH
RDKPIREQAENIIHLFTLTNLGAPAAFKYFDTTIDRKRYTSTKEVLDATLIHQSITGLYETRID
LSQLGGDSGGSGGSGGSTNLSDIIEKETGKQLVIQESILMLPEEVEEVIGNKPESDILVH
TAYDESTDENVMLLTSDAPEYKPWALVIQDSNGENKIKMLSGGSGGSGGSTNLSDII
EKETGKQLVIQESILMLPEEVEEVIGNKPESDILVHTAYDESTDENVMLLTSDAPEYK
PWALVIQDSNGENKIKMLSGGSKRTADGSEFEPKKKRKV
R33A + K34A-BE4
(SEQ ID NO: 229)
MKRTADGSEFESPKKKRKVSSETGPVAVDPTLRRRIEPHEFEVFFDPRELAAETCLLY
EINWGGRHSIWRHTSQNTNKHVEVNFIEKFTTERYFCPNTRCSITWFLSWSPCGECSR
AITEFLSRYPHVTLFIYIARLYHHADPRNRQGLRDLISSGVTIQIMTEQESGYCWRNFV
NYSPSNEAHWPRYPHLWVRLYVLELYCIILGLPPCLNILRRKQPQLTFFTIALQSCHY
QRLPPHILWATGLKSGGSSGGSSGSETPGTSESATPESSGGSSGGSDKKYSIGLAIGTNS
VGWAVITDEYKVPSKKFKVLGNTDRHSIKKNLIGALLFDSGETAEATRLKRTARRRYTRRK
NRICYLQEIFSNEMAKVDDSFFHRLEESFLVEEDKKHERHPIFGNIVDEVAYHEKYPTIYHL
RKKLVDSTDKADLRLIYLALAHMIKFRGHFLIEGDLNPDNSDVDKLFIQLVQTYNQLFEEN
PINASGVDAKAILSARLSKSRRLENLIAQLPGEKKNGLFGNLIALSLGLTPNFKSNFDLAED
AKLQLSKDTYDDDLDNLLAQIGDQYADLFLAAKNLSDAILLSDILRVNTEITKAPLSASMIK
RYDEHHQDLTLLKALVRQQLPEKYKEIFFDQSKNGYAGYIDGGASQEEFYKFIKPILEKM
DGTEELLVKLNREDLLRKQRTFDNGSIPHQIHLGELHAILRRQEDFYPFLKDNREKIEKIL
TFRIPYYVGPLARGNSRFAWMTRKSEETITPWNFEEWDKGASAQSFIERMTNFDKNLPNE
KVLPKHSLLYEYFTVYNELTKVKYVTEGMRKPAFLSGEQKKAIVDLLFKTNRKVTVKQLKE
DYFKKIECFDSVEISGVEDRFNASLGTYHDLLKIIKDKDFLDNEENEDILEDIVLTLTLFED
REMIEERLKTYAHLFDDKVMKQLKRRRYTGWGRLSRKLINGIRDKQSGKTILDFLKSDGF
ANRNFMQLIHDDSLTFKEDIQKAQVSGQGDSLHEHIANLAGSPAIKKGILQTVKWDELVK
VMGRHKPENIVIEMARENQTTQKGQKNSRERMKRIEEGIKELGSQILKEHPVENTQLQNE
KLYLYYLQNGRDMYVDQELDINRLSDYDVDHIVPQSFLKDDSIDNKVLTRSDKNRGKSDN
VPSEEWKKMKNYWRQLLNAKLITQRKFDNLTKAERGGLSELDKAGFIKRQLVETRQITK
HVAQILDSRMNTKYDENDKLIREVKVITLKSKLVSDFRKDFQFYKVREINNYHHAHDAYLN
AWGTALIKKYPKLESEFVYGDYKVYDVRKMIAKSEQEIGKATAKYFFYSNIMNFFKTEITL
ANGEIRKRPLIETNGETGEIVWDKGRDFATVRKVLSMPQVNIVKKTEVQTGGFSKESILPK
RNSDKLIARKKDWDPKKYGGFDSPTVAYSVLWAKVEKGKSKKLKSVKELLGITIMERSSF
EKNPIDFLEAKGYKEVKKDLIIKLPKYSLFELENGRKRMLASAGELQKGNELALPSKYVNF
LYLASHYEKLKGSPEDNEQKQLFVEQHKHYLDEIIEQISEFSKRVILADANLDKVLSAYNKH
RDKPIREQAENIIHLFTLTNLGAPAAFKYFDTTIDRKRYTSTKEVLDATLIHQSITGLYETRID
LSQLGGDSGGSGGSGGSTNLSDIIEKETGKQLVIQESILMLPEEVEEVIGNKPESDILVH
TAYDESTDENVMLLTSDAPEYKPWALVIQDSNGENKIKMLSGGSGGSGGSTNLSDII
EKETGKQLVIQESILMLPEEVEEVIGNKPESDILVHTAYDESTDENVMLLTSDAPEYK
PWALVIQDSNGENKIKMLSGGSKRTADGSEFEPKKKRKV
APOBEC3A (A3A)-BE4
(SEQ ID NO: 230)
MKRTADGSEFESPKKKRKVSEASPASGPRHLMDPHIFTSNFNNGIGRHKTYLCYEVE
RLDNGTSVKMDQHRGFLHNQAKNLLCGFYGRHAELRFLDLVPSLQLDPAQIYRVTW
FISWSPCFSWGCAGEVRAFLQENTHVRLRIFAARIYDYDPLYKEALQMLRDAGAQVS
IMTYDEFKHCWDTFVDHQGCPFQPWDGLDEHSQALSGRLRAILQNQGNSGGSSGGS
SGSETPGTSESATPESSGGSSGGSDKKYSIGLAIGTNSVGWAVITDEYKVPSKKFKVLGNT
DRHSIKKNLIGALLFDSGETAEATRLKRTARRRYTRRKNRICYLQEIFSNEMAKVDDSFFHR
LEESFLVEEDKKHERHPIFGNIVDEVAYHEKYPTIYHLRKKLVDSTDKADLRLIYLALAHMI
KFRGHFLIEGDLNPDNSDVDKLFIQLVQTYNQLFEENPINASGVDAKAILSARLSKSRRLEN
LIAQLPGEKKNGLFGNLIALSLGLTPNFKSNFDLAEDAKLQLSKDTYDDDLDNLLAQIGD
QYADLFLAAKNLSDAILLSDILRVNTEITKAPLSASMIKRYDEHHQDLTLLKALVRQQLPEK
YKEIFFDQSKNGYAGYIDGGASQEEFYKFIKPILEKMDGTEELLVKLNREDLLRKQRTFDN
GSIPHQIHLGELHAILRRQEDFYPFLKDNREKIEKILTFRIPYYVGPLARGNSRFAWMTRKS
EETITPWNFEEWDKGASAQSFIERMTNFDKNLPNEKVLPKHSLLYEYFTVYNELTKVKYV
TEGMRKPAFLSGEQKKAIVDLLFKTNRKVTVKQLKEDYFKKIECFDSVEISGVEDRFNASL
GTYHDLLKIIKDKDFLDNEENEDILEDIVLTLTLFEDREMIEERLKTYAHLFDDKVMKQLK
RRRYTGWGRLSRKLINGIRDKQSGKTILDFLKSDGFANRNFMQLIHDDSLTFKEDIQKAQ
VSGQGDSLHEHIANLAGSPAIKKGILQTVKWDELVKVMGRHKPENIVIEMARENQTTQK
GQKNSRERMKRIEEGIKELGSQILKEHPVENTQLQNEKLYLYYLQNGRDMYVDQELDINR
LSDYDVDHIVPQSFLKDDSIDNKVLTRSDKNRGKSDNVPSEEWKKMKNYWRQLLNAKLI
TQRKFDNLTKAERGGLSELDKAGFIKRQLVETRQITKHVAQILDSRMNTKYDENDKLIREV
KVITLKSKLVSDFRKDFQFYKVREINNYHHAHDAYLNAWGTALIKKYPKLESEFVYGDYK
VYDVRKMIAKSEQEIGKATAKYFFYSNIMNFFKTEITLANGEIRKRPLIETNGETGEIVWDK
GRDFATVRKVLSMPQVNIVKKTEVQTGGFSKESILPKRNSDKLIARKKDWDPKKYGGFDS
PTVAYSVLWAKVEKGKSKKLKSVKELLGITIMERSSFEKNPIDFLEAKGYKEVKKDLIIKLP
KYSLFELENGRKRMLASAGELQKGNELALPSKYVNFLYLASHYEKLKGSPEDNEQKQLFV
EQHKHYLDEIIEQISEFSKRVILADANLDKVLSAYNKHRDKPIREQAENIIHLFTLTNLGAPA
AFKYFDTTIDRKRYTSTKEVLDATLIHQSITGLYETRIDLSQLGGDSGGSGGSGGSTNLSDI
IEKETGKQLVIQESILMLPEEVEEVIGNKPESDILVHTAYDESTDENVMLLTSDAPEYK
PWALVIQDSNGENKIKMLSGGSGGSGGSTNLSDIIEKETGKQLVIQESILMLPEEVEEV
IGNKPESDILVHTAYDESTDENVMLLTSDAPEYKPWALVIQDSNGENKIKMLSGGSK
RTADGSEFEPKKKRKV
APOBEC3B (A3B)-BE4
(SEQ ID NO: 231)
MKRTADGSEFESPKKKRKVNPQIRNPMERMYRDTFYDNFENEPILYGRSYTWLCYE
VKIKRGRSNLLWDTGVFRGQVYFKPQYHAEMCFLSWFCGNQLPAYKCFQITWFVS
WTPCPDCVAKLAEFLSEHPNVTLTISAARLYYYWERDYRRALCRLSQAGARVTIMD
YEEFAYCWENFVYNEGQQFMPWYKFDENYAFLHRTLKEILRYLMDPDTFTFNFNND
PLVLRRRQTYLCYEVERLDNGTWVLMDQHMGFLCNEAKNLLCGFYGRHAELRFLD
LVPSLQLDPAQIYRVTWFISWSPCFSWGCAGEVRAFLQENTHVRLRIFAARIYDYDPL
YKEALQMLRDAGAQVSIMTYDEFEYCWDTFVYRQGCPFQPWDGLEEHSQALSGRL
RAILQNQGNSGGSSGGSSGSETPGTSESATPESSGGSSGGSDKKYSIGLAIGTNSVGWAV
ITDEYKVPSKKFKVLGNTDRHSIKKNLIGALLFDSGETAEATRLKRTARRRYTRRKNRICYL
QEIFSNEMAKVDDSFFHRLEESFLVEEDKKHERHPIFGNIVDEVAYHEKYPTIYHLRKKLV
DSTDKADLRLIYLALAHMIKFRGHFLIEGDLNPDNSDVDKLFIQLVQTYNQLFEENPINAS
GVDAKAILSARLSKSRRLENLIAQLPGEKKNGLFGNLIALSLGLTPNFKSNFDLAEDAKLQL
SKDTYDDDLDNLLAQIGDQYADLFLAAKNLSDAILLSDILRVNTEITKAPLSASMIKRYDEH
HQDLTLLKALVRQQLPEKYKEIFFDQSKNGYAGYIDGGASQEEFYKFIKPILEKMDGTEEL
LVKLNREDLLRKQRTFDNGSIPHQIHLGELHAILRRQEDFYPFLKDNREKIEKILTFRIPYY
VGPLARGNSRFAWMTRKSEETITPWNFEEWDKGASAQSFIERMTNFDKNLPNEKVLPKH
SLLYEYFTVYNELTKVKYVTEGMRKPAFLSGEQKKAIVDLLFKTNRKVTVKQLKEDYFKKI
ECFDSVEISGVEDRFNASLGTYHDLLKIIKDKDFLDNEENEDILEDIVLTLTLFEDREMIEE
RLKTYAHLFDDKVMKQLKRRRYTGWGRLSRKLINGIRDKQSGKTILDFLKSDGFANRNFM
QLIHDDSLTFKEDIQKAQVSGQGDSLHEHIANLAGSPAIKKGILQTVKWDELVKVMGRH
KPENIVIEMARENQTTQKGQKNSRERMKRIEEGIKELGSQILKEHPVENTQLQNEKLYLYY
LQNGRDMYVDQELDINRLSDYDVDHIVPQSFLKDDSIDNKVLTRSDKNRGKSDNVPSEEV
VKKMKNYWRQLLNAKLITQRKFDNLTKAERGGLSELDKAGFIKRQLVETRQITKHVAQIL
DSRMNTKYDENDKLIREVKVITLKSKLVSDFRKDFQFYKVREINNYHHAHDAYLNAWGT
ALIKKYPKLESEFVYGDYKVYDVRKMIAKSEQEIGKATAKYFFYSNIMNFFKTEITLANGEI
RKRPLIETNGETGEIVWDKGRDFATVRKVLSMPQVNIVKKTEVQTGGFSKESILPKRNSDK
LIARKKDWDPKKYGGFDSPTVAYSVLWAKVEKGKSKKLKSVKELLGITIMERSSFEKNPI
DFLEAKGYKEVKKDLIIKLPKYSLFELENGRKRMLASAGELQKGNELALPSKYVNFLYLAS
HYEKLKGSPEDNEQKQLFVEQHKHYLDEIIEQISEFSKRVILADANLDKVLSAYNKHRDKP
IREQAENIIHLFTLTNLGAPAAFKYFDTTIDRKRYTSTKEVLDATLIHQSITGLYETRIDLSQL
GGDSGGSGGSGGSTNLSDIIEKETGKQLVIQESILMLPEEVEEVIGNKPESDILVHTAY
DESTDENVMLLTSDAPEYKPWALVIQDSNGENKIKMLSGGSGGSGGSTNLSDIIEKET
GKQLVIQESILMLPEEVEEVIGNKPESDILVHTAYDESTDENVMLLTSDAPEYKPWAL
VIQDSNGENKIKMLSGGSKRTADGSEFEPKKKRKV
APOBEC3G (A3G)-BE4
(SEQ ID NO: 232)
MKRTADGSEFESPKKKRKVKPHFRNTVERMYRDTFSYNFYNRPILSRRNTVWLCYE
VKTKGPSRPPLDAKIFRGQVYSELKYHPEMRFFHWFSKWRKLHRDQEYEVTWYISW
SPCTKCTRDMATFLAEDPKVTLTIFVARLYYFWDPDYQEALRSLCQKRDGPRATMKI
MNYDEFQHCWSKFVYSQRELFEPWNNLPKYYILLHIMLGEILRHSMDPPTFTFNFNN
EPWVRGRHETYLCYEVERMHNDTWVLLNQRRGFLCNQAPHKHGFLEGRHAELCFL
DVIPFWKLDLDQDYRVTCFTSWSPCFSCAQEMAKFISKNKHVSLCIFTARIYDDQGR
CQEGLRTLAEAGAKISIMTYSEFKHCWDTFVDHQGCPFQPWDGLDEHSQDLSGRLR
AILQNQENSGGSSGGSSGSETPGTSESATPESSGGSSGGSDKKYSIGLAIGTNSVGWAVIT
DEYKVPSKKFKVLGNTDRHSIKKNLIGALLFDSGETAEATRLKRTARRRYTRRKNRICYLQE
IFSNEMAKVDDSFFHRLEESFLVEEDKKHERHPIFGNIVDEVAYHEKYPTIYHLRKKLVDS
TDKADLRLIYLALAHMIKFRGHFLIEGDLNPDNSDVDKLFIQLVQTYNQLFEENPINASGV
DAKAILSARLSKSRRLENLIAQLPGEKKNGLFGNLIALSLGLTPNFKSNFDLAEDAKLQLSK
DTYDDDLDNLLAQIGDQYADLFLAAKNLSDAILLSDILRVNTEITKAPLSASMIKRYDEHHQ
DLTLLKALVRQQLPEKYKEIFFDQSKNGYAGYIDGGASQEEFYKFIKPILEKMDGTEELLV
KLNREDLLRKQRTFDNGSIPHQIHLGELHAILRRQEDFYPFLKDNREKIEKILTFRIPYYVG
PLARGNSRFAWMTRKSEETITPWNFEEWDKGASAQSFIERMTNFDKNLPNEKVLPKHSL
LYEYFTVYNELTKVKYVTEGMRKPAFLSGEQKKAIVDLLFKTNRKVTVKQLKEDYFKKIEC
FDSVEISGVEDRFNASLGTYHDLLKIIKDKDFLDNEENEDILEDIVLTLTLFEDREMIEERL
KTYAHLFDDKVMKQLKRRRYTGWGRLSRKLINGIRDKQSGKTILDFLKSDGFANRNFMQ
LIHDDSLTFKEDIQKAQVSGQGDSLHEHIANLAGSPAIKKGILQTVKWDELVKVMGRHK
PENIVIEMARENQTTQKGQKNSRERMKRIEEGIKELGSQILKEHPVENTQLQNEKLYLYYL
QNGRDMYVDQELDINRLSDYDVDHIVPQSFLKDDSIDNKVLTRSDKNRGKSDNVPSEEW
KKMKNYWRQLLNAKLITQRKFDNLTKAERGGLSELDKAGFIKRQLVETRQITKHVAQILD
SRMNTKYDENDKLIREVKVITLKSKLVSDFRKDFQFYKVREINNYHHAHDAYLNAWGTAL
IKKYPKLESEFVYGDYKVYDVRKMIAKSEQEIGKATAKYFFYSNIMNFFKTEITLANGEIRK
RPLIETNGETGEIVWDKGRDFATVRKVLSMPQVNIVKKTEVQTGGFSKESILPKRNSDKLI
ARKKDWDPKKYGGFDSPTVAYSVLWAKVEKGKSKKLKSVKELLGITIMERSSFEKNPIDF
LEAKGYKEVKKDLIIKLPKYSLFELENGRKRMLASAGELQKGNELALPSKYVNFLYLASHY
EKLKGSPEDNEQKQLFVEQHKHYLDEIIEQISEFSKRVILADANLDKVLSAYNKHRDKPIR
EQAENIIHLFTLTNLGAPAAFKYFDTTIDRKRYTSTKEVLDATLIHQSITGLYETRIDLSQLG
GDSGGSGGSGGSTNLSDIIEKETGKQLVIQESILMLPEEVEEVIGNKPESDILVHTAYD
ESTDENVMLLTSDAPEYKPWALVIQDSNGENKIKMLSGGSGGSGGSTNLSDIIEKETG
KQLVIQESILMLPEEVEEVIGNKPESDILVHTAYDESTDENVMLLTSDAPEYKPWALV
IQDSNGENKIKMLSGGSKRTADGSEFEPKKKRKV
AID-BE4
(SEQ ID NO: 233)
MKRTADGSEFESPKKKRKVDSLLMNRRKFLYQFKNVRWAKGRRETYLCYVVKRRD
SATSFSLDFGYLRNKNGCHVELLFLRYISDWDLDPGRCYRVTWFTSWSPCYDCARH
VADFLRGNPNLSLRIFTARLYFCEDRKAEPEGLRRLHRAGVQIAIMTFKDYFYCWNT
FVENHERTFKAWEGLHENSVRLSRQLRRILLPLYEVDDLRDAFRTLGLSGGSSGGSS
GSETPGTSESATPESSGGSSGGSDKKYSIGLAIGTNSVGWAVITDEYKVPSKKFKVLGNTD
RHSIKKNLIGALLFDSGETAEATRLKRTARRRYTRRKNRICYLQEIFSNEMAKVDDSFFHRL
EESFLVEEDKKHERHPIFGNIVDEVAYHEKYPTIYHLRKKLVDSTDKADLRLIYLALAHMIK
FRGHFLIEGDLNPDNSDVDKLFIQLVQTYNQLFEENPINASGVDAKAILSARLSKSRRLENL
IAQLPGEKKNGLFGNLIALSLGLTPNFKSNFDLAEDAKLQLSKDTYDDDLDNLLAQIGDQ
YADLFLAAKNLSDAILLSDILRVNTEITKAPLSASMIKRYDEHHQDLTLLKALVRQQLPEKY
KEIFFDQSKNGYAGYIDGGASQEEFYKFIKPILEKMDGTEELLVKLNREDLLRKQRTFDN
GSIPHQIHLGELHAILRRQEDFYPFLKDNREKIEKILTFRIPYYVGPLARGNSRFAWMTRKS
EETITPWNFEEWDKGASAQSFIERMTNFDKNLPNEKVLPKHSLLYEYFTVYNELTKVKYV
TEGMRKPAFLSGEQKKAIVDLLFKTNRKVTVKQLKEDYFKKIECFDSVEISGVEDRFNASL
GTYHDLLKIIKDKDFLDNEENEDILEDIVLTLTLFEDREMIEERLKTYAHLFDDKVMKQLK
RRRYTGWGRLSRKLINGIRDKQSGKTILDFLKSDGFANRNFMQLIHDDSLTFKEDIQKAQ
VSGQGDSLHEHIANLAGSPAIKKGILQTVKWDELVKVMGRHKPENIVIEMARENQTTQK
GQKNSRERMKRIEEGIKELGSQILKEHPVENTQLQNEKLYLYYLQNGRDMYVDQELDINR
LSDYDVDHIVPQSFLKDDSIDNKVLTRSDKNRGKSDNVPSEEWKKMKNYWRQLLNAKLI
TQRKFDNLTKAERGGLSELDKAGFIKRQLVETRQITKHVAQILDSRMNTKYDENDKLIREV
KVITLKSKLVSDFRKDFQFYKVREINNYHHAHDAYLNAWGTALIKKYPKLESEFVYGDYK
VYDVRKMIAKSEQEIGKATAKYFFYSNIMNFFKTEITLANGEIRKRPLIETNGETGEIVWDK
GRDFATVRKVLSMPQVNIVKKTEVQTGGFSKESILPKRNSDKLIARKKDWDPKKYGGFDS
PTVAYSVLWAKVEKGKSKKLKSVKELLGITIMERSSFEKNPIDFLEAKGYKEVKKDLIIKLP
KYSLFELENGRKRMLASAGELQKGNELALPSKYVNFLYLASHYEKLKGSPEDNEQKQLFV
EQHKHYLDEIIEQISEFSKRVILADANLDKVLSAYNKHRDKPIREQAENIIHLFTLTNLGAPA
AFKYFDTTIDRKRYTSTKEVLDATLIHQSITGLYETRIDLSQLGGDSGGSGGSGGSYNLSDI
IEKETGKQLVIQESILMLPEEVEEVIGNKPESDILVHTAYDESTDENVMLLTSDAPEYK
PWALVIQDSNGENKIKMLSGGSGGSGGSTNLSDIIEKETGKQLVIQESILMLPEEVEEV
IGNKPESDILVHTAYDESTDENVMLLTSDAPEYKPWALVIQDSNGENKIKMLSGGSK
RTADGSEFEPKKKRKV
CDA-BE4
(SEQ ID NO: 234)
MKRTADGSEFESPKKKRKVTDAEYVRIHEKLDIYTFKKQFFNNKKSVSHRCYVLFEL
KRRGERRACFWGYAVNKPQSGTERGIHAEIFSIRKVEEYLRDNPGQFTINWYSSWSP
CADCAEKILEWYNQELRGNGHTLKIWACKLYYEKNARNQIGLWNLRDNGVGLNV
MVSEHYQCCRKIFIQSSHNQLNENRWLEKTLKRAEKRRSELSIMIQVKILHTTKSPAV
SGGSSGGSSGSETPGTSESATPESSGGSSGGSDKKYSIGLAIGTNSVGWAVITDEYKVPSK
KFKVLGNTDRHSIKKNLIGALLFDSGETAEATRLKRTARRRYTRRKNRICYLQEIFSNEMAK
VDDSFFHRLEESFLVEEDKKHERHPIFGNIVDEVAYHEKYPTIYHLRKKLVDSTDKADLRL
IYLALAHMIKFRGHFLIEGDLNPDNSDVDKLFIQLVQTYNQLFEENPINASGVDAKAILSAR
LSKSRRLENLIAQLPGEKKNGLFGNLIALSLGLTPNFKSNFDLAEDAKLQLSKDTYDDDLD
NLLAQIGDQYADLFLAAKNLSDAILLSDILRVNTEITKAPLSASMIKRYDEHHQDLTLLKAL
VRQQLPEKYKEIFFDQSKNGYAGYIDGGASQEEFYKFIKPILEKMDGTEELLVKLNREDLL
RKQRTFDNGSIPHQIHLGELHAILRRQEDFYPFLKDNREKIEKILTFRIPYYVGPLARGNSR
FAWMTRKSEETITPWNFEEWDKGASAQSFIERMTNFDKNLPNEKVLPKHSLLYEYFTVY
NELTKVKYVTEGMRKPAFLSGEQKKAIVDLLFKTNRKVTVKQLKEDYFKKIECFDSVEISG
VEDRFNASLGTYHDLLKIIKDKDFLDNEENEDILEDIVLTLTLFEDREMIEERLKTYAHLFD
DKVMKQLKRRRYTGWGRLSRKLINGIRDKQSGKTILDFLKSDGFANRNFMQLIHDDSLTF
KEDIQKAQVSGQGDSLHEHIANLAGSPAIKKGILQTVKWDELVKVMGRHKPENIVIEMAR
ENQTTQKGQKNSRERMKRIEEGIKELGSQILKEHPVENTQLQNEKLYLYYLQNGRDMYVD
QELDINRLSDYDVDHIVPQSFLKDDSIDNKVLTRSDKNRGKSDNVPSEEWKKMKNYWRQ
LLNAKLITQRKFDNLTKAERGGLSELDKAGFIKRQLVETRQITKHVAQILDSRMNTKYDEN
DKLIREVKVITLKSKLVSDFRKDFQFYKVREINNYHHAHDAYLNAWGTALIKKYPKLESEF
VYGDYKVYDVRKMIAKSEQEIGKATAKYFFYSNIMNFFKTEITLANGEIRKRPLIETNGETG
EIVWDKGRDFATVRKVLSMPQVNIVKKTEVQTGGFSKESILPKRNSDKLIARKKDWDPKK
YGGFDSPTVAYSVLWAKVEKGKSKKLKSVKELLGITIMERSSFEKNPIDFLEAKGYKEVKK
DLIIKLPKYSLFELENGRKRMLASAGELQKGNELALPSKYVNFLYLASHYEKLKGSPEDNE
QKQLFVEQHKHYLDEIIEQISEFSKRVILADANLDKVLSAYNKHRDKPIREQAENIIHLFTLT
NLGAPAAFKYFDTTIDRKRYTSTKEVLDATLIHQSITGLYETRIDLSQLGGDSGGSGGSGGS
TNLSDIIEKETGKQLVIQESILMLPEEVEEVIGNKPESDILVHTAYDESTDENVMLLTS
DAPEYKPWALVIQDSNGENKIKMLSGGSGGSGGSTNLSDIIEKETGKQLVIQESILML
PEEVEEVIGNKPESDILVHTAYDESTDENVMLLTSDAPEYKPWALVIQDSNGENKIK
MLSGGSKRTADGSEFEPKKKRKV
FERNY-BE4
(SEQ ID NO: 235)
MKRTADGSEFESPKKKRKVFERNYDPRELRKETYLLYEIKWGKSGKLWRHWCQNN
RTQHAEVYFLENIFNARRFNPSTHCSITWYLSWSPCAECSQKIVDFLKEHPNVNLEIY
VARLYYHEDERNRQGLRDLVNSGVTIRIMDLPDYNYCWKTFVSDQGGDEDYWPGH
FAPWIKQYSLKLSGGSSGGSSGSETPGTSESATPESSGGSSGGSDKKYSIGLAIGTNSVG
WAVITDEYKVPSKKFKVLGNTDRHSIKKNLIGALLFDSGETAEATRLKRTARRRYTRRKNRI
CYLQEIFSNEMAKVDDSFFHRLEESFLVEEDKKHERHPIFGNIVDEVAYHEKYPTIYHLRK
KLVDSTDKADLRLIYLALAHMIKFRGHFLIEGDLNPDNSDVDKLFIQLVQTYNQLFEENPI
NASGVDAKAILSARLSKSRRLENLIAQLPGEKKNGLFGNLIALSLGLTPNFKSNFDLAEDAK
LQLSKDTYDDDLDNLLAQIGDQYADLFLAAKNLSDAILLSDILRVNTEITKAPLSASMIKRY
DEHHQDLTLLKALVRQQLPEKYKEIFFDQSKNGYAGYIDGGASQEEFYKFIKPILEKMDG
TEELLVKLNREDLLRKQRTFDNGSIPHQIHLGELHAILRRQEDFYPFLKDNREKIEKILTFR
IPYYVGPLARGNSRFAWMTRKSEETITPWNFEEWDKGASAQSFIERMTNFDKNLPNEKVL
PKHSLLYEYFTVYNELTKVKYVTEGMRKPAFLSGEQKKAIVDLLFKTNRKVTVKQLKEDYF
KKIECFDSVEISGVEDRFNASLGTYHDLLKIIKDKDFLDNEENEDILEDIVLTLTLFEDREM
IEERLKTYAHLFDDKVMKQLKRRRYTGWGRLSRKLINGIRDKQSGKTILDFLKSDGFANR
NFMQLIHDDSLTFKEDIQKAQVSGQGDSLHEHIANLAGSPAIKKGILQTVKWDELVKVM
GRHKPENIVIEMARENQTTQKGQKNSRERMKRIEEGIKELGSQILKEHPVENTQLQNEKL
YLYYLQNGRDMYVDQELDINRLSDYDVDHIVPQSFLKDDSIDNKVLTRSDKNRGKSDNVP
SEEWKKMKNYWRQLLNAKLITQRKFDNLTKAERGGLSELDKAGFIKRQLVETRQITKHV
AQILDSRMNTKYDENDKLIREVKVITLKSKLVSDFRKDFQFYKVREINNYHHAHDAYLNAV
VGTALIKKYPKLESEFVYGDYKVYDVRKMIAKSEQEIGKATAKYFFYSNIMNFFKTEITLAN
GEIRKRPLIETNGETGEIVWDKGRDFATVRKVLSMPQVNIVKKTEVQTGGFSKESILPKRN
SDKLIARKKDWDPKKYGGFDSPTVAYSVLWAKVEKGKSKKLKSVKELLGITIMERSSFEK
NPIDFLEAKGYKEVKKDLIIKLPKYSLFELENGRKRMLASAGELQKGNELALPSKYVNFLY
LASHYEKLKGSPEDNEQKQLFVEQHKHYLDEIIEQISEFSKRVILADANLDKVLSAYNKHR
DKPIREQAENIIHLFTLTNLGAPAAFKYFDTTIDRKRYTSTKEVLDATLIHQSITGLYETRIDL
SQLGGDSGGSGGSGGSTNLSDIIEKETGKQLVIQESILMLPEEVEEVIGNKPESDILVHT
AYDESTDENVMLLTSDAPEYKPWALVIQDSNGENKIKMLSGGSGGSGGSTNLSDIIE
KETGKQLVIQESILMLPEEVEEVIGNKPESDILVHTAYDESTDENVMLLTSDAPEYKP
WALVIQDSNGENKIKMLSGGSKRTADGSEFEPKKKRKV
Evolved APOBEC3A (eA3A)-BE4
(SEQ ID NO: 236)
MKRTADGSEFESPKKKRKVEASPASGPRHLMDPHIFTSNFNNGIGRHKTYLCYEVER
LDNGTSVKMDQHRGFLHGQAKNLLCGFYGRHAELRFLDLVPSLQLDPAQIYRVTWF
ISWSPCFSWGCAGEVRAFLQENTHVRLRIFAARIYDYDPLYKEALQMLRDAGAQVSI
MTYDEFKHCWDTFVDHQGCPFQPWDGLDEHSQALSGRLRAILQNQGNSGGSSGGSS
GSETPGTSESATPESSGGSSGGSDKKYSIGLAIGTNSVGWAVITDEYKVPSKKFKVLGNTD
RHSIKKNLIGALLFDSGETAEATRLKRTARRRYTRRKNRICYLQEIFSNEMAKVDDSFFHRL
EESFLVEEDKKHERHPIFGNIVDEVAYHEKYPTIYHLRKKLVDSTDKADLRLIYLALAHMIK
FRGHFLIEGDLNPDNSDVDKLFIQLVQTYNQLFEENPINASGVDAKAILSARLSKSRRLENL
IAQLPGEKKNGLFGNLIALSLGLTPNFKSNFDLAEDAKLQLSKDTYDDDLDNLLAQIGDQ
YADLFLAAKNLSDAILLSDILRVNTEITKAPLSASMIKRYDEHHQDLTLLKALVRQQLPEKY
KEIFFDQSKNGYAGYIDGGASQEEFYKFIKPILEKMDGTEELLVKLNREDLLRKQRTFDN
GSIPHQIHLGELHAILRRQEDFYPFLKDNREKIEKILTFRIPYYVGPLARGNSRFAWMTRKS
EETITPWNFEEWDKGASAQSFIERMTNFDKNLPNEKVLPKHSLLYEYFTVYNELTKVKYV
TEGMRKPAFLSGEQKKAIVDLLFKTNRKVTVKQLKEDYFKKIECFDSVEISGVEDRFNASL
GTYHDLLKIIKDKDFLDNEENEDILEDIVLTLTLFEDREMIEERLKTYAHLFDDKVMKQLK
RRRYTGWGRLSRKLINGIRDKQSGKTILDFLKSDGFANRNFMQLIHDDSLTFKEDIQKAQ
VSGQGDSLHEHIANLAGSPAIKKGILQTVKWDELVKVMGRHKPENIVIEMARENQTTQK
GQKNSRERMKRIEEGIKELGSQILKEHPVENTQLQNEKLYLYYLQNGRDMYVDQELDINR
LSDYDVDHIVPQSFLKDDSIDNKVLTRSDKNRGKSDNVPSEEWKKMKNYWRQLLNAKLI
TQRKFDNLTKAERGGLSELDKAGFIKRQLVETRQITKHVAQILDSRMNTKYDENDKLIREV
KVITLKSKLVSDFRKDFQFYKVREINNYHHAHDAYLNAWGTALIKKYPKLESEFVYGDYK
VYDVRKMIAKSEQEIGKATAKYFFYSNIMNFFKTEITLANGEIRKRPLIETNGETGEIVWDK
GRDFATVRKVLSMPQVNIVKKTEVQTGGFSKESILPKRNSDKLIARKKDWDPKKYGGFDS
PTVAYSVLWAKVEKGKSKKLKSVKELLGITIMERSSFEKNPIDFLEAKGYKEVKKDLIIKLP
KYSLFELENGRKRMLASAGELQKGNELALPSKYVNFLYLASHYEKLKGSPEDNEQKQLFV
EQHKHYLDEIIEQISEFSKRVILADANLDKVLSAYNKHRDKPIREQAENIIHLFTLTNLGAPA
AFKYFDTTIDRKRYTSTKEVLDATLIHQSITGLYETRIDLSQLGGDSGGSGGSGGSTNESD1
IEKETGKQLVIQESILMLPEEVEEVIGNKPESDILVHTAYDESTDENVMLLTSDAPEYK
PWALVIQDSNGENKIKMLSGGSGGSGGSTNLSDIIEKETGKQLVIQESILMLPEEVEEV
IGNKPESDILVHTAYDESTDENVMLLTSDAPEYKPWALVIQDSNGENKIKMLSGGSK
RTADGSEFEPKKKRKV
AALN-BE4
(SEQ ID NO: 237)
MKRTADGSEFESPKKKRKVSSETGPVAVDPTLRRRIEPHEFEVFFDPRELAAETCLLY
EINWGGRHSIWRHTSQNTNKHVEVNFIEKFTTERYFCPNTRCSITWFLSWSPCGECSR
AITEFLSRYPHVTLFIYIARLYHLANPRNRQGLRDLISSGVTIQIMTEQESGYCWRNFV
NYSPSNEAHWPRYPHLWVRLYVLELYCIILGLPPCLNILRRKQPQLTFFTIALQSCHY
QRLPPHILWATGLKSGGSSGGSSGSETPGTSESATPESSGGSSGGSDKKYSIGLAIGTNS
VGWAVITDEYKVPSKKFKVLGNTDRHSIKKNLIGALLFDSGETAEATRLKRTARRRYTRRK
NRICYLQEIFSNEMAKVDDSFFHRLEESFLVEEDKKHERHPIFGNIVDEVAYHEKYPTIYHL
RKKLVDSTDKADLRLIYLALAHMIKFRGHFLIEGDLNPDNSDVDKLFIQLVQTYNQLFEEN
PINASGVDAKAILSARLSKSRRLENLIAQLPGEKKNGLFGNLIALSLGLTPNFKSNFDLAED
AKLQLSKDTYDDDLDNLLAQIGDQYADLFLAAKNLSDAILLSDILRVNTEITKAPLSASMIK
RYDEHHQDLTLLKALVRQQLPEKYKEIFFDQSKNGYAGYIDGGASQEEFYKFIKPILEKM
DGTEELLVKLNREDLLRKQRTFDNGSIPHQIHLGELHAILRRQEDFYPFLKDNREKIEKIL
TFRIPYYVGPLARGNSRFAWMTRKSEETITPWNFEEWDKGASAQSFIERMTNFDKNLPNE
KVLPKHSLLYEYFTVYNELTKVKYVTEGMRKPAFLSGEQKKAIVDLLFKTNRKVTVKQLKE
DYFKKIECFDSVEISGVEDRFNASLGTYHDLLKIIKDKDFLDNEENEDILEDIVLTLTLFED
REMIEERLKTYAHLFDDKVMKQLKRRRYTGWGRLSRKLINGIRDKQSGKTILDFLKSDGF
ANRNFMQLIHDDSLTFKEDIQKAQVSGQGDSLHEHIANLAGSPAIKKGILQTVKWDELVK
VMGRHKPENIVIEMARENQTTQKGQKNSRERMKRIEEGIKELGSQILKEHPVENTQLQNE
KLYLYYLQNGRDMYVDQELDINRLSDYDVDHIVPQSFLKDDSIDNKVLTRSDKNRGKSDN
VPSEEWKKMKNYWRQLLNAKLITQRKFDNLTKAERGGLSELDKAGFIKRQLVETRQITK
HVAQILDSRMNTKYDENDKLIREVKVITLKSKLVSDFRKDFQFYKVREINNYHHAHDAYLN
AWGTALIKKYPKLESEFVYGDYKVYDVRKMIAKSEQEIGKATAKYFFYSNIMNFFKTEITL
ANGEIRKRPLIETNGETGEIVWDKGRDFATVRKVLSMPQVNIVKKTEVQTGGFSKESILPK
RNSDKLIARKKDWDPKKYGGFDSPTVAYSVLWAKVEKGKSKKLKSVKELLGITIMERSSF
EKNPIDFLEAKGYKEVKKDLIIKLPKYSLFELENGRKRMLASAGELQKGNELALPSKYVNF
LYLASHYEKLKGSPEDNEQKQLFVEQHKHYLDEIIEQISEFSKRVILADANLDKVLSAYNKH
RDKPIREQAENIIHLFTLTNLGAPAAFKYFDTTIDRKRYTSTKEVLDATLIHQSITGLYETRID
LSQLGGDSGGSGGSGGSTNLSDIIEKETGKQLVIQESILMLPEEVEEVIGNKPESDILVH
TAYDESTDENVMLLTSDAPEYKPWALVIQDSNGENKIKMLSGGSGGSGGSTNLSDII
EKETGKQLVIQESILMLPEEVEEVIGNKPESDILVHTAYDESTDENVMLLTSDAPEYK
PWALVIQDSNGENKIKMLSGGSKRTADGSEFEPKKKRKV
BE4max, modified with SpCas9-NG
(SEQ ID NO: 238)
MKRTADGSEFESPKKKRKVSSETGPVAVDPTLRRRIEPHEFEVFFDPRELRKETCLLY
EINWGGRHSIWRHTSQNTNKHVEVNFIEKFTTERYFCPNTRCSITWFLSWSPCGECSR
AITEFLSRYPHVTLFIYIARLYHHADPRNRQGLRDLISSGVTIQIMTEQESGYCWRNFV
NYSPSNEAHWPRYPHLWVRLYVLELYCIILGLPPCLNILRRKQPQLTFFTIALQSCHY
QRLPPHILWATGLKSGGSSGGSSGSETPGTSESATPESSGGSSGGSDKKYSIGLAIGTNS
VGWAVITDEYKVPSKKFKVLGNTDRHSIKKNLIGALLFDSGETAEATRLKRTARRRYTRRK
NRICYLQEIFSNEMAKVDDSFFHRLEESFLVEEDKKHERHPIFGNIVDEVAYHEKYPTIYHL
RKKLVDSTDKADLRLIYLALAHMIKFRGHFLIEGDLNPDNSDVDKLFIQLVQTYNQLFEEN
PINASGVDAKAILSARLSKSRRLENLIAQLPGEKKNGLFGNLIALSLGLTPNFKSNFDLAED
AKLQLSKDTYDDDLDNLLAQIGDQYADLFLAAKNLSDAILLSDILRVNTEITKAPLSASMIK
RYDEHHQDLTLLKALVRQQLPEKYKEIFFDQSKNGYAGYIDGGASQEEFYKFIKPILEKM
DGTEELLVKLNREDLLRKQRTFDNGSIPHQIHLGELHAILRRQEDFYPFLKDNREKIEKIL
TFRIPYYVGPLARGNSRFAWMTRKSEETITPWNFEEWDKGASAQSFIERMTNFDKNLPNE
KVLPKHSLLYEYFTVYNELTKVKYVTEGMRKPAFLSGEQKKAIVDLLFKTNRKVTVKQLKE
DYFKKIECFDSVEISGVEDRFNASLGTYHDLLKIIKDKDFLDNEENEDILEDIVLTLTLFED
REMIEERLKTYAHLFDDKVMKQLKRRRYTGWGRLSRKLINGIRDKQSGKTILDFLKSDGF
ANRNFMQLIHDDSLTFKEDIQKAQVSGQGDSLHEHIANLAGSPAIKKGILQTVKWDELVK
VMGRHKPENIVIEMARENQTTQKGQKNSRERMKRIEEGIKELGSQILKEHPVENTQLQNE
KLYLYYLQNGRDMYVDQELDINRLSDYDVDHIVPQSFLKDDSIDNKVLTRSDKNRGKSDN
VPSEEWKKMKNYWRQLLNAKLITQRKFDNLTKAERGGLSELDKAGFIKRQLVETRQITK
HVAQILDSRMNTKYDENDKLIREVKVITLKSKLVSDFRKDFQFYKVREINNYHHAHDAYLN
AWGTALIKKYPKLESEFVYGDYKVYDVRKMIAKSEQEIGKATAKYFFYSNIMNFFKTEITL
ANGEIRKRPLIETNGETGEIVWDKGRDFATVRKVLSMPQVNIVKKTEVQTGGFSKESIRPK
RNSDKLIARKKDWDPKKYGGFVSPTVAYSVLWAKVEKGKSKKLKSVKELLGITIMERSSF
EKNPIDFLEAKGYKEVKKDLIIKLPKYSLFELENGRKRMLASARFLQKGNELALPSKYVNF
LYLASHYEKLKGSPEDNEQKQLFVEQHKHYLDEIIEQISEFSKRVILADANLDKVLSAYNKH
RDKPIREQAENIIHLFTLTNLGAPRAFKYFDTTIDRKVYRSTKEVLDATLIHQSITGLYETRID
LSQLGGDSGGSGGSGGSTNLSDIIEKETGKQLVIQESILMLPEEVEEVIGNKPESDILVH
TAYDESTDENVMLLTSDAPEYKPWALVIQDSNGENKIKMLSGGSGGSGGSTNLSDII
EKETGKQLVIQESILMLPEEVEEVIGNKPESDILVHTAYDESTDENVMLLTSDAPEYK
PWALVIQDSNGENKIKMLSGGSKRTADGSEFEPKKKRKV
YE1-SpCas9-NG base editor (YE1-NG)
(SEQ ID NO: 239)
MKRTADGSEFESPKKKRKVSSETGPVAVDPTLRRRIEPHEFEVFFDPRELRKETCLLY
EINWGGRHSIWRHTSQNTNKHVEVNFIEKFTTERYFCPNTRCSITWFLSYSPCGECSR
AITEFLSRYPHVTLFIYIARLYHHADPENRQGLRDLISSGVTIQIMTEQESGYCWRNFV
NYSPSNEAHWPRYPHLWVRLYVLELYCIILGLPPCLNILRRKQPQLTFFTIALQSCHY
QRLPPHILWATGLKSGGSSGGSSGSETPGTSESATPESSGGSSGGSDKKYSIGLAIGTNS
VGWAVITDEYKVPSKKFKVLGNTDRHSIKKNLIGALLFDSGETAEATRLKRTARRRYTRRK
NRICYLQEIFSNEMAKVDDSFFHRLEESFLVEEDKKHERHPIFGNIVDEVAYHEKYPTIYHL
RKKLVDSTDKADLRLIYLALAHMIKFRGHFLIEGDLNPDNSDVDKLFIQLVQTYNQLFEEN
PINASGVDAKAILSARLSKSRRLENLIAQLPGEKKNGLFGNLIALSLGLTPNFKSNFDLAED
AKLQLSKDTYDDDLDNLLAQIGDQYADLFLAAKNLSDAILLSDILRVNTEITKAPLSASMIK
RYDEHHQDLTLLKALVRQQLPEKYKEIFFDQSKNGYAGYIDGGASQEEFYKFIKPILEKM
DGTEELLVKLNREDLLRKQRTFDNGSIPHQIHLGELHAILRRQEDFYPFLKDNREKIEKIL
TFRIPYYVGPLARGNSRFAWMTRKSEETITPWNFEEWDKGASAQSFIERMTNFDKNLPNE
KVLPKHSLLYEYFTVYNELTKVKYVTEGMRKPAFLSGEQKKAIVDLLFKTNRKVTVKQLKE
DYFKKIECFDSVEISGVEDRFNASLGTYHDLLKIIKDKDFLDNEENEDILEDIVLTLTLFED
REMIEERLKTYAHLFDDKVMKQLKRRRYTGWGRLSRKLINGIRDKQSGKTILDFLKSDGF
ANRNFMQLIHDDSLTFKEDIQKAQVSGQGDSLHEHIANLAGSPAIKKGILQTVKWDELVK
VMGRHKPENIVIEMARENQTTQKGQKNSRERMKRIEEGIKELGSQILKEHPVENTQLQNE
KLYLYYLQNGRDMYVDQELDINRLSDYDVDHIVPQSFLKDDSIDNKVLTRSDKNRGKSDN
VPSEEWKKMKNYWRQLLNAKLITQRKFDNLTKAERGGLSELDKAGFIKRQLVETRQITK
HVAQILDSRMNTKYDENDKLIREVKVITLKSKLVSDFRKDFQFYKVREINNYHHAHDAYLN
AWGTALIKKYPKLESEFVYGDYKVYDVRKMIAKSEQEIGKATAKYFFYSNIMNFFKTEITL
ANGEIRKRPLIETNGETGEIVWDKGRDFATVRKVLSMPQVNIVKKTEVQTGGFSKESIRPK
RNSDKLIARKKDWDPKKYGGFVSPTVAYSVLWAKVEKGKSKKLKSVKELLGITIMERSSF
EKNPIDFLEAKGYKEVKKDLIIKLPKYSLFELENGRKRMLASARFLQKGNELALPSKYVNF
LYLASHYEKLKGSPEDNEQKQLFVEQHKHYLDEIIEQISEFSKRVILADANLDKVLSAYNKH
RDKPIREQAENIIHLFTLTNLGAPRAFKYFDTTIDRKVYRSTKEVLDATLIHQSITGLYETRID
LSQLGGDSGGSGGSGGSTNLSDIIEKETGKQLVIQESILMLPEEVEEVIGNKPESDILVH
TAYDESTDENVMLLTSDAPEYKPWALVIQDSNGENKIKMLSGGSGGSGGSTNLSDII
EKETGKQLVIQESILMLPEEVEEVIGNKPESDILVHTAYDESTDENVMLLTSDAPEYK
PWALVIQDSNGENKIKMLSGGSKRTADGSEFEPKKKRKV
YE2-SpCas9-NG base editor
(SEQ ID NO: 240)
MKRTADGSEFESPKKKRKVSSETGPVAVDPTLRRRIEPHEFEVFFDPRELRKETCLLY
EINWGGRHSIWRHTSQNTNKHVEVNFIEKFTTERYFCPNTRCSITWFLSYSPCGECSR
AITEFLSRYPHVTLFIYIARLYHHADPRNRQGLEDLISSGVTIQIMTEQESGYCWRNFV
NYSPSNEAHWPRYPHLWVRLYVLELYCIILGLPPCLNILRRKQPQLTFFTIALQSCHY
QRLPPHILWATGLKSGGSSGGSSGSETPGTSESATPESSGGSSGGSDKKYSIGLAIGTNS
VGWAVITDEYKVPSKKFKVLGNTDRHSIKKNLIGALLFDSGETAEATRLKRTARRRYTRRK
NRICYLQEIFSNEMAKVDDSFFHRLEESFLVEEDKKHERHPIFGNIVDEVAYHEKYPTIYHL
RKKLVDSTDKADLRLIYLALAHMIKFRGHFLIEGDLNPDNSDVDKLFIQLVQTYNQLFEEN
PINASGVDAKAILSARLSKSRRLENLIAQLPGEKKNGLFGNLIALSLGLTPNFKSNFDLAED
AKLQLSKDTYDDDLDNLLAQIGDQYADLFLAAKNLSDAILLSDILRVNTEITKAPLSASMIK
RYDEHHQDLTLLKALVRQQLPEKYKEIFFDQSKNGYAGYIDGGASQEEFYKFIKPILEKM
DGTEELLVKLNREDLLRKQRTFDNGSIPHQIHLGELHAILRRQEDFYPFLKDNREKIEKIL
TFRIPYYVGPLARGNSRFAWMTRKSEETITPWNFEEWDKGASAQSFIERMTNFDKNLPNE
KVLPKHSLLYEYFTVYNELTKVKYVTEGMRKPAFLSGEQKKAIVDLLFKTNRKVTVKQLKE
DYFKKIECFDSVEISGVEDRFNASLGTYHDLLKIIKDKDFLDNEENEDILEDIVLTLTLFED
REMIEERLKTYAHLFDDKVMKQLKRRRYTGWGRLSRKLINGIRDKQSGKTILDFLKSDGF
ANRNFMQLIHDDSLTFKEDIQKAQVSGQGDSLHEHIANLAGSPAIKKGILQTVKWDELVK
VMGRHKPENIVIEMARENQTTQKGQKNSRERMKRIEEGIKELGSQILKEHPVENTQLQNE
KLYLYYLQNGRDMYVDQELDINRLSDYDVDHIVPQSFLKDDSIDNKVLTRSDKNRGKSDN
VPSEEWKKMKNYWRQLLNAKLITQRKFDNLTKAERGGLSELDKAGFIKRQLVETRQITK
HVAQILDSRMNTKYDENDKLIREVKVITLKSKLVSDFRKDFQFYKVREINNYHHAHDAYLN
AWGTALIKKYPKLESEFVYGDYKVYDVRKMIAKSEQEIGKATAKYFFYSNIMNFFKTEITL
ANGEIRKRPLIETNGETGEIVWDKGRDFATVRKVLSMPQVNIVKKTEVQTGGFSKESIRPK
RNSDKLIARKKDWDPKKYGGFVSPTVAYSVLWAKVEKGKSKKLKSVKELLGITIMERSSF
EKNPIDFLEAKGYKEVKKDLIIKLPKYSLFELENGRKRMLASARFLQKGNELALPSKYVNF
LYLASHYEKLKGSPEDNEQKQLFVEQHKHYLDEIIEQISEFSKRVILADANLDKVLSAYNKH
RDKPIREQAENIIHLFTLTNLGAPRAFKYFDTTIDRKVYRSTKEVLDATLIHQSITGLYETRID
LSQLGGDSGGSGGSGGSTNLSDIIEKETGKQLVIQESILMLPEEVEEVIGNKPESDILVH
TAYDESTDENVMLLTSDAPEYKPWALVIQDSNGENKIKMLSGGSGGSGGSTNLSDII
EKETGKQLVIQESILMLPEEVEEVIGNKPESDILVHTAYDESTDENVMLLTSDAPEYK
PWALVIQDSNGENKIKMLSGGSKRTADGSEFEPKKKRKV
YEE-SpCas9-NG base editor
(SEQ ID NO: 241)
MKRTADGSEFESPKKKRKVSSETGPVAVDPTLRRRIEPHEFEVFFDPRELRKETCLLY
EINWGGRHSIWRHTSQNTNKHVEVNFIEKFTTERYFCPNTRCSITWFLSYSPCGECSR
AITEFLSRYPHVTLFIYIARLYHHADPENRQGLEDLISSGVTIQIMTEQESGYCWRNFV
NYSPSNEAHWPRYPHLWVRLYVLELYCIILGLPPCLNILRRKQPQLTFFTIALQSCHY
QRLPPHILWATGLKSGGSSGGSSGSETPGTSESATPESSGGSSGGSDKKYSIGLAIGTNS
VGWAVITDEYKVPSKKFKVLGNTDRHSIKKNLIGALLFDSGETAEATRLKRTARRRYTRRK
NRICYLQEIFSNEMAKVDDSFFHRLEESFLVEEDKKHERHPIFGNIVDEVAYHEKYPTIYHL
RKKLVDSTDKADLRLIYLALAHMIKFRGHFLIEGDLNPDNSDVDKLFIQLVQTYNQLFEEN
PINASGVDAKAILSARLSKSRRLENLIAQLPGEKKNGLFGNLIALSLGLTPNFKSNFDLAED
AKLQLSKDTYDDDLDNLLAQIGDQYADLFLAAKNLSDAILLSDILRVNTEITKAPLSASMIK
RYDEHHQDLTLLKALVRQQLPEKYKEIFFDQSKNGYAGYIDGGASQEEFYKFIKPILEKM
DGTEELLVKLNREDLLRKQRTFDNGSIPHQIHLGELHAILRRQEDFYPFLKDNREKIEKIL
TFRIPYYVGPLARGNSRFAWMTRKSEETITPWNFEEWDKGASAQSFIERMTNFDKNLPNE
KVLPKHSLLYEYFTVYNELTKVKYVTEGMRKPAFLSGEQKKAIVDLLFKTNRKVTVKQLKE
DYFKKIECFDSVEISGVEDRFNASLGTYHDLLKIIKDKDFLDNEENEDILEDIVLTLTLFED
REMIEERLKTYAHLFDDKVMKQLKRRRYTGWGRLSRKLINGIRDKQSGKTILDFLKSDGF
ANRNFMQLIHDDSLTFKEDIQKAQVSGQGDSLHEHIANLAGSPAIKKGILQTVKWDELVK
VMGRHKPENIVIEMARENQTTQKGQKNSRERMKRIEEGIKELGSQILKEHPVENTQLQNE
KLYLYYLQNGRDMYVDQELDINRLSDYDVDHIVPQSFLKDDSIDNKVLTRSDKNRGKSDN
VPSEEWKKMKNYWRQLLNAKLITQRKFDNLTKAERGGLSELDKAGFIKRQLVETRQITK
HVAQILDSRMNTKYDENDKLIREVKVITLKSKLVSDFRKDFQFYKVREINNYHHAHDAYLN
AWGTALIKKYPKLESEFVYGDYKVYDVRKMIAKSEQEIGKATAKYFFYSNIMNFFKTEITL
ANGEIRKRPLIETNGETGEIVWDKGRDFATVRKVLSMPQVNIVKKTEVQTGGFSKESIRPK
RNSDKLIARKKDWDPKKYGGFVSPTVAYSVLWAKVEKGKSKKLKSVKELLGITIMERSSF
EKNPIDFLEAKGYKEVKKDLIIKLPKYSLFELENGRKRMLASARFLQKGNELALPSKYVNF
LYLASHYEKLKGSPEDNEQKQLFVEQHKHYLDEIIEQISEFSKRVILADANLDKVLSAYNKH
RDKPIREQAENIIHLFTLTNLGAPRAFKYFDTTIDRKVYRSTKEVLDATLIHQSITGLYETRID
LSQLGGDSGGSGGSGGSTNLSDIIEKETGKQLVIQESILMLPEEVEEVIGNKPESDILVH
TAYDESTDENVMLLTSDAPEYKPWALVIQDSNGENKIKMLSGGSGGSGGSTNLSDII
EKETGKQLVIQESILMLPEEVEEVIGNKPESDILVHTAYDESTDENVMLLTSDAPEYK
PWALVIQDSNGENKIKMLSGGSKRTADGSEFEPKKKRKV
EE-SpCas9-NG base editor
(SEQ ID NO: 242)
MKRTADGSEFESPKKKRKVSSETGPVAVDPTLRRRIEPHEFEVFFDPRELRKETCLLY
EINWGGRHSIWRHTSQNTNKHVEVNFIEKFTTERYFCPNTRCSITWFLSWSPCGECSR
AITEFLSRYPHVTLFIYIARLYHHADPENRQGLEDLISSGVTIQIMTEQESGYCWRNFV
NYSPSNEAHWPRYPHLWVRLYVLELYCIILGLPPCLNILRRKQPQLTFFTIALQSCHY
QRLPPHILWATGLKSGGSSGGSSGSETPGTSESATPESSGGSSGGSDKKYSIGLAIGTNS
VGWAVITDEYKVPSKKFKVLGNTDRHSIKKNLIGALLFDSGETAEATRLKRTARRRYTRRK
NRICYLQEIFSNEMAKVDDSFFHRLEESFLVEEDKKHERHPIFGNIVDEVAYHEKYPTIYHL
RKKLVDSTDKADLRLIYLALAHMIKFRGHFLIEGDLNPDNSDVDKLFIQLVQTYNQLFEEN
PINASGVDAKAILSARLSKSRRLENLIAQLPGEKKNGLFGNLIALSLGLTPNFKSNFDLAED
AKLQLSKDTYDDDLDNLLAQIGDQYADLFLAAKNLSDAILLSDILRVNTEITKAPLSASMIK
RYDEHHQDLTLLKALVRQQLPEKYKEIFFDQSKNGYAGYIDGGASQEEFYKFIKPILEKM
DGTEELLVKLNREDLLRKQRTFDNGSIPHQIHLGELHAILRRQEDFYPFLKDNREKIEKIL
TFRIPYYVGPLARGNSRFAWMTRKSEETITPWNFEEWDKGASAQSFIERMTNFDKNLPNE
KVLPKHSLLYEYFTVYNELTKVKYVTEGMRKPAFLSGEQKKAIVDLLFKTNRKVTVKQLKE
DYFKKIECFDSVEISGVEDRFNASLGTYHDLLKIIKDKDFLDNEENEDILEDIVLTLTLFED
REMIEERLKTYAHLFDDKVMKQLKRRRYTGWGRLSRKLINGIRDKQSGKTILDFLKSDGF
ANRNFMQLIHDDSLTFKEDIQKAQVSGQGDSLHEHIANLAGSPAIKKGILQTVKWDELVK
VMGRHKPENIVIEMARENQTTQKGQKNSRERMKRIEEGIKELGSQILKEHPVENTQLQNE
KLYLYYLQNGRDMYVDQELDINRLSDYDVDHIVPQSFLKDDSIDNKVLTRSDKNRGKSDN
VPSEEWKKMKNYWRQLLNAKLITQRKFDNLTKAERGGLSELDKAGFIKRQLVETRQITK
HVAQILDSRMNTKYDENDKLIREVKVITLKSKLVSDFRKDFQFYKVREINNYHHAHDAYLN
AWGTALIKKYPKLESEFVYGDYKVYDVRKMIAKSEQEIGKATAKYFFYSNIMNFFKTEITL
ANGEIRKRPLIETNGETGEIVWDKGRDFATVRKVLSMPQVNIVKKTEVQTGGFSKESIRPK
RNSDKLIARKKDWDPKKYGGFVSPTVAYSVLWAKVEKGKSKKLKSVKELLGITIMERSSF
EKNPIDFLEAKGYKEVKKDLIIKLPKYSLFELENGRKRMLASARFLQKGNELALPSKYVNF
LYLASHYEKLKGSPEDNEQKQLFVEQHKHYLDEIIEQISEFSKRVILADANLDKVLSAYNKH
RDKPIREQAENIIHLFTLTNLGAPRAFKYFDTTIDRKVYRSTKEVLDATLIHQSITGLYETRID
LSQLGGDSGGSGGSGGSTNLSDIIEKETGKQLVIQESILMLPEEVEEVIGNKPESDILVH
TAYDESTDENVMLLTSDAPEYKPWALVIQDSNGENKIKMLSGGSGGSGGSTNLSDII
EKETGKQLVIQESILMLPEEVEEVIGNKPESDILVHTAYDESTDENVMLLTSDAPEYK
PWALVIQDSNGENKIKMLSGGSKRTADGSEFEPKKKRKV
R33A + K34A-SpCas9-NG base editor
(SEQ ID NO: 243)
MKRTADGSEFESPKKKRKVSSETGPVAVDPTLRRRIEPHEFEVFFDPRELAAETCLLY
EINWGGRHSIWRHTSQNTNKHVEVNFIEKFTTERYFCPNTRCSITWFLSWSPCGECSR
AITEFLSRYPHVTLFIYIARLYHHADPRNRQGLRDLISSGVTIQIMTEQESGYCWRNFV
NYSPSNEAHWPRYPHLWVRLYVLELYCIILGLPPCLNILRRKQPQLTFFTIALQSCHY
QRLPPHILWATGLKSGGSSGGSSGSETPGTSESATPESSGGSSGGSDKKYSIGLAIGTNS
VGWAVITDEYKVPSKKFKVLGNTDRHSIKKNLIGALLFDSGETAEATRLKRTARRRYTRRK
NRICYLQEIFSNEMAKVDDSFFHRLEESFLVEEDKKHERHPIFGNIVDEVAYHEKYPTIYHL
RKKLVDSTDKADLRLIYLALAHMIKFRGHFLIEGDLNPDNSDVDKLFIQLVQTYNQLFEEN
PINASGVDAKAILSARLSKSRRLENLIAQLPGEKKNGLFGNLIALSLGLTPNFKSNFDLAED
AKLQLSKDTYDDDLDNLLAQIGDQYADLFLAAKNLSDAILLSDILRVNTEITKAPLSASMIK
RYDEHHQDLTLLKALVRQQLPEKYKEIFFDQSKNGYAGYIDGGASQEEFYKFIKPILEKM
DGTEELLVKLNREDLLRKQRTFDNGSIPHQIHLGELHAILRRQEDFYPFLKDNREKIEKIL
TFRIPYYVGPLARGNSRFAWMTRKSEETITPWNFEEWDKGASAQSFIERMTNFDKNLPNE
KVLPKHSLLYEYFTVYNELTKVKYVTEGMRKPAFLSGEQKKAIVDLLFKTNRKVTVKQLKE
DYFKKIECFDSVEISGVEDRFNASLGTYHDLLKIIKDKDFLDNEENEDILEDIVLTLTLFED
REMIEERLKTYAHLFDDKVMKQLKRRRYTGWGRLSRKLINGIRDKQSGKTILDFLKSDGF
ANRNFMQLIHDDSLTFKEDIQKAQVSGQGDSLHEHIANLAGSPAIKKGILQTVKWDELVK
VMGRHKPENIVIEMARENQTTQKGQKNSRERMKRIEEGIKELGSQILKEHPVENTQLQNE
KLYLYYLQNGRDMYVDQELDINRLSDYDVDHIVPQSFLKDDSIDNKVLTRSDKNRGKSDN
VPSEEWKKMKNYWRQLLNAKLITQRKFDNLTKAERGGLSELDKAGFIKRQLVETRQITK
HVAQILDSRMNTKYDENDKLIREVKVITLKSKLVSDFRKDFQFYKVREINNYHHAHDAYLN
AWGTALIKKYPKLESEFVYGDYKVYDVRKMIAKSEQEIGKATAKYFFYSNIMNFFKTEITL
ANGEIRKRPLIETNGETGEIVWDKGRDFATVRKVLSMPQVNIVKKTEVQTGGFSKESIRPK
RNSDKLIARKKDWDPKKYGGFVSPTVAYSVLWAKVEKGKSKKLKSVKELLGITIMERSSF
EKNPIDFLEAKGYKEVKKDLIIKLPKYSLFELENGRKRMLASARFLQKGNELALPSKYVNF
LYLASHYEKLKGSPEDNEQKQLFVEQHKHYLDEIIEQISEFSKRVILADANLDKVLSAYNKH
RDKPIREQAENIIHLFTLTNLGAPRAFKYFDTTIDRKVYRSTKEVLDATLIHQSITGLYETRID
LSQLGGDSGGSGGSGGSTNLSDIIEKETGKQLVIQESILMLPEEVEEVIGNKPESDILVH
TAYDESTDENVMLLTSDAPEYKPWALVIQDSNGENKIKMLSGGSGGSGGSTNLSDII
EKETGKQLVIQESILMLPEEVEEVIGNKPESDILVHTAYDESTDENVMLLTSDAPEYK
PWALVIQDSNGENKIKMLSGGSKRTADGSEFEPKKKRKV
YE1-CP1028 base editor (YE1-BE4-CP1028, or YE1-CP)
(SEQ ID NO: 244)
MKRTADGSEFESPKKKRKVSSETGPVAVDPTLRRRIEPHEFEVFFDPRELRKETCLLY
EINWGGRHSIWRHTSQNTNKHVEVNFIEKFTTERYFCPNTRCSITWFLSYSPCGECSR
AITEFLSRYPHVTLFIYIARLYHHADPENRQGLRDLISSGVTIQIMTEQESGYCWRNFV
NYSPSNEAHWPRYPHLWVRLYVLELYCIILGLPPCLNILRRKQPQLTFFTIALQSCHY
QRLPPHILWATGLKSGGSSGGSSGSETPGTSESATPESSGGSSGGSEIGKATAKYFFYS
NIMNFFKTEITLANGEIRKRPLIETNGETGEIVWDKGRDFATVRKVLSMPQVNIVKKT
EVQTGGFSKESILPKRNSDKLIARKKDWDPKKYGGFDSPTVAYSVLVVAKVEKGKS
KKLKSVKELLGITIMERSSFEKNPIDFLEAKGYKEVKKDLIIKLPKYSLFELENGRKRM
LASAGELQKGNELALPSKYVNFLYLASHYEKLKGSPEDNEQKQLFVEQHKHYLDEII
EQISEFSKRVILADANLDKVLSAYNKHRDKPIREQAENIIHLFTLTNLGAPAAFKYFDT
TIDRKRYTSTKEVLDATLIHQSITGLYETRIDLSQLGGDGGSGGSGGSGGSGGSGGSG
GMDKKYSIGLAIGTNSVGWAVITDEYKVPSKKFKVLGNTDRHSIKKNLIGALLFDSGETAE
ATRLKRTARRRYTRRKNRICYLQEIFSNEMAKVDDSFFHRLEESFLVEEDKKHERHPIFGNI
VDEVAYHEKYPTIYHLRKKLVDSTDKADLRLIYLALAHMIKFRGHFLIEGDLNPDNSDVDK
LFIQLVQTYNQLFEENPINASGVDAKAILSARLSKSRRLENLIAQLPGEKKNGLFGNLIALSL
GLTPNFKSNFDLAEDAKLQLSKDTYDDDLDNLLAQIGDQYADLFLAAKNLSDAILLSDILR
VNTEITKAPLSASMIKRYDEHHQDLTLLKALVRQQLPEKYKEIFFDQSKNGYAGYIDGGAS
QEEFYKFIKPILEKMDGTEELLVKLNREDLLRKQRTFDNGSIPHQIHLGELHAILRRQEDF
YPFLKDNREKIEKILTFRIPYYVGPLARGNSRFAWMTRKSEETITPWNFEEWDKGASAQSF
IERMTNFDKNLPNEKVLPKHSLLYEYFTVYNELTKVKYVTEGMRKPAFLSGEQKKAIVDLL
FKTNRKVTVKQLKEDYFKKIECFDSVEISGVEDRFNASLGTYHDLLKIIKDKDFLDNEENE
DILEDIVLTLTLFEDREMIEERLKTYAHLFDDKVMKQLKRRRYTGWGRLSRKLINGIRDKQ
SGKTILDFLKSDGFANRNFMQLIHDDSLTFKEDIQKAQVSGQGDSLHEHIANLAGSPAIKK
GILQTVKWDELVKVMGRHKPENIVIEMARENQTTQKGQKNSRERMKRIEEGIKELGSQIL
KEHPVENTQLQNEKLYLYYLQNGRDMYVDQELDINRLSDYDVDHIVPQSFLKDDSIDNKV
LTRSDKNRGKSDNVPSEEWKKMKNYWRQLLNAKLITQRKFDNLTKAERGGLSELDKAG
FIKRQLVETRQITKHVAQILDSRMNTKYDENDKLIREVKVITLKSKLVSDFRKDFQFYKVRE
INNYHHAHDAYLNAWGTALIKKYPKLESEFVYGDYKVYDVRKMIAKSEQSGGSGGSGGS
TNLSDIIEKETGKQLVIQESILMLPEEVEEVIGNKPESDILVHTAYDESTDENVMLLTS
DAPEYKPWALVIQDSNGENKIKMLSGGSGGSGGSTNLSDIIEKETGKQLVIQESILML
PEEVEEVIGNKPESDILVHTAYDESTDENVMLLTSDAPEYKPWALVIQDSNGENKIK
MLSGGSKRTADGSEFEPKKKRKV
YE2-CP1028 base editor (YE2-BE4-CP1028)
(SEQ ID NO: 245)
MKRTADGSEFESPKKKRKVSSETGPVAVDPTLRRRIEPHEFEVFFDPRELRKETCLLY
EINWGGRHSIWRHTSQNTNKHVEVNFIEKFTTERYFCPNTRCSITWFLSYSPCGECSR
AITEFLSRYPHVTLFIYIARLYHHADPRNRQGLEDLISSGVTIQIMTEQESGYCWRNFV
NYSPSNEAHWPRYPHLWVRLYVLELYCIILGLPPCLNILRRKQPQLTFFTIALQSCHY
QRLPPHILWATGLKSGGSSGGSSGSETPGTSESATPESSGGSSGGSEIGKATAKYFFYS
NIMNFFKTEITLANGEIRKRPLIETNGETGEIVWDKGRDFATVRKVLSMPQVNIVKKT
EVQTGGFSKESILPKRNSDKLIARKKDWDPKKYGGFDSPTVAYSVLVVAKVEKGKS
KKLKSVKELLGITIMERSSFEKNPIDFLEAKGYKEVKKDLIIKLPKYSLFELENGRKRM
LASAGELQKGNELALPSKYVNFLYLASHYEKLKGSPEDNEQKQLFVEQHKHYLDEII
EQISEFSKRVILADANLDKVLSAYNKHRDKPIREQAENIIHLFTLTNLGAPAAFKYFDT
TIDRKRYTSTKEVLDATLIHQSITGLYETRIDLSQLGGDGGSGGSGGSGGSGGSGGSG
GMDKKYSIGLAIGTNSVGWAVITDEYKVPSKKFKVLGNTDRHSIKKNLIGALLFDSGETAE
ATRLKRTARRRYTRRKNRICYLQEIFSNEMAKVDDSFFHRLEESFLVEEDKKHERHPIFGNI
VDEVAYHEKYPTIYHLRKKLVDSTDKADLRLIYLALAHMIKFRGHFLIEGDLNPDNSDVDK
LFIQLVQTYNQLFEENPINASGVDAKAILSARLSKSRRLENLIAQLPGEKKNGLFGNLIALSL
GLTPNFKSNFDLAEDAKLQLSKDTYDDDLDNLLAQIGDQYADLFLAAKNLSDAILLSDILR
VNTEITKAPLSASMIKRYDEHHQDLTLLKALVRQQLPEKYKEIFFDQSKNGYAGYIDGGAS
QEEFYKFIKPILEKMDGTEELLVKLNREDLLRKQRTFDNGSIPHQIHLGELHAILRRQEDF
YPFLKDNREKIEKILTFRIPYYVGPLARGNSRFAWMTRKSEETITPWNFEEWDKGASAQSF
IERMTNFDKNLPNEKVLPKHSLLYEYFTVYNELTKVKYVTEGMRKPAFLSGEQKKAIVDLL
FKTNRKVTVKQLKEDYFKKIECFDSVEISGVEDRFNASLGTYHDLLKIIKDKDFLDNEENE
DILEDIVLTLTLFEDREMIEERLKTYAHLFDDKVMKQLKRRRYTGWGRLSRKLINGIRDKQ
SGKTILDFLKSDGFANRNFMQLIHDDSLTFKEDIQKAQVSGQGDSLHEHIANLAGSPAIKK
GILQTVKWDELVKVMGRHKPENIVIEMARENQTTQKGQKNSRERMKRIEEGIKELGSQIL
KEHPVENTQLQNEKLYLYYLQNGRDMYVDQELDINRLSDYDVDHIVPQSFLKDDSIDNKV
LTRSDKNRGKSDNVPSEEWKKMKNYWRQLLNAKLITQRKFDNLTKAERGGLSELDKAG
FIKRQLVETRQITKHVAQILDSRMNTKYDENDKLIREVKVITLKSKLVSDFRKDFQFYKVRE
INNYHHAHDAYLNAWGTALIKKYPKLESEFVYGDYKVYDVRKMIAKSEQSGGSGGSGGS
TNLSDIIEKETGKQLVIQESILMLPEEVEEVIGNKPESDILVHTAYDESTDENVMLLTS
DAPEYKPWALVIQDSNGENKIKMLSGGSGGSGGSTNLSDIIEKETGKQLVIQESILML
PEEVEEVIGNKPESDILVHTAYDESTDENVMLLTSDAPEYKPWALVIQDSNGENKIK
MLSGGSKRTADGSEFEPKKKRKV
YEE-CP1028 base editor (YEE-BE4-CP1028)
(SEQ ID NO: 246)
MKRTADGSEFESPKKKRKVSSETGPVAVDPTLRRRIEPHEFEVFFDPRELRKETCLLY
EINWGGRHSIWRHTSQNTNKHVEVNFIEKFTTERYFCPNTRCSITWFLSYSPCGECSR
AITEFLSRYPHVTLFIYIARLYHHADPENRQGLEDLISSGVTIQIMTEQESGYCWRNFV
NYSPSNEAHWPRYPHLWVRLYVLELYCIILGLPPCLNILRRKQPQLTFFTIALQSCHY
QRLPPHILWATGLKSGGSSGGSSGSETPGTSESATPESSGGSSGGSEIGKATAKYFFYS
NIMNFFKTEITLANGEIRKRPLIETNGETGEIVWDKGRDFATVRKVLSMPQVNIVKKT
EVQTGGFSKESILPKRNSDKLIARKKDWDPKKYGGFDSPTVAYSVLVVAKVEKGKS
KKLKSVKELLGITIMERSSFEKNPIDFLEAKGYKEVKKDLIIKLPKYSLFELENGRKRM
LASAGELQKGNELALPSKYVNFLYLASHYEKLKGSPEDNEQKQLFVEQHKHYLDEII
EQISEFSKRVILADANLDKVLSAYNKHRDKPIREQAENIIHLFTLTNLGAPAAFKYFDT
TIDRKRYTSTKEVLDATLIHQSITGLYETRIDLSQLGGDGGSGGSGGSGGSGGSGGSG
GMDKKYSIGLAIGTNSVGWAVITDEYKVPSKKFKVLGNTDRHSIKKNLIGALLFDSGETAE
ATRLKRTARRRYTRRKNRICYLQEIFSNEMAKVDDSFFHRLEESFLVEEDKKHERHPIFGNI
VDEVAYHEKYPTIYHLRKKLVDSTDKADLRLIYLALAHMIKFRGHFLIEGDLNPDNSDVDK
LFIQLVQTYNQLFEENPINASGVDAKAILSARLSKSRRLENLIAQLPGEKKNGLFGNLIALSL
GLTPNFKSNFDLAEDAKLQLSKDTYDDDLDNLLAQIGDQYADLFLAAKNLSDAILLSDILR
VNTEITKAPLSASMIKRYDEHHQDLTLLKALVRQQLPEKYKEIFFDQSKNGYAGYIDGGAS
QEEFYKFIKPILEKMDGTEELLVKLNREDLLRKQRTFDNGSIPHQIHLGELHAILRRQEDF
YPFLKDNREKIEKILTFRIPYYVGPLARGNSRFAWMTRKSEETITPWNFEEWDKGASAQSF
IERMTNFDKNLPNEKVLPKHSLLYEYFTVYNELTKVKYVTEGMRKPAFLSGEQKKAIVDLL
FKTNRKVTVKQLKEDYFKKIECFDSVEISGVEDRFNASLGTYHDLLKIIKDKDFLDNEENE
DILEDIVLTLTLFEDREMIEERLKTYAHLFDDKVMKQLKRRRYTGWGRLSRKLINGIRDKQ
SGKTILDFLKSDGFANRNFMQLIHDDSLTFKEDIQKAQVSGQGDSLHEHIANLAGSPAIKK
GILQTVKWDELVKVMGRHKPENIVIEMARENQTTQKGQKNSRERMKRIEEGIKELGSQIL
KEHPVENTQLQNEKLYLYYLQNGRDMYVDQELDINRLSDYDVDHIVPQSFLKDDSIDNKV
LTRSDKNRGKSDNVPSEEWKKMKNYWRQLLNAKLITQRKFDNLTKAERGGLSELDKAG
FIKRQLVETRQITKHVAQILDSRMNTKYDENDKLIREVKVITLKSKLVSDFRKDFQFYKVRE
INNYHHAHDAYLNAWGTALIKKYPKLESEFVYGDYKVYDVRKMIAKSEQSGGSGGSGGS
TNLSDIIEKETGKQLVIQESILMLPEEVEEVIGNKPESDILVHTAYDESTDENVMLLTS
DAPEYKPWALVIQDSNGENKIKMLSGGSGGSGGSTNLSDIIEKETGKQLVIQESILML
PEEVEEVIGNKPESDILVHTAYDESTDENVMLLTSDAPEYKPWALVIQDSNGENKIK
MLSGGSKRTADGSEFEPKKKRKV
EE-CP1028 base editor (EE-BE4-CP1028)
(SEQ ID NO: 247)
MKRTADGSEFESPKKKRKVSSETGPVAVDPTLRRRIEPHEFEVFFDPRELRKETCLLY
EINWGGRHSIWRHTSQNTNKHVEVNFIEKFTTERYFCPNTRCSITWFLSWSPCGECSR
AITEFLSRYPHVTLFIYIARLYHHADPENRQGLEDLISSGVTIQIMTEQESGYCWRNFV
NYSPSNEAHWPRYPHLWVRLYVLELYCIILGLPPCLNILRRKQPQLTFFTIALQSCHY
QRLPPHILWATGLKSGGSSGGSSGSETPGTSESATPESSGGSSGGSEIGKATAKYFFYS
NIMNFFKTEITLANGEIRKRPLIETNGETGEIVWDKGRDFATVRKVLSMPQVNIVKKT
EVQTGGFSKESILPKRNSDKLIARKKDWDPKKYGGFDSPTVAYSVLVVAKVEKGKS
KKLKSVKELLGITIMERSSFEKNPIDFLEAKGYKEVKKDLIIKLPKYSLFELENGRKRM
LASAGELQKGNELALPSKYVNFLYLASHYEKLKGSPEDNEQKQLFVEQHKHYLDEII
EQISEFSKRVILADANLDKVLSAYNKHRDKPIREQAENIIHLFTLTNLGAPAAFKYFDT
TIDRKRYTSTKEVLDATLIHQSITGLYETRIDLSQLGGDGGSGGSGGSGGSGGSGGSG
GMDKKYSIGLAIGTNSVGWAVITDEYKVPSKKFKVLGNTDRHSIKKNLIGALLFDSGETAE
ATRLKRTARRRYTRRKNRICYLQEIFSNEMAKVDDSFFHRLEESFLVEEDKKHERHPIFGNI
VDEVAYHEKYPTIYHLRKKLVDSTDKADLRLIYLALAHMIKFRGHFLIEGDLNPDNSDVDK
LFIQLVQTYNQLFEENPINASGVDAKAILSARLSKSRRLENLIAQLPGEKKNGLFGNLIALSL
GLTPNFKSNFDLAEDAKLQLSKDTYDDDLDNLLAQIGDQYADLFLAAKNLSDAILLSDILR
VNTEITKAPLSASMIKRYDEHHQDLTLLKALVRQQLPEKYKEIFFDQSKNGYAGYIDGGAS
QEEFYKFIKPILEKMDGTEELLVKLNREDLLRKQRTFDNGSIPHQIHLGELHAILRRQEDF
YPFLKDNREKIEKILTFRIPYYVGPLARGNSRFAWMTRKSEETITPWNFEEWDKGASAQSF
IERMTNFDKNLPNEKVLPKHSLLYEYFTVYNELTKVKYVTEGMRKPAFLSGEQKKAIVDLL
FKTNRKVTVKQLKEDYFKKIECFDSVEISGVEDRFNASLGTYHDLLKIIKDKDFLDNEENE
DILEDIVLTLTLFEDREMIEERLKTYAHLFDDKVMKQLKRRRYTGWGRLSRKLINGIRDKQ
SGKTILDFLKSDGFANRNFMQLIHDDSLTFKEDIQKAQVSGQGDSLHEHIANLAGSPAIKK
GILQTVKWDELVKVMGRHKPENIVIEMARENQTTQKGQKNSRERMKRIEEGIKELGSQIL
KEHPVENTQLQNEKLYLYYLQNGRDMYVDQELDINRLSDYDVDHIVPQSFLKDDSIDNKV
LTRSDKNRGKSDNVPSEEWKKMKNYWRQLLNAKLITQRKFDNLTKAERGGLSELDKAG
FIKRQLVETRQITKHVAQILDSRMNTKYDENDKLIREVKVITLKSKLVSDFRKDFQFYKVRE
INNYHHAHDAYLNAWGTALIKKYPKLESEFVYGDYKVYDVRKMIAKSEQSGGSGGSGGS
TNLSDIIEKETGKQLVIQESILMLPEEVEEVIGNKPESDILVHTAYDESTDENVMLLTS
DAPEYKPWALVIQDSNGENKIKMLSGGSGGSGGSTNLSDIIEKETGKQLVIQESILML
PEEVEEVIGNKPESDILVHTAYDESTDENVMLLTSDAPEYKPWALVIQDSNGENKIK
MLSGGSKRTADGSEFEPKKKRKV
R33A + K34A-CP1028 base editor (R33A + K34A-BE4-CP1028)
(SEQ ID NO: 248)
MKRTADGSEFESPKKKRKVSSETGPVAVDPTLRRRIEPHEFEVFFDPRELAAETCLLY
EINWGGRHSIWRHTSQNTNKHVEVNFIEKFTTERYFCPNTRCSITWFLSWSPCGECSR
AITEFLSRYPHVTLFIYIARLYHHADPRNRQGLRDLISSGVTIQIMTEQESGYCWRNFV
NYSPSNEAHWPRYPHLWVRLYVLELYCIILGLPPCLNILRRKQPQLTFFTIALQSCHY
QRLPPHILWATGLKSGGSSGGSSGSETPGTSESATPESSGGSSGGSEIGKATAKYFFYS
NIMNFFKTEITLANGEIRKRPLIETNGETGEIVWDKGRDFATVRKVLSMPQVNIVKKT
EVQTGGFSKESILPKRNSDKLIARKKDWDPKKYGGFDSPTVAYSVLVVAKVEKGKS
KKLKSVKELLGITIMERSSFEKNPIDFLEAKGYKEVKKDLIIKLPKYSLFELENGRKRM
LASAGELQKGNELALPSKYVNFLYLASHYEKLKGSPEDNEQKQLFVEQHKHYLDEII
EQISEFSKRVILADANLDKVLSAYNKHRDKPIREQAENIIHLFTLTNLGAPAAFKYFDT
TIDRKRYTSTKEVLDATLIHQSITGLYETRIDLSQLGGDGGSGGSGGSGGSGGSGGSG
GMDKKYSIGLAIGTNSVGWAVITDEYKVPSKKFKVLGNTDRHSIKKNLIGALLFDSGETAE
ATRLKRTARRRYTRRKNRICYLQEIFSNEMAKVDDSFFHRLEESFLVEEDKKHERHPIFGNI
VDEVAYHEKYPTIYHLRKKLVDSTDKADLRLIYLALAHMIKFRGHFLIEGDLNPDNSDVDK
LFIQLVQTYNQLFEENPINASGVDAKAILSARLSKSRRLENLIAQLPGEKKNGLFGNLIALSL
GLTPNFKSNFDLAEDAKLQLSKDTYDDDLDNLLAQIGDQYADLFLAAKNLSDAILLSDILR
VNTEITKAPLSASMIKRYDEHHQDLTLLKALVRQQLPEKYKEIFFDQSKNGYAGYIDGGAS
QEEFYKFIKPILEKMDGTEELLVKLNREDLLRKQRTFDNGSIPHQIHLGELHAILRRQEDF
YPFLKDNREKIEKILTFRIPYYVGPLARGNSRFAWMTRKSEETITPWNFEEWDKGASAQSF
IERMTNFDKNLPNEKVLPKHSLLYEYFTVYNELTKVKYVTEGMRKPAFLSGEQKKAIVDLL
FKTNRKVTVKQLKEDYFKKIECFDSVEISGVEDRFNASLGTYHDLLKIIKDKDFLDNEENE
DILEDIVLTLTLFEDREMIEERLKTYAHLFDDKVMKQLKRRRYTGWGRLSRKLINGIRDKQ
SGKTILDFLKSDGFANRNFMQLIHDDSLTFKEDIQKAQVSGQGDSLHEHIANLAGSPAIKK
GILQTVKWDELVKVMGRHKPENIVIEMARENQTTQKGQKNSRERMKRIEEGIKELGSQIL
KEHPVENTQLQNEKLYLYYLQNGRDMYVDQELDINRLSDYDVDHIVPQSFLKDDSIDNKV
LTRSDKNRGKSDNVPSEEWKKMKNYWRQLLNAKLITQRKFDNLTKAERGGLSELDKAG
FIKRQLVETRQITKHVAQILDSRMNTKYDENDKLIREVKVITLKSKLVSDFRKDFQFYKVRE
INNYHHAHDAYLNAWGTALIKKYPKLESEFVYGDYKVYDVRKMIAKSEQSGGSGGSGGS
TNLSDIIEKETGKQLVIQESILMLPEEVEEVIGNKPESDILVHTAYDESTDENVMLLTS
DAPEYKPWALVIQDSNGENKIKMLSGGSGGSGGSTNLSDIIEKETGKQLVIQESILML
PEEVEEVIGNKPESDILVHTAYDESTDENVMLLTSDAPEYKPWALVIQDSNGENKIK
MLSGGSKRTADGSEFEPKKKRKV

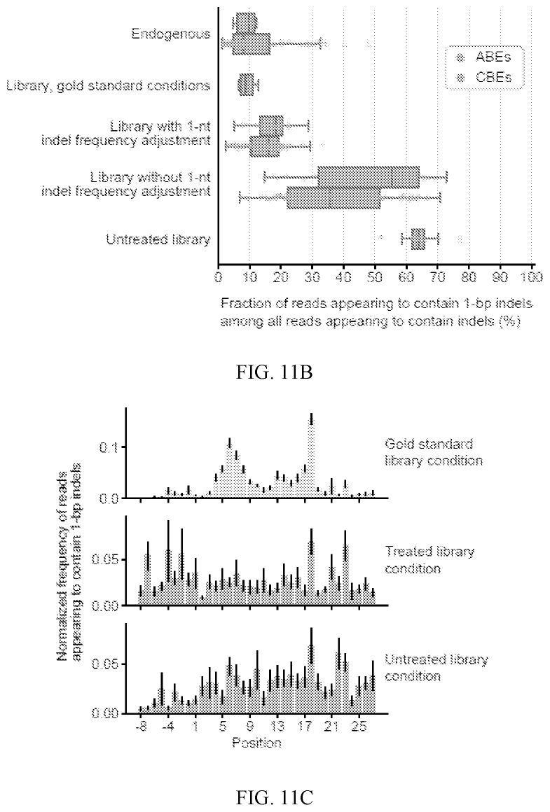

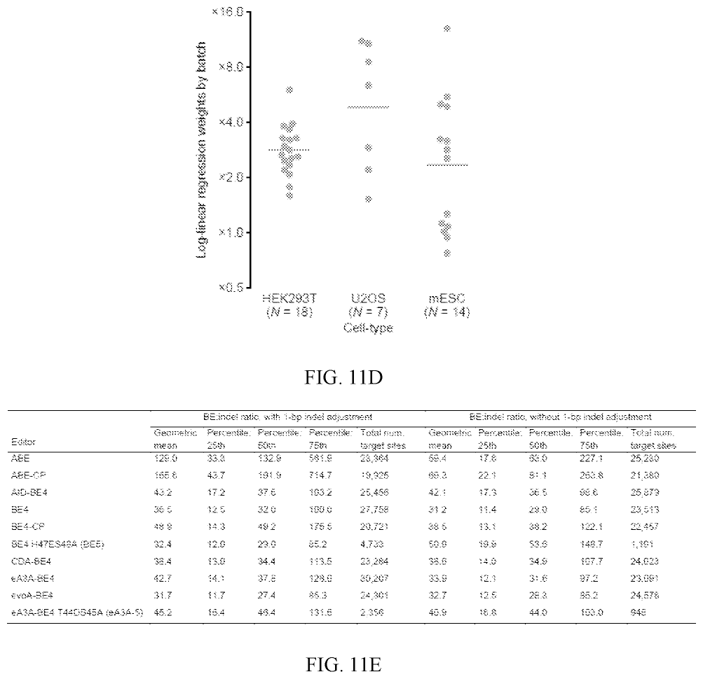

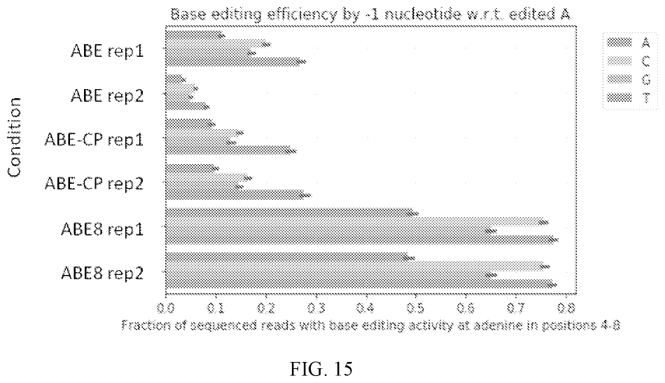

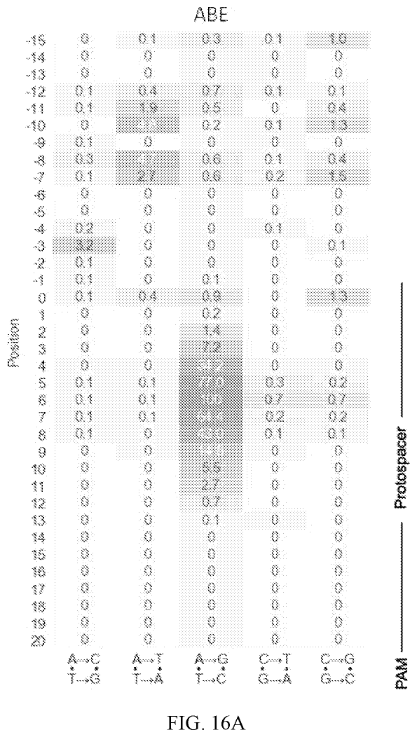

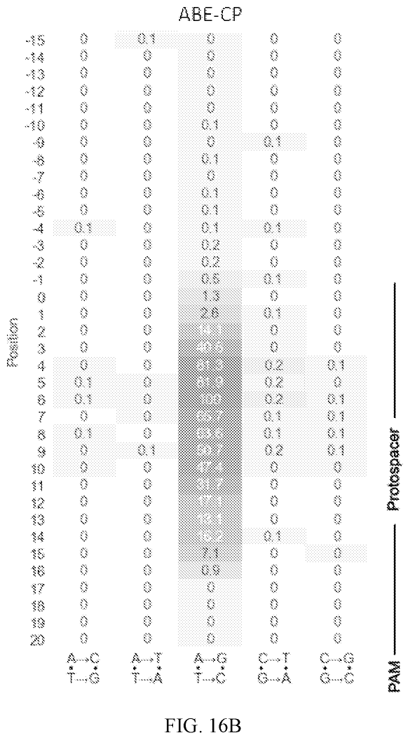

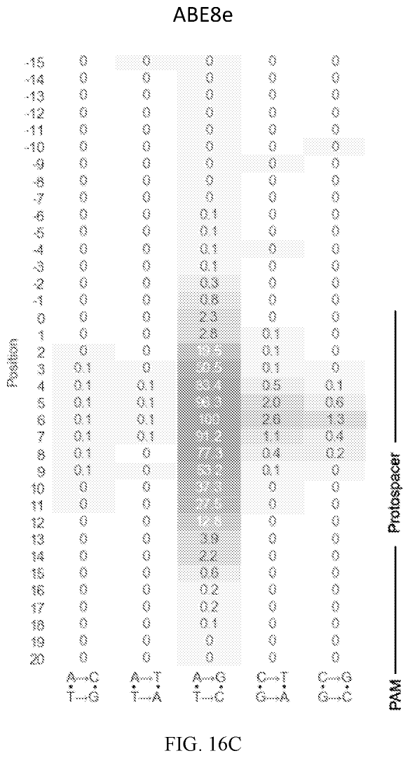

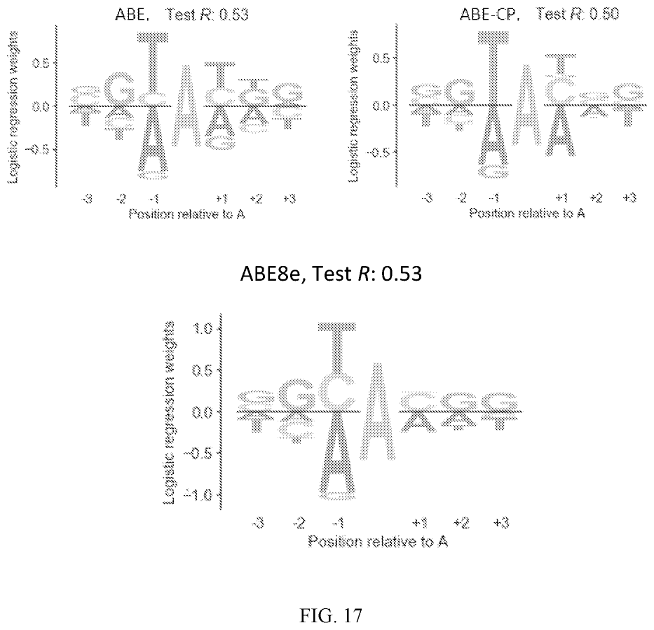

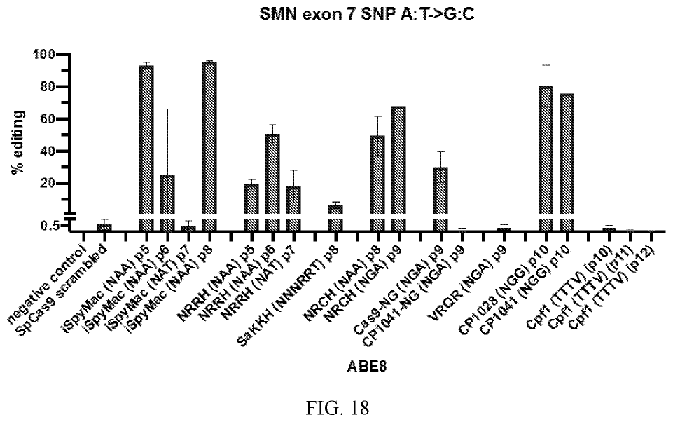

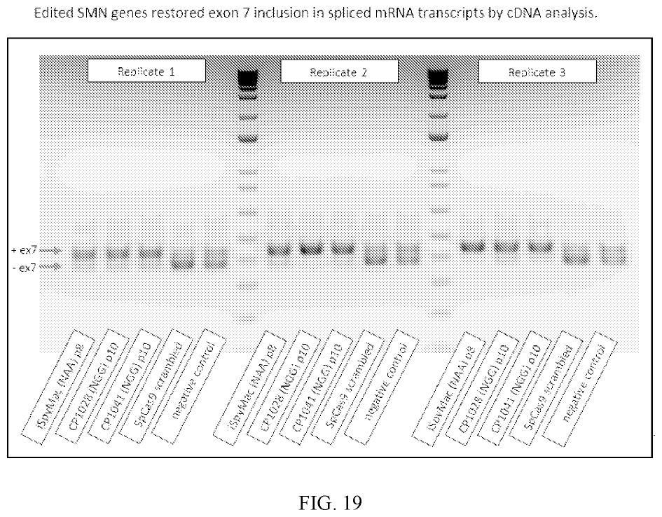

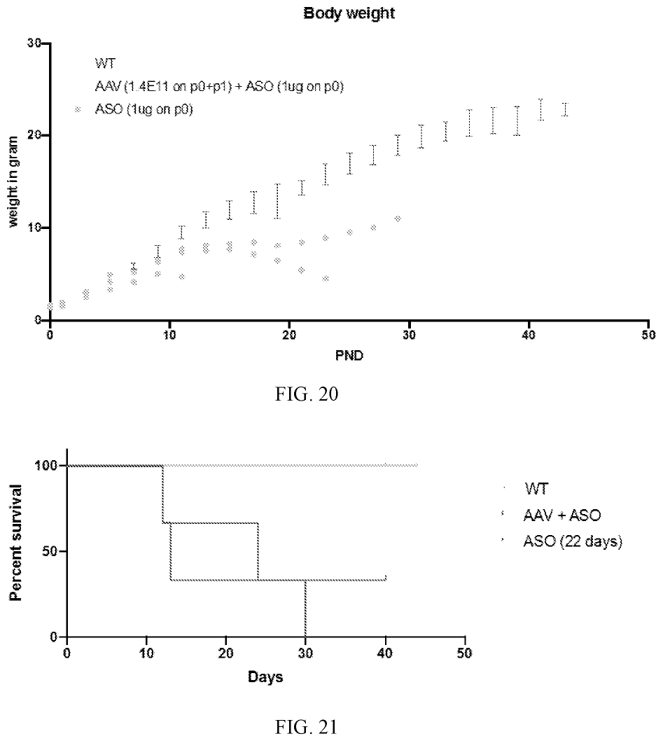

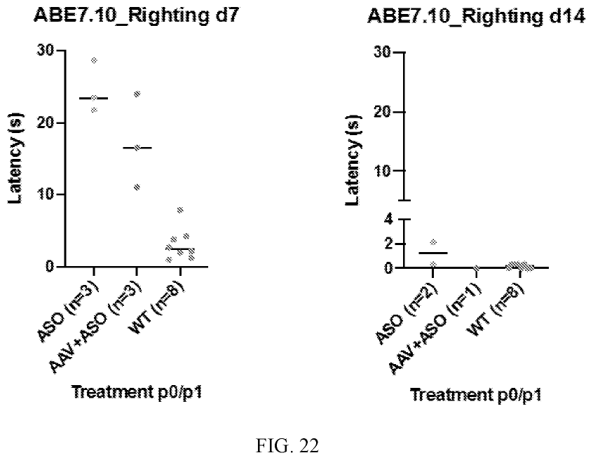

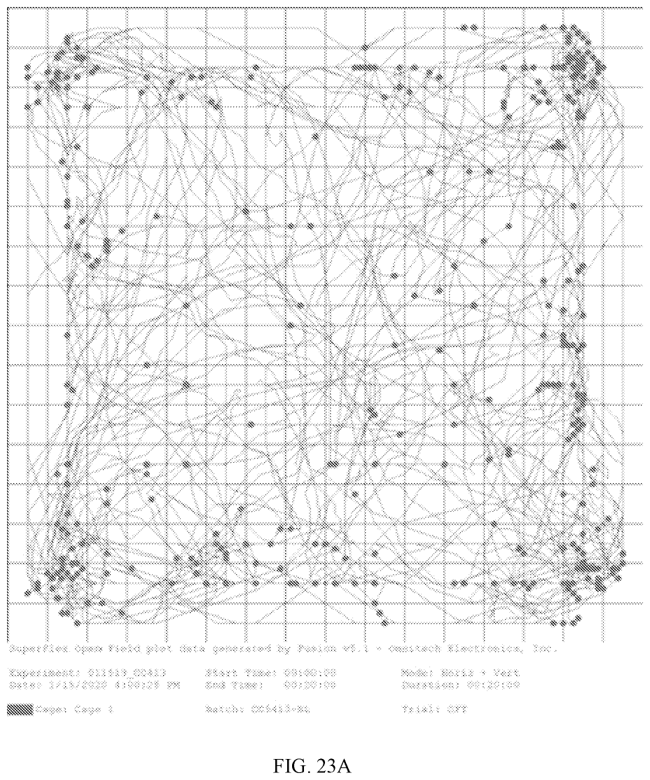

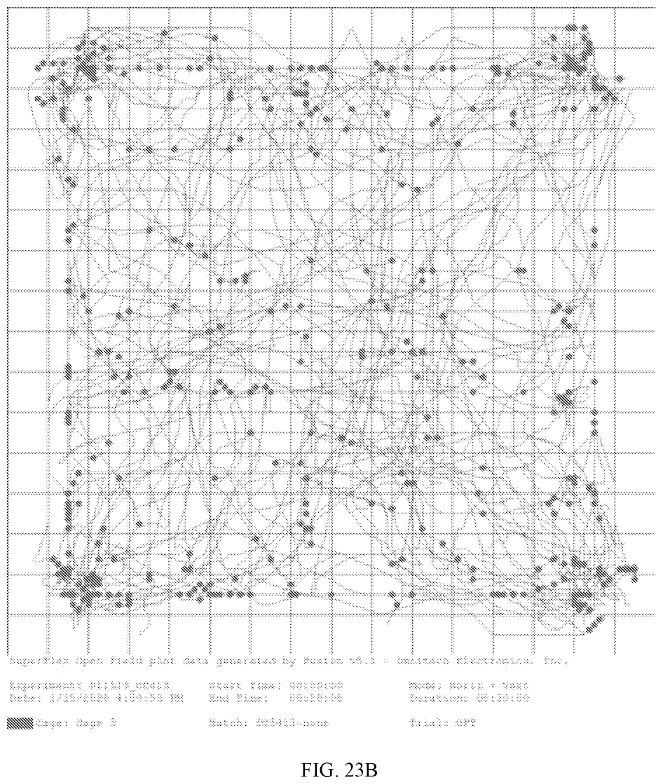

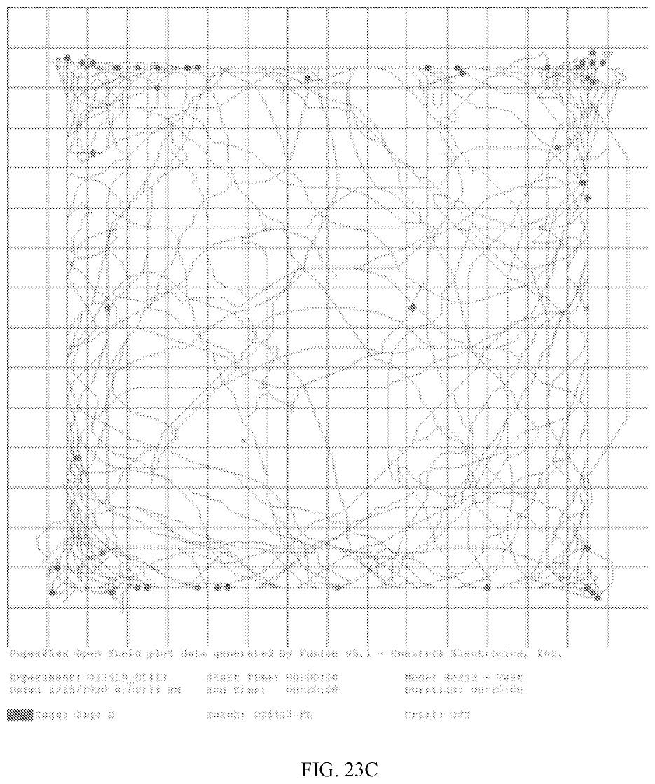

These disclosed CBEs exhibit low off-target editing frequencies, and in particular low Cas9-independent off-target editing frequencies, while exhibiting high on-target editing efficiencies. For example, the YE1-BE4, YE1-CP1028, YE1-SpCas9-NG, R33A-BE4, and R33A+K34A-BE4-CP1028 base editors may exhibit off-target editing frequencies of less than 0.75% (e.g., about 0.4% or less) while maintaining on-target editing efficiencies of about 60% or more, in target sequences in mammalian cells. (See, e.g., FIGS. 11, 15A, 15B and 17.) The Examples of the present disclosure suggest that CBEs with cytosine deaminases that have a low intrinsic catalytic efficiency (k_{cat}/Kₘ) for cytosine-containing ssDNA substrates exhibit reduced Cas9-independent off-target deamination.

In some embodiments, the fusion protein comprises an amino acid sequence that is at least 60%, at least 65%, at least 70%, at least 75%, at least 80%, at least 85%, at least 90%, at least 95%, at least 96%, at least 97%, at least 98%, at least 99%, or at least 99.5% identical to any one of the amino acid sequences set forth in any one of SEQ ID NOs: 223-248, or to any of the fusion proteins provided herein. In some embodiments, the fusion protein comprises an amino acid sequence that has 1, 2, 3, 4, 5, 6, 7, 8, 9, 10, 11, 12, 13, 14, 15, 16, 17, 18, 19, 20, 21, 22, 21, 24, 25, 26, 27, 28, 29, 30, 31, 32, 33, 34, 35, 36, 37, 38, 39, 40, 41, 42, 43, 44, 45, 46, 47, 48, 49, 50, or more mutations compared to any one of the amino acid sequences set forth in SEQ ID NOs: 223-248, or any of the fusion proteins provided herein. In some embodiments, the fusion protein comprises an amino acid sequence that has at least 5, at least 10, at least 15, at least 20, at least 25, at least 30, at least 35, at least 40, at least 45, at least 50, at least 60, at least 70, at least 80, at least 90, at least 100, at least 110, at least 120, at least 130, at least 140, at least 150, at least 160, at least 170, at least 200, at least 300, at least 400, at least 500, at least 600, at least 700, at least 800, at least 900, at least 1000, at least 1100, at least 1200, at least 1300, at least 1400, at least 1500, at least 1600, at least 1700, at least 1750, or at least 1800 identical contiguous amino acid residues as compared to any one of the amino acid sequences set forth in SEQ ID NOs: 223-248, or any of the fusion proteins provided herein. In some embodiments, the fusion protein (base editor) comprises the amino acid sequence of SEQ ID NO: 223, or a variant thereof that is at least 60%, at least 65%, at least 70%, at least 75%, at least 80%, at least 85%, at least 90%, at least 95%, at least 96%, at least 97%, at least 98%, at least 99%, or at least 99.5% identical.

In some embodiments, the base editor fusion proteins provided herein are capable of modifying a specific nucleotide base without generating a significant proportion of indels. An “indel”, as used herein, refers to the insertion or deletion of a nucleotide base within a nucleic acid. Such insertions or deletions can lead to frame shift mutations within a coding region of a gene. In some embodiments, it is desirable to generate base editors that efficiently modify (e.g. mutate or deaminate) a specific nucleotide within a nucleic acid, without generating a large number of insertions or deletions (i.e., indels) in the nucleic acid. In certain embodiments, any of the base editors provided herein are capable of generating a greater proportion of intended modifications (e.g., point mutations or deaminations) versus indels. In some embodiments, the base editors provided herein are capable of generating a ratio of intended point mutations to indels that is greater than 1:1. In some embodiments, the base editors provided herein are capable of generating a ratio of intended point mutations to indels that is at least 1.5:1, at least 2:1, at least 2.5:1, at least 3:1, at least 3.5:1, at least 4:1, at least 4.5:1, at least 5:1, at least 5.5:1, at least 6:1, at least 6.5:1, at least 7:1, at least 7.5:1, at least 8:1, at least 10:1, at least 12:1, at least 15:1, at least 20:1, at least 25:1, at least 30:1, at least 40:1, at least 50:1, at least 100:1, at least 200:1, at least 300:1, at least 400:1, at least 500:1, at least 600:1, at least 700:1, at least 800:1, at least 900:1, or at least 1000:1, or more. The number of intended mutations and indels may be determined using any suitable method. In some embodiments, to calculate indel frequencies, sequencing reads are scanned for exact matches to two 10-bp sequences that flank both sides of a window in which indels might occur. If no exact matches are located, the read is excluded from analysis. If the length of this indel window exactly matches the reference sequence the read is classified as not containing an indel. If the indel window is two or more bases longer or shorter than the reference sequence, then the sequencing read is classified as an insertion or deletion, respectively.

In some embodiments, the base editors provided herein are capable of limiting formation of indels in a region of a nucleic acid. In some embodiments, the region is at a nucleotide targeted by a base editor or a region within 2, 3, 4, 5, 6, 7, 8, 9, or 10 nucleotides of a nucleotide targeted by a base editor. In some embodiments, any of the base editors provided herein are capable of limiting the formation of indels at a region of a nucleic acid to less than 1%, less than 1.5%, less than 2%, less than 2.5%, less than 3%, less than 3.5%, less than 4%, less than 4.5%, less than 5%, less than 6%, less than 7%, less than 8%, less than 9%, less than 10%, less than 12%, less than 15%, or less than 20%. The number of indels formed at a nucleic acid region may depend on the amount of time a nucleic acid (e.g., a nucleic acid within the genome of a cell) is exposed to a base editor. In some embodiments, an number or proportion of indels is determined after at least 1 hour, at least 2 hours, at least 6 hours, at least 12 hours, at least 24 hours, at least 36 hours, at least 48 hours, at least 3 days, at least 4 days, at least 5 days, at least 7 days, at least 10 days, or at least 14 days of exposing a nucleic acid (e.g., a nucleic acid within the genome of a cell) to a base editor.

Some aspects of the disclosure are based on the recognition that any of the base editors provided herein are capable of efficiently generating an intended mutation, such as a point mutation, in a nucleic acid (e.g. a nucleic acid within a genome of a subject) without generating a significant number of unintended mutations, such as unintended point mutations. In some embodiments, an intended mutation is a mutation that is generated by a specific base editor bound to a gRNA, specifically designed to generate the intended mutation. In some embodiments, the intended mutation is a mutation associated with a disease or disorder. In some embodiments, the intended mutation is an adenine (A) to guanine (G) point mutation associated with a disease or disorder. In some embodiments, the intended mutation is a thymine (T) to cytosine (C) point mutation associated with a disease or disorder. In some embodiments, the intended mutation is an adenine (A) to guanine (G) point mutation within the coding region of a gene. In some embodiments, the intended mutation is a thymine (T) to cytosine (C) point mutation within the coding region of a gene. In some embodiments, the intended mutation is a point mutation that generates a stop codon, for example, a premature stop codon within the coding region of a gene. In some embodiments, the intended mutation is a mutation that eliminates a stop codon. In some embodiments, the intended mutation is a mutation that alters the splicing of a gene. In some embodiments, the intended mutation is a mutation that alters the regulatory sequence of a gene (e.g., a gene promotor or gene repressor). In some embodiments, any of the base editors provided herein are capable of generating a ratio of intended mutations to unintended mutations (e.g., intended point mutations:unintended point mutations) that is greater than 1:1. In some embodiments, any of the base editors provided herein are capable of generating a ratio of intended mutations to unintended mutations (e.g., intended point mutations:unintended point mutations) that is at least 1.5:1, at least 2:1, at least 2.5:1, at least 3:1, at least 3.5:1, at least 4:1, at least 4.5:1, at least 5:1, at least 5.5:1, at least 6:1, at least 6.5:1, at least 7:1, at least 7.5:1, at least 8:1, at least 10:1, at least 12:1, at least 15:1, at least 20:1, at least 25:1, at least 30:1, at least 40:1, at least 50:1, at least 100:1, at least 150:1, at least 200:1, at least 250:1, at least 500:1, or at least 1000:1, or more.

VII. gRNAs

Some aspects of the invention relate to guide sequences (“guide RNA” or “gRNA”) that are capable of guiding a napDNAbp or a base editor comprising a napDNAbp to a target site in a gene or target sequence (e.g., a C840T point mutation in SMN2). In various embodiments base editors (e.g., base editors provided herein) can be complexed, bound, or otherwise associated with (e.g., via any type of covalent or non-covalent bond) one or more guide sequences, i.e., the sequence which becomes associated or bound to the base editor and directs its localization to a specific target sequence having complementarity to the guide sequence or a portion thereof. The particular design aspects of a guide sequence will depend upon the nucleotide sequence of a genomic target site of interest (e.g., the mutant T840 residue of human SMN2) and the type of napDNA/RNAbp (e.g., type of Cas protein) present in the base editor, among other factors, such as PAM sequence locations, percent G/C content in the target sequence, the degree of microhomology regions, secondary structures, etc.

In certain embodiments, the present disclosure relates to guide RNA sequences that may be selected and/or predicted for use in base editing by a user of the BE-Hive algorithm. In addition, the disclosure provides guide RNAs that may be used to train the BE-Hive algorithm. Examples of specific guide RNAs that are predicted to effectively introduce edits to a target sequence of interest based on Example 1 are as follows:

### Optimized Guide RNA Spacers Identified by BE-Hive Algorithm in Example 1

The following table comprises a listing of 2,749 sgRNAs (i.e., protospacers associated therewith) selected by BE-Hive of Example 1 using at least one base editor and wherein said one at least one base editor demonstrated at least 50% correction precision to the wild-type genotype among edited reads, or at least 70% correction precision to the wild-type genotype among edited amino acid sequences.

TABLE 5

| | PROTOSPACER ASSOCIATED | SEQ |
|---|---|---|
| INDEX | WITH EACH OF 2,749 sgRNA | ID |
| NO. | SELECTED BY BE-HIVE | NO: |
| 1 | TCACGAAAAAGCCAAGATGC | 451 |
| 2 | CTGTACAGGCCACATTGAGA | 452 |
| 3 | AGAGATCCGGACAGAATCGC | 453 |
| 4 | CAGCCACATGGTGTCGCGGC | 454 |
| 5 | GGTTTTACAGAGATCCCTTC | 455 |
| 6 | CAGCCGGTGCAGGGTGCCCA | 456 |
| 7 | CACTGAGTGGTACAAGAACG | 457 |
| 8 | TGCCTATCGCCACAGCAAGC | 458 |
| 9 | AACACTGAGTGGTACAAGAA | 459 |
| 10 | TGGCTGCTCGGTCTCCAGGG | 460 |
| 11 | TTCCCAGTGGGCACAGAGGA | 461 |
| 12 | CACCGTGGTTTACACCGACA | 3224 |
| 13 | CTTCCATGGGGGCCATGAGG | 3225 |
| 14 | GTGTGACACTGATATCCGCA | 3226 |
| 15 | CTACCCGTTAAAGAATCATC | 3227 |
| 16 | TGTAGCAGGTGAAGATGATC | 3228 |
| 17 | CTTGCGTTATGATGGATTCA | 3229 |
| 18 | GAATTTGCCGGTGACCGGGG | 3230 |
| 19 | ATGTGACGGAAGAGGTTGAA | 3231 |
| 20 | GGAGCAGGGGCTCAGCAGGG | 3232 |
| 21 | CAAGGATCCGGAGGAGGTGA | 3233 |
| 22 | ATGTCGGAGGCTTGGAACTC | 3234 |
| 23 | GAGGTTCCTTGAGTCCTTTG | 3235 |
| 24 | TGAACGCAGATTCTTGTTCT | 3236 |
| 25 | CCTTCCAGAGATTCTGGGGC | 475 |
| 26 | GTTCACTGATAGCAGGAAGG | 3237 |
| 27 | CCAGCCGGTGCAGGGTGCCC | 477 |
| 28 | CAGCGGTACAGGGTGACCAC | 478 |
| 29 | TGTCCAGGCAGGAGGCCAGG | 479 |
| 30 | TGCCCTTGTGCACAATGCCC | 480 |
| 31 | CCAAATTTGGATGTTTTCAA | 481 |
| 32 | CTCGGACAAAGTTCGGGCTC | 482 |
| 33 | CAATGCAGAGTGAGGTTGGT | 483 |
| 34 | ACGGGATTTTTTCATTTCTG | 484 |
| 35 | AAAATTCGCAAGTATGTCTT | 485 |
| 36 | CATTGATGCTGGCGCCCGGC | 486 |
| 37 | ATGTAATACACACTGATTGC | 487 |
| 38 | CTGAAGCTGGTCTGACCTCA | 488 |
| 39 | ACACTAATTCCTATGAATGT | 489 |
| 40 | GAAATACGATGGGGCGCTCA | 490 |
| 41 | GAAGCAGAGGCGGTAGGCGT | 491 |
| 42 | GCTTCCGAGGCTGCACCGCA | 492 |
| 43 | TGGAATCCTTTTATATTTAG | 493 |
| 44 | GTCCGCAACGGGCTAATCCA | 494 |
| 45 | GGGTTTTACGACCAGCAGCG | 495 |
| 46 | TCCCACGGAATCCTCCAGAT | 496 |
| 47 | CAGCTACCGGACGCTGGACC | 497 |
| 48 | TCCGGGAGATGAAATGGGAT | 498 |
| 49 | TCCCGCTGGACGGCTCCGAG | 499 |
| 50 | GGACAATCCGGTGGAGCGCC | 500 |
| 51 | AGGAGCGCTATTTAGCATCG | 501 |
| 52 | CACCATTGGCCGCACACTGG | 502 |
| 53 | AATAGGAGCAGGGGCTCAGC | 503 |
| 54 | CTTGTTACAGTAATAGCTGT | 504 |
| 55 | AGCGTGGGGTATGCCGTTGT | 505 |
| 56 | AAAGTACCGGATGACCCTCT | 506 |
| 57 | GCCGAACGCATCAACACTGC | 507 |
| 58 | CTCCATGGTGTGGGCGCAGG | 508 |
| 59 | CCCTGTCTTTGGCAGCGAAA | 509 |
| 60 | ATTTTGGTACCTTTGTCCCC | 510 |
| 61 | CAGCAGAGCTTGCGGCGCCG | 511 |
| 62 | ATCCATTCCTGAATGGAACA | 512 |
| 63 | GAGGTACTCGGCAGATCCTT | 513 |
| 64 | TGTACAGGCCACATTGAGAT | 514 |
| 65 | AGTCAGTACCTGCCTGCCCA | 515 |
| 66 | ATAGACAGTGGCTGCACTTC | 516 |
| 67 | GACAGGCCCATGAAAACCAG | 517 |
| 68 | TCCGGAGGAGGTGAAGGCCA | 518 |
| 69 | ATAGCGGATCAGGGAGAAGT | 519 |
| 70 | TACCCGGATATCAAGAACTT | 520 |
| 71 | TAGAGCTCTGCTTGAAACAT | 521 |
| 72 | CGGAGGGTGAGGAGTGCAGG | 522 |
| 73 | GGCGATGTCATCACCTTTGA | 523 |
| 74 | AGTGGCCGTACAGGGAGATT | 524 |
| 75 | GTCGAGACGCTGGCCAGCTA | 525 |
| 76 | GTGACGGTGAGCTCTGCAAA | 526 |
| 77 | TTTCTACCGCAGCAGGCTAC | 527 |
| 78 | GCAGGTACCTGGGGGGCGCC | 528 |
| 79 | GTGGAGACGGACGCTGCACC | 529 |
| 80 | GTAGTACCGTGGGTACTCGA | 530 |
| 81 | TGTTTCTAACGGGACCACTG | 531 |
| 82 | GGAATTCCGGGAGATGAAAT | 532 |
| 83 | CCACCGTGGTTTACACCGAC | 533 |
| 84 | ACGGCACTGGGGGTCTTGTT | 534 |
| 85 | GTTCCCAGTGGGCACAGAGG | 535 |
| 86 | AAGGCGTGGGGAAGCATTAA | 536 |
| 87 | TGAGCCGGTGGGCCCAATGC | 537 |
| 88 | AGGTCGTGTAAAATGGATAA | 538 |
| 89 | TATGACCGGCGGCTGGAGCC | 539 |
| 90 | CTACAATTCCTACCTGTCCG | 540 |
| 91 | GTTGAATCTGTGTGTAAACG | 541 |
| 92 | ACACTGAGTGGTACAAGAAC | 542 |
| 93 | ACTAACCTGGGGCTGCCCTT | 543 |
| 94 | GATTACCATCCCTCCATTCC | 544 |
| 95 | TTCACTGTGGTCATTTTCCT | 545 |
| 96 | GCATGTCCTACATCCCGCAG | 546 |
| 97 | TACAGACGTGAATGCTTCCC | 547 |
| 98 | GAAACCGTAGAGGCAGCTGC | 548 |
| 99 | GAAATAGTACCTTTCTTGAA | 549 |
| 100 | AGACGATGGGGGGCCGCAGC | 550 |
| 101 | GGTGTTGCTGGAATGGAGAA | 551 |
| 102 | CCTGAAAAGCCCAAACTCCC | 552 |
| 103 | GTACGTGCCTGTGAACGGCA | 553 |
| 104 | AACCTCATGGGATTCAACTG | 554 |
| 105 | AACGTGAAAGAGATGCCTGT | 555 |
| 106 | GTGCTGCCGGGCATATTTTC | 556 |
| 107 | CGACAGGCCCATGAAAACCA | 557 |
| 108 | ACCGGACATCCTCAGTGCCG | 558 |
| 109 | TAACTGTGCCCATGGCCATT | 559 |
| 110 | CAGGGCTGAGGGGAGAGGAC | 560 |
| 111 | GACGCGGGCGACCGGGTAAG | 561 |
| 112 | GATCATGCCGTCGTACAAGG | 562 |
| 113 | GAACGCTGTCCACCGTGGAG | 563 |
| 114 | GAGTTTCGCTCTTGTCGCCC | 564 |
| 115 | GCTGTCCTCTCCAGCTCCAG | 565 |
| 116 | TCTGCGGGCAGCTGGTCTTC | 566 |
| 117 | GACAGAAAGTGGTAGCAAAG | 567 |
| 118 | ACGTTTCTGCTGATCGTGCT | 568 |
| 119 | CTGTGTGCGCCAGGGCTGTG | 569 |
| 120 | TGGAAGATCTATGAGGAATG | 570 |
| 121 | TCAAGCGGTTCAAGGGCAAA | 571 |
| 122 | ATCACTGCTGACGGTGGAGT | 572 |
| 123 | TATCTGTGCGAGGGTGCTCG | 573 |
| 124 | AACCGAAGGCTGGTGGCCAC | 574 |
| 125 | GGCGGGTAGAGGGTCTGCAG | 575 |
| 126 | TGCCGGAGGGTGAGGAGTGC | 576 |
| 127 | AAGGACTCCCCTTGCAATAA | 577 |
| 128 | AAAACGTGTTGGTGCTTGAG | 578 |
| 129 | TTTGGTTGGCCCTGTTGGCT | 579 |
| 130 | GACGAGGATCTCTAGGGTGG | 580 |
| 131 | TCCGAGTCAGATCTGCAATC | 581 |
| 132 | CACAGTGGGCAAGACCTCTC | 582 |
| 133 | AAGTAGCATAAATTTGTGCA | 583 |
| 134 | AAACAGAAAGCGGACAATCA | 584 |
| 135 | TCACCGAGAAGGTGCCTCTT | 585 |
| 136 | ATCGCGGACTACAACATCAT | 586 |
| 137 | AAGTATGTGCGGAGCGCCTC | 587 |
| 138 | TGGGGCTTCCTCTCGGGCCG | 588 |
| 139 | ACGGTAGTAAGTAGCCACAT | 589 |
| 140 | CTGTGAAGACTTCGAACAGC | 590 |
| 141 | GAGGTGCGCAGGCGCGTGTG | 591 |
| 142 | GTAGAAGCAGATTTTCTGCC | 592 |
| 143 | GGCTAAGGGCCACGGCAAGA | 593 |
| 144 | GCTGAACTGCAGGGGGCATG | 594 |
| 145 | AGAGCAAGCGTAGACAGCCG | 595 |
| 146 | TACGTGCCTGTGAACGGCAA | 596 |
| 147 | GTGCGGAAGAAAAACTCAGT | 597 |
| 148 | GAACGTGAAAGAGATGCCTG | 598 |
| 149 | CCAATGTCACTGTGGTGGAC | 599 |
| 150 | GAAAACGTGTTGGTGCTTGA | 600 |
| 151 | CTGCGTGACTCCGACTGGAC | 601 |
| 152 | CGGACTACAACATCATTGGC | 602 |
| 153 | ACAGATGGAAGCTATCTGAA | 603 |
| 154 | TGGATACGTCCCAGTATTTT | 604 |
| 155 | TGCAGTAGGTACGCGGCGGC | 605 |
| 156 | CCCGGATATCAAGAACTTTG | 606 |
| 157 | ACCGTACAAAAGGACAGCAG | 607 |
| 158 | CATGCTTCTGCTGATCGTGC | 608 |
| 159 | AGAAGCAGAGGCGGTAGGCG | 609 |
| 160 | TCTGGCCGGACCGAGGAACC | 610 |
| 161 | GGAGGCAAACGGGTTCCTTG | 611 |
| 162 | CGGCCGGAAGTTCGAGAAGC | 612 |
| 163 | TTCCGGGCCGGGACCGTGAT | 613 |
| 164 | TACACTGGGTGATCCTGCAA | 614 |
| 165 | GGTTTTACGACCAGCAGCGA | 615 |
| 166 | TGTTGAGGACGGAAGAGCAG | 616 |
| 167 | CACGTGCAACCTGGCCTTTG | 617 |
| 168 | TTCTTCGCGGACTGCAAACA | 618 |
| 169 | GACGGAAGAAGGATGGGCAG | 619 |
| 170 | TCCGGGTGTCATCAGCTTGT | 620 |
| 171 | CTCAACGGTACTTGTGAGCC | 621 |
| 172 | CCGTTCTTCAGGCCCATCAT | 622 |
| 173 | AAGCAAGTTTTGGTTTCATT | 623 |
| 174 | TGTGGTGGACCGGCTGTCAC | 624 |
| 175 | GGCGCTGCTGCTGAAGATGC | 625 |
| 176 | CCGTATAGGCCACATTGAGA | 626 |
| 177 | ACGGGCAAAGAAGGTGTCCA | 627 |
| 178 | ACAGTGCCTGCACCCAGCGC | 628 |
| 179 | CTGGTGGCAACAGACCCGTC | 629 |
| 180 | GAGCGTGGGGTATGCCGTTG | 630 |
| 181 | AAAGAACCTGTAGATCAAAG | 631 |
| 182 | AGGTAGATTCCAATGGCTTC | 632 |
| 183 | GTAAGTGCTGACCAAATTAC | 633 |
| 184 | AAGCATGTGTGGAGCGCCTC | 634 |
| 185 | AGGTCACTAGACATGAATAA | 635 |
| 186 | CGATGGTTGGCGGTTTAGAC | 636 |
| 187 | AGCTGAATTTGTGTGTAAAC | 637 |
| 188 | ACACAGTTCTCAAACACTGT | 638 |
| 189 | CACTGATGTGCTCCAGGGTC | 639 |
| 190 | CATCCATTCCTGAATGGAAC | 640 |
| 191 | CATCATGCCCATCCTGGAAG | 641 |
| 192 | TCATTGTCGTAGGTAAAGAA | 642 |
| 193 | AAGTCACAGTGCACGGCACA | 643 |
| 194 | GAAGGAGACGGCCTCCATGA | 644 |
| 195 | GCAAGGTGAGGTGGTGACAA | 645 |
| 196 | TTTGGCACGAATGAAAAGGT | 646 |
| 197 | AGAGGGAAACCTTTCATCAG | 647 |
| 198 | GTGTAGCGTATGCTTCCAGG | 648 |
| 199 | AAACTGGCCCTTATACCTGT | 649 |
| 200 | GATCACTGGTAACTCAGTAG | 650 |
| 201 | ATATGCGCATCTGTGGACCC | 651 |
| 202 | GGAAATACTGAAAGCAAAGA | 652 |
| 203 | TGTGTGCCGGCCCATCACTT | 653 |
| 204 | CGGCACTGGGGGTCTTGTTC | 654 |
| 205 | CCAGGCGGTCCGCAAGGCCC | 655 |
| 206 | CTGTGGTGGACTGGCTGTCA | 656 |
| 207 | CACCATCGATGTGGCCCCCT | 657 |
| 208 | GTACCTGCACTGGGCTGACT | 658 |
| 209 | CAGGCAACTGGTTTAAGAAC | 659 |
| 210 | TAGTACCGTGGGTACTCGAA | 660 |
| 211 | GGGGCCGTGACGACCAGCCC | 661 |
| 212 | GAGGTGAGCAATCTGTCAGC | 662 |
| 213 | TGTTGTTACTGGAATTAGTT | 663 |
| 214 | GCTGGTACTCGTAATCCGGG | 664 |
| 215 | ACAGAAAGTGGTAGCAAAGC | 665 |
| 216 | CTTCCGGGCCGGGACCGTGA | 666 |
| 217 | GATGGTTCCGCTCCAGGACC | 667 |
| 218 | AGAAGGAACGCTGTCCACCG | 668 |
| 219 | ACGTGAATGCTTCCCTGGAC | 669 |
| 220 | TTTGTCTGACAACAATACAT | 670 |
| 221 | GCTGGAATGGAGAATGGCCT | 671 |
| 222 | AGGAGAACTTCTGGATTTGC | 672 |
| 223 | GTGCCTCATCAAGCTACCCA | 673 |
| 224 | ATCCCGGACAGCTTCCCCAA | 674 |
| 225 | ATGCTTCTGCTGATCGTGCT | 675 |
| 226 | CACCTGTGACGGCTCTGAGG | 676 |
| 227 | TTACGCAATGGAACGCCCGA | 677 |
| 228 | AGTACCGTGGGTACTCGAAG | 678 |
| 229 | CCTCGTAGTAAATCCAGTTC | 679 |
| 230 | CCATGTGGTGGAAGAGATAT | 680 |
| 231 | CAAGGTCCGTGTCTTTTCCT | 681 |
| 232 | TCAACGGTACTTGTGAGCCA | 682 |
| 233 | AACCGGTTCTACACGCGAGC | 683 |
| 234 | TGGAATAGCGTTTGCCACAG | 684 |
| 235 | TGACGGAGGGGATGGCGCCT | 685 |
| 236 | CAGGACCAGCATCATCCCCC | 686 |
| 237 | AGCGGGCAAGGTGGCAGAGA | 687 |
| 238 | TGTCACTGAAGACCCCGAGC | 688 |
| 239 | TTCCGGGGGGCCTCAGGGCG | 689 |
| 240 | GCAGGTGAACCGTTTCCCCT | 690 |
| 241 | TATGAACGTTGGTGTCCCTT | 691 |
| 242 | AGTTGTCCCAATACCTGCTT | 692 |
| 243 | CTGCTTCGCGGGCACGGCTG | 693 |
| 244 | TCTGGTGGGCACGCAGCAGC | 694 |
| 245 | CATTCCGTTCTCAGTTTTCC | 695 |
| 246 | GGTACCTGCACTGGGCTGAC | 696 |
| 247 | CAAGCGGTTCAAGGGCAAAT | 697 |
| 248 | TGATCTCTTGGGAGAAGAAC | 698 |
| 249 | GGTGACCTACCCGGACTCAG | 699 |
| 250 | ATTCCAATGGCTTCTGGGTC | 700 |
| 251 | ACGATGAGCGCAGCGAAATT | 701 |
| 252 | TGAGGCTGGTGTCAATCCTT | 702 |
| 253 | GTTTTACAGAGATCCCTTCC | 703 |
| 254 | TGGGCCGGGGCCTTCTGGGC | 704 |
| 255 | AAGCAGATTTTCTGCCAGGT | 705 |
| 256 | AGCTGTGCGATGAAGCAGGC | 706 |
| 257 | GATCACTGGCAATTCAGTGG | 707 |
| 258 | GAGGACGAGGATCTCTAGGG | 708 |
| 259 | CACCGTGGAACTGGCCCAAC | 709 |
| 260 | CTTCACGGTGTGGGCGCAGG | 710 |
| 261 | CACGTTTCTGCTGATCGTGC | 711 |
| 262 | GGCAGGTCTCATTGAAGGTA | 712 |
| 263 | ACGACCCGCTGGACCTCACT | 713 |
| 264 | ACCTGCGGGTGCGTGGCTGC | 714 |
| 265 | AATTCCGGAGTATCGGCCAT | 715 |
| 266 | TTCTCCGGCTTAGAGGTGAC | 716 |
| 267 | GGGTCGGAATGACCCAGATA | 717 |
| 268 | CAAGGGCTGTGGCCGGCAAC | 718 |
| 269 | TGTACACAGTTGCAACACCT | 719 |
| 270 | GGTCCCGGATGTGGTGAGGA | 720 |
| 271 | GGTGGGTTACGGTCTTCAAA | 721 |
| 272 | ATACTGCCGGGAAGAAGCAA | 722 |
| 273 | GCAGCAGAGCTTGCGGCGCC | 723 |
| 274 | CTCCTGGAGGGTGCTGTTCA | 724 |
| 275 | CTGTACACAGTTGCAACACC | 725 |
| 276 | AGGTGAGCAATCTGTCAGCA | 726 |
| 277 | CTGTCCTCTCCAGCTCCAGG | 727 |
| 278 | TCCATGGGGGCCATGAGGTG | 728 |
| 279 | CATAGCGGATCAGGGAGAAG | 729 |
| 280 | GTACAGAGGTATTGTTCTTT | 730 |
| 281 | AATACGGGAAAAAGGCGTGG | 731 |
| 282 | GAAGCATGTGTGGAGCGCCT | 732 |
| 283 | AAGAACCCGGGCACGCTCTT | 733 |
| 284 | GAGGCTGGTGTCAATCCTTC | 734 |
| 285 | CCAGCGGTACAGGGTGACCA | 735 |
| 286 | TTGGCACGAATGAAAAGGTT | 736 |
| 287 | CAGCCCGGGCGGCGGCGGCG | 737 |
| 288 | ACAGCGAAATCTCGATGGAG | 738 |
| 289 | GGTGGCACTGGAAGGGGAAG | 739 |
| 290 | CCGGGAGATGAAATGGGATT | 740 |
| 291 | GTCTGATGCACTGTGTGCAG | 741 |
| 292 | GACGTTGTAGTCCACGATGC | 742 |
| 293 | TTGCACCATTGGCCGCACAC | 743 |
| 294 | GCCCTGCCGTACCCGCTGCC | 744 |
| 295 | GCGGTTCAAGGGCAAATGGG | 745 |
| 296 | TTTACGCAATGGAACGCCCG | 746 |
| 297 | TGCACAACAGCACCCGCGAC | 747 |
| 298 | GGACCGGCTGTCACGGGCTC | 748 |
| 299 | GCACTGTGTACTCCTGTGAG | 749 |
| 300 | GAAGCAGATTTTCTGCCAGG | 750 |
| 301 | GGTGACGGTGAGCTCTGCAA | 751 |
| 302 | TGTGAGATCCGCCCTTTCCA | 752 |
| 303 | CCACAGCAAACCAGTAAATC | 753 |
| 304 | TACTGAGAGCACAGCGCAGC | 754 |
| 305 | AAGCTCCGAGGTCCTGGGGG | 755 |
| 306 | CGTGTGCTGGCCCATCACTT | 756 |
| 307 | TACCGCCGTGAATGCCCGCC | 757 |
| 308 | AACCTCCACTGGGCCGACAC | 758 |
| 309 | TTACCGTGCGAAGTTAACGT | 759 |
| 310 | TGTACTTTCTCCAGCTCCAC | 760 |
| 311 | CATTCGCGGTGGACGATGGA | 761 |
| 312 | GGAACACCGTCCATTGGCAT | 762 |
| 313 | CGGATGCTGCAGGTGCACAC | 763 |
| 314 | CCGCACTCCGACCTGAGCCA | 764 |
| 315 | GCAGACCGCAAGAATACCAT | 765 |
| 316 | TCATCTGGAACAGTCTACAA | 766 |
| 317 | CTGTCTCTTCCTCTAGAGTC | 767 |
| 318 | GGGCCGATCCAGCAGGTAAG | 768 |
| 319 | GCGGCTGGCCTTGGGATTGA | 769 |
| 320 | CCGTTACCCGGAGGGCCAAC | 770 |
| 321 | GCACCGCAGCCTGGCCAGCC | 771 |
| 322 | TTGGCCCCGTTGAGTCTATC | 772 |
| 323 | CGCCGGCCACCAGCACTGCC | 773 |
| 324 | ACTTTAAGTCCCTGTTTGTG | 774 |
| 325 | TCCGGAGGTAGGACCCGGCG | 775 |
| 326 | TTCTGACAGGCAGCCTGCAC | 776 |
| 327 | TGCTCTCGATTCGACTTAAA | 777 |
| 328 | GTTCACGACAACGTGCACAG | 778 |
| 329 | TCTCGGCAACTGAGCGAATT | 779 |
| 330 | CCGAAGGCTTCAATTTCCAC | 780 |
| 331 | TTGAACCTGCAACCGGTTCT | 781 |
| 332 | GCTGATCTTCAGCCTCCTTT | 782 |
| 333 | GCACCCGCGTCTCCTGGTCG | 783 |
| 334 | GGCCAACGCATTAATACAGT | 784 |
| 335 | TGTTCTGGTCCTGCTTTGAG | 785 |
| 336 | TCATTGCAAGGGAAGTCCTT | 786 |
| 337 | TGCAAACCGGGCCTTCTCAC | 787 |
| 338 | CCGGGTCGGGCCAGTGCCCA | 788 |
| 339 | CACCACGGAGAAGCATAAAG | 789 |
| 340 | CCGCGCGCCGCTTGCGCTCC | 790 |
| 341 | AGCCGCTACCGGTGTAATGA | 791 |
| 342 | TACGATGGGGCGCTCAGGGT | 792 |
| 343 | TGCAGACCGCAAGAATACCA | 793 |
| 344 | CTACCTCCCGGAGGCCGCAG | 794 |
| 345 | GGCCCGCCCGGACGGAAGGC | 795 |
| 346 | AGCGACAGGCCTGGAAAACC | 796 |
| 347 | TACATTTACAGGTCCCACGA | 797 |
| 348 | ACAAAGCTCCGAGGTCCTGG | 798 |
| 349 | AAAATACCTCACGGGAGAGG | 799 |
| 350 | GGGCTGGCAGCGCCAGGTGA | 800 |
| 351 | AAGATCACTGGCAATTCAGT | 801 |
| 352 | CTTCACCCGGGTCATGGCGC | 802 |
| 353 | TCTTTTCATATTTAGGGGTA | 803 |
| 354 | TTCCCCGATGAGGCAGATGC | 804 |
| 355 | CGCCGAGCGGAAACTTTTGT | 805 |
| 356 | ATTGCACGTCCCTGTTCACT | 806 |
| 357 | GTACTTTCTCCAGCTCCACT | 807 |
| 358 | ACAGCGCACCGGCATCGAGG | 808 |
| 359 | CATGACTCGGGCTGCAACCA | 809 |
| 360 | AGTCCACCTGGGGAGGAAGG | 810 |
| 361 | TGAATGGCAGCCAGGGTTGC | 811 |
| 362 | GGCCTGCAGTAGGTACGCGG | 812 |
| 363 | GGCAGACCACCAGCAGCCTA | 813 |
| 364 | AGACAGCGCACCGGCATCGA | 814 |
| 365 | TCTGTCCAGGATGCTCCCAA | 815 |
| 366 | CATGCGCCGCCTCGAGGCCT | 816 |
| 367 | CCAGGCTTCCCAAGGTTACC | 817 |
| 368 | GCGCCGCCTCGAGGCCTTGG | 818 |
| 369 | CACCTTCCCGTCGGTGTATG | 819 |
| 370 | TGGTGCGGTCCCGCGGGCAG | 820 |
| 371 | CCTGCGCGGGTGGTATCAGT | 821 |
| 372 | GCACACGTCCCAACAGCTCA | 822 |
| 373 | GTCGAACGCCCGGGTGGAGG | 823 |
| 374 | CCCTGCCCTCCATCACCCAC | 824 |
| 375 | GCAGCCCGGAGCATGGGCTG | 825 |
| 376 | GTCGCCGCCCGTGGCCCCTG | 826 |
| 377 | CCACTGACAGCAGCGATGAC | 827 |
| 378 | TGCACTCGCCGTGGGTGCAG | 828 |
| 379 | CAGTGGCCGTACAGGGAGAT | 829 |
| 380 | GCGAATATCTTCTGCAATGG | 830 |
| 381 | CGTCGCCCAGGAGCTGTGGG | 831 |
| 382 | GAACACCGTCCATTGGCATG | 832 |
| 383 | TCACGTTGCAGCCGAGGTTC | 833 |
| 384 | GCACGTCGCCCAGGAGCTGT | 834 |
| 385 | TCAGTCTGGCAAAGAAGAAG | 835 |
| 386 | ATCCACCCGGGCCACCAGCC | 836 |
| 387 | GACAGCGCACCGGCATCGAG | 837 |
| 388 | AGACTTCGCTTTCCTTGGTC | 838 |
| 389 | CACCCGCAAGTCCCTGCCCA | 839 |
| 390 | CACTACTGGGGTCTCGGTCA | 840 |
| 391 | CATTCGGAAGAATGAACAGA | 841 |
| 392 | CTCTGCCTTGGATCCTAACC | 842 |
| 393 | ATCATCTGGAACAGTCTACA | 843 |
| 394 | TTCACGACAACGTGCACAGA | 844 |
| 395 | GGGCTGCTGCGCAGCGGCCG | 845 |
| 396 | GGACAACGTAGAAATACTCC | 846 |
| 397 | GAAATCCAAAGTACCTGTAG | 847 |
| 398 | TTTACCGTGCGAAGTTAACG | 848 |
| 399 | AGCCCCCTGTGTGCTCAAGG | 849 |
| 400 | AGACAGCTTCTCCTGAGAAT | 850 |
| 401 | CCAGACGTCGCCCAGGCCGA | 851 |
| 402 | AGGAACACCGTCCATTGGCA | 852 |
| 403 | GGTGCCATAGAAAAGGAGGA | 853 |
| 404 | CTCGCCGTGGGTGCAGAGGC | 854 |
| 405 | AATTCACCGTAAAGCTGGAA | 855 |
| 406 | ATGTATCAATTACAGACACT | 856 |
| 407 | TGCACACATATGTGCCAATG | 857 |
| 408 | CTGTGAGATCCGCCCTTTCC | 858 |
| 409 | CACGGGGCCTTCTACAGTGA | 859 |
| 410 | AGCACATCGCCCAAGAGCTG | 860 |
| 411 | CCGGATGAAGTAGCACACGA | 861 |
| 412 | ATACGGAGGTAATGGCATGT | 862 |
| 413 | CTTTACCCAGAGCTTCGTCC | 863 |
| 414 | CAGAAGTTGCTCAAATCCTG | 864 |
| 415 | CGACGCAGATGGTGATGCCC | 865 |
| 416 | GAGGAGCCCAATATGATCCA | 866 |
| 417 | CAGTGAAAGCACGGGCCAGC | 867 |
| 418 | CGAGCGGCTGCCGAGCCGGG | 868 |
| 419 | TGCACTTGCAGCGCCGGTTC | 869 |
| 420 | ATGTTGTCAGGGGCAATGTG | 870 |
| 421 | GTGCATGCTGCACAACTTTG | 871 |
| 422 | TACTCTTGCAGTCTGCATGC | 872 |
| 423 | TCCATTCCTGAATGGAACAG | 873 |
| 424 | CCTGCACCGGTACACGGGCG | 874 |
| 425 | TTCCGATAGGCAGCCTGCAC | 875 |
| 426 | GGATCGGCTTCACTGTGCTG | 876 |
| 427 | TTCCGGAGATTTATGTTCTA | 877 |
| 428 | CGTATAGGCCACATTGAGAT | 878 |
| 429 | TAGGCACACAGCTGACAAAG | 879 |
| 430 | ACTGGTTAGGCGGATCTGGT | 880 |
| 431 | TGCTGATGTTGCTGGACCAG | 881 |
| 432 | GGTGCGCCGCTTGGACATAC | 882 |
| 433 | TGGAGCCGGTAGCTAAAGAA | 883 |
| 434 | GGAGGCTCACGATGAGTGCC | 884 |
| 435 | ACAGAAGGAATAGGGACGAG | 885 |
| 436 | ACGACAGGAGAGGTCATAAC | 886 |
| 437 | CATGAACGCCTCCATGGTGT | 887 |
| 438 | TTTCTCGATACGGGGAGCTG | 888 |
| 439 | ATCGATCGCACCCTAAAAGC | 889 |
| 440 | AGCAGCCACGATAGCCCAGA | 890 |
| 441 | TGCTGACGAAGGTAGCAGGG | 891 |
| 442 | ACTGGAATCCAGAAACCAGT | 892 |
| 443 | CACGAAAAAGCCAAGATGCG | 893 |
| 444 | CGAGGTTGTCCAGGTGAGCC | 894 |
| 445 | CGGCTGGAGAGCATCCACCT | 895 |
| 446 | GTCCCCGCAGGGCATTGGCA | 896 |
| 447 | ATTCACCGTAAAGCTGGAAA | 897 |
| 448 | CCCCGCTCTCCATCACCCAC | 898 |
| 449 | CCACACTGGTTAGGCGGATC | 899 |
| 450 | CCTGTGCGTCCCCCAGGGGC | 900 |
| 451 | TCCACCCGGGCCACCAGCCA | 901 |
| 452 | CAGCCGCCCCTGCTGGGAGT | 902 |
| 453 | TGGGTGCCCAATAAGACCGA | 903 |
| 454 | GCCGCTGGATCCCGAAGGTG | 904 |
| 455 | GATGCGATGAAGGAGATGGG | 905 |
| 456 | ACACCCGCAAGTCCCTGCCC | 906 |
| 457 | GGCCGATCCAGCAGGTAAGT | 907 |
| 458 | TGCCACTGTCACTGTAGTCT | 908 |
| 459 | CGCCACCTGCGCGACTTCTG | 909 |
| 460 | AAGCTCCCGGACTTTTTTCT | 910 |
| 461 | ACGGCGAGTCGCATTCGTGC | 911 |
| 462 | CCTTCGCCACGCGCCTGGAC | 912 |
| 463 | ACGGAAGAGAAACTCATGAT | 913 |
| 464 | TGTGCACGATGTTGGAGGCT | 914 |
| 465 | GTCCGAGCTACAGCAAGATC | 915 |
| 466 | AACCGGCTATTGTTACCCAG | 916 |
| 467 | GCAGGAGCCCAACGTGACGG | 917 |
| 468 | GTGCACTGAGGGCCTTGGGG | 918 |
| 469 | TCCCCGTGCTGCTGGAGTTG | 919 |
| 470 | CACCATCCCGAGTGCAGACC | 920 |
| 471 | GCACGGCACCGTCACCAACT | 921 |
| 472 | GACCGCGTCCAGTTTCAGAG | 922 |
| 473 | CCGCACTGCCATCATTGCCC | 923 |
| 474 | CCAACAATGCAGAGTGAGGT | 924 |
| 475 | GGCTGATCACCTCACGCTCC | 925 |
| 476 | CAGCCTTGCCCAGTTTTCCC | 926 |
| 477 | CGATCTGAGTCATCTTCTCC | 927 |
| 478 | AGACAGAGGCAGAAATCGTG | 928 |
| 479 | CGTGCGAGTTGGCAGCGGCG | 929 |
| 480 | CGTTCGGCTTGTGGTGTAAT | 930 |
| 481 | GGTGCTCCCAGGTGGGGCCC | 931 |
| 482 | TTCGGTAACCTACAGCTCAC | 932 |
| 483 | ACCGAAGGCTGGTGGCCACA | 933 |
| 484 | CCGAAGTCGAAACCAGCGCT | 934 |
| 485 | AGCACATCTCACGGCCGCGC | 935 |
| 486 | TTCCTACTCGGACAAAGTTC | 936 |
| 487 | CAGCACAACACTGCTGCTGT | 937 |
| 488 | GACGCTGGCCAGCTACGGGC | 938 |
| 489 | CATGCTGCAGAAGACTTTGA | 939 |
| 490 | CGAAACGTGTATCTCCTCCC | 940 |
| 491 | TTAGTGACACTTGTGGGCCA | 941 |
| 492 | TTCCTCGCCCGTCAGGAGGA | 942 |
| 493 | TCCCGCTGGCCCTCGCGGCC | 943 |
| 494 | GCCGAGGAGGCTCTCTTCTG | 944 |
| 495 | CTCCCGGCGCTACGGAAGTG | 945 |
| 496 | CTGCCGCCAAAACTGAAGGC | 946 |
| 497 | GCCAACGCATTAATACAGTG | 947 |
| 498 | AAGGCCAGAATAGAAGGAAT | 948 |
| 499 | CTGTCCAGGATGCTCCCAAG | 949 |
| 500 | CTGCCGCGCGTGGCCCGAGG | 950 |
| 501 | GTACACGGCGGGCACGTTGA | 951 |
| 502 | ACCCAAACGAATATCTTGTG | 952 |
| 503 | CCTCGTAGGTGCTGGTGTCC | 953 |
| 504 | GCTGCCGCGCGTGGCCCGAG | 954 |
| 505 | AGTACACATCTCCTAACTTC | 955 |
| 506 | AGTCCACACGCATATGTGTA | 956 |
| 507 | TGCTGACGGCTCTGGTCAGC | 957 |
| 508 | TGACATGGGCATTCTGGGAA | 958 |
| 509 | GTTTAGTTCGATTTATAAGA | 959 |
| 510 | GAGAAGGCCGTGTAGGTAAG | 960 |
| 511 | GAACCCACCGGGTGACGATG | 961 |
| 512 | GCCGTCGTCGACGACGAGCG | 962 |
| 513 | ACCGTGGAACTGGCCCAACT | 963 |
| 514 | CTTTAAGTCCCTGTTTGTGC | 964 |
| 515 | GCGTATGGTCTCTTTGTTTC | 965 |
| 516 | GAGTCCTTTGCCCTTTTGAG | 966 |
| 517 | GTACTGCCTGTCTGGGGACA | 967 |
| 518 | GTGGAGCCGGTAGCTAAAGA | 968 |
| 519 | CGCCTGCACCTCTTCATGCC | 969 |
| 520 | TGAGCCTTCCGTGTTTTCAC | 970 |
| 521 | CTCATTGTTCCCGCCTTTCC | 971 |
| 522 | CTGCCGTGGGTGCAGAGGCT | 972 |
| 523 | GTTGGCCTCGGGATTGAGGG | 973 |
| 524 | CCCAGCACAGTTGGCAAACA | 974 |
| 525 | CCATCGACTGTGTGCATTTT | 975 |
| 526 | GCGTGGCTGCTCGGTCTCCA | 976 |
| 527 | TAGCACATTTGCAACAAGCT | 977 |
| 528 | TTGGCGGCTGCTGGATCCAC | 978 |
| 529 | GCAGATGACTCGGGCAAAGG | 979 |
| 530 | CCGGGCCTCGTCGCCCACAT | 980 |
| 531 | GATGCTGCCCGTGTTGAGCT | 981 |
| 532 | CCCATACGCCTGCCTTCCAA | 982 |
| 533 | CGGCGTGGAAGAGAGCATCA | 983 |
| 534 | CACGCAAAGGAAGGGCTACT | 984 |
| 535 | GCCGGAGCTTGAGAGAGACG | 985 |
| 536 | CCGTGTGGTCTCGCGATCAG | 986 |
| 537 | TGCCGCGGTGGCGCTGTCAG | 987 |
| 538 | CGTTGTTTTGGGACACCACT | 988 |
| 539 | ATTCGCGGTGGACGATGGAA | 989 |
| 540 | CCGGAGATGGCAATCGAAGC | 990 |
| 541 | CGAATCCGCACTCATCATCC | 991 |
| 542 | AAACTGCCGTTTAGATTACC | 992 |
| 543 | GCGACGCGCTCGTACTCAGA | 993 |
| 544 | GGTGTCGAACGCCCGGGTGG | 994 |
| 545 | GACGCTGGCCACGGCCACGA | 995 |
| 546 | GCGCTCTCGGCAAAGAACGC | 996 |
| 547 | GTGCGGCATTTGTCCTGCTC | 997 |
| 548 | GCCCTCGTTGCTCTCTGAGT | 998 |
| 549 | GCAGCCGCCCCTGCTGGGAG | 999 |
| 550 | GGCCGCGCATGTGTTCAGAA | 1000 |
| 551 | AGGTCCAAGGCCCAGGCTGG | 1001 |
| 552 | TTATCCGCTGCTCAAGGGAC | 1002 |
| 553 | ATGTGTTCGCGCAGGGAGCT | 1003 |
| 554 | AGCATACCGGCGGATGGTCC | 1004 |
| 555 | TGTGTTCGCGCAGGGAGCTC | 1005 |
| 556 | CGCCTCCATGGTGTGGGCGC | 1006 |
| 557 | GCACACCTTGAGGTCACGGC | 1007 |
| 558 | CCGAATGTCCTGGATTTCCA | 1008 |
| 559 | ACTCTTTGGGATCGACTTCC | 1009 |
| 560 | GGAAGCGGCGTGCTGTGCTG | 1010 |
| 561 | CGTGCCGCTAGACACGGACG | 1011 |
| 562 | AATGCGACTGCTGACAAAGA | 1012 |
| 563 | CCTGCGGGTGCGTGGCTGCA | 1013 |
| 564 | TCTACCCGGACCCTGCATAC | 1014 |
| 565 | AGAGATTCTGGGGCAGGCGT | 1015 |
| 566 | GAAAACCAATTGATGAGGTA | 1016 |
| 567 | TGCCTCATCAAGCTACCCAA | 1017 |
| 568 | GTACCTCACAAAAACAGTAG | 1018 |
| 569 | GATGAACAGCCACTGGGGCC | 1019 |
| 570 | GTGAGCGGCCTCTTTATATG | 1020 |
| 571 | CCCTGCGCGGGTGGTATCAG | 1021 |
| 572 | GCTCATGCCTGAGCTGGCCC | 1022 |
| 573 | AAGGTACACATGGTAGGATA | 1023 |
| 574 | GGTCCAAGGCCCAGGCTGGA | 1024 |
| 575 | GTGGCCGTACAGGGAGATTG | 1025 |
| 576 | GGCCTGATGGAGCCACCCCA | 1026 |
| 577 | AGAGACCGCCAACATGTCAC | 1027 |
| 578 | GTGAGATCCGCCCTTTCCAG | 1028 |
| 579 | TCGACCGGCAGACAGGCCCT | 1029 |
| 580 | CAACGTGCCGGTCTGTGTGC | 1030 |
| 581 | CTGACAGGCGTTGTCGGAGA | 1031 |
| 582 | AAAACCTACGCCATGGGTGG | 1032 |
| 583 | GCCAACAGACTGATTTCCTG | 1033 |
| 584 | AACCCCGGCTGTTGTTACCA | 1034 |
| 585 | GGCTGTCCTCTCCAGCTCCA | 1035 |
| 586 | GCAGGACCCGGAAGCCATCC | 1036 |
| 587 | ATAACGAATGCCCCATCGAT | 1037 |
| 588 | AAATTCGCAAGTATGTCTTA | 1038 |
| 589 | TCGTGGTGCCGCTACTGGGC | 1039 |
| 590 | TGGCATCGTTGATGACATTC | 1040 |
| 591 | TTGATACAAGCCCAGGAAAT | 1041 |
| 592 | AGGCTGCCACGCGGGAGACC | 1042 |
| 593 | AGAGCCCGGCCAAGTGCTGC | 1043 |
| 594 | GAAGACCGCCGAGGTGGGCG | 1044 |
| 595 | GAGGGCCCGGAGCCCCTGAG | 1045 |
| 596 | CATACAAAGAGGCCACTCAC | 1046 |
| 597 | GAGGCACAGGGCATGGGTGA | 1047 |
| 598 | TCAGTCATGCTGTCACAACT | 1048 |
| 599 | GGCACCGTGCGAGTTGGCAG | 1049 |
| 600 | GCATACCGGCGGATGGTCCA | 1050 |
| 601 | AAGAGGATCCCGGATGCTGC | 1051 |
| 602 | CTTGGCTTCCTGGGTGAGAA | 1052 |
| 603 | CATGACGCAGGCGCTGCTGG | 1053 |
| 604 | CACACGGCTGAGACGTTCCA | 1054 |
| 605 | TAAGGACCCGCAGCACCGGC | 1055 |
| 606 | GCGGCTCTGGAACCAGACCT | 1056 |
| 607 | CCGATCCGCACCTGGTGCGT | 1057 |
| 608 | GAGCTGCCCGCCGAAGAAGC | 1058 |
| 609 | CCACACCTGTGACGGCTCTG | 1059 |
| 610 | GACCCTCATCGGCAACAGCA | 1060 |
| 611 | CCACTGCGCTGCCCGCGCAG | 1061 |
| 612 | CGCCCGTGGCCCGGCTGCTG | 1062 |
| 613 | GATGCTGGCGCCCGGCTGGC | 1063 |
| 614 | GGACCATGCCTGGACGCCCA | 1064 |
| 615 | TGTCAAGAGAGCGAATGTCA | 1065 |
| 616 | CTGGCCGGACCGAGGAACCA | 1066 |
| 617 | CGCTCTCGGCAAAGAACGCT | 1067 |
| 618 | TAGATTTCCATGGGGAAGTA | 1068 |
| 619 | TGCGCCGCCTCGAGGCCTTG | 1069 |
| 620 | GCACTCCGCAGCAGTGGGCC | 1070 |
| 621 | GGCACAGTGGAAGATCTATG | 1071 |
| 622 | CTGCCGGATCCCCCTGCTGA | 1072 |
| 623 | GTACACTGGGTGATCCTGCA | 1073 |
| 624 | ATTCGGAAGAATGAACAGAA | 1074 |
| 625 | GGGGTCTCGATAATAAAATT | 1075 |
| 626 | ACATCGACTTTAAATGACTT | 1076 |
| 627 | GCCACTTCCAGAACTGCCCG | 1077 |
| 628 | CCAACCTGACCCGCTTCTTC | 1078 |
| 629 | CCTGTACCGCACGCTGTACT | 1079 |
| 630 | CGTGCCGGCCTCGCGCATGG | 1080 |
| 631 | TCACTGAGCCAGGGAACTAT | 1081 |
| 632 | CGCGCCCGGAGGAGGAAATC | 1082 |
| 633 | GCCGCTCACGCAGCTTGCGC | 1083 |
| 634 | GCGCAGTTCCTCCCGCTCGC | 1084 |
| 635 | CCTCCTTCACCTGCTTGAGG | 1085 |
| 636 | TGAACCTGCAACCGGTTCTA | 1086 |
| 637 | GCGCCTGGCCAGCCCTCCAC | 1087 |
| 638 | CATGCCGCTAGAGGAGGAAA | 1088 |
| 639 | ACGCCCGTGGTATGTTATGC | 1089 |
| 640 | TATCCGCTGCTCAAGGGACT | 1090 |
| 641 | GCGCCGAGCGGGGTTCATGT | 1091 |
| 642 | TGGCCACGACCAAGCCCGAC | 1092 |
| 643 | GCTCTTCCCGACACTGCAGC | 1093 |
| 644 | TGAAGCAGCACACGATGGCC | 1094 |
| 645 | TGCAGCAGCCGCCCTTCTGC | 1095 |
| 646 | GTGCAGCCGGGCTCGCTGCT | 1096 |
| 647 | CTCACCTCCAACATCACTGC | 1097 |
| 648 | AATACGTGCTGAAAATGATA | 1098 |
| 649 | AGAAATACGATGGGGCGCTC | 1099 |
| 650 | GCGACAGGCCTGGAAAACCA | 1100 |
| 651 | GCTCTCGCGAGCCGCCTTGG | 1101 |
| 652 | CCCACCGCTCCTGTGACAGC | 1102 |
| 653 | GACACGAAGCACACACAATA | 1103 |
| 654 | CAGCCATGGCCAGGCCCCAG | 1104 |
| 655 | CTTCCTGCTGCGGTCCCCAA | 1105 |
| 656 | TTCCGGAGTATCGGCCATGG | 1106 |
| 657 | TCCATCGTGGCCCAGGAAGG | 1107 |
| 658 | CCATGACGCAGGCGCTGCTG | 1108 |
| 659 | ACTCACGCTGGCGGTCATGC | 1109 |
| 660 | CTGCACCGGTACACGGGCGA | 1110 |
| 661 | ACAGCGGCAGCTGCCAGTGA | 1111 |
| 662 | CCAAGTTTCAGGATGCTTCT | 1112 |
| 663 | GGGCCCTGGCCCACGTACGG | 1113 |
| 664 | GCAGCCCCTCGGTCTGCAAC | 1114 |
| 665 | AAACTCCGTCCCCATGGCCG | 1115 |
| 666 | AGGCGTCGACCAGCGAGTAC | 1116 |
| 667 | GGAGACGGCCTCCATGAAGG | 1117 |
| 668 | GCCCGCCTTGTGTCCAAGAA | 1118 |
| 669 | ACTCTGCTGTTCGGAACTAC | 1119 |
| 670 | CAGTGCCGCCTGCATTCGCG | 1120 |
| 671 | GGACCCGCCTGTGGATGCAG | 1121 |
| 672 | TAAGCCCTGCCAGCCAAGCT | 1122 |
| 673 | TGCACAGGGAATTCCAAGAA | 1123 |
| 674 | CCTGCCGGCCAACTCCAAAG | 1124 |
| 675 | AGAACATCACTGGGGGCTAC | 1125 |
| 676 | CAGGTGGGGCCCGGGCATCC | 1126 |
| 677 | GGTAGATTCCAATGGCTTCT | 1127 |
| 678 | GACCACTGCGCTGCCCGCGC | 1128 |
| 679 | CCTGGGCTGCTGCGCCAGCA | 1129 |
| 680 | TGCACCTGCCGTGGGTGCAG | 1130 |
| 681 | ACGTGGTTGTGGTCGTTCTG | 1131 |
| 682 | GTACCGGACAGACGTGAGCG | 1132 |
| 683 | CACTGGGGACCGCAGCAAGA | 1133 |
| 684 | CAGGGCGTCACGGTCGGTAT | 1134 |
| 685 | TCATCCGTTTGCCTGCTAAG | 1135 |
| 686 | CTTGGCCTCGAGCCTCAGCG | 1136 |
| 687 | CGCCGCCATTACCCCGGCCA | 1137 |
| 688 | CGAGTATTCGCGCTCCGGCG | 1138 |
| 689 | TGCCACGAGCCTTCACCTTA | 1139 |
| 690 | TGCTGCACTGCCAATTGCTG | 1140 |
| 691 | GCCCTCCGTGTGCTCAAGGG | 1141 |
| 692 | GGCCCACAGGTGCAGTTCCA | 1142 |
| 693 | GATAAGTCTACCATCCTGCG | 1143 |
| 694 | GAGACCACAAGAGGCAGAGC | 1144 |
| 695 | AACCAGAGTAATAGCGGGTC | 1145 |
| 696 | TTGCTCTCGATTCGACTTAA | 1146 |
| 697 | TGCAACCTTGGCCTCGAGGG | 1147 |
| 698 | GACATCGACTTTAAATGACT | 1148 |
| 699 | AATTCACGCATTCAGACTCG | 1149 |
| 700 | TGACAGGCAGCCTGCACTGG | 1150 |
| 701 | CACACACACCACGCCCTCTT | 1151 |
| 702 | GGCGCTTCCGGGGGGCCTCA | 1152 |
| 703 | AAGCGCTCCTTCTTCGATGT | 1153 |
| 704 | TGACCGTACTGAAAAACAAA | 1154 |
| 705 | GGATTTTATCCGCTGCTCAA | 1155 |
| 706 | GGACCTGCCCAACGTGAAGA | 1156 |
| 707 | GCGGCAACGGTGTAGCGGCG | 1157 |
| 708 | GTTCGGCTTGTGGTGTAATT | 1158 |
| 709 | GACACACAGCGTGACCTGAG | 1159 |
| 710 | GAGGCGACAGAAGGAGCTCA | 1160 |
| 711 | CTGCTCAGAGGCAGGGTGTA | 1161 |
| 712 | GGGAACCCGCTGCTCACCAC | 1162 |
| 713 | AGCCGCTGGATCCCGAAGGT | 1163 |
| 714 | GAGAAGACGACCCAGGTGAG | 1164 |
| 715 | TACTCCTGTCAGCTGATTGA | 1165 |
| 716 | CCACGGGCGGCAGGCGCCCG | 1166 |
| 717 | CCGGACAGCTTCCCCAAAGG | 1167 |
| 718 | CCCGCTGGACCTCACTCGGC | 1168 |
| 719 | AGGTTACAACATCATCAGGA | 1169 |
| 720 | GGTGCACACAGAACCATTAC | 1170 |
| 721 | CCGTGCTTTCTGGAACGAGT | 1171 |
| 722 | AGAGGTTCCTTGAGTCCTTT | 1172 |
| 723 | CCAGTGCCGCCTGCATTCGC | 1173 |
| 724 | GCGGCTGTTTTGCTATGCAG | 1174 |
| 725 | CCAAGTGGCTGGCCAACTTC | 1175 |
| 726 | GGAAGGGGACCGGTCCTGGG | 1176 |
| 727 | CAAGAACAGCATTGCATACA | 1177 |
| 728 | GAAGGAGCCAGAGGAAAAAC | 1178 |
| 729 | CACGTTGACCACGATGAGGA | 1179 |
| 730 | ATCAGTCATGCTGTCACAAC | 1180 |
| 731 | AACGGTGTAGCGGCGGGGGC | 1181 |
| 732 | GCCCATGGACAGGTTCGAGG | 1182 |
| 733 | GCAGCACAACACTGCTGCTG | 1183 |
| 734 | GCCGCTACTGCTCAGCCTGC | 1184 |
| 735 | CTGGAGTACCCGCATAGCCA | 1185 |
| 736 | AACGCTGTCCACCGTGGAGC | 1186 |
| 737 | GCGGGTAGAGGGTCTGCAGC | 1187 |
| 738 | TTGCTGATGTTGCTGGACCA | 1188 |
| 739 | CGGAAGATCTATGAGGAATG | 1189 |
| 740 | GCTTGTGACATGGGCATTCT | 1190 |
| 741 | TCTCAATATCCAGGAGTTGT | 1191 |
| 742 | AGTAGGCCTTGCCAAAGCCA | 1192 |
| 743 | CCCGGAGGCCGCAGAGGAGC | 1193 |
| 744 | CAGGTGGTGACCCCGGTGCC | 1194 |
| 745 | TCCTTTTATATTTAGGGGTA | 1195 |
| 746 | CCCAACTGAGAACCAAGAAG | 1196 |
| 747 | GCCGCTAGACACGGACGCGG | 1197 |
| 748 | CCTGTTTGTGCGGGAGCCCT | 1198 |
| 749 | CTGGACGTCCACCCGCTTGG | 1199 |
| 750 | GTACAGCCCGAGAGAGAGCC | 1200 |
| 751 | CGGGCTTCTTCTCCCTACTC | 1201 |
| 752 | ACCCGGATATCAAGAACTTT | 1202 |
| 753 | GAGCTGGTGCCTGGGGCTCC | 1203 |
| 754 | GCCCAACGTGAAGATGGCCC | 1204 |
| 755 | TTTTACAGAGATCCCTTCCG | 1205 |
| 756 | CACCGCGAGGCTAGGAGGAT | 1206 |
| 757 | AAAACGTTTACAGCTTCCAG | 1207 |
| 758 | CTCCTTCACCTGCTTGAGGT | 1208 |
| 759 | CGGACAGCTTCCCCAAAGGT | 1209 |
| 760 | CCACATGCACTCGCGTGTGG | 1210 |
| 761 | CGAGTCAGATCTGCAATCTG | 1211 |
| 762 | TTCCATGGGGGCCATGAGGT | 1212 |
| 763 | TACCTGGTCCTTCTCCTCAG | 1213 |
| 764 | CGATGGAGAGGCGTGAGCGC | 1214 |
| 765 | ACGGTGACCTCAAGGCTTCT | 1215 |
| 766 | CCGAGTCAGATCTGCAATCT | 1216 |
| 767 | CGTTGCAATCCCTTAAGCAT | 1217 |
| 768 | CGGAGGGCATCCGCATCTGC | 1218 |
| 769 | GATCCTGTCATCATCATCAA | 1219 |
| 770 | CTCATACTGTGGAATGCCTG | 1220 |
| 771 | GGAAGCAAACCGCAATTCTT | 1221 |
| 772 | AGGAGCAGCCACGCTCAGTG | 1222 |
| 773 | CGGAGGGGATGGCGCCTAGG | 1223 |
| 774 | CCGGGGCTGCCCCTGGACGC | 1224 |
| 775 | ATGTTCGACAGCGGAAACCC | 1225 |
| 776 | CGCGAGAAATCTCGAACCAG | 1226 |
| 777 | GCCCAATTCCTTTAGCAATG | 1227 |
| 778 | CCCGGGTCTGGGACAGGACC | 1228 |
| 779 | GCCCTGTGCCCTGCGAGCCA | 1229 |
| 780 | GACCGGAGGAGAACTACTGC | 1230 |
| 781 | TACAGTGTTCCTAAAAGGCA | 1231 |
| 782 | CTCACGCAGCTTGCGCAGGT | 1232 |
| 783 | AGCCCGACCCACATCAAGGC | 1233 |
| 784 | CCCTGCCTCGCGCATTCGGC | 1234 |
| 785 | TAGGCACGTCAGACCCGAAC | 1235 |
| 786 | TTTCAGCCTTAGAAATACAC | 1236 |
| 787 | CGTGACGAAGGCTATGAAAG | 1237 |
| 788 | CCAGCATCATCCCCCAGGTG | 1238 |
| 789 | TATCACTCTTGAGGTCTCTG | 1239 |
| 790 | CCCTGAGGCAGACGAGGCAC | 1240 |
| 791 | AAGCCGCTGGATCCCGAAGG | 1241 |
| 792 | AAACCGCAAGTTTGGCTTTT | 1242 |
| 793 | CGCACTGCCATCATTGCCCA | 1243 |
| 794 | CCAGGCAGGAGGCTCGGGTG | 1244 |
| 795 | GTGGAGGACACGCAAAGGAA | 1245 |
| 796 | GGCGTAGTCCTTCCCAGAGA | 1246 |
| 797 | CTACCTGTCCGGGGTGCTGC | 1247 |
| 798 | GCGCCACATGCACTCGCGTG | 1248 |
| 799 | GGAGGCACAGGGCATGGGTG | 1249 |
| 800 | CTTCCCGGCAGGACGCAGCA | 1250 |
| 801 | GCCTCTCTTGGACACAAAGC | 1251 |
| 802 | GGTAGCGGCCGGTGCCGTCT | 1252 |
| 803 | AGCTCCGTTTCGTGATGTTT | 1253 |
| 804 | CGGCAACGGTGTAGCGGCGG | 1254 |
| 805 | CTCTGGCAACCGCTGCCTGA | 1255 |
| 806 | CATTCGCACAGATGAGTACA | 1256 |
| 807 | AGCGGCGCCTCAAGCAGCAG | 1257 |
| 808 | ACCTCCACTGGGCCGACACT | 1258 |
| 809 | CAATCAATCACTTCAGAGAA | 1259 |
| 810 | GGCCGACCCGCTGGAGGCGC | 1260 |
| 811 | GAGCCCGACCCACATCAAGG | 1261 |
| 812 | ATTCGCACAGATGAGTACAT | 1262 |
| 813 | CTGCTCTCCGCCGCCTGCTG | 1263 |
| 814 | ATGCCGGAGTTCAGTTTGTT | 1264 |
| 815 | CCACCGCCTGCTGGTGACCC | 1265 |
| 816 | CCGGAGCTTCCGGCCCTCTG | 1266 |
| 817 | ACAGTGTTCCTAAAAGGCAC | 1267 |
| 818 | ACAACCCGGAGCGCTATGGC | 1268 |
| 819 | CACAACTCTGGGGGAAACCA | 1269 |
| 820 | AGCCGATTGAGACTAGTGAG | 1270 |
| 821 | CGGCACCCGTACCTCCGGGG | 1271 |
| 822 | GCAGAAGAACACCCTGGCGG | 1272 |
| 823 | AAACCGCCCGGGGCAGGTGC | 1273 |
| 824 | GCGCTCGGCCGCGCGCCTTG | 1274 |
| 825 | CAGTGCCCGGCCATGGGCTG | 1275 |
| 826 | CTCGGCAAAGAACGCTGGGA | 1276 |
| 827 | CCTCGATGAAGCACTCGTTG | 1277 |
| 828 | TAACAGCGGGTCAGGCACCG | 1278 |
| 829 | GTTTTCTACCTTCTTTTCCC | 1279 |
| 830 | CCGCCATCCGCGCGAGTACC | 1280 |
| 831 | GTCGAGCTGCAAGGGGATAG | 1281 |
| 832 | CGGAGCTTCCGGCCCTCTGG | 1282 |
| 833 | GAGTCTGGGCTCTGCAAACA | 1283 |
| 834 | TCCATAGCGGCAGACATTAA | 1284 |
| 835 | GGGCCTGATGGAGCCACCCC | 1285 |
| 836 | TTTCTGAGCAGTGGCTCCGA | 1286 |
| 837 | GCGGAGGCCCCGGAGAGGTG | 1287 |
| 838 | TGTTGTTGTCGAGCTGCAAG | 1288 |
| 839 | CCGGTACACGGGCGAGGGCA | 1289 |
| 840 | GACTACAACCCGGAGCGCTA | 1290 |
| 841 | TTCCCGCTGGGCTACTCGGA | 1291 |
| 842 | ATACGGGAAAAAGGCGTGGT | 1292 |
| 843 | GCAGGGCGTCACGGTCGGTA | 1293 |
| 844 | GAAGCTCCGCCACACTGTGA | 1294 |
| 845 | AAGGCCGCCCGGGGTAAGGT | 1295 |
| 846 | CCACACGGCTGAGACGTTCC | 1296 |
| 847 | GCACAGGGCATGGGTGAGGG | 1297 |
| 848 | TGCCTCTCTTGGACACAAAG | 1298 |
| 849 | GAGCCGGACACTGAAACCTT | 1299 |
| 850 | CCGTTGCAATCCCTTAAGCA | 1300 |
| 851 | CCTCGGGCGCACCCACGAGA | 1301 |
| 852 | CCCGGACTTCGAGAACGAGA | 1302 |
| 853 | CCACCCGAGGCAGGGGCGGC | 1303 |
| 854 | CCGGAGGTAGGACCCGGCGG | 1304 |
| 855 | AGCTACTGGGTAAGGGGGAC | 1305 |
| 856 | AAGAAAACCTACGCCATGGG | 1306 |
| 857 | CGACAGGGTACTTCAGGGTC | 1307 |
| 858 | CCTCTGTCAGGGAGCCCTCC | 1308 |
| 859 | CTCTACCCGGACCCTGCATA | 1309 |
| 860 | GGCTCACGATGAGTGCCTGG | 1310 |
| 861 | AACCCGCTGCTCACCACCGG | 1311 |
| 862 | TCAAACGTGCAGATGCCAAT | 1312 |
| 863 | GAAGCCGCCGAAGTCCCTGT | 1313 |
| 864 | GTCCAGTCAAAGGAGCAAAG | 1314 |
| 865 | CAAGCGCTCCTTCTTCGATG | 1315 |
| 866 | GGCCGCCGCCCAACGCCATC | 1316 |
| 867 | GGCATGCATCCGCTCATCAC | 1317 |
| 868 | TGCGGCATTTGTCCTGCTCC | 1318 |
| 869 | CTGATCTCAGGTACAGTTTC | 1319 |
| 870 | ACGTCCAGTCAAAGGAGCAA | 1320 |
| 871 | GTTGCCGGCGCGCCTCGCAG | 1321 |
| 872 | GCTCCGGAGGTAGGACCCGG | 1322 |
| 873 | CTCCGGAGGTAGGACCCGGC | 1323 |
| 874 | TCCGGGGGCCCGCCCAGATC | 1324 |
| 875 | GAGTAGGCCTTGCCAAAGCC | 1325 |
| 876 | GCGCGGCACCCGTACCTCCG | 1326 |
| 877 | CGATAGGCAGCCTGCACTGG | 1327 |
| 878 | GTGGGCCGTGCTGTGGGAGT | 1328 |
| 879 | CCGCTAGACACGGACGCGGC | 1329 |
| 880 | ATGGAGGTGACTGGGCCTAC | 1330 |
| 881 | GCGCCATTGGGCCGTGGTCC | 1331 |
| 882 | TGACCACCTGGCTTCCTACT | 1332 |
| 883 | ATAGATCCATCTGGTCCTGC | 1333 |
| 884 | TTCACTCTCTTAACATTCAG | 1334 |
| 885 | CTGCGACAACAACGGTTTTC | 1335 |
| 886 | CACCTACTCCACGAAGCGCC | 1336 |
| 887 | GGGGCAAAGGCTGTGCTCAA | 1337 |
| 888 | GCCGGACTCACCAGGACCAG | 1338 |
| 889 | CGCTGTCAGGTGCAAGCTCT | 1339 |
| 890 | GACCTATCCTAAGGTACGTG | 1340 |
| 891 | GAGTTCAAGTCCCCACCACA | 1341 |
| 892 | CTACAGGCGGCAGGCGCCCG | 1342 |
| 893 | CTGGCTAGATATGAGAAGCA | 1343 |
| 894 | GTGTCAGGGGCGGCCCACGA | 1344 |
| 895 | TGTAGCTACTGCTGCTGCTG | 1345 |
| 896 | CCTGTTTCATCTTGCTGGCA | 1346 |
| 897 | CTTGAGATTGCTGGGATCAC | 1347 |
| 898 | GCACCTACGTCTCCTGGTCG | 1348 |
| 899 | AAGGGATCCGCCTGGTCCTC | 1349 |
| 900 | GCACATATCCCAACAGCTCA | 1350 |
| 901 | AGATGCTGAGCCCGAAGCAG | 1351 |
| 902 | GACAGCTCCCACGCCTACAT | 1352 |
| 903 | ACCAGCCCCCCTGGGCCTCC | 1353 |
| 904 | TGTTGCTACTGCTGCTGGGC | 1354 |
| 905 | TCACGACTGGGATGAACCAC | 1355 |
| 906 | CGGCGCCCTATAGCTGCGCG | 1356 |
| 907 | CCTGCCAAGTGAGGTCCAGC | 1357 |
| 908 | ACTGATGCTCCTGGCAGCCC | 1358 |
| 909 | AGACTTCGGGCCAGGCTTGA | 1359 |
| 910 | CCTGAGGCGCCCCACGATGG | 1360 |
| 911 | GAAGCTGAGCGACGAGGGCA | 1361 |
| 912 | CTGAGATCTGGTTTGCAACT | 1362 |
| 913 | TGTGCAAAAGGGCCAGGCAG | 1363 |
| 914 | ACTCAGGGGTCGGCAGGCAG | 1364 |
| 915 | GTCGCTAGGCAAAGTGGTCA | 1365 |
| 916 | CTCCCATTCTCCCGCCATGT | 1366 |
| 917 | GTCTCAGTGCAGGATGAGGT | 1367 |
| 918 | CTACAAGCTGCCAGACGGGC | 1368 |
| 919 | TGGCCCAGCGGTTCAGCCAC | 1369 |
| 920 | CTTCTATCCAGGGGCGATGA | 1370 |
| 921 | CAATTAATCCCAAGGAACGA | 1371 |
| 922 | TTAGTTCAGCCACCTTCTAG | 1372 |
| 923 | GCTGCCCACCTGGCTGTGGA | 1373 |
| 924 | CCACACCCGGCCAATGTTGT | 1374 |
| 925 | CCTTCACAGCTCCTGAAAGG | 1375 |
| 926 | TCTTTCAGGCAGCCTCTTTG | 1376 |
| 927 | TTTGATCTCCCTGGTCCTGC | 1377 |
| 928 | TCTTCATCCAGTGCTGGTCC | 1378 |
| 929 | GCGGGAACTCTCGGGGCAGA | 1379 |
| 930 | GGTCTACGTTCAGCTGGCCC | 1380 |
| 931 | CCCTAGTCCTCCAGGCTTCC | 1381 |
| 932 | AGAGCTAGGGGGGCGGTGGC | 1382 |
| 933 | CCAGCTACCCCAGCTACTGG | 1383 |
| 934 | CCCGTCCTACTGGGGCATCA | 1384 |
| 935 | TCAGGGACTCTGTGGGGATG | 1385 |
| 936 | CCGGGACTTTCCAGGGCCCC | 1386 |
| 937 | GGATGAGCCCCCAGGTCCTG | 1387 |
| 938 | CGAGTAGGGGAGCCACCAGT | 1388 |
| 939 | CTATTGGGACCCAGCCAAAC | 1389 |
| 940 | GAGTCAGTCATCGCTAGGGA | 1390 |
| 941 | TCCCCAGAAACTGGAATCTG | 1391 |
| 942 | CATGTATCTCGACATCCACG | 1392 |
| 943 | CGCTGACTTTGTCTTCTACT | 1393 |
| 944 | ACGTGATCCCCCTGGACCCC | 1394 |
| 945 | CGCCATATACGTGGCCATCC | 1395 |
| 946 | TGGGGACCATGAGGTGGGGC | 1396 |
| 947 | GGAGCCCCAAGGCCCGCTGG | 1397 |
| 948 | TCTCTGAGGAACAAGACTCA | 1398 |
| 949 | GCTGTAGCCCCTGCCGCTCT | 1399 |
| 950 | CCCAGTGAAGAAGGCAAAAG | 1400 |
| 951 | AGCTATGTGATGAAGCAGGC | 1401 |
| 952 | TCTGACCCTCCTGGTCCCCC | 1402 |
| 953 | CACATCCTATCCCACCAGTT | 1403 |
| 954 | ACCATCCCCGGCCGCCTGCA | 1404 |
| 955 | CATCACACCCATCTTTGCCT | 1405 |
| 956 | AATCAGTGGGCCATGGTGAC | 1406 |
| 957 | CTTTGCATCCACACAGAGTC | 1407 |
| 958 | ACTAGCCCTCCAGGACCTGC | 1408 |
| 959 | TGCTGACCAACCAGGAGAGA | 1409 |
| 960 | AGCGATTCTCCAGGCAAGGA | 1410 |
| 961 | AATCAGGCTTCCCTGGCTTG | 1411 |
| 962 | CCTACCGCAGCCTGTTATAA | 1412 |
| 963 | CCACCAGCCCTCCCTGTGGT | 1413 |
| 964 | CTCATGGCTGAAGCTGTGTT | 1414 |
| 965 | TGCGGAGTCTCTCGGCCCCC | 1415 |
| 966 | CCACACCGGGGAGGTGGGCT | 1416 |
| 967 | ACAGTACAGCTCACTCAGTG | 1417 |
| 968 | CAGGTCAGGCCAGCTGGGGC | 1418 |
| 969 | GAGCAAGCCATGGCACATTG | 1419 |
| 970 | AGGCCTGGACTGTAGAGACA | 1420 |
| 971 | CTTGGGTCAGGCTGATTCAG | 1421 |
| 972 | TTCTCCCCAGTGGGGTCTGG | 1422 |
| 973 | CATTTAATCAGGGTCTTGAA | 1423 |
| 974 | CGGGCACAGTGCGGATGCGT | 1424 |
| 975 | CCTGACCCCAAGGGAGACCC | 1425 |
| 976 | CCTGAAGTGTCTGGACCAAA | 1426 |
| 977 | TTGGCTAGCAGCTGCTGCAG | 1427 |
| 978 | GCCTGAGGCCGTGGAGACAG | 1428 |
| 979 | TGGGTCATCGTCTTTGAAAA | 1429 |
| 980 | AGATCAGTTGTACCAGATGC | 1430 |
| 981 | GTTGAGAGGCTATGTGTGAC | 1431 |
| 982 | CTCACAGGTCCCAAAACGGT | 1432 |
| 983 | GATGTTCTAGGAAGCTCGGC | 1433 |
| 984 | GGACCTAGGCGAGGCAGTAG | 1434 |
| 985 | ACACAGAGTGCTGAGCGGTG | 1435 |
| 986 | CCCTCACTGGCGGCTTTTCC | 1436 |
| 987 | CCAGATTTGAAAGGAAAACG | 1437 |
| 988 | GCTGAGCCCCAAGGTCCTCC | 1438 |
| 989 | GTCGGAGGCCCCTGGCAAGG | 1439 |
| 990 | ACAGGACCCCGCTGGCCAGG | 1440 |
| 991 | TTGTCATGAAGATGCACAAT | 1441 |
| 992 | TCATCAAGAAGGTGCCTCTT | 1442 |
| 993 | CCTAGGAGAAAGGGCTTGGA | 1443 |
| 994 | GCTAGCGGTGATAGTCAGCC | 1444 |
| 995 | GAGAGCTGATAGAAGACTCT | 1445 |
| 996 | CCTCAACTGCCAGACTGCAG | 1446 |
| 997 | TGCTAGGTGACCTTGGCCTC | 1447 |
| 998 | CGAGTCTAGCCATCCGCTGT | 1448 |
| 999 | CCCAGCATCGGCTCCGTCTC | 1449 |
| 1000 | GGGGACGCAGGCCTGGAGAA | 1450 |
| 1001 | TCGCTAGCGGCTTTTCCTGG | 1451 |
| 1002 | CCTGGCAGCCTATGAGTCCT | 1452 |
| 1003 | ACCTCAGGCACGGCAGTGGA | 1453 |
| 1004 | TCCTGTAGGAGTTTGCCAGC | 1454 |
| 1005 | TAAAGGGCATGCTCATCTCC | 1455 |
| 1006 | GAAGAGATCGCCTGGTGCCC | 1456 |
| 1007 | AAAGACCTCCGAGGAATCCC | 1457 |
| 1008 | GCTGCATGTCTCTGCGTCCA | 1458 |
| 1009 | CACTATCTTGCCCCAGGCTC | 1459 |
| 1010 | TACCTTAACAAGCTCCCGTG | 1460 |
| 1011 | CGTGCAGGAAAGAGCAGTGG | 1461 |
| 1012 | TCTAGCTGAGATCCGGGAGA | 1462 |
| 1013 | CATCAGGGACGCCCAGGCTA | 1463 |
| 1014 | CCCTTCGAGCTGGTAGCGCC | 1464 |
| 1015 | AGGCAGAAACCAGTGCAAGC | 1465 |
| 1016 | TACCACCTGCTGGAGGGGTC | 1466 |
| 1017 | TCAGGTCATTCCATGGGGGA | 1467 |
| 1018 | CCGGATGATATGGGAAAGAA | 1468 |
| 1019 | CATCAAGTGGCATATCTATC | 1469 |
| 1020 | GCTGAAGAAAAAGGCAACAA | 1470 |
| 1021 | CCCAGTTCTTCTGCACATAT | 1471 |
| 1022 | GCACCAAGAACACGCCCCAG | 1472 |
| 1023 | ATTTTAAGGGTCAGGGTTTG | 1473 |
| 1024 | GAATAAGATCGAGGACTTGA | 1474 |
| 1025 | GCTCAGAATGGCTGGGTCTC | 1475 |
| 1026 | GGTCAAGGGACCGGAATTTG | 1476 |
| 1027 | CAAGGACAGCTACTGGACGC | 1477 |
| 1028 | CCACGCCAGGGAGGTGGGCT | 1478 |
| 1029 | GTGCCAGCTACAGGGAAGGA | 1479 |
| 1030 | ACCAGACATGCCAGGTCCTA | 1480 |
| 1031 | GCCAATCCTATTCAGGGCCA | 1481 |
| 1032 | AATTCAGTGAATGTAGTATT | 1482 |
| 1033 | TGCTCAGTACATGGTGCTGA | 1483 |
| 1034 | TGCACTCAAGTCTGCACATC | 1484 |
| 1035 | CCTGGGAGCATTGGAGATTT | 1485 |
| 1036 | CAGAGGTCTCCAGGTTTGCC | 1486 |
| 1037 | ATGTTGCTATTGCTGCTGTT | 1487 |
| 1038 | ACTCAAGGTACTGGCGCTGG | 1488 |
| 1039 | GCCCAATGTTATCGAGATGA | 1489 |
| 1040 | AATTACTTTTGCCTAGTGCT | 1490 |
| 1041 | CGAGCAACTCCGCATCCCTG | 1491 |
| 1042 | GAAATAGCTGAGCATCAATG | 1492 |
| 1043 | CACCGACCCGCAGGAGACTG | 1493 |
| 1044 | CAAGACCAACAGGCCTTTCC | 1494 |
| 1045 | ACCTACTCCCCCAGGAACTC | 1495 |
| 1046 | TGACTACAGCTTGTACCCCC | 1496 |
| 1047 | CCCCGCCAAGTTCACCCCTG | 1497 |
| 1048 | GTTCTAGAACATTAACCCGA | 1498 |
| 1049 | ATGATGTCCCTGCAGCTGCG | 1499 |
| 1050 | ATCAGCTGGAGTTGTTGTTC | 1500 |
| 1051 | CCAGATCCTCCTGGGCCATC | 1501 |
| 1052 | CCCAGGCTCCCAGGCAGGGC | 1502 |
| 1053 | CCTCATCCCAGGCAAAAATG | 1503 |
| 1054 | GAAGGCCGACCGGTCGGCGA | 1504 |
| 1055 | CCCTACAGGGCCCCAGGCCC | 1505 |
| 1056 | CTCCCAGGTCATCTCTGCCA | 1506 |
| 1057 | ACGTGCAGCGGAAGGCTGAT | 1507 |
| 1058 | GTTCCCACGCATACACGTCT | 1508 |
| 1059 | CCTTAGTCTGGCCAGTTCTT | 1509 |
| 1060 | CTTCTAGAGGGTGCTGTTCA | 1510 |
| 1061 | GAGCCGGACAACCCGGGCAA | 1511 |
| 1062 | CCCGCCACGCCAGGAATGTC | 1512 |
| 1063 | CCTCAGGTGGCCCAACGGCC | 1513 |
| 1064 | ACCACCTAGGCCAGCTTGAA | 1514 |
| 1065 | TTGTGATGGCCACAACCATG | 1515 |
| 1066 | CGTGGATCACGCCTGGGGGC | 1516 |
| 1067 | GCTAGCTCCAAAGGAGAGAG | 1517 |
| 1068 | GGTTCCCCAGATGATTCTGG | 1518 |
| 1069 | GCTACTGCAACACAGCCACC | 1519 |
| 1070 | GGACTACATCTGTGGCTGGA | 1520 |
| 1071 | CCAAGTCCTCAGGGTCTTCT | 1521 |
| 1072 | GTTGTAGCGGAGAAGGGCAG | 1522 |
| 1073 | GCAGCACCTGCACATGCTGG | 1523 |
| 1074 | CTGAGGCTGCCCCTGGACGC | 1524 |
| 1075 | CTCTTAGTGGCTCTGCTGAT | 1525 |
| 1076 | AATGATGCTCAGGGACCTCC | 1526 |
| 1077 | GAATGATGAAACTGGACCTC | 1527 |
| 1078 | GCCGCTAGACAATGGGAGTG | 1528 |
| 1079 | AGACCTCTAGAAGTCCTTGA | 1529 |
| 1080 | CCTTTAGAGTAGCTGCCTGA | 1530 |
| 1081 | GCGGCTACACCTGTCATGGG | 1531 |
| 1082 | ACTGCTACCCATATATCCAG | 1532 |
| 1083 | CCTCAAGGACCGGCAGGCCC | 1533 |
| 1084 | GTTCGAGGAGCTGGTGGCAG | 1534 |
| 1085 | GCCTCAGCTCTGCCCTCAAC | 1535 |
| 1086 | GGCCTGTAGGGTTTCATTAA | 1536 |
| 1087 | GTTCCTGCAGTTGTTCTCCA | 1537 |
| 1088 | AAGCACATGAGGCATTCTGG | 1538 |
| 1089 | GGGCTAAGTGGGGTACACGC | 1539 |
| 1090 | CAAAATACTTAGGAGAAAAA | 1540 |
| 1091 | GTCATCTAAGACCCACTCAC | 1541 |
| 1092 | GAGGGCTAAGCCAGCAGGTG | 1542 |
| 1093 | AGATACCCCTCCATCCGGAG | 1543 |
| 1094 | CCGCCTCCACGTCGCCTCCA | 1544 |
| 1095 | CAACTCACTTCAGCTCCTCA | 1545 |
| 1096 | TCTATGTCCCCCGAAGGACA | 1546 |
| 1097 | CCGTCATGTGGGTCCTGAAT | 1547 |
| 1098 | TTTCTACTTCTGGAACAGCT | 1548 |
| 1099 | TCCTGCAGCCCAGGCAGGCC | 1549 |
| 1100 | CGTAGTGAAAATGGCTCTCC | 1550 |
| 1101 | AATATGCCAATGCAAGTCCC | 1551 |
| 1102 | CTCCCCTCAAGGATCACGTT | 1552 |
| 1103 | TGCAGCCCACACCTGCCGCC | 1553 |
| 1104 | AACAGTGTTGTTGGTCCCAC | 1554 |
| 1105 | GATCTACAGGCTCAGGCACC | 1555 |
| 1106 | GTGGCAGCCAGCAATAGGCA | 1556 |
| 1107 | GGCCTACTTTGGAGGTGATG | 1557 |
| 1108 | GATCAGGGGTGTCCTCGGGG | 1558 |
| 1109 | CCATAGTTTGGACTGGATAT | 1559 |
| 1110 | GCAACATCTGCACTCTCGTG | 1560 |
| 1111 | AGCAGACTGGATGGGAAACC | 1561 |
| 1112 | GAACATCCTGGGGACGACTC | 1562 |
| 1113 | CTTCTCACCTGCTGGATGGA | 1563 |
| 1114 | GGGTTAGAATGACCCAGATA | 1564 |
| 1115 | CCCTGATCCCGAAGGAGGAA | 1565 |
| 1116 | GCGCTACCTCGAGGCCTTGG | 1566 |
| 1117 | CGTGATGAGGTCGGTCCTGC | 1567 |
| 1118 | TGCGGCAGGACACTTGTGCC | 1568 |
| 1119 | TGCACGAGCTCCTCCGGCCC | 1569 |
| 1120 | GTCAGTGTACCAGGATGCAG | 1570 |
| 1121 | TCACTGGGCGACGGGCCCCT | 1571 |
| 1122 | TCCAGGCGGCAAGAGAGAAG | 1572 |
| 1123 | GCTTTAAGGCTTGCCCAAGG | 1573 |
| 1124 | GAGTCCTACTTGGCCAGGCC | 1574 |
| 1125 | AGTAGCCCTGCTGGAGTCCG | 1575 |
| 1126 | CCCAGTCCTGCTGGTTCCCG | 1576 |
| 1127 | CCAAGGCCCCCTGGCCATCC | 1577 |
| 1128 | TGCTTAGGGTCCAGCCATTC | 1578 |
| 1129 | TTTCAACATGGCCCATGGGC | 1579 |
| 1130 | CTGCACTGGGGAAGGCCCGG | 1580 |
| 1131 | AGTCTCACTCCCCCTCCTGC | 1581 |
| 1132 | GGTCAGGCCAGTGCCCATGG | 1582 |
| 1133 | TATGATGGTGGTTTTCAAAC | 1583 |
| 1134 | GCCTCAGCACACAAAGTGGT | 1584 |
| 1135 | AGGTCCACGCCCGTCAGCTG | 1585 |
| 1136 | CAGGGCATGATGGTGGGCAT | 1586 |
| 1137 | ATGTGTGTGAATCCTGGAGG | 1587 |
| 1138 | CCGCTAGCCCACATGCACAG | 1588 |
| 1139 | CCAGACCCTCCCGGTCCCCC | 1589 |
| 1140 | TCCTACAGCGCCACACCGCT | 1590 |
| 1141 | GAACTATTCATACTGGAAGC | 1591 |
| 1142 | CTTACCGGACGTCAGTGATC | 1592 |
| 1143 | CCTAGAGCTCCTGGCGAGAG | 1593 |
| 1144 | GCTGACCCTCCAGGCTTCCC | 1594 |
| 1145 | CCCTACTGTCCCACATGGGC | 1595 |
| 1146 | CAGGCAAGCCTGGCTGCTGG | 1596 |
| 1147 | CTCTTAGCCGTGCGTCAGGA | 1597 |
| 1148 | TGGCCATGAGGAACACCACG | 1598 |
| 1149 | GTCAGCCTCGCTTGACCCTC | 1599 |
| 1150 | GGAGTCCTACAGGCCTGGGC | 1600 |
| 1151 | CCAGATCCCAGCGGTTCTCC | 1601 |
| 1152 | TCTAGTCAGGAGGATGGCAA | 1602 |
| 1153 | TTCCCTGAGCTGTACAAACA | 1603 |
| 1154 | CCCAGCTGCTGTGGGTCAGA | 1604 |
| 1155 | AAATTCTACTGGCTTGTATT | 1605 |
| 1156 | GGGGTCAGGGGACAGCCTTG | 1606 |
| 1157 | CGGAGAGAGAAAGGAGAACG | 1607 |
| 1158 | TGTGGAGGCTGCAGCTACAC | 1608 |
| 1159 | GTCTCATGCCTGCTCGTGGC | 1609 |
| 1160 | ACAGCTCTAAGAGTGAAGAC | 1610 |
| 1161 | GGTATATGGCAGCTTTGGCC | 1611 |
| 1162 | AGAATTAGATCTGGATCATT | 1612 |
| 1163 | GTGGTTAAGGGGACAGCTGC | 1613 |
| 1164 | AAAAGGGCTCCAGGACCCAA | 1614 |
| 1165 | ATTAGTCCACCATGTTCTTC | 1615 |
| 1166 | GAACTAGCCATCAATACTGT | 1616 |
| 1167 | CACAGACTGCCAGGCTATCT | 1617 |
| 1168 | CACCTACAAGTCCCTGCCCA | 1618 |
| 1169 | TGGCCCACATGTTCTACCAC | 1619 |
| 1170 | GGACTCAGTTGGCAAATCGG | 1620 |
| 1171 | GGCTCTACAGTGGGCCGGTG | 1621 |
| 1172 | GGGCTGATTTGCCATCCGAG | 1622 |
| 1173 | GGAGTAGGAGTACAGAGACG | 1623 |
| 1174 | GGTGCAGGGCGGGGTGGAGG | 1624 |
| 1175 | CAAGCAGAACCGGCCACCCC | 1625 |
| 1176 | CCTGATGCCCCTGGCGCTCC | 1626 |
| 1177 | GCTGGAGGCTTAGGCTTCCC | 1627 |
| 1178 | CCTGAAGAAAGAGGAGGTCT | 1628 |
| 1179 | GTGGACTGTGCTGTGGGAGT | 1629 |
| 1180 | ACATTTCATCCATCCTCTCC | 1630 |
| 1181 | TGTTCACTCAGCAGCATTTG | 1631 |
| 1182 | TGACTTGCACAGGTAGGGGG | 1632 |
| 1183 | AGGGGTAAAGGGTCAAAGCG | 1633 |
| 1184 | CTCCTAAGAAGGGGACATTG | 1634 |
| 1185 | GCACCATGGGCGTCTTCACA | 1635 |
| 1186 | TCCTGATGGTAAAGGCGAAA | 1636 |
| 1187 | ATGTTGTGCATGGTCACTGG | 1637 |
| 1188 | TGTTCCAAAAGTCTGAGTTG | 1638 |
| 1189 | CCATGAGGGAACCAGGTGAG | 1639 |
| 1190 | CACGTGAATGAAGCATACGA | 1640 |
| 1191 | GCAACAAGTTGGGTGGCTGG | 1641 |
| 1192 | CCAAGCCTCTCCGGCCCCGT | 1642 |
| 1193 | CCTGATCTCAGAGGTGAAAT | 1643 |
| 1194 | GTCTTAGTCCCACTGGAAGA | 1644 |
| 1195 | CCTCAGGGACCAGCAGGACC | 1645 |
| 1196 | TTCAGTCTCGTGTATCTTCT | 1646 |
| 1197 | ACTATGTGCAGTGGAAGACT | 1647 |
| 1198 | GCCAGCCCTACTTGTTCTCG | 1648 |
| 1199 | GTGAGTTCTGCTGCATCACC | 1649 |
| 1200 | ATCACAAGCCCCAATGGCTG | 1650 |
| 1201 | TCCCCTCCATGTCTGGGTAC | 1651 |
| 1202 | TCTGCAGGATGCCGTTGTCC | 1652 |
| 1203 | GATGACAGAGGAGCTGAAGA | 1653 |
| 1204 | CCAGATGGTGGATGTGGAAC | 1654 |
| 1205 | CCATGCCTAGTACCAGGCTA | 1655 |
| 1206 | GTGGTGAATGGACATGATGA | 1656 |
| 1207 | GCTGAAAGTCGTGGTGATGG | 1657 |
| 1208 | GCAGCTATGTCCACACCTGG | 1658 |
| 1209 | GTAGCGGCAGTGGAACTCAC | 1659 |
| 1210 | CCTAGTCCTGCAGTCAAAAG | 1660 |
| 1211 | TCATCATGGCGGGCCTTCGA | 1661 |
| 1212 | GAGTATGGGGTATGCCGTTG | 1662 |
| 1213 | AGGGTTATCCCTTGGCATAG | 1663 |
| 1214 | GCTAGTTCAACTTCTCCATG | 1664 |
| 1215 | CCCTAGATCCCCTGGTGCTA | 1665 |
| 1216 | ACAGCATCCGGCCATGGCCC | 1666 |
| 1217 | AGCAGCAGCCTTTGCCCCCG | 1667 |
| 1218 | CCTGATTTACCTGGCACTCC | 1668 |
| 1219 | ACGCGATCATCCCTTCTTTC | 1669 |
| 1220 | CCAGGCAGATATCTGTCAGA | 1670 |
| 1221 | CCCCTAGGGGCCGGGAGCAC | 1671 |
| 1222 | GGGCCACCAGGTCCCGAGGG | 1672 |
| 1223 | GGGGCTAGGGTTGGACAAGC | 1673 |
| 1224 | GCGCTACCGTAACGGCACAT | 1674 |
| 1225 | GTGGCAATCATTTCCCTAAA | 1675 |
| 1226 | TGCTGTCACTTCCTTCGAGA | 1676 |
| 1227 | CCTGAGCCATCTGGTCCCCG | 1677 |
| 1228 | AAAGCAGGCCCTCCGCTGGC | 1678 |
| 1229 | CCCTTAGGCCCCAGGCCGAG | 1679 |
| 1230 | CAGGCTACCCTTGGAGGTCG | 1680 |
| 1231 | CTGCTACCACAGCTTCTCCT | 1681 |
| 1232 | CCAGGGACGCCCTGGCTACC | 1682 |
| 1233 | AGCATCCATTATTGCAGATC | 1683 |
| 1234 | AACTGATCCTTATACCTGTT | 1684 |
| 1235 | TGGCTAGCAGTGCAGGGACA | 1685 |
| 1236 | TGTCACTGGGCAAAGTGGTC | 1686 |
| 1237 | CGTGGAGAAAGAGGTAGGCC | 1687 |
| 1238 | AAGCAGCTCCAGGCTTTCCA | 1688 |
| 1239 | CAACTAGAACTCCCGTAATT | 1689 |
| 1240 | AATGACTTACCTGGGAACCC | 1690 |
| 1241 | GCTGAGCCCTGGCGCTGCTT | 1691 |
| 1242 | AGTGGAGCAGCAGCAAGCGT | 1692 |
| 1243 | CTTATGGGTCTGGCAGGCTG | 1693 |
| 1244 | CTCCTAGCTGGAGCTGCACC | 1694 |
| 1245 | CCAGCTTCACCAGGTCTCGA | 1695 |
| 1246 | CTGACCGGGAGGCCGCGCTG | 1696 |
| 1247 | CACTGGCCATAAGATTATTG | 1697 |
| 1248 | CCATTAGTAGGCTCGGCGCT | 1698 |
| 1249 | TTTGTTCCCAGAGCTCTACC | 1699 |
| 1250 | ACAGCGACCAGCTGGGGCAG | 1700 |
| 1251 | CCGCCGCTAGTAGTACCCGC | 1701 |
| 1252 | ATGAGTGTCCGGAGCAGCTG | 1702 |
| 1253 | GCAGGATCAGGTTCACTCCT | 1703 |
| 1254 | GGCCGTGGACGAGTTCGACG | 1704 |
| 1255 | TAGCAGCCGGTGAAGTGGGC | 1705 |
| 1256 | CGGCCTATGTAGTTGAAGCT | 1706 |
| 1257 | CCACATGTCCTGTAAGTACT | 1707 |
| 1258 | CGGACGCCCGGCTGCTCCTG | 1708 |
| 1259 | GGTAGGTTATGGTCTTCAAA | 1709 |
| 1260 | AAGGCCACCTGGGGTAAGGT | 1710 |
| 1261 | GTTAGCCGAACTGGAGAAGT | 1711 |
| 1262 | CTTAGCTGGGGCTGCGGGAG | 1712 |
| 1263 | GGCCCTCATTCACCCTGGGT | 1713 |
| 1264 | GTTCTAGATGTCCACCCGCT | 1714 |
| 1265 | GCCCCAGCCTAAGCAGCGCG | 1715 |
| 1266 | GCCCAGGTTGGAAGAAGCTG | 1716 |
| 1267 | GAGCTATCGCACATCCAGAT | 1717 |
| 1268 | CGTAGAGAACAGGGCCTCCC | 1718 |
| 1269 | TGGCTATCAGTTGCAAAAAA | 1719 |
| 1270 | ATCCCCATGGGGAAAGAGGT | 1720 |
| 1271 | GTGTCAGCCCTTGGTGTCCA | 1721 |
| 1272 | AGAGATGAAATTGGTAACCC | 1722 |
| 1273 | CAGACAGGCAGGCCCATCAG | 1723 |
| 1274 | CCTAGCGCAGCTGGTACCAG | 1724 |
| 1275 | GCCTCAACTCAGCTGCTCAA | 1725 |
| 1276 | CCATGACTGGACGGTAAGGG | 1726 |
| 1277 | GGGTTTACAGGCGTGTTTTA | 1727 |
| 1278 | CATCATGCCTCCATCATTTC | 1728 |
| 1279 | CCGGCTTCCACCGCTTCCGC | 1729 |
| 1280 | CCGTCATGCTGTTGCTGAGC | 1730 |
| 1281 | CTCCATGTCCCAGGTCACGC | 1731 |
| 1282 | TTGGCCTACTCAAAGTTACC | 1732 |
| 1283 | CCTAGTGGTAAAGGAGAAAG | 1733 |
| 1284 | CTGCTACTCGGCGATGCGCT | 1734 |
| 1285 | CCTCAACGCCGTTCGGGCAC | 1735 |
| 1286 | CCGAGACCCCACACACCTGC | 1736 |
| 1287 | ATCATCCCATAAGCCCCACT | 1737 |
| 1288 | CAGTATTTTCATGGATAGGA | 1738 |
| 1289 | TCAGGCCACCCTCATCTGCC | 1739 |
| 1290 | CAGGGCCTAGGAGAAGTCCC | 1740 |
| 1291 | CTCGGGGGACGGGGACAGCG | 1741 |
| 1292 | TCTCTAGGGAACAAGACTCA | 1742 |
| 1293 | GGGGCAATCATCTCCCTCCT | 1743 |
| 1294 | CCACTAGATGCGCTCTTTGA | 1744 |
| 1295 | GATGAATGGCTGCAGGCTGG | 1745 |
| 1296 | GTCGATCCAGCTGGAAAGAG | 1746 |
| 1297 | GGGGATGAGTGTGTTGTTGA | 1747 |
| 1298 | GTTAGAAGAGTGCTTGGACT | 1748 |
| 1299 | ATGGTACGGAGGCCCTGGAG | 1749 |
| 1300 | CGCGTCTAGGATGCTTACAC | 1750 |
| 1301 | CAGCCACTCACTGTTTCTAT | 1751 |
| 1302 | CCCAATGGGCGGTTTGTCAT | 1752 |
| 1303 | CCTCAGGCACCAGGGAAGCC | 1753 |
| 1304 | CCCCTGGCTACGGGGGAATG | 1754 |
| 1305 | GCAATATGTGTGGCCACTTG | 1755 |
| 1306 | ACCTCACTCTCCAGCCTTGC | 1756 |
| 1307 | ACTGACTGGGGAGAAACCCA | 1757 |
| 1308 | AAGTGACTGGCCAACTTCTG | 1758 |
| 1309 | GTTTAGGGGTAAGTCCGGCA | 1759 |
| 1310 | TCCACTTGAAGAAGCCGACC | 1760 |
| 1311 | TTCTCACTTTTCGACAGGAG | 1761 |
| 1312 | CAGGCAGGCAGCCAGGATCA | 1762 |
| 1313 | AGCCCTACGAGGAGGATTCG | 1763 |
| 1314 | GCTACCTCTGGTACCAGTCA | 1764 |
| 1315 | AGCAAGGGTCCCCTACCCAC | 1765 |
| 1316 | TCATAGCTATCACTATGGAG | 1766 |
| 1317 | GCGTAGAGCCGCGATAACCC | 1767 |
| 1318 | CCATCGTGCTGTTGCTGAGC | 1768 |
| 1319 | TCTGGAACTGCTGATGGCTC | 1769 |
| 1320 | ATTTTAAGGTTCAACCCCTT | 1770 |
| 1321 | ACCACCATGTCTGACACCTT | 1771 |
| 1322 | CACTCAGATTCTGTGTCCAA | 1772 |
| 1323 | AATGACCCACAACACTGAGC | 1773 |
| 1324 | TTCTGCACATGGCGGTCACT | 1774 |
| 1325 | CTACTACCAGTGCAAGCTGG | 1775 |
| 1326 | GTAACGACAGACTTCTCCTC | 1776 |
| 1327 | TCGCAGAAAACGGTGCGCAC | 1777 |
| 1328 | CGAGAACGGCCAGGACTTCC | 1778 |
| 1329 | GTATGCTAGCTTTGCGAGTT | 1779 |
| 1330 | CCCAGGCCTTCCGGCCTTGC | 1780 |
| 1331 | CGGTGCAGAAGAGGGACTGG | 1781 |
| 1332 | CAGGAGCAGCACCTGGAAGC | 1782 |
| 1333 | TCATTGATGGTGATGTCCTC | 1783 |
| 1334 | ACAAGACATTCGTGGCGATA | 1784 |
| 1335 | AAAGCACTACACTGAAGACC | 1785 |
| 1336 | GGCCGGCAACGTCTTTAGCT | 1786 |
| 1337 | CAACTAAAAGAGTGCCAGCC | 1787 |
| 1338 | CGCAGGGCATCAACTGGGAG | 1788 |
| 1339 | AATCTCACACACGAAATTGT | 1789 |
| 1340 | CGGTTACAGTCACTGATAGT | 1790 |
| 1341 | GAACTAGTAGATGCCGTTCA | 1791 |
| 1342 | ACCACCACCGTGGACGTCGT | 1792 |
| 1343 | GGCCAGGCTGCTGGGGCTGC | 1793 |
| 1344 | AGGTCCAGCACATCTTCTCC | 1794 |
| 1345 | CCTGAATGGCCAGGCCTGAA | 1795 |
| 1346 | AGGCTGATACTTCTACATTC | 1796 |
| 1347 | CCTAGGAACGATGGTCCCCC | 1797 |
| 1348 | AACGATGCTCCTGGTGAAGC | 1798 |
| 1349 | GAATGACATCAACCTGGCAC | 1799 |
| 1350 | ACAGATGAACGTGGAGCTGC | 1800 |
| 1351 | AGGCAATGTACATAAATCTG | 1801 |
| 1352 | ATGGAGGTAAAAGGGACTAT | 1802 |
| 1353 | CTTATTCGATGAAGCTGGCC | 1803 |
| 1354 | TGCAGCCGCAGATCCCGATC | 1804 |
| 1355 | TCTCTCTAAAATCACTGAGC | 1805 |
| 1356 | GGACTACAATAGATTCCCGC | 1806 |
| 1357 | TCCCAAATCTCCCTAGAACG | 1807 |
| 1358 | TGAGGCTGCGGCAGCCCGCC | 1808 |
| 1359 | TACTTCAACACAGTGCCACA | 1809 |
| 1360 | CCCAGAAGTCCAGGAGGACC | 1810 |
| 1361 | TGTCACCCTGACTGCGGGCC | 1811 |
| 1362 | CCTGATCATCCAGGCCCACC | 1812 |
| 1363 | TCACTGACAGACAGTGGCCC | 1813 |
| 1364 | GCGAGCAGCACGAGTTTGCG | 1814 |
| 1365 | TTCCTACTTGGAGTGATTTC | 1815 |
| 1366 | CCAGTGAAAGCACTATTGAC | 1816 |
| 1367 | CTAGCCAACATTGTTTTGTG | 1817 |
| 1368 | TGGCTCAAACCAGAGGCTTC | 1818 |
| 1369 | GAACAATCTACAAGGGAAAG | 1819 |
| 1370 | GACAGAGGGAGTACTCGGCG | 1820 |
| 1371 | TCCGGAGCAGCATCCACACA | 1821 |
| 1372 | GAAGAGGCAGCTGGTTCCAC | 1822 |
| 1373 | GGACTACAGAGTAGTCCGGC | 1823 |
| 1374 | CCCACTCCTGGATCAAATAA | 1824 |
| 1375 | AAGGACGACAGAGGTTTGCC | 1825 |
| 1376 | TGGCCTGACACGTTCTCATG | 1826 |
| 1377 | TGCTTAAGAGAGGTAGAAGG | 1827 |
| 1378 | GTTTCAATAAGCCCGACGGA | 1828 |
| 1379 | CAAAGGACACAGAGCCAAAT | 1829 |
| 1380 | TGCTCCCAGGCATACACATC | 1830 |
| 1381 | TGACTGATGTAAATACAATG | 1831 |
| 1382 | TGACCTTATATGTTGGTGTG | 1832 |
| 1383 | CTCAGCTGACCGATCGCTTC | 1833 |
| 1384 | GCTGAACCAAATGGCATCCC | 1834 |
| 1385 | TGGTATCCCTTTGGATTTGA | 1835 |
| 1386 | GCATCCACTATCCCAGTAAG | 1836 |
| 1387 | CAAACCACCCACTGGGCTGC | 1837 |
| 1388 | GGTCCCCAGGGCCTCAAGGT | 1838 |
| 1389 | CGCAGGCTGAAGGGCGACCG | 1839 |
| 1390 | AGCCAAGTCAGCGCTGCTCG | 1840 |
| 1391 | TTAGTAATACTTGTGGGCCA | 1841 |
| 1392 | CCTGATGAACCTGGGCAAGC | 1842 |
| 1393 | CACGGACGTGGCGGCCGCCG | 1843 |
| 1394 | AGAACTACTCCACAGGGTTC | 1844 |
| 1395 | GTCCAAGGCTTGCAGCTGCC | 1845 |
| 1396 | AATAATGCCAGAGCATTAGA | 1846 |
| 1397 | CCAGAGGTGAAGGGTCAAAG | 1847 |
| 1398 | TACTGGAGAACGGCAACCAC | 1848 |
| 1399 | GGACACCATTCAGCGGACTG | 1849 |
| 1400 | ACCTAGTGTGGTTGGTGCTG | 1850 |
| 1401 | AGGCCCAAGATGAGCACACA | 1851 |
| 1402 | GGTTCTAGCACATGGAGATG | 1852 |
| 1403 | GGTGGCATGGCTCGGGCCTG | 1853 |
| 1404 | AACCAGATGGACAGCCACAC | 1854 |
| 1405 | CCATCTCAACCTGGGGCTCC | 1855 |
| 1406 | TGGTCTGCTAGATGGACAGC | 1856 |
| 1407 | TGCGTCAGATTCTTTCTAGA | 1857 |
| 1408 | TAGTTCTCACTCAGTGGACA | 1858 |
| 1409 | TCTCAACAGCCTGTGGGAGA | 1859 |
| 1410 | AGCAGCAACGATTGTTGGTG | 1860 |
| 1411 | TTCTTATTGAGGGCTATGCC | 1861 |
| 1412 | AGGGCCAACACAGTGGAGGG | 1862 |
| 1413 | CCCAGGGACCTCGGACCTGT | 1863 |
| 1414 | ACCAACCACCACTTTCTGAT | 1864 |
| 1415 | AAGGGGCAGGTGACAGGCTG | 1865 |
| 1416 | CCCCAAGTTCCTGAGATACC | 1866 |
| 1417 | GAAAGTGGAATCACACTGAG | 1867 |
| 1418 | CCTGAAATTCCAGGACCTCC | 1868 |
| 1419 | GCCTAAGTCCAGGGTTCAGG | 1869 |
| 1420 | GCAAAGGTACCAGCTTAGAC | 1870 |
| 1421 | GCCGATCCTACTGGTCCTAT | 1871 |
| 1422 | CTCAAGTATGGCTATGATGC | 1872 |
| 1423 | GACGCACATCAATGCCACCC | 1873 |
| 1424 | GAGGGAGAACGGGCCACAGC | 1874 |
| 1425 | AGAGATGACCAAGGACGTGA | 1875 |
| 1426 | GCCAGTGCTACTGGTGCCAG | 1876 |
| 1427 | GCTGATCGCCCTGGGGAGCC | 1877 |
| 1428 | TCAGGCAGCCTTGGGCTGCT | 1878 |
| 1429 | GTACTAGAAGATGCAGTCCC | 1879 |
| 1430 | AGCGTTATATATTCTCTGTG | 1880 |
| 1431 | AAAGATGAAAGAGGATTTCC | 1881 |
| 1432 | AAGTCCGAGCAACGGGGCCG | 1882 |
| 1433 | GGACTATGACCAGCATAGAA | 1883 |
| 1434 | GGGCCACTTGTTGGCTGGCT | 1884 |
| 1435 | GGCCCAAGAGAGTCTTGCCC | 1885 |
| 1436 | ACGTGAAACATCTCGTTCGC | 1886 |
| 1437 | AGTCACAGATCTTCACTTCC | 1887 |
| 1438 | AGTCAGAGATGTTCTTGCAC | 1888 |
| 1439 | ACCAAGTGTTGCTGGTGCTG | 1889 |
| 1440 | AAGAGTGAAACAGGTGCTCC | 1890 |
| 1441 | CTGGAGGGGCGGGAGGCCCC | 1891 |
| 1442 | AAGAGAAGAACCTGGACCTC | 1892 |
| 1443 | CCTAGAGAGAAAGGTGTGCC | 1893 |
| 1444 | TTGCAAACAACATGTTGGGA | 1894 |
| 1445 | GGTGGCAAGAAGCAGAGAAT | 1895 |
| 1446 | TGTGCTCCATGGTGATGGCC | 1896 |
| 1447 | ATGTCGCAGGGGGCGGCCCC | 1897 |
| 1448 | AGACAGAGCAGCCGTCGTGC | 1898 |
| 1449 | AAGGAAGACATCGGAGTCCC | 1899 |
| 1450 | AAGAGGCCCAGAGGTCTTCC | 1900 |
| 1451 | CCCAGACGACCTGGAGAGCG | 1901 |
| 1452 | CGCTACTCGCAGAGGCCGCC | 1902 |
| 1453 | CCCAGTGAAAAGGGGCCCAG | 1903 |
| 1454 | ATAGGCAACCTGCACTGGTG | 1904 |
| 1455 | TTTTTAGGTTCACGCTGCTG | 1905 |
| 1456 | GCTACTGGTAGAGCTGGTCA | 1906 |
| 1457 | CGTGCAAGACAGGAAGAGGC | 1907 |
| 1458 | CCCGGAAGCCCACAGCACCA | 1908 |
| 1459 | AGTCAGCCCCAAGGGCCCCA | 1909 |
| 1460 | CGCAGCAACCATGGTGGCAT | 1910 |
| 1461 | TCTGCTAGGAGAAGTAGAGG | 1911 |
| 1462 | GTTATTGGTATTCAGTATTC | 1912 |
| 1463 | CTTCAAAGAGCAAGGTTGCC | 1913 |
| 1464 | GCTCAGATGATGGTGTCTGC | 1914 |
| 1465 | CCGCCACCTGGCGGTTGGCG | 1915 |
| 1466 | CAAGCCCAACAGGCAGAGCC | 1916 |
| 1467 | CCAGAAGCTAACGGTCTCAG | 1917 |
| 1468 | GCAGGGCGACCATGGCTCTC | 1918 |
| 1469 | TGGGAGCCATGAGGTGGGGC | 1919 |
| 1470 | CCCCCATGATGGGGACGACT | 1920 |
| 1471 | GTTGAACCCAGTGGACCTCC | 1921 |
| 1472 | GATGATCGCACTGGACATCC | 1922 |
| 1473 | TCCTGAAGACCCTGGCCTGC | 1923 |
| 1474 | GGGCGGCAGCTACGTGCTCT | 1924 |
| 1475 | ACTCAAGATGACTTTGTGCG | 1925 |
| 1476 | ATGCCAGGTGGCATGTTTCC | 1926 |
| 1477 | AACAGATGCTCCTGGATTAA | 1927 |
| 1478 | GCCCCAGATGTCATCCTCCT | 1928 |
| 1479 | CGATGAACGAAATGGAGAAA | 1929 |
| 1480 | GAAAAGAGATGAAGGGCCTA | 1930 |
| 1481 | CTCACCAGACTACGAGACCG | 1931 |
| 1482 | GAACGACATGAAGTACTACC | 1932 |
| 1483 | GCCAGAACCACCTCCTCCGT | 1933 |
| 1484 | CCTGACCCAATTGGCCCAGC | 1934 |
| 1485 | TGCCATGACTGTGGCCTGCC | 1935 |
| 1486 | CGTAGCGATAAGGGAGAGCC | 1936 |
| 1487 | GTTTTAACAGCTCTCCACCC | 1937 |
| 1488 | AAAGAGGACATTGGCCCTCC | 1938 |
| 1489 | ACCGCAGGGAGGCCGCCAGC | 1939 |
| 1490 | TCAGGAACCTCCTGGACCAC | 1940 |
| 1491 | CCCCAGAGGTGTTCACACAC | 1941 |
| 1492 | GTCCTAGCAGGTGCCCCCGT | 1942 |
| 1493 | CCTCTCCACGGCGCGGCCAT | 1943 |
| 1494 | TCTAGAGAAATGGCCAGCGG | 1944 |
| 1495 | AGAACTATTTGTTTAGTTCT | 1945 |
| 1496 | CGCTCATTTAGGGAAGGAGA | 1946 |
| 1497 | GCGGTCAGCCACCTGGCTGG | 1947 |
| 1498 | TGGTTGATTTGTCAGCAATC | 1948 |
| 1499 | GACGACCAGAATGGCGTGCC | 1949 |
| 1500 | AACTAAGCGAATTTGGATAA | 1950 |
| 1501 | TTCCTCGTGATCGCAGGCTT | 1951 |
| 1502 | AAAGATCATGCTGGTCTTGC | 1952 |
| 1503 | CCTAGACAAAGAGGAGAACC | 1953 |
| 1504 | TCCCTAACAGAGCCCGGGGA | 1954 |
| 1505 | AGAACCACAGAGCTAAGCAA | 1955 |
| 1506 | ACATCAGCAAGCCTTTACTT | 1956 |
| 1507 | CCAGACCAACCCTGCTCTGG | 1957 |
| 1508 | GCAACCAGTTGTACCGCGAG | 1958 |
| 1509 | TAATCACTTGGCCATGTAGT | 1959 |
| 1510 | CTTTCACACACAGTAGTCCC | 1960 |
| 1511 | CGTCCAGGTAGTGCGTCTTC | 1961 |
| 1512 | TCTCATCCTCGGGGGCCAAC | 1962 |
| 1513 | CCTCAAGGGCCTGTCTGACC | 1963 |
| 1514 | CCCAGGTTTCCAGGGAGCAA | 1964 |
| 1515 | TGCAAGCACCTGCAAGAATG | 1965 |
| 1516 | CTAGGCAGGACACATCTCAC | 1966 |
| 1517 | CACACAGAGCTCATTGTAGA | 1967 |
| 1518 | GTGATGAAAAACTCTCCCGC | 1968 |
| 1519 | TCCCCCTGCACATGCGGGAC | 1969 |
| 1520 | TTCACTCCAGGTAGGGCAGA | 1970 |
| 1521 | AAGGATGAAACAGGTGCTCC | 1971 |
| 1522 | AAGGTTTAATAGGCAGCTGA | 1972 |
| 1523 | TCTGATCCTAGTGGACTCCC | 1973 |
| 1524 | TAGAGCTTGAGGTTCACATC | 1974 |
| 1525 | ATACATTTTTACCAAGAAGT | 1975 |
| 1526 | TTGGGAAGGTTCTTACATGA | 1976 |
| 1527 | TATCCACTACAACCCGCTGC | 1977 |
| 1528 | CCCGACCCCCCAGGGCCGCC | 1978 |
| 1529 | GCAGTGCTGAGCAGAGCAGC | 1979 |
| 1530 | CATTCTTAAGTGTGAAGGTC | 1980 |
| 1531 | GTGCCGGAAGACGGGGCTGC | 1981 |
| 1532 | AAGGCAGTAGTTTTTAGTAA | 1982 |
| 1533 | TGAAGTTGTCCAGGTGAGCC | 1983 |
| 1534 | CCGATGATACCAGTTTCGAG | 1984 |
| 1535 | TTACGTATATTCATGGAGTA | 1985 |
| 1536 | CCCAGGTTGCCAGGGGCTCC | 1986 |
| 1537 | CATAGGCATCATGTCCATCC | 1987 |
| 1538 | TGAAAGAGCCACATATAAAG | 1988 |
| 1539 | CTCTTCTACAATATGTAGCT | 1989 |
| 1540 | ATTTTATGCATCTGGTGGAA | 1990 |
| 1541 | GCAGCTGTAGTTTAGTCCTA | 1991 |
| 1542 | ATGAAATCTGGCCAGCTTTG | 1992 |
| 1543 | TTGCAAGCAAAATAGTTCTG | 1993 |
| 1544 | CCATTATGTTGAGATTGGGG | 1994 |
| 1545 | CAGAGAGAGCTGGGGCGGAT | 1995 |
| 1546 | GCAGAACCCCCTGGAGGTTC | 1996 |
| 1547 | GCCTAGCCCTCGGCCATCGC | 1997 |
| 1548 | TGTTGAGTACCAGGGAATGA | 1998 |
| 1549 | GCTGCAGCGCATTGCCAGCC | 1999 |
| 1550 | CATCTCAGTAGACTTTTACC | 2000 |
| 1551 | GGAAGCTAGGAGCTGGGGGA | 2001 |
| 1552 | CGACCTCTACATGGTCTCTC | 2002 |
| 1553 | TTGGCCAAGTGCCTGTGCGG | 2003 |
| 1554 | TCCTAGCACTCCAGGTCCTC | 2004 |
| 1555 | GGGCTAGAGAAGGACCTGGA | 2005 |
| 1556 | GTTGAACGTGAGCCGCTTCT | 2006 |
| 1557 | GCTCAGCAGGGCCTGCAATT | 2007 |
| 1558 | ACCAGAAAGCATGGGGAACA | 2008 |
| 1559 | ACAACAGCATGCCACTGGCC | 2009 |
| 1560 | GTATACTGTTGATGTGTATG | 2010 |
| 1561 | TTAATCACAGCTTTTGGTCC | 2011 |
| 1562 | ACTTTAGTCAGTTTCCTCAT | 2012 |
| 1563 | GTCGCAGTAGGGCGCACGCT | 2013 |
| 1564 | GCAAGGGCCCCAGGACTTAG | 2014 |
| 1565 | CTAGGGGGCCATGGATGCTA | 2015 |
| 1566 | CCCAGAGAAAGATGGCCCAA | 2016 |
| 1567 | CAGAGTCTTCCTGGTCTGGC | 2017 |
| 1568 | CACATGATACGACCTGCAGA | 2018 |
| 1569 | GGGCTAGAAGAAGCAGTGCA | 2019 |
| 1570 | GCGGACACGGTACCTGGGCT | 2020 |
| 1571 | CCAGAACCTCCTGGTGCTAT | 2021 |
| 1572 | TCTGCCAGAAGTCCCCGTAG | 2022 |
| 1573 | CCAGAAGAGAAGGGAGAAGC | 2023 |
| 1574 | TGCTGACATTCGAGGCCCTC | 2024 |
| 1575 | TTCGAGGGTATACATGGGCT | 2025 |
| 1576 | CACCAAGGATCTGATTTAGT | 2026 |
| 1577 | AATCTAGGATATATGTTTCT | 2027 |
| 1578 | AAGAGTGAAAACGGTGTTGT | 2028 |
| 1579 | GCAGGGCATGTCTCTGCAAA | 2029 |
| 1580 | TCTCAGGAGATGAAGTCTTC | 2030 |
| 1581 | TTCTCCTAAGAATGGGAATA | 2031 |
| 1582 | CAGCAAGGCCTGCAGGCTGT | 2032 |
| 1583 | GACTGAGATGGTGATCTCGT | 2033 |
| 1584 | GCCAGTGCTAATGGTGCTCC | 2034 |
| 1585 | ACACTAATTTCCGCCAGGTA | 2035 |
| 1586 | CCACAAAGACAATGTGGTAG | 2036 |
| 1587 | CGACAGGGACCAGTCGGTGG | 2037 |
| 1588 | TAAGATGCAGGCTCTAGAGG | 2038 |
| 1589 | GGAAGTGACGTGCTGTGCTG | 2039 |
| 1590 | AAGCGTTAAATATCGGCATC | 2040 |
| 1591 | AAGCTGCAATCTGAAAACAA | 2041 |
| 1592 | CCCTGGTGAGACACCTCCTC | 2042 |
| 1593 | GTGAATGTAGAGGTCTACAG | 2043 |
| 1594 | TTTCAACGCCCCCGCCACCG | 2044 |
| 1595 | ATACTACATCAGGATTTTGC | 2045 |
| 1596 | TCCTAGCATTCCAGGAAAGG | 2046 |
| 1597 | ACCTTAGCACCTTCTTCCAC | 2047 |
| 1598 | GTCACGGGAAACTGATCCTC | 2048 |
| 1599 | GGCCAACCACAGGTCGGAGG | 2049 |
| 1600 | GCGTTCCAAGGAGGACGGCC | 2050 |
| 1601 | CCACCATGTACTCTGCAGGG | 2051 |
| 1602 | CTCAAGGGTAAAATTCGCTG | 2052 |
| 1603 | ATACTACAGGAGAGAGAAGA | 2053 |
| 1604 | CTAATCCCCTAAGGAAACAG | 2054 |
| 1605 | GTTCAAGAAGAAGTCGCTGG | 2055 |
| 1606 | CGGAGTCTTGCAGGACCACC | 2056 |
| 1607 | GTCCGGTATCTAAAAGACTA | 2057 |
| 1608 | AAACAAAGGTGCTTCAATAA | 2058 |
| 1609 | AGTTCTCAGGCTGTGTAATA | 2059 |
| 1610 | TAATTCATTCTAATTGGTTC | 2060 |
| 1611 | GGAGGGTAAGGAGTGCAGGT | 2061 |
| 1612 | CCTAGGGGCGCTCGGCCGCC | 2062 |
| 1613 | CCCAGGGAGAAGGGGAGCAT | 2063 |
| 1614 | GATAGACAGCCTGCACTGGT | 2064 |
| 1615 | CACAAGCACTTTTTCTGATT | 2065 |
| 1616 | TGTTTCAAGTGCTGGCAGTG | 2066 |
| 1617 | GCCCAAGCCTTGCCGGACGC | 2067 |
| 1618 | ATCTCATTCTGCAGGCAGCA | 2068 |
| 1619 | CGGGAGTAGATGATTAGAAA | 2069 |
| 1620 | TGGTGAGACAGGATGTGGCC | 2070 |
| 1621 | GCTAGTCCAGGGGCAGCACC | 2071 |
| 1622 | CACTCTATTTCTGCTGGCTG | 2072 |
| 1623 | CACCCACCAAATTCCAGCTT | 2073 |
| 1624 | GGAGGAAAAAGATGAACCTT | 2074 |
| 1625 | CAGGAGTCTCCCCTGGGGGA | 2075 |
| 1626 | CCTGACCCAGCTGGCCAGCC | 2076 |
| 1627 | CCGTGGAGCAGAGCTCGCAG | 2077 |
| 1628 | AGGAACAGTTCATTGATAGC | 2078 |
| 1629 | TCTTACAGAATGTAGCCTTT | 2079 |
| 1630 | GTCTCAAACATTGTCCTGAA | 2080 |
| 1631 | ACTAAGGACTGGCAGGCACT | 2081 |
| 1632 | CTTCAGGCTGCCCCGGCTGC | 2082 |
| 1633 | GCCTGACCATCAGGAACATG | 2083 |
| 1634 | TCACACTGACCCAGACCTGG | 2084 |
| 1635 | CAGAAGTGAAAGAGGATCTG | 2085 |
| 1636 | GGCACTGTGACATCGATGAG | 2086 |
| 1637 | CCAGAATTGGATGGCATCCC | 2087 |
| 1638 | GATCGAAAACGCGGCGGCGG | 2088 |
| 1639 | AAACGTCCACGCGGTGCGGG | 2089 |
| 1640 | AGCACACACCTTGTCCAGGT | 2090 |
| 1641 | CCTTAGGGGCTTTGCCCCTG | 2091 |
| 1642 | GAATGAGCTTCTTGCAGCAA | 2092 |
| 1643 | CTGGCAGTAGTAGGGGCTGA | 2093 |
| 1644 | GGAGTCATGAGGTACCTGCA | 2094 |
| 1645 | ATGAGCACGATGTAGCTATA | 2095 |
| 1646 | TTCCACTTCACCGGCACCTG | 2096 |
| 1647 | GGCACACATCGAGGAGCTGG | 2097 |
| 1648 | ACACCAAGCCGGCTGGCTGC | 2098 |
| 1649 | CTCAGGCCACACTGATTCGC | 2099 |
| 1650 | TCTCTAATAAGCAGTACTGT | 2100 |
| 1651 | TGGTTTGAACTGGATATCGG | 2101 |
| 1652 | CCCAGTCAAGATGGTATTCC | 2102 |
| 1653 | TCCACTATACTCTCAAGGAT | 2103 |
| 1654 | AAGTCACAGACGCTTCTTTT | 2104 |
| 1655 | AGCACAGCATCGTCACCAAC | 2105 |
| 1656 | ATGTGAAGATTGCCACCTAC | 2106 |
| 1657 | TGCATGAGCCGCTCTAGCAT | 2107 |
| 1658 | GAAACAGGGTTTCACCATGT | 2108 |
| 1659 | GAAGAAAAGAGAGGCCCTAA | 2109 |
| 1660 | TAGTGCAGCAAAAGCGCGCC | 2110 |
| 1661 | CAAAGAAGAGTGCGGCAGCG | 2111 |
| 1662 | AGAAACATAGGCACATCCAC | 2112 |
| 1663 | TCTCGCCCAACCCCAACCTC | 2113 |
| 1664 | TTTGATTGGTCCTGTTGGCT | 2114 |
| 1665 | TTCTCATGTGGAATTTTCCA | 2115 |
| 1666 | CCTCCAGGAGGGGTAGGGGT | 2116 |
| 1667 | GAGTACCAGAAGAGATACAG | 2117 |
| 1668 | CGATGAGGTACTTCAGGGTG | 2118 |
| 1669 | ATTGAACCACCAGGGCCTCG | 2119 |
| 1670 | CGAGTAGCAAGAGGTGGAGA | 2120 |
| 1671 | CTGAGTCAGCATCTGCCAGC | 2121 |
| 1672 | TCCAGATAGGTGTGCTTTGT | 2122 |
| 1673 | TGATGACACCAAGGGAGAAA | 2123 |
| 1674 | AGCACTGACGTCTGGGGGAG | 2124 |
| 1675 | CTGAGATTGAGAAACCTGGG | 2125 |
| 1676 | AAGCTTTCAACATCTGTGAA | 2126 |
| 1677 | GGAGCAAGGTCCTGCTCCGA | 2127 |
| 1678 | CTATCTTTTCCTCTAGAGTC | 2128 |
| 1679 | AAGTCATTGCTGTATGAGCC | 2129 |
| 1680 | AGTCCAGCTAAAGCCCTGGC | 2130 |
| 1681 | CCCAAGTTTTCCTGGCCTCC | 2131 |
| 1682 | CATGACCATCCAGAACGCCC | 2132 |
| 1683 | CCTAGAGAACAAGGACCCCC | 2133 |
| 1684 | TTTGGGTCAACTGTCCACCT | 2134 |
| 1685 | GGCCTACATGTGTCCTGCCT | 2135 |
| 1686 | ATACCACCGGGCGGCAAAGC | 2136 |
| 1687 | AGATGAGAAAGCTTATCTAA | 2137 |
| 1688 | CGAGACAAGCCGGGGCTCCC | 2138 |
| 1689 | ACCCTGCTATGCCAGCTGGG | 2139 |
| 1690 | TCCAGGTTAGGCCACTTCAC | 2140 |
| 1691 | CCTGATGCTATAGGTCCATC | 2141 |
| 1692 | CATCCAGCTCATAACCCTAA | 2142 |
| 1693 | CTTCAAGGAATGTGAGGTAT | 2143 |
| 1694 | AGGACCAGAGGGACCTGGGA | 2144 |
| 1695 | TATTGTCACAGATACTCCAG | 2145 |
| 1696 | GGGCCTAGGTTCGGAATGCC | 2146 |
| 1697 | TCTACAGGGACTTCCGGCAG | 2147 |
| 1698 | TAGCCATGACCTAATTATTT | 2148 |
| 1699 | GCTCAAAGCTGACCCACCGT | 2149 |
| 1700 | ACAACATACAAAGTGGGGAT | 2150 |
| 1701 | CACAACACTTTTGGGGGTGA | 2151 |
| 1702 | ACAGAACCCCCTGGTCCACA | 2152 |
| 1703 | CAGCCATGCCTTGTCGCAGA | 2153 |
| 1704 | GAGAGTCAGAAGAGATGCAC | 2154 |
| 1705 | TTCAATATGTTGATGGAGAG | 2155 |
| 1706 | AAAATAAAGTAGGAGTACTC | 2156 |
| 1707 | TGCAGCAGGAGGGAGGTAGG | 2157 |
| 1708 | CCAGAAGAGAAGGGATCGCC | 2158 |
| 1709 | CGCGACTACTGCGCGCGGGA | 2159 |
| 1710 | CATCAACACCAACGTGCAGG | 2160 |
| 1711 | TTGTTAAGGGCTATGCCGGG | 2161 |
| 1712 | GATGGAGACACTGTACCTGC | 2162 |
| 1713 | GGGGCACAGGTAGAGCTGGG | 2163 |
| 1714 | CAGAGATGAAAGAGGATCTG | 2164 |
| 1715 | AAATGACATCCCAGGAGAAA | 2165 |
| 1716 | GCTAGCCCCAATGGATTTGC | 2166 |
| 1717 | CTACCTTGCCCATGAAATCT | 2167 |
| 1718 | AACAATACAGCTTTTAGAAA | 2168 |
| 1719 | CAGCTGTCAGGCTTTGGAGC | 2169 |
| 1720 | ATCTCAGCCAGGGGGGCCTG | 2170 |
| 1721 | GGAGCGACCAGGAGGCCATC | 2171 |
| 1722 | GGCGCCGTAGGCCACGGCCC | 2172 |
| 1723 | TCTAGTCCAGGACCCGGTGC | 2173 |
| 1724 | CAGGCCACAGCCAGGGGAGG | 2174 |
| 1725 | GGAGACGTAGAGGGACAGCA | 2175 |
| 1726 | GACAATGATGTTAGATGCAG | 2176 |
| 1727 | GTTTTAGATAGACCTTAATG | 2177 |
| 1728 | CCCAGCGTTGCTGGGGCTCC | 2178 |
| 1729 | GCCGAGCAGGCTGGCCACCA | 2179 |
| 1730 | CATGAAGTCATGTCCCCGGA | 2180 |
| 1731 | AGATAAGCAGTCCCTCCAAA | 2181 |
| 1732 | GTTGATAACGCTGGTCCTGC | 2182 |
| 1733 | CAGCCACACCAGTACTTCAT | 2183 |
| 1734 | CCACAGGCACCAGGTGGGCC | 2184 |
| 1735 | GGAACCACCAGACGTTGGCC | 2185 |
| 1736 | CCTGGCAGTAGGCACCCAGC | 2186 |
| 1737 | TACTTACAAGCTAAGGATCA | 2187 |
| 1738 | AAGTTCTTAAAGTTCCTGGT | 2188 |
| 1739 | AAGATAGAAAATTAATTATT | 2189 |
| 1740 | AAAGATGACAAAGGAAATCC | 2190 |
| 1741 | TGGTTAAACTGCTCTGATCA | 2191 |
| 1742 | CTCTCAGAATTGGTCAAAGA | 2192 |
| 1743 | GAGCAAATTTGGATAAAGGA | 2193 |
| 1744 | GAAGAAAAGAACTGTGAATT | 2194 |
| 1745 | CCTCTCAGTATTCTTGGACC | 2195 |
| 1746 | CAGCAGAAGAAAAGGTGAGA | 2196 |
| 1747 | GCTGATGAGAAGGGTCCCTC | 2197 |
| 1748 | CTAGGGCCGGCAGCAGTGAC | 2198 |
| 1749 | ATGGGCTAAGCGCTCAGTTT | 2199 |
| 1750 | GGTCAGGGTGGCTTATGCAA | 2200 |
| 1751 | TCTGAGTTTCGAGTCAGGGG | 2201 |
| 1752 | GCACTAAGAGCACTGCGAAC | 2202 |
| 1753 | CATGGACCCATGTGCTGGTG | 2203 |
| 1754 | CGGAGGCGGCCCACGATGGA | 2204 |
| 1755 | TGGCCGTCACATTGTTGTCC | 2205 |
| 1756 | GGTCCAGACCCACTGGCTGC | 2206 |
| 1757 | CCTCCAGGATTCCAGAACCT | 2207 |
| 1758 | GAAGGGCACATGCCAGACAC | 2208 |
| 1759 | ACCTGATGTGGTTGGTGCTG | 2209 |
| 1760 | TTGCTAAGGGGTATCTACAG | 2210 |
| 1761 | CCCTGATTCAAGCGCACCCT | 2211 |
| 1762 | GCTAGTCCTCAGACTTCACG | 2212 |
| 1763 | GAACATGGTCGCTCTGGACA | 2213 |
| 1764 | GGATCAAGCCTTCACGTTGC | 2214 |
| 1765 | CTGGGACACTTCTTCTTCTC | 2215 |
| 1766 | GCTGCAGCCGGGAGAGTTCG | 2216 |
| 1767 | GCGCGAGCAGCGCAATGGTC | 2217 |
| 1768 | AATCAGTTGAAGCGCCATTC | 2218 |
| 1769 | CTACCCGTCCGTGAACTTCC | 2219 |
| 1770 | CTGAGGGGCCATGGATGCTA | 2220 |
| 1771 | CCGGATGAGAAAGGTGAAGG | 2221 |
| 1772 | TGGGGACCCAGCGCACGCAG | 2222 |
| 1773 | GCCATGCGCGCCTGGAGAGC | 2223 |
| 1774 | AAGCTCACTCCATGCAGTAC | 2224 |
| 1775 | CTGTGGCAGAGGGACAGGAC | 2225 |
| 1776 | GAGCCTCAGGTGGCAGCCCC | 2226 |
| 1777 | CAGGCTAGCCTGGTGGGCCC | 2227 |
| 1778 | CTCGAGGCACTCTGGCAGCA | 2228 |
| 1779 | CCGCCCACACCTGCAGGGAG | 2229 |
| 1780 | CAGTATGTGCAGGTACCCTG | 2230 |
| 1781 | GCATGGTGAACACGTCCTGC | 2231 |
| 1782 | GGACATGAGTTTCAGCACGC | 2232 |
| 1783 | AGAGATGAAACTGGCCCTCC | 2233 |
| 1784 | GGCTTAGACCTGGGAGGCGG | 2234 |
| 1785 | AAGCTACACTCCAGCTGGAT | 2235 |
| 1786 | CCTGACCCTGTTGGTGCTGC | 2236 |
| 1787 | CCTGTCGATGTAAACCACGA | 2237 |
| 1788 | TCGCTATTCAATTTCCTGTT | 2238 |
| 1789 | AATAGTGCCCCTGGACAAAG | 2239 |
| 1790 | CCTAGGAGCCCGGGAATACC | 2240 |
| 1791 | ATTTGCAGTGGACGATGGAA | 2241 |
| 1792 | GCGGCTAGGGCTTGGTCTGG | 2242 |
| 1793 | CTGGGCAGGGATGGCTGCCT | 2243 |
| 1794 | TCTTCACAGGGAAGTTTTGG | 2244 |
| 1795 | TCCAGCCTAGGCCTTGAACC | 2245 |
| 1796 | ATCCTACAGGTGCAGCACCA | 2246 |
| 1797 | CACCTGAGAAGTCGGCTGAG | 2247 |
| 1798 | TGCGGCAGAAGGCTGATAGG | 2248 |
| 1799 | CCCGATCCCCCTGGTACATC | 2249 |
| 1800 | CCGTGACAGTGATGGAAGTG | 2250 |
| 1801 | CCCAGTCCTGCTGGAAGTCG | 2251 |
| 1802 | AATGCGCCACAAAACCCTGC | 2252 |
| 1803 | CACTCAATCCCAGGGGCTCT | 2253 |
| 1804 | TGTACGAAAAATGTGAGTTA | 2254 |
| 1805 | CGCTATCCAGCAATACCTTG | 2255 |
| 1806 | AGTGATGAAGAAGGAAAGAG | 2256 |
| 1807 | CCCTAGTGGGACACCTCCTC | 2257 |
| 1808 | GATCCTGATGTCGGAGGACC | 2258 |
| 1809 | CCACTACAAACTTGTTGGTG | 2259 |
| 1810 | GTATTACCGCCCCCGGTAGT | 2260 |
| 1811 | GACTATGTGCATTTTAGGCC | 2261 |
| 1812 | GGCCGCAGCAAGTGTGAATG | 2262 |
| 1813 | CCCTGAGGAGCCTGGTCTCA | 2263 |
| 1814 | TGTCTATCCTAGGTGTTTGT | 2264 |
| 1815 | AAAGATGATGCTGGCCAACC | 2265 |
| 1816 | GTAAGAGTCCACACCAGCTG | 2266 |
| 1817 | GCTGATCCTGCTGGTCCCGC | 2267 |
| 1818 | GTGGGTATGAACCACTGTAT | 2268 |
| 1819 | CAAAGCATTCGGTGATGAAG | 2269 |
| 1820 | ACCTCAGCTAGCGAGCTCTC | 2270 |
| 1821 | ATGGGAACCGCTACTTCAAG | 2271 |
| 1822 | GCAGCACGACACTGTGGCCA | 2272 |
| 1823 | GAGTCATCCCCTCTTGTTCA | 2273 |
| 1824 | CGATGTCTACTGCCCTCTGG | 2274 |
| 1825 | GACCTGCACCACACCTTCAT | 2275 |
| 1826 | CACCCACTACCTGGTGTTAG | 2276 |
| 1827 | CGCCCATGTGCACTCGGATG | 2277 |
| 1828 | CCCTAGCTTCGCTGGTGAGA | 2278 |
| 1829 | GGTGGCCCATGTAGCCTGGG | 2279 |
| 1830 | CCAGCAAGGCGGCAAAGAGC | 2280 |
| 1831 | CCACTTTCTCATGTGCAACC | 2281 |
| 1832 | TGGGCCACTTGACGCGGTCC | 2282 |
| 1833 | GGAACGAAGCCGCCCAGGAA | 2283 |
| 1834 | TCGAGTTCCTGGAAAGATAA | 2284 |
| 1835 | AGCTCTAGTCCATGATGAGG | 2285 |
| 1836 | GCCATTCGCTAAACCCCAAC | 2286 |
| 1837 | AGCCCGCCACTGAGGAGGCC | 2287 |
| 1838 | AGTAGGAAACCAGGAGCTAA | 2288 |
| 1839 | TTGGTCCTATCGGTTCATGA | 2289 |
| 1840 | CGAGCAGGTGCACAAGGTCA | 2290 |
| 1841 | GTGTAATGGAGGGCCAGGGG | 2291 |
| 1842 | CAGCTAGTCTCCTGAGAAGA | 2292 |
| 1843 | CAGCCCCTACTTCCTGCATA | 2293 |
| 1844 | GGGGACTGGTTTGCCATCCG | 2294 |
| 1845 | AATGACTCTCCTGGTGCCCC | 2295 |
| 1846 | TTGGCAGATGGCACCAGTGC | 2296 |
| 1847 | CCTGAACCCCGCGGTATTCC | 2297 |
| 1848 | TACTGCTAGGGTGGGCGGAC | 2298 |
| 1849 | ACGAGTTTCTCTGGGGGCAC | 2299 |
| 1850 | GCCTGCTAGGCCACTTCACG | 2300 |
| 1851 | GTGTGAGAACGACCTCTCTC | 2301 |
| 1852 | GCTCTACTCGCCGCCGAGGT | 2302 |
| 1853 | CCTGATCCTGCTGGAGCCAC | 2303 |
| 1854 | GCTACTTGCGCTTCTCGTGC | 2304 |
| 1855 | GTCGGACCAAGTGGCGCAAG | 2305 |
| 1856 | CTCAGAAGTCAGATGCACCA | 2306 |
| 1857 | CCTCATTCTCCATTCTTACC | 2307 |
| 1858 | GGATGCTGGTGAAGCCACCT | 2308 |
| 1859 | CTACTTCTCAGTCAAGAGCT | 2309 |
| 1860 | AAAGATCCAGCTGGGATACC | 2310 |
| 1861 | TCCTGAGTTGCTGTCCCCCA | 2311 |
| 1862 | CCTAGAGTCAACGGTGCTCC | 2312 |
| 1863 | GTTAGTCCCCCTGGCTTCGC | 2313 |
| 1864 | AGTGTGAGCCGCCATCGGCG | 2314 |
| 1865 | CCACTAGAAACTTTCCCCCA | 2315 |
| 1866 | TGCGCAGGCGCCGCTGCTGC | 2316 |
| 1867 | TCTGCAAACACCTTTTCTAT | 2317 |
| 1868 | GGTTTAAATGGTTTCCCCAG | 2318 |
| 1869 | CCCCAGAGACGATGTAAGTG | 2319 |
| 1870 | CAGGATCCCCCTGGTCCTCC | 2320 |
| 1871 | TCCCCACTCCATTGTGGCCC | 2321 |
| 1872 | TGGCGTATGGAAAAAGCAAC | 2322 |
| 1873 | CTCAGCTGGAGAGAAGTCGA | 2323 |
| 1874 | CACCTAGCTCTTGAATGACA | 2324 |
| 1875 | CGTAGTCTTCCTGGAGCTGA | 2325 |
| 1876 | GGACGTCCCGCCGGGGTCGC | 2326 |
| 1877 | CAGGGCTACAGCAGCAGCAT | 2327 |
| 1878 | GCCTCCCAGCAGGTGCGATG | 2328 |
| 1879 | TCTTACTCGAGATGTGATGA | 2329 |
| 1880 | GATGACCCTCCTGGACTCCC | 2330 |
| 1881 | ACTGGCCTAGAGCGGCCAGG | 2331 |
| 1882 | ACCTCAAAGGGAGAAGCCAA | 2332 |
| 1883 | TCAGGCTAAGGGGACAGATG | 2333 |
| 1884 | CAGCACCTCCCAGTTCTGAG | 2334 |
| 1885 | GCTGAGGCTCCCGGCCTCCC | 2335 |
| 1886 | GACTCTTATGCAGGTTCGGG | 2336 |
| 1887 | CCTATGCAGCCAGCACACCT | 2337 |
| 1888 | GGATCAGCTTCATTGTGCTG | 2338 |
| 1889 | TCTTAAGGGCTGGATGGTGG | 2339 |
| 1890 | CATCTATGTGGCCTGTCTCC | 2340 |
| 1891 | CCATCAGCCTGGGCAACGTC | 2341 |
| 1892 | AGTCCAGCCAGAAGGCGTGC | 2342 |
| 1893 | AAGGATGATGCCGGTGCACC | 2343 |
| 1894 | GCTAGGGGGAACTGAGCACC | 2344 |
| 1895 | TCCTTTATGGAGTTTTAAAT | 2345 |
| 1896 | TCCAACTACAGCGTCTCCTT | 2346 |
| 1897 | ATGCCCTAGGGCCCCTGGGG | 2347 |
| 1898 | GGACTATGAAATAATGCTGT | 2348 |
| 1899 | GGGCCTAGTCTTCCAGGGTG | 2349 |
| 1900 | GGCTACGCAGGGGCCCCCGT | 2350 |
| 1901 | ACTGATTTCCCTGGTGCTGC | 2351 |
| 1902 | CCCCAATGACGGCCAGCAGG | 2352 |
| 1903 | ACTGGAGCCCCGGAACTCAA | 2353 |
| 1904 | GCATCATCCCATGTCTTTAG | 2354 |
| 1905 | CGTGAGCTTCCTGGTGAGAG | 2355 |
| 1906 | GCTCTCAGAGCACCGGGTGC | 2356 |
| 1907 | CATGCACTCCAGGTGGGCGC | 2357 |
| 1908 | ACCTGACCCACCTGGACATT | 2358 |
| 1909 | CCAGAGCCTCGAGGTAACAG | 2359 |
| 1910 | CTTGACCTTGAAGGTCATGT | 2360 |
| 1911 | CCTGATGCTGCTGGTACTCC | 2361 |
| 1912 | TGACCACCTGGCCTCCTACC | 2362 |
| 1913 | TGTGGCTGCAGATGGTGTGT | 2363 |
| 1914 | GGGGGGCAAGCAGCTGCTGC | 2364 |
| 1915 | AATCTAGGTCTGGTCTCCAT | 2365 |
| 1916 | ACTACCTATTATCTGAGCTC | 2366 |
| 1917 | CCGAGAGCCCCAGGTCGAGA | 2367 |
| 1918 | CTTCCAGAGGTGCTTGAATC | 2368 |
| 1919 | GTGGATCCTGCTGGCAAACA | 2369 |
| 1920 | CCGGGCAGATGGTCTCAGTG | 2370 |
| 1921 | CCAGGAGCCTGTGGGGCTCC | 2371 |
| 1922 | TCCTAGTCCAAAGGGTGACA | 2372 |
| 1923 | GCTGCAGGTGACCGAGGGCT | 2373 |
| 1924 | GGAGACCCTCCTGGAGTTGC | 2374 |
| 1925 | GCCGATGCACCTGGAGCTCC | 2375 |
| 1926 | CATGGATGCTCAGCGTGATG | 2376 |
| 1927 | TGGGGGATCACTTCCGCATG | 2377 |
| 1928 | GATCCGGACAGGGGCCTCCC | 2378 |
| 1929 | CACTCAGGAGAAAGGGTCGG | 2379 |
| 1930 | TCTCCCTCTACACACTGCCT | 2380 |
| 1931 | CCCAGGCTGGCAGGACACAA | 2381 |
| 1932 | AGATTAGATGAACCGCATGG | 2382 |
| 1933 | CCCAAGTGCCCCTGGCCCCA | 2383 |
| 1934 | GGGCCCTTAGTGCGTCATCC | 2384 |
| 1935 | GCCAGCCCTCCAGGACCTCC | 2385 |
| 1936 | TGCTACTGCTGTTGTTGCTG | 2386 |
| 1937 | CTTACTCGCCGGAGTCCCCT | 2387 |
| 1938 | CTTCGCTTCATCCTCTCCTC | 2388 |
| 1939 | GTCCTACTGGTCCAGGGGGC | 2389 |
| 1940 | TCCGGTAGAACAGGGACTCC | 2390 |
| 1941 | CCTGAACCCCAGGGTCTTCC | 2391 |
| 1942 | CGTCGTGCTACAAGCCAACG | 2392 |
| 1943 | ATTGCTGGATGACTGGATGG | 2393 |
| 1944 | CAAGTACTCGTGCTAGAACT | 2394 |
| 1945 | ACCACCTGGTACTTGCTGGC | 2395 |
| 1946 | GGCCCAGCCTGCCAAGAGGA | 2396 |
| 1947 | GAGTTACTGCATTGCTGACC | 2397 |
| 1948 | CGTCTCAGTCATGGTACCTG | 2398 |
| 1949 | AGCTCGTACCCAACCAGGTT | 2399 |
| 1950 | CTTCAGGGAGCGCTTTTCTA | 2400 |
| 1951 | CCACCATGAGGCTAGGAGGA | 2401 |
| 1952 | TCACTCCCCATTCACCCTGA | 2402 |
| 1953 | CGTGATCCACCAGGCTCAAG | 2403 |
| 1954 | CCACATGTCCATCATCGTGC | 2404 |
| 1955 | GGCCCCCACCGCGGGTAGGT | 2405 |
| 1956 | CCAGAGCCCCCCGGCCCCAA | 2406 |
| 1957 | GACAGTGCTCGAGGCAGTGA | 2407 |
| 1958 | GGGCTAGAGACCCCCAAGGC | 2408 |
| 1959 | GCACTCAGGGCGCCTCTGCC | 2409 |
| 1960 | TCTAGCCCTGCGTGGAGCCC | 2410 |
| 1961 | AGCTCTATCGCTGGGCAAAG | 2411 |
| 1962 | CCAGAGCAACCAGGCCCTCC | 2412 |
| 1963 | TCAGCCTAGTTCCAGGGGAT | 2413 |
| 1964 | TGTACCCGTAAGGGGAAGTA | 2414 |
| 1965 | TCTGAGGAGGCTGTAGGGTT | 2415 |
| 1966 | TCGCTCTAGAGGTCGTGGAA | 2416 |
| 1967 | GTAAGTGGCCTCTTTATATG | 2417 |
| 1968 | CACTACGAAAAGGCCTGTGG | 2418 |
| 1969 | CGCTGGCAAGTATGGTGCGG | 2419 |
| 1970 | ACTTTACTGTTCGGAACTAC | 2420 |
| 1971 | CACTACTGCATCATAGATGA | 2421 |
| 1972 | CATCCAGAGTGGGCCTTGCC | 2422 |
| 1973 | AGGATAGTAAACTTATCATC | 2423 |
| 1974 | AGAGCTAGTCACGGTGGAAG | 2424 |
| 1975 | CCTAGGCGTCCTCACGACTT | 2425 |
| 1976 | ACCAGGCTGCGTGAGTGTGA | 2426 |
| 1977 | GTGTTCAATGCAGATGAAAT | 2427 |
| 1978 | GCTCACTGGTATACCAAACT | 2428 |
| 1979 | CTCATGGTGCTCTGGACCGA | 2429 |
| 1980 | CCTAGACCTCCAGGTGTAAG | 2430 |
| 1981 | CCCACTGCAGGATGTGGTGC | 2431 |
| 1982 | CGGCATTCAGCTGACTCGCA | 2432 |
| 1983 | TTTGCTAAGACTGATTACTT | 2433 |
| 1984 | TCATTTAGAACAGTCTACAA | 2434 |
| 1985 | GTCCCTCCACCACAGTGGAG | 2435 |
| 1986 | GCAGCAGGGTCTCGGAGATC | 2436 |
| 1987 | GTGGCACATGCAGCACAGGA | 2437 |
| 1988 | GGGCTCTAGCCCACGTACGG | 2438 |
| 1989 | AGAACTTGCCAAGTGCCCGC | 2439 |
| 1990 | CCTGAGTTCCGAGGACCTGC | 2440 |
| 1991 | CCTCCAGAAATGCTGGTAGC | 2441 |
| 1992 | TTAATTCACAATTCTTGATG | 2442 |
| 1993 | CTGCCAGGGCGATGGGGGCA | 2443 |
| 1994 | CCTTAGCGGCCAGTGGGTCC | 2444 |
| 1995 | GGTAGGCTCTGGCCCACGTA | 2445 |
| 1996 | TGCACTTGAACCAGGGGTGC | 2446 |
| 1997 | CGCCTCGAGCCTGCTCATGG | 2447 |
| 1998 | CTCCATCTCGCAGTAGTCGC | 2448 |
| 1999 | CTGGTAGACAGGCCTGGCAG | 2449 |
| 2000 | GGGGGATGCCCACGGCACGC | 2450 |
| 2001 | AGTGCTGCACTGCCTGGGTC | 2451 |
| 2002 | GGCCATGCATGTGTTCAGAA | 2452 |
| 2003 | GGTGGACTCATCCTGGGGGG | 2453 |
| 2004 | TCAAAGTGCTCCTGGCTCCG | 2454 |
| 2005 | TGTCTCAGTTCTCACTGGTC | 2455 |
| 2006 | AGCTGACTGGCTACACAGCC | 2456 |
| 2007 | CCGCCACCAGGCGCAGGTCG | 2457 |
| 2008 | GGTGGTCCAGGAGCGAGCAG | 2458 |
| 2009 | GTATGATGTGGTGGTGGCAG | 2459 |
| 2010 | AGCACTCCTCAGCATCTGCC | 2460 |
| 2011 | ATCTAGGAACCTGATGACGC | 2461 |
| 2012 | CTTGGATGGGGTCCACACCG | 2462 |
| 2013 | ACTTGCTACTGCTGTTGTCC | 2463 |
| 2014 | AGCCACTGGGTCATGGTCTC | 2464 |
| 2015 | CCCAATGCTGCCTGCATTCG | 2465 |
| 2016 | ACACCCATCGCATTGGAGAA | 2466 |
| 2017 | GTTAGTGCTGCCGGTGCTAC | 2467 |
| 2018 | GGGAGGCTAGAAGCCGCGCG | 2468 |
| 2019 | TGTTCAAGGTGAACCATTAA | 2469 |
| 2020 | CTGCAGGCCTCGTCTGGGGG | 2470 |
| 2021 | TGGCTAGGACTGAGAGACCA | 2471 |
| 2022 | TCCTGCTCAGTCCGCCTTCC | 2472 |
| 2023 | TCCCTATGGGGGGCCACTGG | 2473 |
| 2024 | GCGGCTGCTACCCTAGACGC | 2474 |
| 2025 | AGCCTACCATTCATGGAATT | 2475 |
| 2026 | ACATATCCTTGGCCCTGAAT | 2476 |
| 2027 | CCCAGCCTCCCTGGACCCCG | 2477 |
| 2028 | CGTGGACCGTGGTACCTGGG | 2478 |
| 2029 | CAGCTACAGCCACCTTAGTT | 2479 |
| 2030 | CGATGAGACCCAGACAAGGC | 2480 |
| 2031 | CTAGTTCTCGAGGCCCGCTG | 2481 |
| 2032 | AGCTGCTATAAAGAGCCCAT | 2482 |
| 2033 | TCTCTTAGCGCACAAAGCCC | 2483 |
| 2034 | CTGGAGCCGGCAGCAGTGAC | 2484 |
| 2035 | AGTGAACCTCCTGGCAAAGA | 2485 |
| 2036 | CCCAGACGTGCCTGGACCCA | 2486 |
| 2037 | CCTGTTAGCTCGGAATGGCT | 2487 |
| 2038 | ACCGAGCTGCGTGAGTGTGA | 2488 |
| 2039 | AGGCCCTCAAGGCGAGCTGC | 2489 |
| 2040 | TTGGTAGCAACAGACCCGTC | 2490 |
| 2041 | GTCTATGTGACAGATGCCTC | 2491 |
| 2042 | ATGTCTCACGGTACCTTGTC | 2492 |
| 2043 | GGCTCTACCGGACTGCATCC | 2493 |
| 2044 | TGTATGGACCTCTTTGCCCC | 2494 |
| 2045 | CTTCAGGGAGGTGAACCAGC | 2495 |
| 2046 | CCCCAGACCCCCTGGCCCCA | 2496 |
| 2047 | GACGCAGATCTGGACACCAG | 2497 |
| 2048 | AGCCTACCTCTGGGCCTCCT | 2498 |
| 2049 | GCTAGTCCTCCTGGGCCACC | 2499 |
| 2050 | CCTAGCGCCAGCAGATCCAA | 2500 |
| 2051 | CGTGAAGCTGCTGGCATCAA | 2501 |
| 2052 | CCGGCAAGCCAACACCTTCT | 2502 |
| 2053 | GCCAGGTCAGCGAAGGTCAG | 2503 |
| 2054 | CCTGCTACACCAGGGCCTGG | 2504 |
| 2055 | GCTCCTCAGGGGCGCTCCCC | 2505 |
| 2056 | GCAACCACTGCCTGAAGGAA | 2506 |
| 2057 | CCTCAAGGCCCTGGGGGACC | 2507 |
| 2058 | CTCCTAGAACCCACCCAAAA | 2508 |
| 2059 | TGGCTACAGCTGCAGAGAGC | 2509 |
| 2060 | GGGCTACGCGCACGACTTGA | 2510 |
| 2061 | CAAGGTCTATGTCTTTTCCT | 2511 |
| 2062 | CCTAGTCCAGCTGGCTCCAA | 2512 |
| 2063 | CTGGGAGAATGGCCACCACT | 2513 |
| 2064 | AGAGAAGCTGCTGGAGAACC | 2514 |
| 2065 | CCTGACGCTCCTGGTCATCC | 2515 |
| 2066 | AATGCCACTGTGACCGCAAG | 2516 |
| 2067 | GCTAGCCCACCTTGCTGCTC | 2517 |
| 2068 | GGCCGTGTAGAATGCCCAGG | 2518 |
| 2069 | CGTAGTGCAAGTGGCCCTGC | 2519 |
| 2070 | CTCCTGATGCATTGAGGCAC | 2520 |
| 2071 | CTCGTGCTACAGCCCCCCTG | 2521 |
| 2072 | TCTAGGGAGCTATGCCAGGA | 2522 |
| 2073 | CCGGCTGCAGGATAGGCAGG | 2523 |
| 2074 | TTCACCTAGTCCAGATGCCC | 2524 |
| 2075 | CTCAGTTCCGGATCAGGATC | 2525 |
| 2076 | TGCTCACTGATAGAAAGCTT | 2526 |
| 2077 | GCCAGGGAGCCTGGAAAGCC | 2527 |
| 2078 | TTTCCCCAGATTCCCTGGAC | 2528 |
| 2079 | TCCACTTCACCCCCGGCTTG | 2529 |
| 2080 | GTAGATGCCCCTGGTCCTGC | 2530 |
| 2081 | TCCTCAGTGAGATCATTGAA | 2531 |
| 2082 | TACCTATGCAGAACCCAAAG | 2532 |
| 2083 | AGAGGGGGTAGCACATGGGC | 2533 |
| 2084 | TTTGCTACTGACACATAAAA | 2534 |
| 2085 | CCAGACTTCCCAGGTGCCCC | 2535 |
| 2086 | CACCTAGCTCGTCTCCGTCT | 2536 |
| 2087 | GCTAGTTGGAGCTGACGCTT | 2537 |
| 2088 | ACCTGACCGTGAACCCACAG | 2538 |
| 2089 | CCAAGGCCTCCCGGTCTCCC | 2539 |
| 2090 | CCGGCAGGTCTACATTGAGC | 2540 |
| 2091 | CGGGGTATGGTGGAGTACTT | 2541 |
| 2092 | GGTCTGATCTCTGATCCAGC | 2542 |
| 2093 | AATGAATTGCAAGGTCTGCC | 2543 |
| 2094 | TACTAGGGAGGCTCTGGGCC | 2544 |
| 2095 | ACCCTACTGTGCCAGCTGGG | 2545 |
| 2096 | CGCCAGTCCCCGTGGTGAAG | 2546 |
| 2097 | TCCCACGGGCGCCCGTGCGC | 2547 |
| 2098 | CAAGTAGCTGGCCAACTTCT | 2548 |
| 2099 | GGCTTACGCTTCCAGAGCTT | 2549 |
| 2100 | GAACTACCTCTTGAGTCTTT | 2550 |
| 2101 | CACTACAGGCCCGGCACGTC | 2551 |
| 2102 | TCCCCAGCCGCCTGCAAGGA | 2552 |
| 2103 | AGAAGTGAACGTGGTCTACC | 2553 |
| 2104 | TTTGGAGAGCCACTCCAAGA | 2554 |
| 2105 | CCTACTCCCGGATGTTCTTG | 2555 |
| 2106 | GCCCCACTCACACCTGAGAG | 2556 |
| 2107 | GCTGATCCCCCTGGTCCCGA | 2557 |
| 2108 | GTCAGGCCAGTGCTCGCCAC | 2558 |
| 2109 | TGGACAGCTGGTGGGTCATC | 2559 |
| 2110 | GACTAGGCTCCCAGGGCACA | 2560 |
| 2111 | CGTAGTGAGGTCGGTCCTGC | 2561 |
| 2112 | ATCATGGGCCGCGTGTGGAT | 2562 |
| 2113 | GGGGCCCCATGAGGGAGACA | 2563 |
| 2114 | GCCCAGCCTCCCGGGTCCCC | 2564 |
| 2115 | TCCTAGGGAAGCTGACAAAG | 2565 |
| 2116 | GTATCGGGGGCGGCCCACGA | 2566 |
| 2117 | CCTAGGCAATGATGGCAATG | 2567 |
| 2118 | GGCATGGCTGCTTGGTCTCC | 2568 |
| 2119 | GTGTCTACTGTCTAAGTGCT | 2569 |
| 2120 | CGCAAGGTCCTCTTCACCAC | 2570 |
| 2121 | GGGGCAAGCGGCTGCGCGCG | 2571 |
| 2122 | AGAGGCATGTGGCCAGCAGC | 2572 |
| 2123 | CTGCTAGCTCAGGCTCTTGA | 2573 |
| 2124 | GGTGCCAGGCCTCCGTGGTG | 2574 |
| 2125 | CACCCACTGCATCACACCCA | 2575 |
| 2126 | AATAGCCCAAAGAATCTCAA | 2576 |
| 2127 | TACTAGGTTGACCCTAACCA | 2577 |
| 2128 | CCACTATCAGTGGGTAGATG | 2578 |
| 2129 | TGCCATAAGCCTTCACCTTA | 2579 |
| 2130 | CCAGACCCACCTGGTCCTGT | 2580 |
| 2131 | CCTAGTCCCCCTGGCCCTCC | 2581 |
| 2132 | GGACCACTTCCTCATTCATC | 2582 |
| 2133 | CCTGATTCTCGTGGTCTTCC | 2583 |
| 2134 | TCCGTTACCTCAGCAGCCGC | 2584 |
| 2135 | GCTATGACTCTTGAGACTTG | 2585 |
| 2136 | TGATCCTGCCGGAGGCGTAG | 2586 |
| 2137 | CAACCCCACCAGCTGCTTGT | 2587 |
| 2138 | ACGATATGTCGCCAGATCAA | 2588 |
| 2139 | CCTTATTCCAATCCCAGGTA | 2589 |
| 2140 | CATGCAGCCTCTGTCCAACA | 2590 |
| 2141 | GCTAGTTCACGCCCGCGCCC | 2591 |
| 2142 | CCAGACCCTCCTGGACCTCC | 2592 |
| 2143 | CTACCTGCACATCTGGGTGC | 2593 |
| 2144 | CTGCTAGGGCATCATGGCAG | 2594 |
| 2145 | TCCTTCTGAGCTCGCTGCTG | 2595 |
| 2146 | AAGGCAGCGTGGCACTAGGA | 2596 |
| 2147 | ACCCATCTCCACTGGGGTTT | 2597 |
| 2148 | CAAGACCCACGTGGTGACAA | 2598 |
| 2149 | AAACCCAGCCCCAGCTCCCA | 2599 |
| 2150 | CCTTCACCCTGACGCTCTCC | 2600 |
| 2151 | TGATCACTGTGAGGCTCCAT | 2601 |
| 2152 | AGGTTATCCACAGGTCCTTG | 2602 |
| 2153 | GGTGCACGCAAACACCATCT | 2603 |
| 2154 | CGCAGCCATGGAGGGTGATC | 2604 |
| 2155 | CTCCCAGCAGCCCCTGGGAA | 2605 |
| 2156 | TGCTAGGCATGGACTGGGGC | 2606 |
| 2157 | TCTTAGAAGTCCTGGTCCAA | 2607 |
| 2158 | CTCTAGGGAGGAGAAATCCC | 2608 |
| 2159 | TCTTATGCAATGAACATCAA | 2609 |
| 2160 | CATAGTTGCAGCCCAAGTCA | 2610 |
| 2161 | CCCTCAAGGACGCCGACCTG | 2611 |
| 2162 | TCTAGGTCATTCCAACCCCC | 2612 |
| 2163 | TTCCTAGGAGAGATGGATGG | 2613 |
| 2164 | GCAAGTGATGGCGACCTAGT | 2614 |
| 2165 | GGCCTAGCTGCCCTGTGAGC | 2615 |
| 2166 | CCGGGACCAGCCTGCAGGAA | 2616 |
| 2167 | CAACTATAATCCAGCTCCAG | 2617 |
| 2168 | CTGACCGCCCTCGGCGTCCG | 2618 |
| 2169 | GTCGTTCCACAGCCGTCTGG | 2619 |
| 2170 | AGACTAGAAACGCTCAAATA | 2620 |
| 2171 | CGCTGATGCATTCATCCAGG | 2621 |
| 2172 | CATAGCGATCCGCGAGAGCG | 2622 |
| 2173 | CCTGGTCAGCCAGGGGTACC | 2623 |
| 2174 | CGTACTCGATGGGGTACTTC | 2624 |
| 2175 | TCAGGCTAGGGGGACAGGTG | 2625 |
| 2176 | CTATTGCAATCCCTTAAGCA | 2626 |
| 2177 | AGCCGGACTTACAGTCACAG | 2627 |
| 2178 | GCAGAGGCCCCAGGACTTAG | 2628 |
| 2179 | CTTACAGGCCAGCCTGCCTA | 2629 |
| 2180 | GAATGATTCTTCACCAAGGT | 2630 |
| 2181 | GGTGGCCTATGGAGTTGCTA | 2631 |
| 2182 | AAAGATGAAGGAGGCCCTCC | 2632 |
| 2183 | CCGCTACAGCTTGTCCTGAG | 2633 |
| 2184 | CCACTACCTGCTGGTGACCC | 2634 |
| 2185 | GCCGCACCACTACGACGACC | 2635 |
| 2186 | CTTACCTGCCTGACACTTGC | 2636 |
| 2187 | CACGTAGCTTGCGCGGCGCG | 2637 |
| 2188 | GGTTAGGCAATACTGCCTTT | 2638 |
| 2189 | TCTGGAACCCAGGGGATCAC | 2639 |
| 2190 | CCTGATGCCCCTGGTGAAAA | 2640 |
| 2191 | CACCAGAAGCACTACTGACC | 2641 |
| 2192 | GTTAGGGGCCCAGAAGGTTC | 2642 |
| 2193 | GTGAGTCTTCCAGGCCTCTC | 2643 |
| 2194 | GGCATCTGGACTCTGTCACC | 2644 |
| 2195 | GTCAGTCCTGCTGGCCCCAA | 2645 |
| 2196 | GGAGCTAAACAACAAATGTT | 2646 |
| 2197 | GCCTGACCCTCGGCCATCGC | 2647 |
| 2198 | TGTTTTATTCCTTGCCCGTC | 2648 |
| 2199 | CTCACATCCAGATTCACCAG | 2649 |
| 2200 | CTCTCAGGAGATCTGAACGA | 2650 |
| 2201 | GAGGACCTCTTTTTCACCAA | 2651 |
| 2202 | CTTGAGGCGGCCGGGCCCGG | 2652 |
| 2203 | GCAGGTAGGCGATGGCCTCG | 2653 |
| 2204 | GTCTCATGCCTGCTCATGGT | 2654 |
| 2205 | GTCCTAGCCATACCACCTGC | 2655 |
| 2206 | CCTGATGTCAAAGGAGAAGC | 2656 |
| 2207 | GCGCTCCAGGTGTGCCGCCG | 2657 |
| 2208 | GGCGCCCTTATGCATTCTCG | 2658 |
| 2209 | AAACTACAGCAGCAGCCTGC | 2659 |
| 2210 | GTGTTCATGCAGCTGAAGTA | 2660 |
| 2211 | CAGCTAGATTACATGCTTCC | 2661 |
| 2212 | TCCATACGTGGCAGGCGTGG | 2662 |
| 2213 | GGCAGAAAGGCCAGGAGAGA | 2663 |
| 2214 | AGAAGGGCTCCTGGTGAGCG | 2664 |
| 2215 | CATGCAGTTGACCGTATAGA | 2665 |
| 2216 | ACACTAGAAGACTGTCAGCA | 2666 |
| 2217 | GGACTAGACATCTTTTAACC | 2667 |
| 2218 | GAATATCCCCCCAACTTCAC | 2668 |
| 2219 | GTCCACGGGCTACACCAAAC | 2669 |
| 2220 | ACTGACGTGATTGGACTTAC | 2670 |
| 2221 | TTCTTGATGGAAAGATGGGA | 2671 |
| 2222 | GGTTGACCTTGGGATTGAGG | 2672 |
| 2223 | CTACTGCAGCTTCTGCCAGG | 2673 |
| 2224 | GCCGGAGCTCCCAGGAGAGA | 2674 |
| 2225 | CGGCTTCCACCTCAGGCTCG | 2675 |
| 2226 | TGCAGTTGGCGTGGCTGAGC | 2676 |
| 2227 | CACCCACATCCCCCTGCAGA | 2677 |
| 2228 | TCCTCAGGGTCCCTCCTGGC | 2678 |
| 2229 | GCCCCTGATCCTTGCTGTGG | 2679 |
| 2230 | AGCCCCTCTAGCCATGCCAT | 2680 |
| 2231 | CCATGACCAGTGCAGCTGTG | 2681 |
| 2232 | AGAGCTAGCATTCAGACCTC | 2682 |
| 2233 | GCTGCAGCAACAACGGTTTT | 2683 |
| 2234 | GACCCTACTGCTGTTGCTGC | 2684 |
| 2235 | CCTGATCCCCAAGGTGTCAA | 2685 |
| 2236 | CAACCGCAAGAAGATGACCC | 2686 |
| 2237 | TGCCCACAACCTCCTGACAG | 2687 |
| 2238 | TGTGACCAGCTTTCAGGCAG | 2688 |
| 2239 | CTTAGTCTCCACCTGGATGC | 2689 |
| 2240 | CATCTAACCTGGCAGCTGGA | 2690 |
| 2241 | GAAGAGGCTGATGATGCTGG | 2691 |
| 2242 | TCACTTCCAGAAAGGCAGCA | 2692 |
| 2243 | ACACCCCGGCCTAAGCAGCG | 2693 |
| 2244 | CAGCCACTGCTTCTGGCCTC | 2694 |
| 2245 | AATCCATGTCTGGGCAGGGA | 2695 |
| 2246 | GATCAGACCCCCTGGGCTTC | 2696 |
| 2247 | CGAGGACCCCAGCGACCCTC | 2697 |
| 2248 | ATGCAAGTGAAACGGCTACG | 2698 |
| 2249 | GACCGAGGCGTGTCTCCAGC | 2699 |
| 2250 | ACGCCTGTAGTATGTTATGC | 2700 |
| 2251 | GTGGCAGCTAACTTTCCTTC | 2701 |
| 2252 | CCTAGGCAGGGGGTGGCTCC | 2702 |
| 2253 | GCTCACCTTCGGGATCAGCT | 2703 |
| 2254 | CCGTAGTCCTCCTGGTGCTG | 2704 |
| 2255 | TGAACCTACTCATCCACATT | 2705 |
| 2256 | CGGGATCTTGCAGGACCACC | 2706 |
| 2257 | GGCCATGCGGGAAAGAGCAG | 2707 |
| 2258 | CCTCCATGATGTTGATGCCA | 2708 |
| 2259 | TCACCACAGTCACCCTGGCG | 2709 |
| 2260 | CACCTGCCAGGCCCTGGGCG | 2710 |
| 2261 | CCAGATCTCCCTGGAACTCC | 2711 |
| 2262 | CAGGGAACCCAAGGGCTACA | 2712 |
| 2263 | CGGCTAGAAGTTCGAGAAGC | 2713 |
| 2264 | TTTCCTAGATCACCTCCAAC | 2714 |
| 2265 | TCCTACTCTGGGTCCTCCTC | 2715 |
| 2266 | TCAGCCTGCTGGCGGGTACG | 2716 |
| 2267 | GGGGGATCAGATAGGCCTGG | 2717 |
| 2268 | GAGGCAAAGCACCTCTCGGA | 2718 |
| 2269 | TATGGAGCGCTCAGCAGCTG | 2719 |
| 2270 | CAAGTCCTCAGAAATCCATC | 2720 |
| 2271 | TGATGAAAACAATGGTGCTC | 2721 |
| 2272 | AGCAGCCTCCTGCTCTACAA | 2722 |
| 2273 | GGGCTCAGTGGCCCACGGTC | 2723 |
| 2274 | CACAGGCAGCTTCAGGAGGC | 2724 |
| 2275 | CCTGAATCAGATGGTCTTCC | 2725 |
| 2276 | TGGTTCTTGATGTCCTTAGT | 2726 |
| 2277 | AGAGTATGCTCCTTTCTGCC | 2727 |
| 2278 | AGCTGCACAGCAGGGGCAGG | 2728 |
| 2279 | CGACTCAGCCAGCAGCACCA | 2729 |
| 2280 | TAGGAGAAGCCCTGGCCCTC | 2730 |
| 2281 | TTTCACTAGGGTTCACTTGA | 2731 |
| 2282 | CAGGTACACCCAGAGAGGCA | 2732 |
| 2283 | TTTCACTGCGCTCATCATGA | 2733 |
| 2284 | AAACTAGAGGGCTCCATGAT | 2734 |
| 2285 | CGGTAGCGCTGTCAGCGGCG | 2735 |
| 2286 | TGTTCCTAGCCACCTGGGGC | 2736 |
| 2287 | GCTCTGCTAGGGGGCGCTGG | 2737 |
| 2288 | CTGGAGAGTGGTCTCTGTGC | 2738 |
| 2289 | ATGGCATGGCGAAAAAGTGG | 2739 |
| 2290 | TGTGCTATGAAGGGGGTGTG | 2740 |
| 2291 | CAGCAGCCGGGATGCCGGCG | 2741 |
| 2292 | GGAGCATGAGGTAATCAGCC | 2742 |
| 2293 | CGCCCACCGCAGCAGCTTCA | 2743 |
| 2294 | ACACTCATGTATCTTCATTC | 2744 |
| 2295 | CACAGCCAGGGCTGGAGGTG | 2745 |
| 2296 | CTGTATGGAGGCTCCATCAT | 2746 |
| 2297 | GCAGTACCTGGCCATGGGCT | 2747 |
| 2298 | CCCCACCCCGGCAAGGCTGG | 2748 |
| 2299 | TAGCCCTATGACTTATCCTG | 2749 |
| 2300 | TGAGCTGCTAGTCCCAGCTG | 2750 |
| 2301 | CGTTGAGCACGGTCCCAAAG | 2751 |
| 2302 | ATCGACAGGCGCATTGTGGA | 2752 |
| 2303 | CACCTCAGTCTGCAGCTACA | 2753 |
| 2304 | CCTGATCCTCTTGGCATTGC | 2754 |
| 2305 | TGCTATTTCCGGACCTAACA | 2755 |
| 2306 | ACTCCCCACAGAGGTCCAGC | 2756 |
| 2307 | GACCCCTAGCTGCCTTGGAT | 2757 |
| 2308 | ATCCATTCCCGTACTTCCTT | 2758 |
| 2309 | TGCCTTGACCAGTGCCCCAG | 2759 |
| 2310 | TGGCTTAAGGGCTGATGTGT | 2760 |
| 2311 | GAGGCTGAGCAGCCACAGGA | 2761 |
| 2312 | CCTGAATCCAAAGGAGAGCA | 2762 |
| 2313 | GATCTACCCGCCCTGAGGCC | 2763 |
| 2314 | GCCAGGTTGCCAGGACTGCG | 2764 |
| 2315 | TGCCCACCGACTCCATACCT | 2765 |
| 2316 | GATCGCAGCCCGCCAGGTCC | 2766 |
| 2317 | GTCCAGGGAGGCTGCGCCTC | 2767 |
| 2318 | GCGCATGGCCGTGGAGGAGG | 2768 |
| 2319 | CCTGATTCCCTGAGGACCAG | 2769 |
| 2320 | CACTCACTTCTTCAGGGCAG | 2770 |
| 2321 | CCCAGTTCTCCTGGCCAGAA | 2771 |
| 2322 | CTGGATCATCTTCTCGCGGT | 2772 |
| 2323 | GATATGGGGCCCAGGATGAC | 2773 |
| 2324 | GCACGTGGCCTCGTAGCCCA | 2774 |
| 2325 | TAGCCAGGAGCACGATCTGT | 2775 |
| 2326 | TCCACCGACCACCTCCAGCC | 2776 |
| 2327 | TATCTAAGCCCGGTAGCCAC | 2777 |
| 2328 | AAAGAGCCTAAGGGTGAAAA | 2778 |
| 2329 | TGTGCAGTCGAACAGCATGA | 2779 |
| 2330 | CGTCTACAAGTGAGCGGCCC | 2780 |
| 2331 | AGCCCTACTGCACCAGGGCC | 2781 |
| 2332 | TAGGCCCTAGGGTGGCTCTG | 2782 |
| 2333 | CAGCGTGAAGGCTACTGCTC | 2783 |
| 2334 | CCTGAGCCACCTGGTGCTGC | 2784 |
| 2335 | CGTGATTTCCCTGGAGACGC | 2785 |
| 2336 | ATCCATGCCCGTCAGGTAGT | 2786 |
| 2337 | GTTTCATGCCTGCTCATGGC | 2787 |
| 2338 | TGCGCTAGACCAGGGGGTTC | 2788 |
| 2339 | GAGTTAGGTGCCAAAACTTG | 2789 |
| 2340 | CGTAGTGACCAAGGTCCAGT | 2790 |
| 2341 | TCTAGCTCTCAGCCTGCTAC | 2791 |
| 2342 | TCGCTAAAGACCTATTTCTA | 2792 |
| 2343 | CACTCAGAACCTTAGGCATT | 2793 |
| 2344 | CCTGATGCTGCTGGACGGAC | 2794 |
| 2345 | AGCCTAGGCCAGGAGACCTA | 2795 |
| 2346 | GAGAGTAGGCAAATGACCTG | 2796 |
| 2347 | CGTAGTCCTCGTGGTGACCA | 2797 |
| 2348 | CCAGCCTAGGTGAACATGTA | 2798 |
| 2349 | TTCATAGACGGCCACCTAGA | 2799 |
| 2350 | AGCTTCTATGTGTCCTCTTT | 2800 |
| 2351 | GAGGCTCACTCCTCTGTGAT | 2801 |
| 2352 | CCTGATCTGCAAGGAATGCC | 2802 |
| 2353 | CCTAGGGGGAGGACGAGGAG | 2803 |
| 2354 | CGGAATGCTGAGCGGTGTGG | 2804 |
| 2355 | CTTCCCTATCTCTACAGCCC | 2805 |
| 2356 | GTCCAGTCCCCAGGAGGAAA | 2806 |
| 2357 | CAGCCACCAGTCATAGGGGG | 2807 |
| 2358 | TGGTGTTAGGTAAATCCGGG | 2808 |
| 2359 | CTCCAGAGTGCATAAATCAG | 2809 |
| 2360 | AGGTTAGCCGTCAGCACCCT | 2810 |
| 2361 | TGAGGCTGCGCGGGCCCTTG | 2811 |
| 2362 | GATCTAGGCATCAATAATCA | 2812 |
| 2363 | ACGGGCCCAGGGGCGCGGAC | 2813 |
| 2364 | AAAGTGTAGATCTATCCCGA | 2814 |
| 2365 | CAGATGCTGGGAGTCCTGCC | 2815 |
| 2366 | CTCTCAGCTCTGCCTTGGTC | 2816 |
| 2367 | TGACCTCACCTGGGTGTTCG | 2817 |
| 2368 | GGCCTATTCCACCTGTCCCC | 2818 |
| 2369 | GTATCAGACAGATGAAGTAG | 2819 |
| 2370 | GGAGCACCTGGCCAAGCTGC | 2820 |
| 2371 | CGCTCGTACTCCGTGCCCGA | 2821 |
| 2372 | CTCACTTGCCGGCCTTGATC | 2822 |
| 2373 | TGGCTCCAGTATCGTCAACA | 2823 |
| 2374 | CCCAGACCTGCCAGGCTGCA | 2824 |
| 2375 | AAGGCCCAGCTCAACAGTCA | 2825 |
| 2376 | ACTAGTCCTATTGGTCCTCC | 2826 |
| 2377 | GGATCCGCACGGGCCGGGTC | 2827 |
| 2378 | CTGGCAGCAGGTATCACACA | 2828 |
| 2379 | CCGGGACGACCCCGGCTTTG | 2829 |
| 2380 | AGCTGACCCGCAGGGTGATC | 2830 |
| 2381 | ACTTTACAGATCAGAGGTGG | 2831 |
| 2382 | CCTGACTGGACAGCCACCAC | 2832 |
| 2383 | AAGAGGCATCCATTTCAGGT | 2833 |
| 2384 | CATAGTGAGTTTGGTCTCCC | 2834 |
| 2385 | CAGAGCTAACACAGTCTGAA | 2835 |
| 2386 | AGCATGAGCTCTGCCTTCTC | 2836 |
| 2387 | CAACCACCGGGACGTACGGC | 2837 |
| 2388 | AATGCAGTCCCCAAGGAGGT | 2838 |
| 2389 | TGTTTAGCTGGAAACAGACC | 2839 |
| 2390 | CTCTGCAGGAAAAATGGTGG | 2840 |
| 2391 | AATGAGGAAGCTGGATCTGC | 2841 |
| 2392 | TCCTCAGTCTTCCTCATTCA | 2842 |
| 2393 | GGCCGGTCAGTCAGTCTTAC | 2843 |
| 2394 | GGTAGGCTAGCTCGCCGCTT | 2844 |
| 2395 | GGCTACTGCTGCTGCGGCGG | 2845 |
| 2396 | AAACAAGGGGATGCCTGTGA | 2846 |
| 2397 | GTGACCATGGCCTGCGAGGA | 2847 |
| 2398 | GTCTCAGGCGTCCCTCCAAG | 2848 |
| 2399 | CGACTGTGCGTGGCGAGCAG | 2849 |
| 2400 | CGAGGGCTGTGGACGCCCTG | 2850 |
| 2401 | GGCCTTCTGAGCAGAATGGA | 2851 |
| 2402 | CCAGATCCCATTGGACCACC | 2852 |
| 2403 | CATTGGCCTATTCCCGGCGC | 2853 |
| 2404 | CCGCGTCAGGTGCCAGCACA | 2854 |
| 2405 | CAGCTAGGCAGCCACGTAGA | 2855 |
| 2406 | ATGTGATGATGTCCACCGCG | 2856 |
| 2407 | AGTCATCTCTCTGTGCAGTT | 2857 |
| 2408 | GTCTCATCCAGGGGAACCTT | 2858 |
| 2409 | GCGTAGGAGCAGTCAAGAGA | 2859 |
| 2410 | AGACTAATGCCTCATTTGTT | 2860 |
| 2411 | AGAGATGGAGCTGGTCCCCC | 2861 |
| 2412 | GGCTAACCCATCTCTCCTCG | 2862 |
| 2413 | AACGATCTCAGTGGAGAACG | 2863 |
| 2414 | CCCTCAGGGCAGGCTGAGCG | 2864 |
| 2415 | GCCTGCCCATCTGCTGAACC | 2865 |
| 2416 | CAATCATTGGGCAGGTGGAG | 2866 |
| 2417 | CCTAGAAAGCCAGGCCTCCC | 2867 |
| 2418 | GCACTACTTGGCAACCTCCT | 2868 |
| 2419 | AAGGCTAGTGGAGACCTGGG | 2869 |
| 2420 | AGATTCATTGGTGCCGAGGG | 2870 |
| 2421 | GGCAACGGCGGCAGTGGGCG | 2871 |
| 2422 | CTTTGACCGGTAAGTAGGAG | 2872 |
| 2423 | CCTCACAGGCCACGCTCTCC | 2873 |
| 2424 | CAAGAAATGCCTGGAGAAAG | 2874 |
| 2425 | CCTAACTAACACTGGATCCC | 2875 |
| 2426 | TCTTTATCTTCCTCCAGTGC | 2876 |
| 2427 | GCCAGCCCTCCTGGGGCCCG | 2877 |
| 2428 | TGGCAGGCAGCGGCAGTTGT | 2878 |
| 2429 | AACGATGATAAAGGTCATGC | 2879 |
| 2430 | CATGTCTATCAAGTCAGAAC | 2880 |
| 2431 | GGACTAGATTCTCAGAGCTC | 2881 |
| 2432 | ATTCAGCGGGGCACGACAAA | 2882 |
| 2433 | CCTCCAAGTGTAAGCGGTGG | 2883 |
| 2434 | GACGCACTGCAGCTCGGCCT | 2884 |
| 2435 | AAGCCACCTTGTTGTTAGGA | 2885 |
| 2436 | AAGTTCAACTCTGGTTTAAA | 2886 |
| 2437 | CCTAGCCCTCTGCCAGATTT | 2887 |
| 2438 | GAGCATTCCTCCTTGTTATC | 2888 |
| 2439 | AGGGCCTCTACAGTGGCGTA | 2889 |
| 2440 | CCTGATCCTGTCGGTCCAGC | 2890 |
| 2441 | GCAGCTGCAGGCTGCGCACC | 2891 |
| 2442 | GCTAGGGGCTGAAGTCCCTC | 2892 |
| 2443 | GAGCTAGAAGATCCTCGCCA | 2893 |
| 2444 | AGAGCATTTCTTGAATCCAG | 2894 |
| 2445 | GGCCCAGACCCCATAGCCAA | 2895 |
| 2446 | GGGACAGCCGCCTGACCAGC | 2896 |
| 2447 | AGACATACCTCTTGTCCTTG | 2897 |
| 2448 | CAGGCGAGCAGCATGGTGTC | 2898 |
| 2449 | TGACACCCACAACATGTCAG | 2899 |
| 2450 | AAGTGTTAAAGAGGCTTTGC | 2900 |
| 2451 | GGCCTGAGTGCGGGCGCGGG | 2901 |
| 2452 | GCGGCTATGCTGGGAGCATG | 2902 |
| 2453 | GCTGATCCTGCTGGTCCTGC | 2903 |
| 2454 | CCACATATTTGTGCTGGAGC | 2904 |
| 2455 | CCCCCAGAGAGAGAGTGGTG | 2905 |
| 2456 | GAGGTATGTCTCCAGCAGGT | 2906 |
| 2457 | CAAGATGCGTGCTCTGGAGG | 2907 |
| 2458 | TGCGAACTTTTCACCACCAT | 2908 |
| 2459 | GCTAGTGCTCCCGGTCCTGC | 2909 |
| 2460 | TGGTAGCTGGACAACAAAAA | 2910 |
| 2461 | CCATCAGTGACGGCCTGGGT | 2911 |
| 2462 | GTCTCATGCCTGCTCGTGGT | 2912 |
| 2463 | GGCTTCATGCCCATGTATGT | 2913 |
| 2464 | CTCACGGCCCTGGCAGTCCT | 2914 |
| 2465 | CTGGCTAGAGGCGAGGCTTC | 2915 |
| 2466 | GATGATCCCCCAGGTCGCGA | 2916 |
| 2467 | CCTCAGGGGCCAACAGGTCC | 2917 |
| 2468 | CGGCTACAGATGCCATTGAG | 2918 |
| 2469 | GAAATGGTTCTCCTTGCTTG | 2919 |
| 2470 | GTACCTTCAGCTTGGAAGTC | 2920 |
| 2471 | GCTGCAGGTCCCCCCGGCCC | 2921 |
| 2472 | CATGATGATCAAGGTGCTCC | 2922 |
| 2473 | CCCTGACCCTCAGGGTGTCA | 2923 |
| 2474 | GCCTATCTCCTGGGTTCCCG | 2924 |
| 2475 | ATGGAGAGATTGATCCTAAA | 2925 |
| 2476 | AGTCTAGGAGAATTTACTAC | 2926 |
| 2477 | ATCCCTTAAAAAGCATTTCC | 2927 |
| 2478 | CGCAGTCCCCCAGGTGAGAG | 2928 |
| 2479 | GTCTCATGCCTGCTTGTGGT | 2929 |
| 2480 | TGGGCTATTGGGGGCCCAGA | 2930 |
| 2481 | TGGCTATGCCACCAGTAGCA | 2931 |
| 2482 | CCTGGTGACTGTGCGTTCTG | 2932 |
| 2483 | CACCACCGTGCTGGGCAATC | 2933 |
| 2484 | TATCTAGCGGAAGGCCTCTG | 2934 |
| 2485 | AAGAGACGAGCCAGCGCAAG | 2935 |
| 2486 | CCTGAACCTCCTGGTGCCCC | 2936 |
| 2487 | AATGATGCTCCTGGACTGCG | 2937 |
| 2488 | TGAACCTACTCCACGCCCAC | 2938 |
| 2489 | CCTGCTAGTCCTAGGGTAGG | 2939 |
| 2490 | TTCAGGAGCTTCAGTTGTTC | 2940 |
| 2491 | CTGCTATCTGCTTGCATTCG | 2941 |
| 2492 | TCCTACCGCTCACTCGGCAG | 2942 |
| 2493 | CTGACTCCAGCTGTCGTCAC | 2943 |
| 2494 | CCTGAAAAGCCTGGTATTCC | 2944 |
| 2495 | ACCTGATCCTCATGGCCCCG | 2945 |
| 2496 | GATCACCTCTCAGAGTCCTC | 2946 |
| 2497 | ACTCCATGACAGTGTAATTT | 2947 |
| 2498 | CTGCTAGTCCAGGGGGCTGG | 2948 |
| 2499 | CTGCCAGACTGCATCCAGGC | 2949 |
| 2500 | TCTAGGAGCCTCTGTTTACT | 2950 |
| 2501 | GTGTTCCAACCTGAGAATGC | 2951 |
| 2502 | CGAGATATGATGAAGGAGAT | 2952 |
| 2503 | ACAAGCCCCGTTGGAGCTGC | 2953 |
| 2504 | GTCCCAGAAGGAGGCCCAGC | 2954 |
| 2505 | GGCCTCGAGTCAGTTCGAGT | 2955 |
| 2506 | CCAAAGGCTACATTTCATGA | 2956 |
| 2507 | TTATCCGGGAGCCCCCTGTA | 2957 |
| 2508 | TTAAGGAGCACTGGAACCGG | 2958 |
| 2509 | CTACAGTCCCCTCATCCAGC | 2959 |
| 2510 | CGGCGTTGCACTGGCACACT | 2960 |
| 2511 | CGGGCACATTGTGGAGGGCT | 2961 |
| 2512 | TGACGTCGTGGTGAGCAGCT | 2962 |
| 2513 | GATTCCCAGTTATGTCCAAT | 2963 |
| 2514 | TGGTGGGAACATCTGGTGGA | 2964 |
| 2515 | TTCATCCAAGTAATGGCATC | 2965 |
| 2516 | GCGGGGAGTGGCTGGGGGAC | 2966 |
| 2517 | GATGAACGCGATGTAGCTAT | 2967 |
| 2518 | AGAAGGGCTCCGGGTGAGAA | 2968 |
| 2519 | CTAGCTGGACTCCGGACCTG | 2969 |
| 2520 | AGAGGGGGCTGCAGGGCCAG | 2970 |
| 2521 | GCTCGCCCACCTGCTGGCCC | 2971 |
| 2522 | AAGGATTAGCCCCACAGATG | 2972 |
| 2523 | CAAGGACAACACTGATCGCC | 2973 |
| 2524 | AAGTAGATGCTGCTGTCAGA | 2974 |
| 2525 | TCCCACTAGATCTCCTTCCT | 2975 |
| 2526 | GCATTCTCCAGGAAAGCCGA | 2976 |
| 2527 | TTCTGCCAATGTGAAATTAA | 2977 |
| 2528 | GGCAAGGGTCTTCTACATGT | 2978 |
| 2529 | CGCCAGGTATTTCGGGGTGC | 2979 |
| 2530 | AGCTGAACATGGTGCAGAAC | 2980 |
| 2531 | GGGCTACAAGTCACCACCGT | 2981 |
| 2532 | GAGCTGTCACACATCCAGAT | 2982 |
| 2533 | TCCAGTCAGTAAAGAGTAGA | 2983 |
| 2534 | ACAGAGCTAGCCGCCCCAGT | 2984 |
| 2535 | GCCCCGCCATTCTCCACGAT | 2985 |
| 2536 | ATGAGGCCTCCAGGGCTTCC | 2986 |
| 2537 | AAGGACGACCGTGGAGACCC | 2987 |
| 2538 | GCATGTCCCAAAATATATTT | 2988 |
| 2539 | GTTACGTGACAAAATTCTGC | 2989 |
| 2540 | TGACCGACTACAGCAGGTGC | 2990 |
| 2541 | CCTTGGGCATGGTGTGCGGG | 2991 |
| 2542 | GTCTCTGTAGATGATTGACT | 2992 |
| 2543 | GAAGAGTACAGAAAGAGAGA | 2993 |
| 2544 | GGGTAAGGGCATGACGCTGA | 2994 |
| 2545 | GCGTCCAGCCCTGCACGTTC | 2995 |
| 2546 | CGAGCCGCCCAGCGAGCTCA | 2996 |
| 2547 | GTTGAAGAAGCACTTGATCT | 2997 |
| 2548 | CTAGCTGGGACTGAGAGACC | 2998 |
| 2549 | AAAAGAGAGCCGTGGGGAGA | 2999 |
| 2550 | CTCTTTCAGCCCAGGCCCTC | 3000 |
| 2551 | CGGGATAATGACGGTGCTCG | 3001 |
| 2552 | GGTTCCACTAGTAGTGCTGG | 3002 |
| 2553 | GTTTCCTCAGACAGCAGGTG | 3003 |
| 2554 | CCTTACATGCCCTGGTAAGT | 3004 |
| 2555 | GGCCAGCTAGTCGCGTTCGG | 3005 |
| 2556 | CGCTTCCTGATGCTGGGCCC | 3006 |
| 2557 | CCTCAATTCTAGAAAGGCAG | 3007 |
| 2558 | CCGGGAGCACACGGAGGAGC | 3008 |
| 2559 | CCTGACATGATACTGCTTCC | 3009 |
| 2560 | CTACTCCCACAACAAGGCTC | 3010 |
| 2561 | CTCAAGGACCCTCTTGTCCG | 3011 |
| 2562 | ATCAGCCTTTACCAACTTGC | 3012 |
| 2563 | CCTGAAGCCCCCGGACCACC | 3013 |
| 2564 | GTATTTACTGAGTTCCCCAC | 3014 |
| 2565 | TGCATGCTAGCTGCACATAT | 3015 |
| 2566 | GGAGGGCAAGGAGTGCAGGT | 3016 |
| 2567 | TCCAATATGCTGAGAGGCAT | 3017 |
| 2568 | CAGCAAGGTGGCCACACAGA | 3018 |
| 2569 | GTAAGTGCAGTTGGTCCCCC | 3019 |
| 2570 | ATCACTGTTCAATTTCCTGT | 3020 |
| 2571 | GGAAGTGCACACCTGACAGG | 3021 |
| 2572 | CAAAGTCCTCCTGGTCCCAG | 3022 |
| 2573 | TCCAAGAGGCAAATTTCCAG | 3023 |
| 2574 | ACTGACACCCTATATCCCCA | 3024 |
| 2575 | CGATGCACTACAGCAGGGCC | 3025 |
| 2576 | CTCAATAGGCACGAGCAGAC | 3026 |
| 2577 | GTGCAACATGGCTCGCATTG | 3027 |
| 2578 | CGGGTACAGGAGGGCTCTGG | 3028 |
| 2579 | GATCCTCACCAGGGAGGAGG | 3029 |
| 2580 | CAGCAGCCTAGATCATTGCC | 3030 |
| 2581 | GCAGGCCTCCGGAATCACCC | 3031 |
| 2582 | GGACTGCAAAACCAAACCAA | 3032 |
| 2583 | CTCAAGGCCGCCGTCTGCGC | 3033 |
| 2584 | CCCACCTCATGATCGTTCTG | 3034 |
| 2585 | ATGTCTAAGTGAGTAGGCAT | 3035 |
| 2586 | ATTGCTTTTACCTAGTGCTA | 3036 |
| 2587 | CTGGCTTCTAGGTTTCTGCT | 3037 |
| 2588 | CAAGCGGGACAAGGCCCACG | 3038 |
| 2589 | GGAGGGCTAGCCGGCCGGCC | 3039 |
| 2590 | CAGCAGCACCTTCTGTGAGC | 3040 |
| 2591 | CCTCAAGGCCTGGGTGCTGC | 3041 |
| 2592 | TACGCCTCCAGCAGGACCAC | 3042 |
| 2593 | TGGGAAGCTAGAGCCACACC | 3043 |
| 2594 | TCTTTCCCACACGGCCGTCC | 3044 |
| 2595 | TGCAGGTTAGTTCCTGTCCC | 3045 |
| 2596 | TTCTAGAATGATGACGGTGA | 3046 |
| 2597 | TCTCAGCGTTTATCACTGTC | 3047 |
| 2598 | AGCTACAGAGAGCTGGGCTG | 3048 |
| 2599 | GATCTCCAGCTGTGCAAACT | 3049 |
| 2600 | ACGACCAGGTATGGTGCGGC | 3050 |
| 2601 | CCACATACTGTCCGAGCTGG | 3051 |
| 2602 | AACAGCATCTGGGAAAGTAG | 3052 |
| 2603 | CCATTGGAAACAGCGCATGG | 3053 |
| 2604 | CGAAGCGGCCCCGGGCGCGA | 3054 |
| 2605 | AGAGAAGCCCCTGGATGGCC | 3055 |
| 2606 | ATTAGAAGAGTAGGAAGATT | 3056 |
| 2607 | CTCTTCCTCATATCTCCACA | 3057 |
| 2608 | CTACTTGTTGTCCCGGTAGA | 3058 |
| 2609 | TTCCACAGAGCATGTCTCAG | 3059 |
| 2610 | GTGACCACTGATCGGAAACG | 3060 |
| 2611 | ACTATAGCACAGCTTTATCC | 3061 |
| 2612 | AGACACCATGAAAGCTGCCA | 3062 |
| 2613 | CGGCCAGATGACCATCCAAT | 3063 |
| 2614 | AAAGCTGAAAAGGGACGAAC | 3064 |
| 2615 | AAGTTAGCAAACCAGGAGAA | 3065 |
| 2616 | CCAAGGCCTGATGGTGAACC | 3066 |
| 2617 | CAGCTACAGCTCCCTGCCAT | 3067 |
| 2618 | ATAAAAGAAGGCCAGGACAT | 3068 |
| 2619 | CTCAGGAATCAGATGCACCA | 3069 |
| 2620 | TCACCTCTTGGCAGCTCTTT | 3070 |
| 2621 | AACAGCCAGCTGGGGTAGAA | 3071 |
| 2622 | GTCACTGGTTCTCGTGGTCC | 3072 |
| 2623 | ATAATCCTAGTTTACTTCAG | 3073 |
| 2624 | CGCAGATCTGATACTCAAAG | 3074 |
| 2625 | GCTACCAGAAATGCCGAGCC | 3075 |
| 2626 | GCGTGAGCTACGGGTCCCAC | 3076 |
| 2627 | AATCTGATTCCAGAACAGGA | 3077 |
| 2628 | GGACCCCCTGGCCGACTACC | 3078 |
| 2629 | TGGAAGCCCTGAGGGTGGAG | 3079 |
| 2630 | GATGGCATTTCAAGACTGGT | 3080 |
| 2631 | AATCCCTGTAAAGATATTAT | 3081 |
| 2632 | AGGGCAAGGAGTGCAGGTGA | 3082 |
| 2633 | CAGTGGATACATCTCGGGCA | 3083 |
| 2634 | CCTTGGCTCACTTCAGCAGC | 3084 |
| 2635 | CACAACATGCCCATTTATGA | 3085 |
| 2636 | GCTAGTGAAGTTGGCAAACC | 3086 |
| 2637 | CCCTTCCCAATTGTCTGGAA | 3087 |
| 2638 | TGACTATAAATGGAGTGAGA | 3088 |
| 2639 | CTGAGTTCTCCATGAGTGTT | 3089 |
| 2640 | AGGCAGAAGACTCTGGGTCT | 3090 |
| 2641 | CGAGTGGTGCCGCACGAAGC | 3091 |
| 2642 | CTGACTGAAGTGGACGGACA | 3092 |
| 2643 | GGATGAACAGGGCCCAGCAC | 3093 |
| 2644 | ACTGAAGCACGGGGTCTTGC | 3094 |
| 2645 | ATCAGCCCCGCTGGAAAAGA | 3095 |
| 2646 | AGAAGCCACACAATATCAAG | 3096 |
| 2647 | GTACGCTCTTGAGGTTGTAA | 3097 |
| 2648 | CCACCTTCATGCCAATGTCA | 3098 |
| 2649 | GCTTGGCCAACTTCCCAAAC | 3099 |
| 2650 | ACTCAGAGTTGCCGTATTCG | 3100 |
| 2651 | CTGTCTTTTAGAACAGGATG | 3101 |
| 2652 | CGTCGAGCTTCTGCTTCTCC | 3102 |
| 2653 | TCTTCAAGATATCAAAGAAC | 3103 |
| 2654 | GGGCTAGAGCATCTTTGAGC | 3104 |
| 2655 | CGAGGGGGCCCCTCTCCCCA | 3105 |
| 2656 | GGCTCACAGGGCACAGAGCA | 3106 |
| 2657 | CCACTGGGGCCCCCGAGACC | 3107 |
| 2658 | CCAAGGCCTCGAGGTAACAG | 3108 |
| 2659 | AAAAGTGAACAGGGTCCCCC | 3109 |
| 2660 | TGGTGTTGGATAAATCCGGG | 3110 |
| 2661 | CAGCTCAGTTCGTGGACGCC | 3111 |
| 2662 | TTCACTCCTAGAAGTTCTTC | 3112 |
| 2663 | AGCATGGATGGGATGTGCTG | 3113 |
| 2664 | GATAGGGAGTTGCCAGGAGA | 3114 |
| 2665 | CGAGAACCTGCTGGACCAAA | 3115 |
| 2666 | GGACTTGGCAGCTGCGCGGC | 3116 |
| 2667 | GGTCTTGCTAGTGCTCGGGT | 3117 |
| 2668 | CAGCACCATGACCCAGGTGA | 3118 |
| 2669 | AGAAGCACATGGCTTCATAA | 3119 |
| 2670 | CTCAAGGTCCAGGTTAAATC | 3120 |
| 2671 | CCGTGCTGAGGCTGTTCGTG | 3121 |
| 2672 | GGCTGTCATCACCAATCCCA | 3122 |
| 2673 | GAGAGCAGTCATCTTTCCCC | 3123 |
| 2674 | GCCAAGACCCACTTCAAAGA | 3124 |
| 2675 | AAAGATGAAACAGGTGAACG | 3125 |
| 2676 | TTCACCCTGAGAGACCTCTC | 3126 |
| 2677 | CCCAGAAAAGATGGTGTTCC | 3127 |
| 2678 | GGGCATCCTAGGGAATGTCC | 3128 |
| 2679 | CGCACAAGGAGCTCTACAAG | 3129 |
| 2680 | GCTACACCCAGGGCGCTAGC | 3130 |
| 2681 | TCTACAGCTCTGGGAGGCTC | 3131 |
| 2682 | GTATGGCAAAGGTGCCCTTG | 3132 |
| 2683 | CTGGCAGAAGGCCTCTGTGG | 3133 |
| 2684 | GCTAGGGGTGGTGGCTGTTG | 3134 |
| 2685 | GATCCGGTTCTGGCTCCAGT | 3135 |
| 2686 | ACAGGGCCTTAGTGTATCAC | 3136 |
| 2687 | CAGGATGACCCAGGACTTAA | 3137 |
| 2688 | AGTGCTCCAGTTTACCTAAA | 3138 |
| 2689 | CTGGGAGATGATGCTGATCC | 3139 |
| 2690 | ACCTACACCCTAGCCAAGTG | 3140 |
| 2691 | TGACTAGAAGGACGTCCTGT | 3141 |
| 2692 | CAGAGTGGTAGGAGTGTGGA | 3142 |
| 2693 | CATCCAGTAGATCAAGGAGA | 3143 |
| 2694 | ACTGAATCACCAGGAATTCC | 3144 |
| 2695 | ATCAAGTCTCCGCTGATACC | 3145 |
| 2696 | TAATAACTCAAGCAACCTTC | 3146 |
| 2697 | AGCACTTGATGTGCTTGGCT | 3147 |
| 2698 | GCGCTAGTGGTACGGGGAGA | 3148 |
| 2699 | CACTAGTGGTTGACGAACTC | 3149 |
| 2700 | GATGGACTACTTCAGGCGTG | 3150 |
| 2701 | AACTGCTAGATGCCGTTCAA | 3151 |
| 2702 | GTGAAGTGTCTATCTTTCAT | 3152 |
| 2703 | CACATGGAGAAGATCTTCTC | 3153 |
| 2704 | AGCTGTCTAACAGTTGGCCT | 3154 |
| 2705 | GAGCTAGATCCGGTCCATCA | 3155 |
| 2706 | GGTTCTAATTTGGAATAGGC | 3156 |
| 2707 | CGGAAGGTCTAAGAGATGGT | 3157 |
| 2708 | GTCTAAAGAACACCCCCAGG | 3158 |
| 2709 | GAAGCTAGCGAGTCAGTAAC | 3159 |
| 2710 | CCCAGACTCTACCATTTTTC | 3160 |
| 2711 | GAAACTAGGTGAGGCCGCCA | 3161 |
| 2712 | AAGCAAAGAGAGGCCCTAAT | 3162 |
| 2713 | AGACGAAGTACAACTGAAGG | 3163 |
| 2714 | TCTAAGTGTTGGGTCCGTCC | 3164 |
| 2715 | AATGACTATGCCTACCTCAA | 3165 |
| 2716 | CCTGAACGAGAGCTTTGGCT | 3166 |
| 2717 | CCTAGAGGGAGACGGTGCGC | 3167 |
| 2718 | GGCTAAAAACGGGGTCTCTG | 3168 |
| 2719 | GGACTAGTGAATGCTTCCTG | 3169 |
| 2720 | ACAGAGCTAGCTAACGATCT | 3170 |
| 2721 | CTTGAACTAGCGGTCCCCAT | 3171 |
| 2722 | AGCACTATGCCTTAACAGAT | 3172 |
| 2723 | GTCCCCAAAACCGTTGTTGC | 3173 |
| 2724 | GAAGACTGCAGGTCAGCCCT | 3174 |
| 2725 | TCTCCCCCTTCTCCCTGAAA | 3175 |
| 2726 | GACCCCTGCCAGCGTCATGG | 3176 |
| 2727 | GCACTGCTAAGCGCCAGCCT | 3177 |
| 2728 | AAAGAACTCTGAATTAGGTA | 3178 |
| 2729 | GACCTGCTAGAGGAAGTCGA | 3179 |
| 2730 | GAACTAATGCGCCCTGTTCA | 3180 |
| 2731 | GAGGCACATATAGGTCTTGA | 3181 |
| 2732 | GGGAAGCTAGGCCAAAGTGC | 3182 |
| 2733 | TCCCTAGGTGAAGATGGAAA | 3183 |
| 2734 | GCCCCAACATAGTAATTCCT | 3184 |
| 2735 | CTCTGAGCTAGTTGATCTCG | 3185 |
| 2736 | GTAGTACTATACGCCATGGC | 3186 |
| 2737 | ACAGTGCTAAGAGGAGGACC | 3187 |
| 2738 | CTGAAGCTGAAGTAGAGCGC | 3188 |
| 2739 | CACGTCTAAAGAACACCCCC | 3189 |
| 2740 | GGTTACCCTACTTGGAGAGC | 3190 |
| 2741 | ATTCACTCTAAAGCTGGAAA | 3191 |
| 2742 | AGCCAGCTACATGTAGGGGT | 3192 |
| 2743 | GCTCTAAGTGCCATTGCCGT | 3193 |
| 2744 | GCGTACTTCTAACAGGTCAG | 3194 |
| 2745 | GCTGGGGATCTAGGGGCCGG | 3195 |
| 2746 | GCCGCTGTGGTGCACCACGC | 3196 |
| 2747 | CTCTAGGGCATGGGGTGGCT | 3197 |
| 2748 | GGAAGGTCTAAGAGATGGTA | 3198 |
| 2749 | GAGGACGACGACGTCACCAA | 3199 |

As a companion to the above Table 5, the following Table 6 indicates which indexed sgRNAs were identified per each base editor tested in Example 1:

TABLE 6

| | sgRNA (numerals correspond to the Index No. from the |
|---|---|
| Base Editor - amino acid | above Table - ranges are inclusive. Data at sgRNAs at |
| sequences are provided | indexes 1-2,695 are from SpCas9, while data at sgRNAs at |
| herein and indicated below | indexes 2,696-2,749 are from Cas9-NG) |
| ABE | 880-2498 |
| SEQ ID NO: 3210 | |
| ABE-CP1041 | 880-990, 998-1014, 1042-1313, 1749-2184, 2186-2695 |
| SEQ ID NO: 3211 | |
| AID-BE4 | 1-301 |
| SEQ ID NO: 3202 | |
| BE4 | 2-3, 6-12, 16-17, 19-27, 40-42, 44, 47-48, 52-53, 55-58, 62- |
| SEQ ID NO: 3200 | 65, 68, 70, 74-78, 80, 82-92, 94-98, 198, 200-204, 207, 210- |
| | 211, 213-219, 222-224, 226-229, 231-233, 235- |
| | 236, 238, 244, 247-248, 252-255, 257-258, 260, 263-270, 272- |
| | 275, 279, 281-287, 289-290, 293-294, 296, 298- |
| | 299, 301, 541, 543-626, 628-712, 722-723, 798-838, 840-848, 858-878 |
| BE4-CP1028 | 2-3, 5-9, 11-15, 17-27, 40, 42, 44, 47-50, 52-54, 56-58, 63, 65, 74- |
| SEQ ID NO: 3208 | 75, 77, 79-83, 85, 87-93, 96-98, 157, 162, 182, 263, 302, 305, 308, 313, |
| | 315, 324, 336, 338, 341, 343, 345, 403, 407-411, 413, 415- |
| | 416, 418-419, 421, 423-427, 429-440, 461-464, 467-468, 470- |
| | 471, 473, 508-514, 516-520, 522-524, 526-535, 537, 539- |
| | 540, 544, 586, 588-590, 592-605, 607, 621, 624, 632, 702- |
| | 703, 705-708, 710-712, 723, 799-801, 803-804, 807- |
| | 808, 810, 813-816, 818-828, 830-835, 837-838, 840-848, 858- |
| | 860, 864-873, 876-878 |
| CDA-BE4 | 4, 6-7, 9-13, 15-17, 20-24, 26, 31-32, 35, 40-41, 44, 47-50, 52- |
| SEQ ID NO: 3203 | 53, 55, 63-65, 68, 70-72, 75-81, 84-87, 89-94, 98, 100-101, 103- |
| | 104, 107, 109, 111, 113, 118-121, 124-127, 130-132, 136, 141- |
| | 144, 146-148, 151-160, 162, 164, 166-167,170, 172-173, 175- |
| | 180, 184, 195, 198, 200-204, 206-215, 218-219, 221-224, 226- |
| | 227, 230, 233-234, 237, 239, 243-244, 247, 251-257, 261- |
| | 267, 274, 281-284, 286-287, 289-290, 292, 295, 297- |
| | 302, 304, 411-412, 414, 417, 420, 422- |
| | 423, 425, 428, 431, 433, 435, 438, 442-445, 457, 463, 472, 477- |
| | 479, 485, 488, 491, 493- |
| | 494, 507, 510, 513, 515, 518, 521, 536, 538, 540, 542, 552, 561, |
| | 563-569, 573-582, 587-588, 591, 593-595, 598, 622- |
| | 623, 625, 627, 640, 667, 704, 712-721, 724-727, 734- |
| | 752, 755, 759, 761-768, 773-774, 776, 780, 785-786, 788- |
| | 789, 795-797, 800, 802, 805-806, 811-812, 814, 817- |
| | 818, 820, 829, 831, 833, 835, 839- |
| | 842, 849, 852, 854, 856, 861, 864, 874-875, 878-879 |
| eA3A-BE4 | 2-3, 6, 8-10, 13, 15-17, 20, 22-23, 25, 27-28, 32, 35, 42, |
| SEQ ID NO: 3205 | 45-47, 53, 55-56, 63-64, 74, 76, 80-81, 86-92, 96- |
| | 98, 111, 119, 121, 127, 151, 154, 156, 159- |
| | 160, 171, 178, 180, 184, 192, 198, 204-206, 210-211, 214, 216- |
| | 217, 220, 224, 228-229, 231-233, 235, 244, 247, 252- |
| | 253, 260, 263-268, 270, 272-274, 276, 279, 281-285, 287- |
| | 289, 293-294, 296, 298, 303-304, 306-312, 314, 316-317, 319- |
| | 323, 326-329, 331-337, 339, 343-345, 347-348, 352-362, 364- |
| | 372, 374-406, 410-411, 432-434, 438, 446-447, 449- |
| | 453, 456, 458, 460, 466, 468-469, 474-476, 481, 486, 489- |
| | 490, 492, 495-506, 521, 523, 525, 539, 543-551, 553-556, 558- |
| | 564, 569, 573, 575, 578-579, 581, 583-584, 588, 590, 593, 595- |
| | 596, 598-600, 602, 604, 607, 614-620, 622, 624, 626, 628- |
| | 630, 632-639, 641-647, 651, 657, 660, 662-663, 665-666, 668- |
| | 671, 673-674, 678, 686-689, 691-693, 695-700, 702-703, 707- |
| | 709, 711-712, 715, 723, 741, 800-806, 808, 811, 813-821, 823- |
| | 827, 829-830, 832-833, 835, 844, 846-849, 852, 858-860, 865- |
| | 866, 868-870, 872-874, 878, 2696-2737 |
| eA3A_T31AT44A | 2725-2726, 2738-2749 |
| evoAPOBEC1-BE4max | 1-4, 6-7, 9-11, 13, 15-18, 20, 22-27, 32, 35, 40-42, 44, 47-49, |
| SEQ ID NO: 3204 | 51-53, 55-56, 58, 61-63, 68, 70-72, 74, 76-82, 84-92, 94- |
| | 98, 100, 104, 108, 111, 116, 121, 125-126, 131, 136, 141-143, |
| | 146-148, 150-151, 153, 155-160, 162, 170, 172, 175, 178-180, |
| | 183-184, 190, 195, 198, 200-201, 203-204, 206, 210- |
| | 212, 214, 217, 220-221, 223-227, 229, 231-233, 235- |
| | 239, 244, 247, 249, 252-258, 263-270, 272-274, 276, 278- |
| | 279, 281-284, 286-290, 293-294, 296, 298, 300- |
| | 301, 304, 318, 321, 324-325, 330-333, 338, 340, 342, 346, |
| | 349-351, 358, 363, 373, 379-380, 385- |
| | 389, 411, 423, 425, 427, 431, 433, 438, 441, 445, 448, 454- |
| | 455, 459, 463, 465, 472, 476, 480, 482-484, 487, 491, 493- |
| | 494, 503, 510, 514, 517, 521, 535, 540, 542, 544-545, 551- |
| | 555, 558-564, 567-568, 573-576, 579-582, 588-589, 593, 595- |
| | 596, 598, 600, 603, 605, 610, 612-617, 620, 622, 625- |
| | 626, 628, 630-631, 635-641, 644, 651, 653-654, 656, 676, 678- |
| | 679, 682, 688, 694, 704, 711, 713-715, 717, 720-723, 728- |
| | 734, 742-743, 745, 747, 750, 752-754, 756- |
| | 758, 760, 762, 766, 769-773, 775, 777-779, 781-784, 787, 790- |
| | 794, 798, 800, 803, 805-806, 809, 811-812, 814, 818-819, 824- |
| | 825, 827, 829, 831, 833, 835, 838-839, 841-842, 847, 850- |
| | 855, 857-859, 861, 864, 870-873, 875, 878-879 |

Accordingly, the present disclosure provides a guide RNA for use in a base editing system for introducing a target change into a target DNA sequence identified by the BE-Hive method disclosed herein.

In some embodiments, the guide RNA comprises a protospacer selected from the group consisting of SEQ ID Nos: 451-3199 of Table 5. The guide RNA of Table 5 are those where at least one base editor demonstrated at least 50% correction precision to the wild-type genotype among edited reads in accordance with Example 1. The base editors used in Example 1 can be ABE (SEQ ID NO: 3210), ABE-CP1041 (SEQ ID NO: 3211), AID-BE4 (SEQ ID NO: 3202), BE4 (SEQ ID NO: 3200), BE4-CP1028 (SEQ ID NO: 3208), CDA-BE4 (SEQ ID NO: 3203), eA3A-BE4 (SEQ ID NO: 3205), eA3A_T31AT44A, or evoAPOBEC1-BE4max (SEQ ID NO: 3204).

In some embodiments, the base editing system comprises an ABE of SEQ ID NO: 3210 and said guide RNA comprises a protospacer identified as any of the sequences of Index Nos. 880-2498 of Table 5.

In other embodiments, the base editing system comprises an ABE-CP1041 of SEQ ID NO: 3211, and said guide RNA comprises a protospacer identified as any of the sequences of Index Nos. 880-990, 998-1014, 1042-1313, 1749-2184, 2186-2695 of Table 5.

In still other embodiments, the base editing system comprises an AID-BE4 of SEQ ID NO: 3202, and said guide RNA comprises a protospacer identified as any of the sequences of Index Nos. 1-301 of Table 5.

In other embodiments, the base editing system comprises an BE4 of SEQ ID NO: 3200, and said guide RNA comprises a protospacer identified as any of the sequences of Index Nos. 2-3, 6-12, 16-17, 19-27, 40-42, 44, 47-48, 52-53, 55-58, 62-65, 68, 70, 74-78, 80, 82-92, 94-98, 198, 200-204, 207, 210-211, 213-219, 222-224, 226-229, 231-233, 235-236, 238, 244, 247-248, 252-255, 257-258, 260, 263-270, 272-275, 279, 281-287, 289-290, 293-294, 296, 298-299, 301, 541, 543-626, 628-712, 722-723, 798-838, 840-848, 858-878 of Table 5.

In still other embodiments, the base editing system comprises an BE4-CP1028 of SEQ ID NO: 3208, and said guide RNA comprises a protospacer identified as any of the sequences of Index Nos. 2-3, 5-9, 11-15, 17-27, 40, 42, 44, 47-50, 52-54, 56-58, 63, 65, 74-75, 77, 79-83, 85, 87-93, 96-98, 157, 162, 182, 263, 302, 305, 308, 313, 315, 324, 336, 338, 341, 343, 345, 403, 407-411, 413, 415-416, 418-419, 421, 423-427, 429-440, 461-464, 467-468, 470-471, 473, 508-514, 516-520, 522-524, 526-535, 537, 539-540, 544, 586, 588-590, 592-605, 607, 621, 624, 632, 702-703, 705-708, 710-712, 723, 799-801, 803-804, 807-808, 810, 813-816, 818-828, 830-835, 837-838, 840-848, 858-860, 864-873, 876-878 of Table 5.

In yet other embodiments, the base editing system comprises an CDA-BE4 of SEQ ID NO: 3203, and said guide RNA comprises a protospacer identified as any of the sequences of Index Nos. 4, 6-7, 9-13, 15-17, 20-24, 26, 31-32, 35, 40-41, 44, 47-50, 52-53, 55, 63-65, 68, 70-72, 75-81, 84-87, 89-94, 98, 100-101, 103-104, 107, 109, 111, 113, 118-121, 124-127, 130-132, 136, 141-144, 146-148, 151-160, 162, 164, 166-167, 170, 172-173, 175-180, 184, 195, 198, 200-204, 206-215, 218-219, 221-224, 226-227, 230, 233-234, 237, 239, 243-244, 247, 251-257, 261-267, 274, 281-284, 286-287, 289-290, 292, 295, 297-302, 304, 411-412, 414, 417, 420, 422-423, 425, 428, 431, 433, 435, 438, 442-445, 457, 463, 472, 477-479, 485, 488, 491, 493-494, 507, 510, 513, 515, 518, 521, 536, 538, 540, 542, 552, 561, 563-569, 573-582, 587-588, 591, 593-595, 598, 622-623, 625, 627, 640, 667, 704, 712-721, 724-727, 734-752, 755, 759, 761-768, 773-774, 776, 780, 785-786, 788-789, 795-797, 800, 802, 805-806, 811-812, 814, 817-818, 820, 829, 831, 833, 835, 839-842, 849, 852, 854, 856, 861, 864, 874-875, 878-879 of Table 5.

In still other embodiments, the base editing system comprises an eA3A-BE4 of SEQ ID NO: 3204, and said guide RNA comprises a protospacer identified as any of the sequences of Index Nos. 2-3, 6, 8-10, 13, 15-17, 20, 22-23, 25, 27-28, 32, 35, 42, 45-47, 53, 55-56, 63-64, 74, 76, 80-81, 86-92, 96-98, 111, 119, 121, 127, 151, 154, 156, 159-160, 171, 178, 180, 184, 192, 198, 204-206, 210-211, 214, 216-217, 220, 224, 228-229, 231-233, 235, 244, 247, 252-253, 260, 263-268, 270, 272-274, 276, 279, 281-285, 287-289, 293-294, 296, 298, 303-304, 306-312, 314, 316-317, 319-323, 326-329, 331-337, 339, 343-345, 347-348, 352-362, 364-372, 374-406, 410-411, 432-434, 438, 446-447, 449-453, 456, 458, 460, 466, 468-469, 474-476, 481, 486, 489-490, 492, 495-506, 521, 523, 525, 539, 543-551, 553-556, 558-564, 569, 573, 575, 578-579, 581, 583-584, 588, 590, 593, 595-596, 598-600, 602, 604, 607, 614-620, 622, 624, 626, 628-630, 632-639, 641-647, 651, 657, 660, 662-663, 665-666, 668-671, 673-674, 678, 686-689, 691-693, 695-700, 702-703, 707-709, 711-712, 715, 723, 741, 800-806, 808, 811, 813-821, 823-827, 829-830, 832-833, 835, 844, 846-849, 852, 858-860, 865-866, 868-870, 872-874, 878, 2696-2737 of Table 5.

In still other embodiments, the base editing system comprises an eA3A_T31AT44A of SEQ ID NO: 3206, and said guide RNA comprises a protospacer identified as any of the sequences of Index Nos. 2725-2726 and 2738-2749 of Table 5.

In still other embodiments, the base editing system comprises an evoAPOBEC1-BE4max of SEQ ID NO: 3204, and said guide RNA comprises a protospacer identified as any of the sequences of Index Nos. 1-4, 6-7, 9-11, 13, 15-18, 20, 22-27, 32, 35, 40-42, 44, 47-49, 51-53, 55-56, 58, 61-63, 68, 70-72, 74, 76-82, 84-92, 94-98, 100, 104, 108, 111, 116, 121, 125-126, 131, 136, 141-143, 146-148, 150-151, 153, 155-160, 162, 170, 172, 175, 178-180, 183-184, 190, 195, 198, 200-201, 203-204, 206, 210-212, 214, 217, 220-221, 223-227, 229, 231-233, 235-239, 244, 247, 249, 252-258, 263-270, 272-274, 276, 278-279, 281-284, 286-290, 293-294, 296, 298, 300-301, 304, 318, 321, 324-325, 330-333, 338, 340, 342, 346, 349-351, 358, 363, 373, 379-380, 385-389, 411, 423, 425, 427, 431, 433, 438, 441, 445, 448, 454-455, 459, 463, 465, 472, 476, 480, 482-484, 487, 491, 493-494, 503, 510, 514, 517, 521, 535, 540, 542, 544-545, 551-555, 558-564, 567-568, 573-576, 579-582, 588-589, 593, 595-596, 598, 600, 603, 605, 610, 612-617, 620, 622, 625-626, 628, 630-631, 635-641, 644, 651, 653-654, 656, 676, 678-679, 682, 688, 694, 704, 711, 713-715, 717, 720-723, 728-734, 742-743, 745, 747, 750, 752-754, 756-758, 760, 762, 766, 769-773, 775, 777-779, 781-784, 787, 790-794, 798, 800, 803, 805-806, 809, 811-812, 814, 818-819, 824-825, 827, 829, 831, 833, 835, 838-839, 841-842, 847, 850-855, 857-859, 861, 864, 870-873, 875, 878-879 of Table 5.

### General Considerations in Guide RNA Design

In general, a guide sequence is any polynucleotide sequence having sufficient complementarity with a target polynucleotide sequence to hybridize with the target sequence and direct sequence-specific binding of a napDNAbp (e.g., a Cas9, Cas9 homolog, or Cas9 variant) to the target sequence, such as a sequence within an SMN2 gene that comprises a C840T point mutation. In some embodiments, the degree of complementarity between a guide sequence and its corresponding target sequence (e.g., SMN2), when optimally aligned using a suitable alignment algorithm, is about or more than about 50%, 60%, 75%, 80%, 85%, 90%, 95%, 97.5%, 99%, or more. Optimal alignment may be determined with the use of any suitable algorithm for aligning sequences, non-limiting example of which include the Smith-Waterman algorithm, the Needleman-Wunsch algorithm, algorithms based on the Burrows-Wheeler Transform (e.g. the Burrows Wheeler Aligner), ClustalW, Clustal X, BLAT, Novoalign (Novocraft Technologies, ELAND (Illumina, San Diego, Calif.), SOAP (available at soap.genomics.org.cn), and Maq (available at maq.sourceforge.net). In some embodiments, a guide sequence is about or more than about 5, 10, 11, 12, 13, 14, 15, 16, 17, 18, 19, 20, 21, 22, 23, 24, 25, 26, 27, 28, 29, 30, 31, 32, 33, 34, 35, 40, 45, 50, 75, or more nucleotides in length.

In some embodiments, a guide sequence is less than about 75, 50, 45, 40, 35, 30, 25, 20, 15, 12, or fewer nucleotides in length. The ability of a guide sequence to direct sequence-specific binding of a base editor to a target sequence may be assessed by any suitable assay. For example, the components of a base editor, including the guide sequence to be tested, may be provided to a host cell having the corresponding target sequence (e.g., a HGADFN 167 or HGADFN 188 cell line), such as by transfection with vectors encoding the components of a base editor disclosed herein, followed by an assessment of preferential cleavage within the target sequence, such as by Surveyor assay as described herein. Similarly, cleavage of a target polynucleotide sequence may be evaluated in a test tube by providing the target sequence, components of a base editor, including the guide sequence to be tested and a control guide sequence different from the test guide sequence, and comparing binding or rate of cleavage at the target sequence between the test and control guide sequence reactions. Other assays are possible, and will occur to those skilled in the art.

In some embodiments, a guide sequence is selected to reduce the degree of secondary structure within the guide sequence. Secondary structure may be determined by any suitable polynucleotide folding algorithm. Some programs are based on calculating the minimal Gibbs free energy. An example of one such algorithm is mFold, as described by Zuker and Stiegler (Nucleic Acids Res. 9 (1981), 133-148). Another example folding algorithm is the online webserver RNAfold, developed at Institute for Theoretical Chemistry at the University of Vienna, using the centroid structure prediction algorithm (see e.g. A. R. Gruber et al., 2008, Cell 106(1): 23-24; and P A Carr and G M Church, 2009, Nature Biotechnology 27(12): 1151-62). Further algorithms may be found in U.S. application Ser. No. 61/836,080; Broad Reference BI-2013/004A); incorporated herein by reference.

In general, a tracr mate sequence includes any sequence that has sufficient complementarity with a tracr sequence to promote one or more of: (1) excision of a guide sequence flanked by tracr mate sequences in a cell containing the corresponding tracr sequence; and (2) formation of a complex at a target sequence, wherein the complex comprises the tracr mate sequence hybridized to the tracr sequence. In general, degree of complementarity is with reference to the optimal alignment of the tracr mate sequence and tracr sequence, along the length of the shorter of the two sequences. Optimal alignment may be determined by any suitable alignment algorithm, and may further account for secondary structures, such as self-complementarity within either the tracr sequence or tracr mate sequence. In some embodiments, the degree of complementarity between the tracr sequence and tracr mate sequence along the length of the shorter of the two when optimally aligned is about or more than about 25%, 30%, 40%, 50%, 60%, 70%, 80%, 90%, 95%, 97.5%, 99%, or higher. In some embodiments, the tracr sequence is about or more than about 5, 6, 7, 8, 9, 10, 11, 12, 13, 14, 15, 16, 17, 18, 19, 20, 25, 30, 40, 50, or more nucleotides in length. In some embodiments, the tracr sequence and tracr mate sequence are contained within a single transcript, such that hybridization between the two produces a transcript having a secondary structure, such as a hairpin. Preferred loop forming sequences for use in hairpin structures are four nucleotides in length, and most preferably have the sequence GAAA. However, longer or shorter loop sequences may be used, as may alternative sequences. The sequences preferably include a nucleotide triplet (for example, AAA), and an additional nucleotide (for example C or G). Examples of loop forming sequences include CAAA and AAAG. In an embodiment of the invention, the transcript or transcribed polynucleotide sequence has at least two or more hairpins. In preferred embodiments, the transcript has two, three, four or five hairpins. In a further embodiment of the invention, the transcript has at most five hairpins. In some embodiments, the single transcript further includes a transcription termination sequence; preferably this is a polyT sequence, for example six T nucleotides. Further non-limiting examples of single polynucleotides comprising a guide sequence, a tracr mate sequence, and a tracr sequence are as follows (listed 5′ to 3′), where “N” represents a base of a guide sequence, the first block of lower case letters represent the tracr mate sequence, and the second block of lower case letters represent the tracr sequence, and the final poly-T sequence represents the transcription terminator:

(SEQ ID NO: 297)
(1) NNNNNNNNgtttttgtactctcaagatttaGAAAtaaatcttgcag
aagctacaaagataaggcttcatgccgaaatcaacaccctgtcattttat
ggcagggtgttttcgttatttaaTTTTTT;
(SEQ ID NO: 298)
(2) NNNNNNNNNNNNNNNNNNgtttttgtactctcaGAAAtgcagaagc
tacaaagataaggcttcatgccgaaatcaacaccctgtcattttatggca
gggtgttttcgttatttaaTTTTTT;
(SEQ ID NO: 299)
(3) NNNNNNNNNNNNNNNNNNNNgtttttgtactctcaGAAAtgcagaa
gctacaaagataaggcttcatgccgaaatca acaccctgtcattttatg
gcagggtgtTTTTT;
(SEQ ID NO: 300)
(4) NNNNNNNNNNNNNNNNNNNNgttttagagctaGAAAtagcaagtta
aaataaggctagtccgttatcaacttgaaaa agtggcaccgagtcggtg
cTTTTTT;
(SEQ ID NO: 301)
(5) NNNNNNNNNNNNNNNNNNNNgttttagagctaGAAATAGcaagtta
aaataaggctagtccgttatcaacttgaa aaagtgTTTTTTT;
and
(SEQ ID NO: 302)
(6) NNNNNNNNNNNNNNNNNNNNgttttagagctagAAATAGcaagtta
aaataaggctagtccgttatcaTTTTT TTT.

The disclosure also relates to guide RNA sequences that are variants of any of the herein disclosed guide RNA sequences or target sequences (including SEQ ID NOs.: 250-302), wherein the variants include guide RNA sequences or target sequences having a deletion of 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 nucleotides from any of the guide RNA or target sequence disclosed herein (e.g., SEQ ID NOs.: 250-302). In other embodiments, the variants also include guide RNA sequences or target sequences having at least 70%, 71%, 72%, 73%, 74%, 75%, 76%, 77%, 78%7, 79%, 80%, 81%, 82%, 83%, 84%, 85%, 86%, 87%, 88%, 89%, 90%, 91%, 92%, 93%, 94%, 95%, 96%, 97%, 98%, 99%, or up to 99.9% sequence identity with a guide RNA or target sequence disclosed herein (e.g., SEQ ID NOs.: 250-302).

In some embodiments, sequences (1) to (3) are used in combination with Cas9 from S. thermophilus CRISPR. In some embodiments, sequences (4) to (6) are used in combination with Cas9 from S. pyogenes. In some embodiments, the tracr sequence is a separate transcript from a transcript comprising the tracr mate sequence.

It will be apparent to those of skill in the art that in order to target any of the fusion proteins comprising a Cas9 domain and an adenosine deaminase, as disclosed herein, to a target site, e.g., a site comprising a C840T point mutation in SMN2 to be edited, it is typically necessary to co-express the fusion protein together with a guide RNA, e.g., an sgRNA. As explained in more detail elsewhere herein, a guide RNA typically comprises a tracrRNA framework allowing for Cas9 binding, and a guide sequence, which confers sequence specificity to the Cas9:nucleic acid editing enzyme/domain fusion protein.

In some embodiments, the guide RNA comprises a structure 5′-[guide sequence]-[Cas9-binding sequence]-3′, where the Cas9 binding sequence comprises a nucleic acid sequence that is at least 70%, 75%, 80%, 85%, 90%, 95%, 96%, 97%, 98%, 99%, or 99.5% identical to SEQ ID NO: 306-323, and which are effective to targeting the C840T point mutation in SMN2. In other embodiments, the guide RNA comprises a structure 5′-[guide sequence]-[Cas9-binding sequence]-3′, where the Cas9 binding sequence comprises a nucleic acid sequence that is at least 70%, 75%, 80%, 85%, 90%, 95%, 96%, 97%, 98%, 99%, or 99.5% identical to SEQ ID NO: 324-329, and which are effective to targeting a stop codon in exon 8 of SMN2. In yet other embodiments, the guide RNA comprises a structure 5′-[guide sequence]-[Cas9-binding sequence]-3′, where the Cas9 binding sequence comprises a nucleic acid sequence that is at least 70%, 75%, 80%, 85%, 90%, 95%, 96%, 97%, 98%, 99%, or 99.5% identical to SEQ ID NO: 330, and which are effective to targeting the S270 amino acid in exon 6 of SMN2. In some embodiments, the guide RNA comprises a structure 5′-[guide sequence]-[Cas9-binding sequence]-3′, where the Cas9 binding sequence comprises a nucleic acid sequence SEQ ID NO: 303, SEQ ID NO: 304, or SEQ ID NO: 303 or 304 absent the poly-U terminator sequence at the 3′ end.

(SEQ ID NO: 303)
5′GUUUUAGAGCUAGAAAUAGCAAGUUAAAAUAAAGGCUAGUCCGUUAU
CAACUUGAAAAAGUGGCACCGAGUCGGUGCUUUUU-3′
(SEQ ID NO: 304)
5′GUUUUAGAGCUAGAAAUAGCAAGUUAAAAUAAGGCUAGUCCGUUAUC
AACUUGAAAAAGUGGCACCGAGUCGGUGCUUUUUUU-3′

In some embodiments, the guide RNA comprises a nucleic acid sequence that is at least 70%, 75%, 80%, 85%, 90%, 95%, 96%, 97%, 98%, 99%, or 99.5% identical to SEQ ID NO: 305, or SEQ ID NO: 305 absent the poly-U terminator sequence at the 3′ end. In some embodiments, the guide RNA comprises the nucleic acid sequence SEQ ID NO: 305, or SEQ ID NO: 305 absent the poly-U terminator sequence at the 3′ end.

In some embodiments, the guide RNA comprises the nucleic acid sequence

(SEQ ID NO: 305)
5′GGUCCACCCACCUGGGCUCCGUUUUAGAGCUAGAAAUAGCAAGUUAA
AAUAAGGCUAGUCCGUUAUCAACUUGAAAAAGUGGCACCGAGUCGGUGC
UUUUUUU-3′.

The disclosure also provides guide sequences that are truncated variants of any of the guide sequences provided herein (e.g., SEQ ID NOs: 306-330). In some embodiments, the guide sequence comprises the amino acid sequence of any one of SEQ ID NOs: 306-330, absent the first 1, 2, 3, 4, 5, 6, 7, 8, 9, or 10 nucleic acid residues from the 5′ end. It should be appreciated that any of the 5′ truncated guide sequences provided herein may further comprise a G residue at the 5′ end. In some embodiments, the guide sequence comprises the amino acid sequence of any one of SEQ ID NOs: 306-330, absent the first 1, 2, 3, 4, 5, 6, 7, 8, 9, 10, 11, or 12 nucleic acid residues from the 3′ end.

The disclosure also provides guide sequences that are longer variants of any of the guide sequences provided herein (e.g., SEQ ID NOs: 306-330). In some embodiments, the guide sequence comprises one additional residue that is 5′-U-3′ at the 3′ end of any one of SEQ ID NOs: 306-330. In some embodiments, the guide sequence comprises two additional residues that are 5′-UG-3′ at the 3′ end of any one of SEQ ID NOs: 306-330. In some embodiments, the guide sequence comprises three additional residues that are 5′-UGA-3′ at the 3′ end of any one of SEQ ID NOs: 306-330. In some embodiments, the guide sequence comprises four additional residues that are 5′-UGAG-3′ at the 3′ end of any one of SEQ ID NOs: 306-330. In some embodiments, the guide sequence comprises five additional residues that are 5′-UGAGC-3′ at the 3′ end of any one of SEQ ID NOs: 306-330. In some embodiments, the guide sequence comprises six additional residues that are 5′-UGAGCC-3′ at the 3′ end of any one of SEQ ID NOs: 306-330. In some embodiments, the guide sequence comprises seven additional residues that are 5′-UGAGCCG-3′ at the 3′ end of any one of SEQ ID NOs: 306-330. In some embodiments, the guide sequence comprises eight additional residues that are 5′-UGAGCCGC-3′ at the 3′ end of any one of SEQ ID NOs: 306-330. In some embodiments, the guide sequence comprises nine additional residues that are 5′-UGAGCCGCU-3′ at the 3′ end of any one of SEQ ID NOs: 306-330. In some embodiments, the guide sequence comprises ten additional residues that are 5′-UGAGCCGCUG-3′ (SEQ ID NO: 400) at the 3′ end of any one of SEQ ID NOs: 306-330. In some embodiments, the guide sequence comprises eleven additional residues that are 5′-UGAGCCGCUGG-3′ (SEQ ID NO: 401) at the 3′ end of any one of SEQ ID NOs: 306-330.

VII. Fusion Protein/2RNA Complexes

Some aspects of this disclosure provide complexes comprising any of the fusion proteins (e.g., base editor) provided herein, for example any of the adenosine base editors provided herein, and a guide nucleic acid bound to napDNAbp of the fusion protein. In some embodiments, the guide nucleic acid is any one of the guide RNAs provided herein. In some embodiments, the disclosure provides any of the fusion proteins (e.g., adenosine base editors) provided herein bound to any of the guide RNAs provided herein. In some embodiments, the napDNAbp of the fusion protein (e.g., adenosine base editor) is a Cas9 domain (e.g., a dCas9, a nuclease active Cas9, or a Cas9 nickase), which is bound to a guide RNA. In some embodiments, the complexes provided herein are configured to generate a mutation in a nucleic acid, for example to correct a point mutation in a gene (e.g., SMN2) to modulate expression of one or more proteins (e.g., SMN).

In some embodiments, the guide RNA comprises a guide sequence that comprises at least 8, 9, 10, 11, 12, 13, 14, 15, 16, 17, 18, 19, 20, 21, 22, 23, 24, 25, 26, 27, 28, 29, or 30 contiguous nucleic acids that are 100% complementary to a target sequence, for example a target DNA sequence (e.g., a target DNA sequence of any one of SEQ ID NOs: 253-296 and 398-399). In some embodiments, the guide RNA comprises a guide sequence that comprises at least 8, 9, 10, 11, 12, 13, 14, 15, 16, 17, 18, 19, 20, 21, 22, 23, 24, 25, 26, 27, 28, 29, or 30 contiguous nucleic acids that are 100% complementary to a DNA sequence in a SMN2 gene (e.g., a target DNA sequence of any one of SEQ ID NOs: 253-296 and 398-399), for example a region of a human SMN2 gene.

In some embodiments, any of the complexes provided herein comprise a gRNA having a guide sequence that comprises at least 8, 9, 10, 11, 12, 13, 14, 15, 16, 17, 18, 19, 20, 21, 22, 23, 24, 25, 26, 27, 28, 29, or 30 contiguous nucleic acids that are 100% complementary to any one of the nucleic acid sequences provided herein. It should be appreciated that the guide sequence of the gRNA may comprise one or more nucleotides that are not complementary to a target sequence. In some embodiments, the guide sequence of the gRNA is at the 5′ end of the gRNA. In some embodiments, the guide sequence of the gRNA further comprises a G at the 5′ end of the gRNA. In some embodiments, the G at the 5′ end of the gRNA is not complementary with the target sequence. In some embodiments, the guide sequence of the gRNA comprises 1, 2, 3, 4, 5, 6, 7, or 8 nucleotides that are not complementary to a target sequence (e.g., any of the target sequences provided herein (e.g., SEQ ID NOs: 297-305, 306-362, 400-401, and 405-406)). In some embodiments, the gRNA comprises the sequence of SEQ ID NO: 297, or the sequence of any one of SEQ ID NOs: 297-305, 306-362, 400-401, and 405-406, where the nucleotide target is indicated in bold. It should be appreciated that the T's indicated in any of the gRNA sequences of SEQ ID NOs: 297-305, 306-362, 400-401, and 405-406 are uricils (Us) in the RNA sequence. Accordingly, in some embodiments, the gRNA comprises the sequence 5′-AUUUUGUCUAAAACCCUGUA-3′ (SEQ ID NO: 312).

A complex comprising a base editor and a guide RNA selected from the method of claim 1 or a guide RNA of any one of claims 52-64.

The complex of claim 65, wherein the base editor comprises a napDNAbp.

The complex of claim 66, wherein the napDNAbp is a Cas9 or variant thereof.

The complex of claim 66, wherein the napDNAbp is a wildtype SpCas9 comprising an amino acid sequence of SEQ ID NO: 5, or an amino acid sequence having at least 80%, 85%, 90%, 95%, or 99% sequence identity with SEQ ID NO: 5.

The complex of claim 66, wherein the napDNAbp is a wildtype SpCas9 comprising an amino acid sequence of SEQ ID NOs: 5, 8, 10, 12, and 407 or an amino acid sequence having at least 80%, 85%, 90%, 95%, or 99% sequence identity with any one of SEQ ID NOs: 5, 8, 10, 12, or 407.

The complex of claim 66, wherein the napDNAbp is a SpCas9 ortholog or homolog comprising an amino acid sequence of SEQ ID Nos: 13-26, 44-63, or 74-77, or an amino acid sequence having at least 80%, 85%, 90%, 95%, or 99% sequence identity with any one of SEQ ID NOs: 13-26, 44-63, or 74-77.

The complex of claim 66, wherein the napDNAbp is a dead Cas9 comprising an amino acid sequence of SEQ ID Nos: 27-28, or an amino acid sequence having at least 80%, 85%, 90%, 95%, or 99% sequence identity with any one of SEQ ID NOs: 27-28.

The complex of claim 66, wherein the napDNAbp is a nickase Cas9 comprising an amino acid sequence of SEQ ID Nos: 29-44, or an amino acid sequence having at least 80%, 85%, 90%, 95%, or 99% sequence identity with any one of SEQ ID NOs: 29-44.

The complex of claim 66, wherein the napDNAbp is a circular permutant variant of Cas9 comprising an amino acid sequence of SEQ ID Nos: 64-73, or an amino acid sequence having at least 80%, 85%, 90%, 95%, or 99% sequence identity with any one of SEQ ID NOs: 64-73.

The complex of claim 65, wherein the base editor comprises an adenine deaminase.

The complex of claim 65, wherein the base editor comprises a cytidine deaminase.

The complex of claim 74, wherein the adenine deaminase comprises an amino acid sequence of any one of SEQ ID NOs: 78-91, 403, or 462, or an amino acid sequence having at least 80%, 85%, 90%, 95%, or 99% sequence identity with any one of SEQ ID NOs: 78-91, 403, or 462.

The complex of claim 75, wherein the cytidine deaminase comprises an amino acid sequence of any one of SEQ ID NOs: 92-134, or an amino acid sequence having at least 80%, 85%, 90%, 95%, or 99% sequence identity with any one of SEQ ID NOs: 92-134.

The complex of claim 65, wherein the base editor comprises one or more linkers having an amino acid sequence comprising any one of SEQ ID NOs.: 135-151, or an amino acid sequence having at least 80%, 85%, 90%, 95%, or 99% sequence identity with any one of SEQ ID NOs: 135-151.

The complex of claim 65, wherein the base editor comprises one or more NLS having an amino acid sequence comprising any one of SEQ ID NOs.: 152-162, or an amino acid sequence having at least 80%, 85%, 90%, 95%, or 99% sequence identity with any one of SEQ ID NOs: 152-162.

The complex of claim 65, wherein the base editor comprises one or more UGI having an amino acid sequence comprising SEQ ID NO.: 163, or an amino acid sequence having at least 80%, 85%, 90%, 95%, or 99% sequence identity with SEQ ID NO:163.

The complex of claim 65, wherein the base editor is an adenosine base editor comprising an amino acid sequence of any one of SEQ ID NOs: 174-221 or 463-476, or an amino acid sequence having at least 80%, 85%, 90%, 95%, or 99% sequence identity with any one of SEQ ID NOs: 174-221 or 463-476.

The complex of claim 65, wherein the base editor is a cytidine base editor comprising an amino acid sequence of any one of SEQ ID NOs: 223-248, or an amino acid sequence having at least 80%, 85%, 90%, 95%, or 99% sequence identity with any one of SEQ ID NOs: 223-248.

The complex of claim 65, wherein the base editor is ABE (SEQ ID NO: 3210), ABE-CP1041 (SEQ ID NO: 3211), AID-BE4 (SEQ ID NO: 3202), BE4 (SEQ ID NO: 3200), BE4-CP1028 (SEQ ID NO: 3208), CDA-BE4 (SEQ ID NO: 3203), eA3A-BE4 (SEQ ID NO: 3205), eA3A_T31AT44A (SEQ ID NO: 3206), or evoAPOBEC1-BE4max (SEQ ID NO: 3204), or an amino acid sequence having at least 80%, 85%, 90%, 95%, or 99% sequence identity with any one of SEQ ID NOs: 3210, 3211, 3202, 3200, 3208, 3203, 3205, 3206, or 3204.

The complex of claim 65, wherein the guide RNA comprises a spacer corresponding to any one of the protospacers of SEQ ID Nos: 451-3199.

The complex of claim 65, wherein the base editing system comprises an ABE of SEQ ID NO: 3210 and said guide RNA comprises a spacer corresponding to any one of the protospacers identified as a sequence of Index Nos. 880-2498 of Table 6.

The complex of claim 65, wherein the base editing system comprises an ABE-CP1041 of SEQ ID NO: 3211, said guide RNA comprises a spacer corresponding to any one of the protospacers identified as a sequence of Index Nos. 880-990, 998-1014, 1042-1313, 1749-2184, 2186-2695 of Table 6.

The complex of claim 65, wherein the base editing system comprises an AID-BE4 of SEQ ID NO: 3202, said guide RNA comprises a spacer corresponding to any one of the protospacers identified as a sequence of Index Nos. 1-301 of Table 6.

The complex of claim 65, wherein the base editing system comprises an BE4 of SEQ ID NO: 3200, said guide RNA comprises a spacer corresponding to any one of the protospacers identified as a sequence of Index Nos. 2-3, 6-12, 16-17, 19-27, 40-42, 44, 47-48, 52-53, 55-58, 62-65, 68, 70, 74-78, 80, 82-92, 94-98, 198, 200-204, 207, 210-211, 213-219, 222-224, 226-229, 231-233, 235-236, 238, 244, 247-248, 252-255, 257-258, 260, 263-270, 272-275, 279, 281-287, 289-290, 293-294, 296, 298-299, 301, 541, 543-626, 628-712, 722-723, 798-838, 840-848, 858-878 of Table 6.

The complex of claim 65, wherein the base editing system comprises an BE4-CP1028 of SEQ ID NO: 3208, said guide RNA comprises a spacer corresponding to any one of the protospacers identified as a sequence of Index Nos. 2-3, 5-9, 11-15, 17-27, 40, 42, 44, 47-50, 52-54, 56-58, 63, 65, 74-75, 77, 79-83, 85, 87-93, 96-98, 157, 162, 182, 263, 302, 305, 308, 313, 315, 324, 336, 338, 341, 343, 345, 403, 407-411, 413, 415-416, 418-419, 421, 423-427, 429-440, 461-464, 467-468, 470-471, 473, 508-514, 516-520, 522-524, 526-535, 537, 539-540, 544, 586, 588-590, 592-605, 607, 621, 624, 632, 702-703, 705-708, 710-712, 723, 799-801, 803-804, 807-808, 810, 813-816, 818-828, 830-835, 837-838, 840-848, 858-860, 864-873, 876-878 of Table 6.

The complex of claim 65, wherein the base editing system comprises an CDA-BE4 of SEQ ID NO: 3203, said guide RNA comprises a spacer corresponding to any one of the protospacers identified as a sequence of Index Nos. 4, 6-7, 9-13, 15-17, 20-24, 26, 31-32, 35, 40-41, 44, 47-50, 52-53, 55, 63-65, 68, 70-72, 75-81, 84-87, 89-94, 98, 100-101, 103-104, 107, 109, 111, 113, 118-121, 124-127, 130-132, 136, 141-144, 146-148, 151-160, 162, 164, 166-167, 170, 172-173, 175-180, 184, 195, 198, 200-204, 206-215, 218-219, 221-224, 226-227, 230, 233-234, 237, 239, 243-244, 247, 251-257, 261-267, 274, 281-284, 286-287, 289-290, 292, 295, 297-302, 304, 411-412, 414, 417, 420, 422-423, 425, 428, 431, 433, 435, 438, 442-445, 457, 463, 472, 477-479, 485, 488, 491, 493-494, 507, 510, 513, 515, 518, 521, 536, 538, 540, 542, 552, 561, 563-569, 573-582, 587-588, 591, 593-595, 598, 622-623, 625, 627, 640, 667, 704, 712-721, 724-727, 734-752, 755, 759, 761-768, 773-774, 776, 780, 785-786, 788-789, 795-797, 800, 802, 805-806, 811-812, 814, 817-818, 820, 829, 831, 833, 835, 839-842, 849, 852, 854, 856, 861, 864, 874-875, 878-879 of Table 6.

The complex of claim 65, wherein the base editing system comprises an eA3A-BE4 of SEQ ID NO: 3204, said guide RNA comprises a spacer corresponding to any one of the protospacers identified as a sequence of Index Nos. 2-3, 6, 8-10, 13, 15-17, 20, 22-23, 25, 27-28, 32, 35, 42, 45-47, 53, 55-56, 63-64, 74, 76, 80-81, 86-92, 96-98, 111, 119, 121, 127, 151, 154, 156, 159-160, 171, 178, 180, 184, 192, 198, 204-206, 210-211, 214, 216-217, 220, 224, 228-229, 231-233, 235, 244, 247, 252-253, 260, 263-268, 270, 272-274, 276, 279, 281-285, 287-289, 293-294, 296, 298, 303-304, 306-312, 314, 316-317, 319-323, 326-329, 331-337, 339, 343-345, 347-348, 352-362, 364-372, 374-406, 410-411, 432-434, 438, 446-447, 449-453, 456, 458, 460, 466, 468-469, 474-476, 481, 486, 489-490, 492, 495-506, 521, 523, 525, 539, 543-551, 553-556, 558-564, 569, 573, 575, 578-579, 581, 583-584, 588, 590, 593, 595-596, 598-600, 602, 604, 607, 614-620, 622, 624, 626, 628-630, 632-639, 641-647, 651, 657, 660, 662-663, 665-666, 668-671, 673-674, 678, 686-689, 691-693, 695-700, 702-703, 707-709, 711-712, 715, 723, 741, 800-806, 808, 811, 813-821, 823-827, 829-830, 832-833, 835, 844, 846-849, 852, 858-860, 865-866, 868-870, 872-874, 878, 2696-2737 of Table 6.

The complex of claim 65, wherein the base editing system comprises an eA3A_T31AT44A of SEQ ID NO: 3206, said guide RNA comprises a spacer corresponding to any one of the protospacers identified as a sequence of Index Nos. 2725-2726 and 2738-2749 of Table 6.

The complex of claim 65, wherein the base editing system comprises an evoAPOBEC1-BE4max of SEQ ID NO: 3204, said guide RNA comprises a spacer corresponding to any one of the protospacers identified as a sequence of Index Nos. 1-4, 6-7, 9-11, 13, 15-18, 20, 22-27, 32, 35, 40-42, 44, 47-49, 51-53, 55-56, 58, 61-63, 68, 70-72, 74, 76-82, 84-92, 94-98, 100, 104, 108, 111, 116, 121, 125-126, 131, 136, 141-143, 146-148, 150-151, 153, 155-160, 162, 170, 172, 175, 178-180, 183-184, 190, 195, 198, 200-201, 203-204, 206, 210-212, 214, 217, 220-221, 223-227, 229, 231-233, 235-239, 244, 247, 249, 252-258, 263-270, 272-274, 276, 278-279, 281-284, 286-290, 293-294, 296, 298, 300-301, 304, 318, 321, 324-325, 330-333, 338, 340, 342, 346, 349-351, 358, 363, 373, 379-380, 385-389, 411, 423, 425, 427, 431, 433, 438, 441, 445, 448, 454-455, 459, 463, 465, 472, 476, 480, 482-484, 487, 491, 493-494, 503, 510, 514, 517, 521, 535, 540, 542, 544-545, 551-555, 558-564, 567-568, 573-576, 579-582, 588-589, 593, 595-596, 598, 600, 603, 605, 610, 612-617, 620, 622, 625-626, 628, 630-631, 635-641, 644, 651, 653-654, 656, 676, 678-679, 682, 688, 694, 704, 711, 713-715, 717, 720-723, 728-734, 742-743, 745, 747, 750, 752-754, 756-758, 760, 762, 766, 769-773, 775, 777-779, 781-784, 787, 790-794, 798, 800, 803, 805-806, 809, 811-812, 814, 818-819, 824-825, 827, 829, 831, 833, 835, 838-839, 841-842, 847, 850-855, 857-859, 861, 864, 870-873, 875, 878-879 of Table 6.

IX. Editing Methods/Methods of Treatment

The instant disclosure provides methods for the treatment of a subject diagnosed with a disease associated with or caused by a point mutation that may be corrected by a DNA editing base editor provided herein. For example, in some embodiments, a method is provided that comprises administering to a subject having such a disease, e.g., a cancer associated with a point mutation as described above, an effective amount of an adenosine deaminase base editor that corrects the point mutation or introduces a deactivating mutation into a disease-associated gene. In some embodiments, the disease is a proliferative disease. In some embodiments, the disease is a genetic disease. In some embodiments, the disease is a neoplastic disease. In some embodiments, the disease is a metabolic disease. In some embodiments, the disease is a lysosomal storage disease. Other diseases that may be treated by correcting a point mutation or introducing a deactivating mutation into a disease-associated gene will be known to those of skill in the art, and the disclosure is not limited in this respect.

In some embodiments, the deamination of the mutant A results in the codon encoding the wild-type amino acid. In some embodiments, the contacting is in vivo in a subject. In some embodiments, the subject has or has been diagnosed with a disease or disorder. In some embodiments, the disease or disorder is phenylketonuria, von Willebrand disease (vWD), a neoplastic disease associated with a mutant PTEN or BRCA1, or Li-Fraumeni syndrome. A list of exemplary diseases and disorders that may be treated using the base editors described herein is shown in Table 4. Table 4 includes the target gene, the mutation to be corrected, the related disease and the nucleotide sequence of the associated protospacer and PAM.

TABLE 4
List of exemplary diseases that may be treated using the base editors
described herein. The Adenine to be edited in the protospacer is indicated by underlining
and the PAM is indicated in bold.

| Target | | ATCC Cell | | |
|---|---|---|---|---|
| Gene | Mutation | Line | Disease | Protospacer and PAM |
| PTEN | Cys136Tyr | HTB-128 | Cancer | TATATGCATATTTATTACATCGG (SEQ ID NO: 3215) |
| | | | Predisposition | |
| PTEN | Arg233Ter | HTB-13 | Cancer | CCGTCATGTGGGTCCTGAATTGG (SEQ ID NO: 3216) |
| | | | Predisposition | |
| TP53 | Glu258Lys | HTB-65 | Cancer | ACACTGAAAGACTCCAGGTCAGG (SEQ ID NO: 3217) |
| | | | Predisposition | |
| BRCA1 | Gly1738Arg | NA | Cancer | GTCAGAAGAGATGTGGTCAATGG (SEQ ID NO: 88) |
| | | | Predisposition | |
| BRCA1 | 4097-1G > A | NA | Cancer | TTTAAAGTGAAGCAGCATCTGGG (SEQ ID NO: 3218); |
| | | | Predisposition | ATTTAAAGTGAAGCAGCATCTGG (SEQ ID NO: 3219) |
| PAH | Thr380Met | NA | Phenylketonuria | ACTCCATGACAGTGTAATTTTGG (SEQ ID NO: 3220) |
| VWF | Ser1285Phe | NA | von Willebrand | GCCTGGAGAAGCCATCCAGCAGG (SEQ ID NO: 3221) |
| | | | (Hemophilia) | |
| VWF | Arg2535Ter | NA | von Willebrand | CTCAGACACACTCATTGATGAGG (SEQ ID NO: 3222) |
| | | | (Hemophilia) | |
| TP53 | Arg175His | HCC1395 | Li-Fraumeni | GAGGCACTGCCCCCACCATGAGCG (SEQ ID NO: 3223) |
| | | | syndrome | |

Some embodiments provide methods for using the adenine base editors provided herein. In some embodiments, the base editors are used to introduce a point mutation into a nucleic acid by deaminating a target nucleobase, e.g., an A residue. In some embodiments, the deamination of the target nucleobase results in the correction of a genetic defect, e.g., in the correction of a point mutation that leads to a loss of function in a gene product. In some embodiments, the genetic defect is associated with a disease or disorder, e.g., a lysosomal storage disorder or a metabolic disease, such as, for example, type I diabetes. In some embodiments, the methods provided herein are used to introduce a deactivating point mutation into a gene or allele that encodes a gene product that is associated with a disease or disorder. For example, in some embodiments, methods are provided herein that employ a DNA editing base editor to introduce a deactivating point mutation into an oncogene (e.g., in the treatment of a proliferative disease). A deactivating mutation may, in some embodiments, generate a premature stop codon in a coding sequence, which results in the expression of a truncated gene product, e.g., a truncated protein lacking the function of the full-length protein.

In some embodiments, the purpose of the methods provided herein is to restore the function of a dysfunctional gene via genome editing. The nucleobase editing proteins provided herein can be validated for gene editing-based human therapeutics in vitro, e.g., by correcting a disease-associated mutation in human cell culture. It will be understood by the skilled artisan that the nucleobase editing proteins provided herein, e.g., the base editors comprising a nucleic acid programmable DNA binding protein (e.g., Cas9) and an adenosine deaminase domain may be used to correct any single point G to A or C to T mutation. In the first case, deamination of the mutant A to I corrects the mutation, and in the latter case, deamination of the A that is base-paired with the mutant T, followed by a round of replication, corrects the mutation. Exemplary point mutations that may be corrected are listed in Table 4.

The successful correction of point mutations in disease-associated genes and alleles opens up new strategies for gene correction with applications in therapeutics and basic research. Site-specific single-base modification systems like the disclosed fusions of a napDNAbp domain and an adenosine deaminase domain also have applications in “reverse” gene therapy, where certain gene functions are purposely suppressed or abolished. In these cases, site-specifically mutating residues that lead to inactivating mutations in a protein, or mutations that inhibit function of the protein may be used to abolish or inhibit protein function. Without wishing to be bound by any particular theory certain anemias, such as sickle cell anemia, may be treated by inducing expression of hemoglobin, such as fetal hemoglobin, which is typically silenced in adults. As one example, mutating −198T to C in the promoter driving HBG1 and HBG2 gene expression results in increased expression of HBG1 and HBG2. Another example, a class of disorders that results from a G to A mutation in a gene is iron storage disorders, where the HFE gene comprises a G to A mutation that results in expression of a C282Y mutant HFE protein. A list of additional exemplary diseases and disorders that may be treated using the base editors described herein is shown in Table 4, above.

The present disclosure provides methods for the treatment of additional diseases or disorders, e.g., diseases or disorders that are associated or caused by a point mutation that may be corrected by deaminase-mediated gene editing. Some such diseases are described herein, and additional suitable diseases that may be treated with the strategies and base editors provided herein will be apparent to those of skill in the art based on the instant disclosure. Exemplary suitable diseases and disorders are listed below. Exemplary suitable diseases and disorders include, without limitation: 2-methyl-3-hydroxybutyric aciduria; 3 beta-Hydroxysteroid dehydrogenase deficiency; 3-Methylglutaconic aciduria; 3-Oxo-5 alpha-steroid delta 4-dehydrogenase deficiency; 46,XY sex reversal, type 1, 3, and 5; 5-Oxoprolinase deficiency; 6-pyruvoyl-tetrahydropterin synthase deficiency; Aarskog syndrome; Aase syndrome; Achondrogenesis type 2; Achromatopsia 2 and 7; Acquired long QT syndrome; Acrocallosal syndrome, Schinzel type; Acrocapitofemoral dysplasia; Acrodysostosis 2, with or without hormone resistance; Acroerythrokeratoderma; Acromicric dysplasia; Acth-independent macronodular adrenal hyperplasia 2; Activated PI3K-delta syndrome; Acute intermittent porphyria; deficiency of Acyl-CoA dehydrogenase family, member 9; Adams-Oliver syndrome 5 and 6; Adenine phosphoribosyltransferase deficiency; Adenylate kinase deficiency; hemolytic anemia due to Adenylosuccinate lyase deficiency; Adolescent nephronophthisis; Renal-hepatic-pancreatic dysplasia; Meckel syndrome type 7; Adrenoleukodystrophy; Adult junctional epidermolysis bullosa; Epidermolysis bullosa, junctional, localisata variant; Adult neuronal ceroid lipofuscinosis; Adult neuronal ceroid lipofuscinosis; Adult onset ataxia with oculomotor apraxia; ADULT syndrome; Afibrinogenemia and congenital Afibrinogenemia; autosomal recessive Agammaglobulinemia 2; Age-related macular degeneration 3, 6, 11, and 12; Aicardi Goutieres syndromes 1, 4, and 5; Chilbain lupus 1; Alagille syndromes 1 and 2; Alexander disease; Alkaptonuria; Allan-Herndon-Dudley syndrome; Alopecia universalis congenital; Alpers encephalopathy; Alpha-1-antitrypsin deficiency; autosomal dominant, autosomal recessive, and X-linked recessive Alport syndromes; Alzheimer disease, familial, 3, with spastic paraparesis and apraxia; Alzheimer disease, types, 1, 3, and 4; hypocalcification type and hypomaturation type, IIA1 Amelogenesis imperfecta; Aminoacylase 1 deficiency; Amish infantile epilepsy syndrome; Amyloidogenic transthyretin amyloidosis; Amyloid Cardiomyopathy, Transthyretin-related; Cardiomyopathy; Amyotrophic lateral sclerosis types 1, 6, 15 (with or without frontotemporal dementia), 22 (with or without frontotemporal dementia), and 10; Frontotemporal dementia with TDP43 inclusions, TARDBP-related; Andermann syndrome; Andersen Tawil syndrome; Congenital long QT syndrome; Anemia, nonspherocytic hemolytic, due to G6PD deficiency; Angelman syndrome; Severe neonatal-onset encephalopathy with microcephaly; susceptibility to Autism, X-linked 3; Angiopathy, hereditary, with nephropathy, aneurysms, and muscle cramps; Angiotensin i-converting enzyme, benign serum increase; Aniridia, cerebellar ataxia, and mental retardation; Anonychia; Antithrombin III deficiency; Antley-Bixler syndrome with genital anomalies and disordered steroidogenesis; Aortic aneurysm, familial thoracic 4, 6, and 9; Thoracic aortic aneurysms and aortic dissections; Multisystemic smooth muscle dysfunction syndrome; Moyamoya disease 5; Aplastic anemia; Apparent mineralocorticoid excess; Arginase deficiency; Argininosuccinate lyase deficiency; Aromatase deficiency; Arrhythmogenic right ventricular cardiomyopathy types 5, 8, and 10; Primary familial hypertrophic cardiomyopathy; Arthrogryposis multiplex congenita, distal, X-linked; Arthrogryposis renal dysfunction cholestasis syndrome; Arthrogryposis, renal dysfunction, and cholestasis 2; Asparagine synthetase deficiency; Abnormality of neuronal migration; Ataxia with vitamin E deficiency; Ataxia, sensory, autosomal dominant; Ataxia-telangiectasia syndrome; Hereditary cancer-predisposing syndrome; Atransferrinemia; Atrial fibrillation, familial, 11, 12, 13, and 16; Atrial septal defects 2, 4, and 7 (with or without atrioventricular conduction defects); Atrial standstill 2; Atrioventricular septal defect 4; Atrophia bulborum hereditaria; ATR-X syndrome; Auriculocondylar syndrome 2; Autoimmune disease, multisystem, infantile-onset; Autoimmune lymphoproliferative syndrome, type 1a; Autosomal dominant hypohidrotic ectodermal dysplasia; Autosomal dominant progressive external ophthalmoplegia with mitochondrial DNA deletions 1 and 3; Autosomal dominant torsion dystonia 4; Autosomal recessive centronuclear myopathy; Autosomal recessive congenital ichthyosis 1, 2, 3, 4A, and 4B; Autosomal recessive cutis laxa type IA and 1B; Autosomal recessive hypohidrotic ectodermal dysplasia syndrome; Ectodermal dysplasia 11b; hypohidrotic/hair/tooth type, autosomal recessive; Autosomal recessive hypophosphatemic bone disease; Axenfeld-Rieger syndrome type 3; Bainbridge-Ropers syndrome; Bannayan-Riley-Ruvalcaba syndrome; PTEN hamartoma tumor syndrome; Baraitser-Winter syndromes 1 and 2; Barakat syndrome; Bardet-Biedl syndromes 1, 11, 16, and 19; Bare lymphocyte syndrome type 2, complementation group E; Bartter syndrome antenatal type 2; Bartter syndrome types 3, 3 with hypocalciuria, and 4; Basal ganglia calcification, idiopathic, 4; Beaded hair; Benign familial hematuria; Benign familial neonatal seizures 1 and 2; Seizures, benign familial neonatal, 1, and/or myokymia; Seizures, Early infantile epileptic encephalopathy 7; Benign familial neonatal-infantile seizures; Benign hereditary chorea; Benign scapuloperoneal muscular dystrophy with cardiomyopathy; Bernard-Soulier syndrome, types A1 and A2 (autosomal dominant); Bestrophinopathy, autosomal recessive; beta Thalassemia; Bethlem myopathy and Bethlem myopathy 2; Bietti crystalline corneoretinal dystrophy; Bile acid synthesis defect, congenital, 2; Biotinidase deficiency; Birk Barel mental retardation dysmorphism syndrome; Blepharophimosis, ptosis, and epicanthus inversus; Bloom syndrome; Borjeson-Forssman-Lehmann syndrome; Boucher Neuhauser syndrome; Brachydactyly types A1 and A2; Brachydactyly with hypertension; Brain small vessel disease with hemorrhage; Branched-chain ketoacid dehydrogenase kinase deficiency; Branchiootic syndromes 2 and 3; Breast cancer, early-onset; Breast-ovarian cancer, familial 1, 2, and 4; Brittle cornea syndrome 2; Brody myopathy; Bronchiectasis with or without elevated sweat chloride 3; Brown-Vialetto-Van laere syndrome and Brown-Vialetto-Van Laere syndrome 2; Brugada syndrome; Brugada syndrome 1; Ventricular fibrillation; Paroxysmal familial ventricular fibrillation; Brugada syndrome and Brugada syndrome 4; Long QT syndrome; Sudden cardiac death; Bull eye macular dystrophy; Stargardt disease 4; Cone-rod dystrophy 12; Bullous ichthyosiform erythroderma; Burn-Mckeown syndrome; Candidiasis, familial, 2, 5, 6, and 8; Carbohydrate-deficient glycoprotein syndrome type I and II; Carbonic anhydrase VA deficiency, hyperammonemia due to; Carcinoma of colon; Cardiac arrhythmia; Long QT syndrome, LQT1 subtype; Cardioencephalomyopathy, fatal infantile, due to cytochrome c oxidase deficiency; Cardiofaciocutaneous syndrome; Cardiomyopathy; Danon disease; Hypertrophic cardiomyopathy; Left ventricular noncompaction cardiomyopathy; Carnevale syndrome; Carney complex, type 1; Carnitine acylcarnitine translocase deficiency; Carnitine palmitoyltransferase I, II, II (late onset), and II (infantile) deficiency; Cataract 1, 4, autosomal dominant, autosomal dominant, multiple types, with microcornea, coppock-like, juvenile, with microcornea and glucosuria, and nuclear diffuse nonprogressive; Catecholaminergic polymorphic ventricular tachycardia; Caudal regression syndrome; Cd8 deficiency, familial; Central core disease; Centromeric instability of chromosomes 1, 9 and 16 and immunodeficiency; Cerebellar ataxia infantile with progressive external ophthalmoplegi and Cerebellar ataxia, mental retardation, and dysequilibrium syndrome 2; Cerebral amyloid angiopathy, APP-related; Cerebral autosomal dominant and recessive arteriopathy with subcortical infarcts and leukoencephalopathy; Cerebral cavernous malformations 2; Cerebrooculofacioskeletal syndrome 2; Cerebro-oculo-facio-skeletal syndrome; Cerebroretinal microangiopathy with calcifications and cysts; Ceroid lipofuscinosis neuronal 2, 6, 7, and 10; Ch\xc3\xa9diak-Higashi syndrome, Chediak-Higashi syndrome, adult type; Charcot-Marie-Tooth disease types 1B, 2B2, 2C, 2F, 2I, 2U (axonal), 1C (demyelinating), dominant intermediate C, recessive intermediate A, 2A2, 4C, 4D, 4H, IF, IVF, and X; Scapuloperoneal spinal muscular atrophy; Distal spinal muscular atrophy, congenital nonprogressive; Spinal muscular atrophy, distal, autosomal recessive, 5; CHARGE association; Childhood hypophosphatasia; Adult hypophosphatasia; Cholecystitis; Progressive familial intrahepatic cholestasis 3; Cholestasis, intrahepatic, of pregnancy 3; Cholestanol storage disease; Cholesterol monooxygenase (side-chain cleaving) deficiency; Chondrodysplasia Blomstrand type; Chondrodysplasia punctata 1, X-linked recessive and 2 X-linked dominant; CHOPS syndrome; Chronic granulomatous disease, autosomal recessive cytochrome b-positive, types 1 and 2; Chudley-McCullough syndrome; Ciliary dyskinesia, primary, 7, 11, 15, 20 and 22; Citrullinemia type I; Citrullinemia type I and II; Cleidocranial dysostosis; C-like syndrome; Cockayne syndrome type A, Coenzyme Q10 deficiency, primary 1, 4, and 7; Coffin Siris/Intellectual Disability; Coffin-Lowry syndrome; Cohen syndrome, Cold-induced sweating syndrome 1; COLE-CARPENTER SYNDROME 2; Combined cellular and humoral immune defects with granulomas; Combined d-2- and 1-2-hydroxyglutaric aciduria; Combined malonic and methylmalonic aciduria; Combined oxidative phosphorylation deficiencies 1, 3, 4, 12, 15, and 25; Combined partial and complete 17-alpha-hydroxylase/17,20-lyase deficiency; Common variable immunodeficiency 9; Complement component 4, partial deficiency of, due to dysfunctional c1 inhibitor; Complement factor B deficiency; Cone monochromatism; Cone-rod dystrophy 2 and 6; Cone-rod dystrophy amelogenesis imperfecta; Congenital adrenal hyperplasia and Congenital adrenal hypoplasia, X-linked; Congenital amegakaryocytic thrombocytopenia; Congenital aniridia; Congenital central hypoventilation; Hirschsprung disease 3; Congenital contractural arachnodactyly; Congenital contractures of the limbs and face, hypotonia, and developmental delay; Congenital disorder of glycosylation types 1B, 1D, 1G, 1H, 1J, 1K, 1N, 1P, 2C, 2J, 2K, IIm; Congenital dyserythropoietic anemia, type I and II; Congenital ectodermal dysplasia of face; Congenital erythropoietic porphyria; Congenital generalized lipodystrophy type 2; Congenital heart disease, multiple types, 2; Congenital heart disease; Interrupted aortic arch; Congenital lipomatous overgrowth, vascular malformations, and epidermal nevi; Non-small cell lung cancer; Neoplasm of ovary; Cardiac conduction defect, nonspecific; Congenital microvillous atrophy; Congenital muscular dystrophy; Congenital muscular dystrophy due to partial LAMA2 deficiency; Congenital muscular dystrophy-dystroglycanopathy with brain and eye anomalies, types A2, A7, A8, A11, and A14; Congenital muscular dystrophy-dystroglycanopathy with mental retardation, types B2, B3, B5, and B15; Congenital muscular dystrophy-dystroglycanopathy without mental retardation, type B5; Congenital muscular hypertrophy-cerebral syndrome; Congenital myasthenic syndrome, acetazolamide-responsive; Congenital myopathy with fiber type disproportion; Congenital ocular coloboma; Congenital stationary night blindness, type 1A, 1B, 1C, 1E, 1F, and 2A; Coproporphyria; Cornea plana 2; Corneal dystrophy, Fuchs endothelial, 4; Corneal endothelial dystrophy type 2; Corneal fragility keratoglobus, blue sclerae and joint hypermobility; Cornelia de Lange syndromes 1 and 5; Coronary artery disease, autosomal dominant 2; Coronary heart disease; Hyperalphalipoproteinemia 2; Cortical dysplasia, complex, with other brain malformations 5 and 6; Cortical malformations, occipital; Corticosteroid-binding globulin deficiency; Corticosterone methyloxidase type 2 deficiency; Costello syndrome; Cowden syndrome 1; Coxa plana; Craniodiaphyseal dysplasia, autosomal dominant; Craniosynostosis 1 and 4; Craniosynostosis and dental anomalies; Creatine deficiency, X-linked; Crouzon syndrome; Cryptophthalmos syndrome; Cryptorchidism, unilateral or bilateral; Cushing symphalangism; Cutaneous malignant melanoma 1; Cutis laxa with osteodystrophy and with severe pulmonary, gastrointestinal, and urinary abnormalities; Cyanosis, transient neonatal and atypical nephropathic; Cystic fibrosis; Cystinuria; Cytochrome c oxidase i deficiency; Cytochrome-c oxidase deficiency; D-2-hydroxyglutaric aciduria 2; Darier disease, segmental; Deafness with labyrinthine aplasia microtia and microdontia (LAMM); Deafness, autosomal dominant 3a, 4, 12, 13, 15, autosomal dominant nonsyndromic sensorineural 17, 20, and 65; Deafness, autosomal recessive 1A, 2, 3, 6, 8, 9, 12, 15, 16, 18b, 22, 28, 31, 44, 49, 63, 77, 86, and 89; Deafness, cochlear, with myopia and intellectual impairment, without vestibular involvement, autosomal dominant, X-linked 2; Deficiency of 2-methylbutyryl-CoA dehydrogenase; Deficiency of 3-hydroxyacyl-CoA dehydrogenase; Deficiency of alpha-mannosidase; Deficiency of aromatic-L-amino-acid decarboxylase; Deficiency of bisphosphoglycerate mutase; Deficiency of butyryl-CoA dehydrogenase; Deficiency of ferroxidase; Deficiency of galactokinase; Deficiency of guanidinoacetate methyltransferase; Deficiency of hyaluronoglucosaminidase; Deficiency of ribose-5-phosphate isomerase; Deficiency of steroid 11-beta-monooxygenase; Deficiency of UDPglucose-hexose-1-phosphate uridylyltransferase; Deficiency of xanthine oxidase; Dejerine-Sottas disease; Charcot-Marie-Tooth disease, types ID and IVF; Dejerine-Sottas syndrome, autosomal dominant; Dendritic cell, monocyte, B lymphocyte, and natural killer lymphocyte deficiency; Desbuquois dysplasia 2; Desbuquois syndrome; DFNA 2 Nonsyndromic Hearing Loss; Diabetes mellitus and insipidus with optic atrophy and deafness; Diabetes mellitus, type 2, and insulin-dependent, 20; Diamond-Blackfan anemia 1, 5, 8, and 10; Diarrhea 3 (secretory sodium, congenital, syndromic) and 5 (with tufting enteropathy, congenital); Dicarboxylic aminoaciduria; Diffuse palmoplantar keratoderma, Bothnian type; Digitorenocerebral syndrome; Dihydropteridine reductase deficiency; Dilated cardiomyopathy 1A, 1AA, 1C, 1G, 1BB, 1DD, 1FF, 1HH, 1I, 1KK, 1N, 1S, 1Y, and 3B; Left ventricular noncompaction 3; Disordered steroidogenesis due to cytochrome p450 oxidoreductase deficiency; Distal arthrogryposis type 2B; Distal hereditary motor neuronopathy type 2B; Distal myopathy Markesbery-Griggs type; Distal spinal muscular atrophy, X-linked 3; Distichiasis-lymphedema syndrome; Dominant dystrophic epidermolysis bullosa with absence of skin; Dominant hereditary optic atrophy; Donnai Barrow syndrome; Dopamine beta hydroxylase deficiency; Dopamine receptor d2, reduced brain density of, Dowling-degos disease 4; Doyne honeycomb retinal dystrophy; Malattia leventinese; Duane syndrome type 2; Dubin-Johnson syndrome; Duchenne muscular dystrophy; Becker muscular dystrophy; Dysfibrinogenemia; Dyskeratosis congenita autosomal dominant and autosomal dominant, 3; Dyskeratosis congenita, autosomal recessive, 1, 3, 4, and 5; Dyskeratosis congenita X-linked; Dyskinesia, familial, with facial myokymia; Dysplasminogenemia; Dystonia 2 (torsion, autosomal recessive), 3 (torsion, X-linked), 5 (Dopa-responsive type), 10, 12, 16, 25, 26 (Myoclonic); Seizures, benign familial infantile, 2; Early infantile epileptic encephalopathy 2, 4, 7, 9, 10, 11, 13, and 14; Atypical Rett syndrome; Early T cell progenitor acute lymphoblastic leukemia; Ectodermal dysplasia skin fragility syndrome; Ectodermal dysplasia-syndactyly syndrome 1; Ectopia lentis, isolated autosomal recessive and dominant; Ectrodactyly, ectodermal dysplasia, and cleft lip/palate syndrome 3; Ehlers-Danlos syndrome type 7 (autosomal recessive), classic type, type 2 (progeroid), hydroxylysine-deficient, type 4, type 4 variant, and due to tenascin-X deficiency; Eichsfeld type congenital muscular dystrophy; Endocrine-cerebroosteodysplasia; Enhanced s-cone syndrome; Enlarged vestibular aqueduct syndrome; Enterokinase deficiency; Epidermodysplasia verruciformis; Epidermolysa bullosa simplex and limb girdle muscular dystrophy, simplex with mottled pigmentation, simplex with pyloric atresia, simplex, autosomal recessive, and with pyloric atresia; Epidermolytic palmoplantar keratoderma; Familial febrile seizures 8; Epilepsy, childhood absence 2, 12 (idiopathic generalized, susceptibility to) 5 (nocturnal frontal lobe), nocturnal frontal lobe type 1, partial, with variable foci, progressive myoclonic 3, and X-linked, with variable learning disabilities and behavior disorders; Epileptic encephalopathy, childhood-onset, early infantile, 1, 19, 23, 25, 30, and 32; Epiphyseal dysplasia, multiple, with myopia and conductive deafness; Episodic ataxia type 2; Episodic pain syndrome, familial, 3; Epstein syndrome; Fechtner syndrome; Erythropoietic protoporphyria; Estrogen resistance; Exudative vitreoretinopathy 6; Fabry disease and Fabry disease, cardiac variant; Factor H, VII, X, v and factor viii, combined deficiency of 2, xiii, a subunit, deficiency; Familial adenomatous polyposis 1 and 3; Familial amyloid nephropathy with urticaria and deafness; Familial cold urticarial; Familial aplasia of the vermis; Familial benign pemphigus; Familial cancer of breast; Breast cancer, susceptibility to; Osteosarcoma; Pancreatic cancer 3; Familial cardiomyopathy; Familial cold autoinflammatory syndrome 2; Familial colorectal cancer; Familial exudative vitreoretinopathy, X-linked; Familial hemiplegic migraine types 1 and 2; Familial hypercholesterolemia; Familial hypertrophic cardiomyopathy 1, 2, 3, 4, 7, 10, 23 and 24; Familial hypokalemia-hypomagnesemia; Familial hypoplastic, glomerulocystic kidney; Familial infantile myasthenia; Familial juvenile gout; Familial Mediterranean fever and Familial mediterranean fever, autosomal dominant; Familial porencephaly; Familial porphyria cutanea tarda; Familial pulmonary capillary hemangiomatosis; Familial renal glucosuria; Familial renal hypouricemia; Familial restrictive cardiomyopathy 1; Familial type 1 and 3 hyperlipoproteinemia; Fanconi anemia, complementation group E, I, N, and 0; Fanconi-Bickel syndrome; Favism, susceptibility to; Febrile seizures, familial, 11; Feingold syndrome 1; Fetal hemoglobin quantitative trait locus 1; FG syndrome and FG syndrome 4; Fibrosis of extraocular muscles, congenital, 1, 2, 3a (with or without extraocular involvement), 3b; Fish-eye disease; Fleck corneal dystrophy; Floating-Harbor syndrome; Focal epilepsy with speech disorder with or without mental retardation; Focal segmental glomerulosclerosis 5; Forebrain defects; Frank Ter Haar syndrome; Borrone Di Rocco Crovato syndrome; Frasier syndrome; Wilms tumor 1; Freeman-Sheldon syndrome; Frontometaphyseal dysplasia land 3; Frontotemporal dementia; Frontotemporal dementia and/or amyotrophic lateral sclerosis 3 and 4; Frontotemporal Dementia Chromosome 3-Linked and Frontotemporal dementia ubiquitin-positive; Fructose-biphosphatase deficiency; Fuhrmann syndrome; Gamma-aminobutyric acid transaminase deficiency; Gamstorp-Wohlfart syndrome; Gaucher disease type 1 and Subacute neuronopathic; Gaze palsy, familial horizontal, with progressive scoliosis; Generalized dominant dystrophic epidermolysis bullosa; Generalized epilepsy with febrile seizures plus 3, type 1, type 2; Epileptic encephalopathy Lennox-Gastaut type; Giant axonal neuropathy; Glanzmann thrombasthenia; Glaucoma 1, open angle, e, F, and G; Glaucoma 3, primary congenital, d; Glaucoma, congenital and Glaucoma, congenital, Coloboma; Glaucoma, primary open angle, juvenile-onset; Glioma susceptibility 1; Glucose transporter type 1 deficiency syndrome; Glucose-6-phosphate transport defect; GLUT1 deficiency syndrome 2; Epilepsy, idiopathic generalized, susceptibility to, 12; Glutamate formiminotransferase deficiency; Glutaric acidemia IIA and IIB; Glutaric aciduria, type 1; Gluthathione synthetase deficiency; Glycogen storage disease 0 (muscle), II (adult form), IXa2, IXc, type 1A; type II, type IV, IV (combined hepatic and myopathic), type V, and type VI; Goldmann-Favre syndrome; Gordon syndrome; Gorlin syndrome; Holoprosencephaly sequence; Holoprosencephaly 7; Granulomatous disease, chronic, X-linked, variant; Granulosa cell tumor of the ovary; Gray platelet syndrome; Griscelli syndrome type 3; Groenouw corneal dystrophy type I; Growth and mental retardation, mandibulofacial dysostosis, microcephaly, and cleft palate; Growth hormone deficiency with pituitary anomalies; Growth hormone insensitivity with immunodeficiency; GTP cyclohydrolase I deficiency; Hajdu-Cheney syndrome; Hand foot uterus syndrome; Hearing impairment; Hemangioma, capillary infantile; Hematologic neoplasm; Hemochromatosis type 1, 2B, and 3; Microvascular complications of diabetes 7; Transferrin serum level quantitative trait locus 2; Hemoglobin H disease, nondeletional; Hemolytic anemia, nonspherocytic, due to glucose phosphate isomerase deficiency; Hemophagocytic lymphohistiocytosis, familial, 2; Hemophagocytic lymphohistiocytosis, familial, 3; Heparin cofactor II deficiency; Hereditary acrodermatitis enteropathica; Hereditary breast and ovarian cancer syndrome; Ataxia-telangiectasia-like disorder; Hereditary diffuse gastric cancer; Hereditary diffuse leukoencephalopathy with spheroids; Hereditary factors II, IX, VIII deficiency disease; Hereditary hemorrhagic telangiectasia type 2; Hereditary insensitivity to pain with anhidrosis; Hereditary lymphedema type I; Hereditary motor and sensory neuropathy with optic atrophy; Hereditary myopathy with early respiratory failure; Hereditary neuralgic amyotrophy; Hereditary Nonpolyposis Colorectal Neoplasms; Lynch syndrome I and II; Hereditary pancreatitis; Pancreatitis, chronic, susceptibility to; Hereditary sensory and autonomic neuropathy type IIB amd IIA; Hereditary sideroblastic anemia; Hermansky-Pudlak syndrome 1, 3, 4, and 6; Heterotaxy, visceral, 2, 4, and 6, autosomal; Heterotaxy, visceral, X-linked; Heterotopia; Histiocytic medullary reticulosis; Histiocytosis-lymphadenopathy plus syndrome; Holocarboxylase synthetase deficiency; Holoprosencephaly 2, 3, 7, and 9; Holt-Oram syndrome; Homocysteinemia due to MTHFR deficiency, CBS deficiency, and Homocystinuria, pyridoxine-responsive; Homocystinuria-Megaloblastic anemia due to defect in cobalamin metabolism, cblE complementation type; Howel-Evans syndrome; Hurler syndrome; Hutchinson-Gilford syndrome; Hydrocephalus; Hyperammonemia, type III; Hypercholesterolaemia and Hypercholesterolemia, autosomal recessive; Hyperekplexia 2 and Hyperekplexia hereditary; Hyperferritinemia cataract syndrome; Hyperglycinuria; Hyperimmunoglobulin D with periodic fever; Mevalonic aciduria; Hyperimmunoglobulin E syndrome; Hyperinsulinemic hypoglycemia familial 3, 4, and 5; Hyperinsulinism-hyperammonemia syndrome; Hyperlysinemia; Hypermanganesemia with dystonia, polycythemia and cirrhosis; Hyperornithinemia-hyperammonemia-homocitrullinuria syndrome; Hyperparathyroidism 1 and 2; Hyperparathyroidism, neonatal severe; Hyperphenylalaninemia, bh4-deficient, a, due to partial pts deficiency, BH4-deficient, D, and non-pku; Hyperphosphatasia with mental retardation syndrome 2, 3, and 4; Hypertrichotic osteochondrodysplasia; Hypobetalipoproteinemia, familial, associated with apob32; Hypocalcemia, autosomal dominant 1; Hypocalciuric hypercalcemia, familial, types 1 and 3; Hypochondrogenesis; Hypochromic microcytic anemia with iron overload; Hypoglycemia with deficiency of glycogen synthetase in the liver; Hypogonadotropic hypogonadism 11 with or without anosmia; Hypohidrotic ectodermal dysplasia with immune deficiency; Hypohidrotic X-linked ectodermal dysplasia; Hypokalemic periodic paralysis 1 and 2; Hypomagnesemia 1, intestinal; Hypomagnesemia, seizures, and mental retardation; Hypomyelinating leukodystrophy 7; Hypoplastic left heart syndrome; Atrioventricular septal defect and common atrioventricular junction; Hypospadias 1 and 2, X-linked; Hypothyroidism, congenital, nongoitrous, 1; Hypotrichosis 8 and 12; Hypotrichosis-lymphedema-telangiectasia syndrome; I blood group system; Ichthyosis bullosa of Siemens; Ichthyosis exfoliativa; Ichthyosis prematurity syndrome; Idiopathic basal ganglia calcification 5; Idiopathic fibrosing alveolitis, chronic form; Dyskeratosis congenita, autosomal dominant, 2 and 5; Idiopathic hypercalcemia of infancy; Immune dysfunction with T-cell inactivation due to calcium entry defect 2; Immunodeficiency 15, 16, 19, 30, 31C, 38, 40, 8, due to defect in cd3-zeta, with hyper IgM type 1 and 2, and X-Linked, with magnesium defect, Epstein-Barr virus infection, and neoplasia; Immunodeficiency-centromeric instability-facial anomalies syndrome 2; Inclusion body myopathy 2 and 3; Nonaka myopathy; Infantile convulsions and paroxysmal choreoathetosis, familial; Infantile cortical hyperostosis; Infantile GM1 gangliosidosis; Infantile hypophosphatasia; Infantile nephronophthisis; Infantile nystagmus, X-linked; Infantile Parkinsonism-dystonia; Infertility associated with multi-tailed spermatozoa and excessive DNA; Insulin resistance; Insulin-resistant diabetes mellitus and acanthosis nigricans; Insulin-dependent diabetes mellitus secretory diarrhea syndrome; Interstitial nephritis, karyomegalic; Intrauterine growth retardation, metaphyseal dysplasia, adrenal hypoplasia congenita, and genital anomalies; Iodotyrosyl coupling defect; IRAK4 deficiency; Iridogoniodysgenesis dominant type and type 1; Iron accumulation in brain; Ischiopatellar dysplasia; Islet cell hyperplasia; Isolated 17,20-lyase deficiency; Isolated lutropin deficiency; Isovaleryl-CoA dehydrogenase deficiency; Jankovic Rivera syndrome; Jervell and Lange-Nielsen syndrome 2; Joubert syndrome 1, 6, 7, 9/15 (digenic), 14, 16, and 17, and Orofaciodigital syndrome xiv; Junctional epidermolysis bullosa gravis of Herlitz; Juvenile GM>1<gangliosidosis; Juvenile polyposis syndrome; Juvenile polyposis/hereditary hemorrhagic telangiectasia syndrome; Juvenile retinoschisis; Kabuki make-up syndrome; Kallmann syndrome 1, 2, and 6; Delayed puberty; Kanzaki disease; Karak syndrome; Kartagener syndrome; Kenny-Caffey syndrome type 2; Keppen-Lubinsky syndrome; Keratoconus 1; Keratosis follicularis; Keratosis palmoplantaris striata 1; Kindler syndrome; L-2-hydroxyglutaric aciduria; Larsen syndrome, dominant type; Lattice corneal dystrophy Type III; Leber amaurosis; Zellweger syndrome; Peroxisome biogenesis disorders; Zellweger syndrome spectrum; Leber congenital amaurosis 11, 12, 13, 16, 4, 7, and 9; Leber optic atrophy; Aminoglycoside-induced deafness; Deafness, nonsyndromic sensorineural, mitochondrial; Left ventricular noncompaction 5; Left-right axis malformations; Leigh disease; Mitochondrial short-chain Enoyl-CoA Hydratase 1 deficiency; Leigh syndrome due to mitochondrial complex I deficiency; Leiner disease; Leri Weill dyschondrosteosis; Lethal congenital contracture syndrome 6; Leukocyte adhesion deficiency type I and III; Leukodystrophy, Hypomyelinating, 11 and 6; Leukoencephalopathy with ataxia, with Brainstem and Spinal Cord Involvement and Lactate Elevation, with vanishing white matter, and progressive, with ovarian failure; Leukonychia totalis; Lewy body dementia; Lichtenstein-Knorr Syndrome; Li-Fraumeni syndrome 1; Lig4 syndrome; Limb-girdle muscular dystrophy, type 1B, 2A, 2B, 2D, C1, C5, C9, C14; Congenital muscular dystrophy-dystroglycanopathy with brain and eye anomalies, type A14 and B14; Lipase deficiency combined; Lipid proteinosis; Lipodystrophy, familial partial, type 2 and 3; Lissencephaly 1, 2 (X-linked), 3, 6 (with microcephaly), X-linked; Subcortical laminar heterotopia, X-linked; Liver failure acute infantile; Loeys-Dietz syndrome 1, 2, 3; Long QT syndrome 1, 2, 2/9, 2/5, (digenic), 3, 5 and 5, acquired, susceptibility to; Lung cancer; Lymphedema, hereditary, id; Lymphedema, primary, with myelodysplasia; Lymphoproliferative syndrome 1, 1 (X-linked), and 2; Lysosomal acid lipase deficiency; Macrocephaly, macrosomia, facial dysmorphism syndrome; Macular dystrophy, vitelliform, adult-onset; Malignant hyperthermia susceptibility type 1; Malignant lymphoma, non-Hodgkin; Malignant melanoma; Malignant tumor of prostate; Mandibuloacral dysostosis; Mandibuloacral dysplasia with type A or B lipodystrophy, atypical; Mandibulofacial dysostosis, Treacher Collins type, autosomal recessive; Mannose-binding protein deficiency; Maple syrup urine disease type 1A and type 3; Marden Walker like syndrome; Marfan syndrome; Marinesco-Sj\xc3\xb6gren syndrome; Martsolf syndrome; Maturity-onset diabetes of the young, type 1, type 2, type 11, type 3, and type 9; May-Hegglin anomaly; MYH9 related disorders; Sebastian syndrome; McCune-Albright syndrome; Somatotroph adenoma; Sex cord-stromal tumor; Cushing syndrome; McKusick Kaufman syndrome; McLeod neuroacanthocytosis syndrome; Meckel-Gruber syndrome; Medium-chain acyl-coenzyme A dehydrogenase deficiency; Medulloblastoma; Megalencephalic leukoencephalopathy with subcortical cysts land 2a; Megalencephaly cutis marmorata telangiectatica congenital; PIK3CA Related Overgrowth Spectrum; Megalencephaly-polymicrogyria-polydactyly-hydrocephalus syndrome 2; Megaloblastic anemia, thiamine-responsive, with diabetes mellitus and sensorineural deafness; Meier-Gorlin syndromes land 4; Melnick-Needles syndrome; Meningioma; Mental retardation, X-linked, 3, 21, 30, and 72; Mental retardation and microcephaly with pontine and cerebellar hypoplasia; Mental retardation X-linked syndromic 5; Mental retardation, anterior maxillary protrusion, and strabismus; Mental retardation, autosomal dominant 12, 13, 15, 24, 3, 30, 4, 5, 6, and 9; Mental retardation, autosomal recessive 15, 44, 46, and 5; Mental retardation, stereotypic movements, epilepsy, and/or cerebral malformations; Mental retardation, syndromic, Claes-Jensen type, X-linked; Mental retardation, X-linked, nonspecific, syndromic, Hedera type, and syndromic, wu type; Merosin deficient congenital muscular dystrophy; Metachromatic leukodystrophy juvenile, late infantile, and adult types; Metachromatic leukodystrophy; Metatrophic dysplasia; Methemoglobinemia types I and 2; Methionine adenosyltransferase deficiency, autosomal dominant; Methylmalonic acidemia with homocystinuria, Methylmalonic aciduria cblB type, Methylmalonic aciduria due to methylmalonyl-CoA mutase deficiency; METHYLMALONIC ACIDURIA, mut(0) TYPE; Microcephalic osteodysplastic primordial dwarfism type 2; Microcephaly with or without chorioretinopathy, lymphedema, or mental retardation; Microcephaly, hiatal hernia and nephrotic syndrome; Microcephaly; Hypoplasia of the corpus callosum; Spastic paraplegia 50, autosomal recessive; Global developmental delay; CNS hypomyelination; Brain atrophy; Microcephaly, normal intelligence and immunodeficiency; Microcephaly-capillary malformation syndrome; Microcytic anemia; Microphthalmia syndromic 5, 7, and 9; Microphthalmia, isolated 3, 5, 6, 8, and with coloboma 6; Microspherophakia; Migraine, familial basilar; Miller syndrome; Minicore myopathy with external ophthalmoplegia; Myopathy, congenital with cores; Mitchell-Riley syndrome; mitochondrial 3-hydroxy-3-methylglutaryl-CoA synthase deficiency; Mitochondrial complex I, II, III, III (nuclear type 2, 4, or 8) deficiency; Mitochondrial DNA depletion syndrome 11, 12 (cardiomyopathic type), 2, 4B (MNGIE type), 8B (MNGIE type); Mitochondrial DNA-depletion syndrome 3 and 7, hepatocerebral types, and 13 (encephalomyopathic type); Mitochondrial phosphate carrier and pyruvate carrier deficiency; Mitochondrial trifunctional protein deficiency; Long-chain 3-hydroxyacyl-CoA dehydrogenase deficiency; Miyoshi muscular dystrophy 1; Myopathy, distal, with anterior tibial onset; Mohr-Tranebjaerg syndrome; Molybdenum cofactor deficiency, complementation group A; Mowat-Wilson syndrome; Mucolipidosis III Gamma; Mucopolysaccharidosis type VI, type VI (severe), and type VII; Mucopolysaccharidosis, MPS-I-H/S, MPS-II, MPS-III-A, MPS-III-B, MPS-III-C, MPS-IV-A, MPS-IV-B; Retinitis Pigmentosa 73; Gangliosidosis GM1 type1 (with cardiac involvement) 3; Multicentric osteolysis nephropathy; Multicentric osteolysis, nodulosis and arthropathy; Multiple congenital anomalies; Atrial septal defect 2; Multiple congenital anomalies-hypotonia-seizures syndrome 3; Multiple Cutaneous and Mucosal Venous Malformations; Multiple endocrine neoplasia, types land 4; Multiple epiphyseal dysplasia 5 or Dominant; Multiple gastrointestinal atresias; Multiple pterygium syndrome Escobar type; Multiple sulfatase deficiency; Multiple synostoses syndrome 3; Muscle AMP deaminase deficiency; Muscle eye brain disease; Muscular dystrophy, congenital, megaconial type; Myasthenia, familial infantile, 1; Myasthenic Syndrome, Congenital, 11, associated with acetylcholine receptor deficiency; Myasthenic Syndrome, Congenital, 17, 2A (slow-channel), 4B (fast-channel), and without tubular aggregates; Myeloperoxidase deficiency; MYH-associated polyposis; Endometrial carcinoma; Myocardial infarction 1; Myoclonic dystonia; Myoclonic-Atonic Epilepsy; Myoclonus with epilepsy with ragged red fibers; Myofibrillar myopathy 1 and ZASP-related; Myoglobinuria, acute recurrent, autosomal recessive; Myoneural gastrointestinal encephalopathy syndrome; Cerebellar ataxia infantile with progressive external ophthalmoplegia; Mitochondrial DNA depletion syndrome 4B, MNGIE type; Myopathy, centronuclear, 1, congenital, with excess of muscle spindles, distal, 1, lactic acidosis, and sideroblastic anemia 1, mitochondrial progressive with congenital cataract, hearing loss, and developmental delay, and tubular aggregate, 2; Myopia 6; Myosclerosis, autosomal recessive; Myotonia congenital; Congenital myotonia, autosomal dominant and recessive forms; Nail-patella syndrome; Nance-Horan syndrome; Nanophthalmos 2; Navajo neurohepatopathy; Nemaline myopathy 3 and 9; Neonatal hypotonia; Intellectual disability; Seizures; Delayed speech and language development; Mental retardation, autosomal dominant 31; Neonatal intrahepatic cholestasis caused by citrin deficiency; Nephrogenic diabetes insipidus, Nephrogenic diabetes insipidus, X-linked; Nephrolithiasis/osteoporosis, hypophosphatemic, 2; Nephronophthisis 13, 15 and 4; Infertility; Cerebello-oculo-renal syndrome (nephronophthisis, oculomotor apraxia and cerebellar abnormalities); Nephrotic syndrome, type 3, type 5, with or without ocular abnormalities, type 7, and type 9; Nestor-Guillermo progeria syndrome; Neu-Laxova syndrome 1; Neurodegeneration with brain iron accumulation 4 and 6; Neuroferritinopathy; Neurofibromatosis, type land type 2; Neurofibrosarcoma; Neurohypophyseal diabetes insipidus; Neuropathy, Hereditary Sensory, Type IC; Neutral 1 amino acid transport defect; Neutral lipid storage disease with myopathy; Neutrophil immunodeficiency syndrome; Nicolaides-Baraitser syndrome; Niemann-Pick disease type C1, C2, type A, and type C1, adult form; Non-ketotic hyperglycinemia; Noonan syndrome 1 and 4, LEOPARD syndrome 1; Noonan syndrome-like disorder with or without juvenile myelomonocytic leukemia; Normokalemic periodic paralysis, potassium-sensitive; Norum disease; Epilepsy, Hearing Loss, And Mental Retardation Syndrome; Mental Retardation, X-Linked 102 and syndromic 13; Obesity; Ocular albinism, type I; Oculocutaneous albinism type 1B, type 3, and type 4; Oculodentodigital dysplasia; Odontohypophosphatasia; Odontotrichomelic syndrome; Oguchi disease; Oligodontia-colorectal cancer syndrome; Opitz G/BBB syndrome; Optic atrophy 9; Oral-facial-digital syndrome; Ornithine aminotransferase deficiency; Orofacial cleft 11 and 7, Cleft lip/palate-ectodermal dysplasia syndrome; Orstavik Lindemann Solberg syndrome; Osteoarthritis with mild chondrodysplasia; Osteochondritis dissecans; Osteogenesis imperfecta type 12, type 5, type 7, type 8, type I, type III, with normal sclerae, dominant form, recessive perinatal lethal; Osteopathia striata with cranial sclerosis; Osteopetrosis autosomal dominant type 1 and 2, recessive 4, recessive 1, recessive 6; Osteoporosis with pseudoglioma; Oto-palato-digital syndrome, types I and II; Ovarian dysgenesis 1; Ovarioleukodystrophy; Pachyonychia congenita 4 and type 2; Paget disease of bone, familial; Pallister-Hall syndrome; Palmoplantar keratoderma, nonepidermolytic, focal or diffuse; Pancreatic agenesis and congenital heart disease; Papillon-Lef\xc3\xa8vre syndrome; Paragangliomas 3; Paramyotonia congenita of von Eulenburg; Parathyroid carcinoma; Parkinson disease 14, 15, 19 (juvenile-onset), 2, 20 (early-onset), 6, (autosomal recessive early-onset, and 9; Partial albinism; Partial hypoxanthine-guanine phosphoribosyltransferase deficiency; Patterned dystrophy of retinal pigment epithelium; PC-K6a; Pelizaeus-Merzbacher disease; Pendred syndrome; Peripheral demyelinating neuropathy, central dysmyelination; Hirschsprung disease; Permanent neonatal diabetes mellitus; Diabetes mellitus, permanent neonatal, with neurologic features; Neonatal insulin-dependent diabetes mellitus; Maturity-onset diabetes of the young, type 2; Peroxisome biogenesis disorder 14B, 2A, 4A, 5B, 6A, 7A, and 7B; Perrault syndrome 4; Perry syndrome; Persistent hyperinsulinemic hypoglycemia of infancy; familial hyperinsulinism; Phenotypes; Phenylketonuria; Pheochromocytoma; Hereditary Paraganglioma-Pheochromocytoma Syndromes; Paragangliomas 1; Carcinoid tumor of intestine; Cowden syndrome 3; Phosphoglycerate dehydrogenase deficiency; Phosphoglycerate kinase 1 deficiency; Photosensitive trichothiodystrophy; Phytanic acid storage disease; Pick disease; Pierson syndrome; Pigmentary retinal dystrophy; Pigmented nodular adrenocortical disease, primary, 1; Pilomatrixoma; Pitt-Hopkins syndrome; Pituitary dependent hypercortisolism; Pituitary hormone deficiency, combined 1, 2, 3, and 4; Plasminogen activator inhibitor type 1 deficiency; Plasminogen deficiency, type I; Platelet-type bleeding disorder 15 and 8; Poikiloderma, hereditary fibrosing, with tendon contractures, myopathy, and pulmonary fibrosis; Polycystic kidney disease 2, adult type, and infantile type; Polycystic lipomembranous osteodysplasia with sclerosing leukoencephalopathy; Polyglucosan body myopathy 1 with or without immunodeficiency; Polymicrogyria, asymmetric, bilateral frontoparietal; Polyneuropathy, hearing loss, ataxia, retinitis pigmentosa, and cataract; Pontocerebellar hypoplasia type 4; Popliteal pterygium syndrome; Porencephaly 2; Porokeratosis 8, disseminated superficial actinic type; Porphobilinogen synthase deficiency; porphyria cutanea tarda; Posterior column ataxia with retinitis pigmentosa; Posterior polar cataract type 2; Prader-Willi-like syndrome; Premature ovarian failure 4, 5, 7, and 9; Primary autosomal recessive microcephaly 10, 2, 3, and 5; Primary ciliary dyskinesia 24; Primary dilated cardiomyopathy; Left ventricular noncompaction 6; 4, Left ventricular noncompaction 10; Paroxysmal atrial fibrillation; Primary hyperoxaluria, type I, type, and type III; Primary hypertrophic osteoarthropathy, autosomal recessive 2; Primary hypomagnesemia; Primary open angle glaucoma juvenile onset 1; Primary pulmonary hypertension; Primrose syndrome; Progressive familial heart block type 1B; Progressive familial intrahepatic cholestasis 2 and 3; Progressive intrahepatic cholestasis; Progressive myoclonus epilepsy with ataxia; Progressive pseudorheumatoid dysplasia; Progressive sclerosing poliodystrophy; Prolidase deficiency; Proline dehydrogenase deficiency; Schizophrenia 4; Properdin deficiency, X-linked; Propionic academia; Proprotein convertase 1/3 deficiency; Prostate cancer, hereditary, 2; Protan defect; Proteinuria; Finnish congenital nephrotic syndrome; Proteus syndrome; Breast adenocarcinoma; Pseudoachondroplastic spondyloepiphyseal dysplasia syndrome; Pseudohypoaldosteronism type 1 autosomal dominant and recessive and type 2; Pseudohypoparathyroidism type 1A, Pseudopseudohypoparathyroidism; Pseudoneonatal adrenoleukodystrophy; Pseudoprimary hyperaldosteronism; Pseudoxanthoma elasticum; Generalized arterial calcification of infancy 2; Pseudoxanthoma elasticum-like disorder with multiple coagulation factor deficiency; Psoriasis susceptibility 2; PTEN hamartoma tumor syndrome; Pulmonary arterial hypertension related to hereditary hemorrhagic telangiectasia; Pulmonary Fibrosis And/Or Bone Marrow Failure, Telomere-Related, 1 and 3; Pulmonary hypertension, primary, 1, with hereditary hemorrhagic telangiectasia; Purine-nucleoside phosphorylase deficiency; Pyruvate carboxylase deficiency; Pyruvate dehydrogenase E1-alpha deficiency; Pyruvate kinase deficiency of red cells; Raine syndrome; Rasopathy; Recessive dystrophic epidermolysis bullosa; Nail disorder, nonsyndromic congenital, 8; Reifenstein syndrome; Renal adysplasia; Renal carnitine transport defect; Renal coloboma syndrome; Renal dysplasia; Renal dysplasia, retinal pigmentary dystrophy, cerebellar ataxia and skeletal dysplasia; Renal tubular acidosis, distal, autosomal recessive, with late-onset sensorineural hearing loss, or with hemolytic anemia; Renal tubular acidosis, proximal, with ocular abnormalities and mental retardation; Retinal cone dystrophy 3B; Retinitis pigmentosa; Retinitis pigmentosa 10, 11, 12, 14, 15, 17, and 19; Retinitis pigmentosa 2, 20, 25, 35, 36, 38, 39, 4, 40, 43, 45, 48, 66, 7, 70, 72; Retinoblastoma; Rett disorder; Rhabdoid tumor predisposition syndrome 2; Rhegmatogenous retinal detachment, autosomal dominant; Rhizomelic chondrodysplasia punctata type 2 and type 3; Roberts-SC phocomelia syndrome; Robinow Sorauf syndrome; Robinow syndrome, autosomal recessive, autosomal recessive, with brachy-syn-polydactyly; Rothmund-Thomson syndrome; Rapadilino syndrome; RRM2B-related mitochondrial disease; Rubinstein-Taybi syndrome; Salla disease; Sandhoff disease, adult and infantil types; Sarcoidosis, early-onset; Blau syndrome; Schindler disease, type 1; Schizencephaly; Schizophrenia 15; Schneckenbecken dysplasia; Schwannomatosis 2; Schwartz Jampel syndrome type 1; Sclerocornea, autosomal recessive; Sclerosteosis; Secondary hypothyroidism; Segawa syndrome, autosomal recessive; Senior-Loken syndrome 4 and 5, Sensory ataxic neuropathy, dysarthria, and ophthalmoparesis; Sepiapterin reductase deficiency; SeSAME syndrome; Severe combined immunodeficiency due to ADA deficiency, with microcephaly, growth retardation, and sensitivity to ionizing radiation, atypical, autosomal recessive, T cell-negative, B cell-positive, NK cell-negative of NK-positive; Partial adenosine deaminase deficiency; Severe congenital neutropenia; Severe congenital neutropenia 3, autosomal recessive or dominant; Severe congenital neutropenia and 6, autosomal recessive; Severe myoclonic epilepsy in infancy; Generalized epilepsy with febrile seizures plus, types 1 and 2; Severe X-linked myotubular myopathy; Short QT syndrome 3; Short stature with nonspecific skeletal abnormalities; Short stature, auditory canal atresia, mandibular hypoplasia, skeletal abnormalities; Short stature, onychodysplasia, facial dysmorphism, and hypotrichosis; Primordial dwarfism; Short-rib thoracic dysplasia 11 or 3 with or without polydactyly; Sialidosis type I and II; Silver spastic paraplegia syndrome; Slowed nerve conduction velocity, autosomal dominant; Smith-Lemli-Opitz syndrome; Snyder Robinson syndrome; Somatotroph adenoma; Prolactinoma; familial, Pituitary adenoma predisposition; Sotos syndrome 1 or 2; Spastic ataxia 5, autosomal recessive, Charlevoix-Saguenay type, 1, 10, or 11, autosomal recessive; Amyotrophic lateral sclerosis type 5; Spastic paraplegia 15, 2, 3, 35, 39, 4, autosomal dominant, 55, autosomal recessive, and 5A; Bile acid synthesis defect, congenital, 3; Spermatogenic failure 11, 3, and 8; Spherocytosis types 4 and 5; Spheroid body myopathy; Spinal muscular atrophy, lower extremity predominant 2, autosomal dominant; Spinal muscular atrophy, type II; Spinocerebellar ataxia 14, 21, 35, 40, and 6; Spinocerebellar ataxia autosomal recessive 1 and 16; Splenic hypoplasia; Spondylocarpotarsal synostosis syndrome; Spondylocheirodysplasia, Ehlers-Danlos syndrome-like, with immune dysregulation, Aggrecan type, with congenital joint dislocations, short limb-hand type, Sedaghatian type, with cone-rod dystrophy, and Kozlowski type; Parastremmatic dwarfism; Stargardt disease 1; Cone-rod dystrophy 3; Stickler syndrome type 1; Kniest dysplasia; Stickler syndrome, types 1(nonsyndromic ocular) and 4; Sting-associated vasculopathy, infantile-onset; Stormorken syndrome; Sturge-Weber syndrome, Capillary malformations, congenital, 1; Succinyl-CoA acetoacetate transferase deficiency; Sucrase-isomaltase deficiency; Sudden infant death syndrome; Sulfite oxidase deficiency, isolated; Supravalvar aortic stenosis; Surfactant metabolism dysfunction, pulmonary, 2 and 3; Symphalangism, proximal, lb; Syndactyly Cenani Lenz type; Syndactyly type 3; Syndromic X-linked mental retardation 16; Talipes equinovarus; Tangier disease; TARP syndrome; Tay-Sachs disease, B1 variant, Gm2-gangliosidosis (adult), Gm2-gangliosidosis (adult-onset); Temtamy syndrome; Tenorio Syndrome; Terminal osseous dysplasia; Testosterone 17-beta-dehydrogenase deficiency; Tetraamelia, autosomal recessive; Tetralogy of Fallot; Hypoplastic left heart syndrome 2; Truncus arteriosus; Malformation of the heart and great vessels; Ventricular septal defect 1; Thiel-Behnke corneal dystrophy; Thoracic aortic aneurysms and aortic dissections; Marfanoid habitus; Three M syndrome 2; Thrombocytopenia, platelet dysfunction, hemolysis, and imbalanced globin synthesis; Thrombocytopenia, X-linked; Thrombophilia, hereditary, due to protein C deficiency, autosomal dominant and recessive; Thyroid agenesis; Thyroid cancer, follicular; Thyroid hormone metabolism, abnormal; Thyroid hormone resistance, generalized, autosomal dominant; Thyrotoxic periodic paralysis and Thyrotoxic periodic paralysis 2; Thyrotropin-releasing hormone resistance, generalized; Timothy syndrome; TNF receptor-associated periodic fever syndrome (TRAPS); Tooth agenesis, selective, 3 and 4; Torsades de pointes; Townes-Brocks-branchiootorenal-like syndrome; Transient bullous dermolysis of the newborn; Treacher collins syndrome 1; Trichomegaly with mental retardation, dwarfism and pigmentary degeneration of retina; Trichorhinophalangeal dysplasia type I; Trichorhinophalangeal syndrome type 3; Trimethylaminuria; Tuberous sclerosis syndrome; Lymphangiomyomatosis; Tuberous sclerosis 1 and 2; Tyrosinase-negative oculocutaneous albinism; Tyrosinase-positive oculocutaneous albinism; Tyrosinemia type I; UDPglucose-4-epimerase deficiency; Ullrich congenital muscular dystrophy; Ulna and fibula absence of with severe limb deficiency; Upshaw-Schulman syndrome; Urocanate hydratase deficiency; Usher syndrome, types 1, 1B, 1D, 1G, 2A, 2C, and 2D; Retinitis pigmentosa 39; UV-sensitive syndrome; Van der Woude syndrome; Van Maldergem syndrome 2; Hennekam lymphangiectasia-lymphedema syndrome 2; Variegate porphyria; Ventriculomegaly with cystic kidney disease; Verheij syndrome; Very long chain acyl-CoA dehydrogenase deficiency; Vesicoureteral reflux 8; Visceral heterotaxy 5, autosomal; Visceral myopathy; Vitamin D-dependent rickets, types land 2; Vitelliform dystrophy; von Willebrand disease type 2M and type 3; Waardenburg syndrome type 1, 4C, and 2E (with neurologic involvement); Klein-Waardenberg syndrome; Walker-Warburg congenital muscular dystrophy; Warburg micro syndrome 2 and 4; Warts, hypogammaglobulinemia, infections, and myelokathexis; Weaver syndrome; Weill-Marchesani syndrome 1 and 3; Weill-Marchesani-like syndrome; Weissenbacher-Zweymuller syndrome; Werdnig-Hoffmann disease; Charcot-Marie-Tooth disease; Werner syndrome; WFS1-Related Disorders; Wiedemann-Steiner syndrome; Wilson disease; Wolfram-like syndrome, autosomal dominant; Worth disease; Van Buchem disease type 2; Xeroderma pigmentosum, complementation group b, group D, group E, and group G; X-linked agammaglobulinemia; X-linked hereditary motor and sensory neuropathy; X-linked ichthyosis with steryl-sulfatase deficiency; X-linked periventricular heterotopia; Oto-palato-digital syndrome, type I; X-linked severe combined immunodeficiency; Zimmermann-Laband syndrome and Zimmermann-Laband syndrome 2; and Zonular pulverulent cataract 3.

In some aspects, the present disclosure provides uses of any one of the base editors described herein and a guide RNA targeting this base editor to a target A:T base pair in a nucleic acid molecule in the manufacture of a kit for nucleic acid editing, wherein the nucleic acid editing comprises contacting the nucleic acid molecule with the base editor and guide RNA under conditions suitable for the substitution of the adenine (A) of the A:T nucleobase pair with an guanine (G). In some embodiments of these uses, the nucleic acid molecule is a double-stranded DNA molecule. In some embodiments, the step of contacting of induces separation of the double-stranded DNA at a target region. In some embodiments, the step of contacting further comprises nicking one strand of the double-stranded DNA, wherein the one strand comprises an unmutated strand that comprises the T of the target A:T nucleobase pair.

In some aspects, the present disclosure provides uses of any one of the base editors described herein and a guide RNA targeting this base editor to a target A:T base pair in a nucleic acid molecule in the manufacture of a kit for evaluating the off-target effects of a base editor, wherein the step of evaluating the off-target effects comprises contacting the base editor with the nucleic acid molecule and determining off-target effects in accordance with any one of the disclosed methods. In some embodiments of these uses, the nucleic acid molecule is a double-stranded DNA molecule. In some embodiments, the step of contacting of induces separation of the double-stranded DNA at a target region. In some embodiments, the step of contacting further comprises nicking one strand of the double-stranded DNA, wherein the one strand comprises an unmutated strand that comprises the T of the target A:T nucleobase pair.

In some embodiments of the described uses, the step of contacting is performed in vitro. In other embodiments, the step of contacting is performed in vivo. In some embodiments, the step of contacting is performed in a subject (e.g., a human subject or a non-human animal subject). In some embodiments, the step of contacting is performed in a cell, such as a human or non-human animal cell.

The present disclosure also provides uses of any one of the base editors described herein as a medicament. The present disclosure also provides uses of any one of the complexes of base editors and guide RNAs described herein as a medicament. Some aspects of this disclosure provide methods of using the fusion proteins, or complexes comprising a guide nucleic acid (e.g., gRNA) and a nucleobase editor provided herein to edit DNA, e.g., to edit SMN2. For example, some aspects of this disclosure provide methods comprising contacting a DNA, or RNA molecule with any of the fusion proteins provided herein, and with at least one guide nucleic acid (e.g., guide RNA), wherein the guide nucleic acid, (e.g., guide RNA) is comprises a sequence (e.g., a guide sequence that binds to a DNA target sequence) of at least 10 (e.g., at least 10, 15, 20, 25, or 30) contiguous nucleotides that is 100% complementary to a target sequence (e.g., any of the target SMN2 sequences provided herein). In some embodiments, the 3′ end of the target sequence is immediately adjacent to a canonical PAM sequence (NGG). In some embodiments, the 3′ end of the target sequence is not immediately adjacent to a canonical PAM sequence (NGG). In some embodiments, the 3′ end of the target sequence is immediately adjacent to an AGC, GAG, TTT, GTG, or CAA sequence.

Some aspects of the disclosure provide methods of using base editors (e.g., any of the fusion proteins provided herein) and gRNAs to correct a point mutation in an SMN2 gene. In some embodiments, the disclosure provides methods of using base editors (e.g., any of the fusion proteins provided herein) and gRNAs to generate an A to G and/or T to C mutation in an SMN2 gene. In some embodiments, the disclosure provides method for deaminating an adenosine nucleobase (A) in an SMN2 gene, the method comprising contacting the SMN2 gene with a base editor and a guide RNA bound to the base editor, where the guide RNA comprises a guide sequence that is complementary to a target nucleic acid sequence in the SMN2 gene. In some embodiments, the SMN2 gene comprises a C to T mutation. In some embodiments, the C to T mutation in the SMN2 gene masks an acceptor splice site, resulting in a truncated SMN protein encoded by the SMN2 gene (i.e., exon 7 is not transcribed). While the resulting protein functions as a full-length SMN protein, it is prone to rapid degradation due to the presence of an EMLA tail from exon 8 and the exposed exon 6 C-terminal amino acid chain. In some embodiments, the C to T mutation in the SMN2 gene results in the degradation of at least 1%, 2%, 5%, 10%, 15%, 20%, 25%, 30%, 35%, 40%, 45%, 50%, 55%, 60%, 65%, 70%, 75%, 80%, 85%, 90%, 95%, 98%, or at least 99% of the resulting SMN protein.

In some embodiments, deaminating an adenosine (A) nucleobase complementary to the T corrects the C to T mutation in the SMN2 gene. In some embodiments, the C to T or G to A mutation in the SMN2 gene leads to a Cys (C) to Tyr (Y) mutation in the SMN2 protein encoded by the SMN2 gene. In some embodiments, deaminating the adenosine nucleobase complementary to the T corrects the Cys to Tyr mutation in the SMN2 protein.

In some embodiments, the guide sequence of the gRNA comprises at least 8, 9, 10, 11, 12, 13, 14, 15, 16, 17, 18, 19, 20, 21, 22, 23, 24, 25, 26, 27, 28, 29, 30, 31, 32, 33, 34, or 35 contiguous nucleic acids that are 100% complementary to a target nucleic acid sequence of the SMN2 gene. In some embodiments, the base editor nicks the target sequence that is complementary to the guide sequence.

In some embodiments, the target DNA sequence comprises a sequence associated with a disease or disorder, e.g., SMA. In some embodiments, the target DNA sequence comprises a point mutation associated with a disease or disorder (e.g., exon 7 of SMN2). In some embodiments, the activity of the fusion protein (e.g., comprising an adenosine deaminase and a Cas9 domain), or the complex, results in a correction of the point mutation. In some embodiments, the target DNA sequence comprises a C→T point mutation associated with a disease or disorder, and wherein the deamination of the mutant base results in a sequence that is not associated with a disease or disorder. In some embodiments, the target DNA sequence encodes a protein, and the point mutation is in a codon and results in a change in the splice site of an exon, resulting in production of a full-length, fully functional protein (e.g., SMN protein). In some embodiments, the deamination of the mutant base results in the wild-type amino acid.

In some embodiments, the target DNA sequence comprises a sequence associated with a stop codon in an exon 8 of a SMN2 gene. In some embodiments, the activity of the fusion protein (e.g., comprising an adenosine deaminase and a Cas9 domain), or the complex, results in destruction of the stop codon and/or a frameshift mutation. Without wishing to be bound by theory, it is thought that destroying a stop codon (e.g., the 5^{th} codon stop sequence) and/or inducing at least one frameshift mutation result in a more stable SMN protein product, regardless of whether amino acids encoded by exon 7 are included in the protein. For example, in one embodiment, activity of the fusion protein (e.g., comprising an adenosine deaminase and a Cas9 domain), or the complex, results in adenine deamination of the 5^{th} codon stop sequence of a SMN2's exon 8, facilitating the addition of five amino acids at the C-terminal end of the translated SMN protein.

In some embodiments, the target DNA sequence comprises a sequence associated with an amino acid present in exon 6 of an SMN2 gene. Modification of one amino acid (e.g., 5270) using the methods described herein, can be used to slow the rate of SMN protein degradation.

In some embodiments, the contacting is in vivo in a subject. In some embodiments, the subject has or has been diagnosed with a disease or disorder (e.g., SMA).

Some embodiments provide methods for using the DNA editing fusion proteins provided herein. In some embodiments, the fusion protein is used to introduce a point mutation into a nucleic acid by deaminating a target nucleobase. In some embodiments, the deamination of the target nucleobase results in the correction of a genetic defect, e.g., in the correction of a point mutation that leads to degradation of the resulting SMN protein. In some embodiments, the genetic defect is associated with a disease or disorder, e.g., SMA.

In some embodiments, the purposes of the methods provided herein are to restore the full-length gene or to stabilize the resulting protein product via genome editing. The nucleobase editing proteins provided herein can be validated for gene editing-based human therapeutics in vitro, e.g., by correcting a disease-associated mutation in human cell culture. It will be understood by the skilled artisan that the nucleobase editing proteins provided herein, e.g., the fusion proteins comprising a nucleic acid programmable DNA binding protein (e.g., Cas9) and an adenosine deaminase domain can be used to correct any single point G to A or C to T mutation. In the first case, deamination of the mutant A to I corrects the mutation, and in the latter case, deamination of the A that is base-paired with the mutant T, followed by a round of replication or followed by base editing repair activity, corrects the mutation.

The instant disclosure provides methods for the treatment of a subject diagnosed with a disease associated with or caused by a point mutation that can be corrected by a DNA editing fusion protein provided herein. For example, in some embodiments, a method is provided that comprises administering to a subject having such a disease, e.g., SMA.

In some embodiments, a fusion protein recognizes canonical PAMs and therefore can correct the pathogenic G to A or C to T mutations with canonical PAMs, e.g., NGG, respectively, in the flanking sequences. For example, Cas9 proteins that recognize canonical PAMs comprise an amino acid sequence that is at least 80%, 85%, 90%, 95%, 97%, 98%, or 99% identical to the amino acid sequence of Streptococcus pyogenes Cas9 as provided by any one of SEQ ID NOs: 5, 8, 10, 12, and 407 or to a fragment thereof comprising the RuvC and HNH domains of any one of SEQ ID NOs: 5, 8, 10, 12, and 407.

It will be apparent to those of skill in the art that in order to target any of the fusion proteins comprising a Cas9 domain and an adenosine deaminase, as disclosed herein, to a target site, e.g., a site comprising a point mutation to be edited, it is typically necessary to co-express the fusion protein together with a guide RNA, e.g., an sgRNA. As explained in more detail elsewhere herein, a guide RNA typically comprises a tracrRNA framework allowing for Cas9 binding, and a guide sequence, which confers sequence specificity to the Cas9:nucleic acid editing enzyme/domain fusion protein. In some embodiments, the guide RNA sequence 5′-ATTTTGTCTAAAACCCTGTA-3′ (SEQ ID NO: 331), where the nucleotide target is indicated in bold. It should be appreciated that the Ts indicated in the gRNA sequence are uracil (Us) in the RNA sequence. Accordingly, in some embodiments, the gRNA comprises the sequence 5′-AUUUUGUCUAAAACCCUGUA-3′ (SEQ ID NO: 332).

In some embodiments, the guide sequence of the gRNA comprises a nucleic acid sequence selected from the group consisting of 5′-TTTGTCTAAAACCCTGTAAG-3′ (SEQ ID NO: 333), 5′-TTTTGTCTAAAACCCTGTAA-3′ (SEQ ID NO: 334), 5′-TGATTTTGTCTAAAACCC-3′ (SEQ ID NO: 335), 5′-GATTTTGTCTAAAACCCT-3′ (SEQ ID NO: 336), 5′-ATTTTGTCTAAAACCCTG-3′ (SEQ ID NO: 337), 5′-GTCTAAAACCCTGTAAGG-3′ (SEQ ID NO: 338), and 5′-TCTAAAACCCTGTAAGGA-3′ (SEQ ID NO: 339). As noted previously, the gRNA sequence may comprise uracil (U) instead of thymine (T). Therefore, in some embodiments, the guide sequence of the gRNA comprises a nucleic acid sequence selected from the group consisting of 5′-UUUGUCUAAAACCCUGUAAG-3′ (SEQ ID NO: 340), 5′-UUUUGUCUAAAACCCUGUAA-3′ (SEQ ID NO: 341), 5′-UGAUUUUGUCUAAAACCC-3′ (SEQ ID NO: 342), 5′-GAUUUUGUCUAAAACCCU-3′ (SEQ ID NO: 343), 5′-AUUUUGUCUAAAACCCUG-3′ (SEQ ID NO: 344), 5′-GUCUAAAACCCUGUAAGG-3′ (SEQ ID NO: 345), and 5′-UCUAAAACCCUGUAAGGA-3′ (SEQ ID NO: 346).

In some embodiments, the gRNA comprises a nucleic acid sequence selected from the group consisting of: 5′-TTTGCAGGAAATGCTGGCAT-3′ (SEQ ID NO: 347), 5′-TTCTCATTTGCAGGAAATGC-3′ (SEQ ID NO: 348), 5′-CATTTAGTGCTGCTCTATGC-3′ (SEQ ID NO: 349), 5′-CAGGAAATGCTGGCATAGAG-3′ (SEQ ID NO: 350), 5′-TTGCAGGAAATGCTGGCATA-3′ (SEQ ID NO: 351), 5′-ATTTGCAGGAAATGCTGGCA-3′ (SEQ ID NO: 352), and 5′-TGGCATAGAGCAGCACTAAA-3′ (SEQ ID NO: 353), where the nucleotide target is indicated in bold. It should be appreciated that the Ts indicated in the gRNA sequence are uracil (Us) in the RNA sequence. Accordingly, in some embodiments, the gRNA comprises a sequence selected from the group consisting of: 5′-UUUGCAGGAAAUGCUGGCAU-3′ (SEQ ID NO: 354), 5′-UUCUCAUUUGCAGGAAAUGC-3′ (SEQ ID NO: 355), 5′-CAUUUAGUGCUGCUCUAUGC-3′ (SEQ ID NO: 356), 5′-CAGGAAAUGCUGGCAUAGAG-3′ (SEQ ID NO: 357), 5′-UUGCAGGAAAUGCUGGCAUA-3′ (SEQ ID NO: 358), 5′-AUUUGCAGGAAAUGCUGGCA-3′ (SEQ ID NO: 359), and 5′-UGGCAUAGAGCAGCACUAAA-3′ (SEQ ID NO: 360).

In some embodiments, the gRNA comprises the nucleic acid sequence 5′-TGGCATAGAGCAGCACTAAA-3′ (SEQ ID NO: 361), where the nucleotide target is indicated in bold. It should be appreciated that the Ts indicated in the gRNA sequence are uracil (Us) in the RNA sequence. Accordingly, in some embodiments, the gRNA comprises the sequence: 5′-UGGCAUAGAGCAGCACUAAA-3′ (SEQ ID NO: 362).

Some aspects of the disclosure provide methods for editing a nucleic acid. In some embodiments, the method is a method for editing a nucleobase of a nucleic acid (e.g., a base pair of a double-stranded DNA sequence). In some embodiments, the method comprises the steps of: a) contacting a target region of a nucleic acid (e.g., a double-stranded DNA sequence) with a complex comprising a base editor (e.g., a Cas9 domain fused to an adenosine deaminase) and a guide nucleic acid (e.g., gRNA), wherein the target region comprises a targeted nucleobase pair, b) inducing strand separation of said target region, c) converting a first nucleobase of said target nucleobase pair in a single strand of the target region to a second nucleobase, and d) cutting no more than one strand of said target region, where a third nucleobase complementary to the first nucleobase base is replaced by a fourth nucleobase complementary to the second nucleobase. In some embodiments, the method results in less than 20% indel formation in the nucleic acid. It should be appreciated that in some embodiments, step b is omitted. In some embodiments, the first nucleobase is an adenine. In some embodiments, the second nucleobase is a deaminated adenine, or inosine. In some embodiments, the third nucleobase is a thymine. In some embodiments, the fourth nucleobase is a cytosine. In some embodiments, the method results in less than 19%, 18%, 16%, 14%, 12%, 10%, 8%, 6%, 4%, 2%, 1%, 0.5%, 0.2%, or less than 0.1% indel formation. In some embodiments, the method further comprises replacing the second nucleobase with a fifth nucleobase that is complementary to the fourth nucleobase, thereby generating an intended edited base pair (e.g., A:T to G:C). In some embodiments, the fifth nucleobase is a guanine. In some embodiments, at least 5% of the intended base pairs are edited. In some embodiments, at least 10%, 15%, 20%, 25%, 30%, 35%, 40%, 45%, or 50% of the intended base pairs are edited.

In some embodiments, the ratio of intended products to unintended products in the target nucleotide is at least 2:1, 5:1, 10:1, 20:1, 30:1, 40:1, 50:1, 60:1, 70:1, 80:1, 90:1, 100:1, or 200:1, or more. In some embodiments, the ratio of intended point mutation to indel formation is greater than 1:1, 10:1, 50:1, 100:1, 500:1, or 1000:1, or more. In some embodiments, the cut single strand (nicked strand) is hybridized to the guide nucleic acid. In some embodiments, the cut single strand is opposite to the strand comprising the first nucleobase. In some embodiments, the base editor comprises a Cas9 domain. In some embodiments, the first base is adenine, and the second base is not a G, C, A, or T. In some embodiments, the second base is inosine. In some embodiments, the first base is adenine. In some embodiments, the second base is not a G, C, A, or T. In some embodiments, the second base is inosine. In some embodiments, the base editor inhibits base excision repair of the edited strand. In some embodiments, the base editor protects or binds the non-edited strand. In some embodiments, the base editor comprises UGI activity. In some embodiments, the base editor comprises a catalytically inactive inosine-specific nuclease. In some embodiments, the base editor comprises nickase activity. In some embodiments, the intended edited base pair is upstream of a PAM site. In some embodiments, the intended edited base pair is 1, 2, 3, 4, 5, 6, 7, 8, 9, 10, 11, 12, 13, 14, 15, 16, 17, 18, 19, or 20 nucleotides upstream of the PAM site. In some embodiments, the intended edited base pair is downstream of a PAM site. In some embodiments, the intended edited base pair is 1, 2, 3, 4, 5, 6, 7, 8, 9, 10, 11, 12, 13, 14, 15, 16, 17, 18, 19, or 20 nucleotides downstream stream of the PAM site. In some embodiments, the method does not require a canonical (e.g., NGG) PAM site. In some embodiments, the nucleobase editor comprises a linker. In some embodiments, the linker is 1-25 amino acids in length. In some embodiments, the linker is 5-20 amino acids in length. In some embodiments, linker is 10, 11, 12, 13, 14, 15, 16, 17, 18, 19, or 20 amino acids in length. In some embodiments, the target region comprises a target window, wherein the target window comprises the target nucleobase pair. In some embodiments, the target window comprises 1-10 nucleotides. In some embodiments, the target window is 1-9, 1-8, 1-7, 1-6, 1-5, 1-4, 1-3, 1-2, or 1 nucleotides in length. In some embodiments, the target window is 1, 2, 3, 4, 5, 6, 7, 8, 9, 10, 11, 12, 13, 14, 15, 16, 17, 18, 19, or 20 nucleotides in length. In some embodiments, the intended edited base pair is within the target window. In some embodiments, the target window comprises the intended edited base pair. In some embodiments, the method is performed using any of the base editors provided herein. In some embodiments, a target window is a deamination window.

In some embodiments, the disclosure provides methods for editing a nucleotide. In some embodiments, the disclosure provides a method for editing a nucleobase pair of a double-stranded DNA sequence. In some embodiments, the method comprises a) contacting a target region of the double-stranded DNA sequence with a complex comprising a base editor and a guide nucleic acid (e.g., gRNA), where the target region comprises a target nucleobase pair, b) inducing strand separation of said target region, c) converting a first nucleobase of said target nucleobase pair in a single strand of the target region to a second nucleobase, d) cutting no more than one strand of said target region, wherein a third nucleobase complementary to the first nucleobase base is replaced by a fourth nucleobase complementary to the second nucleobase, and the second nucleobase is replaced with a fifth nucleobase that is complementary to the fourth nucleobase, thereby generating an intended edited base pair, wherein the efficiency of generating the intended edited base pair is at least 5%. It should be appreciated that in some embodiments, step b is omitted. In some embodiments, at least 5% of the intended base pairs are edited. In some embodiments, at least 10%, 15%, 20%, 25%, 30%, 35%, 40%, 45%, or 50% of the intended base pairs are edited. In some embodiments, the method causes less than 19%, 18%, 16%, 14%, 12%, 10%, 8%, 6%, 4%, 2%, 1%, 0.5%, 0.2%, or less than 0.1% indel formation. In some embodiments, the ratio of intended product to unintended products at the target nucleotide is at least 2:1, 5:1, 10:1, 20:1, 30:1, 40:1, 50:1, 60:1, 70:1, 80:1, 90:1, 100:1, or 200:1, or more. In some embodiments, the ratio of intended point mutation to indel formation is greater than 1:1, 10:1, 50:1, 100:1, 500:1, or 1000:1, or more. In some embodiments, the cut single strand is hybridized to the guide nucleic acid. In some embodiments, the cut single strand is opposite to the strand comprising the first nucleobase. In some embodiments, the first base is adenine. In some embodiments, the second nucleobase is not G, C, A, or T. In some embodiments, the second base is inosine. In some embodiments, the base editor inhibits base excision repair of the edited strand. In some embodiments, the base editor protects (e.g., form base excision repair) or binds the non-edited strand. In some embodiments, the nucleobase editor comprises UGI activity. In some embodiments, the base editor comprises a catalytically inactive inosine-specific nuclease. In some embodiments, the nucleobase editor comprises nickase activity. In some embodiments, the intended edited base pair is upstream of a PAM site. In some embodiments, the intended edited base pair is 1, 2, 3, 4, 5, 6, 7, 8, 9, 10, 11, 12, 13, 14, 15, 16, 17, 18, 19, or 20 nucleotides upstream of the PAM site. In some embodiments, the intended edited base pair is downstream of a PAM site. In some embodiments, the intended edited base pair is 1, 2, 3, 4, 5, 6, 7, 8, 9, 10, 11, 12, 13, 14, 15, 16, 17, 18, 19, or 20 nucleotides downstream stream of the PAM site. In some embodiments, the method does not require a canonical (e.g., NGG) PAM site. In some embodiments, the nucleobase editor comprises a linker. In some embodiments, the linker is 1-25 amino acids in length. In some embodiments, the linker is 5-20 amino acids in length. In some embodiments, the linker is 10, 11, 12, 13, 14, 15, 16, 17, 18, 19, or 20 amino acids in length. In some embodiments, the target region comprises a target window, wherein the target window comprises the target nucleobase pair. In some embodiments, the target window comprises 1-10 nucleotides. In some embodiments, the target window is 1-9, 1-8, 1-7, 1-6, 1-5, 1-4, 1-3, 1-2, or 1 nucleotides in length. In some embodiments, the target window is 1, 2, 3, 4, 5, 6, 7, 8, 9, 10, 11, 12, 13, 14, 15, 16, 17, 18, 19, or 20 nucleotides in length. In some embodiments, the intended edited base pair occurs within the target window. In some embodiments, the target window comprises the intended edited base pair. In some embodiments, the nucleobase editor is any one of the base editors provided herein.

The instant disclosure provides methods for the treatment of a subject diagnosed with a disease associated with or caused by a point mutation that can be corrected by the editing system provided herein, e.g., spinal muscular atrophy (SMA). For example, in some embodiments, a method is provided that comprises administering to a subject having such a disease, e.g., SMA, a an effective amount of the adenosine base editor and guide RNA described herein that corrects the exon 7 point mutation of SMN2 (e.g., the C840T mutation) or modifies a flanking exon (e.g., exon 6 or exon 8) so that the resulting SMN protein product more stable (e.g., is less prone to degradation).

X. Base Editor Delivery

In another aspect, the present disclosure provides for the delivery of base editors in vitro and in vivo using various strategies, including on separate vectors using split inteins and as well as direct delivery strategies of the ribonucleoprotein complex (i.e., the base editor complexed to the gRNA and/or the second-site gRNA) using techniques such as electroporation, use of cationic lipid-mediated formulations, and induced endocytosis methods using receptor ligands fused to to the ribonucleoprotein complexes. Any such methods are contemplated herein.

In some aspects, the invention provides methods comprising delivering one or more base editor-encoding polynucleotides, such as or one or more vectors as described herein encoding one or more components of the base editing system described herein, one or more transcripts thereof, and/or one or proteins transcribed therefrom, to a host cell. In some aspects, the invention further provides cells produced by such methods, and organisms (such as animals, plants, or fungi) comprising or produced from such cells. In some embodiments, a base editor as described herein in combination with (and optionally complexed with) a guide sequence is delivered to a cell. Conventional viral and non-viral based gene transfer methods can be used to introduce nucleic acids in mammalian cells or target tissues. Such methods can be used to administer nucleic acids encoding components of a base editor to cells in culture, or in a host organism. Non-viral vector delivery systems include DNA plasmids, RNA (e.g. a transcript of a vector described herein), naked nucleic acid, and nucleic acid complexed with a delivery vehicle, such as a liposome. Viral vector delivery systems include DNA and RNA viruses, which have either episomal or integrated genomes after delivery to the cell. For a review of gene therapy procedures, see Anderson, Science 256:808-813 (1992); Nabel & Felgner, TIBTECH 11:211-217 (1993); Mitani & Caskey, TIBTECH 11:162-166 (1993); Dillon, TIBTECH 11:167-175 (1993); Miller, Nature 357:455-460 (1992); Van Brunt, Biotechnology 6(10):1149-1154 (1988); Vigne, Restorative Neurology and Neuroscience 8:35-36 (1995); Kremer & Perricaudet, British Medical Bulletin 51(1):31-44 (1995); Haddada et al., in Current Topics in Microbiology and Immunology Doerfler and Bihm (eds) (1995); and Yu et al., Gene Therapy 1:13-26 (1994).

Methods of non-viral delivery of nucleic acids include lipofection, nucleofection, microinjection, biolistics, virosomes, liposomes, immunoliposomes, polycation or lipid:nucleic acid conjugates, naked DNA, artificial virions, and agent-enhanced uptake of DNA. Lipofection is described in e.g., U.S. Pat. Nos. 5,049,386, 4,946,787; and 4,897,355) and lipofection reagents are sold commercially (e.g., Transfectam™ and Lipofectin™) Cationic and neutral lipids that are suitable for efficient receptor-recognition lipofection of polynucleotides include those of Feigner, WO 91/17424; WO 91/16024. Delivery can be to cells (e.g. in vitro or ex vivo administration) or target tissues (e.g. in vivo administration).

The preparation of lipid:nucleic acid complexes, including targeted liposomes such as immunolipid complexes, is well known to one of skill in the art (see, e.g., Crystal, Science 270:404-410 (1995); Blaese et al., Cancer Gene Ther. 2:291-297 (1995); Behr et al., Bioconjugate Chem. 5:382-389 (1994); Remy et al., Bioconjugate Chem. 5:647-654 (1994); Gao et al., Gene Therapy 2:710-722 (1995); Ahmad et al., Cancer Res. 52:4817-4820 (1992); U.S. Pat. Nos. 4,186,183, 4,217,344, 4,235,871, 4,261,975, 4,485,054, 4,501,728, 4,774,085, 4,837,028, and 4,946,787).

The use of RNA or DNA viral based systems for the delivery of nucleic acids take advantage of highly evolved processes for targeting a virus to specific cells in the body and trafficking the viral payload to the nucleus. Viral vectors can be administered directly to patients (in vivo) or they can be used to treat cells in vitro, and the modified cells may optionally be administered to patients (ex vivo). Conventional viral based systems could include retroviral, lentivirus, adenoviral, adeno-associated and herpes simplex virus vectors for gene transfer. Integration in the host genome is possible with the retrovirus, lentivirus, and adeno-associated virus gene transfer methods, often resulting in long term expression of the inserted transgene. Additionally, high transduction efficiencies have been observed in many different cell types and target tissues.

The tropism of a viruses can be altered by incorporating foreign envelope proteins, expanding the potential target population of target cells. Lentiviral vectors are retroviral vectors that are able to transduce or infect non-dividing cells and typically produce high viral titers. Selection of a retroviral gene transfer system would therefore depend on the target tissue. Retroviral vectors are comprised of cis-acting long terminal repeats with packaging capacity for up to 6-10 kb of foreign sequence. The minimum cis-acting LTRs are sufficient for replication and packaging of the vectors, which are then used to integrate the therapeutic gene into the target cell to provide permanent transgene expression. Widely used retroviral vectors include those based upon murine leukemia virus (MuLV), gibbon ape leukemia virus (GaLV), Simian Immuno deficiency virus (SIV), human immuno deficiency virus (HIV), and combinations thereof (see, e.g., Buchscher et al., J. Virol. 66:2731-2739 (1992); Johann et al., J. Virol. 66:1635-1640 (1992); Sommnerfelt et al., Virol. 176:58-59 (1990); Wilson et al., J. Virol. 63:2374-2378 (1989); Miller et al., J. Virol. 65:2220-2224 (1991); PCT/US94/05700). In applications where transient expression is preferred, adenoviral based systems may be used. Adenoviral based vectors are capable of very high transduction efficiency in many cell types and do not require cell division. With such vectors, high titer and levels of expression have been obtained. This vector can be produced in large quantities in a relatively simple system. Adeno-associated virus (“AAV”) vectors may also be used to transduce cells with target nucleic acids, e.g., in the in vitro production of nucleic acids and peptides, and for in vivo and ex vivo gene therapy procedures (see, e.g., West et al., Virology 160:38-47 (1987); U.S. Pat. No. 4,797,368; WO 93/24641; Kotin, Human Gene Therapy 5:793-801 (1994); Muzyczka, J. Clin. Invest. 94:1351 (1994). Construction of recombinant AAV vectors are described in a number of publications, including U.S. Pat. No. 5,173,414; Tratschin et al., Mol. Cell. Biol. 5:3251-3260 (1985); Tratschin, et al., Mol. Cell. Biol. 4:2072-2081 (1984); Hermonat & Muzyczka, PNAS 81:6466-6470 (1984); and Samulski et al., J. Virol. 63:03822-3828 (1989).

Packaging cells are typically used to form virus particles that are capable of infecting a host cell. Such cells include 293 cells, which package adenovirus, and W2 cells or PA317 cells, which package retrovirus. Viral vectors used in gene therapy are usually generated by producing a cell line that packages a nucleic acid vector into a viral particle. The vectors typically contain the minimal viral sequences required for packaging and subsequent integration into a host, other viral sequences being replaced by an expression cassette for the polynucleotide(s) to be expressed. The missing viral functions are typically supplied in trans by the packaging cell line. For example, AAV vectors used in gene therapy typically only possess ITR sequences from the AAV genome which are required for packaging and integration into the host genome. Viral DNA is packaged in a cell line, which contains a helper plasmid encoding the other AAV genes, namely rep and cap, but lacking ITR sequences. The cell line may also be infected with adenovirus as a helper. The helper virus promotes replication of the AAV vector and expression of AAV genes from the helper plasmid. The helper plasmid is not packaged in significant amounts due to a lack of ITR sequences. Contamination with adenovirus can be reduced by, e.g., heat treatment to which adenovirus is more sensitive than AAV. Additional methods for the delivery of nucleic acids to cells are known to those skilled in the art. See, for example, US20030087817, incorporated herein by reference.

In various embodiments, the base editor constructs (including, the split-constructs) may be engineered for delivery in one or more rAAV vectors. An rAAV as related to any of the methods and compositions provided herein may be of any serotype including any derivative or pseudotype (e.g., 1, 2, 3, 4, 5, 6, 7, 8, 9, 10, 11, 12, 13, 2/1, 2/5, 2/8, 2/9, 3/1, 3/5, 3/8, or 3/9). An rAAV may comprise a genetic load (i.e., a recombinant nucleic acid vector that expresses a gene of interest, such as a whole or split base editor fusion protein that is carried by the rAAV into a cell) that is to be delivered to a cell. An rAAV may be chimeric.

As used herein, the serotype of an rAAV refers to the serotype of the capsid proteins of the recombinant virus. Non-limiting examples of derivatives and pseudotypes include rAAV2/1, rAAV2/5, rAAV2/8, rAAV2/9, AAV2-AAV3 hybrid, AAVrh.10, AAVhu.14, AAV3a/3b, AAVrh32.33, AAV-HSC15, AAV-HSC17, AAVhu.37, AAVrh.8, CHt-P6, AAV2.5, AAV6.2, AAV2i8, AAV-HSC15/17, AAVM41, AAV9.45, AAV6(Y445F/Y731F), AAV2.5T, AAV-HAE1/2, AAV clone 32/83, AAVShH10, AAV2 (Y->F), AAV8 (Y733F), AAV2.15, AAV2.4, AAVM41, and AAVr3.45. A non-limiting example of derivatives and pseudotypes that have chimeric VP1 proteins is rAAV2/5-1VP1u, which has the genome of AAV2, capsid backbone of AAV5 and VP1u of AAV1. Other non-limiting example of derivatives and pseudotypes that have chimeric VP1 proteins are rAAV2/5-8VP1u, rAAV2/9-1VP1u, and rAAV2/9-8VP1u.

AAV derivatives/pseudotypes, and methods of producing such derivatives/pseudotypes are known in the art (see, e.g., Mol Ther. 2012 April; 20(4):699-708. doi: 10.1038/mt.2011.287. Epub 2012 Jan. 24. The AAV vector toolkit: poised at the clinical crossroads. Asokan A1, Schaffer DV, Samulski RJ.). Methods for producing and using pseudotyped rAAV vectors are known in the art (see, e.g., Duan et al., J. Virol., 75:7662-7671, 2001; Halbert et al., J. Virol., 74:1524-1532, 2000; Zolotukhin et al., Methods, 28:158-167, 2002; and Auricchio et al., Hum. Molec. Genet., 10:3075-3081, 2001).

Methods of making or packaging rAAV particles are known in the art and reagents are commercially available (see, e.g., Zolotukhin et al. Production and purification of serotype 1, 2, and 5 recombinant adeno-associated viral vectors. Methods 28 (2002) 158-167; and U.S. Patent Publication Numbers US20070015238 and US20120322861, which are incorporated herein by reference; and plasmids and kits available from ATCC and Cell Biolabs, Inc.). For example, a plasmid comprising a gene of interest may be combined with one or more helper plasmids, e.g., that contain a rep gene (e.g., encoding Rep78, Rep68, Rep52 and Rep40) and a cap gene (encoding VP1, VP2, and VP3, including a modified VP2 region as described herein), and transfected into a recombinant cells such that the rAAV particle can be packaged and subsequently purified.

Recombinant AAV may comprise a nucleic acid vector, which may comprise at a minimum: (a) one or more heterologous nucleic acid regions comprising a sequence encoding a protein or polypeptide of interest or an RNA of interest (e.g., a siRNA or microRNA), and (b) one or more regions comprising inverted terminal repeat (ITR) sequences (e.g., wild-type ITR sequences or engineered ITR sequences) flanking the one or more nucleic acid regions (e.g., heterologous nucleic acid regions). Herein, heterologous nucleic acid regions comprising a sequence encoding a protein of interest or RNA of interest are referred to as genes of interest.

Any one of the rAAV particles provided herein may have capsid proteins that have amino acids of different serotypes outside of the VP1u region. In some embodiments, the serotype of the backbone of the VP1 protein is different from the serotype of the ITRs and/or the Rep gene. In some embodiments, the serotype of the backbone of the VP1 capsid protein of a particle is the same as the serotype of the ITRs. In some embodiments, the serotype of the backbone of the VP1 capsid protein of a particle is the same as the serotype of the Rep gene. In some embodiments, capsid proteins of rAAV particles comprise amino acid mutations that result in improved transduction efficiency.

In some embodiments, the nucleic acid vector comprises one or more regions comprising a sequence that facilitates expression of the nucleic acid (e.g., the heterologous nucleic acid), e.g., expression control sequences operatively linked to the nucleic acid. Numerous such sequences are known in the art. Non-limiting examples of expression control sequences include promoters, insulators, silencers, response elements, introns, enhancers, initiation sites, termination signals, and poly(A) tails. Any combination of such control sequences is contemplated herein (e.g., a promoter and an enhancer).

Final AAV constructs may incorporate a sequence encoding the gRNA. In other embodiments, the AAV constructs may incorporate a sequence encoding the second-site nicking guide RNA. In still other embodiments, the AAV constructs may incorporate a sequence encoding the second-site nicking guide RNA and a sequence encoding the gRNA.

In various embodiments, the gRNAs and the second-site nicking guide RNAs can be expressed from an appropriate promoter, such as a human U6 (hU6) promoter, a mouse U6 (mU6) promoter, or other appropriate promoter. The gRNAs and the second-site nicking guide RNAs can be driven by the same promoters or different promoters.

In some embodiments, a rAAV constructs or the herein compositions are administered to a subject enterally. In some embodiments, a rAAV constructs or the herein compositions are administered to the subject parenterally. In some embodiments, a rAAV particle or the herein compositions are administered to a subject subcutaneously, intraocularly, intravitreally, subretinally, intravenously (IV), intracerebro-ventricularly, intramuscularly, intrathecally (IT), intracisternally, intraperitoneally, via inhalation, topically, or by direct injection to one or more cells, tissues, or organs. In some embodiments, a rAAV particle or the herein compositions are administered to the subject by injection into the hepatic artery or portal vein.

In other aspects, the base editors can be divided at a split site and provided as two halves of a whole/complete base editor. The two halves can be delivered to cells (e.g., as expressed proteins or on separate expression vectors) and once in contact inside the cell, the two halves form the complete base editor through the self-splicing action of the inteins on each base editor half. Split intein sequences can be engineered into each of the halves of the encoded base editor to facilitate their transplicing inside the cell and the concomitant restoration of the complete, functioning base editor.

These split intein-based methods overcome several barriers to in vivo delivery. For example, the DNA encoding base editors is larger than the rAAV packaging limit, and so requires special solutions. One such solution is formulating the editor fused to split intein pairs that are packaged into two separate rAAV particles that, when co-delivered to a cell, reconstitute the functional editor protein. Several other special considerations to account for the unique features of base editing are described, including the optimization of second-site nicking targets and properly packaging base editors into virus vectors, including lentiviruses and rAAV.

In this aspect, the base editors can be divided at a split site and provided as two halves of a whole/complete base editor. The two halves can be delivered to cells (e.g., as expressed proteins or on separate expression vectors) and once in contact inside the cell, the two halves form the complete base editor through the self-splicing action of the inteins on each base editor half. Split intein sequences can be engineered into each of the halves of the encoded base editor to facilitate their transplicing inside the cell and the concomitant restoration of the complete, functioning base editor.

In various embodiments, the base editors may be engineered as two half proteins (i.e., a BE N-terminal half and a BE C-terminal half) by “splitting” the whole base editor as a “split site.” The “split site” refers to the location of insertion of split intein sequences (i.e., the N intein and the C intein) between two adjacent amino acid residues in the base editor. More specifically, the “split site” refers to the location of dividing the whole base editor into two separate halves, wherein in each halve is fused at the split site to either the N intein or the C intein motifs. The split site can be at any suitable location in the base editor fusion protein, but preferably the split site is located at a position that allows for the formation of two half proteins which are appropriately sized for delivery (e.g., by expression vector) and wherein the inteins, which are fused to each half protein at the split site termini, are available to sufficiently interact with one another when one half protein contacts the other half protein inside the cell.

In some embodiments, the split site is located in the napDNAbp domain. In other embodiments, the split site is located in the RT domain. In other embodiments, the split site is located in a linker that joins the napDNAbp domain and the RT domain.

In various embodiments, split site design requires finding sites to split and insert an N- and C-terminal intein that are both structurally permissive for purposes of packaging the two half base editor domains into two different AAV genomes. Additionally, intein residues necessary for trans splicing can be incorporated by mutating residues at the N terminus of the C terminal extein or inserting residues that will leave an intein “scar.”

In various embodiments, using SpCas9 nickase (SEQ ID NO: 29, 1368 amino acids) as an example, the split can between any two amino acids between 1 and 1368. Preferred splits, however, will be located between the central region of the protein, e.g., from amino acids 50-1250, or from 100-1200, or from 150-1150, or from 200-1100, or from 250-1050, or from 300-1000, or from 350-950, or from 400-900, or from 450-850, or from 500-800, or from 550-750, or from 600-700 of SEQ ID NO: 29. In specific exemplary embodiments, the split site may be between 740/741, or 801/802, or 1010/1011, or 1041/1042. In other embodiments the split site may be between 1/2, 2/3, 3/4, 4/5, 5/6, 6/7, 7/8, 8/9, 9/10, 10/11, 12/13, 14/15, 15/16, 17/18, 19/20 . . . 50/51 . . . 100/101 . . . 200/201 . . . 300/301 . . . 400/401 . . . 500/501 . . . 600/601 . . . 700/701 . . . 800/801 . . . 900/901 . . . 1000/1001 . . . 1100/1101 . . . 1200/1201 . . . 1300/1301 . . . and 1367/1368, including all adjacent pairs of amino acid residues.

In various embodiments, the split intein sequences can be engineered by from the following intein sequences.

2-4 INTEIN:
(SEQ ID NO: 164)
CLAEGTRIFDPVTGTTHRIEDVVDGRKPIHVVAAA
KDGTLLARPVVSWFDQGTRDVIGLRIAGGAIVWAT
PDHKVLTEYGWRAAGELRKGDRVAGPGGSGNSLAL
SLTADQMVSALLDAEPPILYSEYDPTSPFSEASMM
GLLTNLADRELVHMINWAKRVPGFVDLTLHDQAHL
LECAWLEILMIGLVWRSMEHPGKLLFAPNLLLDRN
QGKCVEGMVEIFDMLLATSSRFRMMNLQGEEFVCL
KSIILLNSGVYTFLSSTLKSLEEKDHIHRALDKIT
DTLIHLMAKAGLTLQQQHQRLAQLLLILSHIRHMS
NKGMEHLYSMKYKNVVPLYDLLLEMLDAHRLHAGG
SGASRVQAFADALDDKFLHDMLAEELRYSVIREVL
PTRRARTFDLEVEELHTLVAEGVVVHNC
3-2 INTEIN
(SEQ ID NO: 165)
CLAEGTRIFDPVTGTTHRIEDVVDGRKPIHVVAVA
KDGTLLARPVVSWFDQGTRDVIGLRIAGGAIVWAT
PDHKVLTEYGWRAAGELRKGDRVAGPGGSGNSLAL
SLTADQMVSALLDAEPPILYSEYDPTSPFSEASMM
GLLTNLADRELVHMINWAKRVPGFVDLTLHDQAHL
LECAWLEILMIGLVWRSMEHPGKLLFAPNLLLDRN
QGKCVEGMVEIFDMLLATSSRFRMMNLQGEEFVCL
KSIILLNSGVYTFLSSTLKSLEEKDHIHRALDKIT
DTLIHLMAKAGLTLQQQHQRLAQLLLILSHIRHMS
NKGMEHLYSMKYTNVVPLYDLLLEMLDAHRLHAGG
SGASRVQAFADALDDKFLHDMLAEELRYSVIREVL
PTRRARTFDLEVEELHTLVAEGVVVHNC
30R3-1 INTEIN
(SEQ ID NO: 166)
CLAEGTRIFDPVTGTTHRIEDVVDGRKPIHVVAAA
KDGTLLARPVVSWFDQGTRDVIGLRIAGGATVWAT
PDHKVLTEYGWRAAGELRKGDRVAGPGGSGNSLAL
SLTADQMVSALLDAEPPIPYSEYDPTSPFSEASMM
GLLTNLADRELVHMINWAKRVPGFVDLTLHDQAHL
LECAWLEILMIGLVWRSMEHPGKLLFAPNLLLDRN
QGKCVEGMVEIFDMLLATSSRFRMMNLQGEEFVCL
KSIILLNSGVYTFLSSTLKSLEEKDHIHRALDKIT
DTLIHLMAKAGLTLQQQHQRLAQLLLILSHIRHMS
NKGMEHLYSMKYKNVVPLYDLLLEMLDAHRLHAGG
SGASRVQAFADALDDKFLHDMLAEGLRYSVIREVL
PTRRARTFDLEVEELHTLVAEGVVVHNC
30R3-2 INTEIN
(SEQ ID NO: 167)
CLAEGTRIFDPVTGTTHRIEDVVDGRKPIHVVAAA
KDGTLLARPVVSWFDQGTRDVIGLRIAGGATVWAT
PDHKVLTEYGWRAAGELRKGDRVAGPGGSGNSLAL
SLTADQMVSALLDAEPPILYSEYDPTSPFSEASMM
GLLTNLADRELVHMINWAKRVPGFVDLTLHDQAHL
LECAWLEILMIGLVWRSMEHPGKLLFAPNLLLDRN
QGKCVEGMVEIFDMLLATSSRFRMMNLQGEEFVCL
KSIILLNSGVYTFLSSTLKSLEEKDHIHRALDKIT
DTLIHLMAKAGLTLQQQHQRLAQLLLILSHIRHMS
NKGMEHLYSMKYKNVVPLYDLLLEMLDAHRLHAGG
SGASRVQAFADALDDKFLHDMLAEELRYSVIREVL
PTRRARTFDLEVEELHTLVAEGVVVHNC
30R3-3 INTEIN
(SEQ ID NO: 168)
CLAEGTRIFDPVTGTTHRIEDVVDGRKPIHVVAAA
KDGTLLARPVVSWFDQGTRDVIGLRIAGGATVWAT
PDHKVLTEYGWRAAGELRKGDRVAGPGGSGNSLAL
SLTADQMVSALLDAEPPIPYSEYDPTSPFSEASMM
GLLTNLADRELVHMINWAKRVPGFVDLTLHDQAHL
LECAWLEILMIGLVWRSMEHPGKLLFAPNLLLDRN
QGKCVEGMVEIFDMLLATSSRFRMMNLQGEEFVCL
KSIILLNSGVYTFLSSTLKSLEEKDHIHRALDKIT
DTLIHLMAKAGLTLQQQHQRLAQLLLILSHIRHMS
NKGMEHLYSMKYKNVVPLYDLLLEMLDAHRLHAGG
SGASRVQAFADALDDKFLHDMLAEELRYSVIREVL
PTRRARTFDLEVEELHTLVAEGVVVHNC
37R3-1 INTEIN
((SEQ ID NO: 169)
CLAEGTRIFDPVTGTTHRIEDVVDGRKPIHVVAAA
KDGTLLARPVVSWFDQGTRDVIGLRIAGGATVWAT
PDHKVLTEYGWRAAGELRKGDRVAGPGGSGNSLAL
SLTADQMVSALLDAEPPILYSEYNPTSPFSEASMM
GLLTNLADRELVHMINWAKRVPGFVDLTLHDQAHL
LERAWLEILMIGLVWRSMEHPGKLLFAPNLLLDRN
QGKCVEGMVEIFDMLLATSSRFRMMNLQGEEFVCL
KSIILLNSGVYTFLSSTLKSLEEKDHIHRALDKIT
DTLIHLMAKAGLTLQQQHQRLAQLLLILSHIRHMS
NKGMEHLYSMKYKNVVPLYDLLLEMLDAHRLHAGG
SGASRVQAFADALDDKFLHDMLAEGLRYSVIREVL
PTRRARTFDLEVEELHTLVAEGVVVHNC
37R3-2 INTEIN
(SEQ ID NO: 170)
CLAEGTRIFDPVTGTTHRIEDVVDGRKPIHVVAAA
KDGTLLARPVVSWFDQGTRDVIGLRIAGGAIVWAT
PDHKVLTEYGWRAAGELRKGDRVAGPGGSGNSLAL
SLTADQMVSALLDAEPPILYSEYDPTSPFSEASMM
GLLTNLADRELVHMINWAKRVPGFVDLTLHDQAHL
LERAWLEILMIGLVWRSMEHPGKLLFAPNLLLDRN
QGKCVEGMVEIFDMLLATSSRFRMMNLQGEEFVCL
KSIILLNSGVYTFLSSTLKSLEEKDHIHRALDKIT
DTLIHLMAKAGLTLQQQHQRLAQLLLILSHIRHMS
NKGMEHLYSMKYKNVVPLYDLLLEMLDAHRLHAGG
SGASRVQAFADALDDKFLHDMLAEGLRYSVIREVL
PTRRARTFDLEVEELHTLVAEGVVVHNC
37R3-3 INTEIN
(SEQ ID NO: 171)
CLAEGTRIFDPVTGTTHRIEDVVDGRKPIHVVAVA
KDGTLLARPVVSWFDQGTRDVIGLRIAGGATVWAT
PDHKVLTEYGWRAAGELRKGDRVAGPGGSGNSLAL
SLTADQMVSALLDAEPPILYSEYDPTSPFSEASMM
GLLTNLADRELVHMINWAKRVPGFVDLTLHDQAHL
LERAWLEILMIGLVWRSMEHPGKLLFAPNLLLDRN
QGKCVEGMVEIFDMLLATSSRFRMMNLQGEEFVCL
KSIILLNSGVYTFLSSTLKSLEEKDHIHRALDKIT
DTLIHLMAKAGLTLQQQHQRLAQLLLILSHIRHMS
NKGMEHLYSMKYKNVVPLYDLLLEMLDAHRLHAGG
SGASRVQAFADALDDKFLHDMLAEELRYSVIREVL
PTRRARTFDLEVEELHTLVAEGVVVHNC

In various embodiments, the split inteins can be used to separately deliver separate portions of a complete Base editor fusion protein to a cell, which upon expression in a cell, become reconstituted as a complete Base editor fusion protein through the trans splicing.

In some embodiments, the disclosure provides a method of delivering a Base editor fusion protein to a cell, comprising: constructing a first expression vector encoding an N-terminal fragment of the Base editor fusion protein fused to a first split intein sequence; constructing a second expression vector encoding a C-terminal fragment of the Base editor fusion protein fused to a second split intein sequence; delivering the first and second expression vectors to a cell, wherein the N-terminal and C-terminal fragment are reconstituted as the Base editor fusion protein in the cell as a result of trans splicing activity causing self-excision of the first and second split intein sequences.

In other embodiments, the split site is in the napDNAbp domain.

In still other embodiments, the split site is in the adenosine deaminase domain.

In yet other embodiments, the split site is in the linker.

In other embodiments, the base editors may be delivered by ribonucleoprotein complexes.

In this aspect, the base editors may be delivered by non-viral delivery strategies involving delivery of a base editor complexed with a gRNA (i.e., a BE ribonucleoprotein complex) by various methods, including electroporation and lipid nanoparticles. Methods of non-viral delivery of nucleic acids include lipofection, nucleofection, microinjection, biolistics, virosomes, liposomes, immunoliposomes, polycation or lipid:nucleic acid conjugates, naked DNA, artificial virions, and agent-enhanced uptake of DNA. Lipofection is described in e.g., U.S. Pat. Nos. 5,049,386, 4,946,787; and 4,897,355) and lipofection reagents are sold commercially (e.g., Transfectam™ and Lipofectin™). Cationic and neutral lipids that are suitable for efficient receptor-recognition lipofection of polynucleotides include those of Feigner, WO 91/17424; WO 91/16024. Delivery can be to cells (e.g. in vitro or ex vivo administration) or target tissues (e.g. in vivo administration).

The preparation of lipid:nucleic acid complexes, including targeted liposomes such as immunolipid complexes, is well known to one of skill in the art (see, e.g., Crystal, Science 270:404-410 (1995); Blaese et al., Cancer Gene Ther. 2:291-297 (1995); Behr et al., Bioconjugate Chem. 5:382-389 (1994); Remy et al., Bioconjugate Chem. 5:647-654 (1994); Gao et al., Gene Therapy 2:710-722 (1995); Ahmad et al., Cancer Res. 52:4817-4820 (1992); U.S. Pat. Nos. 4,186,183, 4,217,344, 4,235,871, 4,261,975, 4,485,054, 4,501,728, 4,774,085, 4,837,028, and 4,946,787).

XI. Pharmaceutical Compositions

Other aspects of the present disclosure relate to pharmaceutical compositions comprising any of the adenosine deaminases, fusion proteins, or the fusion protein-gRNA complexes described herein. The term “pharmaceutical composition”, as used herein, refers to a composition formulated for pharmaceutical use. In some embodiments, the pharmaceutical composition further comprises a pharmaceutically acceptable carrier. In some embodiments, the pharmaceutical composition comprises additional agents (e.g. for specific delivery, increasing half-life, or other therapeutic compounds).

As used here, the term “pharmaceutically-acceptable carrier” means a pharmaceutically-acceptable material, composition or vehicle, such as a liquid or solid filler, diluent, excipient, manufacturing aid (e.g., lubricant, talc magnesium, calcium or zinc stearate, or steric acid), or solvent encapsulating material, involved in carrying or transporting the compound from one site (e.g., the delivery site) of the body, to another site (e.g., organ, tissue or portion of the body). A pharmaceutically acceptable carrier is “acceptable” in the sense of being compatible with the other ingredients of the formulation and not injurious to the tissue of the subject (e.g., physiologically compatible, sterile, physiologic pH, etc.). Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, methylcellulose, ethyl cellulose, microcrystalline cellulose and cellulose acetate; (4) powdered tragacanth; (5) malt; (6) gelatin; (7) lubricating agents, such as magnesium stearate, sodium lauryl sulfate and talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol (PEG); (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates and/or polyanhydrides; (22) bulking agents, such as polypeptides and amino acids (23) serum component, such as serum albumin, HDL and LDL; (22) C2-C12 alcohols, such as ethanol; and (23) other non-toxic compatible substances employed in pharmaceutical formulations. Wetting agents, coloring agents, release agents, coating agents, sweetening agents, flavoring agents, perfuming agents, preservative and antioxidants can also be present in the formulation. The terms such as “excipient”, “carrier”, “pharmaceutically acceptable carrier” or the like are used interchangeably herein.

In some embodiments, the pharmaceutical composition is formulated for delivery to a subject, e.g., for gene editing. Suitable routes of administrating the pharmaceutical composition described herein include, without limitation: topical, subcutaneous, transdermal, intradermal, intralesional, intraarticular, intraperitoneal, intravesical, transmucosal, gingival, intradental, intracochlear, transtympanic, intraorgan, epidural, intrathecal, intramuscular, intravenous, intravascular, intraosseus, periocular, intratumoral, intracerebral, and intracerebroventricular administration.

In some embodiments, the pharmaceutical composition described herein is administered locally to a diseased site (e.g., tumor site). In some embodiments, the pharmaceutical composition described herein is administered to a subject by injection, by means of a catheter, by means of a suppository, or by means of an implant, the implant being of a porous, non-porous, or gelatinous material, including a membrane, such as a sialastic membrane, or a fiber.

In other embodiments, the pharmaceutical composition described herein is delivered in a controlled release system. In one embodiment, a pump may be used (see, e.g., Langer, 1990, Science 249:1527-1533; Sefton, 1989, CRC Crit. Ref. Biomed. Eng. 14:201; Buchwald et al., 1980, Surgery 88:507; Saudek et al., 1989, N. Engl. J. Med. 321:574). In another embodiment, polymeric materials can be used. (See, e.g., Medical Applications of Controlled Release (Langer and Wise eds., CRC Press, Boca Raton, Fla., 1974); Controlled Drug Bioavailability, Drug Product Design and Performance (Smolen and Ball eds., Wiley, New York, 1984); Ranger and Peppas, 1983, Macromol. Sci. Rev. Macromol. Chem. 23:61. See also Levy et al., 1985, Science 228:190; During et al., 1989, Ann. Neurol. 25:351; Howard et al., 1989, J. Neurosurg. 71:105.). Other controlled release systems are discussed, for example, in Langer, supra.

In some embodiments, the pharmaceutical composition is formulated in accordance with routine procedures as a composition adapted for intravenous or subcutaneous administration to a subject, e.g., a human. In some embodiments, pharmaceutical compositions for administration by injection are solutions in sterile isotonic aqueous buffer. Where necessary, the pharmaceutical can also include a solubilizing agent and a local anesthetic such as lignocaine to ease pain at the site of the injection. Generally, the ingredients are supplied either separately or mixed together in unit dosage form, for example, as a dry lyophilized powder or water free concentrate in a hermetically sealed container such as an ampoule or sachette indicating the quantity of active agent. Where the pharmaceutical is to be administered by infusion, it can be dispensed with an infusion bottle containing sterile pharmaceutical grade water or saline. Where the pharmaceutical composition is administered by injection, an ampoule of sterile water for injection or saline can be provided so that the ingredients can be mixed prior to administration.

A pharmaceutical composition for systemic administration may be a liquid, e.g., sterile saline, lactated Ringer's or Hank's solution. In addition, the pharmaceutical composition can be in solid forms and re-dissolved or suspended immediately prior to use. Lyophilized forms are also contemplated.

A pharmaceutical composition for systemic administration may be a liquid, e.g., sterile saline, lactated Ringer's or Hank's solution. In addition, the pharmaceutical composition can be in solid forms and re-dissolved or suspended immediately prior to use. Lyophilized forms are also contemplated.

The pharmaceutical composition can be contained within a lipid particle or vesicle, such as a liposome or microcrystal, which is also suitable for parenteral administration. The particles can be of any suitable structure, such as unilamellar or plurilamellar, so long as compositions are contained therein. Compounds can be entrapped in “stabilized plasmid-lipid particles” (SPLP) containing the fusogenic lipid dioleoylphosphatidylethanolamine (DOPE), low levels (5-10 mol %) of cationic lipid, and stabilized by a polyethyleneglycol (PEG) coating (Zhang Y. P. et al., Gene Ther. 1999, 6:1438-47). Positively charged lipids such as N-[1-(2,3-dioleoyloxi)propyl]-N,N,N-trimethyl-amoniummethylsulfate, or “DOTAP,” are particularly preferred for such particles and vesicles. The preparation of such lipid particles is well known. See, e.g., U.S. Pat. Nos. 4,880,635; 4,906,477; 4,911,928; 4,917,951; 4,920,016; and 4,921,757; each of which is incorporated herein by reference.

The pharmaceutical composition described herein may be administered or packaged as a unit dose, for example. The term “unit dose” when used in reference to a pharmaceutical composition of the present disclosure refers to physically discrete units suitable as unitary dosage for the subject, each unit containing a predetermined quantity of active material calculated to produce the desired therapeutic effect in association with the required diluent; i.e., carrier, or vehicle.

Further, the pharmaceutical composition can be provided as a pharmaceutical kit comprising (a) a container containing a compound of the invention in lyophilized form and (b) a second container containing a pharmaceutically acceptable diluent (e.g., sterile water) for injection. The pharmaceutically acceptable diluent can be used for reconstitution or dilution of the lyophilized compound of the invention. Optionally associated with such container(s) can be a notice in the form prescribed by a governmental agency regulating the manufacture, use or sale of pharmaceuticals or biological products, which notice reflects approval by the agency of manufacture, use or sale for human administration.

In another aspect, an article of manufacture containing materials useful for the treatment of the diseases described above is included. In some embodiments, the article of manufacture comprises a container and a label. Suitable containers include, for example, bottles, vials, syringes, and test tubes. The containers may be formed from a variety of materials such as glass or plastic. In some embodiments, the container holds a composition that is effective for treating a disease described herein and may have a sterile access port. For example, the container may be an intravenous solution bag or a vial having a stopper pierceable by a hypodermic injection needle. The active agent in the composition is a compound of the invention. In some embodiments, the label on or associated with the container indicates that the composition is used for treating the disease of choice. The article of manufacture may further comprise a second container comprising a pharmaceutically-acceptable buffer, such as phosphate-buffered saline, Ringer's solution, or dextrose solution. It may further include other materials desirable from a commercial and user standpoint, including other buffers, diluents, filters, needles, syringes, and package inserts with instructions for use.

XII. Kits, Vectors, Cells

Some aspects of this disclosure provide kits comprising a nucleic acid construct comprising a nucleotide sequence encoding a base editor, or a component thereof, including a cytidine deaminase, adenosine deaminase, or an napDNAbp, and/or a guide RNA, for editing a target DNA in a cell. In some embodiments, the nucleotide sequence encodes any of the napDNAbps, cytidine deaminases, and/or adenosine deaminases, and/or guide RNAs provided herein. In some embodiments, the nucleotide sequence comprises a heterologous promoter that drives expression of the napDNAbps, cytidine deaminases, and/or adenosine deaminases, and/or guide RNAs described herein. The nucleotide sequence may further comprise one or more heterologous promoters that drive expression of the napDNAbps, cytidine deaminases, and/or adenosine deaminases, and/or guide RNAs, either from the same nucleotide sequence or separate nucleotide sequences.

In some embodiments, the kit further comprises an expression construct encoding a guide nucleic acid backbone, e.g., a guide RNA backbone, wherein the construct comprises a cloning site positioned to allow the cloning of a nucleic acid sequence identical or complementary to a target sequence into the guide nucleic acid, e.g., guide RNA backbone.

The disclosure further provides kits comprising a nucleic acid construct, comprising (a) a nucleotide sequence encoding a napDNAbp (e.g., a Cas9 domain) fused to a deaminase, or a base editor comprising a napDNAbp (e.g., Cas9 domain) and a deaminase as provided herein; and (b) a heterologous promoter that drives expression of the sequence of (a). In some embodiments, the kit further comprises an expression construct encoding a guide nucleic acid backbone, (e.g., a guide RNA backbone), wherein the construct comprises a cloning site positioned to allow the cloning of a nucleic acid sequence identical or complementary to a target sequence into the guide nucleic acid (e.g., guide RNA backbone).

Some embodiments of this disclosure provide cells comprising any of the base editors or complexes provided herein. In some embodiments, the cells comprise nucleotide constructs that encode any of the base editors provided herein. In some embodiments, the cells comprise any of the nucleotides or vectors provided herein. In some embodiments, a host cell is transiently or non-transiently transfected with one or more vectors described herein. In some embodiments, a cell is transfected as it naturally occurs in a subject. In some embodiments, a cell that is transfected is taken from a subject. In some embodiments, the cell is derived from cells taken from a subject, such as a cell line. A wide variety of cell lines for tissue culture are known in the art.

In some embodiments, a host cell is transiently or non-transiently transfected with one or more vectors described herein. In some embodiments, a cell is transfected as it naturally occurs in a subject. In some embodiments, a cell that is transfected is taken from a subject. In some embodiments, the cell is derived from cells taken from a subject, such as a cell line. A wide variety of cell lines for tissue culture are known in the art. Examples of cell lines include, but are not limited to, C8161, CCRF-CEM, MOLT, mIMCD-3, NHDF, HeLa-S3, Huh1, Huh4, Huh7, HUVEC, HASMC, HEKn, HEKa, MiaPaCell, Panc1, PC-3, TF1, CTLL-2, C1R, Rat6, CV1, RPTE, A10, T24, J82, A375, ARH-77, Calu1, SW480, SW620, SKOV3, SK-UT, CaCo2, P388D1, SEM-K2, WEHI-231, HB56, TIB55, Jurkat, J45.01, LRMB, Bcl-1, BC-3, IC21, DLD2, Raw264.7, NRK, NRK-52E, MRC5, MEF, Hep G2, HeLa B, HeLa T4, COS, COS-1, COS-6, COS-M6A, BS-C-1 monkey kidney epithelial, BALB/3T3 mouse embryo fibroblast, 3T3 Swiss, 3T3-L1, 132-d5 human fetal fibroblasts; 10.1 mouse fibroblasts, 293-T, 3T3, 721, 9L, A2780, A2780ADR, A2780cis, A 172, A20, A253, A431, A-549, ALC, B16, B35, BCP-1 cells, BEAS-2B, bEnd.3, BHK-21, BR 293. BxPC3. C3H-10T1/2, C6/36, Cal-27, CHO, CHO-7, CHO-IR, CHO-K1, CHO-K2, CHO-T, CHO Dhfr −/−, COR-L23, COR-L23/CPR, COR-L23/5010, COR-L23/R23, COS-7, COV-434, CML T1, CMT, CT26, D17, DH82, DU145, DuCaP, EL4, EM2, EM3, EMT6/AR1, EMT6/AR10.0, FM3, H1299, H69, HB54, HB55, HCA2, HEK-293, HeLa, Hepa1c1c7, HL-60, HMEC, HT-29, Jurkat, JY cells, K562 cells, Ku812, KCL22, KG1, KYO1, LNCap, Ma-Mel 1-48, MC-38, MCF-7, MCF-10A, MDA-MB-231, MDA-MB-468, MDA-MB-435, MDCK II, MDCK 11, MOR/0.2R, MONO-MAC 6, MTD-1A, MyEnd, NCI-H69/CPR, NCI-H69/LX10, NCI-H69/LX20, NCI-H69/LX4, NIH-3T3, NALM-1, NW-145, OPCN/OPCT cell lines, Peer, PNT-1A/PNT 2, RenCa, RIN-5F, RMA/RMAS, Saos-2 cells, Sf-9, SkBr3, T2, T-47D, T84, THP1 cell line, U373, U87, U937, VCaP, Vero cells, WM39, WT-49, X63, YAC-1, YAR, and transgenic varieties thereof. Cell lines are available from a variety of sources known to those with skill in the art (see, e.g., the American Type Culture Collection (ATCC) (Manassas, Va.)). In some embodiments, a cell transfected with one or more vectors described herein is used to establish a new cell line comprising one or more vector-derived sequences. In some embodiments, a cell transiently transfected with the components of a CRISPR system as described herein (such as by transient transfection of one or more vectors, or transfection with RNA), and modified through the activity of a CRISPR complex, is used to establish a new cell line comprising cells containing the modification but lacking any other exogenous sequence. In some embodiments, cells transiently or non-transiently transfected with one or more vectors described herein, or cell lines derived from such cells are used in assessing one or more test compounds.

In some aspects, the present disclosure provides uses of any one of the base editors described herein and a guide RNA targeting this base editor to a target A:T base pair in a nucleic acid molecule in the manufacture of a kit for nucleic acid editing, wherein the nucleic acid editing comprises contacting the nucleic acid molecule with the base editor and guide RNA under conditions suitable for the substitution of the adenine (A) of the A:T nucleobase pair with an guanine (G). In some embodiments of these uses, the nucleic acid molecule is a double-stranded DNA molecule. In some embodiments, the step of contacting of induces separation of the double-stranded DNA at a target region. In some embodiments, the step of contacting further comprises nicking one strand of the double-stranded DNA, wherein the one strand comprises an unmutated strand that comprises the T of the target A:T nucleobase pair.

In some aspects, the present disclosure provides uses of any one of the base editors described herein and a guide RNA targeting this base editor to a target A:T base pair in a nucleic acid molecule in the manufacture of a kit for evaluating the off-target effects of a base editor, wherein the step of evaluating the off-target effects comprises contacting the base editor with the nucleic acid molecule and determining off-target effects in accordance with any one of the disclosed methods. In some embodiments of these uses, the nucleic acid molecule is a double-stranded DNA molecule. In some embodiments, the step of contacting of induces separation of the double-stranded DNA at a target region. In some embodiments, the step of contacting further comprises nicking one strand of the double-stranded DNA, wherein the one strand comprises an unmutated strand that comprises the T of the target A:T nucleobase pair.

In some embodiments of the described uses, the step of contacting is performed in vitro. In other embodiments, the step of contacting is performed in vivo. In some embodiments, the step of contacting is performed in a subject (e.g., a human subject or a non-human animal subject). In some embodiments, the step of contacting is performed in a cell, such as a human or non-human animal cell.

The present disclosure also provides uses of any one of the base editors described herein as a medicament. The present disclosure also provides uses of any one of the complexes of base editors and guide RNAs described herein as a medicament.

Some aspects of this disclosure provide kits comprising a nucleic acid construct comprising a nucleotide sequence encoding an adenosine deaminase capable of deaminating an adenosine in a deoxyribonucleic acid (DNA) molecule. In some embodiments, the nucleotide sequence encodes any of the adenosine deaminases provided herein. In some embodiments, the nucleotide sequence comprises a heterologous promoter that drives expression of the adenosine deaminase.

Some aspects of this disclosure provide kits comprising a nucleic acid construct, comprising (a) a nucleotide sequence encoding a napDNAbp (e.g., a Cas9 domain) fused to an adenosine deaminase, or a fusion protein comprising a napDNAbp (e.g., Cas9 domain) and an adenosine deaminase as provided herein; and (b) a heterologous promoter that drives expression of the sequence of (a). In some embodiments, the kit further comprises an expression construct encoding a guide nucleic acid backbone, (e.g., a guide RNA backbone), wherein the construct comprises a cloning site positioned to allow the cloning of a nucleic acid sequence identical or complementary to a target sequence into the guide nucleic acid (e.g., guide RNA backbone).

Some aspects of this disclosure provide cells comprising any of the adenosine deaminases, fusion proteins, or complexes provided herein. In some embodiments, the cells comprise a nucleotide that encodes any of the adenosine deaminases or fusion proteins provided herein. In some embodiments, the cells comprise any of the nucleotides or vectors provided herein.

The description of exemplary embodiments of the systems described above is provided for illustration purposes only and not meant to be limiting. Additional systems, e.g., variations of the exemplary systems described in detail above, are also embraced by this disclosure.

It should be appreciated however, that additional fusion proteins would be apparent to the skilled artisan based on the present disclosure and knowledge in the art.

The function and advantage of these and other embodiments of the present invention will be more fully understood from the Examples below. The following Examples are intended to illustrate the benefits of the present invention and to describe particular embodiments, but are not intended to exemplify the full scope of the invention. Accordingly, it will be understood that the Examples are not meant to limit the scope of the invention.

### SEQUENCES

The following sequences appear in and form a part of this disclosure.

napDNAbp

| | | |
|---|---|---|
| | | SEQ ID |
| DESCRIPTION | SEQUENCE | NO: |

SpCas9 wild type

| | | |
|---|---|---|
| SpCas9 | MDKKYSIGLDIGTNSVGWAVITDEYKVPSKKFKVLGNTDRHSIKKNLIGALLFDSGETAEATRLKRTA | 5 |
| Streptococcus | RRRYTRRKNRICYLQEIFSNEMAKVDDSFFHRLEESFLVEEDKKHERHPIFGNIVDEVAYHEKYPTIY | |
| pyogenes Ml | HLRKKLVDSTDKADLRLIYLALAHMIKFRGHFLIEGDLNPDNSDVDKLFIQLVQTYNQLFEENPINAS | |
| SwissProt | GVDAKAILSARLSKSRRLENLIAQLPGEKKNGLFGNLIALSLGLTPNFKSNFDLAEDAKLQLSKDTYD | |
| Accession No. | DDLDNLLAQIGDQYADLFLAAKNLSDAILLSDILRVNTEITKAPLSASMIKRYDEHHQDLTLLKALVR | |
| Q99ZW2 | QQLPEKYKEIFFDQSKNGYAGYIDGGASQEEFYKFIKPILEKMDGTEELLVKLNREDLLRKQRTFDNG | |
| Wild type | SIPHQIHLGELHAILRRQEDFYPFLKDNREKIEKILTFRIPYYVGPLARGNSRFAWMTRKSEETITPW | |
| | NFEEVVDKGASAQSFIERMTNFDKNLPNEKVLPKHSLLYEYFTVYNELTKVKYVTEGMRKPAFLSGEQ | |
| | KKAIVDLLFKTNRKVTVKQLKEDYFKKIECFDSVEISGVEDRFNASLGTYHDLLKIIKDKDFLDNEEN | |
| | EDILEDIVLTLTLFEDREMIEERLKTYAHLFDDKVMKQLKRRRYTGWGRLSRKLINGIRDKQSGKTIL | |
| | DFLKSDGFANRNFMQLIHDDSLTFKEDIQKAQVSGQGDSLHEHIANLAGSPAIKKGILQTVKVVDELV | |
| | KVMGRHKPENIVIEMARENQTTQKGQKNSRERMKRIEEGIKELGSQILKEHPVENTQLQNEKLYLYYL | |
| | QNGRDMYVDQELDINRLSDYDVDHIVPQSFLKDDSIDNKVLTRSDKNRGKSDNVPSEEVVKKMKNYWR | |
| | QLLNAKLITQRKFDNLTKAERGGLSELDKAGFIKRQLVETRQITKHVAQILDSRMNTKYDENDKLIRE | |
| | VKVITLKSKLVSDFRKDFQFYKVREINNYHHAHDAYLNAVVGTALIKKYPKLESEFVYGDYKVYDVRK | |
| | MIAKSEQEIGKATAKYFFYSNIMNFFKTEITLANGEIRKRPLIETNGETGEIVWDKGRDFATVRKVLS | |
| | MPQVNIVKKTEVQTGGFSKESILPKRNSDKLIARKKDWDPKKYGGFDSPTVAYSVLVVAKVEKGKSKK | |
| | LKSVKELLGITIMERSSFEKNPIDFLEAKGYKEVKKDLIIKLPKYSLFELENGRKRMLASAGELQKGN | |
| | ELALPSKYVNFLYLASHYEKLKGSPEDNEQKQLFVEQHKHYLDEIIEQISEFSKRVILADANLDKVLS | |
| | AYNKHRDKPIREQAENIIHLFTLTNLGAPAAFKYFDTTIDRKRYTSTKEVLDATLIHQSITGLYETRI | |
| | DLSQLGGD | |
| SpCas9 | ATGGATAAAAAATATAGCATTGGCCTGGATATTGGCACCAACAGCGTGGGCTGGGCGGTGATTACCGA | 6 |
| Reverse | TGAATATAAAGTGCCGAGCAAAAAATTTAAAGTGCTGGGCAACACCGATCGCCATAGCATTAAAAAAA | |
| translation of | ACCTGATTGGCGCGCTGCTGTTTGATAGCGGCGAAACCGCGGAAGCGACCCGCCTGAAACGCACCGCG | |
| SwissProt | CGCCGCCGCTATACCCGCCGCAAAAACCGCATTTGCTATCTGCAGGAAATTTTTAGCAACGAAATGGC | |
| Accession No. | GAAAGTGGATGATAGCTTTTTTCATCGCCTGGAAGAAAGCTTTCTGGTGGAAGAAGATAAAAAACATG | |
| Q99ZW2 | AACGCCATCCGATTTTTGGCAACATTGTGGATGAAGTGGCGTATCATGAAAAATATCCGACCATTTAT | |
| Streptococcus | CATCTGCGCAAAAAACTGGTGGATAGCACCGATAAAGCGGATCTGCGCCTGATTTATCTGGCGCTGGC | |
| pyogenes | GCATATGATTAAATTTCGCGGCCATTTTCTGATTGAAGGCGATCTGAACCCGGATAACAGCGATGTGG | |
| | ATAAACTGTTTATTCAGCTGGTGCAGACCTATAACCAGCTGTTTGAAGAAAACCCGATTAACGCGAGC | |
| | GGCGTGGATGCGAAAGCGATTCTGAGCGCGCGCCTGAGCAAAAGCCGCCGCCTGGAAAACCTGATTGC | |
| | GCAGCTGCCGGGCGAAAAAAAAAACGGCCTGTTTGGCAACCTGATTGCGCTGAGCCTGGGCCTGACCC | |
| | CGAACTTTAAAAGCAACTTTGATCTGGCGGAAGATGCGAAACTGCAGCTGAGCAAAGATACCTATGAT | |
| | GATGATCTGGATAACCTGCTGGCGCAGATTGGCGATCAGTATGCGGATCTGTTTCTGGCGGCGAAAAA | |
| | CCTGAGCGATGCGATTCTGCTGAGCGATATTCTGCGCGTGAACACCGAAATTACCAAAGCGCCGCTGA | |
| | GCGCGAGCATGATTAAACGCTATGATGAACATCATCAGGATCTGACCCTGCTGAAAGCGCTGGTGCGC | |
| | CAGCAGCTGCCGGAAAAATATAAAGAAATTTTTTTTGATCAGAGCAAAAACGGCTATGCGGGCTATAT | |
| | TGATGGCGGCGCGAGCCAGGAAGAATTTTATAAATTTATTAAACCGATTCTGGAAAAAATGGATGGCA | |
| | CCGAAGAACTGCTGGTGAAACTGAACCGCGAAGATCTGCTGCGCAAACAGCGCACCTTTGATAACGGC | |
| | AGCATTCCGCATCAGATTCATCTGGGCGAACTGCATGCGATTCTGCGCCGCCAGGAAGATTTTTATCC | |
| | GTTTCTGAAAGATAACCGCGAAAAAATTGAAAAAATTCTGACCTTTCGCATTCCGTATTATGTGGGCC | |
| | CGCTGGCGCGCGGCAACAGCCGCTTTGCGTGGATGACCCGCAAAAGCGAAGAAACCATTACCCCGTGG | |
| | AACTTTGAAGAAGTGGTGGATAAAGGCGCGAGCGCGCAGAGCTTTATTGAACGCATGACCAACTTTGA | |
| | TAAAAACCTGCCGAACGAAAAAGTGCTGCCGAAACATAGCCTGCTGTATGAATATTTTACCGTGTATA | |
| | ACGAACTGACCAAAGTGAAATATGTGACCGAAGGCATGCGCAAACCGGCGTTTCTGAGCGGCGAACAG | |
| | AAAAAAGCGATTGTGGATCTGCTGTTTAAAACCAACCGCAAAGTGACCGTGAAACAGCTGAAAGAAGA | |
| | TTATTTTAAAAAAATTGAATGCTTTGATAGCGTGGAAATTAGCGGCGTGGAAGATCGCTTTAACGCGA | |
| | GCCTGGGCACCTATCATGATCTGCTGAAAATTATTAAAGATAAAGATTTTCTGGATAACGAAGAAAAC | |
| | GAAGATATTCTGGAAGATATTGTGCTGACCCTGACCCTGTTTGAAGATCGCGAAATGATTGAAGAACG | |
| | CCTGAAAACCTATGCGCATCTGTTTGATGATAAAGTGATGAAACAGCTGAAACGCCGCCGCTATACCG | |
| | GCTGGGGCCGCCTGAGCCGCAAACTGATTAACGGCATTCGCGATAAACAGAGCGGCAAAACCATTCTG | |
| | GATTTTCTGAAAAGCGATGGCTTTGCGAACCGCAACTTTATGCAGCTGATTCATGATGATAGCCTGAC | |
| | CTTTAAAGAAGATATTCAGAAAGCGCAGGTGAGCGGCCAGGGCGATAGCCTGCATGAACATATTGCGA | |
| | ACCTGGCGGGCAGCCCGGCGATTAAAAAAGGCATTCTGCAGACCGTGAAAGTGGTGGATGAACTGGTG | |
| | AAAGTGATGGGCCGCCATAAACCGGAAAACATTGTGATTGAAATGGCGCGCGAAAACCAGACCACCCA | |
| | GAAAGGCCAGAAAAACAGCCGCGAACGCATGAAACGCATTGAAGAAGGCATTAAAGAACTGGGCAGCC | |
| | AGATTCTGAAAGAACATCCGGTGGAAAACACCCAGCTGCAGAACGAAAAACTGTATCTGTATTATCTG | |
| | CAGAACGGCCGCGATATGTATGTGGATCAGGAACTGGATATTAACCGCCTGAGCGATTATGATGTGGA | |
| | TCATATTGTGCCGCAGAGCTTTCTGAAAGATGATAGCATTGATAACAAAGTGCTGACCCGCAGCGATA | |
| | AAAACCGCGGCAAAAGCGATAACGTGCCGAGCGAAGAAGTGGTGAAAAAAATGAAAAACTATTGGCGC | |
| | CAGCTGCTGAACGCGAAACTGATTACCCAGCGCAAATTTGATAACCTGACCAAAGCGGAACGCGGCGG | |
| | CCTGAGCGAACTGGATAAAGCGGGCTTTATTAAACGCCAGCTGGTGGAAACCCGCCAGATTACCAAAC | |
| | ATGTGGCGCAGATTCTGGATAGCCGCATGAACACCAAATATGATGAAAACGATAAACTGATTCGCGAA | |
| | GTGAAAGTGATTACCCTGAAAAGCAAACTGGTGAGCGATTTTCGCAAAGATTTTCAGTTTTATAAAGT | |
| | GCGCGAAATTAACAACTATCATCATGCGCATGATGCGTATCTGAACGCGGTGGTGGGCACCGCGCTGA | |
| | TTAAAAAATATCCGAAACTGGAAAGCGAATTTGTGTATGGCGATTATAAAGTGTATGATGTGCGCAAA | |
| | ATGATTGCGAAAAGCGAACAGGAAATTGGCAAAGCGACCGCGAAATATTTTTTTTATAGCAACATTAT | |
| | GAACTTTTTTAAAACCGAAATTACCCTGGCGAACGGCGAAATTCGCAAACGCCCGCTGATTGAAACCA | |
| | ACGGCGAAACCGGCGAAATTGTGTGGGATAAAGGCCGCGATTTTGCGACCGTGCGCAAAGTGCTGAGC | |
| | ATGCCGCAGGTGAACATTGTGAAAAAAACCGAAGTGCAGACCGGCGGCTTTAGCAAAGAAAGCATTCT | |
| | GCCGAAACGCAACAGCGATAAACTGATTGCGCGCAAAAAAGATTGGGATCCGAAAAAATATGGCGGCT | |
| | TTGATAGCCCGACCGTGGCGTATAGCGTGCTGGTGGTGGCGAAAGTGGAAAAAGGCAAAAGCAAAAAA | |
| | CTGAAAAGCGTGAAAGAACTGCTGGGCATTACCATTATGGAACGCAGCAGCTTTGAAAAAAACCCGAT | |
| | TGATTTTCTGGAAGCGAAAGGCTATAAAGAAGTGAAAAAAGATCTGATTATTAAACTGCCGAAATATA | |
| | GCCTGTTTGAACTGGAAAACGGCCGCAAACGCATGCTGGCGAGCGCGGGCGAACTGCAGAAAGGCAAC | |
| | GAACTGGCGCTGCCGAGCAAATATGTGAACTTTCTGTATCTGGCGAGCCATTATGAAAAACTGAAAGG | |
| | CAGCCCGGAAGATAACGAACAGAAACAGCTGTTTGTGGAACAGCATAAACATTATCTGGATGAAATTA | |
| | TTGAACAGATTAGCGAATTTAGCAAACGCGTGATTCTGGCGGATGCGAACCTGGATAAAGTGCTGAGC | |
| | GCGTATAACAAACATCGCGATAAACCGATTCGCGAACAGGCGGAAAACATTATTCATCTGTTTACCCT | |
| | GACCAACCTGGGCGCGCCGGCGGCGTTTAAATATTTTGATACCACCATTGATCGCAAACGCTATACCA | |
| | GCACCAAAGAAGTGCTGGATGCGACCCTGATTCATCAGAGCATTACCGGCCTGTATGAAACCCGCATT | |
| | GATCTGAGCCAGCTGGGCGGCGAT | |
| SpCas9 | ATGGATAAGAAATACTCAATAGGCTTAGATATCGGCACAAATAGCGTCGGATGGGCGGTGATCACTGA | 7 |
| Streptococcus | TGATTATAAGGTTCCGTCTAAAAAGTTCAAGGTTCTGGGAAATACAGACCGCCACAGTATCAAAAAAA | |
| pyogenes | ATCTTATAGGGGCTCTTTTATTTGGCAGTGGAGAGACAGCGGAAGCGACTCGTCTCAAACGGACAGCT | |
| MGAS1882 wild | CGTAGAAGGTATACACGTCGGAAGAATCGTATTTGTTATCTACAGGAGATTTTTTCAAATGAGATGGC | |
| type | GAAAGTAGATGATAGTTTCTTTCATCGACTTGAAGAGTCTTTTTTGGTGGAAGAAGACAAGAAGCATG | |
| NC_017053.1 | AACGTCATCCTATTTTTGGAAATATAGTAGATGAAGTTGCTTATCATGAGAAATATCCAACTATCTAT | |
| | CATCTGCGAAAAAAATTGGCAGATTCTACTGATAAAGCGGATTTGCGCTTAATCTATTTGGCCTTAGC | |
| | GCATATGATTAAGTTTCGTGGTCATTTTTTGATTGAGGGAGATTTAAATCCTGATAATAGTGATGTGG | |
| | ACAAACTATTTATCCAGTTGGTACAAATCTACAATCAATTATTTGAAGAAAACCCTATTAACGCAAGT | |
| | AGAGTAGATGCTAAAGCGATTCTTTCTGCACGATTGAGTAAATCAAGACGATTAGAAAATCTCATTGC | |
| | TCAGCTCCCCGGTGAGAAGAGAAATGGCTTGTTTGGGAATCTCATTGCTTTGTCATTGGGATTGACCC | |
| | CTAATTTTAAATCAAATTTTGATTTGGCAGAAGATGCTAAATTACAGCTTTCAAAAGATACTTACGAT | |
| | GATGATTTAGATAATTTATTGGCGCAAATTGGAGATCAATATGCTGATTTGTTTTTGGCAGCTAAGAA | |
| | TTTATCAGATGCTATTTTACTTTCAGATATCCTAAGAGTAAATAGTGAAATAACTAAGGCTCCCCTAT | |
| | CAGCTTCAATGATTAAGCGCTACGATGAACATCATCAAGACTTGACTCTTTTAAAAGCTTTAGTTCGA | |
| | CAACAACTTCCAGAAAAGTATAAAGAAATCTTTTTTGATCAATCAAAAAACGGATATGCAGGTTATAT | |
| | TGATGGGGGAGCTAGCCAAGAAGAATTTTATAAATTTATCAAACCAATTTTAGAAAAAATGGATGGTA | |
| | CTGAGGAATTATTGGTGAAACTAAATCGTGAAGATTTGCTGCGCAAGCAACGGACCTTTGACAACGGC | |
| | TCTATTCCCCATCAAATTCACTTGGGTGAGCTGCATGCTATTTTGAGAAGACAAGAAGACTTTTATCC | |
| | ATTTTTAAAAGACAATCGTGAGAAGATTGAAAAAATCTTGACTTTTCGAATTCCTTATTATGTTGGTC | |
| | CATTGGCGCGTGGCAATAGTCGTTTTGCATGGATGACTCGGAAGTCTGAAGAAACAATTACCCCATGG | |
| | AATTTTGAAGAAGTTGTCGATAAAGGTGCTTCAGCTCAATCATTTATTGAACGCATGACAAACTTTGA | |
| | TAAAAATCTTCCAAATGAAAAAGTACTACCAAAACATAGTTTGCTTTATGAGTATTTTACGGTTTATA | |
| | ACGAATTGACAAAGGTCAAATATGTTACTGAGGGAATGCGAAAACCAGCATTTCTTTCAGGTGAACAG | |
| | AAGAAAGCCATTGTTGATTTACTCTTCAAAACAAATCGAAAAGTAACCGTTAAGCAATTAAAAGAAGA | |
| | TTATTTCAAAAAAATAGAATGTTTTGATAGTGTTGAAATTTCAGGAGTTGAAGATAGATTTAATGCTT | |
| | CATTAGGCGCCTACCATGATTTGCTAAAAATTATTAAAGATAAAGATTTTTTGGATAATGAAGAAAAT | |
| | GAAGATATCTTAGAGGATATTGTTTTAACATTGACCTTATTTGAAGATAGGGGGATGATTGAGGAAAG | |
| | ACTTAAAACATATGCTCACCTCTTTGATGATAAGGTGATGAAACAGCTTAAACGTCGCCGTTATACTG | |
| | GTTGGGGACGTTTGTCTCGAAAATTGATTAATGGTATTAGGGATAAGCAATCTGGCAAAACAATATTA | |
| | GATTTTTTGAAATCAGATGGTTTTGCCAATCGCAATTTTATGCAGCTGATCCATGATGATAGTTTGAC | |
| | ATTTAAAGAAGATATTCAAAAAGCACAGGTGTCTGGACAAGGCCATAGTTTACATGAACAGATTGCTA | |
| | ACTTAGCTGGCAGTCCTGCTATTAAAAAAGGTATTTTACAGACTGTAAAAATTGTTGATGAACTGGTC | |
| | AAAGTAATGGGGCATAAGCCAGAAAATATCGTTATTGAAATGGCACGTGAAAATCAGACAACTCAAAA | |
| | GGGCCAGAAAAATTCGCGAGAGCGTATGAAACGAATCGAAGAAGGTATCAAAGAATTAGGAAGTCAGA | |
| | TTCTTAAAGAGCATCCTGTTGAAAATACTCAATTGCAAAATGAAAAGCTCTATCTCTATTATCTACAA | |
| | AATGGAAGAGACATGTATGTGGACCAAGAATTAGATATTAATCGTTTAAGTGATTATGATGTCGATCA | |
| | CATTGTTCCACAAAGTTTCATTAAAGACGATTCAATAGACAATAAGGTACTAACGCGTTCTGATAAAA | |
| | ATCGTGGTAAATCGGATAACGTTCCAAGTGAAGAAGTAGTCAAAAAGATGAAAAACTATTGGAGACAA | |
| | CTTCTAAACGCCAAGTTAATCACTCAACGTAAGTTTGATAATTTAACGAAAGCTGAACGTGGAGGTTT | |
| | GAGTGAACTTGATAAAGCTGGTTTTATCAAACGCCAATTGGTTGAAACTCGCCAAATCACTAAGCATG | |
| | TGGCACAAATTTTGGATAGTCGCATGAATACTAAATACGATGAAAATGATAAACTTATTCGAGAGGTT | |
| | AAAGTGATTACCTTAAAATCTAAATTAGTTTCTGACTTCCGAAAAGATTTCCAATTCTATAAAGTACG | |
| | TGAGATTAACAATTACCATCATGCCCATGATGCGTATCTAAATGCCGTCGTTGGAACTGCTTTGATTA | |
| | AGAAATATCCAAAACTTGAATCGGAGTTTGTCTATGGTGATTATAAAGTTTATGATGTTCGTAAAATG | |
| | ATTGCTAAGTCTGAGCAAGAAATAGGCAAAGCAACCGCAAAATATTTCTTTTACTCTAATATCATGAA | |
| | CTTCTTCAAAACAGAAATTACACTTGCAAATGGAGAGATTCGCAAACGCCCTCTAATCGAAACTAATG | |
| | GGGAAACTGGAGAAATTGTCTGGGATAAAGGGCGAGATTTTGCCACAGTGCGCAAAGTATTGTCCATG | |
| | CCCCAAGTCAATATTGTCAAGAAAACAGAAGTACAGACAGGCGGATTCTCCAAGGAGTCAATTTTACC | |
| | AAAAAGAAATTCGGACAAGCTTATTGCTCGTAAAAAAGACTGGGATCCAAAAAAATATGGTGGTTTTG | |
| | ATAGTCCAACGGTAGCTTATTCAGTCCTAGTGGTTGCTAAGGTGGAAAAAGGGAAATCGAAGAAGTTA | |
| | AAATCCGTTAAAGAGTTACTAGGGATCACAATTATGGAAAGAAGTTCCTTTGAAAAAAATCCGATTGA | |
| | CTTTTTAGAAGCTAAAGGATATAAGGAAGTTAAAAAAGACTTAATCATTAAACTACCTAAATATAGTC | |
| | TTTTTGAGTTAGAAAACGGTCGTAAACGGATGCTGGCTAGTGCCGGAGAATTACAAAAAGGAAATGAG | |
| | CTGGCTCTGCCAAGCAAATATGTGAATTTTTTATATTTAGCTAGTCATTATGAAAAGTTGAAGGGTAG | |
| | TCCAGAAGATAACGAACAAAAACAATTGTTTGTGGAGCAGCATAAGCATTATTTAGATGAGATTATTG | |
| | AGCAAATCAGTGAATTTTCTAAGCGTGTTATTTTAGCAGATGCCAATTTAGATAAAGTTCTTAGTGCA | |
| | TATAACAAACATAGAGACAAACCAATACGTGAACAAGCAGAAAATATTATTCATTTATTTACGTTGAC | |
| | GAATCTTGGAGCTCCCGCTGCTTTTAAATATTTTGATACAACAATTGATCGTAAACGATATACGTCTA | |
| | CAAAAGAAGTTTTAGATGCCACTCTTATCCATCAATCCATCACTGGTCTTTATGAAACACGCATTGAT | |
| | TTGAGTCAGCTAGGAGGTGACTGA | |
| SpCas9 | MDKKYSIGLDIGTNSVGWAVITDDYKVPSKKFKVLGNTDRHSIKKNLIGALLFGSGETAEATRLKRTA | 8 |
| Streptococcus | RRRYTRRKNRICYLQEIFSNEMAKVDDSFFHRLEESFLVEEDKKHERHPIFGNIVDEVAYHEKYPTIY | |
| pyogenes | HLRKKLADSTDKADLRLIYLALAHMIKFRGHFLIEGDLNPDNSDVDKLFIQLVQIYNQLFEENPINAS | |
| MGAS1882 wild | RVDAKAILSARLSKSRRLENLIAQLPGEKRNGLFGNLIALSLGLTPNFKSNFDLAEDAKLQLSKDTYD | |
| type | DDLDNLLAQIGDQYADLFLAAKNLSDAILLSDILRVNSEITKAPLSASMIKRYDEHHQDLTLLKALVR | |
| NC_017053.1 | QQLPEKYKEIFFDQSKNGYAGYIDGGASQEEFYKFIKPILEKMDGTEELLVKLNREDLLRKQRTFDNG | |
| | SIPHQIHLGELHAILRRQEDFYPFLKDNREKIEKILTFRIPYYVGPLARGNSRFAWMTRKSEETITPW | |
| | NFEEVVDKGASAQSFIERMTNFDKNLPNEKVLPKHSLLYEYFTVYNELTKVKYVTEGMRKPAFLSGEQ | |
| | KKAIVDLLFKTNRKVTVKQLKEDYFKKIECFDSVEISGVEDRFNASLGAYHDLLKIIKDKDFLDNEEN | |
| | EDILEDIVLTLTLFEDRGMIEERLKTYAHLFDDKVMKQLKRRRYTGWGRLSRKLINGIRDKQSGKTIL | |
| | DFLKSDGFANRNFMQLIHDDSLTFKEDIQKAQVSGQGHSLHEQIANLAGSPAIKKGILQTVKIVDELV | |
| | KVMGHKPENIVIEMARENQTTQKGQKNSRERMKRIEEGIKELGSQILKEHPVENTQLQNEKLYLYYLQ | |
| | NGRDMYVDQELDINRLSDYDVDHIVPQSFIKDDSIDNKVLTRSDKNRGKSDNVPSEEVVKKMKNYWRQ | |
| | LLNAKLITQRKFDNLTKAERGGLSELDKAGFIKRQLVETRQITKHVAQILDSRMNTKYDENDKLIREV | |
| | KVITLKSKLVSDFRKDFQFYKVREINNYHHAHDAYLNAVVGTALIKKYPKLESEFVYGDYKVYDVRKM | |
| | IAKSEQEIGKATAKYFFYSNIMNFFKTEITLANGEIRKRPLIETNGETGEIVWDKGRDFATVRKVLSM | |
| | PQVNIVKKTEVQTGGFSKESILPKRNSDKLIARKKDWDPKKYGGFDSPTVAYSVLVVAKVEKGKSKKL | |
| | KSVKELLGITIMERSSFEKNPIDFLEAKGYKEVKKDLIIKLPKYSLFELENGRKRMLASAGELQKGNE | |
| | LALPSKYVNFLYLASHYEKLKGSPEDNEQKQLFVEQHKHYLDEIIEQISEFSKRVILADANLDKVLSA | |
| | YNKHRDKPIREQAENIIHLFTLTNLGAPAAFKYFDTTIDRKRYTSTKEVLDATLIHQSITGLYETRID | |
| | LSQLGGD | |
| SpCas9 | ATGGATAAAAAGTATTCTATTGGTTTAGACATCGGCACTAATTCCGTTGGATGGGCTGTCATAACCGA | 9 |
| Streptococcus | TGAATACAAAGTACCTTCAAAGAAATTTAAGGTGTTGGGGAACACAGACCGTCATTCGATTAAAAAGA | |
| pyogenes wild | ATCTTATCGGTGCCCTCCTATTCGATAGTGGCGAAACGGCAGAGGCGACTCGCCTGAAACGAACCGCT | |
| type | CGGAGAAGGTATACACGTCGCAAGAACCGAATATGTTACTTACAAGAAATTTTTAGCAATGAGATGGC | |
| SWBC2D7W014 | CAAAGTTGACGATTCTTTCTTTCACCGTTTGGAAGAGTCCTTCCTTGTCGAAGAGGACAAGAAACATG | |
| | AACGGCACCCCATCTTTGGAAACATAGTAGATGAGGTGGCATATCATGAAAAGTACCCAACGATTTAT | |
| | CACCTCAGAAAAAAGCTAGTTGACTCAACTGATAAAGCGGACCTGAGGTTAATCTACTTGGCTCTTGC | |
| | CCATATGATAAAGTTCCGTGGGCACTTTCTCATTGAGGGTGATCTAAATCCGGACAACTCGGATGTCG | |
| | ACAAACTGTTCATCCAGTTAGTACAAACCTATAATCAGTTGTTTGAAGAGAACCCTATAAATGCAAGT | |
| | GGCGTGGATGCGAAGGCTATTCTTAGCGCCCGCCTCTCTAAATCCCGACGGCTAGAAAACCTGATCGC | |
| | ACAATTACCCGGAGAGAAGAAAAATGGGTTGTTCGGTAACCTTATAGCGCTCTCACTAGGCCTGACAC | |
| | CAAATTTTAAGTCGAACTTCGACTTAGCTGAAGATGCCAAATTGCAGCTTAGTAAGGACACGTACGAT | |
| | GACGATCTCGACAATCTACTGGCACAAATTGGAGATCAGTATGCGGACTTATTTTTGGCTGCCAAAAA | |
| | CCTTAGCGATGCAATCCTCCTATCTGACATACTGAGAGTTAATACTGAGATTACCAAGGCGCCGTTAT | |
| | CCGCTTCAATGATCAAAAGGTACGATGAACATCACCAAGACTTGACACTTCTCAAGGCCCTAGTCCGT | |
| | CAGCAACTGCCTGAGAAATATAAGGAAATATTCTTTGATCAGTCGAAAAACGGGTACGCAGGTTATAT | |
| | TGACGGCGGAGCGAGTCAAGAGGAATTCTACAAGTTTATCAAACCCATATTAGAGAAGATGGATGGGA | |
| | CGGAAGAGTTGCTTGTAAAACTCAATCGCGAAGATCTACTGCGAAAGCAGCGGACTTTCGACAACGGT | |
| | AGCATTCCACATCAAATCCACTTAGGCGAATTGCATGCTATACTTAGAAGGCAGGAGGATTTTTATCC | |
| | GTTCCTCAAAGACAATCGTGAAAAGATTGAGAAAATCCTAACCTTTCGCATACCTTACTATGTGGGAC | |
| | CCCTGGCCCGAGGGAACTCTCGGTTCGCATGGATGACAAGAAAGTCCGAAGAAACGATTACTCCATGG | |
| | AATTTTGAGGAAGTTGTCGATAAAGGTGCGTCAGCTCAATCGTTCATCGAGAGGATGACCAACTTTGA | |
| | CAAGAATTTACCGAACGAAAAAGTATTGCCTAAGCACAGTTTACTTTACGAGTATTTCACAGTGTACA | |
| | ATGAACTCACGAAAGTTAAGTATGTCACTGAGGGCATGCGTAAACCCGCCTTTCTAAGCGGAGAACAG | |
| | AAGAAAGCAATAGTAGATCTGTTATTCAAGACCAACCGCAAAGTGACAGTTAAGCAATTGAAAGAGGA | |
| | CTACTTTAAGAAAATTGAATGCTTCGATTCTGTCGAGATCTCCGGGGTAGAAGATCGATTTAATGCGT | |
| | CACTTGGTACGTATCATGACCTCCTAAAGATAATTAAAGATAAGGACTTCCTGGATAACGAAGAGAAT | |
| | GAAGATATCTTAGAAGATATAGTGTTGACTCTTACCCTCTTTGAAGATCGGGAAATGATTGAGGAAAG | |
| | ACTAAAAACATACGCTCACCTGTTCGACGATAAGGTTATGAAACAGTTAAAGAGGCGTCGCTATACGG | |
| | GCTGGGGACGATTGTCGCGGAAACTTATCAACGGGATAAGAGACAAGCAAAGTGGTAAAACTATTCTC | |
| | GATTTTCTAAAGAGCGACGGCTTCGCCAATAGGAACTTTATGCAGCTGATCCATGATGACTCTTTAAC | |
| | CTTCAAAGAGGATATACAAAAGGCACAGGTTTCCGGACAAGGGGACTCATTGCACGAACATATTGCGA | |
| | ATCTTGCTGGTTCGCCAGCCATCAAAAAGGGCATACTCCAGACAGTCAAAGTAGTGGATGAGCTAGTT | |
| | AAGGTCATGGGACGTCACAAACCGGAAAACATTGTAATCGAGATGGCACGCGAAAATCAAACGACTCA | |
| | GAAGGGGCAAAAAAACAGTCGAGAGCGGATGAAGAGAATAGAAGAGGGTATTAAAGAACTGGGCAGCC | |
| | AGATCTTAAAGGAGCATCCTGTGGAAAATACCCAATTGCAGAACGAGAAACTTTACCTCTATTACCTA | |
| | CAAAATGGAAGGGACATGTATGTTGATCAGGAACTGGACATAAACCGTTTATCTGATTACGACGTCGA | |
| | TCACATTGTACCCCAATCCTTTTTGAAGGACGATTCAATCGACAATAAAGTGCTTACACGCTCGGATA | |
| | AGAACCGAGGGAAAAGTGACAATGTTCCAAGCGAGGAAGTCGTAAAGAAAATGAAGAACTATTGGCGG | |
| | CAGCTCCTAAATGCGAAACTGATAACGCAAAGAAAGTTCGATAACTTAACTAAAGCTGAGAGGGGTGG | |
| | CTTGTCTGAACTTGACAAGGCCGGATTTATTAAACGTCAGCTCGTGGAAACCCGCCAAATCACAAAGC | |
| | ATGTTGCACAGATACTAGATTCCCGAATGAATACGAAATACGACGAGAACGATAAGCTGATTCGGGAA | |
| | GTCAAAGTAATCACTTTAAAGTCAAAATTGGTGTCGGACTTCAGAAAGGATTTTCAATTCTATAAAGT | |
| | TAGGGAGATAAATAACTACCACCATGCGCACGACGCTTATCTTAATGCCGTCGTAGGGACCGCACTCA | |
| | TTAAGAAATACCCGAAGCTAGAAAGTGAGTTTGTGTATGGTGATTACAAAGTTTATGACGTCCGTAAG | |
| | ATGATCGCGAAAAGCGAACAGGAGATAGGCAAGGCTACAGCCAAATACTTCTTTTATTCTAACATTAT | |
| | GAATTTCTTTAAGACGGAAATCACTCTGGCAAACGGAGAGATACGCAAACGACCTTTAATTGAAACCA | |
| | ATGGGGAGACAGGTGAAATCGTATGGGATAAGGGCCGGGACTTCGCGACGGTGAGAAAAGTTTTGTCC | |
| | ATGCCCCAAGTCAACATAGTAAAGAAAACTGAGGTGCAGACCGGAGGGTTTTCAAAGGAATCGATTCT | |
| | TCCAAAAAGGAATAGTGATAAGCTCATCGCTCGTAAAAAGGACTGGGACCCGAAAAAGTACGGTGGCT | |
| | TCGATAGCCCTACAGTTGCCTATTCTGTCCTAGTAGTGGCAAAAGTTGAGAAGGGAAAATCCAAGAAA | |
| | CTGAAGTCAGTCAAAGAATTATTGGGGATAACGATTATGGAGCGCTCGTCTTTTGAAAAGAACCCCAT | |
| | CGACTTCCTTGAGGCGAAAGGTTACAAGGAAGTAAAAAAGGATCTCATAATTAAACTACCAAAGTATA | |
| | GTCTGTTTGAGTTAGAAAATGGCCGAAAACGGATGTTGGCTAGCGCCGGAGAGCTTCAAAAGGGGAAC | |
| | GAACTCGCACTACCGTCTAAATACGTGAATTTCCTGTATTTAGCGTCCCATTACGAGAAGTTGAAAGG | |
| | TTCACCTGAAGATAACGAACAGAAGCAACTTTTTGTTGAGCAGCACAAACATTATCTCGACGAAATCA | |
| | TAGAGCAAATTTCGGAATTCAGTAAGAGAGTCATCCTAGCTGATGCCAATCTGGACAAAGTATTAAGC | |
| | GCATACAACAAGCACAGGGATAAACCCATACGTGAGCAGGCGGAAAATATTATCCATTTGTTTACTCT | |
| | TACCAACCTCGGCGCTCCAGCCGCATTCAAGTATTTTGACACAACGATAGATCGCAAACGATACACTT | |
| | CTACCAAGGAGGTGCTAGACGCGACACTGATTCACCAATCCATCACGGGATTATATGAAACTCGGATA | |
| | GATTTGTCACAGCTTGGGGGTGACGGATCCCCCAAGAAGAAGAGGAAAGTCTCGAGCGACTACAAAGA | |
| | CCATGACGGTGATTATAAAGATCATGACATCGATTACAAGGATGACGATGACAAGGCTGCAGGA | |
| SpCas9 | MDKKYSIGLDIGTNSVGWAVITDEYKVPSKKFKVLGNTDRHSIKKNLIGALLFDSGETAEATRLKRTA | 10 |
| Streptococcus | RRRYTRRKNRICYLQEIFSNEMAKVDDSFFHRLEESFLVEEDKKHERHPIFGNIVDEVAYHEKYPTIY | |
| pyogenes wild | HLRKKLVDSTDKADLRLIYLALAHMIKFRGHFLIEGDLNPDNSDVDKLFIQLVQTYNQLFEENPINAS | |
| type | GVDAKAILSARLSKSRRLENLIAQLPGEKKNGLFGNLIALSLGLTPNFKSNFDLAEDAKLQLSKDTYD | |
| Encoded | DDLDNLLAQIGDQYADLFLAAKNLSDAILLSDILRVNTEITKAPLSASMIKRYDEHHQDLTLLKALVR | |
| product of | QQLPEKYKEIFFDQSKNGYAGYIDGGASQEEFYKFIKPILEKMDGTEELLVKLNREDLLRKQRTFDNG | |
| SWBC2D7W014 | SIPHQIHLGELHAILRRQEDFYPFLKDNREKIEKILTFRIPYYVGPLARGNSRFAWMTRKSEETITPW | |
| | NFEEVVDKGASAQSFIERMTNFDKNLPNEKVLPKHSLLYEYFTVYNELTKVKYVTEGMRKPAFLSGEQ | |
| | KKAIVDLLFKTNRKVTVKQLKEDYFKKIECFDSVEISGVEDRFNASLGTYHDLLKIIKDKDFLDNEEN | |
| | EDILEDIVLTLTLFEDREMIEERLKTYAHLFDDKVMKQLKRRRYTGWGRLSRKLINGIRDKQSGKTIL | |
| | DFLKSDGFANRNFMQLIHDDSLTFKEDIQKAQVSGQGDSLHEHIANLAGSPAIKKGILQTVKVVDELV | |
| | KVMGRHKPENIVIEMARENQTTQKGQKNSRERMKRIEEGIKELGSQILKEHPVENTQLQNEKLYLYYL | |
| | QNGRDMYVDQELDINRLSDYDVDHIVPQSFLKDDSIDNKVLTRSDKNRGKSDNVPSEEVVKKMKNYWR | |
| | QLLNAKLITQRKFDNLTKAERGGLSELDKAGFIKRQLVETRQITKHVAQILDSRMNTKYDENDKLIRE | |
| | VKVITLKSKLVSDFRKDFQFYKVREINNYHHAHDAYLNAVVGTALIKKYPKLESEFVYGDYKVYDVRK | |
| | MIAKSEQEIGKATAKYFFYSNIMNFFKTEITLANGEIRKRPLIETNGETGEIVWDKGRDFATVRKVLS | |
| | MPQVNIVKKTEVQTGGFSKESILPKRNSDKLIARKKDWDPKKYGGFDSPTVAYSVLVVAKVEKGKSKK | |
| | LKSVKELLGITIMERSSFEKNPIDFLEAKGYKEVKKDLIIKLPKYSLFELENGRKRMLASAGELQKGN | |
| | ELALPSKYVNFLYLASHYEKLKGSPEDNEQKQLFVEQHKHYLDEIIEQISEFSKRVILADANLDKVLS | |
| | AYNKHRDKPIREQAENIIHLFTLTNLGAPAAFKYFDTTIDRKRYTSTKEVLDATLIHQSITGLYETRI | |
| | DLSQLGGDGSPKKKRKVSSDYKDHDGDYKDHDIDYKDDDDKAAG | |
| SpCas9 | ATGGATAAGAAATACTCAATAGGCTTAGATATCGGCACAAATAGCGTCGGATGGGCGGTGATCACTGA | 11 |
| Streptococcus | TGAATATAAGGTTCCGTCTAAAAAGTTCAAGGTTCTGGGAAATACAGACCGCCACAGTATCAAAAAAA | |
| pyogenes M1GAS | ATCTTATAGGGGCTCTTTTATTTGACAGTGGAGAGACAGCGGAAGCGACTCGTCTCAAACGGACAGCT | |
| wild type | CGTAGAAGGTATACACGTCGGAAGAATCGTATTTGTTATCTACAGGAGATTTTTTCAAATGAGATGGC | |
| NC_002737.2 | GAAAGTAGATGATAGTTTCTTTCATCGACTTGAAGAGTCTTTTTTGGTGGAAGAAGACAAGAAGCATG | |
| | AACGTCATCCTATTTTTGGAAATATAGTAGATGAAGTTGCTTATCATGAGAAATATCCAACTATCTAT | |
| | CATCTGCGAAAAAAATTGGTAGATTCTACTGATAAAGCGGATTTGCGCTTAATCTATTTGGCCTTAGC | |
| | GCATATGATTAAGTTTCGTGGTCATTTTTTGATTGAGGGAGATTTAAATCCTGATAATAGTGATGTGG | |
| | ACAAACTATTTATCCAGTTGGTACAAACCTACAATCAATTATTTGAAGAAAACCCTATTAACGCAAGT | |
| | GGAGTAGATGCTAAAGCGATTCTTTCTGCACGATTGAGTAAATCAAGACGATTAGAAAATCTCATTGC | |
| | TCAGCTCCCCGGTGAGAAGAAAAATGGCTTATTTGGGAATCTCATTGCTTTGTCATTGGGTTTGACCC | |
| | CTAATTTTAAATCAAATTTTGATTTGGCAGAAGATGCTAAATTACAGCTTTCAAAAGATACTTACGAT | |
| | GATGATTTAGATAATTTATTGGCGCAAATTGGAGATCAATATGCTGATTTGTTTTTGGCAGCTAAGAA | |
| | TTTATCAGATGCTATTTTACTTTCAGATATCCTAAGAGTAAATACTGAAATAACTAAGGCTCCCCTAT | |
| | CAGCTTCAATGATTAAACGCTACGATGAACATCATCAAGACTTGACTCTTTTAAAAGCTTTAGTTCGA | |
| | CAACAACTTCCAGAAAAGTATAAAGAAATCTTTTTTGATCAATCAAAAAACGGATATGCAGGTTATAT | |
| | TGATGGGGGAGCTAGCCAAGAAGAATTTTATAAATTTATCAAACCAATTTTAGAAAAAATGGATGGTA | |
| | CTGAGGAATTATTGGTGAAACTAAATCGTGAAGATTTGCTGCGCAAGCAACGGACCTTTGACAACGGC | |
| | TCTATTCCCCATCAAATTCACTTGGGTGAGCTGCATGCTATTTTGAGAAGACAAGAAGACTTTTATCC | |
| | ATTTTTAAAAGACAATCGTGAGAAGATTGAAAAAATCTTGACTTTTCGAATTCCTTATTATGTTGGTC | |
| | CATTGGCGCGTGGCAATAGTCGTTTTGCATGGATGACTCGGAAGTCTGAAGAAACAATTACCCCATGG | |
| | AATTTTGAAGAAGTTGTCGATAAAGGTGCTTCAGCTCAATCATTTATTGAACGCATGACAAACTTTGA | |
| | TAAAAATCTTCCAAATGAAAAAGTACTACCAAAACATAGTTTGCTTTATGAGTATTTTACGGTTTATA | |
| | ACGAATTGACAAAGGTCAAATATGTTACTGAAGGAATGCGAAAACCAGCATTTCTTTCAGGTGAACAG | |
| | AAGAAAGCCATTGTTGATTTACTCTTCAAAACAAATCGAAAAGTAACCGTTAAGCAATTAAAAGAAGA | |
| | TTATTTCAAAAAAATAGAATGTTTTGATAGTGTTGAAATTTCAGGAGTTGAAGATAGATTTAATGCTT | |
| | CATTAGGTACCTACCATGATTTGCTAAAAATTATTAAAGATAAAGATTTTTTGGATAATGAAGAAAAT | |
| | GAAGATATCTTAGAGGATATTGTTTTAACATTGACCTTATTTGAAGATAGGGAGATGATTGAGGAAAG | |
| | ACTTAAAACATATGCTCACCTCTTTGATGATAAGGTGATGAAACAGCTTAAACGTCGCCGTTATACTG | |
| | GTTGGGGACGTTTGTCTCGAAAATTGATTAATGGTATTAGGGATAAGCAATCTGGCAAAACAATATTA | |
| | GATTTTTTGAAATCAGATGGTTTTGCCAATCGCAATTTTATGCAGCTGATCCATGATGATAGTTTGAC | |
| | ATTTAAAGAAGACATTCAAAAAGCACAAGTGTCTGGACAAGGCGATAGTTTACATGAACATATTGCAA | |
| | ATTTAGCTGGTAGCCCTGCTATTAAAAAAGGTATTTTACAGACTGTAAAAGTTGTTGATGAATTGGTC | |
| | AAAGTAATGGGGCGGCATAAGCCAGAAAATATCGTTATTGAAATGGCACGTGAAAATCAGACAACTCA | |
| | AAAGGGCCAGAAAAATTCGCGAGAGCGTATGAAACGAATCGAAGAAGGTATCAAAGAATTAGGAAGTC | |
| | AGATTCTTAAAGAGCATCCTGTTGAAAATACTCAATTGCAAAATGAAAAGCTCTATCTCTATTATCTC | |
| | CAAAATGGAAGAGACATGTATGTGGACCAAGAATTAGATATTAATCGTTTAAGTGATTATGATGTCGA | |
| | TCACATTGTTCCACAAAGTTTCCTTAAAGACGATTCAATAGACAATAAGGTCTTAACGCGTTCTGATA | |
| | AAAATCGTGGTAAATCGGATAACGTTCCAAGTGAAGAAGTAGTCAAAAAGATGAAAAACTATTGGAGA | |
| | CAACTTCTAAACGCCAAGTTAATCACTCAACGTAAGTTTGATAATTTAACGAAAGCTGAACGTGGAGG | |
| | TTTGAGTGAACTTGATAAAGCTGGTTTTATCAAACGCCAATTGGTTGAAACTCGCCAAATCACTAAGC | |
| | ATGTGGCACAAATTTTGGATAGTCGCATGAATACTAAATACGATGAAAATGATAAACTTATTCGAGAG | |
| | GTTAAAGTGATTACCTTAAAATCTAAATTAGTTTCTGACTTCCGAAAAGATTTCCAATTCTATAAAGT | |
| | ACGTGAGATTAACAATTACCATCATGCCCATGATGCGTATCTAAATGCCGTCGTTGGAACTGCTTTGA | |
| | TTAAGAAATATCCAAAACTTGAATCGGAGTTTGTCTATGGTGATTATAAAGTTTATGATGTTCGTAAA | |
| | ATGATTGCTAAGTCTGAGCAAGAAATAGGCAAAGCAACCGCAAAATATTTCTTTTACTCTAATATCAT | |
| | GAACTTCTTCAAAACAGAAATTACACTTGCAAATGGAGAGATTCGCAAACGCCCTCTAATCGAAACTA | |
| | ATGGGGAAACTGGAGAAATTGTCTGGGATAAAGGGCGAGATTTTGCCACAGTGCGCAAAGTATTGTCC | |
| | ATGCCCCAAGTCAATATTGTCAAGAAAACAGAAGTACAGACAGGCGGATTCTCCAAGGAGTCAATTTT | |
| | ACCAAAAAGAAATTCGGACAAGCTTATTGCTCGTAAAAAAGACTGGGATCCAAAAAAATATGGTGGTT | |
| | TTGATAGTCCAACGGTAGCTTATTCAGTCCTAGTGGTTGCTAAGGTGGAAAAAGGGAAATCGAAGAAG | |
| | TTAAAATCCGTTAAAGAGTTACTAGGGATCACAATTATGGAAAGAAGTTCCTTTGAAAAAAATCCGAT | |
| | TGACTTTTTAGAAGCTAAAGGATATAAGGAAGTTAAAAAAGACTTAATCATTAAACTACCTAAATATA | |
| | GTCTTTTTGAGTTAGAAAACGGTCGTAAACGGATGCTGGCTAGTGCCGGAGAATTACAAAAAGGAAAT | |
| | GAGCTGGCTCTGCCAAGCAAATATGTGAATTTTTTATATTTAGCTAGTCATTATGAAAAGTTGAAGGG | |
| | TAGTCCAGAAGATAACGAACAAAAACAATTGTTTGTGGAGCAGCATAAGCATTATTTAGATGAGATTA | |
| | TTGAGCAAATCAGTGAATTTTCTAAGCGTGTTATTTTAGCAGATGCCAATTTAGATAAAGTTCTTAGT | |
| | GCATATAACAAACATAGAGACAAACCAATACGTGAACAAGCAGAAAATATTATTCATTTATTTACGTT | |
| | GACGAATCTTGGAGCTCCCGCTGCTTTTAAATATTTTGATACAACAATTGATCGTAAACGATATACGT | |
| | CTACAAAAGAAGTTTTAGATGCCACTCTTATCCATCAATCCATCACTGGTCTTTATGAAACACGCATT | |
| | GATTTGAGTCAGCTAGGAGGTGACTGA | |
| SpCas9 | MDKKYSIGLDIGTNSVGWAVITDEYKVPSKKFKVLGNTDRHSIKKNLIGALLFDSGETAEATRLKRTA | 12 |
| Streptococcus | RRRYTRRKNRICYLQEIFSNEMAKVDDSFFHRLEESFLVEEDKKHERHPIFGNIVDEVAYHEKYPTIY | |
| pyogenes M1GAS | HLRKKLVDSTDKADLRLIYLALAHMIKFRGHFLIEGDLNPDNSDVDKLFIQLVQTYNQLFEENPINAS | |
| wild type | GVDAKAILSARLSKSRRLENLIAQLPGEKKNGLFGNLIALSLGLTPNFKSNFDLAEDAKLQLSKDTYD | |
| Encoded | DDLDNLLAQIGDQYADLFLAAKNLSDAILLSDILRVNTEITKAPLSASMIKRYDEHHQDLTLLKALVR | |
| product of | QQLPEKYKEIFFDQSKNGYAGYIDGGASQEEFYKFIKPILEKMDGTEELLVKLNREDLLRKQRTFDNG | |
| NC_002737.2 | SIPHQIHLGELHAILRRQEDFYPFLKDNREKIEKILTFRIPYYVGPLARGNSRFAWMTRKSEETITPW | |
| (100% | NFEEVVDKGASAQSFIERMTNFDKNLPNEKVLPKHSLLYEYFTVYNELTKVKYVTEGMRKPAFLSGEQ | |
| identical to | KKAIVDLLFKTNRKVTVKQLKEDYFKKIECFDSVEISGVEDRFNASLGTYHDLLKIIKDKDFLDNEEN | |
| the canonical | EDILEDIVLTLTLFEDREMIEERLKTYAHLFDDKVMKQLKRRRYTGWGRLSRKLINGIRDKQSGKTIL | |
| Q99ZW2 | DFLKSDGFANRNFMQLIHDDSLTFKEDIQKAQVSGQGDSLHEHIANLAGSPAIKKGILQTVKVVDELV | |
| wild type) | KVMGRHKPENIVIEMARENQTTQKGQKNSRERMKRIEEGIKELGSQILKEHPVENTQLQNEKLYLYYL | |
| | QNGRDMYVDQELDINRLSDYDVDHIVPQSFLKDDSIDNKVLTRSDKNRGKSDNVPSEEVVKKMKNYWR | |
| | QLLNAKLITQRKFDNLTKAERGGLSELDKAGFIKRQLVETRQITKHVAQILDSRMNTKYDENDKLIRE | |
| | VKVITLKSKLVSDFRKDFQFYKVREINNYHHAHDAYLNAVVGTALIKKYPKLESEFVYGDYKVYDVRK | |
| | MIAKSEQEIGKATAKYFFYSNIMNFFKTEITLANGEIRKRPLIETNGETGEIVWDKGRDFATVRKVLS | |
| | MPQVNIVKKTEVQTGGFSKESILPKRNSDKLIARKKDWDPKKYGGFDSPTVAYSVLVVAKVEKGKSKK | |
| | LKSVKELLGITIMERSSFEKNPIDFLEAKGYKEVKKDLIIKLPKYSLFELENGRKRMLASAGELQKGN | |
| | ELALPSKYVNFLYLASHYEKLKGSPEDNEQKQLFVEQHKHYLDEIIEQISEFSKRVILADANLDKVLS | |
| | AYNKHRDKPIREQAENIIHLFTLTNLGAPAAFKYFDTTIDRKRYTSTKEVLDATLIHQSITGLYETRI | |
| | DLSQLGGD | |
| Cas9 | DKKYSIGLAIGTNSVGWAVITDEYKVPSKKFKVLGNTDRHSIKKNLIGALLFDSGETAEATRLKRTAR | 407 |
| | RRYTRRKNRICYLQEIFSNEMAKVDDSFFHRLEESFLVEEDKKHERHPIFGNIVDEVAYHEKYPTIYH | |
| | LRKKLVDSTDKADLRLIYLALAHMIKFRGHFLIEGDLNPDNSDVDKLFIQLVQTYNQLFEENPINASG | |
| | VDAKAILSARLSKSRRLENLIAQLPGEKKNGLFGNLIALSLGLTPNFKSNFDLAEDAKLQLSKDTYDD | |
| | DLDNLLAQIGDQYADLFLAAKNLSDAILLSDILRVNTEITKAPLSASMIKRYDEHHQDLTLLKALVRQ | |
| | QLPEKYKEIFFDQSKNGYAGYIDGGASQEEFYKFIKPILEKMDGTEELLVKLNREDLLRKQRTFDNGS | |
| | IPHQIHLGELHAILRRQEDFYPFLKDNREKIEKILTFRIPYYVGPLARGNSRFAWMTRKSEETITPWN | |
| | FEEVVDKGASAQSFIERMTNFDKNLPNEKVLPKHSLLYEYFTVYNELTKVKYVTEGMRKPAFLSGEQK | |
| | KAIVDLLFKTNRKVTVKQLKEDYFKKIECFDSVEISGVEDRFNASLGTYHDLLKIIKDKDFLDNEENE | |
| | DILEDIVLTLTLFEDREMIEERLKTYAHLFDDKVMKQLKRRRYTGWGRLSRKLINGIRDKQSGKTILD | |
| | FLKSDGFANRNFMQLIHDDSLTFKEDIQKAQVSGQGDSLHEHIANLAGSPAIKKGILQTVKVVDELVK | |
| | VMGRHKPENIVIEMARENQTTQKGQKNSRERMKRIEEGIKELGSQILKEHPVENTQLQNEKLYLYYLQ | |
| | NGRDMYVDQELDINRLSDYDVDHIVPQSFLKDDSIDNKVLTRSDKNRGKSDNVPSEEVVKKMKNYWRQ | |
| | LLNAKLITQRKFDNLTKAERGGLSELDKAGFIKRQLVETRQITKHVAQILDSRMNTKYDENDKLIREV | |
| | KVITLKSKLVSDFRKDFQFYKVREINNYHHAHDAYLNAVVGTALIKKYPKLESEFVYGDYKVYDVRKM | |
| | IAKSEQEIGKATAKYFFYSNIMNFFKTEITLANGEIRKRPLIETNGETGEIVWDKGRDFATVRKVLSM | |
| | PQVNIVKKTEVQTGGFSKESILPKRNSDKLIARKKDWDPKKYGGFVSPTVAYSVLVVAKVEKGKSKKL | |
| | KSVKELLGITIMERSSFEKNPIDFLEAKGYKEVKKDLIIKLPKYSLFELENGRKRMLASARELQKGNE | |
| | LALPSKYVNFLYLASHYEKLKGSPEDNEQKQLFVEQHKHYLDEIIEQISEFSKRVILADANLDKVLSA | |
| | YNKHRDKPIREQAENIIHLFTLTNLGAPAAFKYFDTTIDRKQYRSTKEVLDATLIHQSITGLYETRID | |
| | LSQLGGD | |

Wild type Cas9 orthologs

| | | |
|---|---|---|
| LfCas 9 | MKEYHIGLDIGTSSIGWAVTDSQFKLMRIKGKTAIGVRLFEEGKTAAERRTFRTTRRRLKRRKWRLHY | 13 |
| Lactobacillus | LDEIFAPHLQEVDENFLRRLKQSNIHPEDPTKNQAFIGKLLFPDLLKKNERGYPTLIKMRDELPVEQR | |
| fermentum wild | AHYPVMNIYKLREAMINEDRQFDLREVYLAVHHIVKYRGHFLNNASVDKFKVGRIDFDKSFNVLNEAY | |
| type | EELQNGEGSFTIEPSKVEKIGQLLLDTKMRKLDRQKAVAKLLEVKVADKEETKRNKQIATAMSKLVLG | |
| GenBank: | YKADFATVAMANGNEWKIDLSSETSEDEIEKFREELSDAQNDILTEITSLFSQIMLNEIVPNGMSISE | |
| SNX31424.11 | SMMDRYWTHERQLAEVKEYLATQPASARKEFDQVYNKYIGQAPKERGFDLEKGLKKILSKKENWKEID | |
| | ELLKAGDFLPKQRTSANGVIPHQMHQQELDRIIEKQAKYYPWLATENPATGERDRHQAKYELDQLVSF | |
| | RIPYYVGPLVTPEVQKATSGAKFAWAKRKEDGEITPWNLWDKIDRAESAEAFIKRMTVKDTYLLNEDV | |
| | LPANSLLYQKYNVLNELNNVRVNGRRLSVGIKQDIYTELFKKKKTVKASDVASLVMAKTRGVNKPSVE | |
| | GLSDPKKFNSNLATYLDLKSIVGDKVDDNRYQTDLENIIEWRSVFEDGEIFADKLTEVEWLTDEQRSA | |
| | LVKKRYKGWGRLSKKLLTGIVDENGQRIIDLMWNTDQNFKEIVDQPVFKEQIDQLNQKAITNDGMTLR | |
| | ERVESVLDDAYTSPQNKKAIWQVVRVVEDIVKAVGNAPKSISIEFARNEGNKGEITRSRRTQLQKLFE | |
| | DQAHELVKDTSLTEELEKAPDLSDRYYFYFTQGGKDMYTGDPINFDEISTKYDIDHILPQSFVKDNSL | |
| | DNRVLTSRKENNKKSDQVPAKLYAAKMKPYWNQLLKQGLITQRKFENLTKDVDQNIKYRSLGFVKRQL | |
| | VETRQVIKLTANILGSMYQEAGTEIIETRAGLTKQLREEFDLPKVREVNDYHHAVDAYLTTFAGQYLN | |
| | RRYPKLRSFFVYGEYMKFKHGSDLKLRNFNFFHELMEGDKSQGKWDQQTGELITTRDEVAKSFDRLL | |
| | NMKYMLVSKEVHDRSDQLYGATIVTAKESGKLTSPIEIKKNRLVDLYGAYTNGTSAFMTIIKFTGNKP | |
| | KYKVIGIPTTSAASLKRAGKPGSESYNQELHRIIKSNPKVKKGFEIVVPHVSYGQLIVDGDCKFTLAS | |
| | PTVQHPATQLVLSKKSLETISSGYKILKDKPAIANERLIRVFDEWGQMNRYFTIFDQRSNRQKVADA | |
| | RDKFLSLPTESKYEGAKKVQVGKTEVITNLLMGLHANATQGDLKVLGLATFGFFQSTTGLSLSEDTMI | |
| | VYQSPTGLFERRICLKDI | |
| SaCas9 | MDKKYSIGLDIGTNSVGWAVITDEYKVPSKKFKVLGNTDRHSIKKNLIGALLFDSGETAEATRLKRTA | 14 |
| Staphylococcus | RRRYTRRKNRICYLQEIFSNEMAKVDDSFFHRLEESFLVEEDKKHERHPIFGNIVDEVAYHEKYPTIY | |
| aureus wild | HLRKKLVDSTDKADLRLIYLALAHMIKFRGHFLIEGDLNPDNSDVDKLFIQLVQTYNQLFEENPINAS | |
| type | GVDAKAILSARLSKSRRLENLIAQLPGEKKNGLFGNLIALSLGLTPNFKSNFDLAEDAKLQLSKDTYD | |
| GenBank: | DDLDNLLAQIGDQYADLFLAAKNLSDAILLSDILRVNTEITKAPLSASMIKRYDEHHQDLTLLKALVR | |
| AYD60528.1 | QQLPEKYKEIFFDQSKNGYAGYIDGGASQEEFYKFIKPILEKMDGTEELLVKLNREDLLRKQRTFDNG | |
| | SIPHQIHLGELHAILRRQEDFYPFLKDNREKIEKILTFRIPYYVGPLARGNSRFAWMTRKSEETITPW | |
| | NFEEVVDKGASAQSFIERMTNFDKNLPNEKVLPKHSLLYEYFTVYNELTKVKYVTEGMRKPAFLSGEQ | |
| | KKAIVDLLFKTNRKVTVKQLKEDYFKKIECFDSVEISGVEDRFNASLGTYHDLLKIIKDKDFLDNEEN | |
| | EDILEDIVLTLTLFEDREMIEERLKTYAHLFDDKVMKQLKRRRYTGWGRLSRKLINGIRDKQSGKTIL | |
| | DFLKSDGFANRNFMQLIHDDSLTFKEDIQKAQVSGQGDSLHEHIANLAGSPAIKKGILQTVKVVDELV | |
| | KVMGRHKPENIVIEMARENQTTQKGQKNSRERMKRIEEGIKELGSQILKEHPVENTQLQNEKLYLYYL | |
| | QNGRDMYVDQELDINRLSDYDVDHIVPQSFLKDDSIDNKVLTRSDKNRGKSDNVPSEEVVKKMKNYWR | |
| | QLLNAKLITQRKFDNLTKAERGGLSELDKAGFIKRQLVETRQITKHVAQILDSRMNTKYDENDKLIRE | |
| | VKVITLKSKLVSDFRKDFQFYKVREINNYHHAHDAYLNAVVGTALIKKYPKLESEFVYGDYKVYDVRK | |
| | MIAKSEQEIGKATAKYFFYSNIMNFFKTEITLANGEIRKRPLIETNGETGEIVWDKGRDFATVRKVLS | |
| | MPQVNIVKKTEVQTGGFSKESILPKRNSDKLIARKKDWDPKKYGGFDSPTVAYSVLVVAKVEKGKSKK | |
| | LKSVKELLGITIMERSSFEKNPIDFLEAKGYKEVKKDLIIKLPKYSLFELENGRKRMLASAGELQKGN | |
| | ELALPSKYVNFLYLASHYEKLKGSPEDNEQKQLFVEQHKHYLDEIIEQISEFSKRVILADANLDKVLS | |
| | AYNKHRDKPIREQAENIIHLFTLTNLGAPAAFKYFDTTIDRKRYTSTKEVLDATLIHQSITGLYETRI | |
| | DLSQLGGD | |
| SaCas9 | MGKRNYILGLDIGITSVGYGIIDYETRDVIDAGVRLFKEANVENNEGRRSKRGARRLKRRRRHRIQRV | 15 |
| Staphylococcus | KKLLFDYNLLTDHSELSGINPYEARVKGLSQKLSEEEFSAALLHLAKRRGVHNVNEVEEDTGNELSTK | |
| aureus | EQISRNSKALEEKYVAELQLERLKKDGEVRGSINRFKTSDYVKEAKQLLKVQKAYHQLDQSFIDTYID | |
| | LLETRRTYYEGPGEGSPFGWKDIKEWYEMLMGHCTYFPEELRSVKYAYNADLYNALNDLNNLVITRDE | |
| | NEKLEYYEKFQIIENVFKQKKKPTLKQIAKEILVNEEDIKGYRVTSTGKPEFTNLKVYHDIKDITARK | |
| | EIIENAELLDQIAKILTIYQSSEDIQEELTNLNSELTQEEIEQISNLKGYTGTHNLSLKAINLILDEL | |
| | WHTNDNQIAIFNRLKLVPKKVDLSQQKEIPTTLVDDFILSPVVKRSFIQSIKVINAIIKKYGLPNDII | |
| | IELAREKNSKDAQKMINEMQKRNRQTNERIEEIIRTTGKENAKYLIEKIKLHDMQEGKCLYSLEAIPL | |
| | EDLLNNPFNYEVDHIIPRSVSFDNSFNNKVLVKQEENSKKGNRTPFQYLSSSDSKISYETFKKHILNL | |
| | AKGKGRISKTKKEYLLEERDINRFSVQKDFINRNLVDTRYATRGLMNLLRSYFRVNNLDVKVKSINGG | |
| | FTSFLRRKWKFKKERNKGYKHHAEDALIIANADFIFKEWKKLDKAKKVMENQMFEEKQAESMPEIETE | |
| | QEYKEIFITPHQIKHIKDFKDYKYSHRVDKKPNRKLINDTLYSTRKDDKGNTLIVNNLNGLYDKDNDK | |
| | LKKLINKSPEKLLMYHHDPQTYQKLKLIMEQYGDEKNPLYKYYEETGNYLTKYSKKDNGPVIKKIKYY | |
| | GNKLNAHLDITDDYPNSRNKVVKLSLKPYRFDVYLDNGVYKFVTVKNLDVIKKENYYEVNSKCYEEAK | |
| | KLKKISNQAEFIASFYKNDLIKINGELYRVIGVNNDLLNRIEVNMIDITYREYLENMNDKRPPHIIKT | |
| | IASKTQSIKKYSTDILGNLYEVKSKKHPQIIKK | |
| StCas9 | MLFNKCIIISINLDFSNKEKCMTKPYSIGLDIGTNSVGWAVITDNYKVPSKKMKVLGNTSKKYIKKNL | 16 |
| Streptococcus | LGVLLFDSGITAEGRRLKRTARRRYTRRRNRILYLQEIFSTEMATLDDAFFQRLDDSFLVPDDKRDSK | |
| thermophilus | YPIFGNLVEEKVYHDEFPTIYHLRKYLADSTKKADLRLVYLALAHMIKYRGHFLIEGEFNSKNNDIQK | |
| UniProtKB/ | NFQDFLDTYNAIFESDLSLENSKQLEEIVKDKISKLEKKDRILKLFPGEKNSGIFSEFLKLIVGNQAD | |
| Swiss-Prot: | FRKCFNLDEKASLHFSKESYDEDLETLLGYIGDDYSDVFLKAKKLYDAILLSGFLTVTDNETEAPLSS | |
| G3ECR1.2 | AMIKRYNEHKEDLALLKEYIRNISLKTYNEVFKDDTKNGYAGYIDGKTNQEDFYVYLKNLLAEFEGAD | |
| Wild type | YFLEKIDREDFLRKQRTFDNGSIPYQIHLQEMRAILDKQAKFYPFLAKNKERIEKILTFRIPYYVGPL | |
| | ARGNSDFAWSIRKRNEKITPWNFEDVIDKESSAEAFINRMTSFDLYLPEEKVLPKHSLLYETFNVYNE | |
| | LTKVRFIAESMRDYQFLDSKQKKDIVRLYFKDKRKVTDKDIIEYLHAIYGYDGIELKGIEKQFNSSLS | |
| | TYHDLLNIINDKEFLDDSSNEAIIEEIIHTLTIFEDREMIKQRLSKFENIFDKSVLKKLSRRHYTGWG | |
| | KLSAKLINGIRDEKSGNTILDYLIDDGISNRNFMQLIHDDALSFKKKIQKAQIIGDEDKGNIKEVVKS | |
| | LPGSPAIKKGILQSIKIVDELVKVMGGRKPESIVVEMARENQYTNQGKSNSQQRLKRLEKSLKELGSK | |
| | ILKENIPAKLSKIDNNALQNDRLYLYYLQNGKDMYTGDDLDIDRLSNYDIDHIIPQAFLKDNSIDNKV | |
| | LVSSASNRGKSDDFPSLEVVKKRKTFWYQLLKSKLISQRKFDNLTKAERGGLLPEDKAGFIQRQLVET | |
| | RQITKHVARLLDEKFNNKKDENNRAVRTVKIITLKSTLVSQFRKDFELYKVREINDFHHAHDAYLNAV | |
| | IASALLKKYPKLEPEFVYGDYPKYNSFRERKSATEKVYFYSNIMNIFKKSISLADGRVIERPLIEVNE | |
| | ETGESVWNKESDLATVRRVLSYPQVNVVKKVEEQNHGLDRGKPKGLFNANLSSKPKPNSNENLVGAKE | |
| | YLDPKKYGGYAGISNSFAVLVKGTIEKGAKKKITNVLEFQGISILDRINYRKDKLNFLLEKGYKDIEL | |
| | IIELPKYSLFELSDGSRRMLASILSTNNKRGEIHKGNQIFLSQKFVKLLYHAKRISNTINENHRKYVE | |
| | NHKKEFEELFYYILEFNENYVGAKKNGKLLNSAFQSWQNHSIDELCSSFIGPTGSERKGLFELTSRGS | |
| | AADFEFLGVKIPRYRDYTPSSLLKDATLIHQSVTGLYETRIDLAKLGEG | |
| LcCas9 | MKIKNYNLALTPSTSAVGHVEVDDDLNILEPVHHQKAIGVAKFGEGETAEARRLARSARRTTKRRANR | 17 |
| Lactobacillus | INHYFNEIMKPEIDKVDPLMFDRIKQAGLSPLDERKEFRTVIFDRPNIASYYHNQFPTIWHLQKYLMI | |
| crispatus | TDEKADIRLIYWALHSLLKHRGHFFNTTPMSQFKPGKLNLKDDMLALDDYNDLEGLSFAVANSPEIEK | |
| NCBI Reference | VIKDRSMHKKEKIAELKKLIVNDVPDKDLAKRNNKIITQIVNAIMGNSFHLNFIFDMDLDKLTSKAWS | |
| Sequence: | FKLDDPELDTKFDAISGSMTDNQIGIFETLQKIYSAISLLDILNGSSNVVDAKNALYDKHKRDLNLYF | |
| WP_133478044.1 | KFLNTLPDEIAKTLKAGYTLYIGNRKKDLLAARKLLKVNVAKNFSQDDFYKLINKELKSIDKQGLQTR | |
| Wild type | FSEKVGELVAQNNFLPVQRSSDNVFIPYQLNAITFNKILENQGKYYDFLVKPNPAKKDRKNAPYELSQ | |
| | LMQFTIPYYVGPLVTPEEQVKSGIPKTSRFAWMVRKDNGAITPWNFYDKVDIEATADKFIKRSIAKDS | |
| | YLLSELVLPKHSLLYEKYEVFNELSNVSLDGKKLSGGVKQILFNEVFKKTNKVNTSRILKALAKHNIP | |
| | GSKITGLSNPEEFTSSLQTYNAWKKYFPNQIDNFAYQQDLEKMIEWSTVFEDHKILAKKLDEIEWLDD | |
| | DQKKFVANTRLRGWGRLSKRLLTGLKDNYGKSIMQRLETTKANFQQIVYKPEFREQIDKISQAAAKNQ | |
| | SLEDILANSYTSPSNRKAIRKTMSVVDEYIKLNHGKEPDKIFLMFQRSEQEKGKQTEARSKQLNRILS | |
| | QLKADKSANKLFSKQLADEFSNAIKKSKYKLNDKQYFYFQQLGRDALTGEVIDYDELYKYTVLHIIPR | |
| | SKLTDDSQNNKVLTKYKIVDGSVALKFGNSYSDALGMPIKAFWTELNRLKLIPKGKLLNLTTDFSTLN | |
| | KYQRDGYIARQLVETQQIVKLLATIMQSRFKHTKIIEVRNSQVANIRYQFDYFRIKNLNEYYRGFDAY | |
| | LAAVVGTYLYKVYPKARRLFVYGQYLKPKKTNQENQDMHLDSEKKSQGFNFLWNLLYGKQDQIFVNGT | |
| | DVIAFNRKDLITKMNTVYNYKSQKISLAIDYHNGAMFKATLFPRNDRDTAKTRKLIPKKKDYDTDIYG | |
| | GYTSNVDGYMLLAEIIKRDGNKQYGFYGVPSRLVSELDTLKKTRYTEYEEKLKEIIKPELGVDLKKIK | |
| | KIKILKNKVPFNQVIIDKGSKFFITSTSYRWNYRQLILSAESQQTLMDLVVDPDFSNHKARKDARKNA | |
| | DERLIKVYEEILYQVKNYMPMFVELHRCYEKLVDAQKTFKSLKISDKAMVLNQILILLHSNATSPVLE | |
| | KLGYHTRFTLGKKHNLISENAVLVTQSITGLKENHVSIKQML | |
| PdCas9 | MTNEKYSIGLDIGTSSIGFAVVNDNNRVIRVKGKNAIGVRLFDEGKAAADRRSFRTTRRSFRTTRRRL | 18 |
| Pedicoccus | SRRRWRLKLLREIFDAYITPVDEAFFIRLKESNLSPKDSKKQYSGDILFNDRSDKDFYEKYPTIYHLR | |
| damnosus | NALMTEHRKFDVREIYLAIHHIMKFRGHFLNATPANNFKVGRLNLEEKFEELNDIYQRVFPDESIEFR | |
| NCBI Reference | TDNLEQIKEVLLDNKRSRADRQRTLVSDIYQSSEDKDIEKRNKAVATEILKASLGNKAKLNVITNVEV | |
| Sequence: | DKEAAKEWSITFDSESIDDDLAKIEGQMTDDGHEIIEVLRSLYSGITLSAIVPENHTLSQSMVAKYDL | |
| WP_062913273.1 | HKDHLKLFKKLINGMTDTKKAKNLRAAYDGYIDGVKGKVLPQEDFYKQVQVNLDDSAEANEIQTYIDQ | |
| Wild type | DIFMPKQRTKANGSIPHQLQQQELDQIIENQKAYYPWLAELNPNPDKKRQQLAKYKLDELVTFRVPYY | |
| | VGPMITAKDQKNQSGAEFAWMIRKEPGNITPWNFDQKVDRMATANQFIKRMTTTDTYLLGEDVLPAQS | |
| | LLYQKFEVLNELNKIRIDHKPISIEQKQQIFNDLFKQFKNVTIKHLQDYLVSQGQYSKRPLIEGLADE | |
| | KRFNSSLSTYSDLCGIFGAKLVEENDRQEDLEKIIEWSTIFEDKKIYRAKLNDLTWLTDDQKEKLATK | |
| | RYQGWGRLSRKLLVGLKNSEHRNIMDILWITNENFMQIQAEPDFAKLVTDANKGMLEKTDSQDVINDL | |
| | YTSPQNKKAIRQILLVVHDIQNAMHGQAPAKIHVEFARGEERNPRRSVQRQRQVEAAYEKVSNELVSA | |
| | KVRQEFKEAINNKRDFKDRLFLYFMQGGIDIYTGKQLNIDQLSSYQIDHILPQAFVKDDSLTNRVLTN | |
| | ENQVKADSVPIDIFGKKMLSVWGRMKDQGLISKGKYRNLTMNPENISAHTENGFINRQLVETRQVIKL | |
| | AVNILADEYGDSTQIISVKADLSHQMREDFELLKNRDVNDYHHAFDAYLAAFIGNYLLKRYPKLESYF | |
| | VYGDFKKFTQKETKMRRFNFIYDLKHCDQVVNKETGEILWTKDEDIKYIRHLFAYKKILVSHEVREKR | |
| | GALYNQTIYKAKDDKGSGQESKKLIRIKDDKETKIYGGYSGKSLAYMTIVQITKKNKVSYRVIGIPTL | |
| | ALARLNKLENDSTENNGELYKIIKPQFTHYKVDKKNGEIIETTDDFKIVVSKVRFQQLIDDAGQFFML | |
| | ASDTYKNNAQQLVISNNALKAINNTNITDCPRDDLERLDNLRLDSAFDEIVKKMDKYFSAYDANNFRE | |
| | KIRNSNLIFYQLPVEDQWENNKITELGKRTVLTRILQGLHANATTTDMSIFKIKTPFGQLRQRSGISL | |
| | SENAQLIYQSPTGLFERRVQLNKIK | |
| FnCas9 | MKKQKFSDYYLGFDIGTNSVGWCVTDLDYNVLRFNKKDMWGSRLFEEAKTAAERRVQRNSRRRLKRRK | 19 |
| Fusobaterium | WRLNLLEEIFSNEILKIDSNFFRRLKESSLWLEDKSSKEKFTLFNDDNYKDYDFYKQYPTIFHLRNEL | |
| nucleatum | IKNPEKKDIRLVYLAIHSIFKSRGHFLFEGQNLKEIKNFETLYNNLIAFLEDNGINKIIDKNNIEKLE | |
| NCBI Reference | KIVCDSKKGLKDKEKEFKEIFNSDKQLVAIFKLSVGSSVSLNDLFDTDEYKKGEVEKEKISFREQIYE | |
| Sequence: | DDKPIYYSILGEKIELLDIAKTFYDFMVLNNILADSQYISEAKVKLYEEHKKDLKNLKYIIRKYNKGN | |
| WP_060798984.1 | YDKLFKDKNENNYSAYIGLNKEKSKKEVIEKSRLKIDDLIKNIKGYLPKVEEIEEKDKAIFNKILNKI | |
| | ELKTILPKQRISDNGTLPYQIHEAELEKILENQSKYYDFLNYEENGIITKDKLLMTFKFRIPYYVGPL | |
| | NSYHKDKGGNSWIVRKEEGKILPWNFEQKVDIEKSAEEFIKRMTNKCTYLNGEDVIPKDTFLYSEYVI | |
| | LNELNKVQVNDEFLNEENKRKIIDELFKENKKVSEKKFKEYLLVKQIVDGTIELKGVKDSFNSNYISY | |
| | IRFKDIFGEKLNLDIYKEISEKSILWKCLYGDDKKIFEKKIKNEYGDILTKDEIKKINTFKFNNWGRL | |
| | SEKLLTGIEFINLETGECYSSVMDALRRTNYNLMELLSSKFTLQESINNENKEMNEASYRDLIEESYV | |
| | SPSLKRAIFQTLKIYEEIRKITGRVPKKVFIEMARGGDESMKNKKIPARQEQLKKLYDSCGNDIANFS | |
| | IDIKEMKNSLISYDNNSLRQKKLYLYYLQFGKCMYTGREIDLDRLLQNNDTYDIDHIYPRSKVIKDDS | |
| | FDNLVLVLKNENAEKSNEYPVKKEIQEKMKSFWRFLKEKNFISDEKYKRLTGKDDFELRGFMARQLVN | |
| | VRQTTKEVGKILQQIEPEIKIVYSKAEIASSFREMFDFIKVRELNDTHHAKDAYLNIVAGNVYNTKFT | |
| | EKPYRYLQEIKENYDVKKIYNYDIKNAWDKENSLEIVKKNMEKNTVNITRFIKEKKGQLFDLNPIKKG | |
| | ETSNEIISIKPKVYNGKDDKLNEKYGYYKSLNPAYFLYVEHKEKNKRIKSFERVNLVDVNNIKDEKSL | |
| | VKYLIENKKLVEPRVIKKVYKRQVILINDYPYSIVTLDSNKLMDFENLKPLFLENKYEKILKNVIKFL | |
| | EDNQGKSEENYKFIYLKKKDRYEKNETLESVKDRYNLEFNEMYDKFLEKLDSKDYKNYMNNKKYQELL | |
| | DVKEKFIKLNLFDKAFTLKSFLDLFNRKTMADFSKVGLTKYLGKIQKISSNVLSKNELYLLEESVTGL | |
| | FVKKIKL | |
| EcCas9 | MNKYYLGLDMGSASVGWAVTDENYHLVRRKGKDLWGVRTFDVAQTAKERRITRGNRRRQDRRKQRIQI | 20 |
| Enterococcus | LQELLGEEVLKTDPGFFHRMKESRYVVEDKRTLDGKQVELPYALFVDKDYTDKEYYKQFPTINHLIVY | |
| cecorum | LMTTSDTPDIRLVYLALHYYMKNRGNFLHSGDINNVKDINDILEQLDNVLETFLDGWNLKLKSYVEDI | |
| NCBI Reference | KNIYNRDLGRGERKKAFVNTLGAKTKAEKAFCSLISGGSTNLAELFDDSSLKEIETPKIEFASSSLED | |
| Sequence: | KIDGIQEALEDRFAVIEAAKRLYDWKTLTDILGDSSSLAEARVNSYQMHHEQLLELKSLVKEYLDRKV | |
| WP_047338501.1 | FQEVFVSLNVANNYPAYIGHTKINGKKKELEVKRTKRNDFYSYVKKQVIEPIKKKVSDEAVLTKLSEI | |
| Wild type | ESLIEVDKYLPLQVNSDNGVIPYQVKLNELTRIFDNLENRIPVLRENRDKIIKTFKFRIPYYVGSLNG | |
| | VVKNGKCTNWMVRKEEGKIYPWNFEDKVDLEASAEQFIRRMTNKCTYLVNEDVLPKYSLLYSKYLVLS | |
| | ELNNLRIDGRPLDVKIKQDIYENVFKKNRKVTLKKIKKYLLKEGIITDDDELSGLADDVKSSLTAYRD | |
| | FKEKLGHLDLSEAQMENIILNITLFGDDKKLLKKRLAALYPFIDDKSLNRIATLNYRDWGRLSERFLS | |
| | GITSVDQETGELRTIIQCMYETQANLMQLLAEPYHFVEAIEKENPKVDLESISYRIVNDLYVSPAVKR | |
| | QIWQTLLVIKDIKQVMKHDPERIFIEMAREKQESKKTKSRKQVLSEVYKKAKEYEHLFEKLNSLTEEQ | |
| | LRSKKIYLYFTQLGKCMYSGEPIDFENLVSANSNYDIDHIYPQSKTIDDSFNNIVLVKKSLNAYKSNH | |
| | YPIDKNIRDNEKVKTLWNTLVSKGLITKEKYERLIRSTPFSDEELAGFIARQLVETRQSTKAVAEILS | |
| | NWFPESEIVYSKAKNVSNFRQDFEILKVRELNDCHHAHDAYLNIVVGNAYHTKFTNSPYRFIKNKANQ | |
| | EYNLRKLLQKVNKIESNGVVAWVGQSENNPGTIATVKKVIRRNTVLISRMVKEVDGQLFDLTLMKKGK | |
| | GQVPIKSSDERLTDISKYGGYNKATGAYFTFVKSKKRGKVVRSFEYVPLHLSKQFENNNELLKEYIEK | |
| | DRGLTDVEILIPKVLINSLFRYNGSLVRITGRGDTRLLLVHEQPLYVSNSFVQQLKSVSSYKLKKSEN | |
| | DNAKLTKTATEKLSNIDELYDGLLRKLDLPIYSYWFSSIKEYLVESRTKYIKLSIEEKALVIFEILHL | |
| | FQSDAQVPNLKILGLSTKPSRIRIQKNLKDTDKMSIIHQSPSGIFEHEIELTSL | |
| AhCas 9 | MQNGFLGITVSSEQVGWAVTNPKYELERASRKDLWGVRLFDKAETAEDRRMFRTNRRLNQRKKNRIHY | 21 |
| Anaerostipes | LRDIFHEEVNQKDPNFFQQLDESNFCEDDRTVEFNFDTNLYKNQFPTVYHLRKYLMETKDKPDIRLVY | |
| hadrus | LAFSKFMKNRGHFLYKGNLGEVMDFENSMKGFCESLEKFNIDFPTLSDEQVKEVRDILCDHKIAKTVK | |
| NCBI Reference | KKNIITITKVKSKTAKAWIGLFCGCSVPVKVLFQDIDEEIVTDPEKISFEDASYDDYIANIEKGVGIY | |
| Sequence: | YEAIVSAKMLFDWSILNEILGDHQLLSDAMIAEYNKHHDDLKRLQKIIKGTGSRELYQDIFINDVSGN | |
| WP_044924278.1 | YVCYVGHAKTMSSADQKQFYTFLKNRLKNVNGISSEDAEWIDTEIKNGTLLPKQTKRDNSVIPHQLQL | |
| Wild type | REFELILDNMQEMYPFLKENREKLLKIFNFVIPYYVGPLKGVVRKGESTNWMVPKKDGVIHPWNFDEM | |
| | VDKEASAECFISRMTGNCSYLFNEKVLPKNSLLYETFEVLNELNPLKINGEPISVELKQRIYEQLFLT | |
| | GKKVTKKSLTKYLIKNGYDKDIELSGIDNEFHSNLKSHIDFEDYDNLSDEEVEQIILRITVFEDKQLL | |
| | KDYLNREFVKLSEDERKQICSLSYKGWGNLSEMLLNGITVTDSNGVEVSVMDMLWNTNLNLMQILSKK | |
| | YGYKAEIEHYNKEHEKTIYNREDLMDYLNIPPAQRRKVNQLITIVKSLKKTYGVPNKIFFKISREHQD | |
| | DPKRTSSRKEQLKYLYKSLKSEDEKHLMKELDELNDHELSNDKVYLYFLQKGRCIYSGKKLNLSRLRK | |
| | SNYQNDIDYIYPLSAVNDRSMNNKVLTGIQENRADKYTYFPVDSEIQKKMKGFWMELVLQGFMTKEKY | |
| | FRLSRENDFSKSELVSFIEREISDNQQSGRMIASVLQYYFPESKIVFVKEKLISSFKRDFHLISSYGH | |
| | NHLQAAKDAYITIVVGNVYHTKFTMDPAIYFKNHKRKDYDLNRLFLENISRDGQIAWESGPYGSIQTV | |
| | RKEYAQNHIAVTKRVVEVKGGLFKQMPLKKGHGEYPLKTNDPRFGNIAQYGGYTNVTGSYFVLVESME | |
| | KGKKRISLEYVPVYLHERLEDDPGHKLLKEYLVDHRKLNHPKILLAKVRKNSLLKIDGFYYRLNGRSG | |
| | NALILTNAVELIMDDWQTKTANKISGYMKRRAIDKKARVYQNEFHIQELEQLYDFYLDKLKNGVYKNR | |
| | KNNQAELIHNEKEQFMELKTEDQCVLLTEIKKLFVCSPMQADLTLIGGSKHTGMIAMSSNVTKADFAV | |
| | IAEDPLGLRNKVIYSHKGEK | |
| KvCas9 | MSQNNNKIYNIGLDIGDASVGWAVVDEHYNLLKRHGKHMWGSRLFTQANTAVERRSSRSTRRRYNKRR | 22 |
| Kandleria | ERIRLLREIMEDMVLDVDPTFFIRLANVSFLDQEDKKDYLKENYHSNYNLFIDKDFNDKTYYDKYPTI | |
| vitulina | YHLRKHLCESKEKEDPRLIYLALHHIVKYRGNFLYEGQKFSMDVSNIEDKMIDVLRQFNEINLFEYVE | |
| NCBI Reference | DRKKIDEVLNVLKEPLSKKHKAEKAFALFDTTKDNKAAYKELCAALAGNKFNVTKMLKEAELHDEDEK | |
| Sequence: | DISFKFSDATFDDAFVEKQPLLGDCVEFIDLLHDIYSWVELQNILGSAHTSEPSISAAMIQRYEDHKN | |
| WP_031589969.1 | DLKLLKDVIRKYLPKKYFEVFRDEKSKKNNYCNYINHPSKTPVDEFYKYIKKLIEKIDDPDVKTILNK | |
| Wild type | IELESFMLKQNSRTNGAVPYQMQLDELNKILENQSVYYSDLKDNEDKIRSILTFRIPYYFGPLNITKD | |
| | RQFDWIIKKEGKENERILPWNANEIVDVDKTADEFIKRMRNFCTYFPDEPVMAKNSLTVSKYEVLNEI | |
| | NKLRINDHLIKRDMKDKMLHTLFMDHKSISANAMKKWLVKNQYFSNTDDIKIEGFQKENACSTSLTPW | |
| | IDFTKIFGKINESNYDFIEKIIYDVTVFEDKKILRRRLKKEYDLDEEKIKKILKLKYSGWSRLSKKLL | |
| | SGIKTKYKDSTRTPETVLEVMERTNMNLMQVINDEKLGFKKTIDDANSTSVSGKFSYAEVQELAGSPA | |
| | IKRGIWQALLIVDEIKKIMKHEPAHVYIEFARNEDEKERKDSFVNQMLKLYKDYDFEDETEKEANKHL | |
| | KGEDAKSKIRSERLKLYYTQMGKCMYTGKSLDIDRLDTYQVDHIVPQSLLKDDSIDNKVLVLSSENQR | |
| | KLDDLVIPSSIRNKMYGFWEKLFNNKIISPKKFYSLIKTEFNEKDQERFINRQIVETRQITKHVAQII | |
| | DNHYENTKVVTVRADLSHQFRERYHIYKNRDINDFHHAHDAYIATILGTYIGHRFESLDAKYIYGEYK | |
| | RIFRNQKNKGKEMKKNNDGFILNSMRNIYADKDTGEIVWDPNYIDRIKKCFYYKDCFVTKKLEENNGT | |
| | FFNVTVLPNDTNSDKDNTLATVPVNKYRSNVNKYGGFSGVNSFIVAIKGKKKKGKKVIEVNKLTGIPL | |
| | MYKNADEEIKINYLKQAEDLEEVQIGKEILKNQLIEKDGGLYYIVAPTEIINAKQLILNESQTKLVCE | |
| | IYKAMKYKNYDNLDSEKIIDLYRLLINKMELYYPEYRKQLVKKFEDRYEQLKVISIEEKCNIIKQILA | |
| | TLHCNSSIGKIMYSDFKISTTIGRLNGRTISLDDISFIAESPTGMYSKKYKL | |
| EfCas9 | MRLFEEGHTAEDRRLKRTARRRISRRRNRLRYLQAFFEEAMTDLDENFFARLQESFLVPEDKKWHRHP | 23 |
| Enterococcus | IFAKLEDEVAYHETYPTIYHLRKKLADSSEQADLRLIYLALAHIVKYRGHFLIEGKLSTENTSVKDQF | |
| faecalis | QQFMVIYNQTFVNGESRLVSAPLPESVLIEEELTEKASRTKKSEKVLQQFPQEKANGLFGQFLKLMVG | |
| NCBI Reference | NKADFKKVFGLEEEAKITYASESYEEDLEGILAKVGDEYSDVFLAAKNVYDAVELSTILADSDKKSHA | |
| Sequence: | KLSSSMIVRFTEHQEDLKKFKRFIRENCPDEYDNLFKNEQKDGYAGYIAHAGKVSQLKFYQYVKKIIQ | |
| WP_016631044.1 | DIAGAEYFLEKIAQENFLRKQRTFDNGVIPHQIHLAELQAIIHRQAAYYPFLKENQEKIEQLVTFRIP | |
| Wild type | YYVGPLSKGDASTFAWLKRQSEEPIRPWNLQETVDLDQSATAFIERMTNFDTYLPSEKVLPKHSLLYE | |
| | KFMVFNELTKISYTDDRGIKANFSGKEKEKIFDYLFKTRRKVKKKDIIQFYRNEYNTEIVTLSGLEED | |
| | QFNASFSTYQDLLKCGLTRAELDHPDNAEKLEDIIKILTIFEDRQRIRTQLSTFKGQFSAEVLKKLER | |
| | KHYTGWGRLSKKLINGIYDKESGKTILDYLVKDDGVSKHYNRNFMQLINDSQLSFKNAIQKAQSSEHE | |
| | ETLSETVNELAGSPAIKKGIYQSLKIVDELVAIMGYAPKRIVVEMARENQTTSTGKRRSIQRLKIVEK | |
| | AMAEIGSNLLKEQPTTNEQLRDTRLFLYYMQNGKDMYTGDELSLHRLSHYDIDHIIPQSFMKDDSLDN | |
| | LVLVGSTENRGKSDDVPSKEVVKDMKAYWEKLYAAGLISQRKFQRLTKGEQGGLTLEDKAHFIQRQLV | |
| | ETRQITKNVAGILDQRYNAKSKEKKVQIITLKASLTSQFRSIFGLYKVREVNDYHHGQDAYLNCVVAT | |
| | TLLKVYPNLAPEFVYGEYPKFQTFKENKATAKAIIYTNLLRFFTEDEPRFTKDGEILWSNSYLKTIKK | |
| | ELNYHQMNIVKKVEVQKGGFSKESIKPKGPSNKLIPVKNGLDPQKYGGFDSPVVAYTVLFTHEKGKKP | |
| | LIKQEILGITIMEKTRFEQNPILFLEEKGFLRPRVLMKLPKYTLYEFPEGRRRLLASAKEAQKGNQMV | |
| | LPEHLLTLLYHAKQCLLPNQSESLAYVEQHQPEFQEILERVVDFAEVHTLAKSKVQQIVKLFEANQTA | |
| | DVKEIAASFIQLMQFNAMGAPSTFKFFQKDIERARYTSIKEIFDATIIYQSPTGLYETRRKVVD | |
| Staphylococcus | KRNYILGLDIGITSVGYGIIDYETRDVIDAGVRLFKEANVENNEGRRSKRGARRLKRRRRHRIQRVKK | 24 |
| aureus Cas9 | LLFDYNLLTDHSELSGINPYEARVKGLSQKLSEEEFSAALLHLAKRRGVHNVNEVEEDTGNELSTKEQ | |
| | ISRNSKALEEKYVAELQLERLKKDGEVRGSINRFKTSDYVKEAKQLLKVQKAYHQLDQSFIDTYIDLL | |
| | ETRRTYYEGPGEGSPFGWKDIKEWYEMLMGHCTYFPEELRSVKYAYNADLYNALNDLNNLVITRDENE | |
| | KLEYYEKFQIIENVFKQKKKPTLKQIAKEILVNEEDIKGYRVTSTGKPEFTNLKVYHDIKDITARKEI | |
| | IENAELLDQIAKILTIYQSSEDIQEELTNLNSELTQEEIEQISNLKGYTGTHNLSLKAINLILDELWH | |
| | TNDNQIAIFNRLKLVPKKVDLSQQKEIPTTLVDDFILSPVVKRSFIQSIKVINAIIKKYGLPNDIIIE | |
| | LAREKNSKDAQKMINEMQKRNRQTNERIEEIIRTTGKENAKYLIEKIKLHDMQEGKCLYSLEAIPLED | |
| | LLNNPFNYEVDHIIPRSVSFDNSFNNKVLVKQEENSKKGNRTPFQYLSSSDSKISYETFKKHILNLAK | |
| | GKGRISKTKKEYLLEERDINRFSVQKDFINRNLVDTRYATRGLMNLLRSYFRVNNLDVKVKSINGGFT | |
| | SFLRRKWKFKKERNKGYKHHAEDALIIANADFIFKEWKKLDKAKKVMENQMFEEKQAESMPEIETEQE | |
| | YKEIFITPHQIKHIKDFKDYKYSHRVDKKPNRELINDTLYSTRKDDKGNTLIVNNLNGLYDKDNDKLK | |
| | KLINKSPEKLLMYHHDPQTYQKLKLIMEQYGDEKNPLYKYYEETGNYLTKYSKKDNGPVIKKIKYYGN | |
| | KLNAHLDITDDYPNSRNKVVKLSLKPYRFDVYLDNGVYKFVTVKNLDVIKKENYYEVNSKCYEEAKKL | |
| | KKISNQAEFIASFYNNDLIKINGELYRVIGVNNDLLNRIEVNMIDITYREYLENMNDKRPPRIIKTIA | |
| | SKTQSIKKYSTDILGNLYEVKSKKHPQIIKKG | |
| Geobacillus | MKYKIGLDIGITSIGWAVINLDIPRIEDLGVRIFDRAENPKTGESLALPRRLARSARRRLRRRKHRLE | 25 |
| thermo- | RIRRLFVREGILTKEELNKLFEKKHEIDVWQLRVEALDRKLNNDELARILLHLAKRRGFRSNRKSERT | |
| denitrificans | NKENSTMLKHIEENQSILSSYRTVAEMVVKDPKFSLHKRNKEDNYTNTVARDDLEREIKLIFAKQREY | |
| Cas9 | GNIVCTEAFEHEYISIWASQRPFASKDDIEKKVGFCTFEPKEKRAPKATYTFQSFTVWEHINKLRLVS | |
| | PGGIRALTDDERRLIYKQAFHKNKITFHDVRTLLNLPDDTRFKGLLYDRNTTLKENEKVRFLELGAYH | |
| | KIRKAIDSVYGKGAAKSFRPIDFDTFGYALTMFKDDTDIRSYLRNEYEQNGKRMENLADKVYDEELIE | |
| | ELLNLSFSKFGHLSLKALRNILPYMEQGEVYSTACERAGYTFTGPKKKQKTVLLPNIPPIANPVVMRA | |
| | LTQARKVVNAIIKKYGSPVSIHIELARELSQSFDERRKMQKEQEGNRKKNETAIRQLVEYGLTLNPTG | |
| | LDIVKFKLWSEQNGKCAYSLQPIEIERLLEPGYTEVDHVIPYSRSLDDSYTNKVLVLTKENREKGNRT | |
| | PAEYLGLGSERWQQFETFVLTNKQFSKKKRDRLLRLHYDENEENEFKNRNLNDTRYISRFLANFIREH | |
| | LKFADSDDKQKVYTVNGRITAHLRSRWNFNKNREESNLHHAVDAAIVACTTPSDIARVTAFYQRREQN | |
| | KELSKKTDPQFPQPWPHFADELQARLSKNPKESIKALNLGNYDNEKLESLQPVFVSRMPKRSITGAAH | |
| | QETLRRYIGIDERSGKIQTVVKKKLSEIQLDKTGHFPMYGKESDPRTYEAIRQRLLEHNNDPKKAFQE | |
| | PLYKPKKNGELGPIIRTIKIIDTTNQVIPLNDGKTVAYNSNIVRVDVFEKDGKYYCVPIYTIDMMKGI | |
| | LPNKAIEPNKPYSEWKEMTEDYTFRFSLYPNDLIRIEFPREKTIKTAVGEEIKIKDLFAYYQTIDSSN | |
| | GGLSLVSHDNNFSLRSIGSRTLKRFEKYQVDVLGNIYKVRGEKRVGVASSSHSKAGETIRPL | |
| ScCas9 | MEKKYSIGLDIGTNSVGWAVITDDYKVPSKKFKVLGNTNRKSIKKNLMGALLFDSGETAEATRLKRTA | 26 |
| S. canis | RRRYTRRKNRIRYLQEIFANEMAKLDDSFFQRLEESFLVEEDKKNERHPIFGNLADEVAYHRNYPTIY | |
| 1375 AA | HLRKKLADSPEKADLRLIYLALAHIIKFRGHFLIEGKLNAENSDVAKLFYQLIQTYNQLFEESPLDEI | |
| 159.2 kDa | EVDAKGILSARLSKSKRLEKLIAVFPNEKKNGLFGNIIALALGLTPNFKSNFDLTEDAKLQLSKDTYD | |
| | DDLDELLGQIGDQYADLFSAAKNLSDAILLSDILRSNSEVTKAPLSASMVKRYDEHHQDLALLKTLVR | |
| | QQFPEKYAEIFKDDTKNGYAGYVGIGIKHRKRTTKLATQEEFYKFIKPILEKMDGAEELLAKLNRDDL | |
| | LRKQRTFDNGSIPHQIHLKELHAILRRQEEFYPFLKENREKIEKILTFRIPYYVGPLARGNSRFAWLT | |
| | RKSEEAITPWNFEEVVDKGASAQSFIERMTNFDEQLPNKKVLPKHSLLYEYFTVYNELTKVKYVTERM | |
| | RKPEFLSGEQKKAIVDLLFKTNRKVTVKQLKEDYFKKIECFDSVEIIGVEDRFNASLGTYHDLLKIIK | |
| | DKDFLDNEENEDILEDIVLTLTLFEDREMIEERLKTYAHLFDDKVMKQLKRRHYTGWGRLSRKMINGI | |
| | RDKQSGKTILDFLKSDGFSNRNFMQLIHDDSLTFKEEIEKAQVSGQGDSLHEQIADLAGSPAIKKGIL | |
| | QTVKIVDELVKVMGHKPENIVIEMARENQTTTKGLQQSRERKKRIEEGIKELESQILKENPVENTQLQ | |
| | NEKLYLYYLQNGRDMYVDQELDINRLSDYDVDHIVPQSFIKDDSIDNKVLTRSVENRGKSDNVPSEEV | |
| | VKKMKNYWRQLLNAKLITQRKFDNLTKAERGGLSEADKAGFIKRQLVETRQITKHVARILDSRMNTKR | |
| | DKNDKPIREVKVITLKSKLVSDFRKDFQLYKVRDINNYHHAHDAYLNAVVGTALIKKYPKLESEFVYG | |
| | DYKVYDVRKMIAKSEQEIGKATAKRFFYSNIMNFFKTEVKLANGEIRKRPLIETNGETGEVVWNKEKD | |
| | FATVRKVLAMPQVNIVKKTEVQTGGFSKESILSKRESAKLIPRKKGWDTRKYGGFGSPTVAYSILVVA | |
| | KVEKGKAKKLKSVKVLVGITIMEKGSYEKDPIGFLEAKGYKDIKKELIFKLPKYSLFELENGRRRMLA | |
| | SATELQKANELVLPQHLVRLLYYTQNISATTGSNNLGYIEQHREEFKEIFEKIIDFSEKYILKNKVNS | |
| | NLKSSFDEQFAVSDSILLSNSFVSLLKYTSFGASGGFTFLDLDVKQGRLRYQTVTEVLDATLIYQSIT | |
| | GLYETRTDLSQLGGD | |

Dead Cas9 variant:

| | | |
|---|---|---|
| dead Cas9 or | MDKKYSIGLXIGTNSVGWAVITDEYKVPSKKFKVLGNTDRHSIKKNLIGALLFDSGETAEATRLKRTA | 27 |
| dCas 9 | RRRYTRRKNRICYLQEIFSNEMAKVDDSFFHRLEESFLVEEDKKHERHPIFGNIVDEVAYHEKYPTIY | |
| Streptococcus | HLRKKLVDSTDKADLRLIYLALAHMIKFRGHFLIEGDLNPDNSDVDKLFIQLVQTYNQLFEENPINAS | |
| pyogenes | GVDAKAILSARLSKSRRLENLIAQLPGEKKNGLFGNLIALSLGLTPNFKSNFDLAEDAKLQLSKDTYD | |
| Q99ZW2 Cas9 | DDLDNLLAQIGDQYADLFLAAKNLSDAILLSDILRVNTEITKAPLSASMIKRYDEHHQDLTLLKALVR | |
| with D10X and | QQLPEKYKEIFFDQSKNGYAGYIDGGASQEEFYKFIKPILEKMDGTEELLVKLNREDLLRKQRTFDNG | |
| H810X | SIPHQIHLGELHAILRRQEDFYPFLKDNREKIEKILTFRIPYYVGPLARGNSRFAWMTRKSEETITPW | |
| Where ″X″ is | NFEEVVDKGASAQSFIERMTNFDKNLPNEKVLPKHSLLYEYFTVYNELTKVKYVTEGMRKPAFLSGEQ | |
| any amino acid | KKAIVDLLFKTNRKVTVKQLKEDYFKKIECFDSVEISGVEDRFNASLGTYHDLLKIIKDKDFLDNEEN | |
| | EDILEDIVLTLTLFEDREMIEERLKTYAHLFDDKVMKQLKRRRYTGWGRLSRKLINGIRDKQSGKTIL | |
| | DFLKSDGFANRNFMQLIHDDSLTFKEDIQKAQVSGQGDSLHEHIANLAGSPAIKKGILQTVKVVDELV | |
| | KVMGRHKPENIVIEMARENQTTQKGQKNSRERMKRIEEGIKELGSQILKEHPVENTQLQNEKLYLYYL | |
| | QNGRDMYVDQELDINRLSDYDVDXIVPQSFLKDDSIDNKVLTRSDKNRGKSDNVPSEEVVKKMKNYWR | |
| | QLLNAKLITQRKFDNLTKAERGGLSELDKAGFIKRQLVETRQITKHVAQILDSRMNTKYDENDKLIRE | |
| | VKVITLKSKLVSDFRKDFQFYKVREINNYHHAHDAYLNAVVGTALIKKYPKLESEFVYGDYKVYDVRK | |
| | MIAKSEQEIGKATAKYFFYSNIMNFFKTEITLANGEIRKRPLIETNGETGEIVWDKGRDFATVRKVLS | |
| | MPQVNIVKKTEVQTGGFSKESILPKRNSDKLIARKKDWDPKKYGGFDSPTVAYSVLVVAKVEKGKSKK | |
| | LKSVKELLGITIMERSSFEKNPIDFLEAKGYKEVKKDLIIKLPKYSLFELENGRKRMLASAGELQKGN | |
| | ELALPSKYVNFLYLASHYEKLKGSPEDNEQKQLFVEQHKHYLDEIIEQISEFSKRVILADANLDKVLS | |
| | AYNKHRDKPIREQAENIIHLFTLTNLGAPAAFKYFDTTIDRKRYTSTKEVLDATLIHQSITGLYETRI | |
| | DLSQLGGD | |
| dead Cas9 or | MDKKYSIGLAIGTNSVGWAVITDEYKVPSKKFKVLGNTDRHSIKKNLIGALLFDSGETAEATRLKRTA | 28 |
| dCas 9 | RRRYTRRKNRICYLQEIFSNEMAKVDDSFFHRLEESFLVEEDKKHERHPIFGNIVDEVAYHEKYPTIY | |
| Streptococcus | HLRKKLVDSTDKADLRLIYLALAHMIKFRGHFLIEGDLNPDNSDVDKLFIQLVQTYNQLFEENPINAS | |
| pyogenes | GVDAKAILSARLSKSRRLENLIAQLPGEKKNGLFGNLIALSLGLTPNFKSNFDLAEDAKLQLSKDTYD | |
| Q99ZW2 Cas9 | DDLDNLLAQIGDQYADLFLAAKNLSDAILLSDILRVNTEITKAPLSASMIKRYDEHHQDLTLLKALVR | |
| with D10A and | QQLPEKYKEIFFDQSKNGYAGYIDGGASQEEFYKFIKPILEKMDGTEELLVKLNREDLLRKQRTFDNG | |
| H810A | SIPHQIHLGELHAILRRQEDFYPFLKDNREKIEKILTFRIPYYVGPLARGNSRFAWMTRKSEETITPW | |
| | NFEEVVDKGASAQSFIERMTNFDKNLPNEKVLPKHSLLYEYFTVYNELTKVKYVTEGMRKPAFLSGEQ | |
| | KKAIVDLLFKTNRKVTVKQLKEDYFKKIECFDSVEISGVEDRFNASLGTYHDLLKIIKDKDFLDNEEN | |
| | EDILEDIVLTLTLFEDREMIEERLKTYAHLFDDKVMKQLKRRRYTGWGRLSRKLINGIRDKQSGKTIL | |
| | DFLKSDGFANRNFMQLIHDDSLTFKEDIQKAQVSGQGDSLHEHIANLAGSPAIKKGILQTVKVVDELV | |
| | KVMGRHKPENIVIEMARENQTTQKGQKNSRERMKRIEEGIKELGSQILKEHPVENTQLQNEKLYLYYL | |
| | QNGRDMYVDQELDINRLSDYDVDAIVPQSFLKDDSIDNKVLTRSDKNRGKSDNVPSEEVVKKMKNYWR | |
| | QLLNAKLITQRKFDNLTKAERGGLSELDKAGFIKRQLVETRQITKHVAQILDSRMNTKYDENDKLIRE | |
| | VKVITLKSKLVSDFRKDFQFYKVREINNYHHAHDAYLNAVVGTALIKKYPKLESEFVYGDYKVYDVRK | |
| | MIAKSEQEIGKATAKYFFYSNIMNFFKTEITLANGEIRKRPLIETNGETGEIVWDKGRDFATVRKVLS | |
| | MPQVNIVKKTEVQTGGFSKESILPKRNSDKLIARKKDWDPKKYGGFDSPTVAYSVLVVAKVEKGKSKK | |
| | LKSVKELLGITIMERSSFEKNPIDFLEAKGYKEVKKDLIIKLPKYSLFELENGRKRMLASAGELQKGN | |
| | ELALPSKYVNFLYLASHYEKLKGSPEDNEQKQLFVEQHKHYLDEIIEQISEFSKRVILADANLDKVLS | |
| | AYNKHRDKPIREQAENIIHLFTLTNLGAPAAFKYFDTTIDRKRYTSTKEVLDATLIHQSITGLYETRI | |
| | DLSQLGGD | |

Cas9 nickase variant

| | | |
|---|---|---|
| Cas9 nickase | MDKKYSIGLXIGTNSVGWAVITDEYKVPSKKFKVLGNTDRHSIKKNLIGALLFDSGETAEATRLKRTA | 29 |
| Streptococcus | RRRYTRRKNRICYLQEIFSNEMAKVDDSFFHRLEESFLVEEDKKHERHPIFGNIVDEVAYHEKYPTIY | |
| pyogenes | HLRKKLVDSTDKADLRLIYLALAHMIKFRGHFLIEGDLNPDNSDVDKLFIQLVQTYNQLFEENPINAS | |
| Q99ZW2 Cas9 | GVDAKAILSARLSKSRRLENLIAQLPGEKKNGLFGNLIALSLGLTPNFKSNFDLAEDAKLQLSKDTYD | |
| with D10X, | DDLDNLLAQIGDQYADLFLAAKNLSDAILLSDILRVNTEITKAPLSASMIKRYDEHHQDLTLLKALVR | |
| wherein X is | QQLPEKYKEIFFDQSKNGYAGYIDGGASQEEFYKFIKPILEKMDGTEELLVKLNREDLLRKQRTFDNG | |
| any alternate | SIPHQIHLGELHAILRRQEDFYPFLKDNREKIEKILTFRIPYYVGPLARGNSRFAWMTRKSEETITPW | |
| amino acid | NFEEVVDKGASAQSFIERMTNFDKNLPNEKVLPKHSLLYEYFTVYNELTKVKYVTEGMRKPAFLSGEQ | |
| | KKAIVDLLFKTNRKVTVKQLKEDYFKKIECFDSVEISGVEDRFNASLGTYHDLLKIIKDKDFLDNEEN | |
| | EDILEDIVLTLTLFEDREMIEERLKTYAHLFDDKVMKQLKRRRYTGWGRLSRKLINGIRDKQSGKTIL | |
| | DFLKSDGFANRNFMQLIHDDSLTFKEDIQKAQVSGQGDSLHEHIANLAGSPAIKKGILQTVKVVDELV | |
| | KVMGRHKPENIVIEMARENQTTQKGQKNSRERMKRIEEGIKELGSQILKEHPVENTQLQNEKLYLYYL | |
| | QNGRDMYVDQELDINRLSDYDVDHIVPQSFLKDDSIDNKVLTRSDKNRGKSDNVPSEEVVKKMKNYWR | |
| | QLLNAKLITQRKFDNLTKAERGGLSELDKAGFIKRQLVETRQITKHVAQILDSRMNTKYDENDKLIRE | |
| | VKVITLKSKLVSDFRKDFQFYKVREINNYHHAHDAYLNAVVGTALIKKYPKLESEFVYGDYKVYDVRK | |
| | MIAKSEQEIGKATAKYFFYSNIMNFFKTEITLANGEIRKRPLIETNGETGEIVWDKGRDFATVRKVLS | |
| | MPQVNIVKKTEVQTGGFSKESILPKRNSDKLIARKKDWDPKKYGGFDSPTVAYSVLVVAKVEKGKSKK | |
| | LKSVKELLGITIMERSSFEKNPIDFLEAKGYKEVKKDLIIKLPKYSLFELENGRKRMLASAGELQKGN | |
| | ELALPSKYVNFLYLASHYEKLKGSPEDNEQKQLFVEQHKHYLDEIIEQISEFSKRVILADANLDKVLS | |
| | AYNKHRDKPIREQAENIIHLFTLTNLGAPAAFKYFDTTIDRKRYTSTKEVLDATLIHQSITGLYETRI | |
| | DLSQLGGD | |
| Cas9 nickase | MDKKYSIGLDIGTNSVGWAVITDEYKVPSKKFKVLGNTDRHSIKKNLIGALLFDSGETAEATRLKRTA | 30 |
| Streptococcus | RRRYTRRKNRICYLQEIFSNEMAKVDDSFFHRLEESFLVEEDKKHERHPIFGNIVDEVAYHEKYPTIY | |
| pyogenes | HLRKKLVDSTDKADLRLIYLALAHMIKFRGHFLIEGDLNPDNSDVDKLFIQLVQTYNQLFEENPINAS | |
| Q99ZW2 Cas9 | GVDAKAILSARLSKSRRLENLIAQLPGEKKNGLFGNLIALSLGLTPNFKSNFDLAEDAKLQLSKDTYD | |
| with E762X, | DDLDNLLAQIGDQYADLFLAAKNLSDAILLSDILRVNTEITKAPLSASMIKRYDEHHQDLTLLKALVR | |
| wherein X is | QQLPEKYKEIFFDQSKNGYAGYIDGGASQEEFYKFIKPILEKMDGTEELLVKLNREDLLRKQRTFDNG | |
| any alternate | SIPHQIHLGELHAILRRQEDFYPFLKDNREKIEKILTFRIPYYVGPLARGNSRFAWMTRKSEETITPW | |
| amino acid | NFEEVVDKGASAQSFIERMTNFDKNLPNEKVLPKHSLLYEYFTVYNELTKVKYVTEGMRKPAFLSGEQ | |
| | KKAIVDLLFKTNRKVTVKQLKEDYFKKIECFDSVEISGVEDRFNASLGTYHDLLKIIKDKDFLDNEEN | |
| | EDILEDIVLTLTLFEDREMIEERLKTYAHLFDDKVMKQLKRRRYTGWGRLSRKLINGIRDKQSGKTIL | |
| | DFLKSDGFANRNFMQLIHDDSLTFKEDIQKAQVSGQGDSLHEHIANLAGSPAIKKGILQTVKVVDELV | |
| | KVMGRHKPENIVIXMARENQTTQKGQKNSRERMKRIEEGIKELGSQILKEHPVENTQLQNEKLYLYYL | |
| | QNGRDMYVDQELDINRLSDYDVDHIVPQSFLKDDSIDNKVLTRSDKNRGKSDNVPSEEVVKKMKNYWR | |
| | QLLNAKLITQRKFDNLTKAERGGLSELDKAGFIKRQLVETRQITKHVAQILDSRMNTKYDENDKLIRE | |
| | VKVITLKSKLVSDFRKDFQFYKVREINNYHHAHDAYLNAVVGTALIKKYPKLESEFVYGDYKVYDVRK | |
| | MIAKSEQEIGKATAKYFFYSNIMNFFKTEITLANGEIRKRPLIETNGETGEIVWDKGRDFATVRKVLS | |
| | MPQVNIVKKTEVQTGGFSKESILPKRNSDKLIARKKDWDPKKYGGFDSPTVAYSVLVVAKVEKGKSKK | |
| | LKSVKELLGITIMERSSFEKNPIDFLEAKGYKEVKKDLIIKLPKYSLFELENGRKRMLASAGELQKGN | |
| | ELALPSKYVNFLYLASHYEKLKGSPEDNEQKQLFVEQHKHYLDEIIEQISEFSKRVILADANLDKVLS | |
| | AYNKHRDKPIREQAENIIHLFTLTNLGAPAAFKYFDTTIDRKRYTSTKEVLDATLIHQSITGLYETRI | |
| | DLSQLGGD | |
| Cas9 nickase | MDKKYSIGLDIGTNSVGWAVITDEYKVPSKKFKVLGNTDRHSIKKNLIGALLFDSGETAEATRLKRTA | 31 |
| Streptococcus | RRRYTRRKNRICYLQEIFSNEMAKVDDSFFHRLEESFLVEEDKKHERHPIFGNIVDEVAYHEKYPTIY | |
| pyogenes | HLRKKLVDSTDKADLRLIYLALAHMIKFRGHFLIEGDLNPDNSDVDKLFIQLVQTYNQLFEENPINAS | |
| Q99ZW2 Cas9 | GVDAKAILSARLSKSRRLENLIAQLPGEKKNGLFGNLIALSLGLTPNFKSNFDLAEDAKLQLSKDTYD | |
| with H983X, | DDLDNLLAQIGDQYADLFLAAKNLSDAILLSDILRVNTEITKAPLSASMIKRYDEHHQDLTLLKALVR | |
| wherein X is | QQLPEKYKEIFFDQSKNGYAGYIDGGASQEEFYKFIKPILEKMDGTEELLVKLNREDLLRKQRTFDNG | |
| any alternate | SIPHQIHLGELHAILRRQEDFYPFLKDNREKIEKILTFRIPYYVGPLARGNSRFAWMTRKSEETITPW | |
| amino acid | NFEEVVDKGASAQSFIERMTNFDKNLPNEKVLPKHSLLYEYFTVYNELTKVKYVTEGMRKPAFLSGEQ | |
| | KKAIVDLLFKTNRKVTVKQLKEDYFKKIECFDSVEISGVEDRFNASLGTYHDLLKIIKDKDFLDNEEN | |
| | EDILEDIVLTLTLFEDREMIEERLKTYAHLFDDKVMKQLKRRRYTGWGRLSRKLINGIRDKQSGKTIL | |
| | DFLKSDGFANRNFMQLIHDDSLTFKEDIQKAQVSGQGDSLHEHIANLAGSPAIKKGILQTVKVVDELV | |
| | KVMGRHKPENIVIEMARENQTTQKGQKNSRERMKRIEEGIKELGSQILKEHPVENTQLQNEKLYLYYL | |
| | QNGRDMYVDQELDINRLSDYDVDHIVPQSFLKDDSIDNKVLTRSDKNRGKSDNVPSEEVVKKMKNYWR | |
| | QLLNAKLITQRKFDNLTKAERGGLSELDKAGFIKRQLVETRQITKHVAQILDSRMNTKYDENDKLIRE | |
| | VKVITLKSKLVSDFRKDFQFYKVREINNYHXAHDAYLNAVVGTALIKKYPKLESEFVYGDYKVYDVRK | |
| | MIAKSEQEIGKATAKYFFYSNIMNFFKTEITLANGEIRKRPLIETNGETGEIVWDKGRDFATVRKVLS | |
| | MPQVNIVKKTEVQTGGFSKESILPKRNSDKLIARKKDWDPKKYGGFDSPTVAYSVLVVAKVEKGKSKK | |
| | LKSVKELLGITIMERSSFEKNPIDFLEAKGYKEVKKDLIIKLPKYSLFELENGRKRMLASAGELQKGN | |
| | ELALPSKYVNFLYLASHYEKLKGSPEDNEQKQLFVEQHKHYLDEIIEQISEFSKRVILADANLDKVLS | |
| | AYNKHRDKPIREQAENIIHLFTLTNLGAPAAFKYFDTTIDRKRYTSTKEVLDATLIHQSITGLYETRI | |
| | DLSQLGGD | |
| Cas9 nickase | MDKKYSIGLDIGTNSVGWAVITDEYKVPSKKFKVLGNTDRHSIKKNLIGALLFDSGETAEATRLKRTA | 32 |
| Streptococcus | RRRYTRRKNRICYLQEIFSNEMAKVDDSFFHRLEESFLVEEDKKHERHPIFGNIVDEVAYHEKYPTIY | |
| pyogenes | HLRKKLVDSTDKADLRLIYLALAHMIKFRGHFLIEGDLNPDNSDVDKLFIQLVQTYNQLFEENPINAS | |
| Q99ZW2 Cas9 | GVDAKAILSARLSKSRRLENLIAQLPGEKKNGLFGNLIALSLGLTPNFKSNFDLAEDAKLQLSKDTYD | |
| with D986X, | DDLDNLLAQIGDQYADLFLAAKNLSDAILLSDILRVNTEITKAPLSASMIKRYDEHHQDLTLLKALVR | |
| wherein X is | QQLPEKYKEIFFDQSKNGYAGYIDGGASQEEFYKFIKPILEKMDGTEELLVKLNREDLLRKQRTFDNG | |
| any alternate | SIPHQIHLGELHAILRRQEDFYPFLKDNREKIEKILTFRIPYYVGPLARGNSRFAWMTRKSEETITPW | |
| amino acid | NFEEVVDKGASAQSFIERMTNFDKNLPNEKVLPKHSLLYEYFTVYNELTKVKYVTEGMRKPAFLSGEQ | |
| | KKAIVDLLFKTNRKVTVKQLKEDYFKKIECFDSVEISGVEDRFNASLGTYHDLLKIIKDKDFLDNEEN | |
| | EDILEDIVLTLTLFEDREMIEERLKTYAHLFDDKVMKQLKRRRYTGWGRLSRKLINGIRDKQSGKTIL | |
| | DFLKSDGFANRNFMQLIHDDSLTFKEDIQKAQVSGQGDSLHEHIANLAGSPAIKKGILQTVKVVDELV | |
| | KVMGRHKPENIVIEMARENQTTQKGQKNSRERMKRIEEGIKELGSQILKEHPVENTQLQNEKLYLYYL | |
| | QNGRDMYVDQELDINRLSDYDVDHIVPQSFLKDDSIDNKVLTRSDKNRGKSDNVPSEEVVKKMKNYWR | |
| | QLLNAKLITQRKFDNLTKAERGGLSELDKAGFIKRQLVETRQITKHVAQILDSRMNTKYDENDKLIRE | |
| | VKVITLKSKLVSDFRKDFQFYKVREINNYHHAHXAYLNAVVGTALIKKYPKLESEFVYGDYKVYDVRK | |
| | MIAKSEQEIGKATAKYFFYSNIMNFFKTEITLANGEIRKRPLIETNGETGEIVWDKGRDFATVRKVLS | |
| | MPQVNIVKKTEVQTGGFSKESILPKRNSDKLIARKKDWDPKKYGGFDSPTVAYSVLVVAKVEKGKSKK | |
| | LKSVKELLGITIMERSSFEKNPIDFLEAKGYKEVKKDLIIKLPKYSLFELENGRKRMLASAGELQKGN | |
| | ELALPSKYVNFLYLASHYEKLKGSPEDNEQKQLFVEQHKHYLDEIIEQISEFSKRVILADANLDKVLS | |
| | AYNKHRDKPIREQAENIIHLFTLTNLGAPAAFKYFDTTIDRKRYTSTKEVLDATLIHQSITGLYETRI | |
| | DLSQLGGD | |
| Cas9 nickase | MDKKYSIGLAIGTNSVGWAVITDEYKVPSKKFKVLGNTDRHSIKKNLIGALLFDSGETAEATRLKRTA | 33 |
| Streptococcus | RRRYTRRKNRICYLQEIFSNEMAKVDDSFFHRLEESFLVEEDKKHERHPIFGNIVDEVAYHEKYPTIY | |
| pyogenes | HLRKKLVDSTDKADLRLIYLALAHMIKFRGHFLIEGDLNPDNSDVDKLFIQLVQTYNQLFEENPINAS | |
| Q99ZW2 Cas9 | GVDAKAILSARLSKSRRLENLIAQLPGEKKNGLFGNLIALSLGLTPNFKSNFDLAEDAKLQLSKDTYD | |
| with D10A | DDLDNLLAQIGDQYADLFLAAKNLSDAILLSDILRVNTEITKAPLSASMIKRYDEHHQDLTLLKALVR | |
| | QQLPEKYKEIFFDQSKNGYAGYIDGGASQEEFYKFIKPILEKMDGTEELLVKLNREDLLRKQRTFDNG | |
| | SIPHQIHLGELHAILRRQEDFYPFLKDNREKIEKILTFRIPYYVGPLARGNSRFAWMTRKSEETITPW | |
| | NFEEVVDKGASAQSFIERMTNFDKNLPNEKVLPKHSLLYEYFTVYNELTKVKYVTEGMRKPAFLSGEQ | |
| | KKAIVDLLFKTNRKVTVKQLKEDYFKKIECFDSVEISGVEDRFNASLGTYHDLLKIIKDKDFLDNEEN | |
| | EDILEDIVLTLTLFEDREMIEERLKTYAHLFDDKVMKQLKRRRYTGWGRLSRKLINGIRDKQSGKTIL | |
| | DFLKSDGFANRNFMQLIHDDSLTFKEDIQKAQVSGQGDSLHEHIANLAGSPAIKKGILQTVKVVDELV | |
| | KVMGRHKPENIVIEMARENQTTQKGQKNSRERMKRIEEGIKELGSQILKEHPVENTQLQNEKLYLYYL | |
| | QNGRDMYVDQELDINRLSDYDVDHIVPQSFLKDDSIDNKVLTRSDKNRGKSDNVPSEEVVKKMKNYWR | |
| | QLLNAKLITQRKFDNLTKAERGGLSELDKAGFIKRQLVETRQITKHVAQILDSRMNTKYDENDKLIRE | |
| | VKVITLKSKLVSDFRKDFQFYKVREINNYHHAHDAYLNAVVGTALIKKYPKLESEFVYGDYKVYDVRK | |
| | MIAKSEQEIGKATAKYFFYSNIMNFFKTEITLANGEIRKRPLIETNGETGEIVWDKGRDFATVRKVLS | |
| | MPQVNIVKKTEVQTGGFSKESILPKRNSDKLIARKKDWDPKKYGGFDSPTVAYSVLVVAKVEKGKSKK | |
| | LKSVKELLGITIMERSSFEKNPIDFLEAKGYKEVKKDLIIKLPKYSLFELENGRKRMLASAGELQKGN | |
| | ELALPSKYVNFLYLASHYEKLKGSPEDNEQKQLFVEQHKHYLDEIIEQISEFSKRVILADANLDKVLS | |
| | AYNKHRDKPIREQAENIIHLFTLTNLGAPAAFKYFDTTIDRKRYTSTKEVLDATLIHQSITGLYETRI | |
| | DLSQLGGD | |
| Cas9 nickase | MDKKYSIGLDIGTNSVGWAVITDEYKVPSKKFKVLGNTDRHSIKKNLIGALLFDSGETAEATRLKRTA | 34 |
| Streptococcus | RRRYTRRKNRICYLQEIFSNEMAKVDDSFFHRLEESFLVEEDKKHERHPIFGNIVDEVAYHEKYPTIY | |
| pyogenes | HLRKKLVDSTDKADLRLIYLALAHMIKFRGHFLIEGDLNPDNSDVDKLFIQLVQTYNQLFEENPINAS | |
| Q99ZW2 Cas9 | GVDAKAILSARLSKSRRLENLIAQLPGEKKNGLFGNLIALSLGLTPNFKSNFDLAEDAKLQLSKDTYD | |
| with E762A | DDLDNLLAQIGDQYADLFLAAKNLSDAILLSDILRVNTEITKAPLSASMIKRYDEHHQDLTLLKALVR | |
| | QQLPEKYKEIFFDQSKNGYAGYIDGGASQEEFYKFIKPILEKMDGTEELLVKLNREDLLRKQRTFDNG | |
| | SIPHQIHLGELHAILRRQEDFYPFLKDNREKIEKILTFRIPYYVGPLARGNSRFAWMTRKSEETITPW | |
| | NFEEVVDKGASAQSFIERMTNFDKNLPNEKVLPKHSLLYEYFTVYNELTKVKYVTEGMRKPAFLSGEQ | |
| | KKAIVDLLFKTNRKVTVKQLKEDYFKKIECFDSVEISGVEDRFNASLGTYHDLLKIIKDKDFLDNEEN | |
| | EDILEDIVLTLTLFEDREMIEERLKTYAHLFDDKVMKQLKRRRYTGWGRLSRKLINGIRDKQSGKTIL | |
| | DFLKSDGFANRNFMQLIHDDSLTFKEDIQKAQVSGQGDSLHEHIANLAGSPAIKKGILQTVKVVDELV | |
| | KVMGRHKPENIVIAMARENQTTQKGQKNSRERMKRIEEGIKELGSQILKEHPVENTQLQNEKLYLYYL | |
| | QNGRDMYVDQELDINRLSDYDVDHIVPQSFLKDDSIDNKVLTRSDKNRGKSDNVPSEEVVKKMKNYWR | |
| | QLLNAKLITQRKFDNLTKAERGGLSELDKAGFIKRQLVETRQITKHVAQILDSRMNTKYDENDKLIRE | |
| | VKVITLKSKLVSDFRKDFQFYKVREINNYHHAHDAYLNAVVGTALIKKYPKLESEFVYGDYKVYDVRK | |
| | MIAKSEQEIGKATAKYFFYSNIMNFFKTEITLANGEIRKRPLIETNGETGEIVWDKGRDFATVRKVLS | |
| | MPQVNIVKKTEVQTGGFSKESILPKRNSDKLIARKKDWDPKKYGGFDSPTVAYSVLVVAKVEKGKSKK | |
| | LKSVKELLGITIMERSSFEKNPIDFLEAKGYKEVKKDLIIKLPKYSLFELENGRKRMLASAGELQKGN | |
| | ELALPSKYVNFLYLASHYEKLKGSPEDNEQKQLFVEQHKHYLDEIIEQISEFSKRVILADANLDKVLS | |
| | AYNKHRDKPIREQAENIIHLFTLTNLGAPAAFKYFDTTIDRKRYTSTKEVLDATLIHQSITGLYETRI | |
| | DLSQLGGD | |
| Cas9 nickase | MDKKYSIGLDIGTNSVGWAVITDEYKVPSKKFKVLGNTDRHSIKKNLIGALLFDSGETAEATRLKRTA | 35 |
| Streptococcus | RRRYTRRKNRICYLQEIFSNEMAKVDDSFFHRLEESFLVEEDKKHERHPIFGNIVDEVAYHEKYPTIY | |
| pyogenes | HLRKKLVDSTDKADLRLIYLALAHMIKFRGHFLIEGDLNPDNSDVDKLFIQLVQTYNQLFEENPINAS | |
| Q99ZW2 Cas9 | GVDAKAILSARLSKSRRLENLIAQLPGEKKNGLFGNLIALSLGLTPNFKSNFDLAEDAKLQLSKDTYD | |
| with H983A | DDLDNLLAQIGDQYADLFLAAKNLSDAILLSDILRVNTEITKAPLSASMIKRYDEHHQDLTLLKALVR | |
| | QQLPEKYKEIFFDQSKNGYAGYIDGGASQEEFYKFIKPILEKMDGTEELLVKLNREDLLRKQRTFDNG | |
| | SIPHQIHLGELHAILRRQEDFYPFLKDNREKIEKILTFRIPYYVGPLARGNSRFAWMTRKSEETITPW | |
| | NFEEVVDKGASAQSFIERMTNFDKNLPNEKVLPKHSLLYEYFTVYNELTKVKYVTEGMRKPAFLSGEQ | |
| | KKAIVDLLFKTNRKVTVKQLKEDYFKKIECFDSVEISGVEDRFNASLGTYHDLLKIIKDKDFLDNEEN | |
| | EDILEDIVLTLTLFEDREMIEERLKTYAHLFDDKVMKQLKRRRYTGWGRLSRKLINGIRDKQSGKTIL | |
| | DFLKSDGFANRNFMQLIHDDSLTFKEDIQKAQVSGQGDSLHEHIANLAGSPAIKKGILQTVKVVDELV | |
| | KVMGRHKPENIVIEMARENQTTQKGQKNSRERMKRIEEGIKELGSQILKEHPVENTQLQNEKLYLYYL | |
| | QNGRDMYVDQELDINRLSDYDVDHIVPQSFLKDDSIDNKVLTRSDKNRGKSDNVPSEEVVKKMKNYWR | |
| | QLLNAKLITQRKFDNLTKAERGGLSELDKAGFIKRQLVETRQITKHVAQILDSRMNTKYDENDKLIRE | |
| | VKVITLKSKLVSDFRKDFQFYKVREINNYHAAHDAYLNAVVGTALIKKYPKLESEFVYGDYKVYDVRK | |
| | MIAKSEQEIGKATAKYFFYSNIMNFFKTEITLANGEIRKRPLIETNGETGEIVWDKGRDFATVRKVLS | |
| | MPQVNIVKKTEVQTGGFSKESILPKRNSDKLIARKKDWDPKKYGGFDSPTVAYSVLVVAKVEKGKSKK | |
| | LKSVKELLGITIMERSSFEKNPIDFLEAKGYKEVKKDLIIKLPKYSLFELENGRKRMLASAGELQKGN | |
| | ELALPSKYVNFLYLASHYEKLKGSPEDNEQKQLFVEQHKHYLDEIIEQISEFSKRVILADANLDKVLS | |
| | AYNKHRDKPIREQAENIIHLFTLTNLGAPAAFKYFDTTIDRKRYTSTKEVLDATLIHQSITGLYETRI | |
| | DLSQLGGD | |
| Cas9 nickase | MDKKYSIGLDIGTNSVGWAVITDEYKVPSKKFKVLGNTDRHSIKKNLIGALLFDSGETAEATRLKRTA | 36 |
| Streptococcus | RRRYTRRKNRICYLQEIFSNEMAKVDDSFFHRLEESFLVEEDKKHERHPIFGNIVDEVAYHEKYPTIY | |
| pyogenes | HLRKKLVDSTDKADLRLIYLALAHMIKFRGHFLIEGDLNPDNSDVDKLFIQLVQTYNQLFEENPINAS | |
| Q99ZW2 Cas9 | GVDAKAILSARLSKSRRLENLIAQLPGEKKNGLFGNLIALSLGLTPNFKSNFDLAEDAKLQLSKDTYD | |
| with D986A | DDLDNLLAQIGDQYADLFLAAKNLSDAILLSDILRVNTEITKAPLSASMIKRYDEHHQDLTLLKALVR | |
| | QQLPEKYKEIFFDQSKNGYAGYIDGGASQEEFYKFIKPILEKMDGTEELLVKLNREDLLRKQRTFDNG | |
| | SIPHQIHLGELHAILRRQEDFYPFLKDNREKIEKILTFRIPYYVGPLARGNSRFAWMTRKSEETITPW | |
| | NFEEVVDKGASAQSFIERMTNFDKNLPNEKVLPKHSLLYEYFTVYNELTKVKYVTEGMRKPAFLSGEQ | |
| | KKAIVDLLFKTNRKVTVKQLKEDYFKKIECFDSVEISGVEDRFNASLGTYHDLLKIIKDKDFLDNEEN | |
| | EDILEDIVLTLTLFEDREMIEERLKTYAHLFDDKVMKQLKRRRYTGWGRLSRKLINGIRDKQSGKTIL | |
| | DFLKSDGFANRNFMQLIHDDSLTFKEDIQKAQVSGQGDSLHEHIANLAGSPAIKKGILQTVKVVDELV | |
| | KVMGRHKPENIVIEMARENQTTQKGQKNSRERMKRIEEGIKELGSQILKEHPVENTQLQNEKLYLYYL | |
| | QNGRDMYVDQELDINRLSDYDVDHIVPQSFLKDDSIDNKVLTRSDKNRGKSDNVPSEEVVKKMKNYWR | |
| | QLLNAKLITQRKFDNLTKAERGGLSELDKAGFIKRQLVETRQITKHVAQILDSRMNTKYDENDKLIRE | |
| | VKVITLKSKLVSDFRKDFQFYKVREINNYHHAHAAYLNAVVGTALIKKYPKLESEFVYGDYKVYDVRK | |
| | MIAKSEQEIGKATAKYFFYSNIMNFFKTEITLANGEIRKRPLIETNGETGEIVWDKGRDFATVRKVLS | |
| | MPQVNIVKKTEVQTGGFSKESILPKRNSDKLIARKKDWDPKKYGGFDSPTVAYSVLVVAKVEKGKSKK | |
| | LKSVKELLGITIMERSSFEKNPIDFLEAKGYKEVKKDLIIKLPKYSLFELENGRKRMLASAGELQKGN | |
| | ELALPSKYVNFLYLASHYEKLKGSPEDNEQKQLFVEQHKHYLDEIIEQISEFSKRVILADANLDKVLS | |
| | AYNKHRDKPIREQAENIIHLFTLTNLGAPAAFKYFDTTIDRKRYTSTKEVLDATLIHQSITGLYETRI | |
| | DLSQLGGD | |
| Cas9 nickase | MDKKYSIGLDIGTNSVGWAVITDEYKVPSKKFKVLGNTDRHSIKKNLIGALLFDSGETAEATRLKRTA | 37 |
| Streptococcus | RRRYTRRKNRICYLQEIFSNEMAKVDDSFFHRLEESFLVEEDKKHERHPIFGNIVDEVAYHEKYPTIY | |
| pyogenes | HLRKKLVDSTDKADLRLIYLALAHMIKFRGHFLIEGDLNPDNSDVDKLFIQLVQTYNQLFEENPINAS | |
| Q99ZW2 Cas9 | GVDAKAILSARLSKSRRLENLIAQLPGEKKNGLFGNLIALSLGLTPNFKSNFDLAEDAKLQLSKDTYD | |
| with H840X, | DDLDNLLAQIGDQYADLFLAAKNLSDAILLSDILRVNTEITKAPLSASMIKRYDEHHQDLTLLKALVR | |
| wherein X is | QQLPEKYKEIFFDQSKNGYAGYIDGGASQEEFYKFIKPILEKMDGTEELLVKLNREDLLRKQRTFDNG | |
| any alternate | SIPHQIHLGELHAILRRQEDFYPFLKDNREKIEKILTFRIPYYVGPLARGNSRFAWMTRKSEETITPW | |
| amino acid | NFEEVVDKGASAQSFIERMTNFDKNLPNEKVLPKHSLLYEYFTVYNELTKVKYVTEGMRKPAFLSGEQ | |
| | KKAIVDLLFKTNRKVTVKQLKEDYFKKIECFDSVEISGVEDRFNASLGTYHDLLKIIKDKDFLDNEEN | |
| | EDILEDIVLTLTLFEDREMIEERLKTYAHLFDDKVMKQLKRRRYTGWGRLSRKLINGIRDKQSGKTIL | |
| | DFLKSDGFANRNFMQLIHDDSLTFKEDIQKAQVSGQGDSLHEHIANLAGSPAIKKGILQTVKVVDELV | |
| | KVMGRHKPENIVIEMARENQTTQKGQKNSRERMKRIEEGIKELGSQILKEHPVENTQLQNEKLYLYYL | |
| | QNGRDMYVDQELDINRLSDYDVDXIVPQSFLKDDSIDNKVLTRSDKNRGKSDNVPSEEVVKKMKNYWR | |
| | QLLNAKLITQRKFDNLTKAERGGLSELDKAGFIKRQLVETRQITKHVAQILDSRMNTKYDENDKLIRE | |
| | VKVITLKSKLVSDFRKDFQFYKVREINNYHHAHDAYLNAVVGTALIKKYPKLESEFVYGDYKVYDVRK | |
| | MIAKSEQEIGKATAKYFFYSNIMNFFKTEITLANGEIRKRPLIETNGETGEIVWDKGRDFATVRKVLS | |
| | MPQVNIVKKTEVQTGGFSKESILPKRNSDKLIARKKDWDPKKYGGFDSPTVAYSVLVVAKVEKGKSKK | |
| | LKSVKELLGITIMERSSFEKNPIDFLEAKGYKEVKKDLIIKLPKYSLFELENGRKRMLASAGELQKGN | |
| | ELALPSKYVNFLYLASHYEKLKGSPEDNEQKQLFVEQHKHYLDEIIEQISEFSKRVILADANLDKVLS | |
| | AYNKHRDKPIREQAENIIHLFTLTNLGAPAAFKYFDTTIDRKRYTSTKEVLDATLIHQSITGLYETRI | |
| | DLSQLGGD | |
| Cas9 nickase | MDKKYSIGLDIGTNSVGWAVITDEYKVPSKKFKVLGNTDRHSIKKNLIGALLFDSGETAEATRLKRTA | 38 |
| Streptococcus | RRRYTRRKNRICYLQEIFSNEMAKVDDSFFHRLEESFLVEEDKKHERHPIFGNIVDEVAYHEKYPTIY | |
| pyogenes | HLRKKLVDSTDKADLRLIYLALAHMIKFRGHFLIEGDLNPDNSDVDKLFIQLVQTYNQLFEENPINAS | |
| Q99ZW2 Cas9 | GVDAKAILSARLSKSRRLENLIAQLPGEKKNGLFGNLIALSLGLTPNFKSNFDLAEDAKLQLSKDTYD | |
| with H840A, | DDLDNLLAQIGDQYADLFLAAKNLSDAILLSDILRVNTEITKAPLSASMIKRYDEHHQDLTLLKALVR | |
| wherein X is | QQLPEKYKEIFFDQSKNGYAGYIDGGASQEEFYKFIKPILEKMDGTEELLVKLNREDLLRKQRTFDNG | |
| any alternate | SIPHQIHLGELHAILRRQEDFYPFLKDNREKIEKILTFRIPYYVGPLARGNSRFAWMTRKSEETITPW | |
| amino acid | NFEEVVDKGASAQSFIERMTNFDKNLPNEKVLPKHSLLYEYFTVYNELTKVKYVTEGMRKPAFLSGEQ | |
| | KKAIVDLLFKTNRKVTVKQLKEDYFKKIECFDSVEISGVEDRFNASLGTYHDLLKIIKDKDFLDNEEN | |
| | EDILEDIVLTLTLFEDREMIEERLKTYAHLFDDKVMKQLKRRRYTGWGRLSRKLINGIRDKQSGKTIL | |
| | DFLKSDGFANRNFMQLIHDDSLTFKEDIQKAQVSGQGDSLHEHIANLAGSPAIKKGILQTVKVVDELV | |
| | KVMGRHKPENIVIEMARENQTTQKGQKNSRERMKRIEEGIKELGSQILKEHPVENTQLQNEKLYLYYL | |
| | QNGRDMYVDQELDINRLSDYDVDAIVPQSFLKDDSIDNKVLTRSDKNRGKSDNVPSEEVVKKMKNYWR | |
| | QLLNAKLITQRKFDNLTKAERGGLSELDKAGFIKRQLVETRQITKHVAQILDSRMNTKYDENDKLIRE | |
| | VKVITLKSKLVSDFRKDFQFYKVREINNYHHAHDAYLNAVVGTALIKKYPKLESEFVYGDYKVYDVRK | |
| | MIAKSEQEIGKATAKYFFYSNIMNFFKTEITLANGEIRKRPLIETNGETGEIVWDKGRDFATVRKVLS | |
| | MPQVNIVKKTEVQTGGFSKESILPKRNSDKLIARKKDWDPKKYGGFDSPTVAYSVLVVAKVEKGKSKK | |
| | LKSVKELLGITIMERSSFEKNPIDFLEAKGYKEVKKDLIIKLPKYSLFELENGRKRMLASAGELQKGN | |
| | ELALPSKYVNFLYLASHYEKLKGSPEDNEQKQLFVEQHKHYLDEIIEQISEFSKRVILADANLDKVLS | |
| | AYNKHRDKPIREQAENIIHLFTLTNLGAPAAFKYFDTTIDRKRYTSTKEVLDATLIHQSITGLYETRI | |
| | DLSQLGGD | |
| Cas9 nickase | MDKKYSIGLDIGTNSVGWAVITDEYKVPSKKFKVLGNTDRHSIKKNLIGALLFDSGETAEATRLKRTA | 39 |
| Streptococcus | RRRYTRRKNRICYLQEIFSNEMAKVDDSFFHRLEESFLVEEDKKHERHPIFGNIVDEVAYHEKYPTIY | |
| pyogenes | HLRKKLVDSTDKADLRLIYLALAHMIKFRGHFLIEGDLNPDNSDVDKLFIQLVQTYNQLFEENPINAS | |
| Q99ZW2 Cas9 | GVDAKAILSARLSKSRRLENLIAQLPGEKKNGLFGNLIALSLGLTPNFKSNFDLAEDAKLQLSKDTYD | |
| with R863X, | DDLDNLLAQIGDQYADLFLAAKNLSDAILLSDILRVNTEITKAPLSASMIKRYDEHHQDLTLLKALVR | |
| wherein X is | QQLPEKYKEIFFDQSKNGYAGYIDGGASQEEFYKFIKPILEKMDGTEELLVKLNREDLLRKQRTFDNG | |
| any alternate | SIPHQIHLGELHAILRRQEDFYPFLKDNREKIEKILTFRIPYYVGPLARGNSRFAWMTRKSEETITPW | |
| amino acid | NFEEVVDKGASAQSFIERMTNFDKNLPNEKVLPKHSLLYEYFTVYNELTKVKYVTEGMRKPAFLSGEQ | |
| | KKAIVDLLFKTNRKVTVKQLKEDYFKKIECFDSVEISGVEDRFNASLGTYHDLLKIIKDKDFLDNEEN | |
| | EDILEDIVLTLTLFEDREMIEERLKTYAHLFDDKVMKQLKRRRYTGWGRLSRKLINGIRDKQSGKTIL | |
| | DFLKSDGFANRNFMQLIHDDSLTFKEDIQKAQVSGQGDSLHEHIANLAGSPAIKKGILQTVKVVDELV | |
| | KVMGRHKPENIVIEMARENQTTQKGQKNSRERMKRIEEGIKELGSQILKEHPVENTQLQNEKLYLYYL | |
| | QNGRDMYVDQELDINRLSDYDVDHIVPQSFLKDDSIDNKVLTRSDKNXGKSDNVPSEEVVKKMKNYWR | |
| | QLLNAKLITQRKFDNLTKAERGGLSELDKAGFIKRQLVETRQITKHVAQILDSRMNTKYDENDKLIRE | |
| | VKVITLKSKLVSDFRKDFQFYKVREINNYHHAHDAYLNAVVGTALIKKYPKLESEFVYGDYKVYDVRK | |
| | MIAKSEQEIGKATAKYFFYSNIMNFFKTEITLANGEIRKRPLIETNGETGEIVWDKGRDFATVRKVLS | |
| | MPQVNIVKKTEVQTGGFSKESILPKRNSDKLIARKKDWDPKKYGGFDSPTVAYSVLVVAKVEKGKSKK | |
| | LKSVKELLGITIMERSSFEKNPIDFLEAKGYKEVKKDLIIKLPKYSLFELENGRKRMLASAGELQKGN | |
| | ELALPSKYVNFLYLASHYEKLKGSPEDNEQKQLFVEQHKHYLDEIIEQISEFSKRVILADANLDKVLS | |
| | AYNKHRDKPIREQAENIIHLFTLTNLGAPAAFKYFDTTIDRKRYTSTKEVLDATLIHQSITGLYETRI | |
| | DLSQLGGD | |
| Cas9 nickase | MDKKYSIGLDIGTNSVGWAVITDEYKVPSKKFKVLGNTDRHSIKKNLIGALLFDSGETAEATRLKRTA | 4 0 |
| Streptococcus | RRRYTRRKNRICYLQEIFSNEMAKVDDSFFHRLEESFLVEEDKKHERHPIFGNIVDEVAYHEKYPTIY | |
| pyogenes | HLRKKLVDSTDKADLRLIYLALAHMIKFRGHFLIEGDLNPDNSDVDKLFIQLVQTYNQLFEENPINAS | |
| Q99ZW2 Cas9 | GVDAKAILSARLSKSRRLENLIAQLPGEKKNGLFGNLIALSLGLTPNFKSNFDLAEDAKLQLSKDTYD | |
| with R863A, | DDLDNLLAQIGDQYADLFLAAKNLSDAILLSDILRVNTEITKAPLSASMIKRYDEHHQDLTLLKALVR | |
| wherein X is | QQLPEKYKEIFFDQSKNGYAGYIDGGASQEEFYKFIKPILEKMDGTEELLVKLNREDLLRKQRTFDNG | |
| any alternate | SIPHQIHLGELHAILRRQEDFYPFLKDNREKIEKILTFRIPYYVGPLARGNSRFAWMTRKSEETITPW | |
| amino acid | NFEEVVDKGASAQSFIERMTNFDKNLPNEKVLPKHSLLYEYFTVYNELTKVKYVTEGMRKPAFLSGEQ | |
| | KKAIVDLLFKTNRKVTVKQLKEDYFKKIECFDSVEISGVEDRFNASLGTYHDLLKIIKDKDFLDNEEN | |
| | EDILEDIVLTLTLFEDREMIEERLKTYAHLFDDKVMKQLKRRRYTGWGRLSRKLINGIRDKQSGKTIL | |
| | DFLKSDGFANRNFMQLIHDDSLTFKEDIQKAQVSGQGDSLHEHIANLAGSPAIKKGILQTVKVVDELV | |
| | KVMGRHKPENIVIEMARENQTTQKGQKNSRERMKRIEEGIKELGSQILKEHPVENTQLQNEKLYLYYL | |
| | QNGRDMYVDQELDINRLSDYDVDHIVPQSFLKDDSIDNKVLTRSDKNAGKSDNVPSEEVVKKMKNYWR | |
| | QLLNAKLITQRKFDNLTKAERGGLSELDKAGFIKRQLVETRQITKHVAQILDSRMNTKYDENDKLIRE | |
| | VKVITLKSKLVSDFRKDFQFYKVREINNYHHAHDAYLNAVVGTALIKKYPKLESEFVYGDYKVYDVRK | |
| | MIAKSEQEIGKATAKYFFYSNIMNFFKTEITLANGEIRKRPLIETNGETGEIVWDKGRDFATVRKVLS | |
| | MPQVNIVKKTEVQTGGFSKESILPKRNSDKLIARKKDWDPKKYGGFDSPTVAYSVLVVAKVEKGKSKK | |
| | LKSVKELLGITIMERSSFEKNPIDFLEAKGYKEVKKDLIIKLPKYSLFELENGRKRMLASAGELQKGN | |
| | ELALPSKYVNFLYLASHYEKLKGSPEDNEQKQLFVEQHKHYLDEIIEQISEFSKRVILADANLDKVLS | |
| | AYNKHRDKPIREQAENIIHLFTLTNLGAPAAFKYFDTTIDRKRYTSTKEVLDATLIHQSITGLYETRI | |
| | DLSQLGGD | |
| Cas9 nickase | DKKYSIGLDIGTNSVGWAVITDEYKVPSKKFKVLGNTDRHSIKKNLIGALLFDSGETAEATRLKRTAR | 41 |
| (Met minus) | RRYTRRKNRICYLQEIFSNEMAKVDDSFFHRLEESFLVEEDKKHERHPIFGNIVDEVAYHEKYPTIYH | |
| Streptococcus | LRKKLVDSTDKADLRLIYLALAHMIKFRGHFLIEGDLNPDNSDVDKLFIQLVQTYNQLFEENPINASG | |
| pyogenes | VDAKAILSARLSKSRRLENLIAQLPGEKKNGLFGNLIALSLGLTPNFKSNFDLAEDAKLQLSKDTYDD | |
| Q99ZW2 Cas9 | DLDNLLAQIGDQYADLFLAAKNLSDAILLSDILRVNTEITKAPLSASMIKRYDEHHQDLTLLKALVRQ | |
| with H840X, | QLPEKYKEIFFDQSKNGYAGYIDGGASQEEFYKFIKPILEKMDGTEELLVKLNREDLLRKQRTFDNGS | |
| wherein X is | IPHQIHLGELHAILRRQEDFYPFLKDNREKIEKILTFRIPYYVGPLARGNSRFAWMTRKSEETITPWN | |
| any alternate | FEEVVDKGASAQSFIERMTNFDKNLPNEKVLPKHSLLYEYFTVYNELTKVKYVTEGMRKPAFLSGEQK | |
| amino acid | KAIVDLLFKTNRKVTVKQLKEDYFKKIECFDSVEISGVEDRFNASLGTYHDLLKIIKDKDFLDNEENE | |
| | DILEDIVLTLTLFEDREMIEERLKTYAHLFDDKVMKQLKRRRYTGWGRLSRKLINGIRDKQSGKTILD | |
| | FLKSDGFANRNFMQLIHDDSLTFKEDIQKAQVSGQGDSLHEHIANLAGSPAIKKGILQTVKVVDELVK | |
| | VMGRHKPENIVIEMARENQTTQKGQKNSRERMKRIEEGIKELGSQILKEHPVENTQLQNEKLYLYYLQ | |
| | NGRDMYVDQELDINRLSDYDVDXIVPQSFLKDDSIDNKVLTRSDKNRGKSDNVPSEEVVKKMKNYWRQ | |
| | LLNAKLITQRKFDNLTKAERGGLSELDKAGFIKRQLVETRQITKHVAQILDSRMNTKYDENDKLIREV | |
| | KVITLKSKLVSDFRKDFQFYKVREINNYHHAHDAYLNAVVGTALIKKYPKLESEFVYGDYKVYDVRKM | |
| | IAKSEQEIGKATAKYFFYSNIMNFFKTEITLANGEIRKRPLIETNGETGEIVWDKGRDFATVRKVLSM | |
| | PQVNIVKKTEVQTGGFSKESILPKRNSDKLIARKKDWDPKKYGGFDSPTVAYSVLVVAKVEKGKSKKL | |
| | KSVKELLGITIMERSSFEKNPIDFLEAKGYKEVKKDLIIKLPKYSLFELENGRKRMLASAGELQKGNE | |
| | LALPSKYVNFLYLASHYEKLKGSPEDNEQKQLFVEQHKHYLDEIIEQISEFSKRVILADANLDKVLSA | |
| | YNKHRDKPIREQAENIIHLFTLTNLGAPAAFKYFDTTIDRKRYTSTKEVLDATLIHQSITGLYETRID | |
| | LSQLGGD | |
| Cas9 nickase | DKKYSIGLDIGTNSVGWAVITDEYKVPSKKFKVLGNTDRHSIKKNLIGALLFDSGETAEATRLKRTAR | 42 |
| (Met minus) | RRYTRRKNRICYLQEIFSNEMAKVDDSFFHRLEESFLVEEDKKHERHPIFGNIVDEVAYHEKYPTIYH | |
| Streptococcus | LRKKLVDSTDKADLRLIYLALAHMIKFRGHFLIEGDLNPDNSDVDKLFIQLVQTYNQLFEENPINASG | |
| pyogenes | VDAKAILSARLSKSRRLENLIAQLPGEKKNGLFGNLIALSLGLTPNFKSNFDLAEDAKLQLSKDTYDD | |
| Q99ZW2 Cas9 | DLDNLLAQIGDQYADLFLAAKNLSDAILLSDILRVNTEITKAPLSASMIKRYDEHHQDLTLLKALVRQ | |
| with H840A, | QLPEKYKEIFFDQSKNGYAGYIDGGASQEEFYKFIKPILEKMDGTEELLVKLNREDLLRKQRTFDNGS | |
| wherein X is | IPHQIHLGELHAILRRQEDFYPFLKDNREKIEKILTFRIPYYVGPLARGNSRFAWMTRKSEETITPWN | |
| any alternate | FEEVVDKGASAQSFIERMTNFDKNLPNEKVLPKHSLLYEYFTVYNELTKVKYVTEGMRKPAFLSGEQK | |
| amino acid | KAIVDLLFKTNRKVTVKQLKEDYFKKIECFDSVEISGVEDRFNASLGTYHDLLKIIKDKDFLDNEENE | |
| | DILEDIVLTLTLFEDREMIEERLKTYAHLFDDKVMKQLKRRRYTGWGRLSRKLINGIRDKQSGKTILD | |
| | FLKSDGFANRNFMQLIHDDSLTFKEDIQKAQVSGQGDSLHEHIANLAGSPAIKKGILQTVKVVDELVK | |
| | VMGRHKPENIVIEMARENQTTQKGQKNSRERMKRIEEGIKELGSQILKEHPVENTQLQNEKLYLYYLQ | |
| | NGRDMYVDQELDINRLSDYDVDAIVPQSFLKDDSIDNKVLTRSDKNRGKSDNVPSEEVVKKMKNYWRQ | |
| | LLNAKLITQRKFDNLTKAERGGLSELDKAGFIKRQLVETRQITKHVAQILDSRMNTKYDENDKLIREV | |
| | KVITLKSKLVSDFRKDFQFYKVREINNYHHAHDAYLNAVVGTALIKKYPKLESEFVYGDYKVYDVRKM | |
| | IAKSEQEIGKATAKYFFYSNIMNFFKTEITLANGEIRKRPLIETNGETGEIVWDKGRDFATVRKVLSM | |
| | PQVNIVKKTEVQTGGFSKESILPKRNSDKLIARKKDWDPKKYGGFDSPTVAYSVLVVAKVEKGKSKKL | |
| | KSVKELLGITIMERSSFEKNPIDFLEAKGYKEVKKDLIIKLPKYSLFELENGRKRMLASAGELQKGNE | |
| | LALPSKYVNFLYLASHYEKLKGSPEDNEQKQLFVEQHKHYLDEIIEQISEFSKRVILADANLDKVLSA | |
| | YNKHRDKPIREQAENIIHLFTLTNLGAPAAFKYFDTTIDRKRYTSTKEVLDATLIHQSITGLYETRID | |
| | LSQLGGD | |
| Cas9 nickase | DKKYSIGLDIGTNSVGWAVITDEYKVPSKKFKVLGNTDRHSIKKNLIGALLFDSGETAEATRLKRTAR | 43 |
| (Met minus) | RRYTRRKNRICYLQEIFSNEMAKVDDSFFHRLEESFLVEEDKKHERHPIFGNIVDEVAYHEKYPTIYH | |
| Streptococcus | LRKKLVDSTDKADLRLIYLALAHMIKFRGHFLIEGDLNPDNSDVDKLFIQLVQTYNQLFEENPINASG | |
| pyogenes | VDAKAILSARLSKSRRLENLIAQLPGEKKNGLFGNLIALSLGLTPNFKSNFDLAEDAKLQLSKDTYDD | |
| Q99ZW2 Cas9 | DLDNLLAQIGDQYADLFLAAKNLSDAILLSDILRVNTEITKAPLSASMIKRYDEHHQDLTLLKALVRQ | |
| with R863X, | QLPEKYKEIFFDQSKNGYAGYIDGGASQEEFYKFIKPILEKMDGTEELLVKLNREDLLRKQRTFDNGS | |
| wherein X is | IPHQIHLGELHAILRRQEDFYPFLKDNREKIEKILTFRIPYYVGPLARGNSRFAWMTRKSEETITPWN | |
| any alternate | FEEVVDKGASAQSFIERMTNFDKNLPNEKVLPKHSLLYEYFTVYNELTKVKYVTEGMRKPAFLSGEQK | |
| amino acid | KAIVDLLFKTNRKVTVKQLKEDYFKKIECFDSVEISGVEDRFNASLGTYHDLLKIIKDKDFLDNEENE | |
| | DILEDIVLTLTLFEDREMIEERLKTYAHLFDDKVMKQLKRRRYTGWGRLSRKLINGIRDKQSGKTILD | |
| | FLKSDGFANRNFMQLIHDDSLTFKEDIQKAQVSGQGDSLHEHIANLAGSPAIKKGILQTVKVVDELVK | |
| | VMGRHKPENIVIEMARENQTTQKGQKNSRERMKRIEEGIKELGSQILKEHPVENTQLQNEKLYLYYLQ | |
| | NGRDMYVDQELDINRLSDYDVDHIVPQSFLKDDSIDNKVLTRSDKNXGKSDNVPSEEVVKKMKNYWRQ | |
| | LLNAKLITQRKFDNLTKAERGGLSELDKAGFIKRQLVETRQITKHVAQILDSRMNTKYDENDKLIREV | |
| | KVITLKSKLVSDFRKDFQFYKVREINNYHHAHDAYLNAVVGTALIKKYPKLESEFVYGDYKVYDVRKM | |
| | IAKSEQEIGKATAKYFFYSNIMNFFKTEITLANGEIRKRPLIETNGETGEIVWDKGRDFATVRKVLSM | |
| | PQVNIVKKTEVQTGGFSKESILPKRNSDKLIARKKDWDPKKYGGFDSPTVAYSVLVVAKVEKGKSKKL | |
| | KSVKELLGITIMERSSFEKNPIDFLEAKGYKEVKKDLIIKLPKYSLFELENGRKRMLASAGELQKGNE | |
| | LALPSKYVNFLYLASHYEKLKGSPEDNEQKQLFVEQHKHYLDEIIEQISEFSKRVILADANLDKVLSA | |
| | YNKHRDKPIREQAENIIHLFTLTNLGAPAAFKYFDTTIDRKRYTSTKEVLDATLIHQSITGLYETRID | |
| | LSQLGGD | |
| Cas9 nickase | DKKYSIGLDIGTNSVGWAVITDEYKVPSKKFKVLGNTDRHSIKKNLIGALLFDSGETAEATRLKRTAR | 44 |
| (Met minus) | RRYTRRKNRICYLQEIFSNEMAKVDDSFFHRLEESFLVEEDKKHERHPIFGNIVDEVAYHEKYPTIYH | |
| Streptococcus | LRKKLVDSTDKADLRLIYLALAHMIKFRGHFLIEGDLNPDNSDVDKLFIQLVQTYNQLFEENPINASG | |
| pyogenes | VDAKAILSARLSKSRRLENLIAQLPGEKKNGLFGNLIALSLGLTPNFKSNFDLAEDAKLQLSKDTYDD | |
| Q99ZW2 Cas9 | DLDNLLAQIGDQYADLFLAAKNLSDAILLSDILRVNTEITKAPLSASMIKRYDEHHQDLTLLKALVRQ | |
| with R863A, | QLPEKYKEIFFDQSKNGYAGYIDGGASQEEFYKFIKPILEKMDGTEELLVKLNREDLLRKQRTFDNGS | |
| wherein X is | IPHQIHLGELHAILRRQEDFYPFLKDNREKIEKILTFRIPYYVGPLARGNSRFAWMTRKSEETITPWN | |
| any alternate | FEEVVDKGASAQSFIERMTNFDKNLPNEKVLPKHSLLYEYFTVYNELTKVKYVTEGMRKPAFLSGEQK | |
| amino acid | KAIVDLLFKTNRKVTVKQLKEDYFKKIECFDSVEISGVEDRFNASLGTYHDLLKIIKDKDFLDNEENE | |
| | DILEDIVLTLTLFEDREMIEERLKTYAHLFDDKVMKQLKRRRYTGWGRLSRKLINGIRDKQSGKTILD | |
| | FLKSDGFANRNFMQLIHDDSLTFKEDIQKAQVSGQGDSLHEHIANLAGSPAIKKGILQTVKVVDELVK | |
| | VMGRHKPENIVIEMARENQTTQKGQKNSRERMKRIEEGIKELGSQILKEHPVENTQLQNEKLYLYYLQ | |
| | NGRDMYVDQELDINRLSDYDVDHIVPQSFLKDDSIDNKVLTRSDKNAGKSDNVPSEEVVKKMKNYWRQ | |
| | LLNAKLITQRKFDNLTKAERGGLSELDKAGFIKRQLVETRQITKHVAQILDSRMNTKYDENDKLIREV | |
| | KVITLKSKLVSDFRKDFQFYKVREINNYHHAHDAYLNAVVGTALIKKYPKLESEFVYGDYKVYDVRKM | |
| | IAKSEQEIGKATAKYFFYSNIMNFFKTEITLANGEIRKRPLIETNGETGEIVWDKGRDFATVRKVLSM | |
| | PQVNIVKKTEVQTGGFSKESILPKRNSDKLIARKKDWDPKKYGGFDSPTVAYSVLVVAKVEKGKSKKL | |
| | KSVKELLGITIMERSSFEKNPIDFLEAKGYKEVKKDLIIKLPKYSLFELENGRKRMLASAGELQKGNE | |
| | LALPSKYVNFLYLASHYEKLKGSPEDNEQKQLFVEQHKHYLDEIIEQISEFSKRVILADANLDKVLSA | |
| | YNKHRDKPIREQAENIIHLFTLTNLGAPAAFKYFDTTIDRKRYTSTKEVLDATLIHQSITGLYETRID | |
| | LSQLGGD | |

Small-sized Cas9 variants

| | | |
|---|---|---|
| SaCas9 | MGKRNYILGLDIGITSVGYGIIDYETRDVIDAGVRLFKEANVENNEGRRSKRGARRLKRRRRHRIQRV | 45 |
| Staphylococcus | KKLLFDYNLLTDHSELSGINPYEARVKGLSQKLSEEEFSAALLHLAKRRGVHNVNEVEEDTGNELSTK | |
| aureus | EQISRNSKALEEKYVAELQLERLKKDGEVRGSINRFKTSDYVKEAKQLLKVQKAYHQLDQSFIDTYID | |
| 1053 AA | LLETRRTYYEGPGEGSPFGWKDIKEWYEMLMGHCTYFPEELRSVKYAYNADLYNALNDLNNLVITRDE | |
| 123 kDa | NEKLEYYEKFQIIENVFKQKKKPTLKQIAKEILVNEEDIKGYRVTSTGKPEFTNLKVYHDIKDITARK | |
| | EIIENAELLDQIAKILTIYQSSEDIQEELTNLNSELTQEEIEQISNLKGYTGTHNLSLKAINLILDEL | |
| | WHTNDNQIAIFNRLKLVPKKVDLSQQKEIPTTLVDDFILSPVVKRSFIQSIKVINAIIKKYGLPNDII | |
| | IELAREKNSKDAQKMINEMQKRNRQTNERIEEIIRTTGKENAKYLIEKIKLHDMQEGKCLYSLEAIPL | |
| | EDLLNNPFNYEVDHIIPRSVSFDNSFNNKVLVKQEENSKKGNRTPFQYLSSSDSKISYETFKKHILNL | |
| | AKGKGRISKTKKEYLLEERDINRFSVQKDFINRNLVDTRYATRGLMNLLRSYFRVNNLDVKVKSINGG | |
| | FTSFLRRKWKFKKERNKGYKHHAEDALIIANADFIFKEWKKLDKAKKVMENQMFEEKQAESMPEIETE | |
| | QEYKEIFITPHQIKHIKDFKDYKYSHRVDKKPNRKLINDTLYSTRKDDKGNTLIVNNLNGLYDKDNDK | |
| | LKKLINKSPEKLLMYHHDPQTYQKLKLIMEQYGDEKNPLYKYYEETGNYLTKYSKKDNGPVIKKIKYY | |
| | GNKLNAHLDITDDYPNSRNKVVKLSLKPYRFDVYLDNGVYKFVTVKNLDVIKKENYYEVNSKCYEEAK | |
| | KLKKISNQAEFIASFYKNDLIKINGELYRVIGVNNDLLNRIEVNMIDITYREYLENMNDKRPPHIIKT | |
| | IASKTQSIKKYSTDILGNLYEVKSKKHPQIIKK | |
| NmeCas 9 | MAAFKPNSINYILGLDIGIASVGWAMVEIDEEENPIRLIDLGVRVFERAEVPKTGDSLAMARRLARSV | 46 |
| N. | RRLTRRRAHRLLRTRRLLKREGVLQAANFDENGLIKSLPNTPWQLRAAALDRKLTPLEWSAVLLHLIK | |
| meningitidis | HRGYLSQRKNEGETADKELGALLKGVAGNAHALQTGDFRTPAELALNKFEKESGHIRNQRSDYSHTFS | |
| 1083 AA | RKDLQAELILLFEKQKEFGNPHVSGGLKEGIETLLMTQRPALSGDAVQKMLGHCTFEPAEPKAAKNTY | |
| 124.5 kDa | TAERFIWLTKLNNLRILEQGSERPLTDTERATLMDEPYRKSKLTYAQARKLLGLEDTAFFKGLRYGKD | |
| | NAEASTLMEMKAYHAISRALEKEGLKDKKSPLNLSPELQDEIGTAFSLFKTDEDITGRLKDRIQPEIL | |
| | EALLKHISFDKFVQISLKALRRIVPLMEQGKRYDEACAEIYGDHYGKKNTEEKIYLPPIPADEIRNPV | |
| | VLRALSQARKVINGVVRRYGSPARIHIETAREVGKSFKDRKEIEKRQEENRKDREKAAAKFREYFPNF | |
| | VGEPKSKDILKLRLYEQQHGKCLYSGKEINLGRLNEKGYVEIDAALPFSRTWDDSFNNKVLVLGSENQ | |
| | NKGNQTPYEYFNGKDNSREWQEFKARVETSRFPRSKKQRILLQKFDEDGFKERNLNDTRYVNRFLCQF | |
| | VADRMRLTGKGKKRVFASNGQITNLLRGFWGLRKVRAENDRHHALDAVVVACSTVAMQQKITRFVRYK | |
| | EMNAFDGKTIDKETGEVLHQKTHFPQPWEFFAQEVMIRVFGKPDGKPEFEEADTLEKLRTLLAEKLSS | |
| | RPEAVHEYVTPLFVSRAPNRKMSGQGHMETVKSAKRLDEGVSVLRVPLTQLKLKDLEKMVNREREPKL | |
| | YEALKARLEAHKDDPAKAFAEPFYKYDKAGNRTQQVKAVRVEQVQKTGVWVRNHNGIADNATMVRVDV | |
| | FEKGDKYYLVPIYSWQVAKGILPDRAVVQGKDEEDWQLIDDSFNFKFSLHPNDLVEVITKKARMFGYF | |
| | ASCHRGTGNINIRIHDLDHKIGKNGILEGIGVKTALSFQKYQIDELGKEIRPCRLKKRPPVR | |
| CjCas9 | MARILAFDIGISSIGWAFSENDELKDCGVRIFTKVENPKTGESLALPRRLARSARKRLARRKARLNHL | 47 |
| C. jejuni | KHLIANEFKLNYEDYQSFDESLAKAYKGSLISPYELRFRALNELLSKQDFARVILHIAKRRGYDDIKN | |
| 984 AA | SDDKEKGAILKAIKQNEEKLANYQSVGEYLYKEYFQKFKENSKEFTNVRNKKESYERCIAQSFLKDEL | |
| 114.9 kDa | KLIFKKQREFGFSFSKKFEEEVLSVAFYKRALKDFSHLVGNCSFFTDEKRAPKNSPLAFMFVALTRII | |
| | NLLNNLKNTEGILYTKDDLNALLNEVLKNGTLTYKQTKKLLGLSDDYEFKGEKGTYFIEFKKYKEFIK | |
| | ALGEHNLSQDDLNEIAKDITLIKDEIKLKKALAKYDLNQNQIDSLSKLEFKDHLNISFKALKLVTPLM | |
| | LEGKKYDEACNELNLKVAINEDKKDFLPAFNETYYKDEVTNPVVLRAIKEYRKVLNALLKKYGKVHKI | |
| | NIELAREVGKNHSQRAKIEKEQNENYKAKKDAELECEKLGLKINSKNILKLRLFKEQKEFCAYSGEKI | |
| | KISDLQDEKMLEIDHIYPYSRSFDDSYMNKVLVFTKQNQEKLNQTPFEAFGNDSAKWQKIEVLAKNLP | |
| | TKKQKRILDKNYKDKEQKNFKDRNLNDTRYIARLVLNYTKDYLDFLPLSDDENTKLNDTQKGSKVHVE | |
| | AKSGMLTSALRHTWGFSAKDRNNHLHHAIDAVIIAYANNSIVKAFSDFKKEQESNSAELYAKKISELD | |
| | YKNKRKFFEPFSGFRQKVLDKIDEIFVSKPERKKPSGALHEETFRKEEEFYQSYGGKEGVLKALELGK | |
| | IRKVNGKIVKNGDMFRVDIFKHKKTNKFYAVPIYTMDFALKVLPNKAVARSKKGEIKDWILMDENYEF | |
| | CFSLYKDSLILIQTKDMQEPEFVYYNAFTSSTVSLIVSKHDNKFETLSKNQKILFKNANEKEVIAKSI | |
| | GIQNLKVFEKYIVSALGEVTKAEFRQREDFKK | |
| GeoCas 9 | MRYKIGLDIGITSVGWAVMNLDIPRIEDLGVRIFDRAENPQTGESLALPRRLARSARRRLRRRKHRLE | 48 |
| G. | RIRRLVIREGILTKEELDKLFEEKHEIDVWQLRVEALDRKLNNDELARVLLHLAKRRGFKSNRKSERS | |
| stearo- | NKENSTMLKHIEENRAILSSYRTVGEMIVKDPKFALHKRNKGENYTNTIARDDLEREIRLIFSKQREF | |
| thermophilus | GNMSCTEEFENEYITIWASQRPVASKDDIEKKVGFCTFEPKEKRAPKATYTFQSFIAWEHINKLRLIS | |
| 1087 AA | PSGARGLTDEERRLLYEQAFQKNKITYHDIRTLLHLPDDTYFKGIVYDRGESRKQNENIRFLELDAYH | |
| 127 kDa | QIRKAVDKVYGKGKSSSFLPIDFDTFGYALTLFKDDADIHSYLRNEYEQNGKRMPNLANKVYDNELIE | |
| | ELLNLSFTKFGHLSLKALRSILPYMEQGEVYSSACERAGYTFTGPKKKQKTMLLPNIPPIANPVVMRA | |
| | LTQARKVVNAIIKKYGSPVSIHIELARDLSQTFDERRKTKKEQDENRKKNETAIRQLMEYGLTLNPTG | |
| | HDIVKFKLWSEQNGRCAYSLQPIEIERLLEPGYVEVDHVIPYSRSLDDSYTNKVLVLTRENREKGNRI | |
| | PAEYLGVGTERWQQFETFVLTNKQFSKKKRDRLLRLHYDENEETEFKNRNLNDTRYISRFFANFIREH | |
| | LKFAESDDKQKVYTVNGRVTAHLRSRWEFNKNREESDLHHAVDAVIVACTTPSDIAKVTAFYQRREQN | |
| | KELAKKTEPHFPQPWPHFADELRARLSKHPKESIKALNLGNYDDQKLESLQPVFVSRMPKRSVTGAAH | |
| | QETLRRYVGIDERSGKIQTVVKTKLSEIKLDASGHFPMYGKESDPRTYEAIRQRLLEHNNDPKKAFQE | |
| | PLYKPKKNGEPGPVIRTVKIIDTKNQVIPLNDGKTVAYNSNIVRVDVFEKDGKYYCVPVYTMDIMKGI | |
| | LPNKAIEPNKPYSEWKEMTEDYTFRFSLYPNDLIRIELPREKTVKTAAGEEINVKDVFVYYKTIDSAN | |
| | GGLELISHDHRFSLRGVGSRTLKRFEKYQVDVLGNIYKVRGEKRVGLASSAHSKPGKTIRPLQSTRD | |
| LbaCas12a | MSKLEKFTNCYSLSKTLRFKAIPVGKTQENIDNKRLLVEDEKRAEDYKGVKKLLDRYYLSFINDVLHS | 49 |
| L. bacterium | IKLKNLNNYISLFRKKTRTEKENKELENLEINLRKEIAKAFKGNEGYKSLFKKDIIETILPEFLDDKD | |
| 1228 AA | EIALVNSFNGFTTAFTGFFDNRENMFSEEAKSTSIAFRCINENLTRYISNMDIFEKVDAIFDKHEVQE | |
| 143.9 kDa | IKEKILNSDYDVEDFFEGEFFNFVLTQEGIDVYNAIIGGFVTESGEKIKGLNEYINLYNQKTKQKLPK | |
| | FKPLYKQVLSDRESLSFYGEGYTSDEEVLEVFRNTLNKNSEIFSSIKKLEKLFKNFDEYSSAGIFVKN | |
| | GPAISTISKDIFGEWNVIRDKWNAEYDDIHLKKKAVVTEKYEDDRRKSFKKIGSFSLEQLQEYADADL | |
| | SVVEKLKEIIIQKVDEIYKVYGSSEKLFDADFVLEKSLKKNDAVVAIMKDLLDSVKSFENYIKAFFGE | |
| | GKETNRDESFYGDFVLAYDILLKVDHIYDAIRNYVTQKPYSKDKFKLYFQNPQFMGGWDKDKETDYRA | |
| | TILRYGSKYYLAIMDKKYAKCLQKIDKDDVNGNYEKINYKLLPGPNKMLPKVFFSKKWMAYYNPSEDI | |
| | QKIYKNGTFKKGDMFNLNDCHKLIDFFKDSISRYPKWSNAYDFNFSETEKYKDIAGFYREVEEQGYKV | |
| | SFESASKKEVDKLVEEGKLYMFQIYNKDFSDKSHGTPNLHTMYFKLLFDENNHGQIRLSGGAELFMRR | |
| | ASLKKEELVVHPANSPIANKNPDNPKKTTTLSYDVYKDKRFSEDQYELHIPIAINKCPKNIFKINTEV | |
| | RVLLKHDDNPYVIGIDRGERNLLYIVVVDGKGNIVEQYSLNEIINNFNGIRIKTDYHSLLDKKEKERF | |
| | EARQNWTSIENIKELKAGYISQVVHKICELVEKYDAVIALEDLNSGFKNSRVKVEKQVYQKFEKMLID | |
| | KLNYMVDKKSNPCATGGALKGYQITNKFESFKSMSTQNGFIFYIPAWLTSKIDPSTGFVNLLKTKYTS | |
| | IADSKKFISSFDRIMYVPEEDLFEFALDYKNFSRTDADYIKKWKLYSYGNRIRIFRNPKKNNVFDWEE | |
| | VCLTSAYKELFNKYGINYQQGDIRALLCEQSDKAFYSSFMALMSLMLQMRNSITGRTDVDFLISPVKN | |
| | SDGIFYDSRNYEAQENAILPKNADANGAYNIARKVLWAIGQFKKAEDEKLDKVKIAISNKEWLEYAQT | |
| | SVKH | |
| BhCas12b | MATRSFILKIEPNEEVKKGLWKTHEVLNHGIAYYMNILKLIRQEAIYEHHEQDPKNPKKVSKAEIQAE | 50 |
| B. hisashii | LWDFVLKMQKCNSFTHEVDKDEVFNILRELYEELVPSSVEKKGEANQLSNKFLYPLVDPNSQSGKGTA | |
| 1108 AA | SSGRKPRWYNLKIAGDPSWEEEKKKWEEDKKKDPLAKILGKLAEYGLIPLFIPYTDSNEPIVKEIKWM | |
| 130.4 kDa | EKSRNQSVRRLDKDMFIQALERFLSWESWNLKVKEEYEKVEKEYKTLEERIKEDIQALKALEQYEKER | |
| | QEQLLRDTLNTNEYRLSKRGLRGWREIIQKWLKMDENEPSEKYLEVFKDYQRKHPREAGDYSVYEFLS | |
| | KKENHFIWRNHPEYPYLYATFCEIDKKKKDAKQQATFTLADPINHPLWVRFEERSGSNLNKYRILTEQ | |
| | LHTEKLKKKLTVQLDRLIYPTESGGWEEKGKVDIVLLPSRQFYNQIFLDIEEKGKHAFTYKDESIKFP | |
| | LKGTLGGARVQFDRDHLRRYPHKVESGNVGRIYFNMTVNIEPTESPVSKSLKIHRDDFPKVVNFKPKE | |
| | LTEWIKDSKGKKLKSGIESLEIGLRVMSIDLGQRQAAAASIFEVVDQKPDIEGKLFFPIKGTELYAVH | |
| | RASFNIKLPGETLVKSREVLRKAREDNLKLMNQKLNFLRNVLHFQQFEDITEREKRVTKWISRQENSD | |
| | VPLVYQDELIQIRELMYKPYKDWVAFLKQLHKRLEVEIGKEVKHWRKSLSDGRKGLYGISLKNIDEID | |
| | RTRKFLLRWSLRPTEPGEVRRLEPGQRFAIDQLNHLNALKEDRLKKMANTIIMHALGYCYDVRKKKWQ | |
| | AKNPACQIILFEDLSNYNPYEERSRFENSKLMKWSRREIPRQVALQGEIYGLQVGEVGAQFSSRFHAK | |
| | TGSPGIRCSVVTKEKLQDNRFFKNLQREGRLTLDKIAVLKEGDLYPDKGGEKFISLSKDRKCVTTHAD | |
| | INAAQNLQKRFWTRTHGFYKVYCKAYQVDGQTVYIPESKDQKQKIIEEFGEGYFILKDGVYEWVNAGK | |
| | LKIKKGSSKQSSSELVDSDILKDSFDLASELKGEKLMLYRDPSGNVFPSDKWMAAGVFFGKLERILIS | |
| | KLTNQYSISTIEDDSSKQSM | |

Cas9 equivalents

| | | |
|---|---|---|
| AsCas12a | MTQFEGFTNLYQVSKTLRFELIPQGKTLKHIQEQGFIEEDKARNDHYKELKPIIDRIYKTYADQCLQL | 51 |
| (previously | VQLDWENLSAAIDSYRKEKTEETRNALIEEQATYRNAIHDYFIGRTDNLTDAINKRHAEIYKGLFKAE | |
| known as Cpf1) | LFNGKVLKQLGTVTTTEHENALLRSFDKFTTYFSGFYENRKNVFSAEDISTAIPHRIVQDNFPKFKEN | |
| Acidaminococcus | CHIFTRLITAVPSLREHFENVKKAIGIFVSTSIEEVFSFPFYNQLLTQTQIDLYNQLLGGISREAGTE | |
| sp. (strain | KIKGLNEVLNLAIQKNDETAHIIASLPHRFIPLFKQILSDRNTLSFILEEFKSDEEVIQSFCKYKTLL | |
| BV3L6) | RNENVLETAEALFNELNSIDLTHIFISHKKLETISSALCDHWDTLRNALYERRISELTGKITKSAKEK | |
| UniProtKB | VQRSLKHEDINLQEIISAAGKELSEAFKQKTSEILSHAHAALDQPLPTTLKKQEEKEILKSQLDSLLG | |
| U2UMQ6 | LYHLLDWFAVDESNEVDPEFSARLTGIKLEMEPSLSFYNKARNYATKKPYSVEKFKLNFQMPTLASGW | |
| | DVNKEKNNGAILFVKNGLYYLGIMPKQKGRYKALSFEPTEKTSEGFDKMYYDYFPDAAKMIPKCSTQL | |
| | KAVTAHFQTHTTPILLSNNFIEPLEITKEIYDLNNPEKEPKKFQTAYAKKTGDQKGYREALCKWIDFT | |
| | RDFLSKYTKTTSIDLSSLRPSSQYKDLGEYYAELNPLLYHISFQRIAEKEIMDAVETGKLYLFQIYNK | |
| | DFAKGHHGKPNLHTLYWTGLFSPENLAKTSIKLNGQAELFYRPKSRMKRMAHRLGEKMLNKKLKDQKT | |
| | PIPDTLYQELYDYVNHRLSHDLSDEARALLPNVITKEVSHEIIKDRRFTSDKFFFHVPITLNYQAANS | |
| | PSKFNQRVNAYLKEHPETPIIGIDRGERNLIYITVIDSTGKILEQRSLNTIQQFDYQKKLDNREKERV | |
| | AARQAWSVVGTIKDLKQGYLSQVIHEIVDLMIHYQAVVVLENLNFGFKSKRTGIAEKAVYQQFEKMLI | |
| | DKLNCLVLKDYPAEKVGGVLNPYQLTDQFTSFAKMGTQSGFLFYVPAPYTSKIDPLTGFVDPFVWKTI | |
| | KNHESRKHFLEGFDFLHYDVKTGDFILHFKMNRNLSFQRGLPGFMPAWDIVFEKNETQFDAKGTPFIA | |
| | GKRIVPVIENHRFTGRYRDLYPANELIALLEEKGIVFRDGSNILPKLLENDDSHAIDTMVALIRSVLQ | |
| | MRNSNAATGEDYINSPVRDLNGVCFDSRFQNPEWPMDADANGAYHIALKGQLLLNHLKESKDLKLQNG | |
| | ISNQDWLAYIQELRN | |
| AsCas12a | MTQFEGFTNLYQVSKTLRFELIPQGKTLKHIQEQGFIEEDKARNDHYKELKPIIDRIYKTYADQCLQL | 52 |
| nickase (e.g., | VQLDWENLSAAIDSYRKEKTEETRNALIEEQATYRNAIHDYFIGRTDNLTDAINKRHAEIYKGLFKAE | |
| R1226A) | LFNGKVLKQLGTVTTTEHENALLRSFDKFTTYFSGFYENRKNVFSAEDISTAIPHRIVQDNFPKFKEN | |
| | CHIFTRLITAVPSLREHFENVKKAIGIFVSTSIEEVFSFPFYNQLLTQTQIDLYNQLLGGISREAGTE | |
| | KIKGLNEVLNLAIQKNDETAHIIASLPHRFIPLFKQILSDRNTLSFILEEFKSDEEVIQSFCKYKTLL | |
| | RNENVLETAEALFNELNSIDLTHIFISHKKLETISSALCDHWDTLRNALYERRISELTGKITKSAKEK | |
| | VQRSLKHEDINLQEIISAAGKELSEAFKQKTSEILSHAHAALDQPLPTTLKKQEEKEILKSQLDSLLG | |
| | LYHLLDWFAVDESNEVDPEFSARLTGIKLEMEPSLSFYNKARNYATKKPYSVEKFKLNFQMPTLASGW | |
| | DVNKEKNNGAILFVKNGLYYLGIMPKQKGRYKALSFEPTEKTSEGFDKMYYDYFPDAAKMIPKCSTQL | |
| | KAVTAHFQTHTTPILLSNNFIEPLEITKEIYDLNNPEKEPKKFQTAYAKKTGDQKGYREALCKWIDFT | |
| | RDFLSKYTKTTSIDLSSLRPSSQYKDLGEYYAELNPLLYHISFQRIAEKEIMDAVETGKLYLFQIYNK | |
| | DFAKGHHGKPNLHTLYWTGLFSPENLAKTSIKLNGQAELFYRPKSRMKRMAHRLGEKMLNKKLKDQKT | |
| | PIPDTLYQELYDYVNHRLSHDLSDEARALLPNVITKEVSHEIIKDRRFTSDKFFFHVPITLNYQAANS | |
| | PSKFNQRVNAYLKEHPETPIIGIDRGERNLIYITVIDSTGKILEQRSLNTIQQFDYQKKLDNREKERV | |
| | AARQAWSVVGTIKDLKQGYLSQVIHEIVDLMIHYQAVVVLENLNFGFKSKRTGIAEKAVYQQFEKMLI | |
| | DKLNCLVLKDYPAEKVGGVLNPYQLTDQFTSFAKMGTQSGFLFYVPAPYTSKIDPLTGFVDPFVWKTI | |
| | KNHESRKHFLEGFDFLHYDVKTGDFILHFKMNRNLSFQRGLPGFMPAWDIVFEKNETQFDAKGTPFIA | |
| | GKRIVPVIENHRFTGRYRDLYPANELIALLEEKGIVFRDGSNILPKLLENDDSHAIDTMVALIRSVLQ | |
| | MANSNAATGEDYINSPVRDLNGVCFDSRFQNPEWPMDADANGAYHIALKGQLLLNHLKESKDLKLQNG | |
| | ISNQDWLAYIQELRN | |
| LbCas12a | MNYKTGLEDFIGKESLSKTLRNALIPTESTKIHMEEMGVIRDDELRAEKQQELKEIMDDYYRTFIEEK | 53 |
| (previously | LGQIQGIQWNSLFQKMEETMEDISVRKDLDKIQNEKRKEICCYFTSDKRFKDLFNAKLITDILPNFIK | |
| known as Cpf1) | DNKEYTEEEKAEKEQTRVLFQRFATAFTNYFNQRRNNFSEDNISTAISFRIVNENSEIHLQNMRAFQR | |
| Lachnospiraceae | IEQQYPEEVCGMEEEYKDMLQEWQMKHIYSVDFYDRELTQPGIEYYNGICGKINEHMNQFCQKNRINK | |
| bacterium | NDFRMKKLHKQILCKKSSYYEIPFRFESDQEVYDALNEFIKTMKKKEIIRRCVHLGQECDDYDLGKIY | |
| GAM79 | ISSNKYEQISNALYGSWDTIRKCIKEEYMDALPGKGEKKEEKAEAAAKKEEYRSIADIDKIISLYGSE | |
| Ref Seq. | MDRTISAKKCITEICDMAGQISIDPLVCNSDIKLLQNKEKTTEIKTILDSFLHVYQWGQTFIVSDIIE | |
| WP_119623382.1 | KDSYFYSELEDVLEDFEGITTLYNHVRSYVTQKPYSTVKFKLHFGSPTLANGWSQSKEYDNNAILLMR | |
| | DQKFYLGIFNVRNKPDKQIIKGHEKEEKGDYKKMIYNLLPGPSKMLPKVFITSRSGQETYKPSKHILD | |
| | GYNEKRHIKSSPKFDLGYCWDLIDYYKECIHKHPDWKNYDFHFSDTKDYEDISGFYREVEMQGYQIKW | |
| | TYISADEIQKLDEKGQIFLFQIYNKDFSVHSTGKDNLHTMYLKNLFSEENLKDIVLKLNGEAELFFRK | |
| | ASIKTPIVHKKGSVLVNRSYTQTVGNKEIRVSIPEEYYTEIYNYLNHIGKGKLSSEAQRYLDEGKIKS | |
| | FTATKDIVKNYRYCCDHYFLHLPITINFKAKSDVAVNERTLAYIAKKEDIHIIGIDRGERNLLYISVV | |
| | DVHGNIREQRSFNIVNGYDYQQKLKDREKSRDAARKNWEEIEKIKELKEGYLSMVIHYIAQLVVKYNA | |
| | VVAMEDLNYGFKTGRFKVERQVYQKFETMLIEKLHYLVFKDREVCEEGGVLRGYQLTYIPESLKKVGK | |
| | QCGFIFYVPAGYTSKIDPTTGFVNLFSFKNLTNRESRQDFVGKFDEIRYDRDKKMFEFSFDYNNYIKK | |
| | GTILASTKWKVYTNGTRLKRIVVNGKYTSQSMEVELTDAMEKMLQRAGIEYHDGKDLKGQIVEKGIEA | |
| | EIIDIFRLTVQMRNSRSESEDREYDRLISPVLNDKGEFFDTATADKTLPQDADANGAYCIALKGLYEV | |
| | KQIKENWKENEQFPRNKLVQDNKTWFDFMQKKRYL | |
| PcCas12a- | MAKNFEDFKRLYSLSKTLRFEAKPIGATLDNIVKSGLLDEDEHRAASYVKVKKLIDEYHKVFIDRVLD | 54 |
| previously | DGCLPLENKGNNNSLAEYYESYVSRAQDEDAKKKFKEIQQNLRSVIAKKLTEDKAYANLFGNKLIESY | |
| known at Cpf1 | KDKEDKKKIIDSDLIQFINTAESTQLDSMSQDEAKELVKEFWGFVTYFYGFFDNRKNMYTAEEKSTGI | |
| Prevotella | AYRLVNENLPKFIDNIEAFNRAITRPEIQENMGVLYSDFSEYLNVESIQEMFQLDYYNMLLTQKQIDV | |
| copri | YNAIIGGKTDDEHDVKIKGINEYINLYNQQHKDDKLPKLKALFKQILSDRNAISWLPEEFNSDQEVLN | |
| Ref Seq. | AIKDCYERLAENVLGDKVLKSLLGSLADYSLDGIFIRNDLQLTDISQKMFGNWGVIQNAIMQNIKRVA | |
| WP_119227726.1 | PARKHKESEEDYEKRIAGIFKKADSFSISYINDCLNEADPNNAYFVENYFATFGAVNTPTMQRENLFA | |
| | LVQNAYTEVAALLHSDYPTVKHLAQDKANVSKIKALLDAIKSLQHFVKPLLGKGDESDKDERFYGELA | |
| | SLWAELDTVTPLYNMIRNYMTRKPYSQKKIKLNFENPQLLGGWDANKEKDYATIILRRNGLYYLAIMD | |
| | KDSRKLLGKAMPSDGECYEKMVYKFFKDVTTMIPKCSTQLKDVQAYFKVNTDDYVLNSKAFNKPLTIT | |
| | KEVFDLNNVLYGKYKKFQKGYLTATGDNVGYTHAVNVWIKFCMDFLNSYDSTCIYDFSSLKPESYLSL | |
| | DAFYQDANLLLYKLSFARASVSYINQLVEEGKMYLFQIYNKDFSEYSKGTPNMHTLYWKALFDERNLA | |
| | DVVYKLNGQAEMFYRKKSIENTHPTHPANHPILNKNKDNKKKESLFDYDLIKDRRYTVDKFMFHVPIT | |
| | MNFKSVGSENINQDVKAYLRHADDMHIIGIDRGERHLLYLVVIDLQGNIKEQYSLNEIVNEYNGNTYH | |
| | TNYHDLLDVREEERLKARQSWQTIENIKELKEGYLSQVIHKITQLMVRYHAIVVLEDLSKGFMRSRQK | |
| | VEKQVYQKFEKMLIDKLNYLVDKKTDVSTPGGLLNAYQLTCKSDSSQKLGKQSGFLFYIPAWNTSKID | |
| | PVTGFVNLLDTHSLNSKEKIKAFFSKFDAIRYNKDKKWFEFNLDYDKFGKKAEDTRTKWTLCTRGMRI | |
| | DTFRNKEKNSQWDNQEVDLTTEMKSLLEHYYIDIHGNLKDAISAQTDKAFFTGLLHILKLTLQMRNSI | |
| | TGTETDYLVSPVADENGIFYDSRSCGNQLPENADANGAYNIARKGLMLIEQIKNAEDLNNVKFDISNK | |
| | AWLNFAQQKPYKNG | |
| ErCas12a- | MFSAKLISDILPEFVIHNNNYSASEKEEKTQVIKLFSRFATSFKDYFKNRANCFSANDISSSSCHRIV | 55 |
| previously | NDNAEIFFSNALVYRRIVKNLSNDDINKISGDMKDSLKEMSLEEIYSYEKYGEFITQEGISFYNDICG | |
| known at Cpf1 | KVNLFMNLYCQKNKENKNLYKLRKLHKQILCIADTSYEVPYKFESDEEVYQSVNGFLDNISSKHIVER | |
| Eubacterium | LRKIGENYNGYNLDKIYIVSKFYESVSQKTYRDWETINTALEIHYNNILPGNGKSKADKVKKAVKNDL | |
| rectale | QKSITEINELVSNYKLCPDDNIKAETYIHEISHILNNFEAQELKYNPEIHLVESELKASELKNVLDVI | |
| Ref Seq. | MNAFHWCSVFMTEELVDKDNNFYAELEEIYDEIYPVISLYNLVRNYVTQKPYSTKKIKLNFGIPTLAD | |
| WP_119223642.1 | GWSKSKEYSNNAIILMRDNLYYLGIFNAKNKPDKKIIEGNTSENKGDYKKMIYNLLPGPNKMIPKVFL | |
| | SSKTGVETYKPSAYILEGYKQNKHLKSSKDFDITFCHDLIDYFKNCIAIHPEWKNFGFDFSDTSTYED | |
| | ISGFYREVELQGYKIDWTYISEKDIDLLQEKGQLYLFQIYNKDFSKKSSGNDNLHTMYLKNLFSEENL | |
| | KDIVLKLNGEAEIFFRKSSIKNPIIHKKGSILVNRTYEAEEKDQFGNIQIVRKTIPENIYQELYKYFN | |
| | DKSDKELSDEAAKLKNVVGHHEAATNIVKDYRYTYDKYFLHMPITINFKANKTSFINDRILQYIAKEK | |
| | DLHVIGIDRGERNLIYVSVIDTCGNIVEQKSFNIVNGYDYQIKLKQQEGARQIARKEWKEIGKIKEIK | |
| | EGYLSLVIHEISKMVIKYNAIIAMEDLSYGFKKGRFKVERQVYQKFETMLINKLNYLVFKDISITENG | |
| | GLLKGYQLTYIPDKLKNVGHQCGCIFYVPAAYTSKIDPTTGFVNIFKFKDLTVDAKREFIKKFDSIRY | |
| | DSDKNLFCFTFDYNNFITQNTVMSKSSWSVYTYGVRIKRRFVNGRFSNESDTIDITKDMEKTLEMTDI | |
| | NWRDGHDLRQDIIDYEIVQHIFEIFKLTVQMRNSLSELEDRDYDRLISPVLNENNIFYDSAKAGDALP | |
| | KDADANGAYCIALKGLYEIKQITENWKEDGKFSRDKLKISNKDWFDFIQNKRYL | |
| CsCas12a- | MNYKTGLEDFIGKESLSKTLRNALIPTESTKIHMEEMGVIRDDELRAEKQQELKEIMDDYYRAFIEEK | 56 |
| previously | LGQIQGIQWNSLFQKMEETMEDISVRKDLDKIQNEKRKEICCYFTSDKRFKDLFNAKLITDILPNFIK | |
| known at Cpf1 | DNKEYTEEEKAEKEQTRVLFQRFATAFTNYFNQRRNNFSEDNISTAISFRIVNENSEIHLQNMRAFQR | |
| Clostridium | IEQQYPEEVCGMEEEYKDMLQEWQMKHIYLVDFYDRVLTQPGIEYYNGICGKINEHMNQFCQKNRINK | |
| sp. AF34-10BH | NDFRMKKLHKQILCKKSSYYEIPFRFESDQEVYDALNEFIKTMKEKEIICRCVHLGQKCDDYDLGKIY | |
| Ref Seq. | ISSNKYEQISNALYGSWDTIRKCIKEEYMDALPGKGEKKEEKAEAAAKKEEYRSIADIDKIISLYGSE | |
| WP_118538418.1 | MDRTISAKKCITEICDMAGQISTDPLVCNSDIKLLQNKEKTTEIKTILDSFLHVYQWGQTFIVSDIIE | |
| | KDSYFYSELEDVLEDFEGITTLYNHVRSYVTQKPYSTVKFKLHFGSPTLANGWSQSKEYDNNAILLMR | |
| | DQKFYLGIFNVRNKPDKQIIKGHEKEEKGDYKKMIYNLLPGPSKMLPKVFITSRSGQETYKPSKHILD | |
| | GYNEKRHIKSSPKFDLGYCWDLIDYYKECIHKHPDWKNYDFHFSDTKDYEDISGFYREVEMQGYQIKW | |
| | TYISADEIQKLDEKGQIFLFQIYNKDFSVHSTGKDNLHTMYLKNLFSEENLKDIVLKLNGEAELFFRK | |
| | ASIKTPVVHKKGSVLVNRSYTQTVGDKEIRVSIPEEYYTEIYNYLNHIGRGKLSTEAQRYLEERKIKS | |
| | FTATKDIVKNYRYCCDHYFLHLPITINFKAKSDIAVNERTLAYIAKKEDIHIIGIDRGERNLLYISVV | |
| | DVHGNIREQRSFNIVNGYDYQQKLKDREKSRDAARKNWEEIEKIKELKEGYLSMVIHYIAQLVVKYNA | |
| | VVAMEDLNYGFKTGRFKVERQVYQKFETMLIEKLHYLVFKDREVCEEGGVLRGYQLTYIPESLKKVGK | |
| | QCGFIFYVPAGYTSKIDPTTGFVNLFSFKNLTNRESRQDFVGKFDEIRYDRDKKMFEFSFDYNNYIKK | |
| | GTMLASTKWKVYTNGTRLKRIWNGKYTSQSMEVELTDAMEKMLQRAGIEYHDGKDLKGQIVEKGIEA | |
| | EIIDIFRLTVQMRNSRSESEDREYDRLISPVLNDKGEFFDTATADKTLPQDADANGAYCIALKGLYEV | |
| | KQIKENWKENEQFPRNKLVQDNKTWFDFMQKKRYL | |
| BhCas12b | MATRSFILKIEPNEEVKKGLWKTHEVLNHGIAYYMNILKLIRQEAIYEHHEQDPKNPKKVSKAEIQAE | 57 |
| Bacillus | LWDFVLKMQKCNSFTHEVDKDEVFNILRELYEELVPSSVEKKGEANQLSNKFLYPLVDPNSQSGKGTA | |
| hisashii | SSGRKPRWYNLKIAGDPSWEEEKKKWEEDKKKDPLAKILGKLAEYGLIPLFIPYTDSNEPIVKEIKWM | |
| Ref Seq. | EKSRNQSVRRLDKDMFIQALERFLSWESWNLKVKEEYEKVEKEYKTLEERIKEDIQALKALEQYEKER | |
| WP_095142515.1 | QEQLLRDTLNTNEYRLSKRGLRGWREIIQKWLKMDENEPSEKYLEVFKDYQRKHPREAGDYSVYEFLS | |
| | KKENHFIWRNHPEYPYLYATFCEIDKKKKDAKQQATFTLADPINHPLWVRFEERSGSNLNKYRILTEQ | |
| | LHTEKLKKKLTVQLDRLIYPTESGGWEEKGKVDIVLLPSRQFYNQIFLDIEEKGKHAFTYKDESIKFP | |
| | LKGTLGGARVQFDRDHLRRYPHKVESGNVGRIYFNMTVNIEPTESPVSKSLKIHRDDFPKVVNFKPKE | |
| | LTEWIKDSKGKKLKSGIESLEIGLRVMSIDLGQRQAAAASIFEVVDQKPDIEGKLFFPIKGTELYAVH | |
| | RASFNIKLPGETLVKSREVLRKAREDNLKLMNQKLNFLRNVLHFQQFEDITEREKRVTKWISRQENSD | |
| | VPLVYQDELIQIRELMYKPYKDWVAFLKQLHKRLEVEIGKEVKHWRKSLSDGRKGLYGISLKNIDEID | |
| | RTRKFLLRWSLRPTEPGEVRRLEPGQRFAIDQLNHLNALKEDRLKKMANTIIMHALGYCYDVRKKKWQ | |
| | AKNPACQIILFEDLSNYNPYEERSRFENSKLMKWSRREIPRQVALQGEIYGLQVGEVGAQFSSRFHAK | |
| | TGSPGIRCSVVTKEKLQDNRFFKNLQREGRLTLDKIAVLKEGDLYPDKGGEKFISLSKDRKCVTTHAD | |
| | INAAQNLQKRFWTRTHGFYKVYCKAYQVDGQTVYIPESKDQKQKIIEEFGEGYFILKDGVYEWVNAGK | |
| | LKIKKGSSKQSSSELVDSDILKDSFDLASELKGEKLMLYRDPSGNVFPSDKWMAAGVFFGKLERILIS | |
| | KLTNQYSISTIEDDSSKQSM | |
| ThCas12b | MSEKTTQRAYTLRLNRASGECAVCQNNSCDCWHDALWATHKAVNRGAKAFGDWLLTLRGGLCHTLVEM | 58 |
| Thermomonas | EVPAKGNNPPQRPTDQERRDRRVLLALSWLSVEDEHGAPKEFIVATGRDSADDRAKKVEEKLREILEK | |
| hydrothermalis | RDFQEHEIDAWLQDCGPSLKAHIREDAVWVNRRALFDAAVERIKTLTWEEAWDFLEPFFGTQYFAGIG | |
| Ref Seq. | DGKDKDDAEGPARQGEKAKDLVQKAGQWLSARFGIGTGADFMSMAEAYEKIAKWASQAQNGDNGKATI | |
| WP_072754838 | EKLACALRPSEPPTLDTVLKCISGPGHKSATREYLKTLDKKSTVTQEDLNQLRKLADEDARNCRKKVG | |
| | KKGKKPWADEVLKDVENSCELTYLQDNSPARHREFSVMLDHAARRVSMAHSWIKKAEQRRRQFESDAQ | |
| | KLKNLQERAPSAVEWLDRFCESRSMTTGANTGSGYRIRKRAIEGWSYVVQAWAEASCDTEDKRIAAAR | |
| | KVQADPEIEKFGDIQLFEALAADEAICVWRDQEGTQNPSILIDYVTGKTAEHNQKRFKVPAYRHPDEL | |
| | RHPVFCDFGNSRWSIQFAIHKEIRDRDKGAKQDTRQLQNRHGLKMRLWNGRSMTDVNLHWSSKRLTAD | |
| | LALDQNPNPNPTEVTRADRLGRAASSAFDHVKIKNVFNEKEWNGRLQAPRAELDRIAKLEEQGKTEQA | |
| | EKLRKRLRWYVSFSPCLSPSGPFIVYAGQHNIQPKRSGQYAPHAQANKGRARLAQLILSRLPDLRILS | |
| | VDLGHRFAAACAVWETLSSDAFRREIQGLNVLAGGSGEGDLFLHVEMTGDDGKRRTVVYRRIGPDQLL | |
| | DNTPHPAPWARLDRQFLIKLQGEDEGVREASNEELWTVHKLEVEVGRTVPLIDRMVRSGFGKTEKQKE | |
| | RLKKLRELGWISAMPNEPSAETDEKEGEIRSISRSVDELMSSALGTLRLALKRHGNRARIAFAMTADY | |
| | KPMPGGQKYYFHEAKEASKNDDETKRRDNQIEFLQDALSLWHDLFSSPDWEDNEAKKLWQNHIATLPN | |
| | YQTPEEISAELKRVERNKKRKENRDKLRTAAKALAENDQLRQHLHDTWKERWESDDQQWKERLRSLKD | |
| | WIFPRGKAEDNPSIRHVGGLSITRINTISGLYQILKAFKMRPEPDDLRKNIPQKGDDELENFNRRLLE | |
| | ARDRLREQRVKQLASRIIEAALGVGRIKIPKNGKLPKRPRTTVDTPCHAVVIESLKTYRPDDLRTRRE | |
| | NRQLMQWSSAKVRKYLKEGCELYGLHFLEVPANYTSRQCSRTGLPGIRCDDVPTGDFLKAPWWRRAIN | |
| | TAREKNGGDAKDRFLVDLYDHLNNLQSKGEALPATVRVPRQGGNLFIAGAQLDDTNKERRAIQADLNA | |
| | AANIGLRALLDPDWRGRWWYVPCKDGTSEPALDRIEGSTAFNDVRSLPTGDNSSRRAPREIENLWRDP | |
| | SGDSLESGTWSPTRAYWDTVQSRVIELLRRHAGLPTS | |
| LsCas12b | MSIRSFKLKLKTKSGVNAEQLRRGLWRTHQLINDGIAYYMNWLVLLRQEDLFIRNKETNEIEKRSKEE | 59 |
| Laceyella | IQAVLLERVHKQQQRNQWSGEVDEQTLLQALRQLYEEIVPSVIGKSGNASLKARFFLGPLVDPNNKTT | |
| sacchari | KDVSKSGPTPKWKKMKDAGDPNWVQEYEKYMAERQTLVRLEEMGLIPLFPMYTDEVGDIHWLPQASGY | |
| WP_132221894.1 | TRTWDRDMFQQAIERLLSWESWNRRVRERRAQFEKKTHDFASRFSESDVQWMNKLREYEAQQEKSLEE | |
| | NAFAPNEPYALTKKALRGWERVYHSWMRLDSAASEEAYWQEVATCQTAMRGEFGDPAIYQFLAQKENH | |
| | DIWRGYPERVIDFAELNHLQRELRRAKEDATFTLPDSVDHPLWVRYEAPGGTNIHGYDLVQDTKRNLT | |
| | LILDKFILPDENGSWHEVKKVPFSLAKSKQFHRQVWLQEEQKQKKREVVFYDYSTNLPHLGTLAGAKL | |
| | QWDRNFLNKRTQQQIEETGEIGKVFFNISVDVRPAVEVKNGRLQNGLGKALTVLTHPDGTKIVTGWKA | |
| | EQLEKWVGESGRVSSLGLDSLSEGLRVMSIDLGQRTSATVSVFEITKEAPDNPYKFFYQLEGTEMFAV | |
| | HQRSFLLALPGENPPQKIKQMREIRWKERNRIKQQVDQLSAILRLHKKVNEDERIQAIDKLLQKVASW | |
| | QLNEEIATAWNQALSQLYSKAKENDLQWNQAIKNAHHQLEPVVGKQISLWRKDLSTGRQGIAGLSLWS | |
| | IEELEATKKLLTRWSKRSREPGVVKRIERFETFAKQIQHHINQVKENRLKQLANLIVMTALGYKYDQE | |
| | QKKWIEVYPACQVVLFENLRSYRFSFERSRRENKKLMEWSHRSIPKLVQMQGELFGLQVADVYAAYSS | |
| | RYHGRTGAPGIRCHALTEADLRNETNIIHELIEAGFIKEEHRPYLQQGDLVPWSGGELFATLQKPYDN | |
| | PRILTLHADINAAQNIQKRFWHPSMWFRVNCESVMEGEIVTYVPKNKTVHKKQGKTFRFVKVEGSDVY | |
| | EWAKWSKNRNKNTFSSITERKPPSSMILFRDPSGTFFKEQEWVEQKTFWGKVQSMIQAYMKKTIVQRM | |
| | EE | |
| DtCas12b | MVLGRKDDTAELRRALWTTHEHVNLAVAEVERVLLRCRGRSYWTLDRRGDPVHVPESQVAEDALAMAR | 60 |
| Dsulfonatronum | EAQRRNGWPVVGEDEEILLALRYLYEQIVPSCLLDDLGKPLKGDAQKIGTNYAGPLFDSDTCRRDEGK | |
| thiodismutans | DVACCGPFHEVAGKYLGALPEWATPISKQEFDGKDASHLRFKATGGDDAFFRVSIEKANAWYEDPANQ | |
| WP_031386437 | DALKNKAYNKDDWKKEKDKGISSWAVKYIQKQLQLGQDPRTEVRRKLWLELGLLPLFIPVFDKTMVGN | |
| | LWNRLAVRLALAHLLSWESWNHRAVQDQALARAKRDELAALFLGMEDGFAGLREYELRRNESIKQHAF | |
| | EPVDRPYVVSGRALRSWTRVREEWLRHGDTQESRKNICNRLQDRLRGKFGDPDVFHWLAEDGQEALWK | |
| | ERDCVTSFSLLNDADGLLEKRKGYALMTFADARLHPRWAMYEAPGGSNLRTYQIRKTENGLWADVVLL | |
| | SPRNESAAVEEKTFNVRLAPSGQLSNVSFDQIQKGSKMVGRCRYQSANQQFEGLLGGAEILFDRKRIA | |
| | NEQHGATDLASKPGHVWFKLTLDVRPQAPQGWLDGKGRPALPPEAKHFKTALSNKSKFADQVRPGLRV | |
| | LSVDLGVRSFAACSVFELVRGGPDQGTYFPAADGRTVDDPEKLWAKHERSFKITLPGENPSRKEEIAR | |
| | RAAMEELRSLNGDIRRLKAILRLSVLQEDDPRTEHLRLFMEAIVDDPAKSALNAELFKGFGDDRFRST | |
| | PDLWKQHCHFFHDKAEKVVAERFSRWRTETRPKSSSWQDWRERRGYAGGKSYWAVTYLEAVRGLILRW | |
| | NMRGRTYGEVNRQDKKQFGTVASALLHHINQLKEDRIKTGADMIIQAARGFVPRKNGAGWVQVHEPCR | |
| | LILFEDLARYRFRTDRSRRENSRLMRWSHREIVNEVGMQGELYGLHVDTTEAGFSSRYLASSGAPGVR | |
| | CRHLVEEDFHDGLPGMHLVGELDWLLPKDKDRTANEARRLLGGMVRPGMLVPWDGGELFATLNAASQL | |
| | HVIHADINAAQNLQRRFWGRCGEAIRIVCNQLSVDGSTRYEMAKAPKARLLGALQQLKNGDAPFHLTS | |
| | IPNSQKPENSYVMTPTNAGKKYRAGPGEKSSGEEDELALDIVEQAEELAQGRKTFFRDPSGVFFAPDR | |
| | WLPSEIYWSRIRRRIWQVTLERNSSGRQERAEMDEMPY | |

Cas9 with expanded PAM

| | | |
|---|---|---|
| Francisella | MSIYQEFVNKYSLSKTLRFELIPQGKTLENIKARGLILDDEKRAKDYKKAKQIIDKYHQFFIEEILSS | 61 |
| novicida Cpf1 | VCISEDLLQNYSDVYFKLKKSDDDNLQKDFKSAKDTIKKQISEYIKDSEKFKNLFNQNLIDAKKGQES | |
| | DLILWLKQSKDNGIELFKANSDITDIDEALEIIKSFKGWTTYFKGFHENRKNVYSSNDIPTSIIYRIV | |
| | DDNLPKFLENKAKYESLKDKAPEAINYEQIKKDLAEELTFDIDYKTSEVNQRVFSLDEVFEIANFNNY | |
| | LNQSGITKFNTIIGGKFVNGENTKRKGINEYINLYSQQINDKTLKKYKMSVLFKQILSDTESKSFVID | |
| | KLEDDSDVVTTMQSFYEQIAAFKTVEEKSIKETLSLLFDDLKAQKLDLSKIYFKNDKSLTDLSQQVFD | |
| | DYSVIGTAVLEYITQQIAPKNLDNPSKKEQELIAKKTEKAKYLSLETIKLALEEFNKHRDIDKQCRFE | |
| | EILANFAAIPMIFDEIAQNKDNLAQISIKYQNQGKKDLLQASAEDDVKAIKDLLDQTNNLLHKLKIFH | |
| | ISQSEDKANILDKDEHFYLVFEECYFELANIVPLYNKIRNYITQKPYSDEKFKLNFENSTLANGWDKN | |
| | KEPDNTAILFIKDDKYYLGVMNKKNNKIFDDKAIKENKGEGYKKIVYKLLPGANKMLPKVFFSAKSIK | |
| | FYNPSEDILRIRNHSTHTKNGSPQKGYEKFEFNIEDCRKFIDFYKQSISKHPEWKDFGFRFSDTQRYN | |
| | SIDEFYREVENQGYKLTFENISESYIDSVVNQGKLYLFQIYNKDFSAYSKGRPNLHTLYWKALFDERN | |
| | LQDVVYKLNGEAELFYRKQSIPKKITHPAKEAIANKNKDNPKKESVFEYDLIKDKRFTEDKFFFHCPI | |
| | TINFKSSGANKFNDEINLLLKEKANDVHILSIDRGERHLAYYTLVDGKGNIIKQDTFNIIGNDRMKTN | |
| | YHDKLAAIEKDRDSARKDWKKINNIKEMKEGYLSQVVHEIAKLVIEYNAIVVFEDLNFGFKRGRFKVE | |
| | KQVYQKLEKMLIEKLNYLVFKDNEFDKTGGVLRAYQLTAPFETFKKMGKQTGIIYYVPAGFTSKICPV | |
| | TGFVNQLYPKYESVSKSQEFFSKFDKICYNLDKGYFEFSFDYKNFGDKAAKGKWTIASFGSRLINFRN | |
| | SDKNHNWDTREVYPTKELEKLLKDYSIEYGHGECIKAAICGESDKKFFAKLTSVLNTILQMRNSKTGT | |
| | ELDYLISPVADVNGNFFDSRQAPKNMPQDADANGAYHIGLKGLMLLGRIKNNQEGKKLNLVIKNEEYF | |
| | EFVQNRNN | |
| Geobacillus | MKYKIGLDIGITSIGWAVINLDIPRIEDLGVRIFDRAENPKTGESLALPRRLARSARRRLRRRKHRLE | 62 |
| thermo | RIRRLFVREGILTKEELNKLFEKKHEIDVWQLRVEALDRKLNNDELARILLHLAKRRGFRSNRKSERT | |
| denitrificans | NKENSTMLKHIEENQSILSSYRTVAEMVVKDPKFSLHKRNKEDNYTNTVARDDLEREIKLIFAKQREY | |
| Cas9 | GNIVCTEAFEHEYISIWASQRPFASKDDIEKKVGFCTFEPKEKRAPKATYTFQSFTVWEHINKLRLVS | |
| | PGGIRALTDDERRLIYKQAFHKNKITFHDVRTLLNLPDDTRFKGLLYDRNTTLKENEKVRFLELGAYH | |
| | KIRKAIDSVYGKGAAKSFRPIDFDTFGYALTMFKDDTDIRSYLRNEYEQNGKRMENLADKVYDEELIE | |
| | ELLNLSFSKFGHLSLKALRNILPYMEQGEVYSTACERAGYTFTGPKKKQKTVLLPNIPPIANPVVMRA | |
| | LTQARKVVNAIIKKYGSPVSIHIELARELSQSFDERRKMQKEQEGNRKKNETAIRQLVEYGLTLNPTG | |
| | LDIVKFKLWSEQNGKCAYSLQPIEIERLLEPGYTEVDHVIPYSRSLDDSYTNKVLVLTKENREKGNRT | |
| | PAEYLGLGSERWQQFETFVLTNKQFSKKKRDRLLRLHYDENEENEFKNRNLNDTRYISRFLANFIREH | |
| | LKFADSDDKQKVYTVNGRITAHLRSRWNFNKNREESNLHHAVDAAIVACTTPSDIARVTAFYQRREQN | |
| | KELSKKTDPQFPQPWPHFADELQARLSKNPKESIKALNLGNYDNEKLESLQPVFVSRMPKRSITGAAH | |
| | QETLRRYIGIDERSGKIQTVVKKKLSEIQLDKTGHFPMYGKESDPRTYEAIRQRLLEHNNDPKKAFQE | |
| | PLYKPKKNGELGPIIRTIKIIDTTNQVIPLNDGKTVAYNSNIVRVDVFEKDGKYYCVPIYTIDMMKGI | |
| | LPNKAIEPNKPYSEWKEMTEDYTFRFSLYPNDLIRIEFPREKTIKTAVGEEIKIKDLFAYYQTIDSSN | |
| | GGLSLVSHDNNFSLRSIGSRTLKRFEKYQVDVLGNIYKVRGEKRVGVASSSHSKAGETIRPL | |
| Natrono- | MTVIDLDSTTTADELTSGHTYDISVTLTGVYDNTDEQHPRMSLAFEQDNGERRYITLWKNTTPKDVFT | 63 |
| bacterium | YDYATGSTYIFTNIDYEVKDGYENLTATYQTTVENATAQEVGTTDEDETFAGGEPLDHHLDDALNETP | |
| gregoryi | DDAETESDSGHVMTSFASRDQLPEWTLHTYTLTATDGAKTDTEYARRTLAYTVRQELYTDHDAAPVAT | |
| Argonaute | DGLMLLTPEPLGETPLDLDCGVRVEADETRTLDYTTAKDRLLARELVEEGLKRSLWDDYLVRGIDEVL | |
| | SKEPVLTCDEFDLHERYDLSVEVGHSGRAYLHINFRHRFVPKLTLADIDDDNIYPGLRVKTTYRPRRG | |
| | HIVWGLRDECATDSLNTLGNQSVVAYHRNNQTPINTDLLDAIEAADRRVVETRRQGHGDDAVSFPQEL | |
| | LAVEPNTHQIKQFASDGFHQQARSKTRLSASRCSEKAQAFAERLDPVRLNGSTVEFSSEFFTGNNEQQ | |
| | LRLLYENGESVLTFRDGARGAHPDETFSKGIVNPPESFEVAVVLPEQQADTCKAQWDTMADLLNQAGA | |
| | PPTRSETVQYDAFSSPESISLNVAGAIDPSEVDAAFVVLPPDQEGFADLASPTETYDELKKALANMGI | |
| | YSQMAYFDRFRDAKIFYTRNVALGLLAAAGGVAFTTEHAMPGDADMFIGIDVSRSYPEDGASGQINIA | |
| | ATATAVYKDGTILGHSSTRPQLGEKLQSTDVRDIMKNAILGYQQVTGESPTHIVIHRDGFMNEDLDPA | |
| | TEFLNEQGVEYDIVEIRKQPQTRLLAVSDVQYDTPVKSIAAINQNEPRATVATFGAPEYLATRDGGGL | |
| | PRPIQIERVAGETDIETLTRQVYLLSQSHIQVHNSTARLPITTAYADQASTHATKGYLVQTGAFESNV | |
| | GFL | |

Circular permutant Cas9s

| | | |
|---|---|---|
| CP1012 | DYKVYDVRKMIAKSEQEIGKATAKYFFYSNIMNFFKTEITLANGEIRKRPLIETNGETGEIVWDKGRD | 64 |
| | FATVRKVLSMPQVNIVKKTEVQTGGFSKESILPKRNSDKLIARKKDWDPKKYGGFDSPTVAYSVLVVA | |
| | KVEKGKSKKLKSVKELLGITIMERSSFEKNPIDFLEAKGYKEVKKDLIIKLPKYSLFELENGRKRMLA | |
| | SAGELQKGNELALPSKYVNFLYLASHYEKLKGSPEDNEQKQLFVEQHKHYLDEIIEQISEFSKRVILA | |
| | DANLDKVLSAYNKHRDKPIREQAENIIHLFTLTNLGAPAAFKYFDTTIDRKRYTSTKEVLDATLIHQS | |
| | ITGLYETRIDLSQLGGDGGSGGSGGSGGSGGSGGSGGDKKYSIGLAIGTNSVGWAVITDEYKVPSKKF | |
| | KVLGNTDRHSIKKNLIGALLFDSGETAEATRLKRTARRRYTRRKNRICYLQEIFSNEMAKVDDSFFHR | |
| | LEESFLVEEDKKHERHPIFGNIVDEVAYHEKYPTIYHLRKKLVDSTDKADLRLIYLALAHMIKFRGHF | |
| | LIEGDLNPDNSDVDKLFIQLVQTYNQLFEENPINASGVDAKAILSARLSKSRRLENLIAQLPGEKKNG | |
| | LFGNLIALSLGLTPNFKSNFDLAEDAKLQLSKDTYDDDLDNLLAQIGDQYADLFLAAKNLSDAILLSD | |
| | ILRVNTEITKAPLSASMIKRYDEHHQDLTLLKALVRQQLPEKYKEIFFDQSKNGYAGYIDGGASQEEF | |
| | YKFIKPILEKMDGTEELLVKLNREDLLRKQRTFDNGSIPHQIHLGELHAILRRQEDFYPFLKDNREKI | |
| | EKILTFRIPYYVGPLARGNSRFAWMTRKSEETITPWNFEEVVDKGASAQSFIERMTNFDKNLPNEKVL | |
| | PKHSLLYEYFTVYNELTKVKYVTEGMRKPAFLSGEQKKAIVDLLFKTNRKVTVKQLKEDYFKKIECFD | |
| | SVEISGVEDRFNASLGTYHDLLKIIKDKDFLDNEENEDILEDIVLTLTLFEDREMIEERLKTYAHLFD | |
| | DKVMKQLKRRRYTGWGRLSRKLINGIRDKQSGKTILDFLKSDGFANRNFMQLIHDDSLTFKEDIQKAQ | |
| | VSGQGDSLHEHIANLAGSPAIKKGILQTVKVVDELVKVMGRHKPENIVIEMARENQTTQKGQKNSRER | |
| | MKRIEEGIKELGSQILKEHPVENTQLQNEKLYLYYLQNGRDMYVDQELDINRLSDYDVDHIVPQSFLK | |
| | DDSIDNKVLTRSDKNRGKSDNVPSEEVVKKMKNYWRQLLNAKLITQRKFDNLTKAERGGLSELDKAGF | |
| | IKRQLVETRQITKHVAQILDSRMNTKYDENDKLIREVKVITLKSKLVSDFRKDFQFYKVREINNYHHA | |
| | HDAYLNAWGTALIKKYPKLESEFVYG | |
| CP1028 | EIGKATAKYFFYSNIMNFFKTEITLANGEIRKRPLIETNGETGEIVWDKGRDFATVRKVLSMPQVNIV | 65 |
| | KKTEVQTGGFSKESILPKRNSDKLIARKKDWDPKKYGGFDSPTVAYSVLVVAKVEKGKSKKLKSVKEL | |
| | LGITIMERSSFEKNPIDFLEAKGYKEVKKDLIIKLPKYSLFELENGRKRMLASAGELQKGNELALPSK | |
| | YVNFLYLASHYEKLKGSPEDNEQKQLFVEQHKHYLDEIIEQISEFSKRVILADANLDKVLSAYNKHRD | |
| | KPIREQAENIIHLFTLTNLGAPAAFKYFDTTIDRKRYTSTKEVLDATLIHQSITGLYETRIDLSQLGG | |
| | DGGSGGSGGSGGSGGSGGSGGMDKKYSIGLAIGTNSVGWAVITDEYKVPSKKFKVLGNTDRHSIKKNL | |
| | IGALLFDSGETAEATRLKRTARRRYTRRKNRICYLQEIFSNEMAKVDDSFFHRLEESFLVEEDKKHER | |
| | HPIFGNIVDEVAYHEKYPTIYHLRKKLVDSTDKADLRLIYLALAHMIKFRGHFLIEGDLNPDNSDVDK | |
| | LFIQLVQTYNQLFEENPINASGVDAKAILSARLSKSRRLENLIAQLPGEKKNGLFGNLIALSLGLTPN | |
| | FKSNFDLAEDAKLQLSKDTYDDDLDNLLAQIGDQYADLFLAAKNLSDAILLSDILRVNTEITKAPLSA | |
| | SMIKRYDEHHQDLTLLKALVRQQLPEKYKEIFFDQSKNGYAGYIDGGASQEEFYKFIKPILEKMDGTE | |
| | ELLVKLNREDLLRKQRTFDNGSIPHQIHLGELHAILRRQEDFYPFLKDNREKIEKILTFRIPYYVGPL | |
| | ARGNSRFAWMTRKSEETITPWNFEEVVDKGASAQSFIERMTNFDKNLPNEKVLPKHSLLYEYFTVYNE | |
| | LTKVKYVTEGMRKPAFLSGEQKKAIVDLLFKTNRKVTVKQLKEDYFKKIECFDSVEISGVEDRFNASL | |
| | GTYHDLLKIIKDKDFLDNEENEDILEDIVLTLTLFEDREMIEERLKTYAHLFDDKVMKQLKRRRYTGW | |
| | GRLSRKLINGIRDKQSGKTILDFLKSDGFANRNFMQLIHDDSLTFKEDIQKAQVSGQGDSLHEHIANL | |
| | AGSPAIKKGILQTVKVVDELVKVMGRHKPENIVIEMARENQTTQKGQKNSRERMKRIEEGIKELGSQI | |
| | LKEHPVENTQLQNEKLYLYYLQNGRDMYVDQELDINRLSDYDVDHIVPQSFLKDDSIDNKVLTRSDKN | |
| | RGKSDNVPSEEVVKKMKNYWRQLLNAKLITQRKFDNLTKAERGGLSELDKAGFIKRQLVETRQITKHV | |
| | AQILDSRMNTKYDENDKLIREVKVITLKSKLVSDFRKDFQFYKVREINNYHHAHDAYLNAVVGTALIK | |
| | KYPKLESEFVYGDYKVYDVRKMIAKSEQ | |
| CP1041 | NIMNFFKTEITLANGEIRKRPLIETNGETGEIVWDKGRDFATVRKVLSMPQVNIVKKTEVQTGGFSKE | 66 |
| | SILPKRNSDKLIARKKDWDPKKYGGFDSPTVAYSVLVVAKVEKGKSKKLKSVKELLGITIMERSSFEK | |
| | NPIDFLEAKGYKEVKKDLIIKLPKYSLFELENGRKRMLASAGELQKGNELALPSKYVNFLYLASHYEK | |
| | LKGSPEDNEQKQLFVEQHKHYLDEIIEQISEFSKRVILADANLDKVLSAYNKHRDKPIREQAENIIHL | |
| | FTLTNLGAPAAFKYFDTTIDRKRYTSTKEVLDATLIHQSITGLYETRIDLSQLGGDGGSGGSGGSGGS | |
| | GGSGGSGGDKKYSIGLAIGTNSVGWAVITDEYKVPSKKFKVLGNTDRHSIKKNLIGALLFDSGETAEA | |
| | TRLKRTARRRYTRRKNRICYLQEIFSNEMAKVDDSFFHRLEESFLVEEDKKHERHPIFGNIVDEVAYH | |
| | EKYPTIYHLRKKLVDSTDKADLRLIYLALAHMIKFRGHFLIEGDLNPDNSDVDKLFIQLVQTYNQLFE | |
| | ENPINASGVDAKAILSARLSKSRRLENLIAQLPGEKKNGLFGNLIALSLGLTPNFKSNFDLAEDAKLQ | |
| | LSKDTYDDDLDNLLAQIGDQYADLFLAAKNLSDAILLSDILRVNTEITKAPLSASMIKRYDEHHQDLT | |
| | LLKALVRQQLPEKYKEIFFDQSKNGYAGYIDGGASQEEFYKFIKPILEKMDGTEELLVKLNREDLLRK | |
| | QRTFDNGSIPHQIHLGELHAILRRQEDFYPFLKDNREKIEKILTFRIPYYVGPLARGNSRFAWMTRKS | |
| | EETITPWNFEEVVDKGASAQSFIERMTNFDKNLPNEKVLPKHSLLYEYFTVYNELTKVKYVTEGMRKP | |
| | AFLSGEQKKAIVDLLFKTNRKVTVKQLKEDYFKKIECFDSVEISGVEDRFNASLGTYHDLLKIIKDKD | |
| | FLDNEENEDILEDIVLTLTLFEDREMIEERLKTYAHLFDDKVMKQLKRRRYTGWGRLSRKLINGIRDK | |
| | QSGKTILDFLKSDGFANRNFMQLIHDDSLTFKEDIQKAQVSGQGDSLHEHIANLAGSPAIKKGILQTV | |
| | KVVDELVKVMGRHKPENIVIEMARENQTTQKGQKNSRERMKRIEEGIKELGSQILKEHPVENTQLQNE | |
| | KLYLYYLQNGRDMYVDQELDINRLSDYDVDHIVPQSFLKDDSIDNKVLTRSDKNRGKSDNVPSEEVVK | |
| | KMKNYWRQLLNAKLITQRKFDNLTKAERGGLSELDKAGFIKRQLVETRQITKHVAQILDSRMNTKYDE | |
| | NDKLIREVKVITLKSKLVSDFRKDFQFYKVREINNYHHAHDAYLNAVVGTALIKKYPKLESEFVYGDY | |
| | KVYDVRKMIAKSEQEIGKATAKYFFYS | |
| CP1249 | PEDNEQKQLFVEQHKHYLDEIIEQISEFSKRVILADANLDKVLSAYNKHRDKPIREQAENIIHLFTLT | 67 |
| | NLGAPAAFKYFDTTIDRKRYTSTKEVLDATLIHQSITGLYETRIDLSQLGGDGGSGGSGGSGGSGGSG | |
| | GSGGMDKKYSIGLAIGTNSVGWAVITDEYKVPSKKFKVLGNTDRHSIKKNLIGALLFDSGETAEATRL | |
| | KRTARRRYTRRKNRICYLQEIFSNEMAKVDDSFFHRLEESFLVEEDKKHERHPIFGNIVDEVAYHEKY | |
| | PTIYHLRKKLVDSTDKADLRLIYLALAHMIKFRGHFLIEGDLNPDNSDVDKLFIQLVQTYNQLFEENP | |
| | INASGVDAKAILSARLSKSRRLENLIAQLPGEKKNGLFGNLIALSLGLTPNFKSNFDLAEDAKLQLSK | |
| | DTYDDDLDNLLAQIGDQYADLFLAAKNLSDAILLSDILRVNTEITKAPLSASMIKRYDEHHQDLTLLK | |
| | ALVRQQLPEKYKEIFFDQSKNGYAGYIDGGASQEEFYKFIKPILEKMDGTEELLVKLNREDLLRKQRT | |
| | FDNGSIPHQIHLGELHAILRRQEDFYPFLKDNREKIEKILTFRIPYYVGPLARGNSRFAWMTRKSEET | |
| | ITPWNFEEVVDKGASAQSFIERMTNFDKNLPNEKVLPKHSLLYEYFTVYNELTKVKYVTEGMRKPAFL | |
| | SGEQKKAIVDLLFKTNRKVTVKQLKEDYFKKIECFDSVEISGVEDRFNASLGTYHDLLKIIKDKDFLD | |
| | NEENEDILEDIVLTLTLFEDREMIEERLKTYAHLFDDKVMKQLKRRRYTGWGRLSRKLINGIRDKQSG | |
| | KTILDFLKSDGFANRNFMQLIHDDSLTFKEDIQKAQVSGQGDSLHEHIANLAGSPAIKKGILQTVKVV | |
| | DELVKVMGRHKPENIVIEMARENQTTQKGQKNSRERMKRIEEGIKELGSQILKEHPVENTQLQNEKLY | |
| | LYYLQNGRDMYVDQELDINRLSDYDVDHIVPQSFLKDDSIDNKVLTRSDKNRGKSDNVPSEEVVKKMK | |
| | NYWRQLLNAKLITQRKFDNLTKAERGGLSELDKAGFIKRQLVETRQITKHVAQILDSRMNTKYDENDK | |
| | LIREVKVITLKSKLVSDFRKDFQFYKVREINNYHHAHDAYLNAVVGTALIKKYPKLESEFVYGDYKVY | |
| | DVRKMIAKSEQEIGKATAKYFFYSNIMNFFKTEITLANGEIRKRPLIETNGETGEIVWDKGRDFATVR | |
| | KVLSMPQVNIVKKTEVQTGGFSKESILPKRNSDKLIARKKDWDPKKYGGFDSPTVAYSVLVVAKVEKG | |
| | KSKKLKSVKELLGITIMERSSFEKNPIDFLEAKGYKEVKKDLIIKLPKYSLFELENGRKRMLASAGEL | |
| | QKGNELALPSKYVNFLYLASHYEKLKGS | |
| CP1300 | KPIREQAENIIHLFTLTNLGAPAAFKYFDTTIDRKRYTSTKEVLDATLIHQSITGLYETRIDLSQLGG | 68 |
| | DGGSGGSGGSGGSGGSGGSGGDKKYSIGLAIGTNSVGWAVITDEYKVPSKKFKVLGNTDRHSIKKNLI | |
| | GALLFDSGETAEATRLKRTARRRYTRRKNRICYLQEIFSNEMAKVDDSFFHRLEESFLVEEDKKHERH | |
| | PIFGNIVDEVAYHEKYPTIYHLRKKLVDSTDKADLRLIYLALAHMIKFRGHFLIEGDLNPDNSDVDKL | |
| | FIQLVQTYNQLFEENPINASGVDAKAILSARLSKSRRLENLIAQLPGEKKNGLFGNLIALSLGLTPNF | |
| | KSNFDLAEDAKLQLSKDTYDDDLDNLLAQIGDQYADLFLAAKNLSDAILLSDILRVNTEITKAPLSAS | |
| | MIKRYDEHHQDLTLLKALVRQQLPEKYKEIFFDQSKNGYAGYIDGGASQEEFYKFIKPILEKMDGTEE | |
| | LLVKLNREDLLRKQRTFDNGSIPHQIHLGELHAILRRQEDFYPFLKDNREKIEKILTFRIPYYVGPLA | |
| | RGNSRFAWMTRKSEETITPWNFEEVVDKGASAQSFIERMTNFDKNLPNEKVLPKHSLLYEYFTVYNEL | |
| | TKVKYVTEGMRKPAFLSGEQKKAIVDLLFKTNRKVTVKQLKEDYFKKIECFDSVEISGVEDRFNASLG | |
| | TYHDLLKIIKDKDFLDNEENEDILEDIVLTLTLFEDREMIEERLKTYAHLFDDKVMKQLKRRRYTGWG | |
| | RLSRKLINGIRDKQSGKTILDFLKSDGFANRNFMQLIHDDSLTFKEDIQKAQVSGQGDSLHEHIANLA | |
| | GSPAIKKGILQTVKVVDELVKVMGRHKPENIVIEMARENQTTQKGQKNSRERMKRIEEGIKELGSQIL | |
| | KEHPVENTQLQNEKLYLYYLQNGRDMYVDQELDINRLSDYDVDHIVPQSFLKDDSIDNKVLTRSDKNR | |
| | GKSDNVPSEEVVKKMKNYWRQLLNAKLITQRKFDNLTKAERGGLSELDKAGFIKRQLVETRQITKHVA | |
| | QILDSRMNTKYDENDKLIREVKVITLKSKLVSDFRKDFQFYKVREINNYHHAHDAYLNAVVGTALIKK | |
| | YPKLESEFVYGDYKVYDVRKMIAKSEQEIGKATAKYFFYSNIMNFFKTEITLANGEIRKRPLIETNGE | |
| | TGEIVWDKGRDFATVRKVLSMPQVNIVKKTEVQTGGFSKESILPKRNSDKLIARKKDWDPKKYGGFDS | |
| | PTVAYSVLVVAKVEKGKSKKLKSVKELLGITIMERSSFEKNPIDFLEAKGYKEVKKDLIIKLPKYSLF | |
| | ELENGRKRMLASAGELQKGNELALPSKYVNFLYLASHYEKLKGSPEDNEQKQLFVEQHKHYLDEIIEQ | |
| | ISEFSKRVILADANLDKVLSAYNKHRD | |
| CP1012 C- | DYKVYDVRKMIAKSEQEIGKATAKYFFYSNIMNFFKTEITLANGEIRKRPLIETNGETGEIVWDKGRD | 69 |
| terminal | FATVRKVLSMPQVNIVKKTEVQTGGFSKESILPKRNSDKLIARKKDWDPKKYGGFDSPTVAYSVLVVA | |
| fragment | KVEKGKSKKLKSVKELLGITIMERSSFEKNPIDFLEAKGYKEVKKDLIIKLPKYSLFELENGRKRMLA | |
| | SAGELQKGNELALPSKYVNFLYLASHYEKLKGSPEDNEQKQLFVEQHKHYLDEIIEQISEFSKRVILA | |
| | DANLDKVLSAYNKHRDKPIREQAENIIHLFTLTNLGAPAAFKYFDTTIDRKRYTSTKEVLDATLIHQS | |
| | ITGLYETRIDLSQLGGD | |
| CP1028 C- | EIGKATAKYFFYSNIMNFFKTEITLANGEIRKRPLIETNGETGEIVWDKGRDFATVRKVLSMPQVNIV | 70 |
| terminal | KKTEVQTGGFSKESILPKRNSDKLIARKKDWDPKKYGGFDSPTVAYSVLVVAKVEKGKSKKLKSVKEL | |
| fragment | LGITIMERSSFEKNPIDFLEAKGYKEVKKDLIIKLPKYSLFELENGRKRMLASAGELQKGNELALPSK | |
| | YVNFLYLASHYEKLKGSPEDNEQKQLFVEQHKHYLDEIIEQISEFSKRVILADANLDKVLSAYNKHRD | |
| | KPIREQAENIIHLFTLTNLGAPAAFKYFDTTIDRKRYTSTKEVLDATLIHQSITGLYETRIDLSQLGG | |
| | D | |
| CP1041 C- | NIMNFFKTEITLANGEIRKRPLIETNGETGEIVWDKGRDFATVRKVLSMPQVNIVKKTEVQTGGFSKE | 71 |
| terminal | SILPKRNSDKLIARKKDWDPKKYGGFDSPTVAYSVLVVAKVEKGKSKKLKSVKELLGITIMERSSFEK | |
| fragment | NPIDFLEAKGYKEVKKDLIIKLPKYSLFELENGRKRMLASAGELQKGNELALPSKYVNFLYLASHYEK | |
| | LKGSPEDNEQKQLFVEQHKHYLDEIIEQISEFSKRVILADANLDKVLSAYNKHRDKPIREQAENIIHL | |
| | FTLTNLGAPAAFKYFDTTIDRKRYTSTKEVLDATLIHQSITGLYETRIDLSQLGGD | |
| CP1249 C- | PEDNEQKQLFVEQHKHYLDEIIEQISEFSKRVILADANLDKVLSAYNKHRDKPIREQAENIIHLFTLT | 72 |
| terminal | NLGAPAAFKYFDTTIDRKRYTSTKEVLDATLIHQSITGLYETRIDLSQLGGD | |
| fragment | | |
| CP1300 C- | KPIREQAENIIHLFTLTNLGAPAAFKYFDTTIDRKRYTSTKEVLDATLIHQSITGLYETRIDLSQLGG | 73 |
| terminal | D | |
| fragment | | |

Cas9 with modified PAM

| | | |
|---|---|---|
| SpCas9-VRQR | DKKYSIGLDIGTNSVGWAVITDEYKVPSKKFKVLGNTDRHSIKKNLIGALLFDSGETAEATRLKRTAR | 74 |
| | RRYTRRKNRICYLQEIFSNEMAKVDDSFFHRLEESFLVEEDKKHERHPIFGNIVDEVAYHEKYPTIYH | |
| | LRKKLVDSTDKADLRLIYLALAHMIKFRGHFLIEGDLNPDNSDVDKLFIQLVQTYNQLFEENPINASG | |
| | VDAKAILSARLSKSRRLENLIAQLPGEKKNGLFGNLIALSLGLTPNFKSNFDLAEDAKLQLSKDTYDD | |
| | DLDNLLAQIGDQYADLFLAAKNLSDAILLSDILRVNTEITKAPLSASMIKRYDEHHQDLTLLKALVRQ | |
| | QLPEKYKEIFFDQSKNGYAGYIDGGASQEEFYKFIKPILEKMDGTEELLVKLNREDLLRKQRTFDNGS | |
| | IPHQIHLGELHAILRRQEDFYPFLKDNREKIEKILTFRIPYYVGPLARGNSRFAWMTRKSEETITPWN | |
| | FEEVVDKGASAQSFIERMTNFDKNLPNEKVLPKHSLLYEYFTVYNELTKVKYVTEGMRKPAFLSGEQK | |
| | KAIVDLLFKTNRKVTVKQLKEDYFKKIECFDSVEISGVEDRFNASLGTYHDLLKIIKDKDFLDNEENE | |
| | DILEDIVLTLTLFEDREMIEERLKTYAHLFDDKVMKQLKRRRYTGWGRLSRKLINGIRDKQSGKTILD | |
| | FLKSDGFANRNFMQLIHDDSLTFKEDIQKAQVSGQGDSLHEHIANLAGSPAIKKGILQTVKVVDELVK | |
| | VMGRHKPENIVIEMARENQTTQKGQKNSRERMKRIEEGIKELGSQILKEHPVENTQLQNEKLYLYYLQ | |
| | NGRDMYVDQELDINRLSDYDVDAIVPQSFLKDDSIDNKVLTRSDKNRGKSDNVPSEEVVKKMKNYWRQ | |
| | LLNAKLITQRKFDNLTKAERGGLSELDKAGFIKRQLVETRQITKHVAQILDSRMNTKYDENDKLIREV | |
| | KVITLKSKLVSDFRKDFQFYKVREINNYHHAHDAYLNAVVGTALIKKYPKLESEFVYGDYKVYDVRKM | |
| | IAKSEQEIGKATAKYFFYSNIMNFFKTEITLANGEIRKRPLIETNGETGEIVWDKGRDFATVRKVLSM | |
| | PQVNIVKKTEVQTGGFSKESILPKRNSDKLIARKKDWDPKKYGGFVSPTVAYSVLVVAKVEKGKSKKL | |
| | KSVKELLGITIMERSSFEKNPIDFLEAKGYKEVKKDLIIKLPKYSLFELENGRKRMLASARELQKGNE | |
| | LALPSKYVNFLYLASHYEKLKGSPEDNEQKQLFVEQHKHYLDEIIEQISEFSKRVILADANLDKVLSA | |
| | YNKHRDKPIREQAENIIHLFTLTNLGAPAAFKYFDTTIDRKQYRSTKEVLDATLIHQSITGLYETRID | |
| | LSQLGGD | |
| SpCas9-VQR | DKKYSIGLDIGTNSVGWAVITDEYKVPSKKFKVLGNTDRHSIKKNLIGALLFDSGETAEATRLKRTAR | 75 |
| | RRYTRRKNRICYLQEIFSNEMAKVDDSFFHRLEESFLVEEDKKHERHPIFGNIVDEVAYHEKYPTIYH | |
| | LRKKLVDSTDKADLRLIYLALAHMIKFRGHFLIEGDLNPDNSDVDKLFIQLVQTYNQLFEENPINASG | |
| | VDAKAILSARLSKSRRLENLIAQLPGEKKNGLFGNLIALSLGLTPNFKSNFDLAEDAKLQLSKDTYDD | |
| | DLDNLLAQIGDQYADLFLAAKNLSDAILLSDILRVNTEITKAPLSASMIKRYDEHHQDLTLLKALVRQ | |
| | QLPEKYKEIFFDQSKNGYAGYIDGGASQEEFYKFIKPILEKMDGTEELLVKLNREDLLRKQRTFDNGS | |
| | IPHQIHLGELHAILRRQEDFYPFLKDNREKIEKILTFRIPYYVGPLARGNSRFAWMTRKSEETITPWN | |
| | FEEVVDKGASAQSFIERMTNFDKNLPNEKVLPKHSLLYEYFTVYNELTKVKYVTEGMRKPAFLSGEQK | |
| | KAIVDLLFKTNRKVTVKQLKEDYFKKIECFDSVEISGVEDRFNASLGTYHDLLKIIKDKDFLDNEENE | |
| | DILEDIVLTLTLFEDREMIEERLKTYAHLFDDKVMKQLKRRRYTGWGRLSRKLINGIRDKQSGKTILD | |
| | FLKSDGFANRNFMQLIHDDSLTFKEDIQKAQVSGQGDSLHEHIANLAGSPAIKKGILQTVKVVDELVK | |
| | VMGRHKPENIVIEMARENQTTQKGQKNSRERMKRIEEGIKELGSQILKEHPVENTQLQNEKLYLYYLQ | |
| | NGRDMYVDQELDINRLSDYDVDAIVPQSFLKDDSIDNKVLTRSDKNRGKSDNVPSEEVVKKMKNYWRQ | |
| | LLNAKLITQRKFDNLTKAERGGLSELDKAGFIKRQLVETRQITKHVAQILDSRMNTKYDENDKLIREV | |
| | KVITLKSKLVSDFRKDFQFYKVREINNYHHAHDAYLNAVVGTALIKKYPKLESEFVYGDYKVYDVRKM | |
| | IAKSEQEIGKATAKYFFYSNIMNFFKTEITLANGEIRKRPLIETNGETGEIVWDKGRDFATVRKVLSM | |
| | PQVNIVKKTEVQTGGFSKESILPKRNSDKLIARKKDWDPKKYGGFVSPTVAYSVLVVAKVEKGKSKKL | |
| | KSVKELLGITIMERSSFEKNPIDFLEAKGYKEVKKDLIIKLPKYSLFELENGRKRMLASAGELQKGNE | |
| | LALPSKYVNFLYLASHYEKLKGSPEDNEQKQLFVEQHKHYLDEIIEQISEFSKRVILADANLDKVLSA | |
| | YNKHRDKPIREQAENIIHLFTLTNLGAPAAFKYFDTTIDRKQYRSTKEVLDATLIHQSITGLYETRID | |
| | LSQLGGD | |
| SpCas9-VRER | DKKYSIGLDIGTNSVGWAVITDEYKVPSKKFKVLGNTDRHSIKKNLIGALLFDSGETAEATRLKRTAR | 76 |
| | RRYTRRKNRICYLQEIFSNEMAKVDDSFFHRLEESFLVEEDKKHERHPIFGNIVDEVAYHEKYPTIYH | |
| | LRKKLVDSTDKADLRLIYLALAHMIKFRGHFLIEGDLNPDNSDVDKLFIQLVQTYNQLFEENPINASG | |
| | VDAKAILSARLSKSRRLENLIAQLPGEKKNGLFGNLIALSLGLTPNFKSNFDLAEDAKLQLSKDTYDD | |
| | DLDNLLAQIGDQYADLFLAAKNLSDAILLSDILRVNTEITKAPLSASMIKRYDEHHQDLTLLKALVRQ | |
| | QLPEKYKEIFFDQSKNGYAGYIDGGASQEEFYKFIKPILEKMDGTEELLVKLNREDLLRKQRTFDNGS | |
| | IPHQIHLGELHAILRRQEDFYPFLKDNREKIEKILTFRIPYYVGPLARGNSRFAWMTRKSEETITPWN | |
| | FEEVVDKGASAQSFIERMTNFDKNLPNEKVLPKHSLLYEYFTVYNELTKVKYVTEGMRKPAFLSGEQK | |
| | KAIVDLLFKTNRKVTVKQLKEDYFKKIECFDSVEISGVEDRFNASLGTYHDLLKIIKDKDFLDNEENE | |
| | DILEDIVLTLTLFEDREMIEERLKTYAHLFDDKVMKQLKRRRYTGWGRLSRKLINGIRDKQSGKTILD | |
| | FLKSDGFANRNFMQLIHDDSLTFKEDIQKAQVSGQGDSLHEHIANLAGSPAIKKGILQTVKVVDELVK | |
| | VMGRHKPENIVIEMARENQTTQKGQKNSRERMKRIEEGIKELGSQILKEHPVENTQLQNEKLYLYYLQ | |
| | NGRDMYVDQELDINRLSDYDVDAIVPQSFLKDDSIDNKVLTRSDKNRGKSDNVPSEEVVKKMKNYWRQ | |
| | LLNAKLITQRKFDNLTKAERGGLSELDKAGFIKRQLVETRQITKHVAQILDSRMNTKYDENDKLIREV | |
| | KVITLKSKLVSDFRKDFQFYKVREINNYHHAHDAYLNAVVGTALIKKYPKLESEFVYGDYKVYDVRKM | |
| | IAKSEQEIGKATAKYFFYSNIMNFFKTEITLANGEIRKRPLIETNGETGEIVWDKGRDFATVRKVLSM | |
| | PQVNIVKKTEVQTGGFSKESILPKRNSDKLIARKKDWDPKKYGGFVSPTVAYSVLVVAKVEKGKSKKL | |
| | KSVKELLGITIMERSSFEKNPIDFLEAKGYKEVKKDLIIKLPKYSLFELENGRKRMLASARELQKGNE | |
| | LALPSKYVNFLYLASHYEKLKGSPEDNEQKQLFVEQHKHYLDEIIEQISEFSKRVILADANLDKVLSA | |
| | YNKHRDKPIREQAENIIHLFTLTNLGAPAAFKYFDTTIDRKEYRSTKEVLDATLIHQSITGLYETRID | |
| | LSQLGGD | |
| SpCas9-NG | MDKKYSIGLDIGTNSVGWAVITDEYKVPSKKFKVLGNTDRHSIKKNLIGALLFDSGETAEATRLKRTA | 77 |
| | RRRYTRRKNRICYLQEIFSNEMAKVDDSFFHRLEESFLVEEDKKHERHPIFGNIVDEVAYHEKYPTIY | |
| | HLRKKLVDSTDKADLRLIYLALAHMIKFRGHFLIEGDLNPDNSDVDKLFIQLVQTYNQLFEENPINAS | |
| | GVDAKAILSARLSKSRRLENLIAQLPGEKKNGLFGNLIALSLGLTPNFKSNFDLAEDAKLQLSKDTYD | |
| | DDLDNLLAQIGDQYADLFLAAKNLSDAILLSDILRVNTEITKAPLSASMIKRYDEHHQDLTLLKALVR | |
| | QQLPEKYKEIFFDQSKNGYAGYIDGGASQEEFYKFIKPILEKMDGTEELLVKLNREDLLRKQRTFDNG | |
| | SIPHQIHLGELHAILRRQEDFYPFLKDNREKIEKILTFRIPYYVGPLARGNSRFAWMTRKSEETITPW | |
| | NFEEVVDKGASAQSFIERMTNFDKNLPNEKVLPKHSLLYEYFTVYNELTKVKYVTEGMRKPAFLSGEQ | |
| | KKAIVDLLFKTNRKVTVKQLKEDYFKKIECFDSVEISGVEDRFNASLGTYHDLLKIIKDKDFLDNEEN | |
| | EDILEDIVLTLTLFEDREMIEERLKTYAHLFDDKVMKQLKRRRYTGWGRLSRKLINGIRDKQSGKTIL | |
| | DFLKSDGFANRNFMQLIHDDSLTFKEDIQKAQVSGQGDSLHEHIANLAGSPAIKKGILQTVKVVDELV | |
| | KVMGRHKPENIVIEMARENQTTQKGQKNSRERMKRIEEGIKELGSQILKEHPVENTQLQNEKLYLYYL | |
| | QNGRDMYVDQELDINRLSDYDVDHIVPQSFLKDDSIDNKVLTRSDKNRGKSDNVPSEEVVKKMKNYWR | |
| | QLLNAKLITQRKFDNLTKAERGGLSELDKAGFIKRQLVETRQITKHVAQILDSRMNTKYDENDKLIRE | |
| | VKVITLKSKLVSDFRKDFQFYKVREINNYHHAHDAYLNAVVGTALIKKYPKLESEFVYGDYKVYDVRK | |
| | MIAKSEQEIGKATAKYFFYSNIMNFFKTEITLANGEIRKRPLIETNGETGEIVWDKGRDFATVRKVLS | |
| | MPQVNIVKKTEVQTGGFSKESIRPKRNSDKLIARKKDWDPKKYGGFVSPTVAYSVLVVAKVEKGKSKK | |
| | LKSVKELLGITIMERSSFEKNPIDFLEAKGYKEVKKDLIIKLPKYSLFELENGRKRMLASARFLQKGN | |
| | ELALPSKYVNFLYLASHYEKLKGSPEDNEQKQLFVEQHKHYLDEIIEQISEFSKRVILADANLDKVLS | |
| | AYNKHRDKPIREQAENIIHLFTLTNLGAPRAFKYFDTTIDRKVYRSTKEVLDATLIHQSITGLYETRI | |
| | DLSQLGGD | |

Adenine deaminases

| | | SEQ ID |
|---|---|---|
| DESCRIPTION | SEQUENCE | NO: |
| E. coli TadA | MSEVEFSHEYWMRHALTLAKRAWDEREVPVGAVLVHNNRVIGEGWNRPIGRHDPTAHAEIMALRQGGL | 78 |
| | VMQNYRLIDATLYVTLEPCVMCAGAMIHSRIGRVVFGARDAKTGAAGSLMDVLHHPGMNHRVEITEGI | |
| | LADECAALLSDFFRMRRQEIKAQKKAQSSTD | |
| E. coli TadA | MSEVEFSHEYWMRHALTLAKRARDEREVPVGAVLVLNNRVIGEGWNRAIGLHDPTAHAEIMALRQGGL | 79 |
| 7.10 | VMQNYRLIDATLYVTFEPCVMCAGAMIHSRIGRVVFGVRNAKTGAAGSLMDVLHYPGMNHRVEITEGI | |
| | LADECAALLCYFFRMPRQVFNAQKKAQSSTD | |
| E. coli TadA | MSEVEFSHEYWMRHALTLAKRARDEREVPVGAVLVLNNRVIGEGWNRAIGLHDPTAHAEIMALRQGGL | 80 |
| 7.10 (V106W) | VMQNYRLIDATLYVTFEPCVMCAGAMIHSRIGRVVFGWRNAKTGAAGSLMDVLHYPGMNHRVEITEGI | |
| | LADECAALLCYFFRMPRQVFNAQKKAQSSTD | |
| Staphylococcus | MGSHMTNDIYFMTLAIEEAKKAAQLGEVPIGAIITKDDEVIARAHNLRETLQQPTAHAEHIAIERAAK | 81 |
| aureus TadA | VLGSWRLEGCTLYVTLEPCVMCAGTIVMSRIPRVVYGADDPKGGCSGSLMNLLQQSNFNHRAIVDKGV | |
| | LKEACSTLLTTFFKNLRANKKSTN | |
| Bacillus | MTQDELYMKEAIKEAKKAEEKGEVPIGAVLVINGEIIARAHNLRETEQRSIAHAEMLVIDEACKALGT | 82 |
| subtilis TadA | WRLEGATLYVTLEPCPMCAGAVVLSRVEKVVFGAFDPKGGCSGTLMNLLQEERFNHQAEVVSGVLEEE | |
| | CGGMLSAFFRELRKKKKAARKNLSE | |
| Salmonella | MPPAFITGVTSLSDVELDHEYWMRHALTLAKRAWDEREVPVGAVLVHNHRVIGEGWNRPIGRHDPTAH | 83 |
| typhimurium | AEIMALRQGGLVLQNYRLLDTTLYVTLEPCVMCAGAMVHSRIGRVVFGARDAKTGAAGSLIDVLHHPG | |
| TadA | MNHRVEIIEGVLRDECATLLSDFFRMRRQEIKALKKADRAEGAGPAV | |
| Shewanella | MDEYWMQVAMQMAEKAEAAGEVPVGAVLVKDGQQIATGYNLSISQHDPTAHAEILCLRSAGKKLENYR | 84 |
| putrefaciens | LLDATLYITLEPCAMCAGAMVHSRIARVVYGARDEKTGAAGTVVNLLQHPAFNHQVEVTSGVLAEACS | |
| TadA | AQLSRFFKRRRDEKKALKLAQRAQQGIE | |
| Haemophilus | MDAAKVRSEFDEKMMRYALELADKAEALGEIPVGAVLVDDARNIIGEGWNLSIVQSDPTAHAEIIALR | 85 |
| influenzae | NGAKNIQNYRLLNSTLYVTLEPCTMCAGAILHSRIKRLVFGASDYKTGAIGSRFHFFDDYKMNHTLEI | |
| F3031 TadA | TSGVLAEECSQKLSTFFQKRREEKKIEKALLKSLSDK | |
| Caulobacter | MRTDESEDQDHRMMRLALDAARAAAEAGETPVGAVILDPSTGEVIATAGNGPIAAHDPTAHAEIAAMR | 86 |
| crescentus | AAAAKLGNYRLTDLTLWTLEPCAMCAGAISHARIGRWFGADDPKGGAWHGPKFFAQPTCHWRPEV | |
| TadA | TGGVLADESADLLRGFFRARRKAK1 | |
| Geobacter | MSSLKKTPIRDDAYWMGKAIREAAKAAARDEVPIGAVIVRDGAVIGRGHNLREGSNDPSAHAEMIAIR | 87 |
| sulfurreducens | QAARRSANWRLTGATLYVTLEPCLMCMGAIILARLERVVFGCYDPKGAAGSLYDLSADPRLNHQVRLS | |
| TadA | PGVCQEECGTMLSDFFRDLRRRKKAKATPALFIDERKVPPEP | |
| E. coli TadA | MSEVEFSHEYWMRHALTLAKRAWDEREVPVGAVLVHNNRVIGEGWNRPIGRHDPTAHAEIMALRQGGL | 78 |
| | VMQNYRLIDATLYVTLEPCVMCAGAMIHSRIGRVVFGARDAKTGAAGSLMDVLHHPGMNHRVEITEGI | |
| | LADECAALLSDFFRMRRQEIKAQKKAQSSTD | |
| ecTadA | MRRAFITGVFFLSEVEFSHEYWMRHALTLAKRAWDEREVPVGAVLVHNNRVIGEGWNRPIGRHDPTAH | 89 |
| | AEIMALRQGGLVMQNYRLIDATLYVTLEPCVMCAGAMIHSRIGRVVFGARDAKTGAAGSLMDVLHHPG | |
| | MNHRVEITEGILADECAALLSDFFRMRRQEIKAQKKAQSSTD | |
| ABES TadA* | TCTGAGGTGGAGTTTTCCCACGAGTACTGGATGAGACATGCCCTGACCCTGGCCAAGAGGGCACGGGA | 90 |
| monomer (aka | TGAGAGGGAGGTGCCTGTGGGAGCCGTGCTGGTGCTGAACAATAGAGTGATCGGCGAGGGCTGGAACA | |
| TadA-8e) | GAGCCATCGGCCTGCACGACCCAACAGCCCATGCCGAAATTATGGCCCTGAGACAGGGCGGCCTGGTC | |
| | ATGCAGAACTACAGACTGATTGACGCCACCCTGTACGTGACATTCGAGCCTTGCGTGATGTGCGCCGG | |
| | CGCCATGATCCACTCTAGGATCGGCCGCGTGGTGTTTGGCGTGAGGAACTCAAAAAGAGGCGCCGCAG | |
| | GCTCCCTGATGAACGTGCTGAACTACCCCGGCATGAATCACCGCGTCGAAATTACCGAGGGAATCCTG | |
| | GCAGATGAATGTGCCGCCCTGCTGTGCGATTTCTATCGGATGCCTAGACAGGTGTTCAATGCTCAGAA | |
| | GAAGGCCCAGAGCTCCATCAAC | |
| ABES TadA* | MSEVEFSHEYWMRHALTLAKRARDEREVPVGAVLVLNNRVIGEGWNRAIGLHDPTAHAEIMALRQGGL | 91 |
| monomer (aka | VMQNYRLIDATLYVTFEPCVMCAGAMIHSRIGRVVFGVRNSKRGAAGSLMNVLNYPGMNHRVEITEGI | |
| TadA-8e) | LADECAALLCDFYRMPRQVFNAQKKAQSSIN | |
| ABES TadA* | MSEVEFSHEYWMRHALTLAKRARDEREVPVGAVLVLNNRVIGEGWNRAIGLHDPTAHAEIMALRQGGL | 462 |
| V106W variant | VMQNYRLIDATLYVTFEPCVMCAGAMIHSRIGRVVFGWRNSKRGAAGSLMNVLNYPGMNHRVEITEGI | |
| | LADECAALLCDFYRMPRQVFNAQKKAQSSIN | |
| E. coli TadA* | MSEVEFSHEYWMRHALTLAKRARDEREVPVGAVLVLNNRVIGEGWNRAIGLHDPTAHAEIMALRQGGL | 79 |
| 7.10 | VMQNYRLIDATLYVTFEPCVMCAGAMIHSRIGRVVFGVRNAKTGAAGSLMDVLHYPGMNHRVEITEGI | |
| | LADECAALLCYFFRMPRQVFNAQKKAQSSTD | |
| ABE7.10 TadA* | TCTGAGGTGGAGTTTTCCCACGAGTACTGGATGAGACATGCCCTGACCCTGGCCAAGAGGGCACGCGA | 404 |
| monomer | TGAGAGGGAGGTGCCTGTGGGAGCCGTGCTGGTGCTGAACAATAGAGTGATCGGCGAGGGCTGGAACA | |
| | GAGCCATCGGCCTGCACGACCCAACAGCCCATGCCGAAATTATGGCCCTGAGACAGGGCGGCCTGGTC | |
| | ATGCAGAACTACAGACTGATTGACGCCACCCTGTACGTGACATTCGAGCCTTGCGTGATGTGCGCCGG | |
| | CGCCATGATCCACTCTAGGATCGGCCGCGTGGTGTTTGGCGTGAGGAACGCAAAAACCGGCGCCGCAG | |
| | GCTCCCTGATGGACGTGCTGCACTACCCCGGCATGAATCACCGCGTCGAAATTACCGAGGGAATCCTG | |
| | GCAGATGAATGTGCCGCCCTGCTGTGCTATTTCTTTCGGATGCCTAGACAGGTGTTCAATGCTCAGAA | |
| | GAAGGCCCAGAGCTCCACCGAC | |

Cytidine deaminases

| | | SEQ ID |
|---|---|---|
| DESCRIPTION | SEQUENCE | NO: |
| Human AID | MDSLLMNRRKFLYQFKNVRWAKGRRETYLCYVVKRRDSATSFSLDFGYLRNKNGCHVELLFLRYISDW | 92 |
| | DLDPGRCYRVTWFTSWSPCYDCARHVADFLRGNPNLSLRIFTARLYFCEDRKAEPEGLRRLHRAGVQI | |
| | AIMTFKDYFYCWNTFVENHERTFKAWEGLHENSVRLSRQLRRILLPLYEVDDLRDAFRTLGL | |
| Mouse AID | MDSLLMKQKKFLYHFKNVRWAKGRHETYLCYVVKRRDSATSCSLDFGHLRNKSGCHVELLFLRYISDW | 93 |
| | DLDPGRCYRVTWFTSWSPCYDCARHVAEFLRWNPNLSLRIFTARLYFCEDRKAEPEGLRRLHRAGVQI | |
| | GIMTFKDYFYCWNTFVENRERTFKAWEGLHENSVRLTRQLRRILLPLYEVDDLRDAFRMLGF | |
| Dog AID | MDSLLMKQRKFLYHFKNVRWAKGRHETYLCYVVKRRDSATSFSLDFGHLRNKSGCHVELLFLRYISDW | 94 |
| | DLDPGRCYRVTWFTSWSPCYDCARHVADFLRGYPNLSLRIFAARLYFCEDRKAEPEGLRRLHRAGVQI | |
| | AIMTFKDYFYCWNTFVENREKTFKAWEGLHENSVRLSRQLRRILLPLYEVDDLRDAFRTLGL | |
| Bovine AID | MDSLLKKQRQFLYQFKNVRWAKGRHETYLCYVVKRRDSPTSFSLDFGHLRNKAGCHVELLFLRYISDW | 95 |
| | DLDPGRCYRVTWFTSWSPCYDCARHVADFLRGYPNLSLRIFTARLYFCDKERKAEPEGLRRLHRAGVQ | |
| | IAIMTFKDYFYCWNTFVENHERTFKAWEGLHENSVRLSRQLRRILLPLYEVDDLRDAFRTLGL | |
| Rat: AID | MAVGSKPKAALVGPHWERERIWCFLCSTGLGTQQTGQTSRWLRPAATQDPVSPPRSLLMKQRKFLYHF | 96 |
| | KNVRWAKGRHETYLCYVVKRRDSATSFSLDFGYLRNKSGCHVELLFLRYISDWDLDPGRCYRVTWFTS | |
| | WSPCYDCARHVADFLRGNPNLSLRIFTARLTGWGALPAGLMSPARPSDYFYCWNTFVENHERTFKAWE | |
| | GLHENSVRLSRRLRRILLPLYEVDDLRDAFRTLGL | |
| Mouse APOBEC-3 | MGPFCLGCSHRKCYSPIRNLISQETFKFHFKNLGYAKGRKDTFLCYEVTRKDCDSPVSLHHGVFKNKD | 97 |
| | NIHAEICFLYWFHDKVLKVLSPREEFKITWYMSWSPCFECAEQIVRFLATHHNLSLDIFSSRLYNVQD | |
| | PETQQNLCRLVQEGAQVAAMDLYEFKKCWKKFVDNGGRRFRPWKRLLTNFRYQDSKLQEILRPCYIPV | |
| | PSSSSSTLSNICLTKGLPETRFCVEGRRMDPLSEEEFYSQFYNQRVKHLCYYHRMKPYLCYQLEQFNG | |
| | QAPLKGCLLSEKGKQHAEILFLDKIRSMELSQVTITCYLTWSPCPNCAWQLAAFKRDRPDLILHIYTS | |
| | RLYFHWKRPFQKGLCSLWQSGILVDVMDLPQFTDCWTNFVNPKRPFWPWKGLEIISRRTQRRLRRIKE | |
| | SWGLQDLVNDFGNLQLGPPMS | |
| Rat APOBEC-3 | MGPFCLGCSHRKCYSPIRNLISQETFKFHFKNLRYAIDRKDTFLCYEVTRKDCDSPVSLHHGVFKNKD | 98 |
| | NIHAEICFLYWFHDKVLKVLSPREEFKITWYMSWSPCFECAEQVLRFLATHHNLSLDIFSSRLYNIRD | |
| | PENQQNLCRLVQEGAQVAAMDLYEFKKCWKKFVDNGGRRFRPWKKLLTNFRYQDSKLQEILRPCYIPV | |
| | PSSSSSTLSNICLTKGLPETRFCVERRRVHLLSEEEFYSQFYNQRVKHLCYYHGVKPYLCYQLEQFNG | |
| | QAPLKGCLLSEKGKQHAEILFLDKIRSMELSQVIITCYLTWSPCPNCAWQLAAFKRDRPDLILHIYTS | |
| | RLYFHWKRPFQKGLCSLWQSGILVDVMDLPQFTDCWTNFVNPKRPFWPWKGLEIISRRTQRRLHRIKE | |
| | SWGLQDLVNDFGNLQLGPPMS | |
| Rhesus macaque | MVEPMDPRTFVSNFNNRPILSGLNTVWLCCEVKTKDPSGPPLDAKIFQGKVYSKAKYHPEMRFLRWFH | 99 |
| APOBEC-3G | KWRQLHHDQEYKVTWYVSWSPCTRCANSVATFLAKDPKVTLTIFVARLYYFWKPDYQQALRILCQKRG | |
| | GPHATMKIMNYNEFQDCWNKFVDGRGKPFKPRNNLPKHYTLLQATLGELLRHLMDPGTFTSNFNNKPW | |
| | VSGQHETYLCYKVERLHNDTWVPLNQHRGFLRNQAPNIHGFPKGRHAELCFLDLIPFWKLDGQQYRVT | |
| | CFTSWSPCFSCAQEMAKFISNNEHVSLCIFAARIYDDQGRYQEGLRALHRDGAKIAMMNYSEFEYCWD | |
| | TFVDRQGRPFQPWDGLDEHSQALSGRLRAI | |
| Chimpanzee | MKPHFRNPVERMYQDTFSDNFYNRPILSHRNTVWLCYEVKTKGPSRPPLDAKIFRGQVYSKLKYHPEM | 100 |
| APOBEC-3G | RFFHWFSKWRKLHRDQEYEVTWYISWSPCTKCTRDVATFLAEDPKVTLTIFVARLYYFWDPDYQEALR | |
| | SLCQKRDGPRATMKIMNYDEFQHCWSKFVYSQRELFEPWNNLPKYYILLHIMLGEILRHSMDPPTFTS | |
| | NFNNELWVRGRHETYLCYEVERLHNDTWVLLNQRRGFLCNQAPHKHGFLEGRHAELCFLDVIPFWKLD | |
| | LHQDYRVTCFTSWSPCFSCAQEMAKFISNNKHVSLCIFAARIYDDQGRCQEGLRTLAKAGAKISIMTY | |
| | SEFKHCWDTFVDHQGCPFQPWDGLEEHSQALSGRLRAILQNQGN | |
| Green monkey | MNPQIRNMVEQMEPDIFVYYFNNRPILSGRNTVWLCYEVKTKDPSGPPLDANIFQGKLYPEAKDHPEM | 101 |
| APOBEC-3G | KFLHWFRKWRQLHRDQEYEVTWYVSWSPCTRCANSVATFLAEDPKVTLTIFVARLYYFWKPDYQQALR | |
| | ILCQERGGPHATMKIMNYNEFQHCWNEFVDGQGKPFKPRKNLPKHYTLLHATLGELLRHVMDPGTFTS | |
| | NFNNKPWVSGQRETYLCYKVERSHNDTWVLLNQHRGFLRNQAPDRHGFPKGRHAELCFLDLIPFWKLD | |
| | DQQYRVTCFTSWSPCFSCAQKMAKFISNNKHVSLCIFAARIYDDQGRCQEGLRTLHRDGAKIAVMNYS | |
| | EFEYCWDTFVDRQGRPFQPWDGLDEHSQALSGRLRAI | |
| Human APOBEC- | MKPHFRNTVERMYRDTFSYNFYNRPILSRRNTVWLCYEVKTKGPSRPPLDAKIFRGQVYSELKYHPEM | 102 |
| 3G | RFFHWFSKWRKLHRDQEYEVTWYISWSPCTKCTRDMATFLAEDPKVTLTIFVARLYYFWDPDYQEALR | |
| | SLCQKRDGPRATMKIMNYDEFQHCWSKFVYSQRELFEPWNNLPKYYILLHIMLGEILRHSMDPPTFTF | |
| | NFNNEPWVRGRHETYLCYEVERMHNDTWVLLNQRRGFLCNQAPHKHGFLEGRHAELCFLDVIPFWKLD | |
| | LDQDYRVTCFTSWSPCFSCAQEMAKFISKNKHVSLCIFTARIYDDQGRCQEGLRTLAEAGAKISIMTY | |
| | SEFKHCWDTFVDHQGCPFQPWDGLDEHSQDLSGRLRAILQNQEN | |
| Human APOBEC- | MKPHFRNTVERMYRDTFSYNFYNRPILSRRNTVWLCYEVKTKGPSRPRLDAKIFRGQVYSQPEHHAEM | 103 |
| 3F | CFLSWFCGNQLPAYKCFQITWFVSWTPCPDCVAKLAEFLAEHPNVTLTISAARLYYYWERDYRRALCR | |
| | LSQAGARVKIMDDEEFAYCWENFVYSEGQPFMPWYKFDDNYAFLHRTLKEILRNPMEAMYPHIFYFHF | |
| | KNLRKAYGRNESWLCFTMEVVKHHSPVSWKRGVFRNQVDPETHCHAERCFLSWFCDDILSPNTNYEVT | |
| | WYTSWSPCPECAGEVAEFLARHSNVNLTIFTARLYYFWDTDYQEGLRSLSQEGASVEIMGYKDFKYCW | |
| | ENFVYNDDEPFKPWKGLKYNFLFLDSKLQEILE | |
| Human APOBEC- | MNPQIRNPMERMYRDTFYDNFENEPILYGRSYTWLCYEVKIKRGRSNLLWDTGVFRGQVYFKPQYHAE | 104 |
| 3B | MCFLSWFCGNQLPAYKCFQITWFVSWTPCPDCVAKLAEFLSEHPNVTLTISAARLYYYWERDYRRALC | |
| | RLSQAGARVTIMDYEEFAYCWENFVYNEGQQFMPWYKFDENYAFLHRTLKEILRYLMDPDTFTFNFNN | |
| | DPLVLRRRQTYLCYEVERLDNGTWVLMDQHMGFLCNEAKNLLCGFYGRHAELRFLDLVPSLQLDPAQI | |
| | YRVTWFISWSPCFSWGCAGEVRAFLQENTHVRLRIFAARIYDYDPLYKEALQMLRDAGAQVSIMTYDE | |
| | FEYCWDTFVYRQGCPFQPWDGLEEHSQALSGRLRAILQNQGN | |
| Rat APOBEC-3B | MQPQGLGPNAGMGPVCLGCSHRRPYSPIRNPLKKLYQQTFYFHFKNVRYAWGRKNNFLCYEVNGMDCA | 105 |
| | LPVPLRQGVFRKQGHIHAELCFIYWFHDKVLRVLSPMEEFKVTWYMSWSPCSKCAEQVARFLAAHRNL | |
| | SLAIFSSRLYYYLRNPNYQQKLCRLIQEGVHVAAMDLPEFKKCWNKFVDNDGQPFRPWMRLRINFSFY | |
| | DCKLQEIFSRMNLLREDVFYLQFNNSHRVKPVQNRYYRRKSYLCYQLERANGQEPLKGYLLYKKGEQH | |
| | VEILFLEKMRSMELSQVRITCYLTWSPCPNCARQLAAFKKDHPDLILRIYTSRLYFYWRKKFQKGLCT | |
| | LWRSGIHVDVMDLPQFADCWTNFVNPQRPFRPWNELEKNSWRIQRRLRRIKESWGL | |
| Bovine APOBEC- | DGWEVAFRSGTVLKAGVLGVSMTEGWAGSGHPGQGACVWTPGTRNTMNLLREVLFKQQFGNQPRVPAP | 106 |
| 3B | YYRRKTYLCYQLKQRNDLTLDRGCFRNKKQRHAEIRFIDKINSLDLNPSQSYKIICYITWSPCPNCAN | |
| | ELVNFITRNNHLKLEIFASRLYFHWIKSFKMGLQDLQNAGISVAVMTHTEFEDCWEQFVDNQSRPFQP | |
| | WDKLEQYSASIRRRLQRILTAPI | |
| Chimpanzee | MNPQIRNPMEWMYQRTFYYNFENEPILYGRSYTWLCYEVKIRRGHSNLLWDTGVFRGQMYSQPEHHAE | 107 |
| APOBEC-3B | MCFLSWFCGNQLSAYKCFQITWFVSWTPCPDCVAKLAKFLAEHPNVTLTISAARLYYYWERDYRRALC | |
| | RLSQAGARVKIMDDEEFAYCWENFVYNEGQPFMPWYKFDDNYAFLHRTLKEIIRHLMDPDTFTFNFNN | |
| | DPLVLRRHQTYLCYEVERLDNGTWVLMDQHMGFLCNEAKNLLCGFYGRHAELRFLDLVPSLQLDPAQI | |
| | YRVTWFISWSPCFSWGCAGQVRAFLQENTHVRLRIFAARIYDYDPLYKEALQMLRDAGAQVSIMTYDE | |
| | FEYCWDTFVYRQGCPFQPWDGLEEHSQALSGRLRAILQVRASSLCMVPHRPPPPPQSPGPCLPLCSEP | |
| | PLGSLLPTGRPAPSLPFLLTASFSFPPPASLPPLPSLSLSPGHLPVPSFHSLTSCSIQPPCSSRIRET | |
| | EGWASVSKEGRDLG | |
| Human APOBEC- | MNPQIRNPMKAMYPGTFYFQFKNLWEANDRNETWLCFTVEGIKRRSVVSWKTGVFRNQVDSETHCHAE | 108 |
| 3C | RCFLSWFCDDILSPNTKYQVTWYTSWSPCPDCAGEVAEFLARHSNVNLTIFTARLYYFQYPCYQEGLR | |
| | SLSQEGVAVEIMDYEDFKYCWENFVYNDNEPFKPWKGLKTNFRLLKRRLRESLQ | |
| Gorilla | MNPQIRNPMKAMYPGTFYFQFKNLWEANDRNETWLCFTVEGIKRRSVVSWKTGVFRNQVDSETHCHAE | 109 |
| APOBEC3C | RCFLSWFCDDILSPNTNYQVTWYTSWSPCPECAGEVAEFLARHSNVNLTIFTARLYYFQDTDYQEGLR | |
| | SLSQEGVAVKIMDYKDFKYCWENFVYNDDEPFKPWKGLKYNFRFLKRRLQEILE | |
| Human APOBEC- | MEASPASGPRHLMDPHIFTSNFNNGIGRHKTYLCYEVERLDNGTSVKMDQHRGFLHNQAKNLLCGFYG | 110 |
| 3A | RHAELRFLDLVPSLQLDPAQIYRVTWFISWSPCFSWGCAGEVRAFLQENTHVRLRIFAARIYDYDPLY | |
| | KEALQMLRDAGAQVSIMTYDEFKHCWDTFVDHQGCPFQPWDGLDEHSQALSGRLRAILQNQGN | |
| Rhesus macaque | MDGSPASRPRHLMDPNTFTFNFNNDLSVRGRHQTYLCYEVERLDNGTWVPMDERRGFLCNKAKNVPCG | 111 |
| APOBEC-3A | DYGCHVELRFLCEVPSWQLDPAQTYRVTWFISWSPCFRRGCAGQVRVFLQENKHVRLRIFAARIYDYD | |
| | PLYQEALRTLRDAGAQVSIMTYEEFKHCWDTFVDRQGRPFQPWDGLDEHSQALSGRLRAILQNQGN | |
| Bovine APOBEC- | MDEYTFTENFNNQGWPSKTYLCYEMERLDGDATIPLDEYKGFVRNKGLDQPEKPCHAELYFLGKIHSW | 112 |
| 3A | NLDRNQHYRLTCFISWSPCYDCAQKLTTFLKENHHISLHILASRIYTHNRFGCHQSGLCELQAAGARI | |
| | TIMTFEDFKHCWETFVDHKGKPFQPWEGLNVKSQALCTELQAILKTQQN | |
| Human APOBEC- | MALLTAETFRLQFNNKRRLRRPYYPRKALLCYQLTPQNGSTPTRGYFENKKKCHAEICFINEIKSMGL | 113 |
| 3H | DETQCYQVTCYLTWSPCSSCAWELVDFIKAHDHLNLGIFASRLYYHWCKPQQKGLRLLCGSQVPVEVM | |
| | GFPKFADCWENFVDHEKPLSFNPYKMLEELDKNSRAIKRRLERIKIPGVRAQGRYMDILCDAEV | |
| Rhesus macaque | MALLTAKTFSLQFNNKRRVNKPYYPRKALLCYQLTPQNGSTPTRGHLKNKKKDHAEIRFINKIKSMGL | 114 |
| APOBEC-3H | DETQCYQVTCYLTWSPCPSCAGELVDFIKAHRHLNLRIFASRLYYHWRPNYQEGLLLLCGSQVPVEVM | |
| | GLPEFTDCWENFVDHKEPPSFNPSEKLEELDKNSQAIKRRLERIKSRSVDVLENGLRSLQLGPVTPSS | |
| | SIRNSR | |
| Human APOBEC- | MNPQIRNPMERMYRDTFYDNFENEPILYGRSYTWLCYEVKIKRGRSNLLWDTGVFRGPVLPKRQSNHR | 115 |
| 3D | QEVYFRFENHAEMCFLSWFCGNRLPANRRFQITWFVSWNPCLPCVVKVTKFLAEHPNVTLTISAARLY | |
| | YYRDRDWRWVLLRLHKAGARVKIMDYEDFAYCWENFVCNEGQPFMPWYKFDDNYASLHRTLKEILRNP | |
| | MEAMYPHIFYFHFKNLLKACGRNESWLCFTMEVTKHHSAVFRKRGVFRNQVDPETHCHAERCFLSWFC | |
| | DDILSPNTNYEVTWYTSWSPCPECAGEVAEFLARHSNVNLTIFTARLCYFWDTDYQEGLCSLSQEGAS | |
| | VKIMGYKDFVSCWKNFVYSDDEPFKPWKGLQTNFRLLKRRLREILQ | |
| Human APOBEC-1 | MTSEKGPSTGDPTLRRRIEPWEFDVFYDPRELRKEACLLYEIKWGMSRKIWRSSGKNTTNHVEVNFIK | 116 |
| | KFTSERDFHPSMSCSITWFLSWSPCWECSQAIREFLSRHPGVTLVIYVARLFWHMDQQNRQGLRDLVN | |
| | SGVTIQIMRASEYYHCWRNFVNYPPGDEAHWPQYPPLWMMLYALELHCIILSLPPCLKISRRWQNHLT | |
| | FFRLHLQNCHYQTIPPHILLATGLIHPSVAWR | |
| Mouse APOBEC-1 | MSSETGPVAVDPTLRRRIEPHEFEVFFDPRELRKETCLLYEINWGGRHSVWRHTSQNTSNHVEVNFLE | 117 |
| | KFTTERYFRPNTRCSITWFLSWSPCGECSRAITEFLSRHPYVTLFIYIARLYHHTDQRNRQGLRDLIS | |
| | SGVTIQIMTEQEYCYCWRNFVNYPPSNEAYWPRYPHLWVKLYVLELYCIILGLPPCLKILRRKQPQLT | |
| | FFTITLQTCHYQRIPPHLLWATGLK | |
| Rat APOBEC-1 | MSSETGPVAVDPTLRRRIEPHEFEVFFDPRELRKETCLLYEINWGGRHSIWRHTSQNTNKHVEVNFIE | 118 |
| | KFTTERYFCPNTRCSITWFLSWSPCGECSRAITEFLSRYPHVTLFIYIARLYHHADPRNRQGLRDLIS | |
| | SGVTIQIMTEQESGYCWRNFVNYSPSNEAHWPRYPHLWVRLYVLELYCIILGLPPCLNILRRKQPQLT | |
| | FFTIALQSCHYQRLPPHILWATGLK | |
| Human APOBEC-2 | MAQKEEAAVATEAASQNGEDLENLDDPEKLKELIELPPFEIVTGERLPANFFKFQFRNVEYSSGRNKT | 119 |
| | FLCYVVEAQGKGGQVQASRGYLEDEHAAAHAEEAFFNTILPAFDPALRYNVTWYVSSSPCAACADRII | |
| | KTLSKTKNLRLLILVGRLFMWEEPEIQAALKKLKEAGCKLRIMKPQDFEYVWQNFVEQEEGESKAFQP | |
| | WEDIQENFLYYEEKLADILK | |
| Mouse APOBEC-2 | MAQKEEAAEAAAPASQNGDDLENLEDPEKLKELIDLPPFEIVTGVRLPVNFFKFQFRNVEYSSGRNKT | 120 |
| | FLCYVVEVQSKGGQAQATQGYLEDEHAGAHAEEAFFNTILPAFDPALKYNVTWYVSSSPCAACADRIL | |
| | KTLSKTKNLRLLILVSRLFMWEEPEVQAALKKLKEAGCKLRIMKPQDFEYIWQNFVEQEEGESKAFEP | |
| | WEDIQENFLYYEEKLADILK | |
| Rat APOBEC-2 | MAQKEEAAEAAAPASQNGDDLENLEDPEKLKELIDLPPFEIVTGVRLPVNFFKFQFRNVEYSSGRNKT | 121 |
| | FLCYVVEAQSKGGQVQATQGYLEDEHAGAHAEEAFFNTILPAFDPALKYNVTWYVSSSPCAACADRIL | |
| | KTLSKTKNLRLLILVSRLFMWEEPEVQAALKKLKEAGCKLRIMKPQDFEYLWQNFVEQEEGESKAFEP | |
| | WEDIQENFLYYEEKLADILK | |
| Bovine APOBEC- | MAQKEEAAAAAEPASQNGEEVENLEDPEKLKELIELPPFEIVTGERLPAHYFKFQFRNVEYSSGRNKT | 122 |
| 2 | FLCYVVEAQSKGGQVQASRGYLEDEHATNHAEEAFFNSIMPTFDPALRYMVTWYVSSSPCAACADRIV | |
| | KTLNKTKNLRLLILVGRLFMWEEPEIQAALRKLKEAGCRLRIMKPQDFEYIWQNFVEQEEGESKAFEP | |
| | WEDIQENFLYYEEKLADILK | |
| Petromyzon | MTDAEYVRIHEKLDIYTFKKQFFNNKKSVSHRCYVLFELKRRGERRACFWGYAVNKPQSGTERGIHAE | 123 |
| marinus CDA1 | IFSIRKVEEYLRDNPGQFTINWYSSWSPCADCAEKILEWYNQELRGNGHTLKIWACKLYYEKNARNQI | |
| (pmCDA1) | GLWNLRDNGVGLNVMVSEHYQCCRKIFIQSSHNQLNENRWLEKTLKRAEKRRSELSIMIQVKILHTTK | |
| | SPAV | |
| Human APOBEC3G | MKPHFRNTVERMYRDTFSYNFYNRPILSRRNTVWLCYEVKTKGPSRPPLDAKIFRGQVYSELKYHPEM | 124 |
| D316R_D317R | RFFHWFSKWRKLHRDQEYEVTWYISWSPCTKCTRDMATFLAEDPKVTLTIFVARLYYFWDPDYQEALR | |
| | SLCQKRDGPRATMKIMNYDEFQHCWSKFVYSQRELFEPWNNLPKYYILLHIMLGEILRHSMDPPTFTF | |
| | NFNNEPWVRGRHETYLCYEVERMHNDTWVLLNQRRGFLCNQAPHKHGFLEGRHAELCFLDVIPFWKLD | |
| | LDQDYRVTCFTSWSPCFSCAQEMAKFISKNKHVSLCIFTARIYRRQGRCQEGLRTLAEAGAKISIMTY | |
| | SEFKHCWDTFVDHQGCPFQPWDGLDEHSQDLSGRLRAILQNQEN | |
| Human APOBEC3G | MDPPTFTFNFNNEPWVRGRHETYLCYEVERMHNDTWVLLNQRRGFLCNQAPHKHGFLEGRHAELCFLD | 125 |
| chain A | VIPFWKLDLDQDYRVTCFTSWSPCFSCAQEMAKFISKNKHVSLCIFTARIYDDQGRCQEGLRTLAEAG | |
| | AKISIMTYSEFKHCWDTFVDHQGCPFQPWDGLDEHSQDLSGRLRAILQ | |
| Human APOBEC3G | MDPPTFTFNFNNEPWVRGRHETYLCYEVERMHNDTWVLLNQRRGFLCNQAPHKHGFLEGRHAELCFLD | 126 |
| chain A | VIPFWKLDLDQDYRVTCFTSWSPCFSCAQEMAKFISKNKHVSLCIFTARIYRRQGRCQEGLRTLAEAG | |
| D120R D121R | AKISIMTYSEFKHCWDTFVDHQGCPFQPWDGLDEHSQDLSGRLRAILQ | |
| FERNY | MFERNYDPRELRKETYLLYEIKWGKSGKLWRHWCQNNRTQHAEVYFLENIFNARRFNPSTHCSITWYL | 127 |
| | SWSPCAECSQKIVDFLKEHPNVNLEIYVARLYYHEDERNRQGLRDLVNSGVTIRIMDLPDYNYCWKTF | |
| | VSDQGGDEDYWPGHFAPWIKQYSLKL | |
| evoFERNY | MFERNYDPRELRKETYLLYEIKWGKSGKLWRHWCQNNRTQHAEVYFLENIFNARRFNPSTHCSITWYL | 128 |
| | SWSPCAECSQKIVDFLKEHPNVNLEIYVARLYYPENERNRQGLRDLVNSGVTIRIMDLPDYNYCWKTF | |
| | VSDQGGDEDYWPGHFAPWIKQYSLKL | |
| Rat APOBEC-1 | MSSETGPVAVDPTLRRRIEPHEFEVFFDPRELRKETCLLYEINWGGRHSIWRHTSQNTNKHVEVNFIE | 129 |
| | KFTTERYFCPNTRCSITWFLSWSPCGECSRAITEFLSRYPHVTLFIYIARLYHHADPRNRQGLRDLIS | |
| | SGVTIQIMTEQESGYCWRNFVNYSPSNEAHWPRYPHLWVRLYVLELYCIILGLPPCLNILRRKQPQLT | |
| | FFTIALQSCHYQRLPPHILWATGLK | |
| evoAPOBEC | MSSKTGPVAVDPTLRRRIEPHEFEVFFDPRELRKETCLLYEINWGGRHSIWRHTSQNTNKHVEVNFIE | 130 |
| | KFTTERYFCPNTRCSITWFLSWSPCGECSRAITEFLSRYPNVTLFIYIARLYHLANPRNRQGLRDLIS | |
| | SGVTIQIMTEQESGYCWHNFVNYSPSNESHWPRYPHLWVRLYVLELYCIILGLPPCLNILRRKQSQLT | |
| | SFTIALQSCHYQRLPPHILWATGLK | |
| Petromyzon | MTDAEYVRIHEKLDIYTFKKQFFNNKKSVSHRCYVLFELKRRGERRACFWGYAVNKPQSGTERGIHAE | 131 |
| marinus CDA1 | IFSIRKVEEYLRDNPGQFTINWYSSWSPCADCAEKILEWYNQELRGNGHTLKIWACKLYYEKNARNQI | |
| | GLWNLRDNGVGLNVMVSEHYQCCRKIFIQSSHNQLNENRWLEKTLKRAEKRRSELSIMIQVKILHTTK | |
| | SPAV | |
| evoCDA | MTDAEYVRIHEKLDIYTFKKQFSNNKKSVSHRCYVLFELKRRGERRACFWGYAVNKPQSGTERGIHAE | 132 |
| | IFSIRKVEEYLRDNPGQFTINWYSSWSPCADCAEKILEWYNQELRGNGHTLKIWVCKLYYEKNARNQI | |
| | GLWNLRDNGVGLNVMVSEHYQCCRKIFIQSSHNQLNENRWLEKTLKRAEKRRSELSIMFQVKILHTTK | |
| | SPAV | |
| Anc689 APOBEC | MSSETGPVAVDPTLRRRIEPHEFEVFFDPRELRKETCLLYEIKWGTSHKIWRHSSKNTTKHVEVNFIE | 133 |
| | KFTSERHFCPSTSCSITWFLSWSPCGECSKAITEFLSQHPNVTLVIYVARLYHHMDQQNRQGLRDLVN | |
| | SGVTIQIMTAPEYDYCWRNFVNYPPGKEAHWPRYPPLWMKLYALELHAGILGLPPCLNILRRKQPQLT | |
| | FFTIALQSCHYQRLPPHILWATGLK | |
| evoAnc68 9 | MSSKTGPVAVDPTLRRRIEPHEFEVFFDPRELRKETCLLYEIKWGTSHKIWRHSSKNTTKHVEVNFIE | 134 |
| APOBEC | KFTSERHFCPSTSCSITWFLSWSPCGECSKAITEFLSQHPNVTLVIYVARLYHLMNQQNRQGLRDLVN | |
| | SGVTIQIMTAPEYDYCWHNFVNYPPGKESHWPRYPPLWMKLYALELHAGILGLPPCLNILRRKQSQLT | |
| | SFTIALQSCHYQRLPPHILWATGLK | |

Linkers

| | | SEQ ID |
|---|---|---|
| DESCRIPTION | SEQUENCE | NO: |
| linker | (G)n | 135 |
| linker | (XP)n | 136 |
| linker | (GGS)n | 137 |
| linker | (SGGS) | 138 |
| linker | (SGGS)n | 139 |
| linker | (GGGS)n | 140 |
| linker | (GGGGS)n | 141 |
| linker | (EAAAK)n | 142 |
| XTEN linker | (SGSETPGTSESATPES) | 143 |
| (SGGS)2-XTEN- | (SGGS)2-SGSETPGTSESATPES-(SGGS)2 | 144 |
| (SGGS)2 linker | | |
| linker | (SGGS)nSGSETPGTSESATPES(SGGS)n | 145 |
| linker | (SGGSSGGSSGSETPGTSESATPES) | 146 |
| linker | (SGGSSGGSSGSETPGTSESATPESSGGSSGGS) | 147 |
| linker | (SGGSSGGSSGSETPGTSESATPESSGGSSGGSSGGSSGGS) | 148 |
| linker | (SGGSSGGSSGSETPGTSESATPESSGGSSGGSSGGSSGGSSGSETPGTSESATPESSGGSSGGS) | 149 |
| linker | (PGSPAGSPTSTEEGTSESATPESGPGTSTEPSEGSAPGSPAGSPTSTEEGTSTEPSEGSAPGTSTEP | 150 |
| | SEGSAPGTSESATPESGPGSEPATS) | |
| linker | (GGSGGSPGSPAGSPTSTEEGTSESATPESGPGTSTEPSEGSAPGSPAGSPTSTEEGTSTEPSEGSAP | 151 |
| | GTSTEPSEGSAPGTSESATPESGPGSEPATSGGSGGS) | |

NLS

| | | SEQ ID |
|---|---|---|
| DESCRIPTION | SEQUENCE | NO: |
| NLS of SV40 | PKKKRKV | 152 |
| large T-Ag | | |
| NLS of polyoma | VSRKRPRP | 153 |
| large T-Ag | | |
| NLS of TUS- | KLKIKRPVK | 155 |
| protein | | |
| NLS of c-MYC | PAAKRVKLD | 154 |
| NLS of | EGAPPAKRAR | 156 |
| Hepatitis D | | |
| virus antigen | | |
| NLS of murine | PPQPKKKPLDGE | 157 |
| p53 | | |
| NLS | MKRTADGSEFESPKKKRKV | 158 |
| NLS of | AVKRPAATKKAGQAKKKKLD | 159 |
| nucleoplasmin | | |
| NLS | SGGSKRTADGSEFEPKKKRKV | 160 |
| NLS of EGL-13 | MSRRRKANPTKLSENAKKLAKEVEN | 161 |
| NLS | MDSLLMNRRKFLYQFKNVRWAKGRRETYLC | 162 |

UGI

| | | SEQ ID |
|---|---|---|
| DESCRIPTION | SEQUENCE | NO: |
| UGI- | MTNLSDIIEKETGKQLVIQESILMLPEEVEEVIGNKPESDILVHTAYDESTD | 163 |
| sp\|P14739\|UNGI | ENVMLLTSDAPEYKPWALVIQDSNGENKIKML | |
| BPPB2 | | |

Intein

| | | SEQ ID |
|---|---|---|
| DESCRIPTION | SEQUENCE | NO: |
| 2-4 intein | CLAEGTRIFDPVTGTTHRIEDVVDGRKPIHVVAAAKDGTLLARPVVSWFDQGTRDVIGLRIAGGAIVW | 164 |
| | ATPDHKVLTEYGWRAAGELRKGDRVAGPGGSGNSLALSLTADQMVSALLDAEPP1LYSEYDPTSPFSE | |
| | ASMMGLLTNLADRELVHMINWAKRVPGFVDLTLHDQAHLLECAWLEILMIGLVWRSMEHPGKLLFAPN | |
| | LLLDRNQGKCVEGMVEIFDMLLATSSRFRMMNLQGEEFVCLKSIILLNSGVYTFLSSTLKSLEEKDHI | |
| | HRALDKITDTLIHLMAKAGLTLQQQHQRLAQLLLILSHIRHMSNKGMEHLYSMKYKNVVPLYDLLLEM | |
| | LDAHRLHAGGSGASRVQAFADALDDKFLHDMLAEELRYSVIREVLPTRRARTFDLEVEELHTLVAEGV | |
| | WHNC | |
| 3-2 intein | CLAEGTRIFDPVTGTTHRIEDVVDGRKPIHVVAVAKDGTLLARPVVSWFDQGTRDVIGLRIAGGAIVW | 165 |
| | ATPDHKVLTEYGWRAAGELRKGDRVAGPGGSGNSLALSLTADQMVSALLDAEPPILYSEYDPTSPFSE | |
| | ASMMGLLTNLADRELVHMINWAKRVPGFVDLTLHDQAHLLECAWLEILMIGLVWRSMEHPGKLLFAPN | |
| | LLLDRNQGKCVEGMVEIFDMLLATSSRFRMMNLQGEEFVCLKSIILLNSGVYTFLSSTLKSLEEKDHI | |
| | HRALDKITDTLIHLMAKAGLTLQQQHQRLAQLLLILSHIRHMSNKGMEHLYSMKYTNVVPLYDLLLEM | |
| | LDAHRLHAGGSGASRVQAFADALDDKFLHDMLAEELRYSVIREVLPTRRARTFDLEVEELHTLVAEGV | |
| | WHNC | |
| 30R3-1 intein | CLAEGTRIFDPVTGTTHRIEDVVDGRKPIHVVAAAKDGTLLARPVVSWFDQGTRDVIGLRIAGGATVW | 166 |
| | ATPDHKVLTEYGWRAAGELRKGDRVAGPGGSGNSLALSLTADQMVSALLDAEPPIPYSEYDPTSPFSE | |
| | ASMMGLLTNLADRELVHMINWAKRVPGFVDLTLHDQAHLLECAWLEILMIGLVWRSMEHPGKLLFAPN | |
| | LLLDRNQGKCVEGMVEIFDMLLATSSRFRMMNLQGEEFVCLKSIILLNSGVYTFLSSTLKSLEEKDHI | |
| | HRALDKITDTLIHLMAKAGLTLQQQHQRLAQLLLILSHIRHMSNKGMEHLYSMKYKNVVPLYDLLLEM | |
| | LDAHRLHAGGSGASRVQAFADALDDKFLHDMLAEGLRYSVIREVLPTRRARTFDLEVEELHTLVAEGV | |
| | WHNC | |
| 30R3-2 intein | CLAEGTRIFDPVTGTTHRIEDVVDGRKPIHVVAAAKDGTLLARPVVSWFDQGTRDVIGLRIAGGATVW | 167 |
| | ATPDHKVLTEYGWRAAGELRKGDRVAGPGGSGNSLALSLTADQMVSALLDAEPPILYSEYDPTSPFSE | |
| | ASMMGLLTNLADRELVHMINWAKRVPGFVDLTLHDQAHLLECAWLEILMIGLVWRSMEHPGKLLFAPN | |
| | LLLDRNQGKCVEGMVEIFDMLLATSSRFRMMNLQGEEFVCLKSIILLNSGVYTFLSSTLKSLEEKDHI | |
| | HRALDKITDTLIHLMAKAGLTLQQQHQRLAQLLLILSHIRHMSNKGMEHLYSMKYKNVVPLYDLLLEM | |
| | LDAHRLHAGGSGASRVQAFADALDDKFLHDMLAEELRYSVIREVLPTRRARTFDLEVEELHTLVAEGV | |
| | WHNC | |
| 30R3-3 intein | CLAEGTRIFDPVTGTTHRIEDVVDGRKPIHVVAAAKDGTLLARPVVSWFDQGTRDVIGLRIAGGATVW | 168 |
| | ATPDHKVLTEYGWRAAGELRKGDRVAGPGGSGNSLALSLTADQMVSALLDAEPPIPYSEYDPTSPFSE | |
| | ASMMGLLTNLADRELVHMINWAKRVPGFVDLTLHDQAHLLECAWLEILMIGLVWRSMEHPGKLLFAPN | |
| | LLLDRNQGKCVEGMVEIFDMLLATSSRFRMMNLQGEEFVCLKSIILLNSGVYTFLSSTLKSLEEKDHI | |
| | HRALDKITDTLIHLMAKAGLTLQQQHQRLAQLLLILSHIRHMSNKGMEHLYSMKYKNVVPLYDLLLEM | |
| | LDAHRLHAGGSGASRVQAFADALDDKFLHDMLAEELRYSVIREVLPTRRARTFDLEVEELHTLVAEGV | |
| | WHNC | |
| 37R3-1 intein | CLAEGTRIFDPVTGTTHRIEDVVDGRKPIHVVAAAKDGTLLARPVVSWFDQGTRDVIGLRIAGGATVW | 169 |
| | ATPDHKVLTEYGWRAAGELRKGDRVAGPGGSGNSLALSLTADQMVSALLDAEPPILYSEYNPTSPFSE | |
| | ASMMGLLTNLADRELVHMINWAKRVPGFVDLTLHDQAHLLERAWLEILMIGLVWRSMEHPGKLLFAPN | |
| | LLLDRNQGKCVEGMVEIFDMLLATSSRFRMMNLQGEEFVCLKSIILLNSGVYTFLSSTLKSLEEKDHI | |
| | HRALDKITDTLIHLMAKAGLTLQQQHQRLAQLLLILSHIRHMSNKGMEHLYSMKYKNVVPLYDLLLEM | |
| | LDAHRLHAGGSGASRVQAFADALDDKFLHDMLAEGLRYSVIREVLPTRRARTFDLEVEELHTLVAEGV | |
| | WHNC | |
| 37R3-2 intein | CLAEGTRIFDPVTGTTHRIEDVVDGRKPIHVVAAAKDGTLLARPVVSWFDQGTRDVIGLRIAGGAIVW | 170 |
| | ATPDHKVLTEYGWRAAGELRKGDRVAGPGGSGNSLALSLTADQMVSALLDAEPPILYSEYDPTSPFSE | |
| | ASMMGLLTNLADRELVHMINWAKRVPGFVDLTLHDQAHLLERAWLEILMIGLVWRSMEHPGKLLFAPN | |
| | LLLDRNQGKCVEGMVEIFDMLLATSSRFRMMNLQGEEFVCLKSIILLNSGVYTFLSSTLKSLEEKDHI | |
| | HRALDKITDTLIHLMAKAGLTLQQQHQRLAQLLLILSHIRHMSNKGMEHLYSMKYKNVVPLYDLLLEM | |
| | LDAHRLHAGGSGASRVQAFADALDDKFLHDMLAEGLRYSVIREVLPTRRARTFDLEVEELHTLVAEGV | |
| | WHNC | |
| 37R3-3 intein | CLAEGTRIFDPVTGTTHRIEDVVDGRKPIHVVAVAKDGTLLARPVVSWFDQGTRDVIGLRIAGGATVW | 171 |
| | ATPDHKVLTEYGWRAAGELRKGDRVAGPGGSGNSLALSLTADQMVSALLDAEPPILYSEYDPTSPFSE | |
| | ASMMGLLTNLADRELVHMINWAKRVPGFVDLTLHDQAHLLERAWLEILMIGLVWRSMEHPGKLLFAPN | |
| | LLLDRNQGKCVEGMVEIFDMLLATSSRFRMMNLQGEEFVCLKSIILLNSGVYTFLSSTLKSLEEKDHI | |
| | HRALDKITDTLIHLMAKAGLTLQQQHQRLAQLLLILSHIRHMSNKGMEHLYSMKYKNVVPLYDLLLEM | |
| | LDAHRLHAGGSGASRVQAFADALDDKFLHDMLAEELRYSVIREVLPTRRARTFDLEVEELHTLVAEGV | |
| | WHNC | |

RNA-protein recruitment system

| | | SEQ ID |
|---|---|---|
| DESCRIPTION | SEQUENCE | NO: |
| MS2 hairpin or | GCCAACATGAGGATCACCCATGTCTGCAGGGCC | 172 |
| aptamer | | |
| MCP or MS2cp | GSASNFTQFVLVDNGGTGDVTVAPSNFANGVAEWISSNSRSQAYKVTCSVRQSSAQNRKYTIKVEVPK | 173 |
| | VATQTVGGEELPVAGWRSYLNMELTIPIFATNSDCELIVKAMQGLLKDGNPIPSAIAANSGIY | |

ABEs (adenosine base editors)

| | | SEQ ID |
|---|---|---|
| DESCRIPTION | SEQUENCE | NO: |
| ecTadA(wt)- | MSEVEFSHEYWMRHALTLAKRAWDEREVPVGAVLVHNNRVIGEGWNRPIGRHDPTAHAEIMALRQGGL | 174 |
| XTEN-nCas9-NLS | VMQNYRLIDATLYVTLEPCVMCAGAMIHSRIGRVVFGARDAKTGAAGSLMDVLHHPGMNHRVEITEGI | |
| | LADECAALLSDFFRMRRQEIKAQKKAQSSTDSGSETPGTSESATPESDKKYSIGLAIGTNSVGWAVIT | |
| | DEYKVPSKKFKVLGNTDRHSIKKNLIGALLFDSGETAEATRLKRTARRRYTRRKNRICYLQEIFSNEM | |
| | AKVDDSFFHRLEESFLVEEDKKHERHPIFGNIVDEVAYHEKYPTIYHLRKKLVDSTDKADLRLIYLAL | |
| | AHMIKFRGHFLIEGDLNPDNSDVDKLFIQLVQTYNQLFEENPINASGVDAKAILSARLSKSRRLENLI | |
| | AQLPGEKKNGLFGNLIALSLGLTPNFKSNFDLAEDAKLQLSKDTYDDDLDNLLAQIGDQYADLFLAAK | |
| | NLSDAILLSDILRVNTEITKAPLSASMIKRYDEHHQDLTLLKALVRQQLPEKYKEIFFDQSKNGYAGY | |
| | IDGGASQEEFYKFIKPILEKMDGTEELLVKLNREDLLRKQRTFDNGSIPHQIHLGELHAILRRQEDFY | |
| | PFLKDNREKIEKILTFRIPYYVGPLARGNSRFAWMTRKSEETITPWNFEEVVDKGASAQSFIERMTNF | |
| | DKNLPNEKVLPKHSLLYEYFTVYNELTKVKYVTEGMRKPAFLSGEQKKAIVDLLFKTNRKVTVKQLKE | |
| | DYFKKIECFDSVEISGVEDRFNASLGTYHDLLKIIKDKDFLDNEENEDILEDIVLTLTLFEDREMIEE | |
| | RLKTYAHLFDDKVMKQLKRRRYTGWGRLSRKLINGIRDKQSGKTILDFLKSDGFANRNFMQLIHDDSL | |
| | TFKEDIQKAQVSGQGDSLHEHIANLAGSPAIKKGILQTVKVVDELVKVMGRHKPENIVIEMARENQTT | |
| | QKGQKNSRERMKRIEEGIKELGSQILKEHPVENTQLQNEKLYLYYLQNGRDMYVDQELDINRLSDYDV | |
| | DHIVPQSFLKDDSIDNKVLTRSDKNRGKSDNVPSEEVVKKMKNYWRQLLNAKLITQRKFDNLTKAERG | |
| | GLSELDKAGFIKRQLVETRQITKHVAQILDSRMNTKYDENDKLIREVKVITLKSKLVSDFRKDFQFYK | |
| | VREINNYHHAHDAYLNAVVGTALIKKYPKLESEFVYGDYKVYDVRKMIAKSEQEIGKATAKYFFYSNI | |
| | MNFFKTEITLANGEIRKRPLIETNGETGEIVWDKGRDFATVRKVLSMPQVNIVKKTEVQTGGFSKESI | |
| | LPKRNSDKLIARKKDWDPKKYGGFDSPTVAYSVLVVAKVEKGKSKKLKSVKELLGITIMERSSFEKNP | |
| | IDFLEAKGYKEVKKDLIIKLPKYSLFELENGRKRMLASAGELQKGNELALPSKYVNFLYLASHYEKLK | |
| | GSPEDNEQKQLFVEQHKHYLDEIIEQISEFSKRVILADANLDKVLSAYNKHRDKPIREQAENIIHLFT | |
| | LTNLGAPAAFKYFDTTIDRKRYTSTKEVLDATLIHQSITGLYETRIDLSQLGGDSGGSPKKKRKV | |
| ecTadA(D108N)- | MSEVEFSHEYWMRHALTLAKRAWDEREVPVGAVLVHNNRVIGEGWNRPIGRHDPTAHAEIMALRQGGL | 175 |
| XTEN-nCas9-NLS | VMQNYRLIDATLYVTLEPCVMCAGAMIHSRIGRVVFGARNAKTGAAGSLMDVLHHPGMNHRVEITEGI | |
| | LADECAALLSDFFRMRRQEIKAQKKAQSSTDSGSETPGTSESATPESDKKYSIGLAIGTNSVGWAVIT | |
| | DEYKVPSKKFKVLGNTDRHSIKKNLIGALLFDSGETAEATRLKRTARRRYTRRKNRICYLQEIFSNEM | |
| | AKVDDSFFHRLEESFLVEEDKKHERHPIFGNIVDEVAYHEKYPTIYHLRKKLVDSTDKADLRLIYLAL | |
| | AHMIKFRGHFLIEGDLNPDNSDVDKLFIQLVQTYNQLFEENPINASGVDAKAILSARLSKSRRLENLI | |
| | AQLPGEKKNGLFGNLIALSLGLTPNFKSNFDLAEDAKLQLSKDTYDDDLDNLLAQIGDQYADLFLAAK | |
| | NLSDAILLSDILRVNTEITKAPLSASMIKRYDEHHQDLTLLKALVRQQLPEKYKEIFFDQSKNGYAGY | |
| | IDGGASQEEFYKFIKPILEKMDGTEELLVKLNREDLLRKQRTFDNGSIPHQIHLGELHAILRRQEDFY | |
| | PFLKDNREKIEKILTFRIPYYVGPLARGNSRFAWMTRKSEETITPWNFEEVVDKGASAQSFIERMTNF | |
| | DKNLPNEKVLPKHSLLYEYFTVYNELTKVKYVTEGMRKPAFLSGEQKKAIVDLLFKTNRKVTVKQLKE | |
| | DYFKKIECFDSVEISGVEDRFNASLGTYHDLLKIIKDKDFLDNEENEDILEDIVLTLTLFEDREMIEE | |
| | RLKTYAHLFDDKVMKQLKRRRYTGWGRLSRKLINGIRDKQSGKTILDFLKSDGFANRNFMQLIHDDSL | |
| | TFKEDIQKAQVSGQGDSLHEHIANLAGSPAIKKGILQTVKVVDELVKVMGRHKPENIVIEMARENQTT | |
| | QKGQKNSRERMKRIEEGIKELGSQILKEHPVENTQLQNEKLYLYYLQNGRDMYVDQELDINRLSDYDV | |
| | DHIVPQSFLKDDSIDNKVLTRSDKNRGKSDNVPSEEVVKKMKNYWRQLLNAKLITQRKFDNLTKAERG | |
| | GLSELDKAGFIKRQLVETRQITKHVAQILDSRMNTKYDENDKLIREVKVITLKSKLVSDFRKDFQFYK | |
| | VREINNYHHAHDAYLNAVVGTALIKKYPKLESEFVYGDYKVYDVRKMIAKSEQEIGKATAKYFFYSNI | |
| | MNFFKTEITLANGEIRKRPLIETNGETGEIVWDKGRDFATVRKVLSMPQVNIVKKTEVQTGGFSKESI | |
| | LPKRNSDKLIARKKDWDPKKYGGFDSPTVAYSVLVVAKVEKGKSKKLKSVKELLGITIMERSSFEKNP | |
| | IDFLEAKGYKEVKKDLIIKLPKYSLFELENGRKRMLASAGELQKGNELALPSKYVNFLYLASHYEKLK | |
| | GSPEDNEQKQLFVEQHKHYLDEIIEQISEFSKRVILADANLDKVLSAYNKHRDKPIREQAENIIHLFT | |
| | LTNLGAPAAFKYFDTTIDRKRYTSTKEVLDATLIHQSITGLYETRIDLSQLGGDSGGSPKKKRKV | |
| ecTadA(D108G)- | MSEVEFSHEYWMRHALTLAKRAWDEREVPVGAVLVHNNRVIGEGWNRPIGRHDPTAHAEIMALRQGGL | 176 |
| XTEN-nCas9-NLS | VMQNYRLIDATLYVTLEPCVMCAGAMIHSRIGRVVFGARGAKTGAAGSLMDVLHHPGMNHRVEITEGI | |
| | LADECAALLSDFFRMRRQEIKAQKKAQSSTDSGSETPGTSESATPESDKKYSIGLAIGTNSVGWAVIT | |
| | DEYKVPSKKFKVLGNTDRHSIKKNLIGALLFDSGETAEATRLKRTARRRYTRRKNRICYLQEIFSNEM | |
| | AKVDDSFFHRLEESFLVEEDKKHERHPIFGNIVDEVAYHEKYPTIYHLRKKLVDSTDKADLRLIYLAL | |
| | AHMIKFRGHFLIEGDLNPDNSDVDKLFIQLVQTYNQLFEENPINASGVDAKAILSARLSKSRRLENLI | |
| | AQLPGEKKNGLFGNLIALSLGLTPNFKSNFDLAEDAKLQLSKDTYDDDLDNLLAQIGDQYADLFLAAK | |
| | NLSDAILLSDILRVNTEITKAPLSASMIKRYDEHHQDLTLLKALVRQQLPEKYKEIFFDQSKNGYAGY | |
| | IDGGASQEEFYKFIKPILEKMDGTEELLVKLNREDLLRKQRTFDNGSIPHQIHLGELHAILRRQEDFY | |
| | PFLKDNREKIEKILTFRIPYYVGPLARGNSRFAWMTRKSEETITPWNFEEVVDKGASAQSFIERMTNF | |
| | DKNLPNEKVLPKHSLLYEYFTVYNELTKVKYVTEGMRKPAFLSGEQKKAIVDLLFKTNRKVTVKQLKE | |
| | DYFKKIECFDSVEISGVEDRFNASLGTYHDLLKIIKDKDFLDNEENEDILEDIVLTLTLFEDREMIEE | |
| | RLKTYAHLFDDKVMKQLKRRRYTGWGRLSRKLINGIRDKQSGKTILDFLKSDGFANRNFMQLIHDDSL | |
| | TFKEDIQKAQVSGQGDSLHEHIANLAGSPAIKKGILQTVKVVDELVKVMGRHKPENIVIEMARENQTT | |
| | QKGQKNSRERMKRIEEGIKELGSQILKEHPVENTQLQNEKLYLYYLQNGRDMYVDQELDINRLSDYDV | |
| | DHIVPQSFLKDDSIDNKVLTRSDKNRGKSDNVPSEEVVKKMKNYWRQLLNAKLITQRKFDNLTKAERG | |
| | GLSELDKAGFIKRQLVETRQITKHVAQILDSRMNTKYDENDKLIREVKVITLKSKLVSDFRKDFQFYK | |
| | VREINNYHHAHDAYLNAVVGTALIKKYPKLESEFVYGDYKVYDVRKMIAKSEQEIGKATAKYFFYSNI | |
| | MNFFKTEITLANGEIRKRPLIETNGETGEIVWDKGRDFATVRKVLSMPQVNIVKKTEVQTGGFSKESI | |
| | LPKRNSDKLIARKKDWDPKKYGGFDSPTVAYSVLVVAKVEKGKSKKLKSVKELLGITIMERSSFEKNP | |
| | IDFLEAKGYKEVKKDLIIKLPKYSLFELENGRKRMLASAGELQKGNELALPSKYVNFLYLASHYEKLK | |
| | GSPEDNEQKQLFVEQHKHYLDEIIEQISEFSKRVILADANLDKVLSAYNKHRDKPIREQAENIIHLFT | |
| | LTNLGAPAAFKYFDTTIDRKRYTSTKEVLDATLIHQSITGLYETRIDLSQLGGDSGGSPKKKRKV | |
| ecTadA(D108V)- | MSEVEFSHEYWMRHALTLAKRAWDEREVPVGAVLVHNNRVIGEGWNRPIGRHDPTAHAEIMALRQGGL | 177 |
| XTEN-nCas9-NLS | VMQNYRLIDATLYVTLEPCVMCAGAMIHSRIGRVVFGARVAKTGAAGSLMDVLHHPGMNHRVEITEGI | |
| | LADECAALLSDFFRMRRQEIKAQKKAQSSTDSGSETPGTSESATPESDKKYSIGLAIGTNSVGWAVIT | |
| | DEYKVPSKKFKVLGNTDRHSIKKNLIGALLFDSGETAEATRLKRTARRRYTRRKNRICYLQEIFSNEM | |
| | AKVDDSFFHRLEESFLVEEDKKHERHPIFGNIVDEVAYHEKYPTIYHLRKKLVDSTDKADLRLIYLAL | |
| | AHMIKFRGHFLIEGDLNPDNSDVDKLFIQLVQTYNQLFEENPINASGVDAKAILSARLSKSRRLENLI | |
| | AQLPGEKKNGLFGNLIALSLGLTPNFKSNFDLAEDAKLQLSKDTYDDDLDNLLAQIGDQYADLFLAAK | |
| | NLSDAILLSDILRVNTEITKAPLSASMIKRYDEHHQDLTLLKALVRQQLPEKYKEIFFDQSKNGYAGY | |
| | IDGGASQEEFYKFIKPILEKMDGTEELLVKLNREDLLRKQRTFDNGSIPHQIHLGELHAILRRQEDFY | |
| | PFLKDNREKIEKILTFRIPYYVGPLARGNSRFAWMTRKSEETITPWNFEEVVDKGASAQSFIERMTNF | |
| | DKNLPNEKVLPKHSLLYEYFTVYNELTKVKYVTEGMRKPAFLSGEQKKAIVDLLFKTNRKVTVKQLKE | |
| | DYFKKIECFDSVEISGVEDRFNASLGTYHDLLKIIKDKDFLDNEENEDILEDIVLTLTLFEDREMIEE | |
| | RLKTYAHLFDDKVMKQLKRRRYTGWGRLSRKLINGIRDKQSGKTILDFLKSDGFANRNFMQLIHDDSL | |
| | TFKEDIQKAQVSGQGDSLHEHIANLAGSPAIKKGILQTVKVVDELVKVMGRHKPENIVIEMARENQTT | |
| | QKGQKNSRERMKRIEEGIKELGSQILKEHPVENTQLQNEKLYLYYLQNGRDMYVDQELDINRLSDYDV | |
| | DHIVPQSFLKDDSIDNKVLTRSDKNRGKSDNVPSEEVVKKMKNYWRQLLNAKLITQRKFDNLTKAERG | |
| | GLSELDKAGFIKRQLVETRQITKHVAQILDSRMNTKYDENDKLIREVKVITLKSKLVSDFRKDFQFYK | |
| | VREINNYHHAHDAYLNAVVGTALIKKYPKLESEFVYGDYKVYDVRKMIAKSEQEIGKATAKYFFYSNI | |
| | MNFFKTEITLANGEIRKRPLIETNGETGEIVWDKGRDFATVRKVLSMPQVNIVKKTEVQTGGFSKESI | |
| | LPKRNSDKLIARKKDWDPKKYGGFDSPTVAYSVLVVAKVEKGKSKKLKSVKELLGITIMERSSFEKNP | |
| | IDFLEAKGYKEVKKDLIIKLPKYSLFELENGRKRMLASAGELQKGNELALPSKYVNFLYLASHYEKLK | |
| | GSPEDNEQKQLFVEQHKHYLDEIIEQISEFSKRVILADANLDKVLSAYNKHRDKPIREQAENIIHLFT | |
| | LTNLGAPAAFKYFDTTIDRKRYTSTKEVLDATLIHQSITGLYETRIDLSQLGGDSGGSPKKKRKV | |
| ecTadA(H8Y_ | MSEVEFSYEYWMRHALTLAKRAWDEREVPVGAVLVHNNRVIGEGWNRPIGRHDPTAHAEIMALRQGGL | 178 |
| D108N_N127S)- | VMQNYRLIDATLYVTLEPCVMCAGAMIHSRIGRVVFGARNAKTGAAGSLMDVLHHPGMSHRVEITEGI | |
| XTEN-dCas9 | LADECAALLSDFFRMRRQEIKAQKKAQSSTDSGSETPGTSESATPESDKKYSIGLAIGTNSVGWAVIT | |
| | DEYKVPSKKFKVLGNTDRHSIKKNLIGALLFDSGETAEATRLKRTARRRYTRRKNRICYLQEIFSNEM | |
| | AKVDDSFFHRLEESFLVEEDKKHERHPIFGNIVDEVAYHEKYPTIYHLRKKLVDSTDKADLRLIYLAL | |
| | AHMIKFRGHFLIEGDLNPDNSDVDKLFIQLVQTYNQLFEENPINASGVDAKAILSARLSKSRRLENLI | |
| | AQLPGEKKNGLFGNLIALSLGLTPNFKSNFDLAEDAKLQLSKDTYDDDLDNLLAQIGDQYADLFLAAK | |
| | NLSDAILLSDILRVNTEITKAPLSASMIKRYDEHHQDLTLLKALVRQQLPEKYKEIFFDQSKNGYAGY | |
| | IDGGASQEEFYKFIKPILEKMDGTEELLVKLNREDLLRKQRTFDNGSIPHQIHLGELHAILRRQEDFY | |
| | PFLKDNREKIEKILTFRIPYYVGPLARGNSRFAWMTRKSEETITPWNFEEVVDKGASAQSFIERMTNF | |
| | DKNLPNEKVLPKHSLLYEYFTVYNELTKVKYVTEGMRKPAFLSGEQKKAIVDLLFKTNRKVTVKQLKE | |
| | DYFKKIECFDSVEISGVEDRFNASLGTYHDLLKIIKDKDFLDNEENEDILEDIVLTLTLFEDREMIEE | |
| | RLKTYAHLFDDKVMKQLKRRRYTGWGRLSRKLINGIRDKQSGKTILDFLKSDGFANRNFMQLIHDDSL | |
| | TFKEDIQKAQVSGQGDSLHEHIANLAGSPAIKKGILQTVKVVDELVKVMGRHKPENIVIEMARENQTT | |
| | QKGQKNSRERMKRIEEGIKELGSQILKEHPVENTQLQNEKLYLYYLQNGRDMYVDQELDINRLSDYDV | |
| | DAIVPQSFLKDDSIDNKVLTRSDKNRGKSDNVPSEEVVKKMKNYWRQLLNAKLITQRKFDNLTKAERG | |
| | GLSELDKAGFIKRQLVETRQITKHVAQILDSRMNTKYDENDKLIREVKVITLKSKLVSDFRKDFQFYK | |
| | VREINNYHHAHDAYLNAVVGTALIKKYPKLESEFVYGDYKVYDVRKMIAKSEQEIGKATAKYFFYSNI | |
| | MNFFKTEITLANGEIRKRPLIETNGETGEIVWDKGRDFATVRKVLSMPQVNIVKKTEVQTGGFSKESI | |
| | LPKRNSDKLIARKKDWDPKKYGGFDSPTVAYSVLVVAKVEKGKSKKLKSVKELLGITIMERSSFEKNP | |
| | IDFLEAKGYKEVKKDLIIKLPKYSLFELENGRKRMLASAGELQKGNELALPSKYVNFLYLASHYEKLK | |
| | GSPEDNEQKQLFVEQHKHYLDEIIEQISEFSKRVILADANLDKVLSAYNKHRDKPIREQAENIIHLFT | |
| | LTNLGAPAAFKYFDTTIDRKRYTSTKEVLDATLIHQSITGLYETRIDLSQLGGD | |
| (H8Y_D108N_ | MSEVEFSYEYWMRHALTLAKRAWDEREVPVGAVLVHNNRVIGEGWNRPIGRHDPTAHAEIMALRQGGL | 179 |
| N127S_E155X)- | VMQNYRLIDATLYVTLEPCVMCAGAMIHSRIGRVVFGARNAKTGAAGSLMDVLHHPGMSHRVEITEGI | |
| XTEN-dCas9; | LADECAALLSDFFRMRRQXIKAQKKAQSSTDSGSETPGTSESATPESDKKYSIGLAIGTNSVGWAVIT | |
| X = D, G or V | DEYKVPSKKFKVLGNTDRHSIKKNLIGALLFDSGETAEATRLKRTARRRYTRRKNRICYLQEIFSNEM | |
| | AKVDDSFFHRLEESFLVEEDKKHERHPIFGNIVDEVAYHEKYPTIYHLRKKLVDSTDKADLRLIYLAL | |
| | AHMIKFRGHFLIEGDLNPDNSDVDKLFIQLVQTYNQLFEENPINASGVDAKAILSARLSKSRRLENLI | |
| | AQLPGEKKNGLFGNLIALSLGLTPNFKSNFDLAEDAKLQLSKDTYDDDLDNLLAQIGDQYADLFLAAK | |
| | NLSDAILLSDILRVNTEITKAPLSASMIKRYDEHHQDLTLLKALVRQQLPEKYKEIFFDQSKNGYAGY | |
| | IDGGASQEEFYKFIKPILEKMDGTEELLVKLNREDLLRKQRTFDNGSIPHQIHLGELHAILRRQEDFY | |
| | PFLKDNREKIEKILTFRIPYYVGPLARGNSRFAWMTRKSEETITPWNFEEVVDKGASAQSFIERMTNF | |
| | DKNLPNEKVLPKHSLLYEYFTVYNELTKVKYVTEGMRKPAFLSGEQKKAIVDLLFKTNRKVTVKQLKE | |
| | DYFKKIECFDSVEISGVEDRFNASLGTYHDLLKIIKDKDFLDNEENEDILEDIVLTLTLFEDREMIEE | |
| | RLKTYAHLFDDKVMKQLKRRRYTGWGRLSRKLINGIRDKQSGKTILDFLKSDGFANRNFMQLIHDDSL | |
| | TFKEDIQKAQVSGQGDSLHEHIANLAGSPAIKKGILQTVKVVDELVKVMGRHKPENIVIEMARENQTT | |
| | QKGQKNSRERMKRIEEGIKELGSQILKEHPVENTQLQNEKLYLYYLQNGRDMYVDQELDINRLSDYDV | |
| | DAIVPQSFLKDDSIDNKVLTRSDKNRGKSDNVPSEEVVKKMKNYWRQLLNAKLITQRKFDNLTKAERG | |
| | GLSELDKAGFIKRQLVETRQITKHVAQILDSRMNTKYDENDKLIREVKVITLKSKLVSDFRKDFQFYK | |
| | VREINNYHHAHDAYLNAVVGTALIKKYPKLESEFVYGDYKVYDVRKMIAKSEQEIGKATAKYFFYSNI | |
| | MNFFKTEITLANGEIRKRPLIETNGETGEIVWDKGRDFATVRKVLSMPQVNIVKKTEVQTGGFSKESI | |
| | LPKRNSDKLIARKKDWDPKKYGGFDSPTVAYSVLVVAKVEKGKSKKLKSVKELLGITIMERSSFEKNP | |
| | IDFLEAKGYKEVKKDLIIKLPKYSLFELENGRKRMLASAGELQKGNELALPSKYVNFLYLASHYEKLK | |
| | GSPEDNEQKQLFVEQHKHYLDEIIEQISEFSKRVILADANLDKVLSAYNKHRDKPIREQAENIIHLFT | |
| | LTNLGAPAAFKYFDTTIDRKRYTSTKEVLDATLIHQSITGLYETRIDLSQLGGD | |
| ABE7.7 | MSEVEFSHEYWMRHALTLAKRAWDEREVPVGAVLVHNNRVIGEGWNRPIGRHDPTAHAEIMALRQGGL | 180 |
| | VMQNYRLIDATLYVTLEPCVMCAGAMIHSRIGRVVFGARDAKTGAAGSLMDVLHHPGMNHRVEITEGI | |
| | LADECAALLSDFFRMRRQEIKAQKKAQSSTDSGGSSGGSSGSETPGTSESATPESSGGSSGGSSEVEF | |
| | SHEYWMRHALTLAKRALDEREVPVGAVLVLNNRVIGEGWNRAIGLHDPTAHAEIMALRQGGLVMQNYR | |
| | LIDATLYVTFEPCVMCAGAMIHSRIGRVVFGVRNAKTGAAGSLMDVLHYPGMNHRVEITEGILADECA | |
| | ALLCYFFRMPRQVFNAQKKAQSSTDSGGSSGGSSGSETPGTSESATPESSGGSSGGSDKKYSIGLAIG | |
| | TNSVGWAVITDEYKVPSKKFKVLGNTDRHSIKKNLIGALLFDSGETAEATRLKRTARRRYTRRKNRIC | |
| | YLQEIFSNEMAKVDDSFFHRLEESFLVEEDKKHERHPIFGNIVDEVAYHEKYPTIYHLRKKLVDSTDK | |
| | ADLRLIYLALAHMIKFRGHFLIEGDLNPDNSDVDKLFIQLVQTYNQLFEENPINASGVDAKAILSARL | |
| | SKSRRLENLIAQLPGEKKNGLFGNLIALSLGLTPNFKSNFDLAEDAKLQLSKDTYDDDLDNLLAQIGD | |
| | QYADLFLAAKNLSDAILLSDILRVNTEITKAPLSASMIKRYDEHHQDLTLLKALVRQQLPEKYKEIFF | |
| | DQSKNGYAGYIDGGASQEEFYKFIKPILEKMDGTEELLVKLNREDLLRKQRTFDNGSIPHQIHLGELH | |
| | AILRRQEDFYPFLKDNREKIEKILTFRIPYYVGPLARGNSRFAWMTRKSEETITPWNFEEVVDKGASA | |
| | QSFIERMTNFDKNLPNEKVLPKHSLLYEYFTVYNELTKVKYVTEGMRKPAFLSGEQKKAIVDLLFKTN | |
| | RKVTVKQLKEDYFKKIECFDSVEISGVEDRFNASLGTYHDLLKIIKDKDFLDNEENEDILEDIVLTLT | |
| | LFEDREMIEERLKTYAHLFDDKVMKQLKRRRYTGWGRLSRKLINGIRDKQSGKTILDFLKSDGFANRN | |
| | FMQLIHDDSLTFKEDIQKAQVSGQGDSLHEHIANLAGSPAIKKGILQTVKVVDELVKVMGRHKPENIV | |
| | IEMARENQTTQKGQKNSRERMKRIEEGIKELGSQILKEHPVENTQLQNEKLYLYYLQNGRDMYVDQEL | |
| | DINRLSDYDVDHIVPQSFLKDDSIDNKVLTRSDKNRGKSDNVPSEEVVKKMKNYWRQLLNAKLITQRK | |
| | FDNLTKAERGGLSELDKAGFIKRQLVETRQITKHVAQILDSRMNTKYDENDKLIREVKVITLKSKLVS | |
| | DFRKDFQFYKVREINNYHHAHDAYLNAVVGTALIKKYPKLESEFVYGDYKVYDVRKMIAKSEQEIGKA | |
| | TAKYFFYSNIMNFFKTEITLANGEIRKRPLIETNGETGEIVWDKGRDFATVRKVLSMPQVNIVKKTEV | |
| | QTGGFSKESILPKRNSDKLIARKKDWDPKKYGGFDSPTVAYSVLVVAKVEKGKSKKLKSVKELLGITI | |
| | MERSSFEKNPIDFLEAKGYKEVKKDLIIKLPKYSLFELENGRKRMLASAGELQKGNELALPSKYVNFL | |
| | YLASHYEKLKGSPEDNEQKQLFVEQHKHYLDEIIEQISEFSKRVILADANLDKVLSAYNKHRDKPIRE | |
| | QAENIIHLFTLTNLGAPAAFKYFDTTIDRKRYTSTKEVLDATLIHQSITGLYETRIDLSQLGGDSGGS | |
| | PKKKRKV | |
| pNMG-624 | MSEVEFSHEYWMRHALTLAKRAWDEREVPVGAVLVHNNRVIGEGWNRPIGRHDPTAHAEIMALRQGGL | 181 |
| | VMQNYRLIDATLYVTLEPCVMCAGAMIHSRIGRVVFGARDAKTGAAGSLMDVLHHPGMNHRVEITEGI | |
| | LADECAALLSDFFRMRRQEIKAQKKAQSSTDSGGSSGGSSGSETPGTSESATPESSGGSSGGSSEVEF | |
| | SHEYWMRHALTLAKRARDEREVPVGAVLVLNNRVIGEGWNRAIGLHDPTAHAEIMALRQGGLVMQNYR | |
| | LIDATLYVTFEPCVMCAGAMIHSRIGRVVFGVRNAKTGAAGSLMDVLHYPGMNHRVEITEGILADECA | |
| | ALLCYFFRMPRQVFNAQKKAQSSTDSGGSSGGSSGSETPGTSESATPESDKKYSIGLAIGTNSVGWAV | |
| | ITDEYKVPSKKFKVLGNTDRHSIKKNLIGALLFDSGETAEATRLKRTARRRYTRRKNRICYLQEIFSN | |
| | EMAKVDDSFFHRLEESFLVEEDKKHERHPIFGNIVDEVAYHEKYPTIYHLRKKLVDSTDKADLRLIYL | |
| | ALAHMIKFRGHFLIEGDLNPDNSDVDKLFIQLVQTYNQLFEENPINASGVDAKAILSARLSKSRRLEN | |
| | LIAQLPGEKKNGLFGNLIALSLGLTPNFKSNFDLAEDAKLQLSKDTYDDDLDNLLAQIGDQYADLFLA | |
| | AKNLSDAILLSDILRVNTEITKAPLSASMIKRYDEHHQDLTLLKALVRQQLPEKYKEIFFDQSKNGYA | |
| | GYIDGGASQEEFYKFIKPILEKMDGTEELLVKLNREDLLRKQRTFDNGSIPHQIHLGELHAILRRQED | |
| | FYPFLKDNREKIEKILTFRIPYYVGPLARGNSRFAWMTRKSEETITPWNFEEVVDKGASAQSFIERMT | |
| | NFDKNLPNEKVLPKHSLLYEYFTVYNELTKVKYVTEGMRKPAFLSGEQKKAIVDLLFKTNRKVTVKQL | |
| | KEDYFKKIECFDSVEISGVEDRFNASLGTYHDLLKIIKDKDFLDNEENEDILEDIVLTLTLFEDREMI | |
| | EERLKTYAHLFDDKVMKQLKRRRYTGWGRLSRKLINGIRDKQSGKTILDFLKSDGFANRNFMQLIHDD | |
| | SLTFKEDIQKAQVSGQGDSLHEHIANLAGSPAIKKGILQTVKVVDELVKVMGRHKPENIVIEMARENQ | |
| | TTQKGQKNSRERMKRIEEGIKELGSQILKEHPVENTQLQNEKLYLYYLQNGRDMYVDQELDINRLSDY | |
| | DVDHIVPQSFLKDDSIDNKVLTRSDKNRGKSDNVPSEEVVKKMKNYWRQLLNAKLITQRKFDNLTKAE | |
| | RGGLSELDKAGFIKRQLVETRQITKHVAQILDSRMNTKYDENDKLIREVKVITLKSKLVSDFRKDFQF | |
| | YKVREINNYHHAHDAYLNAVVGTALIKKYPKLESEFVYGDYKVYDVRKMIAKSEQEIGKATAKYFFYS | |
| | NIMNFFKTEITLANGEIRKRPLIETNGETGEIVWDKGRDFATVRKVLSMPQVNIVKKTEVQTGGFSKE | |
| | SILPKRNSDKLIARKKDWDPKKYGGFDSPTVAYSVLVVAKVEKGKSKKLKSVKELLGITIMERSSFEK | |
| | NPIDFLEAKGYKEVKKDLIIKLPKYSLFELENGRKRMLASAGELQKGNELALPSKYVNFLYLASHYEK | |
| | LKGSPEDNEQKQLFVEQHKHYLDEIIEQISEFSKRVILADANLDKVLSAYNKHRDKPIREQAENIIHL | |
| | FTLTNLGAPAAFKYFDTTIDRKRYTSTKEVLDATLIHQSITGLYETRIDLSQLGGDSGGSPKKKRKV | |
| ABE3.2 | MSEVEFSHEYWMRHALTLAKRAWDEREVPVGAVLVHNNRVIGEGWNRPIGRHDPTAHAEIMALRQGGL | 182 |
| | VMQNYRLIDATLYVTLEPCVMCAGAMIHSRIGRVVFGARDAKTGAAGSLMDVLHHPGMNHRVEITEGI | |
| | LADECAALLSDFFRMRRQEIKAQKKAQSSTDSGGSSGGSSGSETPGTSESATPESSGGSSGGSSEVEF | |
| | SHEYWMRHALTLAKRAWDEREVPVGAVLVHNNRVIGEGWNRPIGRHDPTAHAEIMALRQGGLVMQNYR | |
| | LIDATLYVTFEPCVMCAGAMIHSRIGRVVFGVRNAKTGAAGSLMDVLHYPGMNHRVEITEGILADECA | |
| | ALLSYFFRMRRQVFKAQKKAQSSTDSGGSSGGSSGSETPGTSESATPESSGGSSGGSDKKYSIGLAIG | |
| | TNSVGWAVITDEYKVPSKKFKVLGNTDRHSIKKNLIGALLFDSGETAEATRLKRTARRRYTRRKNRIC | |
| | YLQEIFSNEMAKVDDSFFHRLEESFLVEEDKKHERHPIFGNIVDEVAYHEKYPTIYHLRKKLVDSTDK | |
| | ADLRLIYLALAHMIKFRGHFLIEGDLNPDNSDVDKLFIQLVQTYNQLFEENPINASGVDAKAILSARL | |
| | SKSRRLENLIAQLPGEKKNGLFGNLIALSLGLTPNFKSNFDLAEDAKLQLSKDTYDDDLDNLLAQIGD | |
| | QYADLFLAAKNLSDAILLSDILRVNTEITKAPLSASMIKRYDEHHQDLTLLKALVRQQLPEKYKEIFF | |
| | DQSKNGYAGYIDGGASQEEFYKFIKPILEKMDGTEELLVKLNREDLLRKQRTFDNGSIPHQIHLGELH | |
| | AILRRQEDFYPFLKDNREKIEKILTFRIPYYVGPLARGNSRFAWMTRKSEETITPWNFEEVVDKGASA | |
| | QSFIERMTNFDKNLPNEKVLPKHSLLYEYFTVYNELTKVKYVTEGMRKPAFLSGEQKKAIVDLLFKTN | |
| | RKVTVKQLKEDYFKKIECFDSVEISGVEDRFNASLGTYHDLLKIIKDKDFLDNEENEDILEDIVLTLT | |
| | LFEDREMIEERLKTYAHLFDDKVMKQLKRRRYTGWGRLSRKLINGIRDKQSGKTILDFLKSDGFANRN | |
| | FMQLIHDDSLTFKEDIQKAQVSGQGDSLHEHIANLAGSPAIKKGILQTVKVVDELVKVMGRHKPENIV | |
| | IEMARENQTTQKGQKNSRERMKRIEEGIKELGSQILKEHPVENTQLQNEKLYLYYLQNGRDMYVDQEL | |
| | DINRLSDYDVDHIVPQSFLKDDSIDNKVLTRSDKNRGKSDNVPSEEVVKKMKNYWRQLLNAKLITQRK | |
| | FDNLTKAERGGLSELDKAGFIKRQLVETRQITKHVAQILDSRMNTKYDENDKLIREVKVITLKSKLVS | |
| | DFRKDFQFYKVREINNYHHAHDAYLNAVVGTALIKKYPKLESEFVYGDYKVYDVRKMIAKSEQEIGKA | |
| | TAKYFFYSNIMNFFKTEITLANGEIRKRPLIETNGETGEIVWDKGRDFATVRKVLSMPQVNIVKKTEV | |
| | QTGGFSKESILPKRNSDKLIARKKDWDPKKYGGFDSPTVAYSVLVVAKVEKGKSKKLKSVKELLGITI | |
| | MERSSFEKNPIDFLEAKGYKEVKKDLIIKLPKYSLFELENGRKRMLASAGELQKGNELALPSKYVNFL | |
| | YLASHYEKLKGSPEDNEQKQLFVEQHKHYLDEIIEQISEFSKRVILADANLDKVLSAYNKHRDKPIRE | |
| | QAENIIHLFTLTNLGAPAAFKYFDTTIDRKRYTSTKEVLDATLIHQSITGLYETRIDLSQLGGDSGGS | |
| | PKKKRKV | |
| ABE5.3 | MSEVEFSHEYWMRHALTLAKRAWDEREVPVGAVLVHNNRVIGEGWNRPIGRHDPTAHAEIMALRQGGL | 183 |
| | VMQNYRLIDATLYVTLEPCVMCAGAMIHSRIGRVVFGARDAKTGAAGSLMDVLHHPGMNHRVEITEGI | |
| | LADECAALLSDFFRMRRQEIKAQKKAQSSTDSGGSSGGSSGSETPGTSESATPESSGGSSGGSSEVEF | |
| | SHEYWMRHALTLAKRAWDEREVPVGAVLVLNNRVIGEGWNRPIGLHDPTAHAEIMALRQGGLVMQNYR | |
| | LIDATLYVTFEPCVMCAGAMIHSRIGRVVFGVRNAKTGAAGSLMDVLHYPGMNHRVEITEGILADECA | |
| | ALLCYFFRMRRQVFNAQKKAQSSTDSGGSSGGSSGSETPGTSESATPESSGGSSGGSDKKYSIGLAIG | |
| | TNSVGWAVITDEYKVPSKKFKVLGNTDRHSIKKNLIGALLFDSGETAEATRLKRTARRRYTRRKNRIC | |
| | YLQEIFSNEMAKVDDSFFHRLEESFLVEEDKKHERHPIFGNIVDEVAYHEKYPTIYHLRKKLVDSTDK | |
| | ADLRLIYLALAHMIKFRGHFLIEGDLNPDNSDVDKLFIQLVQTYNQLFEENPINASGVDAKAILSARL | |
| | SKSRRLENLIAQLPGEKKNGLFGNLIALSLGLTPNFKSNFDLAEDAKLQLSKDTYDDDLDNLLAQIGD | |
| | QYADLFLAAKNLSDAILLSDILRVNTEITKAPLSASMIKRYDEHHQDLTLLKALVRQQLPEKYKEIFF | |
| | DQSKNGYAGYIDGGASQEEFYKFIKPILEKMDGTEELLVKLNREDLLRKQRTFDNGSIPHQIHLGELH | |
| | AILRRQEDFYPFLKDNREKIEKILTFRIPYYVGPLARGNSRFAWMTRKSEETITPWNFEEVVDKGASA | |
| | QSFIERMTNFDKNLPNEKVLPKHSLLYEYFTVYNELTKVKYVTEGMRKPAFLSGEQKKAIVDLLFKTN | |
| | RKVTVKQLKEDYFKKIECFDSVEISGVEDRFNASLGTYHDLLKIIKDKDFLDNEENEDILEDIVLTLT | |
| | LFEDREMIEERLKTYAHLFDDKVMKQLKRRRYTGWGRLSRKLINGIRDKQSGKTILDFLKSDGFANRN | |
| | FMQLIHDDSLTFKEDIQKAQVSGQGDSLHEHIANLAGSPAIKKGILQTVKVVDELVKVMGRHKPENIV | |
| | IEMARENQTTQKGQKNSRERMKRIEEGIKELGSQILKEHPVENTQLQNEKLYLYYLQNGRDMYVDQEL | |
| | DINRLSDYDVDHIVPQSFLKDDSIDNKVLTRSDKNRGKSDNVPSEEVVKKMKNYWRQLLNAKLITQRK | |
| | FDNLTKAERGGLSELDKAGFIKRQLVETRQITKHVAQILDSRMNTKYDENDKLIREVKVITLKSKLVS | |
| | DFRKDFQFYKVREINNYHHAHDAYLNAVVGTALIKKYPKLESEFVYGDYKVYDVRKMIAKSEQEIGKA | |
| | TAKYFFYSNIMNFFKTEITLANGEIRKRPLIETNGETGEIVWDKGRDFATVRKVLSMPQVNIVKKTEV | |
| | QTGGFSKESILPKRNSDKLIARKKDWDPKKYGGFDSPTVAYSVLVVAKVEKGKSKKLKSVKELLGITI | |
| | MERSSFEKNPIDFLEAKGYKEVKKDLIIKLPKYSLFELENGRKRMLASAGELQKGNELALPSKYVNFL | |
| | YLASHYEKLKGSPEDNEQKQLFVEQHKHYLDEIIEQISEFSKRVILADANLDKVLSAYNKHRDKPIRE | |
| | QAENIIHLFTLTNLGAPAAFKYFDTTIDRKRYTSTKEVLDATLIHQSITGLYETRIDLSQLGGDSGGS | |
| | PKKKRKV | |
| pNMG-558 | MSEVEFSHEYWMRHALTLAKRAWDEREVPVGAVLVHNNRVIGEGWNRPIGRHDPTAHAEIMALRQGGL | 184 |
| | VMQNYRLIDATLYVTLEPCVMCAGAMIHSRIGRVVFGARDAKTGAAGSLMDVLHHPGMNHRVEITEGI | |
| | LADECAALLSDFFRMRRQEIKAQKKAQSSTDSGGSSGGSSGSETPGTSESATPESSGGSSGGSSEVEF | |
| | SHEYWMRHALTLAKRAWDEREVPVGAVLVLNNRVIGEGWNRPIGLHDPTAHAEIMALRQGGLVMQNYR | |
| | LIDATLYVTFEPCVMCAGAMIHSRIGRVVFGVRNAKTGAAGSLMDVLHYPGMNHRVEITEGILADECA | |
| | ALLCYFFRMRRQVFNAQKKAQSSTDSGGSSGGSSGSETPGTSESATPESDKKYSIGLAIGTNSVGWAV | |
| | ITDEYKVPSKKFKVLGNTDRHSIKKNLIGALLFDSGETAEATRLKRTARRRYTRRKNRICYLQEIFSN | |
| | EMAKVDDSFFHRLEESFLVEEDKKHERHPIFGNIVDEVAYHEKYPTIYHLRKKLVDSTDKADLRLIYL | |
| | ALAHMIKFRGHFLIEGDLNPDNSDVDKLFIQLVQTYNQLFEENPINASGVDAKAILSARLSKSRRLEN | |
| | LIAQLPGEKKNGLFGNLIALSLGLTPNFKSNFDLAEDAKLQLSKDTYDDDLDNLLAQIGDQYADLFLA | |
| | AKNLSDAILLSDILRVNTEITKAPLSASMIKRYDEHHQDLTLLKALVRQQLPEKYKEIFFDQSKNGYA | |
| | GYIDGGASQEEFYKFIKPILEKMDGTEELLVKLNREDLLRKQRTFDNGSIPHQIHLGELHAILRRQED | |
| | FYPFLKDNREKIEKILTFRIPYYVGPLARGNSRFAWMTRKSEETITPWNFEEVVDKGASAQSFIERMT | |
| | NFDKNLPNEKVLPKHSLLYEYFTVYNELTKVKYVTEGMRKPAFLSGEQKKAIVDLLFKTNRKVTVKQL | |
| | KEDYFKKIECFDSVEISGVEDRFNASLGTYHDLLKIIKDKDFLDNEENEDILEDIVLTLTLFEDREMI | |
| | EERLKTYAHLFDDKVMKQLKRRRYTGWGRLSRKLINGIRDKQSGKTILDFLKSDGFANRNFMQLIHDD | |
| | SLTFKEDIQKAQVSGQGDSLHEHIANLAGSPAIKKGILQTVKVVDELVKVMGRHKPENIVIEMARENQ | |
| | TTQKGQKNSRERMKRIEEGIKELGSQILKEHPVENTQLQNEKLYLYYLQNGRDMYVDQELDINRLSDY | |
| | DVDHIVPQSFLKDDSIDNKVLTRSDKNRGKSDNVPSEEVVKKMKNYWRQLLNAKLITQRKFDNLTKAE | |
| | RGGLSELDKAGFIKRQLVETRQITKHVAQILDSRMNTKYDENDKLIREVKVITLKSKLVSDFRKDFQF | |
| | YKVREINNYHHAHDAYLNAVVGTALIKKYPKLESEFVYGDYKVYDVRKMIAKSEQEIGKATAKYFFYS | |
| | NIMNFFKTEITLANGEIRKRPLIETNGETGEIVWDKGRDFATVRKVLSMPQVNIVKKTEVQTGGFSKE | |
| | SILPKRNSDKLIARKKDWDPKKYGGFDSPTVAYSVLVVAKVEKGKSKKLKSVKELLGITIMERSSFEK | |
| | NPIDFLEAKGYKEVKKDLIIKLPKYSLFELENGRKRMLASAGELQKGNELALPSKYVNFLYLASHYEK | |
| | LKGSPEDNEQKQLFVEQHKHYLDEIIEQISEFSKRVILADANLDKVLSAYNKHRDKPIREQAENIIHL | |
| | FTLTNLGAPAAFKYFDTTIDRKRYTSTKEVLDATLIHQSITGLYETRIDLSQLGGDSGGSPKKKRKV | |
| pNMG-576 | MSEVEFSHEYWMRHALTLAKRAWDEREVPVGAVLVHNNRVIGEGWNRPIGRHDPTAHAEIMALRQGGL | 185 |
| | VMQNYRLIDATLYVTLEPCVMCAGAMIHSRIGRVVFGARDAKTGAAGSLMDVLHHPGMNHRVEITEGI | |
| | LADECAALLSDFFRMRRQEIKAQKKAQSSTDSGGSSGGSSGSETPGTSESATPESSGGSSGGSSEVEF | |
| | SHEYWMRHALTLAKRAWDEREVPVGAVLVLNNRVIGEGWNRSIGLHDPTAHAEIMALRQGGLVMQNYR | |
| | LIDATLYVTFEPCVMCAGAMIHSRIGRVVFGVRNAKTGAAGSLMDVLHYPGMNHRVEITEGILADECA | |
| | ALLCYFFRMRRQVFNAQKKAQSSTDSGGSSGGSSGSETPGTSESATPESSGGSSGGSDKKYSIGLAIG | |
| | TNSVGWAVITDEYKVPSKKFKVLGNTDRHSIKKNLIGALLFDSGETAEATRLKRTARRRYTRRKNRIC | |
| | YLQEIFSNEMAKVDDSFFHRLEESFLVEEDKKHERHPIFGNIVDEVAYHEKYPTIYHLRKKLVDSTDK | |
| | ADLRLIYLALAHMIKFRGHFLIEGDLNPDNSDVDKLFIQLVQTYNQLFEENPINASGVDAKAILSARL | |
| | SKSRRLENLIAQLPGEKKNGLFGNLIALSLGLTPNFKSNFDLAEDAKLQLSKDTYDDDLDNLLAQIGD | |
| | QYADLFLAAKNLSDAILLSDILRVNTEITKAPLSASMIKRYDEHHQDLTLLKALVRQQLPEKYKEIFF | |
| | DQSKNGYAGYIDGGASQEEFYKFIKPILEKMDGTEELLVKLNREDLLRKQRTFDNGSIPHQIHLGELH | |
| | AILRRQEDFYPFLKDNREKIEKILTFRIPYYVGPLARGNSRFAWMTRKSEETITPWNFEEVVDKGASA | |
| | QSFIERMTNFDKNLPNEKVLPKHSLLYEYFTVYNELTKVKYVTEGMRKPAFLSGEQKKAIVDLLFKTN | |
| | RKVTVKQLKEDYFKKIECFDSVEISGVEDRFNASLGTYHDLLKIIKDKDFLDNEENEDILEDIVLTLT | |
| | LFEDREMIEERLKTYAHLFDDKVMKQLKRRRYTGWGRLSRKLINGIRDKQSGKTILDFLKSDGFANRN | |
| | FMQLIHDDSLTFKEDIQKAQVSGQGDSLHEHIANLAGSPAIKKGILQTVKVVDELVKVMGRHKPENIV | |
| | IEMARENQTTQKGQKNSRERMKRIEEGIKELGSQILKEHPVENTQLQNEKLYLYYLQNGRDMYVDQEL | |
| | DINRLSDYDVDHIVPQSFLKDDSIDNKVLTRSDKNRGKSDNVPSEEVVKKMKNYWRQLLNAKLITQRK | |
| | FDNLTKAERGGLSELDKAGFIKRQLVETRQITKHVAQILDSRMNTKYDENDKLIREVKVITLKSKLVS | |
| | DFRKDFQFYKVREINNYHHAHDAYLNAVVGTALIKKYPKLESEFVYGDYKVYDVRKMIAKSEQEIGKA | |
| | TAKYFFYSNIMNFFKTEITLANGEIRKRPLIETNGETGEIVWDKGRDFATVRKVLSMPQVNIVKKTEV | |
| | QTGGFSKESILPKRNSDKLIARKKDWDPKKYGGFDSPTVAYSVLVVAKVEKGKSKKLKSVKELLGITI | |
| | MERSSFEKNPIDFLEAKGYKEVKKDLIIKLPKYSLFELENGRKRMLASAGELQKGNELALPSKYVNFL | |
| | YLASHYEKLKGSPEDNEQKQLFVEQHKHYLDEIIEQISEFSKRVILADANLDKVLSAYNKHRDKPIRE | |
| | QAENIIHLFTLTNLGAPAAFKYFDTTIDRKRYTSTKEVLDATLIHQSITGLYETRIDLSQLGGDSGGS | |
| | PKKKRKV | |
| pNMG-577 | MSEVEFSHEYWMRHALTLAKRAWDEREVPVGAVLVHNNRVIGEGWNRPIGRHDPTAHAEIMALRQGGL | 186 |
| | VMQNYRLIDATLYVTLEPCVMCAGAMIHSRIGRVVFGARDAKTGAAGSLMDVLHHPGMNHRVEITEGI | |
| | LADECAALLSDFFRMRRQEIKAQKKAQSSTDSGGSSGGSSGSETPGTSESATPESSGGSSGGSSEVEF | |
| | SHEYWMRHALTLAKRAWDEREVPVGAVLVLNNRVIGEGWNRSIGLHDPTAHAEIMALRQGGLVMQNYR | |
| | LIDATLYVTFEPCVMCAGAMIHSRIGRVVFGVRNAKTGAAGSLMDVLHYPGMNHRVEITEGILADECN | |
| | ALLCYFFRMRRQVFNAQKKAQSSTDSGGSSGGSSGSETPGTSESATPESSGGSSGGSDKKYSIGLAIG | |
| | TNSVGWAVITDEYKVPSKKFKVLGNTDRHSIKKNLIGALLFDSGETAEATRLKRTARRRYTRRKNRIC | |
| | YLQEIFSNEMAKVDDSFFHRLEESFLVEEDKKHERHPIFGNIVDEVAYHEKYPTIYHLRKKLVDSTDK | |
| | ADLRLIYLALAHMIKFRGHFLIEGDLNPDNSDVDKLFIQLVQTYNQLFEENPINASGVDAKAILSARL | |
| | SKSRRLENLIAQLPGEKKNGLFGNLIALSLGLTPNFKSNFDLAEDAKLQLSKDTYDDDLDNLLAQIGD | |
| | QYADLFLAAKNLSDAILLSDILRVNTEITKAPLSASMIKRYDEHHQDLTLLKALVRQQLPEKYKEIFF | |
| | DQSKNGYAGYIDGGASQEEFYKFIKPILEKMDGTEELLVKLNREDLLRKQRTFDNGSIPHQIHLGELH | |
| | AILRRQEDFYPFLKDNREKIEKILTFRIPYYVGPLARGNSRFAWMTRKSEETITPWNFEEVVDKGASA | |
| | QSFIERMTNFDKNLPNEKVLPKHSLLYEYFTVYNELTKVKYVTEGMRKPAFLSGEQKKAIVDLLFKTN | |
| | RKVTVKQLKEDYFKKIECFDSVEISGVEDRFNASLGTYHDLLKIIKDKDFLDNEENEDILEDIVLTLT | |
| | LFEDREMIEERLKTYAHLFDDKVMKQLKRRRYTGWGRLSRKLINGIRDKQSGKTILDFLKSDGFANRN | |
| | FMQLIHDDSLTFKEDIQKAQVSGQGDSLHEHIANLAGSPAIKKGILQTVKVVDELVKVMGRHKPENIV | |
| | IEMARENQTTQKGQKNSRERMKRIEEGIKELGSQILKEHPVENTQLQNEKLYLYYLQNGRDMYVDQEL | |
| | DINRLSDYDVDHIVPQSFLKDDSIDNKVLTRSDKNRGKSDNVPSEEVVKKMKNYWRQLLNAKLITQRK | |
| | FDNLTKAERGGLSELDKAGFIKRQLVETRQITKHVAQILDSRMNTKYDENDKLIREVKVITLKSKLVS | |
| | DFRKDFQFYKVREINNYHHAHDAYLNAVVGTALIKKYPKLESEFVYGDYKVYDVRKMIAKSEQEIGKA | |
| | TAKYFFYSNIMNFFKTEITLANGEIRKRPLIETNGETGEIVWDKGRDFATVRKVLSMPQVNIVKKTEV | |
| | QTGGFSKESILPKRNSDKLIARKKDWDPKKYGGFDSPTVAYSVLVVAKVEKGKSKKLKSVKELLGITI | |
| | MERSSFEKNPIDFLEAKGYKEVKKDLIIKLPKYSLFELENGRKRMLASAGELQKGNELALPSKYVNFL | |
| | YLASHYEKLKGSPEDNEQKQLFVEQHKHYLDEIIEQISEFSKRVILADANLDKVLSAYNKHRDKPIRE | |
| | QAENIIHLFTLTNLGAPAAFKYFDTTIDRKRYTSTKEVLDATLIHQSITGLYETRIDLSQLGGDSGGS | |
| | PKKKRKV | |
| pNMG-586 | MSEVEFSHEYWMRHALTLAKRAWDEREVPVGAVLVHNNRVIGEGWNRPIGRHDPTAHAEIMALRQGGL | 187 |
| | VMQNYRLIDATLYVTLEPCVMCAGAMIHSRIGRVVFGARDAKTGAAGSLMDVLHHPGMNHRVEITEGI | |
| | LADECAALLSDFFRMRRQEIKAQKKAQSSTDSGGSSGGSSGSETPGTSESATPESSGGSSGGSSEVEF | |
| | SHEYWMRHALTLAKRAWDEREVPVGAVLVLNNRVIGEGWNRAIGLHDPTAHAEIMALRQGGLVMQNYR | |
| | LIDATLYVTFEPCVMCAGAMIHSRIGRVVFGVRNAKTGAAGSLMDVLHYPGMNHRVEITEGILADECA | |
| | ALLCYFFRMRRQVFNAQKKAQSSTDSGGSSGGSSGSETPGTSESATPESSGGSSGGSDKKYSIGLAIG | |
| | TNSVGWAVITDEYKVPSKKFKVLGNTDRHSIKKNLIGALLFDSGETAEATRLKRTARRRYTRRKNRIC | |
| | YLQEIFSNEMAKVDDSFFHRLEESFLVEEDKKHERHPIFGNIVDEVAYHEKYPTIYHLRKKLVDSTDK | |
| | ADLRLIYLALAHMIKFRGHFLIEGDLNPDNSDVDKLFIQLVQTYNQLFEENPINASGVDAKAILSARL | |
| | SKSRRLENLIAQLPGEKKNGLFGNLIALSLGLTPNFKSNFDLAEDAKLQLSKDTYDDDLDNLLAQIGD | |
| | QYADLFLAAKNLSDAILLSDILRVNTEITKAPLSASMIKRYDEHHQDLTLLKALVRQQLPEKYKEIFF | |
| | DQSKNGYAGYIDGGASQEEFYKFIKPILEKMDGTEELLVKLNREDLLRKQRTFDNGSIPHQIHLGELH | |
| | AILRRQEDFYPFLKDNREKIEKILTFRIPYYVGPLARGNSRFAWMTRKSEETITPWNFEEVVDKGASA | |
| | QSFIERMTNFDKNLPNEKVLPKHSLLYEYFTVYNELTKVKYVTEGMRKPAFLSGEQKKAIVDLLFKTN | |
| | RKVTVKQLKEDYFKKIECFDSVEISGVEDRFNASLGTYHDLLKIIKDKDFLDNEENEDILEDIVLTLT | |
| | LFEDREMIEERLKTYAHLFDDKVMKQLKRRRYTGWGRLSRKLINGIRDKQSGKTILDFLKSDGFANRN | |
| | FMQLIHDDSLTFKEDIQKAQVSGQGDSLHEHIANLAGSPAIKKGILQTVKVVDELVKVMGRHKPENIV | |
| | IEMARENQTTQKGQKNSRERMKRIEEGIKELGSQILKEHPVENTQLQNEKLYLYYLQNGRDMYVDQEL | |
| | DINRLSDYDVDHIVPQSFLKDDSIDNKVLTRSDKNRGKSDNVPSEEVVKKMKNYWRQLLNAKLITQRK | |
| | FDNLTKAERGGLSELDKAGFIKRQLVETRQITKHVAQILDSRMNTKYDENDKLIREVKVITLKSKLVS | |
| | DFRKDFQFYKVREINNYHHAHDAYLNAVVGTALIKKYPKLESEFVYGDYKVYDVRKMIAKSEQEIGKA | |
| | TAKYFFYSNIMNFFKTEITLANGEIRKRPLIETNGETGEIVWDKGRDFATVRKVLSMPQVNIVKKTEV | |
| | QTGGFSKESILPKRNSDKLIARKKDWDPKKYGGFDSPTVAYSVLVVAKVEKGKSKKLKSVKELLGITI | |
| | MERSSFEKNPIDFLEAKGYKEVKKDLIIKLPKYSLFELENGRKRMLASAGELQKGNELALPSKYVNFL | |
| | YLASHYEKLKGSPEDNEQKQLFVEQHKHYLDEIIEQISEFSKRVILADANLDKVLSAYNKHRDKPIRE | |
| | QAENIIHLFTLTNLGAPAAFKYFDTTIDRKRYTSTKEVLDATLIHQSITGLYETRIDLSQLGGDSGGS | |
| | PKKKRKV | |
| ABE7.2 | MSEVEFSHEYWMRHALTLAKRAWDEREVPVGAVLVHNNRVIGEGWNRPIGRHDPTAHAEIMALRQGGL | 188 |
| | VMQNYRLIDATLYVTLEPCVMCAGAMIHSRIGRVVFGARDAKTGAAGSLMDVLHHPGMNHRVEITEGI | |
| | LADECAALLSDFFRMRRQEIKAQKKAQSSTDSGGSSGGSSGSETPGTSESATPESSGGSSGGSSEVEF | |
| | SHEYWMRHALTLAKRAWDEREVPVGAVLVLNNRVIGEGWNRAIGLHDPTAHAEIMALRQGGLVMQNYR | |
| | LIDATLYVTFEPCVMCAGAMIHSRIGRVVFGVRNAKTGAAGSLMDVLHYPGMNHRVEITEGILADECN | |
| | ALLCYFFRMRRQVFNAQKKAQSSTDSGGSSGGSSGSETPGTSESATPESSGGSSGGSDKKYSIGLAIG | |
| | TNSVGWAVITDEYKVPSKKFKVLGNTDRHSIKKNLIGALLFDSGETAEATRLKRTARRRYTRRKNRIC | |
| | YLQEIFSNEMAKVDDSFFHRLEESFLVEEDKKHERHPIFGNIVDEVAYHEKYPTIYHLRKKLVDSTDK | |
| | ADLRLIYLALAHMIKFRGHFLIEGDLNPDNSDVDKLFIQLVQTYNQLFEENPINASGVDAKAILSARL | |
| | SKSRRLENLIAQLPGEKKNGLFGNLIALSLGLTPNFKSNFDLAEDAKLQLSKDTYDDDLDNLLAQIGD | |
| | QYADLFLAAKNLSDAILLSDILRVNTEITKAPLSASMIKRYDEHHQDLTLLKALVRQQLPEKYKEIFF | |
| | DQSKNGYAGYIDGGASQEEFYKFIKPILEKMDGTEELLVKLNREDLLRKQRTFDNGSIPHQIHLGELH | |
| | AILRRQEDFYPFLKDNREKIEKILTFRIPYYVGPLARGNSRFAWMTRKSEETITPWNFEEVVDKGASA | |
| | QSFIERMTNFDKNLPNEKVLPKHSLLYEYFTVYNELTKVKYVTEGMRKPAFLSGEQKKAIVDLLFKTN | |
| | RKVTVKQLKEDYFKKIECFDSVEISGVEDRFNASLGTYHDLLKIIKDKDFLDNEENEDILEDIVLTLT | |
| | LFEDREMIEERLKTYAHLFDDKVMKQLKRRRYTGWGRLSRKLINGIRDKQSGKTILDFLKSDGFANRN | |
| | FMQLIHDDSLTFKEDIQKAQVSGQGDSLHEHIANLAGSPAIKKGILQTVKVVDELVKVMGRHKPENIV | |
| | IEMARENQTTQKGQKNSRERMKRIEEGIKELGSQILKEHPVENTQLQNEKLYLYYLQNGRDMYVDQEL | |
| | DINRLSDYDVDHIVPQSFLKDDSIDNKVLTRSDKNRGKSDNVPSEEVVKKMKNYWRQLLNAKLITQRK | |
| | FDNLTKAERGGLSELDKAGFIKRQLVETRQITKHVAQILDSRMNTKYDENDKLIREVKVITLKSKLVS | |
| | DFRKDFQFYKVREINNYHHAHDAYLNAVVGTALIKKYPKLESEFVYGDYKVYDVRKMIAKSEQEIGKA | |
| | TAKYFFYSNIMNFFKTEITLANGEIRKRPLIETNGETGEIVWDKGRDFATVRKVLSMPQVNIVKKTEV | |
| | QTGGFSKESILPKRNSDKLIARKKDWDPKKYGGFDSPTVAYSVLVVAKVEKGKSKKLKSVKELLGITI | |
| | MERSSFEKNPIDFLEAKGYKEVKKDLIIKLPKYSLFELENGRKRMLASAGELQKGNELALPSKYVNFL | |
| | YLASHYEKLKGSPEDNEQKQLFVEQHKHYLDEIIEQISEFSKRVILADANLDKVLSAYNKHRDKPIRE | |
| | QAENIIHLFTLTNLGAPAAFKYFDTTIDRKRYTSTKEVLDATLIHQSITGLYETRIDLSQLGGDSGGS | |
| | PKKKRKV | |
| pNMG-620 | MSEVEFSHEYWMRHALTLAKRAWDEREVPVGAVLVHNNRVIGEGWNRPIGRHDPTAHAEIMALRQGGL | 189 |
| | VMQNYRLIDATLYVTLEPCVMCAGAMIHSRIGRVVFGARDAKTGAAGSLMDVLHHPGMNHRVEITEGI | |
| | LADECAALLSDFFRMRRQEIKAQKKAQSSTDSGGSSGGSSGSETPGTSESATPESSGGSSGGSSEVEF | |
| | SHEYWMRHALTLAKRARDEREVPVGAVLVLNNRVIGEGWNRAIGLHDPTAHAEIMALRQGGLVMQNYR | |
| | LIDATLYVTFEPCVMCAGAMIHSRIGRVVFGVRNAKTGAAGSLMDVLHYPGMNHRVEITEGILADECA | |
| | ALLCYFFRMPRQVFNAQKKAQSSTDSGGSSGGSSGSETPGTSESATPESSGGSSGGSDKKYSIGLAIG | |
| | TNSVGWAVITDEYKVPSKKFKVLGNTDRHSIKKNLIGALLFDSGETAEATRLKRTARRRYTRRKNRIC | |
| | YLQEIFSNEMAKVDDSFFHRLEESFLVEEDKKHERHPIFGNIVDEVAYHEKYPTIYHLRKKLVDSTDK | |
| | ADLRLIYLALAHMIKFRGHFLIEGDLNPDNSDVDKLFIQLVQTYNQLFEENPINASGVDAKAILSARL | |
| | SKSRRLENLIAQLPGEKKNGLFGNLIALSLGLTPNFKSNFDLAEDAKLQLSKDTYDDDLDNLLAQIGD | |
| | QYADLFLAAKNLSDAILLSDILRVNTEITKAPLSASMIKRYDEHHQDLTLLKALVRQQLPEKYKEIFF | |
| | DQSKNGYAGYIDGGASQEEFYKFIKPILEKMDGTEELLVKLNREDLLRKQRTFDNGSIPHQIHLGELH | |
| | AILRRQEDFYPFLKDNREKIEKILTFRIPYYVGPLARGNSRFAWMTRKSEETITPWNFEEVVDKGASA | |
| | QSFIERMTNFDKNLPNEKVLPKHSLLYEYFTVYNELTKVKYVTEGMRKPAFLSGEQKKAIVDLLFKTN | |
| | RKVTVKQLKEDYFKKIECFDSVEISGVEDRFNASLGTYHDLLKIIKDKDFLDNEENEDILEDIVLTLT | |
| | LFEDREMIEERLKTYAHLFDDKVMKQLKRRRYTGWGRLSRKLINGIRDKQSGKTILDFLKSDGFANRN | |
| | FMQLIHDDSLTFKEDIQKAQVSGQGDSLHEHIANLAGSPAIKKGILQTVKVVDELVKVMGRHKPENIV | |
| | IEMARENQTTQKGQKNSRERMKRIEEGIKELGSQILKEHPVENTQLQNEKLYLYYLQNGRDMYVDQEL | |
| | DINRLSDYDVDHIVPQSFLKDDSIDNKVLTRSDKNRGKSDNVPSEEVVKKMKNYWRQLLNAKLITQRK | |
| | FDNLTKAERGGLSELDKAGFIKRQLVETRQITKHVAQILDSRMNTKYDENDKLIREVKVITLKSKLVS | |
| | DFRKDFQFYKVREINNYHHAHDAYLNAVVGTALIKKYPKLESEFVYGDYKVYDVRKMIAKSEQEIGKA | |
| | TAKYFFYSNIMNFFKTEITLANGEIRKRPLIETNGETGEIVWDKGRDFATVRKVLSMPQVNIVKKTEV | |
| | QTGGFSKESILPKRNSDKLIARKKDWDPKKYGGFDSPTVAYSVLVVAKVEKGKSKKLKSVKELLGITI | |
| | MERSSFEKNPIDFLEAKGYKEVKKDLIIKLPKYSLFELENGRKRMLASAGELQKGNELALPSKYVNFL | |
| | YLASHYEKLKGSPEDNEQKQLFVEQHKHYLDEIIEQISEFSKRVILADANLDKVLSAYNKHRDKPIRE | |
| | QAENIIHLFTLTNLGAPAAFKYFDTTIDRKRYTSTKEVLDATLIHQSITGLYETRIDLSQLGGDSGGS | |
| | PKKKRKV | |
| pNMG-617 | MSEVEFSHEYWMRHALTLAKRAWDEREVPVGAVLVHNNRVIGEGWNRPIGRHDPTAHAEIMALRQGGL | 190 |
| | VMQNYRLIDATLYVTLEPCVMCAGAMIHSRIGRVVFGARDAKTGAAGSLMDVLHHPGMNHRVEITEGI | |
| | LADECAALLSDFFRMRRQEIKAQKKAQSSTDSGGSSGGSSGSETPGTSESATPESSGGSSGGSSEVEF | |
| | SHEYWMRHALTLAKRALDEREVPVGAVLVLNNRVIGEGWNRAIGLHDPTAHAEIMALRQGGLVMQNYR | |
| | LIDATLYVTFEPCVMCAGAMIHSRIGRVVFGVRNAKTGAAGSLMDVLHYPGMNHRVEITEGILADECN | |
| | ALLCYFFRMRRQVFNAQKKAQSSTDSGGSSGGSSGSETPGTSESATPESSGGSSGGSDKKYSIGLAIG | |
| | TNSVGWAVITDEYKVPSKKFKVLGNTDRHSIKKNLIGALLFDSGETAEATRLKRTARRRYTRRKNRIC | |
| | YLQEIFSNEMAKVDDSFFHRLEESFLVEEDKKHERHPIFGNIVDEVAYHEKYPTIYHLRKKLVDSTDK | |
| | ADLRLIYLALAHMIKFRGHFLIEGDLNPDNSDVDKLFIQLVQTYNQLFEENPINASGVDAKAILSARL | |
| | SKSRRLENLIAQLPGEKKNGLFGNLIALSLGLTPNFKSNFDLAEDAKLQLSKDTYDDDLDNLLAQIGD | |
| | QYADLFLAAKNLSDAILLSDILRVNTEITKAPLSASMIKRYDEHHQDLTLLKALVRQQLPEKYKEIFF | |
| | DQSKNGYAGYIDGGASQEEFYKFIKPILEKMDGTEELLVKLNREDLLRKQRTFDNGSIPHQIHLGELH | |
| | AILRRQEDFYPFLKDNREKIEKILTFRIPYYVGPLARGNSRFAWMTRKSEETITPWNFEEVVDKGASA | |
| | QSFIERMTNFDKNLPNEKVLPKHSLLYEYFTVYNELTKVKYVTEGMRKPAFLSGEQKKAIVDLLFKTN | |
| | RKVTVKQLKEDYFKKIECFDSVEISGVEDRFNASLGTYHDLLKIIKDKDFLDNEENEDILEDIVLTLT | |
| | LFEDREMIEERLKTYAHLFDDKVMKQLKRRRYTGWGRLSRKLINGIRDKQSGKTILDFLKSDGFANRN | |
| | FMQLIHDDSLTFKEDIQKAQVSGQGDSLHEHIANLAGSPAIKKGILQTVKVVDELVKVMGRHKPENIV | |
| | IEMARENQTTQKGQKNSRERMKRIEEGIKELGSQILKEHPVENTQLQNEKLYLYYLQNGRDMYVDQEL | |
| | DINRLSDYDVDHIVPQSFLKDDSIDNKVLTRSDKNRGKSDNVPSEEVVKKMKNYWRQLLNAKLITQRK | |
| | FDNLTKAERGGLSELDKAGFIKRQLVETRQITKHVAQILDSRMNTKYDENDKLIREVKVITLKSKLVS | |
| | DFRKDFQFYKVREINNYHHAHDAYLNAVVGTALIKKYPKLESEFVYGDYKVYDVRKMIAKSEQEIGKA | |
| | TAKYFFYSNIMNFFKTEITLANGEIRKRPLIETNGETGEIVWDKGRDFATVRKVLSMPQVNIVKKTEV | |
| | QTGGFSKESILPKRNSDKLIARKKDWDPKKYGGFDSPTVAYSVLVVAKVEKGKSKKLKSVKELLGITI | |
| | MERSSFEKNPIDFLEAKGYKEVKKDLIIKLPKYSLFELENGRKRMLASAGELQKGNELALPSKYVNFL | |
| | YLASHYEKLKGSPEDNEQKQLFVEQHKHYLDEIIEQISEFSKRVILADANLDKVLSAYNKHRDKPIRE | |
| | QAENIIHLFTLTNLGAPAAFKYFDTTIDRKRYTSTKEVLDATLIHQSITGLYETRIDLSQLGGDSGGS | |
| | PKKKRKV | |
| pNMG-618 | MSEVEFSHEYWMRHALTLAKRAWDEREVPVGAVLVHNNRVIGEGWNRPIGRHDPTAHAEIMALRQGGL | 191 |
| | VMQNYRLIDATLYVTLEPCVMCAGAMIHSRIGRVVFGARDAKTGAAGSLMDVLHHPGMNHRVEITEGI | |
| | LADECAALLSDFFRMRRQEIKAQKKAQSSTDSGGSSGGSSGSETPGTSESATPESSGGSSGGSSEVEF | |
| | SHEYWMRHALTLAKRALDEREVPVGAVLVLNNRVIGEGWNRAIGLHDPTAHAEIMALRQGGLVMQNYR | |
| | LIDATLYVTFEPCVMCAGAMIHSRIGRVVFGVRNAKTGAAGSLMDVLHYPGMNHRVEITEGILADECN | |
| | ALLCYFFRMPRQVFNAQKKAQSSTDSGGSSGGSSGSETPGTSESATPESSGGSSGGSDKKYSIGLAIG | |
| | TNSVGWAVITDEYKVPSKKFKVLGNTDRHSIKKNLIGALLFDSGETAEATRLKRTARRRYTRRKNRIC | |
| | YLQEIFSNEMAKVDDSFFHRLEESFLVEEDKKHERHPIFGNIVDEVAYHEKYPTIYHLRKKLVDSTDK | |
| | ADLRLIYLALAHMIKFRGHFLIEGDLNPDNSDVDKLFIQLVQTYNQLFEENPINASGVDAKAILSARL | |
| | SKSRRLENLIAQLPGEKKNGLFGNLIALSLGLTPNFKSNFDLAEDAKLQLSKDTYDDDLDNLLAQIGD | |
| | QYADLFLAAKNLSDAILLSDILRVNTEITKAPLSASMIKRYDEHHQDLTLLKALVRQQLPEKYKEIFF | |
| | DQSKNGYAGYIDGGASQEEFYKFIKPILEKMDGTEELLVKLNREDLLRKQRTFDNGSIPHQIHLGELH | |
| | AILRRQEDFYPFLKDNREKIEKILTFRIPYYVGPLARGNSRFAWMTRKSEETITPWNFEEVVDKGASA | |
| | QSFIERMTNFDKNLPNEKVLPKHSLLYEYFTVYNELTKVKYVTEGMRKPAFLSGEQKKAIVDLLFKTN | |
| | RKVTVKQLKEDYFKKIECFDSVEISGVEDRFNASLGTYHDLLKIIKDKDFLDNEENEDILEDIVLTLT | |
| | LFEDREMIEERLKTYAHLFDDKVMKQLKRRRYTGWGRLSRKLINGIRDKQSGKTILDFLKSDGFANRN | |
| | FMQLIHDDSLTFKEDIQKAQVSGQGDSLHEHIANLAGSPAIKKGILQTVKVVDELVKVMGRHKPENIV | |
| | IEMARENQTTQKGQKNSRERMKRIEEGIKELGSQILKEHPVENTQLQNEKLYLYYLQNGRDMYVDQEL | |
| | DINRLSDYDVDHIVPQSFLKDDSIDNKVLTRSDKNRGKSDNVPSEEVVKKMKNYWRQLLNAKLITQRK | |
| | FDNLTKAERGGLSELDKAGFIKRQLVETRQITKHVAQILDSRMNTKYDENDKLIREVKVITLKSKLVS | |
| | DFRKDFQFYKVREINNYHHAHDAYLNAVVGTALIKKYPKLESEFVYGDYKVYDVRKMIAKSEQEIGKA | |
| | TAKYFFYSNIMNFFKTEITLANGEIRKRPLIETNGETGEIVWDKGRDFATVRKVLSMPQVNIVKKTEV | |
| | QTGGFSKESILPKRNSDKLIARKKDWDPKKYGGFDSPTVAYSVLVVAKVEKGKSKKLKSVKELLGITI | |
| | MERSSFEKNPIDFLEAKGYKEVKKDLIIKLPKYSLFELENGRKRMLASAGELQKGNELALPSKYVNFL | |
| | YLASHYEKLKGSPEDNEQKQLFVEQHKHYLDEIIEQISEFSKRVILADANLDKVLSAYNKHRDKPIRE | |
| | QAENIIHLFTLTNLGAPAAFKYFDTTIDRKRYTSTKEVLDATLIHQSITGLYETRIDLSQLGGDSGGS | |
| | PKKKRKV | |
| pNMG-620 | MSEVEFSHEYWMRHALTLAKRAWDEREVPVGAVLVHNNRVIGEGWNRPIGRHDPTAHAEIMALRQGGL | 192 |
| | VMQNYRLIDATLYVTLEPCVMCAGAMIHSRIGRVVFGARDAKTGAAGSLMDVLHHPGMNHRVEITEGI | |
| | LADECAALLSDFFRMRRQEIKAQKKAQSSTDSGGSSGGSSGSETPGTSESATPESSGGSSGGSSEVEF | |
| | SHEYWMRHALTLAKRARDEREVPVGAVLVLNNRVIGEGWNRAIGLHDPTAHAEIMALRQGGLVMQNYR | |
| | LIDATLYVTFEPCVMCAGAMIHSRIGRVVFGVRNAKTGAAGSLMDVLHYPGMNHRVEITEGILADECA | |
| | ALLCYFFRMPRQVFNAQKKAQSSTDSGGSSGGSSGSETPGTSESATPESSGGSSGGSDKKYSIGLAIG | |
| | TNSVGWAVITDEYKVPSKKFKVLGNTDRHSIKKNLIGALLFDSGETAEATRLKRTARRRYTRRKNRIC | |
| | YLQEIFSNEMAKVDDSFFHRLEESFLVEEDKKHERHPIFGNIVDEVAYHEKYPTIYHLRKKLVDSTDK | |
| | ADLRLIYLALAHMIKFRGHFLIEGDLNPDNSDVDKLFIQLVQTYNQLFEENPINASGVDAKAILSARL | |
| | SKSRRLENLIAQLPGEKKNGLFGNLIALSLGLTPNFKSNFDLAEDAKLQLSKDTYDDDLDNLLAQIGD | |
| | QYADLFLAAKNLSDAILLSDILRVNTEITKAPLSASMIKRYDEHHQDLTLLKALVRQQLPEKYKEIFF | |
| | DQSKNGYAGYIDGGASQEEFYKFIKPILEKMDGTEELLVKLNREDLLRKQRTFDNGSIPHQIHLGELH | |
| | AILRRQEDFYPFLKDNREKIEKILTFRIPYYVGPLARGNSRFAWMTRKSEETITPWNFEEVVDKGASA | |
| | QSFIERMTNFDKNLPNEKVLPKHSLLYEYFTVYNELTKVKYVTEGMRKPAFLSGEQKKAIVDLLFKTN | |
| | RKVTVKQLKEDYFKKIECFDSVEISGVEDRFNASLGTYHDLLKIIKDKDFLDNEENEDILEDIVLTLT | |
| | LFEDREMIEERLKTYAHLFDDKVMKQLKRRRYTGWGRLSRKLINGIRDKQSGKTILDFLKSDGFANRN | |
| | FMQLIHDDSLTFKEDIQKAQVSGQGDSLHEHIANLAGSPAIKKGILQTVKVVDELVKVMGRHKPENIV | |
| | IEMARENQTTQKGQKNSRERMKRIEEGIKELGSQILKEHPVENTQLQNEKLYLYYLQNGRDMYVDQEL | |
| | DINRLSDYDVDHIVPQSFLKDDSIDNKVLTRSDKNRGKSDNVPSEEVVKKMKNYWRQLLNAKLITQRK | |
| | FDNLTKAERGGLSELDKAGFIKRQLVETRQITKHVAQILDSRMNTKYDENDKLIREVKVITLKSKLVS | |
| | DFRKDFQFYKVREINNYHHAHDAYLNAVVGTALIKKYPKLESEFVYGDYKVYDVRKMIAKSEQEIGKA | |
| | TAKYFFYSNIMNFFKTEITLANGEIRKRPLIETNGETGEIVWDKGRDFATVRKVLSMPQVNIVKKTEV | |
| | QTGGFSKESILPKRNSDKLIARKKDWDPKKYGGFDSPTVAYSVLVVAKVEKGKSKKLKSVKELLGITI | |
| | MERSSFEKNPIDFLEAKGYKEVKKDLIIKLPKYSLFELENGRKRMLASAGELQKGNELALPSKYVNFL | |
| | YLASHYEKLKGSPEDNEQKQLFVEQHKHYLDEIIEQISEFSKRVILADANLDKVLSAYNKHRDKPIRE | |
| | QAENIIHLFTLTNLGAPAAFKYFDTTIDRKRYTSTKEVLDATLIHQSITGLYETRIDLSQLGGDSGGS | |
| | PKKKRKV | |
| pNMG-621 | MSEVEFSHEYWMRHALTLAKRAWDEREVPVGAVLVHNNRVIGEGWNRPIGRHDPTAHAEIMALRQGGL | 193 |
| | VMQNYRLIDATLYVTLEPCVMCAGAMIHSRIGRVVFGARDAKTGAAGSLMDVLHHPGMNHRVEITEGI | |
| | LADECAALLSDFFRMRRQEIKAQKKAQSSTDSGGSSGGSSGSETPGTSESATPESSGGSSGGSSEVEF | |
| | SHEYWMRHALTLAKRAWDEREVPVGAVLVLNNRVIGEGWNRAIGLHDPTAHAEIMALRQGGLVMQNYR | |
| | LIDATLYVTFEPCVMCAGAMIHSRIGRVVFGVRNAKTGAAGSLMDVLHYPGMNHRVEITEGILADECA | |
| | ALLCYFFRMPRQVFNAQKKAQSSTDSGGSSGGSSGSETPGTSESATPESDKKYSIGLAIGTNSVGWAV | |
| | ITDEYKVPSKKFKVLGNTDRHSIKKNLIGALLFDSGETAEATRLKRTARRRYTRRKNRICYLQEIFSN | |
| | EMAKVDDSFFHRLEESFLVEEDKKHERHPIFGNIVDEVAYHEKYPTIYHLRKKLVDSTDKADLRLIYL | |
| | ALAHMIKFRGHFLIEGDLNPDNSDVDKLFIQLVQTYNQLFEENPINASGVDAKAILSARLSKSRRLEN | |
| | LIAQLPGEKKNGLFGNLIALSLGLTPNFKSNFDLAEDAKLQLSKDTYDDDLDNLLAQIGDQYADLFLA | |
| | AKNLSDAILLSDILRVNTEITKAPLSASMIKRYDEHHQDLTLLKALVRQQLPEKYKEIFFDQSKNGYA | |
| | GYIDGGASQEEFYKFIKPILEKMDGTEELLVKLNREDLLRKQRTFDNGSIPHQIHLGELHAILRRQED | |
| | FYPFLKDNREKIEKILTFRIPYYVGPLARGNSRFAWMTRKSEETITPWNFEEVVDKGASAQSFIERMT | |
| | NFDKNLPNEKVLPKHSLLYEYFTVYNELTKVKYVTEGMRKPAFLSGEQKKAIVDLLFKTNRKVTVKQL | |
| | KEDYFKKIECFDSVEISGVEDRFNASLGTYHDLLKIIKDKDFLDNEENEDILEDIVLTLTLFEDREMI | |
| | EERLKTYAHLFDDKVMKQLKRRRYTGWGRLSRKLINGIRDKQSGKTILDFLKSDGFANRNFMQLIHDD | |
| | SLTFKEDIQKAQVSGQGDSLHEHIANLAGSPAIKKGILQTVKVVDELVKVMGRHKPENIVIEMARENQ | |
| | TTQKGQKNSRERMKRIEEGIKELGSQILKEHPVENTQLQNEKLYLYYLQNGRDMYVDQELDINRLSDY | |
| | DVDHIVPQSFLKDDSIDNKVLTRSDKNRGKSDNVPSEEVVKKMKNYWRQLLNAKLITQRKFDNLTKAE | |
| | RGGLSELDKAGFIKRQLVETRQITKHVAQILDSRMNTKYDENDKLIREVKVITLKSKLVSDFRKDFQF | |
| | YKVREINNYHHAHDAYLNAVVGTALIKKYPKLESEFVYGDYKVYDVRKMIAKSEQEIGKATAKYFFYS | |
| | NIMNFFKTEITLANGEIRKRPLIETNGETGEIVWDKGRDFATVRKVLSMPQVNIVKKTEVQTGGFSKE | |
| | SILPKRNSDKLIARKKDWDPKKYGGFDSPTVAYSVLVVAKVEKGKSKKLKSVKELLGITIMERSSFEK | |
| | NPIDFLEAKGYKEVKKDLIIKLPKYSLFELENGRKRMLASAGELQKGNELALPSKYVNFLYLASHYEK | |
| | LKGSPEDNEQKQLFVEQHKHYLDEIIEQISEFSKRVILADANLDKVLSAYNKHRDKPIREQAENIIHL | |
| | FTLTNLGAPAAFKYFDTTIDRKRYTSTKEVLDATLIHQSITGLYETRIDLSQLGGDSGGSPKKKRKV | |
| pNMG-622 | MSEVEFSHEYWMRHALTLAKRAWDEREVPVGAVLVHNNRVIGEGWNRPIGRHDPTAHAEIMALRQGGL | 194 |
| | VMQNYRLIDATLYVTLEPCVMCAGAMIHSRIGRVVFGARDAKTGAAGSLMDVLHHPGMNHRVEITEGI | |
| | LADECAALLSDFFRMRRQEIKAQKKAQSSTDSGGSSGGSSGSETPGTSESATPESSGGSSGGSSEVEF | |
| | SHEYWMRHALTLAKRAWDEREVPVGAVLVLNNRVIGEGWNRAIGLHDPTAHAEIMALRQGGLVMQNYR | |
| | LIDATLYVTFEPCVMCAGAMIHSRIGRVVFGVRNAKTGAAGSLMDVLHYPGMNHRVEITEGILADECN | |
| | ALLCYFFRMPRQVFNAQKKAQSSTDSGGSSGGSSGSETPGTSESATPESDKKYSIGLAIGTNSVGWAV | |
| | ITDEYKVPSKKFKVLGNTDRHSIKKNLIGALLFDSGETAEATRLKRTARRRYTRRKNRICYLQEIFSN | |
| | EMAKVDDSFFHRLEESFLVEEDKKHERHPIFGNIVDEVAYHEKYPTIYHLRKKLVDSTDKADLRLIYL | |
| | ALAHMIKFRGHFLIEGDLNPDNSDVDKLFIQLVQTYNQLFEENPINASGVDAKAILSARLSKSRRLEN | |
| | LIAQLPGEKKNGLFGNLIALSLGLTPNFKSNFDLAEDAKLQLSKDTYDDDLDNLLAQIGDQYADLFLA | |
| | AKNLSDAILLSDILRVNTEITKAPLSASMIKRYDEHHQDLTLLKALVRQQLPEKYKEIFFDQSKNGYA | |
| | GYIDGGASQEEFYKFIKPILEKMDGTEELLVKLNREDLLRKQRTFDNGSIPHQIHLGELHAILRRQED | |
| | FYPFLKDNREKIEKILTFRIPYYVGPLARGNSRFAWMTRKSEETITPWNFEEVVDKGASAQSFIERMT | |
| | NFDKNLPNEKVLPKHSLLYEYFTVYNELTKVKYVTEGMRKPAFLSGEQKKAIVDLLFKTNRKVTVKQL | |
| | KEDYFKKIECFDSVEISGVEDRFNASLGTYHDLLKIIKDKDFLDNEENEDILEDIVLTLTLFEDREMI | |
| | EERLKTYAHLFDDKVMKQLKRRRYTGWGRLSRKLINGIRDKQSGKTILDFLKSDGFANRNFMQLIHDD | |
| | SLTFKEDIQKAQVSGQGDSLHEHIANLAGSPAIKKGILQTVKVVDELVKVMGRHKPENIVIEMARENQ | |
| | TTQKGQKNSRERMKRIEEGIKELGSQILKEHPVENTQLQNEKLYLYYLQNGRDMYVDQELDINRLSDY | |
| | DVDHIVPQSFLKDDSIDNKVLTRSDKNRGKSDNVPSEEVVKKMKNYWRQLLNAKLITQRKFDNLTKAE | |
| | RGGLSELDKAGFIKRQLVETRQITKHVAQILDSRMNTKYDENDKLIREVKVITLKSKLVSDFRKDFQF | |
| | YKVREINNYHHAHDAYLNAVVGTALIKKYPKLESEFVYGDYKVYDVRKMIAKSEQEIGKATAKYFFYS | |
| | NIMNFFKTEITLANGEIRKRPLIETNGETGEIVWDKGRDFATVRKVLSMPQVNIVKKTEVQTGGFSKE | |
| | SILPKRNSDKLIARKKDWDPKKYGGFDSPTVAYSVLVVAKVEKGKSKKLKSVKELLGITIMERSSFEK | |
| | NPIDFLEAKGYKEVKKDLIIKLPKYSLFELENGRKRMLASAGELQKGNELALPSKYVNFLYLASHYEK | |
| | LKGSPEDNEQKQLFVEQHKHYLDEIIEQISEFSKRVILADANLDKVLSAYNKHRDKPIREQAENIIHL | |
| | FTLTNLGAPAAFKYFDTTIDRKRYTSTKEVLDATLIHQSITGLYETRIDLSQLGGDSGGSPKKKRKV | |
| pNMG-623 | MSEVEFSHEYWMRHALTLAKRAWDEREVPVGAVLVHNNRVIGEGWNRPIGRHDPTAHAEIMALRQGGL | 195 |
| | VMQNYRLIDATLYVTLEPCVMCAGAMIHSRIGRVVFGARDAKTGAAGSLMDVLHHPGMNHRVEITEGI | |
| | LADECAALLSDFFRMRRQEIKAQKKAQSSTDSGGSSGGSSGSETPGTSESATPESSGGSSGGSSEVEF | |
| | SHEYWMRHALTLAKRALDEREVPVGAVLVLNNRVIGEGWNRAIGLHDPTAHAEIMALRQGGLVMQNYR | |
| | LIDATLYVTFEPCVMCAGAMIHSRIGRVVFGVRNAKTGAAGSLMDVLHYPGMNHRVEITEGILADECA | |
| | ALLCYFFRMPRQVFNAQKKAQSSTDSGGSSGGSSGSETPGTSESATPESDKKYSIGLAIGTNSVGWAV | |
| | ITDEYKVPSKKFKVLGNTDRHSIKKNLIGALLFDSGETAEATRLKRTARRRYTRRKNRICYLQEIFSN | |
| | EMAKVDDSFFHRLEESFLVEEDKKHERHPIFGNIVDEVAYHEKYPTIYHLRKKLVDSTDKADLRLIYL | |
| | ALAHMIKFRGHFLIEGDLNPDNSDVDKLFIQLVQTYNQLFEENPINASGVDAKAILSARLSKSRRLEN | |
| | LIAQLPGEKKNGLFGNLIALSLGLTPNFKSNFDLAEDAKLQLSKDTYDDDLDNLLAQIGDQYADLFLA | |
| | AKNLSDAILLSDILRVNTEITKAPLSASMIKRYDEHHQDLTLLKALVRQQLPEKYKEIFFDQSKNGYA | |
| | GYIDGGASQEEFYKFIKPILEKMDGTEELLVKLNREDLLRKQRTFDNGSIPHQIHLGELHAILRRQED | |
| | FYPFLKDNREKIEKILTFRIPYYVGPLARGNSRFAWMTRKSEETITPWNFEEVVDKGASAQSFIERMT | |
| | NFDKNLPNEKVLPKHSLLYEYFTVYNELTKVKYVTEGMRKPAFLSGEQKKAIVDLLFKTNRKVTVKQL | |
| | KEDYFKKIECFDSVEISGVEDRFNASLGTYHDLLKIIKDKDFLDNEENEDILEDIVLTLTLFEDREMI | |
| | EERLKTYAHLFDDKVMKQLKRRRYTGWGRLSRKLINGIRDKQSGKTILDFLKSDGFANRNFMQLIHDD | |
| | SLTFKEDIQKAQVSGQGDSLHEHIANLAGSPAIKKGILQTVKVVDELVKVMGRHKPENIVIEMARENQ | |
| | TTQKGQKNSRERMKRIEEGIKELGSQILKEHPVENTQLQNEKLYLYYLQNGRDMYVDQELDINRLSDY | |
| | DVDHIVPQSFLKDDSIDNKVLTRSDKNRGKSDNVPSEEVVKKMKNYWRQLLNAKLITQRKFDNLTKAE | |
| | RGGLSELDKAGFIKRQLVETRQITKHVAQILDSRMNTKYDENDKLIREVKVITLKSKLVSDFRKDFQF | |
| | YKVREINNYHHAHDAYLNAVVGTALIKKYPKLESEFVYGDYKVYDVRKMIAKSEQEIGKATAKYFFYS | |
| | NIMNFFKTEITLANGEIRKRPLIETNGETGEIVWDKGRDFATVRKVLSMPQVNIVKKTEVQTGGFSKE | |
| | SILPKRNSDKLIARKKDWDPKKYGGFDSPTVAYSVLVVAKVEKGKSKKLKSVKELLGITIMERSSFEK | |
| | NPIDFLEAKGYKEVKKDLIIKLPKYSLFELENGRKRMLASAGELQKGNELALPSKYVNFLYLASHYEK | |
| | LKGSPEDNEQKQLFVEQHKHYLDEIIEQISEFSKRVILADANLDKVLSAYNKHRDKPIREQAENIIHL | |
| | FTLTNLGAPAAFKYFDTTIDRKRYTSTKEVLDATLIHQSITGLYETRIDLSQLGGDSGGSPKKKRKV | |
| ABE6.3 | MSEVEFSHEYWMRHALTLAKRAWDEREVPVGAVLVHNNRVIGEGWNRPIGRHDPTAHAEIMALRQGGL | 196 |
| | VMQNYRLIDATLYVTLEPCVMCAGAMIHSRIGRVVFGARDAKTGAAGSLMDVLHHPGMNHRVEITEGI | |
| | LADECAALLSDFFRMRRQEIKAQKKAQSSTDSGGSSGGSSGSETPGTSESATPESSGGSSGGSSEVEF | |
| | SHEYWMRHALTLAKRAWDEREVPVGAVLVLNNRVIGEGWNRSIGLHDPTAHAEIMALRQGGLVMQNYR | |
| | LIDATLYVTFEPCVMCAGAMIHSRIGRVVFGVRNAKTGAAGSLMDVLHYPGMNHRVEITEGILADECA | |
| | ALLCYFFRMRRQVFNAQKKAQSSTDSGGSSGGSSGSETPGTSESATPESSGGSSGGSDKKYSIGLAIG | |
| | TNSVGWAVITDEYKVPSKKFKVLGNTDRHSIKKNLIGALLFDSGETAEATRLKRTARRRYTRRKNRIC | |
| | YLQEIFSNEMAKVDDSFFHRLEESFLVEEDKKHERHPIFGNIVDEVAYHEKYPTIYHLRKKLVDSTDK | |
| | ADLRLIYLALAHMIKFRGHFLIEGDLNPDNSDVDKLFIQLVQTYNQLFEENPINASGVDAKAILSARL | |
| | SKSRRLENLIAQLPGEKKNGLFGNLIALSLGLTPNFKSNFDLAEDAKLQLSKDTYDDDLDNLLAQIGD | |
| | QYADLFLAAKNLSDAILLSDILRVNTEITKAPLSASMIKRYDEHHQDLTLLKALVRQQLPEKYKEIFF | |
| | DQSKNGYAGYIDGGASQEEFYKFIKPILEKMDGTEELLVKLNREDLLRKQRTFDNGSIPHQIHLGELH | |
| | AILRRQEDFYPFLKDNREKIEKILTFRIPYYVGPLARGNSRFAWMTRKSEETITPWNFEEVVDKGASA | |
| | QSFIERMTNFDKNLPNEKVLPKHSLLYEYFTVYNELTKVKYVTEGMRKPAFLSGEQKKAIVDLLFKTN | |
| | RKVTVKQLKEDYFKKIECFDSVEISGVEDRFNASLGTYHDLLKIIKDKDFLDNEENEDILEDIVLTLT | |
| | LFEDREMIEERLKTYAHLFDDKVMKQLKRRRYTGWGRLSRKLINGIRDKQSGKTILDFLKSDGFANRN | |
| | FMQLIHDDSLTFKEDIQKAQVSGQGDSLHEHIANLAGSPAIKKGILQTVKVVDELVKVMGRHKPENIV | |
| | IEMARENQTTQKGQKNSRERMKRIEEGIKELGSQILKEHPVENTQLQNEKLYLYYLQNGRDMYVDQEL | |
| | DINRLSDYDVDHIVPQSFLKDDSIDNKVLTRSDKNRGKSDNVPSEEVVKKMKNYWRQLLNAKLITQRK | |
| | FDNLTKAERGGLSELDKAGFIKRQLVETRQITKHVAQILDSRMNTKYDENDKLIREVKVITLKSKLVS | |
| | DFRKDFQFYKVREINNYHHAHDAYLNAVVGTALIKKYPKLESEFVYGDYKVYDVRKMIAKSEQEIGKA | |
| | TAKYFFYSNIMNFFKTEITLANGEIRKRPLIETNGETGEIVWDKGRDFATVRKVLSMPQVNIVKKTEV | |
| | QTGGFSKESILPKRNSDKLIARKKDWDPKKYGGFDSPTVAYSVLVVAKVEKGKSKKLKSVKELLGITI | |
| | MERSSFEKNPIDFLEAKGYKEVKKDLIIKLPKYSLFELENGRKRMLASAGELQKGNELALPSKYVNFL | |
| | YLASHYEKLKGSPEDNEQKQLFVEQHKHYLDEIIEQISEFSKRVILADANLDKVLSAYNKHRDKPIRE | |
| | QAENIIHLFTLTNLGAPAAFKYFDTTIDRKRYTSTKEVLDATLIHQSITGLYETRIDLSQLGGDSGGS | |
| | PKKKRKV | |
| ABE6.4 | MSEVEFSHEYWMRHALTLAKRAWDEREVPVGAVLVHNNRVIGEGWNRPIGRHDPTAHAEIMALRQGGL | 197 |
| | VMQNYRLIDATLYVTLEPCVMCAGAMIHSRIGRVVFGARDAKTGAAGSLMDVLHHPGMNHRVEITEGI | |
| | LADECAALLSDFFRMRRQEIKAQKKAQSSTDSGGSSGGSSGSETPGTSESATPESSGGSSGGSSEVEF | |
| | SHEYWMRHALTLAKRAWDEREVPVGAVLVLNNRVIGEGWNRSIGLHDPTAHAEIMALRQGGLVMQNYR | |
| | LIDATLYVTFEPCVMCAGAMIHSRIGRVVFGVRNAKTGAAGSLMDVLHYPGMNHRVEITEGILADECN | |
| | ALLCYFFRMRRQVFNAQKKAQSSTDSGGSSGGSSGSETPGTSESATPESSGGSSGGSDKKYSIGLAIG | |
| | TNSVGWAVITDEYKVPSKKFKVLGNTDRHSIKKNLIGALLFDSGETAEATRLKRTARRRYTRRKNRIC | |
| | YLQEIFSNEMAKVDDSFFHRLEESFLVEEDKKHERHPIFGNIVDEVAYHEKYPTIYHLRKKLVDSTDK | |
| | ADLRLIYLALAHMIKFRGHFLIEGDLNPDNSDVDKLFIQLVQTYNQLFEENPINASGVDAKAILSARL | |
| | SKSRRLENLIAQLPGEKKNGLFGNLIALSLGLTPNFKSNFDLAEDAKLQLSKDTYDDDLDNLLAQIGD | |
| | QYADLFLAAKNLSDAILLSDILRVNTEITKAPLSASMIKRYDEHHQDLTLLKALVRQQLPEKYKEIFF | |
| | DQSKNGYAGYIDGGASQEEFYKFIKPILEKMDGTEELLVKLNREDLLRKQRTFDNGSIPHQIHLGELH | |
| | AILRRQEDFYPFLKDNREKIEKILTFRIPYYVGPLARGNSRFAWMTRKSEETITPWNFEEVVDKGASA | |
| | QSFIERMTNFDKNLPNEKVLPKHSLLYEYFTVYNELTKVKYVTEGMRKPAFLSGEQKKAIVDLLFKTN | |
| | RKVTVKQLKEDYFKKIECFDSVEISGVEDRFNASLGTYHDLLKIIKDKDFLDNEENEDILEDIVLTLT | |
| | LFEDREMIEERLKTYAHLFDDKVMKQLKRRRYTGWGRLSRKLINGIRDKQSGKTILDFLKSDGFANRN | |
| | FMQLIHDDSLTFKEDIQKAQVSGQGDSLHEHIANLAGSPAIKKGILQTVKVVDELVKVMGRHKPENIV | |
| | IEMARENQTTQKGQKNSRERMKRIEEGIKELGSQILKEHPVENTQLQNEKLYLYYLQNGRDMYVDQEL | |
| | DINRLSDYDVDHIVPQSFLKDDSIDNKVLTRSDKNRGKSDNVPSEEVVKKMKNYWRQLLNAKLITQRK | |
| | FDNLTKAERGGLSELDKAGFIKRQLVETRQITKHVAQILDSRMNTKYDENDKLIREVKVITLKSKLVS | |
| | DFRKDFQFYKVREINNYHHAHDAYLNAVVGTALIKKYPKLESEFVYGDYKVYDVRKMIAKSEQEIGKA | |
| | TAKYFFYSNIMNFFKTEITLANGEIRKRPLIETNGETGEIVWDKGRDFATVRKVLSMPQVNIVKKTEV | |
| | QTGGFSKESILPKRNSDKLIARKKDWDPKKYGGFDSPTVAYSVLVVAKVEKGKSKKLKSVKELLGITI | |
| | MERSSFEKNPIDFLEAKGYKEVKKDLIIKLPKYSLFELENGRKRMLASAGELQKGNELALPSKYVNFL | |
| | YLASHYEKLKGSPEDNEQKQLFVEQHKHYLDEIIEQISEFSKRVILADANLDKVLSAYNKHRDKPIRE | |
| | QAENIIHLFTLTNLGAPAAFKYFDTTIDRKRYTSTKEVLDATLIHQSITGLYETRIDLSQLGGDSGGS | |
| | PKKKRKV | |
| ABE7.8 | MSEVEFSHEYWMRHALTLAKRAWDEREVPVGAVLVHNNRVIGEGWNRPIGRHDPTAHAEIMALRQGGL | 198 |
| | VMQNYRLIDATLYVTLEPCVMCAGAMIHSRIGRVVFGARDAKTGAAGSLMDVLHHPGMNHRVEITEGI | |
| | LADECAALLSDFFRMRRQEIKAQKKAQSSTDSGGSSGGSSGSETPGTSESATPESSGGSSGGSSEVEF | |
| | SHEYWMRHALTLAKRALDEREVPVGAVLVLNNRVIGEGWNRAIGLHDPTAHAEIMALRQGGLVMQNYR | |
| | LIDATLYVTFEPCVMCAGAMIHSRIGRVVFGVRNAKTGAAGSLMDVLHYPGMNHRVEITEGILADECN | |
| | ALLCYFFRMRRQVFNAQKKAQSSTDSGGSSGGSSGSETPGTSESATPESSGGSSGGSDKKYSIGLAIG | |
| | TNSVGWAVITDEYKVPSKKFKVLGNTDRHSIKKNLIGALLFDSGETAEATRLKRTARRRYTRRKNRIC | |
| | YLQEIFSNEMAKVDDSFFHRLEESFLVEEDKKHERHPIFGNIVDEVAYHEKYPTIYHLRKKLVDSTDK | |
| | ADLRLIYLALAHMIKFRGHFLIEGDLNPDNSDVDKLFIQLVQTYNQLFEENPINASGVDAKAILSARL | |
| | SKSRRLENLIAQLPGEKKNGLFGNLIALSLGLTPNFKSNFDLAEDAKLQLSKDTYDDDLDNLLAQIGD | |
| | QYADLFLAAKNLSDAILLSDILRVNTEITKAPLSASMIKRYDEHHQDLTLLKALVRQQLPEKYKEIFF | |
| | DQSKNGYAGYIDGGASQEEFYKFIKPILEKMDGTEELLVKLNREDLLRKQRTFDNGSIPHQIHLGELH | |
| | AILRRQEDFYPFLKDNREKIEKILTFRIPYYVGPLARGNSRFAWMTRKSEETITPWNFEEVVDKGASA | |
| | QSFIERMTNFDKNLPNEKVLPKHSLLYEYFTVYNELTKVKYVTEGMRKPAFLSGEQKKAIVDLLFKTN | |
| | RKVTVKQLKEDYFKKIECFDSVEISGVEDRFNASLGTYHDLLKIIKDKDFLDNEENEDILEDIVLTLT | |
| | LFEDREMIEERLKTYAHLFDDKVMKQLKRRRYTGWGRLSRKLINGIRDKQSGKTILDFLKSDGFANRN | |
| | FMQLIHDDSLTFKEDIQKAQVSGQGDSLHEHIANLAGSPAIKKGILQTVKVVDELVKVMGRHKPENIV | |
| | IEMARENQTTQKGQKNSRERMKRIEEGIKELGSQILKEHPVENTQLQNEKLYLYYLQNGRDMYVDQEL | |
| | DINRLSDYDVDHIVPQSFLKDDSIDNKVLTRSDKNRGKSDNVPSEEVVKKMKNYWRQLLNAKLITQRK | |
| | FDNLTKAERGGLSELDKAGFIKRQLVETRQITKHVAQILDSRMNTKYDENDKLIREVKVITLKSKLVS | |
| | DFRKDFQFYKVREINNYHHAHDAYLNAVVGTALIKKYPKLESEFVYGDYKVYDVRKMIAKSEQEIGKA | |
| | TAKYFFYSNIMNFFKTEITLANGEIRKRPLIETNGETGEIVWDKGRDFATVRKVLSMPQVNIVKKTEV | |
| | QTGGFSKESILPKRNSDKLIARKKDWDPKKYGGFDSPTVAYSVLVVAKVEKGKSKKLKSVKELLGITI | |
| | MERSSFEKNPIDFLEAKGYKEVKKDLIIKLPKYSLFELENGRKRMLASAGELQKGNELALPSKYVNFL | |
| | YLASHYEKLKGSPEDNEQKQLFVEQHKHYLDEIIEQISEFSKRVILADANLDKVLSAYNKHRDKPIRE | |
| | QAENIIHLFTLTNLGAPAAFKYFDTTIDRKRYTSTKEVLDATLIHQSITGLYETRIDLSQLGGDSGGS | |
| | PKKKRKVc | |
| ABE7.9 | MSEVEFSHEYWMRHALTLAKRAWDEREVPVGAVLVHNNRVIGEGWNRPIGRHDPTAHAEIMALRQGGL | 199 |
| | VMQNYRLIDATLYVTLEPCVMCAGAMIHSRIGRVVFGARDAKTGAAGSLMDVLHHPGMNHRVEITEGI | |
| | LADECAALLSDFFRMRRQEIKAQKKAQSSTDSGGSSGGSSGSETPGTSESATPESSGGSSGGSSEVEF | |
| | SHEYWMRHALTLAKRALDEREVPVGAVLVLNNRGEGWNRAIGLHDPTAHAEIMALRQGGLVMQNYRLI | |
| | DATLYVTFEPCVMCAGAMIHSRIGRVVFGVRNAKTGAAGSLMDVLHYPGMNHRVEITEGILADECNAL | |
| | LCYFFRMPRQVFNAQKKAQSSTDSGGSSGGSSGSETPGTSESATPESSGGSSGGSDKKYSIGLAIGTN | |
| | SVGWAVITDEYKVPSKKFKVLGNTDRHSIKKNLIGALLFDSGETAEATRLKRTARRRYTRRKNRICYL | |
| | QEIFSNEMAKVDDSFFHRLEESFLVEEDKKHERHPIFGNIVDEVAYHEKYPTIYHLRKKLVDSTDKAD | |
| | LRLIYLALAHMIKFRGHFLIEGDLNPDNSDVDKLFIQLVQTYNQLFEENPINASGVDAKAILSARLSK | |
| | SRRLENLIAQLPGEKKNGLFGNLIALSLGLTPNFKSNFDLAEDAKLQLSKDTYDDDLDNLLAQIGDQY | |
| | ADLFLAAKNLSDAILLSDILRVNTEITKAPLSASMIKRYDEHHQDLTLLKALVRQQLPEKYKEIFFDQ | |
| | SKNGYAGYIDGGASQEEFYKFIKPILEKMDGTEELLVKLNREDLLRKQRTFDNGSIPHQIHLGELHAI | |
| | LRRQEDFYPFLKDNREKIEKILTFRIPYYVGPLARGNSRFAWMTRKSEETITPWNFEEVVDKGASAQS | |
| | FIERMTNFDKNLPNEKVLPKHSLLYEYFTVYNELTKVKYVTEGMRKPAFLSGEQKKAIVDLLFKTNRK | |
| | VTVKQLKEDYFKKIECFDSVEISGVEDRFNASLGTYHDLLKIIKDKDFLDNEENEDILEDIVLTLTLF | |
| | EDREMIEERLKTYAHLFDDKVMKQLKRRRYTGWGRLSRKLINGIRDKQSGKTILDFLKSDGFANRNFM | |
| | QLIHDDSLTFKEDIQKAQVSGQGDSLHEHIANLAGSPAIKKGILQTVKVVDELVKVMGRHKPENIVIE | |
| | MARENQTTQKGQKNSRERMKRIEEGIKELGSQILKEHPVENTQLQNEKLYLYYLQNGRDMYVDQELDI | |
| | NRLSDYDVDHIVPQSFLKDDSIDNKVLTRSDKNRGKSDNVPSEEVVKKMKNYWRQLLNAKLITQRKFD | |
| | NLTKAERGGLSELDKAGFIKRQLVETRQITKHVAQILDSRMNTKYDENDKLIREVKVITLKSKLVSDF | |
| | RKDFQFYKVREINNYHHAHDAYLNAVVGTALIKKYPKLESEFVYGDYKVYDVRKMIAKSEQEIGKATA | |
| | KYFFYSNIMNFFKTEITLANGEIRKRPLIETNGETGEIVWDKGRDFATVRKVLSMPQVNIVKKTEVQT | |
| | GGFSKESILPKRNSDKLIARKKDWDPKKYGGFDSPTVAYSVLVVAKVEKGKSKKLKSVKELLGITIME | |
| | RSSFEKNPIDFLEAKGYKEVKKDLIIKLPKYSLFELENGRKRMLASAGELQKGNELALPSKYVNFLYL | |
| | ASHYEKLKGSPEDNEQKQLFVEQHKHYLDEIIEQISEFSKRVILADANLDKVLSAYNKHRDKPIREQA | |
| | ENIIHLFTLTNLGAPAAFKYFDTTIDRKRYTSTKEVLDATLIHQSITGLYETRIDLSQLGGDSGGSPK | |
| | KKRKV | |
| ABE7.10 | MSEVEFSHEYWMRHALTLAKRAWDEREVPVGAVLVHNNRVIGEGWNRPIGRHDPTAHAEIMALRQGGL | 200 |
| | VMQNYRLIDATLYVTLEPCVMCAGAMIHSRIGRVVFGARDAKTGAAGSLMDVLHHPGMNHRVEITEGI | |
| | LADECAALLSDFFRMRRQEIKAQKKAQSSTDSGGSSGGSSGSETPGTSESATPESSGGSSGGSSEVEF | |
| | SHEYWMRHALTLAKRARDEREVPVGAVLVLNNRVIGEGWNRAIGLHDPTAHAEIMALRQGGLVMQNYR | |
| | LIDATLYVTFEPCVMCAGAMIHSRIGRVVFGVRNAKTGAAGSLMDVLHYPGMNHRVEITEGILADECA | |
| | ALLCYFFRMPRQVFNAQKKAQSSTDSGGSSGGSSGSETPGTSESATPESSGGSSGGSDKKYSIGLAIG | |
| | TNSVGWAVITDEYKVPSKKFKVLGNTDRHSIKKNLIGALLFDSGETAEATRLKRTARRRYTRRKNRIC | |
| | YLQEIFSNEMAKVDDSFFHRLEESFLVEEDKKHERHPIFGNIVDEVAYHEKYPTIYHLRKKLVDSTDK | |
| | ADLRLIYLALAHMIKFRGHFLIEGDLNPDNSDVDKLFIQLVQTYNQLFEENPINASGVDAKAILSARL | |
| | SKSRRLENLIAQLPGEKKNGLFGNLIALSLGLTPNFKSNFDLAEDAKLQLSKDTYDDDLDNLLAQIGD | |
| | QYADLFLAAKNLSDAILLSDILRVNTEITKAPLSASMIKRYDEHHQDLTLLKALVRQQLPEKYKEIFF | |
| | DQSKNGYAGYIDGGASQEEFYKFIKPILEKMDGTEELLVKLNREDLLRKQRTFDNGSIPHQIHLGELH | |
| | AILRRQEDFYPFLKDNREKIEKILTFRIPYYVGPLARGNSRFAWMTRKSEETITPWNFEEVVDKGASA | |
| | QSFIERMTNFDKNLPNEKVLPKHSLLYEYFTVYNELTKVKYVTEGMRKPAFLSGEQKKAIVDLLFKTN | |
| | RKVTVKQLKEDYFKKIECFDSVEISGVEDRFNASLGTYHDLLKIIKDKDFLDNEENEDILEDIVLTLT | |
| | LFEDREMIEERLKTYAHLFDDKVMKQLKRRRYTGWGRLSRKLINGIRDKQSGKTILDFLKSDGFANRN | |
| | FMQLIHDDSLTFKEDIQKAQVSGQGDSLHEHIANLAGSPAIKKGILQTVKVVDELVKVMGRHKPENIV | |
| | IEMARENQTTQKGQKNSRERMKRIEEGIKELGSQILKEHPVENTQLQNEKLYLYYLQNGRDMYVDQEL | |
| | DINRLSDYDVDHIVPQSFLKDDSIDNKVLTRSDKNRGKSDNVPSEEVVKKMKNYWRQLLNAKLITQRK | |
| | FDNLTKAERGGLSELDKAGFIKRQLVETRQITKHVAQILDSRMNTKYDENDKLIREVKVITLKSKLVS | |
| | DFRKDFQFYKVREINNYHHAHDAYLNAVVGTALIKKYPKLESEFVYGDYKVYDVRKMIAKSEQEIGKA | |
| | TAKYFFYSNIMNFFKTEITLANGEIRKRPLIETNGETGEIVWDKGRDFATVRKVLSMPQVNIVKKTEV | |
| | QTGGFSKESILPKRNSDKLIARKKDWDPKKYGGFDSPTVAYSVLVVAKVEKGKSKKLKSVKELLGITI | |
| | MERSSFEKNPIDFLEAKGYKEVKKDLIIKLPKYSLFELENGRKRMLASAGELQKGNELALPSKYVNFL | |
| | YLASHYEKLKGSPEDNEQKQLFVEQHKHYLDEIIEQISEFSKRVILADANLDKVLSAYNKHRDKPIRE | |
| | QAENIIHLFTLTNLGAPAAFKYFDTTIDRKRYTSTKEVLDATLIHQSITGLYETRIDLSQLGGDSGGS | |
| | PKKKRKV | |
| ABEmax (7.10) | MKRTADGSEFESPKKKRKVSEVEFSHEYWMRHALTLAKRAWDEREVPVGAVLVHNNRVIGEGWNRPIG | 201 |
| | RHDPTAHAEIMALRQGGLVMQNYRLIDATLYVTLEPCVMCAGAMIHSRIGRVVFGARDAKTGAAGSLM | |
| | DVLHHPGMNHRVEITEGILADECAALLSDFFRMRRQEIKAQKKAQSSTDSGGSSGGSSGSETPGTSES | |
| | ATPESSGGSSGGSSEVEFSHEYWMRHALTLAKRARDEREVPVGAVLVLNNRVIGEGWNRAIGLHDPTA | |
| | HAEIMALRQGGLVMQNYRLIDATLYVTFEPCVMCAGAMIHSRIGRVVFGVRNAKTGAAGSLMDVLHYP | |
| | GMNHRVEITEGILADECAALLCYFFRMPRQVFNAQKKAQSSTDSGGSSGGSSGSETPGTSESATPESS | |
| | GGSSGGSDKKYSIGLAIGTNSVGWAVITDEYKVPSKKFKVLGNTDRHSIKKNLIGALLFDSGETAEAT | |
| | RLKRTARRRYTRRKNRICYLQEIFSNEMAKVDDSFFHRLEESFLVEEDKKHERHPIFGNIVDEVAYHE | |
| | KYPTIYHLRKKLVDSTDKADLRLIYLALAHMIKFRGHFLIEGDLNPDNSDVDKLFIQLVQTYNQLFEE | |
| | NPINASGVDAKAILSARLSKSRRLENLIAQLPGEKKNGLFGNLIALSLGLTPNFKSNFDLAEDAKLQL | |
| | SKDTYDDDLDNLLAQIGDQYADLFLAAKNLSDAILLSDILRVNTEITKAPLSASMIKRYDEHHQDLTL | |
| | LKALVRQQLPEKYKEIFFDQSKNGYAGYIDGGASQEEFYKFIKPILEKMDGTEELLVKLNREDLLRKQ | |
| | RTFDNGSIPHQIHLGELHAILRRQEDFYPFLKDNREKIEKILTFRIPYYVGPLARGNSRFAWMTRKSE | |
| | ETITPWNFEEVVDKGASAQSFIERMTNFDKNLPNEKVLPKHSLLYEYFTVYNELTKVKYVTEGMRKPA | |
| | FLSGEQKKAIVDLLFKTNRKVTVKQLKEDYFKKIECFDSVEISGVEDRFNASLGTYHDLLKIIKDKDF | |
| | LDNEENEDILEDIVLTLTLFEDREMIEERLKTYAHLFDDKVMKQLKRRRYTGWGRLSRKLINGIRDKQ | |
| | SGKTILDFLKSDGFANRNFMQLIHDDSLTFKEDIQKAQVSGQGDSLHEHIANLAGSPAIKKGILQTVK | |
| | VVDELVKVMGRHKPENIVIEMARENQTTQKGQKNSRERMKRIEEGIKELGSQILKEHPVENTQLQNEK | |
| | LYLYYLQNGRDMYVDQELDINRLSDYDVDHIVPQSFLKDDSIDNKVLTRSDKNRGKSDNVPSEEVVKK | |
| | MKNYWRQLLNAKLITQRKFDNLTKAERGGLSELDKAGFIKRQLVETRQITKHVAQILDSRMNTKYDEN | |
| | DKLIREVKVITLKSKLVSDFRKDFQFYKVREINNYHHAHDAYLNAVVGTALIKKYPKLESEFVYGDYK | |
| | VYDVRKMIAKSEQEIGKATAKYFFYSNIMNFFKTEITLANGEIRKRPLIETNGETGEIVWDKGRDFAT | |
| | VRKVLSMPQVNIVKKTEVQTGGFSKESILPKRNSDKLIARKKDWDPKKYGGFVSPTVAYSVLVVAKVE | |
| | KGKSKKLKSVKELLGITIMERSSFEKNPIDFLEAKGYKEVKKDLIIKLPKYSLFELENGRKRMLASAR | |
| | ELQKGNELALPSKYVNFLYLASHYEKLKGSPEDNEQKQLFVEQHKHYLDEIIEQISEFSKRVILADAN | |
| | LDKVLSAYNKHRDKPIREQAENIIHLFTLTNLGAPAAFKYFDTTIDRKQYRSTKEVLDATLIHQSITG | |
| | LYETRIDLSQLGGDSGGSKRTADGSEFEPKKKRKV | |
| ABE8e | MKRTADGSEFESPKKKRKVSEVEFSHEYWMRHALTLAKRARDEREVPVGAVLVLNNRVIGEGWNRAIG | 202 |
| | LHDPTAHAEIMALRQGGLVMQNYRLIDATLYVTFEPCVMCAGAMIHSRIGRVVFGVRNSKRGAAGSLM | |
| | NVLNYPGMNHRVEITEGILADECAALLCDFYRMPRQVFNAQKKAQSSINSGGSSGGSSGSETPGTSES | |
| | ATPESSGGSSGGSDKKYSIGLAIGTNSVGWAVITDEYKVPSKKFKVLGNTDRHSIKKNLIGALLFDSG | |
| | ETAEATRLKRTARRRYTRRKNRICYLQEIFSNEMAKVDDSFFHRLEESFLVEEDKKHERHPIFGNIVD | |
| | EVAYHEKYPTIYHLRKKLVDSTDKADLRLIYLALAHMIKFRGHFLIEGDLNPDNSDVDKLFIQLVQTY | |
| | NQLFEENPINASGVDAKAILSARLSKSRRLENLIAQLPGEKKNGLFGNLIALSLGLTPNFKSNFDLAE | |
| | DAKLQLSKDTYDDDLDNLLAQIGDQYADLFLAAKNLSDAILLSDILRVNTEITKAPLSASMIKRYDEH | |
| | HQDLTLLKALVRQQLPEKYKEIFFDQSKNGYAGYIDGGASQEEFYKFIKPILEKMDGTEELLVKLNRE | |
| | DLLRKQRTFDNGSIPHQIHLGELHAILRRQEDFYPFLKDNREKIEKILTFRIPYYVGPLARGNSRFAW | |
| | MTRKSEETITPWNFEEVVDKGASAQSFIERMTNFDKNLPNEKVLPKHSLLYEYFTVYNELTKVKYVTE | |
| | GMRKPAFLSGEQKKAIVDLLFKTNRKVTVKQLKEDYFKKIECFDSVEISGVEDRFNASLGTYHDLLKI | |
| | IKDKDFLDNEENEDILEDIVLTLTLFEDREMIEERLKTYAHLFDDKVMKQLKRRRYTGWGRLSRKLIN | |
| | GIRDKQSGKTILDFLKSDGFANRNFMQLIHDDSLTFKEDIQKAQVSGQGDSLHEHIANLAGSPAIKKG | |
| | ILQTVKVVDELVKVMGRHKPENIVIEMARENQTTQKGQKNSRERMKRIEEGIKELGSQILKEHPVENT | |
| | QLQNEKLYLYYLQNGRDMYVDQELDINRLSDYDVDHIVPQSFLKDDSIDNKVLTRSDKNRGKSDNVPS | |
| | EEVVKKMKNYWRQLLNAKLITQRKFDNLTKAERGGLSELDKAGFIKRQLVETRQITKHVAQILDSRMN | |
| | TKYDENDKLIREVKVITLKSKLVSDFRKDFQFYKVREINNYHHAHDAYLNAVVGTALIKKYPKLESEF | |
| | VYGDYKVYDVRKMIAKSEQEIGKATAKYFFYSNIMNFFKTEITLANGEIRKRPLIETNGETGEIVWDK | |
| | GRDFATVRKVLSMPQVNIVKKTEVQTGGFSKESILPKRNSDKLIARKKDWDPKKYGGFDSPTVAYSVL | |
| | VVAKVEKGKSKKLKSVKELLGITIMERSSFEKNPIDFLEAKGYKEVKKDLIIKLPKYSLFELENGRKR | |
| | MLASAGELQKGNELALPSKYVNFLYLASHYEKLKGSPEDNEQKQLFVEQHKHYLDEIIEQISEFSKRV | |
| | ILADANLDKVLSAYNKHRDKPIREQAENIIHLFTLTNLGAPAAFKYFDTTIDRKRYTSTKEVLDATLI | |
| | HQSITGLYETRIDLSQLGGDSGGSKRTADGSEFEPKKKRKV | |
| ABE8e-dimer | MKRTADGSEFESPKKKRKVSEVEFSHEYWMRHALTLAKRAWDEREVPVGAVLVHNNRVIGEGWNRPIG | 203 |
| | RHDPTAHAEIMALRQGGLVMQNYRLIDATLYVTLEPCVMCAGAMIHSRIGRVVFGARDAKTGAAGSLM | |
| | DVLHHPGMNHRVEITEGILADECAALLSDFFRMRRQEIKAQKKAQSSTDSGGSSGGSSGSETPGTSES | |
| | ATPESSGGSSGGSSEVEFSHEYWMRHALTLAKRARDEREVPVGAVLVLNNRVIGEGWNRAIGLHDPTA | |
| | HAEIMALRQGGLVMQNYRLIDATLYVTFEPCVMCAGAMIHSRIGRVVFGVRNSKRGAAGSLMNVLNYP | |
| | GMNHRVEITEGILADECAALLCDFYRMPRQVFNAQKKAQSSINSGGSSGGSSGSETPGTSESATPESS | |
| | GGSSGGSDKKYSIGLAIGTNSVGWAVITDEYKVPSKKFKVLGNTDRHSIKKNLIGALLFDSGETAEAT | |
| | RLKRTARRRYTRRKNRICYLQEIFSNEMAKVDDSFFHRLEESFLVEEDKKHERHPIFGNIVDEVAYHE | |
| | KYPTIYHLRKKLVDSTDKADLRLIYLALAHMIKFRGHFLIEGDLNPDNSDVDKLFIQLVQTYNQLFEE | |
| | NPINASGVDAKAILSARLSKSRRLENLIAQLPGEKKNGLFGNLIALSLGLTPNFKSNFDLAEDAKLQL | |
| | SKDTYDDDLDNLLAQIGDQYADLFLAAKNLSDAILLSDILRVNTEITKAPLSASMIKRYDEHHQDLTL | |
| | LKALVRQQLPEKYKEIFFDQSKNGYAGYIDGGASQEEFYKFIKPILEKMDGTEELLVKLNREDLLRKQ | |
| | RTFDNGSIPHQIHLGELHAILRRQEDFYPFLKDNREKIEKILTFRIPYYVGPLARGNSRFAWMTRKSE | |
| | ETITPWNFEEVVDKGASAQSFIERMTNFDKNLPNEKVLPKHSLLYEYFTVYNELTKVKYVTEGMRKPA | |
| | FLSGEQKKAIVDLLFKTNRKVTVKQLKEDYFKKIECFDSVEISGVEDRFNASLGTYHDLLKIIKDKDF | |
| | LDNEENEDILEDIVLTLTLFEDREMIEERLKTYAHLFDDKVMKQLKRRRYTGWGRLSRKLINGIRDKQ | |
| | SGKTILDFLKSDGFANRNFMQLIHDDSLTFKEDIQKAQVSGQGDSLHEHIANLAGSPAIKKGILQTVK | |
| | VVDELVKVMGRHKPENIVIEMARENQTTQKGQKNSRERMKRIEEGIKELGSQILKEHPVENTQLQNEK | |
| | LYLYYLQNGRDMYVDQELDINRLSDYDVDHIVPQSFLKDDSIDNKVLTRSDKNRGKSDNVPSEEVVKK | |
| | MKNYWRQLLNAKLITQRKFDNLTKAERGGLSELDKAGFIKRQLVETRQITKHVAQILDSRMNTKYDEN | |
| | DKLIREVKVITLKSKLVSDFRKDFQFYKVREINNYHHAHDAYLNAVVGTALIKKYPKLESEFVYGDYK | |
| | VYDVRKMIAKSEQEIGKATAKYFFYSNIMNFFKTEITLANGEIRKRPLIETNGETGEIVWDKGRDFAT | |
| | VRKVLSMPQVNIVKKTEVQTGGFSKESILPKRNSDKLIARKKDWDPKKYGGFDSPTVAYSVLVVAKVE | |
| | KGKSKKLKSVKELLGITIMERSSFEKNPIDFLEAKGYKEVKKDLIIKLPKYSLFELENGRKRMLASAG | |
| | ELQKGNELALPSKYVNFLYLASHYEKLKGSPEDNEQKQLFVEQHKHYLDEIIEQISEFSKRVILADAN | |
| | LDKVLSAYNKHRDKPIREQAENIIHLFTLTNLGAPAAFKYFDTTIDRKRYTSTKEVLDATLIHQSITG | |
| | LYETRIDLSQLGGDSGGSKRTADGSEFEPKKKRKV | |
| SaABESe | MKRTADGSEFESPKKKRKVSEVEFSHEYWMRHALTLAKRARDEREVPVGAVLVLNNRVIGEGWNRAIG | 204 |
| | LHDPTAHAEIMALRQGGLVMQNYRLIDATLYVTFEPCVMCAGAMIHSRIGRVVFGVRNSKRGAAGSLM | |
| | NVLNYPGMNHRVEITEGILADECAALLCDFYRMPRQVFNAQKKAQSSINSGGSSGGSSGSETPGTSES | |
| | ATPESSGGSSGGSGKRNYILGLAIGITSVGYGIIDYETRDVIDAGVRLFKEANVENNEGRRSKRGARR | |
| | LKRRRRHRIQRVKKLLFDYNLLTDHSELSGINPYEARVKGLSQKLSEEEFSAALLHLAKRRGVHNVNE | |
| | VEEDTGNELSTKEQISRNSKALEEKYVAELQLERLKKDGEVRGSINRFKTSDYVKEAKQLLKVQKAYH | |
| | QLDQSFIDTYIDLLETRRTYYEGPGEGSPFGWKDIKEWYEMLMGHCTYFPEELRSVKYAYNADLYNAL | |
| | NDLNNLVITRDENEKLEYYEKFQIIENVFKQKKKPTLKQIAKEILVNEEDIKGYRVTSTGKPEFTNLK | |
| | VYHDIKDITARKEIIENAELLDQIAKILTIYQSSEDIQEELTNLNSELTQEEIEQISNLKGYTGTHNL | |
| | SLKAINLILDELWHTNDNQIAIFNRLKLVPKKVDLSQQKEIPTTLVDDFILSPVVKRSFIQSIKVINA | |
| | IIKKYGLPNDIIIELAREKNSKDAQKMINEMQKRNRQTNERIEEIIRTTGKENAKYLIEKIKLHDMQE | |
| | GKCLYSLEAIPLEDLLNNPFNYEVDHIIPRSVSFDNSFNNKVLVKQEENSKKGNRTPFQYLSSSDSKI | |
| | SYETFKKHILNLAKGKGRISKTKKEYLLEERDINRFSVQKDFINRNLVDTRYATRGLMNLLRSYFRVN | |
| | NLDVKVKSINGGFTSFLRRKWKFKKERNKGYKHHAEDALIIANADFIFKEWKKLDKAKKVMENQMFEE | |
| | KQAESMPEIETEQEYKEIFITPHQIKHIKDFKDYKYSHRVDKKPNRELINDTLYSTRKDDKGNTLIVN | |
| | NLNGLYDKDNDKLKKLINKSPEKLLMYHHDPQTYQKLKLIMEQYGDEKNPLYKYYEETGNYLTKYSKK | |
| | DNGPVIKKIKYYGNKLNAHLDITDDYPNSRNKVVKLSLKPYRFDVYLDNGVYKFVTVKNLDVIKKENY | |
| | YEVNSKCYEEAKKLKKISNQAEFIASFYNNDLIKINGELYRVIGVNNDLLNRIEVNMIDITYREYLEN | |
| | MNDKRPPRIIKTIASKTQSIKKYSTDILGNLYEVKSKKHPQIIKKGSGGSKRTADGSEFEPKKKRKV | |
| SaABESe-dimer | MKRTADGSEFESPKKKRKVSEVEFSHEYWMRHALTLAKRAWDEREVPVGAVLVHNNRVIGEGWNRPIG | 205 |
| | RHDPTAHAEIMALRQGGLVMQNYRLIDATLYVTLEPCVMCAGAMIHSRIGRVVFGARDAKTGAAGSLM | |
| | DVLHHPGMNHRVEITEGILADECAALLSDFFRMRRQEIKAQKKAQSSTDSGGSSGGSSGSETPGTSES | |
| | ATPESSGGSSGGSSEVEFSHEYWMRHALTLAKRARDEREVPVGAVLVLNNRVIGEGWNRAIGLHDPTA | |
| | HAEIMALRQGGLVMQNYRLIDATLYVTFEPCVMCAGAMIHSRIGRVVFGVRNSKRGAAGSLMNVLNYP | |
| | GMNHRVEITEGILADECAALLCDFYRMPRQVFNAQKKAQSSINSGGSSGGSSGSETPGTSESATPESS | |
| | GGSSGGSGKRNYILGLAIGITSVGYGIIDYETRDVIDAGVRLFKEANVENNEGRRSKRGARRLKRRRR | |
| | HRIQRVKKLLFDYNLLTDHSELSGINPYEARVKGLSQKLSEEEFSAALLHLAKRRGVHNVNEVEEDTG | |
| | NELSTKEQISRNSKALEEKYVAELQLERLKKDGEVRGSINRFKTSDYVKEAKQLLKVQKAYHQLDQSF | |
| | IDTYIDLLETRRTYYEGPGEGSPFGWKDIKEWYEMLMGHCTYFPEELRSVKYAYNADLYNALNDLNNL | |
| | VITRDENEKLEYYEKFQIIENVFKQKKKPTLKQIAKEILVNEEDIKGYRVTSTGKPEFTNLKVYHDIK | |
| | DITARKEIIENAELLDQIAKILTIYQSSEDIQEELTNLNSELTQEEIEQISNLKGYTGTHNLSLKAIN | |
| | LILDELWHTNDNQIAIFNRLKLVPKKVDLSQQKEIPTTLVDDFILSPVVKRSFIQSIKVINAIIKKYG | |
| | LPNDIIIELAREKNSKDAQKMINEMQKRNRQTNERIEEIIRTTGKENAKYLIEKIKLHDMQEGKCLYS | |
| | LEAIPLEDLLNNPFNYEVDHIIPRSVSFDNSFNNKVLVKQEENSKKGNRTPFQYLSSSDSKISYETFK | |
| | KHILNLAKGKGRISKTKKEYLLEERDINRFSVQKDFINRNLVDTRYATRGLMNLLRSYFRVNNLDVKV | |
| | KSINGGFTSFLRRKWKFKKERNKGYKHHAEDALIIANADFIFKEWKKLDKAKKVMENQMFEEKQAESM | |
| | PEIETEQEYKEIFITPHQIKHIKDFKDYKYSHRVDKKPNRELINDTLYSTRKDDKGNTLIVNNLNGLY | |
| | DKDNDKLKKLINKSPEKLLMYHHDPQTYQKLKLIMEQYGDEKNPLYKYYEETGNYLTKYSKKDNGPVI | |
| | KKIKYYGNKLNAHLDITDDYPNSRNKVVKLSLKPYRFDVYLDNGVYKFVTVKNLDVIKKENYYEVNSK | |
| | CYEEAKKLKKISNQAEFIASFYNNDLIKINGELYRVIGVNNDLLNRIEVNMIDITYREYLENMNDKRP | |
| | PRIIKTIASKTQSIKKYSTDILGNLYEVKSKKHPQIIKKGSGGSKRTADGSEFEPKKKRKV | |
| LbABEe | MKRTADGSEFESPKKKRKVSEVEFSHEYWMRHALTLAKRARDEREVPVGAVLVLNNRVIGEGWNRAIG | 206 |
| | LHDPTAHAEIMALRQGGLVMQNYRLIDATLYVTFEPCVMCAGAMIHSRIGRVVFGVRNSKRGAAGSLM | |
| | NVLNYPGMNHRVEITEGILADECAALLCDFYRMPRQVFNAQKKAQSSINSGGSSGGSSGSETPGTSES | |
| | ATPESSGGSSGGSSKLEKFTNCYSLSKTLRFKAIPVGKTQENIDNKRLLVEDEKRAEDYKGVKKLLDR | |
| | YYLSFINDVLHSIKLKNLNNYISLFRKKTRTEKENKELENLEINLRKEIAKAFKGNEGYKSLFKKDII | |
| | ETILPEFLDDKDEIALVNSFNGFTTAFTGFFDNRENMFSEEAKSTSIAFRCINENLTRYISNMDIFEK | |
| | VDAIFDKHEVQEIKEKILNSDYDVEDFFEGEFFNFVLTQEGIDVYNAIIGGFVTESGEKIKGLNEYIN | |
| | LYNQKTKQKLPKFKPLYKQVLSDRESLSFYGEGYTSDEEVLEVFRNTLNKNSEIFSSIKKLEKLFKNF | |
| | DEYSSAGIFVKNGPAISTISKDIFGEWNVIRDKWNAEYDDIHLKKKAVVTEKYEDDRRKSFKKIGSFS | |
| | LEQLQEYADADLSVVEKLKEIIIQKVDEIYKVYGSSEKLFDADFVLEKSLKKNDAVVAIMKDLLDSVK | |
| | SFENYIKAFFGEGKETNRDESFYGDFVLAYDILLKVDHIYDAIRNYVTQKPYSKDKFKLYFQNPQFMG | |
| | GWDKDKETDYRATILRYGSKYYLAIMDKKYAKCLQKIDKDDVNGNYEKINYKLLPGPNKMLPKVFFSK | |
| | KWMAYYNPSEDIQKIYKNGTFKKGDMFNLNDCHKLIDFFKDSISRYPKWSNAYDFNFSETEKYKDIAG | |
| | FYREVEEQGYKVSFESASKKEVDKLVEEGKLYMFQIYNKDFSDKSHGTPNLHTMYFKLLFDENNHGQI | |
| | RLSGGAELFMRRASLKKEELVVHPANSPIANKNPDNPKKTTTLSYDVYKDKRFSEDQYELHIPIAINK | |
| | CPKNIFKINTEVRVLLKHDDNPYVIGIARGERNLLYIVVVDGKGNIVEQYSLNEIINNFNGIRIKTDY | |
| | HSLLDKKEKERFEARQNWTSIENIKELKAGYISQVVHKICELVEKYDAVIALEDLNSGFKNSRVKVEK | |
| | QVYQKFEKMLIDKLNYMVDKKSNPCATGGALKGYQITNKFESFKSMSTQNGFIFYIPAWLTSKIDPST | |
| | GFVNLLKTKYTSIADSKKFISSFDRIMYVPEEDLFEFALDYKNFSRTDADYIKKWKLYSYGNRIRIFR | |
| | NPKKNNVFDWEEVCLTSAYKELFNKYGINYQQGDIRALLCEQSDKAFYSSFMALMSLMLQMRNSITGR | |
| | TDVDFLISPVKNSDGIFYDSRNYEAQENAILPKNADANGAYNIARKVLWAIGQFKKAEDEKLDKVKIA | |
| | ISNKEWLEYAQTSVKSGGSKRTADGSEFEPKKKRKV | |
| LbABE8e-dimer | MKRTADGSEFESPKKKRKVSEVEFSHEYWMRHALTLAKRAWDEREVPVGAVLVHNNRVIGEGWNRPIG | 207 |
| | RHDPTAHAEIMALRQGGLVMQNYRLIDATLYVTLEPCVMCAGAMIHSRIGRVVFGARDAKTGAAGSLM | |
| | DVLHHPGMNHRVEITEGILADECAALLSDFFRMRRQEIKAQKKAQSSTDSGGSSGGSSGSETPGTSES | |
| | ATPESSGGSSGGSSEVEFSHEYWMRHALTLAKRARDEREVPVGAVLVLNNRVIGEGWNRAIGLHDPTA | |
| | HAEIMALRQGGLVMQNYRLIDATLYVTFEPCVMCAGAMIHSRIGRVVFGVRNSKRGAAGSLMNVLNYP | |
| | GMNHRVEITEGILADECAALLCDFYRMPRQVFNAQKKAQSSINSGGSSGGSSGSETPGTSESATPESS | |
| | GGSSGGSSKLEKFTNCYSLSKTLRFKAIPVGKTQENIDNKRLLVEDEKRAEDYKGVKKLLDRYYLSFI | |
| | NDVLHSIKLKNLNNYISLFRKKTRTEKENKELENLEINLRKEIAKAFKGNEGYKSLFKKDIIETILPE | |
| | FLDDKDEIALVNSFNGFTTAFTGFFDNRENMFSEEAKSTSIAFRCINENLTRYISNMDIFEKVDAIFD | |
| | KHEVQEIKEKILNSDYDVEDFFEGEFFNFVLTQEGIDVYNAIIGGFVTESGEKIKGLNEYINLYNQKT | |
| | KQKLPKFKPLYKQVLSDRESLSFYGEGYTSDEEVLEVFRNTLNKNSEIFSSIKKLEKLFKNFDEYSSA | |
| | GIFVKNGPAISTISKDIFGEWNVIRDKWNAEYDDIHLKKKAVVTEKYEDDRRKSFKKIGSFSLEQLQE | |
| | YADADLSVVEKLKEIIIQKVDEIYKVYGSSEKLFDADFVLEKSLKKNDAVVAIMKDLLDSVKSFENYI | |
| | KAFFGEGKETNRDESFYGDFVLAYDILLKVDHIYDAIRNYVTQKPYSKDKFKLYFQNPQFMGGWDKDK | |
| | ETDYRATILRYGSKYYLAIMDKKYAKCLQKIDKDDVNGNYEKINYKLLPGPNKMLPKVFFSKKWMAYY | |
| | NPSEDIQKIYKNGTFKKGDMFNLNDCHKLIDFFKDSISRYPKWSNAYDFNFSETEKYKDIAGFYREVE | |
| | EQGYKVSFESASKKEVDKLVEEGKLYMFQIYNKDFSDKSHGTPNLHTMYFKLLFDENNHGQIRLSGGA | |
| | ELFMRRASLKKEELVVHPANSPIANKNPDNPKKTTTLSYDVYKDKRFSEDQYELHIPIAINKCPKNIF | |
| | KINTEVRVLLKHDDNPYVIGIARGERNLLYIVVVDGKGNIVEQYSLNEIINNFNGIRIKTDYHSLLDK | |
| | KEKERFEARQNWTSIENIKELKAGYISQVVHKICELVEKYDAVIALEDLNSGFKNSRVKVEKQVYQKF | |
| | EKMLIDKLNYMVDKKSNPCATGGALKGYQITNKFESFKSMSTQNGFIFYIPAWLTSKIDPSTGFVNLL | |
| | KTKYTSIADSKKFISSFDRIMYVPEEDLFEFALDYKNFSRTDADYIKKWKLYSYGNRIRIFRNPKKNN | |
| | VFDWEEVCLTSAYKELFNKYGINYQQGDIRALLCEQSDKAFYSSFMALMSLMLQMRNSITGRTDVDFL | |
| | ISPVKNSDGIFYDSRNYEAQENAILPKNADANGAYNIARKVLWAIGQFKKAEDEKLDKVKIAISNKEW | |
| | LEYAQTSVKSGGSKRTADGSEFEPKKKRKV | |
| LbABE7.10 | MKRTADGSEFESPKKKRKVSEVEFSHEYWMRHALTLAKRAWDEREVPVGAVLVHNNRVIGEGWNRPIG | 208 |
| | RHDPTAHAEIMALRQGGLVMQNYRLIDATLYVTLEPCVMCAGAMIHSRIGRVVFGARDAKTGAAGSLM | |
| | DVLHHPGMNHRVEITEGILADECAALLSDFFRMRRQEIKAQKKAQSSTDSGGSSGGSSGSETPGTSES | |
| | ATPESSGGSSGGSSEVEFSHEYWMRHALTLAKRARDEREVPVGAVLVLNNRVIGEGWNRAIGLHDPTA | |
| | HAEIMALRQGGLVMQNYRLIDATLYVTFEPCVMCAGAMIHSRIGRVVFGVRNAKTGAAGSLMDVLHYP | |
| | GMNHRVEITEGILADECAALLCYFFRMPRQVFNAQKKAQSSTDSGGSSGGSSGSETPGTSESATPESS | |
| | GGSSGGSSKLEKFTNCYSLSKTLRFKAIPVGKTQENIDNKRLLVEDEKRAEDYKGVKKLLDRYYLSFI | |
| | NDVLHSIKLKNLNNYISLFRKKTRTEKENKELENLEINLRKEIAKAFKGNEGYKSLFKKDIIETILPE | |
| | FLDDKDEIALVNSFNGFTTAFTGFFDNRENMFSEEAKSTSIAFRCINENLTRYISNMDIFEKVDAIFD | |
| | KHEVQEIKEKILNSDYDVEDFFEGEFFNFVLTQEGIDVYNAIIGGFVTESGEKIKGLNEYINLYNQKT | |
| | KQKLPKFKPLYKQVLSDRESLSFYGEGYTSDEEVLEVFRNTLNKNSEIFSSIKKLEKLFKNFDEYSSA | |
| | GIFVKNGPAISTISKDIFGEWNVIRDKWNAEYDDIHLKKKAVVTEKYEDDRRKSFKKIGSFSLEQLQE | |
| | YADADLSVVEKLKEIIIQKVDEIYKVYGSSEKLFDADFVLEKSLKKNDAVVAIMKDLLDSVKSFENYI | |
| | KAFFGEGKETNRDESFYGDFVLAYDILLKVDHIYDAIRNYVTQKPYSKDKFKLYFQNPQFMGGWDKDK | |
| | ETDYRATILRYGSKYYLAIMDKKYAKCLQKIDKDDVNGNYEKINYKLLPGPNKMLPKVFFSKKWMAYY | |
| | NPSEDIQKIYKNGTFKKGDMFNLNDCHKLIDFFKDSISRYPKWSNAYDFNFSETEKYKDIAGFYREVE | |
| | EQGYKVSFESASKKEVDKLVEEGKLYMFQIYNKDFSDKSHGTPNLHTMYFKLLFDENNHGQIRLSGGA | |
| | ELFMRRASLKKEELVVHPANSPIANKNPDNPKKTTTLSYDVYKDKRFSEDQYELHIPIAINKCPKNIF | |
| | KINTEVRVLLKHDDNPYVIGIARGERNLLYIVVVDGKGNIVEQYSLNEIINNFNGIRIKTDYHSLLDK | |
| | KEKERFEARQNWTSIENIKELKAGYISQVVHKICELVEKYDAVIALEDLNSGFKNSRVKVEKQVYQKF | |
| | EKMLIDKLNYMVDKKSNPCATGGALKGYQITNKFESFKSMSTQNGFIFYIPAWLTSKIDPSTGFVNLL | |
| | KTKYTSIADSKKFISSFDRIMYVPEEDLFEFALDYKNFSRTDADYIKKWKLYSYGNRIRIFRNPKKNN | |
| | VFDWEEVCLTSAYKELFNKYGINYQQGDIRALLCEQSDKAFYSSFMALMSLMLQMRNSITGRTDVDFL | |
| | ISPVKNSDGIFYDSRNYEAQENAILPKNADANGAYNIARKVLWAIGQFKKAEDEKLDKVKIAISNKEW | |
| | LEYAQTSVKSGGSKRTADGSEFEPKKKRKV | |
| enAsABE8e | MKRTADGSEFESPKKKRKVSEVEFSHEYWMRHALTLAKRARDEREVPVGAVLVLNNRVIGEGWNRAIG | 209 |
| | LHDPTAHAEIMALRQGGLVMQNYRLIDATLYVTFEPCVMCAGAMIHSRIGRVVFGVRNSKRGAAGSLM | |
| | NVLNYPGMNHRVEITEGILADECAALLCDFYRMPRQVFNAQKKAQSSINSGGSSGGSSGSETPGTSES | |
| | ATPESSGGSSGGSMTQFEGFTNLYQVSKTLRFELIPQGKTLKHIQEQGFIEEDKARNDHYKELKPIID | |
| | RIYKTYADQCLQLVQLDWENLSAAIDSYRKEKTEETRNALIEEQATYRNAIHDYFIGRTDNLTDAINK | |
| | RHAEIYKGLFKAELFNGKVLKQLGTVTTTEHENALLRSFDKFTTYFSGFYRNRKNVFSAEDISTAIPH | |
| | RIVQDNFPKFKENCHIFTRLITAVPSLREHFENVKKAIGIFVSTSIEEVFSFPFYNQLLTQTQIDLYN | |
| | QLLGGISREAGTEKIKGLNEVLNLAIQKNDETAHIIASLPHRFIPLFKQILSDRNTLSFILEEFKSDE | |
| | EVIQSFCKYKTLLRNENVLETAEALFNELNSIDLTHIFISHKKLETISSALCDHWDTLRNALYERRIS | |
| | ELTGKITKSAKEKVQRSLKHEDINLQEIISAAGKELSEAFKQKTSEILSHAHAALDQPLPTTLKKQEE | |
| | KEILKSQLDSLLGLYHLLDWFAVDESNEVDPEFSARLTGIKLEMEPSLSFYNKARNYATKKPYSVEKF | |
| | KLNFQMPTLARGWDVNREKNNGAILFVKNGLYYLGIMPKQKGRYKALSFEPTEKTSEGFDKMYYDYFP | |
| | DAAKMIPKCSTQLKAVTAHFQTHTTPILLSNNFIEPLEITKEIYDLNNPEKEPKKFQTAYAKKTGDQK | |
| | GYREALCKWIDFTRDFLSKYTKTTSIDLSSLRPSSQYKDLGEYYAELNPLLYHISFQRIAEKEIMDAV | |
| | ETGKLYLFQIYNKDFAKGHHGKPNLHTLYWTGLFSPENLAKTSIKLNGQAELFYRPKSRMKRMAHRLG | |
| | EKMLNKKLKDQKTPIPDTLYQELYDYVNHRLSHDLSDEARALLPNVITKEVSHEIIKDRRFTSDKFFF | |
| | HVPITLNYQAANSPSKFNQRVNAYLKEHPETPIIGIARGERNLIYITVIDSTGKILEQRSLNTIQQFD | |
| | YQKKLDNREKERVAARQAWSVVGTIKDLKQGYLSQVIHEIVDLMIHYQAVVVLENLNFGFKSKRTGIA | |
| | EKAVYQQFEKMLIDKLNCLVLKDYPAEKVGGVLNPYQLTDQFTSFAKMGTQSGFLFYVPAPYTSKIDP | |
| | LTGFVDPFVWKTIKNHESRKHFLEGFDFLHYDVKTGDFILHFKMNRNLSFQRGLPGFMPAWDIVFEKN | |
| | ETQFDAKGTPFIAGKRIVPVIENHRFTGRYRDLYPANELIALLEEKGIVFRDGSNILPKLLENDDSHA | |
| | IDTMVALIRSVLQMRNSNAATGEDYINSPVRDLNGVCFDSRFQNPEWPMDADANGAYHIALKGQLLLN | |
| | HLKESKDLKLQNGISNQDWLAYIQELRNSGGSKRTADGSEFEPKKKRKV | |
| enAsABE8e- | MKRTADGSEFESPKKKRKVSEVEFSHEYWMRHALTLAKRAWDEREVPVGAVLVHNNRVIGEGWNRPIG | 210 |
| dimer | RHDPTAHAEIMALRQGGLVMQNYRLIDATLYVTLEPCVMCAGAMIHSRIGRVVFGARDAKTGAAGSLM | |
| | DVLHHPGMNHRVEITEGILADECAALLSDFFRMRRQEIKAQKKAQSSTDSGGSSGGSSGSETPGTSES | |
| | ATPESSGGSSGGSSEVEFSHEYWMRHALTLAKRARDEREVPVGAVLVLNNRVIGEGWNRAIGLHDPTA | |
| | HAEIMALRQGGLVMQNYRLIDATLYVTFEPCVMCAGAMIHSRIGRVVFGVRNSKRGAAGSLMNVLNYP | |
| | GMNHRVEITEGILADECAALLCDFYRMPRQVFNAQKKAQSSINSGGSSGGSSGSETPGTSESATPESS | |
| | GGSSGGSMTQFEGFTNLYQVSKTLRFELIPQGKTLKHIQEQGFIEEDKARNDHYKELKPIIDRIYKTY | |
| | ADQCLQLVQLDWENLSAAIDSYRKEKTEETRNALIEEQATYRNAIHDYFIGRTDNLTDAINKRHAEIY | |
| | KGLFKAELFNGKVLKQLGTVTTTEHENALLRSFDKFTTYFSGFYRNRKNVFSAEDISTAIPHRIVQDN | |
| | FPKFKENCHIFTRLITAVPSLREHFENVKKAIGIFVSTSIEEVFSFPFYNQLLTQTQIDLYNQLLGGI | |
| | SREAGTEKIKGLNEVLNLAIQKNDETAHIIASLPHRFIPLFKQILSDRNTLSFILEEFKSDEEVIQSF | |
| | CKYKTLLRNENVLETAEALFNELNSIDLTHIFISHKKLETISSALCDHWDTLRNALYERRISELTGKI | |
| | TKSAKEKVQRSLKHEDINLQEIISAAGKELSEAFKQKTSEILSHAHAALDQPLPTTLKKQEEKEILKS | |
| | QLDSLLGLYHLLDWFAVDESNEVDPEFSARLTGIKLEMEPSLSFYNKARNYATKKPYSVEKFKLNFQM | |
| | PTLARGWDVNREKNNGAILFVKNGLYYLGIMPKQKGRYKALSFEPTEKTSEGFDKMYYDYFPDAAKMI | |
| | PKCSTQLKAVTAHFQTHTTPILLSNNFIEPLEITKEIYDLNNPEKEPKKFQTAYAKKTGDQKGYREAL | |
| | CKWIDFTRDFLSKYTKTTSIDLSSLRPSSQYKDLGEYYAELNPLLYHISFQRIAEKEIMDAVETGKLY | |
| | LFQIYNKDFAKGHHGKPNLHTLYWTGLFSPENLAKTSIKLNGQAELFYRPKSRMKRMAHRLGEKMLNK | |
| | KLKDQKTPIPDTLYQELYDYVNHRLSHDLSDEARALLPNVITKEVSHEIIKDRRFTSDKFFFHVPITL | |
| | NYQAANSPSKFNQRVNAYLKEHPETPIIGIARGERNLIYITVIDSTGKILEQRSLNTIQQFDYQKKLD | |
| | NREKERVAARQAWSVVGTIKDLKQGYLSQVIHEIVDLMIHYQAVVVLENLNFGFKSKRTGIAEKAVYQ | |
| | QFEKMLIDKLNCLVLKDYPAEKVGGVLNPYQLTDQFTSFAKMGTQSGFLFYVPAPYTSKIDPLTGFVD | |
| | PFVWKTIKNHESRKHFLEGFDFLHYDVKTGDFILHFKMNRNLSFQRGLPGFMPAWDIVFEKNETQFDA | |
| | KGTPFIAGKRIVPVIENHRFTGRYRDLYPANELIALLEEKGIVFRDGSNILPKLLENDDSHAIDTMVA | |
| | LIRSVLQMRNSNAATGEDYINSPVRDLNGVCFDSRFQNPEWPMDADANGAYHIALKGQLLLNHLKESK | |
| | DLKLQNGISNQDWLAYIQELRNSGGSKRTADGSEFEPKKKRKV | |
| enAsABE7 . 10 | MKRTADGSEFESPKKKRKVSEVEFSHEYWMRHALTLAKRAWDEREVPVGAVLVHNNRVIGEGWNRPIG | 211 |
| | RHDPTAHAEIMALRQGGLVMQNYRLIDATLYVTLEPCVMCAGAMIHSRIGRVVFGARDAKTGAAGSLM | |
| | DVLHHPGMNHRVEITEGILADECAALLSDFFRMRRQEIKAQKKAQSSTDSGGSSGGSSGSETPGTSES | |
| | ATPESSGGSSGGSSEVEFSHEYWMRHALTLAKRARDEREVPVGAVLVLNNRVIGEGWNRAIGLHDPTA | |
| | HAEIMALRQGGLVMQNYRLIDATLYVTFEPCVMCAGAMIHSRIGRVVFGVRNAKTGAAGSLMDVLHYP | |
| | GMNHRVEITEGILADECAALLCYFFRMPRQVFNAQKKAQSSTDSGGSSGGSSGSETPGTSESATPESS | |
| | GGSSGGSMTQFEGFTNLYQVSKTLRFELIPQGKTLKHIQEQGFIEEDKARNDHYKELKPIIDRIYKTY | |
| | ADQCLQLVQLDWENLSAAIDSYRKEKTEETRNALIEEQATYRNAIHDYFIGRTDNLTDAINKRHAEIY | |
| | KGLFKAELFNGKVLKQLGTVTTTEHENALLRSFDKFTTYFSGFYRNRKNVFSAEDISTAIPHRIVQDN | |
| | FPKFKENCHIFTRLITAVPSLREHFENVKKAIGIFVSTSIEEVFSFPFYNQLLTQTQIDLYNQLLGGI | |
| | SREAGTEKIKGLNEVLNLAIQKNDETAHIIASLPHRFIPLFKQILSDRNTLSFILEEFKSDEEVIQSF | |
| | CKYKTLLRNENVLETAEALFNELNSIDLTHIFISHKKLETISSALCDHWDTLRNALYERRISELTGKI | |
| | TKSAKEKVQRSLKHEDINLQEIISAAGKELSEAFKQKTSEILSHAHAALDQPLPTTLKKQEEKEILKS | |
| | QLDSLLGLYHLLDWFAVDESNEVDPEFSARLTGIKLEMEPSLSFYNKARNYATKKPYSVEKFKLNFQM | |
| | PTLARGWDVNREKNNGAILFVKNGLYYLGIMPKQKGRYKALSFEPTEKTSEGFDKMYYDYFPDAAKMI | |
| | PKCSTQLKAVTAHFQTHTTPILLSNNFIEPLEITKEIYDLNNPEKEPKKFQTAYAKKTGDQKGYREAL | |
| | CKWIDFTRDFLSKYTKTTSIDLSSLRPSSQYKDLGEYYAELNPLLYHISFQRIAEKEIMDAVETGKLY | |
| | LFQIYNKDFAKGHHGKPNLHTLYWTGLFSPENLAKTSIKLNGQAELFYRPKSRMKRMAHRLGEKMLNK | |
| | KLKDQKTPIPDTLYQELYDYVNHRLSHDLSDEARALLPNVITKEVSHEIIKDRRFTSDKFFFHVPITL | |
| | NYQAANSPSKFNQRVNAYLKEHPETPIIGIARGERNLIYITVIDSTGKILEQRSLNTIQQFDYQKKLD | |
| | NREKERVAARQAWSVVGTIKDLKQGYLSQVIHEIVDLMIHYQAVVVLENLNFGFKSKRTGIAEKAVYQ | |
| | QFEKMLIDKLNCLVLKDYPAEKVGGVLNPYQLTDQFTSFAKMGTQSGFLFYVPAPYTSKIDPLTGFVD | |
| | PFVWKTIKNHESRKHFLEGFDFLHYDVKTGDFILHFKMNRNLSFQRGLPGFMPAWDIVFEKNETQFDA | |
| | KGTPFIAGKRIVPVIENHRFTGRYRDLYPANELIALLEEKGIVFRDGSNILPKLLENDDSHAIDTMVA | |
| | LIRSVLQMRNSNAATGEDYINSPVRDLNGVCFDSRFQNPEWPMDADANGAYHIALKGQLLLNHLKESK | |
| | DLKLQNGISNQDWLAYIQELRNSGGSKRTADGSEFEPKKKRKV | |
| SpCas9NG-ABE8e | MKRTADGSEFESPKKKRKVSEVEFSHEYWMRHALTLAKRARDEREVPVGAVLVLNNRVIGEGWNRAIG | 212 |
| (″NG-ABE8e″) | LHDPTAHAEIMALRQGGLVMQNYRLIDATLYVTFEPCVMCAGAMIHSRIGRVVFGVRNSKRGAAGSLM | |
| | NVLNYPGMNHRVEITEGILADECAALLCDFYRMPRQVFNAQKKAQSSINSGGSSGGSSGSETPGTSES | |
| | ATPESSGGSSGGSDKKYSIGLAIGTNSVGWAVITDEYKVPSKKFKVLGNTDRHSIKKNLIGALLFDSG | |
| | ETAEATRLKRTARRRYTRRKNRICYLQEIFSNEMAKVDDSFFHRLEESFLVEEDKKHERHPIFGNIVD | |
| | EVAYHEKYPTIYHLRKKLVDSTDKADLRLIYLALAHMIKFRGHFLIEGDLNPDNSDVDKLFIQLVQTY | |
| | NQLFEENPINASGVDAKAILSARLSKSRRLENLIAQLPGEKKNGLFGNLIALSLGLTPNFKSNFDLAE | |
| | DAKLQLSKDTYDDDLDNLLAQIGDQYADLFLAAKNLSDAILLSDILRVNTEITKAPLSASMIKRYDEH | |
| | HQDLTLLKALVRQQLPEKYKEIFFDQSKNGYAGYIDGGASQEEFYKFIKPILEKMDGTEELLVKLNRE | |
| | DLLRKQRTFDNGSIPHQIHLGELHAILRRQEDFYPFLKDNREKIEKILTFRIPYYVGPLARGNSRFAW | |
| | MTRKSEETITPWNFEEVVDKGASAQSFIERMTNFDKNLPNEKVLPKHSLLYEYFTVYNELTKVKYVTE | |
| | GMRKPAFLSGEQKKAIVDLLFKTNRKVTVKQLKEDYFKKIECFDSVEISGVEDRFNASLGTYHDLLKI | |
| | IKDKDFLDNEENEDILEDIVLTLTLFEDREMIEERLKTYAHLFDDKVMKQLKRRRYTGWGRLSRKLIN | |
| | GIRDKQSGKTILDFLKSDGFANRNFMQLIHDDSLTFKEDIQKAQVSGQGDSLHEHIANLAGSPAIKKG | |
| | ILQTVKVVDELVKVMGRHKPENIVIEMARENQTTQKGQKNSRERMKRIEEGIKELGSQILKEHPVENT | |
| | QLQNEKLYLYYLQNGRDMYVDQELDINRLSDYDVDHIVPQSFLKDDSIDNKVLTRSDKNRGKSDNVPS | |
| | EEVVKKMKNYWRQLLNAKLITQRKFDNLTKAERGGLSELDKAGFIKRQLVETRQITKHVAQILDSRMN | |
| | TKYDENDKLIREVKVITLKSKLVSDFRKDFQFYKVREINNYHHAHDAYLNAVVGTALIKKYPKLESEF | |
| | VYGDYKVYDVRKMIAKSEQEIGKATAKYFFYSNIMNFFKTEITLANGEIRKRPLIETNGETGEIVWDK | |
| | GRDFATVRKVLSMPQVNIVKKTEVQTGGFSKESIRPKRNSDKLIARKKDWDPKKYGGFVSPTVAYSVL | |
| | VVAKVEKGKSKKLKSVKELLGITIMERSSFEKNPIDFLEAKGYKEVKKDLIIKLPKYSLFELENGRKR | |
| | MLASARFLQKGNELALPSKYVNFLYLASHYEKLKGSPEDNEQKQLFVEQHKHYLDEIIEQISEFSKRV | |
| | ILADANLDKVLSAYNKHRDKPIREQAENIIHLFTLTNLGAPRAFKYFDTTIDRKVYRSTKEVLDATLI | |
| | HQSITGLYETRIDLSQLGGDSGGSKRTADGSEFEPKKKRKV | |
| NG-ABE8e-dimer | MKRTADGSEFESPKKKRKVSEVEFSHEYWMRHALTLAKRAWDEREVPVGAVLVHNNRVIGEGWNRPIG | 213 |
| | RHDPTAHAEIMALRQGGLVMQNYRLIDATLYVTLEPCVMCAGAMIHSRIGRVVFGARDAKTGAAGSLM | |
| | DVLHHPGMNHRVEITEGILADECAALLSDFFRMRRQEIKAQKKAQSSTDSGGSSGGSSGSETPGTSES | |
| | ATPESSGGSSGGSSEVEFSHEYWMRHALTLAKRARDEREVPVGAVLVLNNRVIGEGWNRAIGLHDPTA | |
| | HAEIMALRQGGLVMQNYRLIDATLYVTFEPCVMCAGAMIHSRIGRVVFGVRNSKRGAAGSLMNVLNYP | |
| | GMNHRVEITEGILADECAALLCDFYRMPRQVFNAQKKAQSSINSGGSSGGSSGSETPGTSESATPESS | |
| | GGSSGGSDKKYSIGLAIGTNSVGWAVITDEYKVPSKKFKVLGNTDRHSIKKNLIGALLFDSGETAEAT | |
| | RLKRTARRRYTRRKNRICYLQEIFSNEMAKVDDSFFHRLEESFLVEEDKKHERHPIFGNIVDEVAYHE | |
| | KYPTIYHLRKKLVDSTDKADLRLIYLALAHMIKFRGHFLIEGDLNPDNSDVDKLFIQLVQTYNQLFEE | |
| | NPINASGVDAKAILSARLSKSRRLENLIAQLPGEKKNGLFGNLIALSLGLTPNFKSNFDLAEDAKLQL | |
| | SKDTYDDDLDNLLAQIGDQYADLFLAAKNLSDAILLSDILRVNTEITKAPLSASMIKRYDEHHQDLTL | |
| | LKALVRQQLPEKYKEIFFDQSKNGYAGYIDGGASQEEFYKFIKPILEKMDGTEELLVKLNREDLLRKQ | |
| | RTFDNGSIPHQIHLGELHAILRRQEDFYPFLKDNREKIEKILTFRIPYYVGPLARGNSRFAWMTRKSE | |
| | ETITPWNFEEVVDKGASAQSFIERMTNFDKNLPNEKVLPKHSLLYEYFTVYNELTKVKYVTEGMRKPA | |
| | FLSGEQKKAIVDLLFKTNRKVTVKQLKEDYFKKIECFDSVEISGVEDRFNASLGTYHDLLKIIKDKDF | |
| | LDNEENEDILEDIVLTLTLFEDREMIEERLKTYAHLFDDKVMKQLKRRRYTGWGRLSRKLINGIRDKQ | |
| | SGKTILDFLKSDGFANRNFMQLIHDDSLTFKEDIQKAQVSGQGDSLHEHIANLAGSPAIKKGILQTVK | |
| | VVDELVKVMGRHKPENIVIEMARENQTTQKGQKNSRERMKRIEEGIKELGSQILKEHPVENTQLQNEK | |
| | LYLYYLQNGRDMYVDQELDINRLSDYDVDHIVPQSFLKDDSIDNKVLTRSDKNRGKSDNVPSEEVVKK | |
| | MKNYWRQLLNAKLITQRKFDNLTKAERGGLSELDKAGFIKRQLVETRQITKHVAQILDSRMNTKYDEN | |
| | DKLIREVKVITLKSKLVSDFRKDFQFYKVREINNYHHAHDAYLNAVVGTALIKKYPKLESEFVYGDYK | |
| | VYDVRKMIAKSEQEIGKATAKYFFYSNIMNFFKTEITLANGEIRKRPLIETNGETGEIVWDKGRDFAT | |
| | VRKVLSMPQVNIVKKTEVQTGGFSKESIRPKRNSDKLIARKKDWDPKKYGGFVSPTVAYSVLVVAKVE | |
| | KGKSKKLKSVKELLGITIMERSSFEKNPIDFLEAKGYKEVKKDLIIKLPKYSLFELENGRKRMLASAR | |
| | FLQKGNELALPSKYVNFLYLASHYEKLKGSPEDNEQKQLFVEQHKHYLDEIIEQISEFSKRVILADAN | |
| | LDKVLSAYNKHRDKPIREQAENIIHLFTLTNLGAPRAFKYFDTTIDRKVYRSTKEVLDATLIHQSITG | |
| | LYETRIDLSQLGGDSGGSKRTADGSEFEPKKKRKV | |
| SaKKH-ABEe | MKRTADGSEFESPKKKRKVSEVEFSHEYWMRHALTLAKRARDEREVPVGAVLVLNNRVIGEGWNRAIG | 214 |
| (″KKH-ABE8e″) | LHDPTAHAEIMALRQGGLVMQNYRLIDATLYVTFEPCVMCAGAMIHSRIGRVVFGVRNSKRGAAGSLM | |
| | NVLNYPGMNHRVEITEGILADECAALLCDFYRMPRQVFNAQKKAQSSINSGGSSGGSSGSETPGTSES | |
| | ATPESSGGSSGGSGKRNYILGLAIGITSVGYGIIDYETRDVIDAGVRLFKEANVENNEGRRSKRGARR | |
| | LKRRRRHRIQRVKKLLFDYNLLTDHSELSGINPYEARVKGLSQKLSEEEFSAALLHLAKRRGVHNVNE | |
| | VEEDTGNELSTKEQISRNSKALEEKYVAELQLERLKKDGEVRGSINRFKTSDYVKEAKQLLKVQKAYH | |
| | QLDQSFIDTYIDLLETRRTYYEGPGEGSPFGWKDIKEWYEMLMGHCTYFPEELRSVKYAYNADLYNAL | |
| | NDLNNLVITRDENEKLEYYEKFQIIENVFKQKKKPTLKQIAKEILVNEEDIKGYRVTSTGKPEFTNLK | |
| | VYHDIKDITARKEIIENAELLDQIAKILTIYQSSEDIQEELTNLNSELTQEEIEQISNLKGYTGTHNL | |
| | SLKAINLILDELWHTNDNQIAI FNRLKLVPKKVDLSQQKEIPTTLVDDFILSPWKRSFIQSIKVINA | |
| | IIKKYGLPNDIIIELAREKNSKDAQKMINEMQKRNRQTNERIEEIIRTTGKENAKYLIEKIKLHDMQE | |
| | GKCLYSLEAIPLEDLLNNPFNYEVDHIIPRSVSFDNSFNNKVLVKQEENSKKGNRTPFQYLSSSDSKI | |
| | SYETFKKHILNLAKGKGRISKTKKEYLLEERDINRFSVQKDFINRNLVDTRYATRGLMNLLRSYFRVN | |
| | NLDVKVKSINGGFTSFLRRKWKFKKERNKGYKHHAEDALIIANADFIFKEWKKLDKAKKVMENQMFEE | |
| | KQAESMPEIETEQEYKEIFITPHQIKHIKDFKDYKYSHRVDKKPNRELINDTLYSTRKDDKGNTLIVN | |
| | NLNGLYDKDNDKLKKLINKSPEKLLMYHHDPQTYQKLKLIMEQYGDEKNPLYKYYEETGNYLTKYSKK | |
| | DNGPVIKKIKYYGNKLNAHLDITDDYPNSRNKVVKLSLKPYRFDVYLDNGVYKFVTVKNLDVIKKENY | |
| | YEVNSKCYEEAKKLKKISNQAEFIASFYNNDLIKINGELYRVIGVNNDLLNRIEVNMIDITYREYLEN | |
| | MNDKRPPRIIKTIASKTQSIKKYSTDILGNLYEVKSKKHPQIIKKGSGGSKRTADGSEFEPKKKRKV | |
| SaKKH-ABE8e- | MKRTADGSEFESPKKKRKVSEVEFSHEYWMRHALTLAKRAWDEREVPVGAVLVHNNRVIGEGWNRPIG | 215 |
| dimer | RHDPTAHAEIMALRQGGLVMQNYRLIDATLYVTLEPCVMCAGAMIHSRIGRVVFGARDAKTGAAGSLM | |
| | DVLHHPGMNHRVEITEGILADECAALLSDFFRMRRQEIKAQKKAQSSTDSGGSSGGSSGSETPGTSES | |
| | ATPESSGGSSGGSSEVEFSHEYWMRHALTLAKRARDEREVPVGAVLVLNNRVIGEGWNRAIGLHDPTA | |
| | HAEIMALRQGGLVMQNYRLIDATLYVTFEPCVMCAGAMIHSRIGRVVFGVRNSKRGAAGSLMNVLNYP | |
| | GMNHRVEITEGILADECAALLCDFYRMPRQVFNAQKKAQSSINSGGSSGGSSGSETPGTSESATPESS | |
| | GGSSGGSGKRNYILGLAIGITSVGYGIIDYETRDVIDAGVRLFKEANVENNEGRRSKRGARRLKRRRR | |
| | HRIQRVKKLLFDYNLLTDHSELSGINPYEARVKGLSQKLSEEEFSAALLHLAKRRGVHNVNEVEEDTG | |
| | NELSTKEQISRNSKALEEKYVAELQLERLKKDGEVRGSINRFKTSDYVKEAKQLLKVQKAYHQLDQSF | |
| | IDTYIDLLETRRTYYEGPGEGSPFGWKDIKEWYEMLMGHCTYFPEELRSVKYAYNADLYNALNDLNNL | |
| | VITRDENEKLEYYEKFQIIENVFKQKKKPTLKQIAKEILVNEEDIKGYRVTSTGKPEFTNLKVYHDIK | |
| | DITARKEIIENAELLDQIAKILTIYQSSEDIQEELTNLNSELTQEEIEQISNLKGYTGTHNLSLKAIN | |
| | LILDELWHTNDNQIAIFNRLKLVPKKVDLSQQKEIPTTLVDDFILSPVVKRSFIQSIKVINAIIKKYG | |
| | LPNDIIIELAREKNSKDAQKMINEMQKRNRQTNERIEEIIRTTGKENAKYLIEKIKLHDMQEGKCLYS | |
| | LEAIPLEDLLNNPFNYEVDHIIPRSVSFDNSFNNKVLVKQEENSKKGNRTPFQYLSSSDSKISYETFK | |
| | KHILNLAKGKGRISKTKKEYLLEERDINRFSVQKDFINRNLVDTRYATRGLMNLLRSYFRVNNLDVKV | |
| | KSINGGFTSFLRRKWKFKKERNKGYKHHAEDALIIANADFIFKEWKKLDKAKKVMENQMFEEKQAESM | |
| | PEIETEQEYKEIFITPHQIKHIKDFKDYKYSHRVDKKPNRELINDTLYSTRKDDKGNTLIVNNLNGLY | |
| | DKDNDKLKKLINKSPEKLLMYHHDPQTYQKLKLIMEQYGDEKNPLYKYYEETGNYLTKYSKKDNGPVI | |
| | KKIKYYGNKLNAHLDITDDYPNSRNKVVKLSLKPYRFDVYLDNGVYKFVTVKNLDVIKKENYYEVNSK | |
| | CYEEAKKLKKISNQAEFIASFYNNDLIKINGELYRVIGVNNDLLNRIEVNMIDITYREYLENMNDKRP | |
| | PRIIKTIASKTQSIKKYSTDILGNLYEVKSKKHPQIIKKGSGGSKRTADGSEFEPKKKRKV | |
| CP1028-ABE8e | MKRTADGSEFESPKKKRKVSEVEFSHEYWMRHALTLAKRARDEREVPVGAVLVLNNRVIGEGWNRAIG | 216 |
| | LHDPTAHAEIMALRQGGLVMQNYRLIDATLYVTFEPCVMCAGAMIHSRIGRVVFGVRNSKRGAAGSLM | |
| | NVLNYPGMNHRVEITEGILADECAALLCDFYRMPRQVFNAQKKAQSSINSGGSSGGSSGSETPGTSES | |
| | ATPESSGGSSGGSEIGKATAKYFFYSNIMNFFKTEITLANGEIRKRPLIETNGETGEIVWDKGRDFAT | |
| | VRKVLSMPQVNIVKKTEVQTGGFSKESILPKRNSDKLIARKKDWDPKKYGGFDSPTVAYSVLVVAKVE | |
| | KGKSKKLKSVKELLGITIMERSSFEKNPIDFLEAKGYKEVKKDLIIKLPKYSLFELENGRKRMLASAG | |
| | ELQKGNELALPSKYVNFLYLASHYEKLKGSPEDNEQKQLFVEQHKHYLDEIIEQISEFSKRVILADAN | |
| | LDKVLSAYNKHRDKPIREQAENIIHLFTLTNLGAPAAFKYFDTTIDRKRYTSTKEVLDATLIHQSITG | |
| | LYETRIDLSQLGGDGGSGGSGGSGGSGGSGGSGGMDKKYSIGLAIGTNSVGWAVITDEYKVPSKKFKV | |
| | LGNTDRHSIKKNLIGALLFDSGETAEATRLKRTARRRYTRRKNRICYLQEIFSNEMAKVDDSFFHRLE | |
| | ESFLVEEDKKHERHPIFGNIVDEVAYHEKYPTIYHLRKKLVDSTDKADLRLIYLALAHMIKFRGHFLI | |
| | EGDLNPDNSDVDKLFIQLVQTYNQLFEENPINASGVDAKAILSARLSKSRRLENLIAQLPGEKKNGLF | |
| | GNLIALSLGLTPNFKSNFDLAEDAKLQLSKDTYDDDLDNLLAQIGDQYADLFLAAKNLSDAILLSDIL | |
| | RVNTEITKAPLSASMIKRYDEHHQDLTLLKALVRQQLPEKYKEIFFDQSKNGYAGYIDGGASQEEFYK | |
| | FIKPILEKMDGTEELLVKLNREDLLRKQRTFDNGSIPHQIHLGELHAILRRQEDFYPFLKDNREKIEK | |
| | ILTFRIPYYVGPLARGNSRFAWMTRKSEETITPWNFEEVVDKGASAQSFIERMTNFDKNLPNEKVLPK | |
| | HSLLYEYFTVYNELTKVKYVTEGMRKPAFLSGEQKKAIVDLLFKTNRKVTVKQLKEDYFKKIECFDSV | |
| | EISGVEDRFNASLGTYHDLLKIIKDKDFLDNEENEDILEDIVLTLTLFEDREMIEERLKTYAHLFDDK | |
| | VMKQLKRRRYTGWGRLSRKLINGIRDKQSGKTILDFLKSDGFANRNFMQLIHDDSLTFKEDIQKAQVS | |
| | GQGDSLHEHIANLAGSPAIKKGILQTVKVVDELVKVMGRHKPENIVIEMARENQTTQKGQKNSRERMK | |
| | RIEEGIKELGSQILKEHPVENTQLQNEKLYLYYLQNGRDMYVDQELDINRLSDYDVDHIVPQSFLKDD | |
| | SIDNKVLTRSDKNRGKSDNVPSEEVVKKMKNYWRQLLNAKLITQRKFDNLTKAERGGLSELDKAGFIK | |
| | RQLVETRQITKHVAQILDSRMNTKYDENDKLIREVKVITLKSKLVSDFRKDFQFYKVREINNYHHAHD | |
| | AYLNAVVGTALIKKYPKLESEFVYGDYKVYDVRKMIAKSEQSGGSKRTADGSEFEPKKKRKV | |
| CP1028-ABE8e- | MKRTADGSEFESPKKKRKVSEVEFSHEYWMRHALTLAKRAWDEREVPVGAVLVHNNRVIGEGWNRPIG | 217 |
| dimer | RHDPTAHAEIMALRQGGLVMQNYRLIDATLYVTLEPCVMCAGAMIHSRIGRVVFGARDAKTGAAGSLM | |
| | DVLHHPGMNHRVEITEGILADECAALLSDFFRMRRQEIKAQKKAQSSTDSGGSSGGSSGSETPGTSES | |
| | ATPESSGGSSGGSSEVEFSHEYWMRHALTLAKRARDEREVPVGAVLVLNNRVIGEGWNRAIGLHDPTA | |
| | HAEIMALRQGGLVMQNYRLIDATLYVTFEPCVMCAGAMIHSRIGRVVFGVRNSKRGAAGSLMNVLNYP | |
| | GMNHRVEITEGILADECAALLCDFYRMPRQVFNAQKKAQSSINSGGSSGGSSGSETPGTSESATPESS | |
| | GGSSGGSEIGKATAKYFFYSNIMNFFKTEITLANGEIRKRPLIETNGETGEIVWDKGRDFATVRKVLS | |
| | MPQVNIVKKTEVQTGGFSKESILPKRNSDKLIARKKDWDPKKYGGFDSPTVAYSVLVVAKVEKGKSKK | |
| | LKSVKELLGITIMERSSFEKNPIDFLEAKGYKEVKKDLIIKLPKYSLFELENGRKRMLASAGELQKGN | |
| | ELALPSKYVNFLYLASHYEKLKGSPEDNEQKQLFVEQHKHYLDEIIEQISEFSKRVILADANLDKVLS | |
| | AYNKHRDKPIREQAENIIHLFTLTNLGAPAAFKYFDTTIDRKRYTSTKEVLDATLIHQSITGLYETRI | |
| | DLSQLGGDGGSGGSGGSGGSGGSGGSGGMDKKYSIGLAIGTNSVGWAVITDEYKVPSKKFKVLGNTDR | |
| | HSIKKNLIGALLFDSGETAEATRLKRTARRRYTRRKNRICYLQEIFSNEMAKVDDSFFHRLEESFLVE | |
| | EDKKHERHPIFGNIVDEVAYHEKYPTIYHLRKKLVDSTDKADLRLIYLALAHMIKFRGHFLIEGDLNP | |
| | DNSDVDKLFIQLVQTYNQLFEENPINASGVDAKAILSARLSKSRRLENLIAQLPGEKKNGLFGNLIAL | |
| | SLGLTPNFKSNFDLAEDAKLQLSKDTYDDDLDNLLAQIGDQYADLFLAAKNLSDAILLSDILRVNTEI | |
| | TKAPLSASMIKRYDEHHQDLTLLKALVRQQLPEKYKEIFFDQSKNGYAGYIDGGASQEEFYKFIKPIL | |
| | EKMDGTEELLVKLNREDLLRKQRTFDNGSIPHQIHLGELHAILRRQEDFYPFLKDNREKIEKILTFRI | |
| | PYYVGPLARGNSRFAWMTRKSEETITPWNFEEVVDKGASAQSFIERMTNFDKNLPNEKVLPKHSLLYE | |
| | YFTVYNELTKVKYVTEGMRKPAFLSGEQKKAIVDLLFKTNRKVTVKQLKEDYFKKIECFDSVEISGVE | |
| | DRFNASLGTYHDLLKIIKDKDFLDNEENEDILEDIVLTLTLFEDREMIEERLKTYAHLFDDKVMKQLK | |
| | RRRYTGWGRLSRKLINGIRDKQSGKTILDFLKSDGFANRNFMQLIHDDSLTFKEDIQKAQVSGQGDSL | |
| | HEHIANLAGSPAIKKGILQTVKVVDELVKVMGRHKPENIVIEMARENQTTQKGQKNSRERMKRIEEGI | |
| | KELGSQILKEHPVENTQLQNEKLYLYYLQNGRDMYVDQELDINRLSDYDVDHIVPQSFLKDDSIDNKV | |
| | LTRSDKNRGKSDNVPSEEVVKKMKNYWRQLLNAKLITQRKFDNLTKAERGGLSELDKAGFIKRQLVET | |
| | RQITKHVAQILDSRMNTKYDENDKLIREVKVITLKSKLVSDFRKDFQFYKVREINNYHHAHDAYLNAV | |
| | VGTALIKKYPKLESEFVYGDYKVYDVRKMIAKSEQSGGSKRTADGSEFEPKKKRKV | |
| CP1041-ABE8e | MKRTADGSEFESPKKKRKVSEVEFSHEYWMRHALTLAKRARDEREVPVGAVLVLNNRVIGEGWNRAIG | 218 |
| | LHDPTAHAEIMALRQGGLVMQNYRLIDATLYVTFEPCVMCAGAMIHSRIGRVVFGVRNSKRGAAGSLM | |
| | NVLNYPGMNHRVEITEGILADECAALLCDFYRMPRQVFNAQKKAQSSINSGGSSGGSSGSETPGTSES | |
| | ATPESSGGSSGGSNIMNFFKTEITLANGEIRKRPLIETNGETGEIVWDKGRDFATVRKVLSMPQVNIV | |
| | KKTEVQTGGFSKESILPKRNSDKLIARKKDWDPKKYGGFDSPTVAYSVLVVAKVEKGKSKKLKSVKEL | |
| | LGITIMERSSFEKNPIDFLEAKGYKEVKKDLIIKLPKYSLFELENGRKRMLASAGELQKGNELALPSK | |
| | YVNFLYLASHYEKLKGSPEDNEQKQLFVEQHKHYLDEIIEQISEFSKRVILADANLDKVLSAYNKHRD | |
| | KPIREQAENIIHLFTLTNLGAPAAFKYFDTTIDRKRYTSTKEVLDATLIHQSITGLYETRIDLSQLGG | |
| | DGGSGGSGGSGGSGGSGGSGGDKKYSIGLAIGTNSVGWAVITDEYKVPSKKFKVLGNTDRHSIKKNLI | |
| | GALLFDSGETAEATRLKRTARRRYTRRKNRICYLQEIFSNEMAKVDDSFFHRLEESFLVEEDKKHERH | |
| | PIFGNIVDEVAYHEKYPTIYHLRKKLVDSTDKADLRLIYLALAHMIKFRGHFLIEGDLNPDNSDVDKL | |
| | FIQLVQTYNQLFEENPINASGVDAKAILSARLSKSRRLENLIAQLPGEKKNGLFGNLIALSLGLTPNF | |
| | KSNFDLAEDAKLQLSKDTYDDDLDNLLAQIGDQYADLFLAAKNLSDAILLSDILRVNTEITKAPLSAS | |
| | MIKRYDEHHQDLTLLKALVRQQLPEKYKEIFFDQSKNGYAGYIDGGASQEEFYKFIKPILEKMDGTEE | |
| | LLVKLNREDLLRKQRTFDNGSIPHQIHLGELHAILRRQEDFYPFLKDNREKIEKILTFRIPYYVGPLA | |
| | RGNSRFAWMTRKSEETITPWNFEEVVDKGASAQSFIERMTNFDKNLPNEKVLPKHSLLYEYFTVYNEL | |
| | TKVKYVTEGMRKPAFLSGEQKKAIVDLLFKTNRKVTVKQLKEDYFKKIECFDSVEISGVEDRFNASLG | |
| | TYHDLLKIIKDKDFLDNEENEDILEDIVLTLTLFEDREMIEERLKTYAHLFDDKVMKQLKRRRYTGWG | |
| | RLSRKLINGIRDKQSGKTILDFLKSDGFANRNFMQLIHDDSLTFKEDIQKAQVSGQGDSLHEHIANLA | |
| | GSPAIKKGILQTVKVVDELVKVMGRHKPENIVIEMARENQTTQKGQKNSRERMKRIEEGIKELGSQIL | |
| | KEHPVENTQLQNEKLYLYYLQNGRDMYVDQELDINRLSDYDVDHIVPQSFLKDDSIDNKVLTRSDKNR | |
| | GKSDNVPSEEVVKKMKNYWRQLLNAKLITQRKFDNLTKAERGGLSELDKAGFIKRQLVETRQITKHVA | |
| | QILDSRMNTKYDENDKLIREVKVITLKSKLVSDFRKDFQFYKVREINNYHHAHDAYLNAVVGTALIKK | |
| | YPKLESEFVYGDYKVYDVRKMIAKSEQEIGKATAKYFFYSSGGSKRTADGSEFEPKKKRKV | |
| ABE8e (TadA-8e | MKRTADGSEFESPKKKRKVSEVEFSHEYWMRHALTLAKRARDEREVPVGAVLVLNNRVIGEGWNRAIG | 219 |
| V82G) | LHDPTAHAEIMALRQGGLVMQNYRLIDATLYVTFEPCVMCAGAMIHSRIGRVVFGVRNSKRGAAGSLM | |
| | NVLNYPGMNHRVEITEGILADECAALLCDFYRMPRQVFNAQKKAQSSINSGGSSGGSSGSETPGTSES | |
| | ATPESSGGSSGGSDKKYSIGLAIGTNSVGWAVITDEYKVPSKKFKVLGNTDRHSIKKNLIGALLFDSG | |
| | ETAEATRLKRTARRRYTRRKNRICYLQEIFSNEMAKVDDSFFHRLEESFLVEEDKKHERHPIFGNIVD | |
| | EVAYHEKYPTIYHLRKKLVDSTDKADLRLIYLALAHMIKFRGHFLIEGDLNPDNSDVDKLFIQLVQTY | |
| | NQLFEENPINASGVDAKAILSARLSKSRRLENLIAQLPGEKKNGLFGNLIALSLGLTPNFKSNFDLAE | |
| | DAKLQLSKDTYDDDLDNLLAQIGDQYADLFLAAKNLSDAILLSDILRVNTEITKAPLSASMIKRYDEH | |
| | HQDLTLLKALVRQQLPEKYKEIFFDQSKNGYAGYIDGGASQEEFYKFIKPILEKMDGTEELLVKLNRE | |
| | DLLRKQRTFDNGSIPHQIHLGELHAILRRQEDFYPFLKDNREKIEKILTFRIPYYVGPLARGNSRFAW | |
| | MTRKSEETITPWNFEEVVDKGASAQSFIERMTNFDKNLPNEKVLPKHSLLYEYFTVYNELTKVKYVTE | |
| | GMRKPAFLSGEQKKAIVDLLFKTNRKVTVKQLKEDYFKKIECFDSVEISGVEDRFNASLGTYHDLLKI | |
| | IKDKDFLDNEENEDILEDIVLTLTLFEDREMIEERLKTYAHLFDDKVMKQLKRRRYTGWGRLSRKLIN | |
| | GIRDKQSGKTILDFLKSDGFANRNFMQLIHDDSLTFKEDIQKAQVSGQGDSLHEHIANLAGSPAIKKG | |
| | ILQTVKVVDELVKVMGRHKPENIVIEMARENQTTQKGQKNSRERMKRIEEGIKELGSQILKEHPVENT | |
| | QLQNEKLYLYYLQNGRDMYVDQELDINRLSDYDVDHIVPQSFLKDDSIDNKVLTRSDKNRGKSDNVPS | |
| | EEVVKKMKNYWRQLLNAKLITQRKFDNLTKAERGGLSELDKAGFIKRQLVETRQITKHVAQILDSRMN | |
| | TKYDENDKLIREVKVITLKSKLVSDFRKDFQFYKVREINNYHHAHDAYLNAVVGTALIKKYPKLESEF | |
| | VYGDYKVYDVRKMIAKSEQEIGKATAKYFFYSNIMNFFKTEITLANGEIRKRPLIETNGETGEIVWDK | |
| | GRDFATVRKVLSMPQVNIVKKTEVQTGGFSKESILPKRNSDKLIARKKDWDPKKYGGFDSPTVAYSVL | |
| | VVAKVEKGKSKKLKSVKELLGITIMERSSFEKNPIDFLEAKGYKEVKKDLIIKLPKYSLFELENGRKR | |
| | MLASAGELQKGNELALPSKYVNFLYLASHYEKLKGSPEDNEQKQLFVEQHKHYLDEIIEQISEFSKRV | |
| | ILADANLDKVLSAYNKHRDKPIREQAENIIHLFTLTNLGAPAAFKYFDTTIDRKRYTSTKEVLDATLI | |
| | HQSITGLYETRIDLSQLGGDSGGSKRTADGSEFEPKKKRKV | |
| ABE8e (TadA-8e | MKRTADGSEFESPKKKRKVSEVEFSHEYWMRHALTLAKRARDEREVPVGAVLVLNNRVIGEGWNRAIG | 220 |
| K2 0AR2 1A) | LHDPTAHAEIMALRQGGLVMQNYRLIDATLYVTFEPCVMCAGAMIHSRIGRVVFGVRNSKRGAAGSLM | |
| | NVLNYPGMNHRVEITEGILADECAALLCDFYRMPRQVFNAQKKAQSSINSGGSSGGSSGSETPGTSES | |
| | ATPESSGGSSGGSDKKYSIGLAIGTNSVGWAVITDEYKVPSKKFKVLGNTDRHSIKKNLIGALLFDSG | |
| | ETAEATRLKRTARRRYTRRKNRICYLQEIFSNEMAKVDDSFFHRLEESFLVEEDKKHERHPIFGNIVD | |
| | EVAYHEKYPTIYHLRKKLVDSTDKADLRLIYLALAHMIKFRGHFLIEGDLNPDNSDVDKLFIQLVQTY | |
| | NQLFEENPINASGVDAKAILSARLSKSRRLENLIAQLPGEKKNGLFGNLIALSLGLTPNFKSNFDLAE | |
| | DAKLQLSKDTYDDDLDNLLAQIGDQYADLFLAAKNLSDAILLSDILRVNTEITKAPLSASMIKRYDEH | |
| | HQDLTLLKALVRQQLPEKYKEIFFDQSKNGYAGYIDGGASQEEFYKFIKPILEKMDGTEELLVKLNRE | |
| | DLLRKQRTFDNGSIPHQIHLGELHAILRRQEDFYPFLKDNREKIEKILTFRIPYYVGPLARGNSRFAW | |
| | MTRKSEETITPWNFEEVVDKGASAQSFIERMTNFDKNLPNEKVLPKHSLLYEYFTVYNELTKVKYVTE | |
| | GMRKPAFLSGEQKKAIVDLLFKTNRKVTVKQLKEDYFKKIECFDSVEISGVEDRFNASLGTYHDLLKI | |
| | IKDKDFLDNEENEDILEDIVLTLTLFEDREMIEERLKTYAHLFDDKVMKQLKRRRYTGWGRLSRKLIN | |
| | GIRDKQSGKTILDFLKSDGFANRNFMQLIHDDSLTFKEDIQKAQVSGQGDSLHEHIANLAGSPAIKKG | |
| | ILQTVKVVDELVKVMGRHKPENIVIEMARENQTTQKGQKNSRERMKRIEEGIKELGSQILKEHPVENT | |
| | QLQNEKLYLYYLQNGRDMYVDQELDINRLSDYDVDHIVPQSFLKDDSIDNKVLTRSDKNRGKSDNVPS | |
| | EEVVKKMKNYWRQLLNAKLITQRKFDNLTKAERGGLSELDKAGFIKRQLVETRQITKHVAQILDSRMN | |
| | TKYDENDKLIREVKVITLKSKLVSDFRKDFQFYKVREINNYHHAHDAYLNAVVGTALIKKYPKLESEF | |
| | VYGDYKVYDVRKMIAKSEQEIGKATAKYFFYSNIMNFFKTEITLANGEIRKRPLIETNGETGEIVWDK | |
| | GRDFATVRKVLSMPQVNIVKKTEVQTGGFSKESILPKRNSDKLIARKKDWDPKKYGGFDSPTVAYSVL | |
| | VVAKVEKGKSKKLKSVKELLGITIMERSSFEKNPIDFLEAKGYKEVKKDLIIKLPKYSLFELENGRKR | |
| | MLASAGELQKGNELALPSKYVNFLYLASHYEKLKGSPEDNEQKQLFVEQHKHYLDEIIEQISEFSKRV | |
| | ILADANLDKVLSAYNKHRDKPIREQAENIIHLFTLTNLGAPAAFKYFDTTIDRKRYTSTKEVLDATLI | |
| | HQSITGLYETRIDLSQLGGDSGGSKRTADGSEFEPKKKRKV | |
| ABE8-SpCas9 | MKRTADGSEFESPKKKRKVSEVEFSHEYWMRHALTLAKRAWDEREVPVGAVLVHNNRVIGEGWNRPIG | 221 |
| editor (AA) | RHDPTAHAEIMALRQGGLVMQNYRLIDATLYVTLEPCVMCAGAMIHSRIGRVVFGARDAKTGAAGSLM | |
| NLS, wtTadA, | DVLHHPGMNHRVEITEGILADECAALLSDFFRMRRQEIKAQKKAQSSTDSGGSSGGSSGSETPGTSES | |
| linker, TadA*, | ATPESSGGSSGGSGTTGATGGAGCTCTGGGCCTTCTTCTGAGCATTGAACACCTGTCTAGGCATCCGA | |
| Cas 9 | TAGAAATCGCACAGCAGGGCGGCACATTCATCTGCCAGGATTCCCTCGGTAATTTCGACGCGGTGATT | |
| | CATGCCGGGGTAGTTCAGCACGTTCATCAGGGAGCCTGCGGCGCCTCTTTTTGAGTTCCTCACGCCAA | |
| | ACACCACGCGGCCGATCCTAGAGTGGATCATGGCGCCGGCGCACATCACGCAAGGCTCGAATGTCACG | |
| | TACAGGGTGGCGTCAATCAGTCTGTAGTTCTGCATGACCAGGCCGCCCTGTCTCAGGGCCATAATTTC | |
| | GGCATGGGCTGTTGGGTCGTGCAGGCCGATGGCTCTGTTCCAGCCCTCGCCGATCACTCTATTGTTCA | |
| | GCACCAGCACGGCTCCCACAGGCACCTCCCTCTCATCCCGTGCCCTCTTGGCCAGGGTCAGGGCATGT | |
| | CTCATCCAGTACTCGTGGGAAAACTCCACCTCAGASGGSSGGSSGSETPGTSESATPESSGGSSGGSD | |
| | KKYSIGLAIGTNSVGWAVITDEYKVPSKKFKVLGNTDRHSIKKNLIGALLFDSGETAEATRLKRTARR | |
| | RYTRRKNRICYLQEIFSNEMAKVDDSFFHRLEESFLVEEDKKHERHPIFGNIVDEVAYHEKYPTIYHL | |
| | RKKLVDSTDKADLRLIYLALAHMIKFRGHFLIEGDLNPDNSDVDKLFIQLVQTYNQLFEENPINASGV | |
| | DAKAILSARLSKSRRLENLIAQLPGEKKNGLFGNLIALSLGLTPNFKSNFDLAEDAKLQLSKDTYDDD | |
| | LDNLLAQIGDQYADLFLAAKNLSDAILLSDILRVNTEITKAPLSASMIKRYDEHHQDLTLLKALVRQQ | |
| | LPEKYKEIFFDQSKNGYAGYIDGGASQEEFYKFIKPILEKMDGTEELLVKLNREDLLRKQRTFDNGSI | |
| | PHQIHLGELHAILRRQEDFYPFLKDNREKIEKILTFRIPYYVGPLARGNSRFAWMTRKSEETITPWNF | |
| | EEVVDKGASAQSFIERMTNFDKNLPNEKVLPKHSLLYEYFTVYNELTKVKYVTEGMRKPAFLSGEQKK | |
| | AIVDLLFKTNRKVTVKQLKEDYFKKIECFDSVEISGVEDRFNASLGTYHDLLKIIKDKDFLDNEENED | |
| | ILEDIVLTLTLFEDREMIEERLKTYAHLFDDKVMKQLKRRRYTGWGRLSRKLINGIRDKQSGKTILDF | |
| | LKSDGFANRNFMQLIHDDSLTFKEDIQKAQVSGQGDSLHEHIANLAGSPAIKKGILQTVKVVDELVKV | |
| | MGRHKPENIVIEMARENQTTQKGQKNSRERMKRIEEGIKELGSQILKEHPVENTQLQNEKLYLYYLQN | |
| | GRDMYVDQELDINRLSDYDVDHIVPQSFLKDDSIDNKVLTRSDKNRGKSDNVPSEEVVKKMKNYWRQL | |
| | LNAKLITQRKFDNLTKAERGGLSELDKAGFIKRQLVETRQITKHVAQILDSRMNTKYDENDKLIREVK | |
| | VITLKSKLVSDFRKDFQFYKVREINNYHHAHDAYLNAVVGTALIKKYPKLESEFVYGDYKVYDVRKMI | |
| | AKSEQEIGKATAKYFFYSNIMNFFKTEITLANGEIRKRPLIETNGETGEIVWDKGRDFATVRKVLSMP | |
| | QVNIVKKTEVQTGGFSKESILPKRNSDKLIARKKDWDPKKYGGFDSPTVAYSVLVVAKVEKGKSKKLK | |
| | SVKELLGITIMERSSFEKNPIDFLEAKGYKEVKKDLIIKLPKYSLFELENGRKRMLASAGELQKGNEL | |
| | ALPSKYVNFLYLASHYEKLKGSPEDNEQKQLFVEQHKHYLDEIIEQISEFSKRVILADANLDKVLSAY | |
| | NKHRDKPIREQAENIIHLFTLTNLGAPAAFKYFDTTIDRKRYTSTKEVLDATLIHQSITGLYETRIDL | |
| | SQLGGDSGGSKRTADGSEFEPKKKRKV | |
| ABE8-SpCas9 | ATGAAACGGACAGCCGACGGAAGCGAGTTCGAGTCACCAAAGAAGAAGCGGAAAGTCTCTGAAGTCGA | 222 |
| editor (NT) | GTTTAGCCACGAGTATTGGATGAGGCACGCACTGACCCTGGCAAAGCGAGCATGGGATGAAAGAGAAG | |
| NLS, WtTadA, | TCCCCGTGGGCGCCGTGCTGGTGCACAACAATAGAGTGATCGGAGAGGGATGGAACAGGCCAATCGGC | |
| linker, TadA*, | CGCCACGACCCTACCGCACACGCAGAGATCATGGCACTGAGGCAGGGAGGCCTGGTCATGCAGAATTA | |
| Cas 9 | CCGCCTGATCGATGCCACCCTGTATGTGACACTGGAGCCATGCGTGATGTGCGCAGGAGCAATGATCC | |
| | ACAGCAGGATCGGAAGAGTGGTGTTCGGAGCACGGGACGCCAAGACCGGCGCAGCAGGCTCCCTGATG | |
| | GATGTGCTGCACCACCCCGGCATGAACCACCGGGTGGAGATCACAGAGGGAATCCTGGCAGACGAGTG | |
| | CGCCGCCCTGCTGAGCGATTTCTTTAGAATGCGGAGACAGGAGATCAAGGCCCAGAAGAAGGCACAGA | |
| | GCTCCACCGACTCTGGAGGATCTAGCGGAGGATCCTCTGGAAGCGAGACACCAGGCACAAGCGAGTCC | |
| | GCCACACCAGAGAGCTCCGGCGGCTCCTCCGGAGGATCCTCTGAGGTGGAGTTTTCCCACGAGTACTG | |
| | GATGAGACATGCCCTGACCCTGGCCAAGAGGGCACGGGATGAGAGGGAGGTGCCTGTGGGAGCCGTGC | |
| | TGGTGCTGAACAATAGAGTGATCGGCGAGGGCTGGAACAGAGCCATCGGCCTGCACGACCCAACAGCC | |
| | CATGCCGAAATTATGGCCCTGAGACAGGGCGGCCTGGTCATGCAGAACTACAGACTGATTGACGCCAC | |
| | CCTGTACGTGACATTCGAGCCTTGCGTGATGTGCGCCGGCGCCATGATCCACTCTAGGATCGGCCGCG | |
| | TGGTGTTTGGCGTGAGGAACTCAAAAAGAGGCGCCGCAGGCTCCCTGATGAACGTGCTGAACTACCCC | |
| | GGCATGAATCACCGCGTCGAAATTACCGAGGGAATCCTGGCAGATGAATGTGCCGCCCTGCTGTGCGA | |
| | TTTCTATCGGATGCCTAGACAGGTGTTCAATGCTCAGAAGAAGGCCCAGAGCTCCATCAACAGTGGTG | |
| | GAAGTAGCGGAGGCTCCTCTGGCTCTGAGACACCTGGCACAAGCGAGAGCGCAACACCTGAAAGCAGC | |
| | GGGGGCAGCAGCGGGGGGTCAGACAAGAAGTACAGCATCGGCCTGGCCATCGGCACCAACTCTGTGGG | |
| | CTGGGCCGTGATCACCGACGAGTACAAGGTGCCCAGCAAGAAATTCAAGGTGCTGGGCAACACCGACC | |
| | GGCACAGCATCAAGAAGAACCTGATCGGAGCCCTGCTGTTCGACAGCGGCGAAACAGCCGAGGCCACC | |
| | CGGCTGAAGAGAACCGCCAGAAGAAGATACACCAGACGGAAGAACCGGATCTGCTATCTGCAAGAGAT | |
| | CTTCAGCAACGAGATGGCCAAGGTGGACGACAGCTTCTTCCACAGACTGGAAGAGTCCTTCCTGGTGG | |
| | AAGAGGATAAGAAGCACGAGCGGCACCCCATCTTCGGCAACATCGTGGACGAGGTGGCCTACCACGAG | |
| | AAGTACCCCACCATCTACCACCTGAGAAAGAAACTGGTGGACAGCACCGACAAGGCCGACCTGCGGCT | |
| | GATCTATCTGGCCCTGGCCCACATGATCAAGTTCCGGGGCCACTTCCTGATCGAGGGCGACCTGAACC | |
| | CCGACAACAGCGACGTGGACAAGCTGTTCATCCAGCTGGTGCAGACCTACAACCAGCTGTTCGAGGAA | |
| | AACCCCATCAACGCCAGCGGCGTGGACGCCAAGGCCATCCTGTCTGCCAGACTGAGCAAGAGCAGACG | |
| | GCTGGAAAATCTGATCGCCCAGCTGCCCGGCGAGAAGAAGAATGGCCTGTTCGGAAACCTGATTGCCC | |
| | TGAGCCTGGGCCTGACCCCCAACTTCAAGAGCAACTTCGACCTGGCCGAGGATGCCAAACTGCAGCTG | |
| | AGCAAGGACACCTACGACGACGACCTGGACAACCTGCTGGCCCAGATCGGCGACCAGTACGCCGACCT | |
| | GTTTCTGGCCGCCAAGAACCTGTCCGACGCCATCCTGCTGAGCGACATCCTGAGAGTGAACACCGAGA | |
| | TCACCAAGGCCCCCCTGAGCGCCTCTATGATCAAGAGATACGACGAGCACCACCAGGACCTGACCCTG | |
| | CTGAAAGCTCTCGTGCGGCAGCAGCTGCCTGAGAAGTACAAAGAGATTTTCTTCGACCAGAGCAAGAA | |
| | CGGCTACGCCGGCTACATTGACGGCGGAGCCAGCCAGGAAGAGTTCTACAAGTTCATCAAGCCCATCC | |
| | TGGAAAAGATGGACGGCACCGAGGAACTGCTCGTGAAGCTGAACAGAGAGGACCTGCTGCGGAAGCAG | |
| | CGGACCTTCGACAACGGCAGCATCCCCCACCAGATCCACCTGGGAGAGCTGCACGCCATTCTGCGGCG | |
| | GCAGGAAGATTTTTACCCATTCCTGAAGGACAACCGGGAAAAGATCGAGAAGATCCTGACCTTCCGCA | |
| | TCCCCTACTACGTGGGCCCTCTGGCCAGGGGAAACAGCAGATTCGCCTGGATGACCAGAAAGAGCGAG | |
| | GAAACCATCACCCCCTGGAACTTCGAGGAAGTGGTGGACAAGGGCGCTTCCGCCCAGAGCTTCATCGA | |
| | GCGGATGACCAACTTCGATAAGAACCTGCCCAACGAGAAGGTGCTGCCCAAGCACAGCCTGCTGTACG | |
| | AGTACTTCACCGTGTATAACGAGCTGACCAAAGTGAAATACGTGACCGAGGGAATGAGAAAGCCCGCC | |
| | TTCCTGAGCGGCGAGCAGAAAAAGGCCATCGTGGACCTGCTGTTCAAGACCAACCGGAAAGTGACCGT | |
| | GAAGCAGCTGAAAGAGGACTACTTCAAGAAAATCGAGGACAAGAAGTACAGCATCGGCCTGGCCATCG | |
| | GCACCAACTCTGTGGGCTGGGCCGTGATCACCGACGAGTACAAGGTGCCCAGCAAGAAATTCAAGGTG | |
| | CTGGGCAACACCGACCGGCACAGCATCAAGAAGAACCTGATCGGAGCCCTGCTGTTCGACAGCGGCGA | |
| | AACAGCCGAGGCCACCCGGCTGAAGAGAACCGCCAGAAGAAGATACACCAGACGGAAGAACCGGATCT | |
| | GCTATCTGCAAGAGATCTTCAGCAACGAGATGGCCAAGGTGGACGACAGCTTCTTCCACAGACTGGAA | |
| | GAGTCCTTCCTGGTGGAAGAGGATAAGAAGCACGAGCGGCACCCCATCTTCGGCAACATCGTGGACGA | |
| | GGTGGCCTACCACGAGAAGTACCCCACCATCTACCACCTGAGAAAGAAACTGGTGGACAGCACCGACA | |
| | AGGCCGACCTGCGGCTGATCTATCTGGCCCTGGCCCACATGATCAAGTTCCGGGGCCACTTCCTGATC | |
| | GAGGGCGACCTGAACCCCGACAACAGCGACGTGGACAAGCTGTTCATCCAGCTGGTGCAGACCTACAA | |
| | CCAGCTGTTCGAGGAAAACCCCATCAACGCCAGCGGCGTGGACGCCAAGGCCATCCTGTCTGCCAGAC | |
| | TGAGCAAGAGCAGACGGCTGGAAAATCTGATCGCCCAGCTGCCCGGCGAGAAGAAGAATGGCCTGTTC | |
| | GGAAACCTGATTGCCCTGAGCCTGGGCCTGACCCCCAACTTCAAGAGCAACTTCGACCTGGCCGAGGA | |
| | TGCCAAACTGCAGCTGAGCAAGGACACCTACGACGACGACCTGGACAACCTGCTGGCCCAGATCGGCG | |
| | ACCAGTACGCCGACCTGTTTCTGGCCGCCAAGAACCTGTCCGACGCCATCCTGCTGAGCGACATCCTG | |
| | AGAGTGAACACCGAGATCACCAAGGCCCCCCTGAGCGCCTCTATGATCAAGAGATACGACGAGCACCA | |
| | CCAGGACCTGACCCTGCTGAAAGCTCTCGTGCGGCAGCAGCTGCCTGAGAAGTACAAAGAGATTTTCT | |
| | TCGACCAGAGCAAGAACGGCTACGCCGGCTACATTGACGGCGGAGCCAGCCAGGAAGAGTTCTACAAG | |
| | TTCATCAAGCCCATCCTGGAAAAGATGGACGGCACCGAGGAACTGCTCGTGAAGCTGAACAGAGAGGA | |
| | CCTGCTGCGGAAGCAGCGGACCTTCGACAACGGCAGCATCCCCCACCAGATCCACCTGGGAGAGCTGC | |
| | ACGCCATTCTGCGGCGGCAGGAAGATTTTTACCCATTCCTGAAGGACAACCGGGAAAAGATCGAGAAG | |
| | ATCCTGACCTTCCGCATCCCCTACTACGTGGGCCCTCTGGCCAGGGGAAACAGCAGATTCGCCTGGAT | |
| | GACCAGAAAGAGCGAGGAAACCATCACCCCCTGGAACTTCGAGGAAGTGGTGGACAAGGGCGCTTCCG | |
| | CCCAGAGCTTCATCGAGCGGATGACCAACTTCGATAAGAACCTGCCCAACGAGAAGGTGCTGCCCAAG | |
| | CACAGCCTGCTGTACGAGTACTTCACCGTGTATAACGAGCTGACCAAAGTGAAATACGTGACCGAGGG | |
| | AATGAGAAAGCCCGCCTTCCTGAGCGGCGAGCAGAAAAAGGCCATCGTGGACCTGCTGTTCAAGACCA | |
| | ACCGGAAAGTGACCGTGAAGCAGCTGAAAGAGGACTACTTCAAGAAAATCGAGTCTGGCGGCTCAAAA | |
| | AGAACCGCCGACGGCAGCGAATTCGAGCCCAAGAAGAAGAGGAAAGTC | |
| ABE8-NRTH | NLS, wtTadA, linker, TadA*, NRCH | 463 |
| editor | Amino Acid Sequence | |
| | MKRTADGSEFESPKKKRKVSEVEFSHEYWMRHALTLAKRAWDEREVPVGAVLVHNNRVIGEGWNRPIG | |
| | RHDPTAHAEIMALRQGGLVMQNYRLIDATLYVTLEPCVMCAGAMIHSRIGRVVFGARDAKTGAAGSLM | |
| | DVLHHPGMNHRVEITEGILADECAALLSDFFRMRRQEIKAQKKAQSSTDSGGSSGGSSGSETPGTSES | |
| | ATPESSGGSSGGSGTTGATGGAGCTCTGGGCCTTCTTCTGAGCATTGAACACCTGTCTAGGCATCCGA | |
| | TAGAAATCGCACAGCAGGGCGGCACATTCATCTGCCAGGATTCCCTCGGTAATTTCGACGCGGTGATT | |
| | CATGCCGGGGTAGTTCAGCACGTTCATCAGGGAGCCTGCGGCGCCTCTTTTTGAGTTCCTCACGCCAA | |
| | ACACCACGCGGCCGATCCTAGAGTGGATCATGGCGCCGGCGCACATCACGCAAGGCTCGAATGTCACG | |
| | TACAGGGTGGCGTCAATCAGTCTGTAGTTCTGCATGACCAGGCCGCCCTGTCTCAGGGCCATAATTTC | |
| | GGCATGGGCTGTTGGGTCGTGCAGGCCGATGGCTCTGTTCCAGCCCTCGCCGATCACTCTATTGTTCA | |
| | GCACCAGCACGGCTCCCACAGGCACCTCCCTCTCATCCCGTGCCCTCTTGGCCAGGGTCAGGGCATGT | |
| | CTCATCCAGTACTCGTGGGAAAACTCCACCTCAGASGGSSGGSSGSETPGTSESATPESSGGSSGGSD | |
| | KKYSIGLAIGTNSVGWAVITDEYKVPSKKFKVLGNTDRHSIKKNLIGALLFDSGETAEATRLKRTARR | |
| | RYTRRKNRICYLQEIFSNEMAKVDDSFFHRLEESFLVEEDKKHERHPIFGNIVDEVAYHEKYPTIYHL | |
| | RKKLVDSTDKADLRLIYLALAHMIKFRGHFLIEGDLNPDNSDVDKLFIQLVQTYNQLFEENPINASGV | |
| | DAKAILSARLSKSRRLENLIAQLPGEKKNGLFGNLIALSLGLTPNFKSNFDLAEDAKLQLSKDTYDDD | |
| | LDNLLAQIGDQYADLFLAAKNLSDAILLSDILRVNTEITKAPLSASMVKRYDEHHQDLTLLKALVRQQ | |
| | LPEKYKEIFFDQSKNGYAGYIDGGASQEEFYKFIKPILEKMDGTEELLVKLNREDLLRKQRTFDNGII | |
| | PHQIHLGELHAILRRQGDFYPFLKDNREKIEKILTFRIPYYVGPLARGNSRFAWMTRKSEETITPWNF | |
| | EEVVDKGASAQSFIERMTNFDKNLPNEKVLPKHSLLYEYFTVYNELTKVKYVTEGMRKPAFLSGEQKK | |
| | AIVDLLFKTNRKVTVKQLKEDYFKKIECFDSVEISGVEDRFNASLGTYHDLLKIIKDKDFLDNEENED | |
| | ILEDIVLTLTLFEDREMIEERLKTYAHLFDDKVMKQLKRLRYTGWGRLSRKLINGIRDKQSGKTILDF | |
| | LKSDGFANRNFMQLIHDDSLTFKEDIQKAQVSCQGDSLHEHIANLAGSPAIKKGILQTVKVVDELIKV | |
| | MGGHKPENIVIEMARENQTTQKGQKNSRERMKRIEEGIKELGSQILKEHPVENTQLQNEKLYLYYLQN | |
| | GRDMYVDQELDINRLSDYDVDHIVPQSFLKDDSIENKVLTRSDKNRGKSDNVPSEEVVKKMKNYWRQL | |
| | LNAKLITQRKFDNLTKAERGGLSELDKAGFIKRQLAETRQITKHVAQILDSRMNTKYDENDKLIREVK | |
| | VITLKSKLVSDFRKDFQFYKVREINNYHHAHDAYLNAVVGTALIKKYPKLESEFVYGDYKVYDVRKMI | |
| | AKSEQEIGKATAKYFFYSNIMNFFKTEITLANGEIRKRPLIETNGETGEIVWDKGRDFATVRKVLSMP | |
| | QVNIVKKTEVQTGGFSKESILPKGNSDKLIARKKDWDPKKYGGFNSPTVAYSVLVVAKVEKGKSKKLK | |
| | SVKELLGITIMERSSFEKNPIGFLEAKGYKEVKKDLIIKLPKYSLFELENGRKRMLASASVLHKGNEL | |
| | ALPSKYVNFLYLASHYEKLKGSSEDNKQKQLFVEQHKHYLDEIIEQISEFSKRVILADANLDKVLSAY | |
| | NKHRDKPIREQAENIIHLFTLTNLGASAAFKYFDTTIGRKLYTSTKEVLDATLIHQSITGLYETRIDL | |
| | SQLGGDSGGSKRTADGSEFEPKKKRKV | |
| ABE-SpyMac | NLS, wtTadA, linker, TadA*, NRCH | 464 |
| editor | Amino Acid Sequence | |
| | MKRTADGSEFESPKKKRKVSEVEFSHEYWMRHALTLAKRAWDEREVPVGAVLVHNNRVIGEGWNRPIG | |
| | RHDPTAHAEIMALRQGGLVMQNYRLIDATLYVTLEPCVMCAGAMIHSRIGRVVFGARDAKTGAAGSLM | |
| | DVLHHPGMNHRVEITEGILADECAALLSDFFRMRRQEIKAQKKAQSSTDSGGSSGGSSGSETPGTSES | |
| | ATPESSGGSSGGSGTTGATGGAGCTCTGGGCCTTCTTCTGAGCATTGAACACCTGTCTAGGCATCCGA | |
| | TAGAAATCGCACAGCAGGGCGGCACATTCATCTGCCAGGATTCCCTCGGTAATTTCGACGCGGTGATT | |
| | CATGCCGGGGTAGTTCAGCACGTTCATCAGGGAGCCTGCGGCGCCTCTTTTTGAGTTCCTCACGCCAA | |
| | ACACCACGCGGCCGATCCTAGAGTGGATCATGGCGCCGGCGCACATCACGCAAGGCTCGAATGTCACG | |
| | TACAGGGTGGCGTCAATCAGTCTGTAGTTCTGCATGACCAGGCCGCCCTGTCTCAGGGCCATAATTTC | |
| | GGCATGGGCTGTTGGGTCGTGCAGGCCGATGGCTCTGTTCCAGCCCTCGCCGATCACTCTATTGTTCA | |
| | GCACCAGCACGGCTCCCACAGGCACCTCCCTCTCATCCCGTGCCCTCTTGGCCAGGGTCAGGGCATGT | |
| | CTCATCCAGTACTCGTGGGAAAACTCCACCTCAGASGGSSGGSSGSETPGTSESATPESSGGSSGGSD | |
| | KKYSIGLAIGTNSVGWAVITDEYKVPSKKFKVLGNTDRHSIKKNLIGALLFDSGETAEATRLKRTARR | |
| | RYTRRKNRICYLQEIFSNEMAKVDDSFFHRLEESFLVEEDKKHERHPIFGNIVDEVAYHEKYPTIYHL | |
| | RKKLVDSTDKADLRLIYLALAHMIKFRGHFLIEGDLNPDNSDVDKLFIQLVQTYNQLFEENPINASGV | |
| | DAKAILSARLSKSRRLENLIAQLPGEKKNGLFGNLIALSLGLTPNFKSNFDLAEDAKLQLSKDTYDDD | |
| | LDNLLAQIGDQYADLFLAAKNLSDAILLSDILRVNTEITKAPLSASMIKRYDEHHQDLTLLKALVRQQ | |
| | LPEKYKEIFFDQSKNGYAGYIDGGASQEEFYKFIKPILEKMDGTEELLVKLNREDLLRKQRTFDNGSI | |
| | PHQIHLGELHAILRRQEDFYPFLKDNREKIEKILTFRIPYYVGPLARGNSRFAWMTRKSEETITPWNF | |
| | EEVVDKGASAQSFIERMTNFDKNLPNEKVLPKHSLLYEYFTVYNELTKVKYVTEGMRKPAFLSGEQKK | |
| | AIVDLLFKTNRKVTVKQLKEDYFKKIECFDSVEISGVEDRFNASLGTYHDLLKIIKDKDFLDNEENED | |
| | ILEDIVLTLTLFEDREMIEERLKTYAHLFDDKVMKQLKRRRYTGWGRLSRKLINGIRDKQSGKTILDF | |
| | LKSDGFANRNFMQLIHDDSLTFKEDIQKAQVSGQGDSLHEHIANLAGSPAIKKGILQTVKVVDELVKV | |
| | MGRHKPENIVIEMARENQTTQKGQKNSRERMKRIEEGIKELGSQILKEHPVENTQLQNEKLYLYYLQN | |
| | GRDMYVDQELDINRLSDYDVDHIVPQSFLKDDSIDNKVLTRSDKNRGKSDNVPSEEVVKKMKNYWRQL | |
| | LNAKLITQRKFDNLTKAERGGLSELDKAGFIKRQLVETRQITKHVAQILDSRMNTKYDENDKLIREVK | |
| | VITLKSKLVSDFRKDFQFYKVREINNYHHAHDAYLNAVVGTALIKKYPKLESEFVYGDYKVYDVRKMI | |
| | AKSEQEIGKATAKYFFYSNIMNFFKTEITLANGEIRKRPLIETNGETGEIVWDKGRDFATVRKVLSMP | |
| | QVNIVKKTEIQTVGQNGGLFDDNPKSPLEVTPSKLVPLKKELNPKKYGGYQKPTTAYPVLLITDTKQL | |
| | IPISVMNKKQFEQNPVKFLRDRGYQQVGKNDFIKLPKYTLVDIGDGIKRLWASSKEIHKGNQLVVSKK | |
| | SQILLYHAHHLDSDLSNDYLQNHNQQFDVLFNEIISFSKKCKLGKEHIQKIENVYSNKKNSASIEELA | |
| | ESFIKLLGFTQLGATSPFNFLGVKLNQKQYKGKKDYILPCTEGTLIRQSITGLYETRVDLSKIGEDSG | |
| | GSKRTADGSEFEPKKKRKV | |
| ABE8-VRQR- | NLS, wtTadA, linker, TadA*, NRCH | 465 |
| CP1041 editor | Amino Acid Sequence | |
| | MKRTADGSEFESPKKKRKVSEVEFSHEYWMRHALTLAKRAWDEREVPVGAVLVHNNRVIGEGWNRPIG | |
| | RHDPTAHAEIMALRQGGLVMQNYRLIDATLYVTLEPCVMCAGAMIHSRIGRVVFGARDAKTGAAGSLM | |
| | DVLHHPGMNHRVEITEGILADECAALLSDFFRMRRQEIKAQKKAQSSTDSGGSSGGSSGSETPGTSES | |
| | ATPESSGGSSGGSGTTGATGGAGCTCTGGGCCTTCTTCTGAGCATTGAACACCTGTCTAGGCATCCGA | |
| | TAGAAATCGCACAGCAGGGCGGCACATTCATCTGCCAGGATTCCCTCGGTAATTTCGACGCGGTGATT | |
| | CATGCCGGGGTAGTTCAGCACGTTCATCAGGGAGCCTGCGGCGCCTCTTTTTGAGTTCCTCACGCCAA | |
| | ACACCACGCGGCCGATCCTAGAGTGGATCATGGCGCCGGCGCACATCACGCAAGGCTCGAATGTCACG | |
| | TACAGGGTGGCGTCAATCAGTCTGTAGTTCTGCATGACCAGGCCGCCCTGTCTCAGGGCCATAATTTC | |
| | GGCATGGGCTGTTGGGTCGTGCAGGCCGATGGCTCTGTTCCAGCCCTCGCCGATCACTCTATTGTTCA | |
| | GCACCAGCACGGCTCCCACAGGCACCTCCCTCTCATCCCGTGCCCTCTTGGCCAGGGTCAGGGCATGT | |
| | CTCATCCAGTACTCGTGGGAAAACTCCACCTCAGASGGSSGGSSGSETPGTSESATPESSGGSSGGSN | |
| | IMNFFKTEITLANGEIRKRPLIETNGETGEIVWDKGRDFATVRKVLSMPQVNIVKKTEVQTGGFSKES | |
| | IRPKRNSDKLIARKKDWDPKKYGGFVSPTVAYSVLVVAKVEKGKSKKLKSVKELLGITIMERSSFEKN | |
| | PIDFLEAKGYKEVKKDLIIKLPKYSLFELENGRKRMLASARFLQKGNELALPSKYVNFLYLASHYEKL | |
| | KGSPEDNEQKQLFVEQHKHYLDEIIEQISEFSKRVILADANLDKVLSAYNKHRDKPIREQAENIIHLF | |
| | TLTNLGAPRAFKYFDTTIDRKVYRSTKEVLDATLIHQSITGLYETRIDLSQLGGDGGSGGSGGSGGSG | |
| | GSGGSGGDKKYSIGLAIGTNSVGWAVITDEYKVPSKKFKVLGNTDRHSIKKNLIGALLFDSGETAEAT | |
| | RLKRTARRRYTRRKNRICYLQEIFSNEMAKVDDSFFHRLEESFLVEEDKKHERHPIFGNIVDEVAYHE | |
| | KYPTIYHLRKKLVDSTDKADLRLIYLALAHMIKFRGHFLIEGDLNPDNSDVDKLFIQLVQTYNQLFEE | |
| | NPINASGVDAKAILSARLSKSRRLENLIAQLPGEKKNGLFGNLIALSLGLTPNFKSNFDLAEDAKLQL | |
| | SKDTYDDDLDNLLAQIGDQYADLFLAAKNLSDAILLSDILRVNTEITKAPLSASMIKRYDEHHQDLTL | |
| | LKALVRQQLPEKYKEIFFDQSKNGYAGYIDGGASQEEFYKFIKPILEKMDGTEELLVKLNREDLLRKQ | |
| | RTFDNGSIPHQIHLGELHAILRRQEDFYPFLKDNREKIEKILTFRIPYYVGPLARGNSRFAWMTRKSE | |
| | ETITPWNFEEVVDKGASAQSFIERMTNFDKNLPNEKVLPKHSLLYEYFTVYNELTKVKYVTEGMRKPA | |
| | FLSGEQKKAIVDLLFKTNRKVTVKQLKEDYFKKIECFDSVEISGVEDRFNASLGTYHDLLKIIKDKDF | |
| | LDNEENEDILEDIVLTLTLFEDREMIEERLKTYAHLFDDKVMKQLKRRRYTGWGRLSRKLINGIRDKQ | |
| | SGKTILDFLKSDGFANRNFMQLIHDDSLTFKEDIQKAQVSGQGDSLHEHIANLAGSPAIKKGILQTVK | |
| | VVDELVKVMGRHKPENIVIEMARENQTTQKGQKNSRERMKRIEEGIKELGSQILKEHPVENTQLQNEK | |
| | LYLYYLQNGRDMYVDQELDINRLSDYDVDHIVPQSFLKDDSIDNKVLTRSDKNRGKSDNVPSEEVVKK | |
| | MKNYWRQLLNAKLITQRKFDNLTKAERGGLSELDKAGFIKRQLVETRQITKHVAQILDSRMNTKYDEN | |
| | DKLIREVKVITLKSKLVSDFRKDFQFYKVREINNYHHAHDAYLNAWGTALIKKYPKLESEFVYGDYK | |
| | VYDVRKMIAKSEQEIGKATAKYFFYSSGGSKRTADGSEFEPKKKRKV | |
| ABE8-SaCas9 | NLS, wtTadA, linker, TadA*, NRCH | 466 |
| editor | Amino Acid Sequence | |
| | MKRTADGSEFESPKKKRKVSEVEFSHEYWMRHALTLAKRAWDEREVPVGAVLVHNNRVIGEGWNRPIG | |
| | RHDPTAHAEIMALRQGGLVMQNYRLIDATLYVTLEPCVMCAGAMIHSRIGRVVFGARDAKTGAAGSLM | |
| | DVLHHPGMNHRVEITEGILADECAALLSDFFRMRRQEIKAQKKAQSSTDSGGSSGGSSGSETPGTSES | |
| | ATPESSGGSSGGSGTTGATGGAGCTCTGGGCCTTCTTCTGAGCATTGAACACCTGTCTAGGCATCCGA | |
| | TAGAAATCGCACAGCAGGGCGGCACATTCATCTGCCAGGATTCCCTCGGTAATTTCGACGCGGTGATT | |
| | CATGCCGGGGTAGTTCAGCACGTTCATCAGGGAGCCTGCGGCGCCTCTTTTTGAGTTCCTCACGCCAA | |
| | ACACCACGCGGCCGATCCTAGAGTGGATCATGGCGCCGGCGCACATCACGCAAGGCTCGAATGTCACG | |
| | TACAGGGTGGCGTCAATCAGTCTGTAGTTCTGCATGACCAGGCCGCCCTGTCTCAGGGCCATAATTTC | |
| | GGCATGGGCTGTTGGGTCGTGCAGGCCGATGGCTCTGTTCCAGCCCTCGCCGATCACTCTATTGTTCA | |
| | GCACCAGCACGGCTCCCACAGGCACCTCCCTCTCATCCCGTGCCCTCTTGGCCAGGGTCAGGGCATGT | |
| | CTCATCCAGTACTCGTGGGAAAACTCCACCTCAGASGGSSGGSSGSETPGTSESATPESSGGSSGGSG | |
| | KRNYILGLAIGITSVGYGIIDYETRDVIDAGVRLFKEANVENNEGRRSKRGARRLKRRRRHRIQRVKK | |
| | LLFDYNLLTDHSELSGINPYEARVKGLSQKLSEEEFSAALLHLAKRRGVHNVNEVEEDTGNELSTKEQ | |
| | ISRNSKALEEKYVAELQLERLKKDGEVRGSINRFKTSDYVKEAKQLLKVQKAYHQLDQSFIDTYIDLL | |
| | ETRRTYYEGPGEGSPFGWKDIKEWYEMLMGHCTYFPEELRSVKYAYNADLYNALNDLNNLVITRDENE | |
| | KLEYYEKFQIIENVFKQKKKPTLKQIAKEILVNEEDIKGYRVTSTGKPEFTNLKVYHDIKDITARKEI | |
| | IENAELLDQIAKILTIYQSSEDIQEELTNLNSELTQEEIEQISNLKGYTGTHNLSLKAINLILDELWH | |
| | TNDNQIAIFNRLKLVPKKVDLSQQKEIPTTLVDDFILSPVVKRSFIQSIKVINAIIKKYGLPNDIIIE | |
| | LAREKNSKDAQKMINEMQKRNRQTNERIEEIIRTTGKENAKYLIEKIKLHDMQEGKCLYSLEAIPLED | |
| | LLNNPFNYEVDHIIPRSVSFDNSFNNKVLVKQEENSKKGNRTPFQYLSSSDSKISYETFKKHILNLAK | |
| | GKGRISKTKKEYLLEERDINRFSVQKDFINRNLVDTRYATRGLMNLLRSYFRVNNLDVKVKSINGGFT | |
| | SFLRRKWKFKKERNKGYKHHAEDALIIANADFIFKEWKKLDKAKKVMENQMFEEKQAESMPEIETEQE | |
| | YKEIFITPHQIKHIKDFKDYKYSHRVDKKPNRELINDTLYSTRKDDKGNTLIVNNLNGLYDKDNDKLK | |
| | KLINKSPEKLLMYHHDPQTYQKLKLIMEQYGDEKNPLYKYYEETGNYLTKYSKKDNGPVIKKIKYYGN | |
| | KLNAHLDITDDYPNSRNKVVKLSLKPYRFDVYLDNGVYKFVTVKNLDVIKKENYYEVNSKCYEEAKKL | |
| | KKISNQAEFIASFYNNDLIKINGELYRVIGVNNDLLNRIEVNMIDITYREYLENMNDKRPPRIIKTIA | |
| | SKTQSIKKYSTDILGNLYEVKSKKHPQIIKKGSGGSKRTADGSEFEPKKKRKV | |
| ABE8-NRCH | NLS, wtTadA, linker, TadA*, NRCH | 467 |
| editor | Amino Acid Sequence | |
| | MKRTADGSEFESPKKKRKVSEVEFSHEYWMRHALTLAKRAWDEREVPVGAVLVHNNRVIGEGWNRPIG | |
| | RHDPTAHAEIMALRQGGLVMQNYRLIDATLYVTLEPCVMCAGAMIHSRIGRVVFGARDAKTGAAGSLM | |
| | DVLHHPGMNHRVEITEGILADECAALLSDFFRMRRQEIKAQKKAQSSTDSGGSSGGSSGSETPGTSES | |
| | AYPESSGGSSGGSGTTGATGGAGCTCTGGGCCTTCTTCTGAGCATTGAACACCTGTCTAGGCATCCGA | |
| | TAGAAATCGCACAGCAGGGCGGCACATTCATCTGCCAGGATTCCCTCGGTAATTTCGACGCGGTGATT | |
| | CATGCCGGGGTAGTTCAGCACGTTCATCAGGGAGCCTGCGGCGCCTCTTTTTGAGTTCCTCACGCCAA | |
| | ACACCACGCGGCCGATCCTAGAGTGGATCATGGCGCCGGCGCACATCACGCAAGGCTCGAATGTCACG | |
| | TACAGGGTGGCGTCAATCAGTCTGTAGTTCTGCATGACCAGGCCGCCCTGTCTCAGGGCCATAATTTC | |
| | GGCATGGGCTGTTGGGTCGTGCAGGCCGATGGCTCTGTTCCAGCCCTCGCCGATCACTCTATTGTTCA | |
| | GCACCAGCACGGCTCCCACAGGCACCTCCCTCTCATCCCGTGCCCTCTTGGCCAGGGTCAGGGCATGT | |
| | CTCATCCAGTACTCGTGGGAAAACTCCACCTCAGASGGSSGGSSGSEYPGYSESAYPESSGGSSGGSD | |
| | KKYSIGLAIGTNSVGWAVITDEYKVPSKKFKVLGNTDRHSIKKNLIGALLFDSGETAEATRLKRTARR | |
| | RYTRRKNRICYLQEIFSNEMAKVDDSFFHRLEESFLVEEDKKHERHPIFGNIVDEVAYHEKYPTIYHL | |
| | RKKLVDSTDKADLRLIYLALAHMIKFRGHFLIEGDLNPDNSDVDKLFIQLVQTYNQLFEENPINASGV | |
| | DAKAILSARLSKSRRLENLIAQLPGEKKNGLFGNLIALSLGLTPNFKSNFDLAEDAKLQLSKDTYDDD | |
| | LDNLLAQIGDQYADLFLAAKNLSDAILLSDILRVNTEITKAPLSASMVKRYDEHHQDLTLLKALVRQQ | |
| | LPEKYKEIFFDQSKNGYAGYIDGGASQEEFYKFIKPILEKMDGTEELLVKLNREDLLRKQRTFDNGII | |
| | PHQIHLGELHAILRRQGDFYPFLKDNREKIEKILTFRIPYYVGPLARGNSRFAWMTRKSEETITPWNF | |
| | EEVVDKGASAQSFIERMTNFDKNLPNEKVLPKHSLLYEYFTVYNELTKVKYVTEGMRKPAFLSGEQKK | |
| | AIVDLLFKTNRKVTVKQLKEDYFKKIECFDSVEISGVEDRFNASLGTYHDLLKIIKDKDFLDNEENED | |
| | ILEDIVLTLTLFEDREMIEERLKTYAHLFDDKVMKQLKRLRYTGWGRLSRKLINGIRDKQSGKTILDF | |
| | LKSDGFANRNFMQLIHDDSLTFKEDIQKAQVSCQGDSLHEHIANLAGSPAIKKGILQTVKVVDELIKV | |
| | MGGHKPENIVIEMARENQTTQKGQKNSRERMKRIEEGIKELGSQILKEHPVENTQLQNEKLYLYYLQN | |
| | GRDMYVDQELDINRLSDYDVDHIVPQSFLKDDSIENKVLTRSDKNRGKSDNVPSEEVVKKMKNYWRQL | |
| | LNAKLITQRKFDNLTKAERGGLSELDKAGFIKRQLAETRQITKHVAQILDSRMNTKYDENDKLIREVK | |
| | VITLKSKLVSDFRKDFQFYKVREINNYHHAHDAYLNAVVGTALIKKYPKLESEFVYGDYKVYDVRKMI | |
| | AKSEQEIGKATAKYFFYSNIMNFFKTEITLANGEIRKRPLIETNGETGEIVWDKGRDFATVRKVLSMP | |
| | QVNIVKKTEVQTGGFSKESILPKGNSDKLIARKKDWDPKKYGGFNSPTVAYSVLVVAKVEKGKSKKLK | |
| | SVKELLGITIMERSSFEKNPIDFLEAKGYKEVKKDLIIKLPKYSLFELENGRKRMLASAGVLQKGNEL | |
| | ALPSKYVNFLYLASHYEKLKGSPEDNEQKQLFVEQHKHYLDEIIEQISEFSKRVILADANLDKVLSAY | |
| | NKHRDKPIREQAENIIHLFTLTNLGAPAAFKYFDTTINRKQYNTTKEVLDATLIRQSITGLYETRIDL | |
| | SQLGGDSGGSKRTADGSEFEPKKKRKV | |
| ABE8-NRRH | NLS, wtTadA, linker, TadA*, NRCH | 468 |
| editor | Amino Acid Sequence | |
| | MKRTADGSEFESPKKKRKVSEVEFSHEYWMRHALTLAKRAWDEREVPVGAVLVHNNRVIGEGWNRPIG | |
| | RHDPTAHAEIMALRQGGLVMQNYRLIDATLYVTLEPCVMCAGAMIHSRIGRVVFGARDAKTGAAGSLM | |
| | DVLHHPGMNHRVEITEGILADECAALLSDFFRMRRQEIKAQKKAQSSTDSGGSSGGSSGSETPGTSES | |
| | ATPESSGGSSGGSGTTGATGGAGCTCTGGGCCTTCTTCTGAGCATTGAACACCTGTCTAGGCATCCGA | |
| | TAGAAATCGCACAGCAGGGCGGCACATTCATCTGCCAGGATTCCCTCGGTAATTTCGACGCGGTGATT | |
| | CATGCCGGGGTAGTTCAGCACGTTCATCAGGGAGCCTGCGGCGCCTCTTTTTGAGTTCCTCACGCCAA | |
| | ACACCACGCGGCCGATCCTAGAGTGGATCATGGCGCCGGCGCACATCACGCAAGGCTCGAATGTCACG | |
| | TACAGGGTGGCGTCAATCAGTCTGTAGTTCTGCATGACCAGGCCGCCCTGTCTCAGGGCCATAATTTC | |
| | GGCATGGGCTGTTGGGTCGTGCAGGCCGATGGCTCTGTTCCAGCCCTCGCCGATCACTCTATTGTTCA | |
| | GCACCAGCACGGCTCCCACAGGCACCTCCCTCTCATCCCGTGCCCTCTTGGCCAGGGTCAGGGCATGT | |
| | CTCATCCAGTACTCGTGGGAAAACTCCACCTCAGASGGSSGGSSGSETPGTSESATPESSGGSSGGSD | |
| | KKYSIGLAIGTNSVGWAVITDEYKVPSKKFKVLGNTDRHSIKKNLIGALLFDSGETAEATRLKRTARR | |
| | RYTRRKNRICYLQEIFSNEMAKVDDSFFHRLEESFLVEEDKKHERHPIFGNIVDEVAYHEKYPTIYHL | |
| | RKKLVDSTDKADLRLIYLALAHMIKFRGHFLIEGDLNPDNSDVDKLFIQLVQTYNQLFEENPINASGV | |
| | DAKAILSARLSKSRRLENLIAQLPGEKKNGLFGNLIALSLGLTPNFKSNFDLAEDAKLQLSKDTYDDD | |
| | LDNLLAQIGDQYADLFLAAKNLSDAILLSDILRVNTEITKAPLSASMVKRYDEHHQDLTLLKALVRQQ | |
| | LPEKYKEIFFDQSKNGYAGYIDGGASQEEFYKFIKPILEKMDGTEELLVKLNREDLLRKQRTFDNGII | |
| | PHQIHLGELHAILRRQGDFYPFLKDNREKIEKILTFRIPYYVGPLARGNSRFAWMTRKSEETITPWNF | |
| | EEVVDKGASAQSFIERMTNFDKNLPNEKVLPKHSLLYEYFTVYNELTKVKYVTEGMRKPAFLSGEQKK | |
| | AIVDLLFKTNRKVTVKQLKEDYFKKIECFDSVEISGVEDRFNASLGTYHDLLKIIKDKDFLDNEENED | |
| | ILEDIVLTLTLFEDREMIEERLKTYAHLFDDKVMKQLKRLRYTGWGRLSRKLINGIRDKQSGKTILDF | |
| | LKSDGFANRNFMQLIHDDSLTFKEDIQKAQVSCQGDSLHEHIANLAGSPAIKKGILQTVKVVDELIKV | |
| | MGGHKPENIVIEMARENQTTQKGQKNSRERMKRIEEGIKELGSQILKEHPVENTQLQNEKLYLYYLQN | |
| | GRDMYVDQELDINRLSDYDVDHIVPQSFLKDDSIENKVLTRSDKNRGKSDNVPSEEVVKKMKNYWRQL | |
| | LNAKLITQRKFDNLTKAERGGLSELDKAGFIKRQLAETRQITKHVAQILDSRMNTKYDENDKLIREVK | |
| | VITLKSKLVSDFRKDFQFYKVREINNYHHAHDAYLNAVVGTALIKKYPKLESEFVYGDYKVYDVRKMI | |
| | AKSEQEIGKATAKYFFYSNIMNFFKTEITLANGEIRKRPLIETNGETGEIVWDKGRDFATVRKVLSMP | |
| | QVNIVKKTEVQTGGFSKESILPKGNSDKLIARKKDWDPKKYGGFNSPTAAYSVLVVAKVEKGKSKKLK | |
| | SVKELLGITIMERSSFEKNPIGFLEAKGYKEVKKDLIIKLPKYSLFELENGRKRMLASAGVLHKGNEL | |
| | ALPSKYVNFLYLASHYEKLKGSPEDNEQKQLFVEQHKHYLDEIIEQISEFSKRVILADANLDKVLSAY | |
| | NKHRDKPIREQAENIIHLFTLTNLGVPAAFKYFDTTIDKKRYTSTKEVLDATLIHQSITGLYETRIDL | |
| | SQLGGDSGGSKRTADGSEFEPKKKRKV | |
| ABE8-SaKKH | NLS, wtTadA, linker, TadA*, NRCH | 469 |
| editor | Amino Acid Sequence | |
| | MKRTADGSEFESPKKKRKVSEVEFSHEYWMRHALTLAKRAWDEREVPVGAVLVHNNRVIGEGWNRPIG | |
| | RHDPTAHAEIMALRQGGLVMQNYRLIDATLYVTLEPCVMCAGAMIHSRIGRVVFGARDAKTGAAGSLM | |
| | DVLHHPGMNHRVEITEGILADECAALLSDFFRMRRQEIKAQKKAQSSTDSGGSSGGSSGSETPGTSES | |
| | ATPESSGGSSGGSGTTGATGGAGCTCTGGGCCTTCTTCTGAGCATTGAACACCTGTCTAGGCATCCGA | |
| | TAGAAATCGCACAGCAGGGCGGCACATTCATCTGCCAGGATTCCCTCGGTAATTTCGACGCGGTGATT | |
| | CATGCCGGGGTAGTTCAGCACGTTCATCAGGGAGCCTGCGGCGCCTCTTTTTGAGTTCCTCACGCCAA | |
| | ACACCACGCGGCCGATCCTAGAGTGGATCATGGCGCCGGCGCACATCACGCAAGGCTCGAATGTCACG | |
| | TACAGGGTGGCGTCAATCAGTCTGTAGTTCTGCATGACCAGGCCGCCCTGTCTCAGGGCCATAATTTC | |
| | GGCATGGGCTGTTGGGTCGTGCAGGCCGATGGCTCTGTTCCAGCCCTCGCCGATCACTCTATTGTTCA | |
| | GCACCAGCACGGCTCCCACAGGCACCTCCCTCTCATCCCGTGCCCTCTTGGCCAGGGTCAGGGCATGT | |
| | CTCATCCAGTACTCGTGGGAAAACTCCACCTCAGASGGSSGGSSGSETPGTSESATPESSGGSSGGSG | |
| | GGAAGCGAAATTACATTCTGGGGCTGGCCATTGGCATTACATCAGTGGGCTATGGCATCATTGACTAC | |
| | GAGACAAGGGACGTGATCGACGCCGGCGTGAGACTGTTCAAGGAGGCCAACGTGGAGAACAATGAGGG | |
| | CCGGAGATCCAAGAGGGGAGCAAGGCGCCTGAAGCGGAGAAGGCGCCACAGAATCCAGAGAGTGAAGA | |
| | AGCTGCTGTTCGATTACAACCTGCTGACCGACCACTCCGAGCTGTCTGGCATCAATCCTTATGAGGCC | |
| | AGAGTGAAGGGCCTGTCCCAGAAGCTGTCTGAGGAGGAGTTTAGCGCCGCCCTGCTGCACCTGGCAAA | |
| | GAGGAGAGGCGTGCACAACGTGAATGAGGTGGAGGAGGACACCGGCAACGAGCTGTCCACAAAGGAGC | |
| | AGATCAGCCGCAATTCCAAGGCCCTGGAGGAGAAGTATGTGGCCGAGCTGCAGCTGGAGCGGCTGAAG | |
| | AAGGATGGCGAGGTGAGGGGCTCCATCAATCGCTTCAAGACCTCTGACTACGTGAAGGAGGCCAAGCA | |
| | GCTGCTGAAGGTGCAGAAGGCCTACCACCAGCTGGATCAGTCCTTTATCGATACATATATCGACCTGC | |
| | TGGAGACAAGGCGCACATACTATGAGGGACCAGGAGAGGGCTCTCCCTTCGGCTGGAAGGACATCAAG | |
| | GAGTGGTACGAGATGCTGATGGGCCACTGCACCTATTTTCCAGAGGAGCTGAGAAGCGTGAAGTACGC | |
| | CTATAACGCCGATCTGTACAACGCCCTGAATGACCTGAACAACCTGGTCATCACCAGGGATGAGAACG | |
| | AGAAGCTGGAGTACTATGAGAAGTTCCAGATCATCGAGAACGTGTTCAAGCAGAAGAAGAAGCCTACA | |
| | CTGAAGCAGATCGCCAAGGAGATCCTGGTGAACGAGGAGGACATCAAGGGCTACCGCGTGACCTCCAC | |
| | AGGCAAGCCAGAGTTCACCAATCTGAAGGTGTATCACGATATCAAGGACATCACAGCCCGGAAGGAGA | |
| | TCATCGAGAACGCCGAGCTGCTGGATCAGATCGCCAAGATCCTGACCATCTATCAGAGCTCCGAGGAC | |
| | ATCCAGGAGGAGCTGACCAACCTGAATAGCGAGCTGACACAGGAGGAGATCGAGCAGATCAGCAATCT | |
| | GAAGGGCTACACCGGCACACACAACCTGAGCCTGAAGGCCATCAATCTGATCCTGGATGAGCTGTGGC | |
| | ACACAAACGACAATCAGATCGCCATCTTTAACCGGCTGAAGCTGGTGCCAAAGAAGGTGGACCTGTCC | |
| | CAGCAGAAGGAGATCCCAACCACACTGGTGGACGATTTCATCCTGTCTCCCGTGGTGAAGCGGAGCTT | |
| | CATCCAGAGCATCAAAGTGATCAACGCCATCATCAAGAAGTACGGCCTGCCCAATGATATCATCATCG | |
| | AGCTGGCCAGGGAGAAGAACTCCAAGGACGCCCAGAAGATGATCAATGAGATGCAGAAGAGGAACCGC | |
| | CAGACCAATGAGCGGATCGAGGAGATCATCAGAACCACAGGCAAGGAGAACGCCAAGTACCTGATCGA | |
| | GAAGATCAAGCTGCACGATATGCAGGAGGGCAAGTGTCTGTATTCTCTGGAGGCCATCCCTCTGGAGG | |
| | ACCTGCTGAACAATCCATTCAACTACGAGGTGGATCACATCATCCCCCGGAGCGTGAGCTTCGACAAT | |
| | TCTTTTAACAATAAGGTGCTGGTGAAGCAGGAGGAGAACAGCAAGAAGGGCAATAGGACCCCTTTCCA | |
| | GTACCTGTCTAGCTCCGATTCTAAGATCAGCTACGAGACATTCAAGAAGCACATCCTGAATCTGGCCA | |
| | AGGGCAAGGGCCGCATCAGCAAGACCAAGAAGGAGTACCTGCTGGAGGAGCGGGACATCAACAGATTC | |
| | TCCGTGCAGAAGGACTTCATCAACCGGAATCTGGTGGACACCAGATACGCCACACGCGGCCTGATGAA | |
| | TCTGCTGCGGTCTTATTTCAGAGTGAACAATCTGGATGTGAAGGTGAAGAGCATCAACGGCGGCTTCA | |
| | CCTCCTTTCTGCGGAGAAAGTGGAAGTTTAAGAAGGAGCGCAACAAGGGCTATAAGCACCACGCCGAG | |
| | GATGCCCTGATCATCGCCAATGCCGACTTCATCTTTAAGGAGTGGAAGAAGCTGGACAAGGCCAAGAA | |
| | AGTGATGGAGAACCAGATGTTCGAGGAGAAGCAGGCCGAGAGCATGCCCGAGATCGAGACAGAGCAGG | |
| | AGTACAAGGAGATTTTCATCACACCTCACCAGATCAAGCACATCAAGGACTTCAAGGACTACAAGTAT | |
| | TCTCACAGGGTGGATAAGAAGCCCAACCGCAAGCTGATCAATGACACCCTGTATAGCACACGGAAGGA | |
| | CGATAAGGGCAATACCCTGATCGTGAACAATCTGAACGGCCTGTACGACAAGGATAATGACAAGCTGA | |
| | AGAAGCTGATCAACAAGTCTCCCGAGAAGCTGCTGATGTACCACCACGATCCTCAGACATATCAGAAG | |
| | CTGAAGCTGATCATGGAGCAGTACGGCGACGAGAAGAACCCACTGTATAAGTACTATGAGGAGACAGG | |
| | CAACTACCTGACAAAGTATAGCAAGAAGGATAATGGCCCCGTGATCAAGAAGATCAAGTACTATGGCA | |
| | ACAAGCTGAATGCCCACCTGGACATCACCGACGATTACCCTAACTCTCGCAATAAGGTGGTGAAGCTG | |
| | AGCCTGAAGCCATACCGGTTCGACGTGTACCTGGACAACGGCGTGTATAAGTTTGTGACAGTGAAGAA | |
| | TCTGGATGTGATCAAGAAGGAGAACTACTATGAGGTGAACAGCAAGTGCTACGAGGAGGCCAAGAAGC | |
| | TGAAGAAGATCAGCAACCAGGCCGAGTTCATCGCCTCTTTTTACAAGAATGACCTGATCAAGATCAAT | |
| | GGCGAGCTGTATAGAGTGATCGGCGTGAACAATGATCTGCTGAACAGAATCGAAGTGAATATGATCGA | |
| | CATCACCTACAGGGAGTATCTGGAGAACATGAATGATAAGAGGCCCCCTCATATCATCAAGACCATCG | |
| | CCTCTAAGACACAGAGCATCAAGAAGTACAGCACAGACATCCTGGGGAACCTGTATGAAGTCAAGAGC | |
| | AAGAAACATCCTCAGATTATCAAGAAAGGCSGGSKRTAEGSEFEPRKKRKV | |
| ABE8-NG editor | NLS, wtTadA, linker, TadA*, NRCH | 470 |
| | Amino Acid Sequence | |
| | MKRTADGSEFESPKKKRKVSEVEFSHEYWMRHALTLAKRAWDEREVPVGAVLVHNNRVIGEGWNRPIG | |
| | RHDPTAHAEIMALRQGGLVMQNYRLIDATLYVTLEPCVMCAGAMIHSRIGRVVFGARDAKTGAAGSLM | |
| | DVLHHPGMNHRVEITEGILADECAALLSDFFRMRRQEIKAQKKAQSSTDSGGSSGGSSGSETPGTSES | |
| | ATPESSGGSSGGSGTTGATGGAGCTCTGGGCCTTCTTCTGAGCATTGAACACCTGTCTAGGCATCCGA | |
| | TAGAAATCGCACAGCAGGGCGGCACATTCATCTGCCAGGATTCCCTCGGTAATTTCGACGCGGTGATT | |
| | CATGCCGGGGTAGTTCAGCACGTTCATCAGGGAGCCTGCGGCGCCTCTTTTTGAGTTCCTCACGCCAA | |
| | ACACCACGCGGCCGATCCTAGAGTGGATCATGGCGCCGGCGCACATCACGCAAGGCTCGAATGTCACG | |
| | TACAGGGTGGCGTCAATCAGTCTGTAGTTCTGCATGACCAGGCCGCCCTGTCTCAGGGCCATAATTTC | |
| | GGCATGGGCTGTTGGGTCGTGCAGGCCGATGGCTCTGTTCCAGCCCTCGCCGATCACTCTATTGTTCA | |
| | GCACCAGCACGGCTCCCACAGGCACCTCCCTCTCATCCCGTGCCCTCTTGGCCAGGGTCAGGGCATGT | |
| | CTCATCCAGTACTCGTGGGAAAACTCCACCTCAGASGGSSGGSSGSETPGTSESATPESSGGSSGGSD | |
| | KKYSIGLAIGTNSVGWAVITDEYKVPSKKFKVLGNTDRHSIKKNLIGALLFDSGEIAEATRLKRTARR | |
| | RYTRRKNRICYLQEIFSNEMAKVDDSFFHRLEESFLVEEDKKHERHPIFGNIVDEVAYHEKYPTIYHL | |
| | RKKLVDSTDKADLRLIYLALAHMIKFRGHFLIEGDLNPDNSDVDKLFIQLVQTYNQLFEENPINASGV | |
| | DAKAILSARLSKSRRLENLIAQLPGEKKNGLFGNLIALSLGLTPNFKSNFDLAEDAKLQLSKDTYDDD | |
| | LDNLLAQIGDQYADLFLAAKNLSDAILLSDILRVNTEITKAPLSASMIKRYDEHHQDLTLLKALVRQQ | |
| | LPEKYKEIFFDQSKNGYAGYIDGGASQEEFYKFIKPILEKMDGTEELLVKLNREDLLRKQRTFDNGSI | |
| | PHQIHLGELHAILRRQEDFYPFLKDNREKIEKILTFRIPYYVGPLARGNSRFAWMTRKSEETITPWNF | |
| | EEVVDKGASAQSFIERMTNFDKNLPNEKVLPKHSLLYEYFTVYNELTKVKYVTEGMRKPAFLSGEQKK | |
| | AIVDLLFKTNRKVTVKQLKEDYFKKIECFDSVEISGVEDRFNASLGTYHDLLKIIKDKDFLDNEENED | |
| | ILEDIVLTLTLFEDREMIEERLKTYAHLFDDKVMKQLKRRRYTGWGRLSRKLINGIRDKQSGKTILDF | |
| | LKSDGFANRNFMQLIHDDSLTFKEDIQKAQVSGQGDSLHEHIANLAGSPAIKKGILQTVKVVDELVKV | |
| | MGRHKPENIVIEMARENQTTQKGQKNSRERMKRIEEGIKELGSQILKEHPVENTQLQNEKLYLYYLQN | |
| | GRDMYVDQELDINRLSDYDVDHIVPQSFLKDDSIDNKVLTRSDKNRGKSDNVPSEEVVKKMKNYWRQL | |
| | LNAKLITQRKFDNLTKAERGGLSELDKAGFIKRQLVETRQITKHVAQILDSRMNTKYDENDKLIREVK | |
| | VITLKSKLVSDFRKDFQFYKVREINNYHHAHDAYLNAVVGTALIKKYPKLESEFVYGDYKVYDVRKMI | |
| | AKSEQEIGKATAKYFFYSNIMNFFKTEITLANGEIRKRPLIETNGETGEIVWDKGRDFATVRKVLSMP | |
| | QVNIVKKTEVQTGGFSKESIRPKRNSDKLIARKKDWDPKKYGGFVSPTVAYSVLVVAKVEKGKSKKLK | |
| | SVKELLGITIMERSSFEKNPIDFLEAKGYKEVKKDLIIKLPKYSLFELENGRKRMLASARFLQKGNEL | |
| | ALPSKYVNFLYLASHYEKLKGSPEDNEQKQLFVEQHKHYLDEIIEQISEFSKRVILADANLDKVLSAY | |
| | NKHRDKPIREQAENIIHLFTLTNLGAPRAFKYFDTTIDRKVYRSTKEVLDATLIHQSITGLYETRIDL | |
| | SQLGGDSGGSKRTADGSEFEPKKKRKV | |
| ABE8-CP1041 | NLS, wtTadA, linker, TadA*, NRCH | 471 |
| editor | Amino Acid Sequence | |
| | MKRTADGSEFESPKKKRKVSEVEFSHEYWMRHALTLAKRAWDEREVPVGAVLVHNNRVIGEGWNRPIG | |
| | RHDPTAHAEIMALRQGGLVMQNYRLIDATLYVTLEPCVMCAGAMIHSRIGRVVFGARDAKTGAAGSLM | |
| | DVLHHPGMNHRVEITEGILADECAALLSDFFRMRRQEIKAQKKAQSSTDSGGSSGGSSGSETPGTSES | |
| | ATPESSGGSSGGSGTTGATGGAGCTCTGGGCCTTCTTCTGAGCATTGAACACCTGTCTAGGCATCCGA | |
| | TAGAAATCGCACAGCAGGGCGGCACATTCATCTGCCAGGATTCCCTCGGTAATTTCGACGCGGTGATT | |
| | CATGCCGGGGTAGTTCAGCACGTTCATCAGGGAGCCTGCGGCGCCTCTTTTTGAGTTCCTCACGCCAA | |
| | ACACCACGCGGCCGATCCTAGAGTGGATCATGGCGCCGGCGCACATCACGCAAGGCTCGAATGTCACG | |
| | TACAGGGTGGCGTCAATCAGTCTGTAGTTCTGCATGACCAGGCCGCCCTGTCTCAGGGCCATAATTTC | |
| | GGCATGGGCTGTTGGGTCGTGCAGGCCGATGGCTCTGTTCCAGCCCTCGCCGATCACTCTATTGTTCA | |
| | GCACCAGCACGGCTCCCACAGGCACCTCCCTCTCATCCCGTGCCCTCTTGGCCAGGGTCAGGGCATGT | |
| | CTCATCCAGTACTCGTGGGAAAACTCCACCTCAGASGGSSGGSSGSETPGTSESATPESSGGSSGGSN | |
| | IMNFFKTEITLANGEIRKRPLIETNGETGEIVWDKGRDFATVRKVLSMPQVNIVKKTEVQTGGFSKES | |
| | ILPKRNSDKLIARKKDWDPKKYGGFDSPTVAYSVLVVAKVEKGKSKKLKSVKELLGITIMERSSFEKN | |
| | PIDFLEAKGYKEVKKDLIIKLPKYSLFELENGRKRMLASAGELQKGNELALPSKYVNFLYLASHYEKL | |
| | KGSPEDNEQKQLFVEQHKHYLDEIIEQISEFSKRVILADANLDKVLSAYNKHRDKPIREQAENIIHLF | |
| | TLTNLGAPAAFKYFDTTIDRKRYTSTKEVLDATLIHQSITGLYETRIDLSQLGGDGGSGGSGGSGGSG | |
| | GSGGSGGDKKYSIGLAIGTNSVGWAVITDEYKVPSKKFKVLGNTDRHSIKKNLIGALLFDSGETAEAT | |
| | RLKRTARRRYTRRKNRICYLQEIFSNEMAKVDDSFFHRLEESFLVEEDKKHERHPIFGNIVDEVAYHE | |
| | KYPTIYHLRKKLVDSTDKADLRLIYLALAHMIKFRGHFLIEGDLNPDNSDVDKLFIQLVQTYNQLFEE | |
| | NPINASGVDAKAILSARLSKSRRLENLIAQLPGEKKNGLFGNLIALSLGLTPNFKSNFDLAEDAKLQL | |
| | SKDTYDDDLDNLLAQIGDQYADLFLAAKNLSDAILLSDILRVNTEITKAPLSASMIKRYDEHHQDLTL | |
| | LKALVRQQLPEKYKEIFFDQSKNGYAGYIDGGASQEEFYKFIKPILEKMDGTEELLVKLNREDLLRKQ | |
| | RTFDNGSIPHQIHLGELHAILRRQEDFYPFLKDNREKIEKILTFRIPYYVGPLARGNSRFAWMTRKSE | |
| | ETITPWNFEEVVDKGASAQSFIERMTNFDKNLPNEKVLPKHSLLYEYFTVYNELTKVKYVTEGMRKPA | |
| | FLSGEQKKAIVDLLFKTNRKVTVKQLKEDYFKKIECFDSVEISGVEDRFNASLGTYHDLLKIIKDKDF | |
| | LDNEENEDILEDIVLTLTLFEDREMIEERLKTYAHLFDDKVMKQLKRRRYTGWGRLSRKLINGIRDKQ | |
| | SGKTILDFLKSDGFANRNFMQLIHDDSLTFKEDIQKAQVSGQGDSLHEHIANLAGSPAIKKGILQTVK | |
| | VVDELVKVMGRHKPENIVIEMARENQTTQKGQKNSRERMKRIEEGIKELGSQILKEHPVENTQLQNEK | |
| | LYLYYLQNGRDMYVDQELDINRLSDYDVDHIVPQSFLKDDSIDNKVLTRSDKNRGKSDNVPSEEVVKK | |
| | MKNYWRQLLNAKLITQRKFDNLTKAERGGLSELDKAGFIKRQLVETRQITKHVAQILDSRMNTKYDEN | |
| | DKLIREVKVITLKSKLVSDFRKDFQFYKVREINNYHHAHDAYLNAVVGTALIKKYPKLESEFVYGDYK | |
| | VYDVRKMIAKSEQEIGKATAKYFFYSSGGSKRTADGSEFEPKKKRKV | |
| ABE8-CP1028 | NLS, wtTadA, linker, TadA*, NRCH | 472 |
| editor | Amino Acid Sequence | |
| | MKRTADGSEFESPKKKRKVSEVEFSHEYWMRHALTLAKRAWDEREVPVGAVLVHNNRVIGEGWNRPIG | |
| | RHDPTAHAEIMALRQGGLVMQNYRLIDATLYVTLEPCVMCAGAMIHSRIGRVVFGARDAKTGAAGSLM | |
| | DVLHHPGMNHRVEITEGILADECAALLSDFFRMRRQEIKAQKKAQSSTDSGGSSGGSSGSETPGTSES | |
| | ATPESSGGSSGGSGTTGATGGAGCTCTGGGCCTTCTTCTGAGCATTGAACACCTGTCTAGGCATCCGA | |
| | TAGAAATCGCACAGCAGGGCGGCACATTCATCTGCCAGGATTCCCTCGGTAATTTCGACGCGGTGATT | |
| | CATGCCGGGGTAGTTCAGCACGTTCATCAGGGAGCCTGCGGCGCCTCTTTTTGAGTTCCTCACGCCAA | |
| | ACACCACGCGGCCGATCCTAGAGTGGATCATGGCGCCGGCGCACATCACGCAAGGCTCGAATGTCACG | |
| | TACAGGGTGGCGTCAATCAGTCTGTAGTTCTGCATGACCAGGCCGCCCTGTCTCAGGGCCATAATTTC | |
| | GGCATGGGCTGTTGGGTCGTGCAGGCCGATGGCTCTGTTCCAGCCCTCGCCGATCACTCTATTGTTCA | |
| | GCACCAGCACGGCTCCCACAGGCACCTCCCTCTCATCCCGTGCCCTCTTGGCCAGGGTCAGGGCATGT | |
| | CTCATCCAGTACTCGTGGGAAAACTCCACCTCAGASGGSSGGSSGSETPGTSESATPESSGGSSGGSE | |
| | IGKATAKYFFYSNIMNFFKTEITLANGEIRKRPLIETNGETGEIVWDKGRDFATVRKVLSMPQVNIVK | |
| | KTEVQTGGFSKESILPKRNSDKLIARKKDWDPKKYGGFDSPTVAYSVLVVAKVEKGKSKKLKSVKELL | |
| | GITIMERSSFEKNPIDFLEAKGYKEVKKDLIIKLPKYSLFELENGRKRMLASAGELQKGNELALPSKY | |
| | VNFLYLASHYEKLKGSPEDNEQKQLFVEQHKHYLDEIIEQISEFSKRVILADANLDKVLSAYNKHRDK | |
| | PIREQAENIIHLFTLTNLGAPAAFKYFDTTIDRKRYTSTKEVLDATLIHQSITGLYETRIDLSQLGGD | |
| | GGSGGSGGSGGSGGSGGSGGMDKKYSIGLAIGTNSVGWAVITDEYKVPSKKFKVLGNTDRHSIKKNLI | |
| | GALLFDSGETAEATRLKRTARRRYTRRKNRICYLQEIFSNEMAKVDDSFFHRLEESFLVEEDKKHERH | |
| | PIFGNIVDEVAYHEKYPTIYHLRKKLVDSTDKADLRLIYLALAHMIKFRGHFLIEGDLNPDNSDVDKL | |
| | FIQLVQTYNQLFEENPINASGVDAKAILSARLSKSRRLENLIAQLPGEKKNGLFGNLIALSLGLTPNF | |
| | KSNFDLAEDAKLQLSKDTYDDDLDNLLAQIGDQYADLFLAAKNLSDAILLSDILRVNTEITKAPLSAS | |
| | MIKRYDEHHQDLTLLKALVRQQLPEKYKEIFFDQSKNGYAGYIDGGASQEEFYKFIKPILEKMDGTEE | |
| | LLVKLNREDLLRKQRTFDNGSIPHQIHLGELHAILRRQEDFYPFLKDNREKIEKILTFRIPYYVGPLA | |
| | RGNSRFAWMTRKSEETITPWNFEEVVDKGASAQSFIERMTNFDKNLPNEKVLPKHSLLYEYFTVYNEL | |
| | TKVKYVTEGMRKPAFLSGEQKKAIVDLLFKTNRKVTVKQLKEDYFKKIECFDSVEISGVEDRFNASLG | |
| | TYHDLLKIIKDKDFLDNEENEDILEDIVLTLTLFEDREMIEERLKTYAHLFDDKVMKQLKRRRYTGWG | |
| | RLSRKLINGIRDKQSGKTILDFLKSDGFANRNFMQLIHDDSLTFKEDIQKAQVSGQGDSLHEHIANLA | |
| | GSPAIKKGILQTVKVVDELVKVMGRHKPENIVIEMARENQTTQKGQKNSRERMKRIEEGIKELGSQIL | |
| | KEHPVENTQLQNEKLYLYYLQNGRDMYVDQELDINRLSDYDVDHIVPQSFLKDDSIDNKVLTRSDKNR | |
| | GKSDNVPSEEVVKKMKNYWRQLLNAKLITQRKFDNLTKAERGGLSELDKAGFIKRQLVETRQITKHVA | |
| | QILDSRMNTKYDENDKLIREVKVITLKSKLVSDFRKDFQFYKVREINNYHHAHDAYLNAWGTALIKK | |
| | YPKLESEFVYGDYKVYDVRKMIAKSEQSGGSKRTADGSEFEPKKKRKV | |
| ABE8-CPF1 | NLS, wtTadA, linker, TadA*, NRCH | 473 |
| editor | Amino Acid Sequence | |
| | MKRTADGSEFESPKKKRKVSEVEFSHEYWMRHALTLAKRAWDEREVPVGAVLVHNNRVIGEGWNRPIG | |
| | RHDPTAHAEIMALRQGGLVMQNYRLIDATLYVTLEPCVMCAGAMIHSRIGRVVFGARDAKTGAAGSLM | |
| | DVLHHPGMNHRVEITEGILADECAALLSDFFRMRRQEIKAQKKAQSSTDSGGSSGGSSGSETPGTSES | |
| | ATPESSGGSSGGSGTTGATGGAGCTCTGGGCCTTCTTCTGAGCATTGAACACCTGTCTAGGCATCCGA | |
| | TAGAAATCGCACAGCAGGGCGGCACATTCATCTGCCAGGATTCCCTCGGTAATTTCGACGCGGTGATT | |
| | CATGCCGGGGTAGTTCAGCACGTTCATCAGGGAGCCTGCGGCGCCTCTTTTTGAGTTCCTCACGCCAA | |
| | ACACCACGCGGCCGATCCTAGAGTGGATCATGGCGCCGGCGCACATCACGCAAGGCTCGAATGTCACG | |
| | TACAGGGTGGCGTCAATCAGTCTGTAGTTCTGCATGACCAGGCCGCCCTGTCTCAGGGCCATAATTTC | |
| | GGCATGGGCTGTTGGGTCGTGCAGGCCGATGGCTCTGTTCCAGCCCTCGCCGATCACTCTATTGTTCA | |
| | GCACCAGCACGGCTCCCACAGGCACCTCCCTCTCATCCCGTGCCCTCTTGGCCAGGGTCAGGGCATGT | |
| | CTCATCCAGTACTCGTGGGAAAACTCCACCTCAGASGGSSGGSSGSETPGTSESATPESSGGSSGGSS | |
| | KLEKFTNCYSLSKTLRFKAIPVGKTQENIDNKRLLVEDEKRAEDYKGVKKLLDRYYLSFINDVLHSIK | |
| | LKNLNNYISLFRKKTRTEKENKELENLEINLRKEIAKAFKGNEGYKSLFKKDIIETILPEFLDDKDEI | |
| | ALVNSFNGFTTAFTGFFDNRENMFSEEAKSTSIAFRCINENLTRYISNMDIFEKVDAIFDKHEVQEIK | |
| | EKILNSDYDVEDFFEGEFFNFVLTQEGIDVYNAIIGGFVTESGEKIKGLNEYINLYNQKTKQKLPKFK | |
| | PLYKQVLSDRESLSFYGEGYTSDEEVLEVFRNTLNKNSEIFSSIKKLEKLFKNFDEYSSAGIFVKNGP | |
| | AISTISKDIFGEWNVIRDKVVNAEYDDIHLKKKAWTEKYEDDRRKSFKKIGSFSLEQLQEYADADLSV | |
| | VEKLKEIIIQKVDEIYKVYGSSEKLFDADFVLEKSLKKNDAVVAIMKDLLDSVKSFENYIKAFFGEGK | |
| | ETNRDESFYGDFVLAYDILLKVDHIYDAIRNYVTQKPYSKDKFKLYFQNPQFMGGWDKDKETDYRATI | |
| | LRYGSKYYLAIMDKKYAKCLQKIDKDDVNGNYEKINYKLLPGPNKMLPKVFFSKKWMAYYNPSEDIQK | |
| | IYKNGTFKKGDMFNLNDCHKLIDFFKDSISRYPKWSNAYDFNFSETEKYKDIAGFYREVEEQGYKVSF | |
| | ESASKKEVDKLVEEGKLYMFQIYNKDFSDKSHGTPNLHTMYFKLLFDENNHGQIRLSGGAELFMRRAS | |
| | LKKEELVVHPANSPIANKNPDNPKKTTTLSYDVYKDKRFSEDQYELHIPIAINKCPKNIFKINTEVRV | |
| | LLKHDDNPYVIGIARGERNLLYIVVVDGKGNIVEQYSLNEIINNFNGIRIKTDYHSLLDKKEKERFEA | |
| | RQNWTSIENIKELKAGYISQVVHKICELVEKYDAVIALEDLNSGFKNSRVKVEKQVYQKFEKMLIDKL | |
| | NYMVDKKSNPCATGGALKGYQITNKFESFKSMSTQNGFIFYIPAWLTSKIDPSTGFVNLLKTKYTSIA | |
| | DSKKFISSFDRIMYVPEEDLFEFALDYKNFSRTDADYIKKWKLYSYGNRIRIFRNPKKNNVFDWEEVC | |
| | LTSAYKELFNKYGINYQQGDIRALLCEQSDKAFYSSFMALMSLMLQMRNSITGRTDVDFLISPVKNSD | |
| | GIFYDSRNYEAQENAILPKNADANGAYNIARKVLWAIGQFKKAEDEKLDKVKIAISNKEWLEYAQTSV | |
| | KSGGSKRTADGSEFEPKKKRKV | |
| ABE8-VRQR | NLS, wtTadA, linker, TadA*, NRCH | 474 |
| editor | Amino Acid Sequence | |
| | MKRTADGSEFESPKKKRKVSEVEFSHEYWMRHALTLAKRAWDEREVPVGAVLVHNNRVIGEGWNRPIG | |
| | RHDPTAHAEIMALRQGGLVMQNYRLIDATLYVTLEPCVMCAGAMIHSRIGRVVFGARDAKTGAAGSLM | |
| | DVLHHPGMNHRVEITEGILADECAALLSDFFRMRRQEIKAQKKAQSSTDSGGSSGGSSGSETPGTSES | |
| | ATPESSGGSSGGSGTTGATGGAGCTCTGGGCCTTCTTCTGAGCATTGAACACCTGTCTAGGCATCCGA | |
| | TAGAAATCGCACAGCAGGGCGGCACATTCATCTGCCAGGATTCCCTCGGTAATTTCGACGCGGTGATT | |
| | CATGCCGGGGTAGTTCAGCACGTTCATCAGGGAGCCTGCGGCGCCTCTTTTTGAGTTCCTCACGCCAA | |
| | ACACCACGCGGCCGATCCTAGAGTGGATCATGGCGCCGGCGCACATCACGCAAGGCTCGAATGTCACG | |
| | TACAGGGTGGCGTCAATCAGTCTGTAGTTCTGCATGACCAGGCCGCCCTGTCTCAGGGCCATAATTTC | |
| | GGCATGGGCTGTTGGGTCGTGCAGGCCGATGGCTCTGTTCCAGCCCTCGCCGATCACTCTATTGTTCA | |
| | GCACCAGCACGGCTCCCACAGGCACCTCCCTCTCATCCCGTGCCCTCTTGGCCAGGGTCAGGGCATGT | |
| | CTCATCCAGTACTCGTGGGAAAACTCCACCTCAGASGGSSGGSSGSETPGTSESATPESSGGSSGGSD | |
| | KKYSIGLAIGTNSVGWAVITDEYKVPSKKFKVLGNTDRHSIKKNLIGALLFDSGETAEATRLKRTARR | |
| | RYTRRKNRICYLQEIFSNEMAKVDDSFFHRLEESFLVEEDKKHERHPIFGNIVDEVAYHEKYPTIYHL | |
| | RKKLVDSTDKADLRLIYLALAHMIKFRGHFLIEGDLNPDNSDVDKLFIQLVQTYNQLFEENPINASGV | |
| | DAKAILSARLSKSRRLENLIAQLPGEKKNGLFGNLIALSLGLTPNFKSNFDLAEDAKLQLSKDTYDDD | |
| | LDNLLAQIGDQYADLFLAAKNLSDAILLSDILRVNTEITKAPLSASMIKRYDEHHQDLTLLKALVRQQ | |
| | LPEKYKEIFFDQSKNGYAGYIDGGASQEEFYKFIKPILEKMDGTEELLVKLNREDLLRKQRTFDNGSI | |
| | PHQIHLGELHAILRRQEDFYPFLKDNREKIEKILTFRIPYYVGPLARGNSRFAWMTRKSEETITPWNF | |
| | EEVVDKGASAQSFIERMTNFDKNLPNEKVLPKHSLLYEYFTVYNELTKVKYVTEGMRKPAFLSGEQKK | |
| | AIVDLLFKTNRKVTVKQLKEDYFKKIECFDSVEISGVEDRFNASLGTYHDLLKIIKDKDFLDNEENED | |
| | ILEDIVLTLTLFEDREMIEERLKTYAHLFDDKVMKQLKRRRYTGWGRLSRKLINGIRDKQSGKTILDF | |
| | LKSDGFANRNFMQLIHDDSLTFKEDIQKAQVSGQGDSLHEHIANLAGSPAIKKGILQTVKVVDELVKV | |
| | MGRHKPENIVIEMARENQTTQKGQKNSRERMKRIEEGIKELGSQILKEHPVENTQLQNEKLYLYYLQN | |
| | GRDMYVDQELDINRLSDYDVDHIVPQSFLKDDSIDNKVLTRSDKNRGKSDNVPSEEVVKKMKNYWRQL | |
| | LNAKLITQRKFDNLTKAERGGLSELDKAGFIKRQLVETRQITKHVAQILDSRMNTKYDENDKLIREVK | |
| | VITLKSKLVSDFRKDFQFYKVREINNYHHAHDAYLNAVVGTALIKKYPKLESEFVYGDYKVYDVRKMI | |
| | AKSEQEIGKATAKYFFYSNIMNFFKTEITLANGEIRKRPLIETNGETGEIVWDKGRDFATVRKVLSMP | |
| | QVNIVKKTEVQTGGFSKESILPKRNSDKLIARKKDWDPKKYGGFVSPTVAYSVLVVAKVEKGKSKKLK | |
| | SVKELLGITIMERSSFEKNPIDFLEAKGYKEVKKDLIIKLPKYSLFELENGRKRMLASARELQKGNEL | |
| | ALPSKYVNFLYLASHYEKLKGSPEDNEQKQLFVEQHKHYLDEIIEQISEFSKRVILADANLDKVLSAY | |
| | NKHRDKPIREQAENIIHLFTLTNLGAPAAFKYFDTTIDRKQYRSTKEVLDATLIHQSITGLYETRIDL | |
| | SQLGGDSGGSKRTADGSEFEPKKKRKV | |
| ABE8-NG-CP1041 | NLS, wtTadA, linker, TadA*, NRCH | 465 |
| editor | Amino Acid Sequence | |
| | MKRTADGSEFESPKKKRKVSEVEFSHEYWMRHALTLAKRAWDEREVPVGAVLVHNNRVIGEGWNRPIG | |
| | RHDPTAHAEIMALRQGGLVMQNYRLIDATLYVTLEPCVMCAGAMIHSRIGRVVFGARDAKTGAAGSLM | |
| | DVLHHPGMNHRVEITEGILADECAALLSDFFRMRRQEIKAQKKAQSSTDSGGSSGGSSGSETPGTSES | |
| | ATPESSGGSSGGSGTTGATGGAGCTCTGGGCCTTCTTCTGAGCATTGAACACCTGTCTAGGCATCCGA | |
| | TAGAAATCGCACAGCAGGGCGGCACATTCATCTGCCAGGATTCCCTCGGTAATTTCGACGCGGTGATT | |
| | CATGCCGGGGTAGTTCAGCACGTTCATCAGGGAGCCTGCGGCGCCTCTTTTTGAGTTCCTCACGCCAA | |
| | ACACCACGCGGCCGATCCTAGAGTGGATCATGGCGCCGGCGCACATCACGCAAGGCTCGAATGTCACG | |
| | TACAGGGTGGCGTCAATCAGTCTGTAGTTCTGCATGACCAGGCCGCCCTGTCTCAGGGCCATAATTTC | |
| | GGCATGGGCTGTTGGGTCGTGCAGGCCGATGGCTCTGTTCCAGCCCTCGCCGATCACTCTATTGTTCA | |
| | GCACCAGCACGGCTCCCACAGGCACCTCCCTCTCATCCCGTGCCCTCTTGGCCAGGGTCAGGGCATGT | |
| | CTCATCCAGTACTCGTGGGAAAACTCCACCTCAGASGGSSGGSSGSETPGTSESATPESSGGSSGGSN | |
| | IMNFFKTEITLANGEIRKRPLIETNGETGEIVWDKGRDFATVRKVLSMPQVNIVKKTEVQTGGFSKES | |
| | IRPKRNSDKLIARKKDWDPKKYGGFVSPTVAYSVLVVAKVEKGKSKKLKSVKELLGITIMERSSFEKN | |
| | PIDFLEAKGYKEVKKDLIIKLPKYSLFELENGRKRMLASARFLQKGNELALPSKYVNFLYLASHYEKL | |
| | KGSPEDNEQKQLFVEQHKHYLDEIIEQISEFSKRVILADANLDKVLSAYNKHRDKPIREQAENIIHLF | |
| | TLTNLGAPRAFKYFDTTIDRKVYRSTKEVLDATLIHQSITGLYETRIDLSQLGGDGGSGGSGGSGGSG | |
| | GSGGSGGDKKYSIGLAIGTNSVGWAVITDEYKVPSKKFKVLGNTDRHSIKKNLIGALLFDSGETAEAT | |
| | RLKRTARRRYTRRKNRICYLQEIFSNEMAKVDDSFFHRLEESFLVEEDKKHERHPIFGNIVDEVAYHE | |
| | KYPTIYHLRKKLVDSTDKADLRLIYLALAHMIKFRGHFLIEGDLNPDNSDVDKLFIQLVQTYNQLFEE | |
| | NPINASGVDAKAILSARLSKSRRLENLIAQLPGEKKNGLFGNLIALSLGLTPNFKSNFDLAEDAKLQL | |
| | SKDTYDDDLDNLLAQIGDQYADLFLAAKNLSDAILLSDILRVNTEITKAPLSASMIKRYDEHHQDLTL | |
| | LKALVRQQLPEKYKEIFFDQSKNGYAGYIDGGASQEEFYKFIKPILEKMDGTEELLVKLNREDLLRKQ | |
| | RTFDNGSIPHQIHLGELHAILRRQEDFYPFLKDNREKIEKILTFRIPYYVGPLARGNSRFAWMTRKSE | |
| | ETITPWNFEEVVDKGASAQSFIERMTNFDKNLPNEKVLPKHSLLYEYFTVYNELTKVKYVTEGMRKPA | |
| | FLSGEQKKAIVDLLFKTNRKVTVKQLKEDYFKKIECFDSVEISGVEDRFNASLGTYHDLLKIIKDKDF | |
| | LDNEENEDILEDIVLTLTLFEDREMIEERLKTYAHLFDDKVMKQLKRRRYTGWGRLSRKLINGIRDKQ | |
| | SGKTILDFLKSDGFANRNFMQLIHDDSLTFKEDIQKAQVSGQGDSLHEHIANLAGSPAIKKGILQTVK | |
| | VVDELVKVMGRHKPENIVIEMARENQTTQKGQKNSRERMKRIEEGIKELGSQILKEHPVENTQLQNEK | |
| | LYLYYLQNGRDMYVDQELDINRLSDYDVDHIVPQSFLKDDSIDNKVLTRSDKNRGKSDNVPSEEVVKK | |
| | MKNYWRQLLNAKLITQRKFDNLTKAERGGLSELDKAGFIKRQLVETRQITKHVAQILDSRMNTKYDEN | |
| | DKLIREVKVITLKSKLVSDFRKDFQFYKVREINNYHHAHDAYLNAWGTALIKKYPKLESEFVYGDYK | |
| | VYDVRKMIAKSEQEIGKATAKYFFYSSGGSKRTADGSEFEPKKKRKV | |
| ABE-SpyMac | NLS, wtTadA, linker, TadA*, NRCH | 476 |
| editor | Amino Acid Sequence | |
| | MKRTADGSEFESPKKKRKVSEVEFSHEYWMRHALTLAKRAWDEREVPVGAVLVHNNRVIGEGWNRPIG | |
| | RHDPTAHAEIMALRQGGLVMQNYRLIDATLYVTLEPCVMCAGAMIHSRIGRVVFGARDAKTGAAGSLM | |
| | DVLHHPGMNHRVEITEGILADECAALLSDFFRMRRQEIKAQKKAQSSTDSGGSSGGSSGSETPGTSES | |
| | ATPESSGGSSGGSGTTGATGGAGCTCTGGGCCTTCTTCTGAGCATTGAACACCTGTCTAGGCATCCGA | |
| | TAGAAATCGCACAGCAGGGCGGCACATTCATCTGCCAGGATTCCCTCGGTAATTTCGACGCGGTGATT | |
| | CATGCCGGGGTAGTTCAGCACGTTCATCAGGGAGCCTGCGGCGCCTCTTTTTGAGTTCCTCACGCCAA | |
| | ACACCACGCGGCCGATCCTAGAGTGGATCATGGCGCCGGCGCACATCACGCAAGGCTCGAATGTCACG | |
| | TACAGGGTGGCGTCAATCAGTCTGTAGTTCTGCATGACCAGGCCGCCCTGTCTCAGGGCCATAATTTC | |
| | GGCATGGGCTGTTGGGTCGTGCAGGCCGATGGCTCTGTTCCAGCCCTCGCCGATCACTCTATTGTTCA | |
| | GCACCAGCACGGCTCCCACAGGCACCTCCCTCTCATCCCGTGCCCTCTTGGCCAGGGTCAGGGCATGT | |
| | CTCATCCAGTACTCGTGGGAAAACTCCACCTCAGASGGSSGGSSGSETPGTSESATPESSGGSSGGSD | |
| | KKYSIGLDIGTNSVGWAVITDEYKVPSKKFKVLGNTDRHSIKKNLIGALLFDSGETAEATRLKRTARR | |
| | RYTRRKNRICYLQEIFSNEMAKVDDSFFHRLEESFLVEEDKKHERHPIFGNIVDEVAYHEKYPTIYHL | |
| | RKKLVDSTDKADLRLIYLALAHMIKFRGHFLIEGDLNPDNSDVDKLFIQLVQTYNQLFEENPINASGV | |
| | DAKAILSARLSKSRRLENLIAQLPGEKKNGLFGNLIALSLGLTPNFKSNFDLAEDAKLQLSKDTYDDD | |
| | LDNLLAQIGDQYADLFLAAKNLSDAILLSDILRVNTEITKAPLSASMIKRYDEHHQDLTLLKALVRQQ | |
| | LPEKYKEIFFDQSKNGYAGYIDGGASQEEFYKFIKPILEKMDGTEELLVKLNREDLLRKQRTFDNGSI | |
| | PHQIHLGELHAILRRQEDFYPFLKDNREKIEKILTFRIPYYVGPLARGNSRFAWMTRKSEETITPWNF | |
| | EEVVDKGASAQSFIERMTNFDKNLPNEKVLPKHSLLYEYFTVYNELTKVKYVTEGMRKPAFLSGEQKK | |
| | AIVDLLFKTNRKVTVKQLKEDYFKKIECFDSVEISGVEDRFNASLGTYHDLLKIIKDKDFLDNEENED | |
| | ILEDIVLTLTLFEDREMIEERLKTYAHLFDDKVMKQLKRRRYTGWGRLSRKLINGIRDKQSGKTILDF | |
| | LKSDGFANRNFMQLIHDDSLTFKEDIQKAQVSGQGDSLHEHIANLAGSPAIKKGILQTVKVVDELVKV | |
| | MGRHKPENIVIEMARENQTTQKGQKNSRERMKRIEEGIKELGSQILKEHPVENTQLQNEKLYLYYLQN | |
| | GRDMYVDQELDINRLSDYDVDHIVPQSFLKDDSIDNKVLTRSDKNRGKSDNVPSEEVVKKMKNYWRQL | |
| | LNAKLITQRKFDNLTKAERGGLSELDKAGFIKRQLVETRQITKHVAQILDSRMNTKYDENDKLIREVK | |
| | VITLKSKLVSDFRKDFQFYKVREINNYHHAHDAYLNAVVGTALIKKYPKLESEFVYGDYKVYDVRKMI | |
| | AKSEQEIGKATAKYFFYSNIMNFFKTEITLANGEIRKRPLIETNGETGEIVWDKGRDFATVRKVLSMP | |
| | QVNIVKKTESGGSKRTADGSEFEPKKKRKV | |

CBEs (cytosine base editors)

| | | SEQ ID |
|---|---|---|
| DESCRIPTION | SEQUENCE | NO: |
| BE4max | MKRTADGSEFESPKKKRKVSSETGPVAVDPTLRRRIEPHEFEVFFDPRELRKETCLLYEINWGGRHSI | 223 |
| | WRHTSQNTNKHVEVNFIEKFTTERYFCPNTRCSITWFLSWSPCGECSRAITEFLSRYPHVTLFIYIAR | |
| | LYHHADPRNRQGLRDLISSGVTIQIMTEQESGYCWRNFVNYSPSNEAHWPRYPHLWVRLYVLELYCII | |
| | LGLPPCLNILRRKQPQLTFFTIALQSCHYQRLPPHILWATGLKSGGSSGGSSGSETPGTSESATPESS | |
| | GGSSGGSDKKYSIGLAIGTNSVGWAVITDEYKVPSKKFKVLGNTDRHSIKKNLIGALLFDSGETAEAT | |
| | RLKRTARRRYTRRKNRICYLQEIFSNEMAKVDDSFFHRLEESFLVEEDKKHERHPIFGNIVDEVAYHE | |
| | KYPTIYHLRKKLVDSTDKADLRLIYLALAHMIKFRGHFLIEGDLNPDNSDVDKLFIQLVQTYNQLFEE | |
| | NPINASGVDAKAILSARLSKSRRLENLIAQLPGEKKNGLFGNLIALSLGLTPNFKSNFDLAEDAKLQL | |
| | SKDTYDDDLDNLLAQIGDQYADLFLAAKNLSDAILLSDILRVNTEITKAPLSASMIKRYDEHHQDLTL | |
| | LKALVRQQLPEKYKEIFFDQSKNGYAGYIDGGASQEEFYKFIKPILEKMDGTEELLVKLNREDLLRKQ | |
| | RTFDNGSIPHQIHLGELHAILRRQEDFYPFLKDNREKIEKILTFRIPYYVGPLARGNSRFAWMTRKSE | |
| | ETITPWNFEEVVDKGASAQSFIERMTNFDKNLPNEKVLPKHSLLYEYFTVYNELTKVKYVTEGMRKPA | |
| | FLSGEQKKAIVDLLFKTNRKVTVKQLKEDYFKKIECFDSVEISGVEDRFNASLGTYHDLLKIIKDKDF | |
| | LDNEENEDILEDIVLTLTLFEDREMIEERLKTYAHLFDDKVMKQLKRRRYTGWGRLSRKLINGIRDKQ | |
| | SGKTILDFLKSDGFANRNFMQLIHDDSLTFKEDIQKAQVSGQGDSLHEHIANLAGSPAIKKGILQTVK | |
| | VVDELVKVMGRHKPENIVIEMARENQTTQKGQKNSRERMKRIEEGIKELGSQILKEHPVENTQLQNEK | |
| | LYLYYLQNGRDMYVDQELDINRLSDYDVDHIVPQSFLKDDSIDNKVLTRSDKNRGKSDNVPSEEVVKK | |
| | MKNYWRQLLNAKLITQRKFDNLTKAERGGLSELDKAGFIKRQLVETRQITKHVAQILDSRMNTKYDEN | |
| | DKLIREVKVITLKSKLVSDFRKDFQFYKVREINNYHHAHDAYLNAVVGTALIKKYPKLESEFVYGDYK | |
| | VYDVRKMIAKSEQEIGKATAKYFFYSNIMNFFKTEITLANGEIRKRPLIETNGETGEIVWDKGRDFAT | |
| | VRKVLSMPQVNIVKKTEVQTGGFSKESILPKRNSDKLIARKKDWDPKKYGGFDSPTVAYSVLVVAKVE | |
| | KGKSKKLKSVKELLGITIMERSSFEKNPIDFLEAKGYKEVKKDLIIKLPKYSLFELENGRKRMLASAG | |
| | ELQKGNELALPSKYVNFLYLASHYEKLKGSPEDNEQKQLFVEQHKHYLDEIIEQISEFSKRVILADAN | |
| | LDKVLSAYNKHRDKPIREQAENIIHLFTLTNLGAPAAFKYFDTTIDRKRYTSTKEVLDATLIHQSITG | |
| | LYETRIDLSQLGGDSGGSGGSGGSTNLSDIIEKETGKQLVIQESILMLPEEVEEVIGNKPESDILVHT | |
| | AYDESTDENVMLLTSDAPEYKPWALVIQDSNGENKIKMLSGGSGGSGGSTNLSDIIEKETGKQLVIQE | |
| | SILMLPEEVEEVIGNKPESDILVHTAYDESTDENVMLLTSDAPEYKPWALVIQDSNGENKIKMLSGGS | |
| | KRTADGSEFEPKKKRKV | |
| YE1-BE4 | MKRTADGSEFESPKKKRKVSSETGPVAVDPTLRRRIEPHEFEVFFDPRELRKETCLLYEINWGGRHSI | 224 |
| | WRHTSQNTNKHVEVNFIEKFTTERYFCPNTRCSITWFLSYSPCGECSRAITEFLSRYPHVTLFIYIAR | |
| | LYHHADPENRQGLRDLISSGVTIQIMTEQESGYCWRNFVNYSPSNEAHWPRYPHLWVRLYVLELYCII | |
| | LGLPPCLNILRRKQPQLTFFTIALQSCHYQRLPPHILWATGLKSGGSSGGSSGSETPGTSESATPESS | |
| | GGSSGGSDKKYSIGLAIGTNSVGWAVITDEYKVPSKKFKVLGNTDRHSIKKNLIGALLFDSGETAEAT | |
| | RLKRTARRRYTRRKNRICYLQEIFSNEMAKVDDSFFHRLEESFLVEEDKKHERHPIFGNIVDEVAYHE | |
| | KYPTIYHLRKKLVDSTDKADLRLIYLALAHMIKFRGHFLIEGDLNPDNSDVDKLFIQLVQTYNQLFEE | |
| | NPINASGVDAKAILSARLSKSRRLENLIAQLPGEKKNGLFGNLIALSLGLTPNFKSNFDLAEDAKLQL | |
| | SKDTYDDDLDNLLAQIGDQYADLFLAAKNLSDAILLSDILRVNTEITKAPLSASMIKRYDEHHQDLTL | |
| | LKALVRQQLPEKYKEIFFDQSKNGYAGYIDGGASQEEFYKFIKPILEKMDGTEELLVKLNREDLLRKQ | |
| | RTFDNGSIPHQIHLGELHAILRRQEDFYPFLKDNREKIEKILTFRIPYYVGPLARGNSRFAWMTRKSE | |
| | ETITPWNFEEVVDKGASAQSFIERMTNFDKNLPNEKVLPKHSLLYEYFTVYNELTKVKYVTEGMRKPA | |
| | FLSGEQKKAIVDLLFKTNRKVTVKQLKEDYFKKIECFDSVEISGVEDRFNASLGTYHDLLKIIKDKDF | |
| | LDNEENEDILEDIVLTLTLFEDREMIEERLKTYAHLFDDKVMKQLKRRRYTGWGRLSRKLINGIRDKQ | |
| | SGKTILDFLKSDGFANRNFMQLIHDDSLTFKEDIQKAQVSGQGDSLHEHIANLAGSPAIKKGILQTVK | |
| | VVDELVKVMGRHKPENIVIEMARENQTTQKGQKNSRERMKRIEEGIKELGSQILKEHPVENTQLQNEK | |
| | LYLYYLQNGRDMYVDQELDINRLSDYDVDHIVPQSFLKDDSIDNKVLTRSDKNRGKSDNVPSEEVVKK | |
| | MKNYWRQLLNAKLITQRKFDNLTKAERGGLSELDKAGFIKRQLVETRQITKHVAQILDSRMNTKYDEN | |
| | DKLIREVKVITLKSKLVSDFRKDFQFYKVREINNYHHAHDAYLNAVVGTALIKKYPKLESEFVYGDYK | |
| | VYDVRKMIAKSEQEIGKATAKYFFYSNIMNFFKTEITLANGEIRKRPLIETNGETGEIVWDKGRDFAT | |
| | VRKVLSMPQVNIVKKTEVQTGGFSKESILPKRNSDKLIARKKDWDPKKYGGFDSPTVAYSVLVVAKVE | |
| | KGKSKKLKSVKELLGITIMERSSFEKNPIDFLEAKGYKEVKKDLIIKLPKYSLFELENGRKRMLASAG | |
| | ELQKGNELALPSKYVNFLYLASHYEKLKGSPEDNEQKQLFVEQHKHYLDEIIEQISEFSKRVILADAN | |
| | LDKVLSAYNKHRDKPIREQAENIIHLFTLTNLGAPAAFKYFDTTIDRKRYTSTKEVLDATLIHQSITG | |
| | LYETRIDLSQLGGDSGGSGGSGGSTNLSDIIEKETGKQLVIQESILMLPEEVEEVIGNKPESDILVHT | |
| | AYDESTDENVMLLTSDAPEYKPWALVIQDSNGENKIKMLSGGSGGSGGSTNLSDIIEKETGKQLVIQE | |
| | SILMLPEEVEEVIGNKPESDILVHTAYDESTDENVMLLTSDAPEYKPWALVIQDSNGENKIKMLSGGS | |
| | KRTADGSEFEPKKKRKV | |
| YE2-BE4 | MKRTADGSEFESPKKKRKVSSETGPVAVDPTLRRRIEPHEFEVFFDPRELRKETCLLYEINWGGRHSI | 225 |
| | WRHTSQNTNKHVEVNFIEKFTTERYFCPNTRCSITWFLSYSPCGECSRAITEFLSRYPHVTLFIYIAR | |
| | LYHHADPRNRQGLEDLISSGVTIQIMTEQESGYCWRNFVNYSPSNEAHWPRYPHLWVRLYVLELYCII | |
| | LGLPPCLNILRRKQPQLTFFTIALQSCHYQRLPPHILWATGLKSGGSSGGSSGSETPGTSESATPESS | |
| | GGSSGGSDKKYSIGLAIGTNSVGWAVITDEYKVPSKKFKVLGNTDRHSIKKNLIGALLFDSGETAEAT | |
| | RLKRTARRRYTRRKNRICYLQEIFSNEMAKVDDSFFHRLEESFLVEEDKKHERHPIFGNIVDEVAYHE | |
| | KYPTIYHLRKKLVDSTDKADLRLIYLALAHMIKFRGHFLIEGDLNPDNSDVDKLFIQLVQTYNQLFEE | |
| | NPINASGVDAKAILSARLSKSRRLENLIAQLPGEKKNGLFGNLIALSLGLTPNFKSNFDLAEDAKLQL | |
| | SKDTYDDDLDNLLAQIGDQYADLFLAAKNLSDAILLSDILRVNTEITKAPLSASMIKRYDEHHQDLTL | |
| | LKALVRQQLPEKYKEIFFDQSKNGYAGYIDGGASQEEFYKFIKPILEKMDGTEELLVKLNREDLLRKQ | |
| | RTFDNGSIPHQIHLGELHAILRRQEDFYPFLKDNREKIEKILTFRIPYYVGPLARGNSRFAWMTRKSE | |
| | ETITPWNFEEVVDKGASAQSFIERMTNFDKNLPNEKVLPKHSLLYEYFTVYNELTKVKYVTEGMRKPA | |
| | FLSGEQKKAIVDLLFKTNRKVTVKQLKEDYFKKIECFDSVEISGVEDRFNASLGTYHDLLKIIKDKDF | |
| | LDNEENEDILEDIVLTLTLFEDREMIEERLKTYAHLFDDKVMKQLKRRRYTGWGRLSRKLINGIRDKQ | |
| | SGKTILDFLKSDGFANRNFMQLIHDDSLTFKEDIQKAQVSGQGDSLHEHIANLAGSPAIKKGILQTVK | |
| | VVDELVKVMGRHKPENIVIEMARENQTTQKGQKNSRERMKRIEEGIKELGSQILKEHPVENTQLQNEK | |
| | LYLYYLQNGRDMYVDQELDINRLSDYDVDHIVPQSFLKDDSIDNKVLTRSDKNRGKSDNVPSEEVVKK | |
| | MKNYWRQLLNAKLITQRKFDNLTKAERGGLSELDKAGFIKRQLVETRQITKHVAQILDSRMNTKYDEN | |
| | DKLIREVKVITLKSKLVSDFRKDFQFYKVREINNYHHAHDAYLNAVVGTALIKKYPKLESEFVYGDYK | |
| | VYDVRKMIAKSEQEIGKATAKYFFYSNIMNFFKTEITLANGEIRKRPLIETNGETGEIVWDKGRDFAT | |
| | VRKVLSMPQVNIVKKTEVQTGGFSKESILPKRNSDKLIARKKDWDPKKYGGFDSPTVAYSVLVVAKVE | |
| | KGKSKKLKSVKELLGITIMERSSFEKNPIDFLEAKGYKEVKKDLIIKLPKYSLFELENGRKRMLASAG | |
| | ELQKGNELALPSKYVNFLYLASHYEKLKGSPEDNEQKQLFVEQHKHYLDEIIEQISEFSKRVILADAN | |
| | LDKVLSAYNKHRDKPIREQAENIIHLFTLTNLGAPAAFKYFDTTIDRKRYTSTKEVLDATLIHQSITG | |
| | LYETRIDLSQLGGDSGGSGGSGGSTNLSDIIEKETGKQLVIQESILMLPEEVEEVIGNKPESDILVHT | |
| | AYDESTDENVMLLTSDAPEYKPWALVIQDSNGENKIKMLSGGSGGSGGSTNLSDIIEKETGKQLVIQE | |
| | SILMLPEEVEEVIGNKPESDILVHTAYDESTDENVMLLTSDAPEYKPWALVIQDSNGENKIKMLSGGS | |
| | KRTADGSEFEPKKKRKV | |
| YEE-BE4 | MKRTADGSEFESPKKKRKVSSETGPVAVDPTLRRRIEPHEFEVFFDPRELRKETCLLYEINWGGRHSI | 226 |
| | WRHTSQNTNKHVEVNFIEKFTTERYFCPNTRCSITWFLSYSPCGECSRAITEFLSRYPHVTLFIYIAR | |
| | LYHHADPENRQGLEDLISSGVTIQIMTEQESGYCWRNFVNYSPSNEAHWPRYPHLWVRLYVLELYCII | |
| | LGLPPCLNILRRKQPQLTFFTIALQSCHYQRLPPHILWATGLKSGGSSGGSSGSETPGTSESATPESS | |
| | GGSSGGSDKKYSIGLAIGTNSVGWAVITDEYKVPSKKFKVLGNTDRHSIKKNLIGALLFDSGETAEAT | |
| | RLKRTARRRYTRRKNRICYLQEIFSNEMAKVDDSFFHRLEESFLVEEDKKHERHPIFGNIVDEVAYHE | |
| | KYPTIYHLRKKLVDSTDKADLRLIYLALAHMIKFRGHFLIEGDLNPDNSDVDKLFIQLVQTYNQLFEE | |
| | NPINASGVDAKAILSARLSKSRRLENLIAQLPGEKKNGLFGNLIALSLGLTPNFKSNFDLAEDAKLQL | |
| | SKDTYDDDLDNLLAQIGDQYADLFLAAKNLSDAILLSDILRVNTEITKAPLSASMIKRYDEHHQDLTL | |
| | LKALVRQQLPEKYKEIFFDQSKNGYAGYIDGGASQEEFYKFIKPILEKMDGTEELLVKLNREDLLRKQ | |
| | RTFDNGSIPHQIHLGELHAILRRQEDFYPFLKDNREKIEKILTFRIPYYVGPLARGNSRFAWMTRKSE | |
| | ETITPWNFEEVVDKGASAQSFIERMTNFDKNLPNEKVLPKHSLLYEYFTVYNELTKVKYVTEGMRKPA | |
| | FLSGEQKKAIVDLLFKTNRKVTVKQLKEDYFKKIECFDSVEISGVEDRFNASLGTYHDLLKIIKDKDF | |
| | LDNEENEDILEDIVLTLTLFEDREMIEERLKTYAHLFDDKVMKQLKRRRYTGWGRLSRKLINGIRDKQ | |
| | SGKTILDFLKSDGFANRNFMQLIHDDSLTFKEDIQKAQVSGQGDSLHEHIANLAGSPAIKKGILQTVK | |
| | VVDELVKVMGRHKPENIVIEMARENQTTQKGQKNSRERMKRIEEGIKELGSQILKEHPVENTQLQNEK | |
| | LYLYYLQNGRDMYVDQELDINRLSDYDVDHIVPQSFLKDDSIDNKVLTRSDKNRGKSDNVPSEEVVKK | |
| | MKNYWRQLLNAKLITQRKFDNLTKAERGGLSELDKAGFIKRQLVETRQITKHVAQILDSRMNTKYDEN | |
| | DKLIREVKVITLKSKLVSDFRKDFQFYKVREINNYHHAHDAYLNAVVGTALIKKYPKLESEFVYGDYK | |
| | VYDVRKMIAKSEQEIGKATAKYFFYSNIMNFFKTEITLANGEIRKRPLIETNGETGEIVWDKGRDFAT | |
| | VRKVLSMPQVNIVKKTEVQTGGFSKESILPKRNSDKLIARKKDWDPKKYGGFDSPTVAYSVLVVAKVE | |
| | KGKSKKLKSVKELLGITIMERSSFEKNPIDFLEAKGYKEVKKDLIIKLPKYSLFELENGRKRMLASAG | |
| | ELQKGNELALPSKYVNFLYLASHYEKLKGSPEDNEQKQLFVEQHKHYLDEIIEQISEFSKRVILADAN | |
| | LDKVLSAYNKHRDKPIREQAENIIHLFTLTNLGAPAAFKYFDTTIDRKRYTSTKEVLDATLIHQSITG | |
| | LYETRIDLSQLGGDSGGSGGSGGSTNLSDIIEKETGKQLVIQESILMLPEEVEEVIGNKPESDILVHT | |
| | AYDESTDENVMLLTSDAPEYKPWALVIQDSNGENKIKMLSGGSGGSGGSTNLSDIIEKETGKQLVIQE | |
| | SILMLPEEVEEVIGNKPESDILVHTAYDESTDENVMLLTSDAPEYKPWALVIQDSNGENKIKMLSGGS | |
| | KRTADGSEFEPKKKRKV | |
| EE-BE4 | MKRTADGSEFESPKKKRKVSSETGPVAVDPTLRRRIEPHEFEVFFDPRELRKETCLLYEINWGGRHSI | 227 |
| | WRHTSQNTNKHVEVNFIEKFTTERYFCPNTRCSITWFLSWSPCGECSRAITEFLSRYPHVTLFIYIAR | |
| | LYHHADPENRQGLEDLISSGVTIQIMTEQESGYCWRNFVNYSPSNEAHWPRYPHLWVRLYVLELYCII | |
| | LGLPPCLNILRRKQPQLTFFTIALQSCHYQRLPPHILWATGLKSGGSSGGSSGSETPGTSESATPESS | |
| | GGSSGGSDKKYSIGLAIGTNSVGWAVITDEYKVPSKKFKVLGNTDRHSIKKNLIGALLFDSGETAEAT | |
| | RLKRTARRRYTRRKNRICYLQEIFSNEMAKVDDSFFHRLEESFLVEEDKKHERHPIFGNIVDEVAYHE | |
| | KYPTIYHLRKKLVDSTDKADLRLIYLALAHMIKFRGHFLIEGDLNPDNSDVDKLFIQLVQTYNQLFEE | |
| | NPINASGVDAKAILSARLSKSRRLENLIAQLPGEKKNGLFGNLIALSLGLTPNFKSNFDLAEDAKLQL | |
| | SKDTYDDDLDNLLAQIGDQYADLFLAAKNLSDAILLSDILRVNTEITKAPLSASMIKRYDEHHQDLTL | |
| | LKALVRQQLPEKYKEIFFDQSKNGYAGYIDGGASQEEFYKFIKPILEKMDGTEELLVKLNREDLLRKQ | |
| | RTFDNGSIPHQIHLGELHAILRRQEDFYPFLKDNREKIEKILTFRIPYYVGPLARGNSRFAWMTRKSE | |
| | ETITPWNFEEVVDKGASAQSFIERMTNFDKNLPNEKVLPKHSLLYEYFTVYNELTKVKYVTEGMRKPA | |
| | FLSGEQKKAIVDLLFKTNRKVTVKQLKEDYFKKIECFDSVEISGVEDRFNASLGTYHDLLKIIKDKDF | |
| | LDNEENEDILEDIVLTLTLFEDREMIEERLKTYAHLFDDKVMKQLKRRRYTGWGRLSRKLINGIRDKQ | |
| | SGKTILDFLKSDGFANRNFMQLIHDDSLTFKEDIQKAQVSGQGDSLHEHIANLAGSPAIKKGILQTVK | |
| | VVDELVKVMGRHKPENIVIEMARENQTTQKGQKNSRERMKRIEEGIKELGSQILKEHPVENTQLQNEK | |
| | LYLYYLQNGRDMYVDQELDINRLSDYDVDHIVPQSFLKDDSIDNKVLTRSDKNRGKSDNVPSEEVVKK | |
| | MKNYWRQLLNAKLITQRKFDNLTKAERGGLSELDKAGFIKRQLVETRQITKHVAQILDSRMNTKYDEN | |
| | DKLIREVKVITLKSKLVSDFRKDFQFYKVREINNYHHAHDAYLNAVVGTALIKKYPKLESEFVYGDYK | |
| | VYDVRKMIAKSEQEIGKATAKYFFYSNIMNFFKTEITLANGEIRKRPLIETNGETGEIVWDKGRDFAT | |
| | VRKVLSMPQVNIVKKTEVQTGGFSKESILPKRNSDKLIARKKDWDPKKYGGFDSPTVAYSVLVVAKVE | |
| | KGKSKKLKSVKELLGITIMERSSFEKNPIDFLEAKGYKEVKKDLIIKLPKYSLFELENGRKRMLASAG | |
| | ELQKGNELALPSKYVNFLYLASHYEKLKGSPEDNEQKQLFVEQHKHYLDEIIEQISEFSKRVILADAN | |
| | LDKVLSAYNKHRDKPIREQAENIIHLFTLTNLGAPAAFKYFDTTIDRKRYTSTKEVLDATLIHQSITG | |
| | LYETRIDLSQLGGDSGGSGGSGGSTNLSDIIEKETGKQLVIQESILMLPEEVEEVIGNKPESDILVHT | |
| | AYDESTDENVMLLTSDAPEYKPWALVIQDSNGENKIKMLSGGSGGSGGSTNLSDIIEKETGKQLVIQE | |
| | SILMLPEEVEEVIGNKPESDILVHTAYDESTDENVMLLTSDAPEYKPWALVIQDSNGENKIKMLSGGS | |
| | KRTADGSEFEPKKKRKV | |
| R33A-BE4 | MKRTADGSEFESPKKKRKVSSETGPVAVDPTLRRRIEPHEFEVFFDPRELAKETCLLYEINWGGRHSI | 228 |
| | WRHTSQNTNKHVEVNFIEKFTTERYFCPNTRCSITWFLSWSPCGECSRAITEFLSRYPHVTLFIYIAR | |
| | LYHHADPRNRQGLRDLISSGVTIQIMTEQESGYCWRNFVNYSPSNEAHWPRYPHLWVRLYVLELYCII | |
| | LGLPPCLNILRRKQPQLTFFTIALQSCHYQRLPPHILWATGLKSGGSSGGSSGSETPGTSESATPESS | |
| | GGSSGGSDKKYSIGLAIGTNSVGWAVITDEYKVPSKKFKVLGNTDRHSIKKNLIGALLFDSGETAEAT | |
| | RLKRTARRRYTRRKNRICYLQEIFSNEMAKVDDSFFHRLEESFLVEEDKKHERHPIFGNIVDEVAYHE | |
| | KYPTIYHLRKKLVDSTDKADLRLIYLALAHMIKFRGHFLIEGDLNPDNSDVDKLFIQLVQTYNQLFEE | |
| | NPINASGVDAKAILSARLSKSRRLENLIAQLPGEKKNGLFGNLIALSLGLTPNFKSNFDLAEDAKLQL | |
| | SKDTYDDDLDNLLAQIGDQYADLFLAAKNLSDAILLSDILRVNTEITKAPLSASMIKRYDEHHQDLTL | |
| | LKALVRQQLPEKYKEIFFDQSKNGYAGYIDGGASQEEFYKFIKPILEKMDGTEELLVKLNREDLLRKQ | |
| | RTFDNGSIPHQIHLGELHAILRRQEDFYPFLKDNREKIEKILTFRIPYYVGPLARGNSRFAWMTRKSE | |
| | ETITPWNFEEVVDKGASAQSFIERMTNFDKNLPNEKVLPKHSLLYEYFTVYNELTKVKYVTEGMRKPA | |
| | FLSGEQKKAIVDLLFKTNRKVTVKQLKEDYFKKIECFDSVEISGVEDRFNASLGTYHDLLKIIKDKDF | |
| | LDNEENEDILEDIVLTLTLFEDREMIEERLKTYAHLFDDKVMKQLKRRRYTGWGRLSRKLINGIRDKQ | |
| | SGKTILDFLKSDGFANRNFMQLIHDDSLTFKEDIQKAQVSGQGDSLHEHIANLAGSPAIKKGILQTVK | |
| | VVDELVKVMGRHKPENIVIEMARENQTTQKGQKNSRERMKRIEEGIKELGSQILKEHPVENTQLQNEK | |
| | LYLYYLQNGRDMYVDQELDINRLSDYDVDHIVPQSFLKDDSIDNKVLTRSDKNRGKSDNVPSEEVVKK | |
| | MKNYWRQLLNAKLITQRKFDNLTKAERGGLSELDKAGFIKRQLVETRQITKHVAQILDSRMNTKYDEN | |
| | DKLIREVKVITLKSKLVSDFRKDFQFYKVREINNYHHAHDAYLNAVVGTALIKKYPKLESEFVYGDYK | |
| | VYDVRKMIAKSEQEIGKATAKYFFYSNIMNFFKTEITLANGEIRKRPLIETNGETGEIVWDKGRDFAT | |
| | VRKVLSMPQVNIVKKTEVQTGGFSKESILPKRNSDKLIARKKDWDPKKYGGFDSPTVAYSVLVVAKVE | |
| | KGKSKKLKSVKELLGITIMERSSFEKNPIDFLEAKGYKEVKKDLIIKLPKYSLFELENGRKRMLASAG | |
| | ELQKGNELALPSKYVNFLYLASHYEKLKGSPEDNEQKQLFVEQHKHYLDEIIEQISEFSKRVILADAN | |
| | LDKVLSAYNKHRDKPIREQAENIIHLFTLTNLGAPAAFKYFDTTIDRKRYTSTKEVLDATLIHQSITG | |
| | LYETRIDLSQLGGDSGGSGGSGGSTNLSDIIEKETGKQLVIQESILMLPEEVEEVIGNKPESDILVHT | |
| | AYDESTDENVMLLTSDAPEYKPWALVIQDSNGENKIKMLSGGSGGSGGSTNLSDIIEKETGKQLVIQE | |
| | SILMLPEEVEEVIGNKPESDILVHTAYDESTDENVMLLTSDAPEYKPWALVIQDSNGENKIKMLSGGS | |
| | KRTADGSEFEPKKKRKV | |
| R33A + K34A-BE4 | MKRTADGSEFESPKKKRKVSSETGPVAVDPTLRRRIEPHEFEVFFDPRELAAETCLLYEINWGGRHSI | 229 |
| | WRHTSQNTNKHVEVNFIEKFTTERYFCPNTRCSITWFLSWSPCGECSRAITEFLSRYPHVTLFIYIAR | |
| | LYHHADPRNRQGLRDLISSGVTIQIMTEQESGYCWRNFVNYSPSNEAHWPRYPHLWVRLYVLELYCII | |
| | LGLPPCLNILRRKQPQLTFFTIALQSCHYQRLPPHILWATGLKSGGSSGGSSGSETPGTSESATPESS | |
| | GGSSGGSDKKYSIGLAIGTNSVGWAVITDEYKVPSKKFKVLGNTDRHSIKKNLIGALLFDSGETAEAT | |
| | RLKRTARRRYTRRKNRICYLQEIFSNEMAKVDDSFFHRLEESFLVEEDKKHERHPIFGNIVDEVAYHE | |
| | KYPTIYHLRKKLVDSTDKADLRLIYLALAHMIKFRGHFLIEGDLNPDNSDVDKLFIQLVQTYNQLFEE | |
| | NPINASGVDAKAILSARLSKSRRLENLIAQLPGEKKNGLFGNLIALSLGLTPNFKSNFDLAEDAKLQL | |
| | SKDTYDDDLDNLLAQIGDQYADLFLAAKNLSDAILLSDILRVNTEITKAPLSASMIKRYDEHHQDLTL | |
| | LKALVRQQLPEKYKEIFFDQSKNGYAGYIDGGASQEEFYKFIKPILEKMDGTEELLVKLNREDLLRKQ | |
| | RTFDNGSIPHQIHLGELHAILRRQEDFYPFLKDNREKIEKILTFRIPYYVGPLARGNSRFAWMTRKSE | |
| | ETITPWNFEEVVDKGASAQSFIERMTNFDKNLPNEKVLPKHSLLYEYFTVYNELTKVKYVTEGMRKPA | |
| | FLSGEQKKAIVDLLFKTNRKVTVKQLKEDYFKKIECFDSVEISGVEDRFNASLGTYHDLLKIIKDKDF | |
| | LDNEENEDILEDIVLTLTLFEDREMIEERLKTYAHLFDDKVMKQLKRRRYTGWGRLSRKLINGIRDKQ | |
| | SGKTILDFLKSDGFANRNFMQLIHDDSLTFKEDIQKAQVSGQGDSLHEHIANLAGSPAIKKGILQTVK | |
| | VVDELVKVMGRHKPENIVIEMARENQTTQKGQKNSRERMKRIEEGIKELGSQILKEHPVENTQLQNEK | |
| | LYLYYLQNGRDMYVDQELDINRLSDYDVDHIVPQSFLKDDSIDNKVLTRSDKNRGKSDNVPSEEVVKK | |
| | MKNYWRQLLNAKLITQRKFDNLTKAERGGLSELDKAGFIKRQLVETRQITKHVAQILDSRMNTKYDEN | |
| | DKLIREVKVITLKSKLVSDFRKDFQFYKVREINNYHHAHDAYLNAVVGTALIKKYPKLESEFVYGDYK | |
| | VYDVRKMIAKSEQEIGKATAKYFFYSNIMNFFKTEITLANGEIRKRPLIETNGETGEIVWDKGRDFAT | |
| | VRKVLSMPQVNIVKKTEVQTGGFSKESILPKRNSDKLIARKKDWDPKKYGGFDSPTVAYSVLVVAKVE | |
| | KGKSKKLKSVKELLGITIMERSSFEKNPIDFLEAKGYKEVKKDLIIKLPKYSLFELENGRKRMLASAG | |
| | ELQKGNELALPSKYVNFLYLASHYEKLKGSPEDNEQKQLFVEQHKHYLDEIIEQISEFSKRVILADAN | |
| | LDKVLSAYNKHRDKPIREQAENIIHLFTLTNLGAPAAFKYFDTTIDRKRYTSTKEVLDATLIHQSITG | |
| | LYETRIDLSQLGGDSGGSGGSGGSTNLSDIIEKETGKQLVIQESILMLPEEVEEVIGNKPESDILVHT | |
| | AYDESTDENVMLLTSDAPEYKPWALVIQDSNGENKIKMLSGGSGGSGGSTNLSDIIEKETGKQLVIQE | |
| | SILMLPEEVEEVIGNKPESDILVHTAYDESTDENVMLLTSDAPEYKPWALVIQDSNGENKIKMLSGGS | |
| | KRTADGSEFEPKKKRKV | |
| APOBEC3A | MKRTADGSEFESPKKKRKVSEASPASGPRHLMDPHIFTSNFNNGIGRHKTYLCYEVERLDNGTSVKMD | 230 |
| (A3A)-BE4 | QHRGFLHNQAKNLLCGFYGRHAELRFLDLVPSLQLDPAQIYRVTWFISWSPCFSWGCAGEVRAFLQEN | |
| | THVRLRIFAARIYDYDPLYKEALQMLRDAGAQVSIMTYDEFKHCWDTFVDHQGCPFQPWDGLDEHSQA | |
| | LSGRLRAILQNQGNSGGSSGGSSGSETPGTSESATPESSGGSSGGSDKKYSIGLAIGTNSVGWAVITD | |
| | EYKVPSKKFKVLGNTDRHSIKKNLIGALLFDSGETAEATRLKRTARRRYTRRKNRICYLQEIFSNEMA | |
| | KVDDSFFHRLEESFLVEEDKKHERHPIFGNIVDEVAYHEKYPTIYHLRKKLVDSTDKADLRLIYLALA | |
| | HMIKFRGHFLIEGDLNPDNSDVDKLFIQLVQTYNQLFEENPINASGVDAKAILSARLSKSRRLENLIA | |
| | QLPGEKKNGLFGNLIALSLGLTPNFKSNFDLAEDAKLQLSKDTYDDDLDNLLAQIGDQYADLFLAAKN | |
| | LSDAILLSDILRVNTEITKAPLSASMIKRYDEHHQDLTLLKALVRQQLPEKYKEIFFDQSKNGYAGYI | |
| | DGGASQEEFYKFIKPILEKMDGTEELLVKLNREDLLRKQRTFDNGSIPHQIHLGELHAILRRQEDFYP | |
| | FLKDNREKIEKILTFRIPYYVGPLARGNSRFAWMTRKSEETITPWNFEEVVDKGASAQSFIERMTNFD | |
| | KNLPNEKVLPKHSLLYEYFTVYNELTKVKYVTEGMRKPAFLSGEQKKAIVDLLFKTNRKVTVKQLKED | |
| | YFKKIECFDSVEISGVEDRFNASLGTYHDLLKIIKDKDFLDNEENEDILEDIVLTLTLFEDREMIEER | |
| | LKTYAHLFDDKVMKQLKRRRYTGWGRLSRKLINGIRDKQSGKTILDFLKSDGFANRNFMQLIHDDSLT | |
| | FKEDIQKAQVSGQGDSLHEHIANLAGSPAIKKGILQTVKVVDELVKVMGRHKPENIVIEMARENQTTQ | |
| | KGQKNSRERMKRIEEGIKELGSQILKEHPVENTQLQNEKLYLYYLQNGRDMYVDQELDINRLSDYDVD | |
| | HIVPQSFLKDDSIDNKVLTRSDKNRGKSDNVPSEEVVKKMKNYWRQLLNAKLITQRKFDNLTKAERGG | |
| | LSELDKAGFIKRQLVETRQITKHVAQILDSRMNTKYDENDKLIREVKVITLKSKLVSDFRKDFQFYKV | |
| | REINNYHHAHDAYLNAVVGTALIKKYPKLESEFVYGDYKVYDVRKMIAKSEQEIGKATAKYFFYSNIM | |
| | NFFKTEITLANGEIRKRPLIETNGETGEIVWDKGRDFATVRKVLSMPQVNIVKKTEVQTGGFSKESIL | |
| | PKRNSDKLIARKKDWDPKKYGGFDSPTVAYSVLVVAKVEKGKSKKLKSVKELLGITIMERSSFEKNPI | |
| | DFLEAKGYKEVKKDLIIKLPKYSLFELENGRKRMLASAGELQKGNELALPSKYVNFLYLASHYEKLKG | |
| | SPEDNEQKQLFVEQHKHYLDEIIEQISEFSKRVILADANLDKVLSAYNKHRDKPIREQAENIIHLFTL | |
| | TNLGAPAAFKYFDTTIDRKRYTSTKEVLDATLIHQSITGLYETRIDLSQLGGDSGGSGGSGGSTNLSD | |
| | IIEKETGKQLVIQESILMLPEEVEEVIGNKPESDILVHTAYDESTDENVMLLTSDAPEYKPWALVIQD | |
| | SNGENKIKMLSGGSGGSGGSTNLSDIIEKETGKQLVIQESILMLPEEVEEVIGNKPESDILVHTAYDE | |
| | STDENVMLLTSDAPEYKPWALVIQDSNGENKIKMLSGGSKRTADGSEFEPKKKRKV | |
| APOBEC3B | MKRTADGSEFESPKKKRKVNPQIRNPMERMYRDTFYDNFENEPILYGRSYTWLCYEVKIKRGRSNLLW | 231 |
| (A3B)-BE4 | DTGVFRGQVYFKPQYHAEMCFLSWFCGNQLPAYKCFQITWFVSWTPCPDCVAKLAEFLSEHPNVTLTI | |
| | SAARLYYYWERDYRRALCRLSQAGARVTIMDYEEFAYCWENFVYNEGQQFMPWYKFDENYAFLHRTLK | |
| | EILRYLMDPDTFTFNFNNDPLVLRRRQTYLCYEVERLDNGTWVLMDQHMGFLCNEAKNLLCGFYGRHA | |
| | ELRFLDLVPSLQLDPAQIYRVTWFISWSPCFSWGCAGEVRAFLQENTHVRLRIFAARIYDYDPLYKEA | |
| | LQMLRDAGAQVSIMTYDEFEYCWDTFVYRQGCPFQPWDGLEEHSQALSGRLRAILQNQGNSGGSSGGS | |
| | SGSETPGTSESATPESSGGSSGGSDKKYSIGLAIGTNSVGWAVITDEYKVPSKKFKVLGNTDRHSIKK | |
| | NLIGALLFDSGETAEATRLKRTARRRYTRRKNRICYLQEIFSNEMAKVDDSFFHRLEESFLVEEDKKH | |
| | ERHPIFGNIVDEVAYHEKYPTIYHLRKKLVDSTDKADLRLIYLALAHMIKFRGHFLIEGDLNPDNSDV | |
| | DKLFIQLVQTYNQLFEENPINASGVDAKAILSARLSKSRRLENLIAQLPGEKKNGLFGNLIALSLGLT | |
| | PNFKSNFDLAEDAKLQLSKDTYDDDLDNLLAQIGDQYADLFLAAKNLSDAILLSDILRVNTEITKAPL | |
| | SASMIKRYDEHHQDLTLLKALVRQQLPEKYKEIFFDQSKNGYAGYIDGGASQEEFYKFIKPILEKMDG | |
| | TEELLVKLNREDLLRKQRTFDNGSIPHQIHLGELHAILRRQEDFYPFLKDNREKIEKILTFRIPYYVG | |
| | PLARGNSRFAWMTRKSEETITPWNFEEVVDKGASAQSFIERMTNFDKNLPNEKVLPKHSLLYEYFTVY | |
| | NELTKVKYVTEGMRKPAFLSGEQKKAIVDLLFKTNRKVTVKQLKEDYFKKIECFDSVEISGVEDRFNA | |
| | SLGTYHDLLKIIKDKDFLDNEENEDILEDIVLTLTLFEDREMIEERLKTYAHLFDDKVMKQLKRRRYT | |
| | GWGRLSRKLINGIRDKQSGKTILDFLKSDGFANRNFMQLIHDDSLTFKEDIQKAQVSGQGDSLHEHIA | |
| | NLAGSPAIKKGILQTVKVVDELVKVMGRHKPENIVIEMARENQTTQKGQKNSRERMKRIEEGIKELGS | |
| | QILKEHPVENTQLQNEKLYLYYLQNGRDMYVDQELDINRLSDYDVDHIVPQSFLKDDSIDNKVLTRSD | |
| | KNRGKSDNVPSEEVVKKMKNYWRQLLNAKLITQRKFDNLTKAERGGLSELDKAGFIKRQLVETRQITK | |
| | HVAQILDSRMNTKYDENDKLIREVKVITLKSKLVSDFRKDFQFYKVREINNYHHAHDAYLNAVVGTAL | |
| | IKKYPKLESEFVYGDYKVYDVRKMIAKSEQEIGKATAKYFFYSNIMNFFKTEITLANGEIRKRPLIET | |
| | NGETGEIVWDKGRDFATVRKVLSMPQVNIVKKTEVQTGGFSKESILPKRNSDKLIARKKDWDPKKYGG | |
| | FDSPTVAYSVLVVAKVEKGKSKKLKSVKELLGITIMERSSFEKNPIDFLEAKGYKEVKKDLIIKLPKY | |
| | SLFELENGRKRMLASAGELQKGNELALPSKYVNFLYLASHYEKLKGSPEDNEQKQLFVEQHKHYLDEI | |
| | IEQISEFSKRVILADANLDKVLSAYNKHRDKPIREQAENIIHLFTLTNLGAPAAFKYFDTTIDRKRYT | |
| | STKEVLDATLIHQSITGLYETRIDLSQLGGDSGGSGGSGGSTNLSDIIEKETGKQLVIQESILMLPEE | |
| | VEEVIGNKPESDILVHTAYDESTDENVMLLTSDAPEYKPWALVIQDSNGENKIKMLSGGSGGSGGSTN | |
| | LSDIIEKETGKQLVIQESILMLPEEVEEVIGNKPESDILVHTAYDESTDENVMLLTSDAPEYKPWALV | |
| | IQDSNGENKIKMLSGGSKRTADGSEFEPKKKRKV | |
| APOBEC3G | MKRTADGSEFESPKKKRKVKPHFRNTVERMYRDTFSYNFYNRPILSRRNTVWLCYEVKTKGPSRPPLD | 232 |
| (A3G)-BE4 | AKIFRGQVYSELKYHPEMRFFHWFSKWRKLHRDQEYEVTWYISWSPCTKCTRDMATFLAEDPKVTLTI | |
| | FVARLYYFWDPDYQEALRSLCQKRDGPRATMKIMNYDEFQHCWSKFVYSQRELFEPWNNLPKYYILLH | |
| | IMLGEILRHSMDPPTFTFNFNNEPWVRGRHETYLCYEVERMHNDTWVLLNQRRGFLCNQAPHKHGFLE | |
| | GRHAELCFLDVIPFWKLDLDQDYRVTCFTSWSPCFSCAQEMAKFISKNKHVSLCIFTARIYDDQGRCQ | |
| | EGLRTLAEAGAKISIMTYSEFKHCWDTFVDHQGCPFQPWDGLDEHSQDLSGRLRAILQNQENSGGSSG | |
| | GSSGSETPGTSESATPESSGGSSGGSDKKYSIGLAIGTNSVGWAVITDEYKVPSKKFKVLGNTDRHSI | |
| | KKNLIGALLFDSGETAEATRLKRTARRRYTRRKNRICYLQEIFSNEMAKVDDSFFHRLEESFLVEEDK | |
| | KHERHPIFGNIVDEVAYHEKYPTIYHLRKKLVDSTDKADLRLIYLALAHMIKFRGHFLIEGDLNPDNS | |
| | DVDKLFIQLVQTYNQLFEENPINASGVDAKAILSARLSKSRRLENLIAQLPGEKKNGLFGNLIALSLG | |
| | LTPNFKSNFDLAEDAKLQLSKDTYDDDLDNLLAQIGDQYADLFLAAKNLSDAILLSDILRVNTEITKA | |
| | PLSASMIKRYDEHHQDLTLLKALVRQQLPEKYKEIFFDQSKNGYAGYIDGGASQEEFYKFIKPILEKM | |
| | DGTEELLVKLNREDLLRKQRTFDNGSIPHQIHLGELHAILRRQEDFYPFLKDNREKIEKILTFRIPYY | |
| | VGPLARGNSRFAWMTRKSEETITPWNFEEVVDKGASAQSFIERMTNFDKNLPNEKVLPKHSLLYEYFT | |
| | VYNELTKVKYVTEGMRKPAFLSGEQKKAIVDLLFKTNRKVTVKQLKEDYFKKIECFDSVEISGVEDRF | |
| | NASLGTYHDLLKIIKDKDFLDNEENEDILEDIVLTLTLFEDREMIEERLKTYAHLFDDKVMKQLKRRR | |
| | YTGWGRLSRKLINGIRDKQSGKTILDFLKSDGFANRNFMQLIHDDSLTFKEDIQKAQVSGQGDSLHEH | |
| | IANLAGSPAIKKGILQTVKVVDELVKVMGRHKPENIVIEMARENQTTQKGQKNSRERMKRIEEGIKEL | |
| | GSQILKEHPVENTQLQNEKLYLYYLQNGRDMYVDQELDINRLSDYDVDHIVPQSFLKDDSIDNKVLTR | |
| | SDKNRGKSDNVPSEEVVKKMKNYWRQLLNAKLITQRKFDNLTKAERGGLSELDKAGFIKRQLVETRQI | |
| | TKHVAQILDSRMNTKYDENDKLIREVKVITLKSKLVSDFRKDFQFYKVREINNYHHAHDAYLNAVVGT | |
| | ALIKKYPKLESEFVYGDYKVYDVRKMIAKSEQEIGKATAKYFFYSNIMNFFKTEITLANGEIRKRPLI | |
| | ETNGETGEIVWDKGRDFATVRKVLSMPQVNIVKKTEVQTGGFSKESILPKRNSDKLIARKKDWDPKKY | |
| | GGFDSPTVAYSVLVVAKVEKGKSKKLKSVKELLGITIMERSSFEKNPIDFLEAKGYKEVKKDLIIKLP | |
| | KYSLFELENGRKRMLASAGELQKGNELALPSKYVNFLYLASHYEKLKGSPEDNEQKQLFVEQHKHYLD | |
| | EIIEQISEFSKRVILADANLDKVLSAYNKHRDKPIREQAENIIHLFTLTNLGAPAAFKYFDTTIDRKR | |
| | YTSTKEVLDATLIHQSITGLYETRIDLSQLGGDSGGSGGSGGSTNLSDIIEKETGKQLVIQESILMLP | |
| | EEVEEVIGNKPESDILVHTAYDESTDENVMLLTSDAPEYKPWALVIQDSNGENKIKMLSGGSGGSGGS | |
| | TNLSDIIEKETGKQLVIQESILMLPEEVEEVIGNKPESDILVHTAYDESTDENVMLLTSDAPEYKPWA | |
| | LVIQDSNGENKIKMLSGGSKRTADGSEFEPKKKRKV | |
| AID-BE4 | MKRTADGSEFESPKKKRKVDSLLMNRRKFLYQFKNVRWAKGRRETYLCYVVKRRDSATSFSLDFGYLR | 233 |
| | NKNGCHVELLFLRYISDWDLDPGRCYRVTWFTSWSPCYDCARHVADFLRGNPNLSLRIFTARLYFCED | |
| | RKAEPEGLRRLHRAGVQIAIMTFKDYFYCWNTFVENHERTFKAWEGLHENSVRLSRQLRRILLPLYEV | |
| | DDLRDAFRTLGLSGGSSGGSSGSETPGTSESATPESSGGSSGGSDKKYSIGLAIGTNSVGWAVITDEY | |
| | KVPSKKFKVLGNTDRHSIKKNLIGALLFDSGETAEATRLKRTARRRYTRRKNRICYLQEIFSNEMAKV | |
| | DDSFFHRLEESFLVEEDKKHERHPIFGNIVDEVAYHEKYPTIYHLRKKLVDSTDKADLRLIYLALAHM | |
| | IKFRGHFLIEGDLNPDNSDVDKLFIQLVQTYNQLFEENPINASGVDAKAILSARLSKSRRLENLIAQL | |
| | PGEKKNGLFGNLIALSLGLTPNFKSNFDLAEDAKLQLSKDTYDDDLDNLLAQIGDQYADLFLAAKNLS | |
| | DAILLSDILRVNTEITKAPLSASMIKRYDEHHQDLTLLKALVRQQLPEKYKEIFFDQSKNGYAGYIDG | |
| | GASQEEFYKFIKPILEKMDGTEELLVKLNREDLLRKQRTFDNGSIPHQIHLGELHAILRRQEDFYPFL | |
| | KDNREKIEKILTFRIPYYVGPLARGNSRFAWMTRKSEETITPWNFEEVVDKGASAQSFIERMTNFDKN | |
| | LPNEKVLPKHSLLYEYFTVYNELTKVKYVTEGMRKPAFLSGEQKKAIVDLLFKTNRKVTVKQLKEDYF | |
| | KKIECFDSVEISGVEDRFNASLGTYHDLLKIIKDKDFLDNEENEDILEDIVLTLTLFEDREMIEERLK | |
| | TYAHLFDDKVMKQLKRRRYTGWGRLSRKLINGIRDKQSGKTILDFLKSDGFANRNFMQLIHDDSLTFK | |
| | EDIQKAQVSGQGDSLHEHIANLAGSPAIKKGILQTVKVVDELVKVMGRHKPENIVIEMARENQTTQKG | |
| | QKNSRERMKRIEEGIKELGSQILKEHPVENTQLQNEKLYLYYLQNGRDMYVDQELDINRLSDYDVDHI | |
| | VPQSFLKDDSIDNKVLTRSDKNRGKSDNVPSEEVVKKMKNYWRQLLNAKLITQRKFDNLTKAERGGLS | |
| | ELDKAGFIKRQLVETRQITKHVAQILDSRMNTKYDENDKLIREVKVITLKSKLVSDFRKDFQFYKVRE | |
| | INNYHHAHDAYLNAVVGTALIKKYPKLESEFVYGDYKVYDVRKMIAKSEQEIGKATAKYFFYSNIMNF | |
| | FKTEITLANGEIRKRPLIETNGETGEIVWDKGRDFATVRKVLSMPQVNIVKKTEVQTGGFSKESILPK | |
| | RNSDKLIARKKDWDPKKYGGFDSPTVAYSVLVVAKVEKGKSKKLKSVKELLGITIMERSSFEKNPIDF | |
| | LEAKGYKEVKKDLIIKLPKYSLFELENGRKRMLASAGELQKGNELALPSKYVNFLYLASHYEKLKGSP | |
| | EDNEQKQLFVEQHKHYLDEIIEQISEFSKRVILADANLDKVLSAYNKHRDKPIREQAENIIHLFTLTN | |
| | LGAPAAFKYFDTTIDRKRYTSTKEVLDATLIHQSITGLYETRIDLSQLGGDSGGSGGSGGSTNLSDII | |
| | EKETGKQLVIQESILMLPEEVEEVIGNKPESDILVHTAYDESTDENVMLLTSDAPEYKPWALVIQDSN | |
| | GENKIKMLSGGSGGSGGSTNLSDIIEKETGKQLVIQESILMLPEEVEEVIGNKPESDILVHTAYDEST | |
| | DENVMLLTSDAPEYKPWALVIQDSNGENKIKMLSGGSKRTADGSEFEPKKKRKV | |
| CDA-BE4 | MKRTADGSEFESPKKKRKVTDAEYVRIHEKLDIYTFKKQFFNNKKSVSHRCYVLFELKRRGERRACFW | 234 |
| | GYAVNKPQSGTERGIHAEIFSIRKVEEYLRDNPGQFTINWYSSWSPCADCAEKILEWYNQELRGNGHT | |
| | LKIWACKLYYEKNARNQIGLWNLRDNGVGLNVMVSEHYQCCRKIFIQSSHNQLNENRWLEKTLKRAEK | |
| | RRSELSIMIQVKILHTTKSPAVSGGSSGGSSGSETPGTSESATPESSGGSSGGSDKKYSIGLAIGTNS | |
| | VGWAVITDEYKVPSKKFKVLGNTDRHSIKKNLIGALLFDSGETAEATRLKRTARRRYTRRKNRICYLQ | |
| | EIFSNEMAKVDDSFFHRLEESFLVEEDKKHERHPIFGNIVDEVAYHEKYPTIYHLRKKLVDSTDKADL | |
| | RLIYLALAHMIKFRGHFLIEGDLNPDNSDVDKLFIQLVQTYNQLFEENPINASGVDAKAILSARLSKS | |
| | RRLENLIAQLPGEKKNGLFGNLIALSLGLTPNFKSNFDLAEDAKLQLSKDTYDDDLDNLLAQIGDQYA | |
| | DLFLAAKNLSDAILLSDILRVNTEITKAPLSASMIKRYDEHHQDLTLLKALVRQQLPEKYKEIFFDQS | |
| | KNGYAGYIDGGASQEEFYKFIKPILEKMDGTEELLVKLNREDLLRKQRTFDNGSIPHQIHLGELHAIL | |
| | RRQEDFYPFLKDNREKIEKILTFRIPYYVGPLARGNSRFAWMTRKSEETITPWNFEEVVDKGASAQSF | |
| | IERMTNFDKNLPNEKVLPKHSLLYEYFTVYNELTKVKYVTEGMRKPAFLSGEQKKAIVDLLFKTNRKV | |
| | TVKQLKEDYFKKIECFDSVEISGVEDRFNASLGTYHDLLKIIKDKDFLDNEENEDILEDIVLTLTLFE | |
| | DREMIEERLKTYAHLFDDKVMKQLKRRRYTGWGRLSRKLINGIRDKQSGKTILDFLKSDGFANRNFMQ | |
| | LIHDDSLTFKEDIQKAQVSGQGDSLHEHIANLAGSPAIKKGILQTVKVVDELVKVMGRHKPENIVIEM | |
| | ARENQTTQKGQKNSRERMKRIEEGIKELGSQILKEHPVENTQLQNEKLYLYYLQNGRDMYVDQELDIN | |
| | RLSDYDVDHIVPQSFLKDDSIDNKVLTRSDKNRGKSDNVPSEEVVKKMKNYWRQLLNAKLITQRKFDN | |
| | LTKAERGGLSELDKAGFIKRQLVETRQITKHVAQILDSRMNTKYDENDKLIREVKVITLKSKLVSDFR | |
| | KDFQFYKVREINNYHHAHDAYLNAVVGTALIKKYPKLESEFVYGDYKVYDVRKMIAKSEQEIGKATAK | |
| | YFFYSNIMNFFKTEITLANGEIRKRPLIETNGETGEIVWDKGRDFATVRKVLSMPQVNIVKKTEVQTG | |
| | GFSKESILPKRNSDKLIARKKDWDPKKYGGFDSPTVAYSVLVVAKVEKGKSKKLKSVKELLGITIMER | |
| | SSFEKNPIDFLEAKGYKEVKKDLIIKLPKYSLFELENGRKRMLASAGELQKGNELALPSKYVNFLYLA | |
| | SHYEKLKGSPEDNEQKQLFVEQHKHYLDEIIEQISEFSKRVILADANLDKVLSAYNKHRDKPIREQAE | |
| | NIIHLFTLTNLGAPAAFKYFDTTIDRKRYTSTKEVLDATLIHQSITGLYETRIDLSQLGGDSGGSGGS | |
| | GGSTNLSDIIEKETGKQLVIQESILMLPEEVEEVIGNKPESDILVHTAYDESTDENVMLLTSDAPEYK | |
| | PWALVIQDSNGENKIKMLSGGSGGSGGSTNLSDIIEKETGKQLVIQESILMLPEEVEEVIGNKPESDI | |
| | LVHTAYDESTDENVMLLTSDAPEYKPWALVIQDSNGENKIKMLSGGSKRTADGSEFEPKKKRKV | |
| FERNY-BE4 | MKRTADGSEFESPKKKRKVFERNYDPRELRKETYLLYEIKWGKSGKLWRHWCQNNRTQHAEVYFLENI | 235 |
| | FNARRFNPSTHCSITWYLSWSPCAECSQKIVDFLKEHPNVNLEIYVARLYYHEDERNRQGLRDLVNSG | |
| | VTIRIMDLPDYNYCWKTFVSDQGGDEDYWPGHFAPWIKQYSLKLSGGSSGGSSGSETPGTSESATPES | |
| | SGGSSGGSDKKYSIGLAIGTNSVGWAVITDEYKVPSKKFKVLGNTDRHSIKKNLIGALLFDSGETAEA | |
| | TRLKRTARRRYTRRKNRICYLQEIFSNEMAKVDDSFFHRLEESFLVEEDKKHERHPIFGNIVDEVAYH | |
| | EKYPTIYHLRKKLVDSTDKADLRLIYLALAHMIKFRGHFLIEGDLNPDNSDVDKLFIQLVQTYNQLFE | |
| | ENPINASGVDAKAILSARLSKSRRLENLIAQLPGEKKNGLFGNLIALSLGLTPNFKSNFDLAEDAKLQ | |
| | LSKDTYDDDLDNLLAQIGDQYADLFLAAKNLSDAILLSDILRVNTEITKAPLSASMIKRYDEHHQDLT | |
| | LLKALVRQQLPEKYKEIFFDQSKNGYAGYIDGGASQEEFYKFIKPILEKMDGTEELLVKLNREDLLRK | |
| | QRTFDNGSIPHQIHLGELHAILRRQEDFYPFLKDNREKIEKILTFRIPYYVGPLARGNSRFAWMTRKS | |
| | EETITPWNFEEVVDKGASAQSFIERMTNFDKNLPNEKVLPKHSLLYEYFTVYNELTKVKYVTEGMRKP | |
| | AFLSGEQKKAIVDLLFKTNRKVTVKQLKEDYFKKIECFDSVEISGVEDRFNASLGTYHDLLKIIKDKD | |
| | FLDNEENEDILEDIVLTLTLFEDREMIEERLKTYAHLFDDKVMKQLKRRRYTGWGRLSRKLINGIRDK | |
| | QSGKTILDFLKSDGFANRNFMQLIHDDSLTFKEDIQKAQVSGQGDSLHEHIANLAGSPAIKKGILQTV | |
| | KVVDELVKVMGRHKPENIVIEMARENQTTQKGQKNSRERMKRIEEGIKELGSQILKEHPVENTQLQNE | |
| | KLYLYYLQNGRDMYVDQELDINRLSDYDVDHIVPQSFLKDDSIDNKVLTRSDKNRGKSDNVPSEEVVK | |
| | KMKNYWRQLLNAKLITQRKFDNLTKAERGGLSELDKAGFIKRQLVETRQITKHVAQILDSRMNTKYDE | |
| | NDKLIREVKVITLKSKLVSDFRKDFQFYKVREINNYHHAHDAYLNAVVGTALIKKYPKLESEFVYGDY | |
| | KVYDVRKMIAKSEQEIGKATAKYFFYSNIMNFFKTEITLANGEIRKRPLIETNGETGEIVWDKGRDFA | |
| | TVRKVLSMPQVNIVKKTEVQTGGFSKESILPKRNSDKLIARKKDWDPKKYGGFDSPTVAYSVLVVAKV | |
| | EKGKSKKLKSVKELLGITIMERSSFEKNPIDFLEAKGYKEVKKDLIIKLPKYSLFELENGRKRMLASA | |
| | GELQKGNELALPSKYVNFLYLASHYEKLKGSPEDNEQKQLFVEQHKHYLDEIIEQISEFSKRVILADA | |
| | NLDKVLSAYNKHRDKPIREQAENIIHLFTLTNLGAPAAFKYFDTTIDRKRYTSTKEVLDATLIHQSIT | |
| | GLYETRIDLSQLGGDSGGSGGSGGSTNLSDIIEKETGKQLVIQESILMLPEEVEEVIGNKPESDILVH | |
| | TAYDESTDENVMLLTSDAPEYKPWALVIQDSNGENKIKMLSGGSGGSGGSTNLSDIIEKETGKQLVIQ | |
| | ESILMLPEEVEEVIGNKPESDILVHTAYDESTDENVMLLTSDAPEYKPWALVIQDSNGENKIKMLSGG | |
| | SKRTADGSEFEPKKKRKV | |
| Evolved | MKRTADGSEFESPKKKRKVEASPASGPRHLMDPHIFTSNFNNGIGRHKTYLCYEVERLDNGTSVKMDQ | 236 |
| APOBEC3A | HRGFLHGQAKNLLCGFYGRHAELRFLDLVPSLQLDPAQIYRVTWFISWSPCFSWGCAGEVRAFLQENT | |
| (eA3A)-BE4 | HVRLRIFAARIYDYDPLYKEALQMLRDAGAQVSIMTYDEFKHCWDTFVDHQGCPFQPWDGLDEHSQAL | |
| | SGRLRAILQNQGNSGGSSGGSSGSETPGTSESATPESSGGSSGGSDKKYSIGLAIGTNSVGWAVITDE | |
| | YKVPSKKFKVLGNTDRHSIKKNLIGALLFDSGETAEATRLKRTARRRYTRRKNRICYLQEIFSNEMAK | |
| | VDDSFFHRLEESFLVEEDKKHERHPIFGNIVDEVAYHEKYPTIYHLRKKLVDSTDKADLRLIYLALAH | |
| | MIKFRGHFLIEGDLNPDNSDVDKLFIQLVQTYNQLFEENPINASGVDAKAILSARLSKSRRLENLIAQ | |
| | LPGEKKNGLFGNLIALSLGLTPNFKSNFDLAEDAKLQLSKDTYDDDLDNLLAQIGDQYADLFLAAKNL | |
| | SDAILLSDILRVNTEITKAPLSASMIKRYDEHHQDLTLLKALVRQQLPEKYKEIFFDQSKNGYAGYID | |
| | GGASQEEFYKFIKPILEKMDGTEELLVKLNREDLLRKQRTFDNGSIPHQIHLGELHAILRRQEDFYPF | |
| | LKDNREKIEKILTFRIPYYVGPLARGNSRFAWMTRKSEETITPWNFEEVVDKGASAQSFIERMTNFDK | |
| | NLPNEKVLPKHSLLYEYFTVYNELTKVKYVTEGMRKPAFLSGEQKKAIVDLLFKTNRKVTVKQLKEDY | |
| | FKKIECFDSVEISGVEDRFNASLGTYHDLLKIIKDKDFLDNEENEDILEDIVLTLTLFEDREMIEERL | |
| | KTYAHLFDDKVMKQLKRRRYTGWGRLSRKLINGIRDKQSGKTILDFLKSDGFANRNFMQLIHDDSLTF | |
| | KEDIQKAQVSGQGDSLHEHIANLAGSPAIKKGILQTVKVVDELVKVMGRHKPENIVIEMARENQTTQK | |
| | GQKNSRERMKRIEEGIKELGSQILKEHPVENTQLQNEKLYLYYLQNGRDMYVDQELDINRLSDYDVDH | |
| | IVPQSFLKDDSIDNKVLTRSDKNRGKSDNVPSEEVVKKMKNYWRQLLNAKLITQRKFDNLTKAERGGL | |
| | SELDKAGFIKRQLVETRQITKHVAQILDSRMNTKYDENDKLIREVKVITLKSKLVSDFRKDFQFYKVR | |
| | EINNYHHAHDAYLNAVVGTALIKKYPKLESEFVYGDYKVYDVRKMIAKSEQEIGKATAKYFFYSNIMN | |
| | FFKTEITLANGEIRKRPLIETNGETGEIVWDKGRDFATVRKVLSMPQVNIVKKTEVQTGGFSKESILP | |
| | KRNSDKLIARKKDWDPKKYGGFDSPTVAYSVLVVAKVEKGKSKKLKSVKELLGITIMERSSFEKNPID | |
| | FLEAKGYKEVKKDLIIKLPKYSLFELENGRKRMLASAGELQKGNELALPSKYVNFLYLASHYEKLKGS | |
| | PEDNEQKQLFVEQHKHYLDEIIEQISEFSKRVILADANLDKVLSAYNKHRDKPIREQAENIIHLFTLT | |
| | NLGAPAAFKYFDTTIDRKRYTSTKEVLDATLIHQSITGLYETRIDLSQLGGDSGGSGGSGGSTNLSDI | |
| | IEKETGKQLVIQESILMLPEEVEEVIGNKPESDILVHTAYDESTDENVMLLTSDAPEYKPWALVIQDS | |
| | NGENKIKMLSGGSGGSGGSTNLSDIIEKETGKQLVIQESILMLPEEVEEVIGNKPESDILVHTAYDES | |
| | TDENVMLLTSDAPEYKPWALVIQDSNGENKIKMLSGGSKRTADGSEFEPKKKRKV | |
| AALN-BE4 | MKRTADGSEFESPKKKRKVSSETGPVAVDPTLRRRIEPHEFEVFFDPRELAAETCLLYEINWGGRHSI | 237 |
| | WRHTSQNTNKHVEVNFIEKFTTERYFCPNTRCSITWFLSWSPCGECSRAITEFLSRYPHVTLFIYIAR | |
| | LYHLANPRNRQGLRDLISSGVTIQIMTEQESGYCWRNFVNYSPSNEAHWPRYPHLWVRLYVLELYCII | |
| | LGLPPCLNILRRKQPQLTFFTIALQSCHYQRLPPHILWATGLKSGGSSGGSSGSETPGTSESATPESS | |
| | GGSSGGSDKKYSIGLAIGTNSVGWAVITDEYKVPSKKFKVLGNTDRHSIKKNLIGALLFDSGETAEAT | |
| | RLKRTARRRYTRRKNRICYLQEIFSNEMAKVDDSFFHRLEESFLVEEDKKHERHPIFGNIVDEVAYHE | |
| | KYPTIYHLRKKLVDSTDKADLRLIYLALAHMIKFRGHFLIEGDLNPDNSDVDKLFIQLVQTYNQLFEE | |
| | NPINASGVDAKAILSARLSKSRRLENLIAQLPGEKKNGLFGNLIALSLGLTPNFKSNFDLAEDAKLQL | |
| | SKDTYDDDLDNLLAQIGDQYADLFLAAKNLSDAILLSDILRVNTEITKAPLSASMIKRYDEHHQDLTL | |
| | LKALVRQQLPEKYKEIFFDQSKNGYAGYIDGGASQEEFYKFIKPILEKMDGTEELLVKLNREDLLRKQ | |
| | RTFDNGSIPHQIHLGELHAILRRQEDFYPFLKDNREKIEKILTFRIPYYVGPLARGNSRFAWMTRKSE | |
| | ETITPWNFEEVVDKGASAQSFIERMTNFDKNLPNEKVLPKHSLLYEYFTVYNELTKVKYVTEGMRKPA | |
| | FLSGEQKKAIVDLLFKTNRKVTVKQLKEDYFKKIECFDSVEISGVEDRFNASLGTYHDLLKIIKDKDF | |
| | LDNEENEDILEDIVLTLTLFEDREMIEERLKTYAHLFDDKVMKQLKRRRYTGWGRLSRKLINGIRDKQ | |
| | SGKTILDFLKSDGFANRNFMQLIHDDSLTFKEDIQKAQVSGQGDSLHEHIANLAGSPAIKKGILQTVK | |
| | VVDELVKVMGRHKPENIVIEMARENQTTQKGQKNSRERMKRIEEGIKELGSQILKEHPVENTQLQNEK | |
| | LYLYYLQNGRDMYVDQELDINRLSDYDVDHIVPQSFLKDDSIDNKVLTRSDKNRGKSDNVPSEEVVKK | |
| | MKNYWRQLLNAKLITQRKFDNLTKAERGGLSELDKAGFIKRQLVETRQITKHVAQILDSRMNTKYDEN | |
| | DKLIREVKVITLKSKLVSDFRKDFQFYKVREINNYHHAHDAYLNAVVGTALIKKYPKLESEFVYGDYK | |
| | VYDVRKMIAKSEQEIGKATAKYFFYSNIMNFFKTEITLANGEIRKRPLIETNGETGEIVWDKGRDFAT | |
| | VRKVLSMPQVNIVKKTEVQTGGFSKESILPKRNSDKLIARKKDWDPKKYGGFDSPTVAYSVLVVAKVE | |
| | KGKSKKLKSVKELLGITIMERSSFEKNPIDFLEAKGYKEVKKDLIIKLPKYSLFELENGRKRMLASAG | |
| | ELQKGNELALPSKYVNFLYLASHYEKLKGSPEDNEQKQLFVEQHKHYLDEIIEQISEFSKRVILADAN | |
| | LDKVLSAYNKHRDKPIREQAENIIHLFTLTNLGAPAAFKYFDTTIDRKRYTSTKEVLDATLIHQSITG | |
| | LYETRIDLSQLGGDSGGSGGSGGSTNLSDIIEKETGKQLVIQESILMLPEEVEEVIGNKPESDILVHT | |
| | AYDESTDENVMLLTSDAPEYKPWALVIQDSNGENKIKMLSGGSGGSGGSTNLSDIIEKETGKQLVIQE | |
| | SILMLPEEVEEVIGNKPESDILVHTAYDESTDENVMLLTSDAPEYKPWALVIQDSNGENKIKMLSGGS | |
| | KRTADGSEFEPKKKRKV | |
| BE4max, | MKRTADGSEFESPKKKRKVSSETGPVAVDPTLRRRIEPHEFEVFFDPRELRKETCLLYEINWGGRHSI | 238 |
| modified with | WRHTSQNTNKHVEVNFIEKFTTERYFCPNTRCSITWFLSWSPCGECSRAITEFLSRYPHVTLFIYIAR | |
| SpCas9-NG | LYHHADPRNRQGLRDLISSGVTIQIMTEQESGYCWRNFVNYSPSNEAHWPRYPHLWVRLYVLELYCII | |
| | LGLPPCLNILRRKQPQLTFFTIALQSCHYQRLPPHILWATGLKSGGSSGGSSGSETPGTSESATPESS | |
| | GGSSGGSDKKYSIGLAIGTNSVGWAVITDEYKVPSKKFKVLGNTDRHSIKKNLIGALLFDSGETAEAT | |
| | RLKRTARRRYTRRKNRICYLQEIFSNEMAKVDDSFFHRLEESFLVEEDKKHERHPIFGNIVDEVAYHE | |
| | KYPTIYHLRKKLVDSTDKADLRLIYLALAHMIKFRGHFLIEGDLNPDNSDVDKLFIQLVQTYNQLFEE | |
| | NPINASGVDAKAILSARLSKSRRLENLIAQLPGEKKNGLFGNLIALSLGLTPNFKSNFDLAEDAKLQL | |
| | SKDTYDDDLDNLLAQIGDQYADLFLAAKNLSDAILLSDILRVNTEITKAPLSASMIKRYDEHHQDLTL | |
| | LKALVRQQLPEKYKEIFFDQSKNGYAGYIDGGASQEEFYKFIKPILEKMDGTEELLVKLNREDLLRKQ | |
| | RTFDNGSIPHQIHLGELHAILRRQEDFYPFLKDNREKIEKILTFRIPYYVGPLARGNSRFAWMTRKSE | |
| | ETITPWNFEEVVDKGASAQSFIERMTNFDKNLPNEKVLPKHSLLYEYFTVYNELTKVKYVTEGMRKPA | |
| | FLSGEQKKAIVDLLFKTNRKVTVKQLKEDYFKKIECFDSVEISGVEDRFNASLGTYHDLLKIIKDKDF | |
| | LDNEENEDILEDIVLTLTLFEDREMIEERLKTYAHLFDDKVMKQLKRRRYTGWGRLSRKLINGIRDKQ | |
| | SGKTILDFLKSDGFANRNFMQLIHDDSLTFKEDIQKAQVSGQGDSLHEHIANLAGSPAIKKGILQTVK | |
| | VVDELVKVMGRHKPENIVIEMARENQTTQKGQKNSRERMKRIEEGIKELGSQILKEHPVENTQLQNEK | |
| | LYLYYLQNGRDMYVDQELDINRLSDYDVDHIVPQSFLKDDSIDNKVLTRSDKNRGKSDNVPSEEVVKK | |
| | MKNYWRQLLNAKLITQRKFDNLTKAERGGLSELDKAGFIKRQLVETRQITKHVAQILDSRMNTKYDEN | |
| | DKLIREVKVITLKSKLVSDFRKDFQFYKVREINNYHHAHDAYLNAVVGTALIKKYPKLESEFVYGDYK | |
| | VYDVRKMIAKSEQEIGKATAKYFFYSNIMNFFKTEITLANGEIRKRPLIETNGETGEIVWDKGRDFAT | |
| | VRKVLSMPQVNIVKKTEVQTGGFSKESIRPKRNSDKLIARKKDWDPKKYGGFVSPTVAYSVLVVAKVE | |
| | KGKSKKLKSVKELLGITIMERSSFEKNPIDFLEAKGYKEVKKDLIIKLPKYSLFELENGRKRMLASAR | |
| | FLQKGNELALPSKYVNFLYLASHYEKLKGSPEDNEQKQLFVEQHKHYLDEIIEQISEFSKRVILADAN | |
| | LDKVLSAYNKHRDKPIREQAENIIHLFTLTNLGAPRAFKYFDTTIDRKVYRSTKEVLDATLIHQSITG | |
| | LYETRIDLSQLGGDSGGSGGSGGSTNLSDIIEKETGKQLVIQESILMLPEEVEEVIGNKPESDILVHT | |
| | AYDESTDENVMLLTSDAPEYKPWALVIQDSNGENKIKMLSGGSGGSGGSTNLSDIIEKETGKQLVIQE | |
| | SILMLPEEVEEVIGNKPESDILVHTAYDESTDENVMLLTSDAPEYKPWALVIQDSNGENKIKMLSGGS | |
| | KRTADGSEFEPKKKRKV | |
| YEl-SpCas9-NG | MKRTADGSEFESPKKKRKVSSETGPVAVDPTLRRRIEPHEFEVFFDPRELRKETCLLYEINWGGRHSI | 239 |
| base editor | WRHTSQNTNKHVEVNFIEKFTTERYFCPNTRCSITWFLSYSPCGECSRAITEFLSRYPHVTLFIYIAR | |
| (YEl-NG) | LYHHADPENRQGLRDLISSGVTIQIMTEQESGYCWRNFVNYSPSNEAHWPRYPHLWVRLYVLELYCII | |
| | LGLPPCLNILRRKQPQLTFFTIALQSCHYQRLPPHILWATGLKSGGSSGGSSGSETPGTSESATPESS | |
| | GGSSGGSDKKYSIGLAIGTNSVGWAVITDEYKVPSKKFKVLGNTDRHSIKKNLIGALLFDSGETAEAT | |
| | RLKRTARRRYTRRKNRICYLQEIFSNEMAKVDDSFFHRLEESFLVEEDKKHERHPIFGNIVDEVAYHE | |
| | KYPTIYHLRKKLVDSTDKADLRLIYLALAHMIKFRGHFLIEGDLNPDNSDVDKLFIQLVQTYNQLFEE | |
| | NPINASGVDAKAILSARLSKSRRLENLIAQLPGEKKNGLFGNLIALSLGLTPNFKSNFDLAEDAKLQL | |
| | SKDTYDDDLDNLLAQIGDQYADLFLAAKNLSDAILLSDILRVNTEITKAPLSASMIKRYDEHHQDLTL | |
| | LKALVRQQLPEKYKEIFFDQSKNGYAGYIDGGASQEEFYKFIKPILEKMDGTEELLVKLNREDLLRKQ | |
| | RTFDNGSIPHQIHLGELHAILRRQEDFYPFLKDNREKIEKILTFRIPYYVGPLARGNSRFAWMTRKSE | |
| | ETITPWNFEEVVDKGASAQSFIERMTNFDKNLPNEKVLPKHSLLYEYFTVYNELTKVKYVTEGMRKPA | |
| | FLSGEQKKAIVDLLFKTNRKVTVKQLKEDYFKKIECFDSVEISGVEDRFNASLGTYHDLLKIIKDKDF | |
| | LDNEENEDILEDIVLTLTLFEDREMIEERLKTYAHLFDDKVMKQLKRRRYTGWGRLSRKLINGIRDKQ | |
| | SGKTILDFLKSDGFANRNFMQLIHDDSLTFKEDIQKAQVSGQGDSLHEHIANLAGSPAIKKGILQTVK | |
| | VVDELVKVMGRHKPENIVIEMARENQTTQKGQKNSRERMKRIEEGIKELGSQILKEHPVENTQLQNEK | |
| | LYLYYLQNGRDMYVDQELDINRLSDYDVDHIVPQSFLKDDSIDNKVLTRSDKNRGKSDNVPSEEVVKK | |
| | MKNYWRQLLNAKLITQRKFDNLTKAERGGLSELDKAGFIKRQLVETRQITKHVAQILDSRMNTKYDEN | |
| | DKLIREVKVITLKSKLVSDFRKDFQFYKVREINNYHHAHDAYLNAVVGTALIKKYPKLESEFVYGDYK | |
| | VYDVRKMIAKSEQEIGKATAKYFFYSNIMNFFKTEITLANGEIRKRPLIETNGETGEIVWDKGRDFAT | |
| | VRKVLSMPQVNIVKKTEVQTGGFSKESIRPKRNSDKLIARKKDWDPKKYGGFVSPTVAYSVLVVAKVE | |
| | KGKSKKLKSVKELLGITIMERSSFEKNPIDFLEAKGYKEVKKDLIIKLPKYSLFELENGRKRMLASAR | |
| | FLQKGNELALPSKYVNFLYLASHYEKLKGSPEDNEQKQLFVEQHKHYLDEIIEQISEFSKRVILADAN | |
| | LDKVLSAYNKHRDKPIREQAENIIHLFTLTNLGAPRAFKYFDTTIDRKVYRSTKEVLDATLIHQSITG | |
| | LYETRIDLSQLGGDSGGSGGSGGSTNLSDIIEKETGKQLVIQESILMLPEEVEEVIGNKPESDILVHT | |
| | AYDESTDENVMLLTSDAPEYKPWALVIQDSNGENKIKMLSGGSGGSGGSTNLSDIIEKETGKQLVIQE | |
| | SILMLPEEVEEVIGNKPESDILVHTAYDESTDENVMLLTSDAPEYKPWALVIQDSNGENKIKMLSGGS | |
| | KRTADGSEFEPKKKRKV | |
| YE2-SpCas9-NG | MKRTADGSEFESPKKKRKVSSETGPVAVDPTLRRRIEPHEFEVFFDPRELRKETCLLYEINWGGRHSI | 240 |
| base editor | WRHTSQNTNKHVEVNFIEKFTTERYFCPNTRCSITWFLSYSPCGECSRAITEFLSRYPHVTLFIYIAR | |
| | LYHHADPRNRQGLEDLISSGVTIQIMTEQESGYCWRNFVNYSPSNEAHWPRYPHLWVRLYVLELYCII | |
| | LGLPPCLNILRRKQPQLTFFTIALQSCHYQRLPPHILWATGLKSGGSSGGSSGSETPGTSESATPESS | |
| | GGSSGGSDKKYSIGLAIGTNSVGWAVITDEYKVPSKKFKVLGNTDRHSIKKNLIGALLFDSGETAEAT | |
| | RLKRTARRRYTRRKNRICYLQEIFSNEMAKVDDSFFHRLEESFLVEEDKKHERHPIFGNIVDEVAYHE | |
| | KYPTIYHLRKKLVDSTDKADLRLIYLALAHMIKFRGHFLIEGDLNPDNSDVDKLFIQLVQTYNQLFEE | |
| | NPINASGVDAKAILSARLSKSRRLENLIAQLPGEKKNGLFGNLIALSLGLTPNFKSNFDLAEDAKLQL | |
| | SKDTYDDDLDNLLAQIGDQYADLFLAAKNLSDAILLSDILRVNTEITKAPLSASMIKRYDEHHQDLTL | |
| | LKALVRQQLPEKYKEIFFDQSKNGYAGYIDGGASQEEFYKFIKPILEKMDGTEELLVKLNREDLLRKQ | |
| | RTFDNGSIPHQIHLGELHAILRRQEDFYPFLKDNREKIEKILTFRIPYYVGPLARGNSRFAWMTRKSE | |
| | ETITPWNFEEVVDKGASAQSFIERMTNFDKNLPNEKVLPKHSLLYEYFTVYNELTKVKYVTEGMRKPA | |
| | FLSGEQKKAIVDLLFKTNRKVTVKQLKEDYFKKIECFDSVEISGVEDRFNASLGTYHDLLKIIKDKDF | |
| | LDNEENEDILEDIVLTLTLFEDREMIEERLKTYAHLFDDKVMKQLKRRRYTGWGRLSRKLINGIRDKQ | |
| | SGKTILDFLKSDGFANRNFMQLIHDDSLTFKEDIQKAQVSGQGDSLHEHIANLAGSPAIKKGILQTVK | |
| | VVDELVKVMGRHKPENIVIEMARENQTTQKGQKNSRERMKRIEEGIKELGSQILKEHPVENTQLQNEK | |
| | LYLYYLQNGRDMYVDQELDINRLSDYDVDHIVPQSFLKDDSIDNKVLTRSDKNRGKSDNVPSEEVVKK | |
| | MKNYWRQLLNAKLITQRKFDNLTKAERGGLSELDKAGFIKRQLVETRQITKHVAQILDSRMNTKYDEN | |
| | DKLIREVKVITLKSKLVSDFRKDFQFYKVREINNYHHAHDAYLNAVVGTALIKKYPKLESEFVYGDYK | |
| | VYDVRKMIAKSEQEIGKATAKYFFYSNIMNFFKTEITLANGEIRKRPLIETNGETGEIVWDKGRDFAT | |
| | VRKVLSMPQVNIVKKTEVQTGGFSKESIRPKRNSDKLIARKKDWDPKKYGGFVSPTVAYSVLVVAKVE | |
| | KGKSKKLKSVKELLGITIMERSSFEKNPIDFLEAKGYKEVKKDLIIKLPKYSLFELENGRKRMLASAR | |
| | FLQKGNELALPSKYVNFLYLASHYEKLKGSPEDNEQKQLFVEQHKHYLDEIIEQISEFSKRVILADAN | |
| | LDKVLSAYNKHRDKPIREQAENIIHLFTLTNLGAPRAFKYFDTTIDRKVYRSTKEVLDATLIHQSITG | |
| | LYETRIDLSQLGGDSGGSGGSGGSTNLSDIIEKETGKQLVIQESILMLPEEVEEVIGNKPESDILVHT | |
| | AYDESTDENVMLLTSDAPEYKPWALVIQDSNGENKIKMLSGGSGGSGGSTNLSDIIEKETGKQLVIQE | |
| | SILMLPEEVEEVIGNKPESDILVHTAYDESTDENVMLLTSDAPEYKPWALVIQDSNGENKIKMLSGGS | |
| | KRTADGSEFEPKKKRKV | |
| YEE-SpCas9-NG | MKRTADGSEFESPKKKRKVSSETGPVAVDPTLRRRIEPHEFEVFFDPRELRKETCLLYEINWGGRHSI | 241 |
| base editor | WRHTSQNTNKHVEVNFIEKFTTERYFCPNTRCSITWFLSYSPCGECSRAITEFLSRYPHVTLFIYIAR | |
| | LYHHADPENRQGLEDLISSGVTIQIMTEQESGYCWRNFVNYSPSNEAHWPRYPHLWVRLYVLELYCII | |
| | LGLPPCLNILRRKQPQLTFFTIALQSCHYQRLPPHILWATGLKSGGSSGGSSGSETPGTSESATPESS | |
| | GGSSGGSDKKYSIGLAIGTNSVGWAVITDEYKVPSKKFKVLGNTDRHSIKKNLIGALLFDSGETAEAT | |
| | RLKRTARRRYTRRKNRICYLQEIFSNEMAKVDDSFFHRLEESFLVEEDKKHERHPIFGNIVDEVAYHE | |
| | KYPTIYHLRKKLVDSTDKADLRLIYLALAHMIKFRGHFLIEGDLNPDNSDVDKLFIQLVQTYNQLFEE | |
| | NPINASGVDAKAILSARLSKSRRLENLIAQLPGEKKNGLFGNLIALSLGLTPNFKSNFDLAEDAKLQL | |
| | SKDTYDDDLDNLLAQIGDQYADLFLAAKNLSDAILLSDILRVNTEITKAPLSASMIKRYDEHHQDLTL | |
| | LKALVRQQLPEKYKEIFFDQSKNGYAGYIDGGASQEEFYKFIKPILEKMDGTEELLVKLNREDLLRKQ | |
| | RTFDNGSIPHQIHLGELHAILRRQEDFYPFLKDNREKIEKILTFRIPYYVGPLARGNSRFAWMTRKSE | |
| | ETITPWNFEEVVDKGASAQSFIERMTNFDKNLPNEKVLPKHSLLYEYFTVYNELTKVKYVTEGMRKPA | |
| | FLSGEQKKAIVDLLFKTNRKVTVKQLKEDYFKKIECFDSVEISGVEDRFNASLGTYHDLLKIIKDKDF | |
| | LDNEENEDILEDIVLTLTLFEDREMIEERLKTYAHLFDDKVMKQLKRRRYTGWGRLSRKLINGIRDKQ | |
| | SGKTILDFLKSDGFANRNFMQLIHDDSLTFKEDIQKAQVSGQGDSLHEHIANLAGSPAIKKGILQTVK | |
| | VVDELVKVMGRHKPENIVIEMARENQTTQKGQKNSRERMKRIEEGIKELGSQILKEHPVENTQLQNEK | |
| | LYLYYLQNGRDMYVDQELDINRLSDYDVDHIVPQSFLKDDSIDNKVLTRSDKNRGKSDNVPSEEVVKK | |
| | MKNYWRQLLNAKLITQRKFDNLTKAERGGLSELDKAGFIKRQLVETRQITKHVAQILDSRMNTKYDEN | |
| | DKLIREVKVITLKSKLVSDFRKDFQFYKVREINNYHHAHDAYLNAVVGTALIKKYPKLESEFVYGDYK | |
| | VYDVRKMIAKSEQEIGKATAKYFFYSNIMNFFKTEITLANGEIRKRPLIETNGETGEIVWDKGRDFAT | |
| | VRKVLSMPQVNIVKKTEVQTGGFSKESIRPKRNSDKLIARKKDWDPKKYGGFVSPTVAYSVLVVAKVE | |
| | KGKSKKLKSVKELLGITIMERSSFEKNPIDFLEAKGYKEVKKDLIIKLPKYSLFELENGRKRMLASAR | |
| | FLQKGNELALPSKYVNFLYLASHYEKLKGSPEDNEQKQLFVEQHKHYLDEIIEQISEFSKRVILADAN | |
| | LDKVLSAYNKHRDKPIREQAENIIHLFTLTNLGAPRAFKYFDTTIDRKVYRSTKEVLDATLIHQSITG | |
| | LYETRIDLSQLGGDSGGSGGSGGSTNLSDIIEKETGKQLVIQESILMLPEEVEEVIGNKPESDILVHT | |
| | AYDESTDENVMLLTSDAPEYKPWALVIQDSNGENKIKMLSGGSGGSGGSTNLSDIIEKETGKQLVIQE | |
| | SILMLPEEVEEVIGNKPESDILVHTAYDESTDENVMLLTSDAPEYKPWALVIQDSNGENKIKMLSGGS | |
| | KRTADGSEFEPKKKRKV | |
| EE-SpCas9-NG | MKRTADGSEFESPKKKRKVSSETGPVAVDPTLRRRIEPHEFEVFFDPRELRKETCLLYEINWGGRHSI | 242 |
| base editor | WRHTSQNTNKHVEVNFIEKFTTERYFCPNTRCSITWFLSWSPCGECSRAITEFLSRYPHVTLFIYIAR | |
| | LYHHADPENRQGLEDLISSGVTIQIMTEQESGYCWRNFVNYSPSNEAHWPRYPHLWVRLYVLELYCII | |
| | LGLPPCLNILRRKQPQLTFFTIALQSCHYQRLPPHILWATGLKSGGSSGGSSGSETPGTSESATPESS | |
| | GGSSGGSDKKYSIGLAIGTNSVGWAVITDEYKVPSKKFKVLGNTDRHSIKKNLIGALLFDSGETAEAT | |
| | RLKRTARRRYTRRKNRICYLQEIFSNEMAKVDDSFFHRLEESFLVEEDKKHERHPIFGNIVDEVAYHE | |
| | KYPTIYHLRKKLVDSTDKADLRLIYLALAHMIKFRGHFLIEGDLNPDNSDVDKLFIQLVQTYNQLFEE | |
| | NPINASGVDAKAILSARLSKSRRLENLIAQLPGEKKNGLFGNLIALSLGLTPNFKSNFDLAEDAKLQL | |
| | SKDTYDDDLDNLLAQIGDQYADLFLAAKNLSDAILLSDILRVNTEITKAPLSASMIKRYDEHHQDLTL | |
| | LKALVRQQLPEKYKEIFFDQSKNGYAGYIDGGASQEEFYKFIKPILEKMDGTEELLVKLNREDLLRKQ | |
| | RTFDNGSIPHQIHLGELHAILRRQEDFYPFLKDNREKIEKILTFRIPYYVGPLARGNSRFAWMTRKSE | |
| | ETITPWNFEEVVDKGASAQSFIERMTNFDKNLPNEKVLPKHSLLYEYFTVYNELTKVKYVTEGMRKPA | |
| | FLSGEQKKAIVDLLFKTNRKVTVKQLKEDYFKKIECFDSVEISGVEDRFNASLGTYHDLLKIIKDKDF | |
| | LDNEENEDILEDIVLTLTLFEDREMIEERLKTYAHLFDDKVMKQLKRRRYTGWGRLSRKLINGIRDKQ | |
| | SGKTILDFLKSDGFANRNFMQLIHDDSLTFKEDIQKAQVSGQGDSLHEHIANLAGSPAIKKGILQTVK | |
| | VVDELVKVMGRHKPENIVIEMARENQTTQKGQKNSRERMKRIEEGIKELGSQILKEHPVENTQLQNEK | |
| | LYLYYLQNGRDMYVDQELDINRLSDYDVDHIVPQSFLKDDSIDNKVLTRSDKNRGKSDNVPSEEVVKK | |
| | MKNYWRQLLNAKLITQRKFDNLTKAERGGLSELDKAGFIKRQLVETRQITKHVAQILDSRMNTKYDEN | |
| | DKLIREVKVITLKSKLVSDFRKDFQFYKVREINNYHHAHDAYLNAVVGTALIKKYPKLESEFVYGDYK | |
| | VYDVRKMIAKSEQEIGKATAKYFFYSNIMNFFKTEITLANGEIRKRPLIETNGETGEIVWDKGRDFAT | |
| | VRKVLSMPQVNIVKKTEVQTGGFSKESIRPKRNSDKLIARKKDWDPKKYGGFVSPTVAYSVLVVAKVE | |
| | KGKSKKLKSVKELLGITIMERSSFEKNPIDFLEAKGYKEVKKDLIIKLPKYSLFELENGRKRMLASAR | |
| | FLQKGNELALPSKYVNFLYLASHYEKLKGSPEDNEQKQLFVEQHKHYLDEIIEQISEFSKRVILADAN | |
| | LDKVLSAYNKHRDKPIREQAENIIHLFTLTNLGAPRAFKYFDTTIDRKVYRSTKEVLDATLIHQSITG | |
| | LYETRIDLSQLGGDSGGSGGSGGSTNLSDIIEKETGKQLVIQESILMLPEEVEEVIGNKPESDILVHT | |
| | AYDESTDENVMLLTSDAPEYKPWALVIQDSNGENKIKMLSGGSGGSGGSTNLSDIIEKETGKQLVIQE | |
| | SILMLPEEVEEVIGNKPESDILVHTAYDESTDENVMLLTSDAPEYKPWALVIQDSNGENKIKMLSGGS | |
| | KRTADGSEFEPKKKRKV | |
| R33A + K34A- | MKRTADGSEFESPKKKRKVSSETGPVAVDPTLRRRIEPHEFEVFFDPRELAAETCLLYEINWGGRHSI | 243 |
| SpCas9-NG base | WRHTSQNTNKHVEVNFIEKFTTERYFCPNTRCSITWFLSWSPCGECSRAITEFLSRYPHVTLFIYIAR | |
| editor | LYHHADPRNRQGLRDLISSGVTIQIMTEQESGYCWRNFVNYSPSNEAHWPRYPHLWVRLYVLELYCII | |
| | LGLPPCLNILRRKQPQLTFFTIALQSCHYQRLPPHILWATGLKSGGSSGGSSGSETPGTSESATPESS | |
| | GGSSGGSDKKYSIGLAIGTNSVGWAVITDEYKVPSKKFKVLGNTDRHSIKKNLIGALLFDSGETAEAT | |
| | RLKRTARRRYTRRKNRICYLQEIFSNEMAKVDDSFFHRLEESFLVEEDKKHERHPIFGNIVDEVAYHE | |
| | KYPTIYHLRKKLVDSTDKADLRLIYLALAHMIKFRGHFLIEGDLNPDNSDVDKLFIQLVQTYNQLFEE | |
| | NPINASGVDAKAILSARLSKSRRLENLIAQLPGEKKNGLFGNLIALSLGLTPNFKSNFDLAEDAKLQL | |
| | SKDTYDDDLDNLLAQIGDQYADLFLAAKNLSDAILLSDILRVNTEITKAPLSASMIKRYDEHHQDLTL | |
| | LKALVRQQLPEKYKEIFFDQSKNGYAGYIDGGASQEEFYKFIKPILEKMDGTEELLVKLNREDLLRKQ | |
| | RTFDNGSIPHQIHLGELHAILRRQEDFYPFLKDNREKIEKILTFRIPYYVGPLARGNSRFAWMTRKSE | |
| | ETITPWNFEEVVDKGASAQSFIERMTNFDKNLPNEKVLPKHSLLYEYFTVYNELTKVKYVTEGMRKPA | |
| | FLSGEQKKAIVDLLFKTNRKVTVKQLKEDYFKKIECFDSVEISGVEDRFNASLGTYHDLLKIIKDKDF | |
| | LDNEENEDILEDIVLTLTLFEDREMIEERLKTYAHLFDDKVMKQLKRRRYTGWGRLSRKLINGIRDKQ | |
| | SGKTILDFLKSDGFANRNFMQLIHDDSLTFKEDIQKAQVSGQGDSLHEHIANLAGSPAIKKGILQTVK | |
| | VVDELVKVMGRHKPENIVIEMARENQTTQKGQKNSRERMKRIEEGIKELGSQILKEHPVENTQLQNEK | |
| | LYLYYLQNGRDMYVDQELDINRLSDYDVDHIVPQSFLKDDSIDNKVLTRSDKNRGKSDNVPSEEVVKK | |
| | MKNYWRQLLNAKLITQRKFDNLTKAERGGLSELDKAGFIKRQLVETRQITKHVAQILDSRMNTKYDEN | |
| | DKLIREVKVITLKSKLVSDFRKDFQFYKVREINNYHHAHDAYLNAVVGTALIKKYPKLESEFVYGDYK | |
| | VYDVRKMIAKSEQEIGKATAKYFFYSNIMNFFKTEITLANGEIRKRPLIETNGETGEIVWDKGRDFAT | |
| | VRKVLSMPQVNIVKKTEVQTGGFSKESIRPKRNSDKLIARKKDWDPKKYGGFVSPTVAYSVLVVAKVE | |
| | KGKSKKLKSVKELLGITIMERSSFEKNPIDFLEAKGYKEVKKDLIIKLPKYSLFELENGRKRMLASAR | |
| | FLQKGNELALPSKYVNFLYLASHYEKLKGSPEDNEQKQLFVEQHKHYLDEIIEQISEFSKRVILADAN | |
| | LDKVLSAYNKHRDKPIREQAENIIHLFTLTNLGAPRAFKYFDTTIDRKVYRSTKEVLDATLIHQSITG | |
| | LYETRIDLSQLGGDSGGSGGSGGSTNLSDIIEKETGKQLVIQESILMLPEEVEEVIGNKPESDILVHT | |
| | AYDESTDENVMLLTSDAPEYKPWALVIQDSNGENKIKMLSGGSGGSGGSTNLSDIIEKETGKQLVIQE | |
| | SILMLPEEVEEVIGNKPESDILVHTAYDESTDENVMLLTSDAPEYKPWALVIQDSNGENKIKMLSGGS | |
| | KRTADGSEFEPKKKRKV | |
| YE1-CP1028 | MKRTADGSEFESPKKKRKVSSETGPVAVDPTLRRRIEPHEFEVFFDPRELRKETCLLYEINWGGRHSI | 244 |
| base editor | WRHTSQNTNKHVEVNFIEKFTTERYFCPNTRCSITWFLSYSPCGECSRAITEFLSRYPHVTLFIYIAR | |
| (YE1-BE4- | LYHHADPENRQGLRDLISSGVTIQIMTEQESGYCWRNFVNYSPSNEAHWPRYPHLWVRLYVLELYCII | |
| CP1028, or | LGLPPCLNILRRKQPQLTFFTIALQSCHYQRLPPHILWATGLKSGGSSGGSSGSETPGTSESATPESS | |
| YE1-CP) | GGSSGGSEIGKATAKYFFYSNIMNFFKTEITLANGEIRKRPLIETNGETGEIVWDKGRDFATVRKVLS | |
| | MPQVNIVKKTEVQTGGFSKESILPKRNSDKLIARKKDWDPKKYGGFDSPTVAYSVLVVAKVEKGKSKK | |
| | LKSVKELLGITIMERSSFEKNPIDFLEAKGYKEVKKDLIIKLPKYSLFELENGRKRMLASAGELQKGN | |
| | ELALPSKYVNFLYLASHYEKLKGSPEDNEQKQLFVEQHKHYLDEIIEQISEFSKRVILADANLDKVLS | |
| | AYNKHRDKPIREQAENIIHLFTLTNLGAPAAFKYFDTTIDRKRYTSTKEVLDATLIHQSITGLYETRI | |
| | DLSQLGGDGGSGGSGGSGGSGGSGGSGGMDKKYSIGLAIGTNSVGWAVITDEYKVPSKKFKVLGNTDR | |
| | HSIKKNLIGALLFDSGETAEATRLKRTARRRYTRRKNRICYLQEIFSNEMAKVDDSFFHRLEESFLVE | |
| | EDKKHERHPIFGNIVDEVAYHEKYPTIYHLRKKLVDSTDKADLRLIYLALAHMIKFRGHFLIEGDLNP | |
| | DNSDVDKLFIQLVQTYNQLFEENPINASGVDAKAILSARLSKSRRLENLIAQLPGEKKNGLFGNLIAL | |
| | SLGLTPNFKSNFDLAEDAKLQLSKDTYDDDLDNLLAQIGDQYADLFLAAKNLSDAILLSDILRVNTEI | |
| | TKAPLSASMIKRYDEHHQDLTLLKALVRQQLPEKYKEIFFDQSKNGYAGYIDGGASQEEFYKFIKPIL | |
| | EKMDGTEELLVKLNREDLLRKQRTFDNGSIPHQIHLGELHAILRRQEDFYPFLKDNREKIEKILTFRI | |
| | PYYVGPLARGNSRFAWMTRKSEETITPWNFEEVVDKGASAQSFIERMTNFDKNLPNEKVLPKHSLLYE | |
| | YFTVYNELTKVKYVTEGMRKPAFLSGEQKKAIVDLLFKTNRKVTVKQLKEDYFKKIECFDSVEISGVE | |
| | DRFNASLGTYHDLLKIIKDKDFLDNEENEDILEDIVLTLTLFEDREMIEERLKTYAHLFDDKVMKQLK | |
| | RRRYTGWGRLSRKLINGIRDKQSGKTILDFLKSDGFANRNFMQLIHDDSLTFKEDIQKAQVSGQGDSL | |
| | HEHIANLAGSPAIKKGILQTVKVVDELVKVMGRHKPENIVIEMARENQTTQKGQKNSRERMKRIEEGI | |
| | KELGSQILKEHPVENTQLQNEKLYLYYLQNGRDMYVDQELDINRLSDYDVDHIVPQSFLKDDSIDNKV | |
| | LTRSDKNRGKSDNVPSEEVVKKMKNYWRQLLNAKLITQRKFDNLTKAERGGLSELDKAGFIKRQLVET | |
| | RQITKHVAQILDSRMNTKYDENDKLIREVKVITLKSKLVSDFRKDFQFYKVREINNYHHAHDAYLNAV | |
| | VGTALIKKYPKLESEFVYGDYKVYDVRKMIAKSEQSGGSGGSGGSTNLSDIIEKETGKQLVIQESILM | |
| | LPEEVEEVIGNKPESDILVHTAYDESTDENVMLLTSDAPEYKPWALVIQDSNGENKIKMLSGGSGGSG | |
| | GSTNLSDIIEKETGKQLVIQESILMLPEEVEEVIGNKPESDILVHTAYDESTDENVMLLTSDAPEYKP | |
| | WALVIQDSNGENKIKMLSGGSKRTADGSEFEPKKKRKV | |
| YE2-CP1028 | MKRTADGSEFESPKKKRKVSSETGPVAVDPTLRRRIEPHEFEVFFDPRELRKETCLLYEINWGGRHSI | 245 |
| base editor | WRHTSQNTNKHVEVNFIEKFTTERYFCPNTRCSITWFLSYSPCGECSRAITEFLSRYPHVTLFIYIAR | |
| (YE2-BE4- | LYHHADPRNRQGLEDLISSGVTIQIMTEQESGYCWRNFVNYSPSNEAHWPRYPHLWVRLYVLELYCII | |
| CP1028) | LGLPPCLNILRRKQPQLTFFTIALQSCHYQRLPPHILWATGLKSGGSSGGSSGSETPGTSESATPESS | |
| | GGSSGGSEIGKATAKYFFYSNIMNFFKTEITLANGEIRKRPLIETNGETGEIVWDKGRDFATVRKVLS | |
| | MPQVNIVKKTEVQTGGFSKESILPKRNSDKLIARKKDWDPKKYGGFDSPTVAYSVLVVAKVEKGKSKK | |
| | LKSVKELLGITIMERSSFEKNPIDFLEAKGYKEVKKDLIIKLPKYSLFELENGRKRMLASAGELQKGN | |
| | ELALPSKYVNFLYLASHYEKLKGSPEDNEQKQLFVEQHKHYLDEIIEQISEFSKRVILADANLDKVLS | |
| | AYNKHRDKPIREQAENIIHLFTLTNLGAPAAFKYFDTTIDRKRYTSTKEVLDATLIHQSITGLYETRI | |
| | DLSQLGGDGGSGGSGGSGGSGGSGGSGGMDKKYSIGLAIGTNSVGWAVITDEYKVPSKKFKVLGNTDR | |
| | HSIKKNLIGALLFDSGETAEATRLKRTARRRYTRRKNRICYLQEIFSNEMAKVDDSFFHRLEESFLVE | |
| | EDKKHERHPIFGNIVDEVAYHEKYPTIYHLRKKLVDSTDKADLRLIYLALAHMIKFRGHFLIEGDLNP | |
| | DNSDVDKLFIQLVQTYNQLFEENPINASGVDAKAILSARLSKSRRLENLIAQLPGEKKNGLFGNLIAL | |
| | SLGLTPNFKSNFDLAEDAKLQLSKDTYDDDLDNLLAQIGDQYADLFLAAKNLSDAILLSDILRVNTEI | |
| | TKAPLSASMIKRYDEHHQDLTLLKALVRQQLPEKYKEIFFDQSKNGYAGYIDGGASQEEFYKFIKPIL | |
| | EKMDGTEELLVKLNREDLLRKQRTFDNGSIPHQIHLGELHAILRRQEDFYPFLKDNREKIEKILTFRI | |
| | PYYVGPLARGNSRFAWMTRKSEETITPWNFEEVVDKGASAQSFIERMTNFDKNLPNEKVLPKHSLLYE | |
| | YFTVYNELTKVKYVTEGMRKPAFLSGEQKKAIVDLLFKTNRKVTVKQLKEDYFKKIECFDSVEISGVE | |
| | DRFNASLGTYHDLLKIIKDKDFLDNEENEDILEDIVLTLTLFEDREMIEERLKTYAHLFDDKVMKQLK | |
| | RRRYTGWGRLSRKLINGIRDKQSGKTILDFLKSDGFANRNFMQLIHDDSLTFKEDIQKAQVSGQGDSL | |
| | HEHIANLAGSPAIKKGILQTVKVVDELVKVMGRHKPENIVIEMARENQTTQKGQKNSRERMKRIEEGI | |
| | KELGSQILKEHPVENTQLQNEKLYLYYLQNGRDMYVDQELDINRLSDYDVDHIVPQSFLKDDSIDNKV | |
| | LTRSDKNRGKSDNVPSEEVVKKMKNYWRQLLNAKLITQRKFDNLTKAERGGLSELDKAGFIKRQLVET | |
| | RQITKHVAQILDSRMNTKYDENDKLIREVKVITLKSKLVSDFRKDFQFYKVREINNYHHAHDAYLNAV | |
| | VGTALIKKYPKLESEFVYGDYKVYDVRKMIAKSEQSGGSGGSGGSTNLSDIIEKETGKQLVIQESILM | |
| | LPEEVEEVIGNKPESDILVHTAYDESTDENVMLLTSDAPEYKPWALVIQDSNGENKIKMLSGGSGGSG | |
| | GSTNLSDIIEKETGKQLVIQESILMLPEEVEEVIGNKPESDILVHTAYDESTDENVMLLTSDAPEYKP | |
| | WALVIQDSNGENKIKMLSGGSKRTADGSEFEPKKKRKV | |
| YEE-CP1028 | MKRTADGSEFESPKKKRKVSSETGPVAVDPTLRRRIEPHEFEVFFDPRELRKETCLLYEINWGGRHSI | 246 |
| base editor | WRHTSQNTNKHVEVNFIEKFTTERYFCPNTRCSITWFLSYSPCGECSRAITEFLSRYPHVTLFIYIAR | |
| (YEE-BE4- | LYHHADPENRQGLEDLISSGVTIQIMTEQESGYCWRNFVNYSPSNEAHWPRYPHLWVRLYVLELYCII | |
| CP1028) | LGLPPCLNILRRKQPQLTFFTIALQSCHYQRLPPHILWATGLKSGGSSGGSSGSETPGTSESATPESS | |
| | GGSSGGSEIGKATAKYFFYSNIMNFFKTEITLANGEIRKRPLIETNGETGEIVWDKGRDFATVRKVLS | |
| | MPQVNIVKKTEVQTGGFSKESILPKRNSDKLIARKKDWDPKKYGGFDSPTVAYSVLVVAKVEKGKSKK | |
| | LKSVKELLGITIMERSSFEKNPIDFLEAKGYKEVKKDLIIKLPKYSLFELENGRKRMLASAGELQKGN | |
| | ELALPSKYVNFLYLASHYEKLKGSPEDNEQKQLFVEQHKHYLDEIIEQISEFSKRVILADANLDKVLS | |
| | AYNKHRDKPIREQAENIIHLFTLTNLGAPAAFKYFDTTIDRKRYTSTKEVLDATLIHQSITGLYETRI | |
| | DLSQLGGDGGSGGSGGSGGSGGSGGSGGMDKKYSIGLAIGTNSVGWAVITDEYKVPSKKFKVLGNTDR | |
| | HSIKKNLIGALLFDSGETAEATRLKRTARRRYTRRKNRICYLQEIFSNEMAKVDDSFFHRLEESFLVE | |
| | EDKKHERHPIFGNIVDEVAYHEKYPTIYHLRKKLVDSTDKADLRLIYLALAHMIKFRGHFLIEGDLNP | |
| | DNSDVDKLFIQLVQTYNQLFEENPINASGVDAKAILSARLSKSRRLENLIAQLPGEKKNGLFGNLIAL | |
| | SLGLTPNFKSNFDLAEDAKLQLSKDTYDDDLDNLLAQIGDQYADLFLAAKNLSDAILLSDILRVNTEI | |
| | TKAPLSASMIKRYDEHHQDLTLLKALVRQQLPEKYKEIFFDQSKNGYAGYIDGGASQEEFYKFIKPIL | |
| | EKMDGTEELLVKLNREDLLRKQRTFDNGSIPHQIHLGELHAILRRQEDFYPFLKDNREKIEKILTFRI | |
| | PYYVGPLARGNSRFAWMTRKSEETITPWNFEEVVDKGASAQSFIERMTNFDKNLPNEKVLPKHSLLYE | |
| | YFTVYNELTKVKYVTEGMRKPAFLSGEQKKAIVDLLFKTNRKVTVKQLKEDYFKKIECFDSVEISGVE | |
| | DRFNASLGTYHDLLKIIKDKDFLDNEENEDILEDIVLTLTLFEDREMIEERLKTYAHLFDDKVMKQLK | |
| | RRRYTGWGRLSRKLINGIRDKQSGKTILDFLKSDGFANRNFMQLIHDDSLTFKEDIQKAQVSGQGDSL | |
| | HEHIANLAGSPAIKKGILQTVKVVDELVKVMGRHKPENIVIEMARENQTTQKGQKNSRERMKRIEEGI | |
| | KELGSQILKEHPVENTQLQNEKLYLYYLQNGRDMYVDQELDINRLSDYDVDHIVPQSFLKDDSIDNKV | |
| | LTRSDKNRGKSDNVPSEEVVKKMKNYWRQLLNAKLITQRKFDNLTKAERGGLSELDKAGFIKRQLVET | |
| | RQITKHVAQILDSRMNTKYDENDKLIREVKVITLKSKLVSDFRKDFQFYKVREINNYHHAHDAYLNAV | |
| | VGTALIKKYPKLESEFVYGDYKVYDVRKMIAKSEQSGGSGGSGGSTNLSDIIEKETGKQLVIQESILM | |
| | LPEEVEEVIGNKPESDILVHTAYDESTDENVMLLTSDAPEYKPWALVIQDSNGENKIKMLSGGSGGSG | |
| | GSTNLSDIIEKETGKQLVIQESILMLPEEVEEVIGNKPESDILVHTAYDESTDENVMLLTSDAPEYKP | |
| | WALVIQDSNGENKIKMLSGGSKRTADGSEFEPKKKRKV | |
| EE-CP1028 base | MKRTADGSEFESPKKKRKVSSETGPVAVDPTLRRRIEPHEFEVFFDPRELRKETCLLYEINWGGRHSI | 247 |
| editor (EE- | WRHTSQNTNKHVEVNFIEKFTTERYFCPNTRCSITWFLSWSPCGECSRAITEFLSRYPHVTLFIYIAR | |
| BE4-CP1028) | LYHHADPENRQGLEDLISSGVTIQIMTEQESGYCWRNFVNYSPSNEAHWPRYPHLWVRLYVLELYCII | |
| | LGLPPCLNILRRKQPQLTFFTIALQSCHYQRLPPHILWATGLKSGGSSGGSSGSETPGTSESATPESS | |
| | GGSSGGSEIGKATAKYFFYSNIMNFFKTEITLANGEIRKRPLIETNGETGEIVWDKGRDFATVRKVLS | |
| | MPQVNIVKKTEVQTGGFSKESILPKRNSDKLIARKKDWDPKKYGGFDSPTVAYSVLVVAKVEKGKSKK | |
| | LKSVKELLGITIMERSSFEKNPIDFLEAKGYKEVKKDLIIKLPKYSLFELENGRKRMLASAGELQKGN | |
| | ELALPSKYVNFLYLASHYEKLKGSPEDNEQKQLFVEQHKHYLDEIIEQISEFSKRVILADANLDKVLS | |
| | AYNKHRDKPIREQAENIIHLFTLTNLGAPAAFKYFDTTIDRKRYTSTKEVLDATLIHQSITGLYETRI | |
| | DLSQLGGDGGSGGSGGSGGSGGSGGSGGMDKKYSIGLAIGTNSVGWAVITDEYKVPSKKFKVLGNTDR | |
| | HSIKKNLIGALLFDSGETAEATRLKRTARRRYTRRKNRICYLQEIFSNEMAKVDDSFFHRLEESFLVE | |
| | EDKKHERHPIFGNIVDEVAYHEKYPTIYHLRKKLVDSTDKADLRLIYLALAHMIKFRGHFLIEGDLNP | |
| | DNSDVDKLFIQLVQTYNQLFEENPINASGVDAKAILSARLSKSRRLENLIAQLPGEKKNGLFGNLIAL | |
| | SLGLTPNFKSNFDLAEDAKLQLSKDTYDDDLDNLLAQIGDQYADLFLAAKNLSDAILLSDILRVNTEI | |
| | TKAPLSASMIKRYDEHHQDLTLLKALVRQQLPEKYKEIFFDQSKNGYAGYIDGGASQEEFYKFIKPIL | |
| | EKMDGTEELLVKLNREDLLRKQRTFDNGSIPHQIHLGELHAILRRQEDFYPFLKDNREKIEKILTFRI | |
| | PYYVGPLARGNSRFAWMTRKSEETITPWNFEEVVDKGASAQSFIERMTNFDKNLPNEKVLPKHSLLYE | |
| | YFTVYNELTKVKYVTEGMRKPAFLSGEQKKAIVDLLFKTNRKVTVKQLKEDYFKKIECFDSVEISGVE | |
| | DRFNASLGTYHDLLKIIKDKDFLDNEENEDILEDIVLTLTLFEDREMIEERLKTYAHLFDDKVMKQLK | |
| | RRRYTGWGRLSRKLINGIRDKQSGKTILDFLKSDGFANRNFMQLIHDDSLTFKEDIQKAQVSGQGDSL | |
| | HEHIANLAGSPAIKKGILQTVKVVDELVKVMGRHKPENIVIEMARENQTTQKGQKNSRERMKRIEEGI | |
| | KELGSQILKEHPVENTQLQNEKLYLYYLQNGRDMYVDQELDINRLSDYDVDHIVPQSFLKDDSIDNKV | |
| | LTRSDKNRGKSDNVPSEEVVKKMKNYWRQLLNAKLITQRKFDNLTKAERGGLSELDKAGFIKRQLVET | |
| | RQITKHVAQILDSRMNTKYDENDKLIREVKVITLKSKLVSDFRKDFQFYKVREINNYHHAHDAYLNAV | |
| | VGTALIKKYPKLESEFVYGDYKVYDVRKMIAKSEQSGGSGGSGGSTNLSDIIEKETGKQLVIQESILM | |
| | LPEEVEEVIGNKPESDILVHTAYDESTDENVMLLTSDAPEYKPWALVIQDSNGENKIKMLSGGSGGSG | |
| | GSTNLSDIIEKETGKQLVIQESILMLPEEVEEVIGNKPESDILVHTAYDESTDENVMLLTSDAPEYKP | |
| | WALVIQDSNGENKIKMLSGGSKRTADGSEFEPKKKRKV | |
| R33A + K34A- | MKRTADGSEFESPKKKRKVSSETGPVAVDPTLRRRIEPHEFEVFFDPRELAAETCLLYEINWGGRHSI | 248 |
| CP1028 base | WRHTSQNTNKHVEVNFIEKFTTERYFCPNTRCSITWFLSWSPCGECSRAITEFLSRYPHVTLFIYIAR | |
| editor | LYHHADPRNRQGLRDLISSGVTIQIMTEQESGYCWRNFVNYSPSNEAHWPRYPHLWVRLYVLELYCII | |
| (R33A + K34A- | LGLPPCLNILRRKQPQLTFFTIALQSCHYQRLPPHILWATGLKSGGSSGGSSGSETPGTSESATPESS | |
| BE4-CP1028) | GGSSGGSEIGKATAKYFFYSNIMNFFKTEITLANGEIRKRPLIETNGETGEIVWDKGRDFATVRKVLS | |
| | MPQVNIVKKTEVQTGGFSKESILPKRNSDKLIARKKDWDPKKYGGFDSPTVAYSVLVVAKVEKGKSKK | |
| | LKSVKELLGITIMERSSFEKNPIDFLEAKGYKEVKKDLIIKLPKYSLFELENGRKRMLASAGELQKGN | |
| | ELALPSKYVNFLYLASHYEKLKGSPEDNEQKQLFVEQHKHYLDEIIEQISEFSKRVILADANLDKVLS | |
| | AYNKHRDKPIREQAENIIHLFTLTNLGAPAAFKYFDTTIDRKRYTSTKEVLDATLIHQSITGLYETRI | |
| | DLSQLGGDGGSGGSGGSGGSGGSGGSGGMDKKYSIGLAIGTNSVGWAVITDEYKVPSKKFKVLGNTDR | |
| | HSIKKNLIGALLFDSGETAEATRLKRTARRRYTRRKNRICYLQEIFSNEMAKVDDSFFHRLEESFLVE | |
| | EDKKHERHPIFGNIVDEVAYHEKYPTIYHLRKKLVDSTDKADLRLIYLALAHMIKFRGHFLIEGDLNP | |
| | DNSDVDKLFIQLVQTYNQLFEENPINASGVDAKAILSARLSKSRRLENLIAQLPGEKKNGLFGNLIAL | |
| | SLGLTPNFKSNFDLAEDAKLQLSKDTYDDDLDNLLAQIGDQYADLFLAAKNLSDAILLSDILRVNTEI | |
| | TKAPLSASMIKRYDEHHQDLTLLKALVRQQLPEKYKEIFFDQSKNGYAGYIDGGASQEEFYKFIKPIL | |
| | EKMDGTEELLVKLNREDLLRKQRTFDNGSIPHQIHLGELHAILRRQEDFYPFLKDNREKIEKILTFRI | |
| | PYYVGPLARGNSRFAWMTRKSEETITPWNFEEVVDKGASAQSFIERMTNFDKNLPNEKVLPKHSLLYE | |
| | YFTVYNELTKVKYVTEGMRKPAFLSGEQKKAIVDLLFKTNRKVTVKQLKEDYFKKIECFDSVEISGVE | |
| | DRFNASLGTYHDLLKIIKDKDFLDNEENEDILEDIVLTLTLFEDREMIEERLKTYAHLFDDKVMKQLK | |
| | RRRYTGWGRLSRKLINGIRDKQSGKTILDFLKSDGFANRNFMQLIHDDSLTFKEDIQKAQVSGQGDSL | |
| | HEHIANLAGSPAIKKGILQTVKVVDELVKVMGRHKPENIVIEMARENQTTQKGQKNSRERMKRIEEGI | |
| | KELGSQILKEHPVENTQLQNEKLYLYYLQNGRDMYVDQELDINRLSDYDVDHIVPQSFLKDDSIDNKV | |
| | LTRSDKNRGKSDNVPSEEVVKKMKNYWRQLLNAKLITQRKFDNLTKAERGGLSELDKAGFIKRQLVET | |
| | RQITKHVAQILDSRMNTKYDENDKLIREVKVITLKSKLVSDFRKDFQFYKVREINNYHHAHDAYLNAV | |
| | VGTALIKKYPKLESEFVYGDYKVYDVRKMIAKSEQSGGSGGSGGSTNLSDIIEKETGKQLVIQESILM | |
| | LPEEVEEVIGNKPESDILVHTAYDESTDENVMLLTSDAPEYKPWALVIQDSNGENKIKMLSGGSGGSG | |
| | GSTNLSDIIEKETGKQLVIQESILMLPEEVEEVIGNKPESDILVHTAYDESTDENVMLLTSDAPEYKP | |
| | WALVIQDSNGENKIKMLSGGSKRTADGSEFEPKKKRKV | |

guide RNAs and target DNA sequences

| | | SEQ ID |
|---|---|---|
| DESCRIPTION | SEQUENCE | NO: |
| Portion of | GGTTTCAGACAAAATCAAAAAGAAGGAAGGTGCTCACATTCCTTAAATTAA | 249 |
| SMN2 gene with | | |
| C840 residue | | |
| in bold within | | |
| position 6 of | | |
| exon 7 (active | | |
| splice site) | | |
| | AUUUUGUCUAAAACCCUGUA | 250 |
| | GGTTTTAGACAAAATCAAAAAGAAGGAAGGTGCTCACATTCCTTAAATTAA | 251 |
| Portion of | ATTTTCCTTACAGGGTTTTA | 252 |
| SMN2 gene with | | |
| C840T mutation | | |
| in bold within | | |
| position 6 of | | |
| exon 7 | | |
| SMN2 | TTTCCTTACAGGGTTTTAGA | 253 |
| SMN2 | TTCCTTACAGGGTTTTAGAC | 254 |
| SMN2 | TCCTTACAGGGTTTTAGACA | 255 |
| SMN2 | CCTTACAGGGTTTTAGACAA | 256 |
| SMN2 | CTTACAGGGTTTTAGACAAA | 257 |
| SMN2 | TTACAGGGTTTTAGACAAAA | 258 |
| SMN2 | TACAGGGTTTTAGACAAAAT | 259 |
| SMN2 | ACAGGGTTTTAGACAAAATC | 260 |
| SMN2 | GTTTTAGACAAAATC | 261 |
| SMN2 | GGTTTTAGACAAAATCA | 262 |
| SMN2 | GGGTTTTAGACAAAATCAA | 263 |
| SMN2 | AGGGTTTTAGACAAAATCAAA | 264 |
| SMN2 | CAGGGTTTTAGACAAAATCAAAA | 265 |
| SMN2 | ACAGGGTTTTAGACAAAATCAAAA | 266 |
| SMN2 | CATAGAGCAGCACTAAATG | 267 |
| SMN2 | ATAGAGCAGCACTAAATGA | 268 |
| SMN2 | TAGAGCAGCACTAAATGAC | 269 |
| SMN2 | AGAGCAGCACTAAATGACA | 270 |
| SMN2 | GAGCAGCACTAAATGACAC | 271 |
| SMN2 | AGCAGCACTAAATGACACC | 272 |
| SMN2 | GCAGCACTAAATGACACCA | 273 |
| SMN2 | CAGCACTAAATGACACCAT | 274 |
| SMN2 | AGCACTAAATGACACCATA | 275 |
| SMN2 | GCACTAAATGACACCATAA | 276 |
| SMN2 | TAAATGACACCATAA | 277 |
| SMN2 | CTAAATGACACCATAAA | 278 |
| SMN2 | ACTAAATGACACCATAAAG | 279 |
| SMN2 | CACTAAATGACACCATAAAGA | 280 |
| SMN2 | GCACTAAATGACACCATAAAGAA | 281 |
| SMN2 | AGCACTAAATGACACCATAAAGAAA | 282 |
| SMN2 | AATTTCATGGTACATGAGTG | 283 |
| SMN2 | TTTCATGGTACATGAGTGGC | 284 |
| SMN2 | TTCATGGTACATGAGTGGCT | 285 |
| SMN2 | TCATGGTACATGAGTGGCTA | 286 |
| SMN2 | CATGGTACATGAGTGGCTAT | 287 |
| SMN2 | ATGGTACATGAGTGGCTATC | 288 |
| SMN2 | TGGTACATGAGTGGCTATCA | 289 |
| SMN2 | GGTACATGAGTGGCTATCAT | 290 |
| SMN2 | GTACATGAGTGGCTATCATA | 291 |
| SMN2 | TGAGTGGCTATCATA | 292 |
| SMN2 | ATGAGTGGCTATCATAC | 293 |
| SMN2 | CATGAGTGGCTATCATACT | 294 |
| SMN2 | ACATGAGTGGCTATCATACTG | 295 |
| SMN2 | TACATGAGTGGCTATCATACTGG | 296 |
| SMN2 | TTTTCCTTACAGGGTTTTAG | 398 |
| SMN2 | ATTTCATGGTACATGAGTGG | 399 |
| guide | (1) | 297 |
| | NNNNNNNNgtttttgtactctcaagatttaGAAAtaaatcttgcagaagctacaaagataaggctt | |
| | catgccgaaatcaacaccctgtcattttatggcagggtgttttcgttatttaaTTTTTT | |
| guide | (2) | 298 |
| | NNNNNNNNNNNNNNNNNNgtttttgtactctcaGAAAtgcagaagctacaaagataaggcttcatgcc | |
| | gaaatca acaccctgtcattttatggcagggtgttttcgttatttaaTTTTTT | |
| guide | (3) | 299 |
| | NNNNNNNNNNNNNNNNNNNNgtttttgtactctcaGAAAtgcagaagctacaaagataaggcttcatg | |
| | ccgaaatca acaccctgtcattttatggcagggtgtTTTTT | |
| guide | (4) | 300 |
| | NNNNNNNNNNNNNNNNNNNNgttttagagctaGAAAtagcaagttaaaataaggctagtccgttatca | |
| | acttgaaaa agtggcaccgagtcggtgcTTTTTT | |
| guide | (5) | 301 |
| | NNNNNNNNNNNNNNNNNNNNgttttagagctaGAAATAGcaagttaaaataaggctagtccgttatca | |
| | acttgaa aaagtgTTTTTTT | |
| guide | (6) | 302 |
| | NNNNNNNNNNNNNNNNNNNNgttttagagctagAAATAGcaagttaaaataaggctagtccgttatca | |
| guide | GUUUUAGAGCUAGAAAUAGCAAGUUAAAAUAAAGGCUAGUCCGUUAUCAACUUGAAAAAGUGGCACCG | 303 |
| | AGUCGGUGCUUUUU | |
| guide | GUUUUAGAGCUAGAAAUAGCAAGUUAAAAUAAGGCUAGUCCGUUAUCAACUUGAAAAAGUGGCACCGA | 304 |
| | GUCGGUGCUUUUUUU | |
| guide | GGUCCACCCACCUGGGCUCCGUUUUAGAGCUAGAAAUAGCAAGUUAAAAUAAGGCUAGUCCGUUAUCA | 305 |
| | ACUUGAAAAAGUGGCACCGAGUCGGUGCUUUUUUU | |
| guide | AUUUUGUCUAAAACCCUGUAGUUUUAGAGCUAGAAAUAGCAAGUUAAAAUAAAGGCUAGUCCGUUAUC | 405 |
| | AACUUGAAAAAGUGGCACCGAGUCGGUGCUUUUU | |
| guide | AUUUUGUCUAAAACCCUGUAGUUUUAGAGCUAGAAAUAGCAAGUUAAAAUAAGGCUAGUCCGUUAUCA | 406 |
| | ACUUGAAAAAGUGGCACCGAGUCGGUGCUUUUUUU | |
| Exemplary | UUUUUGAUUUUGUCUAAAACCCUGUA | 306 |
| guide | | |
| sequences to | | |
| target a C840T | | |
| point mutation | | |
| in SMN2 | | |
| | UUUUGAUUUUGUCUAAAACCCUGUA | 307 |
| | UUUGAUUUUGUCUAAAACCCUGUA | 308 |
| | UUGAUUUUGUCUAAAACCCUGUA | 309 |
| | UGAUUUUGUCUAAAACCCUGUA | 310 |
| | GAUUUUGUCUAAAACCCUGUA | 311 |
| | AUUUUGUCUAAAACCCUGUA | 312 |
| | UUUUGUCUAAAACCCUGUA | 313 |
| | UUUGUCUAAAACCCUGUA | 314 |
| | UUGUCUAAAACCCUGUA | 315 |
| | UGUCUAAAACCCUGUA | 316 |
| | UUUGUCUAAAACCCUGUAAG | 317 |
| | UUUUGUCUAAAACCCUGUAA | 318 |
| | UGAUUUUGUCUAAAACCC | 319 |
| | GAUUUUGUCUAAAACCCU | 320 |
| | AUUUUGUCUAAAACCCUG | 321 |
| | GUCUAAAACCCUGUAAGG | 322 |
| | UCUAAAACCCUGUAAGGA | 323 |
| Exemplary | UUUGCAGGAAAUGCUGGCAU | 324 |
| guide | | |
| sequences to | | |
| target a stop | | |
| codon in exon | | |
| 8 of SMN2. | | |
| the A that is | | |
| complementary | | |
| to a stop | | |
| codon | | |
| comprising T | | |
| of exon 8 in | | |
| SMN2 is shown | | |
| in bold | | |
| | UUCUCAUUUGCAGGAAAUGC | 325 |
| | CAUUUAGUGCUGCUCUAUGC | 326 |
| | CAGGAAAUGCUGGCAUAGAG | 327 |
| | UUGCAGGAAAUGCUGGCAUA | 328 |
| | AUUUGCAGGAAAUGCUGGCA | 329 |
| Exemplary | UACAUGAGUGGCUAUCAUAC | 330 |
| guide | | |
| sequences to | | |
| target the | | |
| S270 amino | | |
| acid in exon 6 | | |
| of SMN2. | | |
| guide | UGAGCCGCUG | 400 |
| guide | UGAGCCGCUGG | 401 |
| guide | ATTTTGTCTAAAACCCTGTA | 331 |
| guide | AUUUUGUCUAAAACCcugua | 332 |
| guide | TTTGTCTAAAACCCTGTAAG | 333 |
| guide | TTTTGTCTAAAACCCTGTAA | 334 |
| guide | TGATTTTGTCTAAAACCC | 335 |
| guide | GATTTTGTCTAAAACCCT | 336 |
| guide | ATTTTGTCTAAAACCCTG | 337 |
| guide | GTCTAAAACCCTGTAAGG | 338 |
| guide | TCTAAAACCCTGTAAGGA | 339 |
| guide | UUUGUCUAAAACCCUGUAAG | 340 |
| guide | UUUUGUCUAAAACCCUGUAA | 341 |
| guide | UGAUUUUGUCUAAAACCC | 342 |
| guide | GAUUUUGUCUAAAACCCU | 343 |
| guide | AUUUUGUCUAAAACCCUG | 344 |
| guide | GUCUAAAACCCUGUAAGG | 345 |
| guide | UCUAAAACCCUGUAAGGA | 346 |
| guide | TTTGCAGGAAATGCTGGCAT | 347 |
| guide | TTCTCATTTGCAGGAAATGC | 348 |
| guide | CATTTAGTGCTGCTCTATGC | 349 |
| guide | CAGGAAATGCTGGCATAGAG | 350 |
| guide | TTGCAGGAAATGCTGGCATA | 351 |
| guide | ATTTGCAGGAAATGCTGGCA | 352 |
| guide | TGGCATAGAGCAGCACTAAA | 353 |
| guide | UUUGCAGGAAAUGCUGGCAU | 354 |
| guide | UUCUCAUUUGCAGGAAAUGC | 355 |
| guide | CAUUUAGUGCUGCUCUAUGC | 356 |
| guide | CAGGAAAUGCUGGCAUAGAG | 357 |
| guide | UUGCAGGAAAUGCUGGCAUA | 358 |
| guide | AUUUGCAGGAAAUGCUGGCA | 359 |
| guide | UGGCAUAGAGCAGCACUAAA | 360 |
| guide | TGGCATAGAGCAGCACTAAA | 361 |
| guide | UGGCAUAGAGCAGCACUAAA | 362 |
| genomic | Gtgaaacaaaatgctttttaacatccatataaagctatctatatatagctatctatatctatatagct | 363 |
| sequence of | attttttttaacttcctttattttccttacagGGTTTTAGACAAAATCAAAAAGAAGGAAGGTGCTCA | |
| the SMN2 exon | CATTCCTTAAATTAAggagtaagtctgccagcattatgaaagtgaatcttacttttgtaaaactttat | |
| 7 is presented | ggtttgtggaaaacaaatgtttttgaacatttaaaaagttcagatgt | |
| below (the | | |
| C→T mutation | | |
| is bolded and | | |
| underlined; | | |
| capitalization | | |
| represents the | | |
| exon) | | |
| Exon 7- | ATTTTGTCTAAAACCctgta | 364 |
| modifying | | |
| sgRNA (or | | |
| corresponding | | |
| DNA) | | |
| | AUUUUGUCUAAAACCcUgUa | 365 |
| | TTTGTCTAAAACCctgtaag | 366 |
| | UUUGUCUAAAACCcUgUaag | 367 |
| | TTTTGTCTAAAACCctgtaa | 368 |
| | UUUUGUCUAAAACCcUgUaa | 369 |
| | TGATTTTGTCTAAAACCC | 370 |
| | UGAUUUUGUCUAAAACCC | 371 |
| | GATTTTGTCTAAAACCCT | 372 |
| | GAUUUUGUCUAAAACCCU | 373 |
| | ATTTTGTCTAAAACCCTG | 374 |
| | AUUUUGUCUAAAACCCUG | 375 |
| | GTCTAAAACCCTGTAAGG | 376 |
| | GUCUAAAACCCUGUAAGG | 377 |
| | TCTAAAACCCTGTAAGGA | 378 |
| | UCUAAAACCCUGUAAGGA | 379 |
| Exon 8 | CtctggttctaatttctcatttgcagGAAATGCTGGCATAGAGCAGCACTAAATGACACCACTAAAGA | 380 |
| sequence (stop | AACGATCA | |
| codos are | | |
| bolded) | | |
| Exon 8- | TTTGCAGGAAATGCTGGCAT | 381 |
| modifying | | |
| sgRNAs | | |
| Exon 8- | UUUGCAGGAAAUGCUGGCAU | 382 |
| modifying | | |
| sgRNA | | |
| | TTCTCATTTGCAGGAAATGC | 383 |
| | UUCUCAUUUGCAGGAAAUGC | 384 |
| | CATTTAGTGCTGCTCTATGC | 385 |
| | CAUUUAGUGCUGCUCUAUGC | 386 |
| | CAGGAAATGCTGGCATAGAG | 387 |
| | CAGGAAAUGCUGGCAUAGAG | 388 |
| | TTGCAGGAAATGCTGGCATA | 389 |
| | UUGCAGGAAAUGCUGGCAUA | 390 |
| | ATTTGCAGGAAATGCTGGCA | 391 |
| | AUUUGCAGGAAAUGCUGGCA | 392 |
| | TGGCATAGAGCAGCACTAAA | 393 |
| | UGGCAUAGAGCAGCACUAAA | 394 |
| Exon 6 genomic | CTTTGGGAAGTATGTTAATTTCATGGTACATGAGTGGCTATCATACTGGCTATTATATGgtaagtaat | 395 |
| sequence (3270 | cactcagcatcttttcctgacaatttttttgtagttatgtgactttgttttgtaaattt | |
| is bolded) | | |
| sgRNA for ABE- | TACATGAGTGGCTATCATAC | 396 |
| mediated | | |
| codon- | | |
| switching in | | |
| exon 6 to | | |
| increase | | |
| stability of | | |
| SMN2 protein | | |
| | UACAUGAGUGGCUAUCAUAC | 397 |
| sgRNA | GTCTAAAACCCTGTAAGGAA | 408 |
| sgRNA | TGTCTAAAACCCTGTAAGGA | 409 |
| sgRNA | TTGTCTAAAACCCTGTAAGG | 410 |
| sgRNA | ATTTTGTCTAAAACCCTGTAAGG | 411 |
| sgRNA | GATTTTGTCTAAAACCCTGTAAG | 412 |
| sgRNA | TGATTTTGTCTAAAACCCTGTAA | 413 |
| sgRNA | GAAACCctgtaaggaaaataa | 414 |
| sgRNA | GTTTGTCTAAAACCctgtaag | 415 |
| sgRNA | GTTTTGTCTAAAACCctgtaa | 416 |
| sgRNA | GTGAGCACCTTCCTTCTTTT | 417 |
| sgRNA | GATGTGAGCACCTTCCTTCTT | 418 |
| sgRNA | GactccTTAATTTAAGGAATG | 419 |
| sgRNA | GcagacttactccTTAATTTA | 420 |
| sgRNA | GcagacttactccTTAATTTA | 421 |
| sgRNA | Gtaatgctggcagacttactc | 422 |
| sgRNA | Gttcactttcataatgctggc | 423 |
| sgRNA | Gaagattcactttcataatgc | 424 |
| sgRNA | Gacaaaagtaagattcacttt | 425 |
| sgRNA | Gacttcctttattttccttac | 426 |
| sgRNA | Gaacttcctttattttcctta | 427 |
| sgRNA | GtttccttacagGGTTTTAGA | 428 |
| sgRNA | GTTTAGACAAAATCAAAAAGA | 429 |
| sgRNA | GTTAGACAAAATCAAAAAGAA | 430 |
| sgRNA | GTAGACAAAATCAAAAAGAAG | 431 |
| sgRNA | GACAAAATCAAAAAGAAGGA | 432 |
| sgRNA | GAAGGAAGGTGCTCACATTCC | 433 |
| sgRNA | GTGCTCACATTCCTTAAATTA | 434 |
| sgRNA | GTGCTCACATTCCTTAAATT | 435 |
| sgRNA | GTGCTCACATTCCTTAAATTA | 436 |
| sgRNA | GCACATTCCTTAAATTAAgga | 437 |
| sgRNA | gagtaagtctgccagcatta | 438 |
| sgRNA | agtaagtctgccagcattat | 439 |
| sgRNA | gtctgccagcattatgaaag | 440 |
| sgRNA | Gagtctgccagcattatgaaa | 441 |
| sgRNA | Gcttacttttgtaaaacttta | 442 |
| sgRNA | Gttgtaaaactttatggtttg | 443 |
| sgRNA | aagcctctggttctaatttctcatttgcagGAAATGCTGGCATAGAGCAGCACTAAATGACACCACTA | 444 |
| | AAGAAACGATCAG | |
| sgRNA | GTTTCctgcaaatgagaaatt | 445 |
| sgRNA | GCCAGCATTTCctgcaaatg | 446 |
| sgRNA | GATGCCAGCATTTCctgcaaa | 447 |
| sgRNA | GCTCTATGCCAGCATTTCct | 448 |
| sgRNA | GAATGCTGGCATAGAGCAGCA | 449 |
| sgRNA | GTGGCATAGAGCAGCACTAAA | 450 |

Base editors used to generate training data for the BE-Hive Algorithm of Example 1

| | | |
|---|---|---|
| | | SEQ ID |
| DESCRIPTION | SEQUENCE | NO: |

The following CBEs were used to generate training data for the BE-Hive algorithm of Example 1.
Each of the CBEs have the same architecture of [NLS]-[deaminase]-[Cas 9]-[UGI]-[UGI]-[NLS]
(which is the BE4max architecture) and with interchangeable deaminases.
In addition, Cas-protein components of these editors can include SpCas9, SpCas9 circular
permutant 1028, or Cas9-NG. Amino acid sequences are provided for the BE4 (BE4max) construct as
an example, and separately amino acid sequences for deaminases and Cas9 proteins are provided
below.
Key:

| NLS (N-terminal) | Single underline | |
|---|---|---|
| APOBEC1 (BE4) | Double underline | |
| Linker | Italic | |
| SpCas9 | Plain | |
| Linker + 2xUGI | Bold underline | |
| NLS (C-terminal) | Single underline + italic | |
| BE4max (or BE4) | MKRTADGSEFESPKKKRKVSSETGPVAVDPTLRRRIEPHEFEVFFDPRELRKETCLLYE | 3200 |
| Cas9 = SpCas9 | INWGGRHSIWRHTSQNTNKHVEVNFIEKFTTERYFCPNTRCSITWFLSWSPCGECSRAI | |
| | TEFLSRYPHVTLFIYIARLYHHADPRNRQGLRDLISSGVTIQIMTEQESGYCWRNFVNY | |
| | SPSNEAHWPRYPHLWVRLYVLELYCIILGLPPCLNILRRKQPQLTFFTIALQSCHYQRL | |
| | PPHILWATGLKSGGSSGGSSGSETPGTSESATPESSGGSSGGDKKYSIGLAIGTNSVGW | |
| | AVITDEYKVPSKKFKVLGNTDRHSIKKNLIGALLFDSGETAEATRLKRTARRRYTRRKN | |
| | RICYLQEIFSNEMAKVDDSFFHRLEESFLVEEDKKHERHPIFGNIVDEVAYHEKYPTIY | |
| | HLRKKLVDSTDKADLRLIYLALAHMIKFRGHFLIEGDLNPDNSDVDKLFIQLVQTYNQL | |
| | FEENPINASGVDAKAILSARLSKSRRLENLIAQLPGEKKNGLFGNLIALSLGLTPNFKS | |
| | NFDLAEDAKLQLSKDTYDDDLDNLLAQIGDQYADLFLAAKNLSDAILLSDILRVNTEIT | |
| | KAPLSASMIKRYDEHHQDLTLLKALVRQQLPEKYKEIFFDQSKNGYAGYIDGGASQEEF | |
| | YKFIKPILEKMDGTEELLVKLNREDLLRKQRTFDNGSIPHQIHLGELHAILRRQEDFYP | |
| | FLKDNREKIEKILTFRIPYYVGPLARGNSRFAWMTRKSEETITPWNFEEVVDKGASAQS | |
| | FIERMTNFDKNLPNEKVLPKHSLLYEYFTVYNELTKVKYVTEGMRKPAFLSGEQKKAIV | |
| | DLLFKTNRKVTVKQLKEDYFKKIECFDSVEISGVEDRFNASLGTYHDLLKIIKDKDFLD | |
| | NEENEDILEDIVLTLTLFEDREMIEERLKTYAHLFDDKVMKQLKRRRYTGWGRLSRKLI | |
| | NGIRDKQSGKTILDFLKSDGFANRNFMQLIHDDSLTFKEDIQKAQVSGQGDSLHEHIAN | |
| | LAGSPAIKKGILQTVKVVDELVKVMGRHKPENIVIEMARENQTTQKGQKNSRERMKRIE | |
| | EGIKELGSQILKEHPVENTQLQNEKLYLYYLQNGRDMYVDQELDINRLSDYDVDHIVPQ | |
| | SFLKDDSIDNKVLTRSDKNRGKSDNVPSEEVVKKMKNYWRQLLNAKLITQRKFDNLTKA | |
| | ERGGLSELDKAGFIKRQLVETRQITKHVAQILDSRMNTKYDENDKLIREVKVITLKSKL | |
| | VSDFRKDFQFYKVREINNYHHAHDAYLNAVVGTALIKKYPKLESEFVYGDYKVYDVRKM | |
| | IAKSEQEIGKATAKYFFYSNIMNFFKTEITLANGEIRKRPLIETNGETGEIVWDKGRDF | |
| | ATVRKVLSMPQVNIVKKTEVQTGGFSKESILPKRNSDKLIARKKDWDPKKYGGFDSPTV | |
| | AYSVLVVAKVEKGKSKKLKSVKELLGITIMERSSFEKNPIDFLEAKGYKEVKKDLIIKL | |
| | PKYSLFELENGRKRMLASAGELQKGNELALPSKYVNFLYLASHYEKLKGSPEDNEQKQL | |
| | FVEQHKHYLDEIIEQISEFSKRVILADANLDKVLSAYNKHRDKPIREQAENIIHLFTLT | |
| | NLGAPAAFKYFDTTIDRKRYTSTKEVLDATLIHQSITGLYETRIDLSQLGGD [symbol] | |
| | [symbol] [symbol] [symbol] | |
| | [symbol] [symbol] [symbol] | |
| | [symbol] [symbol] [symbol] [symbol] | |
| | KRTADGSEFEPKKKRKV | |
| EA-BE4 | MKRTADGSEFESPKKKRKVSSETGPVAVDPTLRRRIEPHEFEVFFDPRELRKETCLLYE | 3201 |
| Cas9 = SpCas9 | INWGGREAIWRHTSQNTNKHVEVNFIEKFTTERYFCPNTRCSITWFLSWSPCGECSRAI | |
| | TEFLSRYPHVTLFIYIARLYHHADPRNRQGLRDLISSGVTIQIMTEQESGYCWRNFVNY | |
| | SPSNEAHWPRYPHLWVRLYVLELYCIILGLPPCLNILRRKQPQLTFFTIALQSCHYQRL | |
| | PPHILWATGLKSGGSSGGSSGSETPGTSESATPESSGGSSGGDKKYSIGLAIGTNSVGW | |
| | AVITDEYKVPSKKFKVLGNTDRHSIKKNLIGALLFDSGETAEATRLKRTARRRYTRRKN | |
| | RICYLQEIFSNEMAKVDDSFFHRLEESFLVEEDKKHERHPIFGNIVDEVAYHEKYPTIY | |
| | HLRKKLVDSTDKADLRLIYLALAHMIKFRGHFLIEGDLNPDNSDVDKLFIQLVQTYNQL | |
| | FEENPINASGVDAKAILSARLSKSRRLENLIAQLPGEKKNGLFGNLIALSLGLTPNFKS | |
| | NFDLAEDAKLQLSKDTYDDDLDNLLAQIGDQYADLFLAAKNLSDAILLSDILRVNTEIT | |
| | KAPLSASMIKRYDEHHQDLTLLKALVRQQLPEKYKEIFFDQSKNGYAGYIDGGASQEEF | |
| | YKFIKPILEKMDGTEELLVKLNREDLLRKQRTFDNGSIPHQIHLGELHAILRRQEDFYP | |
| | FLKDNREKIEKILTFRIPYYVGPLARGNSRFAWMTRKSEETITPWNFEEVVDKGASAQS | |
| | FIERMTNFDKNLPNEKVLPKHSLLYEYFTVYNELTKVKYVTEGMRKPAFLSGEQKKAIV | |
| | DLLFKTNRKVTVKQLKEDYFKKIECFDSVEISGVEDRFNASLGTYHDLLKIIKDKDFLD | |
| | NEENEDILEDIVLTLTLFEDREMIEERLKTYAHLFDDKVMKQLKRRRYTGWGRLSRKLI | |
| | NGIRDKQSGKTILDFLKSDGFANRNFMQLIHDDSLTFKEDIQKAQVSGQGDSLHEHIAN | |
| | LAGSPAIKKGILQTVKVVDELVKVMGRHKPENIVIEMARENQTTQKGQKNSRERMKRIE | |
| | EGIKELGSQILKEHPVENTQLQNEKLYLYYLQNGRDMYVDQELDINRLSDYDVDHIVPQ | |
| | SFLKDDSIDNKVLTRSDKNRGKSDNVPSEEVVKKMKNYWRQLLNAKLITQRKFDNLTKA | |
| | ERGGLSELDKAGFIKRQLVETRQITKHVAQILDSRMNTKYDENDKLIREVKVITLKSKL | |
| | VSDFRKDFQFYKVREINNYHHAHDAYLNAVVGTALIKKYPKLESEFVYGDYKVYDVRKM | |
| | IAKSEQEIGKATAKYFFYSNIMNFFKTEITLANGEIRKRPLIETNGETGEIVWDKGRDF | |
| | ATVRKVLSMPQVNIVKKTEVQTGGFSKESILPKRNSDKLIARKKDWDPKKYGGFDSPTV | |
| | AYSVLVVAKVEKGKSKKLKSVKELLGITIMERSSFEKNPIDFLEAKGYKEVKKDLIIKL | |
| | PKYSLFELENGRKRMLASAGELQKGNELALPSKYVNFLYLASHYEKLKGSPEDNEQKQL | |
| | FVEQHKHYLDEIIEQISEFSKRVILADANLDKVLSAYNKHRDKPIREQAENIIHLFTLT | |
| | NLGAPAAFKYFDTTIDRKRYTSTKEVLDATLIHQSITGLYETRIDLSQLGGD [symbol] | |
| | [symbol] [symbol] [symbol] [symbol] | |
| | [symbol] [symbol] [symbol] [symbol] | |
| | [symbol] [symbol] [symbol] [symbol] | |
| | [symbol] KRTADGSEFEPKKKRKV | |
| AID-BE4 | MKRTADGSEFESPKKKRKVDSLLMNRRKFLYQFKNVRWAKGRRETYLCYVVKRRDSATS | 3202 |
| Cas9 = SpCas9 | FSLDFGYLRNKNGCHVELLFLRYISDWDLDPGRCYRVTWFTSWSPCYDCARHVADFLRG | |
| | NPNLSLRIFTARLYFCEDRKAEPEGLRRLHRAGVQIAIMTFKDYFYCWNTFVENHERTF | |
| | KAWEGLHENSVRLSRQLRRILLPLYEVDDLRDAFRTLGLSGGSSGGSSGSETPGTSESA | |
| | TPESSGGSSGGDKKYSIGLAIGTNSVGWAVITDEYKVPSKKFKVLGNTDRHSIKKNLIG | |
| | ALLFDSGETAEATRLKRTARRRYTRRKNRICYLQEIFSNEMAKVDDSFFHRLEESFLVE | |
| | EDKKHERHPIFGNIVDEVAYHEKYPTIYHLRKKLVDSTDKADLRLIYLALAHMIKFRGH | |
| | FLIEGDLNPDNSDVDKLFIQLVQTYNQLFEENPINASGVDAKAILSARLSKSRRLENLI | |
| | AQLPGEKKNGLFGNLIALSLGLTPNFKSNFDLAEDAKLQLSKDTYDDDLDNLLAQIGDQ | |
| | YADLFLAAKNLSDAILLSDILRVNTEITKAPLSASMIKRYDEHHQDLTLLKALVRQQLP | |
| | EKYKEIFFDQSKNGYAGYIDGGASQEEFYKFIKPILEKMDGTEELLVKLNREDLLRKQR | |
| | TFDNGSIPHQIHLGELHAILRRQEDFYPFLKDNREKIEKILTFRIPYYVGPLARGNSRF | |
| | AWMTRKSEETITPWNFEEVVDKGASAQSFIERMTNFDKNLPNEKVLPKHSLLYEYFTVY | |
| | NELTKVKYVTEGMRKPAFLSGEQKKAIVDLLFKTNRKVTVKQLKEDYFKKIECFDSVEI | |
| | SGVEDRFNASLGTYHDLLKIIKDKDFLDNEENEDILEDIVLTLTLFEDREMIEERLKTY | |
| | AHLFDDKVMKQLKRRRYTGWGRLSRKLINGIRDKQSGKTILDFLKSDGFANRNFMQLIH | |
| | DDSLTFKEDIQKAQVSGQGDSLHEHIANLAGSPAIKKGILQTVKVVDELVKVMGRHKPE | |
| | NIVIEMARENQTTQKGQKNSRERMKRIEEGIKELGSQILKEHPVENTQLQNEKLYLYYL | |
| | QNGRDMYVDQELDINRLSDYDVDHIVPQSFLKDDSIDNKVLTRSDKNRGKSDNVPSEEV | |
| | VKKMKNYWRQLLNAKLITQRKFDNLTKAERGGLSELDKAGFIKRQLVETRQITKHVAQI | |
| | LDSRMNTKYDENDKLIREVKVITLKSKLVSDFRKDFQFYKVREINNYHHAHDAYLNAVV | |
| | GTALIKKYPKLESEFVYGDYKVYDVRKMIAKSEQEIGKATAKYFFYSNIMNFFKTEITL | |
| | ANGEIRKRPLIETNGETGEIVWDKGRDFATVRKVLSMPQVNIVKKTEVQTGGFSKESIL | |
| | PKRNSDKLIARKKDWDPKKYGGFDSPTVAYSVLVVAKVEKGKSKKLKSVKELLGITIME | |
| | RSSFEKNPIDFLEAKGYKEVKKDLIIKLPKYSLFELENGRKRMLASAGELQKGNELALP | |
| | SKYVNFLYLASHYEKLKGSPEDNEQKQLFVEQHKHYLDEIIEQISEFSKRVILADANLD | |
| | KVLSAYNKHRDKPIREQAENIIHLFTLTNLGAPAAFKYFDTTIDRKRYTSTKEVLDATL | |
| | IHQSITGLYETRIDLSQLGGD [symbol] [symbol] | |
| | [symbol] [symbol] [symbol] [symbol] | |
| | [symbol] [symbol] [symbol] | |
| | [symbol] [symbol] KRTADGSEFEPKKKRKV | |
| CDA-BE4 (or CDA1- | MKRTADGSEFESPKKKRKVTDAEYVRIHEKLDIYTFKKQFFNNKKSVSHRCYVLFELKR | 3203 |
| BE4max ) | RGERRACFWGYAVNKPQSGTERGIHAEIFSIRKVEEYLRDNPGQFTINWYSSWSPCADC | |
| Cas9 = SpCas9 | AEKILEWYNQELRGNGHTLKIWACKLYYEKNARNQIGLWNLRDNGVGLNVMVSEHYQCC | |
| | RKIFIQSSHNQLNENRWLEKTLKRAEKRRSELSIMIQVKILHTTKSPAVSGGSSGGSSG | |
| | SETPGTSESATPESSGGSSGGDKKYSIGLAIGTNSVGWAVITDEYKVPSKKFKVLGNTD | |
| | RHSIKKNLIGALLFDSGETAEATRLKRTARRRYTRRKNRICYLQEIFSNEMAKVDDSFF | |
| | HRLEESFLVEEDKKHERHPIFGNIVDEVAYHEKYPTIYHLRKKLVDSTDKADLRLIYLA | |
| | LAHMIKFRGHFLIEGDLNPDNSDVDKLFIQLVQTYNQLFEENPINASGVDAKAILSARL | |
| | SKSRRLENLIAQLPGEKKNGLFGNLIALSLGLTPNFKSNFDLAEDAKLQLSKDTYDDDL | |
| | DNLLAQIGDQYADLFLAAKNLSDAILLSDILRVNTEITKAPLSASMIKRYDEHHQDLTL | |
| | LKALVRQQLPEKYKEIFFDQSKNGYAGYIDGGASQEEFYKFIKPILEKMDGTEELLVKL | |
| | NREDLLRKQRTFDNGSIPHQIHLGELHAILRRQEDFYPFLKDNREKIEKILTFRIPYYV | |
| | GPLARGNSRFAWMTRKSEETITPWNFEEVVDKGASAQSFIERMTNFDKNLPNEKVLPKH | |
| | SLLYEYFTVYNELTKVKYVTEGMRKPAFLSGEQKKAIVDLLFKTNRKVTVKQLKEDYFK | |
| | KIECFDSVEISGVEDRFNASLGTYHDLLKIIKDKDFLDNEENEDILEDIVLTLTLFEDR | |
| | EMIEERLKTYAHLFDDKVMKQLKRRRYTGWGRLSRKLINGIRDKQSGKTILDFLKSDGF | |
| | ANRNFMQLIHDDSLTFKEDIQKAQVSGQGDSLHEHIANLAGSPAIKKGILQTVKVVDEL | |
| | VKVMGRHKPENIVIEMARENQTTQKGQKNSRERMKRIEEGIKELGSQILKEHPVENTQL | |
| | QNEKLYLYYLQNGRDMYVDQELDINRLSDYDVDHIVPQSFLKDDSIDNKVLTRSDKNRG | |
| | KSDNVPSEEVVKKMKNYWRQLLNAKLITQRKFDNLTKAERGGLSELDKAGFIKRQLVET | |
| | RQITKHVAQILDSRMNTKYDENDKLIREVKVITLKSKLVSDFRKDFQFYKVREINNYHH | |
| | AHDAYLNAVVGTALIKKYPKLESEFVYGDYKVYDVRKMIAKSEQEIGKATAKYFFYSNI | |
| | MNFFKTEITLANGEIRKRPLIETNGETGEIVWDKGRDFATVRKVLSMPQVNIVKKTEVQ | |
| | TGGFSKESILPKRNSDKLIARKKDWDPKKYGGFDSPTVAYSVLVVAKVEKGKSKKLKSV | |
| | KELLGITIMERSSFEKNPIDFLEAKGYKEVKKDLIIKLPKYSLFELENGRKRMLASAGE | |
| | LQKGNELALPSKYVNFLYLASHYEKLKGSPEDNEQKQLFVEQHKHYLDEIIEQISEFSK | |
| | RVILADANLDKVLSAYNKHRDKPIREQAENIIHLFTLTNLGAPAAFKYFDTTIDRKRYT | |
| | STKEVLDATLIHQSITGLYETRIDLSQLGGD [symbol] [symbol] | |
| | [symbol] [symbol] [symbol] [symbol] | |
| | [symbol] [symbol] [symbol] [symbol] | |
| | [symbol] [symbol] [symbol] | |
| | KRTADGSEFEPKKKRKV | |
| evoA-BE4 (or | MKRTADGSEFESPKKKRKVSKTGPVAVDPTLRRRIEPHEFEVFFDPRELRKETCLLYEI | 3204 |
| evoAPOBEC1-BE4max) | NWGGRHSIWRHTSQNTNKHVEVNFIEKFTTERYFCPNTRCSITWFLSWSPCGECSRAIT | |
| Cas9 = SpCas9 | EFLSRYPNVTLFIYIARLYHLANPRNRQGLRDLISSGVTIQIMTEQESGYCWHNFVNYS | |
| | PSNESHWPRYPHLWVRLYVLELYCIILGLPPCLNILRRKQSQLTSFTIALQSCHYQRLP | |
| | PHILWATGLKSGGSSGGSSGSETPGTSESATPESSGGSSGGDKKYSIGLAIGTNSVGWA | |
| | VITDEYKVPSKKFKVLGNTDRHSIKKNLIGALLFDSGETAEATRLKRTARRRYTRRKNR | |
| | ICYLQEIFSNEMAKVDDSFFHRLEESFLVEEDKKHERHPIFGNIVDEVAYHEKYPTIYH | |
| | LRKKLVDSTDKADLRLIYLALAHMIKFRGHFLIEGDLNPDNSDVDKLFIQLVQTYNQLF | |
| | EENPINASGVDAKAILSARLSKSRRLENLIAQLPGEKKNGLFGNLIALSLGLTPNFKSN | |
| | FDLAEDAKLQLSKDTYDDDLDNLLAQIGDQYADLFLAAKNLSDAILLSDILRVNTEITK | |
| | APLSASMIKRYDEHHQDLTLLKALVRQQLPEKYKEIFFDQSKNGYAGYIDGGASQEEFY | |
| | KFIKPILEKMDGTEELLVKLNREDLLRKQRTFDNGSIPHQIHLGELHAILRRQEDFYPF | |
| | LKDNREKIEKILTFRIPYYVGPLARGNSRFAWMTRKSEETITPWNFEEVVDKGASAQSF | |
| | IERMTNFDKNLPNEKVLPKHSLLYEYFTVYNELTKVKYVTEGMRKPAFLSGEQKKAIVD | |
| | LLFKTNRKVTVKQLKEDYFKKIECFDSVEISGVEDRFNASLGTYHDLLKIIKDKDFLDN | |
| | EENEDILEDIVLTLTLFEDREMIEERLKTYAHLFDDKVMKQLKRRRYTGWGRLSRKLIN | |
| | GIRDKQSGKTILDFLKSDGFANRNFMQLIHDDSLTFKEDIQKAQVSGQGDSLHEHIANL | |
| | AGSPAIKKGILQTVKVVDELVKVMGRHKPENIVIEMARENQTTQKGQKNSRERMKRIEE | |
| | GIKELGSQILKEHPVENTQLQNEKLYLYYLQNGRDMYVDQELDINRLSDYDVDHIVPQS | |
| | FLKDDSIDNKVLTRSDKNRGKSDNVPSEEVVKKMKNYWRQLLNAKLITQRKFDNLTKAE | |
| | RGGLSELDKAGFIKRQLVETRQITKHVAQILDSRMNTKYDENDKLIREVKVITLKSKLV | |
| | SDFRKDFQFYKVREINNYHHAHDAYLNAVVGTALIKKYPKLESEFVYGDYKVYDVRKMI | |
| | AKSEQEIGKATAKYFFYSNIMNFFKTEITLANGEIRKRPLIETNGETGEIVWDKGRDFA | |
| | TVRKVLSMPQVNIVKKTEVQTGGFSKESILPKRNSDKLIARKKDWDPKKYGGFDSPTVA | |
| | YSVLVVAKVEKGKSKKLKSVKELLGITIMERSSFEKNPIDFLEAKGYKEVKKDLIIKLP | |
| | KYSLFELENGRKRMLASAGELQKGNELALPSKYVNFLYLASHYEKLKGSPEDNEQKQLF | |
| | VEQHKHYLDEIIEQISEFSKRVILADANLDKVLSAYNKHRDKPIREQAENIIHLFTLTN | |
| | LGAPAAFKYFDTTIDRKRYTSTKEVLDATLIHQSITGLYETRIDLSQLGGD [symbol] | |
| | [symbol] [symbol] [symbol] [symbol] | |
| | [symbol] [symbol] [symbol] [symbol] [symbol] | |
| | [symbol] [symbol] [symbol] [symbol] [symbol] | |
| | [symbol] KRTADGSEFEPKKKRKV | |
| eA3A-BE4 | MKRTADGSEFESPKKKRKVEASPASGPRHLMDPHIFTSNFNNGIGRHKTYLCYEVERLD | 3205 |
| (or APOBEC3A) | NGTSVKMDQHRGFLHGQAKNLLCGFYGRHAELRFLDLVPSLQLDPAQIYRVTWFISWSP | |
| Cas9 = SpCas9 | CFSWGCAGEVRAFLQENTHVRLRIFAARIYDYDPLYKEALQMLRDAGAQVSIMTYDEFK | |
| | HCWDTFVDHQGCPFQPWDGLDEHSQALSGRLRAILQNQGNSGGSSGGSSGSETPGTSES | |
| | ATPESSGGSSGGDKKYSIGLAIGTNSVGWAVITDEYKVPSKKFKVLGNTDRHSIKKNLI | |
| | GALLFDSGETAEATRLKRTARRRYTRRKNRICYLQEIFSNEMAKVDDSFFHRLEESFLV | |
| | EEDKKHERHPIFGNIVDEVAYHEKYPTIYHLRKKLVDSTDKADLRLIYLALAHMIKFRG | |
| | HFLIEGDLNPDNSDVDKLFIQLVQTYNQLFEENPINASGVDAKAILSARLSKSRRLENL | |
| | IAQLPGEKKNGLFGNLIALSLGLTPNFKSNFDLAEDAKLQLSKDTYDDDLDNLLAQIGD | |
| | QYADLFLAAKNLSDAILLSDILRVNTEITKAPLSASMIKRYDEHHQDLTLLKALVRQQL | |
| | PEKYKEIFFDQSKNGYAGYIDGGASQEEFYKFIKPILEKMDGTEELLVKLNREDLLRKQ | |
| | RTFDNGSIPHQIHLGELHAILRRQEDFYPFLKDNREKIEKILTFRIPYYVGPLARGNSR | |
| | FAWMTRKSEETITPWNFEEVVDKGASAQSFIERMTNFDKNLPNEKVLPKHSLLYEYFTV | |
| | YNELTKVKYVTEGMRKPAFLSGEQKKAIVDLLFKTNRKVTVKQLKEDYFKKIECFDSVE | |
| | ISGVEDRFNASLGTYHDLLKIIKDKDFLDNEENEDILEDIVLTLTLFEDREMIEERLKT | |
| | YAHLFDDKVMKQLKRRRYTGWGRLSRKLINGIRDKQSGKTILDFLKSDGFANRNFMQLI | |
| | HDDSLTFKEDIQKAQVSGQGDSLHEHIANLAGSPAIKKGILQTVKVVDELVKVMGRHKP | |
| | ENIVIEMARENQTTQKGQKNSRERMKRIEEGIKELGSQILKEHPVENTQLQNEKLYLYY | |
| | LQNGRDMYVDQELDINRLSDYDVDHIVPQSFLKDDSIDNKVLTRSDKNRGKSDNVPSEE | |
| | VVKKMKNYWRQLLNAKLITQRKFDNLTKAERGGLSELDKAGFIKRQLVETRQITKHVAQ | |
| | ILDSRMNTKYDENDKLIREVKVITLKSKLVSDFRKDFQFYKVREINNYHHAHDAYLNAV | |
| | VGTALIKKYPKLESEFVYGDYKVYDVRKMIAKSEQEIGKATAKYFFYSNIMNFFKTEIT | |
| | LANGEIRKRPLIETNGETGEIVWDKGRDFATVRKVLSMPQVNIVKKTEVQTGGFSKESI | |
| | LPKRNSDKLIARKKDWDPKKYGGFDSPTVAYSVLVVAKVEKGKSKKLKSVKELLGITIM | |
| | ERSSFEKNPIDFLEAKGYKEVKKDLIIKLPKYSLFELENGRKRMLASAGELQKGNELAL | |
| | PSKYVNFLYLASHYEKLKGSPEDNEQKQLFVEQHKHYLDEIIEQISEFSKRVILADANL | |
| | DKVLSAYNKHRDKPIREQAENIIHLFTLTNLGAPAAFKYFDTTIDRKRYTSTKEVLDAT | |
| | LIHQSITGLYETRIDLSQLGGD [symbol] | |
| | [symbol] [symbol] | |
| | [symbol] [symbol] | |
| | [symbol] KRTADGSEFEPKKKRKV | |
| eA3A-T31A | MKRTADGSEFESPKKKRKVEASPASGPRHLMDPHIFTSNFNNGIGRHKAYLCYEVERLD | 3206 |
| Cas9 = SpCas9 | NGTSVKMDQHRGFLHGQAKNLLCGFYGRHAELRFLDLVPSLQLDPAQIYRVTWFISWSP | |
| | CFSWGCAGEVRAFLQENTHVRLRIFAARIYDYDPLYKEALQMLRDAGAQVSIMTYDEFK | |
| | HCWDTFVDHQGCPFQPWDGLDEHSQALSGRLRAILQNQGNSGGSSGGSSGSETPGTSES | |
| | ATPESSGGSSGGDKKYSIGLAIGTNSVGWAVITDEYKVPSKKFKVLGNTDRHSIKKNLI | |
| | GALLFDSGETAEATRLKRTARRRYTRRKNRICYLQEIFSNEMAKVDDSFFHRLEESFLV | |
| | EEDKKHERHPIFGNIVDEVAYHEKYPTIYHLRKKLVDSTDKADLRLIYLALAHMIKFRG | |
| | HFLIEGDLNPDNSDVDKLFIQLVQTYNQLFEENPINASGVDAKAILSARLSKSRRLENL | |
| | IAQLPGEKKNGLFGNLIALSLGLTPNFKSNFDLAEDAKLQLSKDTYDDDLDNLLAQIGD | |
| | QYADLFLAAKNLSDAILLSDILRVNTEITKAPLSASMIKRYDEHHQDLTLLKALVRQQL | |
| | PEKYKEIFFDQSKNGYAGYIDGGASQEEFYKFIKPILEKMDGTEELLVKLNREDLLRKQ | |
| | RTFDNGSIPHQIHLGELHAILRRQEDFYPFLKDNREKIEKILTFRIPYYVGPLARGNSR | |
| | FAWMTRKSEETITPWNFEEVVDKGASAQSFIERMTNFDKNLPNEKVLPKHSLLYEYFTV | |
| | YNELTKVKYVTEGMRKPAFLSGEQKKAIVDLLFKTNRKVTVKQLKEDYFKKIECFDSVE | |
| | ISGVEDRFNASLGTYHDLLKIIKDKDFLDNEENEDILEDIVLTLTLFEDREMIEERLKT | |
| | YAHLFDDKVMKQLKRRRYTGWGRLSRKLINGIRDKQSGKTILDFLKSDGFANRNFMQLI | |
| | HDDSLTFKEDIQKAQVSGQGDSLHEHIANLAGSPAIKKGILQTVKVVDELVKVMGRHKP | |
| | ENIVIEMARENQTTQKGQKNSRERMKRIEEGIKELGSQILKEHPVENTQLQNEKLYLYY | |
| | LQNGRDMYVDQELDINRLSDYDVDHIVPQSFLKDDSIDNKVLTRSDKNRGKSDNVPSEE | |
| | VVKKMKNYWRQLLNAKLITQRKFDNLTKAERGGLSELDKAGFIKRQLVETRQITKHVAQ | |
| | ILDSRMNTKYDENDKLIREVKVITLKSKLVSDFRKDFQFYKVREINNYHHAHDAYLNAV | |
| | VGTALIKKYPKLESEFVYGDYKVYDVRKMIAKSEQEIGKATAKYFFYSNIMNFFKTEIT | |
| | LANGEIRKRPLIETNGETGEIVWDKGRDFATVRKVLSMPQVNIVKKTEVQTGGFSKESI | |
| | LPKRNSDKLIARKKDWDPKKYGGFDSPTVAYSVLVVAKVEKGKSKKLKSVKELLGITIM | |
| | ERSSFEKNPIDFLEAKGYKEVKKDLIIKLPKYSLFELENGRKRMLASAGELQKGNELAL | |
| | PSKYVNFLYLASHYEKLKGSPEDNEQKQLFVEQHKHYLDEIIEQISEFSKRVILADANL | |
| | DKVLSAYNKHRDKPIREQAENIIHLFTLTNLGAPAAFKYFDTTIDRKRYTSTKEVLDAT | |
| | LIHQSITGLYETRIDLSQLGGD [symbol] [symbol] | |
| | [symbol] [symbol] [symbol] | |
| | [symbol] [symbol] [symbol] [symbol] | |
| | [symbol] [symbol] [symbol] KRTADGSEFEPKKKRKV | |
| eA3A-BE5 | MKRTADGSEFESPKKKRKVEASPASGPRHLMDPHIFTSNFNNGIGRHKTYLCYEVERLD | 3207 |
| Cas9 = SpCas9 | NGDAVKMDQHRGFLHGQAKNLLCGFYGRHAELRFLDLVPSLQLDPAQIYRVTWFISWSP | |
| | CFSWGCAGEVRAFLQENTHVRLRIFAARIYDYDPLYKEALQMLRDAGAQVSIMTYDEFK | |
| | HCWDTFVDHQGCPFQPWDGLDEHSQALSGRLRAILQNQGNSGGSSGGSSGSETPGTSES | |
| | ATPESSGGSSGGDKKYSIGLAIGTNSVGWAVITDEYKVPSKKFKVLGNTDRHSIKKNLI | |
| | GALLFDSGETAEATRLKRTARRRYTRRKNRICYLQEIFSNEMAKVDDSFFHRLEESFLV | |
| | EEDKKHERHPIFGNIVDEVAYHEKYPTIYHLRKKLVDSTDKADLRLIYLALAHMIKFRG | |
| | HFLIEGDLNPDNSDVDKLFIQLVQTYNQLFEENPINASGVDAKAILSARLSKSRRLENL | |
| | IAQLPGEKKNGLFGNLIALSLGLTPNFKSNFDLAEDAKLQLSKDTYDDDLDNLLAQIGD | |
| | QYADLFLAAKNLSDAILLSDILRVNTEITKAPLSASMIKRYDEHHQDLTLLKALVRQQL | |
| | PEKYKEIFFDQSKNGYAGYIDGGASQEEFYKFIKPILEKMDGTEELLVKLNREDLLRKQ | |
| | RTFDNGSIPHQIHLGELHAILRRQEDFYPFLKDNREKIEKILTFRIPYYVGPLARGNSR | |
| | FAWMTRKSEETITPWNFEEVVDKGASAQSFIERMTNFDKNLPNEKVLPKHSLLYEYFTV | |
| | YNELTKVKYVTEGMRKPAFLSGEQKKAIVDLLFKTNRKVTVKQLKEDYFKKIECFDSVE | |
| | ISGVEDRFNASLGTYHDLLKIIKDKDFLDNEENEDILEDIVLTLTLFEDREMIEERLKT | |
| | YAHLFDDKVMKQLKRRRYTGWGRLSRKLINGIRDKQSGKTILDFLKSDGFANRNFMQLI | |
| | HDDSLTFKEDIQKAQVSGQGDSLHEHIANLAGSPAIKKGILQTVKVVDELVKVMGRHKP | |
| | ENIVIEMARENQTTQKGQKNSRERMKRIEEGIKELGSQILKEHPVENTQLQNEKLYLYY | |
| | LQNGRDMYVDQELDINRLSDYDVDHIVPQSFLKDDSIDNKVLTRSDKNRGKSDNVPSEE | |
| | VVKKMKNYWRQLLNAKLITQRKFDNLTKAERGGLSELDKAGFIKRQLVETRQITKHVAQ | |
| | ILDSRMNTKYDENDKLIREVKVITLKSKLVSDFRKDFQFYKVREINNYHHAHDAYLNAV | |
| | VGTALIKKYPKLESEFVYGDYKVYDVRKMIAKSEQEIGKATAKYFFYSNIMNFFKTEIT | |
| | LANGEIRKRPLIETNGETGEIVWDKGRDFATVRKVLSMPQVNIVKKTEVQTGGFSKESI | |
| | LPKRNSDKLIARKKDWDPKKYGGFDSPTVAYSVLVVAKVEKGKSKKLKSVKELLGITIM | |
| | ERSSFEKNPIDFLEAKGYKEVKKDLIIKLPKYSLFELENGRKRMLASAGELQKGNELAL | |
| | PSKYVNFLYLASHYEKLKGSPEDNEQKQLFVEQHKHYLDEIIEQISEFSKRVILADANL | |
| | DKVLSAYNKHRDKPIREQAENIIHLFTLTNLGAPAAFKYFDTTIDRKRYTSTKEVLDAT | |
| | LIHQSITGLYETRIDLSQLGGD [symbol] [symbol] | |
| | [symbol] [symbol] [symbol] [symbol] | |
| | [symbol] [symbol] | |
| | [symbol] [symbol] | |
| | KRTADGSEFEPKKKRKV | |
| BE4-CP1028 | MKRTADGSEFESPKKKRKVSSETGPVAVDPTLRRRIEPHEFEVFFDPRELRKETCLLYE | 3208 |
| Cas9 = Cas9 CP1028 | INWGGRHSIWRHTSQNTNKHVEVNFIEKFTTERYFCPNTRCSITWFLSWSPCGECSRAI | |
| | TEFLSRYPHVTLFIYIARLYHHADPRNRQGLRDLISSGVTIQIMTEQESGYCWRNFVNY | |
| | SPSNEAHWPRYPHLWVRLYVLELYCIILGLPPCLNILRRKQPQLTFFTIALQSCHYQRL | |
| | PPHILWATGLKSGGSSGGSSGSETPGTSESATPESSGGSSGGEIGKATAKYFFYSNIMN | |
| | FFKTEITLANGEIRKRPLIETNGETGEIVWDKGRDFATVRKVLSMPQVNIVKKTEVQTG | |
| | GFSKESILPKRNSDKLIARKKDWDPKKYGGFDSPTVAYSVLVVAKVEKGKSKKLKSVKE | |
| | LLGITIMERSSFEKNPIDFLEAKGYKEVKKDLIIKLPKYSLFELENGRKRMLASAGELQ | |
| | KGNELALPSKYVNFLYLASHYEKLKGSPEDNEQKQLFVEQHKHYLDEIIEQISEFSKRV | |
| | ILADANLDKVLSAYNKHRDKPIREQAENIIHLFTLTNLGAPAAFKYFDTTIDRKRYTST | |
| | KEVLDATLIHQSITGLYETRIDLSQLGGDGGSGGSGGSGGSGGSGGSGGMDKKYSIGLA | |
| | IGTNSVGWAVITDEYKVPSKKFKVLGNTDRHSIKKNLIGALLFDSGETAEATRLKRTAR | |
| | RRYTRRKNRICYLQEIFSNEMAKVDDSFFHRLEESFLVEEDKKHERHPIFGNIVDEVAY | |
| | HEKYPTIYHLRKKLVDSTDKADLRLIYLALAHMIKFRGHFLIEGDLNPDNSDVDKLFIQ | |
| | LVQTYNQLFEENPINASGVDAKAILSARLSKSRRLENLIAQLPGEKKNGLFGNLIALSL | |
| | GLTPNFKSNFDLAEDAKLQLSKDTYDDDLDNLLAQIGDQYADLFLAAKNLSDAILLSDI | |
| | LRVNTEITKAPLSASMIKRYDEHHQDLTLLKALVRQQLPEKYKEIFFDQSKNGYAGYID | |
| | GGASQEEFYKFIKPILEKMDGTEELLVKLNREDLLRKQRTFDNGSIPHQIHLGELHAIL | |
| | RRQEDFYPFLKDNREKIEKILTFRIPYYVGPLARGNSRFAWMTRKSEETITPWNFEEVV | |
| | DKGASAQSFIERMTNFDKNLPNEKVLPKHSLLYEYFTVYNELTKVKYVTEGMRKPAFLS | |
| | GEQKKAIVDLLFKTNRKVTVKQLKEDYFKKIECFDSVEISGVEDRFNASLGTYHDLLKI | |
| | IKDKDFLDNEENEDILEDIVLTLTLFEDREMIEERLKTYAHLFDDKVMKQLKRRRYTGW | |
| | GRLSRKLINGIRDKQSGKTILDFLKSDGFANRNFMQLIHDDSLTFKEDIQKAQVSGQGD | |
| | SLHEHIANLAGSPAIKKGILQTVKVVDELVKVMGRHKPENIVIEMARENQTTQKGQKNS | |
| | RERMKRIEEGIKELGSQILKEHPVENTQLQNEKLYLYYLQNGRDMYVDQELDINRLSDY | |
| | DVDHIVPQSFLKDDSIDNKVLTRSDKNRGKSDNVPSEEVVKKMKNYWRQLLNAKLITQR | |
| | KFDNLTKAERGGLSELDKAGFIKRQLVETRQITKHVAQILDSRMNTKYDENDKLIREVK | |
| | VITLKSKLVSDFRKDFQFYKVREINNYHHAHDAYLNAVVGTALIKKYPKLESEFVYGDY | |
| | KVYDVRKMIAKSEQ [symbol] [symbol] [symbol] | |
| | [symbol] [symbol] [symbol] [symbol] | |
| | [symbol] [symbol] [symbol] [symbol] | |
| | [symbol] [symbol] [symbol] KRTADGSEFEPKKKRKV | |
| BE4-Cas9-NG | MKRTADGSEFESPKKKRKVSSETGPVAVDPTLRRRIEPHEFEVFFDPRELRKETCLLYE | 3209 |
| Cas9 = Cas9 NG | INWGGRHSIWRHTSQNTNKHVEVNFIEKFTTERYFCPNTRCSITWFLSWSPCGECSRAI | |
| | TEFLSRYPHVTLFIYIARLYHHADPRNRQGLRDLISSGVTIQIMTEQESGYCWRNFVNY | |
| | SPSNEAHWPRYPHLWVRLYVLELYCIILGLPPCLNILRRKQPQLTFFTIALQSCHYQRL | |
| | PPHILWATGLKSGGSSGGSSGSETPGTSESATPESSGGSSGGDKKYSIGLAIGTNSVGW | |
| | AVITDEYKVPSKKFKVLGNTDRHSIKKNLIGALLFDSGETAEATRLKRTARRRYTRRKN | |
| | RICYLQEIFSNEMAKVDDSFFHRLEESFLVEEDKKHERHPIFGNIVDEVAYHEKYPTIY | |
| | HLRKKLVDSTDKADLRLIYLALAHMIKFRGHFLIEGDLNPDNSDVDKLFIQLVQTYNQL | |
| | FEENPINASGVDAKAILSARLSKSRRLENLIAQLPGEKKNGLFGNLIALSLGLTPNFKS | |
| | NFDLAEDAKLQLSKDTYDDDLDNLLAQIGDQYADLFLAAKNLSDAILLSDILRVNTEIT | |
| | KAPLSASMIKRYDEHHQDLTLLKALVRQQLPEKYKEIFFDQSKNGYAGYIDGGASQEEF | |
| | YKFIKPILEKMDGTEELLVKLNREDLLRKQRTFDNGSIPHQIHLGELHAILRRQEDFYP | |
| | FLKDNREKIEKILTFRIPYYVGPLARGNSRFAWMTRKSEETITPWNFEEVVDKGASAQS | |
| | FIERMTNFDKNLPNEKVLPKHSLLYEYFTVYNELTKVKYVTEGMRKPAFLSGEQKKAIV | |
| | DLLFKTNRKVTVKQLKEDYFKKIECFDSVEISGVEDRFNASLGTYHDLLKIIKDKDFLD | |
| | NEENEDILEDIVLTLTLFEDREMIEERLKTYAHLFDDKVMKQLKRRRYTGWGRLSRKLI | |
| | NGIRDKQSGKTILDFLKSDGFANRNFMQLIHDDSLTFKEDIQKAQVSGQGDSLHEHIAN | |
| | LAGSPAIKKGILQTVKVVDELVKVMGRHKPENIVIEMARENQTTQKGQKNSRERMKRIE | |
| | EGIKELGSQILKEHPVENTQLQNEKLYLYYLQNGRDMYVDQELDINRLSDYDVDHIVPQ | |
| | SFLKDDSIDNKVLTRSDKNRGKSDNVPSEEVVKKMKNYWRQLLNAKLITQRKFDNLTKA | |
| | ERGGLSELDKAGFIKRQLVETRQITKHVAQILDSRMNTKYDENDKLIREVKVITLKSKL | |
| | VSDFRKDFQFYKVREINNYHHAHDAYLNAVVGTALIKKYPKLESEFVYGDYKVYDVRKM | |
| | IAKSEQEIGKATAKYFFYSNIMNFFKTEITLANGEIRKRPLIETNGETGEIVWDKGRDF | |
| | ATVRKVLSMPQVNIVKKTEVQTGGFSKESIRPKRNSDKLIARKKDWDPKKYGGFVSPTV | |
| | AYSVLVVAKVEKGKSKKLKSVKELLGITIMERSSFEKNPIDFLEAKGYKEVKKDLIIKL | |
| | PKYSLFELENGRKRMLASARFLQKGNELALPSKYVNFLYLASHYEKLKGSPEDNEQKQL | |
| | FVEQHKHYLDEIIEQISEFSKRVILADANLDKVLSAYNKHRDKPIREQAENIIHLFTLT | |
| | NLGAPRAFKYFDTTIDRKVYRSTKEVLDATLIHQSITGLYETRIDLSQLGGD [symbol] | |
| | [symbol] [symbol] [symbol] [symbol] | |
| | [symbol] [symbol] [symbol] [symbol] | |
| | [symbol] [symbol] [symbol] [symbol] | |
| | [symbol] KRTADGSEFEPKKKRKV | |

The following ABEs were used to generate training data for the BE-Hive algorithm of Example 1.
Each of the ABEs have the same architecture of [NLS]-[deaminase]-[Cas9]-[NLS] (which is the
ABEmax architecture) and use the same adenine deaminase, ABE7.10, with either the SpCas9 or
CP1041 circular permutant variant as the Cas9 component.

| In further detail, the architecture of ABEmax is: | | |
|---|---|---|
| [bpNLS]-[wt TadA]-[evolved TadA*]-[Cas9 D10A]-[bpNLS] | | |
| Key: | | |
| NLS (N-terminal) | Single underline | |
| ABE7.10 | Double underline | |
| Linker | Italic | |
| SpCas9 | Plain | |
| Linker + 2xUGI | Bold underline | |
| NLS (C-terminal) | Single underline + italic | |
| ABEmax (or ABE) | MKRTADGSEFESPKKKRKVSEVEFSHEYWMRHALTLAKRAWDEREVPVGAVLVHNNRVI | 3210 |
| | GEGWNRPIGRHDPTAHAEIMALRQGGLVMQNYRLIDATLYVTLEPCVMCAGAMIHSRIG | |
| | RVVFGARDAKTGAAGSLMDVLHHPGMNHRVEITEGILADECAALLSDFFRMRRQEIKAQ | |
| | KKAQSSTDSGGSSGGSSGSETPGTSESATPESSGGSSGGSSEVEFSHEYWMRHALTLAK | |
| | RARDEREVPVGAVLVLNNRVIGEGWNRAIGLHDPTAHAEIMALRQGGLVMQNYRLIDAT | |
| | LYVTFEPCVMCAGAMIHSRIGRVVFGVRNAKTGAAGSLMDVLHYPGMNHRVEITEGILA | |
| | DECAALLCYFFRMPRQVFNAQKKAQSSTDSGGSSGGSSGSETPGTSESATPESSGGSSG | |
| | GSDKKYSIGLAIGTNSVGWAVITDEYKVPSKKFKVLGNTDRHSIKKNLIGALLFDSGET | |
| | AEATRLKRTARRRYTRRKNRICYLQEIFSNEMAKVDDSFFHRLEESFLVEEDKKHERHP | |
| | IFGNIVDEVAYHEKYPTIYHLRKKLVDSTDKADLRLIYLALAHMIKFRGHFLIEGDLNP | |
| | DNSDVDKLFIQLVQTYNQLFEENPINASGVDAKAILSARLSKSRRLENLIAQLPGEKKN | |
| | GLFGNLIALSLGLTPNFKSNFDLAEDAKLQLSKDTYDDDLDNLLAQIGDQYADLFLAAK | |
| | NLSDAILLSDILRVNTEITKAPLSASMIKRYDEHHQDLTLLKALVRQQLPEKYKEIFFD | |
| | QSKNGYAGYIDGGASQEEFYKFIKPILEKMDGTEELLVKLNREDLLRKQRTFDNGSIPH | |
| | QIHLGELHAILRRQEDFYPFLKDNREKIEKILTFRIPYYVGPLARGNSRFAWMTRKSEE | |
| | TITPWNFEEVVDKGASAQSFIERMTNFDKNLPNEKVLPKHSLLYEYFTVYNELTKVKYV | |
| | TEGMRKPAFLSGEQKKAIVDLLFKTNRKVTVKQLKEDYFKKIECFDSVEISGVEDRFNA | |
| | SLGTYHDLLKIIKDKDFLDNEENEDILEDIVLTLTLFEDREMIEERLKTYAHLFDDKVM | |
| | KQLKRRRYTGWGRLSRKLINGIRDKQSGKTILDFLKSDGFANRNFMQLIHDDSLTFKED | |
| | IQKAQVSGQGDSLHEHIANLAGSPAIKKGILQTVKVVDELVKVMGRHKPENIVIEMARE | |
| | NQTTQKGQKNSRERMKRIEEGIKELGSQILKEHPVENTQLQNEKLYLYYLQNGRDMYVD | |
| | QELDINRLSDYDVDHIVPQSFLKDDSIDNKVLTRSDKNRGKSDNVPSEEVVKKMKNYWR | |
| | QLLNAKLITQRKFDNLTKAERGGLSELDKAGFIKRQLVETRQITKHVAQILDSRMNTKY | |
| | DENDKLIREVKVITLKSKLVSDFRKDFQFYKVREINNYHHAHDAYLNAVVGTALIKKYP | |
| | KLESEFVYGDYKVYDVRKMIAKSEQEIGKATAKYFFYSNIMNFFKTEITLANGEIRKRP | |
| | LIETNGETGEIVWDKGRDFATVRKVLSMPQVNIVKKTEVQTGGFSKESILPKRNSDKLI | |
| | ARKKDWDPKKYGGFDSPTVAYSVLVVAKVEKGKSKKLKSVKELLGITIMERSSFEKNPI | |
| | DFLEAKGYKEVKKDLIIKLPKYSLFELENGRKRMLASAGELQKGNELALPSKYVNFLYL | |
| | ASHYEKLKGSPEDNEQKQLFVEQHKHYLDEIIEQISEFSKRVILADANLDKVLSAYNKH | |
| | RDKPIREQAENIIHLFTLTNLGAPAAFKYFDTTIDRKRYTSTKEVLDATLIHQSITGLY | |
| | ETRIDLSQLGGD [symbol] [symbol] [symbol] [symbol] | |
| | [symbol] [symbol] [symbol] [symbol] | |
| | [symbol] [symbol] [symbol] | |
| | [symbol] [symbol] KRTADGSEFEPKKKRKV | |
| ABE-CP1041 (or ABE-CP) | MKRTADGSEFESPKKKRKVSEVEFSHEYWMRHALTLAKRAWDEREVPVGAVLVHNNRVI | 3211 |
| | GEGWNRPIGRHDPTAHAEIMALRQGGLVMQNYRLIDATLYVTLEPCVMCAGAMIHSRIG | |
| | RWFGARDAKTGAAGSLMDVLHHPGMNHRVEITEGILADECAALLSDFFRMRRQEIKAQ | |
| | KKAQSSTDSGGSSGGSSGSETPGTSESATPESSGGSSGGSSEVEFSHEYWMRHALTLAK | |
| | RARDEREVPVGAVLVLNNRVIGEGWNRAIGLHDPTAHAEIMALRQGGLVMQNYRLIDAT | |
| | LYVTFEPCVMCAGAMIHSRIGRVVFGVRNAKTGAAGSLMDVLHYPGMNHRVEITEGILA | |
| | DECAALLCYFFRMPRQVFNAQKKAQSSTDSGGSSGGSSGSETPGTSESATPESSGGSSG | |
| | GSNIMNFFKTEITLANGEIRKRPLIETNGETGEIVWDKGRDFATVRKVLSMPQVNIVKK | |
| | TEVQTGGFSKESILPKRNSDKLIARKKDWDPKKYGGFDSPTVAYSVLVVAKVEKGKSKK | |
| | LKSVKELLGITIMERSSFEKNPIDFLEAKGYKEVKKDLIIKLPKYSLFELENGRKRMLA | |
| | SAGELQKGNELALPSKYVNFLYLASHYEKLKGSPEDNEQKQLFVEQHKHYLDEIIEQIS | |
| | EFSKRVILADANLDKVLSAYNKHRDKPIREQAENIIHLFTLTNLGAPAAFKYFDTTIDR | |
| | KRYTSTKEVLDATLIHQSITGLYETRIDLSQLGGDGGSGGSGGSGGSGGSGGSGGDKKY | |
| | SIGLAIGTNSVGWAVITDEYKVPSKKFKVLGNTDRHSIKKNLIGALLFDSGETAEATRL | |
| | KRTARRRYTRRKNRICYLQEIFSNEMAKVDDSFFHRLEESFLVEEDKKHERHPIFGNIV | |
| | DEVAYHEKYPTIYHLRKKLVDSTDKADLRLIYLALAHMIKFRGHFLIEGDLNPDNSDVD | |
| | KLFIQLVQTYNQLFEENPINASGVDAKAILSARLSKSRRLENLIAQLPGEKKNGLFGNL | |
| | IALSLGLTPNFKSNFDLAEDAKLQLSKDTYDDDLDNLLAQIGDQYADLFLAAKNLSDAI | |
| | LLSDILRVNTEITKAPLSASMIKRYDEHHQDLTLLKALVRQQLPEKYKEIFFDQSKNGY | |
| | AGYIDGGASQEEFYKFIKPILEKMDGTEELLVKLNREDLLRKQRTFDNGSIPHQIHLGE | |
| | LHAILRRQEDFYPFLKDNREKIEKILTFRIPYYVGPLARGNSRFAWMTRKSEETITPWN | |
| | FEEVVDKGASAQSFIERMTNFDKNLPNEKVLPKHSLLYEYFTVYNELTKVKYVTEGMRK | |
| | PAFLSGEQKKAIVDLLFKTNRKVTVKQLKEDYFKKIECFDSVEISGVEDRFNASLGTYH | |
| | DLLKIIKDKDFLDNEENEDILEDIVLTLTLFEDREMIEERLKTYAHLFDDKVMKQLKRR | |
| | RYTGWGRLSRKLINGIRDKQSGKTILDFLKSDGFANRNFMQLIHDDSLTFKEDIQKAQV | |
| | SGQGDSLHEHIANLAGSPAIKKGILQTVKVVDELVKVMGRHKPENIVIEMARENQTTQK | |
| | GQKNSRERMKRIEEGIKELGSQILKEHPVENTQLQNEKLYLYYLQNGRDMYVDQELDIN | |
| | RLSDYDVDHIVPQSFLKDDSIDNKVLTRSDKNRGKSDNVPSEEVVKKMKNYWRQLLNAK | |
| | LITQRKFDNLTKAERGGLSELDKAGFIKRQLVETRQITKHVAQILDSRMNTKYDENDKL | |
| | IREVKVITLKSKLVSDFRKDFQFYKVREINNYHHAHDAYLNAVVGTALIKKYPKLESEF | |
| | VYGDYKVYDVRKMIAKSEQEIGKATAKYFFY [symbol] [symbol] | |
| | [symbol] [symbol] [symbol] [symbol] | |
| | [symbol] [symbol] [symbol] [symbol] | |
| | [symbol] [symbol] [symbol] [symbol] | |
| | KRTADGSEFEPKKKRKV | |

The above base editors used in generating training data for Example 1 can be further found described in (a) Koblan, L. W., Doman, J. L., Wilson, C., Levy, J. M., Tay, T., Newby, G. A., Maianti, J. P., Raguram, A., and Liu, D. R. (2018). Improving cytidine and adenine base editors by expression optimization and ancestral reconstruction. Nat. Biotechnol. 36, 843-848; (b) Gehrke, J. M., Cervantes, O., Clement, M. K., Wu, Y., Zeng, J., Bauer, D. E., Pinello, L., and Joung, J. K. (2018). An APOBeC3A-Cas9 base editor with minimized bystander and off-target activities. Nat. Biotechnol. 36, 977; (c) Huang, T. P., Zhao, K. T., Miller, S. M., Gaudelli, N. M., Oakes, B. L., Fellmann, C., Savage, D. F., and Liu, D. R. (2019). Circularly permuted and PAM-modified Cas9 variants broaden the targeting scope of base editors. Nat. Biotechnol; (d) Komor, A. C., Zhao, K. T., Packer, M. S., Gaudelli, N. M., Waterbury, A. L., Koblan, L. W., Kim, Y. B., Badran, A. H., and Liu, D. R. (2017). Improved base excision repair inhibition and bacteriophage Mu Gam protein yields C:G-to-T:A base editors with higher efficiency and product purity. Sci. Adv. 3, eaao4774; (e) Thuronyi, B. W., Koblan, L. W., Levy, J. M., Yeh, W.-H., Zheng, C., Newby, G. A., Wilson, C., Bhaumik, M., Shubina-Oleinik, O., Holt, J. R., et al. (2019). Continuous evolution of base editors with expanded target compatibility and improved activity. Nat. Biotechnol.; and (f) Gaudelli, N. M., Komor, A. C., Rees, H. A., Packer, M. S., Badran, A. H., Bryson, D. I., and Liu, D. R. (2017). Programmable base editing of A⋅T to G⋅C in genomic DNA without DNA cleavage. Nature 551, 464-471, each of which are incorporated herein by reference.

### EXAMPLES

### Example 1. Target Sequence and Deaminase Determinants of Base Editing Outcomes Revealed by Substrate Library Analysis and Machine Learning (BE-Hive Algorithm)

### Summary

Base editors are widely used tools that enable targeted point mutations in DNA, but the factors that determine base editing outcomes have not been comprehensively studied, impeding the optimal choice and use of base editors from among many reported variants. The sequence activity relationships of 11 cytosine and adenine base editors (CBEs and ABEs) on 38,538 genomically-integrated targets in mammalian cells were characterized, and the resulting outcomes were used to develop BE-Hive (crisprbehive.design), a machine learning model that predicts base editing genotypes (R≈0.9) and efficiency (R≈0.7). The genotypes of 3,388 disease-associated SNVs were corrected with ≥90% precision consistent with prediction (R=0.78-0.92), including 675 alleles with bystander nucleotides that BE-Hive correctly predicted would not be efficiently edited. Sequence determinants of previously unpredictable CBE-mediated transversions were discovered and corrected 174 SNVs with >90% precision among edited amino acid sequences by C⋅G-to-G⋅C and C⋅G-to-A⋅T editing. Base editing outcomes were also discovered that were not predicted by inspection, but could be accurately captured and predicted by BE-Hive. Finally, a new role was established for CBE-deaminases in resolving U⋅G intermediates was established, and base editor variants that modulated this process were engineered. These discoveries deepen the understanding of base editors, enable their use at previously intractable targets, and provide new base editors with improved editing capabilities.

### Introduction

Programmable editing of single nucleotides in genomic DNA is a key capability for both research and therapeutic applications (Adli, 2018; Anzalone et al., 2019; Doench et al., 2016; Doudna and Knott, 2018; Pérez-Palma et al., 2019; Rees and Liu, 2018; Shen et al., 2018). Single-nucleotide variants (SNVs) represent approximately half of known pathogenic alleles (Landrum et al., 2016; Stenson et al., 2014), and thus targeted installation of point mutations can facilitate the study or potential treatment of genetic disorders. Previously, cytosine deaminases were developed, and laboratory-evolved adenine deaminase enzymes fused to catalytically impaired CRISPR-Cas proteins to enable cytosine and adenine base editing in living cells in a programmable fashion without requiring a DNA double-strand break or a donor DNA template (Gaudelli et al., 2017; Gehrke et al., 2018; Huang et al., 2019; Komor et al., 2016; Nishida et al., 2016; Thuronyi et al., 2019; Yeh et al., 2018). Cytosine base editors (CBEs) and adenine base editors (ABEs) together enable all four transition point mutations (C→T, T→C, A→G, and G→A) and routinely achieve high ratios of desired sequence substitutions relative to undesired insertions and deletions (indels) (Lin et al., 2014; Paquet et al., 2016). Base editing has been applied in a wide range of organisms ranging from bacteria to plants to primates (Rees and Liu, 2018), and has already been used to correct pathogenic mutations in animal models, in some cases with phenotypic rescue (Chadwick et al., 2017; Liang et al., 2017; Min et al., 2019; Ryu et al., 2018; Song et al., 2019; Villiger et al., 2018; Yeh et al., 2018; Zeng et al., 2018), establishing its potential for clinical applications.

The utility of base editing has inspired the development of many cytosine and adenine base editor variants with distinct editing properties (Adli, 2018; Molla and Yang, 2019; Rees and Liu, 2018). To date, these properties have been gleaned by analyzing base editing outcomes at a modest number of genomic sites, often chosen to align with previous genome editing studies (Gaudelli et al., 2017; Gehrke et al., 2018; Huang et al., 2019; Komor et al., 2016; Thuronyi et al., 2019). The interplay between base editor and target sequence, however, influences base editing outcomes in complex and occasionally unintuitive ways (Gehrke et al., 2018; Huang et al., 2019; Tan et al., 2019; Thuronyi et al., 2019; Villiger et al., 2018). As a result, obtaining a desired genotype with useful efficiencies often requires empirical optimization of base editor and single guide RNA (sgRNA) choice for each target. Likewise, some viable targets that do not fit canonical guidelines for base editing use may be overlooked since simple guidelines for target selection likely do not fully capture the scope of base editing.

A systematic and comprehensive analysis of sequence and deaminase determinants of base editing thus would enhance the understanding of base editors, facilitate their use in precision editing applications, and guide development of new base editors with enhanced abilities to induce or prevent rare base editing outcomes. As described herein, libraries of 38,538 total pairs of sgRNAs and target sequences were developed and integrated into three mammalian cell types to comprehensively characterize base editing outcomes and sequence-activity relationships for eight popular cytosine and adenine base editors in living cells. The roles of deaminases, sequence context, and cell type in determining genotypes that result from base editing were analyzed, and a machine learning model was developed that accurately predicts base editing outcomes, including many previously unpredictable features, at any target site of interest. Using the resulting information, a variety of base editors were applied, including newly engineered variants, to precisely correct 3,388 genotypes and 2,399 coding sequences of disease-associated SNVs to wild-type with ≥90% precision among edited products, including by previously poorly understood non-canonical base editing outcomes. These findings substantially extend the understanding of base editing and reveal new capabilities of both new and previously described base editors.

### Results

### Development of a Genome-Integrated Target Site Library Assay for Base Editors

To refine the understanding of sequence features that govern base editing outcomes, a comprehensive and unbiased approach to characterizing base editors was sought. Libraries of 4,000 or 12,000 oligonucleotides up to 176 nt long encoding unique 20-nt sgRNA spacers were designed and paired with target sequences (35, 56, or 61 bp in length) that contain an NGG or NG protospacer adjacent motif (PAM) to direct Streptococcus pyogenes Cas9 (SpCas9) (Cong et al., 2013; Jinek et al., 2013; Mali et al., 2013) or Cas9-NG, an engineered variant with broadened PAM compatibility (Nishimasu et al., 2018), to the center of each target site (FIG. 1A). Targets included randomly selected wild-type human genomic sequences that flanked partially synthetic base editor target sequences with highly variable sequence compositions, or disease-associated (pathogenic and likely-pathogenic) human genomic sequences selected from the NCBI ClinVar database (July, 2018) and the Human Gene Mutation Database (HGMD, v.2017_4 SNVs) (Landrum et al., 2016; Stenson et al., 2014). Protospacers were cloned upstream of SpCas9 F+E-modified hairpins with improved stability and folding properties (Chen et al., 2013), and a G was added to the 5′ end of spacers that did not natively start with G to ensure efficient transcription from the U6 promoter (Ma et al., 2014). Libraries were cloned into a plasmid that supports Tol2-transposon mediated genomic integration, sgRNA expression, and hygromycin selection for cells with integrated library members (Arbab et al., 2015; Barkal et al., 2016; Shen et al., 2018; Sherwood et al., 2014; Urasaki et al., 2006).

The genomes of mouse embryonic stem cells (mESCs), human HEK293T cells, and human U2OS cells were stably integrated with ≥38,538 unique library cassettes, and transfected with a base editor expression plasmid that supports Tol2-transposon mediated genomic integration and blasticidin selection. To detect rare and diverse editing outcomes with high sensitivity, an average coverage of ≥300× per library cassette was maintained throughout the process. After five days, genomic DNA was collected from treated cells and untreated cells as a control, amplified the library cassettes, and performed high-throughput sequencing (HTS) of the target sites at an average sequencing depth of ≥4,000× per target. This high sequencing depth maximized the number of unique library members that were suitable for downstream analysis despite variability among the representation of library members.

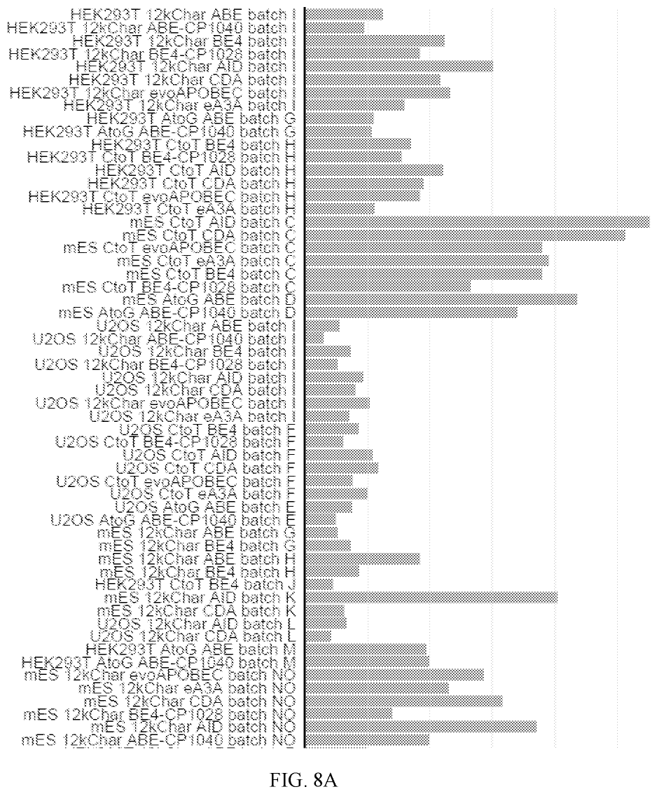

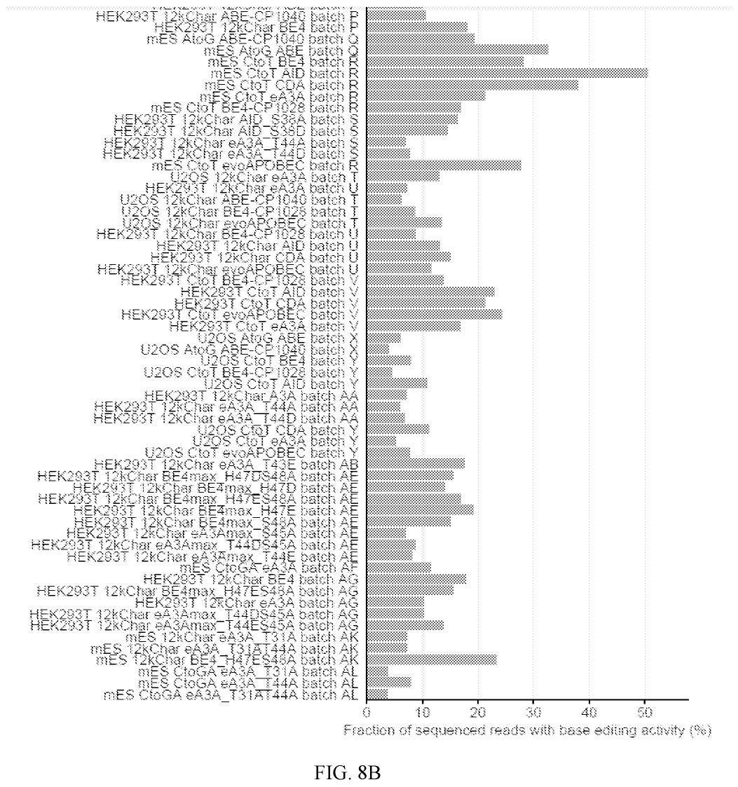

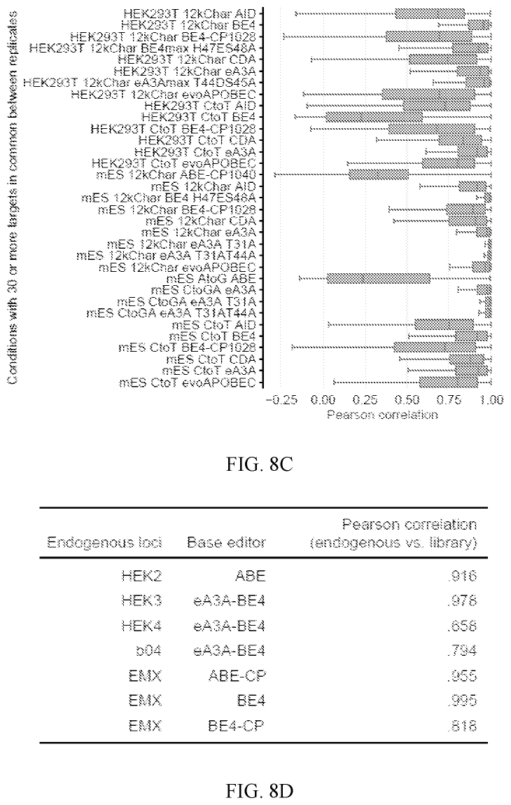

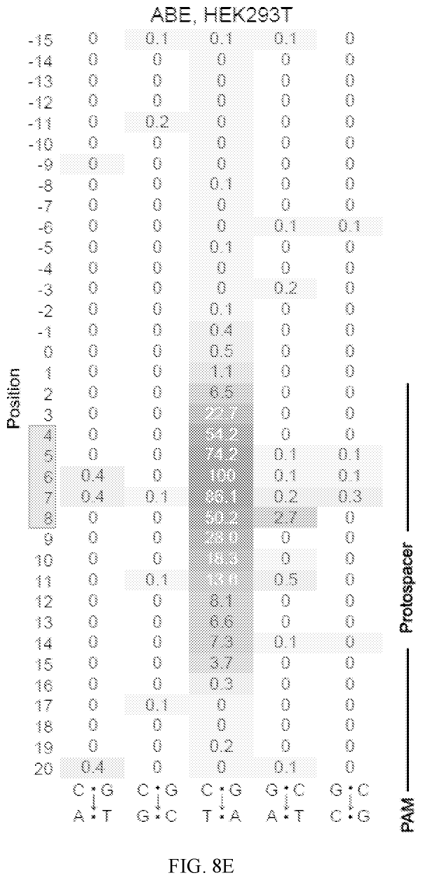

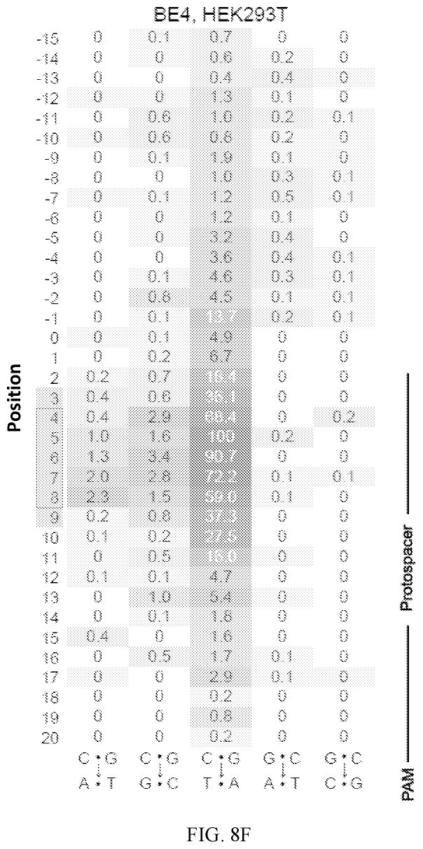

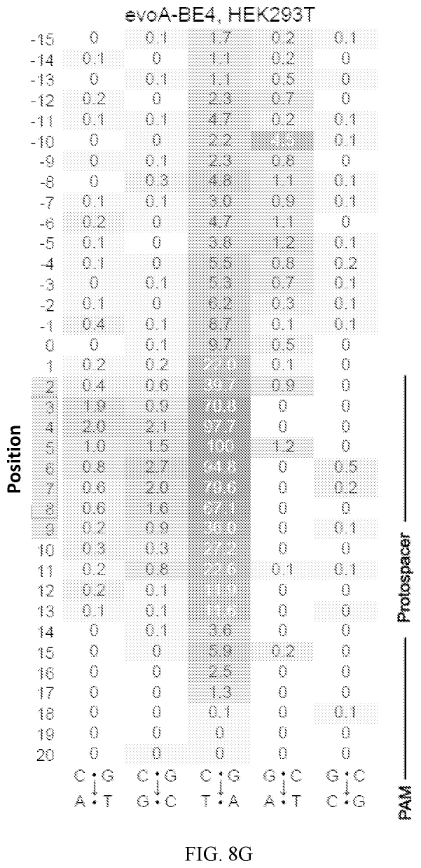

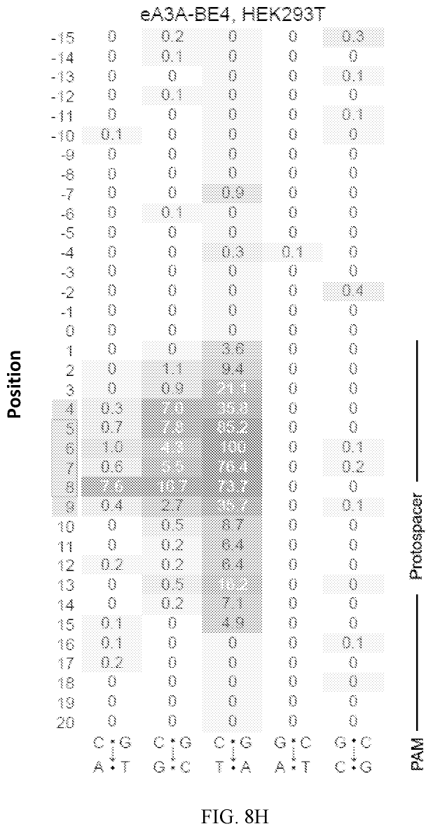

Using this approach, six commonly used CBEs in the NLS- and codon-optimized BE4max architecture were studied (bpNLS-deaminase-Cas9 D10A-2× uracil glycosylase inhibitor (UGI)-bpNLS) (Koblan et al., 2018): BE4max (referred to hereafter as BE4), circularly permuted CP1028-CBEmax (BE4-CP), evoAPOBEC1-BE4max (evoA-BE4), AID (AID-BE4), CDA1-BE4max (CDA-BE4), and engineered APOBEC3A (eA3A-BE4) (Gehrke et al., 2018; Huang et al., 2019; Komor et al., 2017; Thuronyi et al., 2019). Additionally, two ABEs: ABEmax (bpNLS-wt TadA-evolved TadA*-Cas9 D10A-bpNLS, referred to hereafter as ABE) and circularly permuted CP1041-ABEmax (ABE-CP) (Gaudelli et al., 2017; Huang et al., 2019) were studied, for a total of eight base editors spanning a diverse range of editing window sizes and sequence preferences. Two biological replicates per base editor and per cell type were performed, and average editing efficiencies (frequency of target-modified outcomes among total sequenced reads) ranging from 2.9% to 58% (FIGS. 8A-8B) were observed. The resulting data from 2.1 billion sequencing reads was processed, including quality filtering, identification and removal of PCR recombination products, sequence alignment, tabulating editing outcomes, adjusting treated conditions with matched untreated data, and adjusting for batch effects (STAR Methods) to obtain a read count distribution with an average of 1,317 reads per library member per experiment.

Data from library members with low read count were filtered to accurately calculate editing efficiency (fraction of sequenced reads with edited outcomes) and outcome purities (frequency of a given outcome among all edited reads). Between biological replicates, the frequency of base editing outcomes among edited reads at library targets was consistent (median Pearson's R=0.87 across 33 conditions, FIG. 8C) across editors, libraries, and cell types. Editing outcomes at library control sequences taken from the human genome were also consistent with editing outcomes at endogenous loci across five base editors with both narrow and broad editing windows (interquartile range (IQR) of R=0.79-0.98, FIG. 8D). Together, these observations suggest the data are comprehensive, consistent with endogenous editing, and at a scale not previously assayed in base editing.

### Systematic Characterization of Base Editing Activity

Analysis of base editing characteristics at a modest number of endogenous sites (Gaudelli et al., 2017; Gehrke et al., 2018; Huang et al., 2019; Komor et al., 2016; Thuronyi et al., 2019) is constrained by limited variability among the factors that could affect base editing outcomes, including target sequence composition, target sequence context, and locus-dependent differences in DNA-binding proteins and transcriptional state.

To assess sequence-activity relationships of ABEs and CBEs in a more comprehensive manner, base editing outcomes in a genome-integrated library assay with highly diverse sequence compositions were investigated. The library included 8,142 base editor target sequences with all possible 6-mers surrounding a substrate A or C nucleotide at protospacer position 6, and 2,496 sgRNA-target pairs that collectively contain all possible 5-mers included across positions −1 to 13 (counting the position immediately upstream of the protospacer as position 0). This library was designed to enable the detection of deamination events at virtually any sequence context within the reported editing windows of the eight base editors tested, which collectively span protospacer positions 1 to 11 (Gaudelli et al., 2017; Huang et al., 2019; Komor et al., 2016; Thuronyi et al., 2019). The flanking sequences were randomly drawn from the human genome. This collection of 10,638 library members is referred to as the “comprehensive context library”.

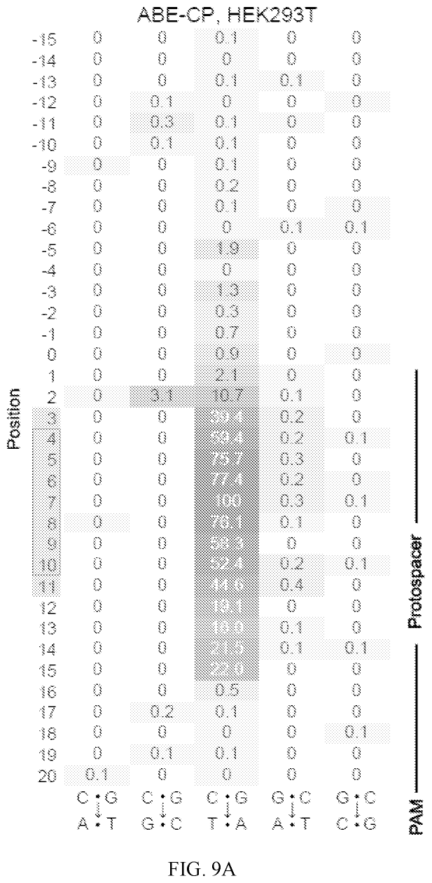

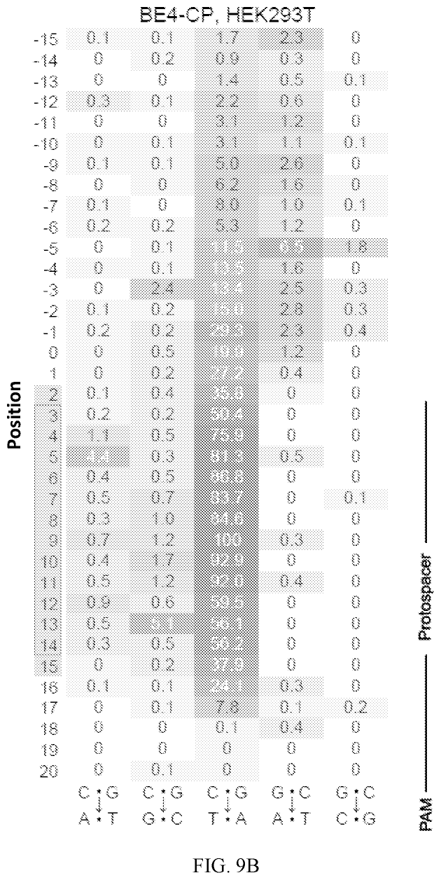

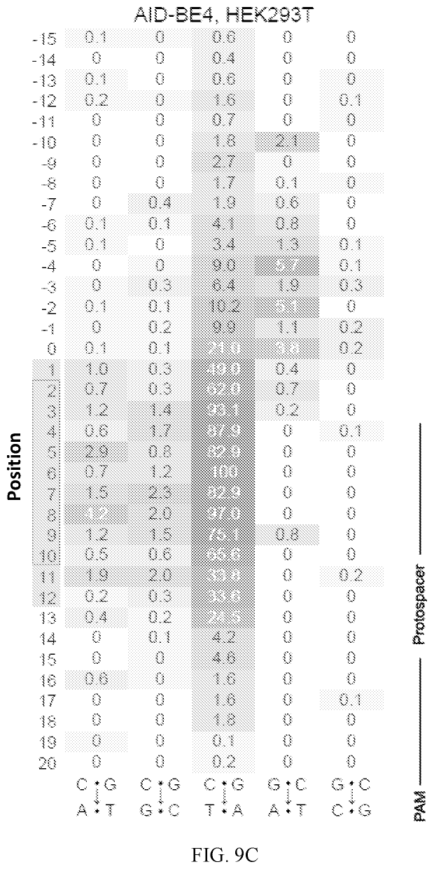

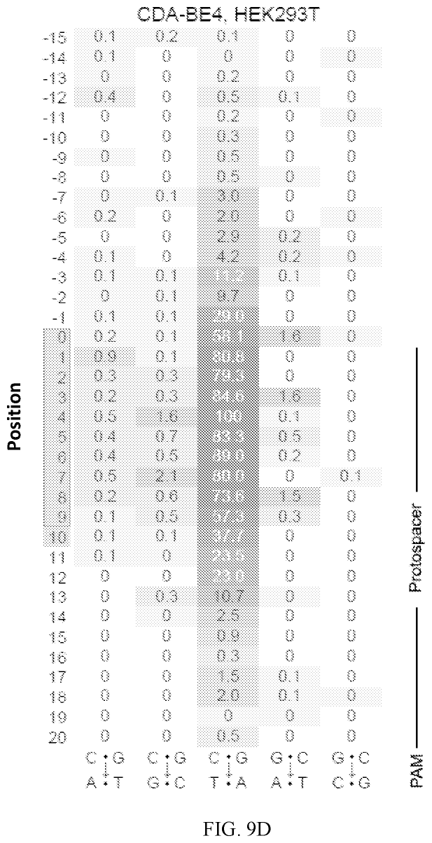

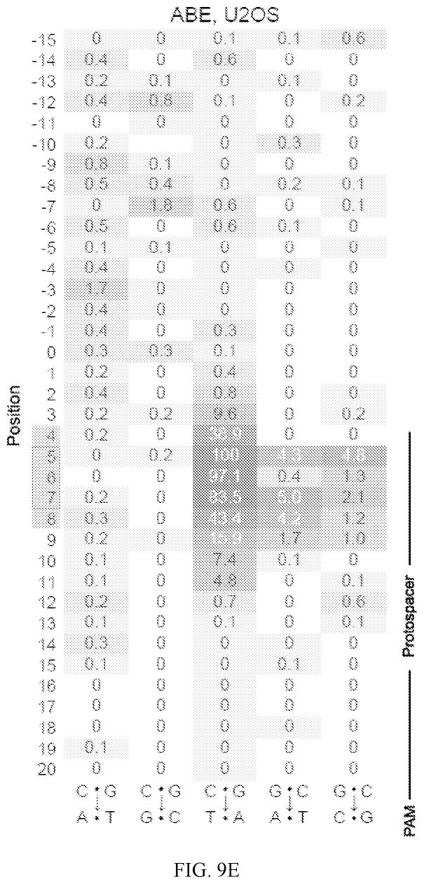

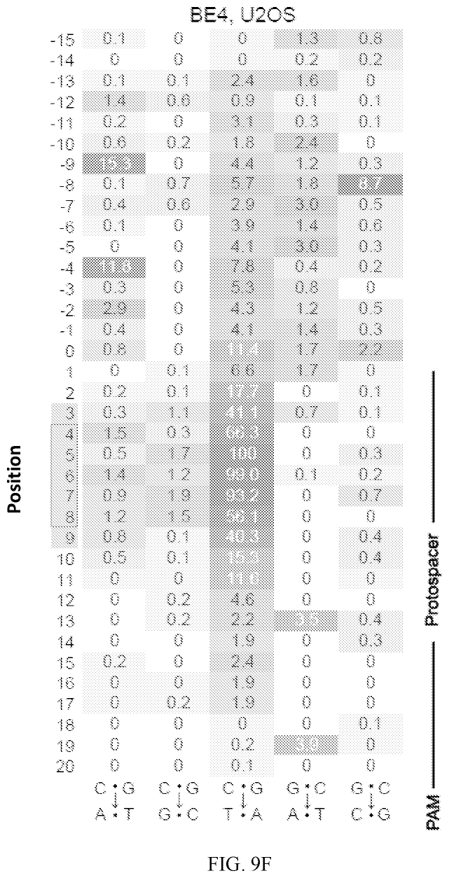

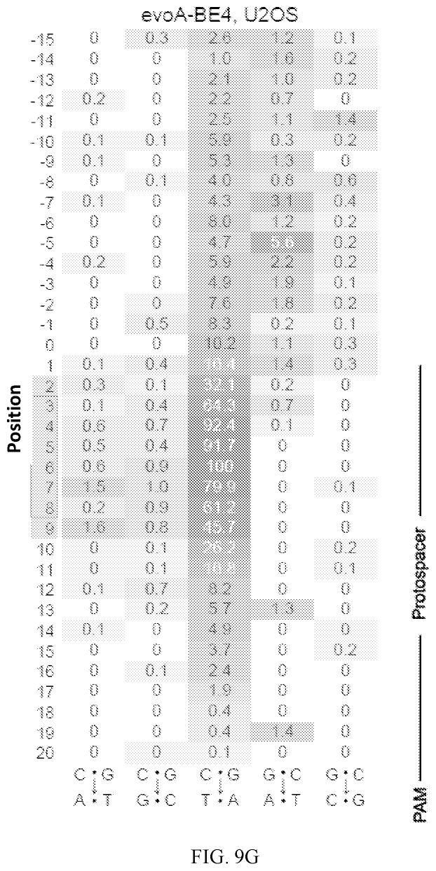

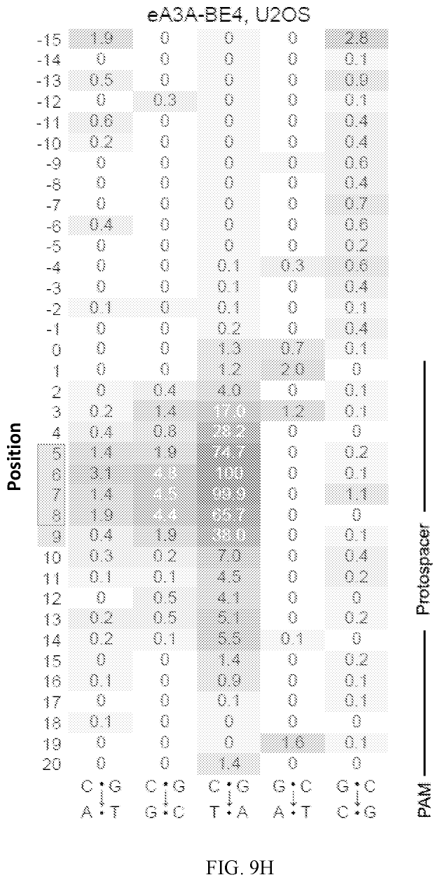

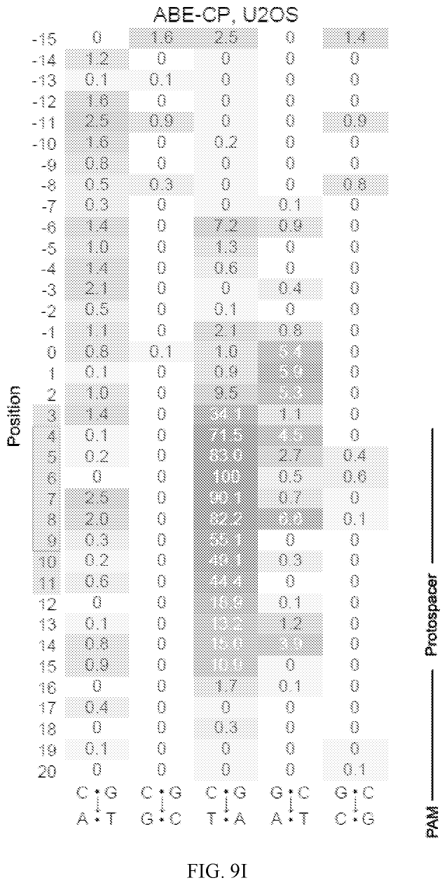

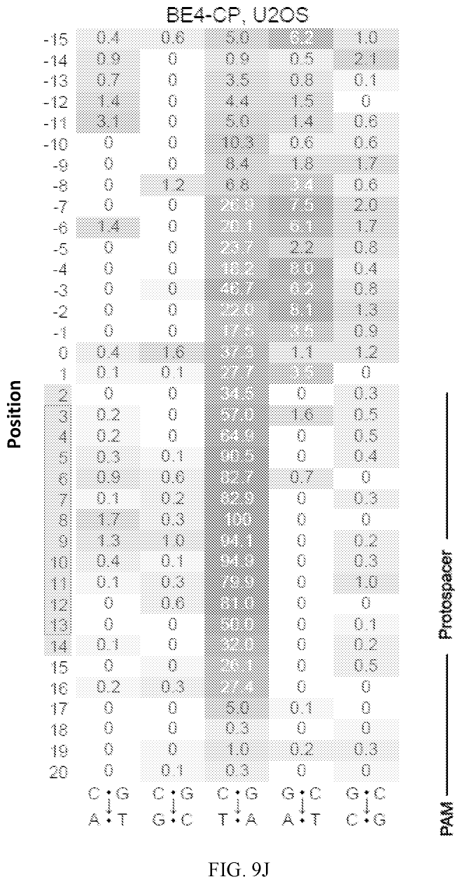

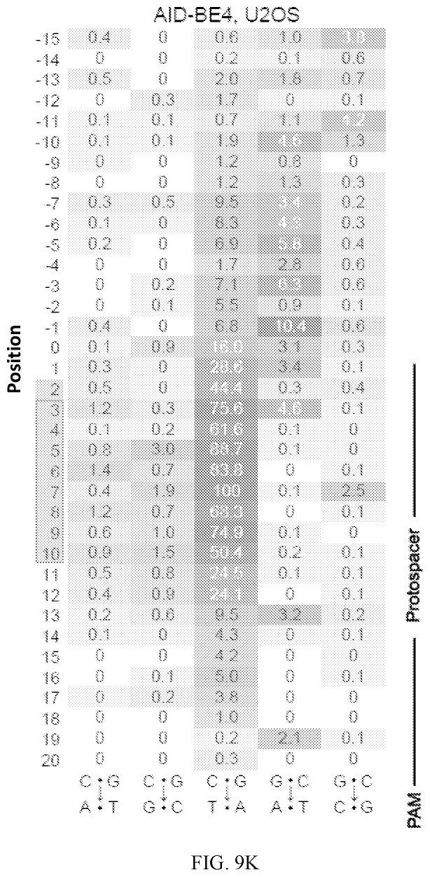

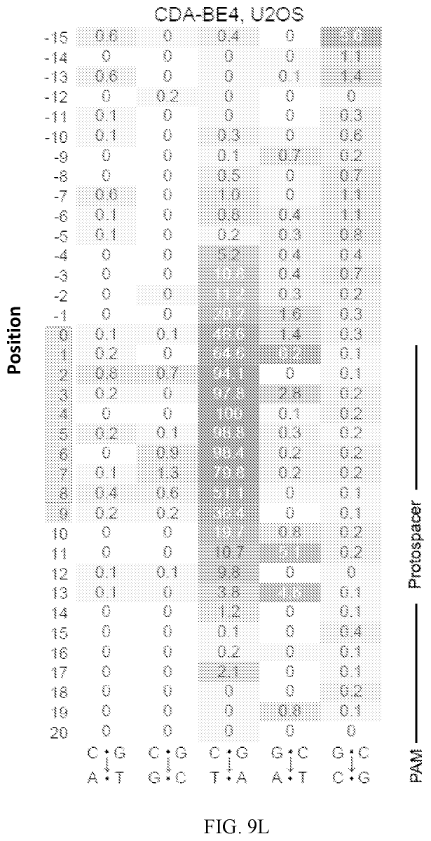

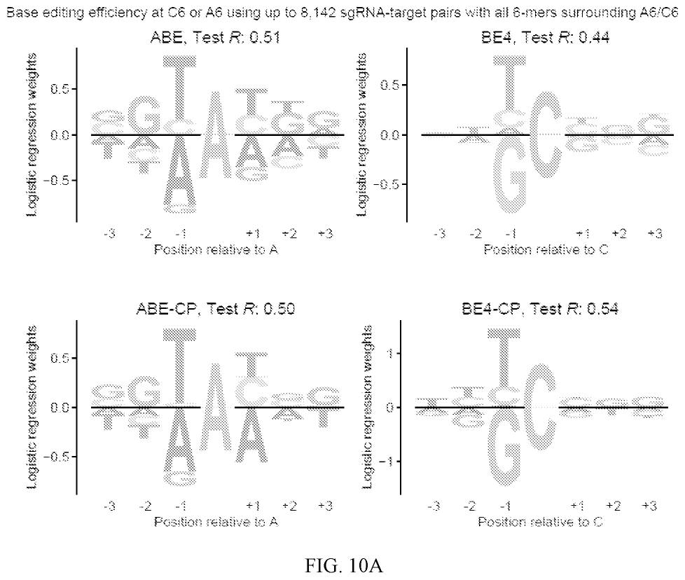

Reads containing indels and base editing outcomes were quantified among the remaining reads from the observed frequencies of all possible nucleotide substitutions from protospacer positions −10 to 35 at individual sequences. Mutations statistically likely to be from DNA sequencing errors were filtered. Robust-rank aggregation was applied to identify editor-specific mutation events that consistently occurred above background frequencies across replicates. These analyses handled all mutation events in an identical manner to minimize bias in the resulting editing profiles (FIG. 1B, FIGS. 8E-8H, FIGS. 9A-9L; STAR Methods).

These profiles revealed variation in editing window positions, distributions of base editing activity, and positional preferences among the eight different base editors tested. BE4 and evoA-BE4 edit at 50% or greater of their maximum frequency at positions 4-8 and 3-8 respectively, consistent with previous reports (Komor et al., 2017; Thuronyi et al., 2019). A unique bimodal editing profile for eA3A-BE4, with an additional peak in activity at protospacer position 13 to up to 18% relative to the maximum editing frequency, was observed that had not previously been reported (Gehrke et al., 2018). The remaining editing windows detected in the assay are in general agreement with, but refine, previous reports (Supplemental Information to Example 1).

As described herein, the editing window is defined using a lowered threshold of >30% maximum editing frequency to include more positions that can undergo substantial base editing. Editors with windows of nine or more nucleotides were classified as wide-window editors, including ABE-CP, BE4-CP, AID-BE4, and CDA-BE4, and eight or fewer nucleotides as narrow-window editors, including ABE, BE4, evoA-BE4, and eA3A-BE4.

### Sequence-Activity Relationships for Common Base Editing Outcomes

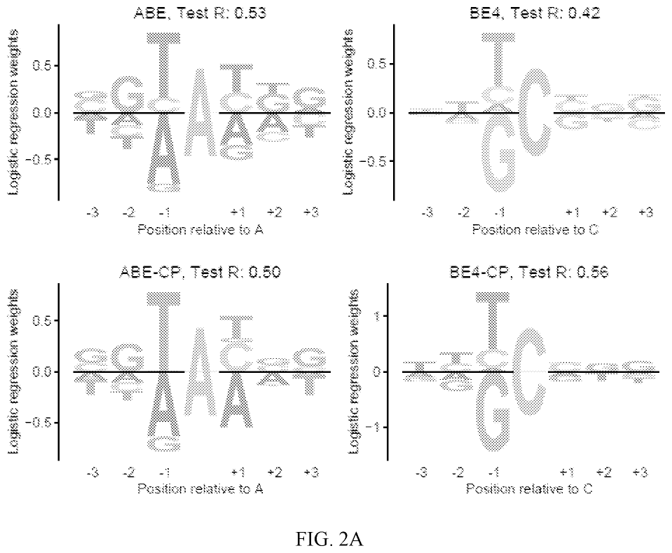

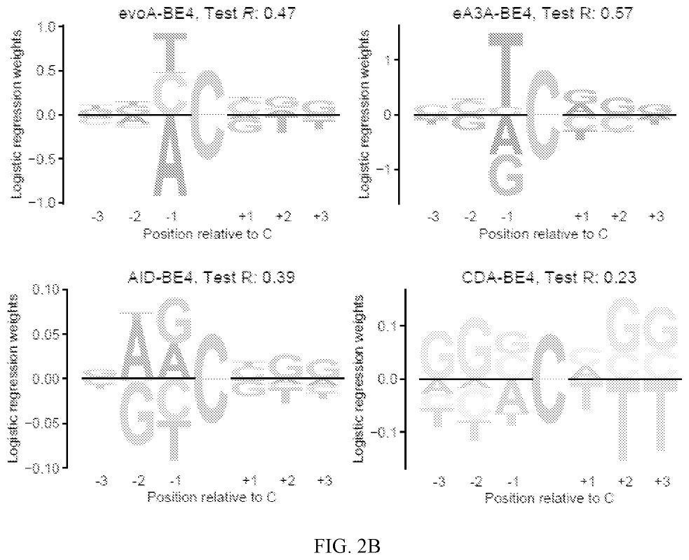

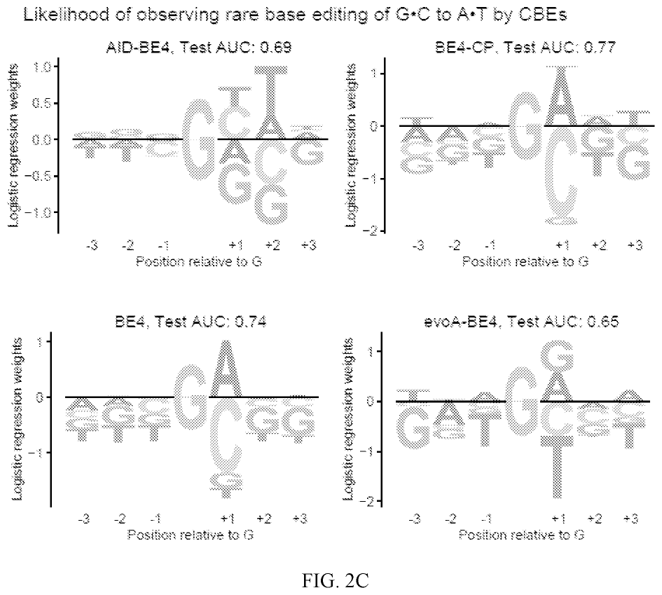

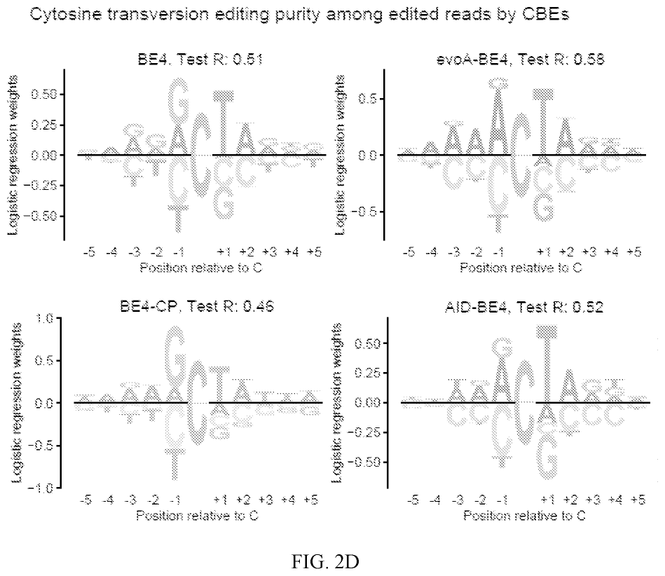

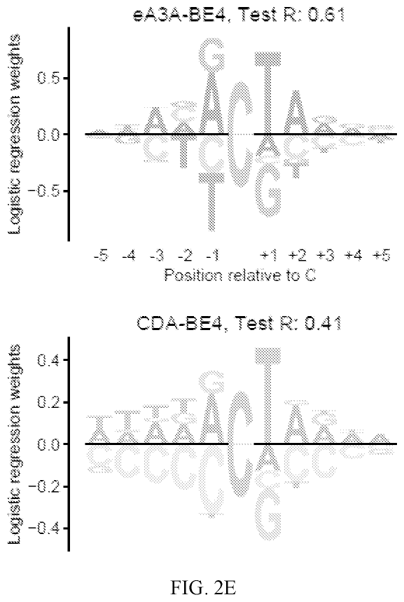

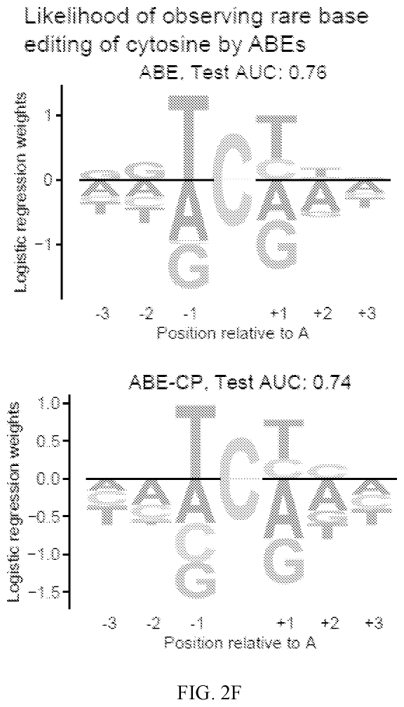

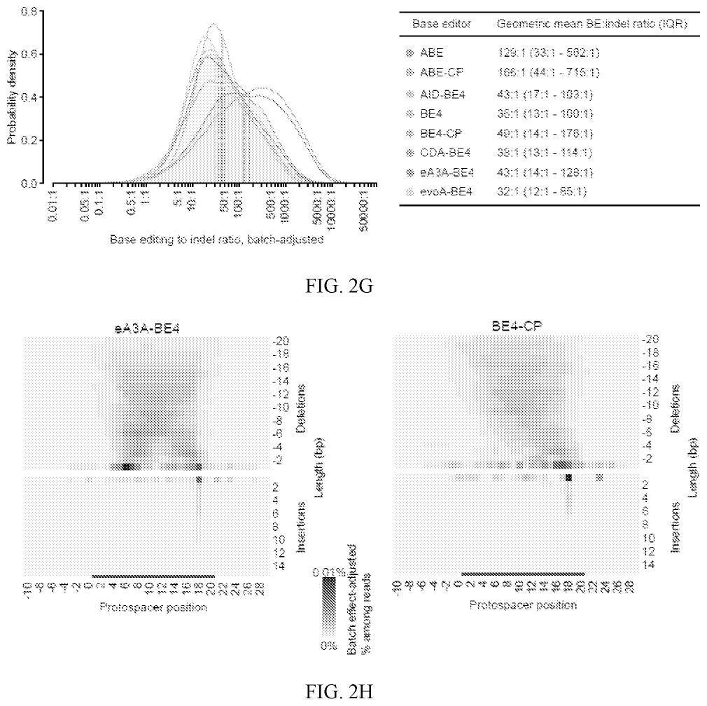

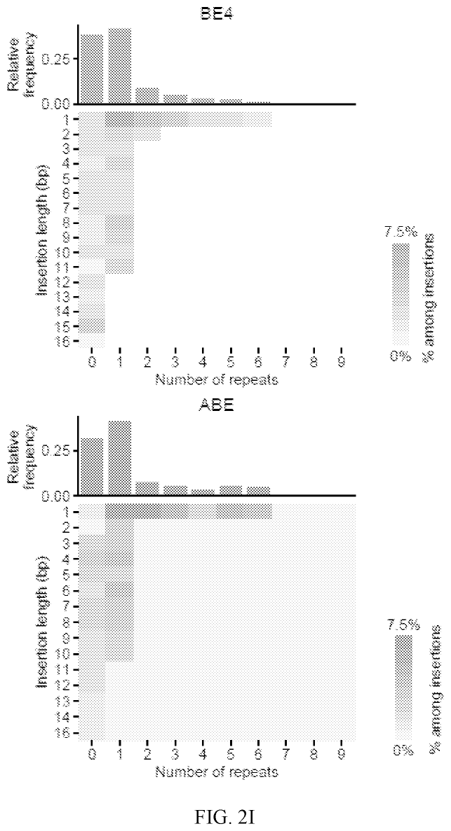

While deaminase-specific sequence preferences have been reported to affect nucleotide conversion efficiencies of some base editors (Beale et al., 2004; Komor et al., 2016; Liu et al., 2018), sequence-activity relationships of base editors have not been characterized in depth. Sequence motifs were generated for various base editing activities, such as editing efficiency, by using logistic regression to predict activity from target sequence context, and depict the learned weights as a sequence logo (FIGS. 2A-2F; Supplementary Information). Motifs described in this manner consider each position independently and are intended for data visualization.

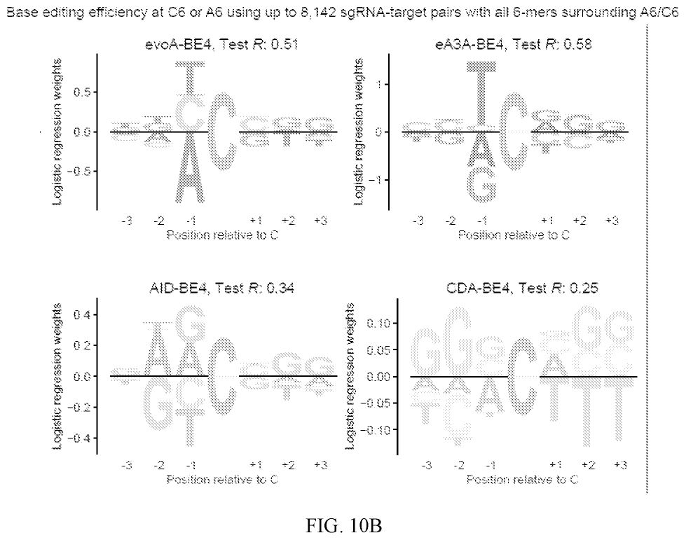

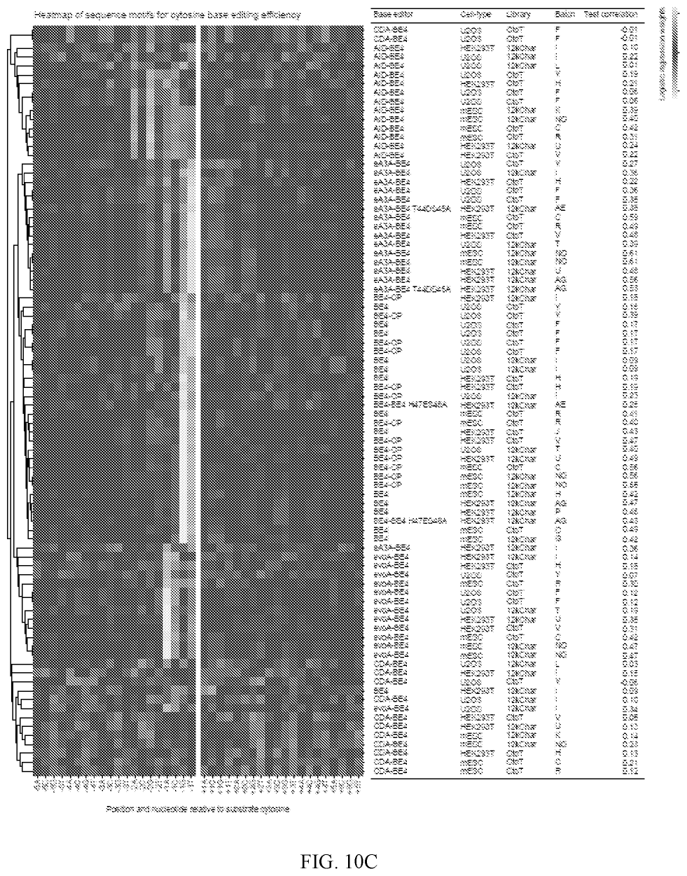

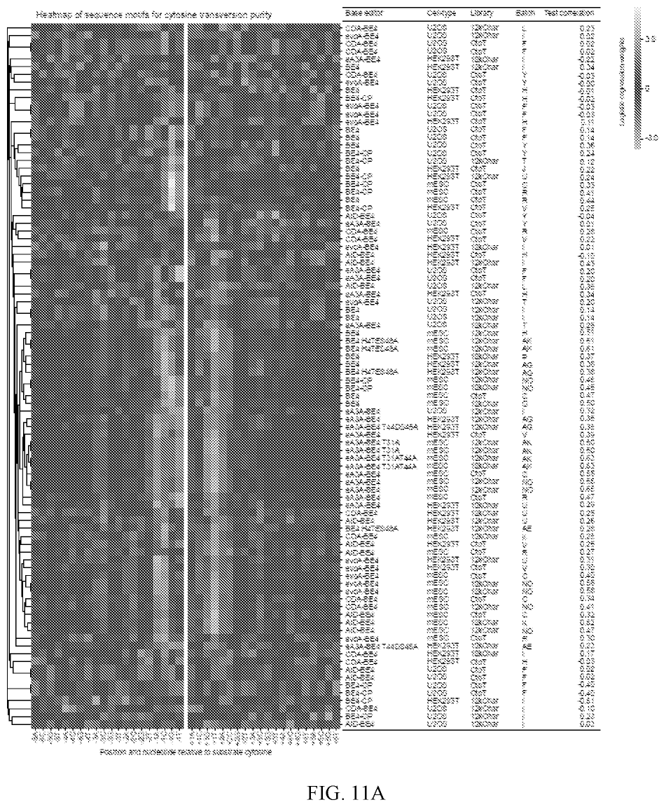

Sequence motifs were first calculated for the efficiency of canonical base editing activity in which CBEs convert C⋅G to T⋅A and ABEs convert A⋅T to G⋅C. Motifs for each editor were obtained at ≥7,091 unique substrate nucleotides in their editing windows at ≥5,292 target sequences (FIG. 2C), that were consistent across cell types and biological replicates (FIG. 10C). These findings identify sequence context as an important determinant of editing activity across all editors with the exception of CDA-BE4, for which only 5.3% of the variance in editing efficiency is explained by target motifs in held-out sequences (variance explained=R²) compared to 15-32% on average across all other base editors.

Interestingly, it was observed that evoA-BE4, which emerged from laboratory evolution to gain activity at GC motifs, acquired a relative aversion to AC targets. This newly acquired anti-preference was previously undetected from analyses at a smaller number of endogenous loci (Thuronyi et al., 2019), likely due to the general increase in base editing activity of evoA-BE4 at all target sequence contexts, including AC, relative to BE4. Similarly, it was found that ABE maintains a preference against AA despite its laboratory evolution that increased activity at sites with adjacent As (Gaudelli et al., 2017). These findings demonstrate that systematic characterization of base editing outcomes at a large number of diverse sequences can reveal CBE and ABE sequence preferences with greater sensitivity than before.

### Non-Canonical Nucleotide Conversions by Base Editors

The analysis revealed several non-canonical editing outcomes. G⋅C-to-A⋅T editing activity by the wide-window editors BE4-CP and AID-BE4 at PAM-distal positions 0 to −5 with mean frequencies of 1.0% and 1.8% among edited reads was observed, respectively (FIG. 1B and FIGS. 9A-9D), in contrast to the narrow-window editors evoA-BE4 and BE4 at 0.32% and 0.43% among edited reads, respectively. These rare outcomes had sequence motifs strongly resembling the reverse complement of each editor's primary cytosine editing activity (for example GA instead of TC, for BE4 and BE4-CP), suggesting that they occur via opposite-strand cytosine deamination (AUC=0.65-0.77, P<5.9×10⁻³, Mann-Whitney U; FIGS. 2D-2E). These G⋅C-to-A⋅T edits are likely inhibited by sgRNA:DNA interactions at protospacer positions 1-20, which may explain their lower overall observed frequency in narrow-window CBEs that do not readily access PAM-distal positions. CDA-BE4 was the notable exception among wide-window editors, which actively edited C⋅G-to-T⋅A at positions −1 to 9 but induced little to no observable G⋅C-to-A⋅T editing.

Cytosine transversion mutations (C to G, or C to A) have previously been observed as a rare CBE outcome (Komor et al., 2016, 2017; Nishida et al., 2016). A strong dependence of transversion edits on local sequence context that was consistent by editor was observed across cell types and biological replicates (FIG. 11A). A preferred motif of RCTA explained 17-37% of the variance among held-out sequences across all CBEs (FIG. 2F). Particularly high transversion frequencies were observed from the narrow-window editor eA3A-BE4 (FIGS. 1B-1I), which averaged 12% transversions relative to the maximum C⋅G-to-T⋅A editing frequency, and a skewed ratio of C-to-G over C-to-A transversion outcomes (˜3:1 for eA3A, compared to ˜3:2 for the remaining CBEs). Together, these results reveal that local sequence context and deaminase choice can influence the frequency and specific outcome of rare CBE transversion editing events.

Rare editing outcomes from ABEs were also identified (FIGS. 1B-1I). The unexpected conversion of C to G, or C to T at protospacer position 6 averaging 0.34% and 0.62% of edited reads for ABE-CP and ABE, respectively, was also observed. These rare outcomes were accurately predicted by the TCY sequence motif, achieving AUC=0.75-0.78 on held-out target sequences (P<6.7×10⁻²³, Mann-Whitney U; FIG. 2G), that strongly resembles the motif for canonical ABE adenine-to-guanine conversion activity (TAY), but is instead centered around. The similarity between these motifs suggests that these rare events occur from direct cytosine deamination by the TadA* active site. Notably, comparable relative frequencies of C⋅G-to-G⋅C and C⋅G-to-T⋅A conversion by ABE cytosine editing were observed, reminiscent of CBE product distribution in early-generation editors that lack UGI (Komor et al., 2016, 2017; Nishida et al., 2016). These observations are consistent with, and extend, a recent report of cytosine editing by ABEs (Kim et al., 2019).

Collectively, these results illuminate sequence- and deaminase determinants of non-canonical ABE and CBE editing outcomes, suggest potential mechanisms of opposite-strand CBE editing, and deepen the understanding of ABE editing of cytosines.

Characterization of Indels Resulting from Base Editors

To date, the factors that determine indel frequency and outcomes in base editing experiments have not been well characterized. Consistent with prior reports, generally high ratios of desired base edits to undesired indels were observed, averaging 40:1 for the six CBEs and 145:1 for the ABEs (geometric means), although BE:indel ratios varied substantially by target; for example, IQRs for CBEs were 15:1 to 100:1 (FIG. 2G; Supplementary Information). Wide-window editors generally induced indels at lower relative frequencies than narrow-window editors.

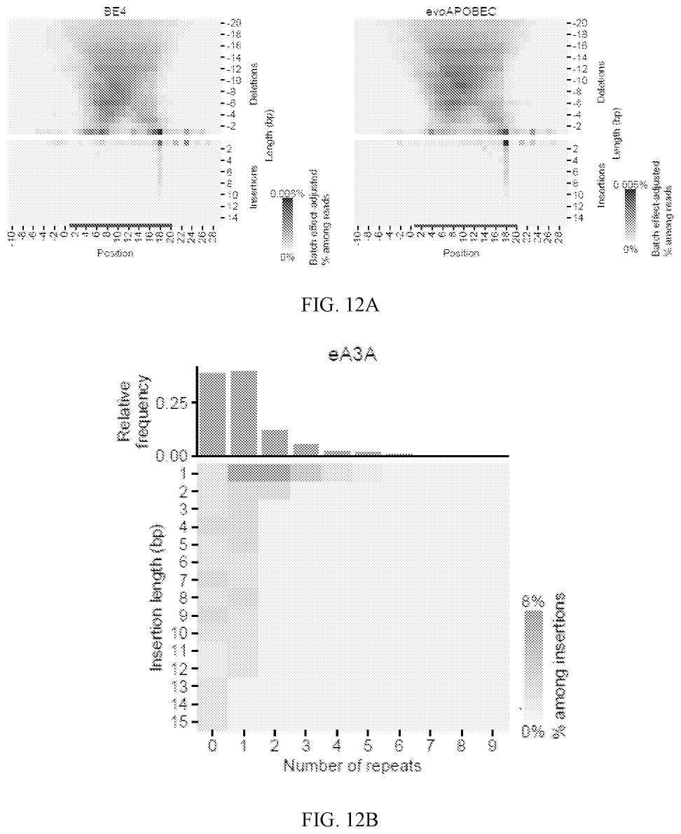

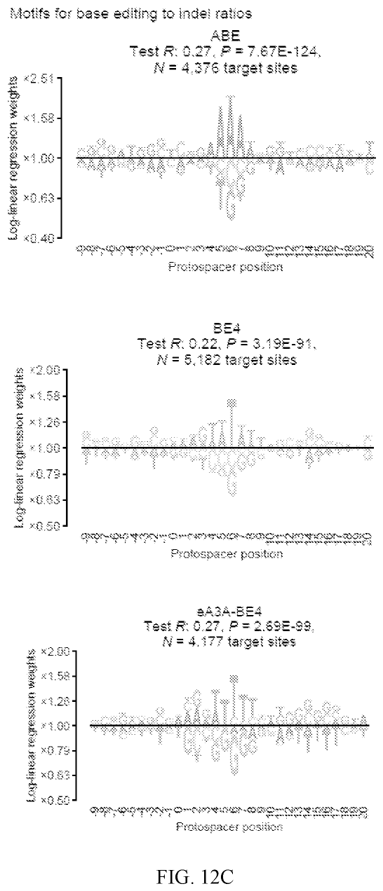

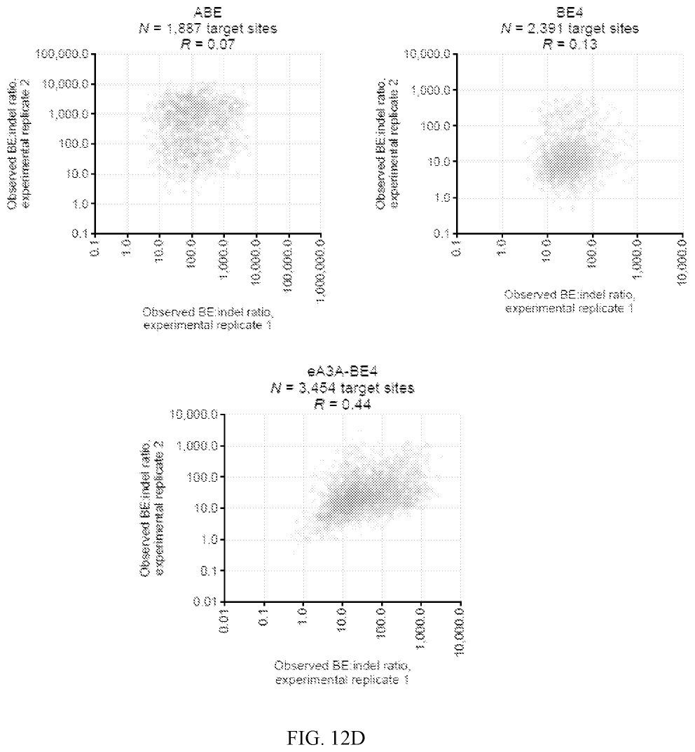

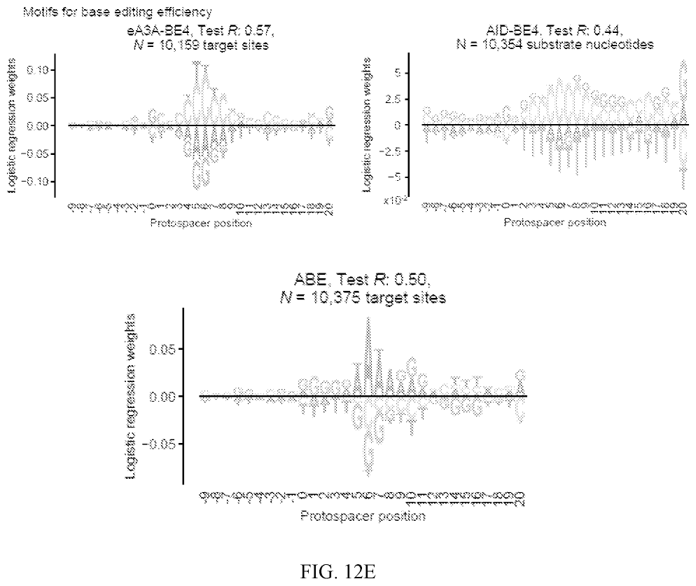

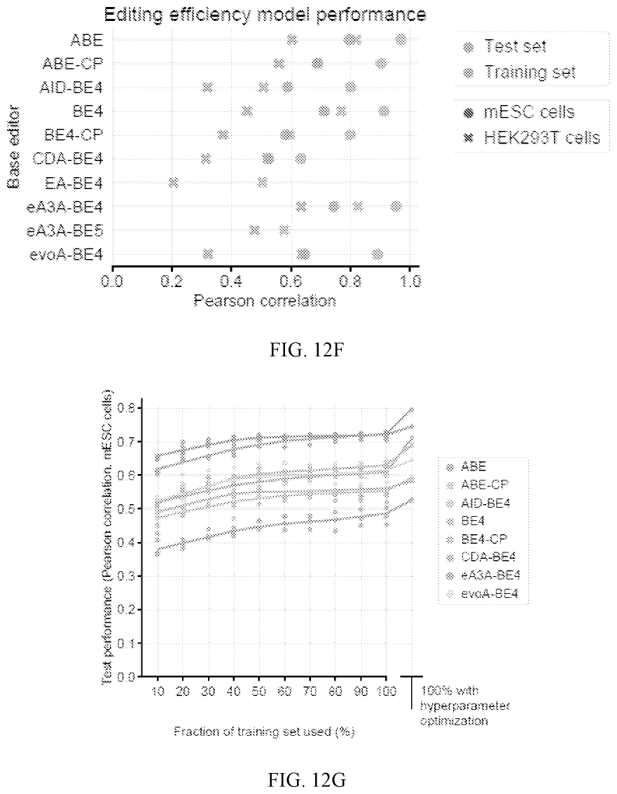

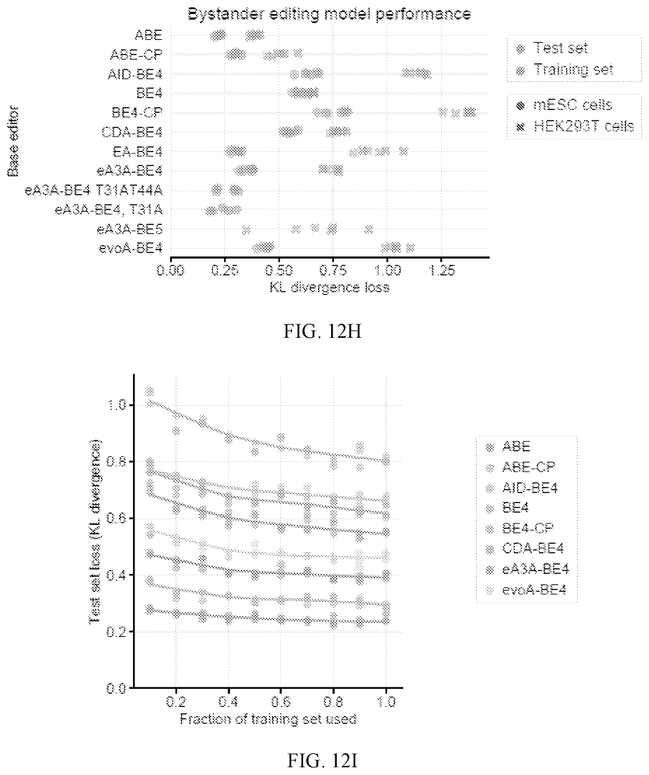

The outcome analyses revealed a characteristic positional profile of insertions and deletions specific to base editors (Supplementary Information). Deletions were centered around either the PAM-proximal HNH domain's nick location preceding protospacer position 18, or the PAM-distal deamination peak position for the CBE (often position 6), or spanned these two sites resulting in a peak in outcome frequency at ˜12 bp deletions (FIG. 2H and FIG. 12A), while insertions predominantly consist of single or multiple nucleotide duplications preceding position 18, at the location of the HNH-nick (FIG. 2I and FIG. 12B). The rare insertion outcomes from base editing are similar to, yet distinct from, insertion products of Cas9 nuclease-mediated editing, which are heavily dominated by 1-bp duplications of the nucleotide immediately 5′ of the double-strand break (Shen et al., 2018).

Indel frequencies are largely unaffected by cell type and sequence context. A 1.2-fold increase in BE:indel ratio in HEK293T cells, and 2.1-fold increase in U20S cells relative to mESCs, was observed although neither was statistically significant (FIG. 11D). Strong sequence determinants of indels resulting from base editing were not observed. Sequence motifs trained to predict BE:indel ratios only explain 0.5-8.4% of variation in held-out sequences (P<7.0×10⁻³¹; FIGS. 12C-12D).

Collectively, these analyses provide the first comprehensive characterization of indels that result from base editing. The relative rarity of indels resulting from base editing was confirmed, a minor dependence on cell type and target sequence was observed, and a unique location profile of indel outcomes was determined that was distinct from that of Cas9 nuclease-mediated indels.

### Editing Efficiency Model

Base editing efficiency at endogenous genomic loci depends on a number of factors. Local sequence context determines deaminase sequence-dependent activity, and PAM compatibility affects the accessibility of the target nucleotide to the deaminase. In addition, cell type specific factors, including replication rate, the abundance of repair proteins, and DNA states such as chromatin accessibility and transcriptional activity may affect sgRNA binding and repair of deaminated nucleotides. Since sequence composition is not cell type-dependent, revealing how sequence features affect base editing efficiency has the potential to benefit experimental design across all cell types. While previous reports have assessed how local sequence context at a given target site impacts deaminase efficiency (Gaudelli et al., 2017; Gehrke et al., 2018; Komor et al., 2016), empirical optimization of editor and sgRNA choice is still often necessary due to the lack of simple relationships between target sequence context and base editing outcomes (Huang et al., 2019; Tan et al., 2019; Thuronyi et al., 2019; Villiger et al., 2018).

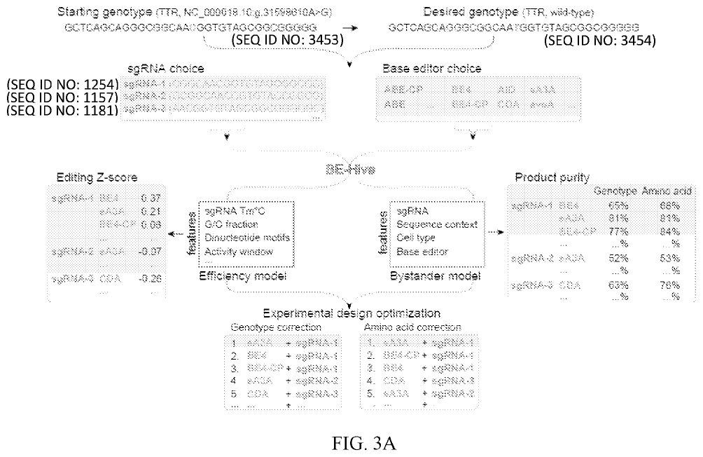

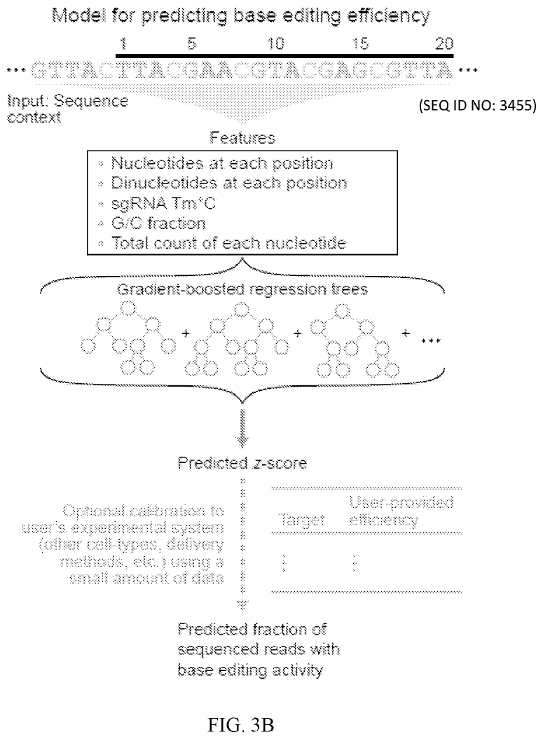

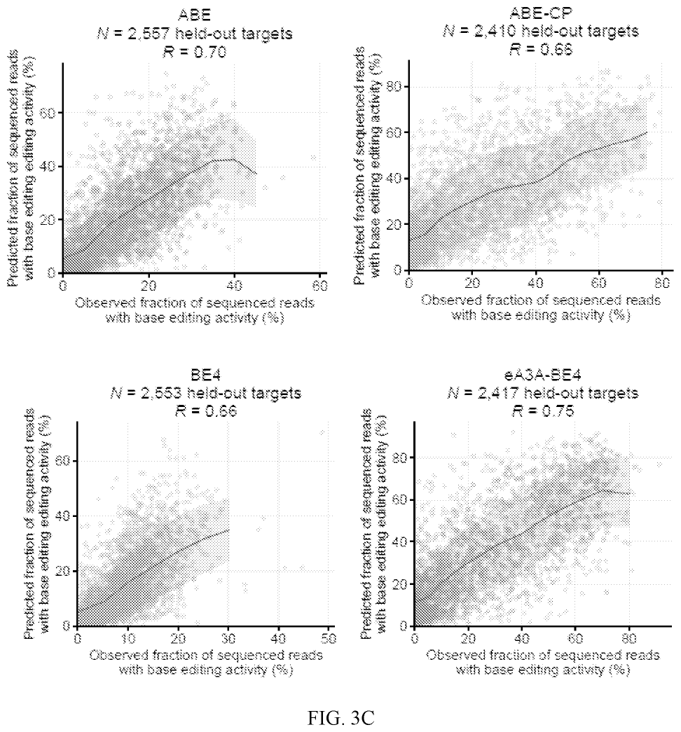

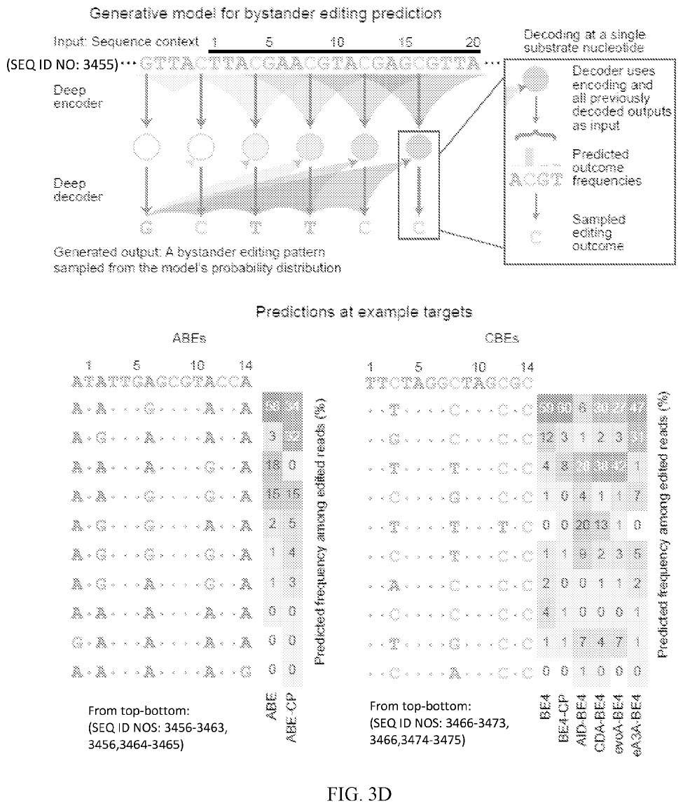

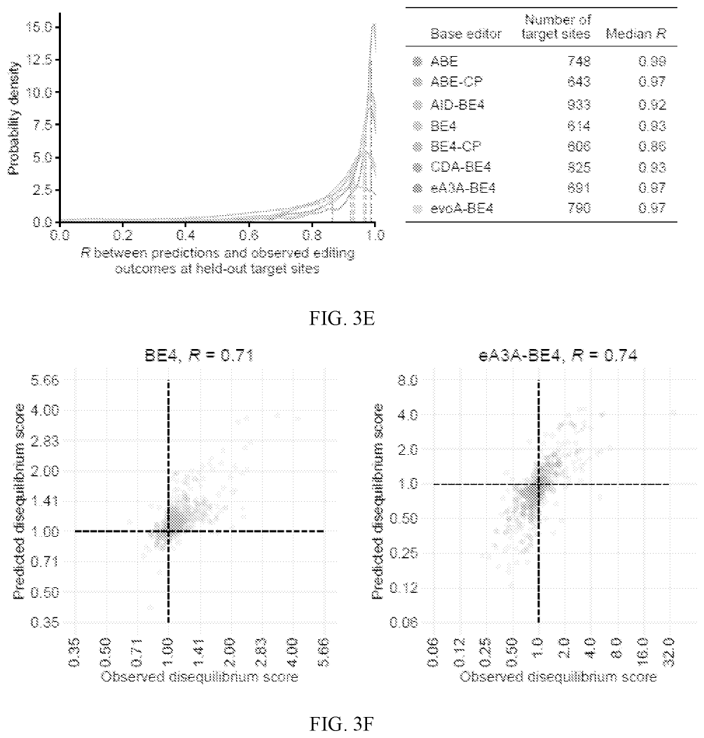

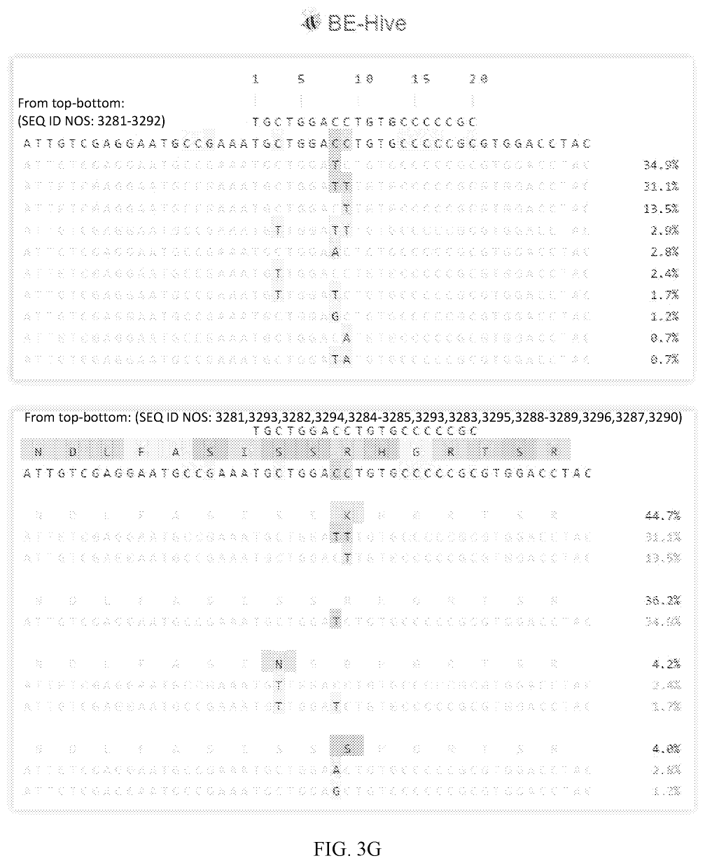

A model to inform the design of base editing experiments including all possible choices of base editors, sgRNAs, and targets to enable a desired phenotype were sought (FIG. 3A; Supplementary Information). The relationship between sequence and base editing efficiency was investigated using the comprehensive context library across two biological replicates in each of three cell types. Sequence motif models were trained and it was found that the learned motifs (FIG. 12E) resemble a combination of each editor's single-nucleotide sequence motif and activity window. To consider higher-order interactions and additional features, gradient-boosted regression trees were applied (FIG. 3B) (Friedman, 2001). These models improved on the performance of logistic regression motifs (R=0.50-0.57) and achieved R=0.69-0.80 for ABEs and R=0.53-0.74 for CBEs in held-out sequences (FIG. 3C and FIGS. 12F-12G) in mES cells. In HEK293T cells, the models achieved R up to 0.60 for ABEs and eA3A-BE4. The tree models found features including sgRNA melting temperature, G/C fraction, and dinucleotide motifs (such as TC for some CBEs and AA for ABEs) were useful in predicting base editing efficiency).

These efficiency models, as with most machine learning models, provide output on an abstract scale by default, however, with a minimal amount of user input, the output can be calibrated to their custom experimental conditions to provide outputs on a more natural scale as the fraction of sequenced reads with any base editing activity. This model design, in contrast with other models developed for CRISPR-related editing efficiency, alleviates the requirement for users to perform additional heuristic interpretation of machine learning model outputs (Doench et al., 2016).

### Bystander Editing Model

“Bystander editing” of non-target C or A nucleotides located near the target nucleotide represents a significant challenge for precision base editing, as ˜70% (1-0.754) of targets have two or more C or A nucleotides within a five-nucleotide window. In many base editing applications, bystander edits that result in silent coding mutations may be innocuous, thus broadening the potential number of desirable editing outcomes. Thus far, design guidelines for avoiding bystander edits have relied on heuristics derived from data at modest numbers (typically 10-100) of sites, and often do not dissect what combinations of target and bystander editing events are most likely to occur, and which targets are amenable to precise single-nucleotide editing or coding correction.

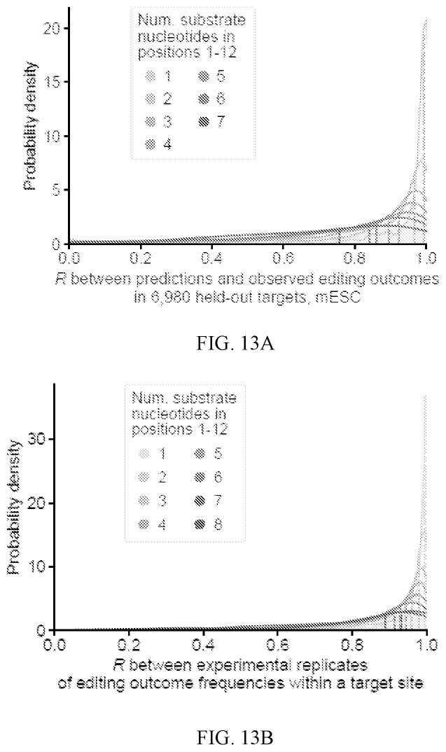

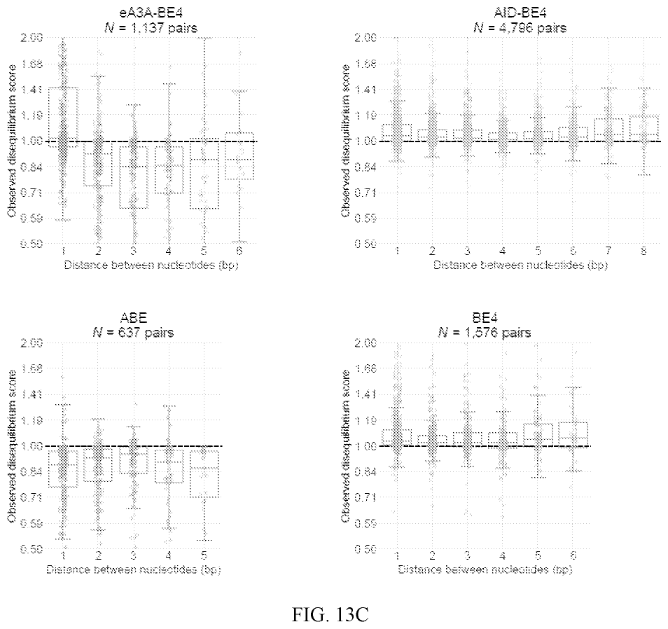

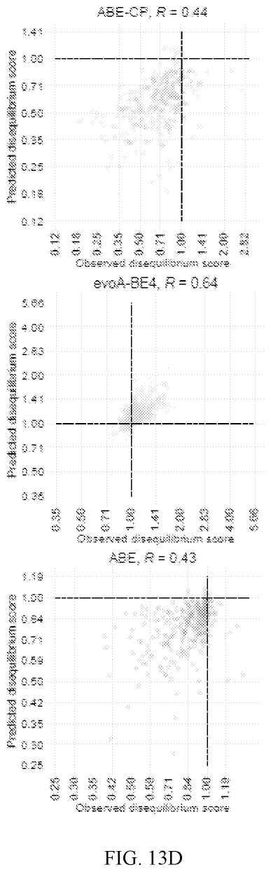

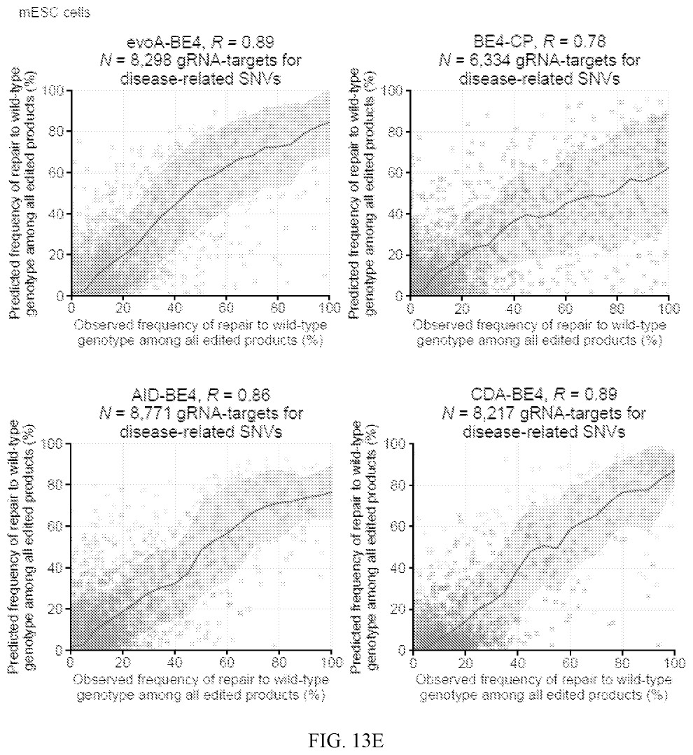

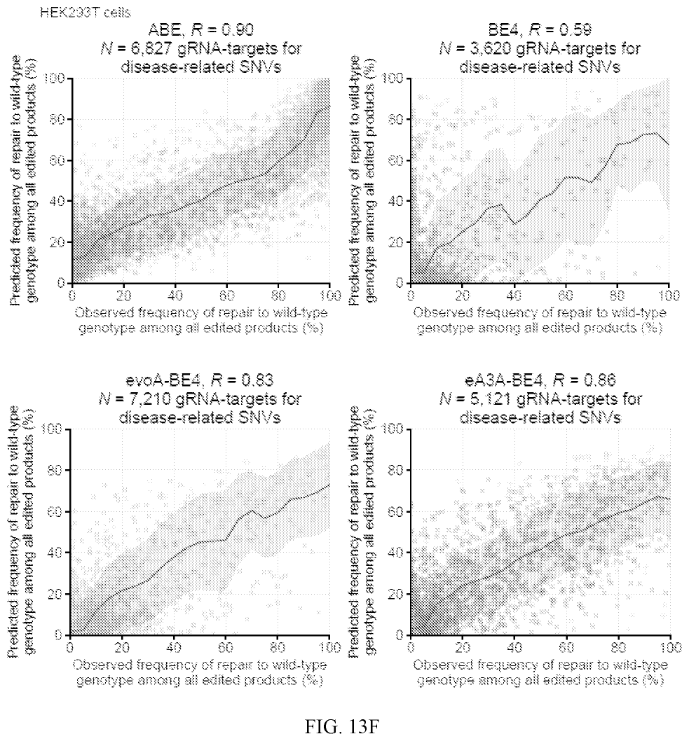

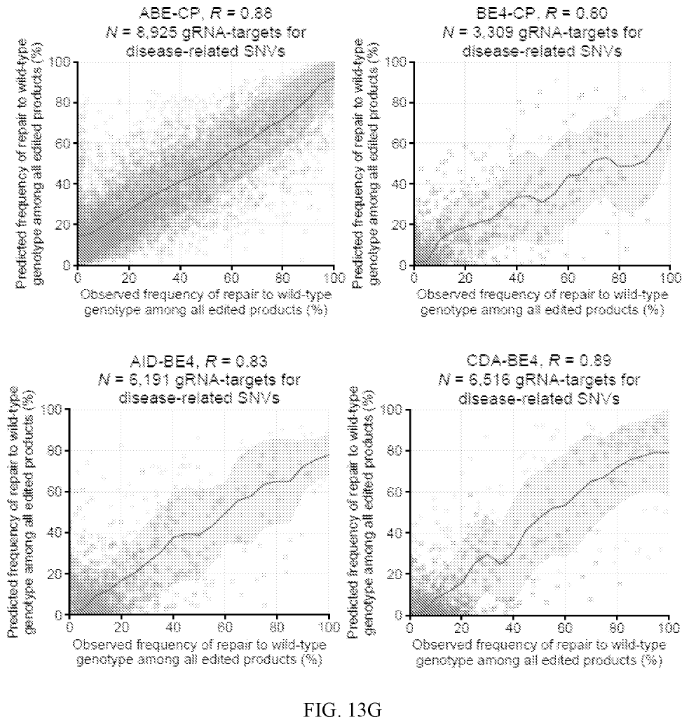

To predict bystander base editing patterns, a deep conditional autoregressive machine learning model (Van Den Oord et al., 2016) was designed that uses an input target sequence surrounding a protospacer and PAM to output a frequency distribution on combinations of base editing outcomes (FIG. 3D; Supplementary Information). Data was randomly split from up to 10,638 sgRNA-target pairs in the comprehensive context library by an 8:1:1 ratio into training, validation, and test data sets to train and test the model. The model predicts all nucleotide substitutions from protospacer positions −10 to 20. To flexibly model editing profiles with any shape, a learned positional bias towards producing an unedited outcome was introduced. An architecture search, ablation analysis, and comparisons to baseline methods (STAR methods) were performed and it was concluded that the autoregressive design and use of a high-capacity decoder were important for predictive performance. Across the six CBEs and two ABEs tested here, the bystander model performed strongly at predicting the frequencies of bystander editing patterns, achieving a median R=0.86-0.99 on ≥606 held-out target sequences in mES cells (FIG. 3E and FIGS. 12H-12I). The model retained strong performance even at target sites with many substrate nucleotides (FIGS. 13A-13B).

Deaminase enzymes and base editors are reported as having varying degrees of processivity, the ability to sequentially catalyze multiple base conversions without releasing the target DNA (Gaudelli et al., 2017; Komor et al., 2016; Love et al., 2012; Nishida et al., 2016; Pham et al., 2003). Base editing processivity can be evaluated using disequilibrium scores, the ratio between observed frequency of two proximal nucleotides both being edited and the expected frequency assuming statistical independence. A large variation in disequilibrium scores of base editors (FIG. 3F and FIGS. 13C-13D; Supplementary Information) that was accurately predicted by the bystander model (R=0.71 and 0.74 for BE4 and eA3A-BE4) was observed, demonstrating that it has learned higher-order conditional editing probabilities.

The editing efficiency and bystander editing models were collectively named “BE-Hive”, freely accessible at crisprbehive.design. Using target sequence as input alone, BE-Hive estimates base editing efficiency and outcomes at the single-nucleotide and coding-level. BE-Hive represents the first tool for designing base editing experiments that comprehensively considers on-target editing efficiency, deaminase and sequence-related preferences for various editing outcomes, and the likelihood of bystander edits to distinguish targets that are amenable to high-precision single-nucleotide editing and coding-sequence correction using a variety of established base editors (FIG. 3G).

### Model-Guided Precise Correction of Pathogenic Alleles

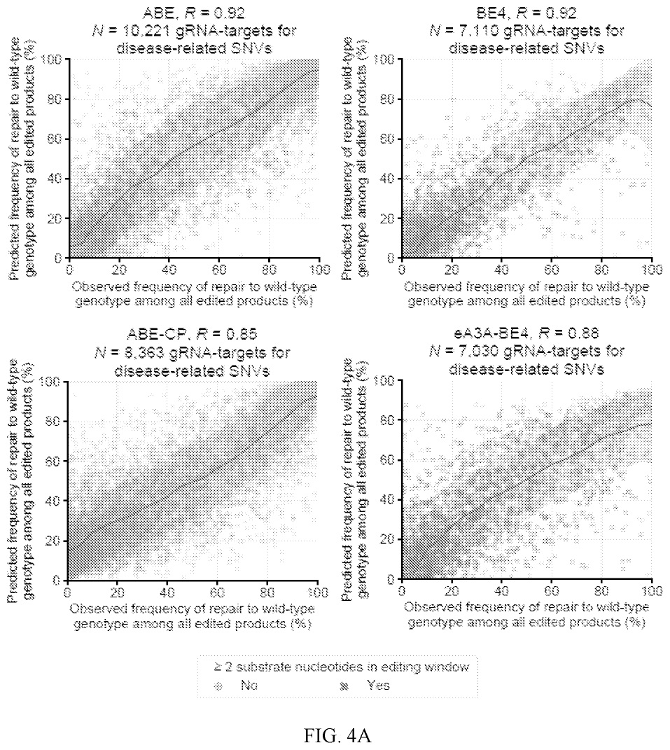

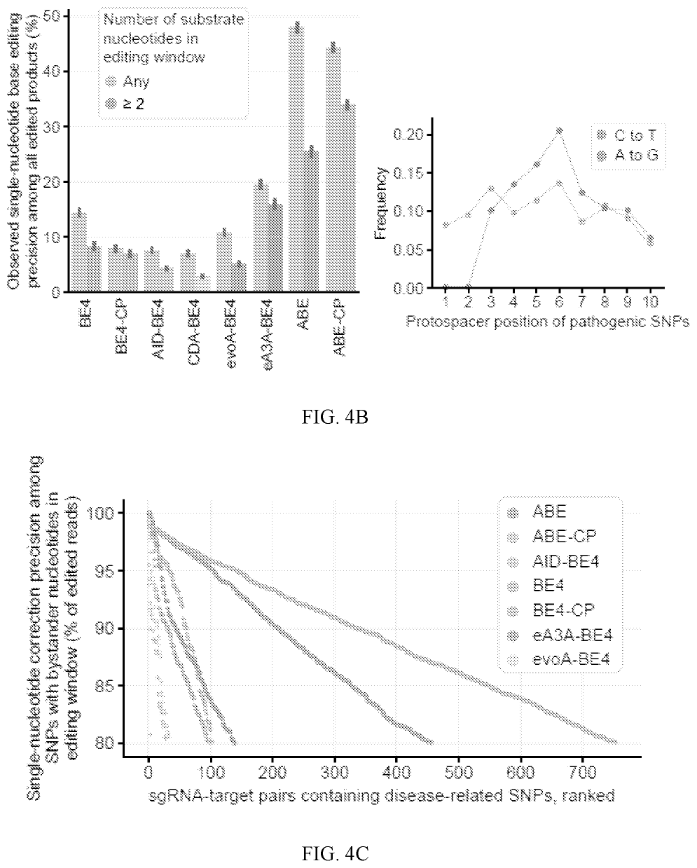

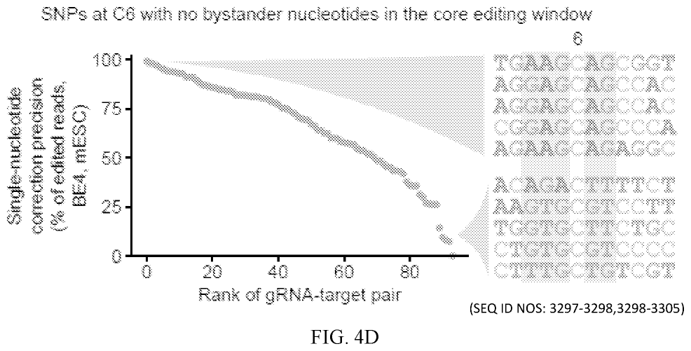

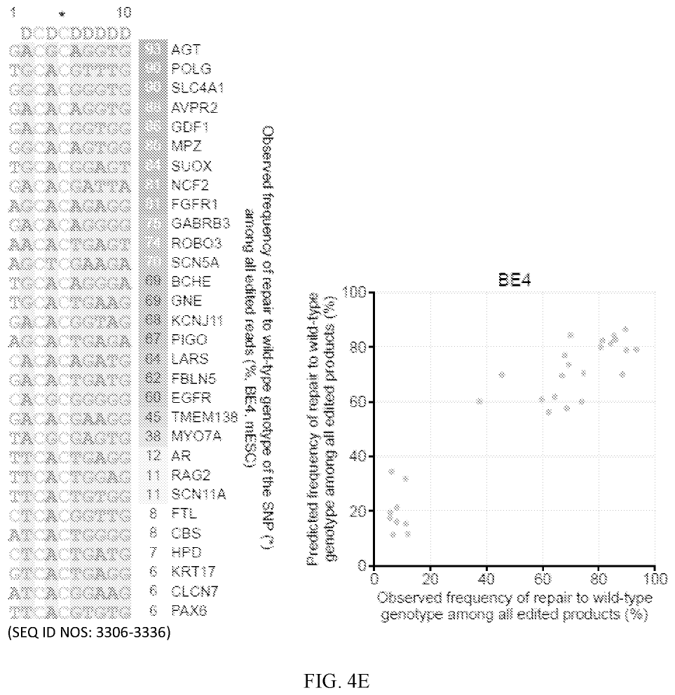

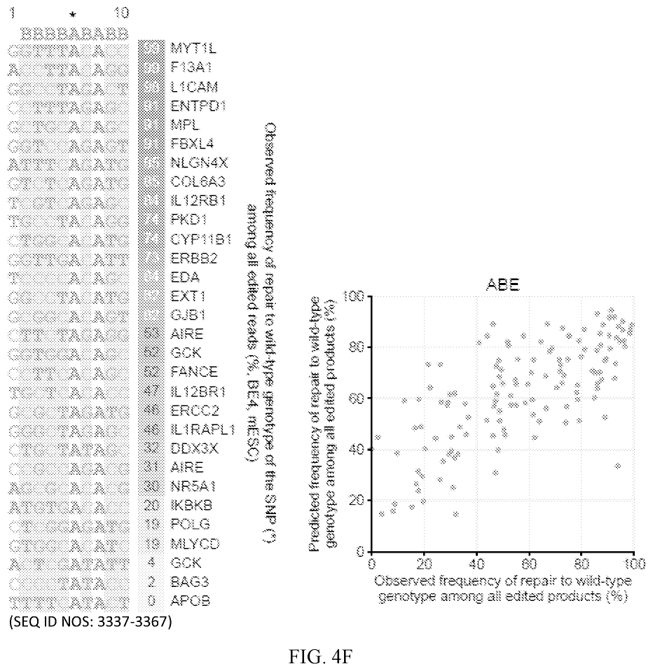

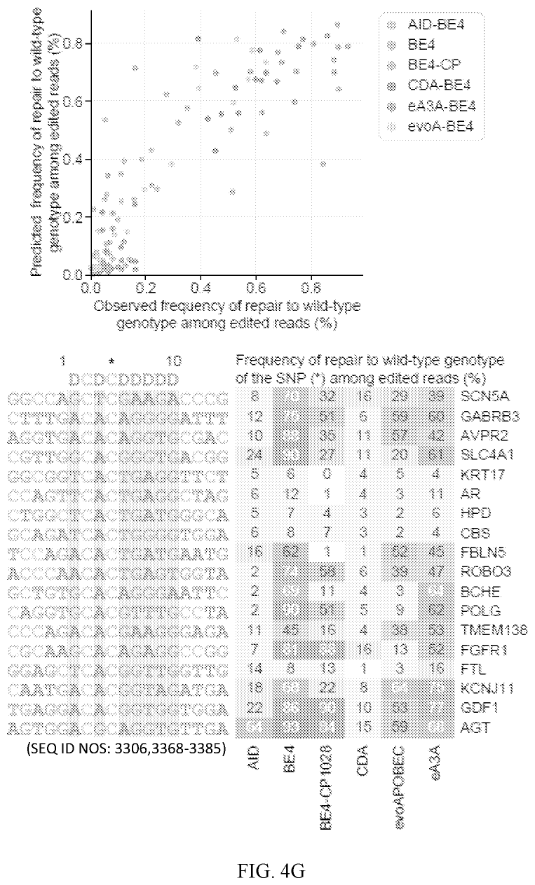

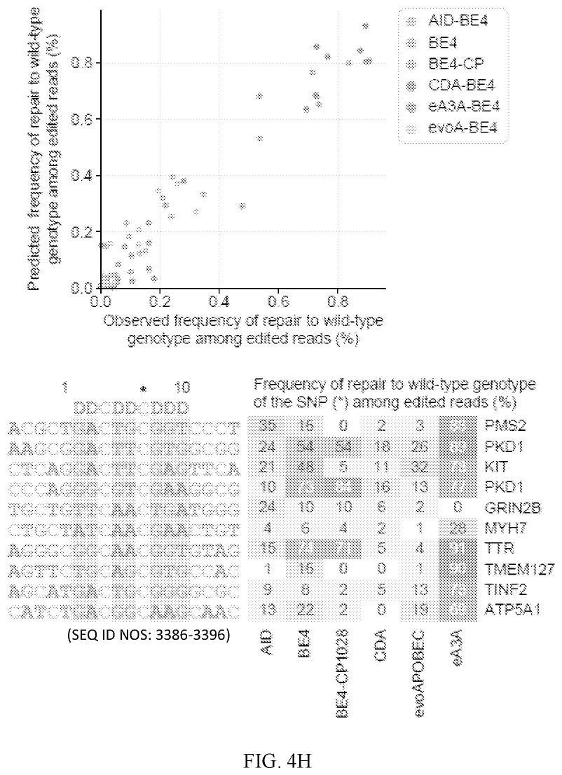

A deeper understanding of base editor sequence-activity relationships would facilitate the selection of optimal base editor and sgRNA combinations that maximize editing efficiency and precise editing of only the intended target nucleotide(s) at a given locus. The ability of the bystander editing model to predict correction of disease-relevant alleles by base editing was examined. A library of 12,000 sgRNA-target pairs for 7,444 unique disease-associated variants from ClinVar and HGMD that are correctable by precise C⋅G-to-T⋅A conversion was designed, which was referred to as the “CBE precision editing SNV library”. Analogously, the “ABE precision editing SNV library” was designed, which assesses precise A⋅T-to-G⋅C editing of ABEs with 12,000 sgRNA-target pairs for 11,585 unique SNV variants. For both libraries of disease-associated SNVs, ˜80% were previously annotated as pathogenic while the remaining were classified as likely pathogenic (Landrum et al., 2016; Stenson et al., 2014). To comprehensively assess the model's performance, the library was intentionally designed to include SNVs in suboptimal protospacer positions and with both high and low correction precision and efficiency as predicted by a preliminary version of BE-Hive. HTS data was obtained for these 24,000 sgRNA-target pairs using the genome-integrated library assay in mouse and human cells for eight base editors. BE-Hive accurately predicted correction precision, which is the fraction of edited reads that contain an exact single-nucleotide edit that corrects the SNV to the wild-type allele, achieving median R=0.89 for ABEs and 0.86 for CBEs in mES and HEK293T cells (FIG. 4A and FIGS. 13E-13G). A ≥90% precise single-nucleotide correction to the wild-type allele at 3,036 SNVs by ABEs and 364 by CBEs was observed.

Precise single-nucleotide correction is less frequent when multiple substrate nucleotides are present in the window, ranging from 2.9% to 16% on average for CBEs and 26% to 34% for ABEs (FIG. 4B). However, 675 unique disease-associated SNVs that underwent ≥90% single-nucleotide correction precision were observed (524 by editing with ABEs and 151 with CBEs), despite containing bystander C or A nucleotides within the editing window (FIG. 4C). Importantly, these SNVs could not be previously identified as likely candidates for high-precision single-nucleotide correction due to the presence of potential bystander substrate nucleotides, but were nonetheless predicted by BE-Hive with high accuracy in mES and HEK293T cells (R=0.78 to 0.92). BE-Hive predicted correction precisions were well-calibrated with observed correction precisions for all eight base editors in mouse and human cells (FIG. 4A and FIGS. 13E-13G): for example, sgRNA-targets with predicted correction precisions above 90% had an average observed correction precision of 91.8% with BE4, and analogously, predictions between 45-55% had an average observed correction precision of 48.4%, and predictions between 25-35% had an average correction precision of 27.1%. Thus, BE-Hive enables accurate apriori identification of targets amenable to highly precise single-nucleotide base editing despite the presence of bystander nucleotides.

When only a single C or A nucleotide is present in the editing window, prediction of single-nucleotide base editing precision may seem trivial. However, substantial variation in editing outcomes by CBEs and ABEs was observed even among these substrates. With only a single cytosine at position 6 in its window, BE4 single-nucleotide correction precision ranged from 0% to 100% at 157 sites with an average of 65% (FIG. 4D), demonstrating that at some sequence contexts, editing outside of the activity window can be at least as efficient as editing within the window. BE-Hive accurately predicted outcomes at target sites with a single editable nucleotide in the window, with R ranging from 0.92 to 0.94 for CBEs and 0.79 to 0.93 for ABEs. Similarly, editing outcomes varied substantially when exactly two editable C or A nucleotides were present at fixed protospacer positions. At 31 disease-associated SNVs positioned at C5 with a bystander C3 and no other cytosines (IUPAC code D) in positions 2-10, single-nucleotide correction precisions by BE4 ranging from 5.6% to 93% (FIG. 4E) were observed. Similarly, at 136 SNVs positioned at A6 with a bystander only at A8, single-nucleotide correction precisions by ABE ranged from near 0% to 99% (FIG. 4F). These data demonstrate that base editing precision is not dependent on position and number of editable nucleotides alone. Importantly, for both classes of target sequences, BE-Hive accurately predicted correction precisions with R=0.94 for BE4 and 0.71 for ABE.

These results reveal that single-nucleotide base editing relies on a complex relationship between the position of target and bystander nucleotides and base editor sequence preferences that cannot simply be derived from activity window and dinucleotide preference alone (see Supplementary Information for Example 1), but that can be accurately captured by machine learning. For example, a few SNVs at protospacer positions 5 and 7 achieved the highest correction precision of all CBEs by editing with the wide-window editor BE4-CP (FIG. 4G), even with additional cytosines present in its window. BE-Hive performed very strongly across CBEs at predicting correction precision at targets with at least one bystander C in each editor's activity window (FIGS. 4G-4H; BE-Hive R=0.91 and 0.96).

Taken together, the above results establish BE-Hive as an experimentally validated method for optimizing base editor choices to produce desired editing outcomes in mammalian cells—including those that cannot be predicted by inspection—with high precision, and to identify sites amenable to precise editing that would not otherwise be candidates for precision base editing.

### Target Sequence Features Partially Determine Rare CBE Outcomes

The occurrence of rare base editing outcomes varies by base editor, cell type, and target site. While cytosine transversion byproducts and indels that result from CBEs are thought to arise from abasic lesions produced by UNG-mediated removal of uracil (Komor et al., 2016), native motifs of UNG-mediated cytosine transition and transversions (WACT and WGCT, respectively) are weak predictors of CBE-editing outcomes (FIGS. 2D-2E and FIG. 11A; Supplementary Information for Example 1) (Pérez-Durán et al., 2012). An assessment of the contribution of sequence context in determining specific CBE-mediated cytosine conversion to G and A, its potential utility in editing disease-relevant SNVs, and the ability of BE-Hive to accurately predict these events were sought.

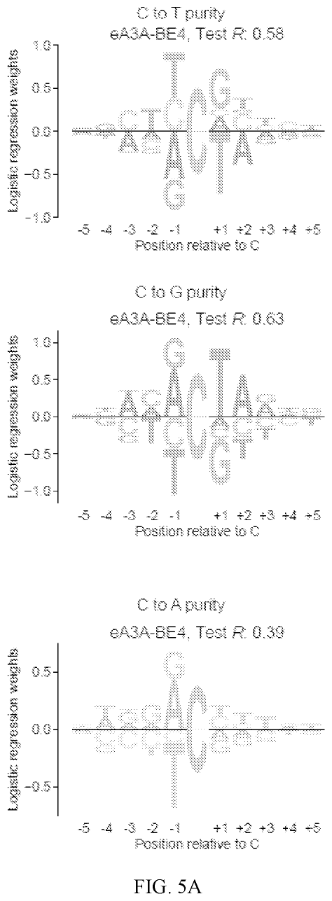

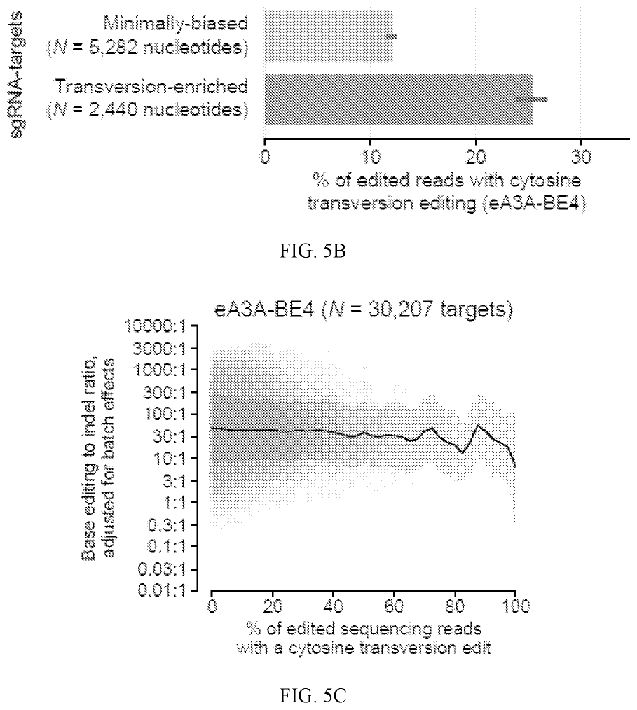

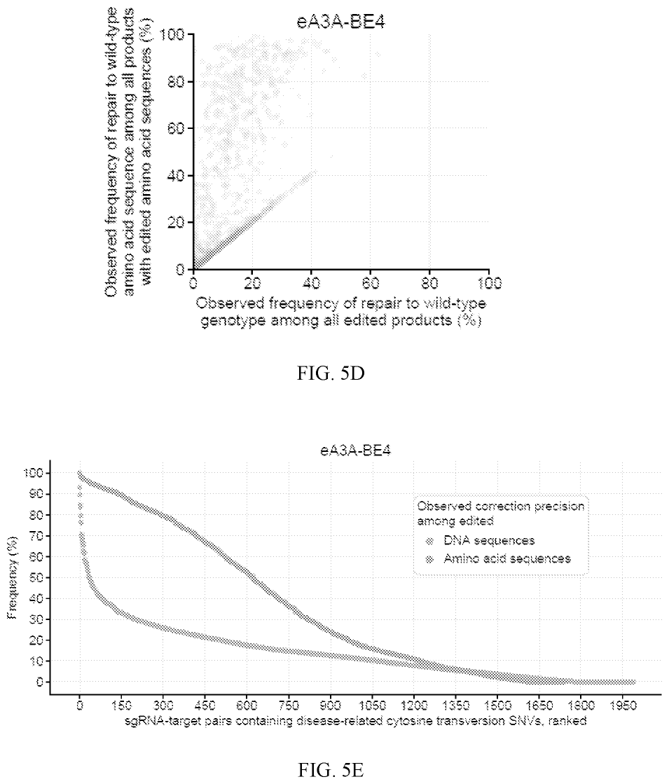

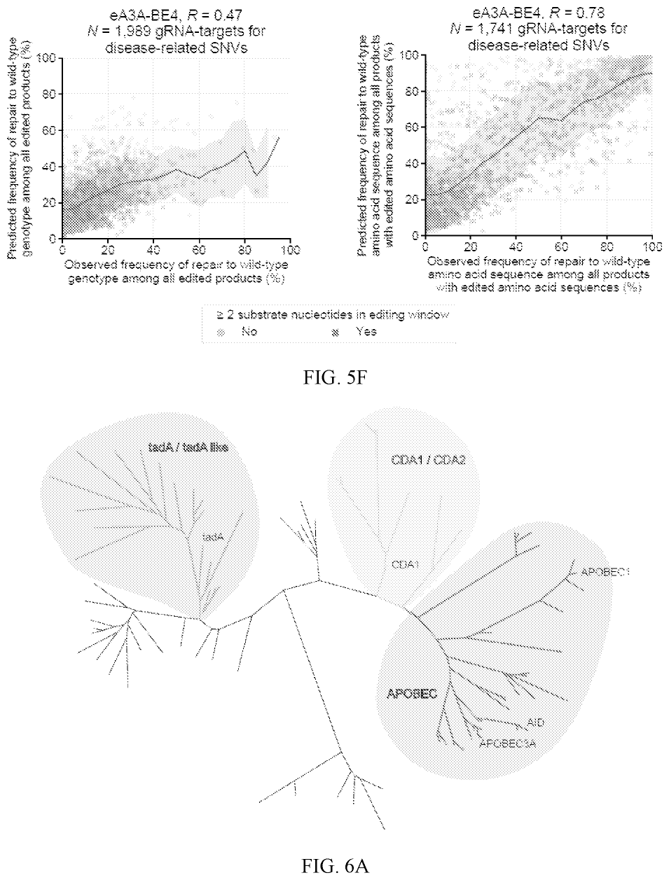

Whether sequence contexts predicted by BE-Hive to support CBE-mediated transversion are frequent in disease-relevant contexts was investigated. The search was focused on targets editable by eA3A-BE4 editing, which displayed the highest frequency of cytosine transversion byproducts in the library assay (FIGS. 1B-1I). Among 18,523 ClinVar and HGMD human disease-associated cytosine transversion variants, BE-Hive identified 2,090 unique alleles predicted to be predisposed to C⋅G-to-G⋅C conversion, and 289 alleles predisposed to C⋅G-to-A⋅T conversion by eA3A-BE4 and eA3A-BE4-NG editing. While an RCTA motif (test R=0.63) is predictive of C⋅G-to-G⋅C conversion, a looser and weaker RC motif (test R=0.39) is predicted to predispose sites to C⋅G-to-A⋅T outcomes (FIG. 5A). These findings suggest that sequence features not only affect the ratio of CBE-mediated cytosine transition versus transversion outcomes but may also determine the specific transversion product.

The significance of these sequence features was experimentally expressed using a library of 3,400 sgRNA-target pairs predicted to induce 8.5%-78% precise single-nucleotide C⋅G-to-G⋅C conversion and 400 sgRNA-target pairs to induce 5.9%-30% C⋅G-to-A⋅T conversion among edited outcomes by eA3A-BE4 and eA3A-BE4-NG editing, which was collectively named the “transversion-enriched SNV library”. Higher cytosine transversion purity in mES cells in this library was observed, averaging 25% by eA3A-BE4-NG, compared to 12% by eA3A-BE4 in the comprehensive context library (P=2.7×10⁻⁹³, Welch's T-test, N=2,440 versus 5,282 substrate nucleotides; FIG. 5B) and compared to approximately 3% on average across all other CBEs tested. These results indicate that BE-Hive learned sequence features that determine cytosine transversion outcomes of cytosine base editing.

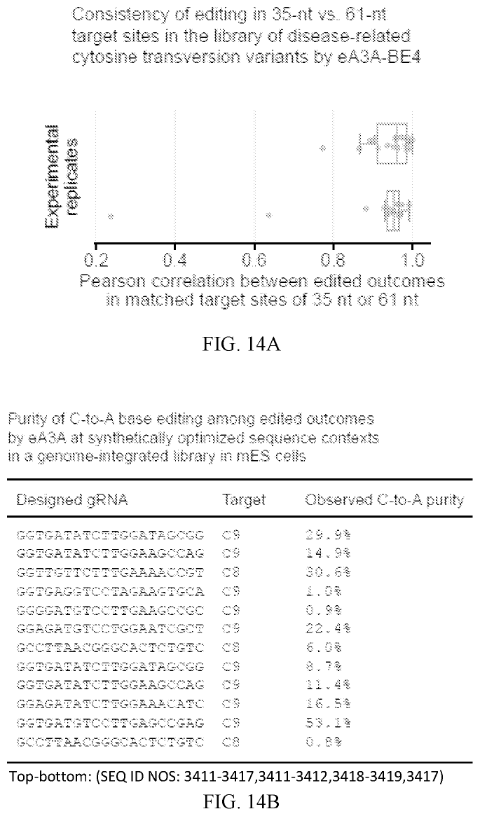

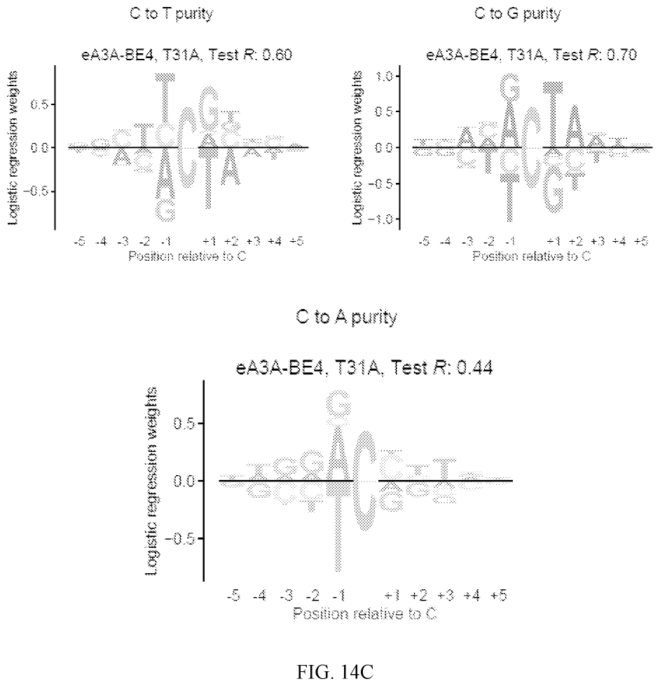

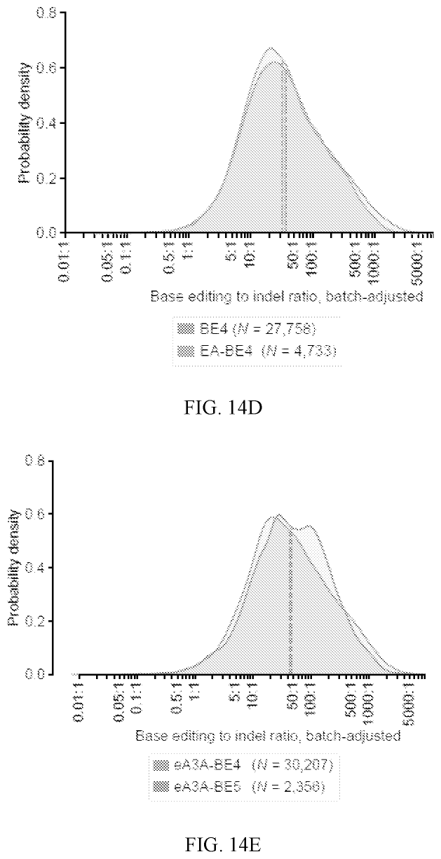

Among cytosine transversion outcomes, C⋅G rarely converts to an A⋅T (Imai et al., 2003). To investigate whether some contexts could support C⋅G-to-A⋅T conversion as the main product, BE-Hive was used to design 20 synthetic sequences optimized for this goal and observed a 4-fold elevated mean C⋅G-to-A⋅T editing purity of 16% among edited products, with a maximum of 53% (FIG. 14B), compared to the baseline average purity of 4.0% of edited outcomes across the comprehensive context library by eA3A-BE4 (P=0.0195, Welch's T-test, N=13,627 vs. 12 substrate cytosines in 12 target sequences). These data suggest that BE-Hive has learned sequence features that influence both types of cytosine transversion outcome at a given site.

Whether CBE-mediated cytosine transversions co-segregate with indels was explored, and no meaningful relationship was observed between cytosine transversion purity and BE:indel ratio by eA3A-BE4-NG editing (R=−0.02, P=0.2, N=4,320 target sites; FIG. 5C). These data suggest that the disease-associated sequence contexts predicted by BE-Hive to yield heightened transversion product purities enrich for specific resolution of abasic intermediates towards transversion edits, rather than merely increasing abasic site formation by promoting base excision that would increase the frequency of both outcomes.

### CBE-Mediated Correction of Transversion SNVs

Many SNVs in protein coding regions are known to cause human disease (Landrum et al., 2016; Stenson et al., 2014). For missense or nonsense variants, correction to the wild-type or a synonymous coding sequence can be sufficient to restore protein function. A correction of 121 disease-associated transversion SNVs was achieved in the transversion-enriched SNV library with ≥90% precision among edited amino acid sequences (≥90% amino acid precision) for C⋅G-to-G⋅C at 118 SNVs and for C⋅G-to-A⋅T at 3 SNVs (FIGS. 5D-5E). Importantly, BE-Hive accurately predicted amino acid precisions by eA3A-BE4-NG at these sites (R=0.78; FIG. 5F), enabling the correction of an entirely new class of point mutants not previously considered candidates for correction by CBEs. These included four distinct hemophilia A related alleles of factor VIII (F8), also known as anti-hemophilic factor (AHF), a disease that is considered a viable candidate for gene therapy approaches as only 1% restoration of plasma levels offers therapeutic benefit to patients (Doshi and Arruda, 2018). All four cytosine transversion alleles were corrected with 95% amino acid precision on average as predicted by BE-Hive (92% average; and above average editing efficiency (15% compared to 12% average editing across the transversion-enriched SNV library).

BE-Hive predicted the precise single-nucleotide correction of cytosine transversion SNVs with moderate accuracy of R=0.47 (FIG. 5F), indicating that the learned RCTA motif is an important but incomplete determinant of cytosine transversion purity. 33 unique disease-associated SNVs in which exact single-nucleotide correction by conversion of C⋅G to either G⋅C or A⋅T was the dominant editing outcome in ≥50% of edited reads was observed. The highest C⋅G-to-G⋅C correction precision achieved was 93% at a pathogenic mutation in the dystrophin gene (DMD), while the highest C⋅G-to-A⋅T correction precision was 28% for a pathogenic mutation in MutL homolog 1 (MLH1).

The above findings experimentally confirm BE-Hive predictive accuracy in identifying sequence determinants of CBE-mediated transversion outcomes, enabling the identification and correction of a previously unrecognized class of disease-relevant SNVs by cytosine transversion base editing.

### Mutations to Conserved APOBEC Residues Increase Rare Cytosine Transversions

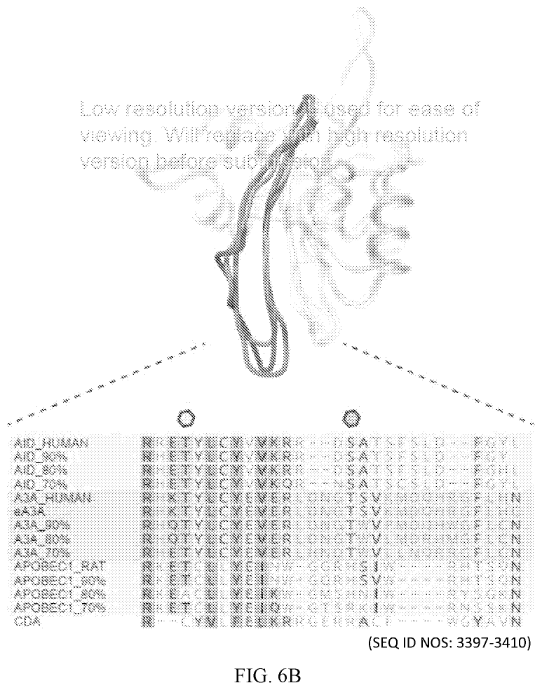

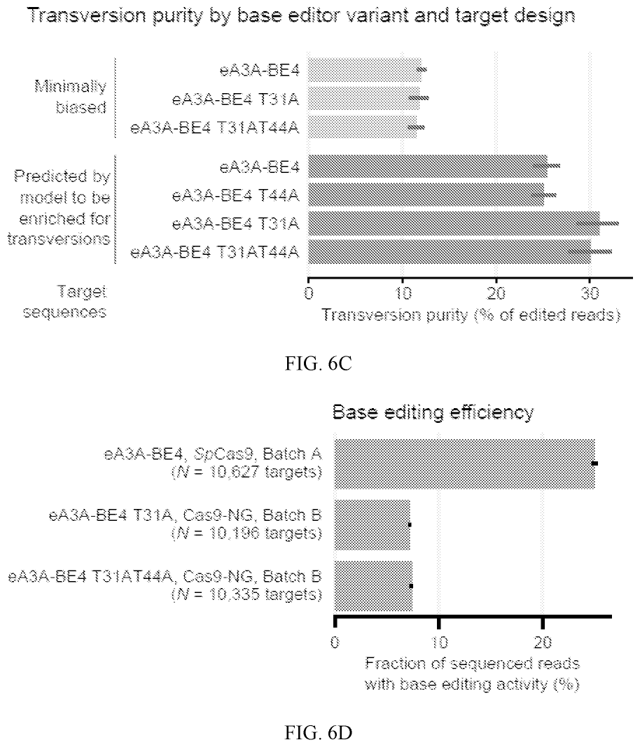

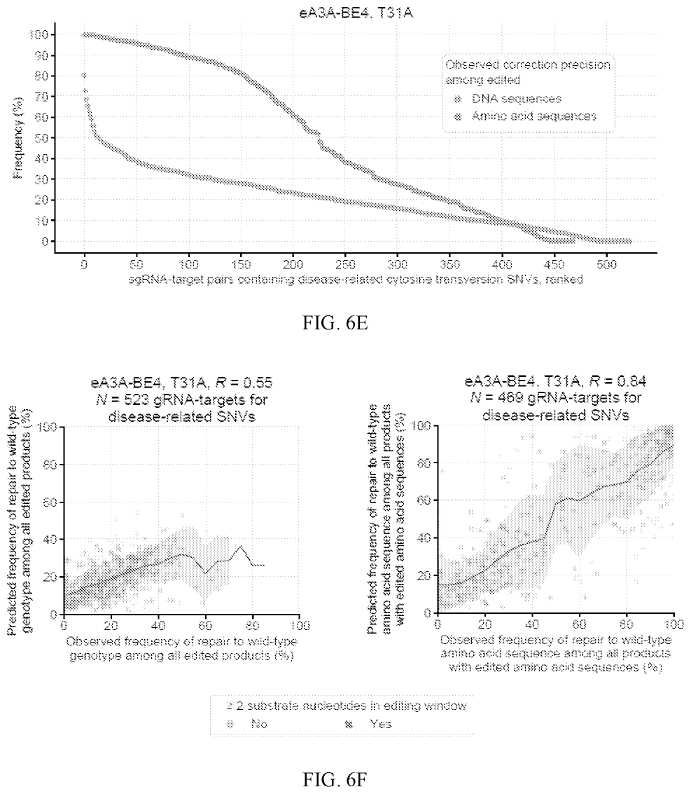

To dissect the role of CBEs in promoting rare editing outcomes, the means by which fused cytosine deaminases affect U⋅G mismatch repair was investigated (Supplemental Information to Example 1). Notably, CDA-BE4 yields transversion and indel base editing products at frequencies lower than that of other CBEs or what may be expected from its editing window size alone (FIGS. 1B-1I and 2E) (Komor et al., 2017). The DNA-mutating sea lamprey (Petromyzon marinus) derived CDA1 enzyme is evolutionarily more distant, and shares fewer conserved residues with, the mammalian APOBEC proteins assayed as CBEs (FIGS. 6A-6B).

The resolution of U⋅G to canonical or rare outcomes is mediated by endogenous DNA repair. The difference in CDA-BE4 outcomes relative to the trend among other CBEs may suggest that APOBEC family deaminases mediate interactions with DNA repair factors differently from CDA1. With the exception of AID, interactions between cytosine deaminases investigated here as components of CBEs and mammalian DNA-repair proteins have not extensively been studied (Adolph et al., 2017; Chaudhuri and Behan, 2004). In somatic hypermutation and immunoglobulin class-switching, phosphorylated residues S38 and T27 in AID are thought to play a role in determining repair outcomes of U⋅G mismatches (Basu et al., 2005; McBride et al., 2008; Pham et al., 2008; Yamane et al., 2011). These phosphorylation sites are not conserved in CDA1 but are widely conserved among mammalian APOBEC family members (FIG. 6B) (Blom et al., 2004), leading us to speculate that these protein domains may play a role in influencing editing outcomes of some CBEs.

Whether the mutation of conserved residues in APOBEC family members could affect partitioning of U⋅G mismatch repair outcomes was investigated. T31 in eA3A-BE4-NG, homologous to T27 in AID, was mutated to alanine (A), and an increase in transversion outcomes was observed in the transversion-enriched SNV library to 31%, compared to 25% by eA3A-BE4-NG (P=1.9×10⁵, Welch's T-test, N=2,440 versus 1,741 substrate nucleotides; FIG. 6C, and compared to approximately 3% on average across all other CBEs on the comprehensive context library. The T31A mutation did not meaningfully alter cytosine transversion motifs (FIG. 11A and FIG. 14C) or BE:indel ratios (46:1 compared to 45:1) relative to eA3A-BE4, though a reduction in editing efficiency was observed (FIG. 6D), consistent with reports on the T27A mutation in AID (Basu et al., 2005). In contrast, alanine mutation of T44, equivalent to S38 in AID, did not significantly affect editing outcomes (FIG. 6C). These results suggest that mutation of some conserved phosphorylated residues in CBE-fused APOBEC family members can affect the distribution of cytosine base editing outcomes.

Notably, the increase in transversion purity by eA3A-BE4-NG(T31A) was site dependent. While the mean transversion frequency in the comprehensive context library in mES cells was unchanged relative to eA3A-BE4, a 2.9-fold increase was observed in the fraction of alleles corrected with ≥90% amino acid precision by C⋅G-to-G⋅C or C⋅G-to-A⋅T editing of the transversion-enriched SNV library to 20% of assayed targets (FIG. 5E and FIG. 6E). These included two pathogenic G⋅C-to-C⋅G alleles of the low-density lipoprotein receptor gene (LDLR) that cause familial hypercholesterolemia; each was corrected back to wild-type with 100% and 99% precision among edited amino acid sequences. These data demonstrate that eA3A-BE4-NG(T31A) can increase cytosine transversion purity at disease-associated SNVs that support transversion outcomes. Collectively, these findings suggest that deaminases strongly affect the partitioning of U⋅G mismatch repair outcomes that arise from abasic lesions, establishing a new role for CBE deaminases beyond deamination activity alone.

Importantly, BE-Hive predictions of cytosine transversion outcomes were accurate, with R=0.84 for amino acid precision and R=0.55 for predicting genotype precision (FIG. 6F). Among SNVs identified by BE-Hive, 66 unique G⋅C-to-C⋅G coding mutations were corrected in 25 of the 59 genes identified as medically actionable by the American College of Medical Genetics (ACMG 59 genes) (Kalia et al., 2016) by editing with eA3A-BE4 variants, achieving ≥78% average amino acid precision (BE-Hive predicted average 74%). These findings demonstrate the utility of BE-Hive in designing base editing experiments for precision editing of clinically relevant targets that were not previously appreciated as likely candidates for CBE-mediated correction, by both canonical and non-canonical editing.

### Mutations to Conserved APOBEC Residues Improve Cytosine Transition Purity

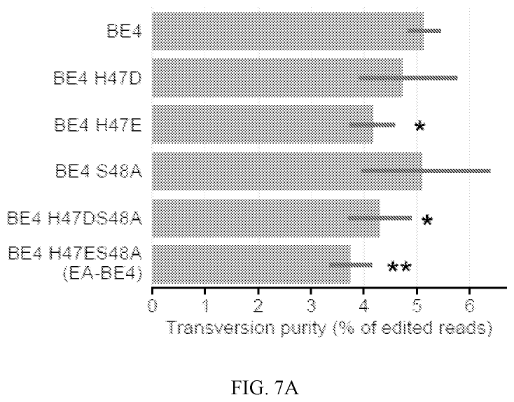

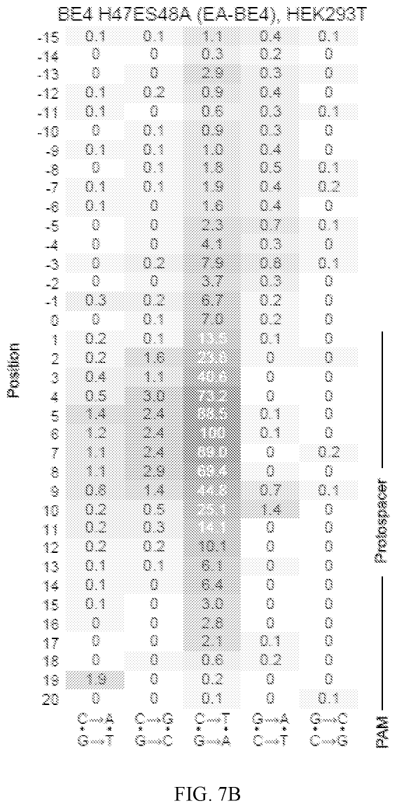

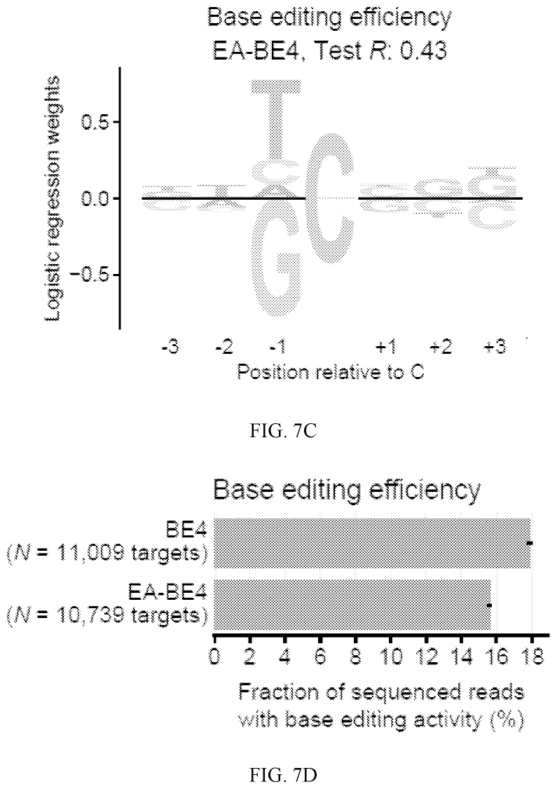

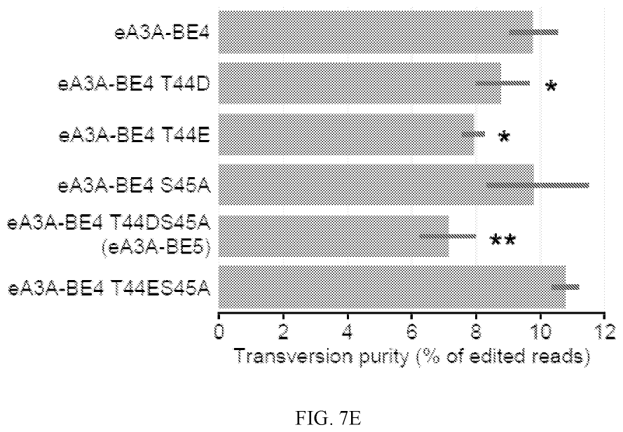

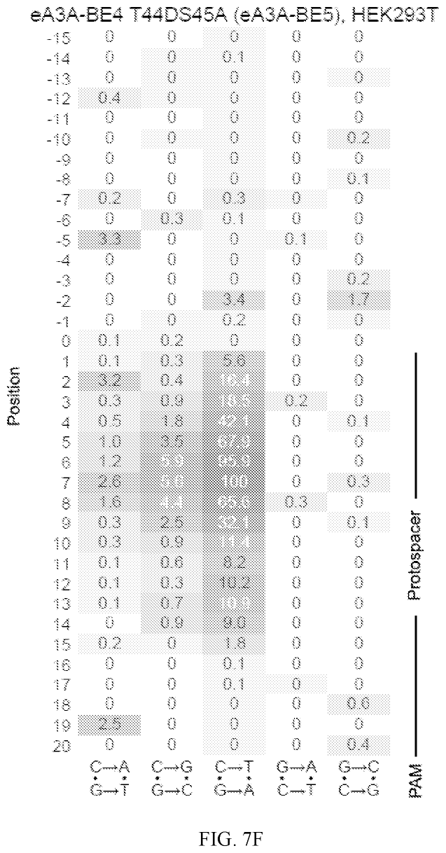

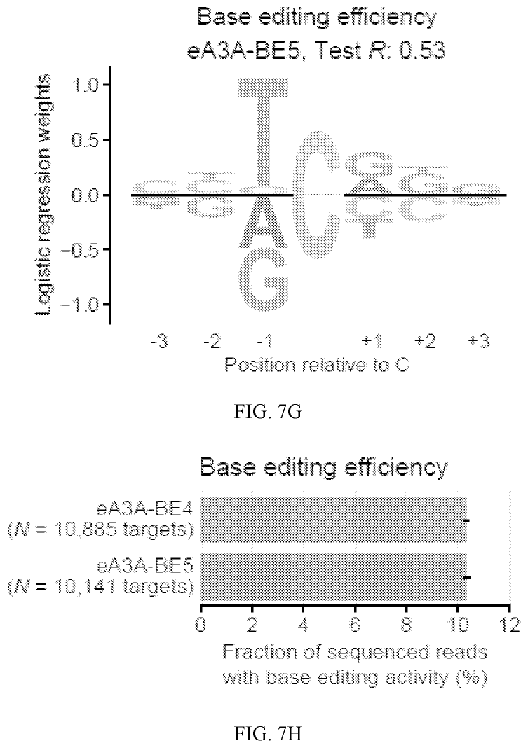

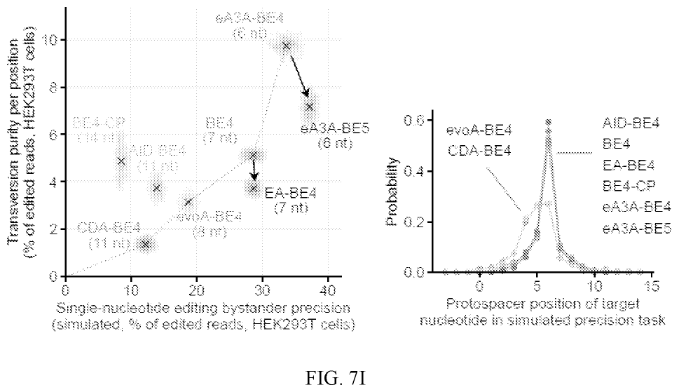

Given the observation that mutation of conserved residues in eA3A-BE4 can affect CBE outcomes, whether deaminase variants can decrease unintended transversion edits, and thereby increase desired C⋅G-to-T⋅A product purities, was investigated. Residue S38 in AID is a known PKA target (Basu et al., 2005), and computational analysis revealed this phosphorylation site is conserved (Blom et al., 2004). Phosphomimetic amino acid substitution to either aspartate (D) or glutamate (E) of APOBEC1 residue H47, equivalent to AID S38, was examined in BE4 (FIG. 6B). Cytosine transversion outcomes on the comprehensive context library in HEK293T cells was measured, and indeed a reduction in transversion byproducts from 5.1% average by BE4 editing, to 4.7% by H47D (P=0.41) and 4.2% by H47E variants (P=1.3×10⁻⁴, Welch's T-test; FIG. 7A) was observed.

Mutation of the adjacent conserved residue S48 to alanine further reduced transversion byproducts resulting from these variants, down to 3.7% for BE4(H47E+S48A) (FIG. 7A). This variant (EA-BE4) reduced transversion product purity by 27% on average compared to BE4 (95% CI: 18-35% reduction, P=1.5×10⁻⁸, Welch's T-test, N=3,636 and 1,208 substrate nucleotides), while maintaining a similar editing window, editing sequence preference, and disequilibrium score (FIGS. 7B-7C), but with a small loss in editing efficiency (averaging 16%, compared to 18% in BE4 in the same batch; FIG. 7D) and a slight shift in BE:indel ratio (32:1 with IQR=12:1 to 85:1, compared to 36:1 with IQR=12:1 to 100:1 for BE4; FIG. 14D).

Next, the same changes were introduced to equivalent residues in eA3A-BE4 to investigate whether the effect of these mutations is generalizable among APOBEC family members. In HEK293T cells, D and E substitution of T44, equivalent to S38 in AID, reduced undesired transversion edits from 9.8%, to 8.8% (P=0.06) and 7.9% (P=4.2×10⁻⁷), respectively (FIG. 7E). Alanine substitution of the adjacent conserved S45 residue alone did not have a significant effect, but the combination of T44D+S45A further lowered transversion purity to mean 7.1%, reduced by 27% compared to canonical eA3A-BE4 editing (95% CI: 17-36% reduction; P=1.0×10⁻⁶, Welch's T-test, N=1,837 and 685 substrate nucleotides). Identical editing efficiency was observed in the same experimental batch by the T44D+S45A variant and eA3A-BE4 and a mildly elevated geometric mean BE:indel ratio (46:1 compared to 43:1, respectively) with no effect on editing window, sequence preference, or disequilibrium score (FIGS. 7F-7H and FIG. 14E). Furthermore, a minor improvement in single-nucleotide editing bystander precision of 15% (38% in eA3A-BE4(T44D+S45A) was noted, relative to 33% in eA3A-BE4, FIG. 7I), achieving the highest single-nucleotide editing precision of all CBEs tested here. No apparent downsides were observed to using eA3A-BE4(T44D+S45A) relative to eA3A-BE4 among the many CBE characteristics examined across thousands of target sites described herein, therefore this eA3A base editor variant was named eA3A-BE5.

Collectively, these data demonstrate that mutation of conserved phosphorylation targets in APOBEC family members can affect cytosine transversion byproducts of multiple cytosine base editors. While CDA-BE4 and evoA-BE4 demonstrate higher C⋅G-to-T⋅A purity than the EA-BE4 or eA3A-BE5, CDA-BE4 and evoA-BE4 have substantially larger editing windows and therefore offer low bystander precision, often making them less suited for precision editing applications (FIG. 7I). The optimal base editor choice for precision editing lies on a Pareto frontier that balances the relative risk of bystander versus transversion edits. EA-BE4 and eA3A-BE5 represent novel optimal CBEs that lay beyond the Pareto frontier defined by established base editors and provide narrow-window base editing with minimal cytosine transversion editing activity.

### Supplemental Information to Example 1

### Approach to Systematically Characterize Base Editing Activity

The assay's high sensitivity and large, minimally biased set of sequences enabled us to describe base editing windows with greater accuracy and generality. The comprehensive characterization library included all possible 6-mers surrounding a substrate A or C nucleotide at protospacer position 6, and all possible 5-mers spanning positions −1 to 13. Within this design series, a particular target sequence can contain more than one such 5-mer, enabling the compression of 11×45=11,264 designs into 2,496 sgRNA-target pairs.

Data across the library was collected to sensitively identify editing events with frequencies below 0.1%. This sensitivity was possible because a mutation event confidently identified at, for example, 10% frequency in one out of 1,000 target sites occurs at 0.01% frequency in aggregate. Maintaining a threshold of 50% or greater of their maximum frequency, windows of 4-8 for BE4 and 3-8 for evoA-BE4 were observed, consistent with previous reports (Komor et al., 2017a; Thuronyi et al., 2019), while BE4-CP ranges from 4-13 though it was previously estimated as 4-11 (Huang et al., 2019b). Similarly, at this 50% threshold, editing windows from position 5-7 for ABE and 4-9 for ABE-CP were observed, previously reported as position 4-7 and 4-12, respectively (Gaudelli et al., 2017). Editing windows at a 50% maximum activity were observed at threshold ranging position 1-10 for AID-BE4, 0-8 for CDA-BE4, and 5-8 for eA3A, compared to 3-7, 2-8, and 4-8, respectively (Gehrke et al., 2018; Nishida et al., 2016; Rees and Liu, 2018; Ren et al., 2018).

The definition of the typical editing window was broadened to a threshold of ≥30% to better include all positions that can undergo substantial base editing, though moderate base editing activity is still expected to occur outside this window as well. Across the comprehensive context library, ABE is a narrow window editor with typical editing activity spanning protospacer positions 4-8, while ABE-CP is a wide window editor that typically edits positions 3-11. BE4 has a narrow editing window from position 3-9, which was slightly increased by protein evolution in evoA-BE4, a narrow window editor ranging from 2-9. BE4-CP, AID-BE4 and CDA-BE4 are all wide window editors at 2-15, 1-11, and −1-9 respectively. eA3A-BE4 is a narrow window editor, with typical editing activity between protospacer positions 4-9, though a unique bimodal editing profile was noted with an additional peak in C⋅G-to-T⋅A editing at protospacer position 13 to up to 18% relative to eA3A-BE4's maximum positional editing frequency.

### Sequence-Activity Relationships for Common Base Editing Outcomes

While deaminase-specific sequence preferences have been reported to affect nucleotide conversion efficiencies of some base editors (Beale et al., 2004; Komor et al., 2016; Liu et al., 2018), sequence-activity relationships of base editors have not been characterized in depth. Resolution of base editing heteroduplex DNA intermediates containing deoxyuridine or deoxyinosine to a permanent edited product involves DNA repair pathways such as mismatch repair (MMR) and base excision repair (BER) (Pérez-Durán et al., 2012) that can also be influenced by local sequence context (Fishel, 2015; Jiricny, 2006; Mazurek et al., 2009). Base editing outcomes thus depend on target sequence in many potentially complex ways (Rees and Liu, 2018).

Sequence motifs were generated for various base editing activities by using logistic regression to predict activity from target sequence context and depict the learned weights as a sequence logo. The sign and weight of nucleotides in the logo depicts their contribution to activity; a weight of zero would indicate no change from the mean. To understand the relevance and strength of learned motifs, it is crucial to consider the motif's performance at predicting activity in sequences that were not used for training the motif models (held-out data), which were reported as Pearson's R or area under the receiver operator curve (AUC) for regression or classification tasks respectively.

First, sequence motifs were calculated for the efficiency of canonical base editing activity in which CBEs convert C⋅G to T⋅A and ABEs convert A⋅T to G⋅C. Motifs were obtained for each editor at ≥7,091 unique substrate nucleotides in their editing windows at ≥5,292 target sequences (FIG. 2C). Target sequence motifs were virtually identical to motifs calculated from the subset of the comprehensive context library with all 6-mers surrounding either C6 or A6 (FIGS. 10A-10B) and were consistent across cell types and biological replicates (FIG. 10C). These findings identify sequence context as an important determinant of editing activity across all editors with the exception of CDA-BE4, for which only 5.3% of the variance in editing efficiency is explained by target motifs in held-out sequences, compared to 15-32% on average across all other base editors.

### Deaminase and Sequence Context Affect Editing of Proximal Substrate Nucleotides

Deaminase enzymes and base editors tested here have been described as having varying degrees of processivity, the ability to sequentially catalyze multiple base conversions without releasing the target DNA (Gaudelli et al., 2017; Komor et al., 2016a; Love et al., 2012; Nishida et al., 2016; Pham et al., 2003). rAPOBEC1 CBEs such as BE4 and ABE base editors have been described as processive, while CDA-BE4 and eA3A-BE4 are thought not to be processive. Base editing processivity may be reflected in equilibrium scores, the ratio between observed frequency of two substrate nucleotides in a single substrate both being editing and the expected frequency of both nucleotides being edited together assuming statistical independence. Values above one indicate a preference for editing both or neither nucleotide over having only one or the other edited, consistent with processive base editing. Disequilibrium scores were calculated for the eight CBEs and ABEs using data from 614 to 4,796 pairs of substrate nucleotides in the editing windows of 390 to 1,413 target sequences in the comprehensive context library.

From this analysis, disequilibrium scores of 1.04 to 1.23 were observed across all CBEs, and 0.86 for ABE and 0.73 ABE-CP on average, FIG. 3F and FIGS. 13C-13D), contrary to prior observations demonstrating positive processivity of late-stage ABEs (Gaudelli et al., 2017). It was noted that disequilibrium scores calculated in this manner are unavoidably confounded by local sequence context preferences, such as ABEs dislike of AA contexts. While this model predicts that the disequilibrium scores for ABEs should increase for non-sequential adenines, only low levels of disequilibrium score increase were observed for ABE and ABE-CP at substrate nucleotides spaced more than one nucleotide apart.

Interestingly, it was observed that sequence context contributes more strongly to disequilibrium scores than the choice of deaminase. Many pairs of substrate nucleotides were observed with disequilibrium scores both >1 and <1 among different tested base editors. Among CBEs, eA3A-BE4 was particularly susceptible to sequence context, and demonstrated the greatest disequilibrium score of narrow-window editors in a sequence-dependent manner. Mild to no change in disequilibrium score was observed for most base editors as the substrate nucleotide pair distance varied from 1 to 8 bp apart.

Together, these data demonstrate that processive action of base editor deaminases at on-target sites, measured as joint editing probability, are a combined function of deaminase enzyme, activity range, and sequence context.

### Base Editing Model Design

A model to inform the design of base editing experiments including all possible choices of base editors, sgRNAs, and targets to enable a desired phenotype (FIG. 3A) was sought. Such a method should flexibly support user-specified definitions of desirable and undesirable editing outcomes: for example, in many base editing applications, “bystander editing” of non-target C or A nucleotides located near the target C or A are silent in the context of the translated amino acid sequence, yielding a multitude of desirable genotype edits. The design method should consider editing efficiency, sequence preferences for various editing outcomes and likelihood of bystander edits, each of which vary by base editor. To achieve these goals, two machine learning models were trained. The “editing efficiency model” takes a user-provided target sequence and base editor as input and uses gradient-boosted regression trees to predict an editing efficiency z-score which can be interpreted into a predicted fraction of sequenced reads containing base editing activity. The “bystander editing model” takes a user-provided target sequence and base editor as input and uses a deep conditional autoregressive model to predict the frequency of combinations of base editing outcomes at all substrate nucleotides among edited reads. For both model types, distinct models were trained on data from the library assay for each editor and cell type.

The relationship between sequence and base editing efficiency was investigated using the comprehensive context library across two biological replicates in each of three cell types. Sequence motif models were trained, and it was found that the learned motifs (FIG. 12E) resemble a combination of each editor's single-nucleotide sequence motif and activity window. Preferences for purines at position 20 related to sgRNA loading into Cas9 (Wang et al., 2014) and for G at position 0 were observed, indicating that 21 nt spacers that were extended with a 5′ G for the purpose of U6 promoter expression enable more efficient editing when all 21 nucleotides are complementary to the target than when the 5′ G is a mismatch, similar to observations in high-fidelity Cas9-variants (Kleinstiver et al., 2016).

To predict bystander base editing patterns, a deep conditional autoregressive model was designed (Van Den Oord et al., 2016) that uses an input target sequence surrounding a protospacer and PAM to output a frequency distribution on combinations of base editing outcomes (FIG. 3D), and trained the model on data from up to 10,638 sgRNA-target pairs in the comprehensive context library which were randomly split in an 8:1:1 ratio into training, validation, and test data sets. The model predicts all nucleotide substitutions from protospacer positions −10 to 20. The model learns sequence motifs with higher-order interactions by providing each substrate nucleotide and its surrounding nucleotides to a deep neural network which were referred to as an “encoder”. This series of encodings are decoded one by one using a “decoder” deep neural network. For each encoding (representing a substrate nucleotide), the decoder outputs a distribution of base editing outcomes. The decoder acts autoregressively, meaning it decodes an encoding while using all previously decoded outputs in the series as input.

To flexibly model editing profiles with any shape, a learned positional bias towards producing an unedited outcome was introduced. Importantly, the model can learn to capture any possible distribution of editing outcomes, and thus can learn the editing patterns of any base editor from sufficient editing outcome data. An architecture search, ablation analysis, and comparisons to baseline methods (STAR methods) were performed, and it was concluded that the autoregressive design and using a high-capacity decoder were important for predictive performance. Across the six CBEs and two ABEs tested here, the bystander model performed strongly at predicting the frequencies of bystander editing patterns, achieving a median R=0.86-0.99 on ≥606 held-out target sequences in mES cells (FIG. 3E and FIGS. 12H-12I). The model retained strong performance even at target sites with many substrate nucleotides (FIGS. 13A-13B). The large variance in disequilibrium scores of base editors (FIG. 3F and FIGS. 13C-13D; Supplementary Information) was accurately predicted by the bystander model (R=0.71 and 0.74 for BE4 and eA3A-BE4), demonstrating that it has learned higher-order conditional editing probabilities.

The editing efficiency and bystander editing models were collectively named “BE-Hive”. Using target sequence as input alone, BE-Hive estimates base editing efficiency and outcomes at the single-nucleotide and coding-level. BE-Hive represents the first tool for designing base editing experiments that comprehensively considers on-target editing efficiency, deaminase and sequence related preferences for various editing outcomes, and the likelihood of bystander edits to distinguish targets that are amenable to high-precision single-nucleotide editing and coding-sequence correction using a variety of established base editors (FIG. 3G).

Characterization of Indels Resulting from Base Editing

To date, indels resulting from base editing activity have remained poorly characterized. During cytosine base editing, rare indels may result from DNA nicking by the HNH nuclease domain on the protospacer-bound DNA strand and abasic site generation at deaminated cytosines through UNG-mediated excision of uracil, which can convert to a DNA strand break spontaneously or during base excision repair. During adenine base editing, deoxyinosine can be recognized by enzymes such as alkyl-adenine DNA glycosylase (AAG) and excised to facilitate base excision repair (Lau et al., 2000), although, AAG has been reported to have little activity on ssDNA (Hitchcock et al., 2004; Saparbaev and Laval, 1994). ABE-mediated adenine deamination products therefore may convert to abasic sites less frequently than CBE deamination products, which may explain why indels occur less frequently than in cytosine base editing (Gaudelli et al., 2017).

In order to sensitively identify indel activity, data was surveyed at a subset of target sequences (N>19,925) per editor in HEK293T cells, U2OS cells, and mESC cells with high read count, and adjusted for batch effects with two-way ANOVA. In untreated library cells, 1-bp variations from designed sequences were observed, presumably attributable to errors in synthesis, PCR amplification, and HTS. This noise was corrected for by comparing treatment library data to untreated library data and data from endogenous contexts (STAR methods, FIGS. 11B-11E). Among cell types, a 1.2-fold increase in BE:indel ratio in HEK293T cells, and 2.1-fold increase in U2OS cells relative to mESCs was observed, although neither of these differences were statistically significant (FIG. 11D). These results suggest a minor role for cell type differences in affecting the ratio of BE:indel outcomes.

Wide-window editors induced indels at a lower relative frequency than narrow-window editors in both CBEs and ABEs (FIG. 2G and FIG. 11E). An average geometric mean BE:indel ratio of 129:1 for ABE and 37:1 in narrow-window CBEs, and 166:1 for ABE-CP and 46:1 in wide-window CBEs, was detected representing typical indel frequencies of 0.2% and 0.5% in ABEs and CBEs, respectively. A weak relationship was observed between target sequence and frequency of indels resulting from base editing reflected by low replicate consistency of BE:indel ratios at matched target sites (IQR R=0.13 to 0.29 across editors in mES cells, P<3.8×10-3). Overall, the comprehensive characterization of BE:indel ratios confirmed the rarity of undesired indel events by base editors.

The indel outcome analysis revealed a characteristic profile of indels that result from base editing. Deletions resulting from cytosine base editing were most frequently centered around the PAM-proximal Cas9 HNH domain's nick locations preceding position 18, the PAM-distal deamination peak position for a given editor (often position 6), or spanning these two sites resulting in a peak in outcome frequency at ˜12 bp deletions (FIG. 2H and FIG. 12A), consistent with the understanding of the processes that give rise to indel events. However, the peak position of PAM-distal deletions that arise from deamination events did not always mirror the distribution of deamination activity in the editing window of all editors. While the BE4-CP editing window ranges from position 2-15 with peak editing at the central position 8, indels resulting from cytosine deamination were offset towards the PAM. Interestingly, cytosine transversion mutations induced by BE4-CP are likewise shifted in their location towards the PAM (FIGS. 1B-1I), consistent with a model in which both indel formation and C⋅G-to-G⋅C and C⋅G-to-A⋅T mutations arise from repair of abasic lesions following uracil excision.

The rare insertion outcomes from base editing are distinct from typical Cas9 nuclease-induced insertion products (Shen et al., 2018). Base editor-mediated insertions occurred primarily at the Cas9 HNH nick for both ABEs and CBEs, and were separable into three classes that occurred at approximately equal frequency: first, duplications of a single nucleotide, comprising 25-35% of insertions; second, a single repeat of two or more nucleotides from the native sequence context at 33-34%; and third, insertions of two or more nucleotides that do not correspond to duplications of the native sequence context, comprising 30-36% of insertions (FIG. 2I and FIG. 12B). In Cas9-genome editing, insertion genotypes are heavily dominated by 1-bp insertion products that are frequently a duplication of the nucleotide immediately 5′ of the double-strand break (DSB) site (Allen et al., 2019; Shen et al., 2018). Base editor-induced insertions appeared to be consistent with Cas9-nuclease insertion mutations in that they often duplicate the sequence 5′ of the HNH nick, though more typically consist of longer duplicated regions. Cas9 DSB-mediated 1-bp insertions are thought to arise from occasional staggered cutting which causes a 3′-overhang that is filled in by DNA-polymerase and ligated by non-homologous end joining (Lemos et al., 2018; Richardson et al., 2016; Shou et al., 2018; Zuo and Liu, 2016). Although this same mechanism cannot explain insertions that arise from base editing, it is tempting to speculate that longer 3′-overhangs resulting from base editing-induced abasic lesions and HNH nick activity may similarly contribute to insertion outcomes.

Cytosine transversion outcomes of base editing also arise from UNG-mediated abasic sites and were enriched at RCTA motifs (FIGS. 2D-2E); however, strong sequence determinants of indels that result from base editing were not observed. Sequence motifs were trained to predict BE:indel ratio from target sequence and identified a minor association of indels with adenine and thymine relative to cytosine and guanine (FIG. 12C). Overall these motifs performed weakly, explaining only 1-7% of the variation in BE:indel ratios in held-out sequences (P<7.0×10⁻³¹). Indels resulting from base editing may depend on the Cas9 component. A mild improvement in BE:indel ratio by base editing with NG-fused eA3A-BE4 overall (45:1) was noted, relative to eA3A-BE4 (43:1). The engineered Cas9-NG is reported to have lower activity than wild-type SpCas9 protein, similar to high-fidelity Cas9 variants that have reduced binding strength relative to wild-type Cas9, which may underlie this variability (Nishimasu et al., 2018).

These analyses provide the first comprehensive characterization of indels that result from base editing. The relative rarity of indels resulting from base editing by ABEs and CBEs was confirmed, and observed a minimal role for cell type, sequence context, and Cas9 component in determining their frequency. A characteristic profile of indels that result from base editing that is consistent with a model based on HNH-nicking and abasic site generation following deamination was discovered. Collectively, these findings suggest that rare base editor-induced indels may arise through similar, yet distinct mechanisms from Cas9 nuclease-induced indels.

### Model-Guided Design for Precise Base Editing Correction of Pathogenic Alleles

Optimal base editor choice for induction of a desired edit depends on sequence preferences and base editor position with respect to the substrate nucleotide. An increase in the number of editable nucleotides exponentially expands the combinatorial space of potential outcomes at a given target, further complicating experimental design for precision editing applications. Across the six CBEs and two ABEs tested here, BE-Hive performed strongly with a median R=0.86-0.99 on 606 or more held-out target sequences (FIG. 3E). Mild reductions were observed in performance with increasing numbers of proximal substrate nucleotides and editor window size, achieving a median R=0.98 and R=0.90 at held-out target sites with two and five substrate nucleotides in positions 1-12, respectively (FIG. 4B).

The ways in which sequence composition affects single-nucleotide editing precision of ABEs and CBEs was unvestigated by considering subsets of SNP alleles in which the umber and position of substrate nucleotides in the editing window was controlled. For example, BE4 editing activity at 31 disease-related SNPs was investigated with a fixed cytosine at positions 3 and 5 with no other cytosines (IUPAC code D) in positions 2-10 (C3 and C5 mask) and observed a large amount of variation in single-nucleotide correction precision ranging from 5.6% to 93%, as predicted by BE-Hive (R=0.94, FIG. 4E). ABE demonstrated similar variability; for example, in editing of 136 disease-related alleles in the ABE precision editing SNP library in mES cells masked on A6 and A8, single-nucleotide correction precision ranging from 0% to 99% was observed, as predicted by BE-Hive (R=0.71, FIG. 4F). These analyses affirm that single-nucleotide precision is factor to more than the number and activity window position of substrate nucleotides. Sequence determinants that may appear relatively weak at single substrate nucleotides can combine into stronger sequence determinants when considering combinations of editing events.

Differences in base editor sequence preference result in variability in precision editing of target sites with multiple substrates (FIGS. 4G-4H). To illustrate this, eA3A-BE4 and BE4 editing in the CBE precision editing SNP library in mES cells was compared at two C7 SNP alleles—one for tetrameric protein transthyretin gene (TTR) involved in transthyretin amyloidosis (OMIM 105210), and one in the transmembrane protein 127 gene (TMEM127) related to pheochromocytoma (OMIM 171300)—where C4 and C7 are the only cytosines among positions 2-10. Single-nucleotide correction precision of 74% in TTR and 16% at TMEM127 by BE4 editing was observed, while eA3A-BE4 corrected both alleles at 91% and 90% precision, respectively. BE4's relative dislike of the GC motif at C4 compared to the AC motif at C7 may explain the high precision achieved in TTR editing and the lower precision in TMEM127 where both target and bystander nucleotide share the disfavored GC motif, however, eA3A-BE4 disfavors both these dinucleotide motifs equally and induced high precision edits in both alleles. The variability in precision editing is therefore dependent, but not fully explained by the deaminase dinucleotide preferences described in the literature, but is accurately captured by BE-Hive (R=0.96). While C4 and C7 both lie within the canonical editing window of eA3A-BE4, the average editing efficiency at position 7 is nearly double that of position 4. This finding agrees with, though is disproportionate to the heavy bias for precise editing of C7 in both TTR and TMEM.

Moreover, vastly different editing precision outcomes were observed even at sites with identical dinucleotide motifs and substrate position. In the myosin heavy chain beta gene (MYH7) SNP allele related to cardiac disease (Tajsharghi et al., 2003), and the glutamate ionotropic receptor NMDA type subunit 2B gene (GRIN2B) SNP allele related to a number of neurodevelopmental disorders (Hu et al., 2016), the target cytosine at position 7 lies within the disfavored AC context and the position 4 bystander cytosine is preceded by T, yet editing precision of C7 varied from 28% at MYH7 to 0% at GRIN2B. These data suggest that precision base editing relies on a complex relationship between the position of target and bystander nucleotides and base editor sequence preference that is not easily interpreted from window and dinucleotide preference alone.

In some cases, optimal base editor choice can even be counterintuitive. For example, at three targets with a pathogenic SNP at positions C5 or C7—the fibroblast growth factor receptor 1 gene (FGFR1) underlying Kallman syndrome (Dode et al., 2003), the growth differentiation factor 1 gene (GDF1) related to congenital heart defects (OMIM 613854), and in the polycystin 1 gene (PKD1) related to polycystic kidney disease—BE4-CP had higher genotype correction precision than any other CBE, even when additional cytosines were present within its wide editing window.

Bystander mutations at on-target sites may be innocuous [symbol] for example when they induce a silent mutation in a protein-coding gene, which is estimated to occur with 47% probability for CBEs with a 5-nt window and 38% for ABEs with a 4-nt window (Rees and Liu, 2018) [symbol] or deleterious if they introduce unwanted functional changes in protein coding or regulatory regions. Functionality was added to BE-Hive to predict changes to amino acid sequences following base editing to help further distinguish favored from unfavored edited outcomes (FIG. 3A and FIG. 3G).

### Sequence Features Partially Determine Rare CBE-Outcomes

The occurrence of rare base editing outcomes varies by base editor, cell type, and target site. Both cytosine transversion byproducts and indels that result from CBEs are thought to arise from abasic lesions induced by UNG (Komor et al., 2016). The sequence motif describing uracil excision from double-stranded DNA (dsDNA) by UNG-family members in vitro is approximated as WCAW, and in the context of somatic hypermutation (SHM) UNG demonstrates a preference for inducing transversions at deaminated WGCT and transitions at WACT motifs (Pérez-Durán et al., 2012). These motifs differ substantially from the RCTA motif observed in the analysis to enrich for cytosine transversion events (FIGS. 2D-2E and FIG. 11A). Thus, native UNG preferences are weak predictors of cytosine transversion outcomes that result from CBE editing. An assessment of the contribution of sequence context in determining specific CBE-mediated cytosine conversion to G and A, its potential utility in editing disease-relevant SNVs, and the ability of BE-Hive to accurately predict these events, were sought.

It was found that while an RCTA motif (test R=0.63) is predictive of C⋅G-to-G⋅C conversion, a looser and weaker RC motif (test R=0.39) is predicted to predispose sites to C⋅G-to-A⋅T outcomes (FIG. 5A). These findings suggest that sequence features not only affect the ratio of CBE-mediated cytosine transition versus transversion outcomes but may also determine the specific transversion product. The significance of these sequence features was experimentally assessed using a library of 3,400 sgRNA-target pairs predicted to induce 8.5%-78% precise single-nucleotide C⋅G-to-G⋅C conversion, and 400 sgRNA-target pairs to induce 5.9%-30% C⋅G-to-A⋅T conversion among edited outcomes by eA3A-BE4 and eA3A-BE4-NG editing, which was collectively named the “transversion-enriched SNV library”. For technical reasons, the library contained 35-nt and 61-nt target sites, but base editing outcomes were highly consistent between target sites of differing length that represented the same sequence contexts (median R=0.96; FIG. 14A). Higher cytosine transversion purity in mES cells was observed in this library, averaging 25% by eA3A-BE4-NG, compared to 12% by eA3A-BE4 in the comprehensive context library (P=2.7×10-93, Welch's T-test, N=2,440 versus 5,282 substrate nucleotides; FIG. 5B) and compared to approximately 3% on average across all other CBEs tested. These results indicate that BE-Hive learned sequence features that determine cytosine transversion outcomes of cytosine base editing.

Whether CBE-mediated cytosine transversions co-segregate with indels and observed no meaningful relationship between cytosine transversion purity and BE:indel ratio by eA3A-BE4-NG editing as also explored (R=−0.02, P=0.2, N=4,320 target sites; FIG. 5C). These data suggest that sequence contexts with enriched transversion product purities enrich for specific resolution of abasic intermediates towards transversion edits, rather than merely increasing abasic site formation by promoting base excision that would increase the frequency of both outcomes.

Taken together, these data establish the importance of sequence context in determining both the frequency and the identity of repair products that arise from abasic intermediates of cytosine base editing. Target sequences predicted by BE-Hive greatly enriched C⋅G-to-G⋅C and C⋅G-to-A⋅T outcomes from cytosine base editing of disease-associated alleles without increasing indels.

### Deaminase Enzymes Partially Determine Rare CBE Repair Outcomes

Indels and transversions have previously been noted as byproducts of CBE editing, however, factors that determine their frequency have not been investigated beyond the fusion of UGI and Mu Gam to diminish these outcomes (Komor et al., 2016b, 2017a; Nishida et al., 2016). Analyses of the comprehensive context library revealed that these rare outcomes varied somewhat by cell type. The purity of cytosine transversions resulting from CBE editing was elevated in mESCs compared to HEK293T and U2OS (mean of 2.8-16% of edited reads across CBEs in mESCs, compared to 2.6-9.5% in HEK293T and 1.6-7.7% in U2OS) and was accompanied by a slight increase in indels (1.3-fold and 2.1-fold relative to HEK293T and U2OS, respectively). This difference may be explained by elevated UNG in mESCs, which facilitates deoxyuracil excision to create an abasic site (Wu et al., 2013) that is an intermediate of transversion and indel formation.

Aside from cell type differences, cytosine product purities were also dependent on the CBE's cytidine deaminase. Targets with multiple editable cytosines were previously noted to yield C⋅G-to-T⋅A edits with greater purity than targets with only a single editable cytosine (Komor et al., 2017a), which predominantly relates to CBE window. Indeed, the base editor sequence-activity analysis confirmed that wide-window CBEs tended to have higher C⋅G-to-T⋅A product purities (Spearman r=−0.81, P=0.05, N=6 CBEs), yet, activity window size alone did not explain the variance in the frequency of rare outcomes among CBEs.

Additional factors that may affect CBE product purity were investigated, and it was found that rare outcomes of cytosine base editing appear non-uniform among fused deaminases. Transversion outcomes occurred at 4-fold higher frequency following eA3A-BE4 editing compared all other CBEs tested (approximately 12% compared to 3% average, respectively; FIGS. 1B-1I), and C⋅G-to-G⋅C outcomes were enriched relative to C⋅G-to-A⋅T conversion (˜3:1 for eA3A, compared to ˜3:2 mean for remaining CBEs). Editors that display the lowest frequency of cytosine transversion mutations include the narrow-window editor evoA-BE4 and the wide-window editor CDA-BE4 (FIGS. 1B-1I); however, these editors also displayed the lowest BE:indel ratios of their window classes (32:1 and 39:1, respectively). These findings strongly suggest that the deaminase components of CBEs not only create uracil products, but also play an additional, previously unrecognized role in the partitioning of outcomes that result from U⋅G mismatch repair.

### DISCUSSION

The abundance of base editors designed for the same basic task of either C⋅G-to-T⋅A mutation (CBEs) or A⋅T-to-G⋅C mutation (ABEs) complicates selection of the optimal tool for precision editing at a locus of interest. High-throughput base editing approaches to install disease-relevant SNVs or sgRNA-tiling of gene-regions holds promise for dissecting the functional role of sequences with fine granularity, and genome-wide perturbation by base editing has been shown to be less deleterious to cells than similar SpCas9-based screens (Hart et al., 2015; Koike-Yusa et al., 2013; Kuscu et al., 2017; Rajagopal et al., 2016; Shalem et al., 2014; Wang et al., 2014). High-throughput SpCas9-based screens often rely on sgRNA-input as a proxy for editing that occurs in the genome. The uncertainty regarding base editing outcomes, therefore, makes them less well-suited for such screens and high-throughput studies using base editors have remained limited (Kweon et al., 2019).

It was shown that base editing precision and efficiency are highly dependent on both editor and sequence context and frequently cannot be predicted from the target locus and known base editor characteristics by simple inspection. Comprehensive and systematic analysis of sequence and deaminase determinants of base editing outcomes has allowed us to build a suite of machine learning models to predict the genotypes resulting from base editing with high accuracy (R≈0.89), that can facilitate better design of base editing sgRNA targeting libraries to obtain expected genotypes. Predictable high-throughput base editing could further enable novel whole-genome assays to study disease-relevant sequence variations by massively parallel insertion of SNVs found in genome-wide association studies (GWAS) or to investigate the functional role of cancer point mutations (Bailey et al., 2018; Brown et al., 2019; Pardinas et al., 2018; Stahl et al., 2019).

Using the wealth of base editing data generated herein, the similarities and differences that define each editor were explained and insight was gained into the processes that take place in generating base editing outcomes. Apparent G⋅C-to-A⋅T editing was observed upstream of the sgRNA binding site, cytosine editing by ABEs, and identified sequence context as a driving force behind partitioning repair products of cytosine base editing which enabled us to identify cytosine transversion SNVs amenable to CBE-mediated correction by C⋅G-to-G⋅C and C⋅G-to-A⋅T conversion. Further, a new role for deaminase components of CBEs in affecting repair of deaminated cytosines was established. Collectively, these findings suggest a complex interaction of base editors, DNA repair proteins, and local sequence context that together determine the resulting edited product of base editing. It was demonstrated that the mutation of deaminase components of base editors can affect the relative frequency of cytosine base editing outcomes to either enrich or reduce transversions, suggesting that further engineering of base editors may uncover novel functionality to direct edits beyond the canonical by C⋅G-to-T⋅A editing by CBEs and A⋅T-to-G⋅C editing by ABEs with higher precision and efficiency.

Collectively, the extensive and minimally biased characterization of editing outcomes performed in this work provides both refined and novel insights into base editor functionality, advancing the scope, biological understanding, effectiveness, and precision of base editing.

### Star Methods

TABLE 1
Key Resources Table

| | | |
|---|---|---|
| REAGENT or RESOURCE | SOURCE | IDENTIFIER |

Bacterial and Virus Strains

| | | |
|---|---|---|
| NEB® 10-beta Competent E. coli | New England Biolabs | CAT#C3019H |

Chemicals, Peptides, and Recombinant Proteins

| | | |
|---|---|---|
| Lipofectamine 3000 | Thermo Fischer Scientific | CAT#L3000015 |
| Hygromycin B | Thermo Fischer Scientific | CAT#10687010 |
| Blasticidin | Thermo Fischer Scientific | CAT#A1113903 |
| Puromycin | Thermo Fischer Scientific | CAT#A1113803 |
| SspI-HF | New England Biolabs | CAT#R3132L |
| BbsI | New England Biolabs | CAT#R0539L |
| XbaI | New England Biolabs | CAT#R0145L |

Critical Commercial Assays

| | | |
|---|---|---|
| DNeasy Blood & Tissue Kit | QIAGEN | CAT#69504 |
| QIAquick PCR & Gel Cleanup Kit | QIAGEN | CAT#28506 |
| QIAquick PCR Purification Kit | QIAGEN | CAT#28104 |
| ZymoPURE™ II Plasmid Maxiprep Kit | Zymo Research | CAT#D4202 |
| NEBNext Ultra II Q5 Master Mix | New England Biolabs | CAT#M0544L |
| Gibson Assembly Master Mix | New England Biolabs | CAT#E2611L |
| Plasmid-Safe ATP-Dependent DNase | Lucigen | CAT#E3110K |
| TapeStation DNA ScreenTape & Reagents | Agilent | CAT#5067-5582, |
| | | 5067-5583 |
| KAPA Library Quantification Kit | KAPA Biosystems | CAT#KR0405 |
| NextSeq 500/550 High Output Kit | Illumina | CAT#20024907 |
| Miseq reagents kit v3 | Illumina | CAT#MS-102-3001 |

Deposited Data

| | | |
|---|---|---|
| Sequencing data | This study | PRJNA591007 |
| Processed editing efficiency data | This Example | doi.org/10.6084/ |
| | | m9.figshare.10673816 |
| Processed bystander editing data | This Example | doi.org/10.6084/ |
| | | m9.figshare.10678097 |

Experimental Models: Cell Lines

| | | |
|---|---|---|
| HEK293T | ATCC | CAT#-CRL-3216 |
| U2OS | ATCC | CAT#HTB-96 |
| P2L-mESC | Shen et al. 2018 | |

Oligonucleotides

| | | |
|---|---|---|
| Library cloning primer-Oligonucleotide | This Example | N/A |
| library Fw: | | |
| TTTTTGTTTTGTCTGTGTTCCGTTGTCCGTGCTG | | |
| TAACGAAAGgtgcagtNNNNNNNNNNNNNNN | | |
| GATGGGTGCGACGCGTCAT (SEQ ID NO: | | |
| 3257) | | |
| Library cloning primer-Oligonucleotide | This Example | N/A |
| library Rv: | | |
| GTTGATAACGGACTAGCCTTATTTAAACTTGCT | | |
| ATGCTGTTTCCAGCATAGCTCTTAAAC (SEQ ID | | |
| NO: 3258) | | |
| Library cloning primer-Circular donor Fw: | This Example | N/A |
| GTTTAAGAGCTATGCTGGAAACAGC (SEQ ID | | |
| NO: 3259) | | |
| Library cloning primer-Circular donor Rv: | This Example | N/A |
| ACTGCACCTTTCGTTACAGCACGGACAACGGA | | |
| ACACAGACAAAACAAAAAAGCACCGACTC | | |
| (SEQ ID NO: 3260) | | |
| Library cloning primer-Plasmid insert Fw: | This Example | N/A |
| TAACTTGAAAGTATTTCGATTTCTTGGCTTTAT | | |
| ATATCTTGTGGAAAGGACGAAACACCG (SEQ | | |
| ID NO: 3261) | | |
| Library cloning primer-Plasmid insert Rv: | This Example | N/A |
| TTGTGGTTTGTCCAAACTCATCAATGTATCTTA | | |
| TCATGTCTGCTCGAAGCGGCCGTACCTCTAGA | | |
| CACTCTTTCCCTACACGACGCTCTT (SEQ ID | | |
| NO: 3262) | | |
| Library sequencing primer-PCR1 Fw + | This Example | N/A |
| [Illumina BC]: | | |
| AATGATACGGCGACCACCGAGATCTACAC | | |
| [Illumina BC]ACACTCTTTCCCTACACGAC | | |
| (SEQ ID NO: 3263) | | |
| Library sequencing primer-PCR1 Rv: | This Example | N/A |
| GTGACTGGAGTTCAGACGTGTGCTCTTC | | |
| CGATCT GTGGAAAGGACGAAACACCG (SEQ | | |
| ID NO: 3264) | | |
| Library sequencing primer-PCR1 Fw: | This Example | N/A |
| AATGATACGGCGACCACCGAGATCTACAC | | |
| (SEQ ID NO: 3265) | | |
| Library sequencing primer-PCR2 Rv + | This Example | N/A |
| [Illumina BC]: | | |
| CAAGCAGAAGACGGCATACGAGAT[Illumina | | |
| BC]GTGACTGGAGTTCAGACGTGTGCTCTTC | | |
| (SEQ ID NO: 3266) | | |
| HEK2 sgRNA protospacer: | This Example | N/A |
| GAACACAAAGCATAGACTGC (SEQ ID NO: | | |
| 3267) | | |
| HEK3 sgRNA protospacer: | This Example | N/A |
| GAACACAAAGCATAGACTGC (SEQ ID NO: | | |
| 3267) | | |
| HEK4 sgRNA protospacer: | This Example | N/A |
| GGCACTGCGGCTGGAGGTGG (SEQ ID NO: | | |
| 3268) | | |
| b04 sgRNA protospacer: | This Example | N/A |
| GGCGTACTCCATGACAAAGC (SEQ ID NO: | | |
| 3269) | | |
| EMX sgRNA protospacer: | This Example | N/A |
| GAGTCCGAGCAGAAGAAGAA (SEQ ID NO: | | |
| 3270) | | |
| HEK2 Sequencing primer Fw: | This Example | N/A |
| CCAGCCCCATCTGTCAAACT (SEQ ID NO: | | |
| 3271) | | |
| HEK2 Sequencing primer Rv: | | |
| TGAATGGATTCCTTGGAAACAATGA (SEQ ID | | |
| NO: 3272) | | |
| HEK3 Sequencing primer Fw: | | |
| ATGTGGGCTGCCTAGAAAGG (SEQ ID NO: | | |
| 3273) | | |
| HEK3 Sequencing primer Rv: | | |
| CCCAGCCAAACTTGTCAACC (SEQ ID NO: | | |
| 3274) | | |
| HEK4 Sequencing primer Fw: | | |
| GAACCCAGGTAGCCAGAGAC (SEQ ID NO: | | |
| 3275) | | |
| HEK4 Sequencing primer Rv: | | |
| TCCTTTCAACCCGAACGGAG (SEQ ID NO: | | |
| 3276) | | |
| b04 Sequencing primer Fw: | | |
| GTCTGGTGCCATGGAGAGTAG (SEQ ID NO: | | |
| 3277) | | |
| b04 Sequencing primer Rv: | | |
| GGTATCAGGCGACGTGGTAT (SEQ ID NO: | | |
| 3278) | | |
| EMX Sequencing primer Rv: | | |
| CAGCTCAGCCTGAGTGTTGA (SEQ ID NO: | | |
| 3279) | | |
| EMX Sequencing primer Rv: | | |
| CTCGTGGGTTTGTGGTTGC (SEQ ID NO: 3280) | | |

Recombinant DNA

| | | |
|---|---|---|
| Tol2 trasposase | Shen et al. 2018 | Tol2 |
| p2Tol-U6-2xBbsI-sgRNA-HygR | Arbab et al. 2018 | Addgene #71485 |
| p2T-CAG-SpCas9-BlastR | Arbab et al. 2018 | Addgene #107190 |
| p2T-CMV-ABEmax-BlastR | This Example | ABE |
| p2T-CMV-ABEmax-CP1041-BlastR | This Example | ABE-CP |
| p2T-CMV-BE4max-BlastR | This Example | BE4 |
| p2T-CMV-BE4max-CP1028-BlastR | This Example | BE4-CP |
| p2T-CMV-AIDmax-BlastR | This Example | AID-BE4 |
| p2T-CMV-CDAmax-BlastR | This Example | CDA-BE4 |
| p2T-CMV-evoAPOBEC1max-BlastR | This Example | evoA-BE4 |
| p2T-CMV-eA3Amax-BlastR | This Example | eA3A-BE4 |
| p2T-CMV-eA3Amax-NG-BlastR | This Example | eA3A-BE4-NG |
| p2T-CMV-eA3Amax-T31A-NG-BlastR | This Example | eA3A-NG(T31A) |
| p2T-CMV-BE4max-H47E + S48A-BlastR | This Example | EA-BE4 |
| p2T-CMV-eA3Amax-T44D + S45A-BlastR | This Example | eA3A-BE5 |

Software and Algorithms

| | | |
|---|---|---|
| Code repository for data processing | This Example | github.com/ |
| | | maxwshen/lib- |
| | | dataprocessing |
| Code repository for data analysis | This Example | github.com/maxwshen/ |
| | | lib-analysis |
| Code repository for the editing | This Example | github.com/maxwshen/ |
| efficiency model | | be_predict_efficiency |
| Code repository for the bystander | This Example | github.com/maxwshen/ |
| editing model | | be_predict_bystander |
| Theseus | Theobald et al. 2012 | |

### Methods

### Library Cloning

The cloning process is as reported in Shen et al. 2018, with minor changes. In brief, the process involves ordering a library of 2,000 to 12,000 oligonucleotides pairing an sgRNA protospacer with its 35-nt, 56-nt or 61-nt target site, centered on an NGG or NG PAM, as specified. Pools were amplified with NEBNext Ultra II Q5 Master Mix (New England Biolabs) with initial denaturation and extension times extended to 2 minutes per cycle for all PCR reactions to prevent skewing towards GC-rich sequences. To insert the sgRNA hairpin between the sgRNA protospacer and the target site, the library undergoes an intermediate Gibson Assembly circularization step, restriction enzyme linearization and Gibson Assembly into a plasmid backbone containing a U6 promoter to facilitate sgRNA expression, a hygromycin resistance cassette and flanking Tol2 transposon sites to facilitate integration into the genome. Purified plasmids were transformed into NEB10beta (New England Biolabs) electrocompetent cells. Following recovery, a small dilution series was plated to assess transformation efficiency and the remainder was grown in liquid culture in DRM medium overnight at 37° C. with 100 ug/mL ampicillin. The plasmid library was isolated by Midiprep plasmid purification (Qiagen). Library integrity was verified by restriction digest with SapI (New England Biolabs) for 1 hour at 37° C., and sequence diversity was validated by deep sequencing as described below.

### Cloning

Base editor plasmids were constructed by inserting a blasticidin resistance expression cassette from a p2T-CAG-SpCas9-BlastR plasmid (107190) (Arbab et al., 2015) downstream of the bGH-polyA terminator into a BE4 plasmid (100802) (Komor et al., 2017). Tol2-transposase sites from p2T-CAG-SpCas9-BlastR were cloned to flank the base editor and antibiotic selection cassettes. All editors described in this Example were cloned between the N-terminal and C-terminal NLS sequences flanking BE4. The full sequence of the p2T-CAG-BE4max-BlastR plasmid and editor sequences for all editors used in this Example is appended in the ‘Sequences’ section.

Individual SpCas9 sgRNAs were cloned as a pool into a Tol2-transposon-containing gRNA expression plasmid (Addgene 71485) using BbsI plasmid digest and Gibson Assembly (New England Biolabs). Protospacer sequences and gene specific primers used for amplification followed by HTS are listed in the Primers Table.

### Cell Culture

mESC lines used have been described previously and were cultured as described previously (Sherwood et al., 2014). HEK293T and U20S cells were purchased from ATCC and cultured as recommended by ATCC. Cell lines were authenticated by the suppliers and tested negative for Mycoplasma.

For stable Tol2 transposon library integration, cells were transfected using Lipofectamine 3000 (Thermo Fisher) following standard protocols with equimolar amounts of Tol2 transposase plasmid (a gift from K. Kawakami) and transposon-containing plasmid. For library applications, 15-cm plates with >10⁷ initial cells were used, and for single sgRNA targeting, 48-well plates with >10⁵ initial cells were used. To generate library cell lines with stable Tol2-mediated genomic integration, cells were selected with hygromycin starting the day after transfection at an empirically defined concentration and continued for >2 weeks. In cases where sequential plasmid integration was performed such as integrating library and then base editor, cells were transfected with Tol2 transposase plasmid using Lipofectamine 3000 and selected with blasticidin starting the day after transfection for 4 days before harvesting.

### Deep Sequencing

Genomic DNA was collected from cells 5 days after transfection, after 4 days of antibiotic selection. For library samples, 16 μg gDNA was used for each sample; for individual locus samples and untreated cell library samples, 2 μg gDNA was used; for plasmid library verification, 0.5 μg purified plasmid DNA was used. For individual locus samples, the locus surrounding CRISPR-Cas9 mutation was PCR-amplified in two steps using primers >50-bp from the Cas9 target site. PCR1 was performed to amplify the endogenous locus or library cassette using the primers specified below. PCR2 was performed to add full-length Illumina sequencing adapters using the NEBNext Index Primer Sets 1 and 2 (New England Biolabs) or internally ordered primers with equivalent sequences. All PCRs were performed using NEBNext Ultra II Q5 Master Mix. Extension time for all PCR reactions was extended to 2 minutes per cycle to prevent skewing towards GC-rich sequences. Samples were pooled using Tape Station (Agilent) and quantified using a KAPA Library Quantification Kit (KAPA Biosystems). The pooled samples were sequenced using NextSeq or MiSeq (Illumina).

### Library Names

Supplementary figures, tables, and deposited data use different names for designed libraries than the manuscript for convenience. The “comprehensive context library” is referred to as “12kChar” and contains 12,000 target sites designed with all 4-mers surrounding a substrate nucleotide at protospacer positions 1-11 and all 6-mers surrounding an adenine or cytosine at position 6. Three disease-associated libraries called “CBE precision editing SNV library”, “ABE precision editing SNV library”, and “transversion-enriched SNV library” in the manuscript are referred to as “CtoT”, “AtoG”, and “CtoGA”, indicating the base editing event that corrects the disease-related variants included in each library.

### Sequence Motif Models

For prediction tasks where the target variable is continuous and has range in (0, 1), a logistic transformation to the data was applied, and then linear regression was used. For continuous data representing fractions, values equal to 0 or 1 were discarded. For classification tasks, the target variables were either 0 or 1 indicating absence or presence of activity, and logistic regression was used. Target variables included the efficiency of C⋅G-to-T⋅A editing by CBEs, A⋅T-to-G⋅C editing by ABEs, the presence or absence of cytosine editing by ABEs and of guanine editing by CBEs, and the purity of cytosine transversions by CBEs. Each of these statistics involves calculating a denominator corresponding to the total number of reads at a target sequence, or the total number of edited reads at a target sequence. Target sequences with fewer than 100 reads in the denominator were discarded to ensure the accuracy of estimated statistics in the training and testing data. Features were obtained by one-hot-encoding nucleotides per position relative to a substrate nucleotide or to the protospacer. The data were randomly split into training and test sets at an 80:20 ratio. Sequence motifs described by these regression models consider each position independently and are intended primarily for visualization.

### Base Editing Efficiency Model

Base editing efficiency varies by experimental batch. To combine replicates across batches, first a mean centering and logit transformation was performed at up to 10,638 gRNA-target pairs in each experimental condition separately from the 12kChar library which includes all 4-mers surrounding A or C from protospacer positions 1 to 11. Data was discarded at target sites with fewer than 100 total reads, then averaged values at matched target sites across experimental replicates. Values of negative or positive infinity (resulting from logit of 0 or 1) were discarded. The data were randomly split into training and test sets at a ratio of 90:10. Each target site had a single output value corresponding to the mean logit fraction of sequenced reads with any base editing activity. Data points comprising a single replicate were assigned weight=0.5. Data points comprising multiple replicates were assigned a weight of the median logit variance divided by the logit variance at that data point, or 1, whichever value was smaller. In this manner, exactly half of the data points comprising multiple replicates were assigned a weight of 1, and those with higher variance were assigned a lower weight. Features from each target sequence were obtained using protospacer positions −9 to 21. Features included one-hot encoded single nucleotide identities at each position, one-hot encoded dinucleotides at neighboring positions, the melting temperature of the sequence and various subsequences, the total number of each nucleotide in the sequence, and the total number of G or C nucleotides in the sequence. Gradient-boosted regression trees from the python package scikit-learn were used, and trained with tuples of (x, y, weights) using the training data. Hyperparameter optimization was performed by varying the number of estimators between {100, 250, 500}, the minimum samples per leaf in {2, 5}, and the maximum tree depth in {2, 3, 4, 5}. A 5-fold cross-validation was performed by splitting the training set into a training and validation set at a ratio of 8:1 and retained the combination of hyperparameters with the strongest average cross-validation performance as the final model. Models were trained in this manner for each combination of cell-type and base editor. Models were evaluated on the test set which was not used during hyperparameter optimization.

### Bystander Editing Model

A dataset was assembled where each gRNA-target pair was matched with a table of observed base editing genotypes and their frequencies among reads with edited outcomes. Data points with fewer than 100 edited reads were discarded. Edited genotypes occurring at higher than 2.5% frequency with no edits at any substrate nucleotides (defined as C for CBEs and A for ABEs) in positions 1-10 were also discarded. Data from multiple experimental replicates were combined by summing read counts for each observed genotype.

Briefly, a deep conditional autoregressive model was designed and implemented that used an input target sequence surrounding a protospacer and PAM to output a frequency distribution on combinations of base editing outcomes in the python package pytorch. The model predicts substitutions at cytosines and guanines for CBEs and adenines and cytosines for ABEs from protospacer positions −10 to 20. The model transforms each substrate nucleotide and its local context using a shared encoder into a deep representation, then applies an autoregressive decoder that iteratively generates a distribution over base editing outcomes at each substrate nucleotide while conditioning on all previous generated outcomes. The encoder and decoder are coupled with a learned position-wise bias towards producing an unedited outcome. The model is trained on observed data by minimizing the KL divergence. Importantly, the conditional autoregressive design is sufficiently expressive to learn any possible joint distribution in the output space, thereby representing a powerful and general method for learning the editing tendencies of any base editor from data.

Input features were obtained by one-hot encoding each substrate nucleotide and the 5 nucleotides (where 5 is a hyperparameter) on either side of it and concatenating this with a one-hot encoding of the position of the substrate nucleotide within positions −9 to 20. Additional features considered but found to detract from model performance during hyperparameter optimization included concatenating a one-hot encoding of the full sequence context. Hyperparameter optimization on the radii of nucleotides surrounding the substrate nucleotide considered values in {3, 5, 7, 9}, and found 5 to be optimal when averaged across hyperparameter optimization rounds that included simultaneous changes in other hyperparameters. Each substrate nucleotide within the editing range were featurized in this manner for each target sequence.

The model uses two neural networks: an encoder with two hidden layers of 64 neurons and a decoder with five hidden layers of 64 neurons. The networks are fully connected, use ReLU activations, and contain residual connections between neighboring pairs of layers that have equal shape. A dropout frequency of 5.0% was used and tuned by hyperparameter optimization. An architecture search in hyperparameter optimization was included and found that these shapes were a local optimum in the surrounding neighborhood varying the number of neurons per layer and the number of layers in each network.

During a forward pass of the model at a single target site, the shapes of relevant variables are:
x.shape=(n.edit.b, x_dim)
y_mask.shape=(n.uniq.e+1, n.edit.b, y_mask_dim)
target.shape=(n.uniq.e+1, n.edit.b, 4, 1)
obs_freq.shape=(n.uniq.e)
   where:
‘x’ is the featurized input
‘y_mask’ is used to provide previously observed outcomes to the decoder while masking future outcomes, in a conditional autoregressive manner
‘target’ is a one-hot encoding of each unique edited genotype
‘obs_freq’ contains the observed frequencies for each edited genotype
n.uniq.e=the number of unique observed edited genotypes for a target site
n.edit.b=the number of editable bases in the target sequence
x_dim=the number of features for a single substrate nucleotide in a single target sequence

The shape n.uniq.e+1 is used to indicate the inclusion of a row for the wild-type outcome. The model was run on this outcome and the result was used to adjust all predicted probabilities to obtain a denominator equal to 1−p(wild-type).

The tensor ‘y_mask’ was used to provide previously observed outcomes to the decoder while masking future outcomes in a conditional autoregressive fashion. Previously observed unedited nucleotides are encoded as [1/3, 1/3, 1/3], while editable nucleotides are encoded as [0, 0, 0] if unedited, and otherwise are a one-hot encoding of the nucleotide resulting from the base edit. Future nucleotides are encoded as [−1, −1, −1].

The following shape transformations occur during a forward pass.
1. Model encodes x: (n.edit.b, x_dim)→(n.edit.b, x_enc_dim)
2. Expanding and concatenating with y_mask→(n.uniq.e+1, n.edit.b, x_enc_dim+y_mask_dim).
3. Decode→(n.uniq.e+1, n.edit.b, 1, 4)
4. Add unedited bias, then log softmax→(n.uniq.e+1, n.edit.b, 1, 4)
5. Matrix multiplication with target one-hot-encoding→(n.uniq.e+1, n.edit.b, 1, 1), reshape→(n.uniq.e+1, n.edit.b)
6. Sum log likelihoods→(n.uniq.e+1)
7. Adjust all likelihoods by (1−wild-type) denominator→(n.uniq.e). The wild-type outcome is encoded at the last position.

The resulting (n.uniq.e) shape vector contains a number corresponding to the predicted frequency of each unique observed genotype (totaling n.uniq.e). To obtain a loss during training, the KL divergence between the predicted frequency distribution and the observed frequency distribution is used.

A learnable bias toward unedited outcomes is a part of the model. This component uses an input shape of (n.uniq.e+1, n.edit.b, 1, 4) and outputs a tensor with equivalent shape: (n.uniq.e+1, n.edit.b, 1, 4). Its parameters correspond to a single value for each position and substrate nucleotide representing a bias towards producing an unedited outcome. One important aspect of the structure of the data is that most dimensions of the input and output tensors vary by target site. Batches comprised of groups of target sites. Empirically, it was observed that this property caused minimal speed gains when training the model on CPUs vs GPUs.

### Quantification and Statistical Analysis

### Sequence Alignment and Data Processing

Sequencing reads were assigned to designed library target sites by locality sensitive hashing). Target contexts that were intentionally designed to be highly similar to each other were designed barcodes to assist accurate assignment. Sequence alignment was performed using Smith-Waterman with the parameters: match +1, mismatch −1, indel start −5, indel extend 0. Nucleotides with PHRED score below 30 were assumed to be the reference nucleotide.

For base editing analysis, aligned reads with no indels were retained for analysis and events were defined as the combination of all possible substitutions at all substrate nucleotides in the target site in a read, where a single sequencing read corresponds to an observation of a single event. Substrate nucleotides were defined as C and G for CBEs and A and C for ABEs.
For indel analysis, reads containing indels with at least one indel position occurring between protospacer positions −6 to 26 were retained, where position 1 is the 5′-most nucleotide of the protospacer, and 0 is used to refer to the position between −1 and 1. Reads containing indels without at least six nucleotides with at least 90% match frequency on both sides of each indel were discarded. Events were defined as indels identified by position, length, and inserted nucleotides occurring in a read. Combination indels were either not observed at all or only at exceedingly low frequencies in endogenous data and were therefore excluded from consideration when analyzing library data.

### Quantifying Base Editing Profiles

The frequencies of each single-nucleotide mutation were tabulated at each position in each designed target sequence from the sequence alignments. Then, the following steps were applied to adjust treatment data by control data, adjust batch effects and identify base editing mutations that occur at frequencies above background.

The first step was to filter control mutations in control data occurring at or above a 5.0% frequency threshold. As control conditions do not undergo a second selection step (90-95% cell death then expansion), control mutations that are relatively common are highly likely to expand in frequency in treatment data. Since the resulting treatment population frequency (before editing) has high variance (due to the 90-95% cell death then expansion), it is very difficult to de-confound this factor from mutations occurring due to base editing.

The second step was to filter treatment mutations that could be explained by control mutations. The probability of treatment mutations occurring from a binomial distribution parameterized by the observed mutation frequency in the control population and filter mutations was determined at FDR=0.05.

The third step was to filter mutations occurring in both control and treatment conditions, subtract control frequencies from treatment frequencies.

The fourth step was to filter treatment mutations that could be explained by Illumina sequencing errors. The probability of treatment mutations was determined under a binomial distribution parameterized by the lowest quality (>Q30) sequencing call at that position and filter at FDR=0.05. The empirical determined lowest quality is often Q32 or Q36, which correspond to error thresholds of 6e-4 and 2e-4 respectively.

The fifth step was to filter treatment mutations that could be explained by batch effects (comparing treatment vs. treatment). Summary statistics of the mean mutation rate were calculated across all target site with a given substrate nucleotide at a particular position to another nucleotide, yielding an L×12 matrix for each condition, where L=55, 56, or 61. Then, perform one-way ANOVA was performed using the batches defined on the first slide and filter mutations at Bonferroni-corrected p-value threshold of 0.005.

The sixth step was to identify treatment mutations that were consistent by editors across conditions, especially rare ones, while filtering background mutations (comparing treatment vs. treatment). On the batch-effect-corrected L×12 matrix per condition, group by editors, calculate normalized rankings of each mutation within each condition. Perform robust rank aggregation on each mutation to obtain an upper bound on the p-value.

Based on the above analysis, editing profiles were empirically designed for denoising and filtering base editing outcomes. To ensure high sensitivity, these profiles were designed to be broad to minimize the possibility of excluding reads with legitimate base editing activity. For CBEs, base editing activity was defined as C to A, G, or T at positions −9 to 20 and G to A or C at positions −9 to 5. For ABEs, base editing activity was defined as A to G at positions −5 to 20, A to C or T at positions 1 to 10, and C to G or T at positions 1 to 10. For all analysis described herein that required tabulating reads with base editing activity, reads were discarded that did not have base editing activity according to these broad profiles.

Selection of Variants from Disease Databases

Disease variants were selected from the NCBI ClinVar database and the Human Gene Mutation Database (HGMD) for computational screening and subsequent experimental correction using versions of both database that were up to date as of September of 2018. Variants from ClinVar that were designated by at least one lab as ‘pathogenic’ or ‘likely pathogenic’ were retained. Variants from HGMD with a disease association of ‘DM’ or disease-causing mutation were retained.

SpCas9 gRNAs were enumerated for each disease allele. Using a previous version of BE-Hive, predicted correction precisions were predicted for each gRNA-allele combination and used to prioritize the design of libraries. Two libraries of 12,000 gRNA-target pairs were designed called ‘AtoG’ and ‘CtoT’. The ‘AtoG’ library contained 11,585 unique pathogenic variants while ‘CtoT’ contained 7,444 unique pathogenic variants. A third library ‘CtoGA’ with 3,800 gRNA-target pairs targeting pathogenic variants was designed with 2,668 unique pathogenic variants.

### Quantifying the Ratio of Base Editing to Indel Activity

Target sites with greater than 1000 reads and with at least one indel read were retained (to avoid division by zero). Notably, no pseudocounts were used. To calculate BE:indel ratios, library target sites without a substrate nucleotide within the typical base editing window were filtered. These target sites resulted from the library design choices that prioritized diversity and exploration, but these target sites are unlikely to be selected for editing in common user applications. The geometric mean was selected as a summary statistic because BE:indel ratios were distributed roughly log-normal, and the statistic summarizes more of the data than the median.

### Adjusting for Noise in 1-bp Indels

To characterize rare indels from base editing outcomes, endogenous data (with large sequencing depth, in HEK293T cells) was used and designed certain library conditions were designed (with high editing efficiency and deep sequencing coverage) as gold standards to denoise the other library datasets. In both endogenous data and gold-standard library conditions, the fraction of 1-bp indels was observed to be 5-30% of all indels. In contrast, in many treatment library conditions, the fraction was as high as 80-95%, similar to those in untreated library controls. In addition, these background 1-bp indels appeared to occur nearly uniformly across the target site, while in the “gold standard” conditions, 1-bp indels are concentrated near the HNH nick and typical base editing window. Based on these sets of observations, it was reasoned that the conservative adjustment of treatment conditions by control conditions (by subtracting the frequency of indels at matching target sites, with matching indel start position and length) did not completely adjust noise from treatment data. To enable a more accurate calculation of base editing to indel ratios, an additional quality control step was applied where the frequencies of 1-bp indels in library target sites were decreased uniformly such that the global (across the entire library of sequence contexts) frequency of 1-bp indels was at most 30% of all indels.

### Adjusting for Batch Effects in Base Editing to Indel Ratios

Some batch effects in calculated BE:indel ratios were observed. To adjust for batch effects, two-way ANOVA was applied, crossing experimental batch with base editor, on the geometric mean BE:indel ratio for all library experiments. As instructed by the experimental protocol, the batch must be distinct for each combination of cell-type and library. For this analysis, all point mutants of base editors were dinned with their wild-type versions since small differences in BE:indel ratios were observed that were dominated by differences by experimental batch and by base editor. The average coefficient across all experimental batches was added to the learned coefficient for each base editor to obtain a batch-adjusted coefficient for each base editor. An adjustment factor was obtained as the difference between the average geometric mean BE:indel ratio across experiments for a given base editor and the batch-adjusted coefficient for that base editor. Adjustment factors were used to adjust the BE:indel ratio at individual target sites for analysis requiring such resolution.

### Definition of Disequilibrium Score

Disequilibrium scores are calculated for a given pair of substrate nucleotides as the ratio between the observed joint editing probability and the probability of both nucleotides being edited together assuming statistical independence. Calculating a valid log disequilibrium score from observed data requires non-zero frequencies for p(first nucleotide is edited), p(second nucleotide is edited), and p(first and second nucleotide are edited). Disequilibrium score values above one indicate a tendency for both or neither to be edited together (positive log disequilibrium score), while values below one indicate a tendency for only one or the other to be edited (negative log disequilibrium score).

### Data and Code Availability

The sequencing data generated herein are available at the NCBI Sequence Read Archive database under PRJNA591007. Processed data have been deposited under the following DOIs: 10.6084/m9.figshare.10673816 and 10.6084/m9.figshare.10678097. The code used for data processing and analysis are available at github.com/maxwshen/lib-dataprocessing and github.com/maxwshen/lib-analysis.

### Additional Resources

Interactive web application for BE-Hive can be found at crisprbehive.design. The Python package for BE-Hive can be found at github.com/maxwshen/be_predict_efficiency and github.com/maxwshen/be_predict_bystander.

### REFERENCES FOR EXAMPLE 1

1. Adli, M. (2018). The CRISPR tool kit for genome editing and beyond. Nat. Commun. 9, 1911.
2. Adolph, M. B., Love, R. P., Feng, Y., and Chelico, L. (2017). Enzyme cycling contributes to efficient induction of genome mutagenesis by the cytidine deaminase APOBEC3B. Nucleic Acids Res. 45, 11925-11940.
3. Anzalone, A. V., Randolph, P. B., Davis, J. R., Sousa, A. A., Koblan, L. W., Levy, J. M., Chen, P. J., Wilson, C., Newby, G. A., Raguram, A., et al. (2019). Search-and-replace genome editing without double-strand breaks or donor DNA. Nature.
4. Arbab, M., Srinivasan, S., Hashimoto, T., Geijsen, N., and Sherwood, R. I. (2015). Cloning-free CRISPR. Stem Cell Rep. 5, 1-10.
5. Bailey, M. H., Tokheim, C., Porta-Pardo, E., Sengupta, S., Bertrand, D., Weerasinghe, A., Colaprico, A., Wendl, M. C., Kim, J., Reardon, B., et al. (2018). Comprehensive Characterization of Cancer Driver Genes and Mutations. Cell 173, 371-385.e18.
6. Barkal, A. A., Srinivasan, S., Hashimoto, T., Gifford, D. K., and Sherwood, R. I. (2016). Cas9 Functionally Opens Chromatin.
7. Basu, U., Chaudhuri, J., Alpert, C., Dutt, S., Ranganath, S., Li, G., Schrum, J. P., Manis, J. P., and Alt, F. W. (2005). The AID antibody diversification enzyme is regulated by protein kinase a phosphorylation. Nature 438, 508-511.
8. Beale, R. C. L., Petersen-Mahrt, S. K., Watt, I. N., Harris, R. S., Rada, C., and Neuberger, M. S. (2004). Comparison of the Differential Context-dependence of DNA Deamination by APOBEC Enzymes: Correlation with Mutation Spectra in Vivo. J. Mol. Biol. 337, 585-596.
9. Blom, N., Sicheritz-Pontén, T., Gupta, R., Gammeltoft, S., and Brunak, S. (2004). Prediction of post-translational glycosylation and phosphorylation of proteins from the amino acid sequence. PROTEOMICS 4, 1633-1649.
10. Brown, A.-L., Li, M., Goncearenco, A., and Panchenko, A. R. (2019). Finding driver mutations in cancer: Elucidating the role of background mutational processes. PLOS Comput. Biol. 15, e1006981.
11. Chadwick, A. C., Wang, X., and Musunuru, K. (2017). In Vivo Base Editing of PCSK9 (Proprotein Convertase Subtilisin/Kexin Type 9) as a Therapeutic Alternative to Genome Editing. Arter. Thromb Vasc Biol 37, 1741-1747.
12. Chaudhuri, A., and Behan, P. O. (2004). Fatigue in neurological disorders. The Lancet 363, 978-988.
13. Chen, B., Gilbert, L. A., Cimini, B. A., Schnitzbauer, J., Zhang, W., Li, G.-W., Park, J., Blackburn, E. H., Weissman, J. S., Qi, L. S., et al. (2013). Dynamic Imaging of Genomic Loci in Living Human Cells by an Optimized CRISPR/Cas System. Cell 155, 1479-1491.
14. Cong, L., Ran, F. A., Cox, D., Lin, S., Barretto, R., Habib, N., Hsu, P. D., Wu, X., Jiang, W., Marraffini, L. A., et al. (2013). Multiplex Genome Engineering Using CRISPR/Cas Systems. Science 339, 819.
15. Doench, J. G., Fusi, N., Sullender, M., Hegde, M., Vaimberg, E. W., Donovan, K. F., Smith, I., Tothova, Z., Wilen, C., Orchard, R., et al. (2016). Optimized sgRNA design to maximize activity and minimize off-target effects of CRISPR-Cas9. Nat. Biotechnol. 34, 184-191.
16. Doshi, B. S., and Arruda, V. R. (2018). Gene therapy for hemophilia: what does the future hold? Ther. Adv. Hematol. 9, 273-293.
17. Doudna, J., and Knott, G. (2018). CRISPR-Cas guides the future of genetic engineering. Science 361, 1-4.
18. Friedman, J. H. (2001). GreedyFunctionApproximation.GradientBoostingMachine.Friedman (1999). Ann. Stat. 29, 1189-1232.
19. Gaudelli, N. M., Komor, A. C., Rees, H. A., Packer, M. S., Badran, A. H., Bryson, D. I., and Liu, D. R. (2017). Programmable base editing of A⋅T to G⋅C in genomic DNA without DNA cleavage. Nature 551, 464-471.
20. Gehrke, J. M., Cervantes, O., Clement, M. K., Wu, Y., Zeng, J., Bauer, D. E., Pinello, L., and Joung, J. K. (2018). An APOBeC3A-Cas9 base editor with minimized bystander and off-target activities. Nat. Biotechnol. 36, 977.
21. Hart, T., Chandrashekhar, M., Aregger, M., Steinhart, Z., Brown, K. R., MacLeod, G., Mis, M., Zimmermann, M., Fradet-Turcotte, A., Sun, S., et al. (2015). High-Resolution CRISPR Screens Reveal Fitness Genes and Genotype-Specific Cancer Liabilities. Cell 163, 1515-1526.
22. Huang, T. P., Zhao, K. T., Miller, S. M., Gaudelli, N. M., Oakes, B. L., Fellmann, C., Savage, D. F., and Liu, D. R. (2019). Circularly permuted and PAM-modified Cas9 variants broaden the targeting scope of base editors. Nat. Biotechnol.
23. Imai, K., Slupphaug, G., Lee, W.-I., Revy, P., Nonoyama, S., Catalan, N., Yel, L., Forveille, M., Kavli, B., Krokan, H. E., et al. (2003). Human uracil-DNA glycosylase deficiency associated with profoundly impaired immunoglobulin class-switch recombination. Nat. Immunol. 4, 1023-1028.
24. Jinek, M., East, A., Cheng, A., Lin, S., Ma, E., and Doudna, J. (2013). RNA-programmed genome editing in human cells. ELife 2, e00471.
25. Kalia, S. S., Adelman, K., Bale, S. J., Chung, W. K., Eng, C., Evans, J. P., Herman, G. E., Hufnagel, S. B., Klein, T. E., Korf, B. R., et al. (2016). Recommendations for reporting of secondary findings in clinical exome and genome sequencing, 2016 update (ACMG SF v2.0): a policy statement of the American College of Medical Genetics and Genomics. Genet. Med. 19, 249.
26. Kim, H. S., Jeong, Y. K., Hur, J. K., Kim, J.-S., and Bae, S. (2019). Adenine base editors catalyze cytosine conversions in human cells. Nat. Biotechnol.
27. Koblan, L. W., Doman, J. L., Wilson, C., Levy, J. M., Tay, T., Newby, G. A., Maianti, J. P., Raguram, A., and Liu, D. R. (2018). Improving cytidine and adenine base editors by expression optimization and ancestral reconstruction. Nat. Biotechnol. 36, 843-848.
28. Koike-Yusa, H., Li, Y., Tan, E.-P., Velasco-Herrera, M. D. C., and Yusa, K. (2013). Genome-wide recessive genetic screening in mammalian cells with a lentiviral CRISPR-guide RNA library. Nat. Biotechnol. 32, 267-273.
29. Komor, A. C., Kim, Y. B., Packer, M. S., Zuris, J. A., and Liu, D. R. (2016). Programmable editing of a target base in genomic DNA without double-stranded DNA cleavage. Nature 533, 420-424.
30. Komor, A. C., Zhao, K. T., Packer, M. S., Gaudelli, N. M., Waterbury, A. L., Koblan, L. W., Kim, Y. B., Badran, A. H., and Liu, D. R. (2017). Improved base excision repair inhibition and bacteriophage Mu Gam protein yields C:G-to-T:A base editors with higher efficiency and product purity. Sci. Adv. 3, eaao4774.
31. Kuscu, C., Parlak, M., Tufan, T., Yang, J., Szlachta, K., Wei, X., Mammadov, R., and Adli, M. (2017). CRISPR-STOP: gene silencing through base-editing-induced nonsense mutations. Nat. Methods 14, 710-712.
32. Kweon, J., Jang, A.-H., Shin, H. R., See, J.-E., Lee, W., Lee, J. W., Chang, S., Kim, K., and Kim, Y. (2019). A CRISPR-based base-editing screen for the functional assessment of BRCA1 variants. Oncogene.
33. Landrum, M. J., Lee, J. M., Benson, M., Brown, G., Chao, C., Chitipiralla, S., Gu, B., Hart, J., Hoffman, D., Hoover, J., et al. (2016). ClinVar: public archive of interpretations of clinically relevant variants. Nucleic Acids Res 44, D862-8.
34. Liang, P., Ding, C., Sun, H., Xie, X., Xu, Y., Zhang, X., Sun, Y., Xiong, Y., Ma, W., Liu, Y., et al. (2017). Correction of 0-thalassemia mutant by base editor in human embryos. Protein Cell 8, 811-822.
35. Lin, S., Staahl, B. T., Alla, R. K., and Doudna, J. A. (2014). Enhanced homology-directed human genome engineering by controlled timing of CRISPR/Cas9 delivery. ELife 3, e04766-e04766.
36. Liu, L. D., Huang, M., Dai, P., Liu, T., Fan, S., Cheng, X., Zhao, Y., Yeap, L. S., and Meng, F. L. (2018). Intrinsic Nucleotide Preference of Diversifying Base Editors Guides Antibody Ex Vivo Affinity Maturation. Cell Rep. 25, 884-892.e3.
37. Love, R. P., Xu, H., and Chelico, L. (2012). Biochemical analysis of hypermutation by the deoxycytidine deaminase APOBEC3A. J. Biol. Chem. 287, 30812-30822.
38. Ma, H., Wu, Y., Dang, Y., Choi, J.-G., Zhang, J., and Wu, H. (2014). Pol III Promoters to Express Small RNAs: Delineation of Transcription Initiation. Mol. Ther. Nucleic Acids 3, e161.
39. Mali, P., Yang, L., Esvelt, K. M., Aach, J., Guell, M., DiCarlo, J. E., Norville, J. E., and Church, G. M. (2013). RNA-Guided Human Genome Engineering via Cas9. Science 339, 823.
40. McBride, K. M., Gazumyan, A., Woo, E. M., Schwickert, T. A., Chait, B. T., and Nussenzweig, M. C. (2008). Regulation of class switch recombination and somatic mutation by AID phosphorylation. J. Exp. Med. 205, 2585-2594.
41. Min, Y.-L., Bassel-Duby, R., and Olson, E. N. (2019). CRISPR Correction of Duchenne Muscular Dystrophy. Annu. Rev. Med. 70, 239-255.
42. Molla, K. A., and Yang, Y. (2019). CRISPR/Cas-Mediated Base Editing: Technical Considerations and Practical Applications. Trends Biotechnol. 37, 1121-1142.
43. Nishida, K., Arazoe, T., Yachie, N., Banno, S., Kakimoto, M., Tabata, M., Mochizuki, M., Miyabe, A., Araki, M., Hara, K. Y., et al. (2016). Targeted nucleotide editing using hybrid prokaryotic and vertebrate adaptive immune systems. Science 353, aaf8729.
44. Nishimasu, H., Shi, X., Ishiguro, S., Gao, L., Hirano, S., Okazaki, S., Noda, T., Abudayyeh, O. O., Gootenberg, J. S., Mori, H., et al. (2018). Engineered CRISPR-Cas9 nuclease with expanded targeting space. Science 361, 1259.
45. Van Den Oord, A., Kalchbrenner, N., and Kavukcuoglu, K. (2016). Pixel recurrent neural networks. In 33rd International Conference on Machine Learning, ICML 2016, pp. 2611-2620.
46. Paquet, D., Kwart, D., Chen, A., Sproul, A., Jacob, S., Teo, S., Olsen, K. M., Gregg, A., Noggle, S., and Tessier-Lavigne, M. (2016). Efficient introduction of specific homozygous and heterozygous mutations using CRISPR/Cas9. Nature 533, 1-18.
47. Pardinas, A. F., Holmans, P., Pocklington, A. J., Escott-Price, V., Ripke, S., Carrera, N., Legge, S. E., Bishop, S., Cameron, D., Hamshere, M. L., et al. (2018). Common schizophrenia alleles are enriched in mutation-intolerant genes and in regions under strong background selection. Nat. Genet. 50, 381-389.
48. Pérez-Durán, P., Belver, L., de Yébenes, V. G., Delgado, P., Pisano, D. G., and Ramiro, A. R. (2012). UNG shapes the specificity of AID-induced somatic hypermutation. J. Exp. Med. 209, 1379-1389.
49. Pérez-Palma, E., Gramm, M., Nürnberg, P., May, P., and Lal, D. (2019). Simple ClinVar: an interactive web server to explore and retrieve gene and disease variants aggregated in ClinVar database. Nucleic Acids Res. 47, W99-W105.
50. Pham, P., Bransteitter, R., Petruska, J., and Goodman, M. F. (2003). Processive AID-catalysed cytosine deamination on single-stranded DNA simulates somatic hypermutation. Nature 424, 103-107.
51. Pham, P., Smolka, M. B., Calabrese, P., Landolph, A., Zhang, K., Zhou, H., and Goodman, M. F. (2008). Impact of phosphorylation and phosphorylation-null mutants on the activity and deamination specificity of activation-induced cytidine deaminase. J. Biol. Chem. 283, 17428-17439.
52. Rajagopal, N., Srinivasan, S., Kooshesh, K., Guo, Y., Edwards, M. D., Banerjee, B., Syed, T., Emons, B. J. M., Gifford, D. K., and Sherwood, R. I. (2016). High-throughput mapping of regulatory DNA. Nat. Biotechnol. 34, 167-174.
53. Rees, H. A., and Liu, D. R. (2018). Base editing: precision chemistry on the genome and transcriptome of living cells. Nat. Rev. Genet. 19, 770-788.
54. Ryu, S.-M., Koo, T., Kim, K., Lim, K., Baek, G., Kim, S.-T., Kim, H. S., Kim, D., Lee, H., Chung, E., et al. (2018). Adenine base editing in mouse embryos and an adult mouse model of Duchenne muscular dystrophy. Nat. Biotechnol. 36, 536.
55. Shalem, O., Sanjana, N. E., Hartenian, E., Shi, X., Scott, D. A., Mikkelsen, T. S., Heckl, D., Ebert, B. L., Root, D. E., Doench, J. G., et al. (2014). Genome-Scale CRISPR-Cas9 Knockout Screening in Human Cells. Science 343, 84.
56. Shen, M. W., Arbab, M., Hsu, J. Y., Worstell, D., Culbertson, S. J., Krabbe, O., Cassa, A., Liu, D. R., Gifford, D. K., and Sherwood, R. I. (2018). Predictable and precise template-free CRISPR editing of pathogenic variants. Nature 563, 646-651.
57. Sherwood, R. I., Hashimoto, T., O'Donnell, C. W., Lewis, S., Barkal, A. A., Van Hoff, J. P., Karun, V., Jaakkola, T., and Gifford, D. K. (2014). Discovery of directional and nondirectional pioneer transcription factors by modeling DNase profile magnitude and shape. Nat. Biotechnol. 32, 171-178.
58. Song, C. Q., Jiang, T., Richter, M., Rhym, L. H., Koblan, L. W., Zafra, M. P., Schatoff, E. M., Doman, J. L., Cao, Y., Dow, L. E., et al. (2019). Adenine base editing in an adult mouse model of tyrosinaemia. Nat. Biomed. Eng.
59. Stahl, E. A., Breen, G., Forstner, A. J., McQuillin, A., Ripke, S., Trubetskoy, V., Mattheisen, M., Wang, Y., Coleman, J. R. I., Gaspar, H. A., et al. (2019). Genome-wide association study identifies 30 loci associated with bipolar disorder. Nat. Genet. 51, 793-803.
60. Stenson, P. D., Mort, M., Ball, E. V, Shaw, K., Phillips, A. D., and Cooper, D. N. (2014). The Human Gene Mutation Database: Building a comprehensive mutation repository for clinical and molecular genetics, diagnostic testing and personalized genomic medicine. Hum. Genet. 133, 1-9.
61. Tan, J., Zhang, F., Karcher, D., and Bock, R. (2019). Engineering of high-precision base editors for site-specific single nucleotide replacement. Nat. Commun. 10.
62. Thuronyi, B. W., Koblan, L. W., Levy, J. M., Yeh, W.-H., Zheng, C., Newby, G. A., Wilson, C., Bhaumik, M., Shubina-Oleinik, O., Holt, J. R., et al. (2019). Continuous evolution of base editors with expanded target compatibility and improved activity. Nat. Biotechnol.
63. Urasaki, A., Morvan, G., and Kawakami, K. (2006). Functional dissection of the Tol2 transposable element identified the minimal cis-sequence and a highly repetitive sequence in the subterminal region essential for transposition. Genetics 174, 639-649.
64. Villiger, L., Grisch-Chan, H. M., Lindsay, H., Ringnalda, F., Pogliano, C. B., Allegri, G., Fingerhut, R., Haberle, J., Matos, J., Robinson, M. D., et al. (2018). Treatment of a metabolic liver disease by in vivo genome base editing in adult mice. Nat Med 24, 1519-1525.
65. Wang, T., Wei, J. J., Sabatini, D. M., and Lander, E. S. (2014). Genetic Screens in Human Cells Using the CRISPR-Cas9 System. Science 343, 80.
66. Yamane, A., Resch, W., Kuo, N., Kuchen, S., Li, Z., Sun, H. W., Robbiani, D. F., McBride, K., Nussenzweig, M. C., and Casellas, R. (2011). Deep-sequencing identification of the genomic targets of the cytidine deaminase AID and its cofactor RPA in B lymphocytes. Nat. Immunol. 12, 62-69.
67. Yeh, W. H., Chiang, H., Rees, H. A., Edge, A. S. B., and Liu, D. R. (2018). In vivo base editing of post-mitotic sensory cells. Nat. Commun. 9.
68. Zeng, Y., Li, J., Li, G., Huang, S., Yu, W., Zhang, Y., Chen, D., Chen, J., Liu, J., and Huang, X. (2018). Correction of the Marfan Syndrome Pathogenic FBN1 Mutation by Base Editing in Human Cells and Heterozygous Embryos. Mol. Ther. 26, 2631-2637.

### Example 2. Use of ABE8 at the SMN Exon 7 Locus to Edit the C:G→T:A SNV that is Causal to Spinal Muscular Atrophy (SMA)

This Example tested a series of ABE8-based editor to repair the C:G→T:A SNV mutation in position 6 of exon 7 that is causal to spinal muscular atrophy (SMA). Eleven different editors were tested, including ABE8 fusions to SpCas9, SaKKH, Cas9-NG, CP1028, CP1041, CP1041-NG, VRQR, Cpf1, SpyMac, and the evolved NRRH and NRCH editors developed by our lab in combination with a series of sgRNAs that placed the target nucleotide at positions ranging 5 through 12 in exon 7.

Fusion Constructs Tested:

| BASE EDITOR FUSION | AMINO ACID SEQUENCE |
|---|---|
| ABE8-NRTH editor | (SEQ ID NO: 463) |
| ABE8-SpyMac editor | (SEQ ID NO: 464) |
| ABE8-VRQR-CP1041 editor | (SEQ ID NO: 465) |
| ABE8-SaCas9 editor | (SEQ ID NO: 466) |
| ABE8-NRCH editor | (SEQ ID NO: 467) |
| ABE8-NRRH editor | (SEQ ID NO: 468) |
| ABE8-SaKKH editor | (SEQ ID NO: 469) |
| ABE8-Cas9-NG editor | (SEQ ID NO: 470) |
| ABE8-CP1041 editor | (SEQ ID NO: 471) |
| ABE8-CP1028 editor | (SEQ ID NO: 472) |
| ABE8-CPF1 editor | (SEQ ID NO: 473) |
| ABE8-VRQR editor | (SEQ ID NO: 474) |
| ABE8-Cas9-NG-CP1041 editor | (SEQ ID NO: 475) |
| ABE8-iSpyMac editor | (SEQ ID NO: 476) |

sgRNAs Tested for Each Construct:

| sgRNA | | SNV | |
|---|---|---|---|
| name | PAM | position | sequence |
| 3 | NGA | 9 | TTTTGTCTAAAACCctgtaa |
| | | | SEQ ID NO: 368) |
| 37 | NGG | 10 | ATTTTGTCTAAAACCctgta |
| | | | (SEQ ID NO: 364) |
| 139 | NNNRRT | 8 | TTTGTCTAAAACCctgtaag |
| | | | (SEQ ID NO: 366) |
| 52 | NAA | 8 | TTTGTCTAAAACCctgtaag |
| | | | (SEQ ID NO: 366) |
| 177 | NAT | 5 | GTCTAAAACCCTGTAAGGAA |
| | | | (SEQ ID NO: 408) |
| 178 | NAA | 6 | TGTCTAAAACCCTGTAAGGA |
| | | | (SEQ ID NO: 409) |
| 179 | NAA | 7 | TTGTCTAAAACCCTGTAAGG |
| | | | (SEQ ID NO: 410) |
| 54 | TTTV | 10 | ATTTTGTCTAAAACCCTGTAAGG |
| | | | (SEQ ID NO: 411) |
| 55 | TTTV | 11 | GATTTTGTCTAAAACCCTGTAAG |
| | | | (SEQ ID NO: 412) |
| 56 | TTTV | 12 | TGATTTTGTCTAAAACCCTGTAA |
| | | | (SEQ ID NO: 413) |

FIG. 4 shows the results of adenine base editing of the SMN2 disease causing SNV in SMA mESCs. Editors are denoted below the x-axis with PAM sequence in parentheses, and protospacer position of the target nucleotide assuming a 20nt protospacer where the PAM is at position 21-23. The results show that the iSpyMac and the CP constructs edited the SNV mutation with high efficiency.

Proof of repair of the exon 7 splicing error is shown in FIG. 5, which shows a gel electrophoresis image of SMN cDNA PCR amplification spanning exon 6 to exon 8, depicting bands that include or that have skipped exon 7 in pre-mRNA splicing in SMA mESCs treated with the indicated ABE8-fusion base editors.

### EQUIVALENTS AND SCOPE

In the claims articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention includes embodiments in which more than one, or all of the group members are present in, employed in, or otherwise relevant to a given product or process.

Furthermore, the invention encompasses all variations, combinations, and permutations in which one or more limitations, elements, clauses, and descriptive terms from one or more of the listed claims are introduced into another claim. For example, any claim that is dependent on another claim can be modified to include one or more limitations found in any other claim that is dependent on the same base claim. Where elements are presented as lists, e.g., in Markush group format, each subgroup of the elements is also disclosed, and any element(s) can be removed from the group. It should it be understood that, in general, where the invention, or aspects of the invention, is/are referred to as comprising particular elements and/or features, certain embodiments of the invention or aspects of the invention consist, or consist essentially of, such elements and/or features. For purposes of simplicity, those embodiments have not been specifically set forth in haec verba herein. It is also noted that the terms “comprising” and “containing” are intended to be open and permits the inclusion of additional elements or steps. Where ranges are given, endpoints are included. Furthermore, unless otherwise indicated or otherwise evident from the context and understanding of one of ordinary skill in the art, values that are expressed as ranges can assume any specific value or sub-range within the stated ranges in different embodiments of the invention, to the tenth of the unit of the lower limit of the range, unless the context clearly dictates otherwise.

This application refers to various issued patents, published patent applications, journal articles, and other publications, all of which are incorporated herein by reference. If there is a conflict between any of the incorporated references and the instant specification, the specification shall control. In addition, any particular embodiment of the present invention that falls within the prior art may be explicitly excluded from any one or more of the claims. Because such embodiments are deemed to be known to one of ordinary skill in the art, they may be excluded even if the exclusion is not set forth explicitly herein. Any particular embodiment of the invention can be excluded from any claim, for any reason, whether or not related to the existence of prior art.

What is claimed is:

1. A method of using at least one machine learning model to identify at least one guide RNA for use in a base editing system for introducing a desired change in a nucleotide sequence, the base editing system comprising a napDNAbp and a deaminase, the method comprising:
using software executing on at least one computer hardware processor to perform:
obtaining input data indicative of the nucleotide sequence and a set of one or more guide RNAs;
generating first input features from the input data;
applying a first machine learning model to the first input features to obtain first output data indicative, for each guide RNA in the set of guide RNAs, of a base editing efficiency, at one or multiple locations in the nucleotide sequence, of the base editing system when using the each guide RNA,
generating second input features from the input data;
applying a second machine learning model to the second input features to obtain second output data indicative, for each guide RNA in the set of guide RNAs, of bystander editing activity, at one or multiple locations in the nucleotide sequence, by the base editing system when using the each guide RNA; and
identifying, using the first output data and the second output data, the at least one guide RNA for use in the base editing system for introducing the desired change in the nucleotide sequence.

2. The method of claim 1, wherein the set of guide RNAs includes a first guide RNA, and wherein, the input data includes first data indicative of at least a part of a nucleotide sequence associated with the first guide RNA.

3. The method of claim 2, wherein the first data specifies a spacer or a protospacer sequence associated with the first guide RNA.

4. The method of claim 1 or any other preceding claim, wherein obtaining the input data indicative of the nucleotide sequence and the set of guide RNAs, comprises:
obtaining, by the software and from at least one source external to the software, the input data indicative of the nucleotide sequence and the set of guide RNAs.

5. The method of claim 1 or any other preceding claim, wherein obtaining the data indicative of the nucleotide sequence and the set of guide RNAs, comprises:
obtaining, by the software and from at least one source external to the software, first data indicative of the nucleotide sequence; and
generating, from the first data indicative of the nucleotide sequence, data indicative of the set of guide RNAs.

6. The method of claim 1 or any other preceding claim, wherein the first machine learning model comprises a non-linear machine learning model selected from the group consisting of a random forest model, a logistic regression model, a support vector machine model, a generalized linear model, a hierarchical Bayesian model, and neural network model.

7. The method of claim 1 or any other preceding claim, wherein the first machine learning model comprises a random forest model.

8. The method of claim 1 or any other preceding claim, wherein the set of guide RNAs includes a first guide RNA, and wherein generating the first input features comprises generating multiple features to include in the first input features, the multiple features including:
features encoding at least some nucleotides in a protospacer sequence or spacer sequence associated with the first guide RNA; and
features encoding at least some nucleotides, in the nucleotide sequence, located within a threshold number of nucleotides of the protospacer sequence associated with the first guide RNA.

9. The method of claim 8, wherein generating the features encoding the at least some nucleotides in the protospacer sequence comprises generating a one-hot encoding of the at least some nucleotides in the protospacer sequence.

10. The method of claim 8, wherein the multiple features further include one or more of the following features:
features encoding at least some dinucleotides at neighboring positions in the protospacer sequence;
features representing melting temperature of the first guide RNA;
one or more features representing a total number of G, C, A, and/or T nucleotides in the protospacer sequence;
one or more features representing a percentage of G, C, A, and/or T nucleotides in the protospacer sequence; and
a feature representing an average base editing efficiency of the base editing system.

11. The method of claim 1 or any other preceding claim, wherein the set of guide RNAs includes a first guide RNA, wherein the first output data is indicative of a fraction of sequence reads containing at least one base edit at any nucleotide in a desired window about a protospacer sequence associated with the first guide RNA, among all sequence reads.

12. The method of claim 1 or any other preceding claim, wherein the second first machine learning model comprises a non-linear machine learning model selected from the group consisting of a random forest model, a logistic regression model, a support vector machine model, a generalized linear model, a hierarchical Bayesian model, and neural network model.

13. The method of claim 12 or any other preceding claim, wherein the second machine learning model comprises a deep neural network model.

14. The method of claim 13, wherein the neural network model comprises a conditional autoregressive neural network model.

15. The method of claim 14, wherein the conditional autoregressive neural network model includes:
an encoder neural network mapping input data to a latent representation; and
a decoder neural network mapping the latent representation to output data,
wherein the decoder neural network has an autoregressive structure.

16. The method of claim 15, wherein the encoder neural network comprises a multi-layer fully connected network with residual connections.

17. The method of claim 15, wherein the decoder neural network generates a distribution over base editing outcomes at each nucleotide while conditioning on previously-generated outcomes.

18. The method of claim 13, wherein the neural network model includes parameters representing a position-wise bias toward producing an unedited outcome.

19. The method of claim 1 or any other preceding claim, wherein the set of guide RNAs includes a first guide RNA, and wherein generating the second input features comprises generating multiple features to include in the second input features, the multiple features including:
features encoding at least some nucleotides in a protospacer sequence or spacer sequence associated with the first guide RNA; and
features encoding at least some nucleotides, in the nucleotide sequence, located within a threshold number of nucleotides of the protospacer sequence associated with the first guide RNA.

20. The method of claim 1 or any other preceding claim, wherein the second output data is indicative of frequencies of occurrence of base editing outcomes, each of which includes edits to nucleotides at multiple positions.

21. The method of claim 1 or any other preceding claim, wherein the second output data is indicative of a frequency distribution on combinations of base editing outcomes.

22. The method of claim 1 or any other preceding claim, wherein the set of guide RNAs includes a first guide RNA, wherein, for a specific combination of base edits, the second output data is indicative of a frequency of occurrence of the specific combination of base edits among all sequenced reads containing at least one base edit at any nucleotide in a desired window about a protospacer sequence associated with the first guide RNA.

23. The method of claim 1, wherein the set of guide RNAs includes a first guide RNA, wherein the first output data includes a first base editing efficiency value for the first guide RNA, wherein the second output data includes a first bystander editing value for the first guide RNA, and wherein identifying the guide RNA using the first output data and the second output data, comprises multiplying the first base editing efficiency value by the first bystander editing value.

24. The method of claim 1 or any other preceding claim, wherein the first machine learning model comprises a first plurality of values for a respective first plurality of parameters, the first plurality of values used by the at least one computer hardware processor to obtain the first output data from the first input features.

25. The method of claim 24 or any other preceding claim, wherein the first plurality of parameters comprises at least one thousand parameters.

26. The method of claim 25, wherein the first plurality of parameters comprises between one thousand and ten thousand parameters.

27. The method of claim 24 or any other preceding claim, wherein the first machine learning model comprises a random forest model comprising at least 100 decision trees, each of the at least 100 decision trees having at least a depth of D, and wherein processing the input data using the random forest model comprises performing 100*D comparisons.

28. The method of claim 27, wherein the random forest model comprises at least 500 decision trees.

29. The method of claim 27, wherein D is greater than or equal to five, wherein processing the input data using the random forest model comprises performing at least 2500 comparisons.

30. The method of claim 1 or any other preceding claim, wherein the second machine learning model comprises a second plurality of values for a respective second plurality of parameters, the second plurality of values used by the at least one computer hardware processor to obtain the second output data from the second input features.

31. The method of claim 30, wherein the second plurality of parameters comprises at least ten thousand parameters.

32. The method of claim 30, wherein the second plurality of parameters comprises between 25,000 and 100,000 parameters.

33. The method of claim 30, wherein the second plurality of parameters comprises between 30,000 and 40,000 parameters.

34. The method of claim 1 or any other preceding claim further comprising:
synthesizing the identified guide RNA for use in the base editing system for introducing the desired change in the nucleotide sequence.

35. The method of claim 1 or any other preceding claim further comprising:
using the identified guide RNA and the base editing system to introduce the desired change in a cell.

36. The method of claim 1 or any other preceding claim further comprising:
determining a likelihood of whether the identified guide RNA and the base editing system, when used in combination, will result in introducing the desired change in a cell.

37. A method for training the first machine learning model of any of claims 1-36, comprising: (i) preparing a library comprising a plurality of nucleic acid molecules each encoding a nucleotide desired sequence and a cognate guide RNA; (ii) introducing the library into a plurality of host cells; (iii) contacting the library in the host cells with a Cas-based genome editing system to produce a plurality of genomic repair products; (iv) determining the sequences of the genomic repair products; and (v) training the first machine learning model with training data that comprises at least the sequences of the genomic repair products and the cognate guide RNA.

38. A method for training the second machine learning model of any of claims 1-36, comprising: (i) preparing a library comprising a plurality of nucleic acid molecules each encoding a nucleotide desired sequence and a cognate guide RNA; (ii) introducing the library into a plurality of host cells; (iii) contacting the library in the host cells with a Cas-based genome editing system to produce a plurality of genomic repair products; (iv) determining the sequences of the genomic repair products; and (v) training the second machine learning model with training data that comprises at least the sequences of the genomic repair products and the cognate guide RNA.

39. At least one computer readable storage medium storing processor executable instructions that, when executed by at least one computer hardware processor, cause the at least one computer hardware processor to perform a method of using at least one machine learning model to identify at least one guide RNA for use in a base editing system for introducing a desired change in a nucleotide sequence, the base editing system comprising a napDNAbp and a deaminase, the method comprising:
obtaining input data indicative of the nucleotide sequence and a set of one or more guide RNAs;
generating first input features from the input data;
applying a first machine learning model to the first input features to obtain first output data indicative, for each guide RNA in the set of guide RNAs, of a base editing efficiency, at one or multiple locations in the nucleotide sequence, of the base editing system when using the each guide RNA;
generating second input features from the input data;
applying a second machine learning model to the second input features to obtain second output data indicative, for each guide RNA in the set of guide RNAs, of bystander editing activity, at one or multiple locations in the nucleotide sequence, by the base editing system when using the each guide RNA; and
identifying, using the first output data and the second output data, the at least one guide RNA for use in the base editing system for introducing the desired change in the nucleotide sequence.

40. A system comprising:
at least one computer hardware processor; and
at least one computer readable storage medium storing processor executable instructions that, when executed by the at least one computer hardware processor, cause the at least one computer hardware processor to perform a method of using at least one machine learning model to identify at least one guide RNA for use in a base editing system for introducing a desired change in a nucleotide sequence, the base editing system comprising a napDNAbp and a deaminase, the method comprising:
obtaining input data indicative of the nucleotide sequence and a set of one or more guide RNAs;
generating first input features from the input data;
applying a first machine learning model to the first input features to obtain first output data indicative, for each guide RNA in the set of guide RNAs, of a base editing efficiency, at one or multiple locations in the nucleotide sequence, of the base editing system when using the each guide RNA;
generating second input features from the input data;
applying a second machine learning model to the second input features to obtain second output data indicative, for each guide RNA in the set of guide RNAs, of bystander editing activity, at one or multiple locations in the nucleotide sequence, by the base editing system when using the each guide RNA; and
identifying, using the first output data and the second output data, the at least one guide RNA for use in the base editing system for introducing the desired change in the nucleotide sequence.

41. A method of identifying at least one guide RNA for use in a base editing system for introducing a desired change in a nucleotide sequence, the base editing system comprising a napDNAbp and a deaminase, the method comprising:
using software executing on at least one computer hardware processor to perform:
obtaining input data indicative of the nucleotide sequence and a set of one or more guide RNAs;
generating first input features from the input data;
applying a first machine learning model to the first input features to obtain first output data indicative, for each guide RNA in the set of guide RNAs, of a base editing efficiency, at one or multiple locations in the nucleotide sequence, of the base editing system when using the each guide RNA; and
identifying, using the first output data, the at least one guide RNA for use in the base editing system for introducing the desired change in the nucleotide sequence.

42. The method of claim 41, further comprising:
generating second input features from the input data;
applying a second machine learning model to the second input features to obtain second output data indicative, for each guide RNA in the set of guide RNAs, of bystander editing activity, at one or multiple locations in the nucleotide sequence, by the base editing system when using the each guide RNA,
wherein identifying the guide RNA is performed using the first output data and the second output data.

43. At least one computer readable storage medium storing processor executable instructions that, when executed by at least one computer hardware processor, cause the at least one computer hardware processor to perform a method of identifying at least one guide RNA for use in a base editing system for introducing a desired change in a nucleotide sequence, the base editing system comprising a napDNAbp and a deaminase, the method comprising:
obtaining input data indicative of the nucleotide sequence and a set of one or more guide RNAs;
generating first input features from the input data;
applying a first machine learning model to the first input features to obtain first output data indicative, for each guide RNA in the set of guide RNAs, of a base editing efficiency, at one or multiple locations in the nucleotide sequence, of the base editing system when using the each guide RNA; and
identifying, using the first output data, the at least one guide RNA for use in the base editing system for introducing the desired change in the nucleotide sequence.

44. A system, comprising:
at least one computer hardware processor; and
at least one computer readable storage medium storing processor executable instructions that, when executed by the at least one computer hardware processor, cause the at least one computer hardware processor to perform a method of identifying at least one guide RNA for use in a base editing system for introducing a desired change in a nucleotide sequence, the base editing system comprising a napDNAbp and a deaminase, the method comprising:
obtaining input data indicative of the nucleotide sequence and a set of one or more guide RNAs;
generating first input features from the input data;
applying a first machine learning model to the first input features to obtain first output data indicative, for each guide RNA in the set of guide RNAs, of a base editing efficiency, at one or multiple locations in the nucleotide sequence, of the base editing system when using the each guide RNA; and
identifying, using the first output data, the at least one guide RNA for use in the base editing system for introducing the desired change in the nucleotide sequence.

45. A method of identifying at least one guide RNA for use in a base editing system for introducing a desired change in a nucleotide sequence, the base editing system comprising a napDNAbp and a deaminase, the method comprising:
using software executing on at least one computer hardware processor to perform:
obtaining input data indicative of the nucleotide sequence and a set of one or more guide RNAs;
generating first input features from the input data;
applying a first machine learning model to the first input features to obtain first output data indicative, for each guide RNA in the set of guide RNAs, of bystander editing activity, at one or multiple locations in the nucleotide sequence, by the base editing system when using the each guide RNA; and
identifying, using the first output data, the at least one guide RNA for use in the base editing system for introducing the desired change in the nucleotide sequence.

46. The method of claim 45, further comprising:
generating second input features from the input data;
applying a second machine learning model to the second input features to obtain second output data indicative, for each guide RNA in the set of guide RNAs, of a base editing efficiency, at one or multiple locations in the nucleotide sequence, of the base editing system when using the each guide RNA,
wherein identifying the guide RNA is performed using the first output data and the second output data.

47. At least one computer readable storage medium storing processor executable instructions that, when executed by at least one computer hardware processor, cause the at least one computer hardware processor to perform a method of identifying at least one guide RNA for use in a base editing system for introducing a desired change in a nucleotide sequence, the base editing system comprising a napDNAbp and a deaminase, the method comprising:
obtaining input data indicative of the nucleotide sequence and a set of one or more guide RNAs;
generating first input features from the input data;
applying a first machine learning model to the first input features to obtain first output data indicative, for each guide RNA in the set of guide RNAs, of bystander editing activity, at one or multiple locations in the nucleotide sequence, by the base editing system when using the each guide RNA; and
identifying, using the first output data, the at least one guide RNA for use in the base editing system for introducing the desired change in the nucleotide sequence.

48. A system, comprising:
at least one computer hardware processor; and
at least one computer readable storage medium storing processor executable instructions that, when executed by at least one computer hardware processor, cause the at least one computer hardware processor to perform a method of identifying at least one guide RNA for use in a base editing system for introducing a desired change in a nucleotide sequence, the base editing system comprising a napDNAbp and a deaminase, the method comprising:
obtaining input data indicative of the nucleotide sequence and a set of one or more guide RNAs;
generating first input features from the input data;
applying a first machine learning model to the first input features to obtain first output data indicative, for each guide RNA in the set of guide RNAs, of bystander editing activity, at one or multiple locations in the nucleotide sequence, by the base editing system when using the each guide RNA; and
identifying, using the first output data, the at least one guide RNA for use in the base editing system for introducing the desired change in the nucleotide sequence.

49. A method, comprising:
using software executing on at least one computer hardware processor to perform:
receiving input data indicative of a selection of:
a nucleotide sequence;
a base editing system comprising a napDNAbp and a deaminase; and
a first guide RNA;
applying a first machine learning model to the first input features, generated from the input data, to obtain first output data indicative of a base editing efficiency, at a desired location in the nucleotide sequence, of the base editing system when using the first guide RNA;
applying a second machine learning model to the second input features, generated from the input data, to obtain second output data indicative of bystander editing activity, at one or multiple locations in the nucleotide sequence, by the base editing system when using the first guide RNA; and
determining, using the first output data and the second output data, a likelihood of whether the first guide RNA and the base editing system, when used in combination, will result in introduce a desired change to the nucleotide sequence in a cell.

50. At least one computer-readable storage medium storing processor-executable instructions that, when executed by at least one computer hardware processor, cause the at least one computer processor to perform:
receiving input data indicative of a selection of:
a nucleotide sequence;
a base editing system comprising a napDNAbp and a deaminase; and
a first guide RNA;
applying a first machine learning model to the first input features, generated from the input data, to obtain first output data indicative of a base editing efficiency, at a desired location in the nucleotide sequence, of the base editing system when using the first guide RNA;
applying a second machine learning model to the second input features, generated from the input data, to obtain second output data indicative of bystander editing activity, at one or multiple locations in the nucleotide sequence, by the base editing system when using the first guide RNA; and
determining, using the first output data and the second output data, a likelihood of whether the first guide RNA and the base editing system, when used in combination, will result in introduce a desired change to the nucleotide sequence in a cell.

51. A system, comprising:
at least one computer hardware processor; and
at least one computer-readable storage medium storing processor-executable instructions that, when executed by the at least one computer hardware processor, cause the at least one computer processor to perform:
receiving input data indicative of a selection of:
a nucleotide sequence;
a base editing system comprising a napDNAbp and a deaminase; and
a first guide RNA;
applying a first machine learning model to the first input features, generated from the input data, to obtain first output data indicative of a base editing efficiency, at a desired location in the nucleotide sequence, of the base editing system when using the first guide RNA;
applying a second machine learning model to the second input features, generated from the input data, to obtain second output data indicative of bystander editing activity, at one or multiple locations in the nucleotide sequence, by the base editing system when using the first guide RNA; and
determining, using the first output data and the second output data, a likelihood of whether the first guide RNA and the base editing system, when used in combination, will result in introduce a desired change to the nucleotide sequence in a cell.

52. A guide RNA for use in a base editing system for introducing a target change into a target DNA sequence identified by the method of any of claims 1-51.

53. A guide RNA comprising a protospacer selected from the group consisting of SEQ ID Nos: 451-3199.

54. The guide RNA of any of claims 52-53, wherein at least one base editor demonstrated at least 50% correction precision to the wild-type genotype among edited reads.

55. The guide RNA of any of claims 52-54, wherein the least one base editor is ABE (SEQ ID NO: 3210), ABE-CP1041 (SEQ ID NO: 3211), AID-BE4 (SEQ ID NO: 3202), BE4 (SEQ ID NO: 3200), BE4-CP1028 (SEQ ID NO: 3208), CDA-BE4 (SEQ ID NO: 3203), eA3A-BE4 (SEQ ID NO: 3205), eA3A_T31AT44A, or evoAPOBEC1-BE4max (SEQ ID NO: 3204).

56. The guide RNA of any of claims 52-55, wherein the base editing system comprises an ABE of SEQ ID NO: 3210 and said guide RNA comprises a protospacer identified as any of the sequences of Index Nos. 880-2498 of Table 5.

57. The guide RNA of any of claims 52-56, wherein the base editing system comprises an ABE-CP1041 of SEQ ID NO: 3211, and said guide RNA comprises a protospacer identified as any of the sequences of Index Nos. 880-990, 998-1014, 1042-1313, 1749-2184, 2186-2695 of Table 5.

58. The guide RNA of any of claims 52-57, wherein the base editing system comprises an AID-BE4 of SEQ ID NO: 3202, and said guide RNA comprises a protospacer identified as any of the sequences of Index Nos. 1-301 of Table 5.

59. The guide RNA of any of claims 52-58, wherein the base editing system comprises an BE4 of SEQ ID NO: 3200, and said guide RNA comprises a protospacer identified as any of the sequences of Index Nos. 2-3, 6-12, 16-17, 19-27, 40-42, 44, 47-48, 52-53, 55-58, 62-65, 68, 70, 74-78, 80, 82-92, 94-98, 198, 200-204, 207, 210-211, 213-219, 222-224, 226-229, 231-233, 235-236, 238, 244, 247-248, 252-255, 257-258, 260, 263-270, 272-275, 279, 281-287, 289-290, 293-294, 296, 298-299, 301, 541, 543-626, 628-712, 722-723, 798-838, 840-848, 858-878 of Table 5.

60. The guide RNA of any of claims 52-59, wherein the base editing system comprises an BE4-CP1028 of SEQ ID NO: 3208, and said guide RNA comprises a protospacer identified as any of the sequences of Index Nos. 2-3, 5-9, 11-15, 17-27, 40, 42, 44, 47-50, 52-54, 56-58, 63, 65, 74-75, 77, 79-83, 85, 87-93, 96-98, 157, 162, 182, 263, 302, 305, 308, 313, 315, 324, 336, 338, 341, 343, 345, 403, 407-411, 413, 415-416, 418-419, 421, 423-427, 429-440, 461-464, 467-468, 470-471, 473, 508-514, 516-520, 522-524, 526-535, 537, 539-540, 544, 586, 588-590, 592-605, 607, 621, 624, 632, 702-703, 705-708, 710-712, 723, 799-801, 803-804, 807-808, 810, 813-816, 818-828, 830-835, 837-838, 840-848, 858-860, 864-873, 876-878 of Table 5.

61. The guide RNA of any of claims 52-60, wherein the base editing system comprises an CDA-BE4 of SEQ ID NO: 3203, and said guide RNA comprises a protospacer identified as any of the sequences of Index Nos. 4, 6-7, 9-13, 15-17, 20-24, 26, 31-32, 35, 40-41, 44, 47-50, 52-53, 55, 63-65, 68, 70-72, 75-81, 84-87, 89-94, 98, 100-101, 103-104, 107, 109, 111, 113, 118-121, 124-127, 130-132, 136, 141-144, 146-148, 151-160, 162, 164, 166-167, 170, 172-173, 175-180, 184, 195, 198, 200-204, 206-215, 218-219, 221-224, 226-227, 230, 233-234, 237, 239, 243-244, 247, 251-257, 261-267, 274, 281-284, 286-287, 289-290, 292, 295, 297-302, 304, 411-412, 414, 417, 420, 422-423, 425, 428, 431, 433, 435, 438, 442-445, 457, 463, 472, 477-479, 485, 488, 491, 493-494, 507, 510, 513, 515, 518, 521, 536, 538, 540, 542, 552, 561, 563-569, 573-582, 587-588, 591, 593-595, 598, 622-623, 625, 627, 640, 667, 704, 712-721, 724-727, 734-752, 755, 759, 761-768, 773-774, 776, 780, 785-786, 788-789, 795-797, 800, 802, 805-806, 811-812, 814, 817-818, 820, 829, 831, 833, 835, 839-842, 849, 852, 854, 856, 861, 864, 874-875, 878-879 of Table 5.

62. The guide RNA of any of claims 52-61, wherein the base editing system comprises an eA3A-BE4 of SEQ ID NO: 3204, and said guide RNA comprises a protospacer identified as any of the sequences of Index Nos. 2-3, 6, 8-10, 13, 15-17, 20, 22-23, 25, 27-28, 32, 35, 42, 45-47, 53, 55-56, 63-64, 74, 76, 80-81, 86-92, 96-98, 111, 119, 121, 127, 151, 154, 156, 159-160, 171, 178, 180, 184, 192, 198, 204-206, 210-211, 214, 216-217, 220, 224, 228-229, 231-233, 235, 244, 247, 252-253, 260, 263-268, 270, 272-274, 276, 279, 281-285, 287-289, 293-294, 296, 298, 303-304, 306-312, 314, 316-317, 319-323, 326-329, 331-337, 339, 343-345, 347-348, 352-362, 364-372, 374-406, 410-411, 432-434, 438, 446-447, 449-453, 456, 458, 460, 466, 468-469, 474-476, 481, 486, 489-490, 492, 495-506, 521, 523, 525, 539, 543-551, 553-556, 558-564, 569, 573, 575, 578-579, 581, 583-584, 588, 590, 593, 595-596, 598-600, 602, 604, 607, 614-620, 622, 624, 626, 628-630, 632-639, 641-647, 651, 657, 660, 662-663, 665-666, 668-671, 673-674, 678, 686-689, 691-693, 695-700, 702-703, 707-709, 711-712, 715, 723, 741, 800-806, 808, 811, 813-821, 823-827, 829-830, 832-833, 835, 844, 846-849, 852, 858-860, 865-866, 868-870, 872-874, 878, 2696-2737 of Table 5.

63. The guide RNA of any of claims 52-62, wherein the base editing system comprises an eA3A_T31AT44A of SEQ ID NO: 3206, and said guide RNA comprises a protospacer identified as any of the sequences of Index Nos. 2725-2726 and 2738-2749 of Table 5.

64. The guide RNA of any of claims 52-63, wherein the base editing system comprises an evoAPOBEC1-BE4max of SEQ ID NO: 3204, and said guide RNA comprises a protospacer identified as any of the sequences of Index Nos. 1-4, 6-7, 9-11, 13, 15-18, 20, 22-27, 32, 35, 40-42, 44, 47-49, 51-53, 55-56, 58, 61-63, 68, 70-72, 74, 76-82, 84-92, 94-98, 100, 104, 108, 111, 116, 121, 125-126, 131, 136, 141-143, 146-148, 150-151, 153, 155-160, 162, 170, 172, 175, 178-180, 183-184, 190, 195, 198, 200-201, 203-204, 206, 210-212, 214, 217, 220-221, 223-227, 229, 231-233, 235-239, 244, 247, 249, 252-258, 263-270, 272-274, 276, 278-279, 281-284, 286-290, 293-294, 296, 298, 300-301, 304, 318, 321, 324-325, 330-333, 338, 340, 342, 346, 349-351, 358, 363, 373, 379-380, 385-389, 411, 423, 425, 427, 431, 433, 438, 441, 445, 448, 454-455, 459, 463, 465, 472, 476, 480, 482-484, 487, 491, 493-494, 503, 510, 514, 517, 521, 535, 540, 542, 544-545, 551-555, 558-564, 567-568, 573-576, 579-582, 588-589, 593, 595-596, 598, 600, 603, 605, 610, 612-617, 620, 622, 625-626, 628, 630-631, 635-641, 644, 651, 653-654, 656, 676, 678-679, 682, 688, 694, 704, 711, 713-715, 717, 720-723, 728-734, 742-743, 745, 747, 750, 752-754, 756-758, 760, 762, 766, 769-773, 775, 777-779, 781-784, 787, 790-794, 798, 800, 803, 805-806, 809, 811-812, 814, 818-819, 824-825, 827, 829, 831, 833, 835, 838-839, 841-842, 847, 850-855, 857-859, 861, 864, 870-873, 875, 878-879 of Table 5.

65. A complex comprising a base editor and a guide RNA selected from the method of claim 1 or a guide RNA of any one of claims 52-64.

66. The complex of claim 65, wherein the base editor comprises a napDNAbp.

67. The complex of claim 66, wherein the napDNAbp is a Cas9 or variant thereof.

68. The complex of claim 66, wherein the napDNAbp is a wildtype SpCas9 comprising an amino acid sequence of SEQ ID NO: 5, or an amino acid sequence having at least 80%, 85%, 90%, 95%, or 99% sequence identity with SEQ ID NO: 5.

69. The complex of claim 66, wherein the napDNAbp is a wildtype SpCas9 comprising an amino acid sequence of SEQ ID NOs: 5, 8, 10, 12, and 407 or an amino acid sequence having at least 80%, 85%, 90%, 95%, or 99% sequence identity with any one of SEQ ID NOs: 5, 8, 10, 12, or 407.

70. The complex of claim 66, wherein the napDNAbp is a SpCas9 ortholog or homolog comprising an amino acid sequence of SEQ ID Nos: 13-26, 44-63, or 74-77, or an amino acid sequence having at least 80%, 85%, 90%, 95%, or 99% sequence identity with any one of SEQ ID NOs: 13-26, 44-63, or 74-77.

71. The complex of claim 66, wherein the napDNAbp is a dead Cas9 comprising an amino acid sequence of SEQ ID Nos: 27-28, or an amino acid sequence having at least 80%, 85%, 90%, 95%, or 99% sequence identity with any one of SEQ ID NOs: 27-28.

72. The complex of claim 66, wherein the napDNAbp is a nickase Cas9 comprising an amino acid sequence of SEQ ID Nos: 29-44, or an amino acid sequence having at least 80%, 85%, 90%, 95%, or 99% sequence identity with any one of SEQ ID NOs: 29-44.

73. The complex of claim 66, wherein the napDNAbp is a circular permutant variant of Cas9 comprising an amino acid sequence of SEQ ID Nos: 64-73, or an amino acid sequence having at least 80%, 85%, 90%, 95%, or 99% sequence identity with any one of SEQ ID NOs: 64-73.

74. The complex of claim 65, wherein the base editor comprises an adenine deaminase.

75. The complex of claim 65, wherein the base editor comprises a cytidine deaminase.

76. The complex of claim 74, wherein the adenine deaminase comprises an amino acid sequence of any one of SEQ ID NOs: 78-91, 403, or 462, or an amino acid sequence having at least 80%, 85%, 90%, 95%, or 99% sequence identity with any one of SEQ ID NOs: 78-91, 403, or 462.

77. The complex of claim 75, wherein the cytidine deaminase comprises an amino acid sequence of any one of SEQ ID NOs: 92-134, or an amino acid sequence having at least 80%, 85%, 90%, 95%, or 99% sequence identity with any one of SEQ ID NOs: 92-134.

78. The complex of claim 65, wherein the base editor comprises one or more linkers having an amino acid sequence comprising any one of SEQ ID NOs.: 135-151, or an amino acid sequence having at least 80%, 85%, 90%, 95%, or 99% sequence identity with any one of SEQ ID NOs: 135-151.

79. The complex of claim 65, wherein the base editor comprises one or more NLS having an amino acid sequence comprising any one of SEQ ID NOs.: 152-162, or an amino acid sequence having at least 80%, 85%, 90%, 95%, or 99% sequence identity with any one of SEQ ID NOs: 152-162.

80. The complex of claim 65, wherein the base editor comprises one or more UGI having an amino acid sequence comprising SEQ ID NO.: 163, or an amino acid sequence having at least 80%, 85%, 90%, 95%, or 99% sequence identity with SEQ ID NO:163.

81. The complex of claim 65, wherein the base editor is an adenosine base editor comprising an amino acid sequence of any one of SEQ ID NOs: 174-221 or 463-476, or an amino acid sequence having at least 80%, 85%, 90%, 95%, or 99% sequence identity with any one of SEQ ID NOs: 174-221 or 463-476.

82. The complex of claim 65, wherein the base editor is a cytidine base editor comprising an amino acid sequence of any one of SEQ ID NOs: 223-248, or an amino acid sequence having at least 80%, 85%, 90%, 95%, or 99% sequence identity with any one of SEQ ID NOs: 223-248.

83. The complex of claim 65, wherein the base editor is ABE (SEQ ID NO: 3210), ABE-CP1041 (SEQ ID NO: 3211), AID-BE4 (SEQ ID NO: 3202), BE4 (SEQ ID NO: 3200), BE4-CP1028 (SEQ ID NO: 3208), CDA-BE4 (SEQ ID NO: 3203), eA3A-BE4 (SEQ ID NO: 3205), eA3A_T31AT44A (SEQ ID NO: 3206), or evoAPOBEC1-BE4max (SEQ ID NO: 3204), or an amino acid sequence having at least 80%, 85%, 90%, 95%, or 99% sequence identity with any one of SEQ ID NOs: 3210, 3211, 3202, 3200, 3208, 3203, 3205, 3206, or 3204.

84. The complex of claim 65, wherein the guide RNA comprises a spacer corresponding to any one of the protospacers of SEQ ID Nos: 451-3199.

85. The complex of claim 65, wherein the base editing system comprises an ABE of SEQ ID NO: 3210 and said guide RNA comprises a spacer corresponding to any one of the protospacers identified as a sequence of Index Nos. 880-2498 of Table 5.

86. The complex of claim 65, wherein the base editing system comprises an ABE-CP1041 of SEQ ID NO: 3211, said guide RNA comprises a spacer corresponding to any one of the protospacers identified as a sequence of Index Nos. 880-990, 998-1014, 1042-1313, 1749-2184, 2186-2695 of Table 5.

87. The complex of claim 65, wherein the base editing system comprises an AID-BE4 of SEQ ID NO: 3202, said guide RNA comprises a spacer corresponding to any one of the protospacers identified as a sequence of Index Nos. 1-301 of Table 5.

88. The complex of claim 65, wherein the base editing system comprises an BE4 of SEQ ID NO: 3200, said guide RNA comprises a spacer corresponding to any one of the protospacers identified as a sequence of Index Nos. 2-3, 6-12, 16-17, 19-27, 40-42, 44, 47-48, 52-53, 55-58, 62-65, 68, 70, 74-78, 80, 82-92, 94-98, 198, 200-204, 207, 210-211, 213-219, 222-224, 226-229, 231-233, 235-236, 238, 244, 247-248, 252-255, 257-258, 260, 263-270, 272-275, 279, 281-287, 289-290, 293-294, 296, 298-299, 301, 541, 543-626, 628-712, 722-723, 798-838, 840-848, 858-878 of Table 5.

89. The complex of claim 65, wherein the base editing system comprises an BE4-CP1028 of SEQ ID NO: 3208, said guide RNA comprises a spacer corresponding to any one of the protospacers identified as a sequence of Index Nos. 2-3, 5-9, 11-15, 17-27, 40, 42, 44, 47-50, 52-54, 56-58, 63, 65, 74-75, 77, 79-83, 85, 87-93, 96-98, 157, 162, 182, 263, 302, 305, 308, 313, 315, 324, 336, 338, 341, 343, 345, 403, 407-411, 413, 415-416, 418-419, 421, 423-427, 429-440, 461-464, 467-468, 470-471, 473, 508-514, 516-520, 522-524, 526-535, 537, 539-540, 544, 586, 588-590, 592-605, 607, 621, 624, 632, 702-703, 705-708, 710-712, 723, 799-801, 803-804, 807-808, 810, 813-816, 818-828, 830-835, 837-838, 840-848, 858-860, 864-873, 876-878 of Table 5.

90. The complex of claim 65, wherein the base editing system comprises an CDA-BE4 of SEQ ID NO: 3203, said guide RNA comprises a spacer corresponding to any one of the protospacers identified as a sequence of Index Nos. 4, 6-7, 9-13, 15-17, 20-24, 26, 31-32, 35, 40-41, 44, 47-50, 52-53, 55, 63-65, 68, 70-72, 75-81, 84-87, 89-94, 98, 100-101, 103-104, 107, 109, 111, 113, 118-121, 124-127, 130-132, 136, 141-144, 146-148, 151-160, 162, 164, 166-167, 170, 172-173, 175-180, 184, 195, 198, 200-204, 206-215, 218-219, 221-224, 226-227, 230, 233-234, 237, 239, 243-244, 247, 251-257, 261-267, 274, 281-284, 286-287, 289-290, 292, 295, 297-302, 304, 411-412, 414, 417, 420, 422-423, 425, 428, 431, 433, 435, 438, 442-445, 457, 463, 472, 477-479, 485, 488, 491, 493-494, 507, 510, 513, 515, 518, 521, 536, 538, 540, 542, 552, 561, 563-569, 573-582, 587-588, 591, 593-595, 598, 622-623, 625, 627, 640, 667, 704, 712-721, 724-727, 734-752, 755, 759, 761-768, 773-774, 776, 780, 785-786, 788-789, 795-797, 800, 802, 805-806, 811-812, 814, 817-818, 820, 829, 831, 833, 835, 839-842, 849, 852, 854, 856, 861, 864, 874-875, 878-879 of Table 5.

91. The complex of claim 65, wherein the base editing system comprises an eA3A-BE4 of SEQ ID NO: 3204, said guide RNA comprises a spacer corresponding to any one of the protospacers identified as a sequence of Index Nos. 2-3, 6, 8-10, 13, 15-17, 20, 22-23, 25, 27-28, 32, 35, 42, 45-47, 53, 55-56, 63-64, 74, 76, 80-81, 86-92, 96-98, 111, 119, 121, 127, 151, 154, 156, 159-160, 171, 178, 180, 184, 192, 198, 204-206, 210-211, 214, 216-217, 220, 224, 228-229, 231-233, 235, 244, 247, 252-253, 260, 263-268, 270, 272-274, 276, 279, 281-285, 287-289, 293-294, 296, 298, 303-304, 306-312, 314, 316-317, 319-323, 326-329, 331-337, 339, 343-345, 347-348, 352-362, 364-372, 374-406, 410-411, 432-434, 438, 446-447, 449-453, 456, 458, 460, 466, 468-469, 474-476, 481, 486, 489-490, 492, 495-506, 521, 523, 525, 539, 543-551, 553-556, 558-564, 569, 573, 575, 578-579, 581, 583-584, 588, 590, 593, 595-596, 598-600, 602, 604, 607, 614-620, 622, 624, 626, 628-630, 632-639, 641-647, 651, 657, 660, 662-663, 665-666, 668-671, 673-674, 678, 686-689, 691-693, 695-700, 702-703, 707-709, 711-712, 715, 723, 741, 800-806, 808, 811, 813-821, 823-827, 829-830, 832-833, 835, 844, 846-849, 852, 858-860, 865-866, 868-870, 872-874, 878, 2696-2737 of Table 5.

92. The complex of claim 65, wherein the base editing system comprises an eA3A_T31AT44A of SEQ ID NO: 3206, said guide RNA comprises a spacer corresponding to any one of the protospacers identified as a sequence of Index Nos. 2725-2726 and 2738-2749 of Table 5.

93. The complex of claim 65, wherein the base editing system comprises an evoAPOBEC1-BE4max of SEQ ID NO: 3204, said guide RNA comprises a spacer corresponding to any one of the protospacers identified as a sequence of Index Nos. 1-4, 6-7, 9-11, 13, 15-18, 20, 22-27, 32, 35, 40-42, 44, 47-49, 51-53, 55-56, 58, 61-63, 68, 70-72, 74, 76-82, 84-92, 94-98, 100, 104, 108, 111, 116, 121, 125-126, 131, 136, 141-143, 146-148, 150-151, 153, 155-160, 162, 170, 172, 175, 178-180, 183-184, 190, 195, 198, 200-201, 203-204, 206, 210-212, 214, 217, 220-221, 223-227, 229, 231-233, 235-239, 244, 247, 249, 252-258, 263-270, 272-274, 276, 278-279, 281-284, 286-290, 293-294, 296, 298, 300-301, 304, 318, 321, 324-325, 330-333, 338, 340, 342, 346, 349-351, 358, 363, 373, 379-380, 385-389, 411, 423, 425, 427, 431, 433, 438, 441, 445, 448, 454-455, 459, 463, 465, 472, 476, 480, 482-484, 487, 491, 493-494, 503, 510, 514, 517, 521, 535, 540, 542, 544-545, 551-555, 558-564, 567-568, 573-576, 579-582, 588-589, 593, 595-596, 598, 600, 603, 605, 610, 612-617, 620, 622, 625-626, 628, 630-631, 635-641, 644, 651, 653-654, 656, 676, 678-679, 682, 688, 694, 704, 711, 713-715, 717, 720-723, 728-734, 742-743, 745, 747, 750, 752-754, 756-758, 760, 762, 766, 769-773, 775, 777-779, 781-784, 787, 790-794, 798, 800, 803, 805-806, 809, 811-812, 814, 818-819, 824-825, 827, 829, 831, 833, 835, 838-839, 841-842, 847, 850-855, 857-859, 861, 864, 870-873, 875, 878-879 of Table 5.

94. One or more polynucleotides encoding the complex of any of claims 65-93.

95. A vector comprising the one or more polynucleotides of claim 94 and one or more promoters that drive the expression of the base editor and the guide RNA.

96. A cell comprising the vector of claim 95.

97. A cell comprising a complex of any of claims 65-93.

98. A pharmaceutical composition comprising: (i) a guide RNA selected from the method of claim 1, a complex of any one of claims 65-93, a polynucleotide of claim 94, or a vector of claim 95; and (ii) a pharmaceutically acceptable excipient.

99. A method of editing a target DNA sequence by base editing using a base editor:
selecting a guide RNA for use in the base editing system in accordance with the method of any of claims 1-36; and
contacting the genome of the target DNA sequence with the selected guide RNA and the base editor, thereby editing the target DNA sequence.

100. The method of claim 99, wherein the method is conducted ex vivo, in vivo, or ex vivo.

101. The method of claim 1, wherein the method restores the function of a disease-causing mutation.

102. The method of claim 99, wherein the method of editing introduces a nucleotide change in the target DNA sequence.

103. The method of claim 102, wherein the nucleotide change is a single nucleotide substitution, a deletion, an insertion, or a combination thereof.

104. The method of claim 102, wherein the nucleotide change is a transition mutation.

105. The method of claim 104, wherein the transition mutation is a G to A substitution, a T to C substitution, a C to T substitution, or an A to G substitution.

106. The method of claim 102, wherein the nucleotide change corrects a mutation in a disease-associated gene.

107. The method of claim 106, wherein the disease-associated gene is associated with cardiac disease; high blood pressure; neurological disease; autoimmune disorder, arthritis; diabetes; cancer; or obesity.

108. The method of claim 106, wherein the disease-associated gene is associated with Adenosine Deaminase (ADA) Deficiency; Alpha-1 Antitrypsin Deficiency; Cystic Fibrosis; Duchenne Muscular Dystrophy; Galactosemia; Hemochromatosis; Huntington's Disease; Maple Syrup Urine Disease; Marfan Syndrome; Neurofibromatosis Type 1; Pachyonychia Congenita; Phenylkeotnuria; Severe Combined Immunodeficiency; Sickle Cell Disease; Smith-Lemli-Opitz Syndrome; and Tay-Sachs Disease, or other monogenetic disorder.

109. The library of the training method of claim 37.

110. The library of the training method of claim 38.

111. The method of claim 1, wherein the first machine learning model is trained using training data generated in part using the base editing system.

112. The method of claim 49, further comprising:
prior to performing the applying,
selecting, based on the editing system indicated by the input data, the first machine learning model and the second machine learning model from a plurality of machine learning models.
